(19) United States
(12) Patent Application Publication
(10) Pub. No.: US 2024/0018157 A1
(43) Pub. Date: Jan. 18, 2024

(54) CYCLIC COMPOUNDS AND METHODS OF USING SAME

(71) Applicant: Schrödinger, Inc., New York, NY (US)

(72) Inventors: Shulu Feng, New York, NY (US); Morgan Lawrenz, San Diego, CA (US); Goran Krilov, Long Island, NY (US); Andrew Placzek, Portland, OR (US); Zhe Nie, San Diego, CA (US); Lynnie Trzoss, San Diego, CA (US); Michael Trzoss, San Diego, CA (US); Haifeng Tang, Metuchen, NJ (US); H. Rachel Lagiakos, Brooklyn, NY (US)

(21) Appl. No.: 17/787,835

(22) PCT Filed: Dec. 24, 2020
(86) PCT No.: PCT/US2020/066999

§ 371 Date: Jun. 21, 2022

Related U.S. Application Data
(60) Provisional application No. 63/119,521, filed on Nov. 30, 2020.
(60) Provisional application No. 63/040,582, filed on Jun. 18, 2020.
(60) Provisional application No. 62/954,262, filed on Dec. 27, 2019.

(51) Int. Cl. C07D 487/14; A61P 35/00

(52) U.S. Cl. CPC C07D 487/14; A61P 35/00

(57) ABSTRACT

The present application relates to compounds of Formula (I), as defined herein, and pharmaceutically acceptable salts thereof which are MALT1 inhibitors. The present application also describes pharmaceutical composition comprising a compound of Formula (I), and pharmaceutically acceptable salts thereof, and methods of using the compounds and compositions for treating diseases, such as cancer, autoimmune disorders, and inflammatory disorders.

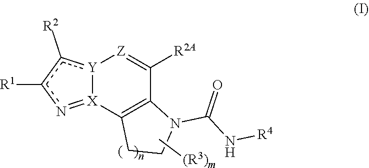

The contents of the text file submitted electronically herewith are incorporated herein by reference in their entirety: A computer readable format copy of the Sequence Listing filename: 17367-0076WO1.txt, date recorded, Dec. 24, 2020, file size≈214 kilobytes.

### TECHNICAL FIELD

This present application relates to tricyclic, and other multi-cyclic compounds that are useful for treating proliferative disorders such as cancer, as well as autoimmune and inflammatory disorders.

### BACKGROUND

MALT1 (mucosa-associated lymphoid tissue lymphoma translocation protein 1) is an intracellular protein involved in lymphocyte proliferation via upstream signaling of NF-κB to control lymphocyte activation, survival, proliferation, and differentiation. Together with a CARMA or CARD scaffold protein, (e.g., CARD11 (caspase recruitment domain family member 11, also known as CARMA1), CARD14 (caspase recruitment domain family member 14, also known as CARMA2), CARD10 (caspase recruitment domain family member 10, also known as CARMA3), or CARD9 (caspase recruitment domain family member 9)) and BCL10 (B-cell CLL/Lymphoma 10), MALT1 is one of the three subunits of the CBM complex which is formed upon cell-surface antigen receptor activation. See Jaworski et al., Cell Mol Life Science 2016, 73, 459-473, and Juilland and Thome. Frontiers in Immunology 2018, 9, 1927. MALT1 is known to mediate NF-κB signaling by at least two mechanisms: firstly, MALT1 functions as a scaffold protein, recruiting NF-κB signaling proteins such as TRAF6, TABs (e.g., TAB1, TAB2, TAB3), TAK1 and NEMO-IKKα/β; and secondly, as a cysteine protease, it cleaves and deactivates negative regulators of NF-κB signaling, such as RelB, A20, or CYLD. See Rosebeck et al., Science, 2011, 331, 468-472.

The protease activity of MALT1 has emerged as a potential therapeutic target, particularly where NF-κB and related pathways are believed to play a significant role. Activated B cell-like diffuse large B cell lymphomas (ABC-DLBCLs) are aggressive lymphomas that are often characterized by NF-κB hyperactivation, and it has been shown that MALT1 protease inhibition can dramatically inhibit growth and promote apoptosis of the highly aggressive ABC type DLBCLs. See Ferch U, et al., J Exp Med 2009, 206, 2313-2320; see also. Hailfinger S, et al., Proc Natl Acad Sci USA 2009, 106, 19946-19951. Known peptide substrates of MALT1, or fusion protein API2-MALT1, include A20, CYLD, BCL10, RelB, regnase-1, roquin-1, NIK, and LIMA 1a. See Rebeaud et al., Nat Immunol 2008, 9, 272-281; see also, Coomaert et al., Nat Immunol 20008, 9, 263-271; Staal et al., EMBO J 2011, 30, 1742-1752; Hailfinger et al., PNAS 2011, 108, 14596-14601; Jeltsch et al., Nat Immunol 2014, 15, 1079-1089; Uehata et al., Cell 2013, 153, 1036-1049; Nie et al., Nat Commun 2015, 6, 5908; and Baens et al., PLoS ONE 2014, 9, e103774. One general profile of MALT1 substrates is described in Kasperkiewicz, et al. Scientific Reports 8.1 (2018): 1-10.

Additionally, several chromosomal translocations that lead to the generation of constitutively active MALT1 have been identified in ABC-DLBCLs and the identification of the MALT1 fusion protein API2-MALT1/IgH-MALT1 that leads to NF-κB activation independent of upstream stimulation further highlights the importance of this protein in cancer and various diseases. See Farinha et al., J Clinical Oncology 2005, 23, 6370-6378. Further, MALT1 has been shown to be involved in several different types of cancers, for example hematological malignancies such as mantle cell lymphoma, chronic lymphocytic leukemia (CLL) and solid tumors such as lung adenocarcinoma, breast cancer, pancreatic cancer, and glioblastoma. See Jiang et al., Cancer Research 2011, 71, 2183-2192; see also. Pan et al., Mol Cancer Res 2016, 14, 93-102, Penas et al., Blood 2010, 115, 2214-2219, and J Cell Mol Med. 2020 July; 24(13):7550-7562. MALT1, as an immunomodulatory protein, is also involved in innate and adaptive immunity and may have effects on several inflammatory disorders, e.g., psoriasis, multiple sclerosis, rheumatoid arthritis, Sjogren's syndrome, ulcerative colitis, and different types of allergic disorders resulting from chronic inflammation. See Afofina et al., FEBS Journal 2015, DOI: 10.1 111/febs. 13325; see also Lowes et al., Ann Review Immunology 2014, 32, 227-255; Jabara et al., J Allergy Clin Immunology 2013, 132, 151-158; Streubel et al., Clin Cancer Research 2004, 10, 476-480; and Liu et al., Oncotarget 2016, 1-14. Recently, findings also suggest the importance of MALT1 in the control of regulatory T cell (Treg) function and homeostasis. Studies are ongoing to confirm the potential of MALT1 inhibitors for the treatment of patients with solid tumors alone or in combination with immune-checkpoint mechanisms. However, no MALT1 inhibitors are currently approved for therapeutic use.

### SUMMARY

Accordingly, provided herein is a compound of the Formula (I):

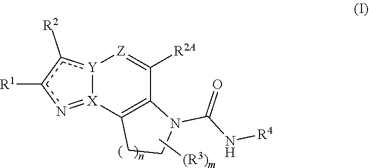

or a pharmaceutically acceptable salt thereof, wherein X, Y, Z, n, R¹, R², R³, m, R⁴, R⁵, R⁶, R^{A}, R^{B}, R^{C}, R^{D}, R^{E}, and R^{F} are as defined herein.

Also provided herein is a pharmaceutical composition comprising a compound of Formula (I), or a pharmaceutically acceptable salt thereof, and at least one pharmaceutically acceptable excipient.

Also provided are methods for treating a CBM complex pathway-associated cancer in a subject in need thereof, comprising administering to the subject an effective amount of a compound of Formula (I), or a pharmaceutically acceptable salt thereof, or a pharmaceutical composition as described herein.

Also provided are methods for treating a cancer in a subject in need thereof, comprising:
(a) identifying the cancer as being a CBM complex pathway-associated cancer; and
(b) administering to the subject an effective amount of a compound of Formula (I), or a pharmaceutically acceptable salt thereof, or a pharmaceutical composition as described herein.

Also provided are methods for treating a cancer in a subject in need thereof, comprising:
administering an effective amount of a compound of Formula (I), or a pharmaceutically acceptable salt thereof, or a pharmaceutical composition as described herein, to a subject identified as having a CBM complex pathway-associated cancer

Also provided are methods for treating a MALT1-associated cancer in a subject, comprising administering to a subject identified or diagnosed as having a MALT1-associated cancer an effective amount of a compound of Formula (I), or a pharmaceutically acceptable salt thereof, or a pharmaceutical composition as described herein.

Also provided are methods for treating cancer in a subject in need thereof, comprising:
(a) determining that the cancer is associated with a dysregulation of a MALT1 gene, a MALT1 protease, or expression or activity or level of any of the same; and
(b) administering to the subject an effective amount of a compound of Formula (I), or a pharmaceutically acceptable salt thereof, or a pharmaceutical composition as described herein.

Also provided are methods for inhibiting metastasis in a subject having a cancer in need of such treatment, comprising administering to the subject an effective amount of a compound of Formula (I), or a pharmaceutically acceptable salt thereof, or a pharmaceutical composition as described herein.

Also provided are methods for treating an autoimmune disorder in a subject in need thereof, comprising administering to the subject an effective amount of a compound of Formula (I), or a pharmaceutically acceptable salt thereof, or a pharmaceutical composition as described herein.

Also provided are methods for treating a CBM complex pathway-associated disease or disorder in a subject in need thereof, comprising administering to the subject an effective amount of a compound of Formula (I), or a pharmaceutically acceptable salt thereof, or a pharmaceutical composition as described herein.

Also provided are methods for treating a disease or disorder in a subject in need thereof, comprising:
(a) identifying the cancer as being a CBM complex pathway-associated disease or disorder; and
(b) administering to the subject an effective amount of a compound of Formula (I), or a pharmaceutically acceptable salt thereof, or a pharmaceutical composition as described herein.

Also provided are methods for treating a disease or disorder in a subject in need thereof, comprising:
administering to the subject an effective amount of a compound of Formula (I), or a pharmaceutically acceptable salt thereof, or a pharmaceutical composition as described herein, to a subject identified as having a CBM complex pathway-associated disease or disorder.

Also provided are methods for treating a MALT1-associated autoimmune disorder in a subject, comprising administering to a subject identified or diagnosed as having a MALT1-associated autoimmune disorder an effective amount of a compound of Formula (I), or a pharmaceutically acceptable salt thereof, or a pharmaceutical composition as described herein.

Also provided are methods for treating a MALT1-associated autoimmune disorder in a subject, comprising administering to a subject identified or diagnosed as having a MALT1-associated autoimmune disorder an effective amount of a compound of Formula (I), or a pharmaceutically acceptable salt thereof, or a pharmaceutical composition as described herein.

Also provided are methods for treating an autoimmune disorder in a subject in need thereof, comprising:
(a) determining that the autoimmune disorder is associated with a dysregulation of a MALT1 gene, a MALT1 protease, or expression or activity or level of any of the same; and
(b) administering to the subject an effective amount of a compound of Formula (I), or a pharmaceutically acceptable salt thereof, or a pharmaceutical composition as described herein.

Also provided are methods for treating a MALT1-associated autoimmune disorder in a subject, comprising administering an effective amount of a compound of Formula (I), or a pharmaceutically acceptable salt thereof, or a pharmaceutical composition as described herein, to a subject determined to have a MALT1-associated autoimmune disorder.

Also provided are methods for treating an inflammatory disorder in a subject in need thereof, comprising administering to the subject an effective amount of a compound of Formula (I), or a pharmaceutically acceptable salt thereof, or a pharmaceutical composition as described herein.

Also provided are methods for treating a MALT1-associated inflammatory disorder in a subject, comprising administering to a subject identified or diagnosed as having a MALT1-associated inflammatory disorder an effective amount of a compound of Formula (I), or a pharmaceutically acceptable salt thereof, or a pharmaceutical composition as described herein.

Also provided are methods for treating a MALT1-associated inflammatory disorder in a subject, comprising administering to a subject identified or diagnosed as having a MALT1-associated inflammatory disorder an effective amount of a compound of Formula (I), or a pharmaceutically acceptable salt thereof, or a pharmaceutical composition as described herein.

Also provided are methods for treating an inflammatory disorder in a subject in need thereof, comprising:
(a) determining that the inflammatory disorder is associated with a dysregulation of a MALT1 gene, a MALT1 protease, or expression or activity or level of any of the same; and
(b) administering to the subject an effective amount of a compound of Formula (I), or a pharmaceutically acceptable salt thereof, or a pharmaceutical composition as described herein.

Also provided are methods for treating a MALT1-associated inflammatory disorder in a subject, comprising administering an effective amount of a compound of Formula (I), or a pharmaceutically acceptable salt thereof, or a pharmaceutical composition as described herein, to a subject determined to have a MALT1-associated inflammatory disorder.

Also provided are methods for inhibiting CBM complex pathway activity in a mammalian cell, comprising contacting the mammalian cell with a compound of Formula (I), or a pharmaceutically acceptable salt thereof.

Also provided are methods for inhibiting MALT1 protease activity in a mammalian cell, comprising contacting the mammalian cell with a compound of Formula (I), or a pharmaceutically acceptable salt thereof.

Also provided are the use of compounds of Formula (I), or pharmaceutically acceptable salts thereof, for treating a CBM complex pathway-associated disease or disorder.

Also provided are compounds of Formula (I), or pharmaceutically acceptable salts thereof, for use in the manufacture of a medicament for the treatment of a CBM complex pathway-associated disease or disorder.

Also provided are methods of treating an individual with a MALT1-associated cancer that include administering a compound of Formula (I), or a pharmaceutically acceptable salt thereof, before, during, or after administration of other anticancer drug(s) (e.g., a first MALT1 inhibitor or another MALT1 inhibitor).

Also provided herein is a process for preparing a compound of Formula (I), or a pharmaceutically acceptable salt thereof.

Also provided herein is a compound of Formula (I), or a pharmaceutically acceptable salt thereof obtained by a process of preparing the compound as defined herein.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entireties. In case of conflict, the present specification, including definitions, will control.

Other features and advantages of the disclosure will be apparent from the following detailed description and from the claims.

### DETAILED DESCRIPTION

### Definitions

The term “compound,” as used herein is meant to include all stereoisomers, geometric isomers, tautomers, and isotopically enriched variants of the structures depicted. Compounds herein identified by name or structure as one particular tautomeric form are intended to include other tautomeric forms unless otherwise specified.

The term “tautomer,” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer. The following is an example of included tautomeric forms:

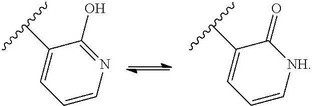 [SMILES: *c1cccnc1O ; *c1ccc[nH]c1=O]

It will be appreciated that certain compounds provided herein may contain one or more centers of asymmetry and may therefore be prepared and isolated in a mixture of isomers such as a racemic mixture, or in an enantiomerically pure form.

The term “halo” refers to one of the halogens, group 17 of the periodic table. In particular, the term refers to fluorine, chlorine, bromine and iodine. Preferably, the term refers to fluorine or chlorine.

The term “C1-C6 alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl and n-hexyl. Similarly, a C1-C3 alkyl group is linear or branched hydrocarbon chain containing 1, 2, or 3 carbon atoms.

The term “C1-C6 haloalkyl” refers to a hydrocarbon chain substituted with at least one halogen atom independently chosen at each occurrence, for example fluorine, chlorine, bromine and iodine. The halogen atom may be present at any position on the hydrocarbon chain. Similarly, a C1-C3 haloalkyl group is linear or branched hydrocarbon chain containing 1, 2, or 3 carbon atoms substituted with at least one halogen atom. For example, C1-C3 haloalkyl may refer to chloromethyl, fluoromethyl, trifluoromethyl, chloroethyl e.g. 1-chloroethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl.

The term “C1-C6 alkoxy” refers to a C1-C6 alkyl group which is attached to a molecule via oxygen. This includes moieties where the alkyl part may be linear or branched, such as methoxy, ethoxy, n-propoxy, iso-propoxy, n-butoxy, sec-butoxy, tert-butoxy, n-pentoxy and n-hexoxy.

The term “C1-C6 haloalkoxy” refers to a C1-C6 alkyl group which is attached to a molecule via oxygen and where at least one hydrogen atom of the alkyl group is replaced with a halogen. This includes moieties where the alkyl part may be linear or branched, such as fluoromethoxy, difluoromethoxy, trifluoromethoxy, 2,2,2-trifluoroethoxy, or trifluoropropoxy.

A [symbol] represents a single or double bond, valence permitting. For example,

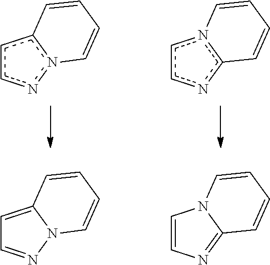

As used herein, the term “cyano” refers to a —CN radical.

As used herein, the term “hydroxyl” refers to an —OH radical.

As used herein, the term “amino” refers to an —NH₂ group.

As used herein, the term “aryl” refers to a 6-10 all carbon mono- or bicyclic group wherein at least one ring in the system is aromatic. Non-limiting examples of aryl groups include phenyl, naphthyl, tetrahydronaphthyl. In bicyclic ring systems where only one ring is aromatic, the non-aromatic ring can be a cycloalkyl group, as defined herein.

As used herein, the term “heteroaryl” refers to a 5-10 membered mono- or bicyclic group wherein at least one ring in the system is aromatic; wherein one or more carbon atoms in at least one ring in the system is/are replaced with an heteroatom independently selected from N, O, and S. Heteroaryl groups include rings where one or more groups are oxidized, such as a pyridone moiety. Non-limiting examples of heteroaryl groups include pyridine, pyrimidine, pyrrole, imidazole, and indole. In bicyclic ring systems where only one ring is aromatic, the non-aromatic ring can be a cycloalkyl or heterocyclyl group, as defined herein.

As used herein, the term “cycloalkyl” refers to a saturated or partially unsaturated 3-10 mono- or bicyclic hydrocarbon group; wherein bicyclic systems include fused, spiro (optionally referred to as “spirocycloalkyl” groups), and bridged ring systems. Non-limiting examples of cycloalkyl groups include cyclopropyl, cyclohexyl, spiro[2.3]hexyl, and bicyclo[1.1.1]pentyl.

The term “heterocyclyl” refers to a saturated or partially unsaturated 3-12 membered hydrocarbon monocyclic or bicyclic ring system, that is not aromatic, having at least one heteroatom within the ring selected from N, O and S. Bicyclic heterocyclyl groups include fused, spiro (optionally referred to as “spiroheterocyclyl” groups), and bridged ring systems. The heterocyclyl ring system may include oxo substitution at one or more C, N, or S ring members. The heterocyclyl group may be denoted as, for example, a “5-10 membered heterocyclyl group,” which is a ring system containing 5, 6, 7, 8, 9 or 10 atoms at least one being a heteroatom. For example, there may be 1, 2 or 3 heteroatoms, optionally 1 or 2. The heterocyclyl group may be bonded to the rest of the molecule through any carbon atom or through a heteroatom such as nitrogen. Exemplary heterocyclyl groups include, but are not limited to, piperidinyl, piperazinyl, morpholino, tetrahydropyranyl, azetidinyl, oxetanyl, 2-azaspiro[3.3]heptanyl, pyrrolidin-2-one, sulfolane, isothiazoline S,S-dioxide, and decahydronaphthalenyl.

As used herein, the term “geminal” refers to substituent atoms or groups attached to the same atom in a molecule.

As used herein, the term “vicinal” refers to substituent atoms or groups attached to adjacent atoms in a molecule. The stereochemical relationship between the substituent atoms or groups can be cis, trans, undefined, or unresolved.

As used herein, the term “oxo” refers to an “═O” group attached to a carbon atom.

As used herein, the symbol [symbol] depicts the point of attachment of an atom or moiety to the indicated atom or group in the remainder of the molecule.

It is to be understood that the ring in compounds of Formula (I) comprising atoms X, Y and Z does not contain more than two adjacent nitrogen atoms.

The compounds of Formula (I) include pharmaceutically acceptable salts thereof. In addition, the compounds of Formula (I) also include other salts of such compounds which are not necessarily pharmaceutically acceptable salts, and which may be useful as intermediates for preparing and/or purifying compounds of Formula (I) and/or for separating enantiomers of compounds of Formula (I). Non-limiting examples of pharmaceutically acceptable salts of compounds of Formula (I) include trifluoroacetic acid and hydrochloride salts.

It will further be appreciated that the compounds of Formula (I) or their salts may be isolated in the form of solvates, and accordingly that any such solvate is included within the scope of the present disclosure. For example, compounds of Formula (I) and salts thereof can exist in unsolvated as well as solvated forms with pharmaceutically acceptable solvents such as water, ethanol, and the like.

In some embodiments, the compounds of Formula (I) include the compounds of Examples 1-211 and stereoisomers and pharmaceutically acceptable salts thereof. In some embodiments, the compounds of Formula (I) include the compounds of Examples 1-211 and pharmaceutically acceptable salts thereof. In some embodiments, the compounds of Examples 1-211 are in the free base form. In some embodiments, the compounds of Examples 1-211 are in the form of pharmaceutically acceptable salts.

The term “pharmaceutically acceptable” indicates that the compound, or salt or composition thereof is compatible chemically and/or toxicologically with the other ingredients comprising a formulation and/or the subject being treated therewith.

Protecting groups can be a temporary substituent which protects a potentially reactive functional group from undesired chemical transformations. The choice of the particular protecting group employed is well within the skill of one of ordinary skill in the art. A number of considerations can determine the choice of protecting group including, but not limited to, the functional group being protected, other functionality present in the molecule, reaction conditions at each step of the synthetic sequence, other protecting groups present in the molecule, functional group tolerance to conditions required to remove the protecting group, and reaction conditions for the thermal decomposition of the compounds provided herein. The field of protecting group chemistry has been reviewed (Greene, T. W. and Wuts, P. G. M. Protective Groups in Organic Synthesis, 2.sup. ed. Wiley: New York, 1991).

A nitrogen protecting group can be any temporary substituent which protects an amine moiety from undesired chemical transformations. Examples of moieties formed when such protecting groups are bonded to an amine include, but are not limited to allylamine, benzylamines (e.g., bezylamine, p-methoxybenzylamine, 2,4-dimethoxybenzylamine, and tritylamine), acetylamide, trichloroacetammide, trifluoroacetamide, pent-4-enamide, phthalimides, carbamates (e.g., methyl carbamate, t-butyl carbamate, benzyl carbamate, allyl carbamates, 2,2,2-trichloroethyl carbamate, and 9-fluorenylmethyl carbamate), imines, and sulfonamides (e.g., benzene sulfonamide, p-toluenesulfonamide, and p-nitrobenzenesulfonamide).

An oxygen protecting group can be any temporary substituent which protects a hydroxyl moiety from undesired chemical transformations. Examples of moieties formed when such protecting groups are bonded to a hydroxyl include, but are not limited to esters (e.g., acetyl, t-butyl carbonyl, and benzoyl), benzyl (e.g., benzyl, p-methoxybenzyl, and 2,4-dimethoxybenzyl, and trityl), carbonates (e.g., methyl carbonate, allyl carbonate, 2,2,2-trichloroethyl carbonate and benzyl carbonate) ketals, and acetals, and ethers.

Compounds provided herein may also contain unnatural proportions of atomic isotopes at one or more of the atoms that constitute such compounds. That is, an atom, in particular when mentioned in relation to a compound according to Formula (I), comprises all isotopes and isotopic mixtures of that atom, either naturally occurring or synthetically produced, either with natural abundance or in an isotopically enriched form. For example, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof; when nitrogen is mentioned, it is understood to refer to ¹³N, ¹⁴N, ¹⁵N or mixtures thereof; when oxygen is mentioned, it is understood to refer to ¹⁴O, ¹⁵O, ¹⁶O, ¹⁷O, ¹⁸O or mixtures thereof; and when fluoro is mentioned, it is understood to refer to ¹⁸F, ¹⁹F or mixtures thereof; unless expressly noted otherwise. For example, in deuteroalkyl and deuteroalkoxy groups, where one or more hydrogen atoms are specifically replaced with deuterium (²H). As some of the aforementioned isotopes are radioactive, the compounds provided herein therefore also comprise compounds with one or more isotopes of one or more atoms, and mixtures thereof, including radioactive compounds, wherein one or more non-radioactive atoms has been replaced by one of its radioactive enriched isotopes. Radiolabeled compounds are useful as therapeutic agents, e.g., cancer therapeutic agents, research reagents, e.g., assay reagents, and diagnostic agents, e.g., in vivo imaging agents. All isotopic variations of the compounds provided herein, whether radioactive or not, are intended to be encompassed within the scope of the present disclosure.

For illustrative purposes, general methods for preparing the compounds are provided herein as well as key intermediates. For a more detailed description of the individual reaction steps, see the Examples section below. Those skilled in the art will appreciate that other synthetic routes may be used to synthesize the inventive compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

The ability of selected compounds to act as MALT1 inhibitors may be demonstrated by the biological assays described herein. IC₅₀ values are shown in Table A.

Compounds of Formula (I), or a pharmaceutically acceptable salt thereof, are useful for treating diseases and disorders which can be treated with a MALT1 inhibitor, such as MALT1-associated cancers, including hematological cancers and solid tumors, MALT1-associated autoimmune disorders, and MALT1-associated inflammatory disorders.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the term “subject” refers to any animal, including mammals such as humans. In some embodiments, the subject is a human. In some embodiments, the subject has experienced and/or exhibited at least one symptom of the disease or disorder to be treated and/or prevented.

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

In certain embodiments, compounds of Formula (I), or a pharmaceutically acceptable salt thereof are useful for preventing diseases and disorders as defined herein (for example, autoimmune disorders, inflammatory disorders, and cancer). The term “preventing” as used herein means the prevention of the onset, recurrence or spread, in whole or in part, of the disease or condition as described herein, or a symptom thereof.

The term “regulatory agency” refers to a country's agency for the approval of the medical use of pharmaceutical agents with the country. For example, a non-limiting example of a regulatory agency is the U.S. Food and Drug Administration (FDA).

Signaling through the NF-κB pathway has been implicated in many cancers. See. e.g., Staudt, Cold Spring Harbor Perspectives in Biology 2.6 (2010): a000109, Xia, et al. Cancer Immunol. Res. 2.9 (2014): 823-830, Xia, et al. OncoTargets and Therapy 11 (2018): 2063. NF-κB is a family of transcription factors, including p50, p52, p65 (ReIA), RelB, and c-Rel, which can bindto the kB enhancer element as various homo- and heterodimers to induce transcription of a number of genes. Following activation of certain cell-surface receptors (e.g., CD28, BCR, HER1 (also known as EGFR (Epidermal Growth Factor Receptor) and ERBB1), or HER2 (also known as HER2/neu or ERBB2)), a CBM complex is formed via phosphorylation of a CARD or CARMA protein, likely by a protein kinase C (e.g., protein kinase C beta or protein kinase C theta) and recruitment of the BCL10-MALT1 complex. See. e.g., Xia, et al. OncoTargets and Therapy 11 (2018): 2063, Shi, and Sun. Mol. Immunol. 68.2 (2015): 546-557, Xia, et al. Cancer Immunol. Res. 2.9 (2014): 823-830, and Pan, Mol. Cancer Res. 14.1 (2016): 93-102.

As noted hereinabove, the CBM complex can function as a scaffold protein in the activation of the NF-κB pathway. When formed, the CBM complex can activate the IKK complex (e.g., IKKγ (also called NEMO), IKKα, and IKKβ), likely by ubiquintination (e.g., K63-linked ubiquitination) of MALT1, which results in the recruitment, ubiquitination (e.g., K63-linked ubiquitination), and degredation of IKKγ, thereby releasing IKKα and IKKβ to phosphorylate IκB, resulting in the ubiquitination (e.g., K48-linked ubiquitination) and degradation of IκB, releasing the NF-κB transcription factors (typically of the NF-κB1 subtype: p50-RelA and p50-cRel) to the nucleus. This cascade is likely mediated by the ubiquitin ligase TRAF6 (Tumor necrosis factor receptor (TNFR)-associated factor 6). The CBM complex may also affect NF-κB signaling through addtitional protein complexes, such as TAB1/2-TAK and the linear ubiquitin chain assembly complex (LUBAC). See. e.g., Israel, Cold Spring Harbor Perspectives in Biology 2.3 (2010): a000158, Xia, et al. OncoTargets and Therapy 11 (2018): 2063, Juilland, Front. Immunol. 9 (2018): 1927. MALT1 can also activate the JNK pathway (also called the JNK/AP-1 pathway), though less work has been done to study this area. See, e.g., Juilland, Front. Immunol. 9 (2018): 1927, and Wang, et al., Oncogenesis 6.7 (2017): e365-e365.

In addition, MALT1 has cysteine protease activity. Non-limiting examples of substrates of wild-type MALT1 include BCL10, A20, CYLD, RelB, Regnase 1, roquin-1, and HOIL1. In addition, the API2-MALT1 (also called cIAP2; amino terminus of inhibitor of apoptosis 2) fusion protein has also been shown to cleave NIK and LIMA1α. BCL10 cleavage by MALT1 is believed to result in BCL10-independent NF-κB activation. By cleaving A20 (TNF Alpha Induced Protein 3), MALT1 can reduce negative regulation of the NF-κB pathway, as A20 is a deubiquitinating enzyme that has been suggested to reduce the ubiquitination of MALT1 and thus recruitment and activation of the IKK complex. CYLD (CYLD Lysine 63 Deubiquitinase) is a deubiquitinating enzyme, and by cleavage of this enzyme, it is believed that MALT1 increases signaling through the NF-κB pathway and/or JNK pathway. Cleavage of RelB typically results in relief of negative regulation of the NF-κB pathway, as RelB forms transcriptionally inactive complexes with RelA and c-Rel. By cleaving HOIL1 (also known as RBCK1), it is believed that negative regulation of the NF-κB is relieved, as HOIL1 is thought to decrease linear ubiquitination. MALT1 can also autoprocess, which promotes signaling through the NF-κB pathway through a mechanism that is not fully understood. By cleaving NIK (NF-κB inducing kinase), the API2-MALT1 protease generates a c-terminal fragment of NIK that is resistant to proteasomal degradation and thereby increases noncanonical NF-κB signaling. By cleaving LIMA1α (LIM domain and actin-binding protein 1), the tumor-suppressing properties of this protein are diminished, and it believed that the remaining fragment has oncogenic properties and enhances cell proliferation, colony formation, and cell adhesion. Cleavage of Regnase 1 (Regulatory RNase 1, also known as MCPIP-1 or Zc3h12a), and roquin-1 (also known as RC3H1) is believed to result in the stabilization of mRNAs, including those of cytokines, chemokines, and costimulatory proteins such as ICOS, OX40, and TNF. This activity may be independent of MALT1 activity in the NF-κB and JNK pathways. See, e.g., Afonina, et al. FEBS J. 282.17 (2015): 3286-3297 Klein et al. Nat. Comm. 6.1 (2015): 1-17, Baens, et al. PloS one 9.8 (2014): e103774, and Juilland, Front. Immunol. 9 (2018): 1927. MALT1 is also involved in oncogenic BCR signalling in ibrutinib-responsive cell lines and biopsie samples, coordinated by a multiprotein supercomplex formed by MYD88, TLR9 and the BCR (hereafter termed the My-T-BCR supercomplex). The My-T-BCR supercomplex co-localizes with mTOR on endolysosomes, where it drives pro-survival NF-κB and mTOR signalling. See Phelan et al., Nature 2018 August; 560(7718):387-391.

Accordingly, inhibition of MALT1 can provide beneficial effects to many types of disorders associated with aberrant signaling in the NF-κB pathway or JNK pathway. For example, inhibition of MALT1 can decrease flux through the NF-κB or JNK pathways resulting from one or more of:
(1) An inactivated tumor suppressor gene. Non-limiting examples of tumor suppressor genes that can be inactivated include BRCA1 and p53 (e.g., p53 H61L or I123T). See. e.g., Sau, et al. Cell Stem Cell 19.1 (2016): 52-65, Xia, et al. Cancer Immunol. Res. 2.9 (2014): 823-830, Johansson, et al. Oncotarget 7.38 (2016): 62627.
(2) A dysregulated cell surface receptor. Non-limiting examples of cell surface receptors include HER1 and HER2. See, e.g., Xia, et al. Cancer Immunol. Res. 2.9 (2014): 823-830 and Pan, Mol. Cancer Res. 14.1 (2016): 93-102.
(3) Dysreguation of one or more components of a CBM complex. Non-limiting examples of components ofa CBM complex include MALT1, CARD11, CARD14, CARD10, CARD9, and BCL10.
(4) Dysregulation of one or more substrates of a MALT1 protease (e.g., a wild-type MALT1 protease or a dysregulated MALT1 protease). Non-limiting examples of substrates of a MALT1 protease include BCL10, A20, CYLD, RelB, Regnase 1, roquin-1, HOIL1, NIK, and LIMA1a.
(5) Dysregulation of one or more components of the NF-κB pathway downstream of a CBM complex. Non-limiting examples of a component of the NF-κB pathway downstream of a CBM complex include TRAF6, IKKα, IKKβ, IKKγ (also called NEMO), IkBα, p50, p52, p65 (RelA), RelB, and c-Rel.
(6) Dysregulation of one or more components of the JNK pathway downstream of a CBM complex. Non-limiting examples of a component of the JNK pathway downstream of a CBM complex include JNK1 (Mitogen-Activated Protein Kinase 8), JNK2 (Mitogen-Activated Protein Kinase 9), JNK3 (Mitogen-Activated Protein Kinase 10), or an AP-1 transcription factor (e.g., a heterodimer of any of the c-Fos, c-Jun, ATF, or JDP families).
(7) Dysregulation of one or more fusion proteins caused by chromosome translocation of MALT1 gene. Non-limiting example includes the cIAP-MALT1 fusion protein.
(8) Dysregulation of one or more components of the My-T-BCR supercomplex. Non-limiting examples of a component of the My-T-BCR supercomplex include MYD88, TLR9, and mTOR.

The term “CBM complex pathway” as associated herein includes genes, transcripts, and proteins in a signaling pathway that includes a CBM. For example, many aspects of the NF-κB pathway are part of a CBM complex pathway. A CBM complex pathway can include, for example, cell surface receptors (e.g., CD28, BCR, HER1, and HER2), a signal transducer between a cell surface receptor and a CBM complex (e.g, a protein kinase C beta or protein kinase C theta), a component of a CBM complex (e.g., MALT1, CARD1l, CARD14, CARD10, CARD9, or BCL10), substrates of a MALT1 protease (e.g., BCL10, A20, CYLD, RelB, Regnase 1, roquin-1, HOIL1, NIK, and LIMA1α), a component of the NF-κB pathway downstream of a CBM complex (e.g., TAK1, TRAF6, TAB1, TAB2, TAB3, MKK7, IKKα, IKKβ, IKKγ, IkBα, p50, p65 (RelA), or c-Rel), a component of the JNK pathway downstream of a CBM complex (e.g., JNK1, JNK2, JNK3, or an AP-1 transcription factor), or a components of the My-T-BCR supercomplex (e.g., MYD88, TLR9, or mTOR).

As used herein, the term “CBM complex pathway-associated disease or disorder” refers to diseases or disorders associated with or having a dysregulation of a gene in a CBM complex pathway, a protein in a CBM complex pathway, or the expression or activity or level of any (e.g., one or more) of the same (e.g., any of the types of dysregulation of a gene in a CBM complex pathway, a protein in a CBM complex pathway, or the expression or activity or level of any of the same, as described herein). Non-limiting examples of a CBM complex pathway-associated diseases or disorders include, for example, CBM-related primary immunodeficiency diseases, autoimmune disorders, multiple sclerosis, colitis, psoriasis, and cancer. See. e.g., McGuire, et al. J. Neuroinflamm. 11.1 (2014): 1-12, Lu, et al., Front. Immunol. 9 (2018): 2078, Jaworski, et al., EMBO J. 33.23 (2014): 2765-2781. Non-limiting examples of a CBM complex pathway-associated disease or disorder include MALT1-associated diseases or disorders such as MALT1-associated cancers, MALT1-associated autoimmune disorders, and MALT1-associated inflammatory disorders.

The term “CBM complex pathway-associated autoimmune disorder” as used herein refers to autoimmune disorders associated with or having a dysregulation of a CBM complex pathway gene, a CBM complex pathway protein, or the expression or activity or level of any (e.g., one or more) of the same (e.g., any of the types of dysregulation of a CBM complex pathway gene, a CBM complex pathway protein, or the expression or activity or level of any of the same described herein). Non-limiting examples of a CBM complex pathway-associated autoimmune disorders are described herein.

The term “CBM complex pathway-associated inflammatory disorder” as used herein refers to inflammatory disorders associated with or having a dysregulation of a CBM complex pathway gene, a CBM complex pathway protein, or the expression or activity or level of any (e.g., one or more) of the same (e.g., any of the types of dysregulation of a CBM complex pathway gene, a CBM complex pathway protein, or the expression or activity or level of any of the same described herein). Non-limiting examples of a CBM complex pathway-associated inflammatory disorders are described herein.

In some embodiments, a CBM complex pathway-associated disease or disorder is a CBM complex pathway-associated cancer, such as a CBM complex pathway cell surface receptor-associated cancer (e.g., a CD28-associated cancer, a BCR-associated cancer, a HER1-associated cancer, or a HER2-associated cancer), a cancer associated with a signal transducer between a cell surface receptor and a CBM complex (e.g, a protein kinase C beta (PKCβ)-associated cancer or a protein kinase C theta (PCKθ)-associated cancer), a component of a CBM complex-associated cancer (e.g., a MALT1-associated cancer, a CARD11-associated cancer, a CARD14-associated cancer, a CARD10-associated cancer, a CARD9-associated cancer, or a BCL10-associated cancer), a MALT1 protease substrate-associated cancer (e.g., a BCL10-associated cancer, an A20-associated cancer, a CYLD-associated cancer, a RelB-associated cancer, a Regnase 1-associated cancer, a roquin-1-associated cancer, a HOIL1-associated cancer, a NIK associated cancer, or a LIMA1α-associated cancer), a cancer associated with a component of the NF-κB pathway downstream of a CBM complex (e.g., TAK1-associated cancer, a TRAF6-associated cancer, a TAB1-associated cancer, a TAB2-associated cancer, a TAB3-associated cancer, a MKK7-associated cancer, an IKKα-associated cancer, an IKKβ-associated cancer, an IKKγ-associated cancer, an IkBα-associated cancer, a p50-associated cancer, a p65 (RelA)-associated cancer, or a c-Rel-associated cancer), a cancer associated with a component of the JNK pathway downstream of a CBM complex (e.g., a JNK1-associated cancer, a JNK2-associated cancer, a JNK3-associated cancer, or an AP-1 transcription factor-associated cancer), a MYD88-associated cancer, or a combination thereof.

The term “CBM complex pathway-associated cancer” as used herein refers to cancers associated with or having a dysregulation of a gene in a CBM complex pathway, a protein in a CBM complex pathway, or the expression or activity or level of any (e.g., one or more) of the same (e.g., any of the types of dysregulation of a gene in a CBM complex pathway, a protein in a CBM complex pathway, or the expression or activity or level of any of the same, as described herein) (e.g., upon diagnosis or after developing resistance to previous therapies). Non-limiting examples of a CBM complex pathway-associated cancer are described herein. In some embodiments, a CBM pathway-associated cancer can be a CBM complex pathway cell surface receptor-associated cancer (e.g., a CD28-associated cancer, a BCR-associated cancer, a HER1-associated cancer, or a HER2-associated cancer), a cancer associated with a signal transducer between a cell surface receptor and a CBM complex (e.g, a protein kinase C beta (PKCβ)-associated cancer or a protein kinase C theta (PCKθ)-associated cancer, a component of a CBM complex-associated cancer (e.g., a MALT1-associated cancer, a CARD11-associated cancer, a CARD14-associated cancer, a CARD10-associated cancer, a CARD9-associated cancer, or a BCL10-associated cancer), a MALT1 protease substrate-associated cancer (e.g., a BCL10-associated cancer, an A20-associated cancer, a CYLD-associated cancer, a RelB-associated cancer, a Regnase 1-associated cancer, a roquin-1-associated cancer, a HOIL1-associated cancer, a NIK associated cancer, or a LIMA1α-associated cancer), a cancer associated with a component of the NF-κB pathway downstream of a CBM complex (e.g., TAK1-associated cancer, a TRAF6-associated cancer, a TAB1-associated cancer, a TAB2-associated cancer, a TAB3-associated cancer, a MKK7-associated cancer, an IKKα-associated cancer, an IKKβ-associated cancer, an IKKγ-associated cancer, an IkBα-associated cancer, a p50-associated cancer, a p65 (RelA)-associated cancer, or a c-Rel-associated cancer), a cancer associated with a component of the JNK pathway downstream of a CBM complex (e.g., a JNK1-associated cancer, a JNK2-associated cancer, a JNK3-associated cancer, or an AP-1 transcription factor-associated cancer), or a combination thereof.

In some embodiments, a dysregulation can be a dysregulation that results in aberrant activation of a gene, protein, or expression or activity or level of any of the same. Activation can be through any appropriate mechanism, including, but not limited to, gene amplification, activating mutation, activating translocation, transcriptional activation, epigenetic alteration, and/or overexpression of the protein product of the oncogene. In some embodiments, a dysregulation can be a dysregulation that results in aberrant inactivation of a gene, protein, or expression or activity or level of any of the same. Inactivation can be through any appropriate mechanism, including, but not limited to, gene deletion, inactivating mutation, inactivating translocation, transcriptional silencing, epigenetic alteration, and degradation of mRNA and/or protein products of the gene. Typically, as used herein, a dysregulation, whether it be activation or inactivation, is a dysregulation that results in increased signaling through the NF-κB or JNK signaling pathways.

The term “wild-type” describes a nucleic acid (e.g., a MALT1 gene or a MALT1 mRNA) or protein (e.g., a MALT1 protein) that is found in a subject that does not have a disease or disorder associated with the nucleic acid or the protein (e.g., the MALT1 gene, MALT1 mRNA, or MALT1 protein) (and optionally also does not have an increased risk of developing a disease or disorder associated with the nucleic acid or the protein and/or is not suspected of having a disease or disorder associated with the gene or the protein), or is found in a cell or tissue from a subject that does not have a disease or disorder associated with the gene or the protein (e.g., a MALT1-associated cancer, autoimmune disorder, or inflammatory disorder) (and optionally also does not have an increased risk of developing a disease or disorder associated with the nucleic acid or the protein and/or is not suspected of having a disease or disorder associated with the nucleic acid or the protein.

In some embodiments, the subject has been identified or diagnosed as having a cancer with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated cancer)(e.g., as determined using a regulatory agency-approved, e.g., FDA-approved, assay or kit). In some embodiments, the subject has has a cancer resistant to one or more previous therapies. In some embodiments, the subject has a tumor that is positive for a dysregulation of a CBM complex pathway-associated gene (e.g., a MALT1 gene), a CBM complex pathway-associated protein (e.g., a MALT1 protein), or expression or activity, or level of any of the same (e.g., as determined using a regulatory agency-approved, e.g., FDA-approved, assay or kit). The subject can be a subject with a tumor(s) that is positive for a dysregulation of a CBM complex pathway-associated gene (e.g., a MALT1 gene), a CBM complex pathway-associated protein (e.g., a MALT1 protein), or expression or activity, or level of any of the same (e.g., identified as positive using a regulatory agency-approved, e.g., FDA-approved, assay or kit). The subject can be a subject whose tumors have a dysregulation of a CBM complex pathway-associated gene (e.g., a MALT1 gene), a CBM complex pathway-associated protein (e.g., a MALT1 protein), or expression or activity, or a level of the same (e.g., where the tumor is identified as such using a regulatory agency-approved, e.g., FDA-approved, kit or assay). In some embodiments, the subject has a tumor resistant to one or more previous therapies. In some embodiments, the subject is suspected of having a CBM complex pathway-associated-associated cancer. In some embodiments, the subject has a tumor that is suspected of being resistant to one or more previous therapies. In some embodiments, the subject has a clinical record indicating that the subject has a tumor that has a dysregulation of a CBM complex pathway-associated gene (e.g., a MALT1 gene), a CBM complex pathway-associated protein (e.g., a MALT1 protein), or expression or activity, or level of any of the same (and optionally the clinical record indicates that the subject should be treated with any of the compositions provided herein). In some embodiments, the subject is a pediatric subject. In some embodiments, the subject has a clinical record indicating that the subject has a tumor resistant to one or more previous therapies. In some embodiments, the subject has been identified or diagnosed as having a cancer that, based on histological examination, is determined to be associated with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated cancer).

In some embodiments, the subject has been identified or diagnosed as having an autoimmune disorder with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated autoimmune disorder) (e.g., as determined using a regulatory agency-approved, e.g., FDA-approved, assay or kit). In some embodiments, the subject has a tumor that is positive for a dysregulation of a CBM complex pathway-associated gene (e.g., a MALT1 gene), a CBM complex pathway-associated protein (e.g., a MALT1 protein), or expression or activity, or level of any of the same (e.g., as determined using a regulatory agency-approved, e.g., FDA-approved, assay or kit). In some embodiments, the subject is suspected of having a CBM complex pathway-associated-associated autoimmune disorder. In some embodiments, the subject has a clinical record indicating that the subject has a tumor that has a dysregulation of a CBM complex pathway-associated gene (e.g., a MALT1 gene), a CBM complex pathway-associated protein (e.g., a MALT1 protein), or expression or activity, or level of any of the same (and optionally the clinical record indicates that the subject should be treated with any of the compositions provided herein). In some embodiments, the subject is a pediatric subject. In some embodiments, the subject has been identified or diagnosed as having an autoimmune disorder that, based on histological examination, is determined to be associated with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated autoimmune disorder).

In some embodiments, the subject has been identified or diagnosed as having an inflammatory disorder with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated inflammatory disorder) (e.g., as determined using a regulatory agency-approved, e.g., FDA-approved, assay or kit). In some embodiments, the subject has a tumor that is positive for a dysregulation of a CBM complex pathway-associated gene (e.g., a MALT1 gene), a CBM complex pathway-associated protein (e.g., a MALT1 protein), or expression or activity, or level of any of the same (e.g., as determined using a regulatory agency-approved, e.g., FDA-approved, assay or kit). In some embodiments, the subject is suspected of having a CBM complex pathway-associated-associated inflammatory disorder. In some embodiments, the subject has a clinical record indicating that the subject has a tumor that has a dysregulation of a CBM complex pathway-associated gene (e.g., a MALT1 gene), a CBM complex pathway-associated protein (e.g., a MALT1 protein), or expression or activity, or level of any of the same (and optionally the clinical record indicates that the subject should be treated with any of the compositions provided herein). In some embodiments, the subject is a pediatric subject. In some embodiments, the subject has been identified or diagnosed as having an inflammatory disorder that, based on histological examination, is determined to be associated with a dysregulation of a CBM complex pathway-associated gene (e.g., a MALT1 gene), a CBM complex pathway-associated protein (e.g., a MALT1 protein), or expression or activity, or level of any of the same (a CBM complex pathway-associated-associated inflammatory disorder).

The term “CBM complex pathway cell surface receptor-associated cancer” as used herein refers to cancers associated with or having a dysregulation of a gene, a protein, or the expression or activity or level of any (e.g., one or more) of the same associated with a CBM complex pathway cell surface receptor. In some embodiments, a CBM complex pathway cell surface receptor-associated cancer is selected from the group consisting of a CD28-associated cancer, a BCR-associated cancer, a HER1-associated cancer, a HER2-associated cancer, and combinations thereof.

The term “*-associated cancer” as used herein refers to cancers associated with or having a dysregulation of a * gene, a * protein, or the expression or activity or level of any (e.g., one or more) of the same (e.g., any of the types of dysregulation of a * gene, a * protein, or the expression or activity or level of any of the same described herein), where “*” refers to a particular CBM complex pathway gene or protein, described herein. In some embodiments, the *-associated cancer is selected from the group consisting of: CD28-associated cancer, BCR-associated cancer, HER1-associated cancer, HER2-associated cancer, PKCβ-associated cancer, PKCθ-associated cancer, MALT1-associated cancer, CARD11-associated cancer, CARD14-associated cancer, A20-associated cancer, CYLD-associated cancer, RelB-associated cancer, HOIL1-associated cancer, NIK-associated cancer, Regnase 1-associated cancer, LIMA1α-associated cancer, roquin-1-associated cancer, TRAF6-associated cancer, TAK1-associated cancer, TAB1-associated cancer, TAB2-associated cancer, TAB3-associated cancer, MKK7-associated cancer, IKKα-associated cancer, IKKβ-associated cancer, IKKγ-associated cancer, IkBα-associated cancer, p50-associated cancer, p65-associated cancer, c-Rel-associated cancer, JNK1-associated cancer, JNK2-associated cancer, JNK3-associated cancer, MYD88 transcription factor-associated cancer, and an AP-1 transcription factor-associated cancer. In some embodiments, the *-associated cancer is a CD28-associated cancer. In some embodiments, the *-associated cancer is a BCR-associated cancer. In some embodiments, the *-associated cancer is a HER1-associated cancer. In some embodiments, the *-associated cancer is a HER2-associated cancer. In some embodiments, the *-associated cancer is a PKCβ-associated cancer. In some embodiments, the *-associated cancer is a PKCθ-associated cancer. In some embodiments, the *-associated cancer is a MALT1-associated cancer. In some embodiments, the *-associated cancer is a CARD11-associated cancer. In some embodiments, the *-associated cancer is a CARD14-associated cancer. In some embodiments, the *-associated cancer is an A20-associated cancer. In some embodiments, the *-associated cancer is a CYLD-associated cancer. In some embodiments, the *-associated cancer is a RelB-associated cancer. In some embodiments, the *-associated cancer is a HOIL1-associated cancer. In some embodiments, the *-associated cancer is a NIK-associated cancer. In some embodiments, the *-associated cancer is a Regnase 1-associated cancer. In some embodiments, the *-associated cancer is a LIMA1α-associated cancer. In some embodiments, the *-associated cancer is a roquin-1-associated cancer. In some embodiments, the *-associated cancer is a TRAF6-associated cancer. In some embodiments, the *-associated cancer is a TAK1-associated cancer. In some embodiments, the *-associated cancer is a TAB1-associated cancer. In some embodiments, the *-associated cancer is a TAB2-associated cancer. In some embodiments, the *-associated cancer is a TAB3-associated cancer. In some embodiments, the *-associated cancer is a MKK7-associated cancer, and an IKKα-associated cancer. In some embodiments, the *-associated cancer is an IKKβ-associated cancer. In some embodiments, the *-associated cancer is an IKKγ-associated cancer. In some embodiments, the *-associated cancer is an IkBα-associated cancer. In some embodiments, the *-associated cancer is a p50-associated cancer. In some embodiments, the *-associated cancer is a p65-associated cancer. In some embodiments, the *-associated cancer is a c-Rel-associated cancer. In some embodiments, the *-associated cancer is a JNK1-associated cancer. In some embodiments, the *-associated cancer is a JNK2-associated cancer. In some embodiments, the *-associated cancer is a JNK3-associated cancer. In some embodiments, the *-associated cancer is a AP-1 transcription factor-associated cancer. In some embodiments, the *-associated cancer is a MYD88 transcription factor-associated cancer.

The phrase “dysregulation of a * gene, a * protein, or the expression or activity or level of any of the same” (where * is a particular CBM complex pathway gene or protein, described herein) refers to a genetic mutation (e.g., a chromosomal translocation that results in the expression of a fusion protein including a * domain and a fusion partner, a mutation in a * gene that results in the expression of a * protein that includes a deletion of at least one amino acid as compared to a wild-type * protein, a mutation in a * gene that results in the expression of a * protein with one or more point mutations as compared to a wild-type * protein, a mutation in a * gene that results in the expression of a * protein with at least one inserted amino acid as compared to a wild-type * protein, a gene duplication that results in an increased level of * protein in a cell, or a mutation in a regulatory sequence (e.g., a promoter and/or enhancer) that results in an increased level of* protein in a cell), an alternative spliced version of a * mRNA that results in a * protein having a deletion of at least one amino acid in the * protein as compared to the wild-type * protein, or increased expression (e.g., increased levels) of a wild-type * protein in a mammalian cell due to aberrant cell signaling and/or dysregulated autocrine/paracrine signaling (e.g., as compared to a control non-cancerous cell). As a further example, an increased copy number of the * gene can result in overexpression of the * protein. For example, a dysregulation of a * gene, a * protein, or expression or activity, or level of any of the same, can be the result of a gene or chromosome translocation which results in the expression of a fusion protein that contains a first portion of *, and a second portion of a partner protein (i.e., that is not *). In some examples, dysregulation of a * gene, a * protein, or expression or activity or level of any of the same can be a result of a gene translocation of one * gene with another non-* gene. In some embodiments, the * gene, a * protein, or the expression or activity or level of any of the same is selected from the group consisting of: CD28, BCR, HER1, HER2, PKC−, PKCθ, MALT1, CARD11, CARD14, A20, CYLD, RelB, HOIL1, NIK, Regnase 1, LIMA1α, roquin-1, TRAF6, TAK1, TAB1, TAB2, TAB3, MKK7, IKKα, IKKβ, IKKγ, IkBα, p50, p65, c-Rel, JNK1, JNK2, JNK3, MYD88, and an AP-1 transcription factor. In some embodiments, the * gene or * protein is CD28. In some embodiments, the * gene or * protein is BCR. In some embodiments, the * gene or * protein is HER1. In some embodiments, the * gene or * protein is HER2. In some embodiments, the * gene or * protein is PKCβ. In some embodiments, the * gene or * protein is PKCθ. In some embodiments, the * gene or * protein is MALT1. In some embodiments, the * gene or * protein is CARD11. In some embodiments, the * gene or * protein is CARD14. In some embodiments, the * gene or * protein is A20. In some embodiments, the * gene or * protein is CYLD. In some embodiments, the * gene or * protein is RelB. In some embodiments, the * gene or * protein is HOIL1. In some embodiments, the * gene or * protein is NIK. In some embodiments, the * gene or * protein is Regnase 1. In some embodiments, the * gene or * protein is LIMA1α. In some embodiments, the * gene or * protein is roquin-1. In some embodiments, the * gene or * protein is TRAF6. In some embodiments, the * gene or * protein is TAK1. In some embodiments, the * gene or * protein is TAB1. In some embodiments, the * gene or * protein is TAB2. In some embodiments, the * gene or * protein is TAB3. In some embodiments, the * gene or * protein is MKK7. In some embodiments, the * gene or * protein is IKKα. In some embodiments, the * gene or * protein is IKKβ. In some embodiments, the * gene or * protein is IKKγ. In some embodiments, the * gene or * protein is IkBα. In some embodiments, the * gene or * protein is p50. In some embodiments, the * gene or * protein is p65. In some embodiments, the * gene or * protein is c-Rel. In some embodiments, the * gene or * protein is JNK1. In some embodiments, the * gene or * protein is JNK2. In some embodiments, the * gene or * protein is JNK3. In some embodiments, the * gene or * protein is MYD88 transcription factor. In some embodiments, the * gene or * protein is AP-1 transcription factor.

In some embodiments, dysregulation of a * gene, a * protein, or expression or activity, or level of any of the same, can be a mutation in a * gene that encodes a * protein that is constitutively active or has increased activity as compared to a protein encoded by a * gene that does not include the mutation. In some embodiments, an increased copy number of the * gene can result in overexpression of* protein. In some embodiments, the * gene, * protein, or expression or activity, or level of any of the same, is CD28. In some embodiments, the * gene, * protein, or expression or activity, or level of any of the same, is BCR. In some embodiments, the * gene, * protein, or expression or activity, or level of any of the same, is HER1. In some embodiments, the * gene, * protein, or expression or activity, or level of any of the same, is HER2. In some embodiments, the * gene, * protein, or expression or activity, or level of any of the same, is PKCβ. In some embodiments, the * gene, * protein, or expression or activity, or level of any of the same, is PKCθ. In some embodiments, the * gene, * protein, or expression or activity, or level of any of the same, is CARD14. In some embodiments, the * gene, * protein, or expression or activity, or level of any of the same, is CARD9. In some embodiments, the * gene, * protein, or expression or activity, or level of any of the same, is CARD10. In some embodiments, the * gene, * protein, or expression or activity, or level of any of the same, is CARD11. In some embodiments, the * gene, * protein, or expression or activity, or level of any of the same, is MALT1.

As another example, a dysregulation of an * gene, an * protein, or expression or activity, or level of any of the same, can be a mutation in an * gene that encodes an * protein that is constitutively inactive or has decreased activity as compared to a protein encoded by an * gene that does not include the mutation. In some embodiments, the * gene, * protein, or expression or activity, or level of any of the same, is A20. In some embodiments, the * gene, * protein, or expression or activity, or level of any of the same, is CYLD. In some embodiments, the * gene, * protein, or expression or activity, or level of any of the same, is RelB. In some embodiments, the * gene, * protein, or expression or activity, or level of any of the same, is HOIL1. In some embodiments, the * gene, * protein, or expression or activity, or level of any of the same, is NIK.

Diseases or disorders “associated” with a particular gene or protein described herein refer to diseases or disorder associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such diseases or disorders are described herein. Likewise, cancers “associated” with a particular gene or protein described herein refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

Exemplary sequences of the proteins described herein are shown below.

An exemplary sequence of human CD28 is shown below:

(UniParc Accession No. UPI0000043F4D)
SEQ ID NO: 1
MLRLLLALNLFPSIQVTGNKILVKQSPMLVAYDNAVNLSCKYSYNLFSRE
FRASLHKGLDSAVEVCVVYGNYSQQLQVYSKTGENCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS

Non-limiting examples of dysregulation of a CD28 gene or a CD28 protein can be found in, for example, Rohr, et al., Leukemia 30.5 (2016): 1062-1070, Yoo, et al., Haematologica 101.6 (2016): 757-763, and Lee, et al., Haematologica 100.12 (2015): e505.

An exemplary sequence of human BCR is shown below:

(UniParc Accession No. UPI000016A088)
SEQ ID NO: 2
MVDPVGFAEAWKAQFPDSEPPRMELRSVGDIEQELERCKASIRRLEQEVN
QERFRMIYLQTLLAKEKKSYDRQRWGFRRAAQAPDGASEPRASASRPQPA
PADGADPPPAEEPEARPDGEGSPGKARPGTARRPGAAASGERDDRGPPAS
VAALRSNFERIRKGHGQPGADAEKPFYVNVEFHHERGLVKVNDKEVSDRI
SSLGSQAMQMERKKSQHGAGSSVGDASRPPYRGRSSESSCGVDGDYEDAE
LNPRFLKDNLIDANGGSRPPWPPLEYQPYQSIYVGGMMEGEGKGPLLRSQ
STSEQEKRLTWPRRSYSPRSFEDCGGGYTPDCSSNENLTSSEEDFSSGQS
SRVSPSPTTYRMFRDKSRSPSQNSQQSFDSSSPPTPQCHKRHRHCPVVVS
EATIVGVRKTGQIWPNDGEGAFHGDADGSFGTPPGYGCAADRAEEQRRHQ
DGLPYIDDSPSSSPHLSSKGRGSRDALVSGALESTKASELDLEKGLEMRK
WVLSGILASEETYLSHLEALLLPMKPLKAAATTSQPVLTSQQIETIFFKV
PELYEIHKEFYDGLFPRVQQWSHQQRVGDLFQKLASQLGVYRAFVDNYGV
AMEMAEKCCQANAQFAEISENLRARSNKDAKDPTTKNSLETLLYKPVDRV
TRSTLVLHDLLKHTPASHPDHPLLQDALRISQNFLSSINEEITPRRQSMT
VKKGEHRQLLKDSFMVELVEGARKLRHVFLFTDLLLCTKLKKQSGGKTQQ
YDCKWYIPLTDLSFQMVDELEAVPNIPLVPDEELDALKIKISQIKNDIQR
EKRANKGSKATERLKKKLSEQESLLLLMSPSMAFRVHSRNGKSYTFLISS
DYERAEWRENIREQQKKCFRSFSLTSVELQMLTNSCVKLQTVHSIPLTIN
KEDDESPGLYGELNVIVHSATGFKQSSNLYCTLEVDSFGYFVNKAKTRVY
RDTAEPNWNEEFEIELEGSQTLRILCYEKCYNKTKIPKEDGESTDRLMGK
GQVQLDPQALQDRDWQRTVIAMNGIEVKLSVKFNSREFSLKRMPSRKQTG
VFGVKIAVVTKRERSKVPYIVRQCVEEIERRGMEEVGIYRVSGVATDIQA
LKAAFDVNNKDVSVMMSEMDVNAIAGTLKLYFRELPEPLFTDEFYPNFAE
GIALSDPVAKESCMLNLLLSLPEANLLTFLFLLDHLKRVAEKEAVNKMSL
HNLATVFGPTLLRPSEKESKLPANPSQPITMTDSWSLEVMSQVQVLLYFL
QLEAIPAPDSKRQSILFSTEV

Non-limiting examples of dysregulation of a BCR gene or a BCR protein (e.g., a BCR-ABL fusion) can be found in, for example, Yang and Fu, Crit. Rev. Oncol./Hematol. 93.3 (2015): 277-292, Weisberg, et al. Nat. Rev. Cancer 7.5 (2007): 345-356, and Jabbour, et al. Cancer 117.9 (2011): 1800-1811.

An exemplary sequence of human HER1 is shown below:

(UniParc Accession No. UPI000003E750)
SEQ ID NO: 3
MRPSGTAGAALLALLAALCPASRALEEKKVCQGTSNKLTQLGTFEDHELS
LQRMENNCEVVLGNLEITYVQRNYDLSFLKTIQEVAGYVLIALNTVERIP
LENLQIIRGNMYYENSYALAVLSNYDANKTGLKELPMRNLQEILHGAVRF
SNNPALCNVESIQWRDIVSSDELSNMSMDFQNHLGSCQKCDPSCPNGSCW
GAGEENCQKLTKIICAQQCSGRCRGKSPSDCCHNQCAAGCTGPRESDCLV
CRKFRDEATCKDTCPPLMLYNPTTYQMDVNPEGKYSFGATCVKKCPRNYV
VTDHGSCVRACGADSYEMEEDGVRKCKKCEGPCRKVCNGIGIGEFKDSLS
INATNIKHFKNCTSISGDLHILPVAFRGDSFTHTPPLDPQELDILKTVKE
ITGELLIQAWPENRTDLHAFENLEIIRGRTKQHGQFSLAVVSLNITSLGL
RSLKEISDGDVIISGNKNLCYANTINWKKLFGTSGQKTKIISNRGENSCK
ATGQVCHALCSPEGCWGPEPRDCVSCRNVSRGRECVDKCNLLEGEPREFV
ENSECIQCHPECLPQAMNITCTGRGPDNCIQCAHYIDGPHCVKTCPAGVM
GENNTLVWKYADAGHVCHLCHPNCTYGCTGPGLEGCPTNGPKIPSIATGM
VGALLLLLVVALGIGLFMRRRHIVRKRTLRRLLQERELVEPLTPSGEAPN
QALLRILKETEFKKIKVLGSGAFGTVYKGLWIPEGEKVKIPVAIKELREA
TSPKANKEILDEAYVMASVDNPHVCRLLGICLTSTVQLITQLMPFGCLLD
YVREHKDNIGSQYLLNWCVQIAKGMNYLEDRRLVHRDLAARNVLVKTPQH
VKITDEGLAKLLGAEEKEYHAEGGKVPIKWMALESILHRIYTHQSDVWSY
GVTVWELMTFGSKPYDGIPASEISSILEKGERLPQPPICTIDVYMIMVKC
WMIDADSRPKFRELIIEFSKMARDPQRYLVIQGDERMHLPSPTDSNFYRA
LMDEEDMDDVVDADEYLIPQQGFFSSPSTSRTPLLSSLSATSNNSTVACI
DRNGLQSCPIKEDSFLQRYSSDPTGALTEDSIDDTFLPVPEYINQSVPKR
PAGSVQNPVYHNQPLNPAPSRDPHYQDPHSTAVGNPEYLNTVQPTCVNST
FDSPAHWAQKGSHQISLDNPDYQQDFFPKEAKPNGIFKGSTAENAEYLRV
APQSSEFIGA

Non-limiting examples of dysregulation of a HER1 gene or a HER1 protein can be found in, for example, Zhang, et al., Oncotarget 7.48 (2016): 78985, Ellison, et al., Journal of Clinical Pathology 66.2 (2013): 79-89, Midha, et al., American Journal of Cancer Research 5.9 (2015): 2892, and YaaGIoto, et al., Lung Cancer 63.3 (2009): 315-321.

An exemplary sequence of human HER2 is shown below:

(UniParc Accession No. UPI000003F55F)
SEQ ID NO: 4
MELAALCRWGLLLALLPPGAASTQVCTGTDMKLRLPASPETHLDMLRHLY
QGCQVVQGNLELTYLPTNASLSFLQDIQEVQGYVLIAHNQVRQVPLQRLR
IVRGTQLFEDNYALAVLDNGDPLNNTTPVTGASPGGLRELQLRSLTEILK
GGVLIQRNPQLCYQDTILWKDIFHKNNQLALTLIDTNRSRACHPCSPMCK
GSRCWGESSEDCQSLTRTVCAGGCARCKGPLPTDCCHEQCAAGCTGPKHS
DCLACLHENHSGICELHCPALVTYNTDTFESMPNPEGRYTFGASCVTACP
YNYLSTDVGSCTLVCPLHNQEVTAEDGTQRCEKCSKPCARVCYGLGMEHL
REVRAVTSANIQEFAGCKKIFGSLAFLPESFDGDPASNTAPLQPEQLQVF
ETLEEITGYLYISAWPDSLPDLSVFQNLQVIRGRILHNGAYSLTLQGLGI
SWLGLRSLRELGSGLALIHHNTHLCFVHTVPWDQLFRNPHQALLHTANRP
EDECVGEGLACHQLCARGHCWGPGPTQCVNCSQFLRGQECVEECRVLQGL
PREYVNARHCLPCHPECQPQNGSVTCFGPEADQCVACAHYKDPPFCVARC
PSGVKPDLSYMPIWKFPDEEGACQPCPINCTHSCVDLDDKGCPAEQRASP
LTSIISAVVGILLVVVLGVVFGILIKRRQQKIRKYTMRRLLQETELVEPL
TPSGAMPNQAQMRILKETELRKVKVLGSGAFGTVYKGIWIPDGENVKIPV
AIKVLRENTSPKANKEILDEAYVMAGVGSPYVSRLLGICLTSTVQLVTQL
MPYGCLLDHVRENRGRLGSQDLLNWCMQIAKGMSYLEDVRLVHRDLAARN
VLVKSPNHVKITDFGLARLLDIDETEYHADGGKVPIKWMALESILRRRFT
HQSDVWSYGVTVWELMTFGAKPYDGIPAREIPDLLEKGERLPQPPICTID
VYMIMVKCWMIDSECRPRFRELVSEFSRMARDPQRFVVIQNEDLGPASPL
DSTFYRSLLEDDDMGDLVDAEEYLVPQQGFFCPDPAPGAGGMVHHRHRSS
STRSGGGDLTLGLEPSEEEAPRSPLAPSEGAGSDVFDGDLGMGAAKGLQS
LPTHDPSPLQRYSEDPTVPLPSETDGYVAPLTCSPQPEYVNQPDVRPQPP
SPREGPLPAARPAGATLERPKTLSPGKNGVVKDVFAFGGAVENPEYLTPQ
GGAAPQPHPPPAFSPAFDNLYYWDQDPPERGAPPSTFKGTPTAENPEYLG
LDVPV

Non-limiting examples of dysregulation of a HER2 gene or a HER2 protein can be found, for example, Petrelli, Fausto, et al., Breast Cancer Research and Treatment 166.2 (2017): 339-349, Yan, et al., Cancer and Metastasis Reviews 34.1 (2015): 157-164, Koshkin, et al., Bladder Cancer 5.1 (2019): 1-12, and Connell, et al., ESMO Open 2.5 (2017).

The term “cancer associated with a signal transducer between a cell surface receptor and a CBM complex” as used herein refers to cancers associated with or having a dysregulation of a gene, a protein, or the expression or activity or level of any (e.g., one or more) of the same associated with a signal transducer between a cell surface receptor and a CBM complex. In some embodiments, a cancer associated with a signal transducer between a cell surface receptor and a CBM complex is selected from the group consisting of a PKCβ-associated cancer, PCKθ-associated cancer, and a combination thereof. The cancers “associated” with a particular gene or protein described in this paragraph refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

An exemplary sequence of human PKCβ is shown below:

(UniParc Accession No. UPI000012DF67)
SEQ ID NO: 5
MADPAAGPPPSEGEESTVRFARKGALRQKNVHEVKNHKFTARFFKQPTFC
SHCTDFIWGFGKQGFQCQVCCFVVHKRCHEFVTFSCPGADKGPASDDPRS
KHKFKIHTYSSPTFCDHCGSLLYGLIHQGMKCDTCMMNVHKRCVMNVPSL
CGTDHTERRGRIYIQAHIDRDVLIVLVRDAKNLVPMDPNGLSDPYVKLKL
IPDPKSESKQKTKTIKCSLNPEWNETFRFQLKESDKDRRLSVEIWDWDLT
SRNDFMGSLSFGISELQKASVDGWFKLLSQEEGEYFNVPVPPEGSEANEE
LRQKFERAKISQGTKVPEEKTTNTVSKFDNNGNRDRMKLTDFNFLMVLGK
GSFGKVMLSERKGTDELYAVKILKKDVVIQDDDVECTMVEKRVLALPGKP
PFLTQLHSCFQTMDRLYFVMEYVNGGDLMYHIQQVGRFKEPHAVFYAAEI
AIGLFFLQSKGIIYRDLKLDNVMLDSEGHIKIADFGMCKENIWDGVTTKT
FCGTPDYIAPEIIAYQPYGKSVDWWAFGVLLYEMLAGQAPFEGEDEDELF
QSIMEHNVAYPKSMSKEAVAICKGLMTKHPGKRLGCGPEGERDIKEHAFF
RYIDWEKLERKEIQPPYKPKARDKRDTSNEDKEFTRQPVELTPTDKLFIM
NLDQNEFAGFSYTNPEFVINV

An exemplary sequence of human PKCθ is shown below:

(UniParc Accession No. UPI000012DF74)
SEQ ID NO: 6
MSPFLRIGLSNFDCGSCQSCQGEAVNPYCAVLVKEYVESENGQMYIQKKP
TMYPPWDSTFDAHINKGRVMQIIVKGKNVDLISETTVELYSLAERCRKNN
GKTEIWLELKPQGRMLMNARYFLEMSDTKDMNEFETEGFFALHQRRGAIK
QAKVHHVKCHEFTATFFPQPTFCSVCHEFVWGLNKQGYQCRQCNAAIHKK
CIDKVIAKCTGSAINSRETMFHKERFKIDMPHRFKVYNYKSPTFCEHCGT
LLWGLARQGLKCDACGMNVHHRCQTKVANLCGINQKLMAEALAMIESTQQ
ARCLRDTEQIFREGPVEIGLPCSIKNEARPPCLPTPGKREPQGISWESPL
DEVDKMCHLPEPELNKERPSLQIKLKIEDFILHKMLGKGSFGKVFLAEFK
KTNQFFAIKALKKDVVLMDDDVECTMVEKRVLSLAWEHPFLTHMFCTFQT
KENLFFVMEYLNGGDLMYHIQSCHKEDLSRATFYAAEIILGLQFLHSKGI
VYRDLKLDNILLDKDGHIKIADFGMCKENMLGDAKTNTFCGTPDYIAPEI
LLGQKYNHSVDWWSFGVLLYEMLIGQSPFHGQDEEELFHSIRMDNPFYPR
WLEKEAKDLLVKLFVREPEKRLGVRGDIRQHPLFREINWEELERKEIDPP
FRPKVKSPFDCSNFDKEFLNEKPRLSFADRALINSMDQNMERNESFMNPG
MERLIS

The term “component of a CBM complex-associated cancer” as used herein refers to cancers associated with or having a dysregulation of a gene, a protein, or the expression or activity or level of any (e.g., one or more) of the same associated with a component of a CBM complex. In some embodiments, a component of a CBM complex-associated cancer is selected from the group consisting of a MALT1-associated cancer, a CARD11-associated cancer, a CARD14-associated cancer, a CARD10-associated cancer, a CARD9-associated cancer, a BCL10-associated cancer, and combinations thereof. In some embodiments, a CBM complex-associated cancer is selected from the group consisting of a MALT1-associated cancer, a CARD11-associated cancer, a BCL10-associated cancer, and combinations thereof. The cancers “associated” with a particular gene or protein described in this paragraph refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

The term “MALT1-associated autoimmune disorder” as used herein refers to autoimmune disorders associated with or having a dysregulation of a MALT1 gene, a MALT1 protein (also called herein MALT1 protease protein or MALT1 protease), or the expression or activity or level of any (e.g., one or more) of the same (e.g., any of the types of dysregulation of a MALT1 gene, a MALT1 protease, a MALT1 protease domain, or the expression or activity or level of any of the same described herein). Non-limiting examples of a MALT1-associated autoimmune disorders are described herein.

The term “MALT1-associated inflammatory disorder” as used herein refers to inflammatory disorders associated with or having a dysregulation of a MALT1 gene, a MALT1 protein (also called herein MALT1 protease protein or MALT1 protease), or the expression or activity or level of any (e.g., one or more) of the same (e.g., any of the types of dysregulation of a MALT1 gene, a MALT1 protease, a MALT1 protease domain, or the expression or activity or level of any of the same described herein). Non-limiting examples of a MALT1-associated inflammatory disorders are described herein.

The term “MALT1-associated cancer” as used herein refers to cancers associated with or having a dysregulation of a MALT1 gene, a MALT1 protein (also called herein MALT1 protease protein or MALT1 protease), or the expression or activity or level of any (e.g., one or more) of the same (e.g., any of the types of dysregulation of a MALT1 gene, a MALT1 protein, a MALT1 protease domain, or the expression or activity or level of any of the same described herein). Non-limiting examples of a MALT1-associated cancer are described herein.

The phrase “dysregulation of a MALT1 gene, a MALT1 protein, or the expression or activity or level of any of the same” refers to a genetic mutation (e.g., a chromosomal translocation that results in the expression of a fusion protein including a MALT1 protease domain and a fusion partner, a mutation in a MALT1 gene that results in the expression of a MALT1 protein that includes a deletion of at least one amino acid as compared to a wild-type MALT1 protein, a mutation in a MALT1 gene that results in the expression of a MALT1 protein with one or more point mutations as compared to a wild-type MALT1 protein, a mutation in a MALT1 gene that results in the expression of a MALT1 protein with at least one inserted amino acid as compared to a wild-type MALT1 protein, a gene duplication that results in an increased level of MALT1 protein in a cell, or a mutation in a regulatory sequence (e.g., a promoter and/or enhancer) that results in an increased level of MALT1 protein in a cell), an alternative spliced version of a MALT1 mRNA that results in a MALT1 protein having a deletion of at least one amino acid in the MALT1 protein as compared to the wild-type MALT1 protein, or increased expression (e.g., increased levels) of a wild-type MALT1 protein in a mammalian cell due to aberrant cell signaling and/or dysregulated autocrine/paracrine signaling (e.g., as compared to a control non-cancerous cell). As another example, a dysregulation of a MALT1 gene, a MALT1 protein, or expression or activity, or level of any of the same, can be a mutation in a MALT1 gene that encodes a MALT1 protein that is constitutively active or has increased activity as compared to a protein encoded by a MALT1 gene that does not include the mutation. As a further example, an increased copy number of the MALT1 gene can result in overexpression of MALT1 protease. For example, a dysregulation of a MALT1 gene, a MALT1 protein, or expression or activity, or level of any of the same, can be the result of a gene or chromosome translocation which results in the expression of a fusion protein that contains a first portion of MALT1 that includes a functional protease domain, and a second portion of a partner protein (i.e., that is not MALT1). In some examples, dysregulation of a MALT1 gene, a MALT1 protein, or expression or activity or level of any of the same can be a result of a gene translocation of one MALT1 gene with another non-MALT1 gene.

An exemplary sequence of human MALT1 is shown below:

SEQ ID NO: 7
(UniParc Accession No. UPI000004D05E)
MSLLGDPLQALPPSAAPTGPLLAPPAGATLNRLREPLLRRLSELLDQAPE
GRGWRRLAELAGSRGRLRLSCLDLEQCSLKVLEPEGSPSLCLLKLMGEKG
CTVTELSDFLQAMEHTEVLQLLSPPGIKITVNPESKAVLAGQFVKLCCRA
TGHPFVQYQWFKMNKEIPNGNTSELIFNAVHVKDAGFYVCRVNNNFTFEF
SQWSQLDVCDIPESFQRSVDGVSESKLQICVEPTSQKLMPGSTLVLQCVA
VGSPIPHYQWFKNELPLTHETKKLYMVPYVDLEHQGTYWCHVYNDRDSQD
SKKVEIIIGRTDEAVECTEDELNNLGHPDNKEQTTDQPLAKDKVALLIGN
MNYREHPKLKAPLVDVYELTNLLRQLDFKVVSLLDLTEYEMRNAVDEFLL
LLDKGVYGLLYYAGHGYENFGNSFMVPVDAPNPYRSENCLCVQNILKLMQ
EKETGLNVFLLDMCRKRNDYDDTIPILDALKVTANIVFGYATCQGAEAFE
IQHSGLANGIFMKFLKDRLLEDKKITVLLDEVAEDMGKCHLTKGKQALEI
RSSLSEKRALTDPIQGTEYSAESLVRNLQWAKAHELPESMCLKFDCGVQI
QLGFAAEFSNVMIIYTSIVYKPPEIIMCDAYVTDFPLDLDIDPKDANKGT
PEETGSYLVSKDLPKHCLYTRLSSLQKLKEHLVFTVCLSYQYSGLEDTVE
DKQEVNVGKPLIAKLDMHRGLGRKTCFQTCLMSNGPYQSSAATSGGAGHY
HSLQDPFHGVYHSHPGNPSNVTPADSCHCSRTPDAFISSFAHHASCHFSR
SNVPVETTDEIPFSFSDRLRISEK

Non-limiting examples of dysregulation of a MALT1 gene or a MALT1 protein are shown in Table B1 below.

TABLE B1
MALT1 Protein Amino Acid Substitutions/Insertions/Deletions

| | Non-limiting | Non-Limiting |
|---|---|---|
| | Exemplary | Exemplary MALT1- |
| Amino Acid Position(s) | Mutations | associated Cancers |
| 717 | M717I⁴ | |

MALT Fusion Partners

| | Non-limiting Exemplary |
|---|---|
| Fusion Partner | MALT1-Associated Cancer(s) |
| BIRC3 (Also called IAP2; | Diffuse Large B-cell Lymphoma (DLBCL)¹; |
| CIAP2; and API2)¹ | Extra nodal low-grade MALT lymphoma² |
| IGH | ABC-DLBCL² |
| SEC11C | Breast Cancer³ |

¹United States Patent US 10,711,036
²United States Patent Application Publication US20190160045A1
³United States Patent Application Publication US20130096021A1
⁴United States Patent Application Publication US20150320754A1

The term “CARD11-associated autoimmune disorder” as used herein refers to autoimmune disorders associated with or having a dysregulation of a CARD11 gene, a CARD11 protein, or the expression or activity or level of any (e.g., one or more) of the same (e.g., any of the types of dysregulation of a CARD11 gene, a CARD11 protein, or the expression or activity or level of any of the same described herein).

The term “CARD11-associated inflammatory disorder” as used herein refers to inflammatory disorders associated with or having a dysregulation of a CARD11 gene, a CARD11 protein, or the expression or activity or level of any (e.g., one or more) of the same (e.g., any of the types of dysregulation of a CARD11 gene, a CARD11 protein, or the expression or activity or level of any of the same described herein).

The term “CARD11-associated cancer” as used herein refers to cancers associated with or having a dysregulation of a CARD11 gene, a CARD11 protein, or the expression or activity or level of any (e.g., one or more) of the same (e.g., any of the types of dysregulation of a CARD11 gene, a CARD11 protein, or the expression or activity or level of any of the same described herein). Non-limiting examples of a CARD11-associated cancer are described herein.

The phrase “dysregulation of a CARD11 gene, a CARD11 protein, or the expression or activity or level of any of the same” refers to a genetic mutation (e.g., a chromosomal translocation that results in the expression of a fusion protein including a CARD11 domain and a fusion partner, a mutation in a CARD11 gene that results in the expression of a CARD11 protein that includes a deletion of at least one amino acid as compared to a wild-type CARD11 protein, a mutation in a CARD11 gene that results in the expression of a CARD11 protein with one or more point mutations as compared to a wild-type CARD11 protein, a mutation in a CARD11 gene that results in the expression of a CARD11 protein with at least one inserted amino acid as compared to a wild-type CARD11 protein, a gene duplication that results in an increased level of CARD11 protein in a cell, or a mutation in a regulatory sequence (e.g., a promoter and/or enhancer) that results in an increased level of CARD11 protein in a cell), an alternative spliced version of a CARD11 mRNA that results in a CARD11 protein having a deletion of at least one amino acid in the CARD11 protein as compared to the wild-type CARD11 protein, or increased expression (e.g., increased levels) of a wild-type CARD11 protein in a mammalian cell due to aberrant cell signaling and/or dysregulated autocrine/paracrine signaling (e.g., as compared to a control non-cancerous cell). As another example, a dysregulation of a CARD11 gene, a CARD11 protein, or expression or activity, or level of any of the same, can be a mutation in a CARD11 gene that encodes a CARD11 protein that is constitutively active or has increased activity as compared to a protein encoded by a CARD11 gene that does not include the mutation. As a further example, an increased copy number of the CARD11 gene can result in overexpression of CARD11 protein. For example, a dysregulation of a CARD11 gene, a CARD11 protein, or expression or activity, or level of any of the same, can be the result of a gene or chromosome translocation which results in the expression of a fusion protein that contains a first portion of CARD11, and a second portion of a partner protein (i.e., that is not CARD11). In some examples, dysregulation of a CARD11 gene, a CARD11 protein, or expression or activity or level of any of the same can be a result of a gene translocation of one CARD11 gene with another non-CARD11 gene.

An exemplary sequence of human CARD11 is shown below:

SEQ ID NO: 8
(UniParc Accession No. UPI00003FED38)
MPGGGPEMDDYMETLKDEEDALWENVECNRHMLSRYINPAKLTPYLRQCK
VIDEQDEDEVLNAPMLPSKINRAGRLLDILHTKGQRGYVVFLESLEFYYP
ELYKLVTGKEPTRRFSTIVVEEGHEGLTHFLMNEVIKLQQQMKAKDLQRC
ELLARLRQLEDEKKQMTLTRVELLTFQERYYKMKEERDSYNDELVKVKDD
NYNLAMRYAQLSEEKNMAVMRSRDLQLEIDQLKHRLNKMEEECKLERNQS
LKLKNDIENRPKKEQVLELERENEMLKTKNQELQSIIQAGKRSLPDSDKA
ILDILEHDRKEALEDRQELVNRIYNLQEEARQAEELRDKYLEEKEDLELK
CSTLGKDCEMYKHRMNTVMLQLEEVERERDQAFHSRDEAQTQYSQCLIEK
DKYRKQIRELEEKNDEMRIEMVRREACIVNLESKLRRLSKDSNNLDQSLP
RNLPVTIISQDFGDASPRINGQEADDSSTSEESPEDSKYFLPYHPPQRRM
NLKGIQLQRAKSPISLKRTSDFQAKGHEEEGTDASPSSCGSLPITNSFTK
MQPPRSRSSIMSITAEPPGNDSIVRRYKEDAPHRSTVEEDNDSGGFDALD
LDDDSHERYSFGPSSIHSSSSSHQSEGLDAYDLEQVNLMFRKFSLERPFR
PSVTSVGHVRGPGPSVQHTTLNGDSLTSQLTLLGGNARGSFVHSVKPGSL
AEKAGLREGHQLLLLEGCIRGERQSVPLDTCTKEEAHWTIQRCSGPVTLH
YKVNHEGYRKLVKDMEDGLITSGDSFYIRLNLNISSQLDACTMSLKCDDV
VHVRDTMYQDRHEWLCARVDPFTDHDLDMGTIPSYSRAQQLLLVKLQRLM
HRGSREEVDGTHHTLRALRNTLQPEEALSTSDPRVSPRLSRASFLFGQLL
QFVSRSENKYKRMNSNERVRIISGSPLGSLARSSLDATKLLTEKQEELDP
ESELGKNLSLIPYSLVRAFYCERRRPVLFTPTVLAKTLVQRLLNSGGAME
FTICKSDIVTRDEFLRRQKTETIIYSREKNPNAFECIAPANIEAVAAKNK
HCLLEAGIGCTRDLIKSNIYPIVLFIRVCEKNIKRFRKLLPRPETEEEFL
RVCRLKEKELEALPCLYATVEPDMWGSVEELLRVVKDKIGEEQRKTIWVD
EDQL

Non-limiting examples of dysregulation of a CARD11 gene or a CARD11 protein are shown in Table B2 below.

TABLE B2
CARD11 Protein Amino Acid Substitutions/Insertions/Deletions

| Amino Acid | Non-limiting Exemplary | Non-Limiting Exemplary |
|---|---|---|
| Position(s) | Mutations | CARD11-associated Cancers |
| 47 | R47C² | Cutaneous squamous cell |
| | | carcinoma² |
| 123 | G123S¹ | Lymphoma¹ |
| 126 | G126D¹ | Lymphoma¹ |
| 130 | F130V² | Cutaneous squamous cell |
| | | carcinoma² |
| 167 | T167M² | Cutaneous squamous cell |
| | | carcinoma² |
| 215 | K215M, K215N¹ | Lymphoma¹ |
| 230 | D230N¹ | Lymphoma¹ |
| 357 | D357E¹ | Lymphoma¹ |
| 360 | M360V¹ | Lymphoma¹ |
| 361 | Y361C¹ | Lymphoma¹ |
| 368 | V368I² | Cutaneous squamous cell |
| | | carcinoma² |
| 737 | H737L² | Cutaneous squamous cell |
| | | carcinoma² |
| 750 | H750R² | Cutaneous squamous cell |
| | | carcinoma² |
| 833 | P833L² | Cutaneous squamous cell |
| | | carcinoma² |
| 900 | L900F² | Cutaneous squamous cell |
| | | carcinoma² |
| 1015 | L1015F² | Cutaneous squamous cell |
| | | carcinoma² |
| 1016 | R1016L² | Cutaneous squamous cell |
| | | carcinoma² |
| 1085 | R1085S² | Cutaneous squamous cell |
| | | carcinoma² |
| 1086 | F1086S² | Cutaneous squamous cell |
| | | carcinoma² |

¹Wu, et al., Oncotarget 7.25 (2016): 38180.
²Watt, et al. The American Journal of Pathology 185.9 (2015): 2354-2363.

The term “CARD14-associated autoimmune disorder” as used herein refers to autoimmune disorders associated with or having a dysregulation of a CARD14 gene, a CARD14 protein, or the expression or activity or level of any (e.g., one or more) of the same (e.g., any of the types of dysregulation of a CARD14 gene, a CARD14 protein, or the expression or activity or level of any of the same described herein).

The term “CARD14-associated inflammatory disorder” as used herein refers to inflammatory disorders associated with or having a dysregulation of a CARD14 gene, a CARD14 protein, or the expression or activity or level of any (e.g., one or more) of the same (e.g., any of the types of dysregulation of a CARD14 gene, a CARD14 protein, or the expression or activity or level of any of the same described herein).

The term “CARD14-associated cancer” as used herein refers to cancers associated with or having a dysregulation of a CARD14 gene, a CARD14 protein, or the expression or activity or level of any (e.g., one or more) of the same (e.g., any of the types of dysregulation of a CARD14 gene, a CARD14 protein, or the expression or activity or level of any of the same described herein).

An exemplary sequence of human CARD14 is shown below:

SEQ ID NO: 9
(UniParc Accession No. UPI000013D81B)
MGELCRRDSALTALDEETLWEMMESHRHRIVRCICPSRLTPYLRQAKVLC
QLDEEEVLHSPRLTNSAMRAGHLLDLLKTRGKNGAIAFLESLKFHNPDVY
TLVTGLQPDVDESNFSGLMETSKLTECLAGAIGSLQEELNQEKGQKEVLL
RRCQQLQEHLGLAETRAEGLHQLEADHSRMKREVSAHFHEVLRLKDEMLS
LSLHYSNALQEKELAASRCRSLQEELYLLKQELQRANMVSSCELELQEQS
LRTASDQESGDEELNRLKEENEKLRSLTFSLAEKDILEQSLDEARGSRQE
LVERIHSLRERAVAAERQREQYWEEKEQTLLQFQKSKMACQLYREKVNAL
QAQVCELQKERDQAYSARDSAQREISQSLVEKDSLRRQVFELTDQVCELR
TQLRQLQAEPPGVLKQEARTREPCPREKQRLVRMHAICPRDDSDCSLVSS
TESQLLSDLSATSSRELVDSFRSSSPAPPSQQSLYKRVAEDFGEEPWSFS
SCLEIPEGDPGALPGAKAGDPHLDYELLDTADLPQLESSLQPVSPGRLDV
SESGVLMRRRPARRILSQVTMLAFQGDALLEQISVIGGNLTGIFIHRVTP
GSAADQMALRPGTQIVMVDYEASEPLFKAVLEDTTLEEAVGLLRRVDGFC
CLSVKVNTDGYKRLLQDLEAKVATSGDSFYIRVNLAMEGRAKGELQVHCN
EVLHVTDTMFQGCGCWHAHRVNSYTMKDTAAHGTIPNYSRAQQQLIALIQ
DMTQQCTVTRKPSSGGPQKLVRIVSMDKAKASPLRLSFDRGQLDPSRMEG
SSTCFWAESCLTLVPYTLVRPHRPARPRPVLLVPRAVGKILSEKLCLLQG
FKKCLAEYLSQEEYEAWSQRGDIIQEGEVSGGRCWVTRHAVESLMEKNTH
ALLDVQLDSVCTLHRMDIFPIVIHVSVNEKMAKKLKKGLQRLGTSEEQLL
EAARQEEGDLDRAPCLYSSLAPDGWSDLDGLLSCVRQAIADEQKKVVWTE
QSPR

The term “CARD10-associated autoimmune disorder” as used herein refers to autoimmune disorders associated with or having a dysregulation of a CARD10 gene, a CARD10 protein, or the expression or activity or level of any (e.g., one or more) of the same (e.g., any of the types of dysregulation of a CARD10 gene, a CARD10 protein, or the expression or activity or level of any of the same described herein).

The term “CARD10-associated inflammatory disorder” as used herein refers to inflammatory disorders associated with or having a dysregulation of a CARD10 gene, a CARD10 protein, or the expression or activity or level of any (e.g., one or more) of the same (e.g., any of the types of dysregulation of a CARD10 gene, a CARD10 protein, or the expression or activity or level of any of the same described herein).

The term “CARD10-associated cancer” as used herein refers to cancers associated with or having a dysregulation of a CARD10 gene, a CARD10 protein, or the expression or activity or level of any (e.g., one or more) of the same (e.g., any of the types of dysregulation of a CARD10 gene, a CARD10 protein, or the expression or activity or level of any of the same described herein).

The phrase “dysregulation of a CARD10 gene, a CARD10 protein, or the expression or activity or level of any of the same” refers to a genetic mutation (e.g., a chromosomal translocation that results in the expression of a fusion protein including a CARD10 domain and a fusion partner, a mutation in a CARD10 gene that results in the expression of a CARD10 protein that includes a deletion of at least one amino acid as compared to a wild-type CARD10 protein, a mutation in a CARD10 gene that results in the expression of a CARD10 protein with one or more point mutations as compared to a wild-type CARD10 protein, a mutation in a CARD10 gene that results in the expression of a CARD10 protein with at least one inserted amino acid as compared to a wild-type CARD10 protein, a gene duplication that results in an increased level of CARD10 protein in a cell, or a mutation in a regulatory sequence (e.g., a promoter and/or enhancer) that results in an increased level of CARD10 protein in a cell), an alternative spliced version of a CARD10 mRNA that results in a CARD10 protein having a deletion of at least one amino acid in the CARD10 protein as compared to the wild-type CARD10 protein, or increased expression (e.g., increased levels) of a wild-type CARD10 protein in a mammalian cell due to aberrant cell signaling and/or dysregulated autocrine/paracrine signaling (e.g., as compared to a control non-cancerous cell). As another example, a dysregulation of a CARD10 gene, a CARD10 protein, or expression or activity, or level of any of the same, can be a mutation in a CARD10 gene that encodes a CARD10 protein that is constitutively active or has increased activity as compared to a protein encoded by a CARD10 gene that does not include the mutation. As a further example, an increased copy number of the CARD10 gene can result in overexpression of CARD10 protein. For example, a dysregulation of a CARD10 gene, a CARD10 protein, or expression or activity, or level of any of the same, can be the result of a gene or chromosome translocation which results in the expression of a fusion protein that contains a first portion of CARD10, and a second portion of a partner protein (i.e., that is not CARD10). In some examples, dysregulation of a CARD10 gene, a CARD10 protein, or expression or activity or level of any of the same can be a result of a gene translocation of one CARD10 gene with another non-CARD10 gene.

An exemplary sequence of human CARD10 is shown below:

SEQ ID NO: 10
(UniParc Accession No. UPI0000044645)
MPGRAEAGEAEEEAGAGSGSEAEEDALWERIEGVRHRLARALNPAKLTPY
LRQCRVIDEQDEEEVLSTYRFPCRVNRTGRLMDILRCRGKRGYEAFLEAL
EFYYPEHFTLLTGQEPAQRCSMILDEEGPEGLTQFLMTEVRRLREARKSQ
LQREQQLQARGRVLEEERAGLEQRLRDQQQAQERCQRLREDWEAGSLELL
RLKDENYMIAMRLAQLSEEKNSAVLRSRDLQLAVDQLKLKVSRLEEECAL
LRRARGPPPGAEEKEKEKEKEKEPDNVDLVSELRAENQRLTASLRELQEG
LQQEASRPGAPGSERILLDILEHDWREAQDSRQELCQKLHAVQGELQWAE
ELRDQYLQEMEDLRLKHRTLQKDCDLYKHRMATVLAQLEEIEKERDQAIQ
SRDRIQLQYSQSLIEKDQYRKQVRGLEAERDELLTTLTSLEGTKALLEVQ
LQRAQGGTCLKACASSHSLCSNLSSTWSLSEFPSPLGGPEATGEAAVMGG
PEPHNSEEATDSEKEINRLSILPFPPSAGSILRRQREEDPAPPKRSFSSM
SDITGSVTLKPWSPGLSSSSSSDSVWPLGKPEGLLARGCGLDFLNRSLAI
RVSGRSPPGGPEPQDKGPDGLSFYGDRWSGAVVRRVLSGPGSARMEPREQ
RVEAAGLEGACLEAEAQQRTLLWNQGSTLPSLMDSKACQSFHEALEAWAK
GPGAEPFYIRANLTLPERADPHALCVKAQEILRLVDSAYKRRQEWFCTRV
DPLTLRDLDRGTVPNYQRAQQLLEVQEKCLPSSRHRGPRSNLKKRALDQL
RLVRPKPVGAPAGDSPDQLLLEPCAEPERSLRPYSLVRPLLVSALRPVVL
LPECLAPRLIRNLLDLPSSRLDFQVCPAESLSGEELCPSSAPGAPKAQPA
TPGLGSRIRAIQESVGKKHCLLELGARGVRELVQNEIYPIVIHVEVTEKN
VREVRGLLGRPGWRDSELLRQCRGSEQVLWGLPCSWVQVPAHEWGHAEEL
AKVVRGRILQEQARLVWVECGSSRGCPSSSEA

The term “CARD9-associated autoimmune disorder” as used herein refers to autoimmune disorders associated with or having a dysregulation of a CARD9 gene, a CARD9 protein, or the expression or activity or level of any (e.g., one or more) of the same (e.g., any of the types of dysregulation of a CARD9 gene, a CARD9 protein, or the expression or activity or level of any of the same described herein).

The term “CARD9-associated inflammatory disorder” as used herein refers to inflammatory disorders associated with or having a dysregulation of a CARD9 gene, a CARD9 protein, or the expression or activity or level of any (e.g., one or more) of the same (e.g., any of the types of dysregulation of a CARD9 gene, a CARD9 protein, or the expression or activity or level of any of the same described herein).

The term “CARD9-associated cancer” as used herein refers to cancers associated with or having a dysregulation of a CARD9 gene, a CARD9 protein, or the expression or activity or level of any (e.g., one or more) of the same (e.g., any of the types of dysregulation of a CARD9 gene, a CARD9 protein, or the expression or activity or level of any of the same described herein).

The phrase “dysregulation of a CARD9 gene, a CARD9 protein, or the expression or activity or level of any of the same” refers to a genetic mutation (e.g., a chromosomal translocation that results in the expression of a fusion protein including a CARD9 domain and a fusion partner, a mutation in a CARD9 gene that results in the expression of a CARD9 protein that includes a deletion of at least one amino acid as compared to a wild-type CARD9 protein, a mutation in a CARD9 gene that results in the expression of a CARD9 protein with one or more point mutations as compared to a wild-type CARD9 protein, a mutation in a CARD9 gene that results in the expression of a CARD9 protein with at least one inserted amino acid as compared to a wild-type CARD9 protein, a gene duplication that results in an increased level of CARD9 protein in a cell, or a mutation in a regulatory sequence (e.g., a promoter and/or enhancer) that results in an increased level of CARD9 protein in a cell), an alternative spliced version of a CARD9 mRNA that results in a CARD9 protein having a deletion of at least one amino acid in the CARD9 protein as compared to the wild-type CARD9 protein, or increased expression (e.g., increased levels) of a wild-type CARD9 protein in a mammalian cell due to aberrant cell signaling and/or dysregulated autocrine/paracrine signaling (e.g., as compared to a control non-cancerous cell). As another example, a dysregulation of a CARD9 gene, a CARD9 protein, or expression or activity, or level of any of the same, can be a mutation in a CARD9 gene that encodes a CARD9 protein that is constitutively active or has increased activity as compared to a protein encoded by a CARD9 gene that does not include the mutation. As a further example, an increased copy number of the CARD9 gene can result in overexpression of CARD9 protein. For example, a dysregulation of a CARD9 gene, a CARD9 protein, or expression or activity, or level of any of the same, can be the result of a gene or chromosome translocation which results in the expression of a fusion protein that contains a first portion of CARD9, and a second portion of a partner protein (i.e., that is not CARD9). In some examples, dysregulation of a CARD9 gene, a CARD9 protein, or expression or activity or level of any of the same can be a result of a gene translocation of one CARD9 gene with another non-CARD9 gene.

An exemplary sequence of human CARD9 is shown below:

SEQ ID NO: 11
(UniParc Accession No. UPI000013E4EB)
MSDYENDDECWSVLEGFRVTLTSVIDPSRITPYLRQCKVLNPDDEEQVLS
DPNLVIRKRKVGVLLDILQRTGHKGYVAFLESLELYYPQLYKKVTGKEPA
RVFSMIIDASGESGLTQLLMTEVMKLQKKVQDLTALLSSKDDFIKELRVK
DSLLRKHQERVQRLKEECEAGSRELKRCKEENYDLAMRLAHQSEEKGAAL
MRNRDLQLEIDQLKHSLMKAEDDCKVERKHTLKLRHAMEQRPSQELLWEL
QQEKALLQARVQELEASVQEGKLDRSSPYIQVLEEDWRQALRDHQEQANT
IFSLRKDLRQGEARRLRCMEEKEMFELQCLALRKDSKMYKDRIEAILLQM
EEVAIERDQAIATREELHAQHARGLQEKDALRKQVRELGEKADELQLQVF
QCEAQLLAVEGRLRRQQLETLVLSSDLEDGSPRRSQELSLPQDLEDTQLS
DKGCLAGGGSPKQPFAALHQEQVLRNPHDAGLSSGEPPEKERRRLKESFE
NYRRKRALRKMQKGWRQGEEDRENTTGSDNTDTEGS

The term “BCL10-associated autoimmune disorder” as used herein refers to autoimmune disorders associated with or having a dysregulation of a BCL10 gene, a BCL10 protein, or the expression or activity or level of any (e.g., one or more) of the same (e.g., any of the types of dysregulation of a BCL10 gene, a BCL10 protein, or the expression or activity or level of any of the same described herein).

The term “BCL10-associated inflammatory disorder” as used herein refers to inflammatory disorders associated with or having a dysregulation of a BCL10 gene, a BCL10 protein, or the expression or activity or level of any (e.g., one or more) of the same (e.g., any of the types of dysregulation of a BCL10 gene, a BCL10 protein, or the expression or activity or level of any of the same described herein).

The term “BCL10-associated cancer” as used herein refers to cancers associated with or having a dysregulation of a BCL10 gene, a BCL10 protein, or the expression or activity or level of any (e.g., one or more) of the same (e.g., any of the types of dysregulation of a BCL10 gene, a BCL10 protein, or the expression or activity or level of any of the same described herein).

The phrase “dysregulation of a BCL10 gene, a BCL10 protein, or the expression or activity or level of any of the same” refers to a genetic mutation (e.g., a chromosomal translocation that results in the expression of a fusion protein including a BCL10 domain and a fusion partner, a mutation in a BCL10 gene that results in the expression of a BCL10 protein that includes a deletion of at least one amino acid as compared to a wild-type BCL10 protein, a mutation in a BCL10 gene that results in the expression of a BCL10 protein with one or more point mutations as compared to a wild-type BCL10 protein, a mutation in a BCL10 gene that results in the expression of a BCL10 protein with at least one inserted amino acid as compared to a wild-type BCL10 protein, a gene duplication that results in an increased level of BCL10 protein in a cell, or a mutation in a regulatory sequence (e.g., a promoter and/or enhancer) that results in an increased level of BCL10 protein in a cell), an alternative spliced version of a BCL10 mRNA that results in a BCL10 protein having a deletion of at least one amino acid in the BCL10 protein as compared to the wild-type BCL10 protein, or increased expression (e.g., increased levels) of a wild-type BCL10 protein in a mammalian cell due to aberrant cell signaling and/or dysregulated autocrine/paracrine signaling (e.g., as compared to a control non-cancerous cell). For example, a dysregulation of a BCL10 gene, a BCL10 protein, or expression or activity, or level of any of the same, can be the result of a gene or chromosome translocation which results in the expression of a fusion protein that contains a first portion of BCL10, and a second portion of a partner protein (i.e., that is not BCL10). In some examples, dysregulation of a BCL10 gene, a BCL10 protein, or expression or activity or level of any of the same can be a result of a gene translocation of one BCL10 gene with another non-BCL10 gene.

An exemplary sequence of human BCL10 is shown below:

SEQ ID NO: 12
(UniParc Accession No. UPI000012682F)
MEPTAPSLTEEDLTEVKKDALENLRVYLCEKIIAERHFDHLRAKKILSRE
DTEEISCRTSSRKRAGKLLDYLQENPKGLDTLVESIRREKTQNFLIQKIT
DEVLKLRNIKLEHLKGLKCSSCEPFPDGATNNLSRSNSDESNFSEKLRAS
TVMYHPEGESSTTPFFSTNSSLNLPVLEVGRTENTIFSSTTLPRPGDPGA
PPLPPDLQLEEEGTCANSSEMFLPLRSRTVSRQ

Non-limiting examples of dysregulation of a BCL10 gene or a BCL10 protein are shown in Table B3 below.

TABLE B3
BCL10 Protein Amino Acid Substitutions/Insertions/Deletions

| Amino | Non-limiting | Non-Limiting |
|---|---|---|
| Acid | Exemplary | Exemplary BCL10- |
| Position(s) | Mutations | associated Cancers |
| 5 | A5S² | Lymphoma² |
| 16 | V16E² | Lymphoma² |
| 20 | A20T¹ | Germ cell tumor¹ |
| 31 | K31E | Lymphoma² |
| 32 | I32V¹ | Lymphoma¹ |
| 43 | A43*² | Lymphoma² |
| 46 | I46*¹ | T-ALL¹, colonic carcinoma¹ |
| 49 | R49G¹ | Lymphoma¹ |
| 52 | T52I¹ | Mesothelioma¹ |
| 55 | I55*¹ | Lymphoma¹ |
| 57 | C57R² | Lymphoma² |
| 58 | R58G¹, R58*¹ | Germ cell tumor¹ |
| 64 | R64K² | Lymphoma² |
| 77 | K77*¹ | Lymphoma¹ |
| 80 | D80N | Lymphoma¹ |
| 91 | T91*¹ | Germ cell tumor¹ |
| 100 | T100S¹ | Lymphoma¹ |
| 101 | D101E² | Lymphoma² |
| 115 | K115*¹ | Lymphoma¹ |
| 116-126 | Splice mutation¹ | Lymphoma¹ |
| 116-121 | Splice mutation² | Lymphoma² |
| 116-120 | Splice mutation¹ | Mesothelioma¹ |
| 133 | L133*¹ | Lymphoma¹ |
| 134 | S134P² | Lymphoma² |
| 137 | N137*¹ | Lymphoma¹ |
| 143 | F143*¹ | Lymphoma¹ |
| 152 | V152*² | Lymphoma² |
| 165 | F165*² | Lymphoma² |
| 167 | S167*¹ | Lymphoma¹ |
| 168 | T168A² | Lymphoma² |
| 170-180 | del S170-G180¹ | Lymphoma¹ |
| 175-181 | del P175-G180¹ | Lymphoma¹ |
| 210 | del 210¹ | Lymphoma¹ |
| 213 | G213E | Lymphoma² |
| 218 | S218F¹ | Germ cell tumor¹ |
| 230 | V230I² | Lymphoma² |
| Stop | Stop->R | Lymphoma² |

¹Willis, et al. Cell 96.1 (1999): 35-45.
²Zhang, et al. Nature Genetics 22.1 (1999): 63-68.

The term “MALT1 protease substrate-associated cancer” as used herein refers to cancers associated with or having a dysregulation of agene, a protein, or the expression or activity or level of any (e.g., one or more) of the same associated with a MALT1 protease substrate. In some embodiments, a MALT1 protease substrate-associated cancer is selected from the group consisting of a BCL10-associated cancer, an A20-associated cancer, a CYLD-associated cancer, a RelB-associated cancer, a Regnase 1-associated cancer, a roquin-1-associated cancer, a HOIL1-associated cancer, a NIK associated cancer, a LIMA1α-associated cancer, and combinations thereof. In some embodiments, a MALT1 protease substrate-associated cancer is selected from the group consisting of a BCL10-associated cancer, an A20-associated cancer, a CYLD-associated cancer, and combinations thereof. The cancers “associated” with a particular gene or protein described in this paragraph refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

An exemplary sequence of human A20 is shown below:

SEQ ID NO: 13
(UniParc Accession No. UPI000000D92D)
MAEQVLPQALYLSNMRKAVKIRERTPEDIFKPTNGIIHHFKTMHRYTLEM
FRTCQFCPQFREIIHKALIDRNIQATLESQKKLNWCREVRKLVALKTNGD
GNCLMHATSQYMWGVQDTDLVLRKALFSTLKETDTRNFKFRWQLESLKSQ
EFVETGLCYDTRNWNDEWDNLIKMASTDTPMARSGLQYNSLEEIHIFVLC
NILRRPIIVISDKMLRSLESGSNFAPLKVGGIYLPLHWPAQECYRYPIVL
GYDSHHFVPLVTLKDSGPEIRAVPLVNRDRGRFEDLKVHFLTDPENEMKE
KLLKEYLMVIEIPVQGWDHGTTHLINAAKLDEANLPKEINLVDDYFELVQ
HEYKKWQENSEQGRREGHAQNPMEPSVPQLSLMDVKCETPNCPFFMSVNT
QPLCHECSERRQKNQNKLPKLNSKPGPEGLPGMALGASRGEAYEPLAWNP
EESTGGPHSAPPTAPSPFLFSETTAMKCRSPGCPFTLNVQHNGFCERCHN
ARQLHASHAPDHTRHLDPGKCQACLQDVTRTFNGICSTCFKRTTAEASSS
LSTSLPPSCHQRSKSDPSRLVRSPSPHSCHRAGNDAPAGCLSQAARTPGD
RTGTSKCRKAGCVYFGTPENKGFCTLCFIEYRENKHFAAASGKVSPTASR
FQNTIPCLGRECGTLGSTMFEGYCQKCFIEAQNQRFHEAKRTEEQLRSSQ
RRDVPRTTQSTSRPKCARASCKNILACRSEELCMECQHPNQRMGPGAHRG
EPAPEDPPKQRCRAPACDHFGNAKCNGYCNECFQFKQMYG

Non-limiting examples of dysregulation of an A20 gene or an A20 protein are shown in Table B4 below.

TABLE B4
A20 Protein Amino Acid Substitutions/Insertions/Deletions

| Amino | Non-limiting | Non-Limiting Exemplary |
|---|---|---|
| Acid Position(s) | Exemplary Mutations | A20-associated Cancers |
| 100 | D100*² | Extranodal marginal zone |
| | | lymphoma² |
| 162 | R162*² | Nodal marginal zone |
| | | lymphoma² |
| 183 | R183X¹ | Lymphoma¹ |
| 271 | R271X¹ | Lymphoma¹ |
| 278 | R278*² | Nodal marginal zone |
| | | lymphoma² |
| 288 | V288*² | Splenic marginal zone |
| | | lymphoma² |
| 491 | H491*² | Nodal marginal zone |
| | | lymphoma² |
| 633 | E633*² | Extranodal marginal zone |
| | | lymphoma² |

¹Johansson et al. Oncotarget 7.38 (2016): 62627.
²Novak, et al. Blood 113.20 (2009): 4918-4921.

An exemplary sequence of human CYLD is shown below:

SEQ ID NO: 14
(UniParc Accession No. UPI0000073A15)
MSSGLWSQEKVTSPYWEERIFYLLLQECSVTDKQTQKLLKVPKGSIGQYI
QDRSVGHSRIPSAKGKKNQIGLKILEQPHAVLFVDEKDVVEINEKFTELL
LAITNCEERFSLFKNRNRLSKGLQIDVGCPVKVQLRSGEEKFPGVVRFRG
PLLAERTVSGIFFGVELLEEGRGQGFTDGVYQGKQLFQCDEDCGVFVALD
KLELIEDDDTALESDYAGPGDTMQVELPPLEINSRVSLKVGETIESGTVI
FCDVLPGKESLGYFVGVDMDNPIGNWDGRFDGVQLCSFACVESTILLHIN
DIIPALSESVTQERRPPKLAFMSRGVGDKGSSSHNKPKATGSTSDPGNRN
RSELFYTLNGSSVDSQPQSKSKNTWYIDEVAEDPAKSLTEISTDFDRSSP
PLQPPPVNSLTTENRFHSLPFSLTKMPNINGSIGHSPLSLSAQSVMEELN
TAPVQESPPLAMPPGNSHGLEVGSLAEVKENPPFYGVIRWIGQPPGLNEV
LAGLELEDECAGCTDGTFRGTRYFTCALKKALFVKLKSCRPDSRFASLQP
VSNQIERCNSLAFGGYLSEVVEENTPPKMEKEGLEIMIGKKKGIQGHYNS
CYLDSTLFCLFAFSSVLDTVLLRPKEKNDVEYYSETQELLRTEIVNPLRI
YGYVCATKIMKLRKILEKVEAASGFTSEEKDPEEFLNILFHHILRVEPLL
KIRSAGQKVQDCYFYQIFMEKNEKVGVPTIQQLLEWSFINSNLKFAEAPS
CLIIQMPRFGKDFKLFKKIFPSLELNITDLLEDTPRQCRICGGLAMYECR
ECYDDPDISAGKIKQFCKTCNTQVHLHPKRLNHKYNPVSLPKDLPDWDWR
HGCIPCQNMELFAVLCIETSHYVAFVKYGKDDSAWLFFDSMADRDGGQNG
FNIPQVTPCPEVGEYLKMSLEDLHSLDSRRIQGCARRLLCDAYMCMYQSP
TMSLYK

Non-limiting examples of dysregulation of a CYLD gene or a CYLD protein can be found, for example, in Massoumii, Future Oncology 7.2 (2011): 285-297, Alameda, J. P., et al., Oncogene 29.50 (2010): 6522-6532, Williams, et al., Modern Pathology (2020): 1-13, and Courtois and Gilmore. Oncogene 25.51 (2006): 6831-6843.

An exemplary sequence of human RelB is shown below:

SEQ ID NO: 15
(UniParc Accession No. UPI00000012B7)
MLRSGPASGPSVPTGRAMPSRRVARPPAAPELGALGSPDLSSLSLAVSRS
TDELEIIDEYIKENGFGLDGGQPGPGEGLPRLVSRGAASLSTVTLGPVAP
PATPPPWGCPLGRLVSPAPGPGPQPHLVITEQPKQRGMRFRYECEGRSAG
SILGESSTEASKTLPAIELRDCGGLREVEVTACLVWKDWPHRVHPHSLVG
KDCTDGICRVRLRPHVSPRHSFNNLGIQCVRKKEIEAAIERKIQLGIDPY
NAGSLKNHQEVDMNVVRICFQASYRDQQGQMRRMDPVLSEPVYDKKSTNT
SELRICRINKESGPCTGGEELYLLCDKVQKEDISVVFSRASWEGRADFSQ
ADVHRQIAIVFKTPPYEDLEIVEPVTVNVFLQRLTDGVCSEPLPFTYLPR
DHDSYGVDKKRKRGMPDVLGELNSSDPHGIESKRRKKKPAILDHFLPNHG
SGPFLPPSALLPDPDFFSGTVSLPGLEPPGGPDLLDDGFAYDPTAPTLFT
MLDLLPPAPPHASAVVCSGGAGAVVGETPGPEPLTLDSYQAPGPGDGGTA
SLVGSNMFPNHYREAAFGGGLLSPGPEAT

An exemplary sequence of human Regnase 1 is shown below:

SEQ ID NO: 16
(UniParc Accession No. UPI000004D30E)
MSGPCGEKPVLEASPTMSLWEFEDSHSRQGTPRPGQELAAEEASALELQM
KVDFFRKLGYSSTEIHSVLQKLGVQADTNTVLGELVKHGTATERERQTSP
DPCPQLPLVPRGGGTPKAPNLEPPLPEEEKEGSDLRPVVIDGSNVAMSHG
NKEVFSCRGILLAVNWFLERGHTDITVFVPSWRKEQPRPDVPITDQHILR
ELEKKKILVFTPSRRVGGKRVVCYDDRFIVKLAYESDGIVVSNDTYRDLQ
GERQEWKRFIEERLLMYSFVNDKFMPPDDPLGRHGPSLDNFLRKKPLTLE
HRKQPCPYGRKCTYGIKCRFFHPERPSCPQRSVADELRANALLSPPRAPS
KDKNGRRPSPSSQSSSLLTESEQCSLDGKKLGAQASPGSRQEGLTQTYAP
SGRSLAPSGGSGSSFGPTDWLPQTLDSLPYVSQDCLDSGIGSLESQMSEL
WGVRGGGPGEPGPPRAPYTGYSPYGSELPATAAFSAFGRAMGAGHFSVPA
DYPPAPPAFPPREYWSEPYPLPPPTSVLQEPPVQSPGAGRSPWGRAGSLA
KEQASVYTKLCGVFPPHLVEAVMGRFPQLLDPQQLAAEILSYKSQHPSE

An exemplary sequence of human roquin-1 is shown below:

SEQ ID NO: 17
(UniParc Accession No. UPI00001D7DA8)
MPVQAPQWTDFLSCPICTQTFDETIRKPISLGCGHTVCKMCLNKLHRKAC
PFDQTTINTDIELLPVNSALLQLVGAQVPEQQPITLCSGVEDTKHYEEAK
KCVEELALYLKPLSSARGVGLNSTTQSVLSRPMQRKLVTLVHCQLVEEEG
RIRAMRAARSLGERTVTELILQHQNPQQLSSNLWAAVRARGCQFLGPAMQ
EEALKLVLLALEDGSALSRKVLVLFVVQRLEPRFPQASKTSIGHVVQLLY
RASCFKVTKRDEDSSLMQLKEEFRTYEALRREHDSQIVQIAMEAGLRIAP
DQWSSLLYGDQSHKSHMQSIIDKLQTPASFAQSVQELTIALQRTGDPANL
NRLRPHLELLANIDPSPDAPPPTWEQLENGLVAVRTVVHGLVDYIQNHSK
KGADQQQPPQHSKYKTYMCRDMKQRGGCPRGASCTFAHSQEELEKFRKMN
KRLVPRRPLSASLGQLNEVGLPSAAILPDEGAVDLPSRKPPALPNGIVST
GNTVTQLIPRGTDPSYDSSLKPGKIDHLSSSAPGSPPDLLESVPKSISAL
PVNPHSIPPRGPADLPPMPVTKPLQMVPRGSQLYPAQQTDVYYQDPRGAA
PPFEPAPYQQGMYYTPPPQCVSRFVRPPPSAPEPAPPYLDHYPPYLQERV
VNSQYGTQPQQYPPIYPSHYDGRRVYPAPSYTREEIFRESPIPIEIPPAA
VPSYVPESRERYQQIESYYPVAPHPTQIRPSYLREPPYSRLPPPPQPHPS
LDELHRRRKEIMAQLEERKVISPPPFAPSPTLPPTFHPEEFLDEDLKVAG
KYKGNDYSQYSPWSCDTIGSYIGTKDAKPKDVVAAGSVEMMNVESKGMRD
QRLDLQRRAAETSDDDLIPFGDRPTVSRFGAISRTSKTIYQGAGPMQAMA
PQGAPTKSINISDYSPYGTHGGWGASPYSPHQNIPSQGHFSERERISMSE
VASHGKPLPSAEREQLRLELQQLNHQISQQTQLRGLEAVSNRLVLQREAN
TLAGQSQPPPPPPPKWPGMISSEQLSLELHQVEREIGKRTRELSMENQCS
LDMKSKLNTSKQAENGQPEPQNKVPAEDLTLTFSDVPNGSALTQENISLL
SNKTSSLNLSEDPEGGGDNNDSQRSGVTPSSAP

An exemplary sequence of human HOIL1 is shown below:

SEQ ID NO: 17
(UniParc Accession No. UPI000006F045)
MDEKTKKAEEMALSLTRAVAGGDEQVAMKCAIWLAEQRVPLSVQLKPEVS
PTQDIRLWVSVEDAQMHTVTIWLTVRPDMTVASLKDMVFLDYGFPPVLQQ
WVIGQRLARDQETLHSHGVRQNGDSAYLYLLSARNTSLNPQELQRERQLR
MLEDLGFKDLTLQPRGPLEPGPPKPGVPQEPGRGQPDAVPEPPPVGWQCP
GCTFINKPTRPGCEMCCRARPEAYQVPASYQPDEEERARLAGEEEALRQY
QQRKQQQQEGNYLQHVQLDQRSLVLNTEPAECPVCYSVLAPGEAVVLREC
LHTFCRECLQGTIRNSQEAEVSCPFIDNTYSCSGKLLEREIKALLTPEDY
QRFLDLGISIAENRSAFSYHCKTPDCKGWCFFEDDVNEFTCPVCFHVNCL
LCKAIHEQMNCKEYQEDLALRAQNDVAARQTTEMLKVMLQQGEAMRCPQC
QIVVQKKDGCDWIRCTVCHTEICWVTKGPRWGPGGPGDTSGGCRCRVNGI
PCHPSCQNCH

An exemplary sequence of human NIK is shown below:

SEQ ID NO: 18
(UniParc Accession No. UPI0000074220)
MAVMEMACPGAPGSAVGQQKELPKAKEKTPPLGKKQSSVYKLEAVEKSPV
FCGKWEILNDVITKGTAKEGSEAGPAAISIIAQAECENSQEFSPTFSERI
FIAGSKQYSQSESLDQIPNNVAHATEGKMARVCWKGKRRSKARKKRKKKS
SKSLAHAGVALAKPLPRTPEQESCTIPVQEDESPLGAPYVRNTPQFTKPL
KEPGLGQLCFKQLGEGLRPALPRSELHKLISPLQCLNHVWKLHHPQDGGP
LPLPTHPFPYSRLPHPFPFHPLQPWKPHPLESFLGKLACVDSQKPLPDPH
LSKLACVDSPKPLPGPHLEPSCLSRGAHEKFSVEEYLVHALQGSVSSGQA
HSLTSLAKTWAARGSRSREPSPKTEDNEGVLLTEKLKPVDYEYREEVHWA
THQLRLGRGSFGEVHRMEDKQTGFQCAVKKVRLEVFRAEELMACAGLTSP
RIVPLYGAVREGPWVNIFMELLEGGSLGQLVKEQGCLPEDRALYYLGQAL
EGLEYLHSRRILHGDVKADNVLLSSDGSHAALCDFGHAVCLQPDGLGKSL
LTGDYIPGTETHMAPEVVLGRSCDAKVDVWSSCCMMLHMLNGCHPWTQFF
RGPLCLKIASEPPPVREIPPSCAPLTAQAIQEGLRKEPIHRVSAAELGGK
VNRALQQVGGLKSPWRGEYKEPRHPPPNQANYHQTLHAQPRELSPRAPGP
RPAEETTGRAPKLQPPLPPEPPEPNKSPPLTLSKEESGMWEPLPLSSLEP
APARNPSSPERKATVPEQELQQLEIELFLNSLSQPFSLEEQEQILSCLSI
DSLSLSDDSEKNPSKASQSSRDTLSSGVHSWSSQAEARSSSWNMVLARGR
PTDTPSYFNGVKVQIQSLNGEHLHIREFHRVKVGDIATGISSQIPAAAFS
LVTKDGQPVRYDMEVPDSGIDLQCTLAPDGSFAWSWRVKHGQLENRP

An exemplary sequence of human LIMA1α is shown below:

SEQ ID NO: 19
(UniParc Accession No. UPI000002A906)
MENCLGESRHEVEKSEISENTDASGKIEKYNVPLNRLKMMFEKGEPTQTK
ILRAQSRSASGRKISENSYSLDDLEIGPGQLSSSTFDSEKNESRRNLELP
RLSETSIKDRMAKYQAAVSKQSSSTNYTNELKASGGEIKIHKMEQKENVP
PGPEVCITHQEGEKISANENSLAVRSTPAEDDSRDSQVKSEVQQPVHPKP
LSPDSRASSLSESSPPKAMKKFQAPARETCVECQKTVYPMERLLANQQVF
HISCFRCSYCNNKLSLGTYASLHGRIYCKPHFNQLFKSKGNYDEGFGHRP
HKDLWASKNENEEILERPAQLANARETPHSPGVEDAPIAKVGVLAASMEA
KASSQQEKEDKPAETKKLRIAWPPPTELGSSGSALEEGIKMSKPKWPPED
EISKPEVPEDVDLDLKKLRRSSSLKERSRPFTVAASFQSTSVKSPKTVSP
PIRKGWSMSEQSEESVGGRVAERKQVENAKASKKNGNVGKTTWQNKESKG
ETGKRSKEGHSLEMENENLVENGADSDEDDNSFLKQQSPQEPKSLNWSSF
VDNTFAEEFTTQNQKSQDVELWEGEVVKELSVEEQIKRNRYYDEDEDEE

The term “cancer associated with a component of the NF-κB pathway downstream of a CBM complex” as used herein refers to cancers associated with or having a dysregulation of a gene, a protein, or the expression or activity or level of any (e.g., one or more) of the same associated with a component of the NF-κB pathway downstream of a CBM complex. In some embodiments, a cancer associated with a component of the NF-κB pathway downstream of a CBM complex is selected from the group consisting of a TAK1-associated cancer, a TRAF6-associated cancer, a TAB1-associated cancer, a TAB2-associated cancer, a TAB3-associated cancer, a MKK7-associated cancer, an IKKα-associated cancer, an IKKβ-associated cancer, an IKKγ-associated cancer, an IkBα-associated cancer, a p50-associated cancer, a p65 (RelA)-associated cancer, a c-Rel-associated cancer, and combinations thereof. In some embodiments, a cancer associated with a component of the NF-κB pathway downstream of a CBM complex is an IKKγ-associated cancer. The cancers “associated” with a particular gene or protein described in this paragraph refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

An exemplary sequence of human TAK1 is shown below:

SEQ ID NO: 20
(UniParc Accession No. UPI000012EAD6)
MSTASAASSSSSSSAGEMIEAPSQVLNFEEIDYKEIEVEEVVGRGAFGVV
CKAKWRAKDVAIKQIESESERKAFIVELRQLSRVNHPNIVKLYGACLNPV
CLVMEYAEGGSLYNVLHGAEPLPYYTAAHAMSWCLQCSQGVAYLHSMQPK
ALIHRDLKPPNLLLVAGGTVLKICDFGTACDIQTHMTNNKGSAAWMAPEV
FEGSNYSEKCDVFSWGIILWEVITRRKPFDEIGGPAFRIMWAVHNGTRPP
LIKNLPKPIESLMTRCWSKDPSQRPSMEEIVKIMTHLMRYFPGADEPLQY
PCQYSDEGQSNSATSTGSFMDIASTNTSNKSDTNMEQVPATNDTIKRLES
KLLKNQAKQQSESGRLSLGASRGSSVESLPPTSEGKRMSADMSEIEARIA
ATTAYSKPKRGHRKTASFGNILDVPEIVISGNGQPRRRSIQDLTVTGTEP
GQVSSRSSSPSVRMITTSGPTSEKPTRSHPWTPDDSTDTNGSDNSIPMAY
LTLDHQLQPLAPCPNSKESMAVFEQHCKMAQEYMKVQTEIALLLQRKQEL
VAELDQDEKDQQNTSRLVQEHKKLLDENKSLSTYYQQCKKQLEVIRSQQQ
KRQGTS

An exemplary sequence of human TRAF6 is shown below:

SEQ ID NO: 21
(UniParc Accession No. UPI000000D924)
MSLLNCENSCGSSQSESDCCVAMASSCSAVTKDDSVGGTASTGNLSSSFM
EEIQGYDVEFDPPLESKYECPICLMALREAVQTPCGHRFCKACIIKSIRD
AGHKCPVDNEILLENQLFPDNFAKREILSLMVKCPNEGCLHKMELRHLED
HQAHCEFALMDCPQCQRPFQKFHINIHILKDCPRRQVSCDNCAASMAFED
KEIHDQNCPLANVICEYCNTILIREQMPNHYDLDCPTAPIPCTFSTFGCH
EKMQRNHLARHLQENTQSHMRMLAQAVHSLSVIPDSGYISEVRNFQETIH
QLEGRLVRQDHQIRELTAKMETQSMYVSELKRTIRTLEDKVAEIEAQQCN
GIYIWKIGNFGMHLKCQEEEKPVVIHSPGFYTGKPGYKLCMRLHLQLPTA
QRCANYISLFVHTMQGEYDSHLPWPFQGTIRLTILDQSEAPVRQNHEEIM
DAKPELLAFQRPTIPRNPKGFGYVTFMHLEALRQRTFIKDDTLLVRCEVS
TRFDMGSLRREGFQPRSTDAGV

An exemplary sequence of human TAB1 is shown below:

SEQ ID NO: 22
(UniParc Accession No. UPI0000136861)
MAAQRRSLLQSEQQPSWTDDLPLCHLSGVGSASNRSYSADGKGTESHPPE
DSWLKFRSENNCFLYGVFNGYDGNRVTNFVAQRLSAELLLGQLNAEHAEA
DVRRVLLQAFDVVERSFLESIDDALAEKASLQSQLPEGVPQHQLPPQYQK
ILERLKTLEREISGGAMAVVAVLLNNKLYVANVGTNRALLCKSTVDGLQV
TQLNVDHTTENEDELFRLSQLGLDAGKIKQVGIICGQESTRRIGDYKVKY
GYTDIDLLSAAKSKPIIAEPEIHGAQPLDGVTGFLVLMSEGLYKALEAAH
GPGQANQEIAAMIDTEFAKQTSLDAVAQAVVDRVKRIHSDTFASGGERAR
FCPRHEDMTLLVRNFGYPLGEMSQPTPSPAPAAGGRVYPVSVPYSSAQST
SKTSVTLSLVMPSQGQMVNGAHSASTLDEATPTLTNQSPTLTLQSTNTHT
QSSSSSSDGGLERSRPAHSLPPGEDGRVEPYVDFAEFYRLWSVDHGEQSV
VTAP

An exemplary sequence of human TAB2 is shown below:

SEQ ID NO: 23
(UniParc Accession No. UPI0000073C75)
MAQGSHQIDFQVLHDLRQKFPEVPEVVVSRCMLQNNNNLDACCAVLSQES
TRYLYGEGDLNFSDDSGISGLRNHMTSLNLDLQSQNIYHHGREGSRMNGS
RTLTHSISDGQLQGGQSNSELFQQEPQTAPAQVPQGENVFGMSSSSGASN
SAPHLGFHLGSKGTSSLSQQTPRFNPIMVTLAPNIQTGRNTPTSLHIHGV
PPPVLNSPQGNSIYIRPYITTPGGTTRQTQQHSGWVSQFNPMNPQQVYQP
SQPGPWTTCPASNPLSHTSSQQPNQQGHQTSHVYMPISSPTTSQPPTIHS
SGSSQSSAHSQYNIQNISTGPRKNQIEIKLEPPQRNNSSKLRSSGPRTSS
TSSSVNSQTLNRNQPTVYIAASPPNTDELMSRSQPKVYISANAATGDEQV
MRNQPTLFISTNSGASAASRNMSGQVSMGPAFIHHHPPKSRAIGNNSATS
PRVVVTQPNTKYTFKITVSPNKPPAVSPGVVSPTFELTNLLNHPDHYVET
ENIQHLTDPTLAHVDRISETRKLSMGSDDAAYTQALLVHQKARMERLQRE
LEIQKKKLDKLKSEVNEMENNLTRRRLKRSNSISQIPSLEEMQQLRSCNR
QLQIDIDCLTKEIDLFQARGPHFNPSAIHNFYDNIGFVGPVPPKPKDQRS
IIKTPKTQDTEDDEGAQWNCTACTFLNHPALIRCEQCEMPRHF

An exemplary sequence of human TAB3 is shown below:

SEQ ID NO: 24
(UniParc Accession No. UPI0000071648)
MAQSSPQLDIQVLHDLRQRFPEIPEGVVSQCMLQNNNNLEACCRALSQES
SKYLYMEYHSPDDNRMNRNRLLHINLGIHSPSSYHPGDGAQLNGGRTLVH
SSSDGHIDPQHAAGKQLICLVQEPHSAPAVVAATPNYNPFFMNEQNRSAA
TPPSQPPQQPSSMQTGMNPSAMQGPSPPPPPPSYMHIPRYSTNPITVTVS
QNLPSGQTVPRALQILPQIPSNLYGSPGSIYIRQTSQSSSGRQTPQSTPW
QSSPQGPVPHYSQRPLPVYPHQQNYQPSQYSPKQQQIPQSAYHSPPPSQC
PSPFSSPQHQVQPSQLGHIFMPPSPSTTPPHPYQQGPPSYQKQGSHSVAY
LPYTASSLSKGSMKKIEITVEPSQRPGTAINRSPSPISNQPSPRNQHSLY
TATTPPSSSPSRGISSQPKPPFSVNPVYITYTQPTGPSCTPSPSPRVIPN
PTTVFKITVGRATTENLLNLVDQEERSAAPEPIQPISVIPGSGGEKGSHK
YQRSSSSGSDDYAYTQALLLHQRARMERLAKQLKLEKEELERLKSEVNGM
EHDLMQRRLRRVSCTTAIPTPEEMTRLRSMNRQLQINVDCTLKEVDLLQS
RGNFDPKAMNNFYDNIEPGPVVPPKPSKKDSSDPCTIERKARRISVTSKV
QADIHDTQAAAADEHRTGSTQSPRTQPRDEDYEGAPWNCDSCTFLNHPAL
NRCEQCEMPRYT

An exemplary sequence of human MKK7 is shown below:

SEQ ID NO: 25
(UniParc Accession No. UPI000012F494)
MAASSLEQKLSRLEAKLKQENREARRRIDLNLDISPQRPRPTLQLPLAND
GGSRSPSSESSPQHPTPPARPRHMLGLPSTLFTPRSMESIEIDQKLQEIM
KQTGYLTIGGQRYQAEINDLENLGEMGSGTCGQVWKMRFRKTGHVIAVKQ
MRRSGNKEENKRILMDLDVVLKSHDCPYIVQCFGTFITNTDVFIAMELMG
TCAEKLKKRMQGPIPERILGKMTVAIVKALYYLKEKHGVIHRDVKPSNIL
LDERGQIKLCDFGISGRLVDSKAKTRSAGCAAYMAPERIDPPDPTKPDYD
IRADVWSLGISLVELATGQFPYKNCKTDFEVLTKVLQEEPPLLPGHMGFS
GDFQSFVKDCLTKDHRKRPKYNKLLEHSFIKRYETLEVDVASWFKDVMAK
TESPRTSGVLSQPHLPFFR

An exemplary sequence of human IKKα is shown below:

SEQ ID NO: 26
(UniParc Accession No. UPI000013D6C7)
MERPPGLRPGAGGPWEMRERLGTGGFGNVCLYQHRELDLKIAIKSCRLEL
STKNRERWCHEIQIMKKLNHANVVKACDVPEELNILIHDVPLLAMEYCSG
GDLRKLLNKPENCCGLKESQILSLLSDIGSGIRYLHENKIIHRDLKPENI
VLQDVGGKIIHKIIDLGYAKDVDQGSLCTSFVGTLQYLAPELFENKPYTA
TVDYWSFGTMVFECIAGYRPFLHHLQPFTWHEKIKKKDPKCIFACEEMSG
EVRFSSHLPQPNSLCSLVVEPMENWLQLMLNWDPQQRGGPVDLTLKQPRC
FVLMDHILNLKIVHILNMTSAKIISFLLPPDESLHSLQSRIERETGINTG
SQELLSETGISLDPRKPASQCVLDGVRGCDSYMVYLFDKSKTVYEGPFAS
RSLSDCVNYIVQDSKIQLPIIQLRKVWAEAVHYVSGLKEDYSRLFQGQRA
AMLSLLRYNANLTKMKNTLISASQQLKAKLEFFHKSIQLDLERYSEQMTY
GISSEKMLKAWKEMEEKAIHYAEVGVIGYLEDQIMSLHAEIMELQKSPYG
RRQGDLMESLEQRAIDLYKQLKHRPSDHSYSDSTEMVKIIVHTVQSQDRV
LKELFGHLSKLLGCKQKIIDLLPKVEVALSNIKEADNTVMFMQGKRQKEI
WHLLKIACTQSSARSLVGSSLEGAVTPQTSAWLPPTSAEHDHSLSCVVTP
QDGETSAQMIEENLNCLGHLSTIIHEANEEQGNSMMNLDWSWLTE

An exemplary sequence of human IKKβ is shown below:

SEQ ID NO: 27
(UniParc Accession No. UPI0000033729)
MSWSPSLTTQTCGAWEMKERLGTGGFGNVIRWHNQETGEQIAIKQCRQEL
SPRNRERWCLEIQIMRRLTHPNVVAARDVPEGMQNLAPNDLPLLAMEYCQ
GGDLRKYLNQFENCCGLREGAILTLLSDIASALRYLHENRIIHRDLKPEN
IVLQQGEQRLIHKIIDLGYAKELDQGSLCTSFVGTLQYLAPELLEQQKYT
VTVDYWSFGTLAFECITGFRPFLPNWQPVQWHSKVRQKSEVDIVVSEDLN
GTVKFSSSLPYPNNLNSVLAERLEKWLQLMLMWHPRQRGTDPTYGPNGCF
KALDDILNLKLVHILNMVTGTIHTYPVTEDESLQSLKARIQQDTGIPEED
QELLQEAGLALIPDKPATQCISDGKLNEGHTLDMDLVFLFDNSKITYETQ
ISPRPQPESVSCILQEPKRNLAFFQLRKVWGQVWHSIQTLKEDCNRLQQG
QRAAMMNLLRNNSCLSKMKNSMASMSQQLKAKLDFFKTSIQIDLEKYSEQ
TEFGITSDKLLLAWREMEQAVELCGRENEVKLLVERMMALQTDIVDLQRS
PMGRKQGGTLDDLEEQARELYRRLREKPRDQRTEGDSQEMVRLLLQAIQS
FEKKVRVIYTQLSKTVVCKQKALELLPKVEEVVSLMNEDEKTVVRLQEKR
QKELWNLLKIACSKVRGPVSGSPDSMNASRLSQPGQLMSQPSTASNSLPE
PAKKSEELVAEAHNLCTLLENAIQDTVREQDQSFTALDWSWLQTEEEEHS
CLEQAS

An exemplary sequence of human IKKγ is shown below:

SEQ ID NO: 28
(UniParc Accession No. UPI0000000CC4)
MNRHLWKSQLCEMVQPSGGPAADQDVLGEESPLGKPAMLHLPSEQGAPET
LQRCLEENQELRDAIRQSNQILRERCEELLHFQASQREEKEFLMCKFQEA
RKLVERLGLEKLDLKRQKEQALREVEHLKRCQQQMAEDKASVKAQVTSLL
GELQESQSRLEAATKECQALEGRARAASEQARQLESEREALQQQHSVQVD
QLRMQGQSVEAALRMERQAASEEKRKLAQLQVAYHQLFQEYDNHIKSSVV
GSERKRGMQLEDLKQQLQQAEEALVAKQEVIDKLKEEAEQHKIVMETVPV
LKAQADIYKADFQAERQAREKLAEKKELLQEQLEQLQREYSKLKASCQES
ARIEDMRKRHVEVSQAPLPPAPAYLSSPLALPSQRRSPPEEPPDFCCPKC
QYQAPDMDTLQIHVMECIE

Non-limiting examples of dysregulation of an IKKγ gene or an IKKγ protein are described in, for example, Courtois and Gilmore, Oncogene 25.51 (2006): 6831-6843.

An exemplary sequence of human IkBα is shown below:

SEQ ID NO: 29
(UniParc Accession No. UPI000004F0A9)
MFQAAERPQEWAMEGPRDGLKKERLLDDRHDSGLDSMKDEEYEQMVKELQ
EIRLEPQEVPRGSEPWKQQLTEDGDSFLHLAIIHEEKALTMEVIRQVKGD
LAFLNFQNNLQQTPLHLAVITNQPEIAEALLGAGCDPELRDFRGNTPLHL
ACEQGCLASVGVLTQSCTTPHLHSILKATNYNGHTCLHLASIHGYLGIVE
LLVSLGADVNAQEPCNGRTALHLAVDLQNPDLVSLLLKCGADVNRVTYQG
YSPYQLTWGRPSTRIQQQLGQLTLENLQMLPESEDEESYDTESEFTEFTE
DELPYDDCVFGGQRLTL

An exemplary sequence of human p105, which is processed into p50, is shown below:

SEQ ID NO: 30
(UniParc Accession No. UPI000000D917)
MAEDDPYLGRPEQMFHLDPSLTHTIFNPEVFQPQMALPTDGPYLQILEQP
KQRGFRFRYVCEGPSHGGLPGASSEKNKKSYPQVKICNYVGPAKVIVQLV
INGKNIHLHAHSLVGKHCEDGICTVTAGPKDMVVGFANLGILHVTKKKVF
ETLEARMTEACIRGYNPGLLVHPDLAYLQAEGGGDRQLGDREKELIRQAA
LQQTKEMDLSVVRLMFTAFLPDSTGSFTRRLEPVVSDAIYDSKAPNASNL
KIVRMDRTAGCVTGGEEIYLLCDKVQKDDIQIRFYEEEENGGVWEGFGDF
SPTDVHRQFAIVFKTPKYKDINITKPASVFVQLRRKSDLETSEPKPFLYY
PEIKDKEEVQRKRQKLMPNFSDSFGGGSGAGAGGGGMFGSGGGGGGTGST
GPGYSFPHYGFPTYGGITFHPGTTKSNAGMKHGTMDTESKKDPEGCDKSD
DKNTVNLFGKVIETTEQDQEPSEATVGNGEVTLTYATGTKEESAGVQDNL
FLEKAMQLAKRHANALFDYAVTGDVKMLLAVQRHLTAVQDENGDSVLHLA
IIHLHSQLVRDLLEVTSGLISDDIINMRNDLYQTPLHLAVITKQEDVVED
LLRAGADLSLLDRLGNSVLHLAAKEGHDKVLSILLKHKKAALLLDHPNGD
GLNAIHLAMMSNSLPCLLLLVAAGADVNAQEQKSGRTALHLAVEHDNISL
AGCLLLEGDAHVDSTTYDGTTPLHIAAGRGSTRLAALLKAAGADPLVENF
EPLYDLDDSWENAGEDEGVVPGTTPLDMATSWQVFDILNGKPYEPEFTSD
DLLAQGDMKQLAEDVKLQLYKLLEIPDPDKNWATLAQKLGLGILNNAFRL
SPAPSKTLMDNYEVSGGTVRELVEALRQMGYTEAIEVIQAASSPVKTTSQ
AHSLPLSPASTRQQIDELRDSDSVCDSGVETSFRKLSFTESLTSGASLLT
LNKMPHDYGQEGPLEGKI

An exemplary sequence of human p65 is shown below:

SEQ ID NO: 31
(UniParc Accession No. UPI000013ED68)
MDELFPLIFPAEPAQASGPYVEIIEQPKQRGMRFRYKCEGRSAGSIPGER
STDTTKTHPTIKINGYTGPGTVRISLVTKDPPHRPHPHELVGKDCRDGFY
EAELCPDRCIHSFQNLGIQCVKKRDLEQAISQRIQTNNNPFQVPIEEQRG
DYDLNAVRLCFQVTVRDPSGRPLRLPPVLSHPIFDNRAPNTAELKICRVN
RNSGSCLGGDEIFLLCDKVQKEDIEVYFTGPGWEARGSFSQADVHRQVAI
VFRTPPYADPSLQAPVRVSMQLRRPSDRELSEPMEFQYLPDTDDRHRIEE
KRKRTYETFKSIMKKSPFSGPTDPRPPPRRIAVPSRSSASVPKPAPQPYP
FTSSLSTINYDEFPTMVFPSGQISQASALAPAPPQVLPQAPAPAPAPAMV
SALAQAPAPVPVLAPGPPQAVAPPAPKPTQAGEGTLSEALLQLQFDDEDL
GALLGNSTDPAVFTDLASVDNSEFQQLLNQGIPVAPHTTEPMLMEYPEAI
TRLVTGAQRPPDPAPAPLGAPGLPNGLLSGDEDFSSIADMDESALLSQIS
S

An exemplary sequence of human c-Rel is shown below:

SEQ ID NO: 32
(UniParc Accession No. UPI000013367B)
MASGAYNPYIEIIEQPRQRGMRFRYKCEGRSAGSIPGEHSTDNNRTYPSI
QIMNYYGKGKVRITLVTKNDPYKPHPHDLVGKDCRDGYYEAEFGQERRPL
FFQNLGIRCVKKKEVKEAIITRIKAGINPFNVPEKQLNDIEDCDLNVVRL
CFQVFLPDEHGNLTTALPPVVSNPIYDNRAPNTAELRICRVNKNCGSVRG
GDEIFLLCDKVQKDDIEVRFVLNDWEAKGIFSQADVHRQVAIVFKTPPYC
KAITEPVTVKMQLRRPSDQEVSESMDFRYLPDEKDTYGNKAKKQKTTLLF
QKLCQDHVETGFRHVDQDGLELLTSGDPPTLASQSAGITVNFPERPRPGL
LGSIGEGRYFKKEPNLFSHDAVVREMPTGVSSQAESYYPSPGPISSGLSH
HASMAPLPSSSWSSVAHPTPRSGNTNPLSSFSTRTLPSNSQGIPPFLRIP
VGNDLNASNACIYNNADDIVGMEASSMPSADLYGISDPNMLSNCSVNMMT
TSSDSMGETDNPRLLSMNLENPSCNSVLDPRDLRQLHQMSSSSMSAGANS
NTTVFVSQSDAFEGSDFSCADNSMINESGPSNSTNPNSHGFVQDSQYSGI
GSMQNEQLSDSFPYEFFQV

The term “cancer associated with a component of the JNK pathway downstream of a CBM complex” as used herein refers to cancers associated with or having a dysregulation of a gene, a protein, or the expression or activity or level of any (e.g., one or more) of the same associated with a component of the JNK pathway downstream of a CBM complex. In some embodiments, a cancer associated with a component of the JNK pathway downstream of a CBM complex is selected from the group consisting of a JNK1-associated cancer, a JNK2-associated cancer, a JNK3-associated cancer, a MYD88 transcription factor-associated cancer, an AP-1 transcription factor-associated cancer, and combinations thereof. The cancers “associated” with a particular gene or protein described in this paragraph refer to cancers associated with or having a dysregulation of the particular gene, the particular protein, or the expression or activity or level of any (e.g., one or more) of the same (e.g., any of the types of dysregulation of the particular gene, the particular protein, or the expression or activity or level of any of the same described herein). Non-limiting examples of such cancers are described herein.

An exemplary sequence of human JNK1 is shown below:

SEQ ID NO: 33
(UniParc Accession No. UPI000012F17A)
MSRSKRDNNFYSVEIGDSTFTVLKRYQNLKPIGSGAQGIVCAAYDAILER
NVAIKKLSRPFQNQTHAKRAYRELVLMKCVNHKNIIGLLNVFTPQKSLEE
FQDVYIVMELMDANLCQVIQMELDHERMSYLLYQMLCGIKHLHSAGIIHR
DLKPSNIVVKSDCTLKILDFGLARTAGTSFMMTPYVVTRYYRAPEVILGM
GYKENVDLWSVGCIMGEMVCHKILFPGRDYIDQWNKVIEQLGTPCPEFMK
KLQPTVRTYVENRPKYAGYSFEKLFPDVLFPADSEHNKLKASQARDLLSK
MLVIDASKRISVDEALQHPYINVWYDPSEAEAPPPKIPDKQLDEREHTIE
EWKELIYKEVMDLEERTKNGVIRGQPSPLGAAVINGSQHPSSSSSVNDVS
SMSTDPTLASDTDSSLEAAAGPLGCCR

An exemplary sequence of human JNK2 is shown below:

SEQ ID NO: 34
(UniParc Accession No. UPI000006E3AD)
MSDSKCDSQFYSVQVADSTFTVLKRYQQLKPIGSGAQGIVCAAFDTVLGI
NVAVKKLSRPFQNQTHAKRAYRELVLLKCVNHKNIISLLNVFTPQKTLEE
FQDVYLVMELMDANLCQVIHMELDHERMSYLLYQMLCGIKHLHSAGIIHR
DLKPSNIVVKSDCTLKILDFGLARTACTNFMMTPYVVTRYYRAPEVILGM
GYKENVDIWSVGCIMGELVKGCVIFQGTDHIDQWNKVIEQLGTPSAEFMK
KLQPTVRNYVENRPKYPGIKFEELFPDWIFPSESERDKIKTSQARDLLSK
MLVIDPDKRISVDEALRHPYITVWYDPAEAEAPPPQIYDAQLEEREHAIE
EWKELIYKEVMDWEERSKNGVVKDQPSDAAVSSNATPSQSSSINDISSMS
TEQTLASDTDSSLDASTGPLEGCR

An exemplary sequence of human JNK3 is shown below:

SEQ ID NO: 35
(UniParc Accession No. UPI0000049042)
MSLHFLYYCSEPTLDVKIAFCQGFDKQVDVSYIAKHYNMSKSKVDNQFYS
VEVGDSTFTVLKRYQNLKPIGSGAQGIVCAAYDAVLDRNVAIKKLSRPFQ
NQTHAKRAYRELVLMKCVNHKNIISLLNVFTPQKTLEEFQDVYLVMELMD
ANLCQVIQMELDHERMSYLLYQMLCGIKHLHSAGIIHRDLKPSNIVVKSD
CTLKILDFGLARTAGTSFMMTPYVVTRYYRAPEVILGMGYKENVDIWSVG
CIMGEMVRHKILFPGRDYIDQWNKVIEQLGTPCPEFMKKLQPTVRNYVEN
RPKYAGLTFPKLFPDSLFPADSEHNKLKASQARDLLSKMLVIDPAKRISV
DDALQHPYINVWYDPAEVEAPPPQIYDKQLDEREHTIEEWKELIYKEVMN
SEEKTKNGVVKGQPSPSGAAVNSSESLPPSSSVNDISSMSTDQTLASDTD
SSLEASAGPLGCCR

### Compounds of Formula (I)

Provided herein are compounds of Formula (I), or a pharmaceutically acceptable salt thereof:

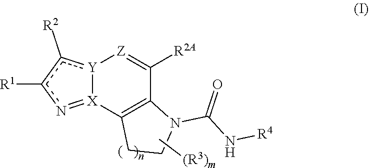

wherein:
each [symbol] is a single or double bond;
X is N or C;
Y is N or C;
Z is N or CR⁵;
wherein when one of X and Y is N, the other of X and Y is C;
n is 1, 2, or 3;
R¹ is hydrogen, halogen, cyano, hydroxyl, C1-C3 alkoxy, C1-C3 haloalkoxy, C1-C3 haloalkyl, —NR^{A}R^{B}, or C1-C3 alkyl optionally substituted with 1-3 substituents independently selected from hydroxyl and C1-C3 alkoxy;
R² is hydrogen, amino, or halogen;
R^{2A} is hydrogen, halogen, or C1-C6 alkyl;
each R³ is independently halogen, hydroxyl, cyano, C3-C6 cycloalkyl, —NR^{A}R^{B}, 5-6 membered heteroaryl optionally substituted with C1-C3 alkyl; C1-C3 alkoxy, C1-C3 haloalkoxy, C1-C3 haloalkyl, or C1-C3 alkyl optionally substituted with a C1-C3 alkoxy or cyano; or two R³ together with the carbon atom to which they are attached come together to form an oxo group or a C3-C8 cycloalkyl;
m is 0, 1, 2, or 3;
R is phenyl, naphthyl, 5-10 membered heteroaryl, 3-10 membered heterocyclyl, or C3-C8 cycloalkyl; wherein each R group is optionally substituted with 1-3 substituents independently selected from R⁶;
R⁵ is hydrogen, halogen, cyano, hydroxyl, C1-C3 alkoxy, C1-C3 haloalkoxy, C1-C3 haloalkyl, —NR^{C}R^{D}, or C1-C3 alkyl; and
each R⁶ is independently selected from halogen; cyano; hydroxyl; —CO₂H; —N═(S═O)(C1-C3 alkyl)₂, —S(═O)ₚ(C1-C3 alkyl), —NR^{E}R^{F}; —(C═O)NR^{E}R^{F}; C1-C3 alkoxy optionally substituted with amino, hydroxyl, or —(C═O)NR^{E}R^{F}; C1-C3 haloalkyl; C1-C3 haloalkoxy; 5-6 membered heteroaryl optionally substituted with 1-3 independently selected R^{X}; C1-C3 alkyl optionally substituted with 1-2 substituents independently selected from hydroxyl, —NR^{E}R^{F}, C1-C3 alkoxy, and C3-C6 cycloalkyl; C3-C6 cycloalkyl optionally substituted with hydroxyl; and -(Q)_{q}-3-8 membered heterocyclyl optionally substituted with 1-3 independently selected C1-C3 alkyl;
p is 1 or 2;
Q is —O— or —NH—;
q is 0 or 1;
each R^{X} is independently selected from halogen, cyano, hydroxyl, amino, C1-C3 alkoxy, C1-C3 haloalkoxy, C1-C3 haloalkyl, or C1-C6 alkyl optionally substituted with 1-3 substituents independently selected from hydroxyl, C1-C3 alkoxy, and —NR^{G}R^{H}; and
R^{A}, R^{B}, R^{C}, R^{D} are independently hydrogen, C1-C3 alkyl, or R^{A} and R^{B}, or R^{C} and R^{D}, together with the nitrogen atom to which they are attached come together to form a 4-6 membered heterocyclyl; and
R^{E}, R^{F}, R^{G}, and R^{H} are independently hydrogen, C1-C3 alkyl, or C3-C6 cycloalkyl, or R^{E} and R^{F}, or R^{G} and R^{H}, together with the nitrogen atom to which they are attached come together to form a 4-6 membered heterocyclyl optionally substituted with C1-C3 alkyl or C1-C3 alkoxy.

In some embodiments:
each [symbol] is a single or double bond;
X is N or C;
Y is N or C;
Z is N or CR⁵;
wherein when one of X and Y is N, the other of X and Y is C;
n is 1, 2, or 3;
R¹ is hydrogen, halogen, cyano, hydroxyl, C1-C3 alkoxy, C1-C3 haloalkoxy, C1-C3 haloalkyl, —NR^{A}R^{B}, or C1-C3 alkyl optionally substituted with 1-3 substituents independently selected from hydroxyl and C1-C3 alkoxy;
R² is hydrogen or halogen;
R^{2A} is hydrogen;
each R³ is independently halogen, hydroxyl, C3-C6 cycloalkyl, C1-C3 alkoxy, C1-C3 haloalkoxy, C1-C3 haloalkyl, or C1-C3 alkyl optionally substituted with a C1-C3 alkoxy; or two R³ together with the carbon atom to which they are attached come together to form an oxo group or a C3-C8 cycloalkyl;
m is 0, 1, 2, or 3;
R⁴ is phenyl, 5-6 membered heteroaryl, 3-10 membered heterocyclyl, or C3-C8 cycloalkyl; wherein each R⁴ group is optionally substituted with 1-3 substituents independently selected from R⁶;
R⁵ is hydrogen, halogen, cyano, hydroxyl, C1-C3 alkoxy, C1-C3 haloalkoxy, C1-C3 haloalkyl, —NR^{C}R^{D}, or C1-C3 alkyl; and
each R⁶ is independently selected from halogen; cyano; —CO₂H; —NR^{E}R^{F}; —(C═O)NR^{E}R^{F}; C1-C3 alkoxy; C1-C3 haloalkyl; C1-C3 haloalkoxy; 5-6 membered heteroaryl optionally substituted with C1-C3 alkyl optionally substituted with hydroxyl, —NR^{E}R^{F}, or C1-C3 alkoxy; and 3-8 membered heterocyclyl;
R^{A}, R^{B}, R^{C}, R^{D} are independently hydrogen, C1-C3 alkyl, or R^{A} and R^{B}, or R^{C} and R^{D}, together with the nitrogen atom to which they are attached come together to form a 4-6 membered heterocyclyl; and
R^{E} and R^{F} are independently hydrogen, C1-C3 alkyl, or R^{E} and R^{F}, together with the nitrogen atom to which they are attached come together to form a 4-6 membered heterocyclyl optionally substituted with C1-C3 alkyl or C1-C3 alkoxy.

In some embodiments, the five-membered nitrogen-containing ring, formed in part by X and Y, is a heteroaromatic ring.

In some embodiments, X is C and Y is C.

In some embodiments, X is N and Y is C.

In some embodiments, X is C and Y is N.

In some embodiments, Z is N. In some embodiments, Z is CR⁵.

In some embodiments, X is C; Y is C; and Z is CR⁵. In some embodiments, X is N; Y is C; and Z is CR⁵. In some embodiments, X is C; Y is N; and Z is CR⁵. In some embodiments, X is C; Y is C; and Z is N. In some embodiments, X is N; Y is C; and Z is N. In some embodiments, X is C; Y is N; and Z is N.

In some embodiments, R¹ is hydrogen.

In some embodiments, R¹ is halogen, cyano, hydroxyl, C1-C3 alkoxy, C1-C3 haloalkyl, —NR^{A}R^{B}, or C1-C3 alkyl optionally substituted with 1-3 substituents independently selected from hydroxyl and C1-C3 alkoxy.

In some embodiments, R¹ is halogen or cyano. In some embodiments, R¹ is chloro or cyano. In some embodiments, R¹ is halogen. For example, R¹ is fluoro. For example, R¹ is chloro. In some embodiments, R¹ is cyano. In some embodiments, R¹ is hydroxyl.

In some embodiments, R¹ is C1-C3 alkoxy. In some embodiments, R¹ is methoxy or ethoxy.

In some embodiments, R¹ is C1-C3 haloalkoxy. In some embodiments, R¹ is trifluoromethoxy, difluoromethoxy, or fluoromethoxy.

In some embodiments, R¹ is C1-C3 haloalkyl. In some embodiments, R¹ is trifluoromethyl or 2,2,2-trifluoroethyl.

In some embodiments, R¹ is —NR^{A}R^{B}. In some embodiments, R^{A} and R^{B} are independently hydrogen or C1-C3 alkyl. In certain embodiments, one of R^{A} and R^{B} is hydrogen and the other of R^{A} and R^{B} is C1-C3 alkyl. In some embodiments, one of R^{A} and R^{B} is hydrogen and the other of R^{A} and R^{B} is methyl. In some embodiments, one of R^{A} and R^{B} is hydrogen and the other of R^{A} and R^{B} is ethyl. In certain embodiments, R^{A} and R^{B} are both hydrogens. In certain embodiments, R^{A} and R^{B} are both C1-C3 alkyl. In some embodiments, R^{A} and R^{B} are both methyl. In some embodiments, one of R^{A} and R^{B} is methyl and the other of R^{A} and R^{B} is ethyl. In some embodiments, R^{A} and R^{B} are both ethyl.

In some embodiments, R^{A} and R^{B} together with the nitrogen atom to which they are attached come together to form a 4-6 membered heterocyclyl. In certain embodiments, R^{A} and R^{B} together with the nitrogen atom to which they are attached come together to form a 4 membered heterocyclyl. In some embodiments, R^{A} and R^{B} together with the nitrogen atom to which they are attached come together to form a 5 membered heterocyclyl. In some embodiments, R^{A} and R^{B} together with the nitrogen atom to which they are attached come together to form a 6 membered heterocyclyl.

In some embodiments, R¹ is C1-C3 alkyl optionally substituted with 1-3 substituents independently selected from hydroxyl and C1-C3 alkoxy. In certain embodiments, R¹ is C1-C3 alkyl optionally substituted with 1 substituent selected from hydroxyl and C1-C3 alkoxy. In certain of these embodiments, R¹ is methyl optionally substituted with 1 substituent selected from hydroxyl and C1-C3 alkoxy. In certain embodiments, R¹ is ethyl optionally substituted with 1 substituent selected from hydroxyl and C1-C3 alkoxy. In certain embodiments, R is C1-C3 alkyl optionally substituted with hydroxyl. In certain embodiments, R¹ is C1-C3 alkyl optionally substituted with C1-C3 alkoxy (e.g., methoxy). In some embodiments, R¹ is hydroxymethyl or methoxyethyl.

In some embodiments, R¹ is unsubstituted C1-C3 alkyl (e.g., methyl or ethyl).

In some embodiments, R² is hydrogen. In some embodiments, R² is halogen. For example, R² is fluoro. For example, R² is chloro. In some embodiments, R² is amino.

In some embodiments, R^{2A} is hydrogen. In some embodiments, R^{2A} is halogen, for example, R^{2A} is fluoro or chloro. In some embodiments, R^{2A} is C1-C6 alkyl, such as those described herein.

In some embodiments, n is 1, 2, or 3. In some embodiments, n is 1 or 2. In some embodiments, n is 2 or 3. In some embodiments, n is 1 or 3. In some embodiments, n is 1. In some embodiments, n is 2. In some embodiments, n is 3.

In some embodiments, m is 0, 1, 2, or 3. In some embodiments, m is 0, 1, or 2. In some embodiments, m is 1, 2, or 3. In some embodiments, m is 0, 2, or 3. In some embodiments, m is 0, 1, or 3. In some embodiments, m is 0 or 1. In some embodiments, m is 0 or 2. In some embodiments, m is 0 or 3. In some embodiments, m is 1 or 2. In some embodiments, m is 1 or 3. In some embodiments, m is 2 or 3. In some embodiments, m is 0. In some embodiments, m is 1. In some embodiments, m is 2. In some embodiments, m is 3.

In some embodiments, each R³ is independently halogen, cyano, C3-C6 cycloalkyl, C1-C3 alkyl optionally substituted with C1-C3 alkoxy or cyano, C1-C3 haloalkyl, C1-C3 alkoxy, or C1-C3 haloalkoxy. In some embodiments, each R³ is independently C3-C6 cycloalkyl, C1-C3 alkyl optionally substituted with C1-C3 alkoxy or cyano, C1-C3 haloalkyl, C1-C3 alkoxy, or C1-C3 haloalkoxy. In some embodiments, each R³ is independently unsubstituted C1-C3 alkyl or C1-C3 haloalkyl. In some embodiments, each R³ is independently cyclopropyl, methyl optionally substituted with methoxy, trifluoromethyl, methoxy, or trifluoromethoxy. In some embodiments, each R³ is independently cyclopropyl, methyl, methoxymethyl, or trifluoromethyl. In some embodiments, each R³ is independently hydroxyl, C3-C6 cycloalkyl, C1-C3 alkyl optionally substituted with C1-C3 alkoxy, or C1-C3 haloalkyl. In some embodiments, each R³ is independently hydroxyl, cyclopropyl, methyl optionally substituted with methoxy, or trifluoromethyl.

In some embodiments, each R³ is independently hydroxyl, cyano, —NR^{A}R^{B}, 5-6 membered heteroaryl optionally substituted with C1-C3 alkyl; C3-C6 cycloalkyl, C1-C3 alkyl optionally substituted with a C1-C3 alkoxy or a cyano, or C1-C3 haloalkyl. In some embodiments, each R³ is independently hydroxyl, C3-C6 cycloalkyl, C1-C3 alkyl substituted with a C1-C3 alkoxy, or C1-C3 haloalkyl. In some embodiments, each R³ is independently hydroxyl, cyano, C3-C6 cycloalkyl, C1-C3 alkyl, or C1-C3 haloalkyl.

In some embodiments, each R³ is independently halogen. For example, an R³ is fluoro or chloro. In some embodiments, each R³ is independently hydroxyl.

In some embodiments, each R³ is independently C3-C6 cycloalkyl, C1-C3 alkyl, C1-C3 alkoxy, C1-C3 haloalkoxy, or C1-C3 haloalkyl.

In some embodiments, each R³ is independently hydroxyl.

In some embodiments, each R³ is independently cyano.

In some embodiments, each R³ is independently C3-C6 cycloalkyl. In some embodiments, each R³ is independently C3-C5 cycloalkyl. In some embodiments, each R³ is independently cyclopropyl. In some embodiments, each R³ is independently C3-C6 cycloalkyl and m is 1 or 2. In some embodiments, when m is 2, one R³ is C3-C6 cycloalkyl and the other R³ is not C3-C6 cycloalkyl.

In some embodiments, each R³ is independently —NR^{A}R^{B}. In some embodiments, each R³ is independently —NR^{A}R^{B} and m is 1 or 2. In some embodiments when m is 2, one R³ is —NR^{A}R^{B} and the other R³ is not —NR^{A}R^{B}.

In some embodiments, each R³ is independently 5-6 membered heteroaryl optionally substituted with C1-C3 alkyl. In some embodiments, each R³ is independently 5-6 membered heteroaryl optionally substituted with C1-C3 alkyl and m is 1 or 2. In some embodiments when m is 2, one R³ is 5-6 membered heteroaryl optionally substituted with C1-C3 alkyl and the other R³ is not 5-6 membered heteroaryl. In some embodiments, each R³ is independently 5-6 membered heteroaryl substituted with C1-C3 alkyl. In some embodiments, each R³ is independently 5-6 membered heteroaryl substituted with C1-C3 alkyl and m is 1 or 2. In some embodiments when m is 2, one R³ is 5-6 membered heteroaryl substituted with C1-C3 alkyl and the other R³ is not 5-6 membered heteroaryl. In some embodiments, each R³ is independently 5-6 membered heteroaryl. In some embodiments, each R³ is independently 5-6 membered heteroaryl and m is 1 or 2. In some embodiments when m is 2, one R³ is 5-6 membered heteroaryl and the other R³ is not 5-6 membered heteroaryl.

In some embodiments, each R³ is independently C1-C3 alkyl optionally substituted with a C1-C3 alkoxy or a cyano. In some embodiments, each R³ is independently C1-C3 alkyl. For example, an R³ is methyl or ethyl. In some embodiments, each R³ is independently C1-C3 alkyl substituted with a C1-C3 alkoxy, such as methoxy, ethoxy, n-propoxy, or isopropoxy. In some embodiments, an R³ is methoxymethyl or methoxyethyl. In some embodiments, each R³ is independently C1-C3 alkyl substituted with a cyano, such as cyanomethyl, or 1- or 2-cyanoethyl. In some embodiments, each R³ is independently C1-C3 alkoxy. For example, R³ is methoxy or ethoxy. In some embodiments, each R³ is independently C1-C3 haloalkoxy. For example, an R³ is trifluoromethoxy, difluoromethoxy, or fluoromethoxy. In some embodiments, each R³ is independently C1-C3 haloalkyl. For example, each R³ is trifluoromethyl or 2,2,2-trifluoroethyl.

In some embodiments, m is 1 and R³ is C1-C3 alkyl optionally substituted with C1-C3 alkoxy or cyano. In some embodiments, m is 2 and each R³ is independently C1-C3 alkyl optionally substituted with C1-C3 alkoxy or cyano. In some embodiments, m is 1 and R³ is C1-C3 alkyl. In some embodiments, m is 2 and each R³ is independently C1-C3 alkyl. In some embodiments, m is 1 and R³ is C1-C3 alkyl substituted with C1-C3 alkoxy. In some embodiments, m is 2 and each R³ is independently C1-C3 alkyl substituted with C1-C3 alkoxy. In some embodiments, m is 1 and R³ is C1-C3 alkyl substituted with cyano. In some embodiments, m is 2 and each R³ is independently C1-C3 alkyl substituted with cyano.

In some embodiments, m is 2 and each R³ is independently C1-C3 alkyl optionally substituted with C1-C3 alkoxy, the R³ groups are geminal C1-C3 alkyl groups, each optionally substituted with C1-C3 alkoxy. In some embodiments, each R³ is independently C1-C3 alkyl optionally substituted with C1-C3 alkoxy. In some embodiments, one R³ group is methyl or methoxymethyl.

In some embodiments, each R³ is independently C1-C3 alkoxy. In some embodiments, each R³ is independently C1-C3 alkoxy and m is 1 or 2. In some embodiments when m is 2, one R³ is C1-C3 alkoxy and the other R³ is not C1-C3 alkoxy. In certain of these embodiments, the C1-C3 alkoxy is methoxy.

In some embodiments, each R³ is independently C1-C3 haloalkoxy. In some embodiments, each R³ is independently C1-C3 haloalkoxy and m is 1 or 2. In some embodiments when m is 2, one R³ is C1-C3 haloalkoxy and the other R³ is not C1-C3 haloalkoxy. In certain of these embodiments, the C1-C3 haloalkoxy is trifluoromethoxy.

In some embodiments, each R³ is independently C1-C3 haloalkyl. In some embodiments, each R³ is independently C1-C3 haloalkyl and m is 1 or 2. In some embodiments when m is 2, one R³ is C1-C3 haloalkyl and the other R³ is not C1-C3 haloalkyl. In certain of these embodiments, the C1-C3 haloalkyl is trifluoromethyl.

In some embodiments, m is 2, and the R³ groups are geminal. In some embodiments, m is 2, and each R³ is independently C1-C3 haloalkyl. In some embodiments, the R³ groups are geminal independently selected C1-C3 haloalkyl groups In some embodiments, m is 2, one R³ is C1-C3 alkyl optionally substituted with C1-C3 alkoxy or cyano, and the other R³ is C1-C3 haloalkyl. In some embodiments, m is 2, one R³ is C1-C3 alkyl substituted with C1-C3 alkoxy or cyano, and the other R³ is C1-C3 haloalkyl. In some embodiments, m is 2, one R³ is C1-C3 alkyl and the other R³ is C1-C3 haloalkyl. In some embodiments, the R³ groups are geminal C1-C3 alkyl (optionally substituted with C1-C3 alkoxy or cyano) and C1-C3 haloalkyl groups In some embodiments, the R³ groups are geminal C1-C3 alkyl (substituted with C1-C3 alkoxy or cyano) and C1-C3 haloalkyl groups. In some embodiments, the R³ groups are geminal C1-C3 alkyl and C1-C3 haloalkyl groups. In some embodiments, m is 2, one R³ is C1-C3 alkyl optionally substituted with C1-C3 alkoxy or cyano, and the other R³ is C3-C6 cycloalkyl. In some embodiments, m is 2, one R³ is C1-C3 alkyl substituted with C1-C3 alkoxy and the other R³ is C3-C6 cycloalkyl. In some embodiments, m is 2, one R³ is C1-C3 alkyl substituted with cyano and the other R³ is C3-C6 cycloalkyl. In some embodiments, m is 2, one R³ is C1-C3 alkyl and the other R³ is C3-C6 cycloalkyl. In some embodiments, the R³ groups are geminal C1-C3 alkyl (optionally substituted with C1-C3 alkoxy or cyano) and C3-C6 cycloalkyl groups. In some embodiments, the R³ groups are geminal C1-C3 alkyl (substituted with C1-C3 alkoxy or cyano) and C3-C6 cycloalkyl groups. In some embodiments, the R³ groups are geminal C1-C3 alkyl and C3-C6 cycloalkyl groups. In some embodiments, m is 2, one R³ is C1-C3 haloalkyl and the other R³ is C3-C6 cycloalkyl. In some embodiments, the R³ groups are geminal C1-C3 haloalkyl and C3-C6 cycloalkyl groups.

In some embodiments, m is 1 and R³ is methyl, methoxymethyl, trifluoromethyl, or cyclopropyl. In some embodiments, m is 2 and each R³ is methyl. In some embodiments, m is 2 and each R³ is trifluoromethyl. In some embodiments, m is 2 and one R³ is methyl and the other R³ is methoxy. In some embodiments, m is 2 and one R³ is cyclopropyl and the other R³ is methoxy.

In some embodiments, m is 1 and each R³ is methyl. In some embodiments, m is 2 and each R³ is methyl. In some embodiments, m is 2, each R³ is methyl, and the R³ groups are geminal methyl groups. In some embodiments, each R³ is methyl. In some embodiments, m is 1 and R³ is methoxymethyl. In some embodiments, m is 2 and one R³ is methyl. In some embodiments, m is 2 and one R³ is methoxymethyl. In some embodiments, m is 2, each R³ is methyl, and the R³ groups are geminal methyl groups. In some embodiments, m is 2 and the R³ groups are germinal methyl and methoxymethyl groups.

In some embodiments, m is 2, and the R³ groups are geminal. In some embodiments, m is 2, and each R³ is trifluoromethyl. In some embodiments, the R³ groups are geminal trifluoromethyl groups. In some embodiments, m is 2, one R³ is C1-C3 alkyl, optionally substituted with C1-C3 alkoxy or cyano, and the other R³ is trifluoromethyl. In some embodiments, m is 2, one R³ is C1-C3 alkyl substituted with C1-C3 alkoxy, and the other R³ is trifluoromethyl. In some embodiments, m is 2, one R³ is C1-C3 alkyl and the other R³ is trifluoromethyl. In some embodiments, m is 2, one R³ is methyl and the other R³ is trifluoromethyl. In some embodiments, m is 2, one R³ is methoxymethyl and the other R³ is trifluoromethyl. In some embodiments, the R³ groups are geminal methyl and trifluoromethyl groups. In some embodiments, the R³ groups are geminal methoxymethyl and trifluoromethyl groups. In some embodiments, m is 2, one R³ is methyl and the other R³ is cyclopropyl. In some embodiments, m is 2, one R³ is methoxymethyl and the other R³ is cyclopropyl. In some embodiments, the R³ groups are geminal methyl and cyclopropyl groups. In some embodiments, the R³ groups are geminal methoxymethyl and cyclopropyl groups. In some embodiments, m is 2, one R³ is trifluoromethyl and the other R³ is cyclopropyl. In some embodiments, the R³ groups are geminal trifluoromethyl and cyclopropyl groups.

In some embodiments, m is 2 and the two R³ together with the carbon atom to which they are attached come together to form an oxo group. In some embodiments, m is 2 and the two R³ together to form a C3-C8 cycloalkyl (e.g., a cyclopropyl, cyclobutyl, cyclopentyl, cyclohexyl, cycloheptyl, or cyclooctyl).

In some embodiments, R⁴ is phenyl, napthyl, 5-10 membered heteroaryl, 3-10 membered heterocyclyl, or C3-C8 cycloalkyl; wherein each R⁴ group is optionally substituted with 1-2 independently selected R⁶. In some embodiments, R⁴ is phenyl, 5-6 membered heteroaryl, 3-10 membered heterocyclyl, or C3-C8 cycloalkyl; wherein each R⁴ group is optionally substituted with 1-2 independently selected R⁶. In some embodiments, R⁴ is phenyl, naphthyl, 5-10 membered heteroaryl, 3-10 membered heterocyclyl, or C3-C8 cycloalkyl; wherein each R⁴ group is optionally substituted with 2-3 independently selected R⁶. In some embodiments, R⁴ is phenyl, naphthyl, 5-10 membered heteroaryl, 3-10 membered heterocyclyl, or C3-C8 cycloalkyl; wherein each R⁴ group is optionally substituted with 1 or 3 independently selected R⁶. In some embodiments, R⁴ is phenyl, naphthyl, 5-10 membered heteroaryl, 3-10 membered heterocyclyl, or C3-C8 cycloalkyl; wherein each R⁴ group is optionally substituted with 1 independently selected R⁶. In some embodiments, R⁴ is phenyl, naphthyl, 5-10 membered heteroaryl, 3-10 membered heterocyclyl, or C3-C8 cycloalkyl; wherein each R⁴ group is optionally substituted with 2 independently selected R⁶. In some embodiments, R⁴ is phenyl, naphthyl, 5-10 membered heteroaryl, 3-10 membered heterocyclyl, or C3-C8 cycloalkyl; wherein each R⁴ group is optionally substituted with 3 independently selected R⁶. In some embodiments, R⁴ is phenyl, 5-6 membered heteroaryl, 3-10 membered heterocyclyl, or C3-C8 cycloalkyl; wherein each R⁴ group is optionally substituted with 2-3 independently selected R⁶. In some embodiments, R⁴ is phenyl, 5-6 membered heteroaryl, 3-10 membered heterocyclyl, or C3-C8 cycloalkyl; wherein each R⁴ group is optionally substituted with 1 or 3 independently selected R⁶. In some embodiments, R⁴ is phenyl, 5-6 membered heteroaryl, 3-10 membered heterocyclyl, or C3-C8 cycloalkyl; wherein each R⁴ group is optionally substituted with 1 independently selected R⁶. In some embodiments, R⁴ is phenyl, 5-6 membered heteroaryl, 3-10 membered heterocyclyl, or C3-C8 cycloalkyl; wherein each R⁴ group is optionally substituted with 2 independently selected R⁶. In some embodiments, R⁴ is phenyl, 5-6 membered heteroaryl, 3-10 membered heterocyclyl, or C3-C8 cycloalkyl; wherein each R⁴ group is optionally substituted with 3 independently selected R⁶.

In some embodiments, R⁴ is phenyl or 5 membered heteroaryl; wherein each R⁴ group is optionally substituted with 1-3 substituents independently selected from R⁶. In some embodiments, R⁴ is phenyl or 6 membered heteroaryl; wherein each R⁴ group is optionally substituted with 1-3 substituents independently selected from R⁶. In some embodiments, R⁴ is naphthyl or 9-10 membered heteroaryl; wherein each R⁴ group is optionally substituted with 1-3 substituents independently selected from R⁶.

In some embodiments, R⁴ is phenyl, 5 membered heteroaryl, or cyclopentyl; wherein each R⁴ group is optionally substituted with 1-3 substituents independently selected from R⁶. In some embodiments, R⁴ is phenyl, 6 membered heteroaryl, cyclopentyl, or cyclohexyl; wherein each R⁴ group is optionally substituted with 1-3 substituents independently selected from R⁶.

In some embodiments, R⁴ is phenyl optionally substituted with 1-3 independently selected R⁶. In certain embodiments, R⁴ is phenyl optionally substituted with 1 R⁶. In certain embodiments, R⁴ is phenyl optionally substituted with 2 independently selected R⁶. In certain embodiments, R⁴ is phenyl optionally substituted with 3 independently selected R⁶.

In some embodiments, R⁴ is unsubstituted phenyl.

In some embodiments, R⁴ is phenyl substituted with 1-3 substituents independently selected from R⁶. In certain embodiments, R⁴ is phenyl substituted with R⁶. In certain embodiments, R⁴ is phenyl substituted with 2 independently selected R⁶. In some embodiments, R⁴ is phenyl substituted with 3 independently selected R⁶.

In some embodiments, R⁴ is naphthyl optionally substituted with 1-3 independently selected R⁶. In some embodiments, R⁴ is naphthyl substituted with 1-3 independently selected R⁶.

In some embodiments, R⁴ is unsubstituted naphthyl.

In some embodiments, R⁴ is 5-6 membered heteroaryl optionally substituted with 1-3 (e.g., 2) substituents independently selected from R⁶. In some embodiments, R⁴ is 6 membered heteroaryl optionally substituted with 1-3 (e.g., 2) independently selected R⁶. In some embodiments, R⁴ is 9-10 membered heteroaryl optionally substituted with 1-3 (e.g., 2) independently selected R⁶. In some embodiments, R⁴ is 9 membered heteroaryl optionally substituted with 1-3 (e.g., 2) independently selected R⁶. In some embodiments, R⁴ is 10 membered heteroaryl optionally substituted with 1-3 (e.g., 2) independently selected R⁶.

In some embodiments, R⁴ is unsubstituted 5-6 membered heteroaryl. In some embodiments, R⁴ is unsubstituted 9-10 membered heteroaryl.

In some embodiments, R⁴ is 5-6 membered heteroaryl substituted with 1-3 substituents independently selected from R⁶. In some embodiments, R⁴ is 9-10 membered heteroaryl substituted with 1-3 substituents independently selected from R⁶.

In some embodiments, the 5-6 membered heteroaryl is 3-pyridyl, 4-pyridyl, or 4-pyridazinyl. In some embodiments, the R⁴ 5-6 membered heteroaryl is 3-pyridyl or 4-pyridyl. In some embodiments, the R⁴ 5-6 membered heteroaryl is pyridonyl.

In some embodiments, R⁴ is 3-10 membered heterocyclyl optionally substituted with 1-3 substituents independently selected from R⁶. In some embodiments, R⁴ is 6-10 membered heterocyclyl optionally substituted with 1-3 substituents independently selected from R⁶. In some embodiments, R⁴ is 3-10 membered heterocyclyl substituted with 1-3 substituents independently selected from R⁶. In some embodiments, R⁴ is 6-10 membered heterocyclyl substituted with 1-3 substituents independently selected from R⁶. In some embodiments, R⁴ is 3-10 membered heterocyclyl substituted with 1-2 substituents independently selected from R⁶. In some embodiments, R⁴ is 6-10 membered heterocyclyl substituted with 1-2 substituents independently selected from R⁶. In some embodiments, R⁴ is 3-10 membered heterocyclyl. In some embodiments, R⁴ is 6-10 membered heterocyclyl. In some embodiments, R⁴ is morpholino, optionally substituted with 1-2 independently selected R⁶. In some embodiments, R⁴ is tetrahydropyranyl, optionally substituted with 1-2 independently selected R⁶. In some embodiments, R⁴ is 1-oxaspiro[4.5]decane, optionally substituted with 1-2 independently selected R⁶.

In some embodiments, R⁴ is C3-C8 cycloalkyl optionally substituted with 1-3 independently selected R⁶. In certain embodiments, R⁴ is C3-C8 cycloalkyl optionally substituted with 1 R⁶. In certain embodiments, R⁴ is C3-C8 cycloalkyl optionally substituted with 2 independently selected R⁶. In certain embodiments, R⁴ is C3-C8 cycloalkyl optionally substituted with 3 independently selected R⁶.

In some embodiments, R⁴ is unsubstituted C3-C8 cycloalkyl.

In some embodiments, R⁴ is C3-C8 cycloalkyl substituted with 1-3 independently selected R⁶. In certain embodiments, R⁴ is C3-C8 cycloalkyl substituted with 1 R⁶. In certain embodiments, R⁴ is C3-C8 cycloalkyl substituted with 2 independently selected R⁶. In certain embodiments, R⁴ is C3-C8 cycloalkyl substituted with 3 independently selected R⁶.

In some embodiments, at least one of R⁶ is halogen. In some embodiments, at least one of R⁶ is fluoro. In some embodiments, at least one of R⁶ is chloro. In some embodiments, one of R⁶ is halogen. In some embodiments, one of R⁶ is fluoro. In some embodiments, one of R⁶ is chloro. In some embodiments, two of R⁶ is halogen. In some embodiments, two of R⁶ is fluoro. In some embodiments, two of R⁶ is chloro. In some embodiments, three of R⁶ is halogen. In some embodiments, three of R⁶ is fluoro. In some embodiments, three of R⁶ is chloro. In some embodiments, at least one of R⁶ is cyano. In some embodiments, at least one of R⁶ is hydroxyl. In some embodiments, at least one of R⁶ is —CO₂H.

In some embodiments, at least one of R⁶ is —N═(S═O)(C1-C3 alkyl)₂. For example, at least one of R⁶ is —N═(S═O)(methyl)₂.

In some embodiments, at least one of R⁶ is —S(═O)ₚ(C1-C3 alkyl) (e.g., —S(═O)ₚ(methyl)). In some embodiments, at least one of R⁶ is —S(═O)(C1-C3 alkyl) (e.g., —S(═O)(methyl)). In some embodiments, at least one of R⁶ is —S(═O)₂(C1-C3 alkyl) (e.g., —S(═O)₂(methyl)).

In some embodiments, p is 1. In some embodiments, p is 2.

In some embodiments, at least one of R⁶ is —NR^{E}R^{F}. In some embodiments, at least one of R⁶ is —(C═O)NR^{E}R^{F}.

In some embodiments, at least one of R⁶ is C1-C3 alkoxy optionally substituted with amino, hydroxyl, or —(C═O)NR^{E}R^{F}. In some embodiments, at least one of R⁶ is unsubstituted C1-C3 alkoxy. In some embodiments, at least one of R⁶ is C1-C3 alkoxy substituted with amino, hydroxyl, or —(C═O)NR^{E}R^{F}. In some embodiments, at least one of R⁶ is C1-C3 alkoxy substituted with amino. In some embodiments, at least one of R⁶ is C1-C3 alkoxy substituted with hydroxyl. In some embodiments, at least one of R⁶ is C1-C3 alkoxy substituted with —(C═O)NR^{E}R^{F}.

In certain embodiments, at least one of R⁶ is methoxy or ethoxy.

In some embodiments, R^{E} and R^{F} are independently hydrogen or C1-C3 alkyl. In certain embodiments, one of R^{E} and R^{F} is hydrogen and the other of R^{E} and R^{F} is C1-C3 alkyl. In some embodiments, one of R^{E} and R^{F} is hydrogen and the other of R^{E} and R^{F} is methyl. In some embodiments, one of R^{E} and R^{F} is hydrogen and the other of R^{E} and R^{F} is ethyl. In certain embodiments, R^{E} and R^{F} are both hydrogens. In certain embodiments, R^{E} and R^{F} are both C1-C3 alkyl. In some embodiments, R^{E} and R^{F} are both methyl. In some embodiments, one of R^{E} and R^{F} is methyl and the other of R^{E} and R^{F} is ethyl. In some embodiments, R^{E} and R^{F} are both ethyl. In some embodiments, R^{E} and R^{F} are independently hydrogen or C3-C6 cycloalkyl. In some embodiments, R^{E} and R^{F} are independently hydrogen or cyclopropyl. In some embodiments, one of R^{E} and R^{F} is hydrogen and the other of R^{E} and R^{F} is cyclopropyl.

In some embodiments, R^{E} and R^{F} together with the nitrogen atom to which they are attached come together to form a 4-6 membered heterocyclyl optionally substituted with C1-C3 alkyl or C1-C3 alkoxy. In certain embodiments, R^{E} and R^{F} together with the nitrogen atom to which they are attached come together to form a 4 membered heterocyclyl optionally substituted with C1-C3 alkyl or C1-C3 alkoxy. In some embodiments, R^{E} and R^{F} together with the nitrogen atom to which they are attached come together to form a 5 membered heterocyclyl optionally substituted with C1-C3 alkyl or C1-C3 alkoxy. In some embodiments, R^{E} and R^{F} together with the nitrogen atom to which they are attached come together to form a 6 membered heterocyclyl optionally substituted with C1-C3 alkyl or C1-C3 alkoxy. In some embodiments, R^{E} and R^{F} together with the nitrogen atom to which they are attached come together to form a 4-6 membered heterocyclyl substituted with C1-C3 alkyl or C1-C3 alkoxy. In some embodiments, R^{E} and R^{F} together with the nitrogen atom to which they are attached come together to form a 4-6 membered heterocyclyl substituted with C1-C3 alkyl. In some embodiments, R^{E} and R^{F} together with the nitrogen atom to which they are attached come together to form a 4-6 membered heterocyclyl substituted with C1-C3 alkoxy. In some embodiments, R^{E} and R^{F} together with the nitrogen atom to which they are attached come together to form an unsubstituted 4-6 membered heterocyclyl.

In some embodiments, at least one of R⁶ is C1-C3 haloalkyl. In certain embodiments, at least one of R⁶ is trifluoromethyl, difluoromethyl, or 2,2,2-trifluoroethyl. In certain embodiments, at least one of R⁶ is trifluoromethyl or 2,2,2-trifluoroethyl. In some embodiments, at least one of R⁶ is difluoromethyl.

In some embodiments, at least one of R⁶ is C1-C3 haloalkoxy. In some embodiments, at least one of R⁶ is trifluoromethoxy. In some embodiments, at least one of R⁶ is difluoromethoxy.

In some embodiments, at least one of R⁶ is 5-6 membered heteroaryl optionally substituted with 1-3 independently selected R^{X}. In some embodiments, at least one of R⁶ is 5-6 membered heteroaryl optionally substituted with 1-2 independently selected R^{X}. In some embodiments, at least one of R⁶ is 5-6 membered heteroaryl optionally substituted with 2-3 independently selected R^{X}. In some embodiments, at least one of R⁶ is 5-6 membered heteroaryl optionally substituted with 1 or 3 independently selected R^{X}. In some embodiments, at least one of R⁶ is 5-6 membered heteroaryl optionally substituted with 1 R^{X}. In some embodiments, at least one of R⁶ is 5-6 membered heteroaryl optionally substituted with 2 independently selected R^{X}. In some embodiments, at least one of R⁶ is 5-6 membered heteroaryl optionally substituted with 3 independently selected R^{X}. In some embodiments, at least one of R⁶ is 5-6 membered heteroaryl optionally substituted with halogen, cyano, hydroxyl, C1-C3 alkoxy, C1-C3 haloalkoxy, amino, C1-C3 haloalkyl, or C1-C3 alkyl optionally substituted with hydroxyl or —NR^{E}R^{F}. In some embodiments, R⁶ is 5-6 membered heteroaryl optionally substituted with C1-C3 alkyl optionally substituted with hydroxyl or —NR^{E}R^{F}. In some embodiments, at least one of R⁶ is 5-6 membered heteroaryl optionally substituted with halogen, C1-C3 haloalkyl, or C1-C3 alkyl optionally substituted with hydroxyl or —NR^{E}R^{F}. In some embodiments, R⁶ is 5-6 membered heteroaryl substituted with C1-C3 alkyl substituted with hydroxyl or —NR^{E}R^{F}. In some embodiments, R⁶ is 5-6 membered heteroaryl substituted with hydroxymethyl, aminomethyl, hydroxyethyl, aminoethyl, propan-2-ol, or propan-2-amine.

In certain embodiments, at least one of R⁶ is 5 membered heteroaryl optionally substituted with 1-3 (e.g., 1-2, 2-3, 1, 2, or 3) independently selected R^{X}. In certain embodiments, at least one of R⁶ is 5 membered heteroaryl optionally substituted with halogen, cyano, hydroxyl, C1-C3 alkoxy, C1-C3 haloalkoxy, C1-C3 alkyl, amino, or C1-C3 haloalkyl. In some embodiments, at least one of R⁶ is 6 membered heteroaryl optionally substituted with halogen, cyano, hydroxyl, C1-C3 alkoxy, C1-C3 haloalkoxy, amino, C1-C3 haloalkyl, or C1-C3 alkyl optionally substituted with hydroxyl or —NR^{E}R^{F}. In some embodiments, R⁶ is 5 membered heteroaryl substituted with hydroxymethyl, aminomethyl, hydroxyethyl, aminoethyl, propan-2-ol, or propan-2-amine. In some embodiments, R⁶ is 6 membered heteroaryl substituted with hydroxymethyl, aminomethyl, hydroxyethyl, aminoethyl, propan-2-ol, or propan-2-amine.

In some embodiments, at least one of R⁶ is unsubstituted 5-6 membered heteroaryl. In some embodiments, at least one of R⁶ is 1,2,3-triazol-2-yl.

In some embodiments, each R^{X} is independently selected from cyano, hydroxyl, C1-C3 alkoxy, or C1-C6 alkyl optionally substituted with 1-3 substituents independently selected from hydroxyl, C1-C3 alkoxy, and —NR^{G}R^{H}. In some embodiments, each R^{X} is independently selected from hydroxyl or C1-C6 alkyl optionally substituted with 1-3 substituents independently selected from hydroxyl, C1-C3 alkoxy, and —NR^{G}R^{H}. In some embodiments, each R^{X} is independently selected from hydroxyl or C1-C2 alkyl optionally substituted with 1-3 (e.g., 1-2) substituents independently selected from hydroxyl, methoxy, and dimethylamino. In some embodiments, each R^{X} is independently selected from hydroxyl or C1-C4 alkyl optionally substituted with 1-3 substituents independently selected from hydroxyl, C1-C3 alkoxy, and —NR^{G}R^{H}.

In some embodiments, R^{G} and R^{H} are independently hydrogen or C1-C3 alkyl. In certain embodiments, one of R^{G} and R^{H} is hydrogen and the other of R^{G} and R^{H} is C1-C3 alkyl. In some embodiments, one of R^{G} and R^{H} is hydrogen and the other of R^{G} and R^{H} is methyl. In some embodiments, one of R^{G} and R^{H} is hydrogen and the other of R^{G} and R^{H} is ethyl. In certain embodiments, R^{G} and R^{H} are both hydrogens. In certain embodiments, R^{G} and R^{H} are both C1-C3 alkyl. In some embodiments, R^{G} and R^{H} are both methyl. In some embodiments, one of R^{G} and R^{H} is methyl and the other of R^{G} and R^{H} is ethyl. In some embodiments, R^{G} and R^{H} are both ethyl. In some embodiments, R^{G} and R^{H} are independently hydrogen or C3-C6 cycloalkyl. In some embodiments, R^{G} and R^{H} are independently hydrogen or cyclopropyl. In some embodiments, one of R^{G} and R^{H} is hydrogen and the other of R^{G} and R^{H} is cyclopropyl.

In some embodiments, R^{G} and R^{H} together with the nitrogen atom to which they are attached come together to form a 4-6 membered heterocyclyl optionally substituted with C1-C3 alkyl or C1-C3 alkoxy. In certain embodiments, R^{G} and R^{H} together with the nitrogen atom to which they are attached come together to form a 4 membered heterocyclyl optionally substituted with C1-C3 alkyl or C1-C3 alkoxy. In some embodiments, R^{G} and R^{H} together with the nitrogen atom to which they are attached come together to form a 5 membered heterocyclyl optionally substituted with C1-C3 alkyl or C1-C3 alkoxy. In some embodiments, R^{G} and R^{H} together with the nitrogen atom to which they are attached come together to form a 6 membered heterocyclyl optionally substituted with C1-C3 alkyl or C1-C3 alkoxy. In some embodiments, R^{G} and R^{H} together with the nitrogen atom to which they are attached come together to form a 4-6 membered heterocyclyl substituted with C1-C3 alkyl or C1-C3 alkoxy. In some embodiments, R^{G} and R^{H} together with the nitrogen atom to which they are attached come together to form a 4-6 membered heterocyclyl substituted with C1-C3 alkyl. In some embodiments, R^{G} and R^{H} together with the nitrogen atom to which they are attached come together to form a 4-6 membered heterocyclyl substituted with C1-C3 alkoxy. In some embodiments, R^{G} and R^{H} together with the nitrogen atom to which they are attached come together to form an unsubstituted 4-6 membered heterocyclyl.

In some embodiments, at least one of R⁶ is C1-C3 alkyl optionally substituted with 1-2 substituents independently selected from hydroxyl, —NR^{E}R^{F}, C1-C3 alkoxy, and C3-C6 cycloalkyl. In some embodiments, at least one of R⁶ is C3-C6 cycloalkyl optionally substituted with hydroxyl.

In some embodiments, at least one of R⁶ is C1-C3 alkyl optionally substituted with hydroxyl, —NR^{E}R^{F}, or C1-C3 alkoxy. In certain embodiments, at least one of R⁶ is methyl optionally substituted with hydroxyl, —NR^{E}R^{F}, or C1-C3 alkoxy. In some embodiments, at least one of R⁶ is hydroxymethyl, 2-aminoethyl, or methoxyethyl. In some embodiments, at least one of R⁶ is ethyl optionally substituted with hydroxyl, —NR^{E}R^{F}, or C1-C3 alkoxy.

In some embodiments, R^{E} and R^{F} together with the nitrogen atom to which they are attached come together to form a 4-6 membered heterocyclyl optionally substituted with C1-C3 alkyl or C1-C3 alkoxy. In certain embodiments, R^{E} and R^{F} together with the nitrogen atom to which they are attached come together to form a 4 membered heterocyclyl. In some embodiments, R^{E} and R^{F} together with the nitrogen atom to which they are attached come together to form a 5 membered heterocyclyl. In some embodiments, R^{E} and R^{F} together with the nitrogen atom to which they are attached come together to form a 6 membered heterocyclyl. In some embodiments, R^{E} and R^{F} together with the nitrogen atom to which they are attached come together to form a 4-6 membered heterocyclyl substituted with C1-C3 alkyl or C1-C3 alkoxy. In some embodiments, R^{E} and R^{F} together with the nitrogen atom to which they are attached come together to form an unsubstituted 4-6 membered heterocyclyl.

In some embodiments, at least one of R⁶ is -(Q)_{q}-3-8 membered heterocyclyl optionally substituted with 1-3 independently selected C1-C3 alkyl. In some embodiments, at least one of R⁶ is —O-3-8 membered heterocyclyl optionally substituted with 1-3 independently selected C1-C3 alkyl. In some embodiments, at least one of R⁶ is —NH-3-8 membered heterocyclyl optionally substituted with 1-3 independently selected C1-C3 alkyl. In some embodiments, R⁶ is -(Q)_{q}-3-8 membered heterocyclyl. In some embodiments, R⁶ is -(Q)_{q}-3-8 membered heterocyclyl substituted with 1-3 independently selected C1-C3 alkyl. In some embodiments, R⁶ is -(Q)_{q}-3-8 membered heterocyclyl substituted with C1-C3 alkyl. In some embodiments, R⁶ is -(Q)_{q}-3-8 membered heterocyclyl substituted with 2 independently selected C1-C3 alkyl. In some embodiments, R⁶ is -(Q)_{q}-3-8 membered heterocyclyl substituted with 3 independently selected C1-C3 alkyl.

In some embodiments, q is 0. In some embodiments, q is 1.

In some embodiments, Q is —O—. In some embodiments, Q is-NH—.

In some embodiments, at least one of R⁶ is 3-8 membered heterocyclyl. In certain embodiments, at least one of R⁶ is 3 membered heterocyclyl. In certain embodiments, at least one of R⁶ is 4 membered heterocyclyl. In certain embodiments, at least one of R⁶ is 5 membered heterocyclyl. In certain embodiments, at least one of R⁶ is 5 membered heterocyclyl comprising 1 heteroatom ring member selected from O, S, and NH. In certain embodiments, at least one of R⁶ is tetrahydrofuranyl (e.g., 2-tetrahydrofuranyl). In certain embodiments, at least one of R⁶ is 6 membered heterocyclyl. In certain embodiments, at least one of R⁶ is 7 membered heterocyclyl. In certain embodiments, at least one of R⁶ is 8 membered heterocyclyl.

In some embodiments, R⁴ is pyridyl, pyrimidinyl, pyrazinyl, pyrrolyl, or imidazolyl; each of which is substituted with 2 R⁶: one R⁶ is triazolyl, imidazolyl, oxazolyl, pyrazolyl, or pyrrolidinyl; and the other R⁶ is methoxy, trifluoromethyl, trifluoromethoxy, chloro, or cyano. In some embodiments, R⁴ is pyridyl, pyrimidinyl, or pyrazinyl; each of which is substituted with 2 R⁶: one R⁶ is triazolyl, imidazolyl, oxazolyl, pyrazolyl, or pyrrolidinyl; and the other R⁶ is methoxy, trifluoromethyl, trifluoromethoxy, chloro, or cyano. In some embodiments, R⁴ is pyridyl substituted with 2 R⁶: one R⁶ is triazolyl, imidazolyl, or oxazolyl; and the other R⁶ is methoxy, trifluoromethyl, trifluoromethoxy, chloro, or cyano. In some embodiments, R⁴ is pyridyl or phenyl; each of which is substituted with 2 R⁶: one R⁶ is triazolyl or pyrazolyl, each optionally substituted with hydroxymethyl, methyl, hydroxyl, hydroxyethyl, cyano, or methoxy; and the other R⁶ is methoxy, trifluoromethyl, trifluoromethoxy, difluoromethyl, chloro, or cyano.

In some embodiments, R⁴ is 3-pyridyl or 4-pyridyl substituted with 1-3 independently selected R⁶.

In some embodiments, R⁴ is

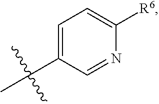

wherein the wavy line crosses the bond that connects to the —C(═O)NH— moiety of Formula (I).

In some embodiments, R⁴ is

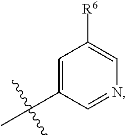

wherein the wavy line crosses the bond that connects to the —C(═O)NH— moiety of Formula (I).

In some embodiments, R⁴ is

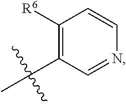

wherein the wavy line crosses the bond that connects to the —C(═O)NH— moiety of Formula (I).

In some embodiments, R⁴ is

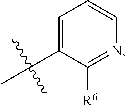

wherein the wavy line crosses the bond that connects to the —C(═O)NH— moiety of Formula (I).

In some embodiments, R⁴ is

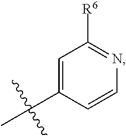

wherein the wavy line crosses the bond that connects to the —C(═O)NH— moiety of Formula (I).

In some embodiments, R⁴ is

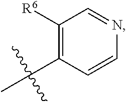

wherein the wavy line crosses the bond that connects to the —C(═O)NH— moiety of Formula (I).

In some embodiments, when R⁴ is

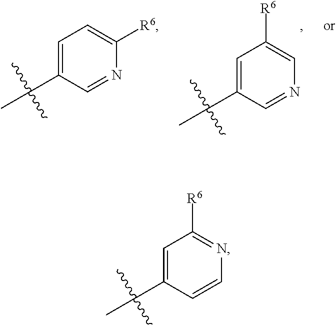

R⁶ is selected from the group consisting of cyano, halogen, C1-C3 haloalkyl, and C1-C3 alkoxy.

In some embodiments, when R⁴ is

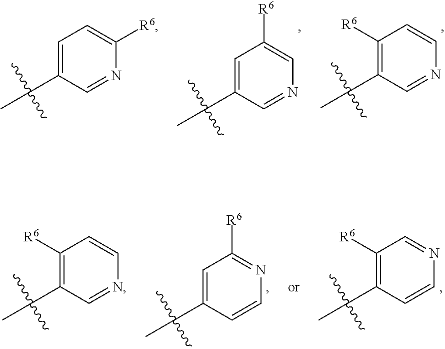

R⁶ is selected from the group consisting of cyano, halogen, C1-C3 haloalkyl, and C1-C3 alkoxy.

In some embodiments, when R⁴ is

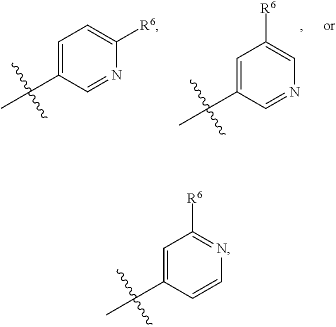

R⁶ is selected from the group consisting of cyano, chloro, difluoromethyl, trifluoromethyl, and methoxy. For example, when R⁴ is

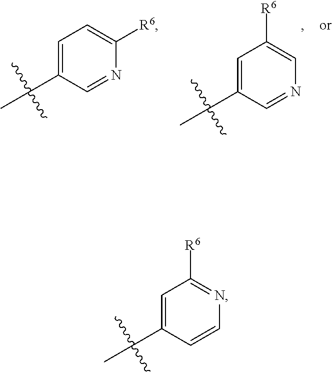

R⁶ is chloro or trifluoromethyl (e.g., chloro).

In some embodiments, R⁴ is

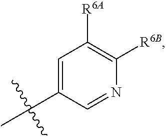

wherein the the wavy line crosses the bond that connects to the —C(═O)NH— moiety of Formula (I).

In some embodiments, R⁴ is

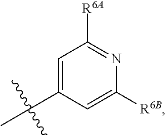

wherein the the wavy line crosses the bond that connects to the —C(═O)NH— moiety of Formula (I).

In some embodiments, R⁴ is

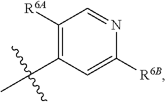

wherein the the wavy line crosses the bond that connects to the —C(═O)NH— moiety of Formula (I).

In some embodiments, when R⁴ is

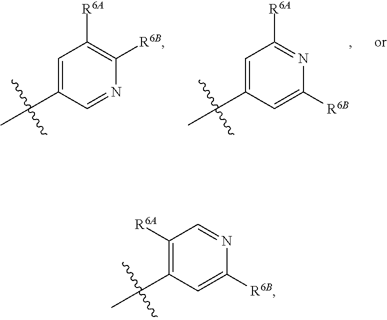

R^{6A} is selected from the group consisting of: cyano, halogen, C1-C3 alkyl, C1-C3 alkoxy, and C1-C3 haloalkyl; and
R^{6B} is selected from the group consisting of: 5-6 membered heteroaryl optionally substituted with cyano, amino, or C1-C3 alkyl optionally substituted with hydroxyl or —NR^{E}R^{F}; —(C═O)NR^{E}R^{F}; C1-C3 alkoxy; C1-C3 haloalkyl; C1-C3 haloalkoxy; cyano; and C1-C3 alkyl.

In some embodiments, when R⁴ is

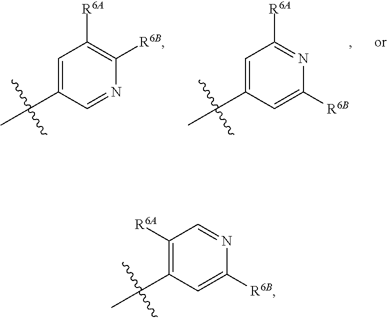

R^{6A} is selected from the group consisting of: cyano, halogen, unsubstituted C1-C3 alkyl, C1-C3 alkoxy, and C1-C3 haloalkyl; and
R^{6B} is selected from the group consisting of: 5-6 membered heteroaryl optionally substituted with cyano, hydroxyl, —N═(S═O)(C1-C3 alkyl)₂, C1-C3 alkoxy, C1-C3 alkyl optionally substituted with 1-2 substituents independently selected from hydroxyl, C1-C3 alkoxy, and —NR^{G}R^{H}, or amino; —(C═O)NR^{E}R^{F}; C1-C3 alkoxy; C1-C3 haloalkyl; C1-C3 haloalkoxy; cyano; C1-C3 alkyl; and -(Q)_{q}-3-8 membered heterocyclyl optionally substituted with 1-3 independently selected C1-C3 alkyl.

In some embodiments, when R⁴ is

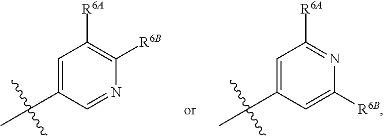

R^{6A} is selected from the group consisting of: cyano, fluoro, chloro, methyl, ethyl, methoxy, trifluoromethyl; and
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, 1,2,4-triazol-4-on-2-yl, tetrazol-5-yl, 2-methyl-tetrazol-5-yl, 1-methyl-tetrazol-5-yl, imidazol-1-yl, 1-methyl-imidazol-3-yl, 1-methyl-5-amino-imidazol-3-yl, 3-methylimidazol-2-on-1-yl, 1-methyl-pyrazol-3-yl, 1-methyl-pyrazol-5-yl, pyrrol-1-yl, thiazol-2-yl, isothiazolidin-2-yl-1,1-dioxide, pyrrolidin-2-on-1-yl, oxazol-2-yl, oxadiazol-2-yl, 2-amino-pyrimidin-4-yl, —(C═O)4-methylpiperazin-1-yl, —(C═O)N(CH₃)₂, —(C═O)NHCH₃, methoxy, ethoxy, difluoromethoxy, methyl, cyano.

In some embodiments, when R⁴ is

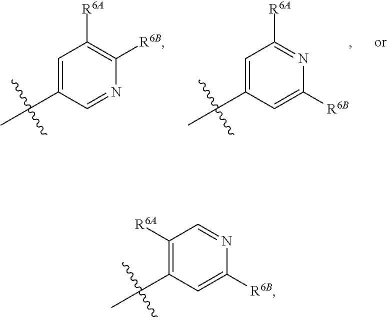

R^{6A} is selected from the group consisting of: cyano, fluoro, chloro, methyl, ethyl, methoxy, trifluoromethyl; and
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, 1,2,4-triazol-4-on-2-yl, tetrazol-5-yl, 2-methyl-tetrazol-5-yl, 1-methyl-tetrazol-5-yl, imidazol-1-yl, 1-methyl-imidazol-3-yl, 1-methyl-5-amino-imidazol-3-yl, 3-methylimidazol-2-on-1-yl, 1-methyl-pyrazol-3-yl, 1-methyl-pyrazol-5-yl, pyrrol-1-yl, thiazol-2-yl, isothiazolidin-2-yl-1,1-dioxide, pyrrolidin-2-on-1-yl, oxazol-2-yl, oxadiazol-2-yl, 2-amino-pyrimidin-4-yl, —(C═O)4-methylpiperazin-1-yl, —(C═O)N(CH₃)₂, —(C═O)NHCH₃, methoxy, ethoxy, difluoromethoxy, trifluoromethyl, methyl, and cyano.

In some embodiments, when R⁴ is

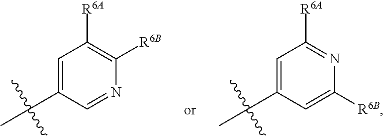

R^{6A} is selected from the group consisting of: cyano, fluoro, chloro, methyl, ethyl, methoxy, difluoromethyl, trifluoromethyl; and
R^{6B} is selected from the group consisting of: 1,2,3-triazol-2-yl, 4-methyl-1,2,3-triazol-2-yl, 4-hydroxymethyl-1,2,3-triazol-2-yl, 4-(1,2-dihydroxyethyl)-1,2,3-triazol-2-yl, 4-(1-hydroxyethyl)-1,2,3-triazol-2-yl, 4-methoxymethyl-1,2,3-triazol-2-yl, 4-methyl-1,2,3-triazol-1-yl, 4-methoxy-1,2,3-triazol-2-yl, 4-amino-1,2,3-triazol-2-yl, 4-dimethylaminomethyl-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, 1,2,4-triazol-4-on-2-yl, tetrazol-5-yl, 2-methyl-tetrazol-5-yl, 1-methyl-tetrazol-5-yl, imidazol-1-yl, pyrazol-1-yl, 5-cyano-pyrazol-1-yl, 1-methyl-imidazol-3-yl, 1-methyl-5-amino-imidazol-3-yl, 3-methylimidazol-2-on-1-yl, 1-methyl-pyrazol-3-yl, 1-methyl-pyrazol-5-yl, pyrrol-1-yl, thiazol-2-yl, isothiazolidin-2-yl-1,1-dioxide, pyrrolidin-2-on-1-yl, oxazol-2-yl, oxadiazol-2-yl, 2-amino-pyrimidin-4-yl, 2-tetrahydrofuranyl, —(C═O)4-methylpiperazin-1-yl, —(C═O)N(CH₃)₂, —(C═O)NHCH₃, —N═(S═O)(methyl)₂, methoxy, ethoxy, difluoromethoxy, methyl, cyano.

In some embodiments, when R⁴ is

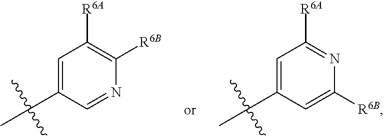

R^{6A} is selected from the group consisting of: cyano, chloro, and trifluoromethyl; and
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, and 1,2,4-triazol-4-on-2-yl.

In some embodiments, when R⁴ is

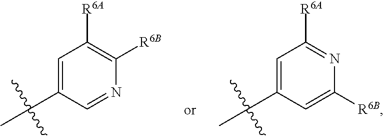

R^{6A} is chloro; and
R^{6B} is selected from the group consisting of: 1,2,3-triazol-2-yl, 1,2,3-triazol-1-yl, and 1,2,4-triazol-4-on-2-yl.

In some embodiments, R⁴ is

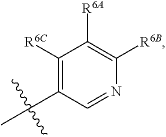

wherein the the wavy line crosses the bond that connects to the —C(═O)NH— moiety of Formula (I).

In some embodiments, R⁴ is

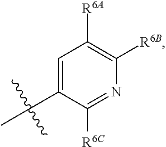

wherein the the wavy line crosses the bond that connects to the —C(═O)NH— moiety of Formula (I).

In some embodiments, R⁴ is

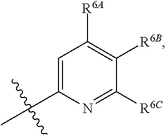

wherein the the wavy line crosses the bond that connects to the —C(═O)NH— moiety of Formula (I).

In some embodiments, when R⁴ is

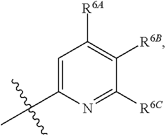

R^{6A} is selected from the group consisting of: cyano, halogen, C1-C3 alkyl, C1-C3 alkoxy, and C1-C3 haloalkyl;
R^{6B} is selected from the group consisting of: 5-6 membered heteroaryl optionally substituted with cyano, C1-C3 alkyl, or amino; —(C═O)NR^{E}R^{F}; C1-C3 alkoxy; C1-C3 haloalkyl; C1-C3 haloalkoxy; cyano; and C1-C3 alkyl; and
R^{6C} is selected from the group consisting of: cyano, halogen, C1-C3 alkyl, C1-C3 alkoxy, C1-C3 haloalkyl, and -(Q)_{q}-3-8 membered heterocyclyl optionally substituted with 1-3 independently selected C1-C3 alkyl.

In some embodiments, when R⁴ is

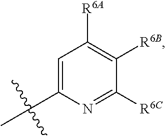

R^{6A} is selected from the group consisting of: cyano, fluoro, chloro, methyl, ethyl, methoxy, trifluoromethyl;
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, 1,2,4-triazol-4-on-2-yl, tetrazol-5-yl, 2-methyl-tetrazol-5-yl, 1-methyl-tetrazol-5-yl, imidazol-1-yl, 1-methyl-imidazol-3-yl, 1-methyl-5-amino-imidazol-3-yl, 3-methylimidazol-2-on-1-yl, 1-methyl-pyrazol-3-yl, 1-methyl-pyrazol-5-yl, pyrrol-1-yl, thiazol-2-yl, isothiazolidin-2-yl-1,1-dioxide, pyrrolidin-2-on-1-yl, oxazol-2-yl, oxadiazol-2-yl, 2-amino-pyrimidin-4-yl, —(C═O)4-methylpiperazin-1-yl, —(C═O)N(CH₃)₂, —(C═O)NHCH₃, methoxy, ethoxy, difluoromethoxy, methyl, cyano; and
R^{6C} is selected from the group consisting of: cyano, fluoro, chloro, methyl, ethyl, methoxy, methyl, trifluoromethyl, and pyrrolidin-3-yloxy.

In some embodiments, when R⁴ is

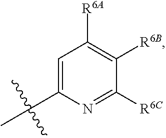

R^{6A} is selected from the group consisting of: cyano, chloro, and trifluoromethyl;
R^{6B} is selected from the group consisting of: methoxy, 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, and 1,2,4-triazol-4-on-2-yl; and
R^{6C} is selected from the group consisting of: cyano, chloro, methyl, trifluoromethyl, and pyrrolidin-3-yloxy.

In some embodiments, when R⁴ is

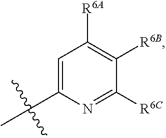

R^{6A} is chloro;
R^{6B} is selected from the group consisting of: methoxy, 1,2,3-triazol-2-yl, 1,2,4-triazol-4-on-2-yl; and
R^{6C} is selected from the group consisting of: cyano, chloro, methyl, trifluoromethyl, and pyrrolidin-3-yloxy.

In some embodiments, when R⁴ is

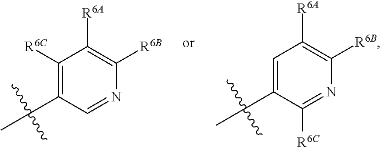

R^{6A} is selected from the group consisting of: cyano, halogen, C1-C3 alkyl, C1-C3 alkoxy, and C1-C3 haloalkyl;
R^{6B} is selected from the group consisting of: 5-6 membered heteroaryl optionally substituted with cyano, C1-C3 alkyl, or amino; —(C═O)NR^{E}R^{F}; C1-C3 alkoxy; C1-C3 haloalkyl; C1-C3 haloalkoxy; cyano; and C1-C3 alkyl; and
R^{6C} is selected from the group consisting of: cyano, halogen, C1-C3 alkyl, C1-C3 alkoxy, and C1-C3 haloalkyl.

In some embodiments, when R⁴ is

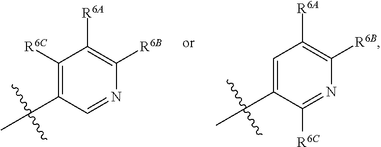

R^{6A} is selected from the group consisting of: cyano, fluoro, chloro, methyl, ethyl, methoxy, trifluoromethyl;
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, 1,2,4-triazol-4-on-2-yl, tetrazol-5-yl, 2-methyl-tetrazol-5-yl, 1-methyl-tetrazol-5-yl, imidazol-1-yl, 1-methyl-imidazol-3-yl, 1-methyl-5-amino-imidazol-3-yl, 3-methylimidazol-2-on-1-yl, 1-methyl-pyrazol-3-yl, 1-methyl-pyrazol-5-yl, pyrrol-1-yl, thiazol-2-yl, isothiazolidin-2-yl-1,1-dioxide, pyrrolidin-2-on-1-yl, oxazol-2-yl, oxadiazol-2-yl, 2-amino-pyrimidin-4-yl, —(C═O)4-methylpiperazin-1-yl, —(C═O)N(CH₃)₂, —(C═O)NHCH₃, methoxy, ethoxy, difluoromethoxy, methyl, cyano; and
R^{6C} is selected from the group consisting of: cyano, fluoro, chloro, methyl, ethyl, methoxy, methyl, and trifluoromethyl.

In some embodiments, when R⁴ is

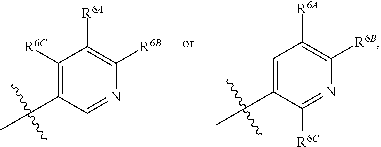

R^{6A} is selected from the group consisting of: cyano, chloro, and trifluoromethyl;
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, and 1,2,4-triazol-4-on-2-yl; and
R^{6C} is selected from the group consisting of: cyano, chloro, methyl, and trifluoromethyl.

In some embodiments, when R⁴ is

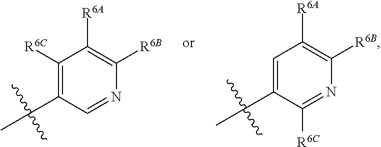

R^{6A} is chloro;
R^{6B} is selected from the group consisting of: 1,2,3-triazol-2-yl and 1,2,4-triazol-4-on-2-yl; and
R^{6C} is selected from the group consisting of: cyano, chloro, methyl, and trifluoromethyl.

In some embodiments, R⁴ is

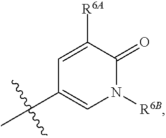

wherein the wavy line crosses the bond that connects to the —C(═O)NH— moiety of Formula (I).

In some embodiments, when R⁴ is

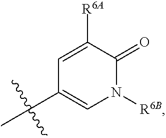

R^{6A} is selected from the group consisting of: cyano, halogen, C1-C3 alkyl, C1-C3 alkoxy, and C1-C3 haloalkyl;
R^{6B} is selected from the group consisting of: C1-C3 alkyl and C1-C3 haloalkyl.

In some embodiments, when R⁴ is

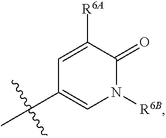

R^{6A} is selected from the group consisting of: cyano, fluoro, chloro, methyl, ethyl, methoxy, trifluoromethyl;
R^{6B} is selected from the group consisting of: methyl, ethyl, difluoromethyl, and trifluoromethyl;

In some embodiments, when R⁴ is

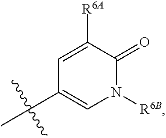

R^{6A} is chloro; and
R^{6B} is selected from the group consisting of: trifluoromethyl and difluoromethyl.

In some embodiments, R⁵ is hydrogen.

In some embodiments, R⁵ is halogen. For example, R⁵ is fluoro. For example, R⁵ is chloro. In some embodiments, R⁵ is cyano. In some embodiments, R⁵ is hydroxyl.

In some embodiments, R⁵ is C1-C3 alkoxy. In some embodiments, R⁵ is methoxy or ethoxy.

In some embodiments, R⁵ is C1-C3 haloalkoxy. In some embodiments, R⁵ is trifluoromethoxy, difluoromethoxy, or fluoromethoxy.

In some embodiments, R⁵ is C1-C3 haloalkyl. In some embodiments, R⁵ is trifluoromethyl or 2,2,2-trifluoroethyl.

In some embodiments, R⁵ is —NR^{C}R^{D}. In some embodiments, R^{C} and R^{D} are independently hydrogen or C1-C3 alkyl. In certain embodiments, one of R^{C} and R^{D} is hydrogen and the other of R^{C} and R^{D} is C1-C3 alkyl. In some embodiments, one of R^{C} and R^{D} is hydrogen and the other of R^{C} and R^{D} is methyl. In some embodiments, one of R^{C} and R^{D} is hydrogen and the other of R^{C} and R^{D} is ethyl. In certain embodiments, R^{C} and R^{D} are both hydrogens. In certain embodiments, R^{C} and R^{D} are both C1-C3 alkyl. In some embodiments, R^{C} and R^{D} are both methyl. In some embodiments, one of R^{C} and R^{D} is methyl and the other of R^{C} and R^{D} is ethyl. In some embodiments, R^{C} and R^{D} are both ethyl.

In some embodiments, R^{C} and R^{D} together with the nitrogen atom to which they are attached come together to form a 4-6 membered heterocyclyl. In certain embodiments, R^{C} and R^{D} together with the nitrogen atom to which they are attached come together to form a 4 membered heterocyclyl. In some embodiments, R^{C} and R^{D} together with the nitrogen atom to which they are attached come together to form a 5 membered heterocyclyl. In some embodiments, R^{C} and R^{D} together with the nitrogen atom to which they are attached come together to form a 6 membered heterocyclyl.

In some embodiments, R⁵ is C1-C3 alkyl. In some embodiments, R⁵ is methyl or ethyl.

In some embodiments,
X is N;
Y is C;
Z is N;
R¹ is halogen;
R² is hydrogen;
R^{2A} is hydrogen;
m is 2 and R³ is independently unsubstituted C1-C3 alkyl or C1-C3 haloalkoxy;
n is 1; and
R⁴ is 5-6 membered heteroaryl optionally substituted with 1-2 substituents independently selected from C1-C3 haloalkyl and 5-6 membered heteroaryl optionally substituted with 1-3 independently selected R^{X}.

In some embodiments, R¹ is chloro or fluoro.

In some embodiments, R² is hydrogen.

In some embodiments, R^{2A} is hydrogen.

In some embodiments, each R³ is geminal. In some embodiments, one R³ is unsubstituted C1-C3 alkyl and the other R³ is C1-C3 haloalkoxy. In some embodiments, one R³ is methyl and the other R³ is trifluoromethyl.

In some embodiments, R⁴ is unsubstituted 6 membered heteroaryl. In some embodiments, R⁴ is unsubstituted 1,2,3-triazolyl.

In some embodiments, the compound of Formula (I) is a compound of Formula (II):

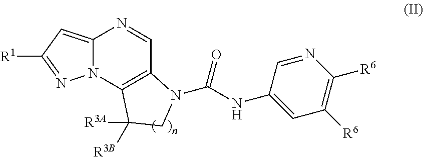

wherein:
n is 1 or 2;
R¹ is hydrogen, halogen, cyano, hydroxyl, C1-C3 alkoxy, C1-C3 haloalkoxy, C1-C3 haloalkyl, —NR^{A}R^{B}, or C1-C3 alkyl optionally substituted with 1-3 substituents independently selected from hydroxyl and C1-C3 alkoxy;
R^{3A} is halogen, hydroxyl, cyano, C3-C6 cycloalkyl, —NR^{A}R^{B}, 5-6 membered heteroaryl optionally substituted with C1-C3 alkyl; C1-C3 alkoxy, C1-C3 haloalkoxy, C1-C3 haloalkyl, or C1-C3 alkyl optionally substituted with a C1-C3 alkoxy or cyano;
R^{3B} is halogen, hydroxyl, cyano, C3-C6 cycloalkyl, —NR^{A}R^{B}, 5-6 membered heteroaryl optionally substituted with C1-C3 alkyl; C1-C3 alkoxy, C1-C3 haloalkoxy, C1-C3 haloalkyl, or C1-C3 alkyl optionally substituted with a C1-C3 alkoxy or cyano;
or R^{3A} and R^{3B} together with the carbon atom to which they are attached come together to form an oxo group or a C3-C8 cycloalkyl;
each R⁶ is independently selected from halogen; cyano; hydroxyl; —CO₂H; —N═(S═O)(C1-C3 alkyl)₂, —S(═O)ₚ(C1-C3 alkyl), —NR^{E}R^{F}; —(C═O)NR^{E}R^{F}; C1-C3 alkoxy optionally substituted with amino, hydroxyl, or —(C═O)NR^{E}R^{F}; C1-C3 haloalkyl; C1-C3 haloalkoxy; 5-6 membered heteroaryl optionally substituted with 1-3 independently selected R^{X}; C1-C3 alkyl optionally substituted with 1-2 substituents independently selected from hydroxyl, —NR^{E}R^{F}, C1-C3 alkoxy, and C3-C6 cycloalkyl; C3-C6 cycloalkyl optionally substituted with hydroxyl; and -(Q)_{q}-3-8 membered heterocyclyl optionally substituted with 1-3 independently selected C1-C3 alkyl;
p is 1 or 2;
Q is —O— or —NH—;
q is 0 or 1;
each R^{X} is independently selected from halogen, cyano, hydroxyl, amino, C1-C3 alkoxy, C1-C3 haloalkoxy, C1-C3 haloalkyl, or C1-C6 alkyl optionally substituted with 1-3 substituents independently selected from hydroxyl, C1-C3 alkoxy, and —NR^{G}R^{H}; and
R^{A} and R^{B} are independently hydrogen, C1-C3 alkyl, or R^{A} and R^{B}, together with the nitrogen atom to which they are attached come together to form a 4-6 membered heterocyclyl; and
R^{E}, R^{F}, R^{G}, and R^{H} are independently hydrogen, C1-C3 alkyl, or C3-C6 cycloalkyl, or R^{E} and R^{F}, or R^{G} and R^{H}, together with the nitrogen atom to which they are attached come together to form a 4-6 membered heterocyclyl optionally substituted with C1-C3 alkyl or C1-C3 alkoxy.

In some embodiments of Formula (II):
n is 1;
R¹ is hydrogen, halogen, or cyano;
one of R^{3A} and R^{3B} is halogen, hydroxyl, cyano, C3-C6 cycloalkyl, C1-C3 alkoxy, C1-C3 haloalkoxy, C1-C3 haloalkyl, or C1-C3 alkyl optionally substituted with a C1-C3 alkoxy or cyano; and the other of R^{3A} and R^{3B} is C1-C3 alkoxy, C1-C3 haloalkoxy, C1-C3 haloalkyl, or C1-C3 alkyl optionally substituted with a C1-C3 alkoxy or cyano;
each R⁶ is independently selected from halogen; cyano; hydroxyl; —CO₂H; —N═(S═O)(C1-C3 alkyl)₂, —S(═O)ₚ(C1-C3 alkyl), —NR^{E}R^{F}; —(C═O)NR^{E}R^{F}; C1-C3 alkoxy optionally substituted with amino, hydroxyl, or —(C═O)NR^{E}R^{F}; C1-C3 haloalkyl; C1-C3 haloalkoxy; 5-6 membered heteroaryl optionally substituted with 1-3 independently selected R^{X}; C1-C3 alkyl optionally substituted with 1-2 substituents independently selected from hydroxyl, —NR^{E}R^{F}, C1-C3 alkoxy, and C3-C6 cycloalkyl; and C3-C6 cycloalkyl optionally substituted with hydroxyl;
p is 1 or 2;
each R^{X} is independently selected from halogen, cyano, hydroxyl, amino, C1-C3 alkoxy, C1-C3 haloalkoxy, C1-C3 haloalkyl, or C1-C6 alkyl optionally substituted with 1-3 substituents independently selected from hydroxyl, C1-C3 alkoxy, and —NR^{G}R^{H}; and
R^{E}, R^{F}, R^{G}, and R^{H} are independently hydrogen, C1-C3 alkyl, or C3-C6 cycloalkyl, or R^{E} and R^{F}, or R^{G} and R^{H}, together with the nitrogen atom to which they are attached come together to form a 4-6 membered heterocyclyl optionally substituted with C1-C3 alkyl or C1-C3 alkoxy.

In some embodiments of Formula (II):
n is 2;
R¹ is hydrogen, halogen, or cyano;
one of R^{3A} and R^{3B} is halogen, hydroxyl, cyano, C3-C6 cycloalkyl, C1-C3 alkoxy, C1-C3 haloalkoxy, C1-C3 haloalkyl, or C1-C3 alkyl optionally substituted with a C1-C3 alkoxy or cyano; and the other of R^{3A} and R^{3B} is C1-C3 alkoxy, C1-C3 haloalkoxy, C1-C3 haloalkyl, or C1-C3 alkyl optionally substituted with a C1-C3 alkoxy or cyano;
each R⁶ is independently selected from halogen; cyano; hydroxyl; —CO₂H; —N═(S═O)(C1-C3 alkyl)₂, —S(═O)ₚ(C1-C3 alkyl), —NR^{E}R^{F}; —(C═O)NR^{E}R^{F}; C1-C3 alkoxy optionally substituted with amino, hydroxyl, or —(C═O)NR^{E}R^{F}; C1-C3 haloalkyl; C1-C3 haloalkoxy; 5-6 membered heteroaryl optionally substituted with 1-3 independently selected R^{X}; C1-C3 alkyl optionally substituted with 1-2 substituents independently selected from hydroxyl, —NR^{E}R^{F}, C1-C3 alkoxy, and C3-C6 cycloalkyl; and C3-C6 cycloalkyl optionally substituted with hydroxyl;
p is 1 or 2;
each R^{X} is independently selected from halogen, cyano, hydroxyl, amino, C1-C3 alkoxy, C1-C3 haloalkoxy, C1-C3 haloalkyl, or C1-C6 alkyl optionally substituted with 1-3 substituents independently selected from hydroxyl, C1-C3 alkoxy, and —NR^{G}R^{H}; and
R^{E}, R^{F}, R^{G}, and R^{H} are independently hydrogen, C1-C3 alkyl, or C3-C6 cycloalkyl, or R^{E} and R^{F}, or R^{G} and R^{H}, together with the nitrogen atom to which they are attached come together to form a 4-6 membered heterocyclyl optionally substituted with C1-C3 alkyl or C1-C3 alkoxy.

In some embodiments, the compound is a compound selected from Table 1, or a pharmaceutically acceptable salt thereof. Unless otherwise indicated, (1) the stereochemical configuration of each stereocenter shown with dash and wedge bond notation and/or an adjacent CIP configuration is assumed to be relative; and (2) any stereocenter whose valency is filled with bonds that are not depicted using dash and wedge is a mixture of stereochemical configurations at that stereocenter. For example, compounds 3 and 4 are enantiomers, but it is not yet known which is the (R) enantiomer and which is the (S) enantiomer. In another example, compounds 33 and 34 are diastereomers in which the absolute configuration of the stereocenter attached to the trifluoromethyl is known, but the stereocenter in the tetrahydrofuryl group is relative (i.e., the tetrahydrofuryl stereocenter in one of compounds 33 and 34 has the (R) configuration, and the tetrahydrofuryl stereocenter in the other of compounds 33 and 34 has the (S) configuration).

TABLE 1

| Compound | |
|---|---|
| Number | Structure |
| 1 | 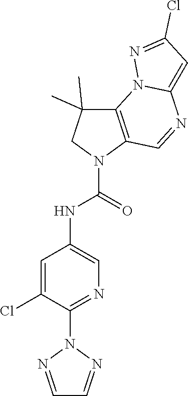 [SMILES: CC1(C)CN(C(=O)Nc2cnc(-n3nccn3)c(Cl)c2)c2cnc3cc(Cl)nn3c21] |
| 2 | 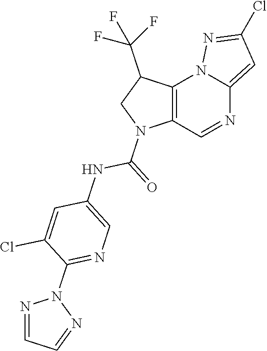 [SMILES: O=C(Nc1cnc(-n2nccn2)c(Cl)c1)N1CC(C(F)(F)F)c2c1cnc1cc(Cl)nn21] |
| 3 | 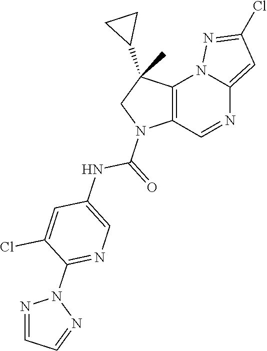 [SMILES: C[C@]1(C2CC2)CN(C(=O)Nc2cnc(-n3nccn3)c(Cl)c2)c2cnc3cc(Cl)nn3c21] |
| 4 | 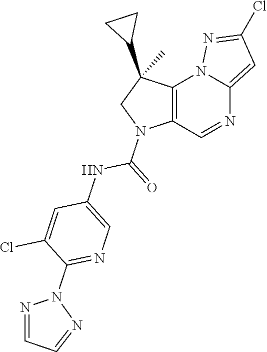 |
| 5 | 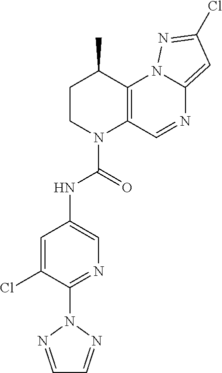 [SMILES: C[C@@H]1CCN(C(=O)Nc2cnc(-n3nccn3)c(Cl)c2)c2cnc3cc(Cl)nn3c21] |
| 6 | 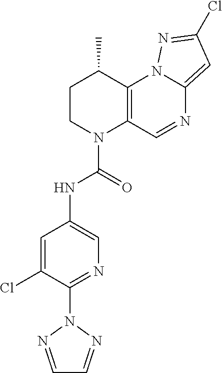 [SMILES: C[C@H]1CCN(C(=O)Nc2cnc(-n3nccn3)c(Cl)c2)c2cnc3cc(Cl)nn3c21] |
| 7 | 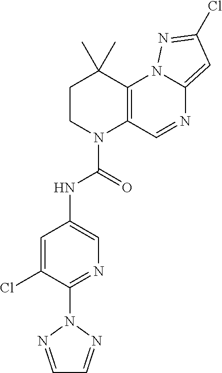 [SMILES: CC1(C)CCN(C(=O)Nc2cnc(-n3nccn3)c(Cl)c2)c2cnc3cc(Cl)nn3c21] |
| 8 | 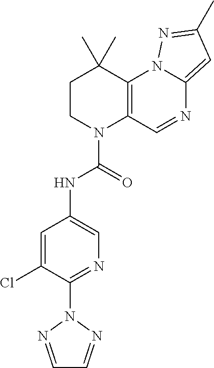 [SMILES: Cc1cc2ncc3c(n2n1)C(C)(C)CCN3C(=O)Nc1cnc(-n2nccn2)c(Cl)c1] |
| 9 | 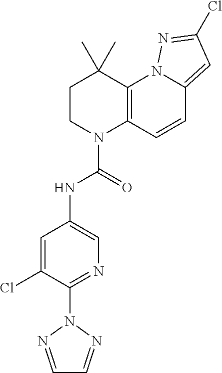 [SMILES: CC1(C)CCN(C(=O)Nc2cnc(-n3nccn3)c(Cl)c2)c2ccc3cc(Cl)nn3c21] |
| 10 | 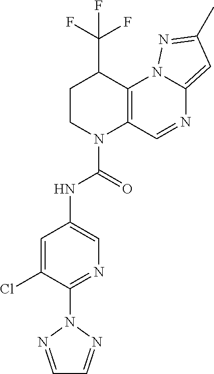 [SMILES: Cc1cc2ncc3c(n2n1)C(C(F)(F)F)CCN3C(=O)Nc1cnc(-n2nccn2)c(Cl)c1] |
| 11 | 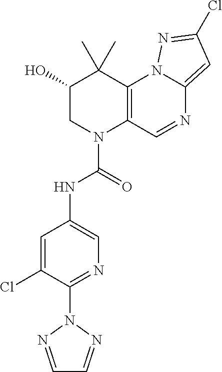 [SMILES: CC1(C)c2c(cnc3cc(Cl)nn23)N(C(=O)Nc2cnc(-n3nccn3)c(Cl)c2)C[C@@H]1O] |
| 12 | 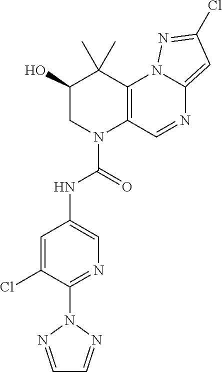 [SMILES: CC1(C)c2c(cnc3cc(Cl)nn23)N(C(=O)Nc2cnc(-n3nccn3)c(Cl)c2)C[C@H]1O] |
| 13 | 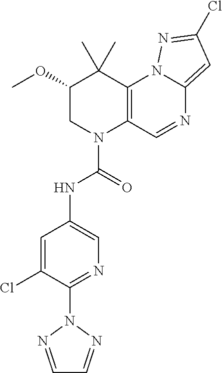 [SMILES: CO[C@H]1CN(C(=O)Nc2cnc(-n3nccn3)c(Cl)c2)c2cnc3cc(Cl)nn3c2C1(C)C] |
| 14 | 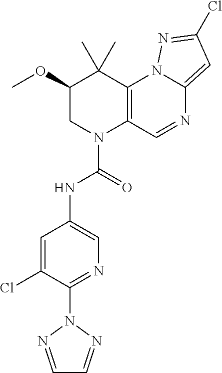 [SMILES: CO[C@@H]1CN(C(=O)Nc2cnc(-n3nccn3)c(Cl)c2)c2cnc3cc(Cl)nn3c2C1(C)C] |
| 15 | 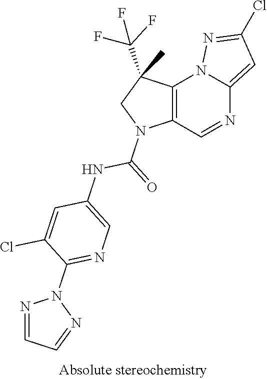 [SMILES: C[C@]1(C(F)(F)F)CN(C(=O)Nc2cnc(-n3nccn3)c(Cl)c2)c2cnc3cc(Cl)nn3c21] |
| 16 | 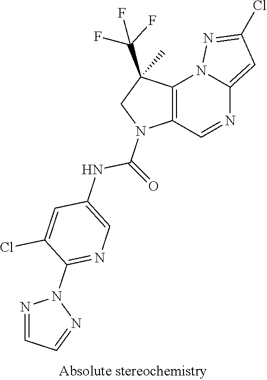 [SMILES: C[C@@]1(C(F)(F)F)CN(C(=O)Nc2cnc(-n3nccn3)c(Cl)c2)c2cnc3cc(Cl)nn3c21] |
| 17 | 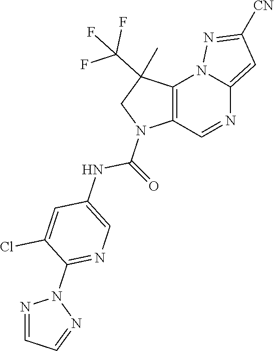 [SMILES: CC1(C(F)(F)F)CN(C(=O)Nc2cnc(-n3nccn3)c(Cl)c2)c2cnc3cc(C#N)nn3c21] |
| 18 | 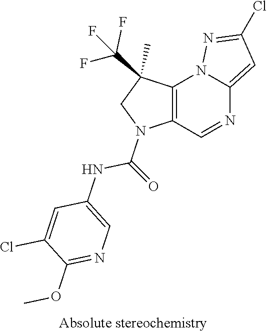 [SMILES: COc1ncc(NC(=O)N2C[C@@](C)(C(F)(F)F)c3c2cnc2cc(Cl)nn32)cc1Cl] |
| 19 | 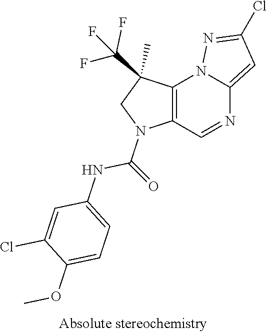 [SMILES: COc1ccc(NC(=O)N2C[C@@](C)(C(F)(F)F)c3c2cnc2cc(Cl)nn32)cc1Cl] |
| 20 | 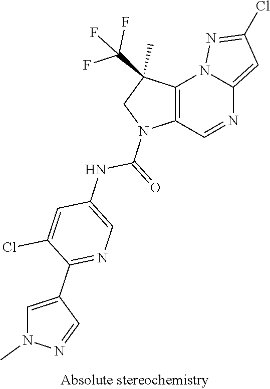 [SMILES: Cn1cc(-c2ncc(NC(=O)N3C[C@@](C)(C(F)(F)F)c4c3cnc3cc(Cl)nn43)cc2Cl)cn1] |
| 21 | 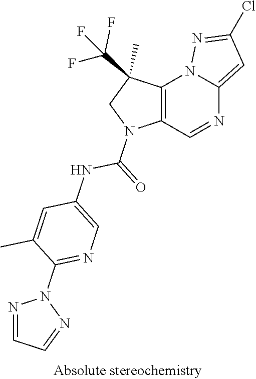 [SMILES: Cc1cc(NC(=O)N2C[C@@](C)(C(F)(F)F)c3c2cnc2cc(Cl)nn32)cnc1-n1nccn1] |
| 22 | 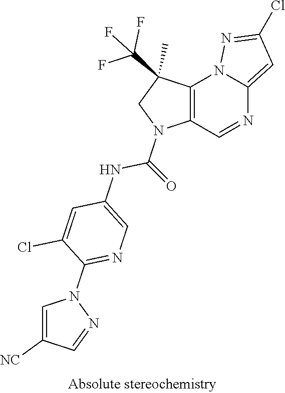 [SMILES: C[C@@]1(C(F)(F)F)CN(C(=O)Nc2cnc(-n3cc(C#N)cn3)c(Cl)c2)c2cnc3cc(Cl)nn3c21] |
| 23 | 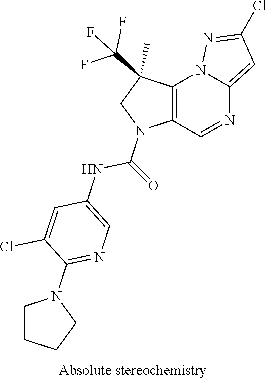 [SMILES: C[C@@]1(C(F)(F)F)CN(C(=O)Nc2cnc(N3CCCC3)c(Cl)c2)c2cnc3cc(Cl)nn3c21] |
| 24 | 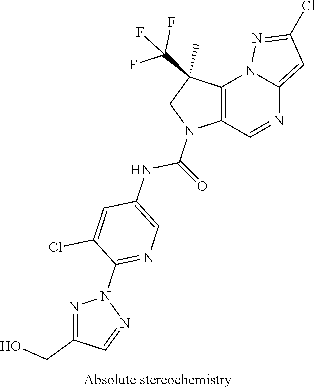 [SMILES: C[C@@]1(C(F)(F)F)CN(C(=O)Nc2cnc(-n3ncc(CO)n3)c(Cl)c2)c2cnc3cc(Cl)nn3c21] |
| 25 | 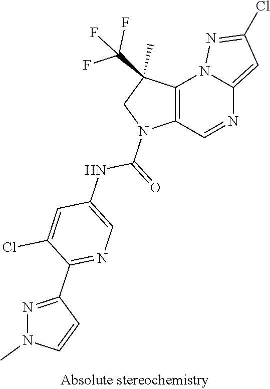 [SMILES: Cn1ccc(-c2ncc(NC(=O)N3C[C@@](C)(C(F)(F)F)c4c3cnc3cc(Cl)nn43)cc2Cl)n1] |
| 26 | 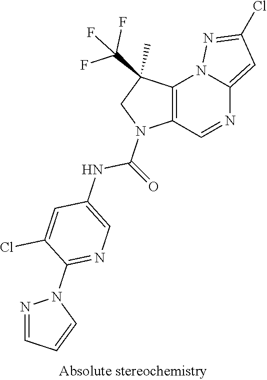 [SMILES: C[C@@]1(C(F)(F)F)CN(C(=O)Nc2cnc(-n3cccn3)c(Cl)c2)c2cnc3cc(Cl)nn3c21] |
| 27 | 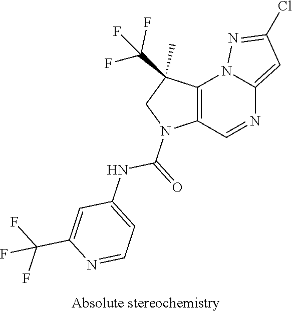 [SMILES: C[C@@]1(C(F)(F)F)CN(C(=O)Nc2ccnc(C(F)(F)F)c2)c2cnc3cc(Cl)nn3c21] |
| 28 | 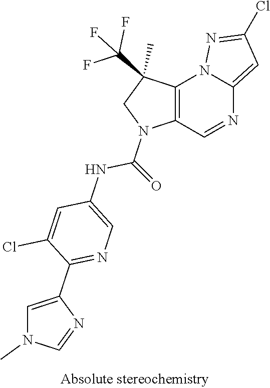 [SMILES: Cn1cnc(-c2ncc(NC(=O)N3C[C@@](C)(C(F)(F)F)c4c3cnc3cc(Cl)nn43)cc2Cl)c1] |
| 29 | 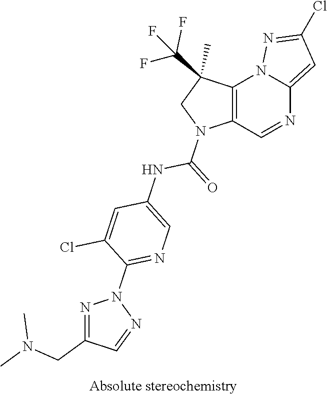 [SMILES: CN(C)Cc1cnn(-c2ncc(NC(=O)N3C[C@@](C)(C(F)(F)F)c4c3cnc3cc(Cl)nn43)cc2Cl)n1] |
| 30 | 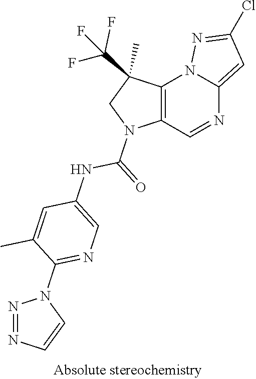 [SMILES: Cc1cc(NC(=O)N2C[C@@](C)(C(F)(F)F)c3c2cnc2cc(Cl)nn32)cnc1-n1ccnn1] |
| 31 | 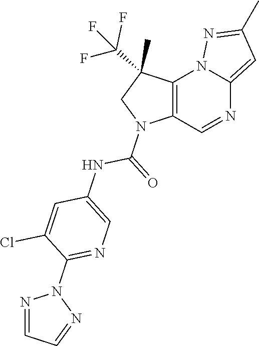 [SMILES: Cc1cc2ncc3c(n2n1)[C@@](C)(C(F)(F)F)CN3C(=O)Nc1cnc(-n2nccn2)c(Cl)c1] |
| 32 | 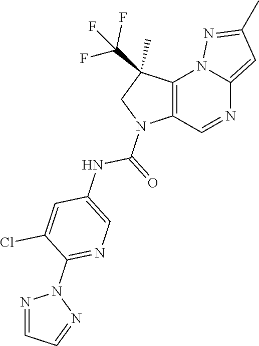 [SMILES: Cc1cc2ncc3c(n2n1)[C@](C)(C(F)(F)F)CN3C(=O)Nc1cnc(-n2nccn2)c(Cl)c1] |
| 33 | 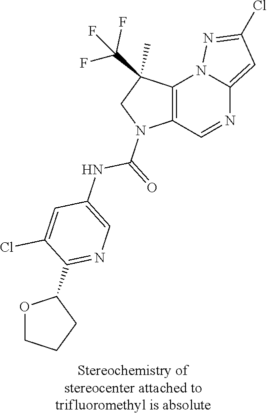 [SMILES: C[C@@]1(C(F)(F)F)CN(C(=O)Nc2cnc([C@@H]3CCCO3)c(Cl)c2)c2cnc3cc(Cl)nn3c21] |
| 34 | 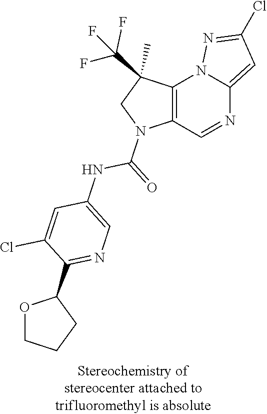 [SMILES: C[C@@]1(C(F)(F)F)CN(C(=O)Nc2cnc([C@H]3CCCO3)c(Cl)c2)c2cnc3cc(Cl)nn3c21] |
| 35 | 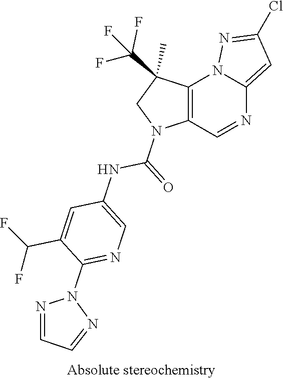 |
| 36 | 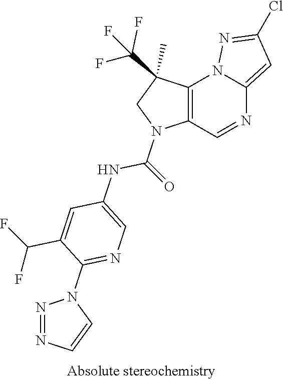 [SMILES: C[C@@]1(C(F)(F)F)CN(C(=O)Nc2cnc(-n3ccnn3)c(C(F)F)c2)c2cnc3cc(Cl)nn3c21] |
| 37 | 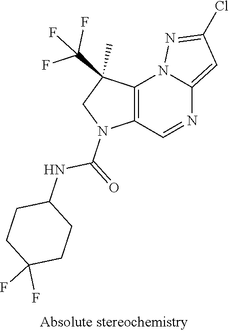 [SMILES: C[C@@]1(C(F)(F)F)CN(C(=O)NC2CCC(F)(F)CC2)c2cnc3cc(Cl)nn3c21] |
| 38 | 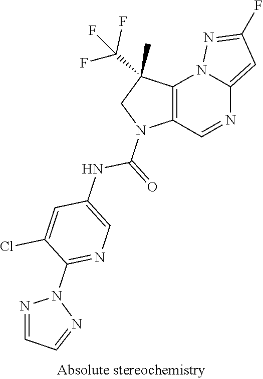 |
| 39 | 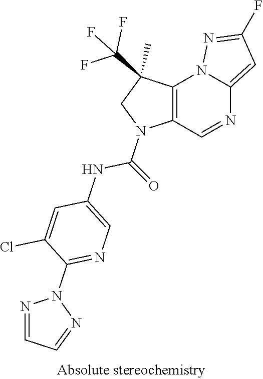 [SMILES: C[C@@]1(C(F)(F)F)CN(C(=O)Nc2cnc(-n3nccn3)c(Cl)c2)c2cnc3cc(F)nn3c21] |
| 40 | 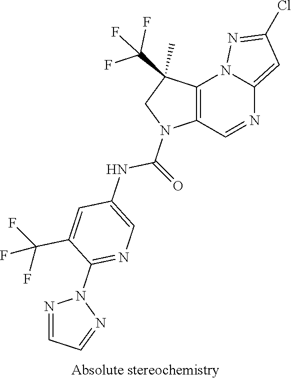 [SMILES: C[C@@]1(C(F)(F)F)CN(C(=O)Nc2cnc(-n3nccn3)c(C(F)(F)F)c2)c2cnc3cc(Cl)nn3c21] |
| 41 | 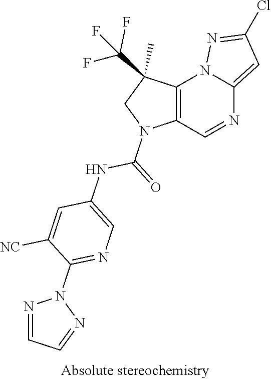 [SMILES: C[C@@]1(C(F)(F)F)CN(C(=O)Nc2cnc(-n3nccn3)c(C#N)c2)c2cnc3cc(Cl)nn3c21] |
| 42 | 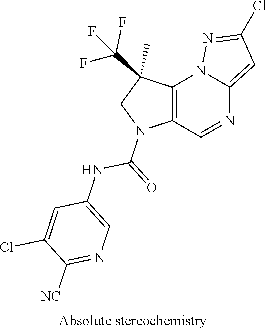 [SMILES: C[C@@]1(C(F)(F)F)CN(C(=O)Nc2cnc(C#N)c(Cl)c2)c2cnc3cc(Cl)nn3c21] |
| 43 | 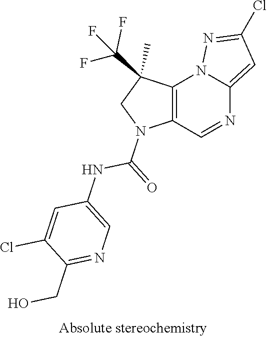 [SMILES: C[C@@]1(C(F)(F)F)CN(C(=O)Nc2cnc(CO)c(Cl)c2)c2cnc3cc(Cl)nn3c21] |
| 44 | 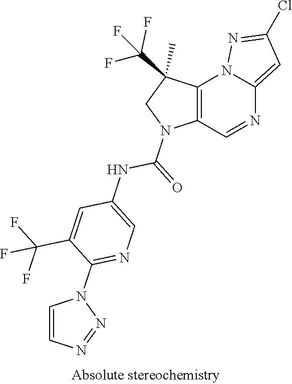 [SMILES: C[C@@]1(C(F)(F)F)CN(C(=O)Nc2cnc(-n3ccnn3)c(C(F)(F)F)c2)c2cnc3cc(Cl)nn3c21] |
| 45 | 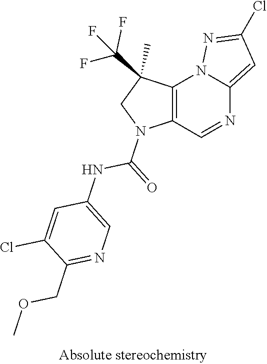 [SMILES: COCc1ncc(NC(=O)N2C[C@@](C)(C(F)(F)F)c3c2cnc2cc(Cl)nn32)cc1Cl] |
| 46 | 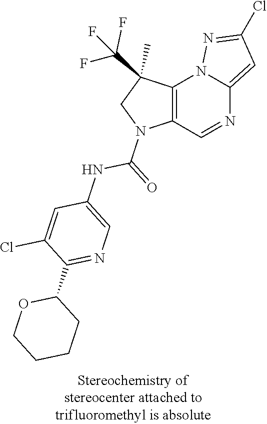 [SMILES: C[C@@]1(C(F)(F)F)CN(C(=O)Nc2cnc([C@@H]3CCCCO3)c(Cl)c2)c2cnc3cc(Cl)nn3c21] |
| 47 | 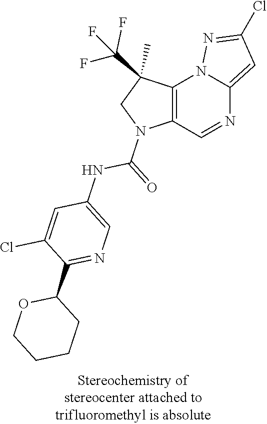 [SMILES: C[C@@]1(C(F)(F)F)CN(C(=O)Nc2cnc([C@H]3CCCCO3)c(Cl)c2)c2cnc3cc(Cl)nn3c21] |
| 48 | 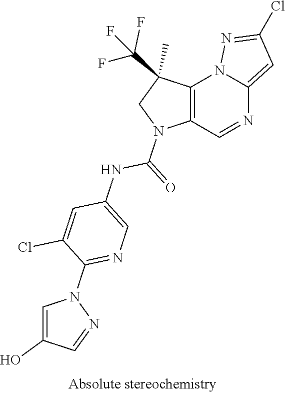 [SMILES: C[C@@]1(C(F)(F)F)CN(C(=O)Nc2cnc(-n3cc(O)cn3)c(Cl)c2)c2cnc3cc(Cl)nn3c21] |
| 49 | 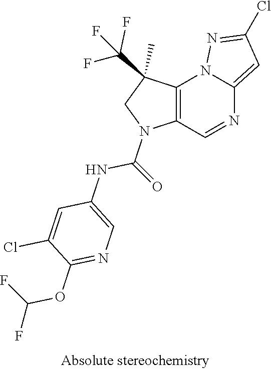 [SMILES: C[C@@]1(C(F)(F)F)CN(C(=O)Nc2cnc(OC(F)F)c(Cl)c2)c2cnc3cc(Cl)nn3c21] |
| 50 | 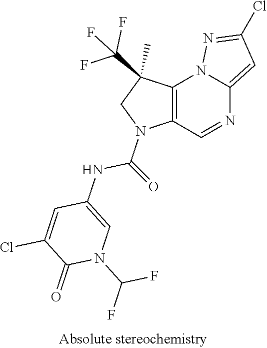 [SMILES: C[C@@]1(C(F)(F)F)CN(C(=O)Nc2cc(Cl)c(=O)n(C(F)F)c2)c2cnc3cc(Cl)nn3c21] |
| 51 | 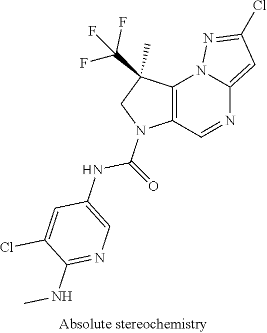 [SMILES: CNc1ncc(NC(=O)N2C[C@@](C)(C(F)(F)F)c3c2cnc2cc(Cl)nn32)cc1Cl] |
| 52 | 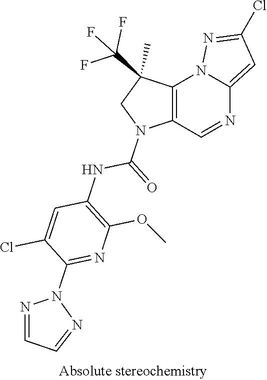 |
| 53 | 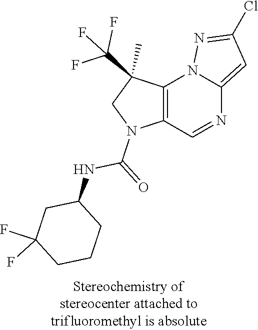 [SMILES: C[C@@]1(C(F)(F)F)CN(C(=O)N[C@H]2CCCC(F)(F)C2)c2cnc3cc(Cl)nn3c21] |
| 54 | 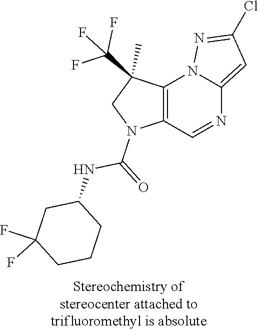 [SMILES: C[C@@]1(C(F)(F)F)CN(C(=O)N[C@@H]2CCCC(F)(F)C2)c2cnc3cc(Cl)nn3c21] |
| 55 | 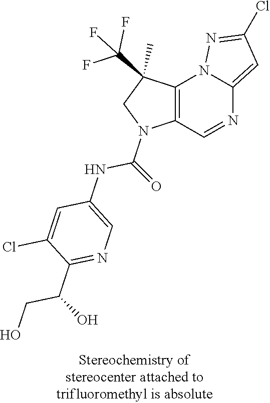 [SMILES: C[C@@]1(C(F)(F)F)CN(C(=O)Nc2cnc([C@H](O)CO)c(Cl)c2)c2cnc3cc(Cl)nn3c21] |
| 56 | 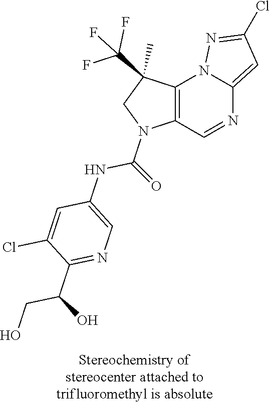 [SMILES: C[C@@]1(C(F)(F)F)CN(C(=O)Nc2cnc([C@@H](O)CO)c(Cl)c2)c2cnc3cc(Cl)nn3c21] |
| 57 | 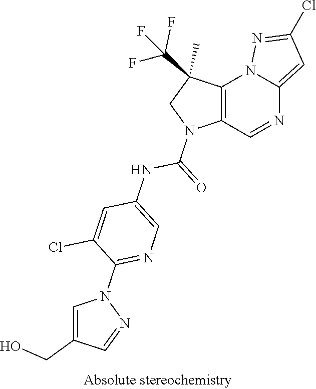 |
| 58 | 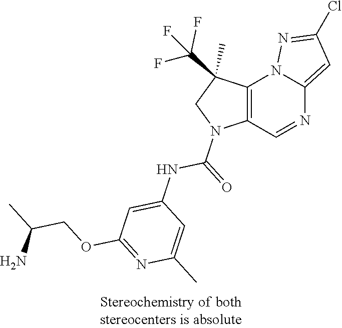 [SMILES: Cc1cc(NC(=O)N2C[C@@](C)(C(F)(F)F)c3c2cnc2cc(Cl)nn32)cc(OC[C@H](C)N)n1] |
| 59 | 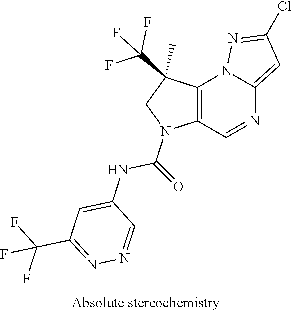 [SMILES: C[C@@]1(C(F)(F)F)CN(C(=O)Nc2cnnc(C(F)(F)F)c2)c2cnc3cc(Cl)nn3c21] |
| 60 | 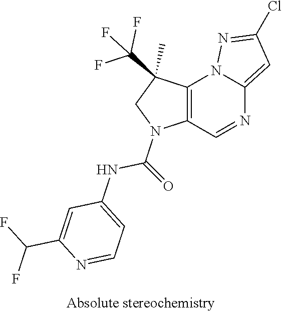 [SMILES: C[C@@]1(C(F)(F)F)CN(C(=O)Nc2ccnc(C(F)F)c2)c2cnc3cc(Cl)nn3c21] |
| 61 | 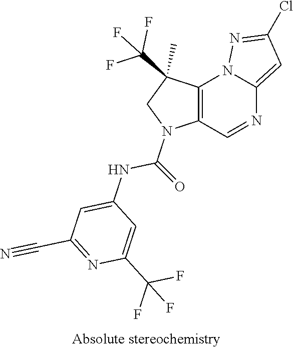 [SMILES: C[C@@]1(C(F)(F)F)CN(C(=O)Nc2cc(C#N)nc(C(F)(F)F)c2)c2cnc3cc(Cl)nn3c21] |
| 62 | 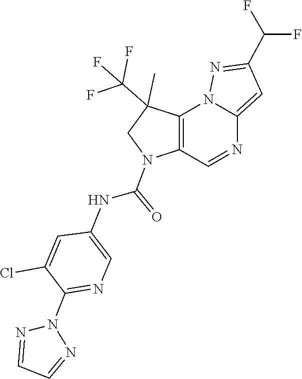 [SMILES: CC1(C(F)(F)F)CN(C(=O)Nc2cnc(-n3nccn3)c(Cl)c2)c2cnc3cc(C(F)F)nn3c21] |
| 63 | 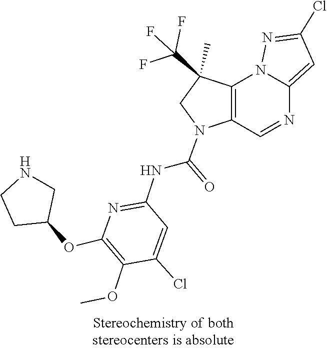 [SMILES: COc1c(Cl)cc(NC(=O)N2C[C@@](C)(C(F)(F)F)c3c2cnc2cc(Cl)nn32)nc1O[C@H]1CCNC1] |
| 64 | 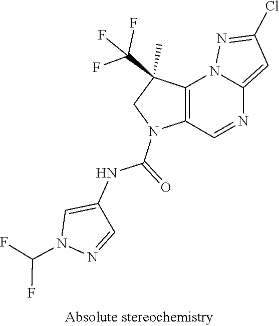 [SMILES: C[C@@]1(C(F)(F)F)CN(C(=O)Nc2cnn(C(F)F)c2)c2cnc3cc(Cl)nn3c21] |
| 65 | 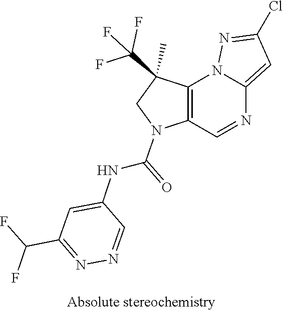 [SMILES: C[C@@]1(C(F)(F)F)CN(C(=O)Nc2cnnc(C(F)F)c2)c2cnc3cc(Cl)nn3c21] |
| 66 | 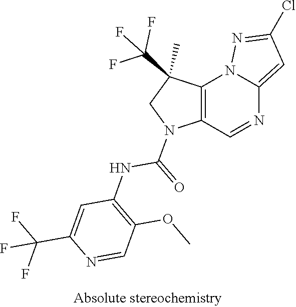 [SMILES: COc1cnc(C(F)(F)F)cc1NC(=O)N1C[C@@](C)(C(F)(F)F)c2c1cnc1cc(Cl)nn21] |
| 67 | 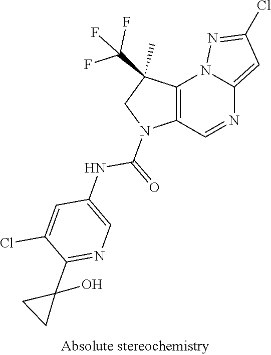 [SMILES: C[C@@]1(C(F)(F)F)CN(C(=O)Nc2cnc(C3(O)CC3)c(Cl)c2)c2cnc3cc(Cl)nn3c21] |
| 68 | 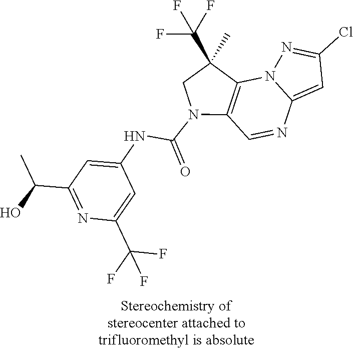 [SMILES: C[C@H](O)c1cc(NC(=O)N2C[C@@](C)(C(F)(F)F)c3c2cnc2cc(Cl)nn32)cc(C(F)(F)F)n1] |
| 69 | 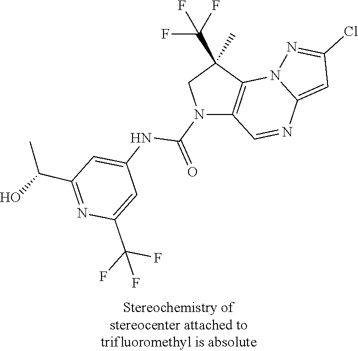 [SMILES: C[C@@H](O)c1cc(NC(=O)N2C[C@@](C)(C(F)(F)F)c3c2cnc2cc(Cl)nn32)cc(C(F)(F)F)n1] |
| 70 | 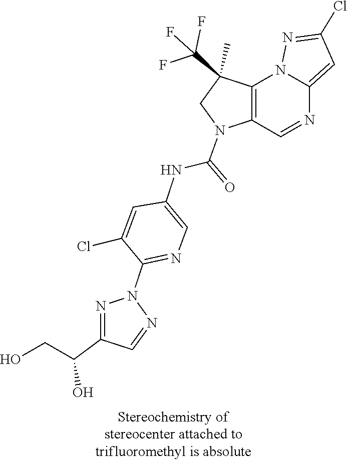 [SMILES: C[C@@]1(C(F)(F)F)CN(C(=O)Nc2cnc(-n3ncc([C@H](O)CO)n3)c(Cl)c2)c2cnc3cc(Cl)nn3c21] |
| 71 | 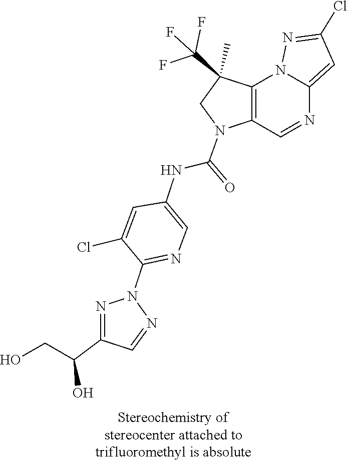 [SMILES: C[C@@]1(C(F)(F)F)CN(C(=O)Nc2cnc(-n3ncc([C@@H](O)CO)n3)c(Cl)c2)c2cnc3cc(Cl)nn3c21] |
| 72 | 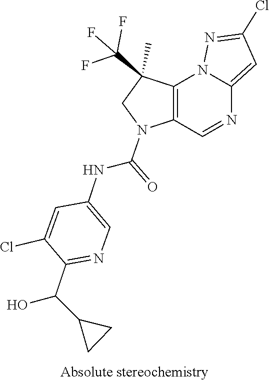 [SMILES: C[C@@]1(C(F)(F)F)CN(C(=O)Nc2cnc(C(O)C3CC3)c(Cl)c2)c2cnc3cc(Cl)nn3c21] |
| 73 | 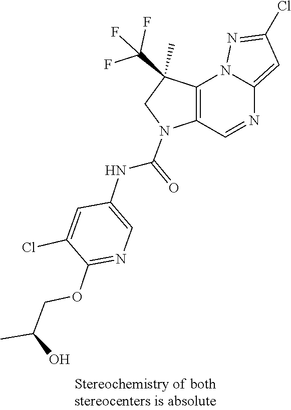 [SMILES: C[C@H](O)COc1ncc(NC(=O)N2C[C@@](C)(C(F)(F)F)c3c2cnc2cc(Cl)nn32)cc1Cl] |
| 74 | 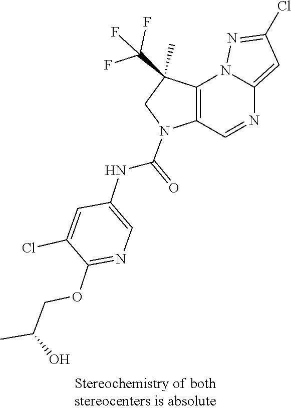 [SMILES: C[C@@H](O)COc1ncc(NC(=O)N2C[C@@](C)(C(F)(F)F)c3c2cnc2cc(Cl)nn32)cc1Cl] |
| 75 | 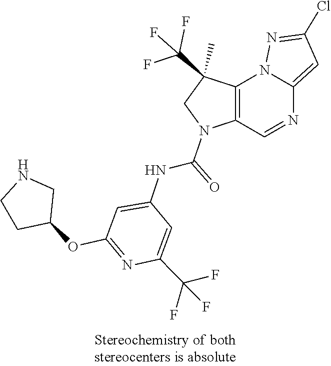 [SMILES: C[C@@]1(C(F)(F)F)CN(C(=O)Nc2cc(O[C@H]3CCNC3)nc(C(F)(F)F)c2)c2cnc3cc(Cl)nn3c21] |
| 76 | 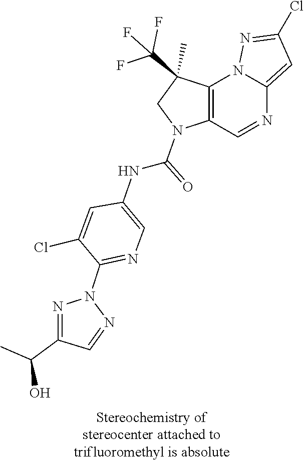 |
| 77 | 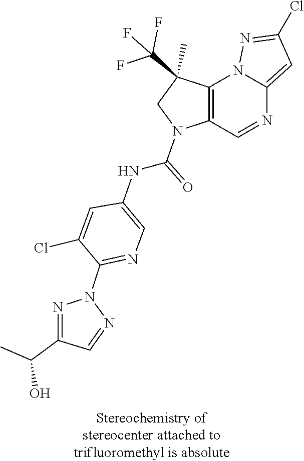 |
| 78 | 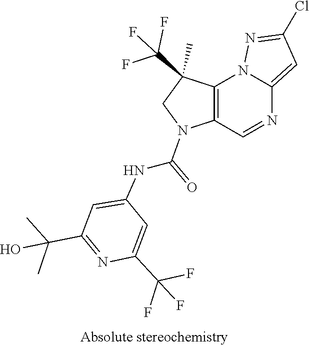 [SMILES: CC(C)(O)c1cc(NC(=O)N2C[C@@](C)(C(F)(F)F)c3c2cnc2cc(Cl)nn32)cc(C(F)(F)F)n1] |
| 79 | 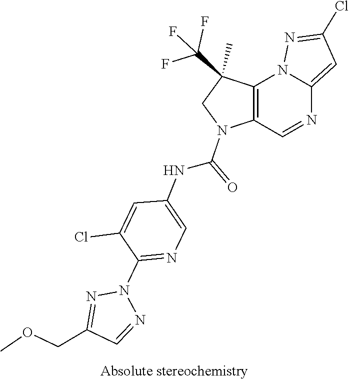 [SMILES: COCc1cnn(-c2ncc(NC(=O)N3C[C@@](C)(C(F)(F)F)c4c3cnc3cc(Cl)nn43)cc2Cl)n1] |
| 80 | 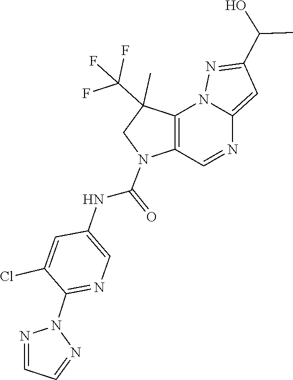 [SMILES: CC(O)c1cc2ncc3c(n2n1)C(C)(C(F)(F)F)CN3C(=O)Nc1cnc(-n2nccn2)c(Cl)c1] |
| 81 | 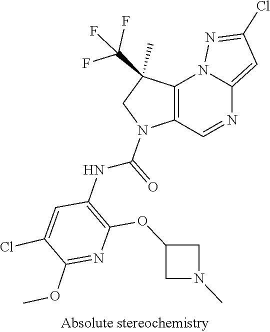 [SMILES: COc1nc(OC2CN(C)C2)c(NC(=O)N2C[C@@](C)(C(F)(F)F)c3c2cnc2cc(Cl)nn32)cc1Cl] |
| 82 | 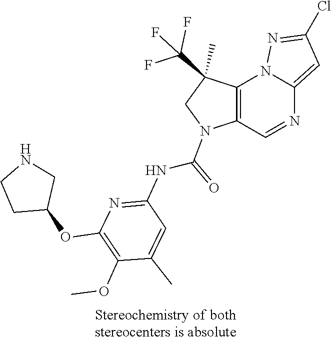 [SMILES: COc1c(C)cc(NC(=O)N2C[C@@](C)(C(F)(F)F)c3c2cnc2cc(Cl)nn32)nc1O[C@H]1CCNC1] |
| 83 | 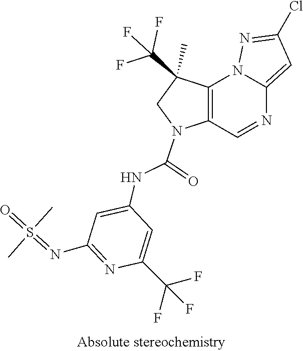 [SMILES: C[C@@]1(C(F)(F)F)CN(C(=O)Nc2cc(N=S(C)(C)=O)nc(C(F)(F)F)c2)c2cnc3cc(Cl)nn3c21] |
| 84 | 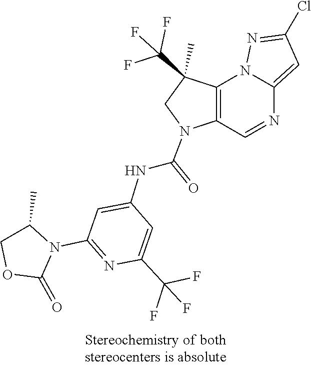 [SMILES: C[C@H]1COC(=O)N1c1cc(NC(=O)N2C[C@@](C)(C(F)(F)F)c3c2cnc2cc(Cl)nn32)cc(C(F)(F)F)n1] |
| 85 | 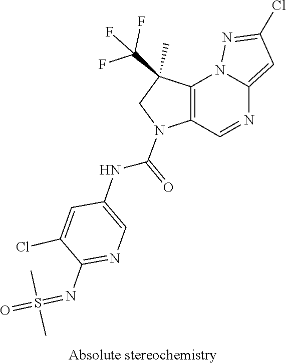 [SMILES: C[C@@]1(C(F)(F)F)CN(C(=O)Nc2cnc(N=S(C)(C)=O)c(Cl)c2)c2cnc3cc(Cl)nn3c21] |
| 86 | 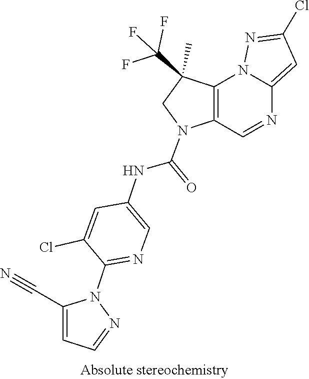 |
| 87 | 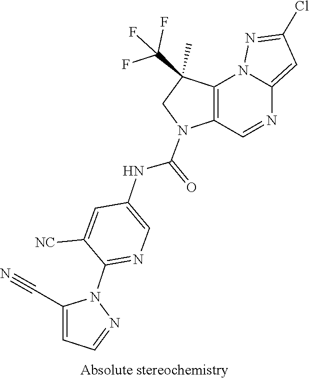 |
| 88 | 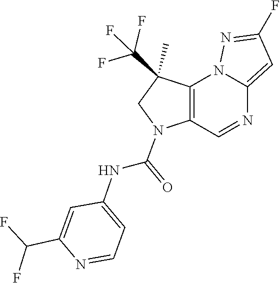 [SMILES: C[C@@]1(C(F)(F)F)CN(C(=O)Nc2ccnc(C(F)F)c2)c2cnc3cc(F)nn3c21] |
| 89 | 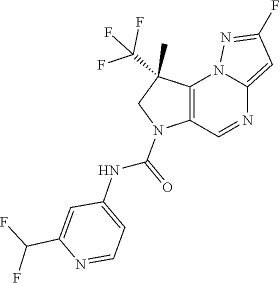 [SMILES: C[C@]1(C(F)(F)F)CN(C(=O)Nc2ccnc(C(F)F)c2)c2cnc3cc(F)nn3c21] |
| 90 | 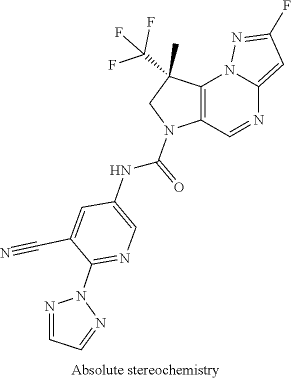 [SMILES: C[C@]1(C(F)(F)F)CN(C(=O)Nc2cnc(-n3nccn3)c(C#N)c2)c2cnc3cc(F)nn3c21] |
| 91 | 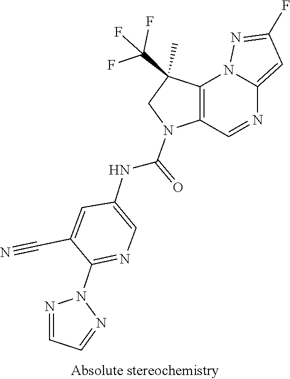 [SMILES: C[C@@]1(C(F)(F)F)CN(C(=O)Nc2cnc(-n3nccn3)c(C#N)c2)c2cnc3cc(F)nn3c21] |
| 92 | 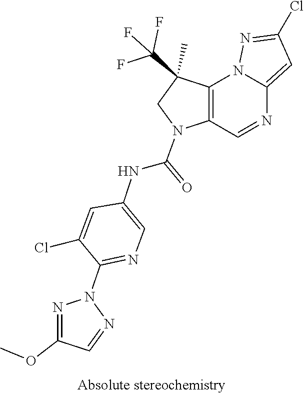 [SMILES: COc1cnn(-c2ncc(NC(=O)N3C[C@@](C)(C(F)(F)F)c4c3cnc3cc(Cl)nn43)cc2Cl)n1] |
| 93 | 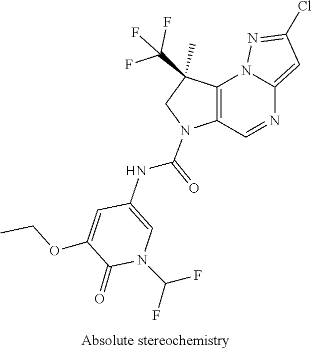 [SMILES: CCOc1cc(NC(=O)N2C[C@@](C)(C(F)(F)F)c3c2cnc2cc(Cl)nn32)cn(C(F)F)c1=O] |
| 94 | 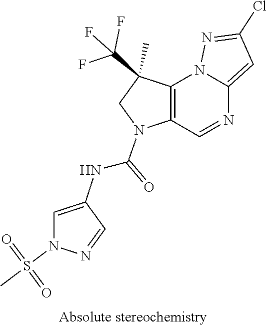 [SMILES: C[C@@]1(C(F)(F)F)CN(C(=O)Nc2cnn(S(C)(=O)=O)c2)c2cnc3cc(Cl)nn3c21] |
| 95 | 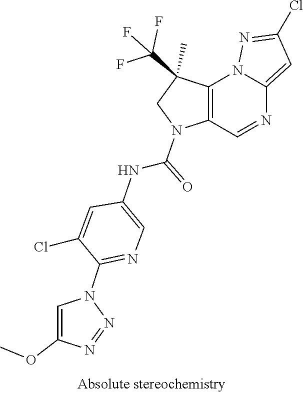 [SMILES: COc1cn(-c2ncc(NC(=O)N3C[C@@](C)(C(F)(F)F)c4c3cnc3cc(Cl)nn43)cc2Cl)nn1] |
| 96 | 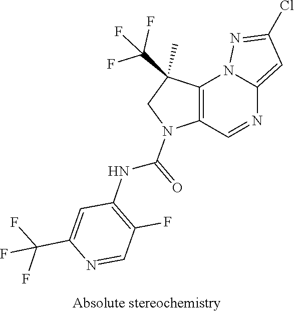 [SMILES: C[C@@]1(C(F)(F)F)CN(C(=O)Nc2cc(C(F)(F)F)ncc2F)c2cnc3cc(Cl)nn3c21] |
| 97 | 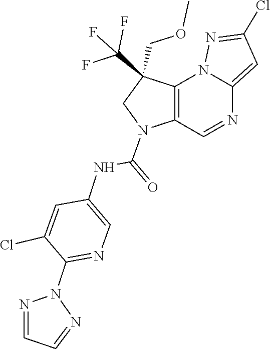 |
| 98 | 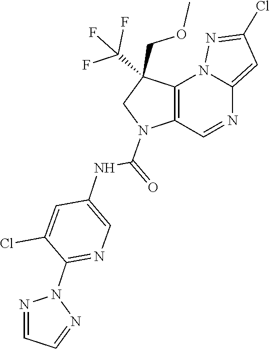 [SMILES: COC[C@]1(C(F)(F)F)CN(C(=O)Nc2cnc(-n3nccn3)c(Cl)c2)c2cnc3cc(Cl)nn3c21] |
| 99 | 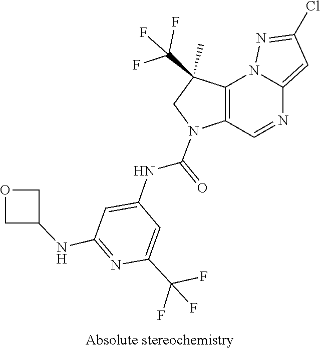 [SMILES: C[C@@]1(C(F)(F)F)CN(C(=O)Nc2cc(NC3COC3)nc(C(F)(F)F)c2)c2cnc3cc(Cl)nn3c21] |
| 100 | 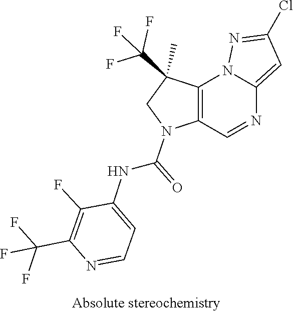 [SMILES: C[C@@]1(C(F)(F)F)CN(C(=O)Nc2ccnc(C(F)(F)F)c2F)c2cnc3cc(Cl)nn3c21] |
| 101 | 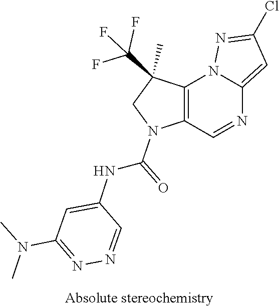 [SMILES: CN(C)c1cc(NC(=O)N2C[C@@](C)(C(F)(F)F)c3c2cnc2cc(Cl)nn32)cnn1] |
| 102 | 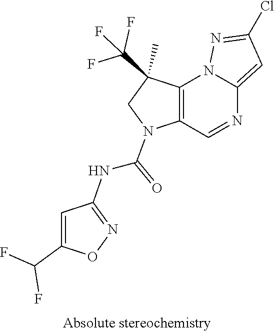 [SMILES: C[C@@]1(C(F)(F)F)CN(C(=O)Nc2cc(C(F)F)on2)c2cnc3cc(Cl)nn3c21] |
| 103 | 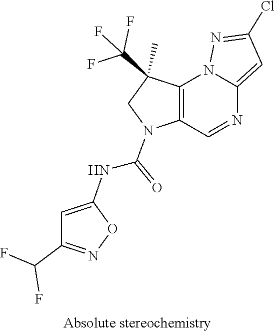 [SMILES: C[C@@]1(C(F)(F)F)CN(C(=O)Nc2cc(C(F)F)no2)c2cnc3cc(Cl)nn3c21] |
| 104 | 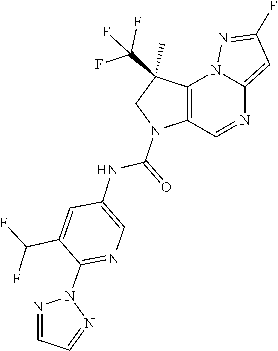 [SMILES: C[C@@]1(C(F)(F)F)CN(C(=O)Nc2cnc(-n3nccn3)c(C(F)F)c2)c2cnc3cc(F)nn3c21] |
| 105 | 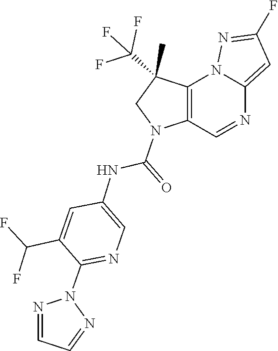 [SMILES: C[C@]1(C(F)(F)F)CN(C(=O)Nc2cnc(-n3nccn3)c(C(F)F)c2)c2cnc3cc(F)nn3c21] |
| 106 | 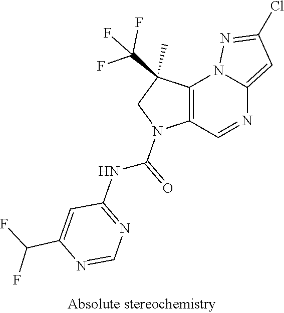 [SMILES: C[C@@]1(C(F)(F)F)CN(C(=O)Nc2cc(C(F)F)ncn2)c2cnc3cc(Cl)nn3c21] |
| 107 | 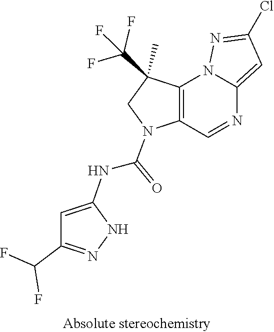 [SMILES: C[C@@]1(C(F)(F)F)CN(C(=O)Nc2cc(C(F)F)n[nH]2)c2cnc3cc(Cl)nn3c21] |
| 108 | 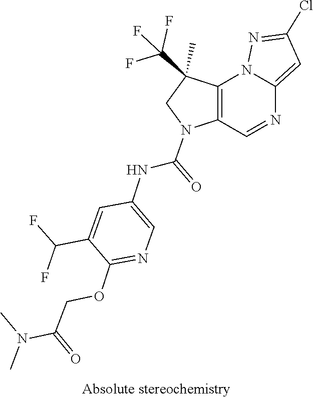 [SMILES: CN(C)C(=O)COc1ncc(NC(=O)N2C[C@@](C)(C(F)(F)F)c3c2cnc2cc(Cl)nn32)cc1C(F)F] |
| 109 | 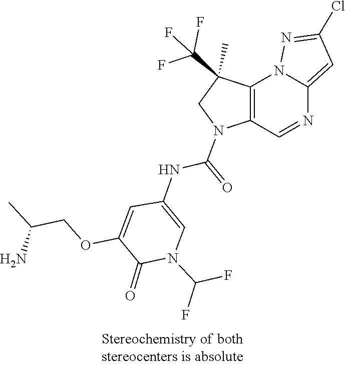 [SMILES: C[C@@H](N)COc1cc(NC(=O)N2C[C@@](C)(C(F)(F)F)c3c2cnc2cc(Cl)nn32)cn(C(F)F)c1=O] |
| 110 | 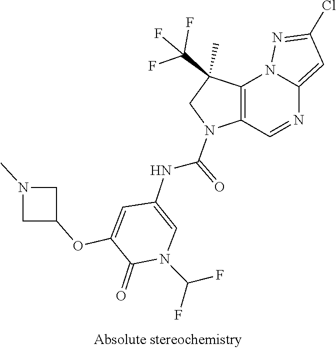 [SMILES: CN1CC(Oc2cc(NC(=O)N3C[C@@](C)(C(F)(F)F)c4c3cnc3cc(Cl)nn43)cn(C(F)F)c2=O)C1] |
| 111 | 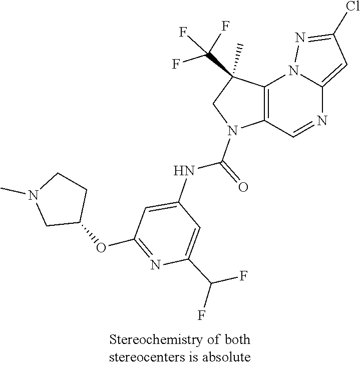 [SMILES: CN1CC[C@H](Oc2cc(NC(=O)N3C[C@@](C)(C(F)(F)F)c4c3cnc3cc(Cl)nn43)cc(C(F)F)n2)C1] |
| 112 | 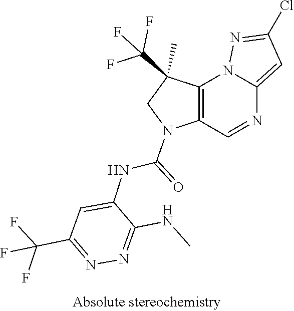 [SMILES: CNc1nnc(C(F)(F)F)cc1NC(=O)N1C[C@@](C)(C(F)(F)F)c2c1cnc1cc(Cl)nn21] |
| 113 | 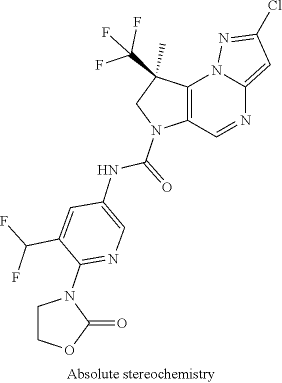 [SMILES: C[C@@]1(C(F)(F)F)CN(C(=O)Nc2cnc(N3CCOC3=O)c(C(F)F)c2)c2cnc3cc(Cl)nn3c21] |
| 114 | 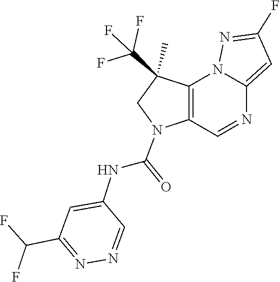 [SMILES: C[C@@]1(C(F)(F)F)CN(C(=O)Nc2cnnc(C(F)F)c2)c2cnc3cc(F)nn3c21] |
| 115 | 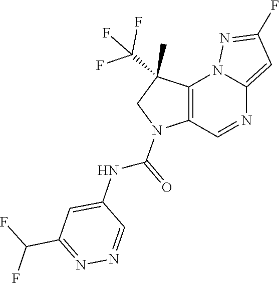 [SMILES: C[C@]1(C(F)(F)F)CN(C(=O)Nc2cnnc(C(F)F)c2)c2cnc3cc(F)nn3c21] |
| 116 | 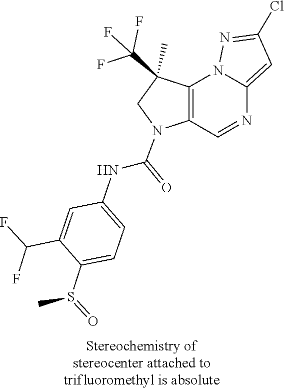 [SMILES: C[S@](=O)c1ccc(NC(=O)N2C[C@@](C)(C(F)(F)F)c3c2cnc2cc(Cl)nn32)cc1C(F)F] |
| 117 | 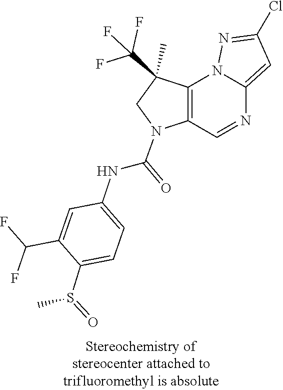 [SMILES: C[S@@](=O)c1ccc(NC(=O)N2C[C@@](C)(C(F)(F)F)c3c2cnc2cc(Cl)nn32)cc1C(F)F] |
| 118 | 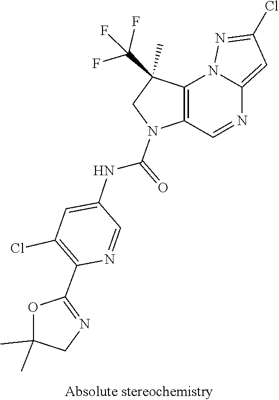 [SMILES: CC1(C)CN=C(c2ncc(NC(=O)N3C[C@@](C)(C(F)(F)F)c4c3cnc3cc(Cl)nn43)cc2Cl)O1] |
| 119 | 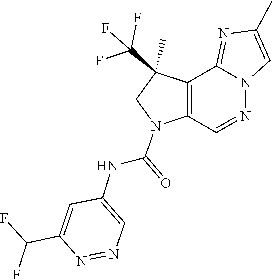 [SMILES: Cc1cn2ncc3c(c2n1)[C@](C)(C(F)(F)F)CN3C(=O)Nc1cnnc(C(F)F)c1] |
| 120 | 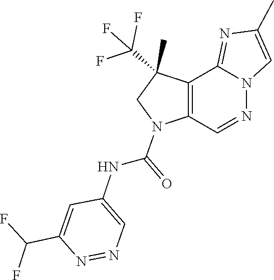 [SMILES: Cc1cn2ncc3c(c2n1)[C@@](C)(C(F)(F)F)CN3C(=O)Nc1cnnc(C(F)F)c1] |
| 121 | 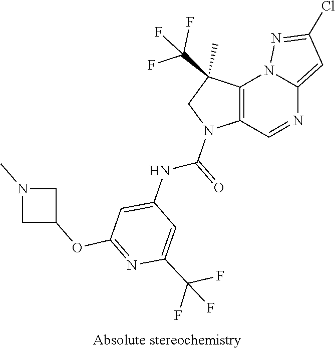 [SMILES: CN1CC(Oc2cc(NC(=O)N3C[C@@](C)(C(F)(F)F)c4c3cnc3cc(Cl)nn43)cc(C(F)(F)F)n2)C1] |
| 122 | 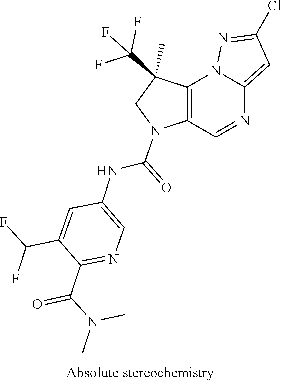 [SMILES: CN(C)C(=O)c1ncc(NC(=O)N2C[C@@](C)(C(F)(F)F)c3c2cnc2cc(Cl)nn32)cc1C(F)F] |
| 123 | 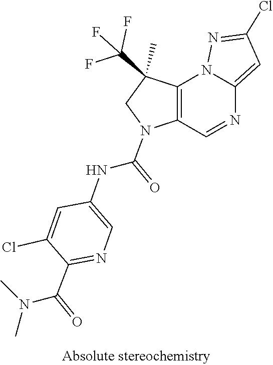 [SMILES: CN(C)C(=O)c1ncc(NC(=O)N2C[C@@](C)(C(F)(F)F)c3c2cnc2cc(Cl)nn32)cc1Cl] |
| 124 | 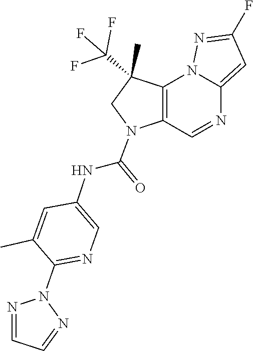 [SMILES: Cc1cc(NC(=O)N2C[C@](C)(C(F)(F)F)c3c2cnc2cc(F)nn32)cnc1-n1nccn1] |
| 125 | 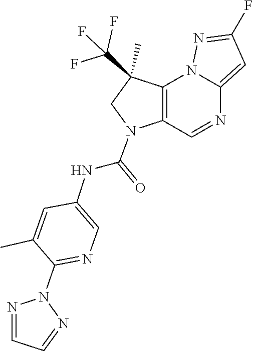 [SMILES: Cc1cc(NC(=O)N2C[C@@](C)(C(F)(F)F)c3c2cnc2cc(F)nn32)cnc1-n1nccn1] |
| 126 | 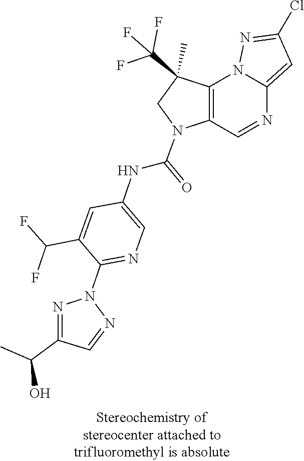 |
| 127 | 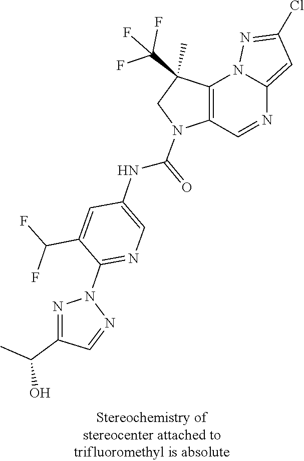 |
| 128 | 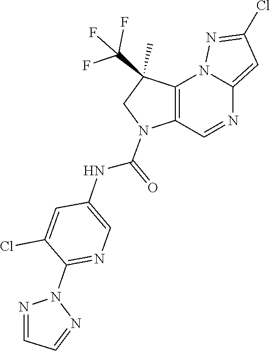 |
| 129 | 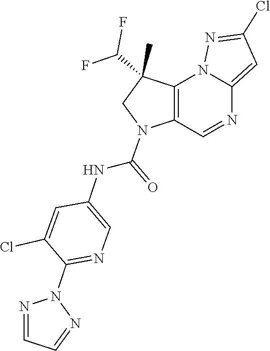 [SMILES: C[C@]1(C(F)F)CN(C(=O)Nc2cnc(-n3nccn3)c(Cl)c2)c2cnc3cc(Cl)nn3c21] |
| 130 | 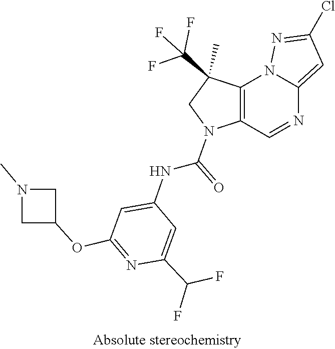 [SMILES: CN1CC(Oc2cc(NC(=O)N3C[C@@](C)(C(F)(F)F)c4c3cnc3cc(Cl)nn43)cc(C(F)F)n2)C1] |
| 131 | 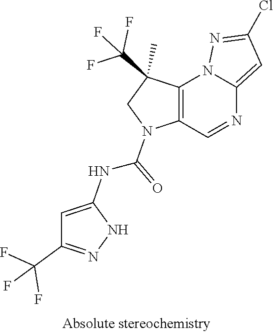 [SMILES: C[C@@]1(C(F)(F)F)CN(C(=O)Nc2cc(C(F)(F)F)n[nH]2)c2cnc3cc(Cl)nn3c21] |
| 132 | 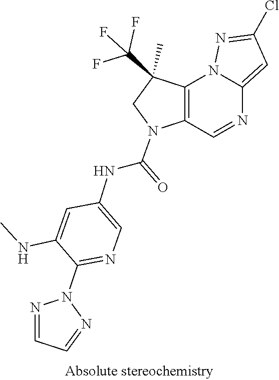 [SMILES: CNc1cc(NC(=O)N2C[C@@](C)(C(F)(F)F)c3c2cnc2cc(Cl)nn32)cnc1-n1nccn1] |
| 133 | 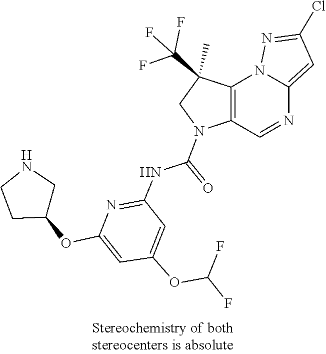 [SMILES: C[C@@]1(C(F)(F)F)CN(C(=O)Nc2cc(OC(F)F)cc(O[C@H]3CCNC3)n2)c2cnc3cc(Cl)nn3c21] |
| 134 | 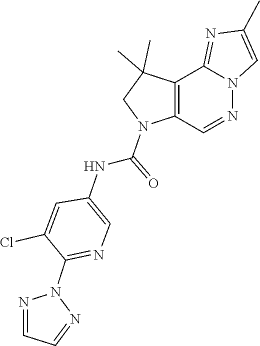 |
| 135 | 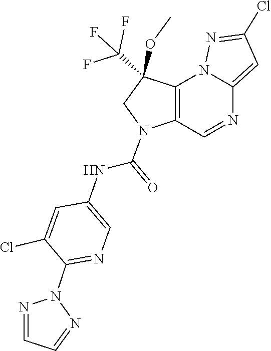 [SMILES: CO[C@]1(C(F)(F)F)CN(C(=O)Nc2cnc(-n3nccn3)c(Cl)c2)c2cnc3cc(Cl)nn3c21] |
| 136 | 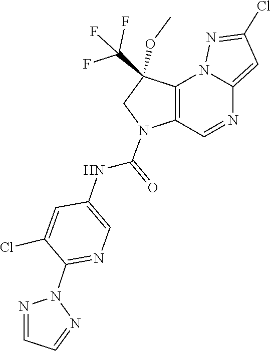 [SMILES: CO[C@@]1(C(F)(F)F)CN(C(=O)Nc2cnc(-n3nccn3)c(Cl)c2)c2cnc3cc(Cl)nn3c21] |
| 137 | 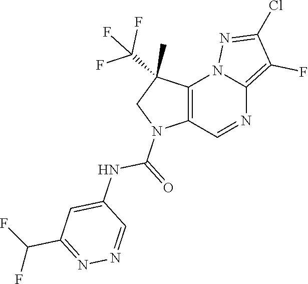 [SMILES: C[C@]1(C(F)(F)F)CN(C(=O)Nc2cnnc(C(F)F)c2)c2cnc3c(F)c(Cl)nn3c21] |
| 138 | 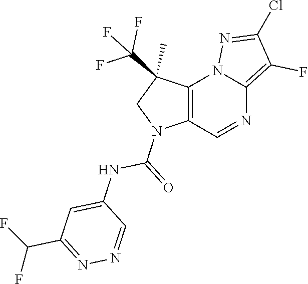 [SMILES: C[C@@]1(C(F)(F)F)CN(C(=O)Nc2cnnc(C(F)F)c2)c2cnc3c(F)c(Cl)nn3c21] |
| 139 | 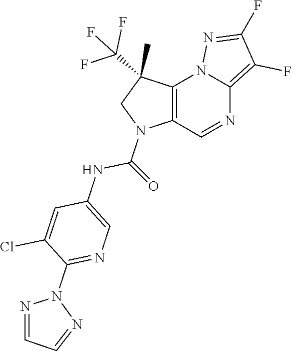 |
| 140 | 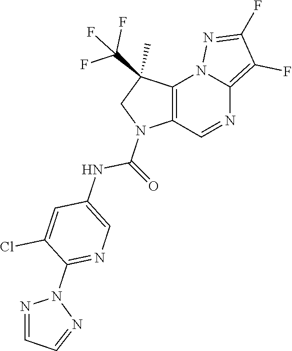 |
| 141 | 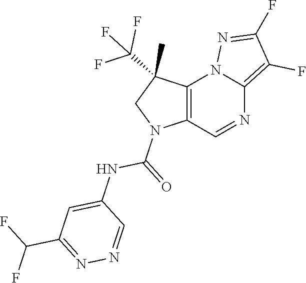 [SMILES: C[C@]1(C(F)(F)F)CN(C(=O)Nc2cnnc(C(F)F)c2)c2cnc3c(F)c(F)nn3c21] |
| 142 | 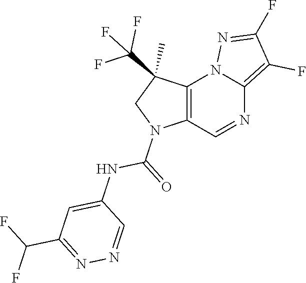 [SMILES: C[C@@]1(C(F)(F)F)CN(C(=O)Nc2cnnc(C(F)F)c2)c2cnc3c(F)c(F)nn3c21] |
| 143 | 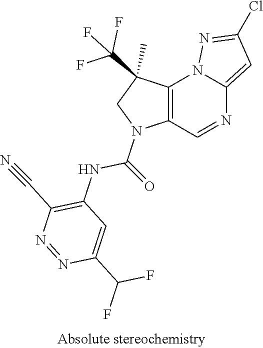 [SMILES: C[C@@]1(C(F)(F)F)CN(C(=O)Nc2cc(C(F)F)nnc2C#N)c2cnc3cc(Cl)nn3c21] |
| 144 | 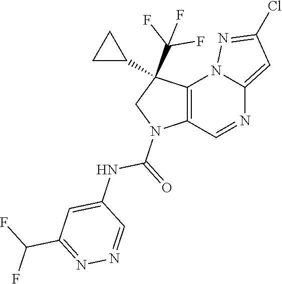 [SMILES: O=C(Nc1cnnc(C(F)F)c1)N1C[C@@](C2CC2)(C(F)(F)F)c2c1cnc1cc(Cl)nn21] |
| 145 | 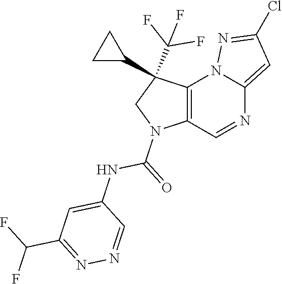 [SMILES: O=C(Nc1cnnc(C(F)F)c1)N1C[C@](C2CC2)(C(F)(F)F)c2c1cnc1cc(Cl)nn21] |
| 146 | 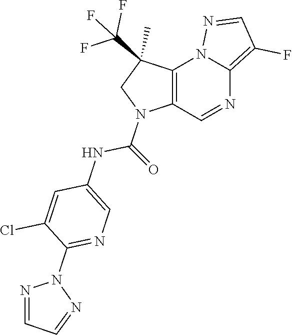 |
| 147 | 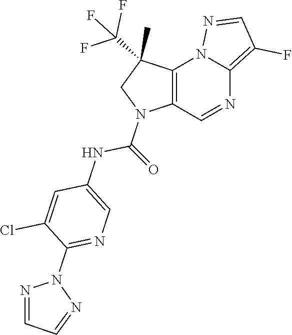 |
| 148 | 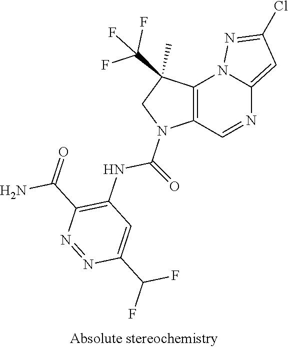 [SMILES: C[C@@]1(C(F)(F)F)CN(C(=O)Nc2cc(C(F)F)nnc2C(N)=O)c2cnc3cc(Cl)nn3c21] |
| 149 | 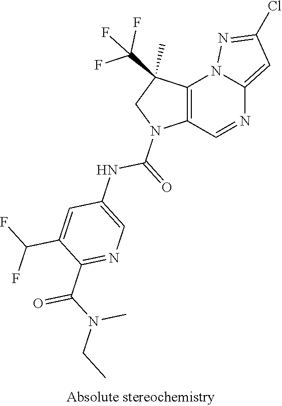 [SMILES: CCN(C)C(=O)c1ncc(NC(=O)N2C[C@@](C)(C(F)(F)F)c3c2cnc2cc(Cl)nn32)cc1C(F)F] |
| 150 | 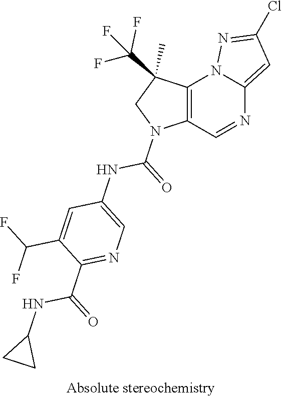 [SMILES: C[C@@]1(C(F)(F)F)CN(C(=O)Nc2cnc(C(=O)NC3CC3)c(C(F)F)c2)c2cnc3cc(Cl)nn3c21] |
| 151 | 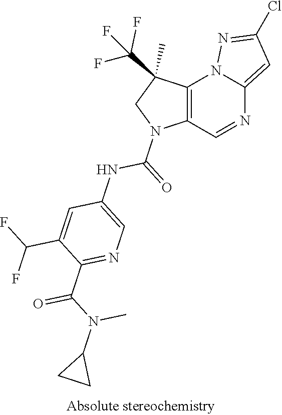 [SMILES: CN(C(=O)c1ncc(NC(=O)N2C[C@@](C)(C(F)(F)F)c3c2cnc2cc(Cl)nn32)cc1C(F)F)C1CC1] |
| 152 | 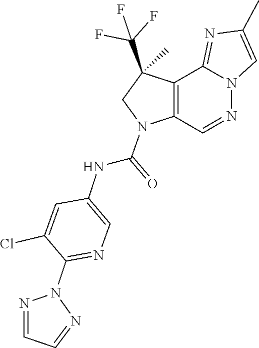 |
| 153 | 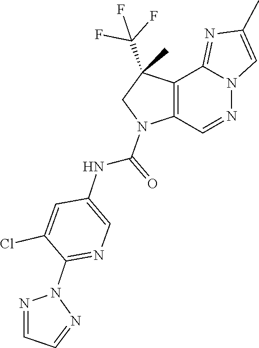 |
| 154 | 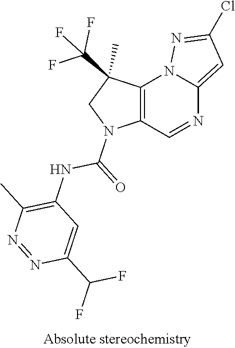 [SMILES: Cc1nnc(C(F)F)cc1NC(=O)N1C[C@@](C)(C(F)(F)F)c2c1cnc1cc(Cl)nn21] |
| 155 | 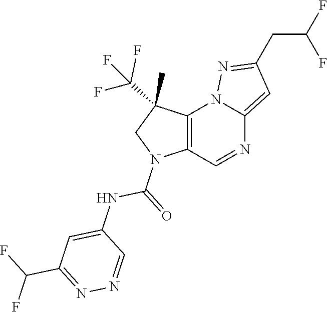 [SMILES: C[C@]1(C(F)(F)F)CN(C(=O)Nc2cnnc(C(F)F)c2)c2cnc3cc(CC(F)F)nn3c21] |
| 156 | 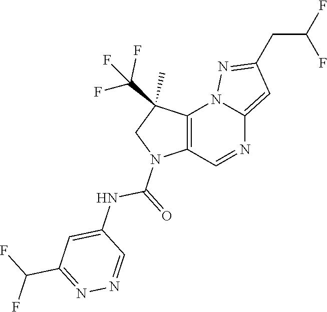 [SMILES: C[C@@]1(C(F)(F)F)CN(C(=O)Nc2cnnc(C(F)F)c2)c2cnc3cc(CC(F)F)nn3c21] |
| 157 | 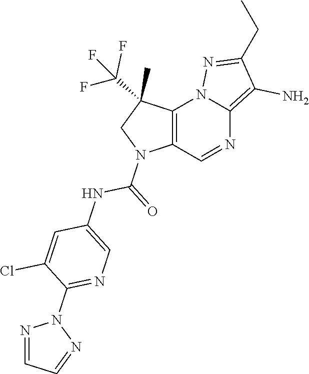 |
| 158 | 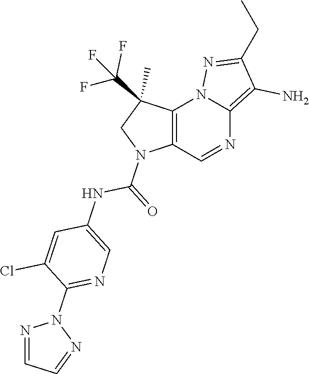 |
| 159 | 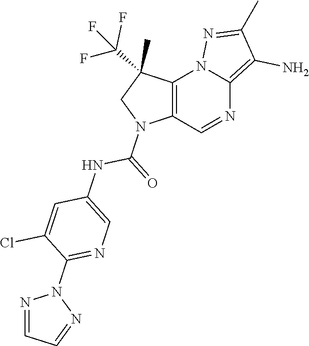 |
| 160 | 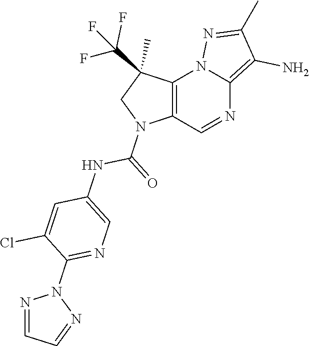 |
| 161 | 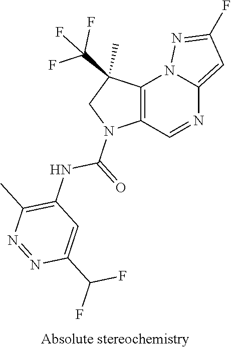 [SMILES: Cc1nnc(C(F)F)cc1NC(=O)N1C[C@@](C)(C(F)(F)F)c2c1cnc1cc(F)nn21] |
| 162 | 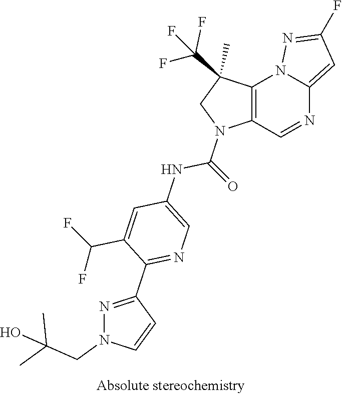 [SMILES: CC(C)(O)Cn1ccc(-c2ncc(NC(=O)N3C[C@@](C)(C(F)(F)F)c4c3cnc3cc(F)nn43)cc2C(F)F)n1] |
| 163 | 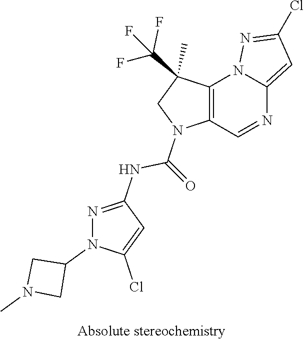 [SMILES: CN1CC(n2nc(NC(=O)N3C[C@@](C)(C(F)(F)F)c4c3cnc3cc(Cl)nn43)cc2Cl)C1] |
| 164 | 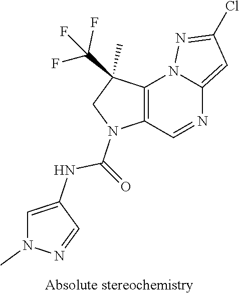 [SMILES: Cn1cc(NC(=O)N2C[C@@](C)(C(F)(F)F)c3c2cnc2cc(Cl)nn32)cn1] |
| 165 | 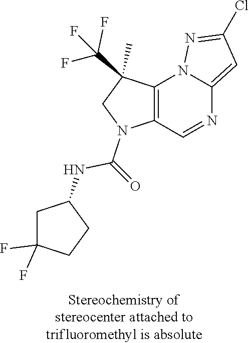 [SMILES: C[C@@]1(C(F)(F)F)CN(C(=O)N[C@@H]2CCC(F)(F)C2)c2cnc3cc(Cl)nn3c21] |
| 166 | 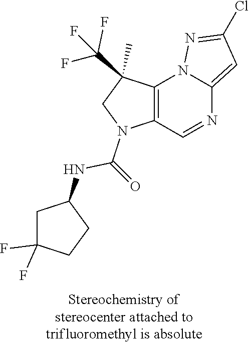 [SMILES: C[C@@]1(C(F)(F)F)CN(C(=O)N[C@H]2CCC(F)(F)C2)c2cnc3cc(Cl)nn3c21] |
| 167 | 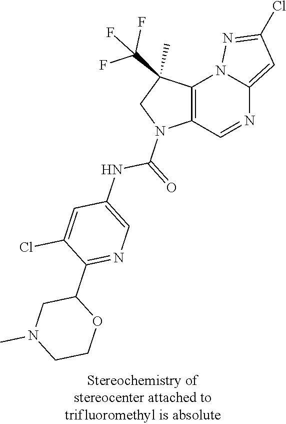 [SMILES: CN1CCOC(c2ncc(NC(=O)N3C[C@@](C)(C(F)(F)F)c4c3cnc3cc(Cl)nn43)cc2Cl)C1] |
| 168 | 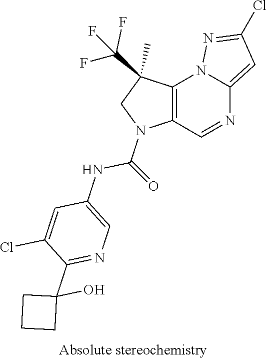 [SMILES: C[C@@]1(C(F)(F)F)CN(C(=O)Nc2cnc(C3(O)CCC3)c(Cl)c2)c2cnc3cc(Cl)nn3c21] |
| 169 | 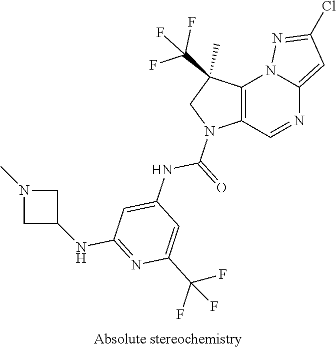 [SMILES: CN1CC(Nc2cc(NC(=O)N3C[C@@](C)(C(F)(F)F)c4c3cnc3cc(Cl)nn43)cc(C(F)(F)F)n2)C1] |
| 170 | 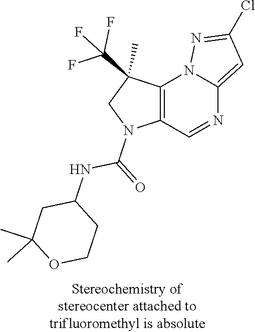 [SMILES: CC1(C)CC(NC(=O)N2C[C@@](C)(C(F)(F)F)c3c2cnc2cc(Cl)nn32)CCO1] |
| 171 | 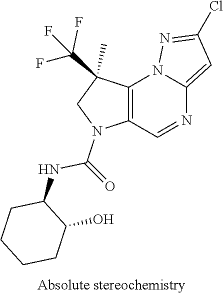 [SMILES: C[C@@]1(C(F)(F)F)CN(C(=O)N[C@@H]2CCCC[C@H]2O)c2cnc3cc(Cl)nn3c21] |
| 172 | 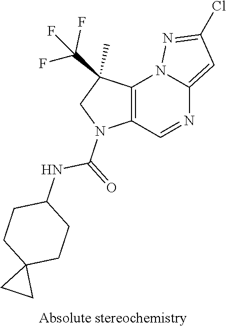 [SMILES: C[C@@]1(C(F)(F)F)CN(C(=O)NC2CCC3(CC2)CC3)c2cnc3cc(Cl)nn3c21] |
| 173 | 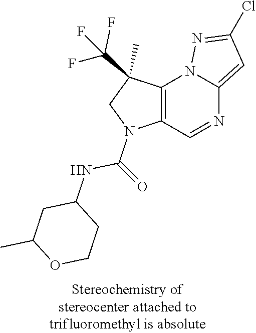 [SMILES: CC1CC(NC(=O)N2C[C@@](C)(C(F)(F)F)c3c2cnc2cc(Cl)nn32)CCO1] |
| 174 | 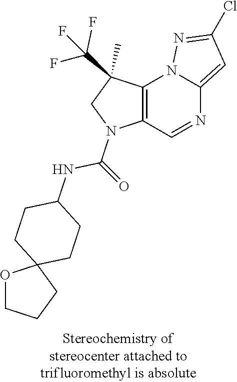 [SMILES: C[C@@]1(C(F)(F)F)CN(C(=O)NC2CCC3(CCCO3)CC2)c2cnc3cc(Cl)nn3c21] |
| 175 | 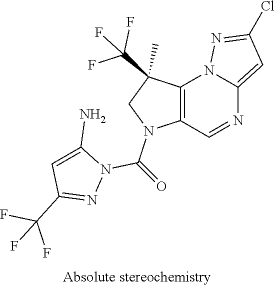 [SMILES: C[C@@]1(C(F)(F)F)CN(C(=O)n2nc(C(F)(F)F)cc2N)c2cnc3cc(Cl)nn3c21] |
| 176 | 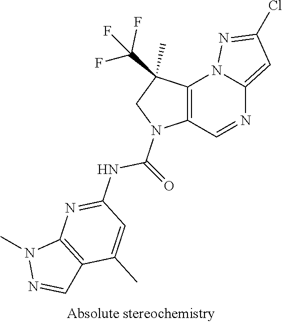 [SMILES: Cc1cc(NC(=O)N2C[C@@](C)(C(F)(F)F)c3c2cnc2cc(Cl)nn32)nc2c1cnn2C] |
| 177 | 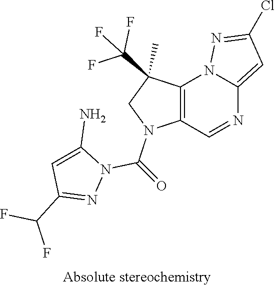 [SMILES: C[C@@]1(C(F)(F)F)CN(C(=O)n2nc(C(F)F)cc2N)c2cnc3cc(Cl)nn3c21] |
| 178 | 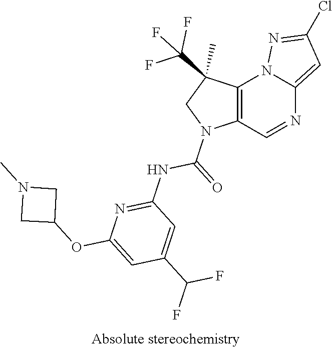 [SMILES: CN1CC(Oc2cc(C(F)F)cc(NC(=O)N3C[C@@](C)(C(F)(F)F)c4c3cnc3cc(Cl)nn43)n2)C1] |
| 179 | 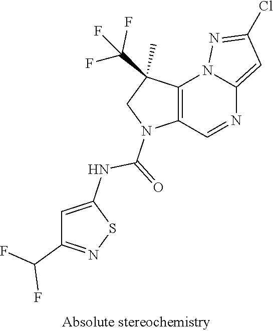 [SMILES: C[C@@]1(C(F)(F)F)CN(C(=O)Nc2cc(C(F)F)ns2)c2cnc3cc(Cl)nn3c21] |
| 180 | 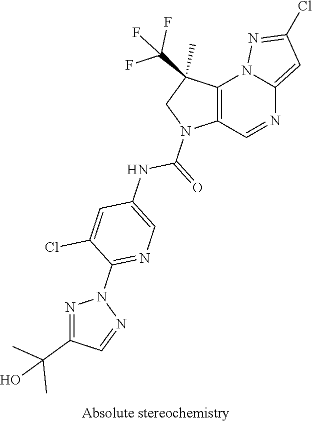 [SMILES: CC(C)(O)c1cnn(-c2ncc(NC(=O)N3C[C@@](C)(C(F)(F)F)c4c3cnc3cc(Cl)nn43)cc2Cl)n1] |
| 181 | 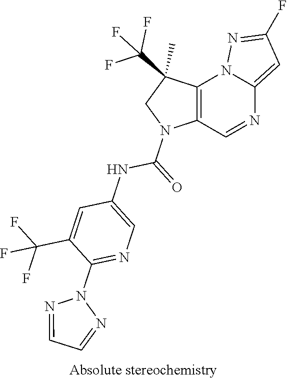 |
| 182 | 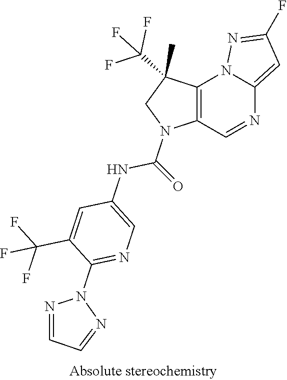 |
| 183 | 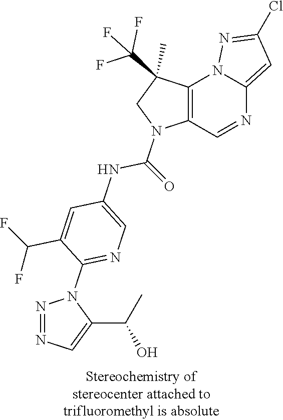 [SMILES: C[C@H](O)c1cnnn1-c1ncc(NC(=O)N2C[C@@](C)(C(F)(F)F)c3c2cnc2cc(Cl)nn32)cc1C(F)F] |
| 184 | 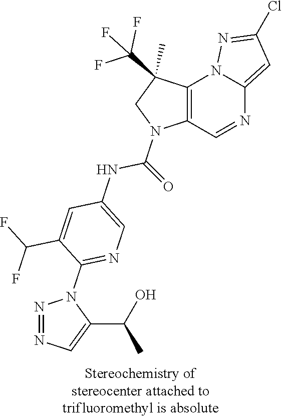 [SMILES: C[C@H](O)c1cnnn1-c1ncc(NC(=O)N2C[C@@](C)(C(F)(F)F)c3c2cnc2cc(Cl)nn32)cc1C(F)F] |
| 185 | 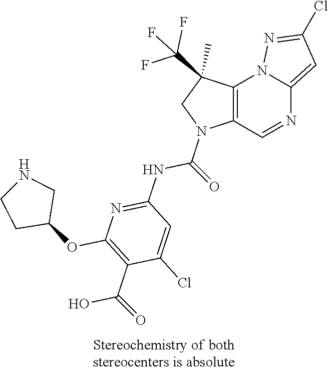 [SMILES: C[C@@]1(C(F)(F)F)CN(C(=O)Nc2cc(Cl)c(C(=O)O)c(O[C@H]3CCNC3)n2)c2cnc3cc(Cl)nn3c21] |
| 186 | 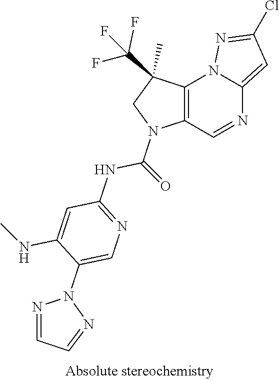 |
| 187 | 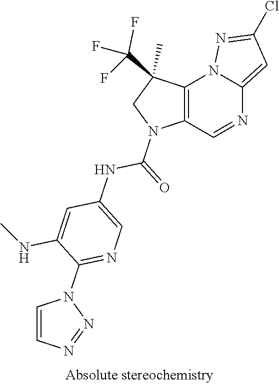 [SMILES: CNc1cc(NC(=O)N2C[C@@](C)(C(F)(F)F)c3c2cnc2cc(Cl)nn32)cnc1-n1ccnn1] |
| 188 | 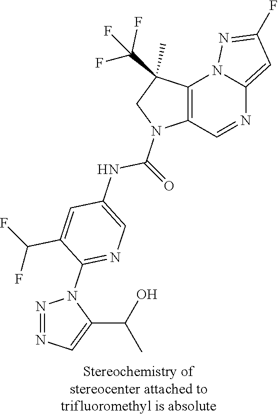 [SMILES: CC(O)c1cnnn1-c1ncc(NC(=O)N2C[C@@](C)(C(F)(F)F)c3c2cnc2cc(F)nn32)cc1C(F)F] |
| 189 | 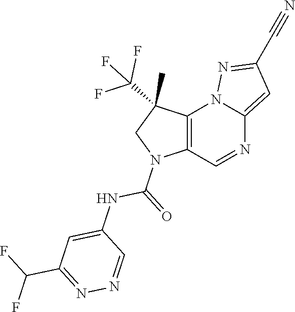 [SMILES: C[C@]1(C(F)(F)F)CN(C(=O)Nc2cnnc(C(F)F)c2)c2cnc3cc(C#N)nn3c21] |
| 190 | 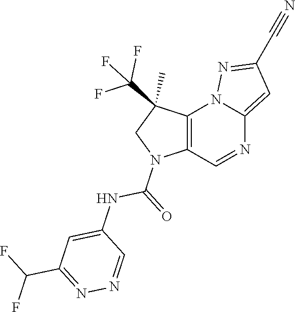 [SMILES: C[C@@]1(C(F)(F)F)CN(C(=O)Nc2cnnc(C(F)F)c2)c2cnc3cc(C#N)nn3c21] |
| 191 | 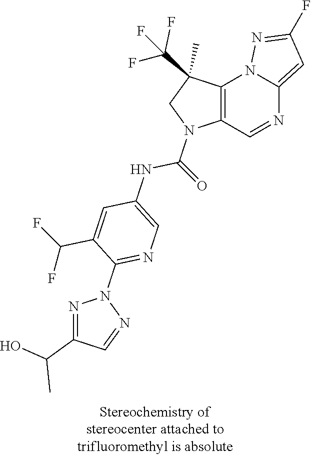 [SMILES: CC(O)c1cnn(-c2ncc(NC(=O)N3C[C@@](C)(C(F)(F)F)c4c3cnc3cc(F)nn43)cc2C(F)F)n1] |
| 192 | 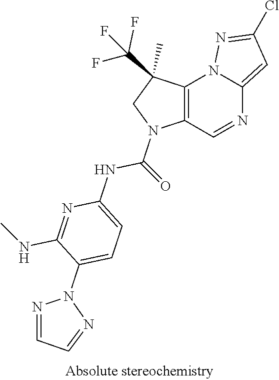 |
| 193 | 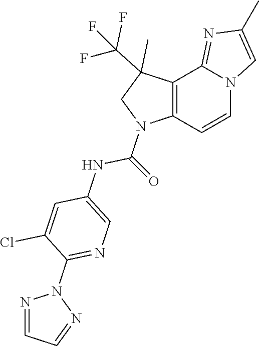 [SMILES: Cc1cn2ccc3c(c2n1)C(C)(C(F)(F)F)CN3C(=O)Nc1cnc(-n2nccn2)c(Cl)c1] |
| 194 | 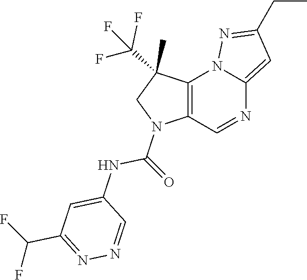 [SMILES: CCc1cc2ncc3c(n2n1)[C@@](C)(C(F)(F)F)CN3C(=O)Nc1cnnc(C(F)F)c1] |
| 195 | 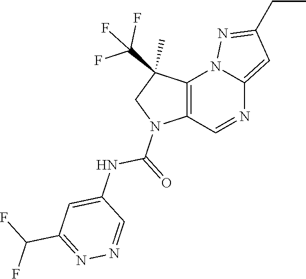 [SMILES: CCc1cc2ncc3c(n2n1)[C@](C)(C(F)(F)F)CN3C(=O)Nc1cnnc(C(F)F)c1] |
| 196 | 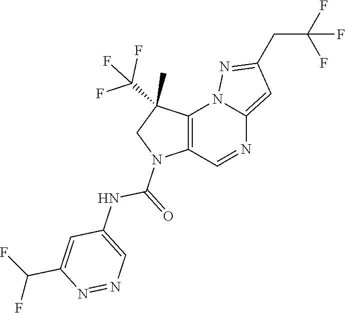 [SMILES: C[C@]1(C(F)(F)F)CN(C(=O)Nc2cnnc(C(F)F)c2)c2cnc3cc(CC(F)(F)F)nn3c21] |
| 197 | 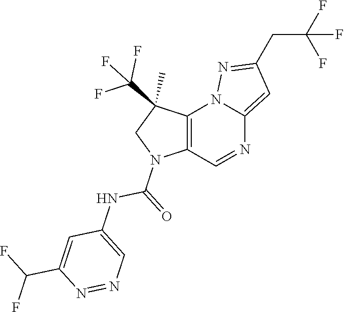 [SMILES: C[C@@]1(C(F)(F)F)CN(C(=O)Nc2cnnc(C(F)F)c2)c2cnc3cc(CC(F)(F)F)nn3c21] |
| 198 | 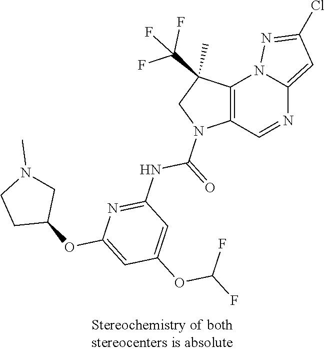 [SMILES: CN1CC[C@H](Oc2cc(OC(F)F)cc(NC(=O)N3C[C@@](C)(C(F)(F)F)c4c3cnc3cc(Cl)nn43)n2)C1] |
| 199 | 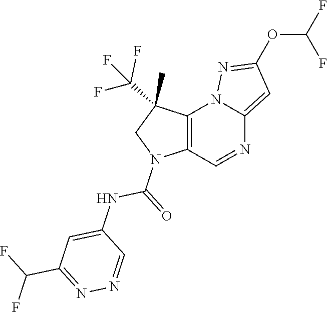 [SMILES: C[C@]1(C(F)(F)F)CN(C(=O)Nc2cnnc(C(F)F)c2)c2cnc3cc(OC(F)F)nn3c21] |
| 200 | 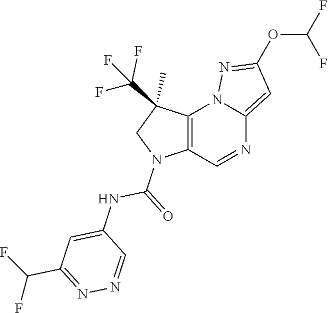 [SMILES: C[C@@]1(C(F)(F)F)CN(C(=O)Nc2cnnc(C(F)F)c2)c2cnc3cc(OC(F)F)nn3c21] |
| 201 | 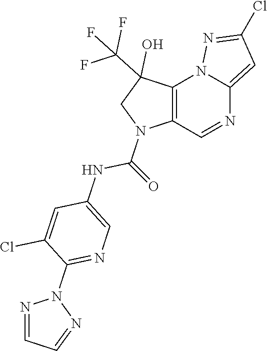 [SMILES: O=C(Nc1cnc(-n2nccn2)c(Cl)c1)N1CC(O)(C(F)(F)F)c2c1cnc1cc(Cl)nn21] |
| 202 | 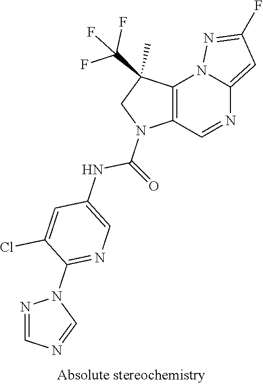 [SMILES: C[C@@]1(C(F)(F)F)CN(C(=O)Nc2cnc(-n3cncn3)c(Cl)c2)c2cnc3cc(F)nn3c21] |
| 203 | 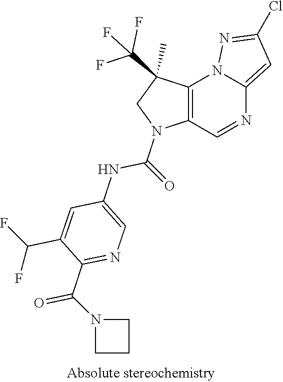 [SMILES: C[C@@]1(C(F)(F)F)CN(C(=O)Nc2cnc(C(=O)N3CCC3)c(C(F)F)c2)c2cnc3cc(Cl)nn3c21] |
| 204 | 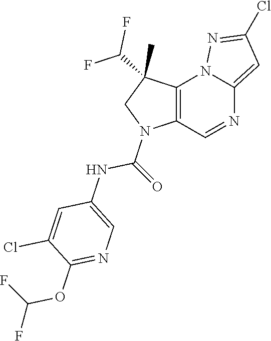 [SMILES: C[C@]1(C(F)F)CN(C(=O)Nc2cnc(OC(F)F)c(Cl)c2)c2cnc3cc(Cl)nn3c21] |
| 205 | 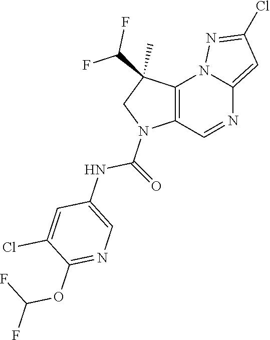 [SMILES: C[C@@]1(C(F)F)CN(C(=O)Nc2cnc(OC(F)F)c(Cl)c2)c2cnc3cc(Cl)nn3c21] |
| 206 | 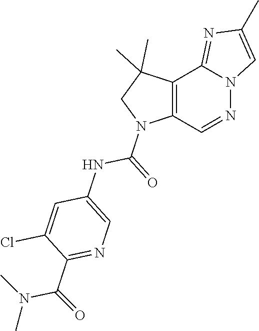 |
| 207 | 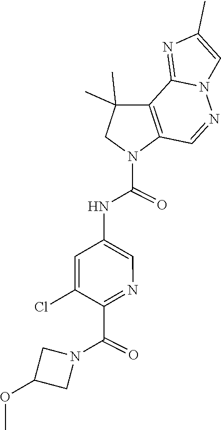 [SMILES: COC1CN(C(=O)c2ncc(NC(=O)N3CC(C)(C)c4c3cnn3cc(C)nc43)cc2Cl)C1] |
| 208 | 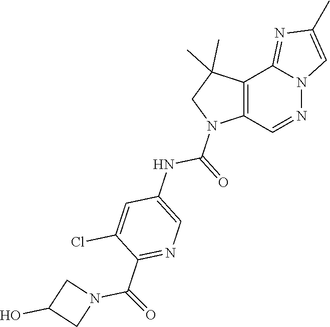 [SMILES: Cc1cn2ncc3c(c2n1)C(C)(C)CN3C(=O)Nc1cnc(C(=O)N2CC(O)C2)c(Cl)c1] |
| 209 | 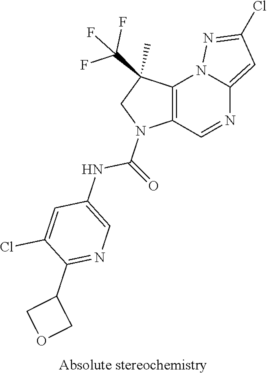 [SMILES: C[C@@]1(C(F)(F)F)CN(C(=O)Nc2cnc(C3COC3)c(Cl)c2)c2cnc3cc(Cl)nn3c21] |
| 210 | 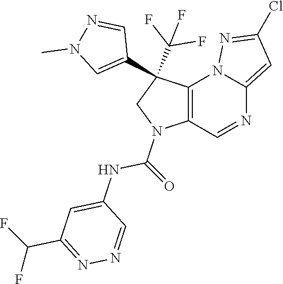 [SMILES: Cn1cc([C@]2(C(F)(F)F)CN(C(=O)Nc3cnnc(C(F)F)c3)c3cnc4cc(Cl)nn4c32)cn1] |
| 211 | 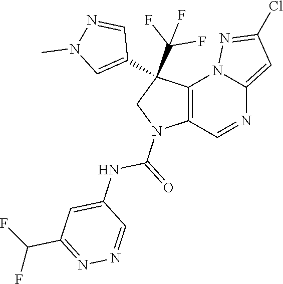 [SMILES: Cn1cc([C@@]2(C(F)(F)F)CN(C(=O)Nc3cnnc(C(F)F)c3)c3cnc4cc(Cl)nn4c32)cn1] |

### Processes of Preparation

Provided herein is a process of preparing a compound of Formula (I) (e.g., any compound described herein), comprising:
reacting a compound of Formula (I-A)

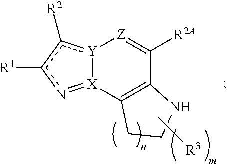

with R⁴—NH₂;
to form a compound of Formula (I).

In some embodiments, reacting the compound of Formula (I-A) with R⁴—NH₂ comprises reacting one of the compounds of Formula (I-A) and R⁴—NH₂ with a carbonyl equivalent to form an intermediate, then reacting the other of the compounds of Formula (I-A) and R⁴—NH₂ with the intermediate. In some of these embodiments, reacting the compound of Formula (I-A) with R⁴—NH₂ comprises reacting R⁴—NH₂ with a carbonyl equivalent to form the intermediate, then reacting the compound of Formula (I-A) with the intermediate. In any of the foregoing embodiments, “carbonyl equivalent” refers to a reagent that replaces an N—H group in the compound of Formula (I-A) and/or R⁴—NH₂ with a carbonyl moiety. Non-limiting examples of carbonyl equivalents include triphosgene and bis(trichloromethyl)carbonate.

In some embodiments, reacting the compound of Formula (I-A) with R⁴—NH₂ comprises reacting one of the compounds of Formula (I-A) and R⁴—NH₂ with a carbonyl equivalent selected from triphosgene and bis(trichloromethyl)carbonate to form an intermediate, then reacting the other of the compounds of Formula (I-A) and R⁴—NH₂ with the intermediate. In some of these embodiments, reacting the compound of Formula (I-A) with R⁴—NH₂ comprises reacting R⁴—NH₂ with a carbonyl equivalent selected from triphosgene and bis(trichloromethyl)carbonate to form the intermediate, then reacting the compound of Formula (I-A) with the intermediate. In some embodiments, the carbonyl equivalent is triphosgene. In some embodiments, the carbonyl equivalent is bis(trichloromethyl)carbonate.

Provided herein is a process of preparing a compound of Formula (I) (e.g., any compound described herein), comprising:
reacting a compound of Formula (I-A)

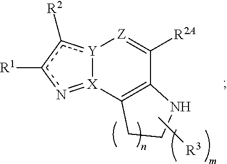

with R⁴—C(O)OH;
to form a compound of Formula (I).

In some embodiments, reacting the compound of Formula (I-A) with R⁴—C(O)OH comprises reacting R⁴—C(O)OH with diphenylphoshoryl azide (e.g., to form an intermediate (e.g., R⁴—C(O)N₃)) then heating (to, e.g., form a second intermediate (e.g., R⁴—N═C═O)) in the presence of the compound of Formula (I-A) to form the compound of Formula (I)

In some embodiments, the compound of Formula (I-A) is a compound of Formula (I-A-N):

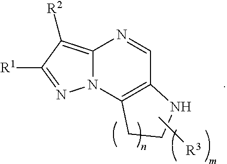

In some embodiments, when the compound of Formula (I-A) is a compound of Formula (I-A-N), the process further comprises reacting a compound of Formula (I-A-N-i)

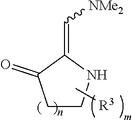

with a compound of Formula (I-A-N-ii)

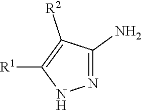

to form the compound of Formula (I-A-N).

In certain embodiments, reacting the compound of Formula (I-A-N-i) with the compound of Formula (I-A-N-ii) is performed in the presence of acid, such as an organic or inorganic acid. In some embodiments, the acid is hydrochloric acid or acetic acid.

In some embodiments, the compound of Formula (I-A) is a compound of Formula (I-A-M):

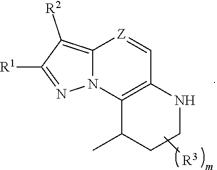

In some embodiments, when the compound of Formula (I-A) is a compound of Formula (I-A-M), the process further comprises reacting a compound of Formula (I-A-M-i)

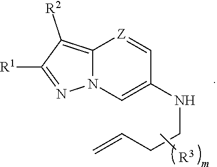

to form the compound of Formula (I-A-M).

In some of these embodiments, the compound of Formula (I-A-M-i) is reacted with an iron salt, a silane, a peroxide, and an acid to form the compound of Formula (I-A-M). In some embodiments, the iron salt is ferric (Z)-4-oxopent-2-en-2-olate. In some embodiments, the silane is phenylsilane. In some embodiments, the peroxide is 2-tert-butylperoxy-2-methyl-propane. In some embodiments, the acid is 2,2,2-trifluoroacetic acid.

Methods of Treatment

Some embodiments provide a method of treating an autoimmune disorder (e.g., a MALT1-associated autoimmune disorder) in a subject in need of such treatment, the method comprising administering to the subject an effective amount of a compound of Formula (I), or a pharmaceutically acceptable salt thereof or a pharmaceutical composition thereof. In some embodiments, the autoimmune disorder is rheumatoid arthritis, multiple sclerosis, or systemic lupus erythematosus (SLE).

Some embodiments provide a method of treating an inflammatory disorder (e.g., a MALT1-associated inflammatory disorder) in a subject in need of such treatment, the method comprising administering to the subject an effective amount of a compound of Formula (I), or a pharmaceutically acceptable salt thereof or a pharmaceutical composition thereof. In some embodiments, the inflammatory disorder is chronic graft versus host disease (cGVHD).

Some embodiments provide a method of treating cancer (e.g., a MALT1-associated cancer) in a subject in need of such treatment, the method comprising administering to the subject an effective amount of a compound of Formula (I), or a pharmaceutically acceptable salt thereof or a pharmaceutical composition thereof. For example, provided herein are methods for treating a MALT1-associated cancer in a subject in need of such treatment, the method comprising a) detecting a dysregulation of a MALT1 gene, a MALT1 protease, or the expression or activity or level of any of the same in a sample from the subject; and b) administering an effective amount of a compound of Formula (I), or a pharmaceutically acceptable salt thereof. In some embodiments, the dysregulation of a MALT1 gene, a MALT1 protease, or the expression or activity or level of any of the same includes one or more fusion proteins.

In some embodiments of any of the methods or uses described herein, the cancer (e.g., MALT1-associated cancer) is a hematological cancer. In some embodiments of any of the methods or uses described herein, the cancer (e.g., MALT1-associated cancer) is a solid tumor. In some embodiments of any of the methods or uses described herein, the cancer (e.g., MALT1-associated cancer) is a lung cancer (e.g., small cell lung carcinoma or non-small cell lung carcinoma), thyroid cancer (e.g., papillary thyroid cancer, medullary thyroid cancer (e.g., sporadic medullary thyroid cancer or hereditary medullary thyroid cancer), differentiated thyroid cancer, recurrent thyroid cancer, or refractory differentiated thyroid cancer), thyroid adenoma, endocrine gland neoplasms, lung adenocarcinoma, bronchioles lung cell carcinoma, multiple endocrine neoplasia type 2A or 2B (MEN2A or MEN2B, respectively), pheochromocytoma, parathyroid hyperplasia, breast cancer, mammary cancer, mammary carcinoma, mammary neoplasm, colorectal cancer (e.g., metastatic colorectal cancer), papillary renal cell carcinoma, ganglioneuromatosis of the gastroenteric mucosa, inflammatory myofibroblastic tumor, or cervical cancer. In some embodiments of any of the methods or uses described herein, the cancer (e.g., MALT1-associated cancer) is selected from the group of: acute lymphoblastic leukemia (ALL), acute myeloid leukemia (AML), cancer in adolescents, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, unknown primary carcinoma, cardiac tumors, cervical cancer, childhood cancers, chordoma, chronic lymphocytic leukemia (CLL), chronic myelogenous leukemia (CML), chronic myeloproliferative neoplasms, neoplasms by site, neoplasms, colon cancer, colorectal cancer, craniopharyngioma, cutaneous T-cell lymphoma, cutaneous angiosarcoma, bile duct cancer, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, extrahepatic bile duct cancer, eye cancer, fallopian tube cancer, fibrous histiocytoma of bone, gallbladder cancer, gastric cancer, gastrointestinal carcinoid tumor, gastrointestinal stromal tumors (GIST), germ cell tumor, gestational trophoblastic disease, glioma, hairy cell tumor, hairy cell leukemia, head and neck cancer, thoracic neoplasms, head and neck neoplasms, CNS tumor, primary CNS tumor, heart cancer, hepatocellular cancer, histiocytosis, Hodgkin's lymphoma, hypopharyngeal cancer, intraocular melanoma, islet cell tumors, pancreatic neuroendocrine tumors, Kaposi sarcoma, kidney cancer, Langerhans cell histiocytosis, laryngeal cancer, leukemia, lip and oral cavity cancer, liver cancer, lung cancer, lymphoma, macroglobulinemia, malignant fibrous histiocytoma of bone, osteocarcinoma, melanoma, Merkel cell carcinoma, mesothelioma, metastatic squamous neck cancer, midline tract carcinoma, mouth cancer, multiple endocrine neoplasia syndromes, multiple myeloma, mycosis fungoides, myelodysplastic syndromes, myelodysplastic/myeloproliferative neoplasms, neoplasms by site, neoplasms, myelogenous leukemia, myeloid leukemia, multiple myeloma, myeloproliferative neoplasms, nasal cavity and paranasal sinus cancer, nasopharyngeal cancer, neuroblastoma, non-Hodgkin's lymphoma, non-small cell lung cancer, lung neoplasm, pulmonary cancer, pulmonary neoplasms, respiratory tract neoplasms, bronchogenic carcinoma, bronchial neoplasms, oral cancer, oral cavity cancer, lip cancer, oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromosytoma, pituitary cancer, plasma cell neoplasm, pleuropulmonary blastoma, pregnancy-associated breast cancer, primary central nervous system lymphoma, primary peritoneal cancer, prostate cancer, rectal cancer, colon cancer, colonic neoplasms, renal cell cancer, rhabdomyosarcoma, salivary gland cancer, sarcoma, Sezary syndrome, skin cancer, Spitz tumors, small cell lung cancer, small intestine cancer, soft tissue sarcoma, squamous cell carcinoma, squamous neck cancer, stomach cancer, T-cell lymphoma, testicular cancer, throat cancer, thymoma and thymic carcinoma, thyroid cancer, transitional cell cancer of the renal pelvis, unknown primary carcinoma, urethral cancer, uterine cancer, uterine sarcoma, vaginal cancer, vulvar cancer, and Wilms' tumor.

In some embodiments, the cancer is a hematological cancer, such as a leukemia or a lymphoma. In some embodiments, a hematological cancer (e.g., hematological cancers that are MALT1-associated cancers) is selected from the group consisting of leukemias, lymphomas (non-Hodgkin's lymphoma), Hodgkin's disease (also called Hodgkin's lymphoma), and myeloma, for instance, acute lymphocytic leukemia (ALL), acute myeloid leukemia (AML), acute promyelocytic leukemia (APL), chronic lymphocytic leukemia (CLL), chronic myeloid leukemia (CML), chronic myelomonocytic leukemia (CMML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AMLiTMDS), mixed lineage leukemia (MLL), myelodysplastic syndromes (MDSs), myeloproliferative disorders (MPD), and multiple myeloma (MM). Additional examples of hematological cancers include myeloproliferative disorders (MPD) such as polycythemia vera (PV), essential thrombocytopenia (ET) and idiopathic primary myelofibrosis (IMF/IPF/PMF). In some embodiments, the hematological cancer (e.g., the hematological cancer that is a MALT1-associated cancer) is AML or CMML.

In some embodiments, the cancer is glioblastoma, chronic myelogenous leukemia, myeloid leukemia, or non-Hodgkin's lymphoma.

In some embodiments, the cancer (e.g., the MALT1-associated cancer) is a solid tumor. Examples of solid tumors (e.g., solid tumors that are MALT1-associated cancers) include, for example, lung cancer (e.g., lung adenocarcinoma, small-cell lung carcinoma), pancreatic cancer, pancreatic ductal carcinoma, breast cancer, colon cancer, colorectal cancer, prostate cancer, renal cell carcinoma, neuroblastoma, and melanoma. See. e.g., Jiang et al., Cancer Research 2011, 71, 2183-2192; see also. Pan et al., Mol Cancer Res 2016, 14, 93-102 and Penas et al., Blood 2010, 115, 2214-2219.

In some embodiments, the subject is a human.

Compounds of Formula (I) and pharmaceutically acceptable salts thereof are also useful for treating a MALT1-associated cancer. Compounds of Formula (I) and pharmaceutically acceptable salts thereof are also useful for treating a MALT1-associated autoimmune disorder. Compounds of Formula (I) and pharmaceutically acceptable salts thereof are also useful for treating a MALT1-associated inflammatory disease.

Accordingly, also provided herein is a method for treating a subject diagnosed with or identified as having a MALT1-associated cancer, e.g., any of the exemplary MALT1-associated cancers disclosed herein, comprising administering to the subject an effective amount of a compound of Formula (I), or a pharmaceutically acceptable salt thereof, or a pharmaceutical composition thereof as defined herein.

In some embodiments of any of the methods provided herein, a compound of Formula (I) is selected from Examples 1-211.

Dysregulation of a MALT1 protease, a MALT1 gene, or the expression or activity or level of any (e.g., one or more) of the same can contribute to tumorigenesis. For example, a fusion protein can have increased protease activity as compared to a wild-type MALT1 protein, increased expression (e.g., increased levels) of a wild-type MALT1 protease in a mammalian cell can occur due to aberrant cell signaling and/or dysregulated autocrine/paracrine signaling (e.g., as compared to a control non-cancerous cell), MALT1 mRNA splice variants may also result in dysregulation of MALT1.

In some aspects, provided herein is a method for treating cancer in a subject in need thereof, including administering to the subject an effective amount of a compound of Formula (I), or a pharmaceutically acceptable salt thereof. Also provided herein is a method for treating a CBM complex pathway-associated cancer (such as any of those disclosed herein) in a subject in need thereof, including administering to the subject an effective amount of a compound of Formula (I), or a pharmaceutically acceptable salt thereof. Also provided is a method for treating a cancer in a subject in need thereof, including (a) identifying the cancer as being a CBM complex pathway-associated cancer; and (b) administering to the subject an effective amount of a compound of Formula (I), or a pharmaceutically acceptable salt thereof.

Identifying the cancer identifying the cancer in the subject as a CBM complex pathway-associated cancer can be performed by any appropriate method. In some embodiments, the step of identifying the cancer in the subject as a CBM complex pathway-associated cancer includes performing an assay to detect dysregulation in a CBM complex pathway-associated gene, a CBM complex pathway-associated protease protein, or expression or activity or level of any of the same in a sample from the subject. In some embodiments, the method further includes obtaining a sample from the subject (e.g., a biopsy sample). An assay can be any appropriate assay. In some embodiments, the assay is selected from the group consisting of sequencing (e.g., pyrosequencing or next generation sequencing), immunohistochemistry, enzyme-linked immunosorbent assay, and fluorescence in situ hybridization (FISH).

Also provided herein is a method for treating a cancer in a subject in need thereof, including administering to the subject an effective amount of a compound of Formula (I), or a pharmaceutically acceptable salt thereof to a subject identified as having a CBM complex pathway-associated cancer.

Also provided herein is a method of treating a MALT1-associated cancer in a subject, including administering to a subject identified or diagnosed as having a MALT1-associated cancer an effective amount of a compound of Formula (I), or a pharmaceutically acceptable salt thereof. Provided herein is also a method for treating cancer in a subject in need thereof, including: (a) determining that the cancer is associated with a dysregulation of a MALT1 gene, a MALT1 protease, or expression or activity or level of any of the same; and (b) administering to the subject an effective amount of a compound of Formula (I), or a pharmaceutically acceptable salt thereof.

Determining that the cancer is associated with a dysregulation of a MALT1 gene, a MALT1 protease, or expression or activity or level of any of the same can be performed using any appropriate method. In some embodiments, the step of determining that the cancer in the subject is a MALT1-associated cancer includes performing an assay to detect dysregulation in a MALT1 gene, a MALT1 protease protein, or expression or activity or level of any of the same in a sample from the subject. In some embodiments, the method further includes obtaining a sample from the subject (e.g., a biopsy sample). An assay can be any appropriate assay. In some embodiments, the assay is selected from the group consisting of sequencing (e.g., pyrosequencing or next generation sequencing), immunohistochemistry, enzyme-linked immunosorbent assay, and fluorescence in situ hybridization (FISH).

As described herein, a CBM complex pathway-associated cancer can be any appropriate CBM complex pathway-associated cancer (such as any of those described herein). In some embodiments, a CBM complex pathway-associated cancer is selected from the group consisting of a CBM complex pathway cell surface receptor-associated cancer, a cancer associated with a signal transducer between a cell surface receptor and a CBM complex, a component of a CBM complex-associated cancer, a MALT1 protease substrate-associated cancer, a cancer associated with a component of the NF-κB pathway downstream of a CBM complex, a cancer associated with a component of the JNK pathway downstream of a CBM complex, and a combination thereof. In some embodiments, the CBM complex pathway cell surface receptor-associated cancer is selected from the group consisting of a CD28-associated cancer, a BCR-associated cancer, a HER1-associated cancer, a HER2-associated cancer, and combinations thereof. In some embodiments, the cancer associated with a signal transducer between a cell surface receptor and a CBM complex is a protein kinase C beta (PKCβ)-associated cancer, a protein kinase C theta (PCKθ)-associated cancer, or a combination thereof. In some embodiments, the component of a CBM complex-associated cancer is selected from the group consisting of a MALT1-associated cancer, a CARD11-associated cancer, a CARD14-associated cancer, a CARD10-associated cancer, a CARD9-associated cancer, a BCL10-associated cancer, and combinations thereof. In some embodiments, the component of a CBM complex-associated cancer is selected from the group consisting of a MALT1-associated cancer, a CARD11-associated cancer, a BCL10-associated cancer, and combinations thereof. See, e.g., Tables B1, B2, and B3 for exemplary dysregulations in MALT1, CARD11, and BCL10. In some embodiments, the MALT1 protease substrate-associated cancer is selected from the group consisting of a BCL10-associated cancer, an A20-associated cancer, a CYLD-associated cancer, a RelB-associated cancer, a Regnase 1-associated cancer, a roquin-1-associated cancer, a HOIL1-associated cancer, a NIK associated cancer, a LIMA1α-associated cancer, and a combination thereof. In some embodiments, the MALT1 protease substrate-associated cancer is selected from the group consisting of a BCL10-associated cancer, an A20-associated cancer, a CYLD-associated cancer, and combinations thereof. See, e.g., Tables B3 and B4 for exemplary dysregulations in BCL10 and A20. In some embodiments, the cancer associated with a component of the NF-κB pathway downstream of a CBM complex is selected from the group consisting of a TAK1-associated cancer, a TRAF6-associated cancer, a TAB1-associated cancer, a TAB2-associated cancer, a TAB3-associated cancer, a MKK7-associated cancer, an IKKα-associated cancer, an IKKβ-associated cancer, an IKKγ-associated cancer, an IkBα-associated cancer, a p50-associated cancer, a p65 (RelA)-associated cancer, a c-Rel-associated cancer, and combinations thereof. In some embodiments, the cancer associated with a component of the NF-κB pathway downstream of a CBM complex is an IKKγ-associated cancer. In some embodiments, the cancer associated with a component of the JNK pathway downstream of a CBM complex is selected from the group consisting of a JNK1-associated cancer, a JNK2-associated cancer, a JNK3-associated cancer, a MYD88 transcription factor-associated cancer, an AP-1 transcription factor-associated cancer, and combinations thereof.

In some embodiments, the CBM complex pathway-associated cancer is a MALT1-associated cancer. A MALT1-associated cancer can have any appropriate dysregulation, such as any of those described herein. In some embodiments, the MALT1-associated cancer comprises an IAP2-MALT1 fusion. In some embodiments, the MALT1-associated cancer comprises an IGH-MALT1 fusion.

Also provided herein are methods of treating CBM complex pathway-associated diseases or disorders, autoimmune disorders, and inflammatory disorders. Accordingly, provided herein is a method for treating an autoimmune disorder in a subject in need thereof, including administering to the subject an effective amount of a compound of Formula (I), or a pharmaceutically acceptable salt thereof. Also provided herein is a method of treating a MALT1-associated autoimmune disorder in a subject, including administering to a subject identified or diagnosed as having a MALT1-associated autoimmune disorder an effective amount of a compound of Formula (I), or a pharmaceutically acceptable salt thereof. In some cases, provided herein is a method for treating an autoimmune disorder in a subject in need thereof, including: (a) determining that the autoimmune disorder is associated with a dysregulation of a MALT1 gene, a MALT1 protease, or expression or activity or level of any of the same; and (b) administering to the subject an effective amount of a compound of Formula (I), or a pharmaceutically acceptable salt thereof. Provided also herein is a method of treating a MALT1-associated autoimmune disorder in a subject, including administering an effective amount of a compound of Formula (I), or a pharmaceutically acceptable salt thereof to a subject determined to have a MALT1-associated autoimmune disorder. In addition, provided herein is a method for treating an inflammatory disorder in a subject in need thereof, including administering to the subject an effective amount of a compound of Formula (I), or a pharmaceutically acceptable salt thereof. In some cases, provided herein is a method of treating a MALT1-associated inflammatory disorder in a subject, including administering to a subject identified or diagnosed as having a MALT1-associated inflammatory disorder an effective amount of a compound of Formula (I), or a pharmaceutically acceptable salt thereof. Also provided herein is a method for treating an inflammatory disorder in a subject in need thereof, including: (a) determining that the inflammatory disorder is associated with a dysregulation of a MALT1 gene, a MALT1 protease, or expression or activity or level of any of the same; and (b) administering to the subject an effective amount of a compound of Formula (I), or a pharmaceutically acceptable salt thereof. Provided also herein is a method of treating a MALT1-associated inflammatory disorder in a subject, including administering an effective amount of a compound of Formula (I), or a pharmaceutically acceptable salt thereof to a subject determined to have a MALT1-associated inflammatory disorder Additionally provided herein is a method for treating a CBM complex pathway-associated disease or disorder in a subject in need thereof, including administering to the subject an effective amount of a compound of Formula (I), or a pharmaceutically acceptable salt thereof. Also provided is a method for treating a disease or disorder in a subject in need thereof, including: (a) identifying the cancer as being a CBM complex pathway-associated disease or disorder; and (b) administering to the subject an effective amount of a compound of Formula (I), or a pharmaceutically acceptable salt thereof. In addition, provided herein is a method for treating a disease or disorder in a subject in need thereof, including: administering to the subject an effective amount of a compound of Formula (I), or a pharmaceutically acceptable salt thereof to a subject identified as having a CBM complex pathway-associated disease or disorder.

A CBM complex pathway-associated disease or disorder can be any appropriate CBM complex pathway-associated disease or disorder, such as any of those described herein. In some embodiments, the CBM complex pathway-associated disease or disorder is an autoimmune disease. In some embodiments, the CBM complex pathway-associated disease or disorder is an inflammatory disease. In some embodiments, the CBM complex pathway-associated cancer is selected from the group consisting of a CBM complex pathway cell surface receptor-associated cancer, a disease or disorder associated with a signal transducer between a cell surface receptor and a CBM complex, a component of a CBM complex-associated cancer, a MALT1 protease substrate-associated cancer, a disease or disorder associated with a component of the NF-κB pathway downstream of a CBM complex, a disease or disorder associated with a component of the JNK pathway downstream of a CBM complex, and a combination thereof. In some embodiments, the CBM complex pathway-associated disease or disorder is a MALT1-associated disease or disorder.

In some cases, compounds of Formula (I), or a pharmaceutically acceptable salt thereof can be useful for inhibiting the processes of cells, such as inhibiting the proliferation of cells. Accordingly, provided herein is a method for inhibiting mammalian cell proliferation, including contacting the mammalian cell with a compound of Formula (I), or a pharmaceutically acceptable salt thereof. Also provided herein is a method for inhibiting CBM complex pathway activity in a mammalian cell, including contacting the mammalian cell with a compound of Formula (I), or a pharmaceutically acceptable salt thereof. Provided also herein is a method for inhibiting MALT1 protease activity in a mammalian cell, including contacting the mammalian cell with a compound of Formula (I), or a pharmaceutically acceptable salt thereof. In some embodiments, the contacting occurs in vivo. In some embodiments, the contacting occurs in vitro. A mammalian cell can be any appropriate cell. In some embodiments, the mammalian cell is a mammalian immune cell. In some embodiments, the mammalian cell is a mammalian cancer cell. In some embodiments, the mammalian cancer cell is a mammalian CBM complex pathway-associated cancer cell. In some embodiments, the mammalian cancer cell is a mammalian MALT1-associated cancer cell. In some embodiments, the mammalian cell has dysregulation of a MALT1 gene, a MALT1 protease protein, or expression or activity or level of any of the same. In some embodiments, the dysregulation of a MALT1 gene, a MALT1 protease protein, or expression or activity or level of any of the same is an IAP2-MALT1 fusion, an IGH-MALT1 fusion, or a combination thereof.

Compounds of Formula (I), or a pharmaceutically acceptable salt thereof can also be useful in the manufacture of medicaments. Accordingly, provided herein is a use of a compound of Formula (I), or a pharmaceutically acceptable salt thereof in the manufacture of a medicament for the treatment of a CBM complex pathway-associated disease or disorder. A CBM complex pathway-associated disease or disorder can be any appropriate CBM complex pathway-associated disease or disorder, such as those described herein. In some embodiments, the CBM complex pathway-associated disease or disorder is selected from the group consisting of a CBM complex pathway cell surface receptor-associated cancer, a disease or disorder associated with a signal transducer between a cell surface receptor and a CBM complex, a component of a CBM complex-associated cancer, a MALT1 protease substrate-associated cancer, a disease or disorder associated with a component of the NF-κB pathway downstream of a CBM complex, a disease or disorder associated with a component of the JNK pathway downstream of a CBM complex, and a combination thereof. In some embodiments, the CBM complex pathway-associated disease or disorder is a CBM complex pathway-associated autoimmune disorder. In some embodiments, the CBM complex pathway-associated disease or disorder is a CBM complex pathway-associated inflammatory disorder. In some embodiments, the CBM complex pathway-associated disease or disorder is a CBM complex pathway-associated cancer. In some embodiments, the CBM complex pathway-associated disease or disorder is a MALT1-associated disease or disorder. In some embodiments, the MALT1-associated disease or disorder comprises a dysregulation of a MALT1 gene, a MALT1 protease protein, or expression or activity or level of any of the same. In some embodiments, the dysregulation of a MALT1 gene, a MALT1 protease protein, or expression or activity or level of any of the same is an IAP2-MALT1 fusion, an IGH-MALT1 fusion, or a combination thereof.

In some embodiments, the compounds provided herein exhibit brain and/or central nervous system (CNS) penetrance. Such compounds are capable of crossing the blood brain barrier and inhibiting a MALT1 protease in the brain and/or other CNS structures. In some embodiments, the compounds provided herein are capable of crossing the blood brain barrier in an effective amount. For example, treatment of a subject with cancer (e.g., a MALT1-associated cancer such as a MALT1-associated brain or CNS cancer) can include administration (e.g., oral administration) of the compound to the subject. In some such embodiments, the compounds provided herein are useful for treating a primary brain tumor or metastatic brain tumor. For example, the compounds can be used in the treatment of one or more of gliomas such as glioblastoma (also known as glioblastoma multiforme), astrocytomas, oligodendrogliomas, ependymomas, and mixed gliomas, meningiomas, medulloblastomas, gangliogliomas, schwannomas (neurilemmomas), and craniopharyngiomas (see, for example, the tumors listed in Louis, D. N. et al. Acta Neuropathol 131(6), 803-820 (June 2016)). In some embodiments, the brain tumor is a primary brain tumor. In some embodiments, the subject has previously been treated with another anticancer agent, e.g., another protease inhibitor (e.g., a compound that is not a compound of Formula (I)). In some embodiments, the brain tumor is a metastatic brain tumor. In some embodiments, the subject has previously been treated with another anticancer agent, e.g., another protease inhibitor (e.g., a compound that is not a compound of Formula (I)).

In some embodiments of any of the methods or uses described herein, an assay used to determine whether the subject has a dysregulation of a gene (e.g., a MALT1 gene), or a protein (e.g., a MALT1 protein), or expression or activity or level of any of the same, using a sample from a subject can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof. Assays can utilize other detection methods known in the art for detecting dysregulation of a gene (e.g., a MALT1 gene), a protein (e.g., a MALT1 protein), or expression or activity or levels of any of the same. In some embodiments, the sample is a biological sample or a biopsy sample (e.g., a paraffin-embedded biopsy sample) from the subject. In some embodiments, the subject is a subject suspected of having a MALT1-associated cancer, a subject having one or more symptoms of a MALT1-associated cancer, and/or a subject that has an increased risk of developing a MALT1-associated cancer).

In some embodiments, dysregulation of a gene (e.g., a MALT1 gene), a MALT1 protein (e.g., a MALT1 protein), or the expression or activity or level of any of the same can be identified using a liquid biopsy (variously referred to as a fluid biopsy or fluid phase biopsy). Liquid biopsy methods can be used to detect total tumor burden and/or the dysregulation of a gene (e.g., a MALT1 protein), a MALT1 protein (e.g., a MALT 1 protein), or the expression or activity or level of any of the same. Liquid biopsies can be performed on biological samples obtained relatively easily from a subject (e.g., via a simple blood draw) and are generally less invasive than traditional methods used to detect tumor burden and/or dysregulation of a gene (e.g., a MALT1 gene), a protein (e.g., a MALT1 protein), or the expression or activity or level of any of the same. In some embodiments, liquid biopsies can be used to detect the presence of dysregulation of a gene (e.g., a MALT1 gene), a protein (e.g., a MALT1 protein), or the expression or activity or level of any of the same at an earlier stage than traditional methods. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of a gene (e.g., a MALT1 gene), a protein (e.g., a MALT1 protein), or the expression or activity or level of any of the same.

In some embodiments, ctDNA derived from a single gene can be detected using a liquid biopsy. In some embodiments, ctDNA derived from a plurality of genes (e.g., 2, 3, 4, 5, 6, 7, 8, 9, 10, 15, 20, 25, 30, 35, 40, 45, 50, 55, 60, 65, 70, 75, 80, 85, 90, 95, 100 or more, or any number of genes in between these numbers) can be detected using a liquid biopsy. In some embodiments, ctDNA derived from a plurality of genes can be detected using any of a variety of commercially-available testing panels (e.g., commercially-available testing panels designed to detect dysregulation of a gene (e.g., a MALT1 gene), a protein (e.g., a MALT1 protein), or the expression or activity or level of any of the same). Liquid biopsies can be used to detect dysregulation of a gene (e.g., a MALT1 gene), a protein (e.g., a MALT1 protein), or the expression or activity or level of any of the same including, without limitation, point mutations or single nucleotide variants (SNVs), copy number variants (CNVs), genetic fusions (e.g., translocations or rearrangements), insertions, deletions, or any combination thereof. In some embodiments, a liquid biopsy can be used to detect a germline mutation. In some embodiments, a liquid biopsy can be used to detect a somatic mutation. In some embodiments, a liquid biopsy can be used to detect a primary genetic mutation (e.g., a primary mutation or a primary fusion that is associated with initial development of a disease, e.g., cancer). In some embodiments, a dysregulation of a gene (e.g., a MALT1 gene), a protein (e.g., a MALT1 protein), or the expression or activity or level of any of the same identified using a liquid biopsy is also present in a cancer cell that is present in the subject (e.g., in a tumor). In some embodiments, any of the types of dysregulation of a gene (e.g., a MALT1 gene), a protein (e.g., a MALT1 protein), or the expression or activity or level of any of the same described herein can be detected using a liquid biopsy. In some embodiments, a genetic mutation identified via a liquid biopsy can be used to identify the subject as a candidate for a particular treatment. For example, detection of dysregulation of a gene (e.g., a MALT1 gene), a protein (e.g., a MALT1 protein), or the expression or activity or level of any of the same in the subject can indicate that the subject will be responsive to a treatment that includes administration of a compound of Formula (I), or a pharmaceutically acceptable salt thereof.

Liquid biopsies can be performed at multiple times during a course of diagnosis, a course of monitoring, and/or a course of treatment to determine one or more clinically relevant parameters including, without limitation, progression of the disease and/or efficacy of a treatment. For example, a first liquid biopsy can be performed at a first time point and a second liquid biopsy can be performed at a second time point during a course of diagnosis, a course of monitoring, and/or a course of treatment. In some embodiments, the first time point can be a time point prior to diagnosing a subject with a disease (e.g., when the subject is healthy), and the second time point can be a time point after subject has developed the disease (e.g., the second time point can be used to diagnose the subject with the disease). In some embodiments, the first time point can be a time point prior to diagnosing a subject with a disease (e.g., when the subject is healthy), after which the subject is monitored, and the second time point can be a time point after monitoring the subject. In some embodiments, the first time point can be a time point after diagnosing a subject with a disease, after which a treatment is administered to the subject, and the second time point can be a time point after the treatment is administered; in such cases, the second time point can be used to assess the efficacy of the treatment (e.g., if the genetic mutation(s) detected at the first time point are reduced in abundance or are undetectable). In some embodiments, a treatment to be administered to a subject can include a compound of Formula (I), or a pharmaceutically acceptable salt thereof.

In some embodiments, the efficacy of a compound of Formula (I), or a pharmaceutically acceptable salt thereof, can be determined by assessing the allele frequency of a dysregulation of a gene (e.g., a MALT1 gene) in cfDNA obtained from a subject at different time points, e.g., cfDNA obtained from the subject at a first time point and cfDNA obtained from the subject at a second time point, where at least one dose of a compound of Formula (I), or a pharmaceutically acceptable salt thereof, is administered to the subject between the first and second time points. Some embodiments of these methods can further include administering to the subject at least one dose of the compound of Formula (I), or a pharmaceutically acceptable salt thereof, between the first and second time points. For example, a reduction (e.g., a 1% to about a 99% reduction, a 1% to about a 95% reduction, a 1% to about a 90% reduction, a 1% to about a 85% reduction, a 1% to about a 80% reduction, a 1% to about a 75% reduction, a 1% reduction to about a 70% reduction, a 1% reduction to about a 65% reduction, a 1% reduction to about a 60% reduction, a 1% reduction to about a 55% reduction, a 1% reduction to about a 50% reduction, a 1% reduction to about a 45% reduction, a 1% reduction to about a40% reduction, a 1% reduction to about a35% reduction, a 1% reduction to about a 30% reduction, a 1% reduction to about a 25% reduction, a 1% reduction to about a 20% reduction, a 1% reduction to about a 15% reduction, a 1% reduction to about a 10% reduction, a 1% to about a 5% reduction, about a 5% to about a 99% reduction, about a 10% to about a 99% reduction, about a 15% to about a 99% reduction, about a 20% to about a 99% reduction, about a 25% to about a 99% reduction, about a 30% to about a 99% reduction, about a 35% to about a 99% reduction, about a 40% to about a 99% reduction, about a 45% to about a 99% reduction, about a 50% to about a 99% reduction, about a 55% to about a 99% reduction, about a 60% to about a 99% reduction, about a 65% to about a 99% reduction, about a 70% to about a 99% reduction, about a 75% to about a 95% reduction, about a 80% to about a 99% reduction, about a 90% reduction to about a 99% reduction, about a 95% to about a 99% reduction, about a 5% to about a 10% reduction, about a 5% to about a 25% reduction, about a 10% to about a 30% reduction, about a 20% to about a 40% reduction, about a 25% to about a 50% reduction, about a 35% to about a 55% reduction, about a 40% to about a 60% reduction, about a 50% reduction to about a 75% reduction, about a 60% reduction to about 80% reduction, or about a 65% to about a 85% reduction) in the allele frequency (AF) of the dysregulation of a gene (e.g., MALT1 gene) in the cfDNA obtained from the subject at the second time point as compared to the allele frequency (AF) of the dysregulation of a gene (e.g., MALT1 gene) in the cfDNA obtained from the subject at the first time point indicates that the compound of Formula (I), or a pharmaceutically acceptable salt thereof, was effective in the subject. In some embodiments, the AF is reduced such that the level is below the detection limit of the instrument. Alternatively, an increase in the allele frequency (AF) of the dysregulation of a gene (e.g., MALT1 gene) in the cfDNA obtained from the subject at the second time point as compared to the allele frequency (AF) of the dysregulation of a gene (e.g., MALT1 gene) in the cfDNA obtained from the subject at the first time point indicates that the compound of Formula (I), or a pharmaceutically acceptable salt thereof, was not effective in the subject. Some embodiments of these methods can further include, administering additional doses of a compound of Formula (I), or a pharmaceutically acceptable salt thereof, to a subject in which a compound of Formula (I), or a pharmaceutically acceptable salt thereof, was determined to be effective. Some embodiments of these methods can further include, administering a different treatment (e.g., a treatment that does not include the administration of a compound of Formula (I), or a pharmaceutically acceptable salt thereof, as a monotherapy) to a subject in which a compound of Formula (I), or a pharmaceutically acceptable salt thereof, was determined not to be effective.

In some examples of these methods, the time difference between the first and second time points can be about 1 day to about 1 year, about 1 day to about 11 months, about 1 day to about 10 months, about 1 day to about 9 months, about 1 day to about 8 months, about 1 day to about 7 months, about 1 day to about 6 months, about 1 day to about 5 months, about 1 day to about 4 months, about 1 day to about 3 months, about 1 day to about 10 weeks, about 1 day to about 2 months, about 1 day to about 6 weeks, about 1 day to about 1 month, about 1 day to about 25 days, about 1 day to about 20 days, about 1 day to about 15 days, about 1 day to about 10 days, about 1 day to about 5 days, about 2 days to about 1 year, about 5 days to about 1 year, about 10 days to about 1 year, about 15 days to about 1 year, about 20 days to about 1 year, about 25 days to about 1 year, about 1 month to about 1 year, about 6 weeks to about 1 year, about 2 months to about 1 year, about 3 months to about 1 year, about 4 months to about 1 year, about 5 months to about 1 year, about 6 months to about 1 year, about 7 months to about 1 year, about 8 months to about 1 year, about 9 months to about 1 year, about 10 months to about 1 year, about 11 months to about 1 year, about 1 day to about 7 days, about 1 day to about 14 days, about 5 days to about 10 days, about 5 day to about 20 days, about 10 days to about 20 days, about 15 days to about 1 month, about 15 days to about 2 months, about 1 week to about 1 month, about 2 weeks to about 1 month, about 1 month to about 3 months, about 3 months to about 6 months, about 4 months to about 6 months, about 5 months to about 8 months, or about 7 months to about 9 months. In some embodiments of these methods, the subject can be previously identified as having a cancer having a dysregulated gene (e.g., any of the examples of a dysregulated gene described herein) (e.g., a MALT1 gene). In some embodiments of these methods, a subject can have been previously diagnosed as having any of the types of cancer described herein. In some embodiments of these methods, the subject can have one or more metastases (e.g., one or more brain metastases).

In some of the above embodiments, the cfDNA comprises ctDNA such as MALT1-associated ctDNA. For example, the cfDNA is ctDNA such as MALT1-associated ctDNA. In some embodiments, at least some portion of cfDNA is determined to be MALT1-associated ctDNA, for example, a sequenced and/or quantified amount of the total cfDNA is determined to have a MALT1 fusion and/or overexpression of MALT1.

In the field of medical oncology, it is normal practice to use a combination of different forms of treatment to treat each subject with cancer. In medical oncology the other component(s) of such conjoint treatment or therapy in addition to compositions provided herein may be, for example, surgery, radiotherapy, and chemotherapeutic agents, such as other protease inhibitors, kinase inhibitors, signal transduction inhibitors, and/or monoclonal antibodies.

For example, a surgery may be open surgery or minimally invasive surgery. Compounds of Formula (I), or a pharmaceutically acceptable salt thereof therefore may also be useful as adjuvants to cancer treatment, that is, they can be used in combination with one or more additional therapies or therapeutic agents, for example, a chemotherapeutic agent that works by the same or by a different mechanism of action. In some embodiments, a compound of Formula (I), or a pharmaceutically acceptable salt thereof, can be used prior to administration of an additional therapeutic agent or additional therapy. For example, a subject in need thereof can be administered one or more doses of a compound of Formula (I), or a pharmaceutically acceptable salt thereof for a period of time and then undergo at least partial resection of the tumor. In some embodiments, the treatment with one or more doses of a compound of Formula (I), or a pharmaceutically acceptable salt thereof reduces the size of the tumor (e.g., the tumor burden) prior to the at least partial resection of the tumor. In some embodiments, a subject in need thereof can be administered one or more doses of a compound of Formula (I), or a pharmaceutically acceptable salt thereof for a period of time and under one or more rounds of radiation therapy. In some embodiments, the treatment with one or more doses of a compound of Formula (I), or a pharmaceutically acceptable salt thereof reduces the size of the tumor (e.g., the tumor burden) prior to the one or more rounds of radiation therapy.

In some embodiments, a subject has a cancer (e.g., a locally advanced or metastatic tumor) that is refractory or intolerant to standard therapy (e.g., administration of a chemotherapeutic agent), such as a first MALT1 inhibitor, a kinase inhibitor, immunotherapy, cell or gene therapy, or radiation (e.g., radioactive iodine). In some embodiments, a subject has a cancer (e.g., a locally advanced or metastatic tumor) that is refractory or intolerant to prior therapy (e.g., administration of a chemotherapeutic agent, such as a first MALT1 inhibitor or another protease inhibitor, immunotherapy, cell or gene therapy, or radiation (e.g., radioactive iodine). In some embodiments, a subject has a cancer (e.g., a locally advanced or metastatic tumor) that has no standard therapy. In some embodiments, a subject is MALT1-protease inhibitor naïve. For example, the subject is naïve to treatment with a selective MALT1-protease inhibitor. In some embodiments, a subject is not MALT1-protease inhibitor naïve.

In some embodiments of any of the methods described herein, the compound of Formula (I), or a pharmaceutically acceptable salt thereof, is administered in combination with an effective amount of at least one additional therapeutic agent selected from one or more additional therapies or therapeutic (e.g., chemotherapeutic or immunomodulatory) agents. An additional therapy or therapeutic agent can be any appropriate additional therapy or therapeutic agent, such as any of those described herein.

Non-limiting examples of additional therapeutic agents include: other MALT1-targeted therapeutic agents (i.e. a first or second MALT1 protease inhibitor, e.g., JNJ-67856633 or CTX-177), other protease inhibitors, kinase inhibitors (e.g., receptor tyrosine kinase-targeted therapeutic agents such as BTK or EGFR inhibitors), signal transduction pathway inhibitors, checkpoint inhibitors, modulators of the apoptosis pathway (e.g., venetoclax or obataclax); cytotoxic chemotherapeutics, angiogenesis-targeted therapies, immune-targeted agents (including antibody and cell-based immunotherapies, and antibody-drug conjugates) and radiotherapy.

In some embodiments, the compound of Formula (I), or a pharmaceutically acceptable salt thereof, and the additional therapeutic agent are administered simultaneously as separate dosages. In some embodiments, the compound of Formula (I), or a pharmaceutically acceptable salt thereof, and the additional therapeutic agent are administered as separate dosages sequentially in any order.

In some embodiments, the other MALT1-targeted therapeutic is another protease inhibitor exhibiting MALT1 inhibition activity. In some embodiments, the other MALT1-targeted therapeutic inhibitor is selective for a MALT1 protease. Exemplary MALT1 protease inhibitors can exhibit inhibition activity (IC₅₀) against a MALT1 protease of less than about 1000 nM, less than about 500 nM, less than about 200 nM, less than about 100 nM, less than about 50 nM, less than about 25 nM, less than about 10 nM, or less than about 1 nM as measured in an assay as described herein. In some embodiments, a MALT1 protease inhibitors can exhibit inhibition activity (IC₅₀) against a MALT1 protease of less than about 25 nM, less than about 10 nM, less than about 5 nM, or less than about 1 nM as measured in an assay as provided herein.

Non-limiting examples of protease-targeted therapeutic agents (e.g., a first MALT1 inhibitor or a second MALT1 inhibitor) include JNJ-67856633 and CTX-177.

Non-limiting examples of multi-kinase inhibitors include alectinib (9-Ethyl-6,6-dimethyl-8-[4-(morpholin-4-yl)piperidin-1-yl]-11-oxo-6,11-dihydro-5H-benzo[b]carbazole-3-carbonitrile); amuvatinib (MP470, HPK56) (N-(1,3-benzodioxol-5-ylmethyl)-4-([1]benzofuro[3,2-d]pyrimidin-4-yl)piperazine-1-carbothioamide); apatinib (YN968D1) (N-[4-(1-cyanocyclopentyl) phenyl-2-(4-picolyl)amino-3-Nicotinamide methanesulphonate); cabozantinib (Cometriq XL-184) (N-(4-((6,7-Dimethoxyquinolin-4-yl)oxy)phenyl)-N′-(4-fluorophenyl)cyclopropane-1,1-dicarboxamide); dovitinib (TK1258; GFKI-258; CHIR-258) ((3Z)-4-amino-5-fluoro-3-[5-(4-methylpiperazin-1-yl)-1,3-dihydrobenzimidazol-2-ylidene]quinolin-2-one); famitinib (5-[2-(diethylamino)ethyl]-2-[(Z)-(5-fluoro-2-oxo-1H-indol-3-ylidene)methyl]-3-methyl-6,7-dihydro-1H-pyrrolo[3,2-c]pyridin-4-one); fedratinib (SAR302503, TG101348) (N-(2-Methyl-2-propanyl)-3-{[5-methyl-2-({4-[2-(1-pyrrolidinyl)ethoxy]phenyl}amino)-4-pyrimidinyl]amino}benzenesulfonamide); foretinib (XL880, EXEL-2880, GSK1363089, GSK089) (N1′-[3-fluoro-4-[[6-methoxy-7-(3-morpholinopropoxy)-4-quinolyl]oxy]phenyl]-N1-(4-fluorophenyl)cyclopropane-1,1-dicarboxamide); fostamantinib (R788) (2H-Pyrido[3,2-b]-1,4-oxazin-3(4H)-one, 6-[[5-fluoro-2-[(3,4,5-trimethoxyphenyl)amino]-4-pyrimidinyl]amino]-2,2-dimethyl-4-[(phosphonooxy)methyl]-, sodium salt (1:2)); ilorasertib (ABT-348) (1-(4-(4-amino-7-(1-(2-hydroxyethyl)-1H-pyrazol-4-yl)thieno[3,2-c]pyridin-3-yl)phenyl)-3-(3-fluorophenyl)urea); lenvatinib (E7080, Lenvima) (4-[3-chloro-4-(cyclopropylaminocarbonyl)aminophenoxy]-7-methoxy-6-quinolinecarboxamide); motesanib (AMG 706) (N-(3,3-Dimethyl-2,3-dihydro-1H-indol-6-yl)-2-[(pyridin-4-ylmethyl)amino]pyridine-3-carboxamide); nintedanib (3-Z-[1-(4-(N-((4-methyl-piperazin-1-yl)-methylcarbonyl)-N-methyl-amino)-anilino)-1-phenyl-methylene]-6-methyoxycarbonyl-2-indolinone); ponatinib (AP24534) (3-(2-Imidazo[1,2-b]pyridazin-3-ylethynyl)-4-methyl-N-[4-[(4-methylpiperazin-1-yl)methyl]-3-(trifluoromethyl)phenyl]benzamide); PP242 (torkinib) (2-[4-Amino-1-(1-methylethyl)-1H-pyrazolo[3,4-d]pyrimidin-3-yl]-1H-indol-5-ol); quizartinib (1-(5-(tert-Butyl)isoxazol-3-yl)-3-(4-(7-(2-morpholinoethoxy)benzo[d]imidazo[2,1-b]thiazol-2-yl)phenyl)urea); regorafenib (BAY 73-4506, stivarga) (4-[4-({[4-Chloro-3-(trifluoromethyl)phenyl]carbamoyl}amino)-3-fluorophenoxy]-N-methylpyridine-2-carboxamide hydrate); RXDX-105 (CEP-32496, agerafenib) (1-(3-((6,7-dimethoxyquinazolin-4-yl)oxy)phenyl)-3-(5-(1,1,1-trifluoro-2-methylpropan-2-yl)isoxazol-3-yl)urea); semaxanib (SU5416) ((3Z)-3-[(3,5-dimethyl-1H-pyrrol-2-yl)methylidene]-1,3-dihydro-2H-indol-2-one); sitravatinib (MGCD516, MG516) (N-(3-Fluoro-4-{[2-(5-{[(2-methoxyethyl)amino]methyl}-2-pyridinyl)thieno[3,2-b]pyridin-7-yl]oxy}phenyl)-N′-(4-fluorophenyl)-1,1-cyclopropanedicarboxamide); sorafenib (BAY 43-9006) (4-[4-[[[[4-chloro-3-(trifluoromethyl)phenyl]amino]carbonyl]amino]phenoxy]-N-methyl-2-pyridinecarboxamide); vandetanib (N-(4-bromo-2-fluorophenyl)-6-methoxy-7-[(1-methylpiperidin-4-yl)methoxy]quinazolin-4-amine); vatalanib (PTK787, PTK/ZK, ZK222584) (N-(4-chlorophenyl)-4-(pyridin-4-ylmethyl)phthalazin-1-amine); AD-57 (N-[4-[4-amino-1-(1-methylethyl)-1H-pyrazolo[3,4-d]pyrimidin-3-yl]phenyl]-N′-[3-(trifluoromethyl)phenyl]-urea); AD-80 (1-[4-(4-amino-1-propan-2-ylpyrazolo[3,4-d]pyrimidin-3-yl)phenyl]-3-[2-fluoro-5-(trifluoromethyl)phenyl]urea); AD-81 (1-(4-(4-amino-1-isopropyl-1H-pyrazolo[3,4-d]pyrimidin-3-yl)phenyl)-3-(4-chloro-3-(trifluoromethyl)phenyl)urea); ALW-II-41-27 (N-(5-((4-((4-ethylpiperazin-1-yl)methyl)-3-(trifluoromethyl)phenyl)carbamoyl)-2-methylphenyl)-5-(thiophen-2-yl)nicotinamide); BPR1K871 (1-(3-chlorophenyl)-3-(5-(2-((7-(3-(dimethylamino)propoxy)quinazolin-4-yl)amino)ethyl)thiazol-2-yl)urea); CLM3 (1-phenethyl-N-(1-phenylethyl)-1H-pyrazolo[3,4-d]pyrimidin-4-amine); EBI-907 (N-(2-chloro-3-(1-cyclopropyl-8-methoxy-3H-pyrazolo[3,4-c]isoquinolin-7-yl)-4-fluorophenyl)-3-fluoropropane-1-sulfonamide); NVP-AST-487 (N-[4-[(4-ethyl-1-piperazinyl)methyl]-3-(trifluoromethyl)phenyl]-N′-[4-[[6-(methylamino)-4-pyrimidinyl]oxy]phenyl]-urea); NVP-BBT594 (BBT594) (5-((6-acetamidopyrimidin-4-yl)oxy)-N-(4-((4-methylpiperazin-1-yl)methyl)-3-(trifluoromethyl)phenyl) indoline-1-carboxamide); PD173955 (6-(2,6-dichlorophenyl)-8-methyl-2-(3-methylsulfanylanilino)pyrido[2,3-d]pyrimidin-7-one); PP2 (4-amino-5-(4-chlorophenyl)-7-(dimethylethyl)pyrazolo[3,4-d]pyrimidine); PZ-1 (N-(5-(tert-butyl)isoxazol-3-yl)-2-(4-(5-(1-methyl-1H-pyrazol-4-yl)-1Hbenzo[d]imidazol-1-yl)phenyl)acetamide); RPI-1 (1,3-dihydro-5,6-dimethoxy-3-[(4-hydroxyphenyl)methylene]-H-indol-2-one; (3E)-3-[(4-hydroxyphenyl)methylidene]-5,6-dimethoxy-1H-indol-2-one); SGI-7079 (3-[2-[[3-fluoro-4-(4-methyl-1-piperazinyl)phenyl]amino]-5-methyl-7H-pyrrolo[2,3-d]pyrimidin-4-yl]-benzeneacetonitrile); SPP86 (1-Isopropyl-3-(phenylethynyl)-1H-pyrazolo[3,4-d]pyrimidin-4-amine); SU4984 (4-[4-[(E)-(2-oxo-1H-indol-3-ylidene)methyl]phenyl]piperazine-1-carbaldehyde); sunitinb (SU11248) (N-(2-Diethylaminoethyl)-5-[(Z)-(5-fluoro-2-oxo-1H-indol-3-ylidene)methyl]-2,4-dimethyl-1H-pyrrole-3-carboxamide); TG101209 (N-tert-butyl-3-(5-methyl-2-(4-(4-methylpiperazin-1-yl)phenylamino)pyrimidin-4-ylamino)benzenesulfonamide); Withaferin A ((4β,5β,6β,22R)-4,27-Dihydroxy-5,6:22,26-diepoxyergosta-2,24-diene-1,26-dione); XL-999 ((Z)-5-((1-ethylpiperidin-4-yl)amino)-3-((3-fluorophenyl)(5-methyl-1H-imidazol-2-yl)methylene) indolin-2-one); BPR1J373 (a 5-phenylthiazol-2-ylamine-pyriminide derivative); CG-806 (CG'806); DCC-2157; GTX-186; HG-6-63-01 ((E)-3-(2-(4-chloro-1H-pyrrolo[2,3-b]pyridin-5-yl)vinyl)-N-(4-((4-ethylpiperazin-1-yl)methyl)-3-(trifluoromethyl)phenyl)-4-methylbenzamide); SW-01 (Cyclobenzaprine hydrochloride); XMD15-44 (N-(4-((4-ethylpiperazin-1-yl)methyl)-3-(trifluoromethyl)phenyl)-4-methyl-3-(pyridin-3-ylethynyl)benzamide (generated from structure)); ITRI-305 (DON5 TB, DIB003599); BLU-667 ((1S,4R)—N—((S)-1-(6-(4-fluoro-1H-pyrazol-1-yl)pyridin-3-yl)ethyl)-1-methoxy-4-(4-methyl-6-((5-methyl-1H-pyrazol-3-yl)amino)pyrimidin-2-yl)cyclohexane-1-carboxamide); BLU6864; DS-5010; GSK3179106; GSK3352589; NMS-E668; TAS0286/HM05; TPX0046; and N-(3-(2-(dimethylamino)ethoxy)-5-(trifluoromethyl)phenyl)-2-(4-(4-ethoxy-6-oxo-1,6-dihydropyridin-3-yl)-2-fluorophenyl)acetamide.

Non-limiting examples of receptor tyrosine kinase (e.g., Trk) targeted therapeutic agents, include afatinib, cabozantinib, cetuximab, crizotinib, dabrafenib, entrectinib, erlotinib, gefitinib, imatinib, lapatinib, lestaurtinib, nilotinib, pazopanib, panitumumab, pertuzumab, sunitinib, trastuzumab, 1-((3S,4R)-4-(3-fluorophenyl)-1-(2-methoxyethyl)pyrrolidin-3-yl)-3-(4-methyl-3-(2-methylpyrimidin-5-yl)-1-phenyl-1H-pyrazol-5-yl)urea, AG 879, AR-772, AR-786, AR-256, AR-618, AZ-23, AZ623, DS-6051, G6 6976, GNF-5837, GTx-186, GW 441756, LOXO-101, MGCD516, PLX7486, RXDX101, VM-902A, TPX-0005, TSR-Oll, GNF-4256, N-[3-[[2,3-dihydro-2-oxo-3-(1H-pyrrol-2-ylmethylene)-1H-indol-6-yl]amino]-4-methylphenyl]-N′-[2-fluoro-5-(trifluoromethyl)phenyl]-urea, AZ623, AZ64, (S)-5-Chloro-N2-(1-(5-fluoropyridin-2-yl)ethyl)-N4-(5-isopropoxy-1H-pyrazol-3-yl)pyrimidine-2,4-diamine, AZD7451, CEP-751, CT327, sunitinib, GNF-8625, and (R)-1-(6-(6-(2-(3-fluorophenyl)pyrrolidin-1-yl)imidazo[1,2-b]pyridazin-3-yl)-[2,4′-bipyridin]-2′-yl)piperidin-4-ol.

In some embodiments, the additional therapeutic agent is a BRAF inhibitor. Non-limiting examples of a BRAF inhibitor include dabrafenib, vemurafenib (also called R^{G}7204 or PLX4032), sorafenib tosylate, PLX-4720, GDC-0879, BMS-908662 (Bristol-Meyers Squibb), LGX818 (Novartis), PLX3603 (Hofmann-LaRoche), RAF265 (Novartis), RO5185426 (Hofmann-LaRoche), and GSK2118436 (GlaxoSmithKline). Additional examples of a BRAF inhibitor are known in the art.

In some embodiments, the additional therapeutic agent is an epidermal growth factor receptor typrosine kinase inhibitor (EGFR). For example, EGFR inhibitors can include osimertinib (merelectinib, Tagrisso), erlotinib (Tarceva), gefitinib (Iressa), cetuximab (Erbitux), necitumumab (Portrazza), neratinib (Nerlynx), lapatinib (Tykerb), panitumumab (Vectibix), and vandetanib (Caprelsa).

In some embodiments, the additional therapeutic agent is a Ras-Raf-MEK-ERK pathway inhibitors (e.g., binimetinib, selumetinib, encorafenib, sorafenib, trametinib, and vemurafenib), PI3K-Akt-mTOR-S6K pathway inhibitors (e.g., everolimus, rapamycin, perifosine, temsirolimus), and other kinase inhibitors, such as baricitinib, brigatinib, capmatinib, danusertib, ibrutinib, milciclib, quercetin, regorafenib, ruxolitinib, semaxanib, AP32788, BLU285, BLU554, INCB39110, INCB40093, INCB50465, INCB52793, INCB54828, MGCD265, NMS-088, NMS-1286937, PF 477736 ((R)-amino-N-[5,6-dihydro-2-(1-methyl-1H-pyrazol-4-yl)-6-oxo-1Hpyrrolo[4,3,2-ef][2,3]benzodiazepin-8-yl]-cyclohexaneacetamide), PLX3397, PLX7486, PLX8394, PLX9486, PRN1008, PRN1371, RXDX103, RXDX106, RXDX108, and TG101209 (N-tert-butyl-3-(5-methyl-2-(4-(4-methylpiperazin-1-yl)phenylamino)pyrimidin-4-ylamino)benzenesulfonamide).

In some embodiments, the additional therapeutic agent is a BTK inhibitor. Non-limiting examples of BTK inhibitors include ibrutinib, acalabrutinib, and zanubrutinib.

In some embodiments, the additional therapeutic agent is a Bcl-2 inhibitor. Non-limiting examples of Bcl-2 inhibitors include venetoclax, navitoclax, oblimersen, obatoclax, and AT-101.

In some embodiments, the additional therapeutic agent is a PI3K inhibitor. Non-limiting examples of PI3K inhibitors include idelalisib, copanlisib, duvelisib, alpelisib, taselisib, buparlisib, umbralisib, and copanlisib.

In some embodiments, the additional therapeutic agent is a mTOR inhibitor. Non-limiting examples of mTOR inhibitors include everolimus, temsirolimus, and ridaforolimus.

In some embodiments, the additional therapeutic agent is a HDAC inhibitor. Non-limiting examples of HDAC inhibitors include vorinostat, romidepsin, belinostat, chidamide, panobinostat, CXD101, and abexinostat.

In some embodiments, the additional therapeutic agent is a checkpoint inhibitor. Non-limiting examples of checkpoint inhibitors include ipilimumab, tremelimumab, nivolumab, pidilizumab, MPDL3208A, MEDI4736, MSB0010718C, BMS-936559, BMS-956559, BMS-935559 (MDX-1105), AMP-224, and pembrolizumab.

In some embodiments, the additional therapeutic agent is a cytotoxic chemotherapeutic. Non-limiting example of cytotoxic chemotherapeutics include arsenic trioxide, bleomycin, bendamustine, cabazitaxel, capecitabine, carboplatin, cisplatin, cyclophosphamide, cytarabine, dacarbazine, daunorubicin, docetaxel, doxorubicin, etoposide, fluorouracil, gemcitabine, irinotecan, lomustine, methotrexate, mitomycin C, oxaliplatin, paclitaxel, pemetrexed, temozolomide, and vincristine.

In some embodiments, the additional therapeutic agent is an angiogenesis-targeted therapeutic. Non-limiting examples of angiogenesis-targeted therapies include lenalidomide, enzastaurine, aflibercept, and bevacizumab.

In some embodiments, an additional therapy or therapeutic agent can include a histidyl-tRNA synthetase (HRS) polypeptide or an expressible nucleotide that encodes the HRS polypeptide.

The term “immunotherapy” refers to an agent that modulates the immune system. In some embodiments, an immunotherapy can increase the expression and/or activity of a regulator of the immune system. In some embodiments, an immunotherapy can decrease the expression and/or activity of a regulator of the immune system. In some embodiments, an immunotherapy can recruit and/or enhance the activity of an immune cell.

In some embodiments, the immunotherapy is a cellular immunotherapy (e.g., adoptive T-cell therapy, dendritic cell therapy, natural killer cell therapy). In some embodiments, the cellular immunotherapy is sipuleucel-T (APC8015; Provenge™; Plosker (2011) Drugs 71(1): 101-108). In some embodiments, the cellular immunotherapy includes cells that express a chimeric antigen receptor (CAR). In some embodiments, the cellular immunotherapy is a CAR-T cell therapy. In some embodiments, the CAR-T cell therapy is tisagenlecleucel (Kymria). In some embodiments, the CAR-T cell therapy is axicabtagene ciloleucel (Yescarta). In some embodiments, the CAR-T cell therapy is brexucabtagene autoleucel (Tecartus). In some embodiments, the CAR-T cell therapy is relmacabtagene autoleucel. In some embodiments, the CAR-T cell therapy is ALLO-501.

In some embodiments, the immunotherapy is an antibody therapy (e.g., a monoclonal antibody, a conjugated antibody, or a bispecific antibody). In some embodiments, the antibody therapy is bevacizumab (Mvasti™, Avastin®), trastuzumab (Herceptin®), avelumab (Bavencio®), rituximab (MabTheram™, Rituxan®), rituximab with human hyaluronidase (Rituxan Hycela™), edrecolomab (Panorex), daratumuab (Darzalex®), olaratumab (Lartruvo™), ofatumumab (Arzerra®), alemtuzumab (Campath®), cetuximab (Erbitux®), oregovomab, pembrolizumab (Keytruda®), dinutiximab (Unituxin®), obinutuzumab (Gazyva®), tremelimumab (CP-675,206), ramucirumab (Cyramza®), ublituximab (TG-1101), panitumumab (Vectibix®), elotuzumab (Empliciti™), avelumab (Bavencio®), necitumumab (Portrazzam), cirmtuzumab (UC-961), ibritumomab (Zevalin®), isatuximab (SAR650984), nimotuzumab, fresolimumab (GC1008), lirilumab (INN), mogamulizumab (Poteligeo®), ficlatuzumab (AV-299), denosumab (Xgeva®), lenzilumab, avelumab, spartalizumab, pembrolizumab, utomilumab, ublituximab, blinatumomab ganitumab, urelumab, pidilizumab, amatuximab, mosunetuzumab (BTCT4465A), CD20-TCB, RO7082859, XmAb13676, glofitamab, CD20-TDB, odronextamab (REGN1979), IGM-2323, BTCT4465A, AMG-562, or TTI-621.

In some embodiments, the immunotherapy is an antibody-drug conjugate. In some embodiments, the antibody-drug conjugate is gemtuzumab ozogamicin (Mylotarg™), inotuzumab ozogamicin (Besponsa®), brentuximab vedotin (Adcetris®), ado-trastuzumab emtansine (TDM-1; Kadcyla®), mirvetuximab soravtansine (IMGN853), anetumab ravtansine, polatuzumab vedotine, loncastuximab tesirine (ADCT-402), camidanlumab tesirine (ADCT-301), or naratuximab emtansine (Debio 1562).

In some embodiments, the immunotherapy includes blinatumomab (AMG103; Blincyto®) or midostaurin (Rydapt).

In some embodiments, the immunotherapy includes a toxin. In some embodiments, the immunotherapy is denileukin diftitox (Ontak®).

In some embodiments, the immunotherapy is a cytokine therapy. In some embodiments, the cytokine therapy is an interleukin 2 (IL-2) therapy, an interferon alpha (IFNα) therapy, a granulocyte colony stimulating factor (G-CSF) therapy, an interleukin 12 (IL-12) therapy, an interleukin 15 (IL-15) therapy, an interleukin 7 (IL-7) therapy or an erythropoietin-alpha (EPO) therapy. In some embodiments, the IL-2 therapy is aldesleukin (Proleukin®). In some embodiments, the IFNα therapy is IntronA® (Roferon-A®). In some embodiments, the G-CSF therapy is filgrastim (Neupogen®).

In some embodiments, the immunotherapy is an immune checkpoint inhibitor. In some embodiments, the immunotherapy includes one or more immune checkpoint inhibitors. In some embodiments, the immune checkpoint inhibitor is a CTLA-4 inhibitor, a PD-1 inhibitor or a PD-L1 inhibitor. In some embodiments, the CTLA-4 inhibitor is ipilimumab (Yervoy®) or tremelimumab (CP-675,206). In some embodiments, the PD-1 inhibitor is pembrolizumab (Keytruda®) or nivolumab (Opdivo®). In some embodiments, the PD-L 1 inhibitor is atezolizumab (Tecentriq®), avelumab (Bavencio®) or durvalumab (Imfinzi™).

In some embodiments, the immunotherapy is mRNA-based immunotherapy. In some embodiments, the mRNA-based immunotherapy is CV9104 (see, e.g., Rausch et al. (2014) Human Vaccin Immunother 10(11): 3146-52; and Kubler et al. (2015) J. Immunother Cancer 3:26).

In some embodiments, the immunotherapy is bacillus Calmette-Guerin (BCG) therapy.

In some embodiments, the immunotherapy is an oncolytic virus therapy. In some embodiments, the oncolytic virus therapy is talimogene alherparepvec (T-VEC; Imlygic®).

In some embodiments, the immunotherapy is a cancer vaccine. In some embodiments, the cancer vaccine is a human papillomavirus (HPV) vaccine. In some embodiments, the HPV vaccine is Gardasil®, Gardasil9® or Cervarix®. In some embodiments, the cancer vaccine is a hepatitis B virus (HBV) vaccine. In some embodiments, the HBV vaccine is Engerix-B®, Recombivax HB® or GI-13020 (Tarmogen®). In some embodiments, the cancer vaccine is Twinrix® or Pediarix®. In some embodiments, the cancer vaccine is BiovaxID®, Oncophage®, GVAX, ADXS11-001, ALVAC-CEA, PROSTVAC®, Rindopepimut®, CimaVax-EGF, lapuleucel-T (APC8024; Neuvenge™), GRNVAC1, GRNVAC2, GRN-1201, hepcortespenlisimut-L (Hepko-V5), DCVAX®, SCIB1, BMT CTN 1401, PrCa VBIR, PANVAC, ProstAtak®, DPX-Survivac, or viagenpumatucel-L (HS-110).

In some embodiments, the immunotherapy is a peptide vaccine. In some embodiments, the peptide vaccine is nelipepimut-S (E75)(NeuVax™), IMA901, or SurVaxM (SVN53-67). In some embodiments, the cancer vaccine is an immunogenic personal neoantigen vaccine (see, e.g., Ott et al. (2017) Nature 547: 217-221; Sahin et al. (2017) Nature 547: 222-226). In some embodiments, the cancer vaccine is RGSH4K, or NEO-PV-01. In some embodiments, the cancer vaccine is a DNA-based vaccine. In some embodiments, the DNA-based vaccine is a mammaglobin-A DNA vaccine (see, e.g., Kim et al. (2016) OncoImmunology 5(2): e1069940).

In some embodiments, immune-targeted agents are selected from aldesleukin, interferon alfa-2b, ipilimumab, lambrolizumab, nivolumab, prednisone, and sipuleucel-T.

In some embodiments, the additional therapy is radiotherapy. Non-limiting examples of radiotherapy include radioiodide therapy, extemal-beam radiation, and radium 223 therapy.

In some embodiments, the additional therapeutic agent is GSK-3368715, PF-06821497, ceralasertib; AZD6738, BI-894999, MAK-683, AZD-6738, taminadenant, TAK-981, MIK-665, or danvatirsen.

Additional kinase inhibitors include those described in, for example, U.S. Pat. Nos. 7,514,446; 7,863,289; 8,026,247; 8,501,756; 8,552,002; 8,815,901; 8,912,204; 9,260,437; 9,273,051; U.S. Publication No. US 2015/0018336; International Publication No. WO 2007/002325; WO 2007/002433; WO 2008/080001; WO 2008/079906; WO 2008/079903; WO 2008/079909; WO 2008/080015; WO 2009/007748; WO 2009/012283; WO 2009/143018; WO 2009/143024; WO 2009/014637; 2009/152083; WO 2010/111527; WO 2012/109075; WO 2014/194127; WO 2015/112806; WO 2007/110344; WO 2009/071480; WO 2009/118411; WO 2010/031816; WO 2010/145998; WO 2011/092120; WO 2012/101032; WO 2012/139930; WO 2012/143248; WO 2012/152763; WO 2013/014039; WO 2013/102059; WO 2013/050448; WO 2013/050446; WO 2014/019908; WO 2014/072220; WO 2014/184069; WO 2016/075224; WO 2016/081450; WO 2016/022569; WO 2016/011141; WO 2016/011144; WO 2016/011147; WO 2015/191667; WO 2012/101029; WO 2012/113774; WO 2015/191666; WO 2015/161277; WO 2015/161274; WO 2015/108992; WO 2015/061572; WO 2015/058129; WO 2015/057873; WO 2015/017528; WO/2015/017533; WO 2014/160521; and WO 2014/011900, each of which is hereby incorporated by reference in its entirety.

In some embodiments, the subject was previously administered one or more standard of care therapies for a lymphoma. In some embodiments, the previously administered standard of care therapy is polatuzumab vedotine, selinexor, axicabtagene ciloleucel (Yescarta), tisagenlecleucel (Kymriah), bendamustine in combination with rituximab and polatuzumab vedotin, tafasitamab in combination with lenalidomide, or rituximab with human hyaluronidase (Rituxan Hycela).

In some embodiments, the subject is concomitantly receiving standard of care therapy for a lymphoma. In some embodiments, the standard of care therapy is polatuzumab vedotine, selinexor, axicabtagene ciloleucel (Yescarta), tisagenlecleucel (Kymriah), bendamustine in combination with rituximab and polatuzumab vedotin, tafasitamab in combination with lenalidomide, or rituximab with human hyaluronidase (Rituxan Hycela).

Although the genetic basis of tumorigenesis may vary between different cancer types, the cellular and molecular mechanisms required for metastasis appear to be similar for all solid tumor types. During a metastatic cascade, the cancer cells lose growth inhibitory responses, undergo alterations in adhesiveness and produce enzymes that can degrade extracellular matrix components. This leads to detachment of tumor cells from the original tumor, infiltration into the circulation through newly formed vasculature, migration and extravasation of the tumor cells at favorable distant sites where they may form colonies.

Accordingly, also provided herein are methods for inhibiting, preventing, aiding in the prevention, or decreasing the symptoms of metastasis of a cancer in a subject in need thereof, the method comprising administering to the subject an effective amount of a compound of Formula (I), or a pharmaceutically acceptable salt thereof or a pharmaceutical composition thereof. Such methods can be used in the treatment of one or more of the cancers described herein. See. e.g., US Publication No. 2013/0029925; International Publication No. WO 2014/083567; and U.S. Pat. No. 8,568,998. See also, e.g., Hezam K et al., Rev Neurosci 2018 Jan. 26; 29:93-98; Gao L, et al., Pancreas 2015 January; 44:134-143; Ding K et al., J Biol Chem 2014 Jun. 6; 289:16057-71; and Amit M et al., Oncogene 2017 Jun. 8; 36:3232-3239. In some embodiments, the cancer is a MALT1-associated cancer. In some embodiments, the compound of Formula (I), or a pharmaceutically acceptable salt thereof is used in combination with an additional therapy or another therapeutic agent, as described herein. For example, a first or second MALT1 protease inhibitor.

The term “metastasis” is an art known term and means the formation of an additional tumor (e.g., a solid tumor) at a site distant from a primary tumor in a subject, where the additional tumor includes the same or similar cancer cells as the primary tumor.

Also provided are methods of decreasing the risk of developing a metastasis or an additional metastasis in a subject having a MALT1-associated cancer that include: selecting, identifying, or diagnosing a subject as having a MALT1-associated cancer, and administering an effective amount of a compound of Formula (I), or a pharmaceutically acceptable salt thereof to the subject selected, identified, or diagnosed as having a MALT1-associated cancer. Also provided are methods of decreasing the risk of developing a metastasis or an additional metastasis in a subject having a MALT1-associated cancer that includes administering an effective amount of a compound of Formula (I), or a pharmaceutically acceptable salt thereof to a subject having a MALT1-associated cancer. The decrease in the risk of developing a metastasis or an additional metastasis in a subject having a MALT1-associated cancer can be compared to the risk of developing a metastasis or an additional metastasis in the subject prior to treatment, or as compared to a subject or a population of subjects having a similar or the same MALT1-associated cancer that has received no treatment or a different treatment.

The phrase “risk of developing a metastasis” means the risk that a subject having a primary tumor will develop an additional tumor (e.g., a solid tumor) at a site distant from a primary tumor in a subject over a set period of time, where the additional tumor includes the same or similar cancer cells as the primary tumor. Methods for reducing the risk of developing a metastasis in a subject having a cancer are described herein.

The phrase “risk of developing additional metastases” means the risk that a subject having a primary tumor and one or more additional tumors at sites distant from the primary tumor (where the one or more additional tumors include the same or similar cancer cells as the primary tumor) will develop one or more further tumors distant from the primary tumor, where the further tumors include the same or similar cancer cells as the primary tumor. Methods for reducing the risk of developing additional metastasis are described herein.

Some embodiments described herein provide methods of treating an autoimmune disorder (e.g., a MALT1-associated autoimmune disorder), such as rheumatoid arthritis, multiple sclerosis, and SLE, the method comprising administering an effective amount of a compound of Formula (I), or a pharmaceutically acceptable salt thereof, to a subject in need thereof.

Some embodiments described herein provide methods of treating an inflammatory disorder (e.g., a MALT1-associated autoimmune disorder), such as chronic graft versus host disease, the method comprising administering an effective amount of a compound of Formula (I), or a pharmaceutically acceptable salt thereof, to a subject in need thereof.

Also provided is a method for inhibiting MALT1 protease activity in a mammalian cell, comprising contacting the mammalian cell with a compound of Formula (I). In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof to a subject having a mammalian cell having MALT1 protease activity. In some embodiments, the mammalian cell is a mammalian immune cell. In some embodiments, the mammalian cell is a mammalian cancer cell. In some embodiments, the mammalian cancer cell is any cancer as described herein. In some embodiments, the mammalian cancer cell is a MALT1-associated mammalian cancer cell.

Also provided is a method for inhibiting MALT1 protease activity in a mammalian mammalian cell, comprising contacting the mammalian cell with a compound of Formula (I). In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof to a mammal having a mammalian cell having MALT1 protease activity. In some embodiments, the mammalian cell is a mammalian immune cell. In some embodiments, the mammalian cell is a mammalian cancer cell. In some embodiments, the mammalian cancer cell is any cancer as described herein. In some embodiments, the mammalian cancer cell is a MALT1-associated mammalian cancer cell. In some embodiments, the mammalian cell is a gastrointestinal mammalian cell.

As used herein, the term “contacting” refers to the bringing together of indicated moieties in an in vitro system or an in vivo system. For example, “contacting” a MALT1 protease with a compound provided herein includes the administration of a compound provided herein to a subject, such as a human, having a MALT1 protease, as well as, for example, introducing a compound provided herein into a sample containing a mammalian cellular or purified preparation containing the MALT1 protease.

Also provided herein is a method of inhibiting mammalian cell proliferation, in vitro or in vivo, the method comprising contacting a mammalian cell with an effective amount of a compound of Formula (I), or a pharmaceutically acceptable salt thereof, or a pharmaceutical composition thereof as defined herein.

A “MALT1 protease inhibitor” as defined herein includes any compound exhibiting MALT1 inhibition activity. In some embodiments, a MALT1 protease inhibitor is selective for a MALT1 protease. Exemplary MALT1 protease inhibitors can exhibit inhibition activity (IC₅₀) against a MALT1 protease of less than about 1000 nM, less than about 500 nM, less than about 200 nM, less than about 100 nM, less than about 50 nM, less than about 25 nM, less than about 10 nM, or less than about 1 nM as measured in an assay as described herein. In some embodiments, a MALT1 protease inhibitor can exhibit inhibition activity (IC₅₀) against a MALT1 protease of less than about 25 nM, less than about 10 nM, less than about 5 nM, or less than about 1 nM as measured in an assay as provided herein.

As used herein, a “first MALT1 protease inhibitor” or “first MALT1 inhibitor” is a MALT1 protease inhibitor as defined herein, but which does not include a compound of Formula (I), or a pharmaceutically acceptable salt thereof as defined herein. As used herein, a “second MALT1 protease inhibitor” or a “second MALT1 inhibitor” is a MALT1 protease inhibitor as defined herein, but which does not include a compound of Formula (I), or a pharmaceutically acceptable salt thereof as defined herein. When both a first and a second MALT1 inhibitor are present in a method provided herein, the first and second MALT1 protease inhibitor are different.

Exemplary first and second MALT1 protease inhibitors are described herein. In some embodiments, a first or second MALT1 protease inhibitor can be, for example, JNJ-67856633 or CTX-177.

The phrase “effective amount” means an amount of compound that, when administered to a subject in need of such treatment, is sufficient to (i) treat a MALT1-associated disease or disorder (such as a MALT1-associated cancer), (ii) attenuate, ameliorate, or eliminate one or more symptoms of the particular disease, condition, or disorder, or (iii) delay the onset of one or more symptoms of the particular disease, condition, or disorder described herein. The amount of a compound of Formula (I), or a pharmaceutically acceptable salt thereof that will correspond to such an amount will vary depending upon factors such as the particular compound, disease condition and its severity, the identity (e.g., weight) of the subject in need of treatment, but can nevertheless be routinely determined by one skilled in the art.

When employed as pharmaceuticals, compounds of Formula (I), including pharmaceutically acceptable salts thereof, can be administered in the form of pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, a compound of Formula (I) or pharmaceutically acceptable salt thereof, in combination with one or more pharmaceutically acceptable excipients. For example, a pharmaceutical composition prepared using a compound of Formula (I) or a pharmaceutically acceptable salt thereof. In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing a compound of Formula (I) or a pharmaceutically acceptable salt thereof with a pharmaceutically acceptable carrier. Pharmaceutical compositions containing a compound of Formula (I) or a pharmaceutically acceptable salt thereof as the active ingredient can be prepared by intimately mixing the compound of Formula (I), or a pharmaceutically acceptable salt thereof with a pharmaceutical carrier according to conventional pharmaceutical compounding techniques. The carrier can take a wide variety of forms depending upon the desired route of administration (e.g., oral, parenteral). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets. Second Edition. Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In preparing the compositions in oral dosage form, any of the usual pharmaceutical media can be employed. Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Solid oral preparations can also be coated with substances such as sugars or be enteric-coated so as to modulate major site of absorption. For parenteral administration, the carrier will usually consist of sterile water and other ingredients can be added to increase solubility or preservation. Injectable suspensions or solutions can also be prepared utilizing aqueous carriers along with appropriate additives. The pharmaceutical compositions herein will contain, per dosage unit, e.g., tablet, capsule, powder, injection, teaspoonful and the like, an amount of the active ingredient necessary to deliver an effective dose as described herein.

The compositions comprising a compound of Formula (I) or a pharmaceutically acceptable salt thereof can be formulated in a unit dosage form, each dosage containing from about 5 to about 1,000 mg (1 g), more usually about 100 mg to about 500 mg, of the active ingredient. The term “unit dosage form” refers to physically discrete units suitable as unitary dosages for human subjects and other subjects, each unit containing a predetermined quantity of active material (i.e., a compound of Formula (I) or a pharmaceutically acceptable salt thereof) calculated to produce the desired therapeutic effect, in association with a suitable pharmaceutical excipient.

In some embodiments, the compositions provided herein contain from about 5 mg to about 50 mg of the active ingredient. One having ordinary skill in the art will appreciate that this embodies compounds or compositions containing about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg of the active ingredient.

In some embodiments, the compositions provided herein contain from about 50 mg to about 500 mg of the active ingredient. One having ordinary skill in the art will appreciate that this embodies compounds or compositions containing about 50 mg to about 100 mg, about 100 mg to about 150 mg, about 150 mg to about 200 mg, about 200 mg to about 250 mg, about 250 mg to about 300 mg, about 350 mg to about 400 mg, or about 450 mg to about 500 mg of the active ingredient. In some embodiments, the compositions provided herein contain about 10 mg, about 20 mg, about 80 mg, or about 160 mg of the active ingredient.

In some embodiments, the compositions provided herein contain from about 500 mg to about 1,000 mg of the active ingredient. One having ordinary skill in the art will appreciate that this embodies compounds or compositions containing about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, or about 950 mg to about 1,000 mg of the active ingredient.

The daily dosage of the compound of Formula (I) or a pharmaceutically acceptable salt thereof can be varied over a wide range from 1.0 to 10,000 mg per adult human per day, or higher, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 160, 200, 250 and 500 milligrams of the active ingredient for the symptomatic adjustment of the dosage to the subject to be treated. An effective amount of the drug is ordinarily supplied at a dosage level of from about 0.1 mg/kg to about 1000 mg/kg of body weight per day, or any range therein. Preferably, the range is from about 0.5 to about 500 mg/kg of body weight per day, or any range therein. More preferably, from about 1.0 to about 250 mg/kg of body weight per day, or any range therein. More preferably, from about 0.1 to about 100 mg/kg of body weight per day, or any range therein. In an example, the range can be from about 0.1 to about 50.0 mg/kg of body weight per day, or any amount or range therein. In another example, the range can be from about 0.1 to about 15.0 mg/kg of body weight per day, or any range therein. In yet another example, the range can be from about 0.5 to about 7.5 mg/kg of body weight per day, or any amount to range therein. Pharmaceutical compositions containing a compound of Formula (I) or a pharmaceutically acceptable salt thereof can be administered on a regimen of 1 to 4 times per day or in a single daily dose.

The active compound may be effective over a wide dosage range and is generally administered in a pharmaceutically effective amount. Optimal dosages to be administered can be readily determined by those skilled in the art. It will be understood, therefore, that the amount of the compound actually administered will usually be determined by a physician, and will vary according to the relevant circumstances, including the mode of administration, the actual compound administered, the strength of the preparation, the condition to be treated, and the advancement of the disease condition. In addition, factors associated with the particular subject being treated, including subject response, age, weight, diet, time of administration and severity of the subject's symptoms, will result in the need to adjust dosages.

In some embodiments, the compounds provided herein can be administered in an amount ranging from about 1 mg/kg to about 100 mg/kg. In some embodiments, the compound provided herein can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. For example, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

One skilled in the art will recognize that both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy subjects and/or those suffering from a given disorder, can be completed according to methods well known in the clinical and medical arts.

Provided herein are pharmaceutical kits useful, for example, in the treatment of MALT1-associated diseases or disorders, such as cancer, which include one or more containers containing a pharmaceutical composition comprising an effective amount of a compound provided herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

### Materials and Methods

The compounds provided herein, including salts thereof, can be prepared using known organic synthesis techniques and can be synthesized according to any of numerous possible synthetic routes.

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in Protecting Group Chemistry, 1^{st} Ed., Oxford University Press, 2000; March's Advanced Organic Chemistry: Reactions, Mechanisms, and Structure, 5^{th} Ed., Wiley-Interscience Publication, 2001; and Peturssion, S. et al., “Protecting Groups in Carbohydrate Chemistry,” J. Chem. Educ., 74(11), 1297 (1997).

Reactions sensitive to moisture or air were performed under nitrogen or argon using anhydrous solvents and reagents. The progress of reactions was determined by either analytical thin layer chromatography (TLC) usually performed with Sanpont precoated TLC plates, silica gel GF-254, layer thickness 0.25 mm or liquid chromatography-mass spectrometry (LC-MS). Typically, the analytical LC-MS system used consisted of Shimadzu LCMS-2020 with electrospray ionization in positive ion detection mode with 20ADXR pump, SIL-20ACXR autosampler, CTO-20AC column oven, M20A PDA Detector and LCMS 2020 MS detector. The column was usually HALO a C18 30*5.0 mm, 2.7 μm. The mobile phase A is water containing 0.05% TFA and mobile phase B is acetonitrile containing 0.05% TFA. The gradient is from 5% mobile phase B to 100% in 2.0 min, hold 0.7 min, then reverting to 5% mobile phase B over 0.05 min and maintained for 0.25 min. The Column Oven (CTO-20AC) was operated at a temperature of 40.0° C. The flow rate was 1.5 mL/min, and the injection volume was 1 μl. PDA (SPD-M20A) detection was in the range 190-400 nm. The MS detector, which was configured with electrospray ionization as ionizable source; Acquisition mode: Scan; Nebulizing Gas Flow: 1.5 L/min; Drying Gas Flow: 15 L/min; Detector Voltage: Tuning Voltage±0.2 kv; DL Temperature: 250° C.; Heat Block Temperature: 250° C.; Scan Range: 90.00-900.00 m/z. ELSD (Alltech 3300) detector Parameters: Drift Tube Temperature: 60±5° C.; N2 Flow-Rate: 1.8±0.2 L/min. Mobile phase gradients were optimized for the individual compounds.

The GC-MS system was usually performed with Shimadzu GCMS-QP2010 Ultra with FID and MS Detector. The MS detector of acquisition mode: Start Time: 2.00 min; End Time: 9.00 min; ACQ Mode: Scan; Event Time: 0.30 sec; Scan Speed: 2000; Start m/z: 50.00; End m/z: 550.00; Ion Source temperature: 200.00° C.; Interface temperature: 250.00° C.; Solvent Cut Time: 2.00 min.

Preparative HPLC purifications were usually performed with Waters Auto purification system (2545-2767) with a 2489 UV detector. The column was Waters C18, 19×150 mm, 5 μm. The mobile phases consisted of mixtures of acetonitrile (5-95%) in water containing 0.1% FA. Flow rates were maintained at 25 mL/min, the injection volume was 1200 μL, and the UV detector used two channels 254 nm and 220 nm. Mobile phase gradients were optimized for the individual compounds.

Chiral analytical chromatography was performed on one of Chiralpak AS, AD, Chiralcel OD, OJ Chiralpak IA, IB, IC, ID, IE, IF, IG, IH columns (Daicel Chemical Industries, Ltd.); (R,R)-Whelk-O1, (S,S)-Whelk-O1 columns (Regis technologies, Inc.); CHIRAL Cellulose-SB, SC, SA columns (YMC Co., Ltd.) at different column sizes (50×4.6 mm, 100×4.6 mm, 150×4.6 mm, 250×4.6 mm, 50×3.0 mm, 100×3.0 mm) with noted percentage of either ethanol in hexane (% Et/Hex) or isopropanol in hexane (% IPA/Hex) as isocratic solvent systems.

Reactions performed using microwave irradiation were normally carried out using an Initiator manufactured by Biotage. Concentration of solutions was carried out on a rotary evaporator under reduced pressure. Flash column chromatography was usually performed using a Biotage Flash Chromatography apparatus (Dyax Corp.) on silica gel (40-60 μM, 60 Å pore size) in pre-packed cartridges of the size noted. ¹H NMR spectra were acquired at 400 MHz spectrometers in DMSO-d₆ solutions unless otherwise noted. Chemical shifts were reported in parts per million (ppm). Tetramethylsilane (TMS) was used as internal reference in DMSO-d₆ solutions, and residual CH₃OH peak or TMS was used as internal reference in CD₃OD solutions. Coupling constants (J) were reported in hertz (Hz). Chiral analytical chromatography was performed on one of Chiralpak AS, Chiralpak AD, Chiralcel OD, Chiralcel IA, or Chiralcel OJ columns (250×4.6 mm) (Daicel Chemical Industries, Ltd.) with noted percentage of either ethanol in hexane (% Et/Hex) or isopropanol in heptane (% IPA/Hep) as isocratic solvent systems. Chiral preparative chromatography was conducted on one of Chiralpak AS, AD, Chiralcel OD, OJ, Chiralpak IA, IB, IC, ID, IE, IF, IG, IH columns (Daicel Chemical Industries, Ltd.); (R,R)-Whelk-O1, (S,S)-Whelk-O1 columns (Regis technologies, Inc.); CHIRAL Cellulose-SB, SC, SA columns (YMC Co., Ltd.) at different column size (250×20 mm, 250×30 mm, 250×50 mm) with desired isocratic solvent systems identified on chiral analytical chromatography.

Abbreviations used herein include: —C(O)CH₃ (Ac); acetic acid (AcOH); —OC(O)CH₃ (OAc); aqueous (aq); Cbz (benzyloxycarbonyl); N,N-diisopropylethylamine (DIEA); N;N-dimethylformamide (DMF); 1-Ethyl-3-(3-dimethylaminopropyl)carbodiimide (EDCI); ethyl acetate (EtOAc); diethyl ether (ether or Et₂O); petroleum ether (PE); gram(s) (g); hour(s) (h or hr); 2-propanol (IPA); mass spectrum (ms or MS); microliter(s) (μL); milligram(s) (mg); milliliter(s) (mL); millimole (mmol); minute(s) (min); methyl t-butylether (MTBE); (benzotriazol-1-yloxy)tripyrrolidino-phosphonium hexafluorophosphate (PyBOP); retention time (Rt); rt (rt or RT); saturated aq sodium chloride solution (brine); trifluoroacetic acid (TFA); tetrahydrofuran (THF); flash chromatography (FC); liquid chromatography (LC); liquid chromatography-mass spectrometry (LCMS or LC-MS); supercritical fluid chromatography (SFC); t-butyloxycarbonyl (Boc or BOC); Diethylaminosulfur trifluoride (DAST); dichloromethane (DCM); dimethylacetamide (DMA or DMAC); dimethylsulfoxide (DMSO); toluene (tol); 1,3-Bis(diphenylphosphino)propane (DPPP); acetic acid (HOAc); 3-chloroperoxybenzoic acid (m-CPBA); methyl (Me); methanol (MeOH); chloro-oxido-dioxo-chromium; pyridin-1-ium (PCC); N-bromosuccinamide (NBS); thin layer chromatography (TLC).

The following are representative procedures for the preparation of the compounds used in the following Examples, or which can be substituted for the compounds used in the following Examples which may not be commercially available.

### Method A1

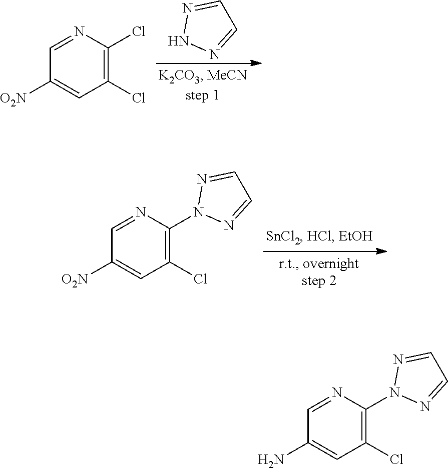 [SMILES: O=[N+]([O-])c1cnc(Cl)c(Cl)c1 ; c1cn[nH]n1 ; O=[N+]([O-])c1cnc(-n2nccn2)c(Cl)c1 ; Nc1cnc(-n2nccn2)c(Cl)c1]

### Step 1: 3-chloro-5-nitro-2-(2H-1,2,3-triazol-2-yl)pyridine

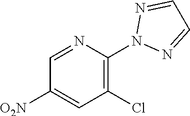 [SMILES: O=[N+]([O-])c1cnc(-n2nccn2)c(Cl)c1]

Into a 500 mL flask were placed 2,3-dichloro-5-nitropyridine (22.8 g, 118.2 mmol, 1.0 equiv.), CH₃CN (250 mL), 2H-1,2,3-triazole (9.0 g, 130.0 mmol, 1.1 equiv.), and K₂CO₃ (21.2 g, 153.6 mmol, 1.3 equiv.). The resulting mixture was stirred for 15 h at 40° C. The mixture was allowed to cool down to 25° C. The mixture was poured into 300 mL of EtOAc. The organic layers were washed with H₂O (2×300 mL) and brine (300 mL), dried over anhydrous Na₂SO₄, filtered and concentrated under reduced pressure. To the residue was added CH₂Cl₂ (50 mL). The resulting mixture was filtered. The filter cake was washed with CH₂Cl₂ (2×10 mL) and dried to give 3-chloro-5-nitro-2-(1,2,3-triazol-2-yl)pyridine (6.8 g, 26% yield) as an off-white solid. ¹H NMR (400 MHz, DMSO-d₆) δ: 8.39 (d, J=2.4 Hz, 1H), 8.14 (d, J=2.4 Hz, 1H), 8.33 (s, 2H). LC-MS: m/z 226 [M+H]⁺.

### Step 2: 5-chloro-6-(2H-1,2,3-triazol-2-yl)pyridin-3-amine

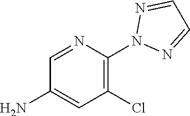 [SMILES: Nc1cnc(-n2nccn2)c(Cl)c1]

Into a 1.0 L flask were placed 3-chloro-5-nitro-2-(1,2,3-triazol-2-yl)pyridine (6.6 g, 29.3 mmol, 1.0 equiv.) and EtOH (200 mL). HCl (50 mL) was added at 0° C. Then SnCl₂ dihydrate (33.0 g, 146.3 mmol, 5.0 equiv.) was added at 0° C. by portions. The resulting mixture was stirred for 15 h at 25° C. The mixture was concentrated under reduced pressure and the residue was dissolved in water (300 mL). The mixture was basified to pH 9 with 3N NaOH. The resulting mixture was extracted with EtOAc (2×400 mL). The combined organic layers were washed with brine (500 mL), dried over anhydrous Na₂SO₄, filtered and concentrated under reduced pressure to give 5-chloro-6-(1,2,3-triazol-2-yl)pyridin-3-amine (5.4 g, 94% yield) as an off-white solid. ¹H NMR (300 MHz, DMSO-d₆) δ: 8.05 (s, 2H), 7.83 (d, J=2.5 Hz, 1H), 7.21 (d, J=2.5 Hz, 1H), 6.19 (s, 2H). LC-MS: m/z 196 [M+H]⁺.

### Method B1

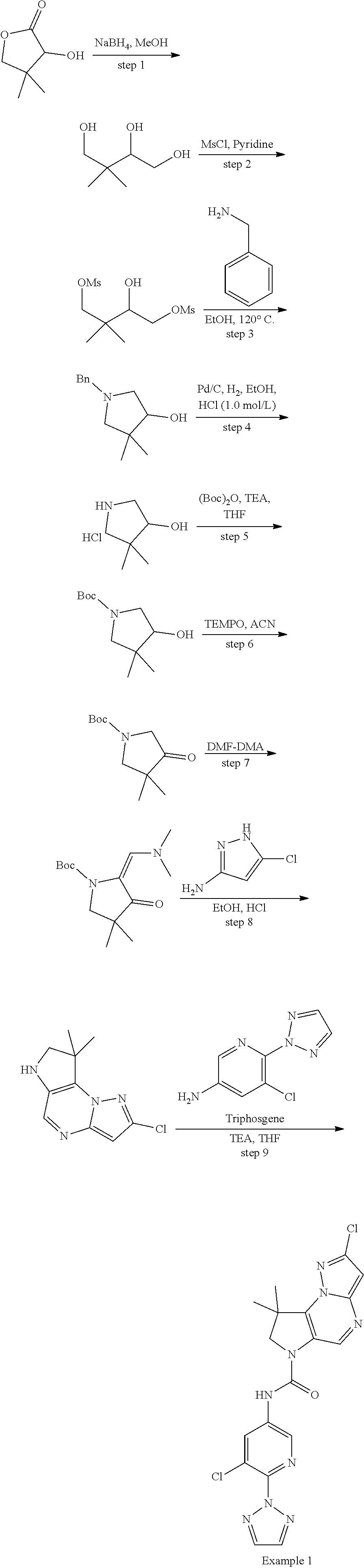

### Example 1: 2-chloro-N-(5-chloro-6-(2H-1,2,3-triazol-2-yl)pyridin-3-yl)-8,8-dimethyl-7,8-dihydro-6H-pyrazolo[1,5-a]pyrrolo[2,3-e]pyrimidine-carboxamide

### Step 1: 3,3-dimethylbutane-1,2,4-triol

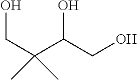

Into a 2.0 L 3-necked flask were placed pantolactone (26.0 g, 199.8 mnmol, 1.0 equiv.) and MeOH (800 mL). NaBH₄ (18.9 g, 499.5 mnmol, 2.5 equiv.) was added at 0° C. by portions. The resulting mixture was stirred for 4 h at 25° C. The mixture was acidified to pH 7 with 1N HCl. The resulting mixture was concentrated under reduced pressure. MeOH (200 mL) was added and the solid was filtered out. The filtrate was concentrated under vacuum. The residue was applied on a silica gel column and eluted with CH₂Cl₂/MeOH (10:1) to afford 3,3-dimethylbutane-1,2,4-triol (21.0 g, 78% yield) as a yellow oil. ¹H NMR (400 MHz, CDCl₃) δ: 4.36 (s, 3H), 3.70-3.30 (m, 5H), 0.87-0.83 (m, 6H).

### Step 2: 3-hydroxy-2,2-dimethylbutane-1,4-diyl dimethanesulfonate

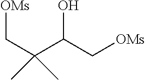 [SMILES: CC(C)(COS(C)(=O)=O)C(O)COS(C)(=O)=O]

Into a 500 mL 3-necked flask were placed 3,3-dimethylbutane-1,2,4-triol (21.0 g, 156.5 mmol, 1.0 equiv.) and pyridine (150 mL). Methanesulfonyl chloride (35.9 g, 312.9 mmol, 2.0 equiv.) was added dropwise at 0° C. The resulting mixture was stirred for 18 h at 25° C. The mixture was poured into DCM (200 mL). The mixture was acidified to pH 2 with 2N HCl. The resulting mixture was extracted with CH₂Cl₂ (3×300 mL). The combined organic layers were dried over anhydrous Na₂SO₄. After filtration, the filtrate was concentrated under reduced pressure. The residue was applied on a silica gel column and eluted with CH₂Cl₂/MeOH (25:1) to afford 2-hydroxy-4-(methanesulfonyloxy)-3,3-dimethylbutyl methanesulfonate (17.4 g, 38% yield) as a red oil. ¹H NMR (400 MHz, CDCl₃) δ: 4.35-4.31 (m, 1H), 4.19-4.11 (m, 2H), 3.88 (d, J=9.4 Hz, 1H), 3.81-3.78 (m, 1H), 3.05 (s, 3H), 3.02 (s, 3H), 1.02 (s, 3H), 0.94 (s, 3H). LC-MS: m/z 291 (M+H)⁺.

### Step 3: 1-benzyl-4,4-dimethylpyrrolidin-3-ol

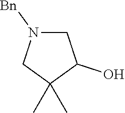

Into a 150 mL pressure tank reactor were placed 2-hydroxy-4-(methanesulfonyloxy)-3,3-dimethylbutyl methanesulfonate (15.0 g, 51.6 mmol, 1.0 equiv.), EtOH (70 mL) and benzylamine (16.6 g, 154.9 mmol, 3.0 equiv.). The resulting mixture was stirred for 18 h at 120° C. The mixture was allowed to cool down to 25° C. and concentrated under vacuum. Et₂O (500 mL) was added and the solid was filtered out. The filtrate was concentrated under vacuum. The residue was applied on a silica gel column and eluted with CH₂Cl₂/MeOH (30:1) to afford 1-benzyl-4,4-dimethylpyrrolidin-3-ol (5.5 g, 52% yield) as a red oil. ¹H NMR (400 MHz, CDCl₃) δ: 7.32-7.22 (m, 5H), 3.75-3.73 (m, 1H), 3.62 (s, 2H), 2.95-2.91 (m, 1H), 2.59-2.52 (m, 2H), 2.31-2.24 (m, 2H), 1.06 (s, 6H). LC-MS: m/z 206 (M+H)⁺.

### Step 4: 4,4-dimethylpyrrolidin-3-ol hydrochloride

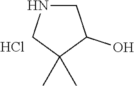

Into a 500 mL flask were placed 1-benzyl-4,4-dimethylpyrrolidin-3-ol (5.6 g, 27.4 mmol, 1.0 equiv.), EtOH (140 mL), 1N HCl (30 mL) and Pd/C (500 mg). The resulting mixture was stirred for 18 h at 25° C. under hydrogen atmosphere. The resulting mixture was filtered. The filtrate was concentrated under reduced pressure to afford 4,4-dimethylpyrrolidin-3-ol hydrochloride (4.0 g, 96% yield) as a light yellow solid. ¹H NMR (400 MHz, DMSO-d₆) δ: 3.79-3.77 (m, 1H), 3.47-3.35 (m, 1H), 3.00-2.86 (m, 3H), 1.00 (s, 3H), 0.97 (s, 3H). LC-MS: m/z 116 (M+H)⁺.

### Step 5: tert-butyl 4-hydroxy-3,3-dimethylpyrrolidine-1-carboxylate

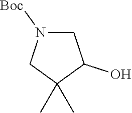

Into a 500 mL flask were placed 4,4-dimethylpyrrolidin-3-ol hydrochloride (4.0 g, 26.3 mmol, 1.0 equiv.), THF (100 mL), (Boc)₂O (8.6 g, 39.5 mmol, 1.5 equiv.), and TEA (13.4 g, 131.9 mmol, 5.0 equiv.). The resulting mixture was stirred for 2 h at 25° C. The resulting mixture was concentrated under vacuum. The residue was applied on a silica gel column and eluted with CH₂Cl₂/MeOH (20:1) to afford tert-butyl 4-hydroxy-3,3-dimethylpyrrolidine-1-carboxylate (5.5 g, 96% yield) as a yellow oil. ¹H NMR (400 MHz, CDCl₃) δ: 3.94-3.79 (m, 1H), 3.74-3.64 (m, 1H), 3.34-3.06 (m, 3H), 1.46 (s, 9H), 1.07 (s, 3H), 1.02 (s, 3H). LC-MS: m/z 216 (M+H)⁺.

### Step 6: tert-butyl 3,3-dimethyl-4-oxopyrrolidine-1-carboxylate

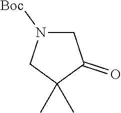

Into a 250 mL flask were placed tert-butyl 4-hydroxy-3,3-dimethylpyrrolidine-1-carboxylate (5.0 g, 23.2 mmol, 1.0 equiv.), ACN (60 mL), N-methylmorpholine N-oxide (3.5 g, 30.2 mmol, 1.3 equiv.) and TPAP (408 mg, 1.2 mmol, 0.05 equiv.). The resulting mixture was stirred for 1.5 h at 25° C. The resulting mixture was concentrated under vacuum. The residue was applied on a silica gel column and eluted with PE/EtOAc (8:1) to afford tert-butyl 3,3-dimethyl-4-oxopyrrolidine-1-carboxylate (3.4 g, 68% yield) as a colorless oil. ¹H NMR (300 MHz, DMSO-d₆) δ: 3.80 (s, 2H), 3.45 (s, 2H), 1.43 (s, 9H), 1.06 (s, 6H). LC-MS: m/z 214 (M+H)⁺.

### Step 7: tert-butyl (E)-2-((dimethylamino)methylene)-4,4-dimethyl-3-oxopyrrolidine-1-carboxylate

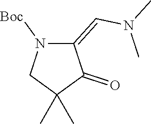 [SMILES: CN(C)/C=C1\C(=O)C(C)(C)CN1C(=O)OC(C)(C)C]

Into a 250 mL flask were placed tert-butyl 3,3-dimethyl-4-oxo-pyrrolidine-1-carboxylate (3.4 g, 15.9 mmol) and DMF-DMA (35 mL). The mixture was stirred for 5 h at 105° C. The reaction mixture was cooled to 20° C. and concentrated under vacuum to afford tert-butyl (E)-2-((dimethylamino)methylene)-4,4-dimethyl-3-oxopyrrolidine-1-carboxylate (4.3 g, crude) as a yellow oil. LC-MS: m/z 269 (M+H)⁺.

### Step 8: 2-chloro-8,8-dimethyl-7,8-dihydro-6H-pyrazolo[1,5-a]pyrrolo[2,3-e]pyrimidine

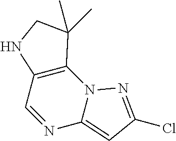

A mixture of tert-butyl (2E)-2-(dimethylaminomethylene)-4,4-dimethyl-3-oxo-pyrrolidine-1-carboxylate (4.2 g, 15.6 mmol), 5-chloro-1H-pyrazol-3-amine (1.8 g, 15.6 mmol), EtOH (45 mL) and HCl (4N, 22.5 mL) was split and equally placed into three 40 mL vials. The vials were stirred for 1.5 h at 80° C. The reaction mixtures were cooled to 25° C., combined and concentrated under vacuum. To the residue was added aq. NaHCO₃(50 mL) and the resulting mixture was extracted with EtOAc (3×50 mL). The organic layers were combined, dried over Na₂SO₄ and concentrated under vacuum. The residue was applied on a silica gel column and eluted with MeOH/DCM (1/20) to give 2-chloro-8,8-dimethyl-7,8-dihydro-6H-pyrazolo[1,5-a]pyrrolo[2,3-e]pyrimidine (300 mg, 9% yield) as a yellow oil. ¹H NMR (400 MHz, CDCl₃) δ: 8.22 (s, 1H), 6.60 (s, 1H), 3.52 (s, 2H), 1.66 (s, 6H). LC-MS: m/z 223 (M+H)⁺.

### Step 9: 2-chloro-N-(5-chloro-6-(2H-1,2,3-triazol-2-yl)pyridin-3-yl)-8,8-dimethyl-7,8-dihydro-6H-pyrazolo[1,5-a]pyrrolo[2,3-e]pyrimidine-6-carboxamide

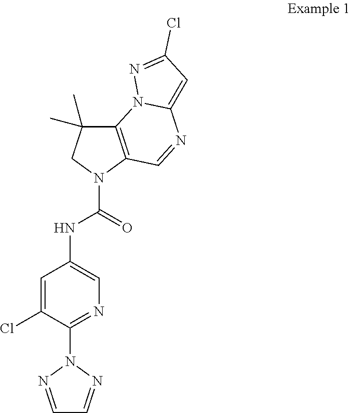 [SMILES: CC1(C)CN(C(=O)Nc2cnc(-n3nccn3)c(Cl)c2)c2cnc3cc(Cl)nn3c21]

Into a 40 mL vial were placed 5-chloro-6-(triazol-2-yl)pyridin-3-amine (Method A1 step 2; 179 mg, 0.9 mmol), THF (10 mL), triphosgene (113 mg, 0.4 mmol), and N,N-diethylethanamine (100 mg, 1.0 mmol). The mixture was stirred for 0.5 h at 25° C. The formed solid was filtered off and 2-chloro-8,8-dimethyl-7,8-dihydro-6H-pyrazolo[1,5-a]pyrrolo[2,3-e]pyrimidine (170 mg, 0.8 mmol) and N,N-diethylethanamine (313 mg, 3.1 mmol) were added into the filtrate. The mixture was stirred for 15 h at 25° C. The reaction mixture was concentrated under vacuum. The residue was applied on a silica gel column and eluted with MeOH/DCM (1/30) to give 160 mg crude product as a yellow solid. The crude product was purified by Prep-HPLC. The collected fractions were lyophilized to give 2-chloro-N-(5-chloro-6-(2H-1,2,3-triazol-2-yl)pyridin-3-yl)-8,8-dimethyl-7,8-dihydro-6H-pyrazolo[1,5-a]pyrrolo[2,3-e]pyrimidine-6-carboxamide (22.2 mg, 6% yield) as a white solid. H NMR (400 MHz, DMSO-d₆) δ: 9.50 (s, 1H), 9.28 (s, 1H), 8.78 (d, J=2.0 Hz, 1H), 8.54 (d, J=2.4 Hz, 1H), 8.18 (s, 2H), 6.94 (s, 1H), 4.19 (s, 2H), 1.69 (s, 6H). LC-MS: m/z 444 (M+H)⁺.

### Method C1

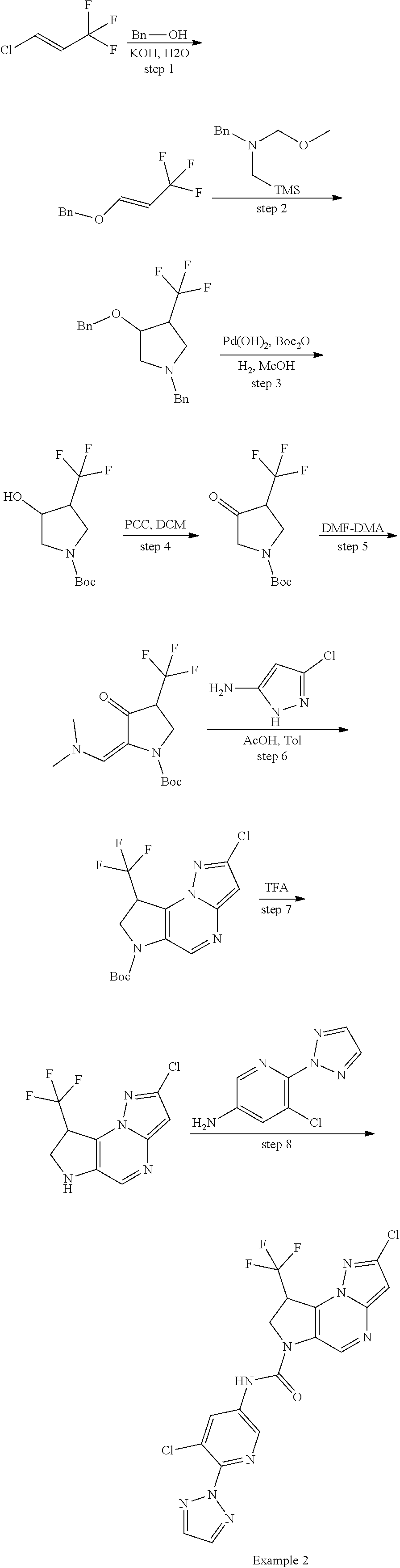

### Example 2: 2-chloro-N-(5-chloro-6-(2H-1,2,3-triazol-2-yl)pyridin-3-yl)-8-(trifluoromethyl)-7,8-dihydro-6H-pyrazolo[1,5-a]pyrrolo[2,3-]pyrimidine-6-carboxamide

### Step 1: (E)-(((3,3,3-trifluoroprop-1-en-1-yl)oxy)methyl)benzene

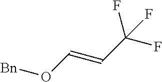 [SMILES: FC(F)(F)/C=C/OCc1ccccc1]

Into a 150 mL pressure tank reactor were added phenylmethanol (37.3 g, 344.8 mmol), H₂O (6.2 g, 344.8 mmol), and potassium hydroxide (19.4 g, 344.8 mmol). (E)-1-chloro-3,3,3-trifluoro-prop-1-ene (22.5 g, 172.4 mmol) was added at −20° C. The mixture was stirred for 1 h at 22° C. and then for another 12.0 h at 70° C. The mixture was cooled to 25° C. The solid was filtered out. The filtrate was concentrated under vacuum. The residue was applied on a silica gel column and eluted with EtOAc/hexane (1/25) to give (E)-(((3,3,3-trifluoroprop-1-en-1-yl)oxy)methyl)benzene (11.5 g, 29% yield) as a colorless liquid. ¹H NMR (300 MHz, CDCl₃) δ: 7.49-7.35 (m, 5H), 7.22-7.13 (m, 1H), 5.19-5.04 (m, 1H), 4.86 (s, 2H).

### Step 2: 1-benzyl-3-(benzyloxy)-4-(trifluoromethyl)pyrrolidine

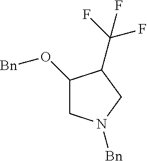 [SMILES: FC(F)(F)C1CN(Cc2ccccc2)CC1OCc1ccccc1]

Into a 250 mL 3-necked flask were placed (E)-(((3,3,3-trifluoroprop-1-en-1-yl)oxy)methyl)benzene (8.9 g, 44.1 mmol) and N-(methoxymethyl)-1-phenyl-N-(trimethylsilylmethyl)methanamine (15.7 g, 66.2 mmol), followed by the dropwise addition of 2,2,2-trifluoroacetic acid (503 mg, 4.4 mmol) at 0° C. The mixture was stirred for 5 h at 25° C. and poured into 150 mL of NaHCO₃ (aq). The resulting solution was extracted with 3×150 mL of EtOAc. The organic layers were combined, dried and concentrated under vacuum. The residue was applied on a silica gel column and eluted with EtOAc/hexane (1/20) to give 1-benzyl-3-benzyloxy-4-(trifluoromethyl)pyrrolidine (5.0 g, 30% yield) as a colorless liquid. LC-MS: m/z 336 (M+H)⁺.

### Step 3: tert-butyl 3-hydroxy-4-(trifluoromethyl)pyrrolidine-1-carboxylate

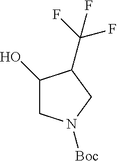 [SMILES: CC(C)(C)OC(=O)N1CC(O)C(C(F)(F)F)C1]

Into a 250 mL flask were placed 1-benzyl-3-benzyloxy-4-(trifluoromethyl)pyrrolidine (5.0 g, 14.9 mmol), MeOH (60 mL), (Boc)₂O (3.6 g, 16.4 mmol) and Pd(OH)₂/C (3.0 g). The flask was evacuated and flushed with nitrogen three times, followed by flushing with hydrogen. The mixture was stirred for 15 h at 25° C. under an atmosphere of hydrogen (balloon). The solid was filtered out. The filtrate was concentrated under vacuum. The residue was applied on a silica gel column and eluted with EtOAc/hexane (1/3) to give tert-butyl 3-hydroxy-4-(trifluoromethyl)pyrrolidine-1-carboxylate (3.7 g, 77% yield) as a colorless oil. ¹H NMR (300 MHz, CDCl₃) δ: 4.60-4.53 (m, 1H), 3.90-3.65 (m, 2H), 3.57-3.30 (m, 2H), 2.97-2.90 (m, 1H), 2.70-2.45 (m, 1H), 1.48 (s, 9H). LC-MS: m/z 256 (M+H)⁺.

### Step 4: tert-butyl 3-oxo-4-(trifluoromethyl)pyrrolidine-1-carboxylate

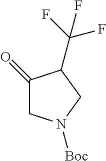 [SMILES: CC(C)(C)OC(=O)N1CC(=O)C(C(F)(F)F)C1]

Into a 250 mL flask were placed tert-butyl 3-hydroxy-4-(trifluoromethyl)pyrrolidine-1-carboxylate (2.2 g, 8.4 mmol), DCM (50 mL), pyridinium chlorochromate (PCC) (7.26 g, 33.7 mmol) and silica gel (2.0 g). The mixture was stirred for 48 h at 40° C. The solid was filtered out. The filtrate was concentrated under vacuum. The residue was applied on a silica gel column and eluted with EtOAc/hexane (1/10) to give tert-butyl 3-oxo-4-(trifluoromethyl)pyrrolidine-1-carboxylate (560 mg, 24% yield) as a colorless oil. ¹H NMR (300 MHz, CDCl₃) δ: 4.20-4.09 (m, 1H), 3.97-3.75 (m, 3H), 3.45-3.30 (m, 1H), 1.51 (s, 9H). LC-MS: m/z 254 (M+H)⁺.

### Step 5: tert-butyl (E)-2-((dimethylamino)methylene)-3-oxo-4-(trifluoromethyl)pyrrolidine-1-carboxylate

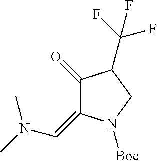 [SMILES: CN(C)/C=C1\C(=O)C(C(F)(F)F)CN1C(=O)OC(C)(C)C]

Into a 100 mL flask were placed tert-butyl 3-oxo-4-(trifluoromethyl)pyrrolidine-1-carboxylate (560 mg, 2.2 mmol) and DMF-DMA (6 mL). The mixture was stirred for 1 h at 35° C. The mixture was concentrated under vacuum to afford tert-butyl (2E)-2-(dimethylaminomethylene)-3-oxo-4-(trifluoromethyl)pyrrolidine-1-carboxylate (682 mg, crude) as a yellow oil. LC-MS: m/z 309 (M+H)⁺.

### Step 6: tert-butyl 2-chloro-8-(trifluoromethyl)-7,8-dihydro-6H-pyrazolo[1,5-a]pyrrolo[2,3-e]pyrimidine-6-carboxylate

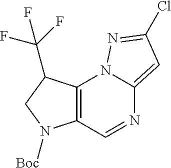 [SMILES: CC(C)(C)OC(=O)N1CC(C(F)(F)F)c2c1cnc1cc(Cl)nn21]

Into a 100 mL flask were placed tert-butyl 3-oxo-4-(trifluoromethyl)pyrrolidine-1-carboxylate (682 mg, 2.7 mmol), 3-chloro-1H-pyrazol-5-amine (316 mg, 2.7 mmol), toluene (10 mL) and AcOH (1 mL). The mixture was stirred for 15 h at 95° C. The reaction mixture was cooled to 25° C. and concentrated under vacuum. Then 20 mL of NaHCO₃(aq) was added. The resulting solution was extracted with 3×20 mL of EtOAc. The organic layers were combined, dried and concentrated under vacuum. The residue was applied on a silica gel column and eluted with EtOAc/hexane (1/20) to give tert-butyl 2-chloro-8-(trifluoromethyl)-7,8-dihydro-6H-pyrazolo[1,5-a]pyrrolo[2,3-e]pyrimidine-6-carboxylate (200 mg, 18% yield) as a yellow oil. ¹H NMR (300 MHz, DMSO-d₆) δ: 8.83-8.79 (m, 1H), 7.06 (s, 1H), 4.37-4.20 (m, 2H), 4.07-3.99 (m, 1H), 1.47 (s, 9H). LC-MS: m/z 363 (M+H)⁺.

### Step 7: 2-chloro-8-(trifluoromethyl)-7,8-dihydro-6H-pyrazolo[1,5-a]pyrrolo[2,3-e]pyrimidine

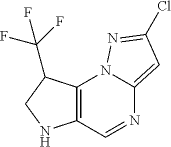 [SMILES: FC(F)(F)C1CNc2cnc3cc(Cl)nn3c21]

Into a 100 mL flask were placed tert-butyl 2-chloro-8-(trifluoromethyl)-7,8-dihydro-6H-pyrazolo[1,5-a]pyrrolo[2,3-e]pyrimidine-6-carboxylate (170 mg, 0.5 mmol), DCM (8 mL), and TFA (2 mL). The mixture was stirred for 1 h at 25° C. and concentrated under vacuum. Then 30 mL of NaHCO₃(aq) was added. The resulting solution was extracted with 3×40 mL of DCM. The organic layers were combined, dried and concentrated under vacuum. The residue was purified by thin layer chromatography developed with MeOH/DCM (1/35) to give 2-chloro-8-(trifluoromethyl)-7,8-dihydro-6H-pyrazolo[1,5-a]pyrrolo[2,3-e]pyrimidine (75 mg, 55% yield) as a yellow oil. LC-MS: m/z 263 (M+H)⁺.

### Step 8: 2-chloro-N-(5-chloro-6-(2H-1,2,3-triazol-2-yl)pyridin-3-yl)-8-(trifluoromethyl)-7,8-dihydro-6H-pyrazolo[1,5-a]pyrrolo[2,3-e]pyrimidine-6-carboxamide

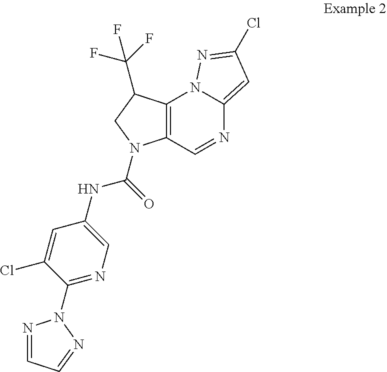 [SMILES: O=C(Nc1cnc(-n2nccn2)c(Cl)c1)N1CC(C(F)(F)F)c2c1cnc1cc(Cl)nn21]

Into a 40 mL vial were placed 5-chloro-6-(triazol-2-yl)pyridin-3-amine (Method A1 step 2; 67 mg, 0.3 mmol), THF (8 mL), bis(trichloromethyl)carbonate (51 mg, 0.2 mmol), and N,N-diethylethanamine (43 mg, 0.4 mmol). The mixture was stirred for 0.5 h at 25° C. The solid was filtered out. Then 2-chloro-8-(trifluoromethyl)-7,8-dihydro-6H-pyrazolo[1,5-a]pyrrolo[2,3-e]pyrimidine (75 mg, 0.29 mmol) and N,N-diethylethanamine (115 mg, 1.2 mmol) were added into the filtrate. The mixture was stirred for 15.0 h at 25° C. and concentrated under vacuum. The residue was applied to a silica gel column and eluted with MeOH/DCM (1/35) to give 101 mg of the crude material. The crude material was then subjected to purification using a Prep-HPLC. The collected fractions were lyophilized to give 2-chloro-N-(5-chloro-6-(2H-1,2,3-triazol-2-yl)pyridin-3-yl)-8-(trifluoromethyl)-7,8-dihydro-6H-pyrazolo[1,5-a]pyrrolo[2,3-e]pyrimidine-6-carboxamide (53.8 mg, 38% yield) as a light yellow solid. ¹H NMR (300 MHz, DMSO-d₆) δ: 9.72 (s, 1H), 9.33 (s, 1H), 8.77 (d, J=2.4 Hz, 1H), 8.52 (d, J=2.4 Hz, 1H), 8.18 (s, 2H), 7.08 (s, 1H), 5.49-5.26 (m, 1H), 4.77-4.72 (m, 1H), 4.62-4.58 (m, 1H). LC-MS: m/z 484 (M+H)⁺.

### Method D1

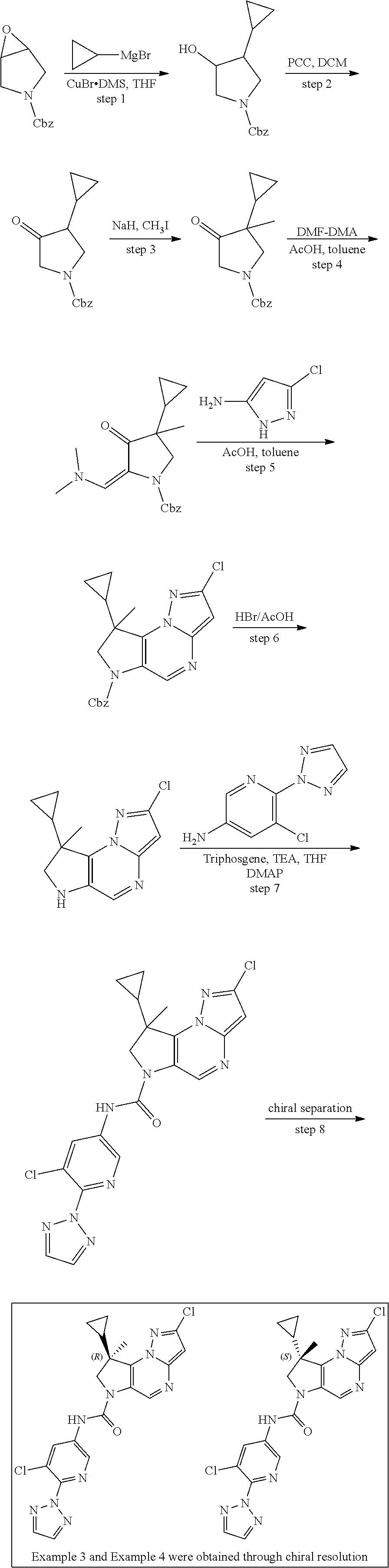

### Examples 3 and 4: Single enantiomers obtained from a racemic mixture containing (R)-2-chloro-N-(5-chloro-6-(2H-1,2,3-triazol-2-yl)pyridin-3-yl)-8-cyclopropyl-8-methyl-7,8-dihydro-6H-pyrazolo[1,5-a]pyrrolo[2,3-e]pyrimidine-6-carboxamide and (S)-2-chloro-N-(5-chloro-6-(2H-1,2,3-triazol-2-yl)pyridin-3-yl)-8-cyclopropyl-8-methyl-7,8-dihydro-6H-pyrazolo[1,5-a]pyrrolo[2,3-e]pyrimidine-6-carboxamide

### Step 1: Benzyl 3-cyclopropyl-4-hydroxypyrrolidine-1-carboxylate

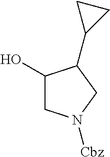 [SMILES: O=C(OCc1ccccc1)N1CC(O)C(C2CC2)C1]

To a stirred solution of benzyl 6-oxa-3-azabicyclo[3.1.0]hexane-3-carboxylate (1.2 g, 5.4 mmol) in THF (20 mL) was added cyclopropylmagnesium bromide (0.5 M, 32.4 mL) dropwise at −30° C. The resulting mixture was stirred for 1 h at −15° C. The solution was quenched with NH₄Cl (150 mL) and extracted with EtOAc (3×50 mL). The combined organic layers were concentrated under reduced pressure. The residue was purified by silica gel column chromatography (eluting with 0-100% EtOAc in hexane) to afford benzyl 3-cyclopropyl-4-hydroxy-pyrrolidine-1-carboxylate (1.0 g, 70% yield) as a light yellow oil. ¹H NMR (300 MHz, CDCl₃): 7.29-7.58 (m, 5H), 5.06 (s, 2H), 4.25-4.29 (m, 1H), 3.52-3.80 (m, 1H), 3.35-3.48 (m, 2H), 1.43-1.51 (m, 1H), 0.11-0.66 (m, 5H). LC-MS: m/z 262 [M+H]⁺.

### Step 2: Benzyl 3-cyclopropyl-4-oxo-pyrrolidine-1-carboxylate

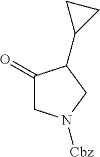 [SMILES: O=C1CN(C(=O)OCc2ccccc2)CC1C1CC1]

To a stirred mixture of benzyl 3-cyclopropyl-4-hydroxy-pyrrolidine-1-carboxylate (0.95 g, 3.64 mmol) in DCM (100 mL) was added pyridinium chlorochromate (PCC) (165.3 mg, 766.7 μmol). The resulting mixture was stirred for 16 h at 25° C. The solids were filtered out and washed with DCM (3×50 mL). The filtrate was concentrated under reduced pressure. The residue was purified by silica gel column chromatography (eluting with 0-100% EtOAc in hexane) to afford benzyl 3-cyclopropyl-4-oxo-pyrrolidine-1-carboxylate (0.6 g, 64% yield) as a light yellow oil. ¹H NMR (300 MHz, CDCl₃): 7.30-7.33 (m, 5H), 5.06 (s, 2H), 4.25-4.29 (m, 1H), 4.07-4.17 (m, 1H), 3.52-3.80 (m, 1H), 3.35-3.48 (m, 2H), 1.43-1.51 (m, 1H), 0.11-0.66 (m, 5H). LC-MS: m/z 260 [M+H]⁺.

### Step 3: Benzyl 3-cyclopropyl-3-meth 14-oxo-pyrrolidine-1-carboxylate

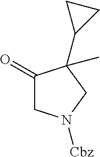 [SMILES: CC1(C2CC2)CN(C(=O)OCc2ccccc2)CC1=O]

To a stirred mixture of benzyl 3-cyclopropyl-4-oxo-pyrrolidine-1-carboxylate (1.00 g, 3.86 mmol) in THF (20 mL) was added sodium hydride (177.3 mg, 4.6 mmol, 60% in mineral oil) at 0° C. The resulting mixture was stirred for 1 h at 0° C. To the mixture was added CH₃I (547.6 mg, 3.9 mmol) dropwise. The resulting mixture was stirred for 0.5 h at 0° C. The mixture was quenched by pouring into sat. NH₄Cl (20 mL) and extracted with EtOAc (3×10 mL). The organic layers were washed with brine (2×20 mL), dried over anhydrous sodium sulfate, filtered and concentrated under reduced pressure. The residue was purified by silica gel column chromatography (eluting with 0-100% EtOAc in hexane) to afford benzyl 3-cyclopropyl-3-methyl-4-oxo-pyrrolidine-1-carboxylate (0.6 g, 48%) as a light yellow oil. ¹H NMR (300 MHz, CDCl₃): 7.39 (s, 5H), 5.20 (s, 2H), 3.95-4.29 (m, 1H), 3.35-3.58 (m, 1H), 1.28-1.35 (m, 4H), 0.22-0.55 (m, 4H). LC-MS: m/z 274 [M+H]⁺.

### Step 4: Benzyl (2E)-4-cyclopropyl-2-(dimethylaminomethylene)-4-methyl-3-oxo-pyrrolidine-1-carboxylate

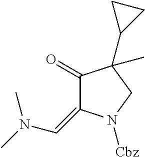

To benzyl 3-cyclopropyl-2,3-dimethyl-4-oxo-pyrrolidine-1-carboxylate (0.60 g, 2.09 mmol) was added 1,1-dimethoxy-N,N-dimethyl-methanamine (248.8 mg, 2.1 mmol, 279.6 μL). The mixture was stirred for 1 h at 80° C. The resulting mixture was cooled to rt and concentrated under reduced pressure to afford benzyl (2E)-4-cyclopropyl-2-(dimethylaminomethylene)-4-methyl-3-oxo-pyrrolidine-1-carboxylate (0.8 g, crude) as a red gum which was used for next step without further purification. LC-MS: m/z 329 [M+H]⁺.

### Step 5: Benzyl 2-chloro-8-cyclopropyl-8-methyl-7,8-dihydro-6H-pyrazolo[1,5-a]pyrrolo[2,3-e]pyrimidine-6-carboxylate

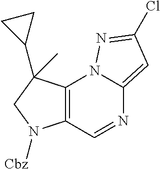 [SMILES: CC1(C2CC2)CN(C(=O)OCc2ccccc2)c2cnc3cc(Cl)nn3c21]

The mixture of 3-chloro-1H-pyrazol-5-amine (286.3 mg, 2.4 mmol) and benzyl (2E)-4-cyclopropyl-2-(dimethylaminomethylene)-4-methyl-3-oxo-pyrrolidine-1-carboxylate (0.8 g, 2.44 mmol) in toluene (10 mL) and AcOH (1 mL) was stirred for 4 h at 80° C. LCMS showed the reaction was complete. The mixture was concentrated in vacuo. The residue was purified by silica gel column chromatography (eluting with 0-100% EtOAc in hexane) to afford benzyl 2-chloro-8-cyclopropyl-8-methyl-7,8-dihydro-6H-pyrazolo[1,5-a]pyrrolo[2,3-e]pyrimidine-6-carboxylate (0.2 g, 24% yield) as a light yellow oil. ¹H NMR (300 MHz, CDCl₃): 9.22 (s, 1H), 7.32-7.53 (m, 5H), 6.67 (s, 1H), 5.18 (s, 2H), 3.58-3.73 (m, 1H), 1.07-1.35 (m, 4H), 0.22-0.64 (m, 4H). LC-MS: m/z 383 [M+H]⁺.

### Step 6: 2-chloro-8-cyclopropyl-8-methyl-7,8-dihydro-6H-pyrazolo[1,5-a]pyrrolo[2,3-e]pyrimidine

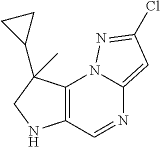 [SMILES: CC1(C2CC2)CNc2cnc3cc(Cl)nn3c21]

A solution of benzyl 2-chloro-8-cyclopropyl-8-methyl-7,8-dihydro-6H-pyrazolo[1,5-a]pyrrolo[2,3-e]pyrimidine-6-carboxylate (0.1 g, 261.2 μmol) in HBr/AcOH (1 mL, 13.6 μmol) was stirred for 3 h at 25° C. The mixture was concentrated in vacuo to give a crude product. The residue was diluted with EtOAc (30 mL), washed with saturated sodium bicarbonate (3×20 mL), brine (2×20 mL) and water (20 mL). The organic layer was dried over anhydrous sodium sulfate, filtered and concentrated under reduced pressure. The residue was purified by silica gel column chromatography (eluting with 0-100% EtOAc in hexane) to afford 2-chloro-8-cyclopropyl-8-methyl-7,8-dihydro-6H-pyrazolo[1,5-a]pyrrolo[2,3-e]pyrimidine (0.02 g, 31% yield) as a brown oil. ¹H NMR (300 MHz, CDCl₃): 8.37 (s, 1H), 6.65 (s, 1H), 3.38-3.41 (m, 1H), 1.68-1.55 (m, 4H), 0.44-0.84 (m, 4H). LC-MS: m/z 249 [M+H]⁺.

### Step 7: 2-chloro-N-(5-chloro-6-(2H-1,2,3-triazol-2-yl)pyridin-3-yl)-8-cyclopropyl-8-methyl-7,8-dihydro-6H-pyrazolo[1,5-a]pyrrolo[2,3-e]pyrimidine-6-carboxamide

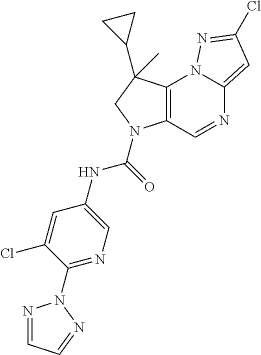 [SMILES: CC1(C2CC2)CN(C(=O)Nc2cnc(-n3nccn3)c(Cl)c2)c2cnc3cc(Cl)nn3c21]

To a stirred mixture of 5-chloro-6-(triazol-2-yl) pyridin-3-amine (Method A1 step 2; 40.0 mg, 204.5 μmol) and bis(trichloromethyl) carbonate (42.5 mg, 143.2 μmol) in THF (1 mL) was added TEA (62.1 mg, 613.5 μmol, 85.5 L) dropwise at 0° C. The resulting mixture was stirred for 1 h at 25° C. and filtered. The filtrate was added to a solution of 2-chloro-8-cyclopropyl-8-methyl-7,8-dihydro-6H-pyrazolo[1,5-a]pyrrolo[2,3-e]pyrimidine (25.4 mg, 102.2 μmol) in THF (1 mL). To this solution was added N,N-dimethylpyridin-4-amine (12.5 mg, 102.2 μmol) and it was stirred for 16 h at 25° C. The residue was diluted with EtOAc (300 mL), washed with brine (2×10 mL), dried over anhydrous sodium sulfate, filtered and concentrated under reduced pressure. The residue was first purified by silica gel column chromatography (eluting with 0-100% EtOAc in hexane), followed by prep-HPLC to afford 2-chloro-N-(5-chloro-6-(2H-1,2,3-triazol-2-yl)pyridin-3-yl)-8-cyclopropyl-8-methyl-7,8-dihydro-6H-pyrazolo[1,5-a]pyrrolo[2,3-e]pyrimidine-6-carboxamide (3.7 mg, 8% yield) as a racemic mixture. LC-MS: m/z 470 [M+H]⁺.

### Step 8: Separation of enantiomers to obtain (R)-2-chloro-N-(5-chloro-6-(2H-1,2,3-triazol-2-yl)pyridin-3-yl)-8-cyclopropyl-8-methyl-7,8-dihydro-6H-pyrazolo[1,5-a]pyrrolo[2,3-e]pyrimidine-6-carboxamide and (S)-2-chloro-N-(5-chloro-6-(2H-1,2,3-triazol-2-yl)pyridin-3-yl)-8-cyclopropyl-8-methyl-7,8-dihydro-6H-pyrazolo[1,5-a]pyrrolo[2,3-e]pyrimidine-6-carboxamide

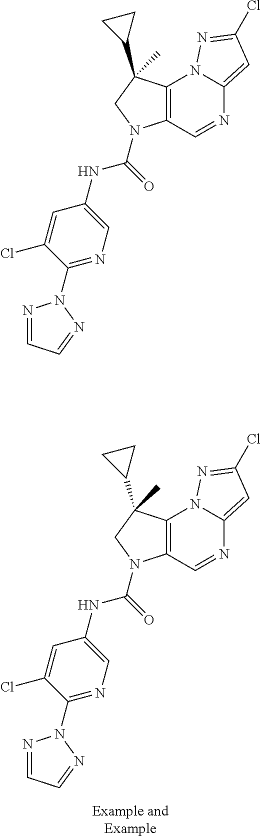 [SMILES: C[C@@]1(C2CC2)CN(C(=O)Nc2cnc(-n3nccn3)c(Cl)c2)c2cnc3cc(Cl)nn3c21 ; C[C@]1(C2CC2)CN(C(=O)Nc2cnc(-n3nccn3)c(Cl)c2)c2cnc3cc(Cl)nn3c21]

The racemic mixture of 2-chloro-N-(5-chloro-6-(2H-1,2,3-triazol-2-yl)pyridin-3-yl)-8-cyclopropyl-8-methyl-7,8-dihydro-6H-pyrazolo[1,5-a]pyrrolo[2,3-e]pyrimidine-6-carboxamide (50.0 mg, 106.1 μmol) was purified by CHIRAL-HPLC (Column: CHIRAL ART Cellulose-SB, 2*25 cm, 5 um; Mobile Phase A:Hex:DCM=3:1(10 mM NH₃-MeOH)—HPLC, Mobile Phase B:EtOH—HPLC; Flow rate: 20 mL/min; isocratic 20% B; 254/220 nm; RT1:12.586; RT2:15.434; Injection Volume: 0.6 ml; Number of Runs: 5). The first eluting isomer was concentrated, lyophilized, and repurified by prep-HPLC to afford Example 3 (14.1 mg, 37%). The second eluting isomer was concentrated, lyophilized, and repurified by prep-HPLC to afford Example 4 (12.8 mg, 34%).

Example 3: ¹H NMR (300 MHz, CDCl₃): 9.38 (s, 1H), 8.61 (s, 1H), 8.41 (s, 1H), 8.00 (s, 2H), 6.75 (s, 2H), 3.77-3.84 (m, 2H), 1.81-1.82 (m, 4H), 0.73-0.76 (m, 1H), 0.69-0.71 (m, 2H), 0.58-0.61 (m, 1H). LC-MS: m/z 470 [M+H]⁺.

Example 4: ¹H NMR (300 MHz, CDCl₃): 9.34 (s, 1H), 8.64 (s, 1H), 8.42 (s, 1H), 8.00 (s, 2H), 6.70 (s, 2H), 3.78-3.84 (m, 2H), 1.81-1.82 (m, 4H), 0.86-0.91 (m, 1H), 0.74-0.78 (m, 2H), 0.72-0.73 (m, 1H). LC-MS: m/z 470 [M+H]⁺.

### Method E1

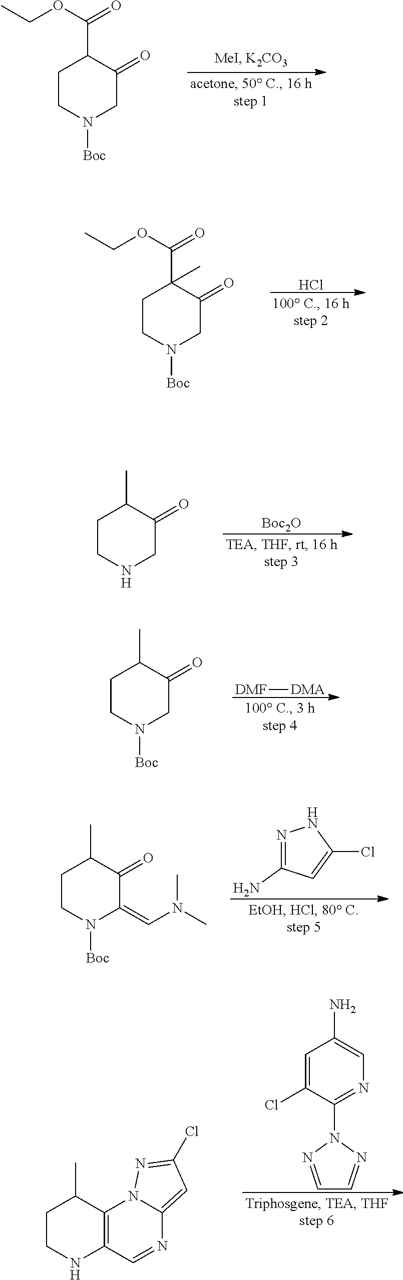

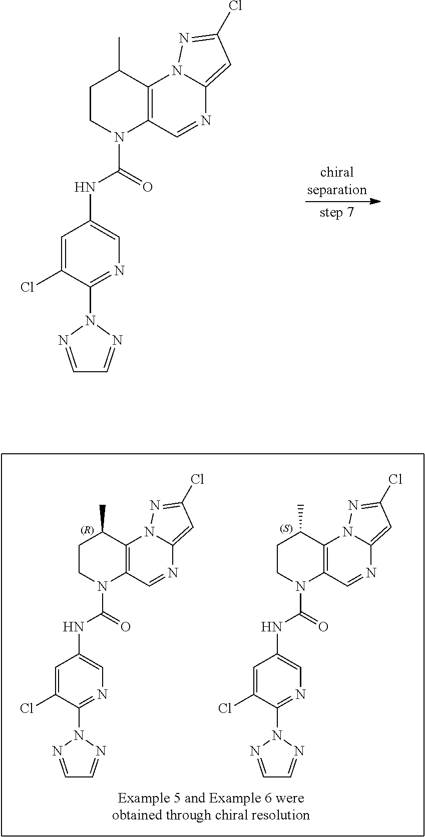

### Examples 5 and 6: Single enantiomers obtained from a racemic mixture containing (R)-2-chloro-N-(5-chloro-6(2H-1,2,3-triazol-2-yl)pyridin-3-yl)-9-methyl-8,9-dihydropyrazolo[1,5-a]pyrido[2,3-e]pyrimidine-6(7H)-carboxamide and (S)-2-chloro-N-(5-chloro-6-(2H-1,2,3-triazol-2-yl)pyridin-3-yl)-9-methyl-8,9-dihydropyrazolo[1,5-a]pyrido[2,3-e]pyrimidine-6(7H)-carboxamide

### Step 1: 1-(tert-butyl) 4-ethyl 4-methyl-3-oxopiperidine-1,4-dicarboxylate

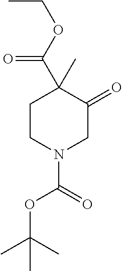 [SMILES: CCOC(=O)C1(C)CCN(C(=O)OC(C)(C)C)CC1=O]

To a stirred solution of 1-tert-butyl 4-ethyl 3-oxopiperidine-1,4-dicarboxylate (20 g, 73 mmol, 1 equiv.) and K₂CO₃ (20.4 g, 146 mmol, 2 equiv.) in acetone (100 mL) was added MeI (20.9 g, 146 mmol, 2 equiv.) at rt under nitrogen. The resulting mixture was stirred for 16 h at 50° C. under nitrogen. The mixture was allowed to cool down to 25° C. The resulting mixture was filtered, and the filter cake was washed with EtOAc (3×50 mL). The filtrate was concentrated under reduced pressure. To the residue was added water (300 mL) and the resulting mixture was extracted with EtOAc (3×200 mL). The combined organic layers were dried over anhydrous Na₂SO₄, filtered and concentrated under reduced pressure to afford 1-tert-butyl 4-ethyl 4-methyl-3-oxopiperidine-1, 4-dicarboxylate (20 g, 91%) as a yellow liquid. ¹H NMR (300 MHz, CDCl₃) δ: 4.08-4.18 (m, 2H), 3.46-3.63 (s, 2H), 2.54-2.62 (m, 2H), 1.71 (s, 1H), 1.61 (s, 1H), 1.47 (s, 9H), 1.36 (s, 3H), 1.19-1.34 (m, 3H). LC-MS: m/z 286 [M+H]⁺.

### Step 2: 4-methylpiperidin-3-one

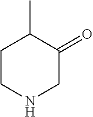

A solution of 1-tert-butyl 4-ethyl 4-methyl-3-oxopiperidine-1,4-dicarboxylate (6 g, 21 mmol, 1 equiv.) in HCl (60 mL) was stirred for 16 h at 100° C. The mixture was allowed to cool down to rt. The resulting mixture was concentrated under reduced pressure to afford 4-methylpiperidin-3-one hydrochloride (6 g, crude) as a yellow oil. LC-MS: m/z 114 [M+H]⁺.

### Step 3: tert-butyl 4-methyl-3-oxopiperidine-1-carboxylate

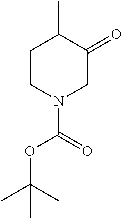

To a stirred solution of 4-methylpiperidin-3-one hydrochloride (6 g, 40 mmol, 1 equiv.) and TEA (12.2 g, 120 mmol, 3 equiv) in THF (100 mL) was added Boc₂O (26.3 g, 120 mmol, 3 equiv.) in portions at rt. The resulting mixture was stirred for 16 h at rt. The reaction was quenched by the addition of water (200 mL). The resulting mixture was extracted with EtOAc (3×100 mL). The combined organic layers were washed with brine (100 mL) and dried over anhydrous Na₂SO₄. After filtration, the filtrate was concentrated under reduced pressure. The residue was purified by silica gel column chromatography, eluted with CH₂Cl₂/PE (10:1) to afford tert-butyl 4-methyl-3-oxopiperidine-1-carboxylate (4.7 g, 55%) as a yellow liquid. ¹H NMR (300 MHz, CDCl₃) δ: 4.07-4.11 (m, 2H), 3.33-3.48 (m, 2H), 2.42-2.47 (m, 1H), 1.60-1.74 (m, 2H), 1.51-1.65 (m, 1H), 1.36 (s, 3H), 1.15 (d, J=6.9 Hz, 6H). LC-MS: m/z 214 [M+H]⁺.

### Step 4: tert-butyl (E)-2-((dimethylamino)methylene)-4-methyl-3-oxopiperidine-1-carboxylate

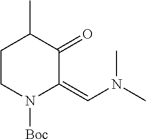 [SMILES: CC1CCN(C(=O)OC(C)(C)C)/C(=C/N(C)C)C1=O]

A solution of tert-butyl 4-methyl-3-oxopiperidine-1-carboxylate (2 g, 9.4 mmol, 1.0 equiv.) in DMF-DMA (10 mL) was stirred for 4 h at 100° C. The mixture was allowed to cool to 25° C. The resulting mixture was concentrated under reduced pressure to afford tert-butyl (2E)-2-[(dimethylamino) methylidene]-4-methyl-3-oxopiperidine-1-carboxylate (2 g, 79%) as a yellow oil. LC-MS: m/z 269 [M+H]⁺.

### Step 5: 2-chloro-9-methyl-6,7,8,9-tetrahydropyrazolo[1,5-a]pyrido[2,3-e]pyrimidine

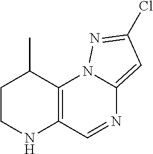

To a stirred solution of tert-butyl (2E)-2-[(dimethylamino)methylidene]-4-methyl-3-oxopiperidine-1-carboxylate (2.0 g, 7.4 mmol, 1.0 equiv.) in EtOH (20 mL) were added 5-chloro-1H-pyrazol-3-amine (0.9 g, 7.4 mmol, 1.0 equiv.) and HCl in 1,4-dioxane (10 mL) at 25° C. The resulting mixture was stirred for 16 h at 80° C. The mixture was allowed to cool down to 25° C. The resulting mixture was concentrated under reduced pressure. A saturated solution of NaHCO₃ (100 mL) was added and the mixture was extracted with EtOAc (3×100 mL). The combined organic layers were dried over anhydrous Na₂SO₄, filtered and concentrated under reduced pressure. The residue was applied on a silica gel column and eluted with PE/EtOAc (1:1) to afford 2-chloro-9-methyl-6,7,8,9-tetrahydropyrazolo[1,5-a]pyrido[2,3-e]pyrimidine (260 mg, 15%) as a yellow oil. ¹H NMR (300 MHz, CDCl₃) δ: 8.18 (s, 1H), 6.64 (s, 1H), 6.02 (s, 1H), 3.37-3.47 (m, 1H), 3.10-3.27 (m, 1H), 1.73-2.03 (m, 2H), 1.35 (d, J=6.9 Hz, 3H). LC-MS: m/z 223 [M+H]⁺.

### Step 6: 2-chloro-N-(5-chloro-6-(2H-1,2,3-triazol-2-yl)pyridin-3-yl)-9-methyl-8,9-dihydropyrazolo [1,5-a]pyrido[2,3-e]pyrimidine-6(7H)-carboxamide

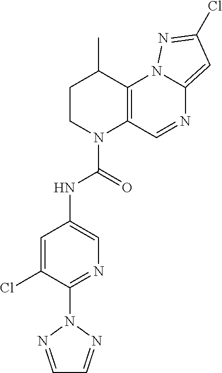 [SMILES: CC1CCN(C(=O)Nc2cnc(-n3nccn3)c(Cl)c2)c2cnc3cc(Cl)nn3c21]

To a stirred solution of 5-chloro-6-(1,2,3-triazol-2-yl)pyridin-3-amine (Method A1 step 2; 242.4 mg, 1.2 mmol, 1.2 equiv.) and TEA (125.4 mg, 1.2 mmol, 1.2 equiv.) in THF (20 mL) was added triphosgene (122.6 mg, 0.4 mmol, 0.4 equiv.) at 0° C. The resulting mixture was stirred for 30 min at 25° C. The solids were filtered out, and to the filtrate was added 2-chloro-9-methyl-6,7,8,9-tetrahydropyrazolo[1,5-a]pyrido[2,3-e]pyrimidine (230 mg, 1.0 mmol, 1.0 equiv.). The resulting mixture was stirred for 16 h at 25° C. The mixture was poured into water (50 mL) and extracted with EtOAc (3×50 mL). The combined organic layers were dried over anhydrous Na₂SO₄, filtered and concentrated under vacuum. The residue was submitted to Prep-HPLC purification. The collected fractions were lyophilized to give 16 mg of 2-chloro-N-(5-chloro-6-(2H-1,2,3-triazol-2-yl)pyridin-3-yl)-9-methyl-8,9-dihydropyrazolo[1,5-a]pyrido[2,3-e]pyrimidine-6(7H)-carboxamide (3% yield) as a racemic mixture. LC-MS: m/z 444 [M+H]⁺.

### Step 7: Separation of enantiomers to obtain (R)-2-chloro-N-(5-chloro-6-(2H-1,2,3-triazol-2-yl)pyridin-3-yl)-9-methyl-8,9-dihydropyrazolo[1,5-a]pyrido[2,3-e]pyrimidine-6(7H)-carboxamide and (S)-2-chloro-N-(5-chloro-6-(2H-1,2,3-triazol-2-yl)pyridin-3-yl)-9-methyl-8,9-dihydropyrazolo[1,5-a]pyrido[2,3-e]pyrimidine-6(7H)-carboxamide

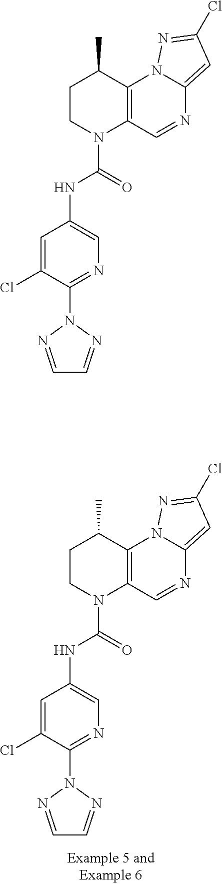 [SMILES: C[C@@H]1CCN(C(=O)Nc2cnc(-n3nccn3)c(Cl)c2)c2cnc3cc(Cl)nn3c21 ; C[C@H]1CCN(C(=O)Nc2cnc(-n3nccn3)c(Cl)c2)c2cnc3cc(Cl)nn3c21]

100 mg of 2-chloro-N-(5-chloro-6-(2H-1,2,3-triazol-2-yl)pyridin-3-yl)-9-methyl-8,9-dihydropyrazolo [1,5-a]pyrido[2,3-e]pyrimidine-6(7H)-carboxamide was submitted to CHIRAL-HPLC purification (Column: CHIRAL ART Cellulose-SB, 2*25 cm, 5 um; Mobile Phase A:Hex:DCM=3:1(10 mM NH₃-MEOH)—HPLC, Mobile Phase B:EtOH—HPLC; Flow rate: 20 mL/min; isocratic 20% B; 254/220 nm; RT1:12.586; RT2:15.434; Injection Volume: 0.6 ml; Number of Runs: 5) to give the first eluting isomer Example 5 (46 mg, 10% yield) and the second eluting isomer Example 6 (45 mg, 9% yield).

Example 5: ¹H NMR (300 MHz, DMSO-d₆) δ: 9.99 (s, 1H), 8.82 (s, 1H), 8.64 (d, J=2.1 Hz, 1H), 8.39 (d, J=2.1 Hz, 1H), 8.17 (s, 2H), 6.90 (s, 1H), 3.98-4.05 (m, 1H), 3.82-3.85 (m, 1H), 3.55-3.61 (m, 1H), 2.20-2.25 (m, 1H), 1.91-1.94 (m, 1H), 1.47 (d, J=6.9 Hz, 3H). LC-MS: m/z 444 [M+H]⁺.

Example 6: ¹H NMR (300 MHz, DMSO-d₆) δ: 9.99 (s, 1H), 8.82 (s, 1H), 8.64 (d, J=2.1 Hz, 1H), 8.39 (d, J=2.1 Hz, 1H), 8.17 (s, 2H), 6.90 (s, 1H), 3.984.05 (m, 1H), 3.82-3.85 (m, 1H), 3.55-3.61 (m, 1H), 2.20-2.25 (m, 1H), 1.91-1.94 (m, 1H), 1.47 (d, J=6.9 Hz, 3H). LC-MS: 444 [M+H]⁺.

### Method F1

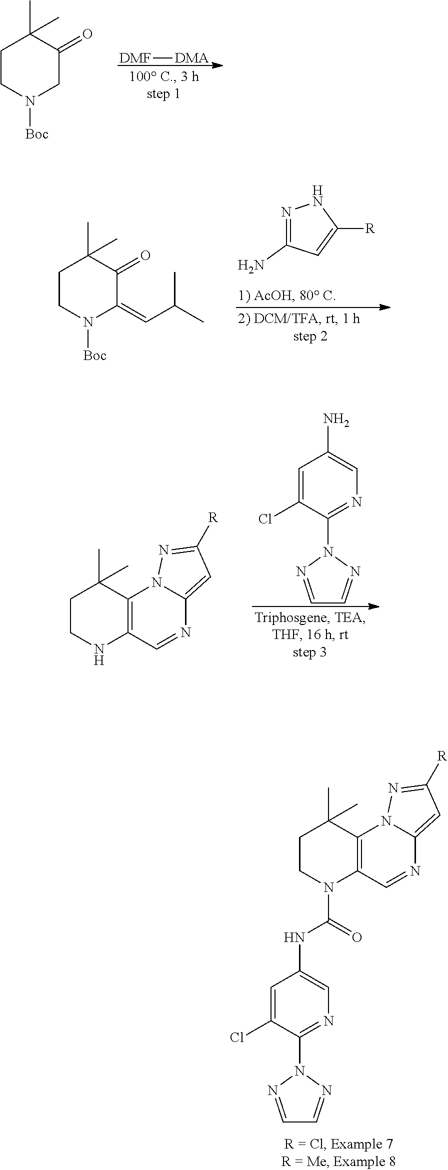

### Example 7: 2-chloro-N-(5-chloro-6-(2H-1,2,3-triazol-2-yl)pyridin-3-yl)-9,9-dimethyl-8,9-dihydropyrazolo[1,5-a]pyrido[2,3-e]pyrimidine-6(7H)-carboxamide

### Step 1: tert-butyl (E)-2-((dimethylamino)methylene)-4,4-dimethyl-3-oxopiperidine-1-carboxylate

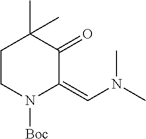 [SMILES: CN(C)/C=C1\C(=O)C(C)(C)CCN1C(=O)OC(C)(C)C]

A solution of tert-butyl 4,4-dimethyl-3-oxo-piperidine-1-carboxylate (5.0 g, 22.0 mmol) in 1,1-dimethoxy-N,N-dimethyl-methanamine (20 ml) was stirred for 3 h at 100° C. under nitrogen atmosphere. The mixture was allowed to cool down to 25° C. The resulting mixture was concentrated under vacuum to afford the product tert-butyl(2E)-2-(dimethylaminomethylene-4,4-dimethyl-3-oxo-piperidine-1-carboxylate (5.0 g, 80% yield) as a brown oil. The crude product was used directly in the next step without further purification. LC-MS: m/z 283 [M+H]⁺.

### Step 2: 2-chloro-9,9-dimethyl-6,7,8,9-tetrahydropyrazolo[1,5-a]pyrido[2,3-e]pyrimidine

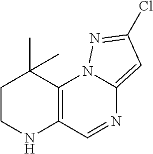 [SMILES: CC1(C)CCNc2cnc3cc(Cl)nn3c21]

A solution of 5-chloro-1H-pyrazol-3-amine (83.2 mg, 708.3 μmol) and tert-butyl (2E)-2-(dimethyl aminomethylene)-4,4-dimethyl-3-oxo-piperidine-1-carboxylate (200 mg, 708.3 μmol) in AcOH (4 mL) was stirred for 3 h at 80° C. under nitrogen atmosphere. The mixture was allowed to cool down to 25° C. and concentrated under vacuum. TFA (0.5 mL) and DCM (2.5 ml) were added. The resulting mixture was stirred for additional 1 h at 25° C. The resulting mixture was concentrated under vacuum and purified by silica gel column chromatography (eluting with 0-50% ethyl acetate in hexane) to afford 2-chloro-9,9-dimethyl-6,7,8,9-tetrahydropyrazolo[1,5-a]pyrido[2,3-e]pyrimidine (80 mg, 48% yield) as a light yellow oil. LC-MS: m/z 237 [M+H]⁺.

### Step 3: 2-chloro-N-(5-chloro-6-(2H-1,2,3-triazol-2-yl)pyridin-3-yl)-9,9-dimethyl-8,9-dihydropyrazolo[1,5-a]pyrido[2,3-e]pyrimidine-6(7H)-carboxamide

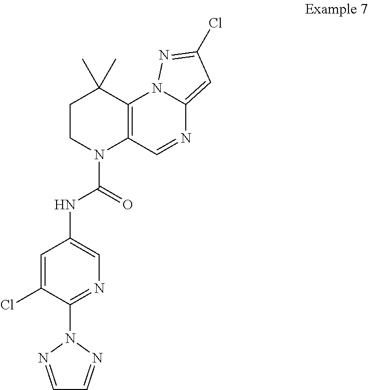 [SMILES: CC1(C)CCN(C(=O)Nc2cnc(-n3nccn3)c(Cl)c2)c2cnc3cc(Cl)nn3c21]

To a stirred solution of 5-chloro-6-(triazol-2-yl)pyridin-3-amine (Method A1 step 2; 69 mg, 354.9 μmol) in THF (6 mL) were added bis(trichloromethyl) carbonate (52 mg, 177.4 μmol) and N,N-diethylethanamine (38 mg, 384.4 μmol, 53.6 μL) in portions at 25° C. The resulting mixture was stirred for 30 min at 25° C. The solids were filtered out. To the filtrate was added 2-chloro-9,9-dimethyl-6,7,8,9-tetrahydropyrazolo[1,5-a]pyrido[2,3-e]pyrimidine (70 mg, 295.7 μmol) in portions. The resulting mixture was stirred overnight at 25° C. Water (50 mL) was added and the mixture was extracted with 3×50 mL of DCM. The organic layers were combined, washed with brine, dried and concentrated under vacuum. The crude product (70 mg) was purified by Prep-HPLC and the collected fractions were lyophilized to afford 2-chloro-N-(5-chloro-6-(2H-1,2,3-triazol-2-yl)pyridin-3-yl)-9,9-dimethyl-8,9-dihydropyrazolo[1,5-a]pyrido[2,3-e]pyrimidine-6(7H)-carboxamide (25 mg, 18% yield) as a white solid. ¹H NMR (400 MHz, DMSO-d₆) δ 9.97 (s, 1H), 8.73 (s, 1H), 8.63 (d, J=2.4 Hz, 1H), 8.40 (d, J=2.4 Hz, 1H), 8.17 (s, 2H), 6.90 (s, 1H), 3.88-3.90 (m, 2H), 2.08 (s, 1H), 1.98-2.00 (m, 2H), 1.65 (s, 6H). LC-MS: m/z 458 [M+H]⁺.

### Example 8: N-(5-chloro-6-(2H-1,2,3-triazol-2-yl)pyridin-3-yl)-2,9,9-trimethyl-8,9-dihydropyrazolo[1,5-a]pyrido[2,3-e]pyrimidine-6(7H)-carboxamide

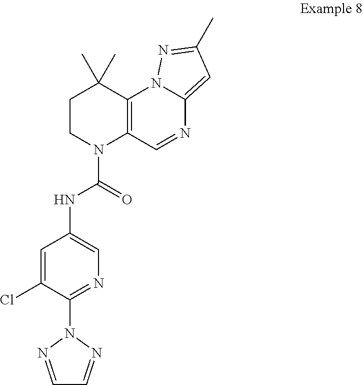 [SMILES: Cc1cc2ncc3c(n2n1)C(C)(C)CCN3C(=O)Nc1cnc(-n2nccn2)c(Cl)c1]

The title compound was prepared according to Method F1 using 5-methyl-1H-pyrazol-3-amine in step 2 (46 mg, 22% yield). ¹H NMR (400 MHz, Methanol-d₄) δ: 8.60 (d, J=2.4 Hz, 1H), 8.54 (s, 1H), 8.44 (d, J=2.4 Hz, 1H), 8.03 (s, 2H), 6.46 (d, J=0.6 Hz, 1H), 3.94-3.97 (m, 2H), 2.51 (s, 3H), 2.07 (dt, J=8.4, 2.8 Hz, 2H), 1.77 (s, 6H). LC-MS: m/z 438 [M+H]⁺.

### Method G1

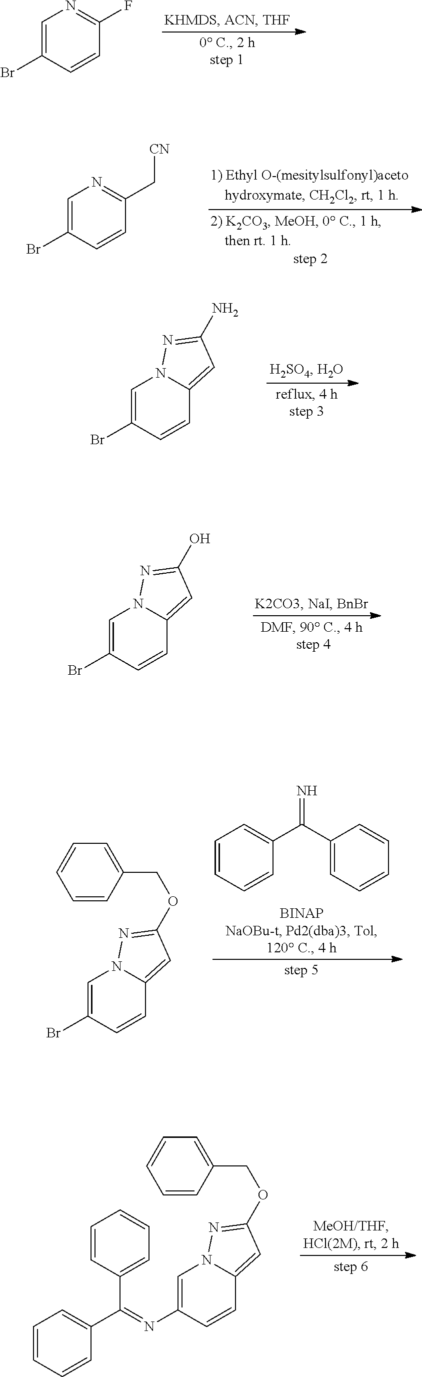

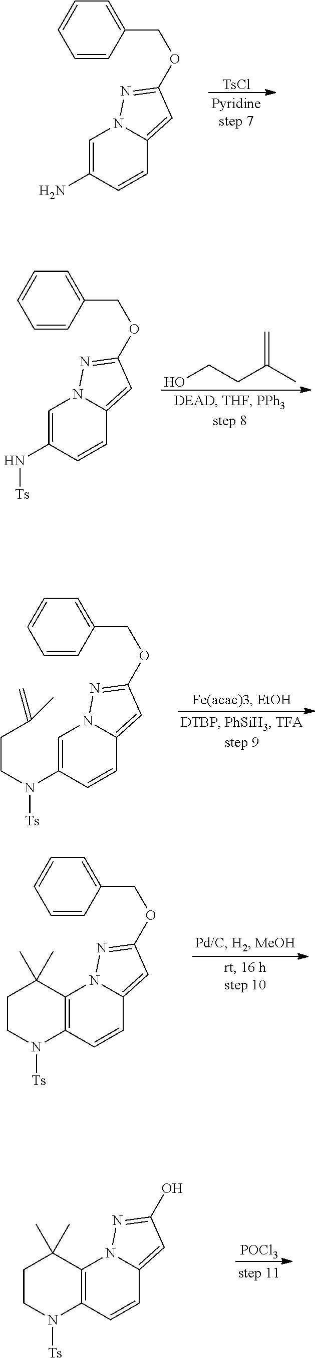

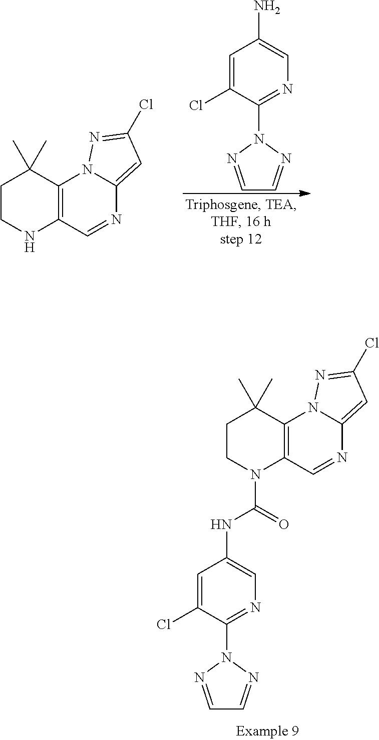 [SMILES: CC1(C)CCNc2cnc3cc(Cl)nn3c21 ; Nc1cnc(-n2nccn2)c(Cl)c1 ; CC1(C)CCN(C(=O)Nc2cnc(-n3nccn3)c(Cl)c2)c2cnc3cc(Cl)nn3c21]

### Example 9: 2-chloro-N-(5-chloro-6-(2H-1,2,3-triazol-2-yl)pyridin-3-yl)-9,9-dimethyl-8,9-dihydropyrazolo[1,5-a][1,5]naphthyridine-6(7H)-carboxamide

### Step 1: 2-(5-bromopyridin-2-yl)acetonitrile

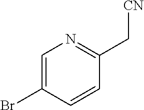

In a 2000-mL round bottom flask, to a solution of potassium bis(trimethylsilyl)azanide (1 M, 852.3 mL) was added dropwise acetonitrile (17.5 g, 426.2 mmol, 22.3 mL) at 0° C. under N₂ atmosphere. The reaction mixture was stirred at 0° C. for 30 min. A solution of 5-bromo-2-fluoro-pyridine (15 g, 85.2 mmol, 8.8 mL) in THF (10 mL) was added dropwise and the mixture was stirred for another 2 h. The reaction mixture was quenched with H₂O/sat. NH₄Cl (1000 mL) and extracted with EtOAc (2×1500 mL). The combined organic extracts were washed with brine (1000 mL), dried over anhydrous Na₂SO₄, and concentrated under vacuum. The residue was purified by flash column chromatography (eluting with 0-50% EtOAc in hexane) to afford 2-(5-bromo-2-pyridyl)acetonitrile (6 g, 30.4 mmol) as a colorless oil. LC-MS: m/z 197 [M+H]⁺.

### Step 2: 6-bromopyrazolo[1,5-a]pyridin-2-amine

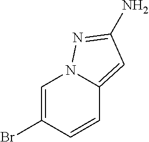 [SMILES: Nc1cc2ccc(Br)cn2n1]

To a stirred solution of ethyl (1E)-N-(2,4,6-trimethyl phenyl)sulfonyloxyethanimidate (13.0 g, 45.7 mmol) in 1,4-dioxane (26 mL) was added perchloric acid (8.7 g, 60.9 mmol, 70% purity) dropwise at 0° C. under N₂. The resulting mixture was stirred for 30 min at 0° C. under nitrogen. Water (60 mL) was added dropwise over 3 min at 0° C. The solid was filtered and the filter cake was dissolved in DCM (240 mL), and the resulting solution was dried over anhydrous sodium sulfate to give a clear solution. This solution was then added dropwise at 0° C. under N₂ over the period of 30 min to a stirred solution of 2-(5-bromo-2-pyridyl)acetonitrile (6 g, 30.4 mmol) in DCM (240 mL). The resulting mixture was stirred for 60 min at 25° C. under nitrogen. It was concentrated under vacuum and diluted with MeOH (160 mL). To this mixture was added tripotassium carbonate (12.6 g, 91.4 mmol, 5.5 mL) in portions at 0° C. and the resulting mixture was stirred for additional 2 h at 25° C. The resulting solution was diluted with 250 ml of water and extracted with EtOAc (3×250 mL). The organic layers were combined, washed with brine, dried and concentrated under vacuum. The residue was applied on a silica gel column and eluted with EtOAc/PE (1/1) to give 6-bromopyrazolo[1,5-a]pyridine-2-amine (2.8 g, 43% yield) as a brown solid. ¹H NMR (400 MHz, DMSO-d₆) δ 8.58 (dt, J=1.8, 0.8 Hz, 1H), 7.24 (dd, J=9.2, 0.8 Hz, 1H), 7.10 (dd, J=9.2, 1.8 Hz, 1H), 5.69 (d, J=0.8 Hz, 1H), 5.40 (s, 2H). LC-MS: m/z 212 [M+H]⁺.

### Step 3: 6-bromopyrazolo[1,5-a]pyridin-2-ol

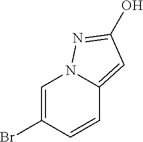 [SMILES: Oc1cc2ccc(Br)cn2n1]

A solution of 6-bromopyrazolo[1,5-a]pyridin-2-amine (2.8 g, 13.20 mmol) in H₂SO₄ (20 mL, 50%) was stirred for 2 h at 100° C. under nitrogen. The mixture was allowed to cool down to rt. The resulting mixture was concentrated under vacuum. Water (50 mL) was added and the mixture was extracted with DCM (3×50 mL). The organic layers were combined, washed with brine, dried and concentrated under vacuum to afford 6-bromopyrazolo[1,5-a]pyridin-2-ol (2.5 g, 89% yield) as a brown solid. The crude product was used in the next step directly without further purification. LC-MS: 213 [M+H]⁺.

### Step 4: 2-(benzyloxy)-6-bromopyrazolo[1,5-a]pyridine

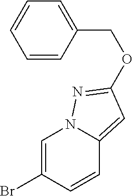 [SMILES: Brc1ccc2cc(OCc3ccccc3)nn2c1]

To a stirred mixture of 6-bromopyrazolo[1,5-a]pyridin-2-ol (2.5 g, 11.7 mmol), potassium carbonate (4.9 g, 35.2 mmol) and sodium iodide (1.8 g, 11.7 mmol) in DMF (15 mL) was added bromomethylbenzene (2.0 g, 11.7 mmol, 1.4 mL) in portions at rt. The resulting mixture was stirred for 16 h at 90° C. The mixture was allowed to cool down to rt, diluted with 150 ml of sodium carbonate (aq.) and extracted with EtOAc (3×150 mL). The organic layers were combined, washed with brine, dried and concentrated under vacuum. The residue was purified by silica gel column chromatography (eluting with DCM/MeOH (10:1)) to afford 2-benzyloxy-6-bromo-pyrazolo[1,5-a]pyridine (2.5 g, 70% yield) as a brown solid. LC-MS: m/z 303 [M+H]⁺.

### Step 5: N-(2-(benzyloxy)pyrazolo[1,5-a]pyridin-6-yl)-1,1-diphenylmethanimine

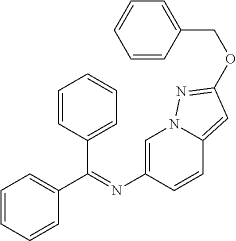 [SMILES: c1ccc(COc2cc3ccc(N=C(c4ccccc4)c4ccccc4)cn3n2)cc1]

To a solution of diphenylmethanimine (1.8 g, 9.9 mmol, 1.7 mL) and 2-benzyloxy-6-bromo-pyrazolo[1,5-a]pyridine (2.5 g, 8.2 mmol) in toluene (20 mL) were added sodium 2-methylpropan-2-olate (1.6 g, 16.5 mmol), Pd₂(dba)₃ (755.2 mg, 824.7 μmol) and benzyl-[1-[2-[benzyl(phenyl)phosphanyl]-1-naphthyl]-2-naphthyl]-phenyl-phosphane (1.1 g, 1.6 mmol). After stirring for 4 h at 120° C. under nitrogen, the resulting mixture was concentrated under reduced pressure. The residue was applied on a silica gel column and eluted with EtOAc/PE (1/5) to give N-(2-benzyloxypyrazolo[1,5-a]pyridin-6-yl)-1,1-diphenyl-methanimine (2.4 g, 72% yield) as a brown solid. LC-MS: m/z 404 [M+H]⁺.

### Step 6: 2-(benzyloxy)pyrazolo[1,5-a]pyridin-6-amine

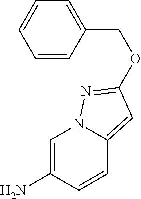 [SMILES: Nc1ccc2cc(OCc3ccccc3)nn2c1]

A mixture of N-(2-benzyloxypyrazolo[1,5-a]pyridin-6-yl)-1,1-diphenyl-methanimine (2.4 g, 6.0 mmol), HCl (2 M, 6.0 mL), THF (10 mL) and MeOH (10 mL) was stirred for 2 h at 25° C. under nitrogen. The mixture was concentrated under vacuum. The residue was purified by silica gel column chromatography (DCM/MeOH=10:1) to afford 2-benzyloxypyrazolo[1,5-a]pyridin-6-amine (1.1 g, 78% yield) as a brown oil. LC-MS: m/z 240 [M+H]⁺.

### Step 7: N-(2-(benzyloxy)pyrazolo[1,5-a]pyridin-6-yl)-4-methylbenzenesulfonamide

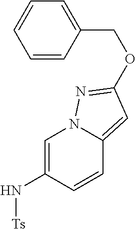 [SMILES: Cc1ccc(S(=O)(=O)Nc2ccc3cc(OCc4ccccc4)nn3c2)cc1]

A solution of 2-benzyloxypyrazolo[1,5-a]pyridin-6-amine (1.1 g, 4.8 mmol) and 4-methylbenzenesulfonyl chloride (999 mg, 5.2 mmol) in pyridine (15 mL) was stirred overnight at rt under nitrogen. The resulting mixture was concentrated under vacuum. To the residue was added water (150 mL) and the pH was adjusted to about 7 by addition of 0.5 M HCl. The mixture was extracted with EtOAc (3×140 mL). The organic layers were combined, washed with brine, dried and concentrated under vacuum. The residue was purified by silica gel column chromatography, using PE/EA (1:1) as eluent to afford N-(2-benzyloxypyrazolo[1,5-a]pyridin-6-yl)-4-methyl-benzenesulfonamide (1.6 g, 85% yield) as an off-white solid. LC-MS: m/z 394 [M+H]⁺.

### Step 8: N-(2-(benzyloxy)pyrazolo[1,5-a]pyridin-6-yl)-4-methyl-N-(3-methylbut-3-en-1-yl)benzenesulfonamide

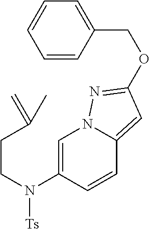 [SMILES: C=C(C)CCN(c1ccc2cc(OCc3ccccc3)nn2c1)S(=O)(=O)c1ccc(C)cc1]

To a stirred solution of 3-methylbut-3-en-1-ol (385 mg, 4.5 mmol), triphenylphosphane (2.1 g, 8.1 mmol) and N-(2-benzyloxypyrazolo[1,5-a]pyridin-6-yl)-4-methyl-benzenesulfonamide (1.6 g, 4.0 mmol) in THF (50 mL) was added isopropyl N-isopropoxycarbonyliminocarbamate (2 M, 4.1 mL) dropwise at 0° C. under nitrogen. The resulting mixture was concentrated under vacuum. The residue was purified by silica gel column chromatography, using PE/EtOAc (5:1) as eluent to afford N-(2-benzyloxypyrazolo[1,5-a]pyridin-6-yl)-4-methyl-N-(3-methylbut-3-enyl)benzenesulfonamide (1.5 g, 80% yield) as a white solid. LC-MS: m/z 462 [M+H]⁺.

### Step 9: 2-(benzyloxy)-9,9-dimethyl-6-tosyl-6,7,8,9-tetrahydropyrazolo[1,5-a][1,5]naphthyridine

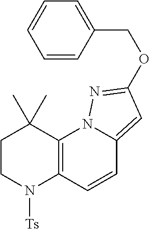 [SMILES: Cc1ccc(S(=O)(=O)N2CCC(C)(C)c3c2ccc2cc(OCc4ccccc4)nn32)cc1]

To a stirred mixture of 2-benzyloxy-N-(3-methylbut-3-enyl)pyrazolo[1,5-a]pyridin-6-amine (300 mg, 976.0 μmol) and ferric (Z)-4-oxopent-2-en-2-olate (172 mg, 488.0 μmol) in EtOH (2 mL) were added phenylsilane (22 mg, 203.3 μmol), 2-tert-butylperoxy-2-methyl-propane (35 mg, 244.0 μmol) and 2,2,2-trifluoroacetic acid (222 mg, 2.0 mmol) in portions at rt under nitrogen, and the mixture was stirred overnight at 60° C. under nitrogen. The mixture was concentrated under vacuum and the residue was purified by silica gel column chromatography, using PE/EtOAc (5:1) as eluent to afford 2-(benzyloxy)-9,9-dimethyl-6-tosyl-6,7,8,9-tetrahydropyrazolo[1,5-a][1,5]naphthyridine (150 mg, 33% yield) as a light yellow solid. LC-MS: m/z 462 [M+H]⁺.

### Step 10: 9,9-dimethyl-6-tosyl-6,7,8,9-tetrahydropyrazolo[1,5-a][1,5]naphthyridin-2-ol

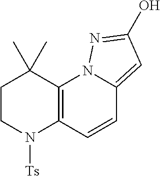 [SMILES: Cc1ccc(S(=O)(=O)N2CCC(C)(C)c3c2ccc2cc(O)nn32)cc1]

To a solution of 2-benzyloxy-9,9-dimethyl-6-(p-tolylsulfonyl)-7,8-dihydropyrazolo[1,5-a][1,5]naphthayridine (300 mg, 649.9 μmol) in MeOH (20 mL) was added Pd/C (10%, 38.5 mg) under nitrogen in a 100 ml round-bottom flask. The mixture was stirred at rt for 16 h under hydrogen atmosphere using a hydrogen balloon, filtered through a celite pad and concentrated under reduced pressure. The residue was dried to afford 9,9-dimethyl-6-tosyl-6,7,8,9-tetrahydropyrazolo[1,5-a][1,5]naphthyridin-2-ol (150 mg, 62% yield) as an off white solid. LC-MS: m/z 372 [M+H]⁺.

### Step 11: 2-chloro-9,9-dimethyl-6,7,8,9-tetrahydropyrazolo[1,5-a][1,5]naphthyridine

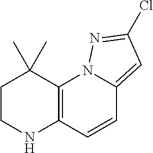 [SMILES: CC1(C)CCNc2ccc3cc(Cl)nn3c21]

Into a 4 mL vial were added 9,9-dimethyl-6-(p-tolylsulfonyl)-7,8-dihydropyrazolo[1,5-a][1,5]naphthyridin-2-ol (100 mg, 269.2 μmol) and POCl₃ (0.8 mL) at rt. The resulting mixture was stirred for 6 h at 145° C. under nitrogen. The reaction mixture was poured onto 50 g of crushed ice. The resulting mixture was extracted with CHCl₃ (3×50 mL). The combined organic layers were washed with brine and dried over anhydrous Na₂SO₄, filtered and concentrated under reduced pressure. The residue was purified by silica gel column chromatography, using CH₂Cl₂/MeOH (10:1) as eluent to afford 2-chloro-9,9-dimethyl-6,7,8,9-tetrahydropyrazolo[1,5-a][1,5]naphthyridine (15 mg, 24% yield) as a brown solid. LC-MS: m/z 236 [M+H]⁺.

### Step 12: 2-chloro-N-(5-chloro-6-(2H-1,2,3-triazol-2-yl)pyridin-3-yl)-9,9-dimethyl-8,9-dihydropyrazolo[1,5-a][1,5]naphthyridine-6(7H)-carboxamide

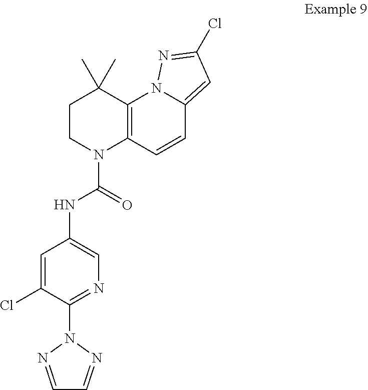 [SMILES: CC1(C)CCN(C(=O)Nc2cnc(-n3nccn3)c(Cl)c2)c2ccc3cc(Cl)nn3c21]

To a stirred solution of 5-chloro-6-(triazol-2-yl)pyridin-3-amine (Method A1 step 2; 15.9 mg, 81.5 μmol) in THF (3 mL) were added bis(trichloromethyl) carbonate (12 mg, 40.7 μmol) and N,N-diethylethanamine (10 mg, 101.8 μmol, 14.2 μL) in portions at rt. The resulting mixture was stirred for 30 min at rt. The solid was filtered out. To the filtrate was added 2-chloro-9,9-dimethyl-7,8-dihydro-6H-pyrazolo[1,5-a][1,5]naphthyridine (16 mg, 67.9 μmol) in portions and the mixture was stirred overnight at rt. Water (20 ml) was added and the resulting mixture was extracted with DCM (3×20 mL). The organic layers were combined, washed with brine, dried and concentrated under vacuum. The crude product (20 mg) was purified by prep-HPLC to afford 2-chloro-N-(5-chloro-6-(2H-1,2,3-triazol-2-yl)pyridin-3-yl)-9,9-dimethyl-8,9-dihydropyrazolo[1,5-a][1,5]naphthyridine-6(7H)-carboxamide (7.8 mg, 22% yield) as a white solid. ¹H NMR (400 MHz, Methanol-d₄) δ 8.55 (d, J=2.0 Hz, 1H), 8.39 (d, J=2.4 Hz, 1H), 8.00 (s, 2H), 7.42 (d, J=9.6 Hz, 1H), 7.34 (d, J=9.6 Hz, 1H), 6.52 (s, 1H), 3.88-3.91 (m, 2H), 2.02-2.05 (m, 2H), 1.70 (d, J=9.6, 1H). LC-MS: m/z 457 [M+H]⁺.

### Method H1

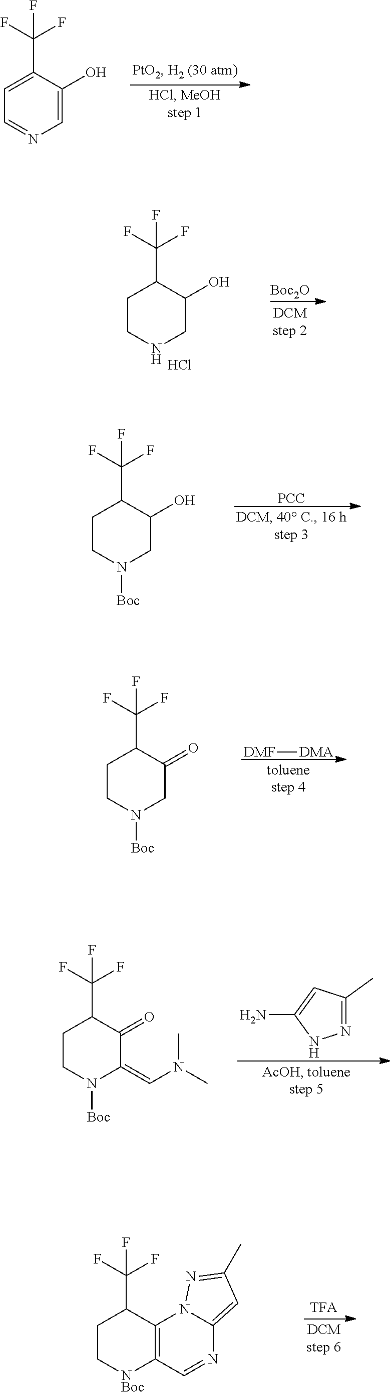

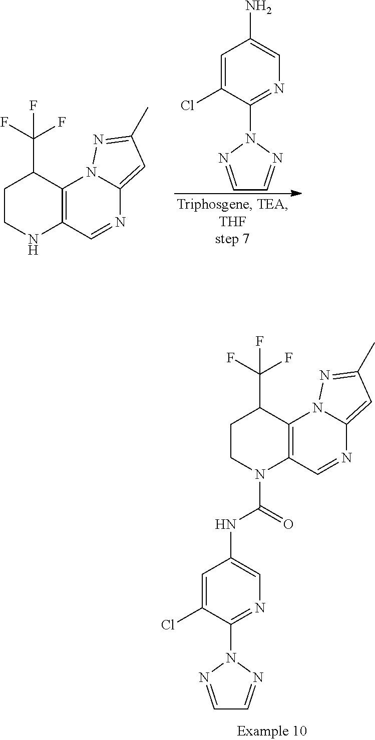

### Example 10: N-(5-chloro-6-(2H-1,2,3-triazol-2-yl)pyridin-3-yl)-2-methyl-9-(trifluoromethyl)-8,9-dihydropyrazolo[1,5-a]pyrido[2,3-e]pyrimidine-6(7H)-carboxamide

### Step 1: 4-(trifluoromethyl) piperidin-3-ol hydrochloride

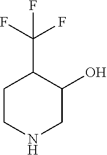 [SMILES: OC1CNCCC1C(F)(F)F]

To a 500 mL pressure tank reactor was added 4-(trifluoromethyl) pyridin-3-ol (9 g, 55.2 mmol) in MeOH (300 mL). PtO₂ (1.4 g) and HCl (9 mL) were added and the reaction mixture was stirred under hydrogen (30 atm) for 48 h at 50° C. The reaction mixture was cool to r, filtered, and concentrated under vacuum to give 4-(trifluoromethyl)piperidin-3-ol hydrochloride (11 g, crude). LC-MS: m/z 170 [M+H]⁺.

### Step 2: tert-butyl 3-hydroxy-4-(trifluoromethyl) piperidine-1-carboxylate

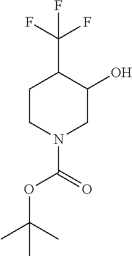 [SMILES: CC(C)(C)OC(=O)N1CCC(C(F)(F)F)C(O)C1]

To a solution of 4-(trifluoromethyl) piperidin-3-ol hydrochloride (11 g, 53.5 mmol) in DCM (200 mL) were added Et₃N (22 g, 214 mmol, 29.8 mL) and Boc₂O (23.3 g, 107 mmol, 24.6 mL). The reaction mixture was stirred for 16 h at rt. The solvent was removed under vacuum and the residue was applied onto a silica gel column and eluted with EtOAc/PE (1:2) to afford tert-butyl 3-hydroxy-4-(trifluoromethyl)piperidine-1-carboxylate (14 g, 41.6 mmol, 78% yield). ¹H NMR (300 MHz, DMSO-d₆) δ: 5.03 (s, 1H), 3.88-4.01 (m, 4H), 2.74-2.86 (m, 2H), 1.74-1.87 (m, 2H), 1.40 (s, 9H). LC-MS: m/z 270 [M+H]⁺.

### Step 3: tert-butyl 3-oxo-4-(trifluoromethyl)piperidine-1-carboxylate

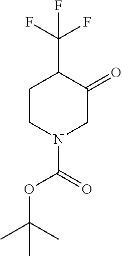 [SMILES: CC(C)(C)OC(=O)N1CCC(C(F)(F)F)C(=O)C1]

To a solution of tert-butyl 3-hydroxy-4-(trifluoromethyl)piperidine-1-carboxylate (7 g, 26.0 mmol) in DCM (200 mL) were added PCC (56 g, 260.0 mmol, 79.1 μL) and silica gel (10 g). The reaction mixture was stirred for 48 h at 40° C. The solid was filtered out and the filtrate was concentrated under vacuum. The crude product was applied onto a silica gel column and eluted with EtOAc/PE (1:3) to afford tert-butyl 3-oxo-4-(trifluoromethyl) piperidine-1-carboxylate (800 mg, 2.4 mmol, 9% yield). ¹H NMR (300 MHz, DMSO-d₆): δ 4.07-4.19 (m, 3H), 3.15-3.26 (m, 2H), 2.04-2.11 (m, 2H), 1.40 (s, 9H). LC-MS: m/z 268.0 [M+H]⁺.

### Step 4: tert-butyl-2-((dimethylamino)methylene)-3-oxo-4-(trifluoromethyl)piperidine-1-carboxylate

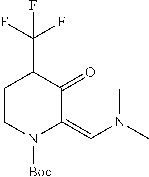 [SMILES: CN(C)/C=C1\C(=O)C(C(F)(F)F)CCN1C(=O)OC(C)(C)C]

To a solution of tert-butyl 3-oxo-4-(trifluoromethyl) piperidine-1-carboxylate (500 mg, 1.9 mmol) in toluene (15 mL) was added DMF-DMA (1.1 g, 9.4 mmol). The reaction mixture was stirred for 1 h at 40° C. and allowed to cool down to rt. The reaction mixture was concentrated to give tert-butyl-2-((dimethylamino)methylene)-3-oxo-4-(trifluoromethyl)piperidine-1-carboxylate (500 mg, crude). LC-MS: m/z 323 [M+H]⁺.

### Step 5: tert-butyl 2-methyl-9-(trifluoromethyl)-8, 9-dihydropyrazolo[1,5-a]pyrido[2,3-e]pyrimidine-6(7H)-carboxylate

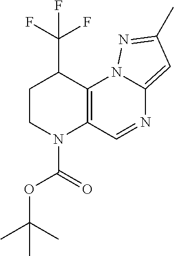 [SMILES: Cc1cc2ncc3c(n2n1)C(C(F)(F)F)CCN3C(=O)OC(C)(C)C]

To a solution of tert-butyl-2-((dimethylamino) methylene)-3-oxo-4-(trifluoromethyl)piperidine-1-carboxylate (100 mg, 310.2 μmol) in toluene (5 mL) were added 3-methyl-1H-pyrazol-5-amine (54 mg, 558.4 μmol) and AcOH (0.5 mL). The reaction mixture was stirred for 2 h at 90° C. The mixture was allowed to cool down to rt. The reaction mixture was concentrated under reduced pressure. To the residue was added water (50 mL) and the pH was adjusted to 6-7 with sodium bicarbonate (sat., aq.). The resulting solution was extracted with EtOAc (50 mL×3). The combined organic layers were dried over anhydrous sodium sulfate and concentrated under vacuum. The residue was applied onto a silica gel column and eluted with EtOAc/PE (1:3) to get tert-butyl 2-methyl-9-(trifluoromethyl)-8,9-dihydropyrazolo[1,5-a]pyrido[2,3-e]pyrimidine-6(7H)-carboxylate (60 mg, 54% yield). ¹H NMR (300 MHz, CDCl₃) δ: 8.81 (s, 1H), 6.52 (s, 1H), 4.80-4.88 (m, 1H), 3.74-3.86 (m, 2H), 2.53 (s, 3H), 2.00-2.13 (m, 2H), 1.28 (s, 9H). LC-MS: m/z 357 [M+H]⁺.

### Step 6: 2-methyl-9-(trifluoromethyl)-6,7,8,9-tetrahydropyrazolo[1,5-a]pyrido[2,3-e]pyrimidine

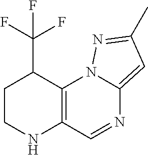 [SMILES: Cc1cc2ncc3c(n2n1)C(C(F)(F)F)CCN3]

To a solution of tert-butyl 2-methyl-9-(trifluoromethyl)-8,9-dihydropyrazolo[1,5-a]pyrido [2,3-e] pyrimidine-6(7H)-carboxylate (40 mg, 111.1 μmol) in CH₂Cl₂ (2 mL) was added TFA (0.5 mL). The reaction was stirred at rt for 1 h. After removal of the solvent, the residue was added NaHCO₃ (30 mL) and extracted with CH₂Cl₂ (3×30 mL). The combined organic layers were washed with brine, dried over sodium sulfate and concentrated in vacuum. The residue was purified by flash chromatography EtOAc/PE (1:1) to afford 2-methyl-9-(trifluoromethyl)-6,7,8,9-tetrahydropyrazolo [1,5-a] pyrido[2,3-e]pyrimidine (8 mg, 28% yield) as a yellow solid. LC-MS: m/z 257.0 [M+H]⁺.

### Step 7: N-(5-chloro-6-(2H-1,2,3-triazol-2-yl)pyridin-3-yl)-2-methyl-9-(trifluoromethyl)-8,9-dihydropyrazolo[1,5-a]pyrido[2,3-e]pyrimidine-6(7H)-carboxamide

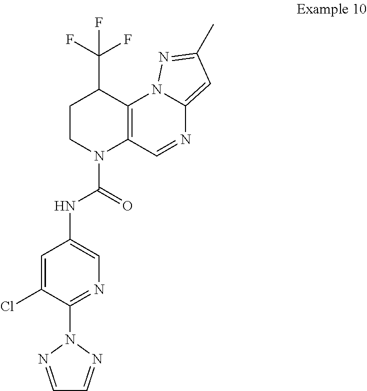 [SMILES: Cc1cc2ncc3c(n2n1)C(C(F)(F)F)CCN3C(=O)Nc1cnc(-n2nccn2)c(Cl)c1]

To a solution of 5-chloro-6-(triazol-2-yl)pyridin-3-amine (Method A1 step 2; 7 mg, 37.5 μmol) in THF (1 mL) were added bis(trichloromethyl) carbonate (6 mg, 21.9 μmol) and N, N-diethylethanamine (9 mg, 93.7 μmol, 13.1 L) at rt. The reaction mixture was stirred at rt for 30 min. The solid was filtered out. To the filtrate was added 2-methyl-9-(trifluoromethyl)-6,7,8,9-tetrahydropyrazolo [1,5-a]pyrido[2,3-e]pyrimidine (8 mg, 31.2 μmol) and the reaction mixture was stirred at rt for 16 h. Water (50 mL) was added and the mixture was extracted with 3×50 mL of EtOAc. The organic layers were combined, dried and concentrated under vacuum. The residue was purified by HPLC to give N-(5-chloro-6-(2H-1,2,3-triazol-2-yl)pyridin-3-yl)-2-methyl-9-(trifluoromethyl)-8,9-dihydropyrazolo[1,5-a]pyrido[2,3-e]pyrimidine-6(7H)-carboxamide (2.4 mg, 16% yield) as a racemic mixture. ¹H NMR (400 MHz, Methanol-d₄) δ: 8.76 (s, 1H), 8.62-8.61 (m, 1H), 8.44-8.45 (m, 1H), 8.01 (s, 2H), 6.56 (s, 1H), 4.86-4.89 (m, 1H), 4.06-4.12 (m, 1H), 3.82-3.89 (m, 1H), 2.65-2.73 (m, 1H), 2.51 (s, 3H), 2.42-2.49 (m, 1H). LC-MS: m/z 478 [M+H]⁺.

### Method I1

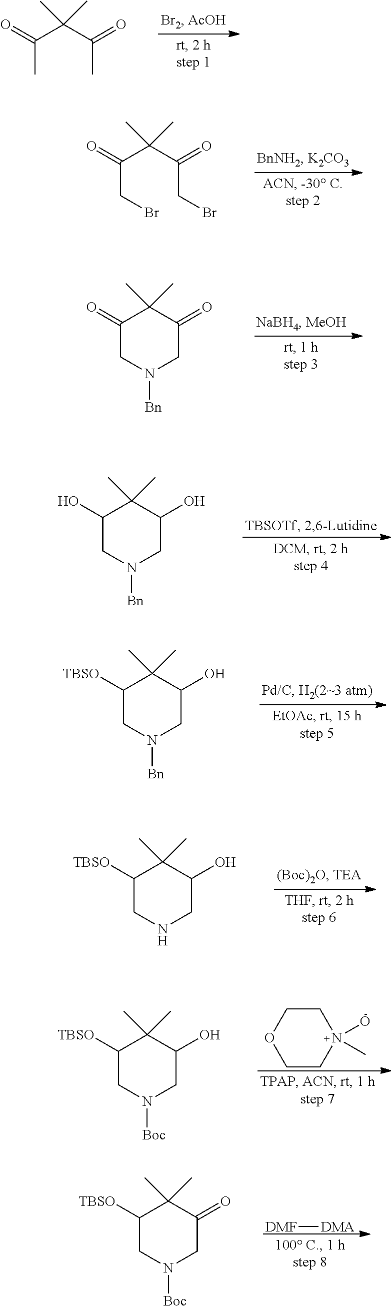

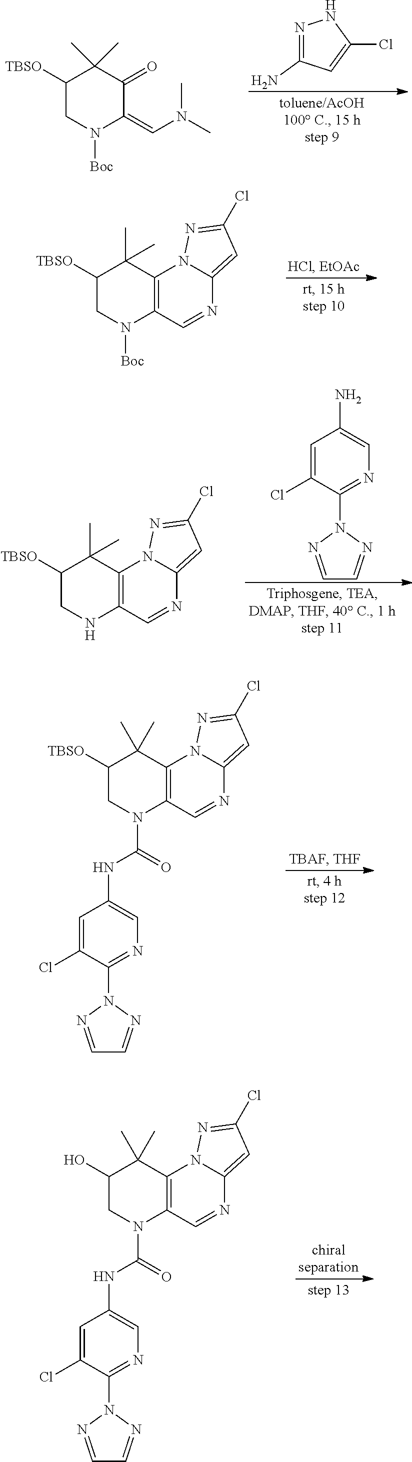

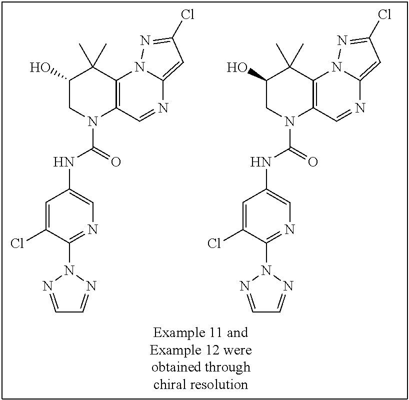 [SMILES: CC1(C)c2c(cnc3cc(Cl)nn23)N(C(=O)Nc2cnc(-n3nccn3)c(Cl)c2)C[C@@H]1O ; CC1(C)c2c(cnc3cc(Cl)nn23)N(C(=O)Nc2cnc(-n3nccn3)c(Cl)c2)C[C@H]1O]

### Examples 11 and 12: Single enantiomers obtained from a racemic mixture containing (R)-2-chloro-N-(5-chloro-6-(2H-1,2,3-triazol-2-yl)pyridin-3-yl)-8-hydroxy-9,9-dimethyl-8,9-dihydropyrazolo[1,5-a]pyrido[2,3-e]pyrimidine-6(7H)-carboxamide and (S)-2-chloro-N-(5-chloro-6(2H-1,2,3-triazol-2-yl)pyridin-3-yl)-8-hydroxy-9,9-dimethyl-8,9-dihydropyrazolo[1,5-a]pyrido[2,3-e]pyrimidine-6(7H)-carboxamide

### Step 1: 1,5-dibromo-3,3-dimethylpentane-2,4-dione

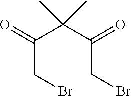

A solution of bromine (12.5 g, 78.0 mmol) in AcOH (30 mL) was added to a solution of 3,3-dimethylpentane-2,4-dione (5.0 g, 39.0 mmol) in AcOH (150 mL) at 10° C. within 1 h. The reaction mixture was stirred at 25° C. for 2 h. AcOK (11.5 g, 117.0 mmol) was added, followed by 150 mL of water, and the mixture was extracted with 200 mL of tert-butyl methyl ether. The combined organic phases were washed with water (3×200 mL), saturated aqueous Na₂S₂O₃ (2×200 mL) and brine (2×200 mL). The resulting solution was dried over anhydrous Na₂SO₄, and all volatiles were removed under reduced pressure to afford 1,5-dibromo-3,3-dimethylpentane-2,4-dione (7 g, 63% yield) as a brown oil. ¹H NMR (300 MHz, CDCl₃-d) δ 4.14 (s, 4H), 1.56 (s, 6H).

### Step 2: 1-benzyl-4,4-dimethylpiperidine-3,5-dione

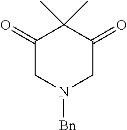 [SMILES: CC1(C)C(=O)CN(Cc2ccccc2)CC1=O]

To a mixture of 1,5-dibromo-3,3-dimethylpentane-2,4-dione (1.0 g, 3.5 mmol) and K₂CO₃ (966 mg, 7.0 mmol) in ACN (50 mL) was added phenylmethanamine (300 mg, 2.8 mmol, in 2 mL ACN) dropwise at −30° C. The reaction mixture was stirred for 30 min at −30° C. and then for 2 h at 25° C. The reaction mixture was concentrated under reduced pressure. The residue was quenched with water (100 mL) and extracted with EtOAc (3×100 mL). The combined organic layers were washed with brine (2×200 mL), dried over anhydrous Na₂SO₄ and concentrated under vacuum. The crude product was purified by reverse phase HPLC. The collected fractions were combined and concentrated under vacuum to afford 1-benzyl-4,4-dimethylpiperidine-3,5-dione (340 mg, 42% yield) as a yellow oil. ¹H NMR (300 MHz, CDCl₃-d) δ 7.29-7.40 (m, 5H), 3.67 (s, 2H), 3.35 (s, 4H), 1.47 (s, 6H); LC-MS: m/z 232 [M+H]⁺.

### Step 3: 1-benzyl-4,4-dimethylpiperidine-3,5-diol

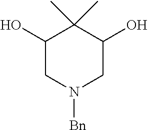 [SMILES: CC1(C)C(O)CN(Cc2ccccc2)CC1O]

To a solution of 1-benzyl-4,4-dimethylpiperidine-3,5-dione (2.5 g, 10.8 mmol) in MeOH (50 mL) was added NaBH₄ (613 mg, 16.2 mmol) in several portions. The reaction mixture was stirred for 1 h at 25° C. The mixture was concentrated under vacuum. The residue was dissolved in EtOAc (200 mL). The mixture was washed with water (3×150 mL), dried over anhydrous Na₂SO₄ and concentrated to afford 1-benzyl-4,4-dimethylpiperidine-3,5-diol (2.3 g, 90% yield) as a yellow solid. LC-MS: m/z 236 [M+H]⁺.

### Step 4: 1-benzyl-5-((tert-butyldimethylsilyl)oxy)-4,4-dimethylpiperidin-3-ol

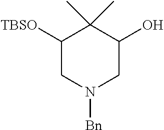 [SMILES: CC1(C)C(O)CN(Cc2ccccc2)CC1O[Si](C)(C)C(C)(C)C]

To a mixture of 1-benzyl-4,4-dimethylpiperidine-3,5-diol (2.1 g, 8.9 mmol) and 2,6-dimethylpyridine (2.4 g, 22.3 mmol) in DCM (100 mL) was added [tert-butyl(dimethyl)silyl]trifluoromethanesulfonate (2.6 g, 9.8 mmol) dropwise at 0° C. The reaction mixture was stirred for 2 h at 25° C. The mixture was concentrated. The residue was purified by reverse phase HPLC. The collected fractions were combined and concentrated under vacuum to afford 1-benzyl-5-((tert-butyldimethylsilyl)oxy)-4,4-dimethylpiperidin-3-ol (710 mg, 23% yield) as a light yellow solid. ¹H NMR (400 MHz, CDCl₃-d) δ 7.32-7.39 (m, 5H), 3.93-4.01 (m, 2H), 7.75-3.78 (m, 1H), 3.47-3.49 (m, 1H), 2.89-2.98 (m, 2H), 2.70-2.73 (m, 1H), 2.29-2.35 (m, 1H), 1.08 (s, 3H), 0.85 (s, 9H), 0.82 (s, 3H), 0.05 (s, 3H), 0.02 (s, 3H). LC-MS: m/z 350 [M+H]⁺.

### Step 5: 5-((tert-butyldimethylsilyl)oxy)-4,4-dimethylpiperidin-3-ol

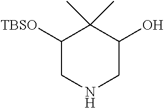 [SMILES: CC1(C)C(O)CNCC1O[Si](C)(C)C(C)(C)C]

To a mixture of 1-benzyl-5-((tert-butyldimethylsilyl)oxy)-4,4-dimethylpiperidin-3-ol (710 mg, 2.0 mmol) in EtOAc (50 mL) was added Pd/C (10%, 700 mg) at 25° C. The flask was evacuated and flushed three times with nitrogen, followed by flushing with hydrogen. The mixture was stirred for 15 h at room temperature under an atmosphere of hydrogen (balloon). The solids were filtrated out and the filtrate was concentrated to afford 5-((tert-butyldimethylsilyl)oxy)-4,4-dimethylpiperidin-3-ol (600 mg, crude) as a yellow solid. ¹H NMR (400 MHz, CDCl₃-d) δ 3.55-3.59 (m, 1H), 3.45-3.48 (m, 1H), 2.94-2.99 (m, 1H), 2.81-2.86 (s, 1H), 2.54-2.71 (m, 2H), 0.94 (s, 3H), 0.93 (s, 3H), 0.89 (s, 9H), 0.06 (s, 3H), 0.03 (s, 3H); LC-MS: m/z 260 [M+H]⁺.

### Step 6: tert-butyl 3-((tert-butyldimethylsilyl)oxy)-5-hydroxy-4,4-dimethylpiperidine-1-carboxylate

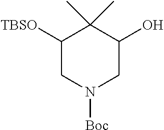 [SMILES: CC(C)(C)OC(=O)N1CC(O)C(C)(C)C(O[Si](C)(C)C(C)(C)C)C1]

To a mixture of 5-((tert-butyldimethylsilyl)oxy)-4,4-dimethylpiperidin-3-ol (600 mg, 2.3 mmol) in THF (100 mL) were added TEA (1.2 g, 11.7 mmol) and tert-butoxycarbonyl tert-butyl carbonate (763 mg, 3.5 mmol) at 25° C. The reaction mixture was stirred for 2 h at 25° C. The mixture was concentrated. The residue was applied onto a silica gel column and eluting with EtOAc/PE (1:2) to afford tert-butyl 3-((tert-butyldimethylsilyl)oxy)-5-hydroxy-4,4-dimethylpiperidine-1-carboxylate (640 mg, 88% yield in two steps) as a white solid. ¹H NMR (300 MHz, CDCl₃-d) δ 3.59-3.70 (m, 4H), 3.38-3.45 (m, 1H), 3.00-3.06 (m, 1H), 1.48 (s, 9H), 1.03 (s, 3H), 0.96 (s, 3H), 0.92 (s, 9H), 0.12 (s, 3H), 0.09 (s, 3H); LC-MS: m/z 360 [M+H]⁺.

### Step 7: tert-butyl 3-((tert-butyldimethylsilyl)oxy)-4,4-dimethyl-5-oxopiperidine-1-carboxylate

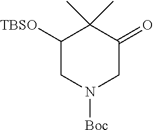 [SMILES: CC(C)(C)OC(=O)N1CC(=O)C(C)(C)C(O[Si](C)(C)C(C)(C)C)C1]

To a mixture of tert-butyl 3-((tert-butyldimethylsilyl)oxy)-5-hydroxy-4,4-dimethylpiperidine-1-carboxylate (650 mg, 1.8 mmol) and TPAP (32 mg, 90.4 μmol) in ACN (10 mL) was added 4-methyl-4-oxido-morpholin-4-ium (275 mg, 2.4 mmol) at 25° C. The reaction mixture was stirred for 1 h at 25° C. The mixture was concentrated under reduced pressure. The residue was applied onto a silica gel column and eluting with EtOAc/PE (1:4) to afford tert-butyl 3-((tert-butyldimethylsilyl)oxy)-4,4-dimethyl-5-oxopiperidine-1-carboxylate (420 mg, 65% yield) as a white solid. ¹H NMR (400 MHz, CDCl₃-d) δ 4.16-4.25 (m, 1H), 3.73-3.91 (m, 3H), 3.50-3.53 (m, 1H), 1.46 (s, 9H), 1.26 (s, 3H), 1.25 (s, 3H), 0.86 (s, 9H), 0.10 (s, 3H), 0.06 (s, 3H). LC-MS: m/z 358 [M+H]⁺.

### Step 8: tert-butyl (E)-5-((tert-butyldimethylsilyl)oxy)-2-((dimethylamino)methylene)-4,4-dimethyl-3-oxopiperidine-1-carboxylate

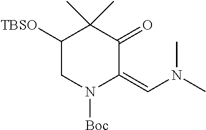 [SMILES: CN(C)/C=C1\C(=O)C(C)(C)C(O[Si](C)(C)C(C)(C)C)CN1C(=O)OC(C)(C)C]

A mixture of tert-butyl 3-((tert-butyldimethylsilyl)oxy)-4,4-dimethyl-5-oxopiperidine-1-carboxylate (420 mg, 1.2 mmol) in DMF-DMA (10 mL) was stirred for 1 h at 100° C. After cooled to 25° C., the mixture was concentrated to afford tert-butyl (E)-5-((tert-butyldimethylsilyl)oxy)-2-((dimethylamino)methylene)-4,4-dimethyl-3-oxopiperidine-1-carboxylate (500 mg, crude) as a yellow oil. The crude product was used in next step without further purification. LC-MS: m/z 413 [M+H]⁺.

### Step 9: tert-butyl 8-((tert-butyldimethylsilyl)oxy)-2-chloro-9,9-dimethyl-8,9-dihydropyrazolo [1,5-a]pyrido[2,3-e]pyrimidine-6(7H)-carboxylate

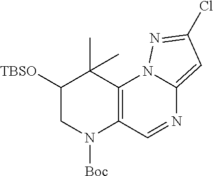 [SMILES: CC(C)(C)OC(=O)N1CC(O[Si](C)(C)C(C)(C)C)C(C)(C)c2c1cnc1cc(Cl)nn21]

To a mixture of tert-butyl (E)-5-((tert-butyldimethylsilyl)oxy)-2-((dimethylamino) methylene)-4,4-dimethyl-3-oxopiperidine-1-carboxylate (500 mg, 1.2 mmol) and 5-chloro-1H-pyrazol-3-amine (142 mg, 1.2 mmol) in toluene (10 mL) was added AcOH (1 mL) at 25° C. The reaction mixture was stirred for 15 h at 100° C. After cooled to 25° C., the mixture was concentrated under vacuum. The residue was dissolved in EtOAc (200 mL). The mixture was washed with saturated aqueous NaHCO₃ (3×150 mL), dried over anhydrous Na₂SO₄ and concentrated. The residue was applied onto a silica gel column and eluted with EtOAc/PE (1:4) to afford tert-butyl 8-((tert-butyldimethylsilyl)oxy)-2-chloro-9,9-dimethyl-8,9-dihydropyrazolo [1,5-a]pyrido[2,3-e]pyrimidine-6(7H)-carboxylate (230 mg, 42% yield over two steps) as a white solid. ¹H NMR (300 MHz, CDCl₃-d) δ 8.80 (s, 1H), 6.59 (s, 1H), 3.80-3.92 (m, 1H), 2.66-2.76 (m, 2H), 1.70 (s, 3H), 1.65 (s, 3H), 1.57 (s, 9H), 0.96 (s, 9H), 0.22 (s, 3H), 0.19 (s, 3H); LC-MS: m/z 467 [M+H]⁺.

### Step 10: 8-((tert-butyldimethylsilyl)oxy)-2-chloro-9,9-dimethyl-6,7,8,9-tetrahydropyrazolo[1,5-a]pyrido[2,3-e]pyrimidine

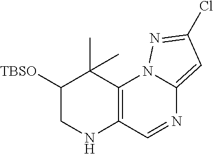 [SMILES: CC1(C)c2c(cnc3cc(Cl)nn23)NCC1O[Si](C)(C)C(C)(C)C]

To a mixture of tert-butyl 8-((tert-butyldimethylsilyl)oxy)-2-chloro-9,9-dimethyl-8,9-dihydropyrazolo [1,5-a]pyrido[2,3-e]pyrimidine-6(7H)-carboxylate (200 mg, 428 μmol) in EtOAc (15 mL) was added HCl (4 M in EtOAc, 5 mL) at 25° C. The reaction mixture was stirred for 15 h. The mixture was concentrated. The residue was dissolved in ethyl acetate (50 mL), washed with sodium carbonate (50 mL, aq., sat.) and brine (50 ML). The resulting solution was dried over anhydrous Na₂SO₄ and concentrated under vacuum. This resulted in 8-((tert-butyldimethylsilyl)oxy)-2-chloro-9,9-dimethyl-6,7,8,9-tetrahydropyrazolo[1,5-a]pyrido[2,3-e]pyrimidine (120 mg, 76% yield) as a yellow solid. ¹H NMR (300 MHz, CDCl₃-d) δ 8.19 (s, 1H), 6.65 (s, 1H), 6.02 (s, 1H), 3.76-3.82 (m, 1H), 3.14-3.19 (m, 1H), 3.04-3.09 (m, 1H), 1.52 (s, 3H), 1.49 (s, 3H), 0.90 (s, 9H), 0.13 (s, 3H), 0.06 (s, 3H); LC-MS: m/z 367 [M+H]⁺.

### Step 11: 8-((tert-butyldimethylsilyl)oxy)-2-chloro-N-(5-chloro-6-(2H-1,2,3-triazol-2-yl)pyridin-3-yl)-9,9-dimethyl-8,9-dihydropyrazolo[1,5-a]pyrido[2,3-e]pyrimidine-6(7H)-carboxamide

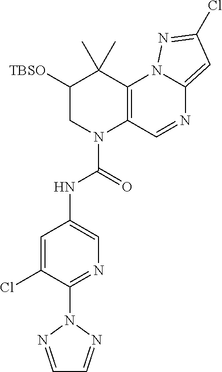 [SMILES: CC1(C)c2c(cnc3cc(Cl)nn23)N(C(=O)Nc2cnc(-n3nccn3)c(Cl)c2)CC1O[Si](C)(C)C(C)(C)C]

To a mixture of 5-chloro-6-(triazol-2-yl)pyridin-3-amine (Method A1 step 2; 64 mg, 327.0 μmol) in THF (1 mL) were added triphosgene (48 mg, 163.5 μmol) and TEA (41 mg, 408.7 μmol) at 25° C. The resulting mixture was stirred for 1 h at 25° C. and then filtered. The resulting filtrate was added to a solution of 8-((tert-butyldimethylsilyl)oxy)-2-chloro-9,9-dimethyl-6,7,8,9-tetrahydropyrazolo[1,5-a]pyrido[2,3-e]pyrimidine (100 mg, 272.5 μmol) in THF (1 mL). To this solution was then added TEA (276 mg, 2.7 mmol) and N,N-dimethylpyridin-4-amine (66 mg, 545.0 μmol). The reaction mixture was stirred for 1 h at 40° C. The mixture was dissolved in EtOAc (50 mL), washed with brine (2×50 mL), dried over anhydrous Na₂SO₄ and concentrated under vacuum. The residue was purified by Prep-TLC with EtOAc/PE (1:4) to afford 8-((tert-butyldimethylsilyl)oxy)-2-chloro-N-(5-chloro-6-(2H-1,2,3-triazol-2-yl)pyridin-3-yl)-9,9-dimethyl-8,9-dihydropyrazolo[1,5-a]pyrido[2,3-e]pyrimidine-6(7H)-carboxamide (65 mg, 40% yield) as a light-yellow solid. ¹H NMR (300 MHz, DMSO-d₆) δ 9.45 (s, 1H), 8.74 (s, 1H), 8.61 (d, J=2.4 Hz, 1H), 8.39 (d, J=2.4 Hz, 1H), 8.16 (s, 2H), 6.92 (s, 1H), 4.17-4.23 (m, 1H), 3.96-4.07 (m, 1H), 3.64-3.76 (m, 1H), 1.68 (s, 3H), 1.52 (s, 3H), 0.74 (s, 9H), 0.16 (s, 3H), 0.06 (s, 3H); LC-MS: m/z 588 [M+H]⁺.

### Step 12: 2-chloro-N-(5-chloro-6-(2H-1,2,3-triazol-2-yl)pyridin-3-yl)-8-hydroxy-9,9-dimethyl-8,9-dihydropyrazolo[1,5-a]pyrido[2,3-e]pyrimidine-6(7H)-carboxamide

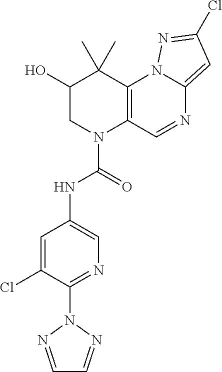 [SMILES: CC1(C)c2c(cnc3cc(Cl)nn23)N(C(=O)Nc2cnc(-n3nccn3)c(Cl)c2)CC1O]

To a solution of 8-((tert-butyldimethylsilyl)oxy)-2-chloro-N-(5-chloro-6-(2H-1,2,3-triazol-2-yl)pyridin-3-yl)-9,9-dimethyl-8,9-dihydropyrazolo[1,5-a]pyrido[2,3-e]pyrimidine-6(7H)-carboxamide (55 mg, 93.5 μmol) in THF (2 mL) was added TBAF (1 M, 2 mL) at 25° C. The resulting mixture was stirred for 4 h at r.t. The reaction mixture was concentrated under reduced pressure. The residue was diluted with water (50 mL) and extracted with EtOAc (3×50 mL). The combined organic layers were washed with saturated aqueous ammonium chloride solution (3×50 mL), dried over anhydrous Na₂SO₄ and concentrated under vacuum. The residue was purified by Prep-TLC with EtOAc to afford 30 mg of the crude product (90% purity). The residue was submitted to Prep-HPLC purification and the collected fractions were lyophilized to give 2-chloro-N-(5-chloro-6-(2H-1,2,3-triazol-2-yl)pyridin-3-yl)-8-hydroxy-9,9-dimethyl-8,9-dihydropyrazolo[1,5-a]pyrido[2,3-e]pyrimidine-6(7H)-carboxamide (19.3 mg, 45% yield) as a white solid. ¹H NMR (300 MHz, DMSO-d₆) δ 9.95 (s, 1H), 8.69 (s, 1H), 8.63 (d, J=2.4 Hz, 1H), 8.37 (d, J=2.4 Hz, 1H), 8.15 (s, 2H), 6.88 (s, 1H), 5.62-5.63 (m, 1H), 4.03-4.09 (m, 1H), 3.72-3.77 (m, 2H), 1.64 (s, 3H), 1.57 (s, 3H); LC-MS: m/z 474 [M+H]⁺.

### Step 13: Separation of enantiomers to obtain (S)-2-chloro-N-(5-chloro-6-(2H-1,2,3-triazol-2-yl)pyridin-3-yl)-8-hydroxy-9,9-dimethyl-8,9-dihydropyrazolo[1,5-a]pyrido[2,3-e]pyrimidine-6(7H)-carboxamide and (R)-2-chloro-N-(5-chloro-6-(2H-1,2,3-triazol-2-yl)pyridin-3-yl)-8-hydroxy-9,9-dimethyl-8,9-dihydropyrazolo[1,5-a]pyrido[2,3-e]pyrimidine-6(7H)-carboxamide

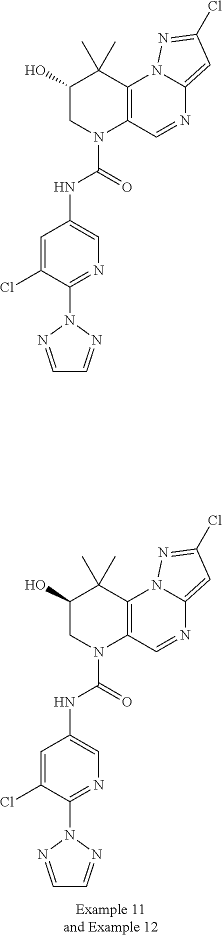 [SMILES: CC1(C)c2c(cnc3cc(Cl)nn23)N(C(=O)Nc2cnc(-n3nccn3)c(Cl)c2)C[C@@H]1O ; CC1(C)c2c(cnc3cc(Cl)nn23)N(C(=O)Nc2cnc(-n3nccn3)c(Cl)c2)C[C@H]1O]

2-chloro-N-(5-chloro-6-(2H-1,2,3-triazol-2-yl)pyridin-3-yl)-8-hydroxy-9,9-dimethyl-8,9-dihydropyrazolo[1,5-a]pyrido[2,3-e]pyrimidine-6(7H)-carboxamide (16 mg, 33.7 μmol) was submitted to chiral HPLC purification (Column: CHIRALPAK IF, 2×25 cm, 5 um; Mobile Phase A: Hex (0.5% 2M NH₃-MeOH)—HPLC, Mobile Phase B: EtOH—HPLC; Flow rate: 14 mL/min; isocratic 45% B; 220/254 nm; RT1: 11.82; RT2: 14.305; Injection Volume: 3.8 ml; Number of Runs: 1). The first eluting isomer was concentrated and lyophilized to afford Example 11 (6.4 mg, 40% yield) as a light-yellow solid. The second eluting isomer was concentrated and lyophilized to afford Example 12 (7.4 mg, 46% yield) as a white solid.

Example 11: ¹H NMR (300 MHz, DMSO-d₆) δ: 9.97 (s, 1H), 8.69 (s, 1H), 8.63 (d, J=2.4 Hz, 1H), 8.38 (d, J=2.4 Hz, 1H), 8.16 (s, 2H), 6.89 (s, 1H), 5.64-5.65 (m, 1H), 4.04-4.11 (m, 1H), 3.72-3.76 (m, 2H), 1.64 (s, 3H), 1.57 (s, 3H); LC-MS: m/z 474 [M+H]⁺.

Example 12: ¹H NMR (300 MHz, DMSO-d₆) δ: 9.97 (s, 1H), 8.69 (s, 1H), 8.63 (d, J=2.4 Hz, 1H), 8.38 (d, J=2.4 Hz, 1H), 8.16 (s, 2H), 6.89 (s, 1H), 5.64-5.65 (m, 1H), 4.04-4.10 (m, 1H), 3.72-3.76 (m, 2H), 1.64 (s, 3H), 1.57 (s, 3H); LC-MS: m/z 474 [M+H]⁺.

### Method J1

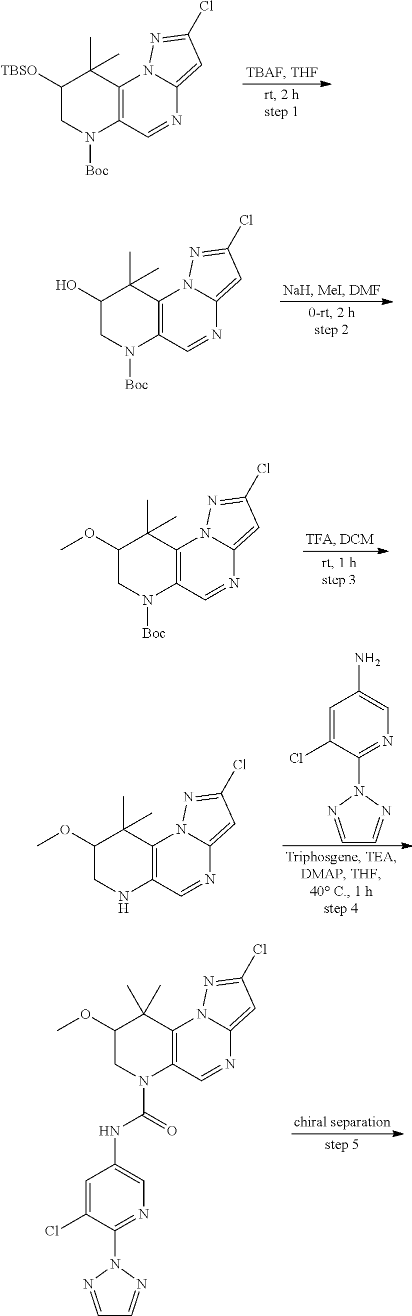 [SMILES: CC(C)(C)OC(=O)N1CC(O[Si](C)(C)C(C)(C)C)C(C)(C)c2c1cnc1cc(Cl)nn21 ; CC(C)(C)OC(=O)N1CC(O)C(C)(C)c2c1cnc1cc(Cl)nn21 ; COC1CN(C(=O)OC(C)(C)C)c2cnc3cc(Cl)nn3c2C1(C)C ; COC1CNc2cnc3cc(Cl)nn3c2C1(C)C ; Nc1cnc(-n2nccn2)c(Cl)c1 ; COC1CN(C(=O)Nc2cnc(-n3nccn3)c(Cl)c2)c2cnc3cc(Cl)nn3c2C1(C)C]

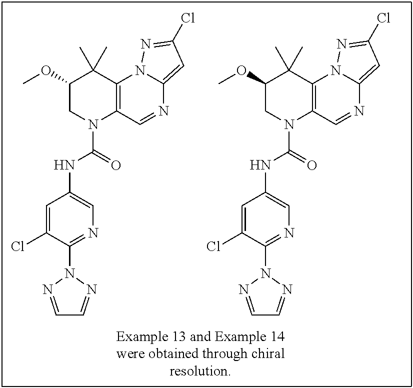 [SMILES: CO[C@H]1CN(C(=O)Nc2cnc(-n3nccn3)c(Cl)c2)c2cnc3cc(Cl)nn3c2C1(C)C ; CO[C@@H]1CN(C(=O)Nc2cnc(-n3nccn3)c(Cl)c2)c2cnc3cc(Cl)nn3c2C1(C)C]

### Examples 13 and 14: Single enantiomers obtained from a racemic mixture containing (R)-2-chloro-N-(5-chloro-6(2H-1,2,3-triazol-2-yl)pyridin-3-yl)-8-methoxy-9,9-dimethyl-8,9-dihydropyrazolo[1,5-a]pyrido[2,3-e]pyrimidine-6(7H)-carboxamide and (S)-2-chloro-N-(5-chloro-6-(2H-1,2,3-triazol-2-yl)pyridin-3-yl)-8-methoxy-9,9-dimethyl-8,9-dihydropyrazolo[1,5-a]pyrido[2,3-e]pyrimidine-6(7H)-carboxamide

### Step 1: tert-butyl 2-chloro-8-hydroxy-9,9-dimethyl-8,9-dihydropyrazolo[1,5-a]pyrido [2,3-e]pyrimidine-6(7H)-carboxylate

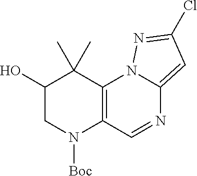 [SMILES: CC(C)(C)OC(=O)N1CC(O)C(C)(C)c2c1cnc1cc(Cl)nn21]

To a mixture of tert-butyl 8-((tert-butyldimethylsilyl)oxy)-2-chloro-9,9-dimethyl-8,9-dihydropyrazolo[1,5-a]pyrido[2,3-e]pyrimidine-6(7H)-carboxylate (250 mg, 536.5 μmol) in THF (5 mL) was added TBAF (1 M in THF, 5 mL) at 25° C. and the mixture was stirred for 2 h at this temperature. The mixture was concentrated under reduced pressure and the residue was purified by Prep-TLC with EtOAc/PE(1:1) to afford tert-butyl 2-chloro-8-hydroxy-9,9-dimethyl-8,9-dihydropyrazolo[1,5-a]pyrido[2,3-e] pyrimidine-6(7H)-carboxylate (120 mg, 63% yield) as a light yellow solid. LC-MS: m/z 353 [M+H]⁺.

### Step 2: tert-butyl 2-chloro-8-methoxy-9,9-dimethyl-8,9-dihydropyrazolo[1,5-a]pyrido[2,3-e]pyrimidine-6(7H)-carboxylate

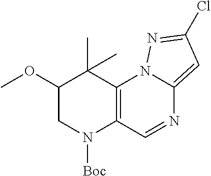 [SMILES: COC1CN(C(=O)OC(C)(C)C)c2cnc3cc(Cl)nn3c2C1(C)C]

To a mixture of tert-butyl 2-chloro-8-hydroxy-9,9-dimethyl-8,9-dihydropyrazolo [1,5-a]pyrido[2,3-e]pyrimidine-6(7H)-carboxylate (120 mg, 340.9 μmol) in DMF (8 mL) was added NaH (60% in mineral oil, 16 mg, 409.1 μmol) at 0° C. The mixture was stirred for 0.5 h at 0° C. MeI (58 mg, 409.1 μmol) was added dropwise and the resulting mixture was stirred for 1 h at 25° C. The mixture was poured into ice/water (50 mL) and extracted with EtOAc (3×50 mL). The combined organic layers were washed with brine (50 mL), dried over anhydrous Na₂SO₄ and concentrated under vacuum. The residue was purified by Prep-TLC with EtOAc/PE(1:2) to afford tert-butyl 2-chloro-8-methoxy-9,9-dimethyl-8,9-dihydropyrazolo[1,5-a]pyrido[2,3-e]pyrimidine-6(7H)-carboxylate (60 mg, 48% yield) as a white solid. ¹H NMR (400 MHz, CDCl₃-d) δ 8.68 (s, 1H), 6.56 (s, 1H), 4.26-4.30 (m, 1H), 3.44-3.48 (m, 4H), 3.17-3.19 (m, 1H), 1.72 (s, 3H), 1.62 (s, 3H), 1.53 (s, 9H); LC-MS: m/z 367 [M+H]⁺.

### Step 3: 2-chloro-8-methoxy-9,9-dimethyl-6,7,8,9-tetrahydropyrazolo[1,5-a]pyrido [2,3-e]pyrimidine

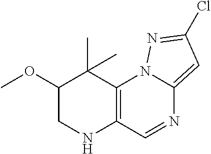 [SMILES: COC1CNc2cnc3cc(Cl)nn3c2C1(C)C]

To a mixture of tert-butyl 2-chloro-8-methoxy-9,9-dimethyl-8,9-dihydropyrazolo [1,5-a]pyrido[2,3-e]pyrimidine-6(7H)-carboxylate (50 mg, 136.3 μmol) in DCM (4 mL) was added TFA (1 mL). The mixture was stirred for 1 h at 25° C. The mixture was concentrated under reduced pressure and the residue was purified by Prep-TLC with EtOAc/PE(1:1) to afford 2-chloro-8-methoxy-9,9-dimethyl-6,7,8,9-tetrahydropyrazolo [1,5-a]pyrido [2,3-e]pyrimidine (35 mg, 96% yield) as a white solid. ¹H NMR (300 MHz, CDC-d) δ: 8.16 (s, 1H), 6.54 (s, 1H), 3.54 (s, 3H), 3.39-3.40 (m, 2H), 3.29-3.32 (m, 1H), 1.73 (s, 3H), 1.69 (s, 3H). LC-MS: m/z 267 [M+H]⁺.

### Step 4: 2-chloro-N-(5-chloro-6-(2H-1,2,3-triazol-2-yl)pyridin-3-yl)-8-methoxy-9,9-dimethyl-8,9-dihydropyrazolo[1,5-a]pyrido[2,3-e]pyrimidine-6(7H)-carboxamide

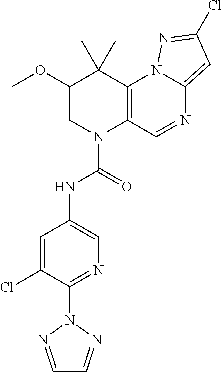 [SMILES: COC1CN(C(=O)Nc2cnc(-n3nccn3)c(Cl)c2)c2cnc3cc(Cl)nn3c2C1(C)C]

To a mixture of 5-chloro-6-(triazol-2-yl)pyridin-3-amine (Method A1 step 2; 26 mg, 133.3 μmol) in THF (4 mL) were added triphosgene (20 mg, 67.5 μmol), TEA (17 mg, 168.7 μmol) at 25° C. The resulting mixture was stirred for 1 h at 25° C. and then filtered. The filtrate was added to a solution of 2-chloro-8-methoxy-9,9-dimethyl-6,7,8,9-tetrahydropyrazolo[1,5-a]pyrido[2,3-e]pyrimidine (30 mg, 112.5 μmol) in THF (4 mL). To this solution was then added TEA (114 mg, 1.1 mmol) and DMAP (27 mg, 224.9 μmol). The resulting mixture was stirred for 1 h at 40° C. The mixture was poured into water (20 mL) and extracted with EtOAc (3×20 mL). The combined organic layers were washed with brine (50 mL), dried over anhydrous Na₂SO₄ and concentrated under vacuum. The residue was purified by Prep-HPLC and the collected fractions were lyophilized to give 2-chloro-N-(5-chloro-6-(2H-1,2,3-triazol-2-yl)pyridine-3-yl)-8-methoxy-9,9-dimethyl-8,9-dihydropyrazolo[1,5-a]pyrido[2,3-e]pyrimidine-6(7H)-carboxamide (9.3 mg, 17% yield) as a white solid. LC-MS: m/z 488 [M+H]⁺.

### Step 5: Separation of enantiomers to obtain (R)-2-chloro-N-(5-chloro-6-(2H-1,2,3-triazol-2-yl)pyridin-3-yl)-8-methoxy-9,9-dimethyl-8,9-dihydropyrazolo[1,5-a]pyrido[2,3-e]pyrimidine-6(7H)-carboxamide and (S)-2-chloro-N-(5-chloro-6-(2H-1,2,3-triazol-2-yl)pyridin-3-yl)-8-methoxy-9,9-dimethyl-8,9-dihydropyrazolo[1,5-a]pyrido[2,3-e]pyrimidine-6(7H)-carboxamide

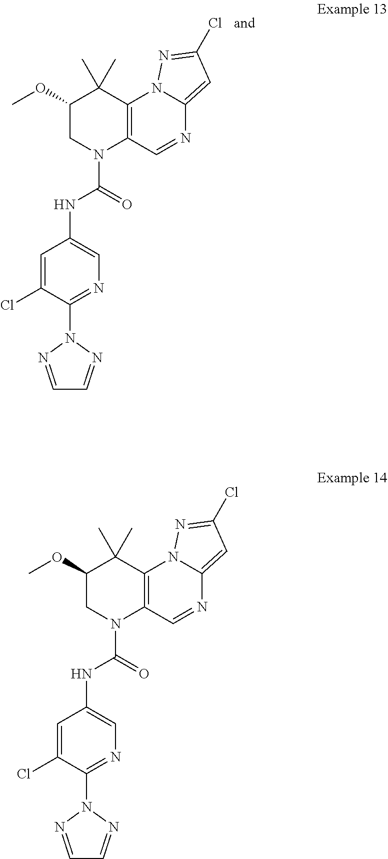 [SMILES: CO[C@H]1CN(C(=O)Nc2cnc(-n3nccn3)c(Cl)c2)c2cnc3cc(Cl)nn3c2C1(C)C ; "and" ; CO[C@@H]1CN(C(=O)Nc2cnc(-n3nccn3)c(Cl)c2)c2cnc3cc(Cl)nn3c2C1(C)C]

2-chloro-N-(5-chloro-6-(2H-1,2,3-triazol-2-yl)pyridin-3-yl)-8-methoxy-9,9-dimethyl-8,9-dihydropyrazolo[1,5-a]pyrido[2,3-e]pyrimidine-6(7H)-carboxamide (7 mg, 14 μmol) was submitted to chiral HPLC purification (Column: CHIRALPAK IA, 2×25 cm, 5 um; Mobile Phase A: Hex (0.5% 2M NH3-MeOH)—HPLC, Mobile Phase B: EtOH—HPLC; Flow rate: 16 mL/min; isocratic 50% B; 220/254 nm; RT1: 9.279; RT2: 13.158; Injection Volume: 1 ml; Number of Runs: 1). The first eluting isomer was concentrated and lyophilized to afford Example 13 (2 mg, 28% yield) as a yellow solid. The second eluting isomer was concentrated and lyophilized to afford Example 14 (2.8 mg, 40% yield) as a yellow solid.

Example 13: ¹H NMR (400 MHz, CDCl₃) δ: 8.53 (s, 1H), 8.43 (d, J=2.0 Hz, 1H), 8.39 (d, J=2.4 Hz, 1H), 7.93 (s, 2H), 7.09 (s, 1H), 6.66 (s, 1H), 4.67-4.71 (m, 1H), 3.43-3.47 (m, 4H), 3.32 (d, J=4.0 Hz, 1H), 1.84 (s, 3H), 1.65 (s, 3H); LC-MS: m/z 488 [M+H]⁺.

Example 14: ¹H NMR (400 MHz, CDCl₃) δ: 8.53 (s, 1H), 8.43 (d, J=2.4 Hz, 1H), 8.39 (d, J=2.4 Hz, 1H), 7.93 (s, 2H), 7.11 (s, 1H), 6.66 (s, 1H), 4.67-4.71 (m, 1H), 3.43-3.47 (m, 4H), 3.32 (d, J=4.0 Hz, 1H), 1.84 (s, 3H), 1.65 (s, 3H); LC-MS: m/z 488 [M+H]⁺.

### Method K1

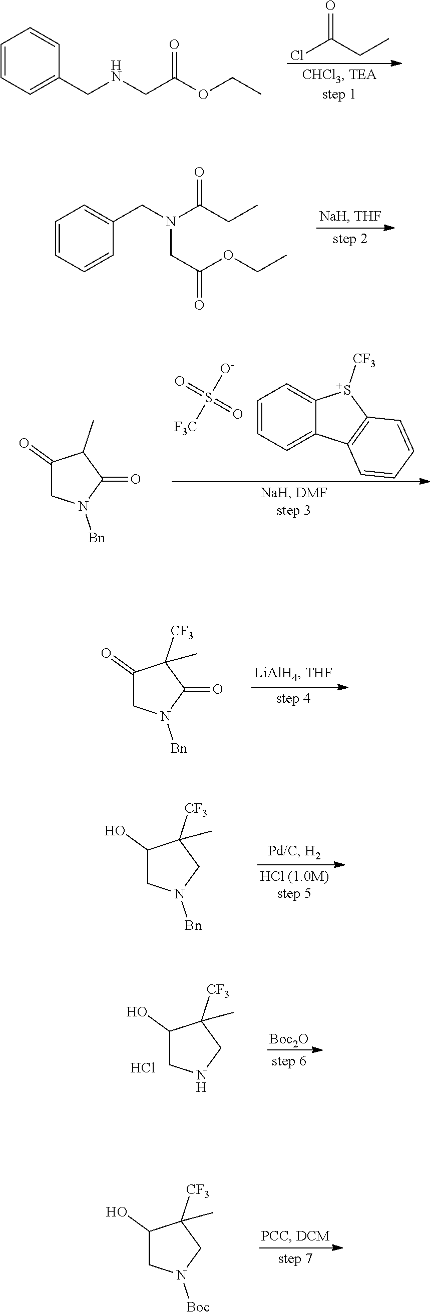

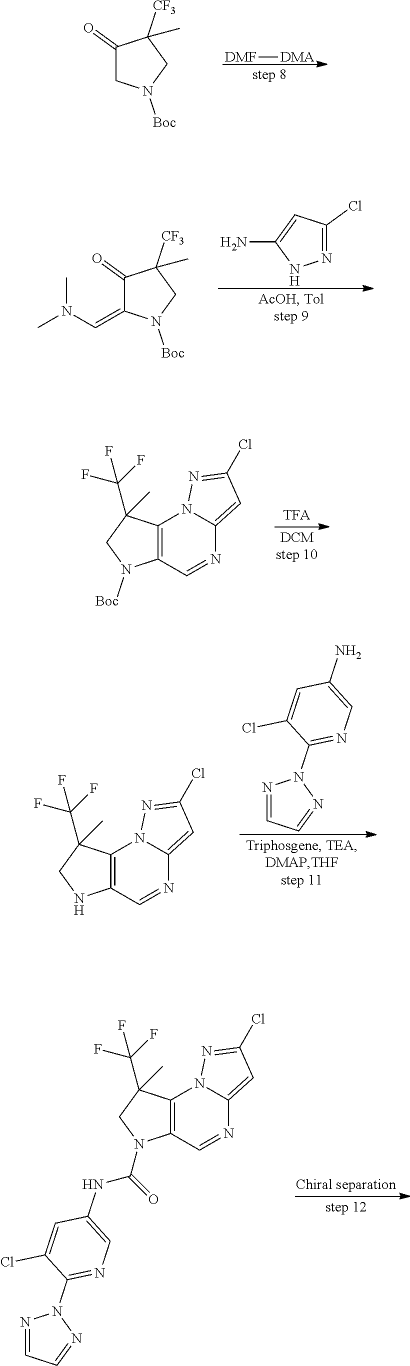

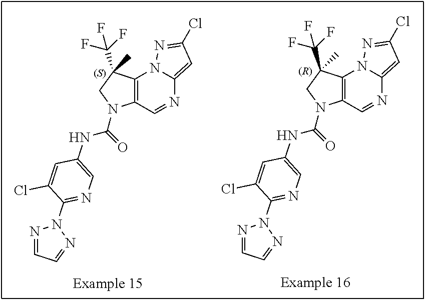

### Examples 15 and 16: (S)-2-chloro-N-(5-chloro-6-(2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(5-chloro-6-(2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: ethyl N-benzyl-N-propionylglycinate

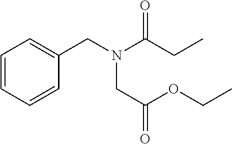 [SMILES: CCOC(=O)CN(Cc1ccccc1)C(=O)CC]

Into a 250 mL 3-necked flask was placed ethyl 2-(benzylamino)acetate (13.5 g, 69.9 mmol), CHCl₃ (130 mL), N,N-diethylethanamine (14.2 g, 139.7 mmol). The propanoyl chloride (7.1 g, 76.9 mmol) in 20 mL CHCl₃ was added dropwise at 0° C. The mixture was stirred for 1 h at 25° C. The mixture was poured into 200 mL of H₂O. The resulting solution was extracted with DCM (3×200 mL). The organic layers were combined, dried and concentrated under vacuum. The residue was applied on a silica gel column and eluted with EtOAc/PE (1:2) to give ethyl N-benzyl-N-propionylglycinate (15.7 g, 81% yield) as a light yellow oil. ¹H NMR (400 MHz, CDCl₃) δ: 7.16-7.37 (m, 5H), 4.61-4.64 (m, 2H), 4.08-4.17 (m, 2H), 3.90-4.03 (m, 2H), 2.28-2.47 (m, 2H), 1.14-1.25 (m, 6H). LC-MS: m/z 250 [M+H]⁺.

### Step 2: 1-benzyl-3-methylpyrrolidine-2,4-dione

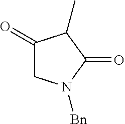

Into a 250 mL 3-necked flask was placed NaH (963 mg, 24.1 mmol) and THF (100 mL). Ethyl N-benzyl-N-propionylglycinate (5.0 g, 20.1 mmol) in THF (50 mL) was added dropwise at 75° C. The mixture was stirred for 12 h at 75° C. The reaction was cooled to 20° C., and then water (20 mL) was added. The mixture was concentrated under vacuum. The residue was applied on a silica gel column and eluted with MeOH/DCM (1:30) to give 1-benzyl-3-methylpyrrolidine-2,4-dione (2.2 g, 49% yield) as an off-white solid. ¹H NMR (400 MHz, DMSO-d₆) δ: 10.62 (s, 1H), 7.14-7.34 (m, 5H), 4.44 (s, 2H), 3.62 (d, J=1.6 Hz, 2H), 1.57 (s, 3H). LC-MS: m/z 204 [M+H]⁺.

### Step 3: 1-benzyl-3-methyl-3-(trifluoromethyl)pyrrolidine-2,4-dione

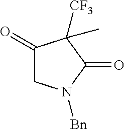 [SMILES: CC1(C(F)(F)F)C(=O)CN(Cc2ccccc2)C1=O]

Into a 100 mL of round bottle flask were placed 1-benzyl-3-methylpyrrolidine-2,4-dione (500 mg, 2.5 mmol) and DMF (10 mL). NaH (94 mg, 2.5 mmol) was added by portions at 0° C. The mixture was stirred for 0.5 h at 25° C. Trifluoromethanesulfonate; 5-(trifluoromethyl)dibenzothiophen-5-ium (989 mg, 2.5 mmol) was added at −55° C. The mixture was stirred for 1 h at −55° C. The reaction mixture was gradually warmed up to 25° C. and stirred for 1 h. The mixture was poured into a mixture of of ice/water (40 mL). The resulting mixture was extracted with EtOAc (3×40 mL). The organic layers were combined, dried over anhydrous Na₂SO₄ and concentrated under vacuum. The residue was applied on a silica gel column and eluted with EtOAc/PE (1:4) to give 1-benzyl-3-methyl-3-(trifluoromethyl)pyrrolidine-2,4-dione (670 mg, 90% yield) as a light yellow oil. ¹H NMR (300 MHz, DMSO-d₆) δ: 7.28-7.42 (m, 5H), 4.84 (d, J=15.03 Hz, 1H), 4.45 (d, J=15.06 Hz, 1H), 4.05 (s, 2H), 1.48 (s, 3H). LC-MS: m/z 272 [M+H]⁺.

### Step 4: 1-benzyl-4-methyl-4-(trifluoromethyl)pyrrolidin-3-ol

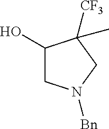 [SMILES: CC1(C(F)(F)F)CN(Cc2ccccc2)CC1O]

Into a 100 mL of round bottle flask was placed 1-benzyl-3-methyl-3-(trifluoromethyl)pyrrolidine-2,4-dione (620 mg, 2.3 mmol) and THF (20 mL). LiAlH₄ (582 mg, 15.3 mmol) was added at 0° C. The reaction mixture was warmed up to 80° C. and stirred at this temperature for 15 h. The reaction mixture was cooled to 0° C. While stirring, 582 mg of H₂O and 582 mg of aqueous NaOH solution (10%) were added, followed by the addition of 582 mg of H₂O. The mixture was stirred for 10 min at 25° C. and the precipitate was filtered off. The filtrate was concentrated under vacuum. The residue was applied on a silica gel column and eluted with MeOH/DCM (1:50) to give 1-benzyl-4-methyl-4-(trifluoromethyl)pyrrolidin-3-ol (530 mg, 80% yield) as a colorless oil. ¹H NMR (400 MHz, DMSO-d₆) δ: 7.20-7.32 (m, 5H), 5.30 (d, J=5.88 Hz, 1H), 3.92-3.97 (m, 1H), 3.50-3.61 (m, 2H), 3.00-3.04 (m, 1H), 2.65 (d, J=9.48 Hz, 1H), 2.50 (s, 1H), 2.24-2.28 (m, 1H), 1.21 (s, 3H). LC-MS: m/z 260 [M+H]⁺.

### Step 5: 4-methyl-4-(trifluoromethyl)pyrrolidin-3-ol hydrochloride

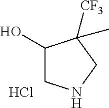

Into a 100 mL of round bottle flask were placed 1-benzyl-4-methyl-4-(trifluoromethyl)pyrrolidin-3-ol (430 mg, 1.7 mmol), EtOH (15 mL), HCl (1.0 M, 1.7 mL) and Pd/C (100 mg, 10%). The flask was evacuated and flushed three times with nitrogen, followed by flushing with hydrogen. The mixture was stirred for 18 h at 25° C. under an atmosphere of hydrogen (balloon). HCl (1.0 M, 1.7 mL) was added with stirring. The mixture was stirred for 15 min at 25° C. The solid was filtered out. The filtrate was concentrated under vacuum. This resulted in 4-methyl-4-(trifluoromethyl)pyrrolidin-3-ol hydrochloride (300 mg, 79% yield) as a yellow solid. LC-MS: m/z 170 [M+H]⁺.

### Step 6: tert-butyl 4-hydroxy-3-methyl-3-(trifluoromethyl)pyrrolidine-1-carboxylate

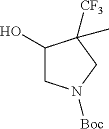 [SMILES: CC(C)(C)OC(=O)N1CC(O)C(C)(C(F)(F)F)C1]

Into a 100 mL of round bottle flask were placed 4-methyl-4-(trifluoromethyl)pyrrolidin-3-ol hydrochloride (300 mg, 1.5 mmol), THF (15.0 mL), (Boc)₂O (477 mg, 2.2 mmol) and N,N-diethylethanamine (738 mg, 7.3 mmol). The mixture was stirred for 2 h at 25° C. The mixture was concentrated under vacuum. The residue was applied on a silica gel column and eluted with EtOAc/PE (1:4) to give tert-butyl 4-hydroxy-3-methyl-3-(trifluoromethyl)pyrrolidine-1-carboxylate (370 mg, 85% yield) as a white solid. ¹H NMR (300 MHz, DMSO-d₆) δ: 5.60-5.62 (m, 1H), 4.04-4.12 (m, 1H), 3.54-3.61 (m, 2H), 3.14-3.21 (m, 2H), 1.40 (s, 9H), 1.18 (s, 3H). LC-MS: m/z 270 [M+H]⁺.

### Step 7: tert-butyl 3-methyl-4-oxo-3-(trifluoromethyl)pyrrolidine-1-carboxylate

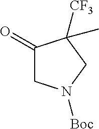 [SMILES: CC(C)(C)OC(=O)N1CC(=O)C(C)(C(F)(F)F)C1]

Into a 100 mL of round bottle flask were placed tert-butyl 4-hydroxy-3-methyl-3-(trifluoromethyl)pyrrolidine-1-carboxylate (300 mg, 1.1 mmol), DCM (15 mL), PCC (1.2 g, 5.6 mmol) and silica gel (600 mg). The mixture was stirred for 12 h at 40° C. The mixture was concentrated under vacuum. The residue was applied on a silica gel column and eluted with EtOAc/PE (1:10) to give tert-butyl 3-methyl-4-oxo-3-(trifluoromethyl)pyrrolidine-1-carboxylate (200 mg, 60% yield) as a white solid. ¹H NMR (400 MHz, DMSO-d₆) δ: 3.92-4.00 (m, 3H), 3.56-3.62 (m, 1H), 1.41 (s, 9H), 1.33 (s, 3H). LC-MS: m/z 268 [M+H]⁺.

### Step 8: tert-butyl (E)-2-((dimethylamino)methylene)-4-methyl-3-oxo-4-(trifluoromethyl)pyrrolidine-1-carboxylate

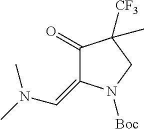

Into a 100 mL of round bottle flask were placed tert-butyl 3-methyl-4-oxo-3-(trifluoromethyl)pyrrolidine-1-carboxylate (160 mg, 598.7 umol) and DMF-DMA (1:1, 6.0 mL). The mixture was stirred for 1 h at 35° C. The mixture was concentrated under vacuum to give tert-butyl (E)-2-((dimethylamino)methylene)-4-methyl-3-oxo-4-(trifluoromethyl)pyrrolidine-1-carboxylate (193 mg, crude) as a light yellow oil. LC-MS: m/z 323 [M+H]⁺.

### Step 9: tert-butyl 2-chloro-8-methyl-8-(trifluoromethyl)-7,8-dihydro-6H-pyrazolo[1,5-a]pyrrolo[2,3-e]pyrimidine-6-carboxylate

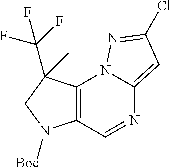 [SMILES: CC(C)(C)OC(=O)N1CC(C)(C(F)(F)F)c2c1cnc1cc(Cl)nn21]

Into a 100 mL of round bottle flask were placed tert-butyl (E)-2-((dimethylamino)methylene)-4-methyl-3-oxo-4-(trifluoromethyl)pyrrolidine-1-carboxylate (193 mg, 598.8 umol), 3-chloro-1H-pyrazol-5-amine (70 mg, 598.8 umol), toluene (10 mL) and HOAc (1.0 mL). The mixture was stirred for 15 h at 95° C. The reaction was cooled to 25° C. and concentrated under vacuum. 20 mL of NaHCO₃ (aq., sat.) was added. The resulting solution was extracted with EtOAc (3×20 mL). The organic layers were combined, dried and concentrated under vacuum. The residue was applied on a silica gel column and eluted with EtOAc/PE (13:87) to give tert-butyl 2-chloro-8-methyl-8-(trifluoromethyl)-7,8-dihydro-6H-pyrazolo[1,5-a]pyrrolo[2,3-e]pyrimidine-6-carboxylate (90 mg, 36% yield) as a yellow oil. ¹H NMR (400 MHz, DMSO-d₆) δ: 9.14 (s, 1H), 7.04 (s, 1H), 3.95-4.03 (m, 2H), 1.52 (s, 9H), 1.47 (s, 3H). LC-MS: m/z 377 [M+H]⁺.

### Step 10: 2-chloro-8-methyl-8-(trifluoromethyl)-7,8-dihydro-6H-pyrazolo[1,5-a]pyrrolo[2,3-e]pyrimidine

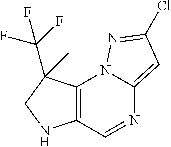 [SMILES: CC1(C(F)(F)F)CNc2cnc3cc(Cl)nn3c21]

Into a 40 mL vial were placed tert-butyl 2-chloro-8-methyl-8-(trifluoromethyl)-7,8-dihydro-6H-pyrazolo[1,5-a]pyrrolo[2,3-e]pyrimidine-6-carboxylate (80 mg, 212.3 umol), DCM (6 mL) and TFA (2 mL). The mixture was stirred for 1 h at 25° C. The mixture was concentrated under vacuum. 20 mL of NaHCO₃ (aq., sat.) was added. The resulting mixture was extracted with DCM (3×20 mL). The organic layers were combined, dried and concentrated under vacuum. The residue was purified by prep-TLC eluting with MeOH/DCM (1:35). This resulted in 2-chloro-8-methyl-8-(trifluoromethyl)-7,8-dihydro-6H-pyrazolo[1,5-a]pyrrolo[2,3-e]pyrimidine (32 mg, 49% yield) as a yellow oil. ¹H NMR (300 MHz, DMSO-d₆) δ: 8.37 (s, 1H), 6.82 (s, 1H), 5.95-5.99 (br, 1H), 3.87-3.92 (m, 1H), 3.54-3.59 (m, 1H), 1.79 (s, 3H). LC-MS: m/z 277 [M+H]⁺.

### Step 11: 2-chloro-N-(5-chloro-6-(2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

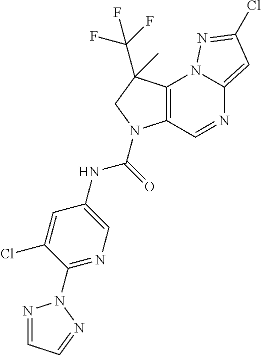 [SMILES: CC1(C(F)(F)F)CN(C(=O)Nc2cnc(-n3nccn3)c(Cl)c2)c2cnc3cc(Cl)nn3c21]

To a stirred mixture of 5-chloro-6-(triazol-2-yl)pyridin-3-amine (Method A1 step 2; 19 mg, 95.4 umol) and bis(trichloromethyl) carbonate (14 mg, 47.7 umol) in THF (5 mL) was added TEA (12 mg, 119.3 umol) dropwise at 0° C. The resulting mixture was stirred for 1 h at 25° C. and then filtered. The resulting filtrate was added to a solution of 2-chloro-8-methyl-8-(trifluoromethyl)-7,8-dihydro-6H-pyrazolo[1,5-a]pyrrolo[2,3-e]pyrimidine (22 mg, 79.5 umol) in THF (1 mL). To this solution was then added TEA (81 mg, 795.2 umol) and N,N-dimethylpyridin-4-amine (19 mg, 159.1 umol). The mixture was stirred for 2 h at 40° C. The reaction mixture was concentrated under vacuum. The residue was purified by Prep-TLC eluting with MeOH/DCM (3.5:120). The crude product (45 mg) was purified by Prep-HPLC. The fractions containing the product were lyophilized to give 2-chloro-N-(5-chloro-6-(2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide (25.1 mg, 61% yield) as a white solid. LC-MS: m/z 498 [M+H]⁺.

### Step 12: Separation of enantiomers to obtain (S)-2-chloro-N-(5-chloro-6-(2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(5-chloro-6-(2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

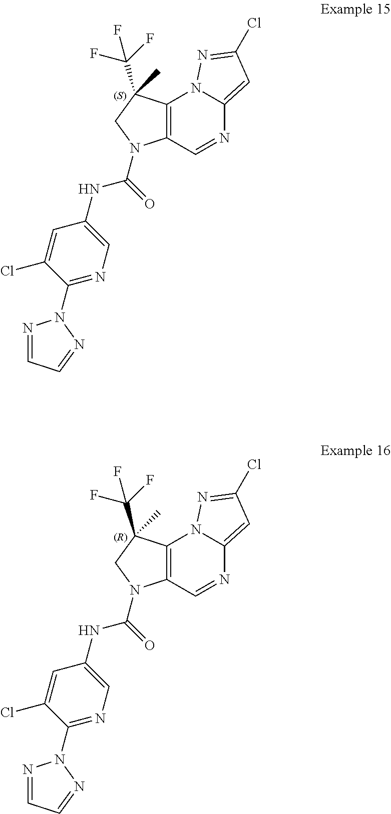

25 mg of 2-chloro-N-(5-chloro-6-(2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide was submitted to chiral HPLC purification (Column: CHIRALPAK IE, 2×25 cm, 5 um; Mobile Phase A:Hex(8 mmol/L NH3·MeOH)—HPLC, Mobile Phase B:EtOH—HPLC; Flow rate: 20 mL/min; isocratic 25% B; 220/254 nm; RT1:8.945; RT2:10.506; Injection Volume: 0.5 ml; Number of Runs: 6). The first eluting isomer was concentrated and lyophilized to afford Example 15 (7.8 mg, 31% yield). The second eluting isomer was concentrated and lyophilized to Example 16 as a white solid (6.1 mg, 24% yield).

Example 15: ¹H NMR (400 MHz, DMSO-d₆) δ: 9.72 (s, 1H), 9.36 (s, 1H), 8.75 (d, J=2.4 Hz, 1H), 8.51 (d, J=2.4 Hz, 1H), 8.18 (s, 2H), 7.09 (s, 1H), 4.86 (d, J=11.6 Hz, 1H), 4.31 (d, J=11.6 Hz, 1H), 1.99 (s, 3H). LC-MS: m/z 498 [M+H]⁺.

Example 16: ¹H NMR (400 MHz, DMSO-d₆) δ: 9.72 (s, 1H), 9.36 (s, 1H), 8.76 (d, J=2.4 Hz, 1H), 8.51 (d, J=2.4 Hz, 1H), 8.18 (s, 2H), 7.09 (s, 1H), 4.86 (d, J=11.6 Hz, 1H), 4.31 (d, J=11.2 Hz, 1H), 1.99 (s, 3H). LC-MS: m/z 498 [M+H]⁺.

### Method L1

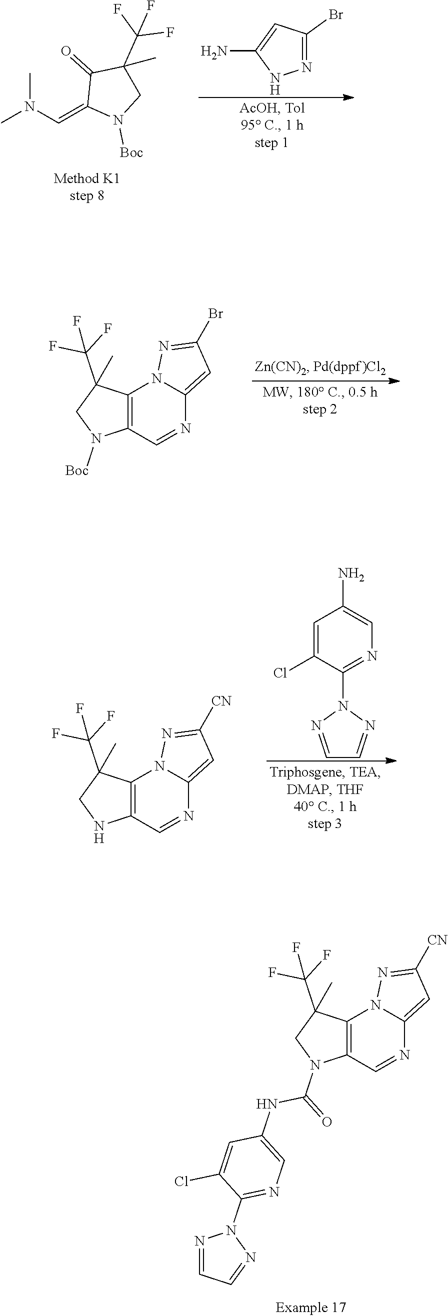 [SMILES: CN(C)/C=C1\C(=O)C(C)(C(F)(F)F)CN1C(=O)OC(C)(C)C ; Nc1cc(Br)n[nH]1 ; CC(C)(C)OC(=O)N1CC(C)(C(F)(F)F)c2c1cnc1cc(Br)nn21 ; CC1(C(F)(F)F)CNc2cnc3cc(C#N)nn3c21 ; Nc1cnc(-n2nccn2)c(Cl)c1 ; CC1(C(F)(F)F)CN(C(=O)Nc2cnc(-n3nccn3)c(Cl)c2)c2cnc3cc(C#N)nn3c21]

### Example 17: N-[5-chloro-6-(triazol-2-yl)-3-pyridyl]-11-cyano-3-methyl-3-(trifluoromethyl)-1,5,8,12-tetrazatricyclo[7.3.0.02,6]dodeca-2(6),7,9,11-tetraene-5-carboxamide

### Step 1: tert-butyl 2-bromo-8-methyl-8-(trifluoromethyl)-7,8-dihydro-6H-pyrazolo [1,5-a]pyrrolo[2,3-e]pyrimidine-6-carboxylate

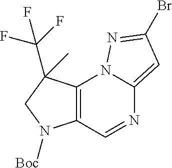 [SMILES: CC(C)(C)OC(=O)N1CC(C)(C(F)(F)F)c2c1cnc1cc(Br)nn21]

To a mixture of tert-butyl (E)-2-((dimethylamino)methylene)-4-methyl-3-oxo-4-(trifluoromethyl)pyrrolidine-1-carboxylate (Method K1 step 8; 500 mg, 1.6 mmol) in toluene (10 mL) were added AcOH (1 mL) and 3-bromo-1H-pyrazol-5-amine (304 mg, 1.9 mmol) at 25° C. The reaction mixture was stirred for 1 h at 95° C. After cooled to 25° C., the mixture was concentrated under vacuum. The residue was dissolved in EtOAc (100 mL). The mixture was washed with saturated aqueous NaHCO₃ (50 mL×3), dried over anhydrous Na₂SO₄ and concentrated under reduced pressure. The residue was applied onto a silica gel column and eluted with EtOAc/PE (1:4) to afford tert-butyl 2-bromo-8-methyl-8-(trifluoromethyl)-7,8-dihydro-6H-pyrazolo[1,5-a]pyrrolo[2,3-e]pyrimidine-6-carboxylate (188 mg, 29% yield) as a yellow oil. LC-MS: m/z 421 [M+H]⁺.

### Step 2: 8-methyl-8-(trifluoromethyl)-7,8-dihydro-6H-pyrazolo[1,5-a]pyrrolo[2,3-e]pyrimidine-2-carbonitrile

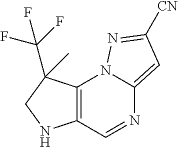 [SMILES: CC1(C(F)(F)F)CNc2cnc3cc(C#N)nn3c21]

To a mixture of tert-butyl 2-bromo-8-methyl-8-(trifluoromethyl)-7,8-dihydro-6H-pyrazolo[1,5-a]pyrrolo[2,3-e]pyrimidine-6-carboxylate (150 mg, 356.1 μmol) in DMF (3 mL) were added Zn(CN)₂ (84 mg, 712.0 μmol) and Pd(dppf)Cl₂ (43.62 mg, 53.4 μmol) under N₂. The reaction mixture was heated in a microwave reactor for 30 min at 180° C. After cooled to 25° C., the mixture was poured into water (50 mL) and extracted with EtOAc (3×50 mL). The combined organic layers were dried over anhydrous Na₂SO₄ and concentrated under vacuum. The residue was purified by Prep-TLC with EtOAc to afford 8-methyl-8-(trifluoromethyl)-7,8-dihydro-6H-pyrazolo[1,5-a]pyrrolo[2,3-e]pyrimidine-2-carbonitrile (50 mg, 38% yield) as a yellow solid. LC-MS: m/z 268 [M+H]⁺.

### Step 3: N-(5-chloro-6-(2H-1,2,3-triazol-2-yl)pyridin-3-yl)-2-cyano-8-methyl-8-(trifluoromethyl)-7,8-dihydro-6H-pyrazolo[1,5-a]pyrrolo[2,3-e]pyrimidine-6-carboxamide

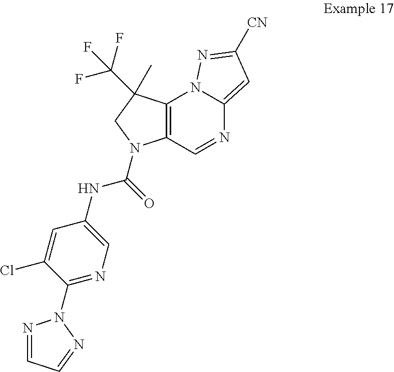 [SMILES: CC1(C(F)(F)F)CN(C(=O)Nc2cnc(-n3nccn3)c(Cl)c2)c2cnc3cc(C#N)nn3c21]

To a mixture of 5-chloro-6-(triazol-2-yl)pyridin-3-amine (Method A1 step 2; 45 mg, 224.5 μmol) in THF (3 mL) were added Triphosgene (34 mg, 112.0 μmol) and TEA (29 mg, 280.5 μmol) at 25° C. The resulting mixture was stirred for 0.5 h at 25° C. and then filtered. The resulting filtrate was added to a solution of 8-methyl-8-(trifluoromethyl)-7,8-dihydro-6H-pyrazolo[1,5-a]pyrrolo[2,3-e]pyrimidine-2-carbonitrile (50 mg, 187.1 μmol) in THF (3 mL). To this solution was then added TEA (190 mg, 1.9 mmol) and N,N-dimethylpyridin-4-amine (46 mg, 374.5 μmol). The resulting mixture was stirred for 1 h at 40° C. The mixture was poured into water (40 mL) and extracted with EtOAc (3×40 mL). The combined organic layers were washed with brine (50 mL), dried over anhydrous Na₂SO₄ and concentrated under vacuum. The residue was submitted to Prep-HPLC purification and fractions containing the product were lyophilized to give N-(5-chloro-6-(2H-1,2,3-triazol-2-yl)pyridine-3-yl)-2-cyano-8-methyl-8-(trifluoromethyl)-7,8-dihydro-6H-pyrazolo[1,5-a]pyrrolo[2,3-e]pyrimidine-6-carboxamide (3.5 mg, 4% yield) as a white solid.

Example 17: ¹H NMR (400 MHz, CDCl₃-d) δ 9.61 (s, 1H), 8.56 (d, J=2.4 Hz, 1H), 8.46 (d, J=2.0 Hz, 1H), 7.98 (s, 2H), 7.00 (d, J=2.8 Hz, 1H), 4.68 (d, J=10.8 Hz, 1H), 4.14 (d, J=10.8 Hz, 1H), 2.11 (s, 3H). LC-MS: m/z 489 [M+H]⁺.

### Method M1

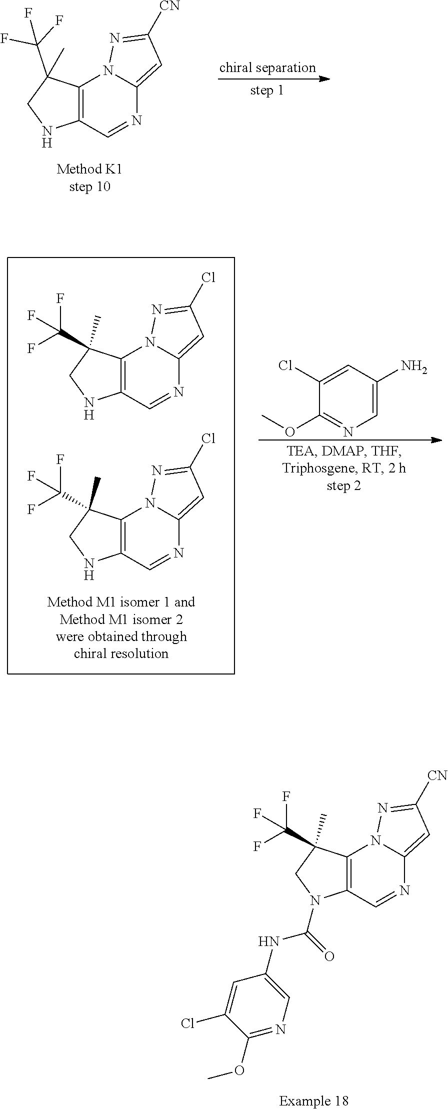

### Example 18: (R)-2-chloro-N-(5-chloro-6-methoxypyridin-3-yl)-8-methyl-8-(trifluoromethyl)-7,8-dihydro-6H-pyrazolo[1,5-a]pyrrolo[2,3-e]pyrimidine-6-carboxamide

### Step 1: (S)-2-chloro-8-methyl-8-(trifluoromethyl)-7,8-dihydro-6H-pyrazolo[1,5-a]pyrrolo[2,3-e]pyrimidine and (R)-2-chloro-8-methyl-8-(trifluoromethyl)-7,8-dihydro-6H-pyrazolo[1,5-a]pyrrolo[2,3-e]pyrimidine

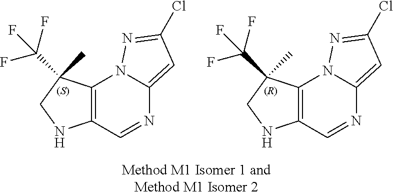

A racemic mixture of 2-chloro-8-methyl-8-(trifluoromethyl)-7,8-dihydro-6H-pyrazolo[1,5-a]pyrrolo[2,3-e]pyrimidine (Method K1 step 10; 2.2 g) was purified by Prep-SFC (Column: CHIRAL ART Amylose-C NEO, 3×25 cm, 5 um; Mobile Phase A: CO₂, Mobile Phase B: MeOH (0.1% 2M NH₃-MeOH); Flow rate: 100 mL/min; isocratic 20% B; 220 nm; RT1: 2.78; RT2: 3.43; Injection Volume: 1 ml; Number of Runs: 30). The first eluting isomer (RT 2.78 min) was concentrated and lyophilized to afford Method M1 isomer 1 (800 mg, 36% yield) as a yellow solid. LC-MS: m/z 277 [M+H]⁺. ee %=99.3%. The second eluting isomer (RT 3.43 min) was concentrated and lyophilized to afford Method M1 isomer 2 as a yellow solid. LC-MS: m/z 277 [M+H]⁺. ee %=98.3%. Both isomers were then individually subjected to Method K1 step 11 for conversion to Example 15 and Example 16, respectively. Example 16 was co-crytallized with the MALT1 enzyme. X-ray crystallography of this complex determined the stereochemistry of Example 16 to be “R”. Example 16 was derived from Method M1 isomer 2.

### Step 2: (R)-2-chloro-N-(5-chloro-6-methoxypyridin-3-yl)-8-methyl-8-(trifluoromethyl)-7,8-dihydro-6H-pyrazolo[1,5-a]pyrrolo[2,3-e]pyrimidine-6-carboxamide (Example 18)

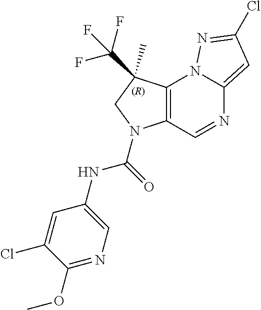

To a stirred solution of 5-chloro-6-methoxy-pyridin-3-amine (14 mg, 86.8 μmol) and bis(trichloromethyl) carbonate (13 mg, 43.4 μmol) in THF (4 mL) was added TEA (11 mg, 108.4 μmol, 15.1 L) at 0° C. The resulting mixture was stirred for 0.5 h at 25° C. and then filtered. The resulting filtrate was added to a solution of Method M1 isomer 2 (20 mg, 72.3 μmol) in THF (1 mL). To this solution was then added TEA (73 mg, 722.9 μmol, 100.8 μL) and N,N-dimethylpyridin-4-amine (18 mg, 144.6 μmol). The mixture was stirred at 25° C. for 2 h. The resulting mixture was purified by Prep-HPLC purification and the collected fractions were lyophilized to give (R)-2-chloro-N-(5-chloro-6-methoxypyridin-3-yl)-8-methyl-8-(trifluoromethyl)-7,8-dihydro-6H-pyrazolo[1,5-a]pyrrolo[2,3-e]pyrimidine-6-carboxamide (15.9 mg, 47% yield) as a white solid. The enantiomer of Example 18 can be prepared analogously using Method M1 isomer 1.

Example 18: ¹H NMR (300 MHz, DMSO-d₆) δ: 9.33 (s, 1H), 9.19 (s, 1H), 8.27 (d, J=2.4 Hz, 1H), 8.13 (d, J=2.4 Hz, 1H), 7.06 (s, 1H), 4.76 (d, J=11.2 Hz, 1H), 4.22 (d, J=11.1 Hz, 1H), 3.93 (s, 3H), 1.97 (s, 3H). LC-MS: m/z 461 [M+H]⁺.

### Example 19: (R)-2-chloro-N-(3-chloro-4-methoxyphenyl)-8-methyl-8-(trifluoromethyl)-7,8-dihydro-6H-pyrazolo[1,5-a]pyrrolo[2,3-e]pyrimidine-6-carboxamide

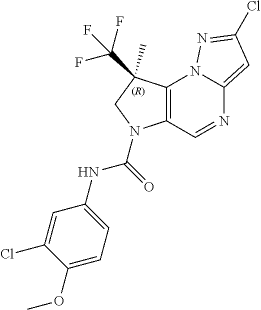

The title compound was prepared according to Method M1 Step 2 by using 3-chloro-4-methoxyaniline and Method M1 isomer 2. The enantiomer of Example 19 can be prepared analogously using Method M1 isomer 1.

Example 19: ¹H NMR (300 MHz, DMSO-d₆) δ: 9.33 (s, 1H), 9.01 (s, 1H), 7.71 (d, J=2.7 Hz, 1H), 7.45-7.49 (m, 1H), 7.15 (d, J=9.0 Hz, 1H), 7.04 (s, 1H), 4.79 (d, J=11.7 Hz, 1H), 4.21 (d, J=11.7 Hz, 1H), 3.84 (s, 3H), 1.97 (s, 3H). LC-MS: m/z 460 [M+H]⁺.

### Method N1

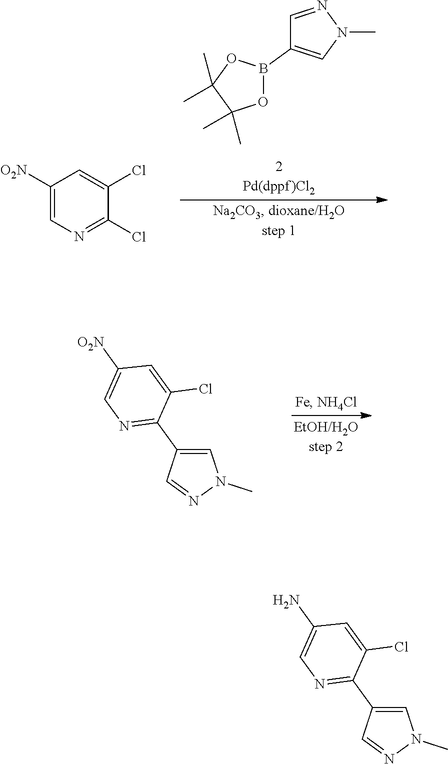

### Step 1: 3-chloro-2-(1-methyl-1H-pyrazol-4-yl)-5-nitropyridine

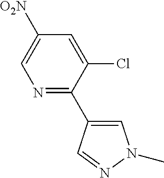 [SMILES: Cn1cc(-c2ncc([N+](=O)[O-])cc2Cl)cn1]

To a stirred mixture of 2,3-dichloro-5-nitro-pyridine (2.00 g, 10.4 mmol) in dioxane (40 mL) and H₂O (20 mL) were added 1-methyl-4-(4,4,5,5-tetramethyl-1,3,2-dioxaborolan-2-yl)-1H-pyrazole (2.37 g, 11.4 mmol), Pd(dppf)Cl₂ (758 mg, 1.0 mmol) and disodium carbonate (2.75 g, 25.9 mmol). The resulting mixture was stirred for 15 h at 100° C. under nitrogen atmosphere. The resulting mixture was cooled down to room temperature, poured into water (100 mL), and was extracted with EtOAc (3×100 mL). The combined organic layers were washed with brine (250 mL), dried over anhydrous Na₂SO₄, filtered and concentrated under reduced pressure. The residue was purified by silica gel column and eluted with EtOAc/PE (3:7) to afford 3-chloro-2-(1-methyl-1H-pyrazol-4-yl)-5-nitropyridine (2.0 g, 83% yield) as an off-white solid. ¹H NMR (300 MHz, Chloroform-d) δ 9.27 (d, J=2.4 Hz, 1H), 8.50 (d, J=2.4 Hz, 1H), 8.38 (s, 1H), 8.32 (s, 1H), 4.01 (s, 3H). LC-MS: m/z 239 [M+H]⁺.

### Step 2: 5-chloro-6-(1-methyl-1H-pyrazol-4-yl)pyridin-3-amine

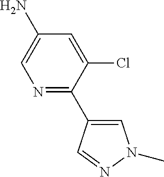

To a stirred mixture of 3-chloro-2-(1-methyl-1H-pyrazol-4-yl)-5-nitropyridine (800 mg, 3.4 mmol) in EtOH (15 mL) and H₂O (15 mL) were added iron (786 mg, 14.1 mmol) and ammonium chloride (753 mg, 14.1 mmol). The resulting mixture was stirred for 1 h at 95° C. The mixture was cooled down to room temperature, filtered and concentrated under reduced pressure to remove EtOH. The aqueous layer was extracted with EtOAc (3×20 mL). The combined organic layers were dried over anhydrous Na₂SO₄, filtered and concentrated under reduced pressure. The residue was purified by silica gel column and eluted with DCM/MeOH (93:7) to afford 5-chloro-6-(1-methyl-1H-pyrazol-4-yl)pyridin-3-amine (510 mg, 73% yield) as a yellow solid. ¹H NMR (300 MHz, DMSO-d₆) δ 8.14 (s, 1H), 7.91 (d, J=2.4 Hz, 1H), 7.87 (s, 1H), 7.02 (d, J=2.4 Hz, 1H), 5.58 (s, 2H), 3.86 (s, 3H). LC-MS: m/z 209 [M+H]⁺.

### Example 20: (R)-2-chloro-N-(5-chloro-6-(1-methyl-1H-pyrazol-4-yl)pyridin-3-yl)-8-methyl-8-(trifluoromethyl)-7,8-dihydro-6H-pyrazolo[1,5-a]pyrrolo[2,3-e]pyrimidine-6-carboxamide

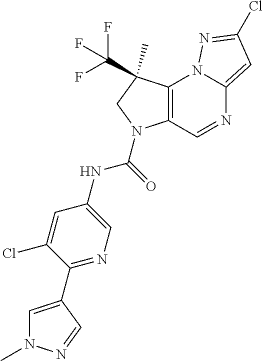 [SMILES: Cn1cc(-c2ncc(NC(=O)N3C[C@@](C)(C(F)(F)F)c4c3cnc3cc(Cl)nn43)cc2Cl)cn1]

The title compound was prepared according to Method M1 step 2 by using 5-chloro-6-(1-methyl-1H-pyrazol-4-yl)pyridin-3-amine and Method M1 isomer 2. The enantiomer of Example 20 can be prepared analogously using Method M1 isomer 1.

Example 20: ¹H NMR (300 MHz, DMSO-d₆) δ 9.40 (s, 1H), 9.35 (s, 1H), 8.69 (d, J=2.1 Hz, 1H), 8.40 (s, 1H), 8.22 (d, J=2.1 Hz, 1H), 8.06 (s, 1H), 7.07 (s, 1H), 4.82 (d, J=11.7 Hz, 1H), 4.27 (d, J=11.7 Hz, 1H), 3.92 (s, 3H), 1.98 (s, 3H). LC-MS: m/z 511 [M+H]⁺.

### Method O1

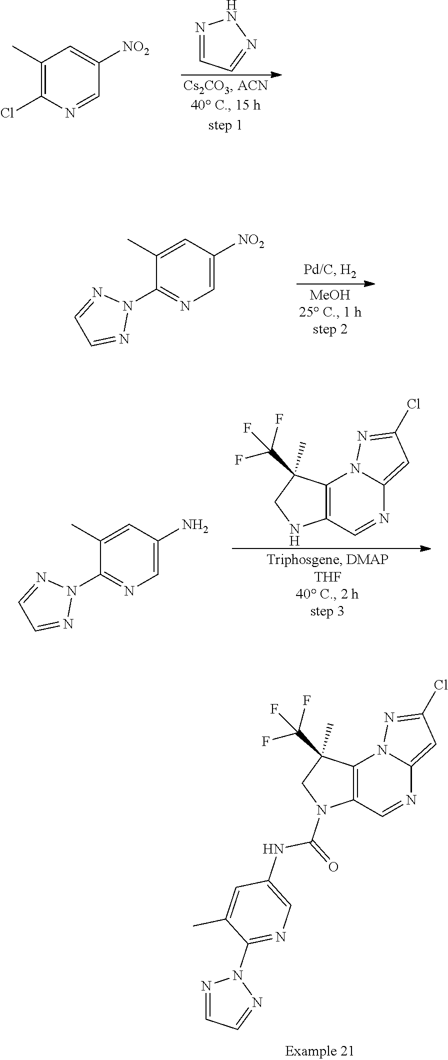 [SMILES: Cc1cc([N+](=O)[O-])cnc1Cl ; c1cn[nH]n1 ; Cc1cc([N+](=O)[O-])cnc1-n1nccn1 ; Cc1cc(N)cnc1-n1nccn1 ; C[C@@]1(C(F)(F)F)CNc2cnc3cc(Cl)nn3c21 ; Cc1cc(NC(=O)N2C[C@@](C)(C(F)(F)F)c3c2cnc2cc(Cl)nn32)cnc1-n1nccn1]

### Example 21: (R)-2-chloro-8-methyl-N-(5-methyl-6-(2H-1,2,3-triazol-2-yl)pyridin-3-yl)-8-(trifluoromethyl)-7,8-dihydro-6H-pyrazolo[1,5-a]pyrrolo[2,3-e]pyrimidine-6-carboxamide

### Step 1: 3-methyl-5-nitro-2-(2H-1,2,3-triazol-2-yl)pyridine

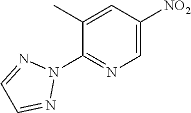

To a stirred solution of 2-chloro-3-methyl-5-nitropyridine (2 g, 11.6 mmol) in ACN (30 mL) were added 2H-1,2,3-triazole (880 mg, 12.7 mmol) and Cs₂CO₃ (2.1 g, 15.1 mmol). The reaction mixture was stirred at 40° C. for 15 h. LCMS showed the reaction was complete. The reaction mixture was concentrated under vacuum. The residue was diluted with water (50 mL) and extracted with EtOAc (2×50 mL). The combined organic layers were concentrated under vacuum. The residue was applied on a silica gel column chromatography and eluted with EtOAc/PE (1:10) to give 3-methyl-5-nitro-2-(2H-1,2,3-triazol-2-yl)pyridine (300 mg, 12% yield) as a white solid. ¹H NMR (300 MHz, DMSO-d₆) δ: 9.25-9.27 (m, 1H), 8.85-8.86 (m, 1H), 8.28 (s, 2H), 2.52-2.53 (m, 3H). LC-MS: m/z 206 [M+H]⁺.

### Step 2: 5-methyl-6-(2H-1,2,3-triazol-2-yl)pyridin-3-amine

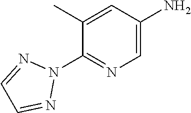

To a stirred solution of 3-methyl-5-nitro-2-(2H-1,2,3-triazol-2-yl)pyridine (50 mg, 243.7 μmol) in MeOH (5 mL) was added Pd/C (25 mg, 10%). The reaction was stirred at 25° C. under hydrogen atmosphere for 1 h. LCMS showed the reaction was completed. The reaction mixture was filtered. The filtrate was concentrated under vacuum. This resulted in 5-methyl-6-(2H-1,2,3-triazol-2-yl)pyridin-3-amine (40 mg, 89% yield) as a colorless oil. ¹H NMR (300 MHz, DMSO-d₆) δ: 8.00-8.03 (s, 2H), 7.70 (d, J=2.7 Hz, 1H), 6.95 (d, J=2.7 Hz, 1H), 5.76 (s, 2H), 1.95 (s, 3H). LC-MS: m/z 176 [M+H]⁺.

### Step 3: (R)-2-chloro-8-methyl-N-(5-methyl-6-(2H-1,2,3-triazol-2-yl)pyridin-3-yl)-8-(trifluoromethyl)-7,8-dihydro-6H-pyrazolo[1,5-a]pyrrolo[2,3-e]pyrimidine-6-carboxamide

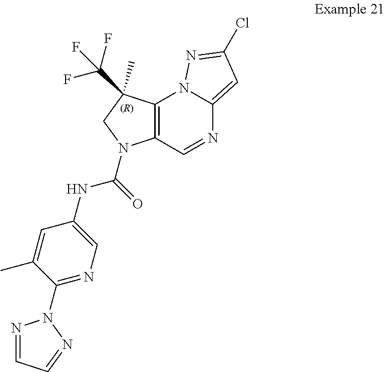

To a stirred solution of 5-methyl-6-(2H-1,2,3-triazol-2-yl)pyridin-3-amine (25 mg, 140.9 μmol) in THF (5 mL) was added triphosgene (19 mg, 65.1 μmol) and TEA (16 mg, 162.7 μmol). The resulting mixture was stirred for 0.5 h at 28° C. and then filtered. The resulting filtrate was added to a solution of Method M1 isomer 2 (30 mg, 108.4 μmol) in THF (1 mL). To this solution was added N,N-dimethylpyridin-4-amine (26 mg, 216.9 μmol) and TEA (110 mg, 1.1 mmol). The mixture was stirred at 40° C. for 2 h. LCMS showed the reaction was complete. The solvent was concentrated under vacuum. The residue was purified by Prep-TLC with MeOH/DCM (1:30) to afford 42 mg of crude product. The crude product was purified by Prep-HPLC to afford (R)-2-chloro-8-methyl-N-(5-methyl-6-(2H-1,2,3-triazol-2-yl)pyridin-3-yl)-8-(trifluoromethyl)-7,8-dihydro-6H-pyrazolo[1,5-a]pyrrolo[2,3-e]pyrimidine-6-carboxamide (25.3 mg, 48% yield) as a white solid. The enantiomer of Example 21 can be prepared analogously using Method M1 isomer 1.

Example 21: ¹H NMR (400 MHz, DMSO-d₆) δ: 9.47 (s, 1H), 9.36 (s, 1H), 8.61 (s, 1H), 8.18 (s, 1H), 8.12 (s, 2H), 7.07 (s, 1H), 4.86 (d, J=11.2 Hz, 1H), 4.29 (d, J=11.2 Hz, 1H), 2.21 (s, 3H), 1.99 (s, 3H). LC-MS: m/z 478 [M+H]⁺

### Example 22: (R)-2-chloro-N-(5-chloro-6-(4-cyano-1H-pyrazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide

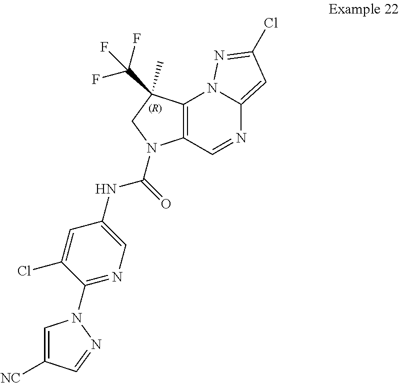

The title compound was prepared according to Method O1 step 3 by using 1-(5-amino-3-chloropyridin-2-yl)-1H-pyrazole-4-carbonitrile and Method M1 isomer 2. The enantiomer of Example 22 can be prepared analogously using Method M1 isomer 1.

Example 22: ¹H NMR (300 MHz, DMSO-d₆) δ: 9.68 (s, 1H), 9.35 (s, 1H), 9.12 (s, 1H), 8.72 (d, J=2.4 Hz, 1H), 8.47 (d, J=2.1 Hz, 1H), 8.40 (s, 1H), 7.08 (s, 1H), 4.84 (d, J=11.7 Hz, 1H), 4.29 (d, J=11.7 Hz, 1H), 1.99 (s, 3H). LC-MS: m/z 522 [M+H]⁺.

### Example 23: (R)-2-chloro-N-(5-chloro-6-(pyrrolidin-1-yl)pyridin-3-yl)-8-methyl-8-(trifluoromethyl)-7,8-dihydro-6H-pyrazolo[1,5-a]pyrrolo[2,3-e]pyrimidine-6-carboxamide

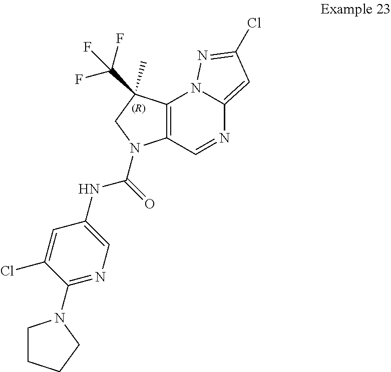

The title compound was prepared according to Method O1 Step 3 by using of 5-chloro-6-(pyrrolidin-1-yl)pyridin-3-amine and Method M1 isomer 2. The enantiomer of Example 23 can be prepared analogously using Method M1 isomer 1.

Example 23: ¹H NMR (300 MHz, DMSO-d₆) δ: 9.32 (s, 1H), 9.01 (s, 1H), 8.19 (d, J=2.1 Hz, 1H), 7.88 (d, J=2.1 Hz, 1H), 7.04 (s, 1H), 4.74 (d, J=11.4 Hz, 1H), 4.21 (d, J=11.4 Hz, 1H), 3.50-3.62 (m, 4H), 1.96 (s, 3H), 1.81-1.91 (m, 4H). LC-MS: m/z 500 [M+H]⁺

### Method P1

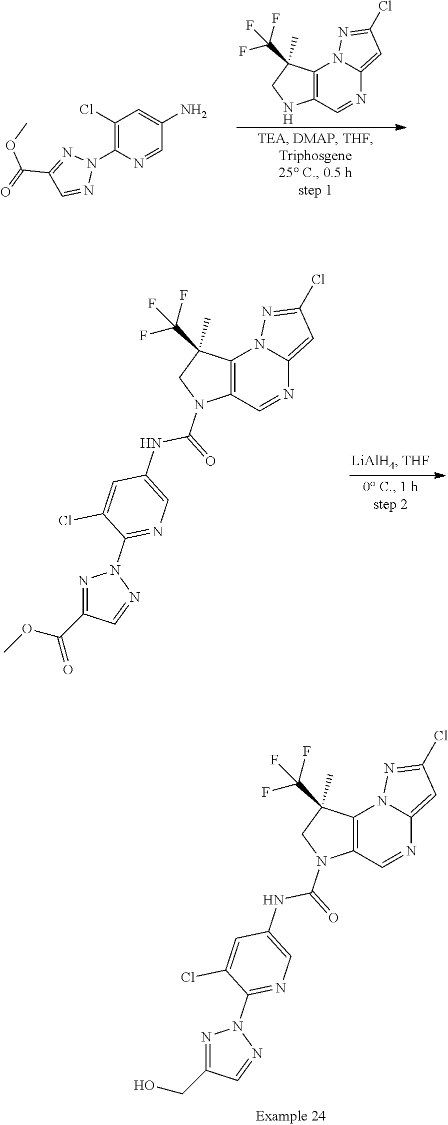 [SMILES: COC(=O)c1cnn(-c2ncc(N)cc2Cl)n1 ; C[C@@]1(C(F)(F)F)CNc2cnc3cc(Cl)nn3c21 ; COC(=O)c1cnn(-c2ncc(NC(=O)N3C[C@@](C)(C(F)(F)F)c4c3cnc3cc(Cl)nn43)cc2Cl)n1 ; C[C@@]1(C(F)(F)F)CN(C(=O)Nc2cnc(-n3ncc(CO)n3)c(Cl)c2)c2cnc3cc(Cl)nn3c21]

### Example 24: (R)-2-chloro-N-(5-chloro-6-(4-(hydroxymethyl)-2H-1,2,3-triazol-2-yl)pyridin-3-yi)-8-methyl-8-(trifluoromethyl)-7,8-dihydro-6H-pyrazolo[1,5-a]pyrrolo[2,3-e]pyrimidine-6-carboxamide

### Step 1: methyl (R)-2-(3-chloro-5-(2-chloro-8-methyl-8-(trifluoromethyl)-7,8-dihydro-6H-pyrazolo[1,5-a]pyrrolo[2,3-e]pyrimidine-6-carboxamido)pyridin-2-yl)-2H-1,2,3-triazole-4-carboxylate

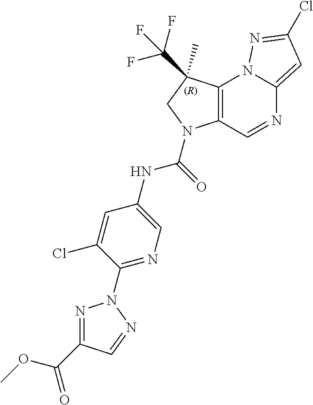

To a stirred solution of methyl 2-(5-amino-3-chloropyridin-2-yl)-2H-1,2,3-triazole-4-carboxylate (55 mg, 216.9 μmol) and triphosgene (80 mg, 271.1 μmol) in THF (5 mL) was added TEA (27 mg, 271.1 μmol, 38 uL) at 0° C. The resulting mixture was stirred for 0.5 h at 25° C. and then filtered. The resulting filtrate was added to a solution of Method M1 isomer 2 (50 mg, 180.7 μmol) in THF (2 mL). To this solution was then added TEA (183 mg, 1.8 mmol, 251.9 μL) and N,N-dimethylpyridin-4-amine (44 mg, 361.5 μmol). The mixture was stirred at 40° C. for 2 h. The resulting mixture was purified by Prep-TLC with MeOH/DCM (1/10) to give methyl (R)-2-(3-chloro-5-(2-chloro-8-methyl-8-(trifluoromethyl)-7,8-dihydro-6H-pyrazolo[1,5-a]pyrrolo[2,3-e]pyrimidine-6-carboxamido)pyridin-2-yl)-2H-1,2,3-triazole-4-carboxylate (70 mg, 35% yield) as a white solid. LC-MS: m/z 556 [M+H]⁺.

### Step 2: (R)-2-chloro-N-(5-chloro-6-(4-(hydroxymethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

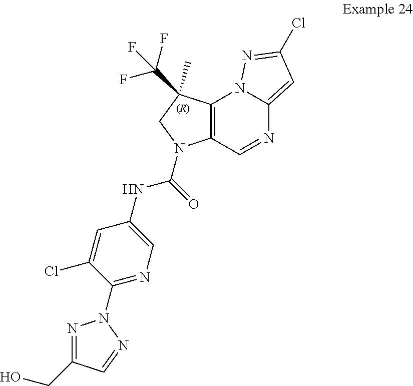

To a stirred solution of methyl (R)-2-(3-chloro-5-(2-chloro-8-methyl-8-(trifluoromethyl)-7,8-dihydro-6H-pyrazolo[1,5-a]pyrrolo[2,3-e]pyrimidine-6-carboxamido)pyridin-2-yl)-2H-1,2,3-triazole-4-carboxylate (35 mg, 31.4 μmol) in THF (1 mL) at 0° C. was added LiAlH₄ (1.4 mg, 37.7 μmol). The reaction mixture was stirred at 0° C. for 1 h. The resulting mixture was quenched with a saturated aqueous solution of ammonium chloride (1 mL), and the mixture was extracted with EtOAc (3×5 mL). The combined organic layers were dried over anhydrous Na₂SO₄ and concentrated under reduced pressure. The residue was purified by Prep-HPLC purification and the collected fractions were lyophilized to give (R)-2-chloro-N-(5-chloro-6-(4-(hydroxymethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide (1.4 mg, 8% yield) as a white solid. The enantiomer of Example 24 can be prepared analogously using Method M1 isomer 1.

Example 24: ¹H NMR (300 MHz, Chloroform-d) δ: 9.43 (s, 1H), 8.59 (s, 1H), 8.45 (s, 1H), 7.95 (s, 1H), 6.79 (s, 1H), 6.70 (s, 1H), 4.95 (s, 2H), 4.62 (d, J=10.1 Hz, 1H), 4.10 (d, J=10.0 Hz, 1H), 2.11 (s, 3H). LC-MS: m/z 528 [M+H]⁺.

### Example 25: (R)-2-chloro-N-(5-chloro-&(1-methyl-1H-pyrazol-3-yl)pyridin-3-yl)-8-methyl-8-(trifluoromethyl)-7,8-dihydro-6H-pyrazolo[1,5-a]pyrrolo[2,3-e]pyrimidine-6-carboxamide

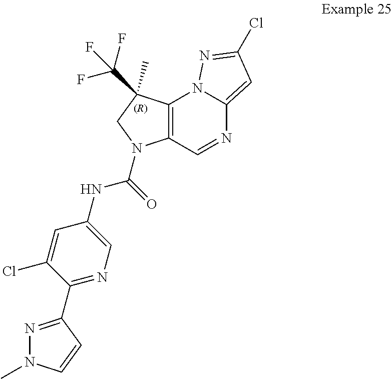

The title compound was prepared according to Method O1 Step 3 by using 5-chloro-6-(1-methyl-1H-pyrazol-3-yl)pyridin-3-amine and Method M1 isomer 2. The enantiomer of Example 25 can be prepared analogously using Method M1 isomer 1.

Example 25: ¹H NMR (300 MHz, DMSO-d₆) δ: 9.44 (s, 1H), 9.35 (s, 1H), 8.74 (d, J=2.1 Hz, 1H), 8.25 (d, J=2.1 Hz, 1H), 7.77 (d, J=2.4 Hz, 1H), 7.07 (s, 1H), 6.73 (d, J=2.4 Hz, 1H), 4.83 (d, J=11.7 Hz, 1H), 4.30 (d, J=11.7 Hz, 1H), 3.92 (s, 3H), 1.98 (s, 3H). LC-MS: m/z 511 [M+H]⁺.

### Example 26: (R)-2-chloro-N-(5-chloro-&(1H-pyrazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide

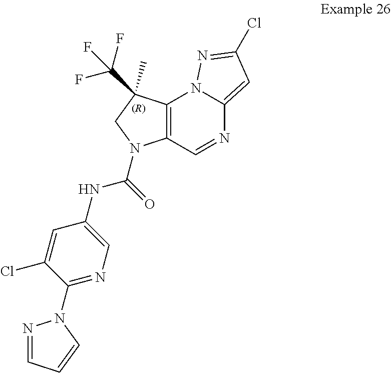

The title compound was prepared according to Method O1 Step 3 by using 5-chloro-6-(1H-pyrazol-1-yl)pyridin-3-amine and Method M isomer 2. The enantiomer of Example 26 can be prepared analogously using Method M1 isomer 1.

Example 26: ¹H NMR (300 MHz, DMSO-d₆) δ: 9.58 (s, 1H), 9.35 (s, 1H), 8.68 (d, J=2.4 Hz, 1H), 8.42 (d, J=2.4 Hz, 1H), 8.22 (d, J=2.1 Hz, 1H), 7.78 (d, J=2.1 Hz, 1H), 7.07 (s, 1H), 6.53-6.55 (m, 1H), 4.84 (d, J=11.7 Hz, 1H), 4.29 (d, J=11.7 Hz, 1H), 1.98 (s, 3H). LC-MS: m/z 497 [M+H]⁺.

### Example 27: (R)-2-chloro-8-methyl-8-(trifluoromethyl)-N-(2-(trifluoromethyl)pyridin-4-yl)-7,8-dihydro-6H-pyrazolo[1,5-a]pyrrolo[2,3-e]pyrimidine-6-carboxamide

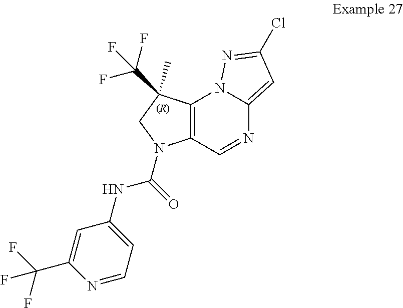

The title compound was prepared according to Method O1 Step 3 by using 2-(trifluoromethyl)pyridin-4-amine and Method M1 isomer 2. The enantiomer of Example 27 can be prepared analogously using Method M1 isomer 1.

Example 27: ¹H NMR (400 MHz, DMSO-d₆) δ: 9.74 (s, 1H), 9.34 (s, 1H), 8.63 (d, J=4.2 Hz, 1H), 8.11 (d, J=1.5 Hz, 1H), 7.88 (d, J=4.2 Hz, 1H), 7.08 (s, 1H), 4.87 (d, J=11.6 Hz, 1H), 4.29 (d, J=11.5 Hz, 1H), 1.97 (s, 3H). LC-MS: m/z 465 [M+H]⁺.

### Method Q1

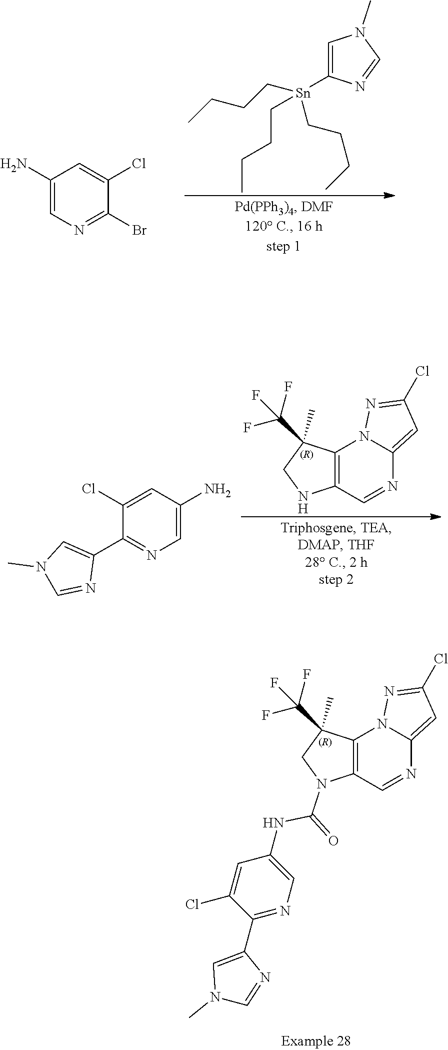

### Example 28: (R)-2-chloro-N-(5-chloro-6-(1-methyl-1H-imidazol-4-yl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 5-chloro-6-(1-methyl-1H-imidazol-4-yl)pyridin-3-amine

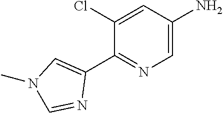

To a stirred solution of 6-bromo-5-chloro-pyridin-3-amine (200 mg, 964.1 μmol) in DMF (5 mL) were added 1-methyl-4-(tributylstannyl)-1H-imidazole (429 mg, 1.16 mmol) and Pd(PPh₃)₄ (111 mg, 96.3 μmol) under nitrogen. The reaction mixture was stirred at 120° C. for 16 h. The reaction mixture was quenched with water (10 mL). The resulting solution was extracted with EtOAc (3×5 mL). The combined organic layers were dried over anhydrous Na₂SO₄ and concentrated under vacuum. The residue was purified by Prep-HPLC to give 5-chloro-6-(1-methylimidazol-4-yl)pyridin-3-amine (70 mg, 31% yield) as a light-yellow solid. ¹H NMR (400 MHz, DMSO-d₆) δ: 9.12 (s, 1H), 8.19 (d, J=1.2 Hz, 1H), 8.04 (d, J=2.4 Hz, 1H), 7.14 (d, J=2.4 Hz, 1H), 3.89 (s, 3H), 2.74 (s, 2H). LC-MS: m/z 209 [M+H]⁺.

### Step 2: (R)-2-chloro-N-(5-chloro-6-(1-methyl-1H-imidazol-4-yl)pyridin-3-yl)-8-methyl-8-(trifluoromethyl)-7,8-dihydro-6H-pyrazolo[1,5-a]pyrrolo[2,3-e]pyrimidine-6-carboxamide

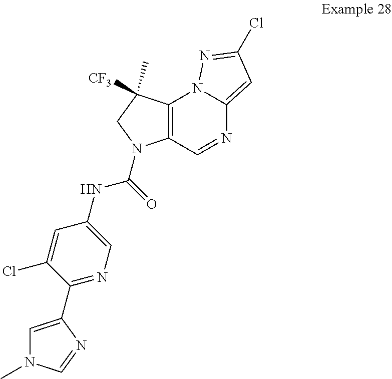 [SMILES: Cn1cnc(-c2ncc(NC(=O)N3C[C@@](C)(C(F)(F)F)c4c3cnc3cc(Cl)nn43)cc2Cl)c1]

The title compound was prepared according to Method M1 step 2 by using 5-chloro-6-(1-methylimidazol-4-yl)pyridin-3-amine and Method M1 isomer 2. The enantiomer of Example 28 can be prepared analogously using Method M1 isomer 1.

Example 28: ¹H NMR (300 MHz, DMSO-d₆) δ: 9.40 (s, 1H), 8.63 (s, 1H), 8.21 (d, J=2.4 Hz, 1H), 7.71 (s, 1H), 7.68 (s, 1H), 7.05 (s, 1H), 4.79 (d, J=11.7 Hz, 1H), 4.24 (d, J=11.7 Hz, 1H), 3.73 (s, 3H), 1.97 (s, 3H). LC-MS: m/z 511 [M+H]⁺.

### Method R¹

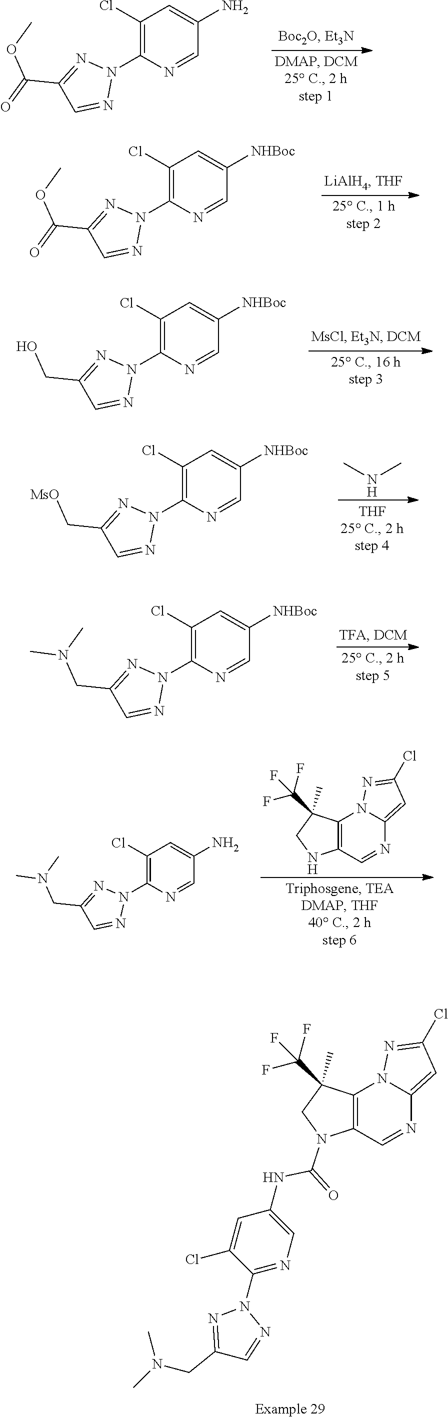

### Example 29: (R)-2-chloro-N-(5-chloro-6-(4-((dimethylamino)methyl)-2H-1,2,3-triazol-2-yl)pyridin-3-yl)-8-methyl-8-(trifluormethyl)-7,8-dihydro-6H-pyrazolo[15-a]pyrrolo[2,3-e]pyrimidine-6-carboxamide

### Step 1: methyl 2-(5-((tert-butoxycarbonyl)amino)-3-chloropyridin-2-yl)-2H-1,2,3-triazole-4-carboxylate

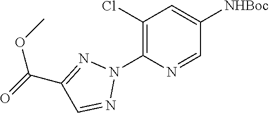 [SMILES: COC(=O)c1cnn(-c2ncc(NC(=O)OC(C)(C)C)cc2Cl)n1]

To a solution of methyl 2-(5-amino-3-chloropyridin-2-yl)-2H-1,2,3-triazole-4-carboxylate (500 mg, 2.0 mmol) in DCM (10 mL) was added TEA (598 mg, 5.91 mmol), N,N-dimethylpyridin-4-amine (24 mg, 197.1 μmol) and di-tert-butyl dicarbonate (516 mg, 2.4 mmol). The reaction mixture was stirred at 25° C. for 2 h. The reaction mixture was quenched with water (10 mL), and the aqueous phase was extracted with DCM (3×10 mL). The combined organic layers were dried over anhydrous Na₂SO₄ and concentrated under reduced pressure to give crude methyl 2-(5-((tert-butoxycarbonyl)amino)-3-chloropyridin-2-yl)-2H-1,2,3-triazole-4-carboxylate (600 mg, 51% yield) as a yellow solid which was used directly in next step. LC-MS: m/z 354 [M+H]⁺.

### Step 2: tert-butyl (5-chloro-6-(4-(hydroxymethyl)-2H-1,2,3-triazol-2-yl)pyridin-3-yl)carbamate

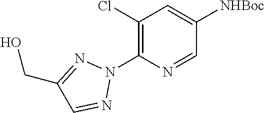 [SMILES: CC(C)(C)OC(=O)Nc1cnc(-n2ncc(CO)n2)c(Cl)c1]

To a stirred solution of methyl 2-(5-((tert-butoxycarbonyl)amino)-3-chloropyridin-2-yl)-2H-1,2,3-triazole-4-carboxylate (540 mg, 1.5 mmol) in THF (10 mL) was added LiAlH₄ (69 mg, 1.8 mmol). The reaction mixture was stirred at 25° C. for 1 h. The resulting mixture was quenched with water (10 mL), and the aqueous phase was extracted with EtOAc (3×20 mL). The combined organic layers were washed with brine, dried over anhydrous Na₂SO₄ and concentrated under reduced pressure. The residue was applied onto a silica gel column chromatography and eluted with EtOAc/PE (1:4) to give tert-butyl (5-chloro-6-(4-(hydroxymethyl)-2H-1,2,3-triazol-2-yl)pyridin-3-yl)carbamate (130 mg, 25% yield) as a white solid. LC-MS: m/z 326[M+H]⁺.

### Step 3: (2-(5-((tert-butoxycarbonyl)amino)-3-chloropyridin-2-yl)-2H-1,2,3-triazol-4-yl)methyl methanesulfonate

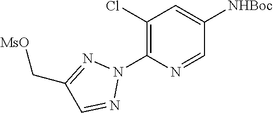 [SMILES: CC(C)(C)OC(=O)Nc1cnc(-n2ncc(COS(C)(=O)=O)n2)c(Cl)c1]

To a stirred solution of tert-butyl (5-chloro-6-(4-(hydroxymethyl)-2H-1,2,3-triazol-2-yl)pyridin-3-yl)carbamate (120 mg, 357.3 μmol) in DCM (1 mL) was added methanesulfonyl chloride (61 mg, 536.0 μmol) and TEA (108 mg, 1.1 mmol) slowly. The reaction mixture was stirred at 25° C. for 16 h. The resulting mixture was diluted with water (2 mL), and the mixture was extracted with DCM (3×3 mL). The combined organic layers were dried over anhydrous Na₂SO₄ and concentrated under reduced pressure to give crude (2-(5-((tert-butoxycarbonyl) amino)-3-chloropyridin-2-yl)-2H-1,2,3-triazol-4-yl)methyl methanesulfonate (100 mg, 66% yield) which was used directly in next step. LC-MS: m/z 404 [M+H]⁺.

### Step 4: tert-butyl (5-chloro-6-(4-((dimethylamino)methyl)-2H-1,2,3-triazol-2-yl)pyridin-3-yl)carbamate

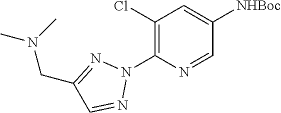 [SMILES: CN(C)Cc1cnn(-c2ncc(NC(=O)OC(C)(C)C)cc2Cl)n1]

To a stirred solution of (2-(5-((tert-butoxycarbonyl) amino)-3-chloropyridin-2-yl)-2H-1,2,3-triazol-4-yl) methyl methanesulfonate (90 mg, 211.7 μmol) in THF (5 mL) was added N-methylmethanamine (11 mg, 254.1 μmol). The reaction mixture was stirred at 25° C. for 2 h. The resulting mixture was diluted with water (5 mL), and the aqueous phase was extracted with EtOAc (3×5 mL). The combined organic layers were washed with brine, dried over anhydrous Na₂SO₄ and concentrated under reduced pressure to give tert-butyl (5-chloro-6-(4-((dimethylamino)methyl)-2H-1,2,3-triazol-2-yl)pyridin-3-yl)carbamate (100 mg, crude) as a white solid. LC-MS: m/z 353 [M+H]⁺.

### Step 5: 5-chloro-6-(4-((dimethylamino)methyl)-2H-1,2,3-triazol-2-yl)pyridin-3-amine

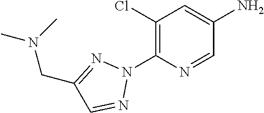 [SMILES: CN(C)Cc1cnn(-c2ncc(N)cc2Cl)n1]

To a stirred solution of tert-butyl (5-chloro-6-(4-((dimethylamino)methyl)-2H-1,2,3-triazol-2-yl)pyridin-3-yl)carbamate (100 mg, 260.8 μmol) in DCM (10 mL) was added 2,2,2-trifluoroacetic acid (297 mg, 2.6 mmol) slowly. The reaction mixture was stirred at 25° C. for 2 h. The pH of the mixture was adjusted to 7 with saturated aqueous solution of sodium bicarbonate, and it was extracted with DCM (3×10 mL). The combined organic layers were dried over anhydrous Na₂SO₄ and concentrated under reduced pressure. The residue was purified by Prep-TLC with MeOH/DCM (1:10) to afford 5-chloro-6-(4-((dimethylamino)methyl)-2H-1,2,3-triazol-2-yl)pyridin-3-amine (50 mg, 74% yield) as a white solid. LC-MS: m/z 253 [M+H]⁺.

### Step 6: (R)-2-chloro-N-(5-chloro-6-(4-((dimethylamino)methyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

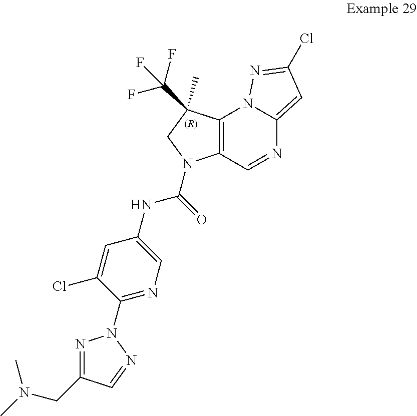

The title compound was prepared according to Method O1 Step 3 by using 5-chloro-6-(1-methylimidazol-4-yl)pyridin-3-amine and Method M1 isomer 2. The enantiomer of Example 29 can be prepared analogously using Method M1 isomer 1.

Example 29: ¹H NMR (300 MHz, DMSO-d₆) δ: 9.77 (s, 1H), 9.29 (s, 1H), 8.72 (d, J=2.4 Hz, 1H), 8.48 (d, J=2.4 Hz, 1H), 8.22 (s, 1H), 7.03 (s, 1H), 4.80 (d, J=11.7 Hz, 1H), 4.48 (s, 2H), 4.23 (d, J=11.4 Hz, 1H), 2.75 (s, 6H), 1.92 (s, 3H). LC-MS: m/z 555 [M+H]⁺.

### Method S1

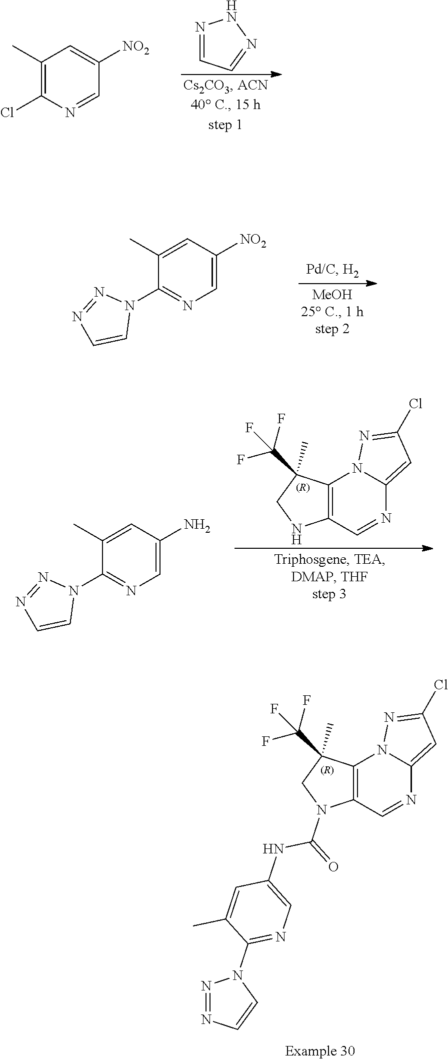

### Example 30: (R)-2-chloro-8-methyl-N-(5-methyl-6-(1H-1,2,3-triazol-1-yl)pyridin-3-yl)-8-(trifluoromethyl)-7,8-dihydro-6H-pyrazolo[1,5-a]pyrrolo[2,3-e]pyrimidine-6-carboxamide

### Step 1: 3-methyl-5-nitro-2-(1H-1,2,3-triazol-1-yl)pyridine

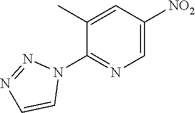

To a stirred solution of 2-chloro-3-methyl-5-nitropyridine (2 g, 11.6 mmol) in ACN (30 mL) were added 2H-1,2,3-triazole (880 mg, 12.8 mmol) and Cs₂CO₃ (2.1 g, 15.1 mmol). The reaction was stirred at 40° C. for 15 h. LCMS showed the reaction was complete. The solvent was removed under vacuum. The residue was diluted with water (50 mL) and extracted with EtOAc (2×50 mL). The combined organic layers were concentrated under vacuum. The crude product was purified by Prep-HPLC to give 3-methyl-5-nitro-2-(1H-1,2,3-triazol-1-yl)pyridine (300 mg, 12% yield) as a white solid. ¹H NMR (400 MHz, DMSO-d₆) δ: 9.24 (d, J=2.4 Hz, 1H), 8.87 (d, J=2.4 Hz, 1H), 7.78 (d, J=1.2 Hz, 1H), 8.03 (d, J=1.2 Hz, 1H), 2.58 (s, 3H). LC-MS: m/z 206 [M+H]⁺.

### Step 2: 5-methyl-6-(1H-1,2,3-triazol-1-yl)pyridin-3-amine

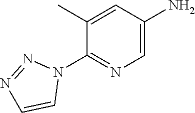

To a stirred solution of 3-methyl-5-nitro-2-(1H-1,2,3-triazol-1-yl)pyridine (100 mg, 487.4 μmol) in MeOH (10 mL) were added Pd/C (20 mg, 10%). The flask was evacuated and flushed three times with nitrogen, followed by flushing with hydrogen. The reaction was stirred at 25° C. for 1 h under an atmosphere of hydrogen. LCMS showed the reaction was completed. The solid was filtered out. The filtrate was concentrated under vacuum. This resulted in 5-methyl-6-(1H-1,2,3-triazol-1-yl)pyridin-3-amine (62 mg, 65% yield) as a white solid. ¹H NMR (400 MHz, DMSO-d₆) δ: 8.36 (d, J=1.2 Hz, 1H), 7.85 (d, J=0.8 Hz, 1H), 7.70 (d, J=2.4 Hz, 1H), 6.96 (d, J=2.4 Hz, 1H), 5.73 (s, 2H), 2.06 (s, 3H). LC-MS: m/z 176 [M+H]⁺.

### Step 3: (R)-2-chloro-8-methyl-N-(5-methyl-6-(1H-1,2,3-triazol-1-yl)pyridin-3-yl)-8-(trifluoromethyl)-7,8-dihydro-6H-pyrazolo[1,5-a]pyrrolo[2,3-e]pyrimidine-6-carboxamide

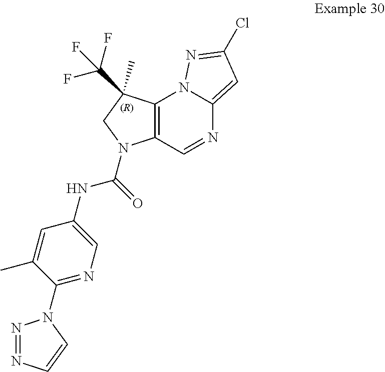

The title compound was prepared according to Method O1 Step 3 by using 5-methyl-6-(1H-1,2,3-triazol-1-yl)pyridin-3-amine and Method M1 isomer 2. The enantiomer of Example 30 can be prepared analogously using Method M1 isomer 1.

Example 30: ¹H NMR (400 MHz, DMSO-d₆) δ: 9.48 (s, 1H), 9.36 (s, 1H), 8.60-8.64 (m, 2H), 8.20 (d, J=2.0 Hz, 1H), 7.97 (d, J=0.8 Hz, 1H), 7.08 (s, 1H), 4.86 (d, J=11.6 Hz, 1H), 4.30 (d, J=11.5 Hz, 1H), 2.33 (s, 3H), 1.99 (s, 3H). LC-MS: m/z 478 [M+H]⁺.

### Method T1

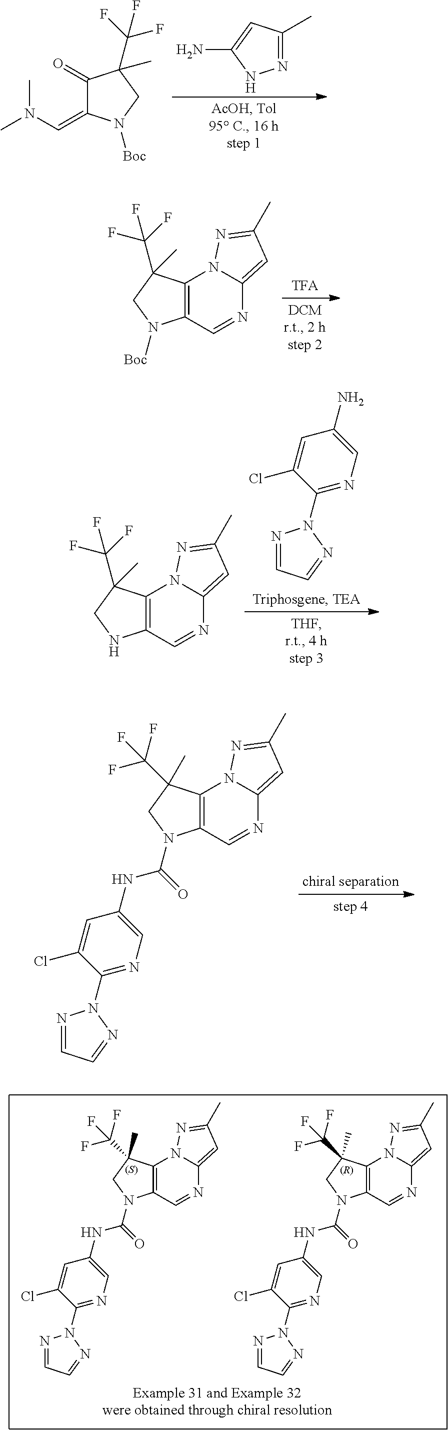

### Examples 31 and 32: Single enantiomers obtained from a racemic mixture containing (S)—N-(5-chloro)-6-(2H-1,2,3-triazol-2-yl)pyridin-3-yl)-2,8-dimethyl-8-(trifluoromethyl)-7,8-dihydro-6H-pyrazolo[1,5-a]pyrrolo[2,3-e]pyrimidine-6-carboxamide and (R)—N-(5-chloro-6-(2H-1,2,3-triazol-2-yl)pyridin-3-yl)-2,8-dimethyl-8-(trifluoromethyl)-7,8-dihydro-6H-pyrazolo[1,5-a]pyrrolo[2,3-e]pyrimidine-6-carboxamide

### Step 1: tert-butyl 2,8-dimethyl-8-(trifluoromethyl)-7,8-dihydro-6H-pyrazolo[1,5-a]pyrrolo[2,3-e]pyrimidine-6-carboxylate

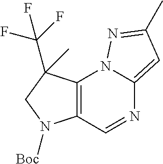 [SMILES: Cc1cc2ncc3c(n2n1)C(C)(C(F)(F)F)CN3C(=O)OC(C)(C)C]

To a solution of tert-butyl (E)-2-((dimethylamino)methylene)-4-methyl-3-oxo-4-(trifluoromethyl)pyrrolidine-1-carboxylate (Method K1 step 8; 500 mg, 1.5 mmol) in toluene (10 mL) and AcOH (1 mL) was added 3-methyl-1H-pyrazol-5-amine (1.2 g, 1.5 mmol) under N₂. The resulting mixture was stirred for 16 h at 95° C. The reaction mixture was quenched with water (100 mL) and extracted with EtOAc (3×100 mL). The combined organic layers were dried over anhydrous Na₂SO₄ and concentrated under vacuum. The residue was applied onto a silica gel column chromatography and eluted with EtOAc/PE (1:3) to give tert-butyl 2,8-dimethyl-8-(trifluoromethyl)-7,8-dihydro-6H-pyrazolo[1,5-a]pyrrolo[2,3-e]pyrimidine-6-carboxylate (300 mg, 53% yield) as a yellow oil. LC-MS: m/z 357 [M+H]⁺.

### Step 2: 2,8-dimethyl-8-(trifluoromethyl)-7,8-dihydro-6H-pyrazolo[1,5-a]pyrrolo[2,3-e]pyrimidine

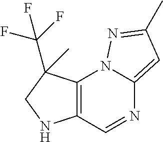 [SMILES: Cc1cc2ncc3c(n2n1)C(C)(C(F)(F)F)CN3]

To a solution of tert-butyl 2,8-dimethyl-8-(trifluoromethyl)-7,8-dihydro-6H-pyrazolo[1,5-a]pyrrolo[2,3-e]pyrimidine-6-carboxylate (300 mg, 841.8 μmol) in DCM (8 mL) was added TFA (2 mL). The resulting mixture was stirred for 2 h at room temperature and concentrated under vacuum. To the residue was added water (50 mL) and the resulting mixture was extracted with EtOAc (3×50 mL). The combined organic layers were dried over anhydrous Na₂SO₄ and concentrated under vacuum. The residue was applied onto a silica gel column chromatography and eluted with EtOAc/PE (1:3) to give 2,8-dimethyl-8-(trifluoromethyl)-7,8-dihydro-6H-pyrazolo[1,5-a]pyrrolo[2,3-e]pyrimidine (180 mg, 76% yield) as a yellow solid. LC-MS: m/z 257 [M+H]⁺.

### Step 3: N-(5-chloro-6-(2H-1,2,3-triazol-2-yl)pyridin-3-yl)-2,8-dimethyl-8-(trifluoromethyl)-7,8-dihydro-6H-pyrazolo[1,5-a]pyrrolo[2,3-e]pyrimidine-6-carboxamide

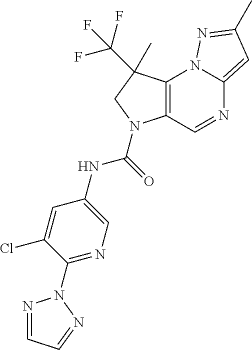 [SMILES: Cc1cc2ncc3c(n2n1)C(C)(C(F)(F)F)CN3C(=O)Nc1cnc(-n2nccn2)c(Cl)c1]

To a solution of 5-chloro-6-(2H-1,2,3-triazol-2-yl)pyridin-3-amine (Method A1 step 2; 80 mg, 351.2 μmol) in THF (8 mL) were added triphosgene (62 mg, 210.7 μmol) and TEA (46 mg, 456 μmol) at 25° C. The resulting mixture was stirred for 1 h at 25° C. and then filtered. The resulting filtrate was added to a solution of 2,8-dimethyl-8-(trifluoromethyl)-7,8-dihydro-6H-pyrazolo[1,5-a]pyrrolo [2,3-e]pyrimidine (90 mg, 351.2 μmol) in THF (1 mL). The reaction was stirred at room temperature for 4 h. The reaction mixture was quenched with water (50 mL) and extracted with EtOAc (3×50 mL). The combined organic layers were dried over anhydrous Na₂SO₄ and concentrated under vacuum. The residue was purified by silica gel column chromatography, eluting with EtOAc/PE (1:1) to give N-(5-chloro-6-(2H-1,2,3-triazol-2-yl)pyridin-3-yl)-2,8-dimethyl-8-(trifluoromethyl)-7,8-dihydro-6H-pyrazolo[1,5-a]pyrrolo[2,3-e]pyrimidine-6-carboxamide (30 mg, 19% yield) as a white solid. ¹H NMR (300 MHz, DMSO-d₆) δ: 9.66 (s, 1H), 9.24 (s, 1H), 8.75 (d, J=2.4 Hz, 1H), 8.51 (d, J=2.4 Hz, 1H), 8.17 (s, 2H), 6.69 (s, 1H), 4.83 (d, J=11.4 Hz, 1H), 4.29 (d, J=11.4 Hz, 1H), 2.46-2.51 (m, 3H), 2.01 (s, 3H); LC-MS: m/z 478 [M+H]⁺.

### Step 4: Separation of enantiomers to obtain (S)—N-(5-chloro-6-(2H-1,2,3-triazol-2-yl)pyridin-3-yl)-2,8-dimethyl-8-(trifluoromethyl)-7,8-dihydro-6H-pyrazolo[1,5-a]pyrrolo[2,3-e]pyrimidine-6-carboxamide and (R)—N-(5-chloro-6-(2H-1,2,3-triazol-2-yl)pyridin-3-yl)-2,8-dimethyl-8-(trifluoromethyl)-7,8-dihydro-6H-pyrazolo[1,5-a]pyrrolo[2,3-e]pyrimidine-6-carboxamide

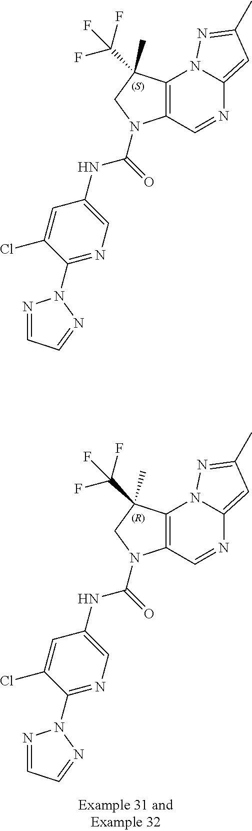

30 mg of N-(5-chloro-6-(2H-1,2,3-triazol-2-yl)pyridin-3-yl)-2,8-dimethyl-8-(trifluoromethyl)-7,8-dihydro-6H-pyrazolo[1,5-a]pyrrolo[2,3-e]pyrimidine-6-carboxamide was submitted to chiral HPLC purification (Column: CHIRALPAK IA, 2×25 cm, 5 um; Mobile Phase A: Hex (0.5% 2M NH₃-MeOH)—HPLC, Mobile Phase B: EtOH—HPLC; Flow rate: 20 mL/min; isocraitic 10% B; 220/254 nm; RTL: 15.722; RT2: 21.47; Injection Volume: 1.2 ml; Number of Runs: 4). The first eluting isomer (RT 15.72 min) was concentrated and lyophilized to afford Example 31 as a white solid (7.1 mg, 25% yield). The second eluting isomer (RT 21.47 min) was concentrated and lyophilized to afford Example 32 as a white solid (9.2 mg, 32% yield).

Example 31: ¹H NMR (300 MHz, DMSO-d₆) δ: 9.66 (s, 1H), 9.24 (s, 1H), 8.75 (d, J=2.4 Hz, 1H), 8.51 (d, J=2.4 Hz, 1H), 8.17 (s, 2H), 6.69 (s, 1H), 4.84 (d, J=11.4 Hz, 1H), 4.29 (d, J=11.4 Hz, 1H), 2.46-2.51 (m, 3H), 2.01 (s, 3H). LC-MS: m/z 478 [M+H]⁺.

Example 32: ¹H NMR (300 MHz, DMSO-d₆) δ: 9.66 (s, 1H), 9.24 (s, 1H), 8.75 (d, J=2.4 Hz, 1H), 8.51 (d, J=2.4 Hz, 1H), 8.17 (s, 2H), 6.69 (s, 1H), 4.83 (d, J=11.4 Hz, 1H), 4.30 (d, J=11.4 Hz, 1H), 2.46-2.51 (m, 3H), 2.01 (s, 3H). LC-MS: m/z 478 [M+H]⁺.

### Method U1

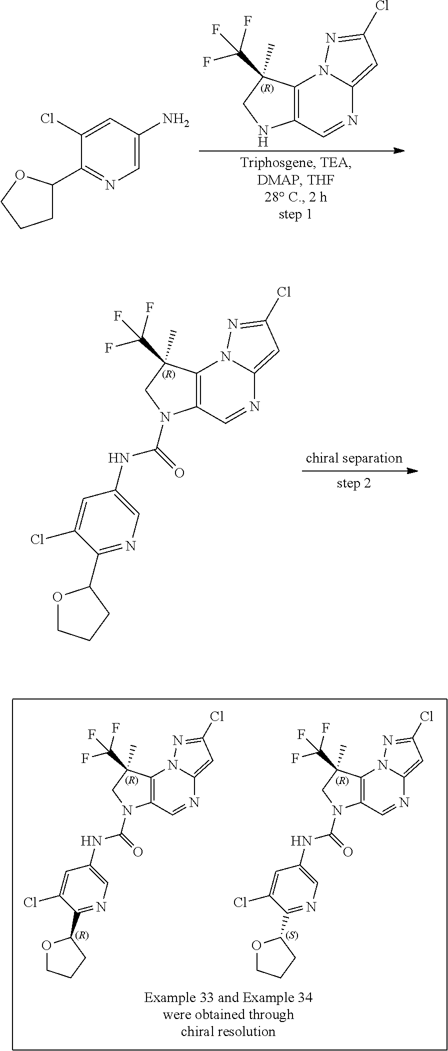

### Examples 33 and 34: Single stereoisomers obtained from a mixture containing (R)-2-chloro-N-(5-chloro-6-((R)-tetrahydrofuran-2-yl)pyridin-3-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(5-chloro-6-((S)-tetrahydrofuran-2-yl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: (8R)-2-chloro-N-(5-chloro-6-(tetrahydrofuran-2-yl)pyridin-3-yl)-8-methyl-8-(trifluoromethyl)-7,8-dihydro-6H-pyrazolo[1,5-a]pyrrolo[2,3-e]pyrimidine-6-carboxamide

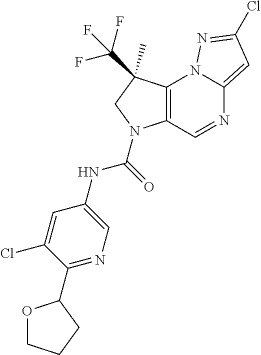 [SMILES: C[C@@]1(C(F)(F)F)CN(C(=O)Nc2cnc(C3CCCO3)c(Cl)c2)c2cnc3cc(Cl)nn3c21]

To a stirred solution of Method M1 isomer 2 (40 mg, 144.6 μmol) and triphosgene (26 mg, 86.7 μmol) in THF (3 mL) was added TEA (22 mg, 216.9 μmol, 30 uL) at 0° C. The mixture was stirred at 28° C. for 0.5 h then filtered. The resulting filtrate was added to a solution of 5-chloro-6-tetrahydrofuran-2-yl-pyridin-3-amine (29 mg, 144.6 μmol) in THF (1 mL). To this solution was then added TEA (146 mg, 1.4 mmol, 201.5 μL) and N,N-dimethylpyridin-4-amine (35 mg, 289.2 μmol). The mixture was stirred at 28° C. for 2 h. The solvent was removed in vacuo and the residue was purified by Prep-TLC with MeOH/DCM (10:1) to give (8R)-2-chloro-N-(5-chloro-6-(tetrahydrofuran-2-yl)pyridin-3-yl)-8-methyl-8-(trifluoromethyl)-7,8-dihydro-6H-pyrazolo[1,5-a]pyrrolo[2,3-e]pyrimidine-6-carboxamide (18 mg, 25% yield) as a yellow solid. LC-MS: m/z 501 [M+H]⁺.

### Step 2: Separation of stereoisomers to obtain (R)-2-chloro-N-(5-chloro-6-((R)-tetrahydrofuran-2-yl)pyridin-3-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(5-chloro-6-((S)-tetrahydrofuran-2-yl)pyridin-3-yl)-8-methyl-8-(trifluoromethyl)-7,8-dihydro-6H-pyrazolo[1,5-a]pyrrolo[2,3-e]pyrimidine-6-carboxamide

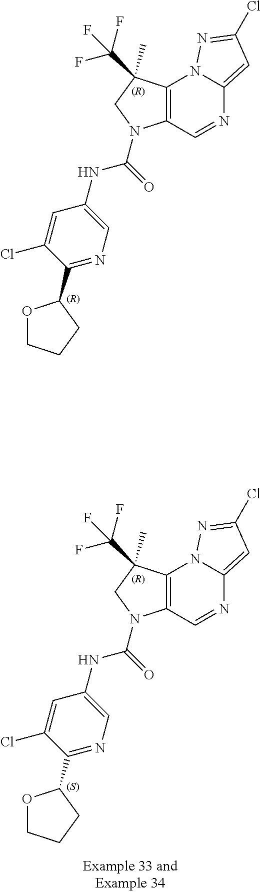

15 mg of (8R)-2-chloro-N-(5-chloro-6-(tetrahydrofuran-2-yl)pyridin-3-yl)-8-methyl-8-(trifluoromethyl)-7,8-dihydro-6H-pyrazolo[1,5-a]pyrrolo[2,3-e]pyrimidine-6-carboxamide was submitted to chiral HPLC purification (Column: CHIRAL ART Cellulose-SB, 2×25 cm, 5 um; Mobile Phase A: Hex (0.5% 2M NH₃-MeOH)—HPLC, Mobile Phase B: EtOH-HPLC; Flow rate: 20 mL/min; Gradient: 30 B to 30 B in 20 min; 220/254 nm; RT1: 10.1; RT2: 16.62; Injection Volume: 2 ml; Number of Runs: 1). The first eluting isomer (RT 10.1 min) was concentrated and lyophilized to afford Example 33 (3.9 mg, 52% yield) as white solid. The second eluting isomer (RT 16.62 min) was concentrated and lyophilized to afford Example 34 (1.5 mg, 20% yield) as a white solid. The corresponding enantiomers of Example 33 and Example 34 can be prepared analogously using Method M1 isomer 1.

Example 33: ¹H NMR (300 MHz, DMSO-d₆) δ: 9.42 (s, 1H), 9.34 (s, 1H), 8.67 (d, J=2.1 Hz, 1H), 8.17 (d, J=2.1 Hz, 1H), 7.07 (s, 1H), 5.24 (t, J=6.9 Hz, 1H), 4.82 (d, J=11.4 Hz, 1H), 4.26 (d, J=12.0 Hz, 1H), 3.80-3.94 (m, 2H), 1.98-2.23 (m, 4H), 1.98 (s, 3H). LC-MS: m/z 501 [M+H]⁺.

Example 34: ¹H NMR (300 MHz, DMSO-d₆) δ: 9.42 (s, 1H), 9.34 (s, 1H), 8.67 (d, J=2.1 Hz, 1H), 8.18 (d, J=2.1 Hz, 1H), 7.07 (s, 1H), 5.24 (t, J=6.9 Hz, 1H), 4.82 (d, J=11.4 Hz, 1H), 4.26 (d, J=11.4 Hz, 1H), 3.78-3.96 (m, 2H), 1.95-2.28 (m, 4H), 1.96 (s, 3H). LC-MS: m/z 501 [M+H]⁺.

### Method V1

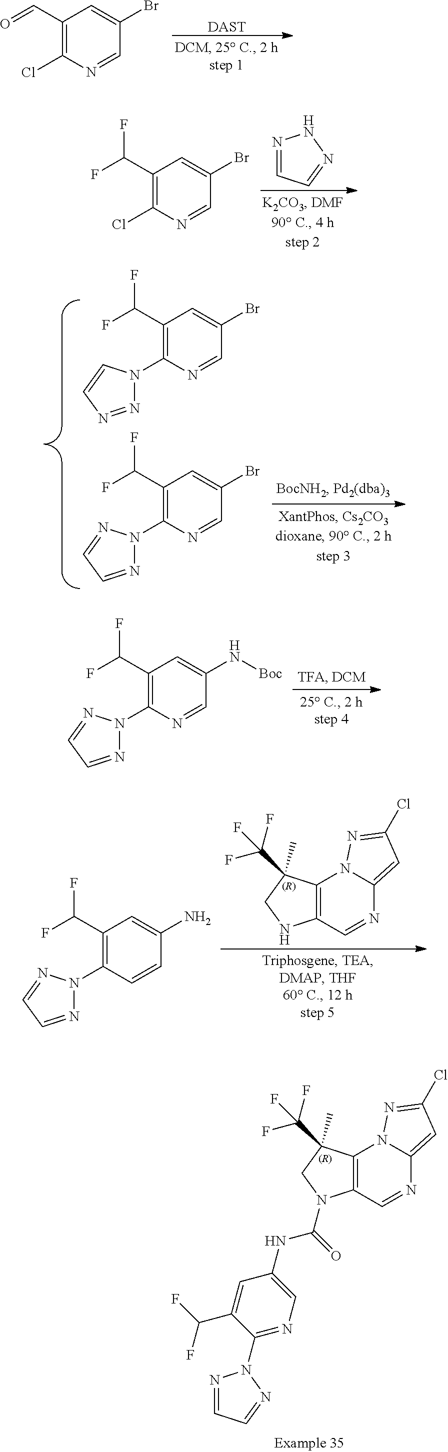

### Example 35: (R)-2-chloro-N-(5-(difluoromethyl)-6-(2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 5-bromo-2-chloro-3-(difluoromethyl)pyridine

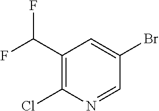 [SMILES: FC(F)c1cc(Br)cnc1Cl]

To a stirred solution of 5-bromo-2-chloronicotinaldehyde (2.5 g, 11.3 mmol) in DCM (50 mL) was added DAST (3.6 g, 22.6 mmol) dropwise at 0° C. The reaction mixture was stirred at 25° C. for 2 h. The pH of the mixture was adjusted to 8 with saturated aqueous NaHCO₃. The resulting mixture was extracted with DCM (3×100 mL). The combined organic layers were washed with brine (100 mL), dried over anhydrous Na₂SO₄, filtered and concentrated under reduced pressure to afford crude 5-bromo-2-chloro-3-(difluoromethyl)pyridine (1.5 g, 55% yield) as a light-yellow oil. ¹H NMR (400 MHz, Chloroform-d) δ: 8.55-8.59 (m, 1H), 8.09-8.14 (m, 1H), 6.87 (t, J=54.0 Hz, 1H). LC-MS: m/z 242 [M+H]⁺.

### Step 2: 5-bromo-3-(difluoromethyl)-2-(2H-1,2,3-triazol-2-yl)pyridine and 5-bromo-3-(difluoromethyl)-2-(1H-1,2,3-triazol-1-yl)pyridine

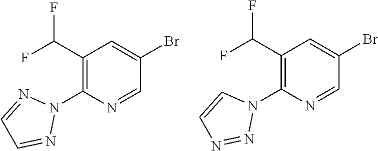 [SMILES: FC(F)c1cc(Br)cnc1-n1nccn1 ; FC(F)c1cc(Br)cnc1-n1ccnn1]

To a stirred solution of 5-bromo-2-chloro-3-(difluoromethyl)pyridine (1.5 g, 6.2 mmol) in DMF (20 mL) were added K₂CO₃ (1.7 g, 12.8 mmol) and 2H-1,2,3-triazole (512 mg, 7.4 mmol). The reaction mixture was stirred at 90° C. for 4 h. The mixture was poured into water (30 mL) and extracted with EtOAc (3×100 mL). The combined organic layers were washed with brine (3×100 mL), dried over anhydrous Na₂SO₄. After filtration, the filtrate was concentrated under reduced pressure. The residue was applied onto a silica gel column and eluting with EtOAc/PE (1:3) to afford a mixture of 5-bromo-3-(difluoromethyl)-2-(2H-1,2,3-triazol-2-yl)pyridine and 5-bromo-3-(difluoromethyl)-2-(1H-1,2,3-triazol-1-yl)pyridine (1.6 g, 94% yield) as a yellow solid. LC-MS: m/z 275 [M+H]⁺.

### Step 3: tert-butyl (5-(difluoromethyl)-6-(2H-1,2,3-triazol-2-yl)pyridin-3-yl)carbamate and tert-butyl (5-(difluoromethyl)-6-(1H-1,2,3-triazol-1-yl)pyridin-3-yl)carbamate

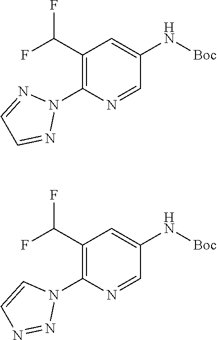 [SMILES: CC(C)(C)OC(=O)Nc1cnc(-n2nccn2)c(C(F)F)c1 ; CC(C)(C)OC(=O)Nc1cnc(-n2ccnn2)c(C(F)F)c1]

To a stirred solution of the mixture of 5-bromo-3-(difluoromethyl)-2-(2H-1,2,3-triazol-2-yl)pyridine and 5-bromo-3-(difluoromethyl)-2-(1H-1,2,3-triazol-1-yl)pyridine (1.6 g, 5.8 mmol) in dioxane (160 mL) were added tert-butyl carbamate (1.02 g, 8.7 mmol), xantphos (1.01 g, 1.7 mmol), Pd₂(dba)₃ (668 mg, 1.2 mmol) and Cs₂CO₃ (5.7 g, 17.4 mmol). The reaction mixture was stirred at 90° C. for 2 h under N₂. The mixture was allowed to cool down to room temperature. The resulting mixture was filtered. The filter cake was washed with EtOAc (3×100 mL). The filtrate was concentrated under reduced pressure. The residue was applied onto a silica gel column and eluted with EtOAc/PE (1:4) to afford tert-butyl (5-(difluoromethyl)-6-(2H-1,2,3-triazol-2-yl)pyridin-3-yl)carbamate (700 mg, 38.7% yield) as a yellow solid and tert-butyl (5-(difluoromethyl)-6-(1H-1,2,3-triazol-1-yl)pyridin-3-yl)carbamate (600 mg, 33% yield) as a yellow solid.

Tert-butyl (5-(difluoromethyl)-6-(2H-1,2,3-triazol-2-yl)pyridin-3-yl)carbamate: ¹H NMR (400 MHz, Methanol-d₄) δ: 8.69 (d, J=2.4 Hz, 1H), 8.50 (d, J=2.4 Hz, 1H), 8.04 (s, 2H), 7.45 (t, J=54.8 Hz, 1H), 1.55 (s, 9H). LC-MS: m/z 312 [M+H]⁺

Tert-butyl (5-(difluoromethyl)-6-(1H-1,2,3-triazol-1-yl)pyridin-3-yl)carbamate: ¹H NMR (400 MHz, Methanol-d₄) δ: 8.72 (d, J=2.4 Hz, 1H), 8.59 (d, J=1.2 Hz, 1H), 8.50 (d, J=2.4 Hz, 1H), 7.91 (d, J=1.2 Hz, 1H), 7.60 (t, J=54.8 Hz, 1H), 1.56 (s, 9H). LC-MS: m/z 312 [M+H]⁺.

### Step 4: 5-(difluoromethyl)-6-(2H-1,2,3-triazol-2-yl)pyridin-3-amine

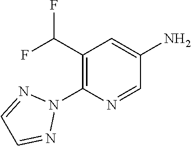 [SMILES: Nc1cnc(-n2nccn2)c(C(F)F)c1]

To a stirred solution of tert-butyl (5-(difluoromethyl)-6-(2H-1,2,3-triazol-2-yl)pyridin-3-yl)carbamate (200 mg, 643 μmol) in DCM (20 mL) was added TFA (2.9 g, 25.7 mmol). The mixture was stirred at 25 for 2 h. The resulting mixture was concentrated under vacuum. The residue was applied onto a silica gel column chromatography and eluted with EtOAc/PE (1:1) to afford 5-(difluoromethyl)-6-(2H-1,2,3-triazol-2-yl)pyridin-3-amine (110 mg, 81% yield) as a yellow solid. ¹H NMR (300 MHz, Methanol-d₄) δ: 8.00 (d, J=2.7 Hz, 1H), 7.96 (s, 2H), 7.08 (t, J=55.2 Hz, 1H). LC-MS: m/z 212 [M+H]⁺.

### Step 5: (R)-2-chloro-N-(5-(difluoromethyl)-6-(2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

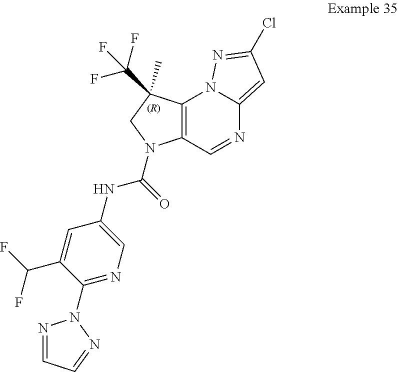

To a stirred solution of Method M1 isomer 2 (13 mg, 43 μmol) in THF (4 mL) was added triphosgene (13 mg, 43 μmol) and TEA (11 mg, 108 μmol) at 0° C. The resulting mixture was stirred for 0.5 h at 25° C. and then filtered. The resulting filtrate was added to a solution of 5-(difluoromethyl)-6-(2H-1,2,3-triazol-2-yl)pyridin-3-amine (18 mg, 86 μmol) in THF (1 mL). To this solution was then added TEA (73 mg, 722 μmol) and N,N-dimethylpyridin-4-amine (18 mg, 144 μmol). The mixture was stirred at 60° C. for 12 h. The mixture was poured into water (40 mL) and extracted with EtOAc (3×40 mL). The combined organic layers were washed with brine (50 mL), dried over anhydrous Na₂SO₄ and concentrated under vacuum. The residue was submitted to Prep-HPLC purification and the collected fractions were lyophilized to give (R)-2-chloro-N-(5-(difluoromethyl)-6-(2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide (15.8 mg, 42% yield) as a white solid. The enantiomer of Example 35 can be prepared analogously using Method M1 isomer 1.

Example 35: ¹H NMR (300 MHz, DMSO-d₆) δ: 9.70 (s, 1H), 9.38 (s, 1H), 8.97 (d, J=2.4 Hz, 1H), 8.60 (d, J=2.4 Hz, 1H), 8.23 (s, 2H), 7.36 (t, J=54.3 Hz, 1H), 7.08 (s, 1H), 4.88 (d, J=11.4 Hz, 1H), 4.32 (d, J=11.4 Hz, 1H), 1.99 (s, 3H). LC-MS: m/z 514 [M+H]⁺.

### Method W1

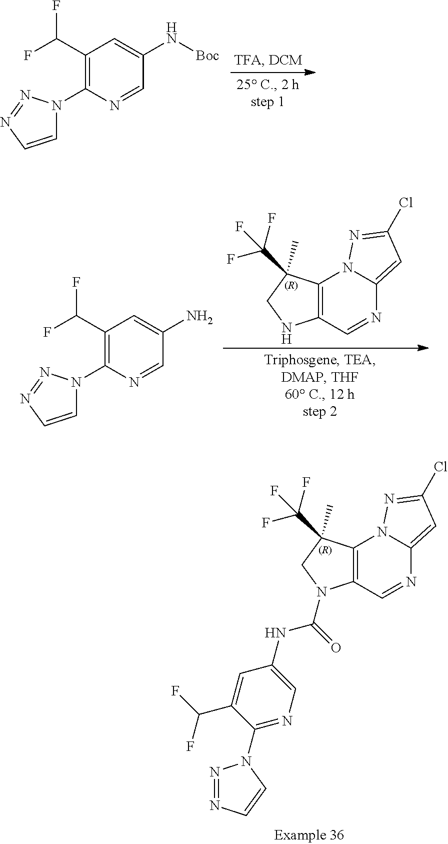

### Example 36: (R)-2-chloro-N-(5-(difluoromethyl)-6-(1H-1,2,3-triazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 5-(difluoromethyl)-6-(1H-1,2,3-triazol-1-yl)pyridin-3-amine

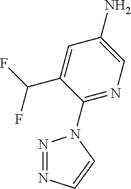 [SMILES: Nc1cnc(-n2ccnn2)c(C(F)F)c1]

To a stirred solution of tert-butyl (5-(difluoromethyl)-6-(1H-1,2,3-triazol-1-yl)pyridin-3-yl)carbamate (Method V1 Step 2; 200 mg, 642 μmol) in DCM (20 mL) was added TFA (2.9 g, 25.7 mmol). The mixture was stirred at r.t. for 2 h. The resulting mixture was concentrated under reduced pressure. The residue was applied onto a silica gel column chromatography and eluted with EtOAc/PE (1:1) to afford 5-(difluoromethyl)-6-(1H-1,2,3-triazol-1-yl)pyridin-3-amine (110 mg, 81% yield) as a yellow solid. ¹H NMR (300 MHz, Methanol-d₄) δ: 8.41 (d, J=1.2 Hz, 1H), 8.00-8.04 (m, 1H), 7.87 (d, J=1.2 Hz, 1H), 7.46 (d, J=3.0 Hz, 1H), 7.08 (t, J=54.9 Hz, 1H). LC-MS: m/z 212 [M+H]⁺.

### Step 2: (R)-2-chloro-N-(5-(difluoromethyl)-6-(1H-1,2,3-triazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide

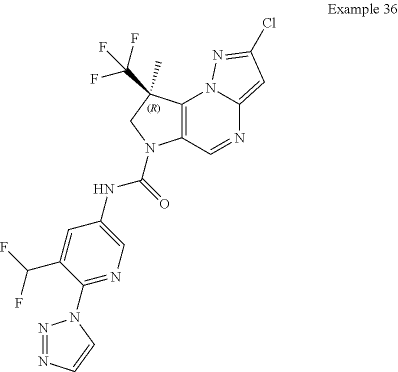

The title compound was prepared according to Method V1 Step 5 by using 5-(difluoromethyl)-6-(1H-1,2,3-triazol-1-yl)pyridin-3-amine and Method M1 isomer 2. The enantiomer of Example 36 can be prepared analogously using Method M1 isomer 1.

Example 36: ¹H NMR (300 MHz, DMSO-d₆) δ: 9.70 (s, 1H), 9.38 (s, 1H), 8.99 (d, J=2.4 Hz, 1H), 8.80 (d, J=1.2 Hz, 1H), 8.62 (d, J=2.4 Hz, 1H), 8.04 (d, J=2.4 Hz, 1H), 7.43 (t, J=54.0 Hz, 1H), 7.09 (s, 1H), 4.88 (d, J=11.4 Hz, 1H), 4.32 (d, J=11.4 Hz, 1H), 2.00 (s, 3H). LC-MS: m/z 514 [M+H]⁺.

### Example 37: (R)-2-chloro-N-(4,4-difluorocyclohexyl)-8-methyl-8-(trifluoromethyl)-7,8-dihydro-6H-pyrazolo[1,5-a]pyrrolo[2,3-e]pyrimidine-6-carboxamide

### Step 1: (R)-2-chloro-N-(4,4-difluorocyclohexyl)-8-methyl-8-(trifluoromethyl)-7,8-dihydro-6H-pyrazolo[1,5-a]pyrrolo[2,3-e]pyrimidine-6-carboxamide

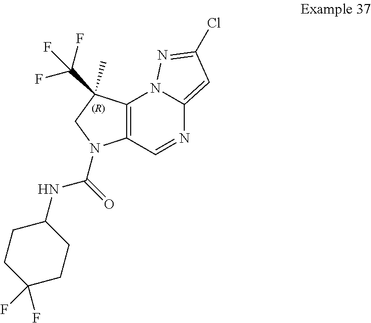

The title compound was prepared according to Method M1 step 2 by using 4,4-difluorocyclohexan-1-amine hydrochloride and Method M1 isomer 2. The enantiomers of the diastereomeric pair in Example 37 can be prepared analogously using Method M1 isomer 1.

Example 37: ¹H NMR (300 MHz, DMSO-d₆) δ: 9.30 (s, 1H), 7.00 (s, 1H), 6.91 (d, J=7.5 Hz, 1H), 4.58 (d, J=11.7 Hz, 1H). 4.00 (d, J=11.7 Hz, 1H), 3.73-3.80 (m, 1H), 1.85-2.04 (m, 9H), 1.56-1.67 (m, 2H). LC-MS: m/z 438 [M+H]⁺.

### Method X1

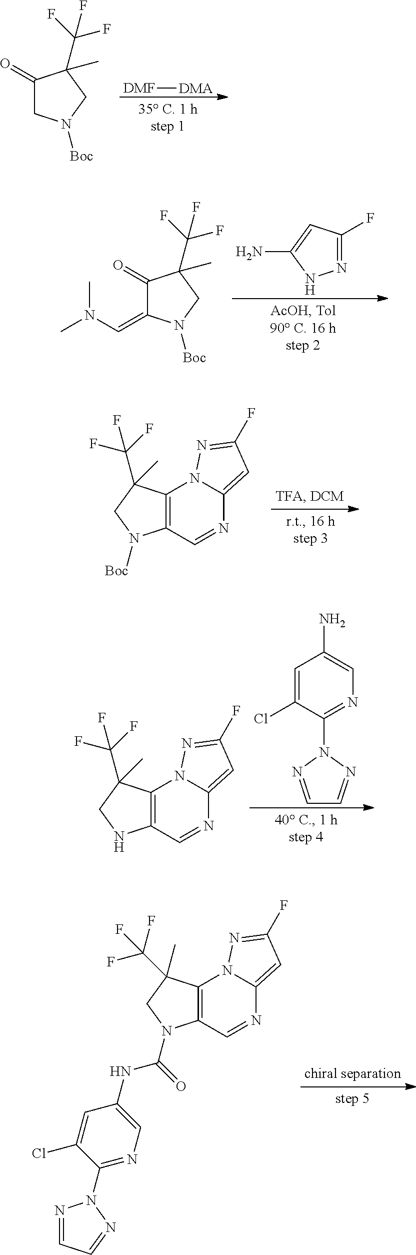

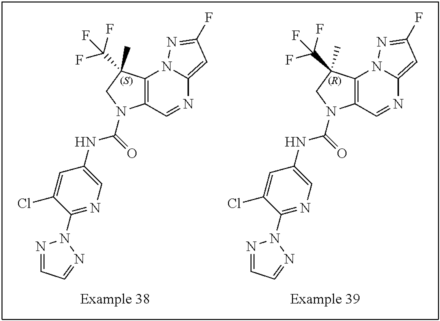

### Example 38: (S)—N-(5-chloro-6-(2H-1,2,3-triazol-2-yl)pyridin-3-yl)-2-fluoro-8-methyl-8 (trifluoromethyl)-7,8-dihydro-6H-pyrazolo[1,5-a]pyrrolo[2,3-]pyrimidine-6-carboxamide

### Example 39: (R)—N-(5-chloro-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

### Step 1: tert-butyl 2-((dimethylamino)methylene)-4-methyl-3-oxo-4-(trifluoromethyl)pyrrolidine-1-carboxylate

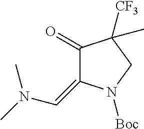

A mixture of tert-butyl 3-methyl-4-oxo-3-(trifluoromethyl)pyrrolidine-1-carboxylate (500 mg, 1.9 mmol) and 1,1-dimethoxy-N,N-dimethylmethanamine (8.9 g, 74.7 mmol) was stirred at 35° C. for 1 h. The reaction mixture was concentrated under reduced pressure to give the crude tert-butyl 2-((dimethylamino)methylene)-4-methyl-3-oxo-4-(trifluoromethyl)pyrrolidine-1-carboxylate (500 mg, 74% yield) as a yellow oil. LC-MS: m/z 323 [M+H]⁺.

### Step 2: tert-butyl 2-fluoro-8-methyl-8-(trifluoromethyl)-7,8-dihydro-6H-pyrazolo[1,5-a]pyrrolo[2,3-e]pyrimidine-6-carboxylate

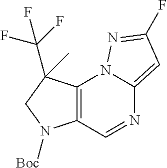 [SMILES: CC(C)(C)OC(=O)N1CC(C)(C(F)(F)F)c2c1cnc1cc(F)nn21]

To a stirred solution of tert-butyl 2-((dimethylamino)methylene)-4-methyl-3-oxo-4-(trifluoromethyl)pyrrolidine-1-carboxylate (526 mg, 1.6 mmol) and 3-fluoro-1H-pyrazol-5-amine (150 mg, 1.5 mmol) in toluene (2 mL) was added acetic acid (210 mg, 3.5 mmol). The resulting mixture was stirred for 16 h at 90° C. under nitrogen. The mixture was allowed to cool down to room temperature and concentrated under reduced pressure. The residue was diluted with water (10 mL). The pH was adjusted to 6-7 with sodium bicarbonate (sat., aq.) and the resulting mixture was extracted with EtOAc (2×10 mL). The combined organic layers were dried over anhydrous Na₂SO₄ and concentrated under vacuum. The residue was applied on a silica gel column chromatography and eluted with EtOAc/PE (1:5) to give tert-butyl 2-fluoro-8-methyl-8-(trifluoromethyl)-7,8-dihydro-6H-pyrazolo[1,5-a]pyrrolo[2,3-e]pyrimidine-6-carboxylate (90 mg, 16% yield) as a yellow solid. ¹H NMR (400 MHz, Chloroform-d) δ: 9.26 (s, 1H), 6.29 (d, J=5.2 Hz, 1H), 4.43 (d, J=10.8 Hz, 1H), 3.81 (d, J=10.8 Hz, 1H), 1.95 (s, 3H), 1.58 (s, 9H). LC-MS: m/z 361 [M+H]⁺.

### Step 3: 2-fluoro-8-methyl-8-(trifluoromethyl)-7,8-dihydro-6H-pyrazolo[1,5-a]pyrrolo[2,3-e]pyrimidine

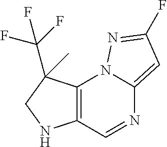 [SMILES: CC1(C(F)(F)F)CNc2cnc3cc(F)nn3c21]

To a stirred solution of tert-butyl 2-fluoro-8-methyl-8-(trifluoromethyl)-7,8-dihydro-6H-pyrazolo[1,5-a]pyrrolo[2,3-e]pyrimidine-6-carboxylate (80 mg, 222 μmol) in dichloromethane (0.5 mL) was added 2,2,2-trifluoroacetic acid (740 mg, 6.5 mmol). The reaction was stirred at room temperature for 1.5 h under nitrogen. The pH was adjusted to 6-7 with sodium bicarbonate (sat., aq.). The resulting solution was extracted with DCM (2×5 mL). The combined organic layers were dried over anhydrous Na₂SO₄ and concentrated under vacuum to give 2-fluoro-8-methyl-8-(trifluoromethyl)-7,8-dihydro-6H-pyrazolo[1,5-a]pyrrolo[2,3-e]pyrimidine (50 mg, 85% yield) as a yellow oil. ¹H NMR (300 MHz, Chloroform-d) δ: 8.27 (s, 1H), 6.23 (d, J=5.1 Hz, 1H), 4.08 (d, J=11.4 Hz, 1H), 3.56 (dd, J=11.4, 1.2 Hz, 1H), 1.89 (s, 3H). LC-MS: m/z 261 [M+H]⁺.

### Step 4: N-(5-chloro-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

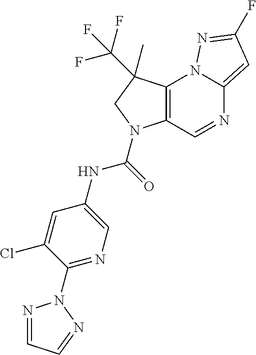 [SMILES: CC1(C(F)(F)F)CN(C(=O)Nc2cnc(-n3nccn3)c(Cl)c2)c2cnc3cc(F)nn3c21]

To a stirred mixture of 5-chloro-6-(2H-1,2,3-triazol-2-yl)pyridin-3-amine (Method A1 step 2; 101 mg, 518 μmol) and triphosgene (77 mg, 259 μmol) in tetrahydrofuran (2 mL) was added TEA (52 mg, 519 μmol). The reaction mixture was stirred at 35° C. for 1 h. The resulting mixture was filtered, and the filtrate was added to a stirred mixture of 2-fluoro-8-methyl-8-(trifluoromethyl)-7,8-dihydro-6H-pyrazolo[1,5-a]pyrrolo[2,3-e]pyrimidine (45 mg, 173 μmol), TEA (262 mg, 2.6 mmol) and N,N-dimethylpyridin-4-amine (42 mg, 346 μmol) in THF (2 mL). The reaction mixture was stirred at 40° C. for 1 h. The solvent was concentrated under vacuum. The residue was applied on a silica gel column chromatography and eluted with MeOH/DCM (1:10) to give N-(5-chloro-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide (80 mg, 95% yield) as an off-white solid. LC-MS: m/z 482 [M+H]⁺.

### Step 5: Separation of enantiomers to obtain (S)—N-(5-chloro-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide and (R)—N-(5-chloro-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

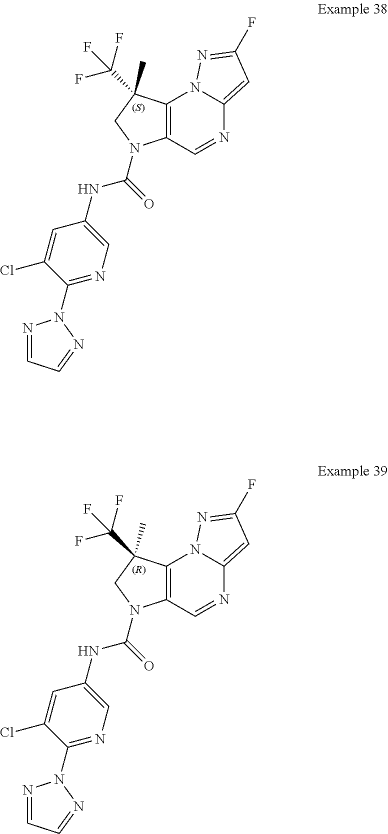

80 mg of N-(5-chloro-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide was submitted to chiral HPLC purification (CHIRALPAK IA, 2×25 cm, 5 um; Mobile Phase A: Hex (0.5% 2M NH₃-MeOH)—HPLC, Mobile Phase B: EtOH—HPLC; Flow rate: 20 mL/min; isocratic 20% B; 220/254 nm; RT1: 8.496; RT2: 10.912; Injection Volume: 0.5 ml; Number of Runs: 7). The first eluting isomer (RT 8.50 min) was concentrated and lyophilized to afford Example 38 as an off-white solid (13.6 mg, 16% yield). The second eluting isomer (RT 10.91 min) was concentrated and lyophilized to afford Example 39 as an off-white solid (14.9 mg, 18% yield).

Example 38: 1H NMR (400 MHz, Chloroform-d) δ: 9.41 (s, 1H), 8.58 (s, 1H), 8.46 (s, 1H), 7.96 (s, 2H), 6.82 (s, 1H), 6.36 (d, J=5.2 Hz, 1H), 4.61 (d, J=10.0 Hz, 1H), 4.08 (d, J=10.0 Hz, 1H), 2.06 (s, 3H). LC-MS: m/z 482 [M+H]⁺.

Example 39: ¹H NMR (400 MHz, Chloroform-d) δ: 9.41 (s, 1H), 8.58 (s, 1H), 8.43 (s, 1H), 7.96 (s, 2H), 6.88 (s, 1H), 6.36 (d, J=4.8 Hz, 1H), 4.61 (d, J=10.0 Hz, 1H), 4.08 (d, J=9.6 Hz, 1H), 2.06 (s, 3H). LC-MS: m/z 482 [M+H]⁺.

### Method Y1

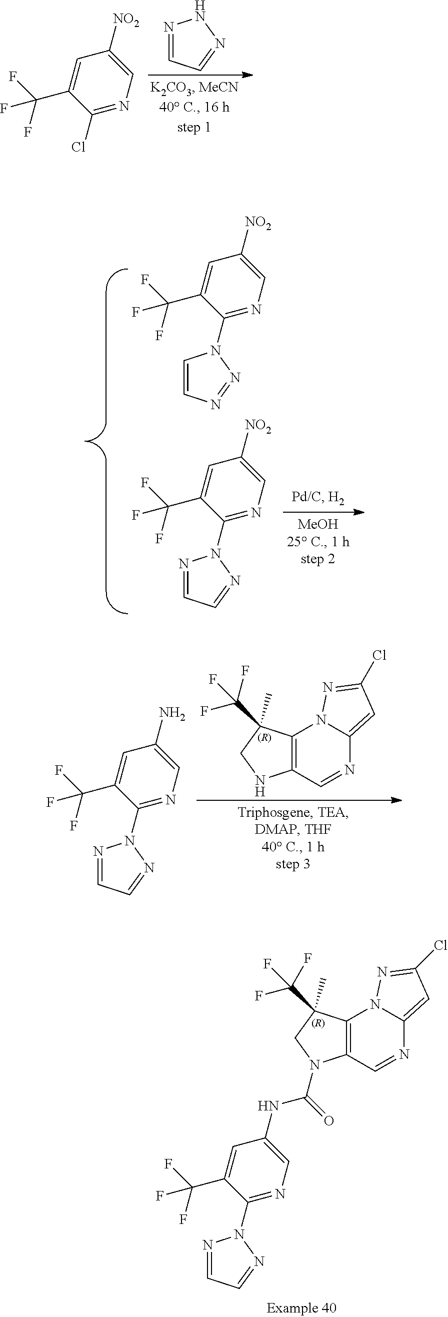

### Example 40: (R)—N-(6-(2H-1,2,3-triazol-2-yl)-5-(trifluoromethyl)pyridin-3-yl)-2-chloro-8-methyl-8-(trifluoromethyl)-7,8-dihydro-6H-pyrazolo[1,5-a]pyrrolo[2,3-e]pyrimidine-6-carboxamide

### Step 1: 5-nitro-2-(2H-1,2,3-triazol-2-yl)-3-(trifluoromethyl)pyridine and 5-nitro-2-(1H-1,2,3-triazol-1-yl)-3-(trifluoromethyl)pyridine

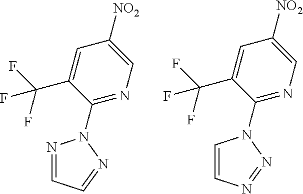

To a stirred solution of 2-chloro-5-nitro-3-(trifluoromethyl)pyridine (2 g, 8.8 mmol) in MeCN (40 mL) was added 2H-triazole (670 mg, 9.7 mmol) and K₂CO₃ (2.4 g, 51.8 mmol). The resulting mixture was stirred for 16 h at 40° C. The mixture was allowed to cool down to 25° C. The reaction mixture was filtered and the collected solid was washed with EtOAc (3×50 mL). The combined organic layers were concentrated under reduced pressure. The residue was applied on a silica gel column chromatography and eluted with EtOAc/PE (1:3) to give 5-nitro-2-(2H-1,2,3-triazol-2-yl)-3-(trifluoromethyl)pyridine (1.2 g, 52% yield) as a white solid and 5-nitro-2-(1H-1,2,3-triazol-1-yl)-3-(trifluoromethyl)pyridine (0.4 g, 17% yield) as a white solid. 5-nitro-2-(2H-1,2,3-triazol-2-yl)-3-(trifluoromethyl)pyridine ¹H NMR (300 MHz, DMSO-d₆) δ: 9.70 (d, J=4 Hz, 1H), 9.17 (d, J=4 Hz, 1H), 8.87 (s, 2H). LC-MS: m/z 260 [M+H]⁺. 5-nitro-2-(1H-1,2,3-triazol-1-yl)-3-(trifluoromethyl)pyridine ¹H NMR (300 MHz, DMSO-d₆) δ: 9.71 (d, J=3.6 Hz, 1H), 9.22 (d, J=3.2 Hz, 1H), 8.86 (d, J=1.6 Hz, 1H), 8.10 (d, J=1.6 Hz, 1H). LC-MS: m/z 260 [M+H]⁺.

### Step 2: 6-(2H-1,2,3-triazol-2-yl)-5-(trifluoromethyl)pyridin-3-amine

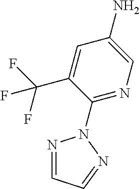 [SMILES: Nc1cnc(-n2nccn2)c(C(F)(F)F)c1]

To a solution of 5-nitro-2-(2H-1,2,3-triazol-2-yl)-3-(trifluoromethyl)pyridine (1.2 g, 4.4 mmol) was added Pd/C (10%, 236 mg) at 25° C. The flask was evacuated and flushed three times with nitrogen, followed by flushing with hydrogen. The mixture was stirred 1 h at room temperature under an atmosphere of hydrogen. The solid was filtered out. The filtrate was concentrated under reduced pressure. The residue was applied on a silica gel column chromatography and eluted with EtOAc/PE (1:1) to afford 6-(2H-1,2,3-triazol-2-yl)-5-(trifluoromethyl)pyridin-3-amin (800 mg, 78% yield) as yellow oil. LC-MS: m/z 230 [M+H]⁺.

### Step 3: (R)—N-(6-(2H-1,2,3-triazol-2-yl)-5-(trifluoromethyl)pyridin-3-yl)-2-chloro-8-methyl-8-(trifluoromethyl)-7,8-dihydro-6H-pyrazolo[1,5-a]pyrrolo[2,3-e]pyrimidine-6-carboxamide

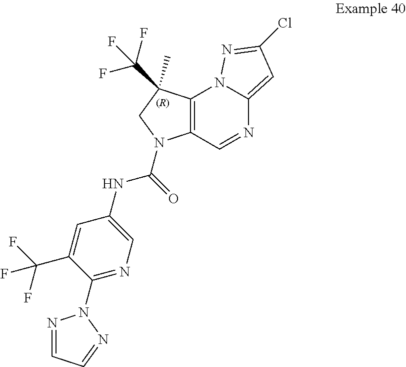

To a mixture of 6-(2H-1,2,3-triazol-2-yl)-5-(trifluoromethyl)pyridin-3-amine (32 mg, 135.6 μmol) in THF (5 mL) were added triphosgene (16 mg, 54.2 μmol) and TEA (12 mg, 135.6 μmol) at 25° C. The resulting mixture was stirred for 1 h at 28° C. and then filtered. The resulting filtrate was added to a solution of Method M1 isomer 2 (25 mg, 90.4 μmol) in THF (1 mL). To this solution was then added TEA (92 mg, 2.7 mmol) and N,N-Dimethylpyridin-4-amine (23 mg, 180.8 μmol). The reaction mixture was stirred for 1 h at 40° C. The residue was diluted with water (50 mL) and extracted with EtOAc (3×50 mL). The combined organic layers were washed with saturated aqueous ammonium chloride solution (3×50 mL), dried over anhydrous Na₂SO₄ and concentrated under vacuum. The residue was submitted to Prep-HPLC purification and the collected fractions were lyophilized to give (R)—N-(6-(2H-1,2,3-triazol-2-yl)-5-(trifluoromethyl) pyridin-3-yl)-2-chloro-8-methyl-8-(trifluoromethyl)-7,8-dihydro-6H-pyrazolo[1,5-a]pyrrolo[2,3-e]pyrimidine-6-carboxamide (18.1 mg, 54% yield) as a white solid. The enantiomer of Example 40 can be prepared analogously using Method M1 isomer 1.

Example 40: ¹H NMR (400 MHz, DMSO-d₆) δ: 9.86 (s, 1H), 9.37 (s, 1H), 9.08 (d, J=2 Hz, 1H), 8.72 (d, J=2.4 Hz, 1H), 8.20 (s, 2H), 7.09 (s, 1H), 4.86-4.89 (m, 1H), 4.31-4.34 (m, 1H), 2.00 (s, 3H). LC-MS: m/z 532 [M+H]⁺.

### Example 41: (R)-2-chloro-N-(5-cyano-6-(2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

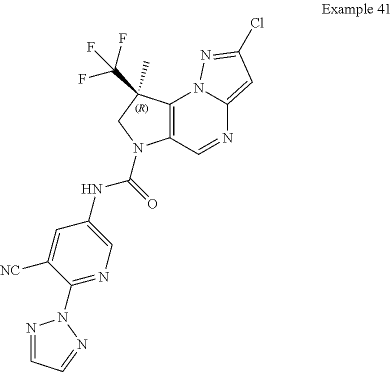

The title compound was prepared according to Method O1 step 3 by using 5-amino-2-(2H-1,2,3-triazol-2-yl)nicotinonitrile and Method M1 isomer 2. The enantiomer of Example 41 can be prepared analogously using Method M1 isomer 1.

Example 41: ¹H NMR (300 MHz, DMSO-d₆) δ: 9.82 (s, 1H), 9.39 (s, 1H), 8.96 (s, 1H), 8.72 (s, 1H), 8.29 (s, 2H), 7.07 (s, 1H), 4.83 (d, J=11.6 Hz, 1H), 4.29 (d, J=11.6 Hz, 1H), 1.99 (s, 3H). LC-MS: m/z 489 [M+H]⁺.

### Example 42: (R)-2-chloro-N-(5-chloro-6-cyanopyridin-3-yl)-8-methyl-8-(trifluoromethyl)-7,8-dihydro-6H-pyrazolo[1,5-a]pyrrolo[2,3-e]pyrimidine-6-carboxamide

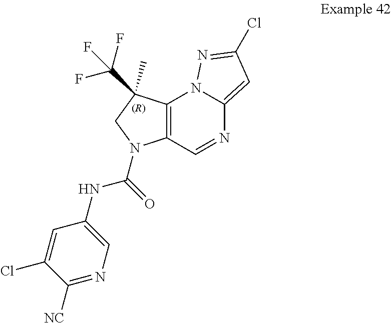

To a stirred mixture of 5-amino-3-chloro-pyridine-2-carbonitrile (20 mg, 130 μmol) in THF (4 mL) were added triphosgene (19 mg, 65 μmol) and TEA (16 mg, 162 μmol) at 25° C. The resulting mixture was stirred for 1 h at 25° C. and then filtered. The resulting filtrate was added to a solution of Method M1 isomer 2 (30 mg, 108 μmol) in THF (4 mL). To this solution was then added TEA (110 mg, 1.1 mmol) and DMAP (26 mg, 217 μmol). The reaction mixture was stirred for 2 h at 60° C. To the mixture was added EtOAc (20 mL). The mixture was washed with brine (2×20 mL), dried over anhydrous Na₂SO₄ and concentrated under reduced pressure. The residue was submitted to Prep-HPLC purification and the collected fractions were lyophilized to give (R)-2-chloro-N-(5-chloro-6-cyanopyridin-3-yl)-8-methyl-8-(trifluoromethyl)-7,8-dihydro-6H-pyrazolo[1,5-a]pyrrolo[2,3-e]pyrimidine-6-carboxamide (9.1 mg, 18% yield) as a white solid. The enantiomer of Example 42 can be prepared analogously using Method M1 isomer 1.

Example 42: ¹H NMR (300 MHz, DMSO-d₆) δ: 9.93 (s, 1H), 9.33 (s, 1H), 8.85 (s, 1H), 8.45 (s, 1H), 7.09 (s, 1H), 4.85 (d, J=2.4 Hz, 1H), 4.35 (d, J=2.4 Hz, 1H), 1.97 (s, 3H); LC-MS: m/z 456 [M+H]⁺.

### Method Z1

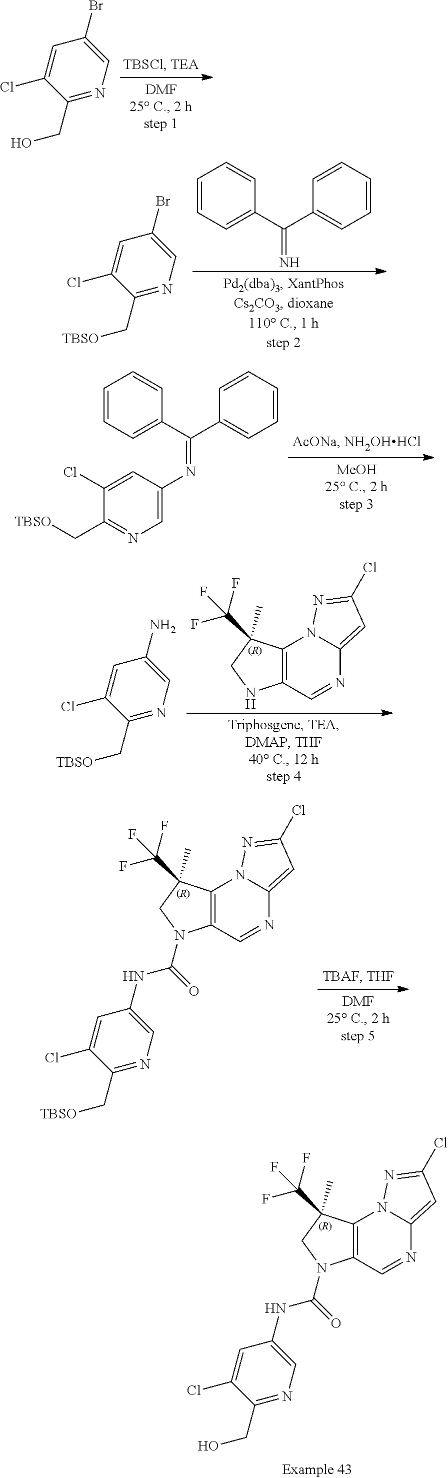

### Example 43: (R)-2-chloro-N-(5-chloro-6-(hydroxymethyl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 5-bromo-2-(((tert-butyldimethylsilyl)oxy)methyl)-3-chloropyridine

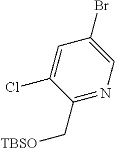 [SMILES: CC(C)(C)[Si](C)(C)OCc1ncc(Br)cc1Cl]

To a stirred solution of (5-bromo-3-chloropyridin-2-yl)methanol (500 mg, 2.25 mmol) and TEA (682.28 mg, 6.74 mmol) in DMF (5 mL) was added tert-butylchlorodimethylsilane (240.62 mg, 2.92 mmol) at 0° C. under nitrogen atmosphere. The resulting mixture was stirred for 2 h at 25° C. The LCMS showed the reaction was completed. The solution was poured into brine (10 mL) and the aqueous layer was extracted with EtOAc (3×10 mL). The combined organic layers were dried over anhydrous Na₂SO₄ and concentrated under reduced pressure. The residue was applied onto silica gel column chromatography and eluted with EtOAc/PE (1:2) to afford 5-bromo-2-(((tert-butyldimethylsilyl)oxy)methyl)-3-chloropyridine (580 mg, 77% yield) as a colorless oil. ¹HNMR (400 MHz, Chloroform-d) δ: 8.53 (d, J=2.0 Hz, 1H), 7.82 (d, J=2.0 Hz, 1H), 4.85 (d, J=6.4 Hz, 2H), 0.91 (s, 9H), 0.11 (s, 6H). LCMS (ES, m/z): 336[M+H]⁺.

### Step 2: N-(6-(((tert-butyldimethylsilyl)oxy)methyl)-5-chloropyridin-3-yl)-1,1-diphenylmethanimine

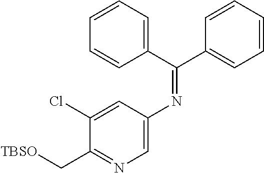 [SMILES: CC(C)(C)[Si](C)(C)OCc1ncc(N=C(c2ccccc2)c2ccccc2)cc1Cl]

To a stirred solution of 5-bromo-2-(((tert-butyldimethylsilyl)oxy)methyl)-3-chloropyridine (200 mg, 593.95 umol) and diphenylmethanimine (107.64 mg, 593.95 umol) in dioxane (5 ML) was added xantphos (103.10 mg, 178.19 μmol), Tris(dibenzylideneacetone)dipalladium-chloroform adduct (122.96 mg, 118.79 μmol) and Cs₂CO₃ (580.56 mg, 1.78 mmol) under nitrogen atmosphere. The resulting mixture was stirred for 2 h at 110° C. The reaction mixture was cooled to room temperature, and then poured into brine (10 mL). The aqueous layer was separated and further extracted with EtOAc (3×10 mL). The combined organic layers were dried over anhydrous Na₂SO₄, concentrated under reduced pressure. The residue was applied onto a silica gel column chromatography and eluting with EtOAc/PE (1:3) to afford N-(6-(((tert-butyldimethylsilyl)oxy)methyl)-5-chloropyridin-3-yl)-1,1-diphenylmethanimine (160 mg, 62% yield). ¹HNMR (400 MHz, Chloroform-d) δ: 7.73-7.86 (m, 3H), 7.53-7.59 (m, 1H), 7.41-7.49 (m, 2H), 7.28-7.36 (m, 3H), 7.10-7.23 (m, 3H), 0.87 (s, 9H), 0.03 (s, 6H). LCMS (ES, m/z): 437 [M+H]⁺.

### Step 3: 6-(((tert-butyldimethylsilyl)oxy)methyl)-5-chloropyridin-3-amine

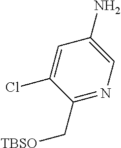 [SMILES: CC(C)(C)[Si](C)(C)OCc1ncc(N)cc1Cl]

To a stirred solution of N-(6-(((tert-butyldimethylsilyl)oxy)methyl)-5-chloropyridin-3-yl)-1,1-diphenylmethanimine (120 mg, 274.57 μmol) was added hydroxylamine hydrochloride (38.16 mg, 549.14 μmol), AcONa (93.41 mg, 686.42 μmol) and MeOH (3 mL). The resulting mixture was stirred for 2 h at 25° C. The solution was then poured into ice-water (10 mL), and the residue was separated and further extracted with EtOAc (3×10 mL). The combined organic layers were dried over anhydrous Na₂SO₄, concentrated under reduced pressure. The residue was applied onto a silica gel column chromatography and eluted with EtOAc/PE (1:4) to afford 6-(((tert-butyldimethylsilyl)oxy)methyl)-5-chloropyridin-3-amine (60 mg, 80% yield). ¹HNMR (400 MHz, Chloroform-d) δ: 8.03 (s, 1H), 7.04 (s, 1H), 4.81 (s, 2H), 0.91 (s, 9H), 0.11 (s, 6H). LCMS (ES, m/z): 273[M+H]⁺.

### Step 4: (R)—N-(6-(((tert-butyldimethylsilyl)oxy)methyl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide

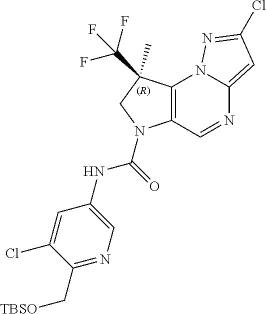

To a stirred mixture of 6-(((tert-butyldimethylsilyl)oxy)methyl)-5-chloropyridin-3-amine (29.59 mg, 108.44 μmol) in THF (4 mL) was added triphosgene (12.87 mg, 43.38 μmol) and TEA (10.97 mg, 108.44 μmol). The resulting mixture was stirred for 0.5 h at 25° C. and then filtered. To the filtrate was added a solution of Method M1 isomer 2 (20 mg, 72.29 umol), TEA (73.16 mg, 722.95 μmol) and N,N-dimethylpyridin-4-amine (17.66 mg, 144.59 μmol). The resulting mixture was stirred for 12 h at 40° C. The resulting mixture was concentrated under reduced pressure. The residue was purified by Prep-TLC with MeOH/DCM (1:30) to afford (R)—N-(6-(((tert-butyldimethylsilyl)oxy)methyl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide (20 mg, 40% yield) as a white solid. LCMS (ES, m/z): 575[M+H]⁺

### Step 5: (R)-2-chloro-N-(5-chloro-6-(hydroxymethyl)pyridin-3-yl)-8-methyl-8-(trifluoromethyl)-7,8-dihydro-6H-pyrazolo[1,5-a]pyrrolo[2,3-e]pyrimidine-6-carboxamide

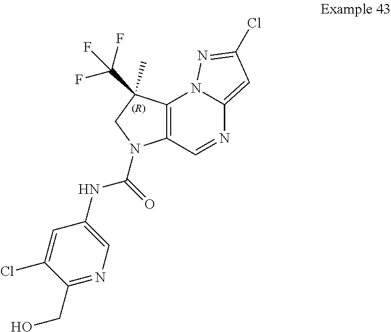

To a stirred solution of (R)—N-(6-(((tert-butyldimethylsilyl)oxy)methyl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide (18 mg, 31.28 μmol) in THF (10 mL) was added TBAF (1 mL, 3.45 mmol, 1 M in THF). The mixture was stirred for 2 h at 25° C. The reaction mixture was concentrated under reduced pressure. The residue was submitted to Prep-HPLC purification and the collected fractions were lyophilized to give (R)-2-chloro-N-(5-chloro-6-(hydroxymethyl)pyridin-3-yl)-8-methyl-8-(trifluoromethyl)-7,8-dihydro-6H-pyrazolo[1,5-a]pyrrolo[2,3-e]pyrimidine-6-carboxamide (6.2 mg, 43% yield) as an off-white solid. The enantiomer of Example 43 can be prepared analogously using Method M1 isomer 1.

Example 43: ¹HNMR (400 MHz, DMSO-d₆) δ: 9.48 (b, 1H), 9.34 (s, 1H), 8.67 (d, J=2.0 Hz, 1H), 8.17 (d, J=2.0 Hz, 1H), 7.07 (s, 1H), 5.15-5.30 (m, 1H), 4.75-4.85 (m, 1H), 4.60 (d, J=5.2 Hz, 2H), 4.30-4.23 (m, 1H), 1.97 (s, 3H). LCMS (ES, m/z): 461[M+H]⁺.

### Method A2

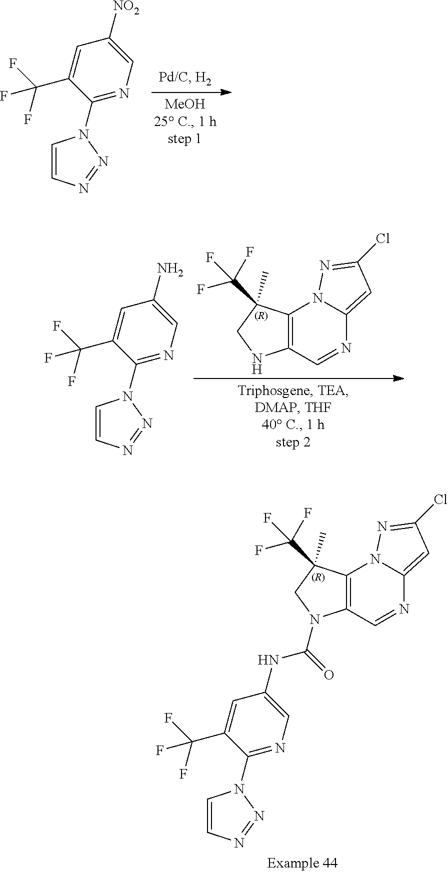

### Example 44: (R)—N-(6-(1H-1,2,3-triazol-1-yl)-5-(trifluoromethyl)pyridin-3-yl)-2-chloro-8-methyl-8-(trifluoromethyl)-7,8-dihydro-6H-pyrazolo[1,5-a]pyrrolo[2,3-e]pyrimidine-6-carboxamide

### Step 1: 6-(1H-1,2,3-triazol-1-yl)-5-(trifluoromethyl)pyridin-3-amine

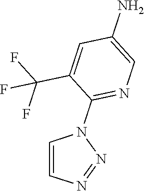 [SMILES: Nc1cnc(-n2ccnn2)c(C(F)(F)F)c1]

To a solution of 5-nitro-2-(1H-1,2,3-triazol-1-yl)-3-(trifluoromethyl)pyridine (Method Y1 step 1) (0.46 g, 1.78 mmol) was added Pd/C (10%, 95 mg) at 25° C. The flask was evacuated and flushed three times with nitrogen, followed by flushing with hydrogen. The mixture was stirred 1 h at room temperature under an atmosphere of hydrogen. The solid was filtered. The filtrate was concentrated under reduced pressure. The residue was applied onto a silica gel column chromatography and eluted with EtOAc/PE (1:1) to afford 6-(1H-1,2,3-triazol-1-yl)-5-(trifluoromethyl)pyridin-3-amine (300 mg, 73% yield) as a yellow oil. LC-MS: m/z 230 [M+H]⁺.

### Step 2: (R)—N-(6-(1H-1,2,3-triazol-1-yl)-5-(trifluoromethyl)pyridin-3-yl)-2-chloro-8-methyl-8-(trifluoromethyl)-7,8-dihydro-6H-pyrazolo[1,5-a]pyrrolo[2,3-e]pyrimidine-6-carboxamide

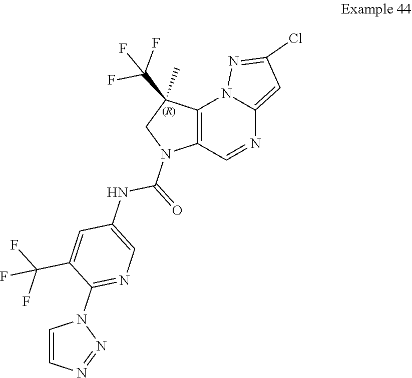

To a mixture of 6-(1H-1,2,3-triazol-1-yl)-5-(trifluoromethyl)pyridin-3-amine (37 mg, 126.6 μmol) in THF (6 mL) were added triphosgene (19 mg, 65.1 μmol) and TEA (17 mg, 163.1 μmol) at 25° C. The resulting mixture was stirred for 1 h at 28° C. and then filtered. The resulting filtrate was added to a solution of Method M1 isomer 2 (30 mg, 108.7 μmol) in THF (1 mL). To this solution was then added TEA (110 mg, 1.09 mmol) and N,N-Dimethylpyridin-4-amine (27 mg, 218.4 μmol). The reaction mixture was stirred for 1 h at 40° C. The reaction mixture was diluted with water (50 mL) and extracted with EtOAc (3×50 mL). The combined organic layers were washed with saturated aqueous ammonium chloride solution (3×50 mL). The resulting solution was dried over anhydrous Na₂SO₄ and concentrated under vacuum. The residue was submitted to Prep-HPLC purification and the collected fractions were lyophilized to give (R)—N-(6-(1H-1,2,3-triazol-1-yl)-5-(trifluoromethyl)pyridin-3-yl)-2-chloro-8-methyl-8-(trifluoromethyl)-7,8-dihydro-6H-pyrazolo[1,5-a]pyrrolo[2,3-e]pyrimidine-6-carboxamide (9.6 mg, 16% yield) as a white solid. The enantiomer of Example 44 can be prepared analogously using Method M1 isomer 1.

Example 44: ¹H NMR (400 MHz, DMSO-d₆) δ: 9.87 (s, 1H), 9.38 (s, 1H), 9.09 (s, 1H), 8.67-8.73 (m, 2H), 8.00 (s, 1H), 7.10 (s, 1H), 4.86-4.89 (m, 1H), 4.31-4.34 (m, 1H), 2.00 (s, 3H). LC-MS: m/z 532 [M+H]⁺.

### Method B2

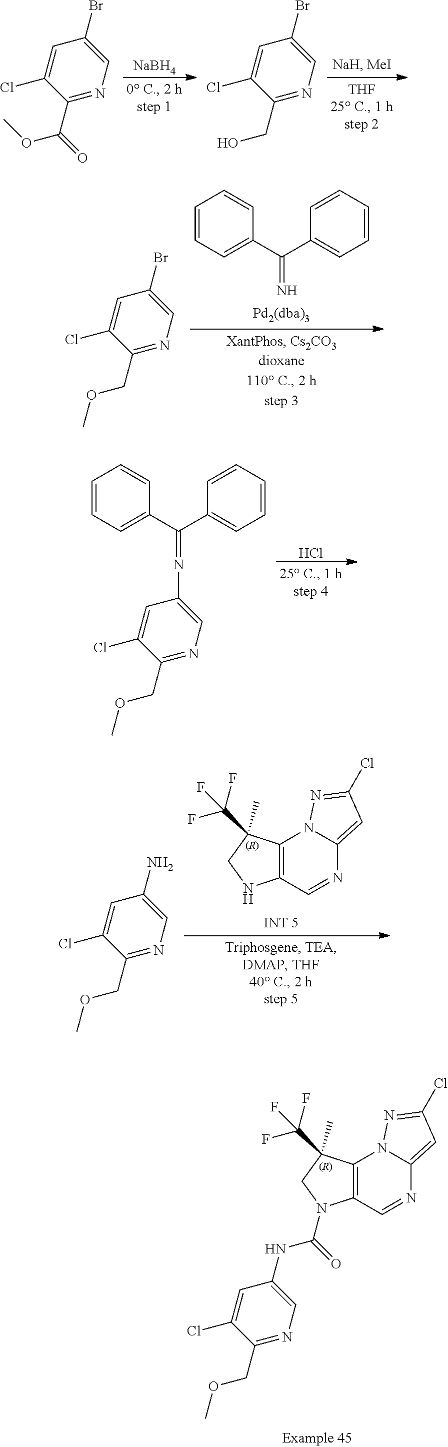

### Example 45: (R)-2-chloro-N-(5-chloro-6-(methoxymethyl)pyndin-3-yl)-8-methyl-8-(trifluoromethyl)-7,8-dihydro-6H-pyrazolo[1,5-a]pyrrolo[2,3-e]pyrimidine-6-carboxamide

### Step 1: (5-bromo-3-chloropyridin-2-yl)methanol

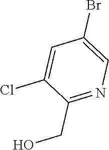

To a stirred solution of methyl 5-bromo-3-chloropicolinate (2.0 g, 8.0 mmol) in MeOH (30 mL) was added NaBH₄ (1.2 g, 32.0 mmol) at 0° C. The mixture was stirred at 0° C. for 2 h. The reaction was quenched by the addition of saturated aqueous NH₄Cl (20 mL) at 0° C. The resulting mixture was extracted with EtOAc (3×50 mL). The combined organic layers were washed with brine (80 mL) and dried over anhydrous Na₂SO₄. After filtration, the filtrate was concentrated under vacuum to afford (5-bromo-3-chloropyridin-2-yl)methanol (1.8 g, 81% yield) as a white solid. ¹H NMR (300 MHz, Chloroform-d) δ: 8.55 (d, J=2.1 Hz, 1H), 7.86 (d, J=2.1 Hz, 1H), 4.75 (s, 2H), 3.97 (s, 1H). LC-MS: m/z 222 [M+H]⁺.

### Step 2: 5-bromo-3-chloro-2-(methoxymethyl)pyridine

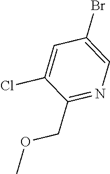

To a stirred solution of (5-bromo-3-chloropyridin-2-yl)methanol (1.0 g, 4.5 mmol) in THF (50 mL) was added NaH (60% in mineral oil, 216 mg, 5.4 mmol) in portions at 0° C. under nitrogen atmosphere. The mixture was stirred at 0° C. for 30 min. MeI (955 mg, 6.7 mmol) was added into the mixture. The mixture was stirred at 25° C. for 1 h. The reaction was quenched by the addition of water/ice (70 mL) at 0° C. The resulting mixture was extracted with EtOAc (3×50 mL). The combined organic layers were washed with brine (50 mL), dried over anhydrous Na₂SO₄, filtered and concentrated under vacuum. This resulted in 5-bromo-3-chloro-2-(methoxymethyl)pyridine (700 mg, 66% yield). ¹H NMR (400 MHz, Chloroform-d) δ: 8.58 (d, J=2.0 Hz, 1H), 7.86 (d, J=2.0 Hz, 1H), 4.64 (s, 2H), 3.49 (s, 3H). LC-MS: m/z 236 [M+H]⁺.

### Step 3: N-(5-chloro-6-(methoxymethyl)pyridin-3-yl)-1,1-diphenylmethanimine

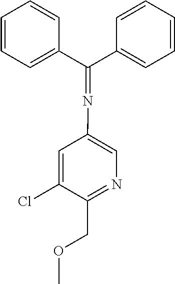 [SMILES: COCc1ncc(N=C(c2ccccc2)c2ccccc2)cc1Cl]

To a stirred solution of 5-bromo-3-chloro-2-(methoxymethyl)pyridine (300 mg, 1.3 mmol) and diphenylmethanimine (275 mg, 1.5 mmol) in dioxane (4 mL) were added XantPhos (220 mg, 380 μmol), Pd₂(dba)₃ (145 mg, 253 μmol) and Cs₂CO₃ (1.2 g, 3.8 mmol) under N₂. The mixture was stirred at 110° C. for 2 h. The mixture was allowed to cool down to room temperature. The resulting mixture was diluted with EtOAc (20 mL) and filtered. The filter cake was washed with EtOAc (3×20 mL). The filtrate was concentrated under vacuum. The residue was applied onto a silica gel column chromatography and eluted with EtOAc/PE (1:1) to afford N-[5-chloro-6-(methoxymethyl)-3-pyridyl]-1,1-diphenyl-methanimine (600 mg, 70% yield) as a yellow solid. ¹H NMR (400 MHz, Methanol-d₄) δ: 7.91 (d, J=2.4 Hz, 1H), 7.74 (d, J=2.4 Hz, 1H), 7.38-7.53 (m, 7H), 7.27-7.36 (m, 3H), 4.58 (s, 2H), 3.43 (s, 3H). LC-MS: m/z 337 [M+H]⁺.

### Step 4: 5-chloro-6-(methoxymethyl)pyridin-3-amine

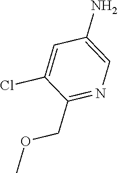

N-[5-chloro-6-(methoxymethyl)-3-pyridyl]-1,1-diphenyl-methanimine (600 mg, 1.9 mmol) was dissolved in HCl (4 mL, 12 N in H₂O). The mixture was stirred at 25° C. for 1 h. The resulting mixture was concentrated under vacuum. The residue was applied onto a silica gel column chromatography and eluted with MeOH/DCM (1:10) to afford 5-chloro-6-(methoxymethyl)pyridin-3-amine (110 mg, 35% yield) as a white solid. ¹H NMR (400 MHz, Chloroform-d) δ: 7.99 (d, J=2.4 Hz, 1H), 7.00 (d, J=2.4 Hz, 1H), 4.58 (s, 2H), 3.64-3.98 (m, 2H), 3.47 (s, 3H). LC-MS: m/z 173 [M+H]⁺.

### Step 5: (R)-2-chloro-N-(5-chloro-6-(methoxymethyl)pyridin-3-yl)-8-methyl-8-(trifluoromethyl)-7,8-dihydro-6H-pyrazolo[1,5-a]pyrrolo[2,3-e]pyrimidine-6-carboxamide

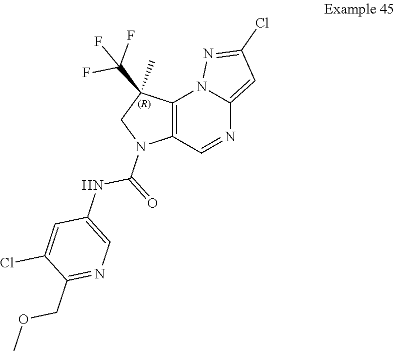

To a stirred solution of 5-chloro-6-(methoxymethyl)pyridin-3-amine (15 mg, 86 μmol) and triphosgene (13 mg, 43 μmol) in THF (4 mL) was added TEA (11 mg, 107 μmol) at 0° C. The resulting mixture was stirred for 0.5 h at 25° C. and then filtered. The resulting filtrate was added to a solution of Method M1 isomer 2 (20 mg, 72 μmol) in THF (1 mL). To this solution was then added TEA (73 mg, 722 μmol) and N,N-dimethylpyridin-4-amine (18 mg, 144 μmol). The mixture was stirred at 40° C. for 2 h. The mixture was poured into water (40 mL) and extracted with EtOAc (3×40 mL). The combined organic layers were washed with brine (50 mL), dried over anhydrous Na₂SO₄ and concentrated under vacuum. The residue was submitted to Prep-HPLC purification and the collected fractions were lyophilized to give (R)-2-chloro-N-(5-chloro-6-(methoxymethyl)pyridin-3-yl)-8-methyl-8-(trifluoromethyl)-7,8-dihydro-6H-pyrazolo[1,5-a]pyrrolo[2,3-e]pyrimidine-6-carboxamide (12.8 mg, 37% yield) as a white solid. The enantiomer of Example 45 can be prepared analogously using Method M1 isomer 1.

Example 45: ¹H NMR (300 MHz, DMSO-d₆) δ: 9.43 (s, 1H), 9.34 (s, 1H), 8.68 (d, J=2.1 Hz, 1H), 8.21 (d, J=2.1 Hz, 1H), 7.07 (s, 1H), 4.82 (d, J=11.4 Hz, 1H), 4.54 (s, 2H), 4.27 (d, J=11.4 Hz, 1H), 3.03-3.32 (m, 3H), 1.98 (s, 3H). LC-MS: m/z 475 [M+H]⁺.

### Method C2

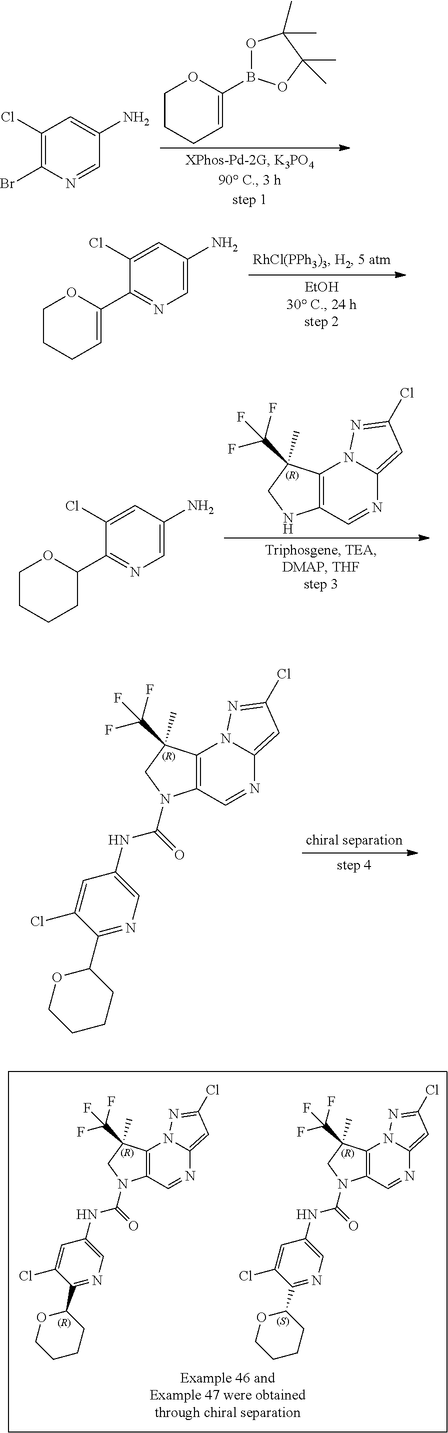

### Example 46 and Example 47: Single stereoisomers obtained from a mixture containing (R)-2-chloro-N-(5-chlor-6-((R)-tetrahydro-2H-pyran-2-yl)pyridin-3-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(5-chloro-6-((S)-tetrahydro-2H-pyran-2-yl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 5-chloro-6-(3,4-dihydro-2H-pyran-6-yl)pyridin-3-amine

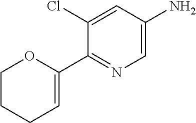 [SMILES: Nc1cnc(C2=CCCCO2)c(Cl)c1]

To a solution of 6-bromo-5-chloropyridin-3-amine (1 g, 4.8 mmol) in dioxane (16 mL) and H₂O (4 mL) were added 2-(3,4-dihydro-2H-pyran-6-yl)-4,4,5,5-tetramethyl-1,3,2-dioxaborolane (1.2 g, 5.8 mmol), K₃PO₄ (3.1 g, 14.5 mmol) and XPhos-Pd-2G (427 mg, 482.2 μmol). The resulting mixture was stirred for 3 h at 90° C. The mixture was allowed to cool down to room temperature and concentrated under vacuum. The residue was diluted with water (100 mL) and adjusted to pH 7-8 with NaHCO₃ (sat., aq.). The resulting mixture was extracted with EtOAc (3×100 mL). The combined organic layers were dried over anhydrous Na₂SO₄ and concentrated under vacuum. The residue was applied onto a silica gel column chromatography and eluted with EtOAc/PE (1:3) to give 5-chloro-6-(3,4-dihydro-2H-pyran-6-yl)pyridin-3-amine (380 mg, 37% yield) as a yellow solid. LC-MS: m/z 211 [M+H]⁺.

### Step 2: 5-chloro-6-(tetrahydro-2H-pyran-2-yl)pyridin-3-amine

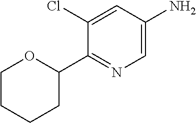 [SMILES: Nc1cnc(C2CCCCO2)c(Cl)c1]

To a solution of 5-chloro-6-(3,4-dihydro-2H-pyran-6-yl)pyridin-3-amine (385 mg, 1.8 mmol) in EtOH (5 mL) was added RhCl(PPh₃)₃ (485 mg, 541.5 μmol) under H₂ (5 atm). The resulting mixture was stirred for 24 h at 30° C. The reaction mixture was added water (100 mL). The resulting solution was extracted with EtOAc (3×100 mL). The combined organic layers were dried over anhydrous Na₂SO₄ and concentrated under vacuum. The residue was applied onto a silica gel column chromatography and eluted with EtOAc/PE (1:3) to give 5-chloro-6-(tetrahydro-2H-pyran-2-yl)pyridin-3-amine (150 mg, 39% yield) as a yellow solid. LC-MS: m/z 213 [M+H]⁺.

### Step 3: (8R)-2-chloro-N-(5-chloro-6-(tetrahydro-2H-pyran-2-yl)pyridin-3-yl)-8-methyl-8-(trifluoromethyl)-7,8-dihydro-6H-pyrazolo[1,5-a]pyrrolo[2,3-e]pyrimidine-6-carboxamide

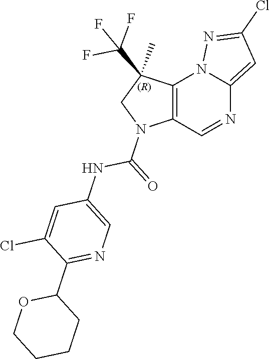

To a mixture of 5-chloro-6-(tetrahydro-2H-pyran-2-yl)pyridin-3-amine (30 mg, 141.1 μmol) in THF (2 mL) was added triphosgene (25 mg, 84.6 μmol) and TEA (21 mg, 211.5 μmol) at 25° C. The resulting mixture was stirred for 1 h at 25° C. and then filtered. The resulting filtrate was added to a solution of Method M1 isomer 2 (39 mg, 141.1 μmol) in THF (2 mL). To this solution was then added TEA (142 mg, 1.41 mmol) and N,N-Dimethylpyridin-4-amine (34 mg, 282.0 μmol). The reaction mixture was stirred for 1 h at 40° C. EtOAc (50 mL) was added to the reaction mixture and the organic layer was washed with brine (2×50 mL), dried over anhydrous Na₂SO₄ and concentrated. The residue was purified by Prep-TLC with MeOH/DCM(1:10) to afford (8R)-2-chloro-N-(5-chloro-6-(tetrahydro-2H-pyran-2-yl)pyridin-3-yl)-8-methyl-8-(trifluoromethyl)-7,8-dihydro-6H-pyrazolo[1,5-a]pyrrolo[2,3-e]pyrimidine-6-carboxamide (20 mg, 28% yield) as a light-yellow solid. ¹H NMR (400 MHz, DMSO-d₆) δ: 9.33 (s, 2H), 8.66 (d, J=2.4 Hz, 1H), 8.10 (d, J=2.4 Hz, 1H), 6.99 (s, 1H), 4.81 (d, J=11.6 Hz, 1H), 4.68-4.71 (m, 1H), 4.26 (d, J=11.6 Hz, 1H), 3.94 (s, 1H), 3.37 (s, 1H), 1.97 (s, 5H), 1.48-1.59 (m, 4H); LC-MS: m/z 515 [M+H]⁺.

### Step 4: Separation of stereoisomers to obtain (R)-2-chloro-N-(5-chloro-6-((R)-tetrahydro-2H-pyran-2-yl)pyridin-3-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(5-chloro-6-((S)-tetrahydro-2H-pyran-2-yl)pyridin-3-yl)-8-methyl-8-(trifluoromethyl)-7,8-dihydro-6H-pyrazolo[1,5-a]pyrrolo[2,3-e]pyrimidine-6-carboxamide

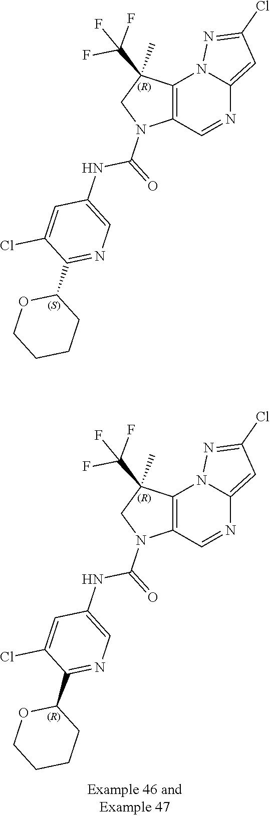

20 mg of (8R)-2-chloro-N-(5-chloro-6-(tetrahydro-2H-pyran-2-yl)pyridin-3-yl)-8-methyl-8-(trifluoromethyl)-7,8-dihydro-6H-pyrazolo[1,5-a]pyrrolo[2,3-e]pyrimidine-6-carboxamide was submitted to chiral HPLC purification (Column: CHIRAL ART Cellulose-SC, 2×25 cm, 5 um; Mobile Phase A: Hex (0.5% 2M NH₃-MeOH)—HPLC, Mobile Phase B: EtOH-HPLC; Flow rate: 20 mL/min; isocratic 50% B; 220/254 nm; RT1: 5.952; RT2: 7.605; Injection Volume: 1 ml; Number of Runs: 4). The first eluting isomer (RT 5.95 min) was concentrated and lyophilized to afford Example 46 as a white solid (8.0 mg, 29% yield). The second eluting isomer (RT 7.61 min) was concentrated and lyophilized to afford Example 47 as a white solid (8.5 mg, 32% yield). The corresponding enantiomers of Example 46 and Example 47 can be prepared analogously using Method M1 isomer 1.

Example 46: ¹H NMR (400 MHz, DMSO-d₆) δ: 9.33 (s, 2H), 8.67 (d, J=2.4 Hz, 1H), 8.10 (d, J=2.4 Hz, 1H), 6.99 (s, 1H), 4.76 (d, J=11.6 Hz, 1H), 4.63 (dd, J=11.6 Hz, 1.6 Hz, 1H), 4.18 (d, J=11.6 Hz, 1H), 3.94 (d, J=11.6 Hz, 1H), 3.45 (m, 1H), 1.97 (s, 5H), 1.48-1.59 (m, 4H); LC-MS: m/z 515 [M+H]⁺.

Example 47: ¹H NMR (400 MHz, DMSO-d₆) δ: 9.33 (s, 2H), 8.66 (d, J=2.4 Hz, 1H), 8.19 (d, J=2.4 Hz, 1H), 7.07 (s, 1H), 4.81 (d, J=11.6 Hz, 1H), (4.70 (dd, J=11.6 Hz, 1.6 Hz, 1H), 4.25 (d, J=11.6 Hz, 1H), 3.94 (d, J=11.6 Hz, 1H), 3.45 (m, 1H), 1.97 (s, 5H), 1.48-1.59 (m, 4H); LC-MS: m/z 515 [M+H]⁺.

### Method D2

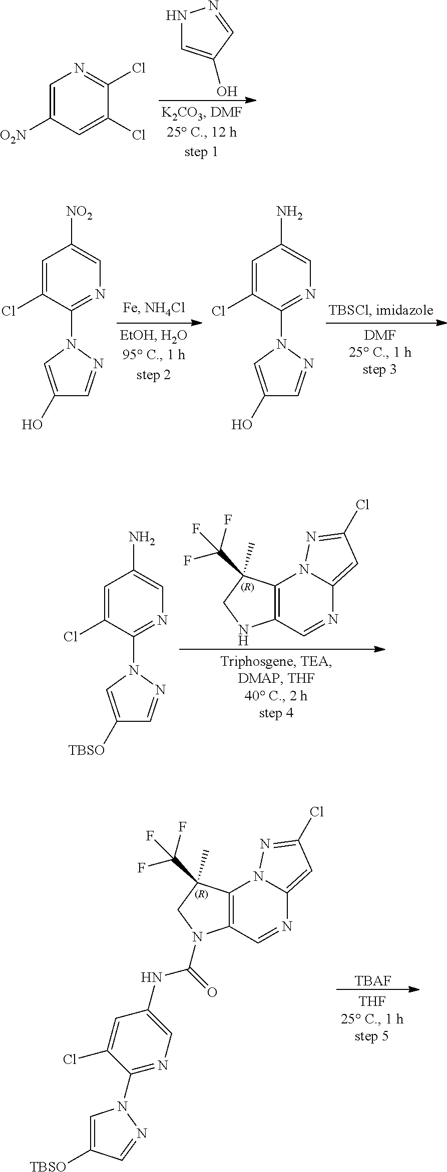

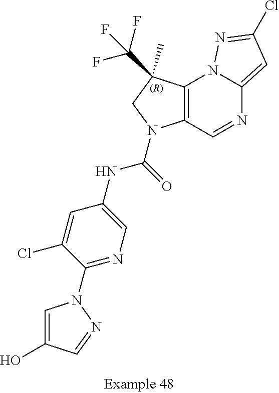

### Example 48: (R)—N-(6-(4-((tert-butyldimethylsilyl)oxy)-1H-pyrazol-1-yl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide

### Step 1: 1-(3-chloro-5-nitropyridin-2-yl)-1H-pyrazol-4-ol

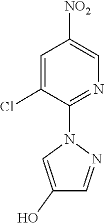 [SMILES: O=[N+]([O-])c1cnc(-n2cc(O)cn2)c(Cl)c1]

A solution of 2,3-dichloro-5-nitro-pyridine (1.5 g, 7.77 mmol), 1H-pyrazol-4-ol (653 mg, 7.8 mmol) and K₂CO₃ (3.2 g, 23.3 mmol) in DMF (30 mL) was stirred for 15 h at 25° C. The resulting mixture was poured into ice/water (200 mL), extracted with EtOAc (3×200 mL). The combined organic layers were washed with water (3×200 mL), brine (500 mL), dried over anhydrous Na₂SO₄, filtered and concentrated under reduced pressure. The residue was purified by silica column chromatography eluting with EtOAc/PE (3:7) to afford 1-(3-chloro-5-nitropyridin-2-yl)-1H-pyrazol-4-ol (1.5 g, 80% yield) as a yellow solid. ¹HNMR (300 MHz, DMSO-d₆) δ: 9.45 (s, 1H), 9.22 (d, J=4.0 Hz, 1H), 8.87 (d, J=4.0 Hz, 1H), 7.94 (d, J=4.0 Hz, 1H), 7.66 (s, 1H). LC-MS (ES, m/z): 241[M+H]⁺.

### Step 2: 1-(5-amino-3-chloropyridin-2-yl)-1H-pyrazol-4-ol

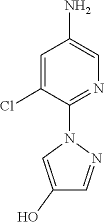 [SMILES: Nc1cnc(-n2cc(O)cn2)c(Cl)c1]

To a stirred solution of 1-(3-chloro-5-nitropyridin-2-yl)-1H-pyrazol-4-ol (700 mg, 2.9 mmol) in EtOH (30 mL) and H₂O (30 mL) were added iron (682 mg, 12.2 mmol) and ammonium chloride (654 mg, 12.2 mmol). The resulting mixture was stirred for 1 h at 95° C. The mixture was cooled down to room temperature. The reaction mixture was cooled and filtered, and the filtrate was concentrated under vacuum. The residue was purified with Prep-HPLC purification and the collected fractions were lyophilized to give 1-(5-amino-3-chloropyridin-2-yl)-1H-pyrazol-4-ol (410 mg, 67% yield) as an off-white solid. ¹HNMR (400 MHz, DMSO-d₆) δ: 8.70 (s, 1H), 7.65 (d, J=4.0 Hz, 1H), 7.42 (s, 1H), 7.27 (s, 1H), 7.14 (d, J=4.0 Hz, 1H), 5.88 (s, 2H). LC-MS (ES, m/z): 211 [M+H]⁺

### Step 3: 6-(4-((tert-butyldimethylsilyl)oxy)-1H-pyrazol-1-yl)-5-chloropyridin-3-amine

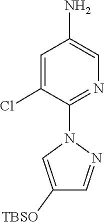 [SMILES: CC(C)(C)[Si](C)(C)Oc1cnn(-c2ncc(N)cc2Cl)c1]

To a stirred solution of 1-(5-amino-3-chloropyridin-2-yl)-1H-pyrazol-4-ol (150 mg, 712.2 μmol) and imidazole (73 mg, 1.1 mmol) in DMF (5 mL) was added TBSCl (129 mg, 854.6 μmol) dropwise at 0° C. under nitrogen atmosphere. The resulting mixture was stirred for 2 h. The LCMS showed the reaction was completed. The solution was poured into ice-water (10 mL) and the resulting mixture was extracted with EtOAc (3×10 mL). The combined organic layers were dried over anhydrous Na₂SO₄ and concentrated under reduced pressure. The residue was purified by silica gel column chromatography eluting with EtOAc/PE (1:4) to afford 6-(4-((tert-butyldimethylsilyl)oxy)-1H-pyrazol-1-yl)-5-chloropyridin-3-amine (200 mg, 83% yield) as a yellow oil. ¹HNMR (400 MHz, Chloroform-d) δ: 7.87 (d, J=2.8 Hz, 1H), 7.52 (d, J=0.8 Hz, 1H), 7.41 (d, J=0.8 Hz, 1H), 7.14 (d, J=2.8 Hz, 1H), 0.98 (s, 9H), 0.19 (s, 6H). LC-MS (ES, m/z): 325[M+H]⁺.

### Step 4: (R)—N-(6-(4-((tert-butyldimethylsilyl)oxy)-1H-pyrazol-1-yl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide

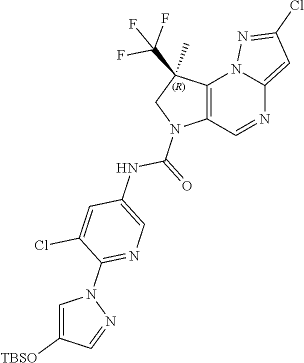

To a stirred mixture of 6-(4-((tert-butyldimethylsilyl)oxy)-1H-pyrazol-1-yl)-5-chloropyridin-3-amine (28 mg, 86.8 μmol) in THF (4 mL) was added triphosgene (13 mg, 43.4 μmol) and TEA (11 mg, 108.4 μmol). The mixture was stirred at 23° C. for 1 h. The resulting mixture was filtered, and the filtrate was added to a solution of Method M1 isomer 2 (20 mg, 72.3 μmol), N,N-dimethylpyridin-4-amine (18 mg, 144.6 μmol) and TEA (73 mg, 723.0 μmol, 101 μL) in THF (4 mL). The reaction mixture was stirred for 12 hours at 40° C. The reaction mixture was concentrated. The residue was purified by Prep-TLC with MeOH/DCM (1:30) to afford (R)—N-(6-(4-((tert-butyldimethylsilyl)oxy)-1H-pyrazol-1-yl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide (31 mg, 69% yield) as a white solid. LCMS (ES, m/z): 627[M+H]⁺.

Step 5: (R)-2-chloro-N-(5-chloro-6-(4-hydroxy-1H-pyrazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide

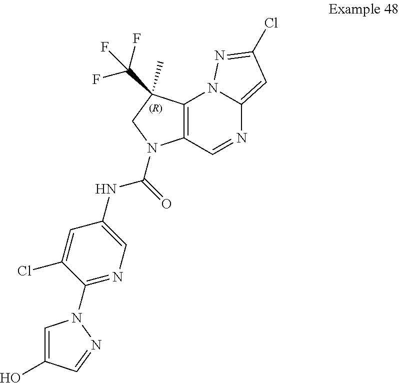

To a stirred mixture of (R)—N-(6-(4-((tert-butyldimethylsilyl)oxy)-1H-pyrazol-1-yl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide (30 mg, 47.8 μmol) in THF (3 mL) was added TBAF (0.3 mL, 1.0 mmol, 1 M in THF) dropwise at 25° C. The mixture was stirred for 1 h at the same temperature, and LCMS showed the reaction was complete. The mixture was concentrated under reduced pressure and the residue was purified by Prep-HPLC. Collected fractions were lyophilized to give (R)-2-chloro-N-(5-chloro-6-(4-hydroxy-1H-pyrazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide (12.1 mg, 48% yield) as a white solid. The corresponding enantiomer of Example 48 can be prepared analogously using Method M1 isomer 1.

Example 48: ¹HNMR (400 MHz, DMSO-d₆) δ: 9.52 (s, 1H), 9.35 (s, 1H), 8.96 (s, 1H), 8.61 (d, J=2.0 Hz, 1H), 8.34 (d, J=2.0 Hz, 1H), 7.68 (s, 1H), 7.41 (s, 1H), 7.07 (s, 1H), 4.81 (d, J=11.2 Hz, 1H), 4.26 (d, J=11.2 Hz, 1H), 1.98 (s, 3H). LCMS (ES, m/z): 513[M+H]⁺.

### Method E2

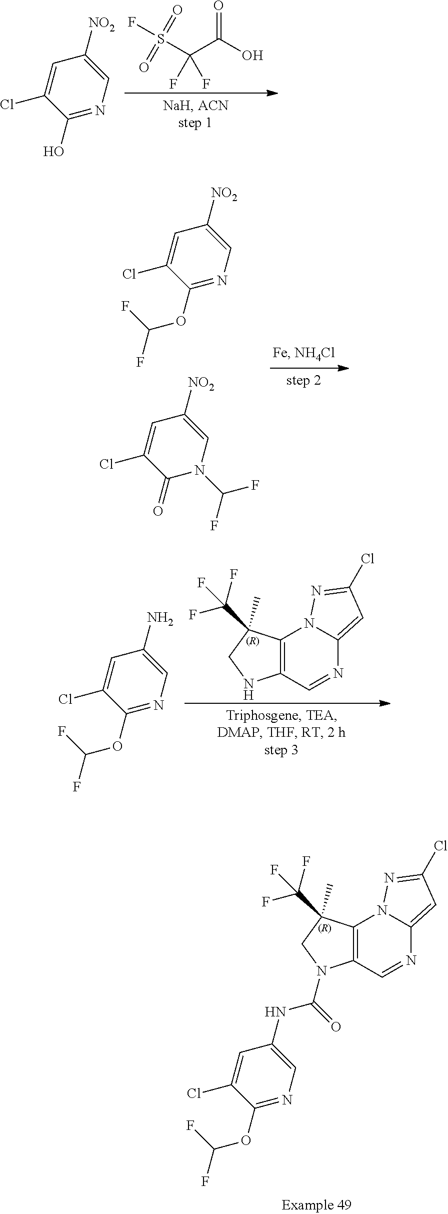

### Example 49: (R)-2-chloro-N-(5-chloro-6-(difluoromethoxy)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 3-chloro-2-(difluoromethoxy)-5-nitro-pyridine and 3-chloro-1-(difluoromethyl)-5-nitro-pyridin-2-one

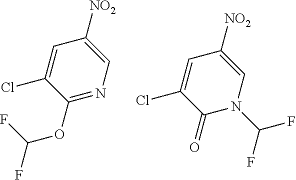 [SMILES: O=[N+]([O-])c1cnc(OC(F)F)c(Cl)c1 ; O=c1c(Cl)cc([N+](=O)[O-])cn1C(F)F]

To a stirred solution of 3-chloro-5-nitro-pyridin-2-ol (1 g, 5.7 mmol) in acetonitrile (50 mL) was added sodium hydride (618 mg, 15.4 mmol, 60% in mineral oil) at 0° C. The reaction mixture was stirred at 23° C. for 0.5 h. 2,2-difluoro-2-fluorosulfonyl-acetic acid (1.7 g, 9.7 mmol) was added and the mixture was stirred at 23° C. for 18 h. The reaction was quenched by the addition of water (50 mL), and the mixture was extracted with EtOAc (3×50 mL). The combined organic layers were washed with brine, dried over anhydrous sodium sulfate and concentrated. The residue was purified by prep-TLC (Petroleum ether:EtOAc=6:1) to afford 3-chloro-2-(difluoromethoxy)-5-nitro-pyridine (260 mg, 18% yield) as a colorless oil and 3-chloro-1-(difluoromethyl)-5-nitro-pyridin-2-one (70 mg, 4% yield) as a colorless oil.

3-chloro-2-(difluoromethoxy)-5-nitro-pyridine: ¹H NMR (400 MHz, Chloroform-d) δ: 8.98 (d, J=2.4 Hz, 1H), 8.60 (d, J=2.4 Hz, 1H), 7.52 (t, J=71.2 Hz, 1H).

3-chloro-1-(difluoromethyl)-5-nitro-pyridin-2-one: ¹H NMR (400 MHz, Chloroform-d) δ: 8.71 (1H, d, J=2.4 Hz), 8.36 (1H, d, J=2.8 Hz), 7.69 (1H, t, J=59.6 Hz).

### Step 2: 5-chloro-6-(difluoromethoxy)pyridin-3-amine

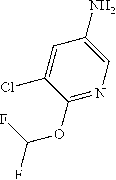 [SMILES: Nc1cnc(OC(F)F)c(Cl)c1]

To a mixture of 3-chloro-2-(difluoromethoxy)-5-nitro-pyridine (210 mg, 0.9 mmol) in ethanol (7.5 mL) and water (2.5 mL) was added ammonium chloride (100 mg, 1.9 mmol) and iron (313 mg, 5.6 mmol). The reaction mixture was stirred at 80° C. for 2 h. The reaction mixture was cooled and filtered, and the ethanol was removed under vacuum. The residue was extracted with EtOAc (3×10 mL), and the combined organic layers were washed with saturated aqueous ammonium chloride solution, dried over anhydrous sodium sulfate and concentrated under vacuum. The residue was applied onto a silica gel column and eluted with PE/EtOAc (3:1) to afford 5-chloro-6-(difluoromethoxy)pyridin-3-amine (140 mg, 50% yield) as a colorless oil. LC-MS: m/z 195 [M+H]⁺.

### Step 3: (R)-2-chloro-N-(5-chloro-6-(difluoromethoxy)pyridin-3-yl)-8-methyl-8-(trifluoromethyl)-7,8-dihydro-6H-pyrazolo[1,5-a]pyrrolo[2,3-e]pyrimidine-6-carboxamide

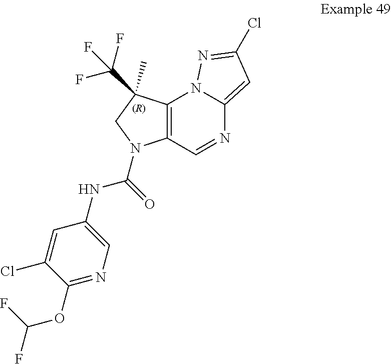

5-chloro-6-(difluoromethoxy)pyridin-3-amine (20 mg, 0.1 mmol) was added to a solution of bis(trichloromethyl) carbonate (15 mg, 0.05 mmol) and N,N-diethylethanamine (17 mg, 0.2 mmol) in tetrahydrofuran (2 mL). The mixture was stirred at 23° C. for 1 h. The resulting mixture was filtered, and the filtrate was added to a solution of Method M1 isomer 2 (23 mg, 0.1 mmol), N,N-diethylethanamine (86 mg, 0.8 mmol) and N,N-dimethylpyridin-4-amine (21 mg, 0.2 mmol) in tetrahydrofuran (2 mL). The resulting mixture was purified by Prep-HPLC and the collected fractions were lyophilized to give (R)-2-chloro-N-(5-chloro-6-(difluoromethoxy)pyridin-3-yl)-8-methyl-8-(trifluoromethyl)-7,8-dihydro-6H-pyrazolo[1,5-a]pyrrolo[2,3-e]pyrimidine-6-carboxamide (11 mg, 26% yield) as a white solid. The corresponding enantiomer of Example 49 can be prepared analogously using Method M1 isomer 1.

Example 49: ¹H NMR (400 MHz, DMSO-d₆) δ: 9.31 (s, 1H), 9.03 (s, 1H), 8.09 (d, J=2.8 Hz, 1H), 8.03 (d, J=2.4 Hz, 1H), 7.95 (t, J=59.6 Hz, 1H), 7.06 (s, 1H), 4.70 (d, J=11.6 Hz, 1H), 4.19 (d, J=11.2 Hz, 1H), 1.97 (s, 3H). LC-MS: m/z 497[M+H]⁺.

### Method F2

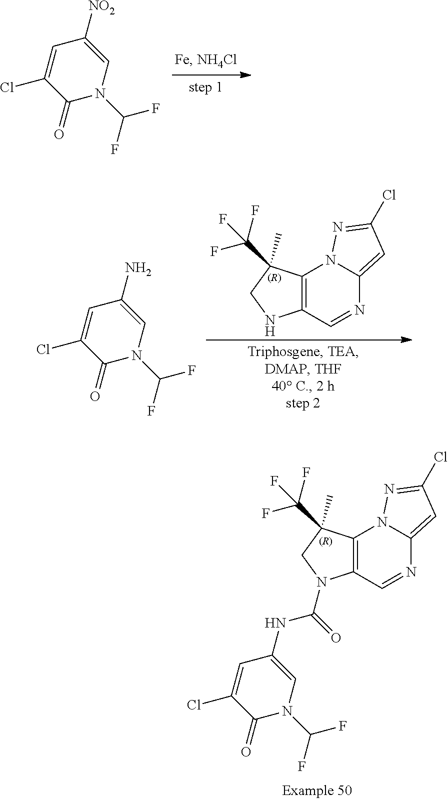

### Example 50: (R)-2-chloro-N-(5-chloro-1-(difluoromethyl)-6-oxo-1,6-dihydropyridin-3-yl)-8-methyl-8-(trifluoromethyl)-7,8-dihydro-6H-pyrazolo[1,5-a]pyrrolo[2,3-e]pyrimidine-6-carboxamide

### Step 1: 5-amino-3-chloro-1-(difluoromethyl)pyridin-2(1H)-one

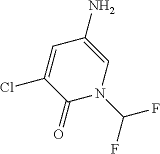 [SMILES: Nc1cc(Cl)c(=O)n(C(F)F)c1]

To a solution of 3-chloro-1-(difluoromethyl)-5-nitropyridin-2(1H)-one (Method E2 step 1; 70 mg, 0.3 mmol) in ethanol (1.5 mL) and water (0.5 mL) was added ammonium chloride (33 mg, 0.6 mmol) and iron (104 mg, 1.9 mmol). The reaction mixture was stirred at 80° C. for 2 h. The reaction mixture was cooled and filtered, and the filtrate was concentrated under vacuum. The residue was extracted with EtOAc (3×10 mL), and the combined organic layers were dried over anhydrous Na₂SO₄ and concentrated under vacuum. The residue was applied onto a silica gel column and eluted with EtOAc/PE (1:3) to afford 5-amino-3-chloro-1-(difluoromethyl)pyridin-2(1H)-one (20 mg, 26% yield) as a colorless oil. LC-MS: m/z 195 [M+H]⁺.

### Step 2: (R)-2-chloro-N-(5-chloro-1-(difluoromethyl)-6-oxo-1,6-dihydropyridin-3-yl)-8-methyl-8-(trifluoromethyl)-7,8-dihydro-6H-pyrazolo[1,5-a]pyrrolo[2,3-e]pyrimidine-6-carboxamide

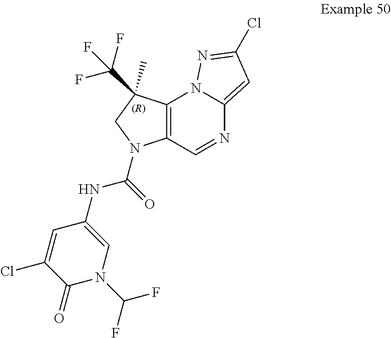

Method M1 isomer 2 (17 mg, 0.06 mmol) was added to a solution of triphosgene (11 mg, 0.03 mmol) and TEA (12 mg, 0.12 mmol) in THF (1 mL). The mixture was stirred at 23° C. for 1 h. The resulting mixture was filtered, and the filtrate was added to a solution of 5-amino-3-chloro-1-(difluoromethyl)pyridin-2(1H)-one (16 mg, 0.1 mmol), TEA (62 mg, 0.6 mmol) and N,N-dimethylpyridin-4-amine (15 mg, 0. mmol) in THF (1 mL). The reaction mixture was stirred at 40° C. for 2 h. The resulting mixture was purified with Prep-HPLC purification and the collected fractions were lyophilized to give (R)-2-chloro-N-(5-chloro-1-(difluoromethyl)-6-oxo-1,6-dihydropyridin-3-yl)-8-methyl-8-(trifluoromethyl)-7,8-dihydro-6H-pyrazolo[1,5-a]pyrrolo[2,3-e]pyrimidine-6-carboxamide (6.4 mg, 21% yield) as a light yellow solid. The corresponding enantiomer of Example 50 can be prepared analogously using Method M1 isomer 1.

Example 50: ¹H NMR (400 MHz, DMSO-d₆) δ: 9.39 (s, 1H), 9.33 (s, 1H), 8.37 (d, J=2.4 Hz, 1H), 8.32 (d, J=2.4 Hz, 1H), 7.71 (t, J=72.4 Hz, 1H), 7.07 (s, 1H), 4.78 (d, J=11.6 Hz, 1H), 4.25 (d, J=11.6 Hz, 1H), 1.98 (s, 3H). LC-MS: m/z 497 [M+H]⁺.

### Method G2

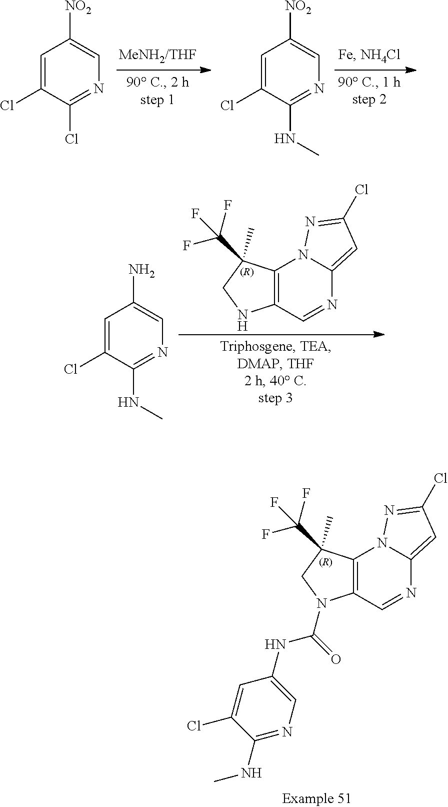

### Example 51: (R)-2-chloro-N-(5-chloro-6-(methylamino)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 3-chloro-N-methyl-5-nitropyridin-2-amine

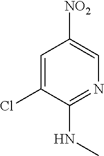

A solution of 2,3-dichloro-5-nitropyridine (500 mg, 2.6 mmol) in MeNH₂ (2 M in THF, 10 mL) was stirred for 2 h at 90° C. The mixture was filtrated. The filtrate was concentrated to afford 3-chloro-N-methyl-5-nitropyridin-2-amine (470 mg, 97% yield) as a yellow solid. ¹H NMR (300 MHz, Chloroform-d) δ: 9.02 (s, 1H), 8.28 (s, 1H), 5.79 (s, 1H), 3.17 (s, 3H); LC-MS: m/z 188 [M+H]⁺.

### Step 2: 3-chloro-N2-methylpyridine-2,5-diamine

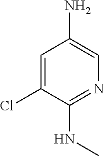

To a stirred solution of 3-chloro-N-methyl-5-nitro-pyridin-2-amine (470 mg, 2.5 mmol) in EtOH/H₂O (4:1, 10 mL) were added iron (279 mg, 5.0 mmol) and NH₄Cl (670 mg, 12.5 mmol). The resulting mixture was stirred for 1 h at 90° C. The mixture was filtered and the filtrate was diluted with water (50 mL) and extracted with EtOAc (3×50 mL). The combined organic layers were dried over anhydrous Na₂SO₄ and concentrated under vacuum to afford 3-chloro-N2-methylpyridine-2,5-diamine (160 mg, 40% yield) as a red oil. ¹H NMR (300 MHz, Chloroform-d) δ: 7.69 (s, 1H), 7.06 (s, 1H), 4.61 (s, 1H), 3.01 (s, 3H). LC-MS: m/z 158 [M+H]⁺.

### Step 3: (R)-2-chloro-N-(5-chloro-6-(methylamino)pyridin-3-yl)-8-methyl-8-(trifluoromethyl)-7,8-dihydro-6H-pyrazolo[1,5-a]pyrrolo[2,3-e]pyrimidine-6-carboxamide

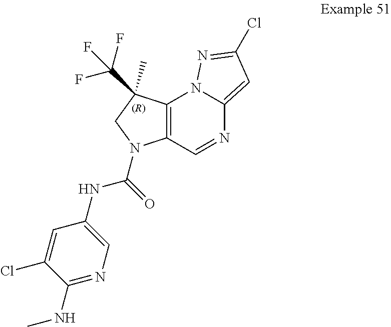

To a stirred mixture of Method M1 isomer 2 (20 mg, 74 μmol) in THF (2 mL) were added triphosgene (13 mg, 44 μmol) and TEA (11 mg, 111 μmol) at 25° C. The resulting mixture was stirred for 1 h at 25° C. and then filtered. The resulting filtrate was added to a mixture of 3-chloro-N2-methylpyridine-2,5-diamine (35 mg, 222 μmol) in THF (2 ML). TEA (75 mg, 740 μmol) and DMAP (18 mg, 148 μmol) were added and the reaction mixture was stirred for 2 h at 40° C. To the mixture was added EtOAc (50 mL) and the resulting organic layer was washed with brine (2×50 mL), dried and concentrated. The residue was submitted to Prep-HPLC purification and the collected fractions were lyophilized to give (R)-2-chloro-N-(5-chloro-6-(methylamino)pyridin-3-yl)-8-methyl-8-(trifluoromethyl)-7,8-dihydro-6H-pyrazolo[1,5-a]pyrrolo[2,3-e]pyrimidine-6-carboxamide (6.6 mg, 19.37% yield) as a white solid. The corresponding enantiomer of Example 51 can be prepared analogously using Method M1 isomer 1.

Example 51: ¹H NMR (300 MHz, Methanol-d₄) δ: 9.34 (s, 1H), 8.07 (s, 1H), 7.80 (s, 1H), 6.78 (s, 1H), 4.7 (d, J=2.4 Hz, 1H), 4.16 (d, J=2.4 Hz, 1H), 2.98 (s, 3H), 2.05 (s, 3H); LC-MS: m/z 460 [M+H]⁺.

### Method H2

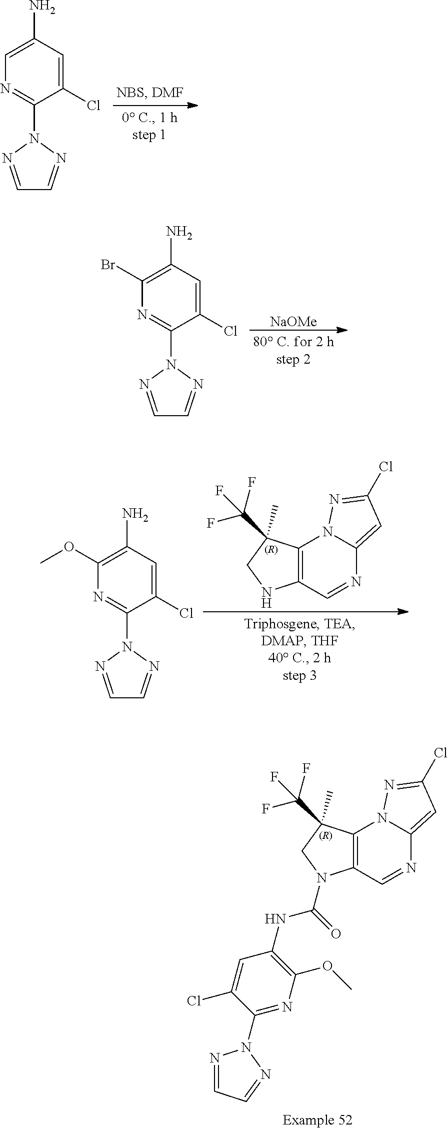

### Example 52: (R)-2-chloro-N-(5-chloro-2-methoxy-6-(2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 2-bromo-5-chloro-6-(2H-1,2,3-triazol-2-yl)pyridin-3-amine

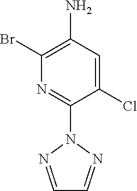 [SMILES: Nc1cc(Cl)c(-n2nccn2)nc1Br]

To a stirred solution of 5-chloro-6-(2H-1,2,3-triazol-2-yl)pyridin-3-amine (Method A1 step 2; 500 mg, 2.6 mmol) in DMF (10 mL) was slowly added NBS (682 mg, 3.8 mmol) in DMF (10 mL) at 0° C. The reaction mixture was stirred at 0° C. for 1 h. The resulting mixture was diluted with water (20 mL) and the mixture was extracted with EtOAc (3×20 mL). The combined organic layers were dried over anhydrous Na₂SO₄ and concentrated. The residue was applied onto a silica gel column and eluted with EtOAc/PE (1:2) to give 2-bromo-5-chloro-6-(2H-1,2,3-triazol-2-yl)pyridin-3-amine (560 mg, 72% yield) as a yellow solid. LC-MS: m/z 274 [M+H]⁺.

### Step 2: 5-chloro-2-methoxy-6-(2H-1,2,3-triazol-2-yl)pyridin-3-amine

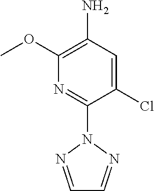

To a stirred solution of 2-bromo-5-chloro-6-(2H-1,2,3-triazol-2-yl)pyridin-3-amine (500 mg, 1.8 mmol) in dioxane (10 mL) was slowly added sodium methoxide (394 mg, 7.3 mmol) in MeOH (0.5 mL). The reaction mixture was stirred at 80° C. for 2 h. The mixture was concentrated under reduced pressure, and the residue was applied onto a silica gel column and eluted with EtOAc/PE (1:2) to give 5-chloro-2-methoxy-6-(2H-1,2,3-triazol-2-yl)pyridin-3-amine (350 mg, 71% yield) as a yellow solid. LC-MS: m/z 226 [M+H]⁺.

### Step 3: (R)-2-chloro-N-(5-chloro-2-methoxy-6-(2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

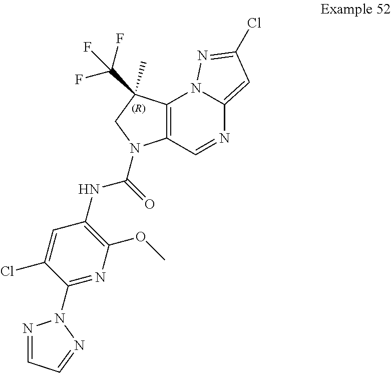

The title compound was prepared according to Method O1 step 3 by using 5-chloro-2-methoxy-6-(2H-1,2,3-triazol-2-yl)pyridin-3-amine and Method M1 isomer 2. The enantiomer of Example 52 can be prepared analogously using Method M1 isomer 1.

Example 52: 1H NMR (300 MHz, Chloroform-d) δ: 9.45 (s, 1H), 8.84 (s, 1H), 7.95 (s, 2H), 7.10 (s, 1H), 6.80 (s, 1H), 4.56 (d, J=10.2 Hz, 1H), 4.15 (s, 3H), 4.08 (d, J=10.2 Hz, 1H), 2.12 (s, 3H). LC-MS: m/z 528 [M+H]⁺.

### Method 12

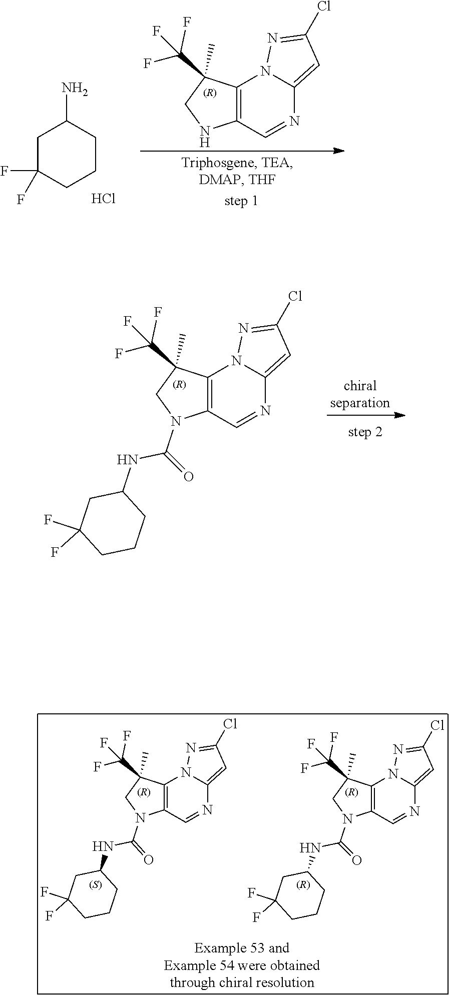

### Example 53 and 54: Single enantiomers obtained from a racemic mixture containing (R)-2-chloro-N—((S)-3,3-difluorocyclohexyl)-8-methyl-8-(trifluoromethyl)-7,8-dihydro-6H-pyrazolo[1,5-a]pyrrolo[2,3-e]pyrimidine-6-carboxamide and (R)-2-chloro-N—((R)-3,3-difluorocyclohexyl)-8-methyl-8-(trifluoromethyl)-7,8-dihydro-6H-pyrazolo[1,5-a]pyrrolo[2,3-e]pyrimidine-6-carboxamide

### Step 1: (8R)-2-chloro-N-(3,3-difluorocyclohexyl)-8-methyl-8-(trifluoromethyl)-7,8-dihydro-6H-pyrazolo[1,5-a]pyrrolo[2,3-e]pyrimidine-6-carboxamide

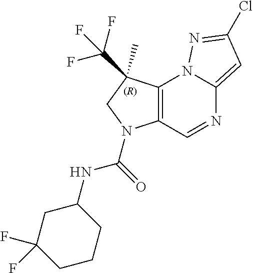

To a stirred solution of Method M1 isomer 2 (20 mg, 72.3 μmol) and bis(trichloromethyl) carbonate (13 mg, 43.4 μmol) in THF (4 mL) was added N,N-diethylethanamine (11 mg, 108.4 μmol, 15.1 μL) at 0° C. The mixture was stirred at 28° C. for 0.5 hr. The resulting mixture was added to a solution of 3,3-difluorocyclohexanamine hydrochloride salt (15 mg, 86.7 μmol) in THF (1 mL). To this solution was then added N,N-diethylethanamine (73 mg, 722.9 μmol, 100.7 μL) and N,N-dimethylpyridin-4-amine (18 mg, 144.6 μmol). The mixture was stirred at 28° C. for 2 h. The resulting mixture was purified with Prep-HPLC purification and the collected fractions were lyophilized to give 20 mg of (8R)-2-chloro-N-(3,3-difluorocyclohexyl)-8-methyl-8-(trifluoromethyl)-7,8-dihydro-6H-pyrazolo[1,5-a]pyrrolo[2,3-e]pyrimidine-6-carboxamide. LC-MS: m/z 438 [M+H]⁺.

### Step 2: Separation of enantiomers to obtain (R)-2-chloro-N—((S)-3,3-difluorocyclohexyl)-8-methyl-8-(trifluoromethyl)-7,8-dihydro-6H-pyrazolo[1,5-a]pyrrolo[2,3-e]pyrimidine-6-carboxamide and (R)-2-chloro-N—((R)-3,3-difluorocyclohexyl)-8-methyl-8-(trifluoromethyl)-7,8-dihydro-6H-pyrazolo[1,5-a]pyrrolo[2,3-e]pyrimidine-6-carboxamide

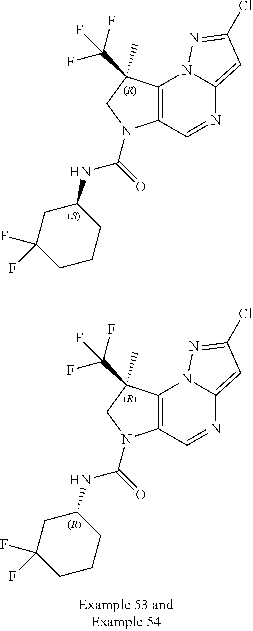

20 mg of (8R)-2-chloro-N-(3,3-difluorocyclohexyl)-8-methyl-8-(trifluoromethyl)-7,8-dihydro-6H-pyrazolo[1,5-a]pyrrolo[2,3-e]pyrimidine-6-carboxamide were submitted to chiral HPLC purification (CHIRALPAK IF, 2×25 cm, 5 um; Mobile Phase A: Hex (0.5% 2M NH₃-MeOH)—HPLC, Mobile Phase B: EtOH-HPLC; Flow rate: 20 mL/min; isocratic 5% B in 27 min; 220/254 nm; RTL: 18.869; RT2: 23.747; Injection Volume: 0.5 ml; Number of Runs: 9). The first eluting isomer (RT 18.87 min) was concentrated and lyophilized to afford Example 53 (11.8 mg, 37% yield). The second eluting isomer (RT 23.75 min) was concentrated and lyophilized to afford Example 54 (5.9 mg, 18% yield) as a white solid.

Example 53: ¹H NMR (300 MHz, DMSO-d₆) δ: 9.30 (s, 1H), 7.07 (d, J=7.5 Hz, 1H), 7.01 (s, 1H), 4.54 (d, J=11.4 Hz, 1H), 4.01 (d, J=11.4 Hz, 1H), 3.76-3.79 (m, 1H), 2.21-2.31 (m, 1H), 2.00-2.08 (m, 1H), 1.93 (s, 3H), 1.76-1.88 (m, 4H), 1.37-1.46 (m, 2H). LC-MS: m/z 438 [M+H]⁺.

Example 54: ¹H NMR (300 MHz, DMSO-d₆) δ: 9.30 (s, 1H), 7.09 (br, 1H), 7.01 (s, 1H), 4.55 (d, J=10.5 Hz, 1H), 4.01 (d, J=1.05 Hz, 1H), 3.75-3.79 (m, 1H), 2.20-2.30 (m, 1H), 1.60-2.16 (m, 8H), 1.24-1.48 (m, 2H). LC-MS: m/z 438 [M+H]⁺.

### Method J2

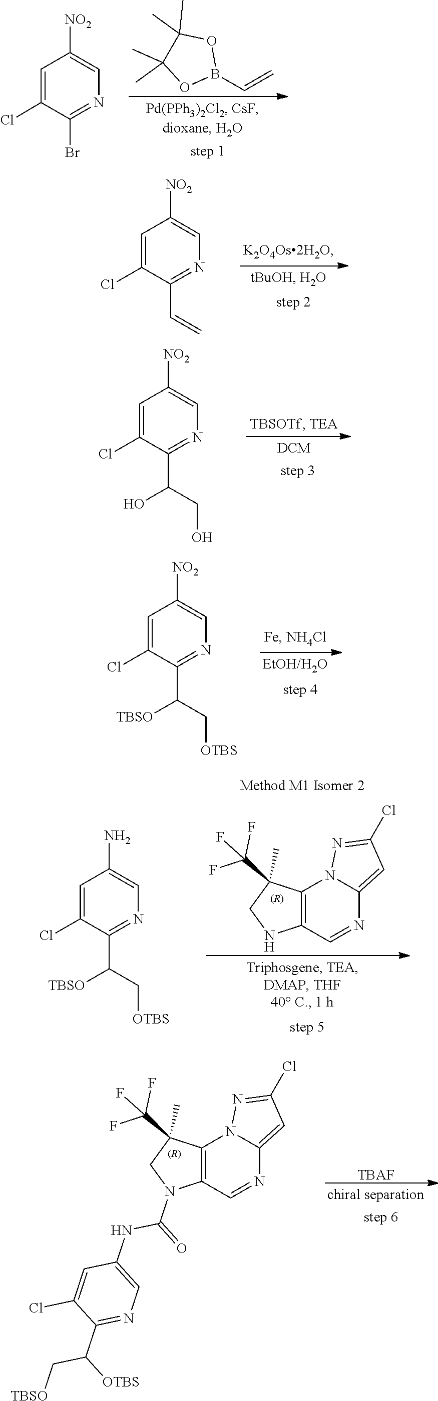

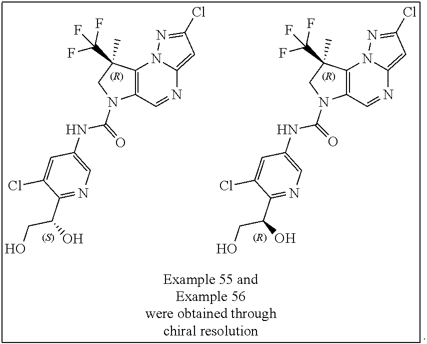

### Examples 55 and 56: Single enantiomers obtained from a mixture containing (R)-2-chloro-N-(5-chloro-6-((S)-1,2-dihydroxyethyl)pyridin-3-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(5-chloro-6-((R)-1,2-dihydroxyethyl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 3-chloro-5-nitro-2-vinylpyridine

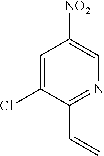

To a solution of 2-bromo-3-chloro-5-nitro-pyridine (6.0 g, 25.2 mmol) in dioxane (20 mL) and water (2 mL) were added CsF (11.5 g, 75.6 mmol), 4,4,5,5-tetramethyl-2-vinyl-1,3,2-dioxaborolane (5.8 g, 37.8 mmol) and Pd(PPh₃)₂Cl₂ (1.8 g, 2.5 mmol) under nitrogen atmosphere. The resulting mixture was stirred for 3 h at 85° C. The reaction mixture was quenched with water (200 mL). The resulting solution was extracted with ethyl acetate (3×200 mL). The combined organic layers were dried over anhydrous sodium sulfate and concentrated under vacuum. The residue was purified by column chromatography on silica gel using 50% petroleum ether and 50% ethyl acetate as eluent to afford 3-chloro-5-nitro-2-vinyl-pyridine (3.3 g, 51% yield) as a yellow oil. LC-MS: m/z 185 [M+H]⁺.

### Step 2: 1-(3-chloro-5-nitropyridin-2-yl)ethane-1,2-diol

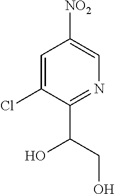 [SMILES: O=[N+]([O-])c1cnc(C(O)CO)c(Cl)c1]

To a solution of 3-chloro-5-nitro-2-vinylpyridine (3.3 g, 17.8 mmol) in t-BuOH (40 mL) and water (10 mL) were added K₂O₄Os·2H₂O (2.3 g, 6.2 mmol) and 4-methylmorpholine 4-oxide (4.2 g, 35.6 mmol). The reaction mixture was stirred at 25° C. for 2 h. The mixture was directly purified by column chromatography on silica gel using 80% petroleum ether and 20% ethyl acetate as eluent to afford 1-(3-chloro-5-nitropyridin-2-yl)ethane-1,2-diol (400 mg, 10% yield) as a yellow solid. LC-MS: m/z 219 [M+H]⁺.

### Step 3: 3-chloro-5-nitro-2-(2,2,3,3,8,8,9,9-octamethyl-4,7-dioxa-3,8-disiladecan-5-yl) pyridine

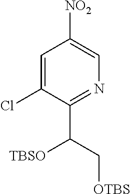 [SMILES: CC(C)(C)[Si](C)(C)OCC(O[Si](C)(C)C(C)(C)C)c1ncc([N+](=O)[O-])cc1Cl]

To a solution of 1-(3-chloro-5-nitropyridin-2-yl)ethane-1,2-diol (400 mg, 1.8 mmol) in dichloromethane (3 mL) were added TBSOTf (1.4 g, 5.4 mmol) and DIEA (820 mg, 6.3 mol) at 0° C. The reaction mixture was stirred for 3 h at 0° C. The mixture was concentrated under vacuum. The residue was diluted with water (50 mL). The resulting solution was then extracted with ethyl acetate (3×50 mL). The combined organic layers were washed with saturated aqueous NaHCO₃ (50 mL), dried over anhydrous sodium sulfate and concentrated under vacuum. The residue was purified by column chromatography on silica gel using 67% petroleum ether and 33% ethyl acetate as eluent to afford 3-chloro-5-nitro-2-(2,2,3,3,8,8,9,9-octamethyl-4,7-dioxa-3,8-disiladecan-5-yl)pyridine (200 mg, 24% yield) as a yellow solid. LC-MS: m/z 447 [M+H]⁺.

### Step 4: 5-chloro-6-(2,2,3,3,8,8,9,9-octamethyl-4,7-dioxa-3,8-disiladecan-5-yl)pyridin-3-amine

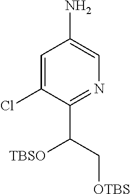 [SMILES: CC(C)(C)[Si](C)(C)OCC(O[Si](C)(C)C(C)(C)C)c1ncc(N)cc1Cl]

To a solution of 3-chloro-5-nitro-2-(2,2,3,3,8,8,9,9-octamethyl-4,7-dioxa-3,8-disiladecan-5-yl)pyridine (200 mg, 447.0 μmol) in ethanol (9 mL) and water (3 mL) were added Fe (123 mg, 2.2 mmol), NH₄Cl (95 mg, 1.7 mmol). The resulting mixture was stirred for 1 h at 80° C. The reaction mixture was quenched by the addition of water (50 mL). The resulting solution was extracted with ethyl acetate (3×50 mL). The combined organic layers were dried over anhydrous sodium sulfate and concentrated under vacuum. The residue was purified by column chromatography on silica gel using 67% petroleum ether and 33% ethyl acetate as eluent to afford 5-chloro-6-(2,2,3,3,8,8,9,9-octamethyl-4,7-dioxa-3,8-disiladecan-5-yl)pyridin-3-amine (120 mg, 64% yield) as a yellow solid. LC-MS: m/z 417 [M+H]⁺.

### Step 5: (8R)-2-chloro-N-(5-chloro-6-(2,2,3,3,8,8,9,9-octamethyl-4,7-dioxa-3,8-disiladec an-5-yl)pyridin-3-yl)-8-methyl-8-(trifluoromethyl)-7,8-dihydro-6H-pyrazolo[1,5-a]pyrrolo[2,3-e]pyrimidine-6-carboxamide

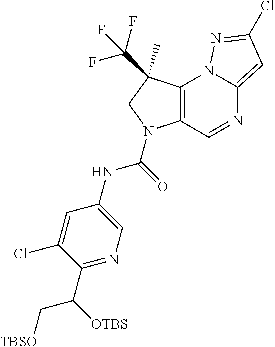 [SMILES: CC(C)(C)[Si](C)(C)OCC(O[Si](C)(C)C(C)(C)C)c1ncc(NC(=O)N2C[C@@](C)(C(F)(F)F)c3c2cnc2cc(Cl)nn32)cc1Cl]

To a mixture of 3-chloro-5-nitro-2-(2,2,3,3,8,8,9,9-octamethyl-4,7-dioxa-3,8-disiladecan-5-yl) pyridine (50 mg, 120.0 μmol) in tetrahydrofuran (3 mL) were added triphosgene (48 mg, 72.2 μmol) and TEA (41 mg, 180.1 μmol) at 25° C. The resulting mixture was stirred for 1 h at 25° C. and then filtered. The filtrate was added to a solution of Method M1 isomer 2 (33 mg, 120.0 μmol) in tetrahydrofuran (1 mL). To this solution was then added TEA (121 mg, 1.2 mmol) and N,N-Dimethylpyridin-4-amine (42 mg, 240.0 μmol). The reaction mixture was stirred for 1 h at 40° C. The residue was diluted with water (50 mL). The resulting solution was extracted with ethyl acetate (3×50 mL). The combined organic layers were washed with brine (50 mL), dried over anhydrous sodium sulfate and concentrated under vacuum. The residue was purified by column chromatography on silica gel using 20% petroleum ether and 80% ethyl acetate as eluent to afford (8R)-2-chloro-N-(5-chloro-6-(2,2,3,3,8,8,9,9-octamethyl-4,7-dioxa-3,8-disiladecan-5-yl)pyridin-3-yl)-8-methyl-8-(trifluoromethyl)-7,8-dihydro-6H-pyrazolo[1,5-a]pyrrolo[2,3-e]pyrimidine-6-carboxamide (50 mg, 53% yield) as an off-white solid. LC-MS: m/z 719 [M+H]⁺.

### Step 6: (8R)-2-chloro-N-(5-chloro-6-(1,2-dihydroxyethyl)pyridin-3-yl)-8-methyl-8-(trifluoromethyl)-7,8-dihydro-6H-pyrazolo[1,5-a]pyrrolo[2,3-e]pyrimidine-6-carboxamide

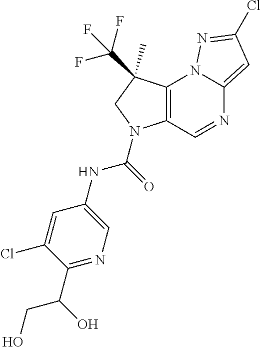 [SMILES: C[C@@]1(C(F)(F)F)CN(C(=O)Nc2cnc(C(O)CO)c(Cl)c2)c2cnc3cc(Cl)nn3c21]

To a solution of (8R)-2-chloro-N-(5-chloro-6-(2,2,3,3,8,8,9,9-octamethyl-4,7-dioxa-3,8-disiladecan-5-yl)pyridin-3-yl)-8-methyl-8-(trifluoromethyl)-7,8-dihydro-6H-pyrazolo[1,5-a]pyrrolo[2,3-e]pyrimidine-6-carboxamide (50 mg, 69.6 μmol) in tetrahydrofuran (2 mL) was added TBAF (2 mL, 2 mmol, 1 M in tetrahydrofuran) at 25° C. The resulting mixture was stirred for 4 h at 25° C. The reaction mixture was concentrated under reduced pressure. The residue was diluted with water (50 mL). The resulting solution was extracted with ethyl acetate (3×50 mL). The combined organic layers were washed with saturated aqueous NH₄Cl (3×50 mL), dried over anhydrous sodium sulfate and concentrated under vacuum. The residue was purified by Prep-TLC with ethyl acetate to afford 30 mg of crude product (90% purity). The crude product was submitted to Prep-HPLC purification and the collected fractions were lyophilized to afford (8R)-2-chloro-N-(5-chloro-6-(1,2-dihydroxyethyl)pyridin-3-yl)-8-methyl-8-(trifluoromethyl)-7,8-dihydro-6H-pyrazolo[1,5-a]pyrrolo[2,3-e]pyrimidine-6-carboxamide (20 mg, 54% yield) as a white solid. LC-MS: m/z 491 [M+H]⁺.

### Step 7: Separation of enantiomers to obtain (R)-2-chloro-N-(5-chloro-6-((S)-1,2-dihydroxyethyl)pyridin-3-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(5-chloro-6-((R)-1,2-dihydroxyethyl)pyridin-3-yl)-8-methyl-8-(trifluoromethyl)-7,8-dihydro-6H-pyrazolo[1,5-a]pyrrolo[2,3-e]pyrimidine-6-carboxamide

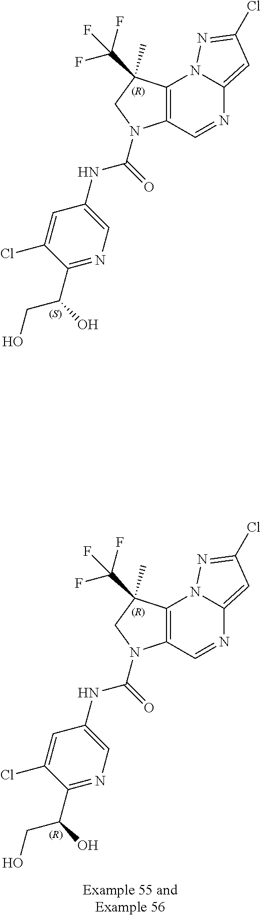

20 mg of (8R)-2-chloro-N-(5-chloro-6-(1,2-dihydroxyethyl)pyridin-3-yl)-8-methyl-8-(trifluoromethyl)-7,8-dihydro-6H-pyrazolo[1,5-a]pyrrolo[2,3-e]pyrimidine-6-carboxamide was submitted to chiral HPLC purification (Column: CHIRALPAK IF, 2×25 cm, 5 um; Mobile Phase A: MTBE(0.5% 2M NH₃-MeOH)—HPLC, Mobile Phase B:EtOH—HPLC; Flow rate: 20 mL/min; Gradient: 10 B to 10 B in 21 min; 220/254 nm; RT1: 12.1; RT2: 14.928; Injection Volume: 1.5 ml; Number Of Runs: 2). The first eluting isomer was concentrated and lyophilized to afford Example 55 as a white solid (5.0 mg, 19% yield). The second eluting isomer was concentrated and lyophilized to afford Example 56 as a white solid (4.5 mg, 18% yield). The enantiomers of Examples 55 and 56 can be prepared analogously using Method M1 isomer 1 in step 5.

Example 55: ¹H NMR (300 MHz, DMSO-d₆) δ 9.41 (s, 1H), 9.34 (s, 1H), 8.69 (d, J=2.1 Hz, 1H), 8.15 (d, J=2.1 Hz, 1H), 7.08 (s, 1H), 5.17-5.19 (m, 1H), 4.93-4.99 (m, 1H), 4.81 (d, J=11.4 Hz, 1H), 4.67-4.71 (m, 1H), 4.27 (d, J=11.4 Hz, 1H), 3.63-3.73 (m, 2H), 1.98 (s, 3H); LC-MS: m/z 491 [M+H]⁺.

Example 56: ¹H NMR (300 MHz, DMSO-d₆) δ 9.39 (s, 1H), 9.34 (s, 1H), 8.69 (d, J=2.1 Hz, 1H), 8.15 (d, J=2.1 Hz, 1H), 7.07 (s, 1H), 5.16-5.18 (m, 1H), 4.93-4.99 (m, 1H), 4.82 (d, J=11.7 Hz, 1H), 4.67-4.71 (m, 1H), 4.27 (d, J=11.7 Hz, 1H), 3.63-3.73 (m, 2H), 1.98 (s, 3H); LC-MS: m/z 491 [M+H]⁺

### Method K2

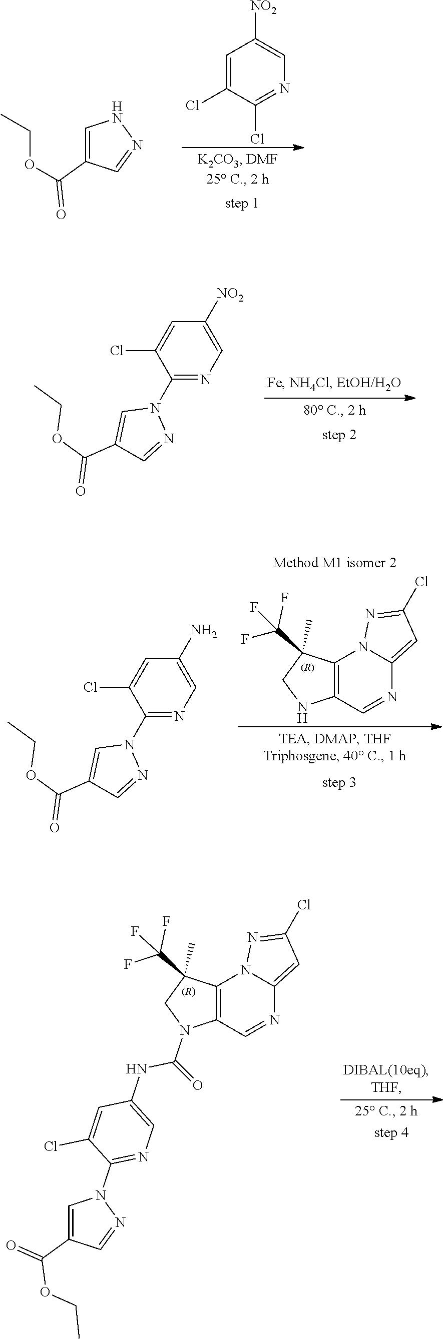

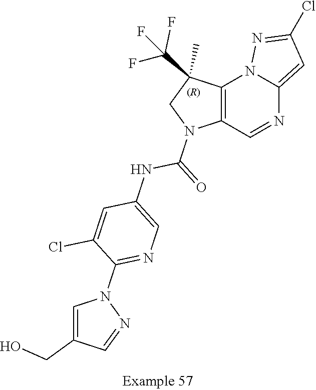

### Examples 57: (R)-2-chloro-N-(5-chloro-6-(4-(hydroxymethyl)-1H-pyrazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: ethyl 1-(3-chloro-5-nitropyridin-2-yl)-1H-pyrazole-4-carboxylate

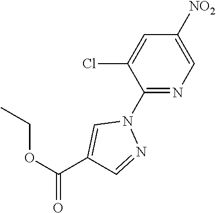 [SMILES: CCOC(=O)c1cnn(-c2ncc([N+](=O)[O-])cc2Cl)c1]

To a stirred mixture of ethyl 1H-pyrazole-4-carboxylate (1.0 g, 7.1 mmol) and 2,3-dichloro-5-nitropyridine (1.4 g, 7.1 mmol) in N,N-dimethylformamide (20 mL) was added K₂CO₃ (2.9 g, 21.4 mmol) at 25° C. The mixture was stirred for 2 h at 25° C. The reaction mixture was diluted with ethyl acetate (150 mL). The resulting solution was washed with water (2×100 mL) and brine (2×100 mL), dried over anhydrous sodium sulfate and concentrated. The residue was purified by column chromatography using 80% petroleum ether and 20% ethyl acetate as eluent to give ethyl 1-(3-chloro-5-nitropyridin-2-yl)-1H-pyrazole-4-carboxylate (1.4 g, 64% yield) as a white solid. ¹H NMR (300 MHz, Chloroform-d) δ: 9.26 (d, J=2.4 Hz, 1H), 8.86 (s, 1H), 8.76 (d, J=2.1 Hz, 1H), 8.24 (s, 1H), 4.35-4.42 (m, 2H), 1.41 (t, J=7.2 Hz, 3H). LC-MS: m/z 297 [M+H]⁺.

### Step 2: ethyl 1-(5-amino-3-chloropyridin-2-yl)-1H-pyrazole-4-carboxylate

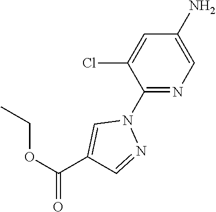 [SMILES: CCOC(=O)c1cnn(-c2ncc(N)cc2Cl)c1]

To a stirred mixture of ethyl 1-(3-chloro-5-nitropyridin-2-yl)-1H-pyrazole-4-carboxylate (500 mg, 1.7 mmol) in ethanol (15 mL) and water (5 mL) were added Fe (282 mg, 5.1 mmol) and NH₄Cl (459 mg, 8.4 mmol) at 25° C. The mixture was stirred for 1 h at 80° C. After cooled to 25° C., the reaction mixture was diluted with water (50 mL). The resulting solution was extracted with ethyl acetate (3×50 mL). The combined organic layers were dried over anhydrous sodium sulfate and concentrated under vacuum. The residue was purified by column chromatography using 50% petroleum ether and 50% ethyl acetate as eluent to afford ethyl 1-(5-amino-3-chloropyridin-2-yl)-1H-pyrazole-4-carboxylate (410 mg, 91% yield) as a white solid. ¹H NMR (400 MHz, Chloroform-d) δ: 8.35 (s, 1H), 8.11 (s, 1H), 7.86 (d, J=2.4 Hz, 1H), 7.14 (d, J=2.8 Hz, 1H), 4.29-4.35 (m, 2H), 3.86 (brs, 2H), 1.25 (t, J=6.8 Hz, 3H). LC-MS: m/z 267 [M+H]⁺.

### Step 3: ethyl (R)-1-(3-chloro-5-(2-chloro-8-methyl-8-(trifluoromethyl)-7,8-dihydro-6H-pyrazolo[1,5-a]pyrrolo[2,3-e]pyrimidine-6-carboxamido)pyridin-2-yl)-1H-pyrazole-4-carboxylate

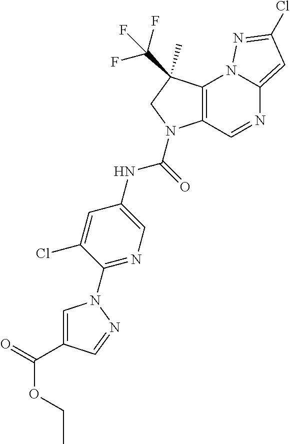 [SMILES: CCOC(=O)c1cnn(-c2ncc(NC(=O)N3C[C@@](C)(C(F)(F)F)c4c3cnc3cc(Cl)nn43)cc2Cl)c1]

To a stirred solution of Method M1 isomer 2 (50 mg, 180.7 μmol) in tetrahydrofuran (5 mL) were added triphosgene (32 mg, 108.4 μmol) and TEA (27 mg, 271.1 μmol). The reaction mixture was stirred for 30 min at 25° C. and then filtered. The filtrate was added to a solution of ethyl 1-(5-amino-3-chloropyridin-2-yl)-1H-pyrazole-4-carboxylate (96.40 mg, 361.47 μmol) in tetrahydrofuran (5 mL). To this solution were added N,N-dimethylpyridin-4-amine (44 mg, 361.5 μmol) and TEA (183 mg, 1.8 mmol). The mixture was stirred at 40° C. for 1 h. The reaction mixture was concentrated under vacuum. The residue was purified by prep-TLC using 25% petroleum ether and 75% ethyl acetate to afford ethyl (R)-1-(3-chloro-5-(2-chloro-8-methyl-8-(trifluoromethyl)-7,8-dihydro-6H-pyrazolo[1,5-a]pyrrolo[2,3-e]pyrimidine-6-carboxamido)pyridin-2-yl)-1H-pyrazole-4-carboxylate (45 mg, 43% yield) as a light-yellow solid. ¹H NMR (300 MHz, DMSO-d₆) δ: 9.63 (br, 1H), 9.34 (s, 1H), 8.77 (s, 1H), 8.69 (d, J=2.1 Hz, 1H), 8.43 (d, J=2.4 Hz, 1H), 8.14 (s, 1H), 7.06 (s, 1H), 4.83 (d, J=8.4 Hz, 1H), 4.18-4.29 (m, 3H), 1.97 (s, 3H), 1.30-1.28 (m, 3H). LC-MS: m/z 569 [M+H]⁺.

### Step 4: (R)-2-chloro-N-(5-chloro-6-(4-(hydroxymethyl)-1H-pyrazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide

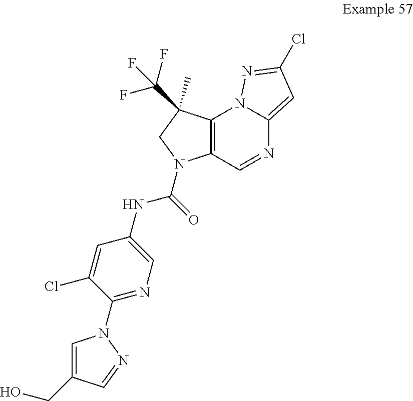 [SMILES: C[C@@]1(C(F)(F)F)CN(C(=O)Nc2cnc(-n3cc(CO)cn3)c(Cl)c2)c2cnc3cc(Cl)nn3c21]

To a stirred mixture of ethyl (R)-1-(3-chloro-5-(2-chloro-8-methyl-8-(trifluoromethyl)-7,8-dihydro-6H-pyrazolo[1,5-a]pyrrolo[2,3-e]pyrimidine-6-carboxamido)pyridin-2-yl)-1H-pyrazole-4-carboxylate (50 mg, 87.8 μmol) in tetrahydrofuran (5 mL) was added DIBAL-H (0.45 mL, 9.0 mmol, 2 M in tetrahydrofuran) dropwise at 0° C. The reaction mixture was stirred for 2 h at 25° C. The mixture was poured into ice/water (30 mL). The resulting solution was extracted with ethyl acetate (3×20 mL). The combined organic layers were dried over anhydrous sodium sulfate and concentrated under vacuum. The crude product was submitted to Prep-HPLC purification and the collected fractions were lyophilized to afford (R)-2-chloro-N-(5-chloro-6-(4-(hydroxymethyl)-1H-pyrazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide (5.3 mg, 11% yield) as a white solid. The enantiomer of Example 57 can be prepared analogously using Method M1 isomer 1.

Example 57: ¹H NMR (400 MHz, DMSO-d₆) δ: 9.37 (s, 1H), 8.60-8.62 (m, 1H), 8.38-8.43 (m, 2H), 8.06 (s, 1H), 7.71 (s, 1H), 7.07 (s, 1H), 4.96-5.02 (m, 1H), 4.79 (d, J=11.6 Hz, 1H), 4.45 (s, 2H), 4.26 (d, J=11.6 Hz, 1H), 1.97 (s, 3H). LC-MS: m/z 527 [M+H]⁺.

### Method L2

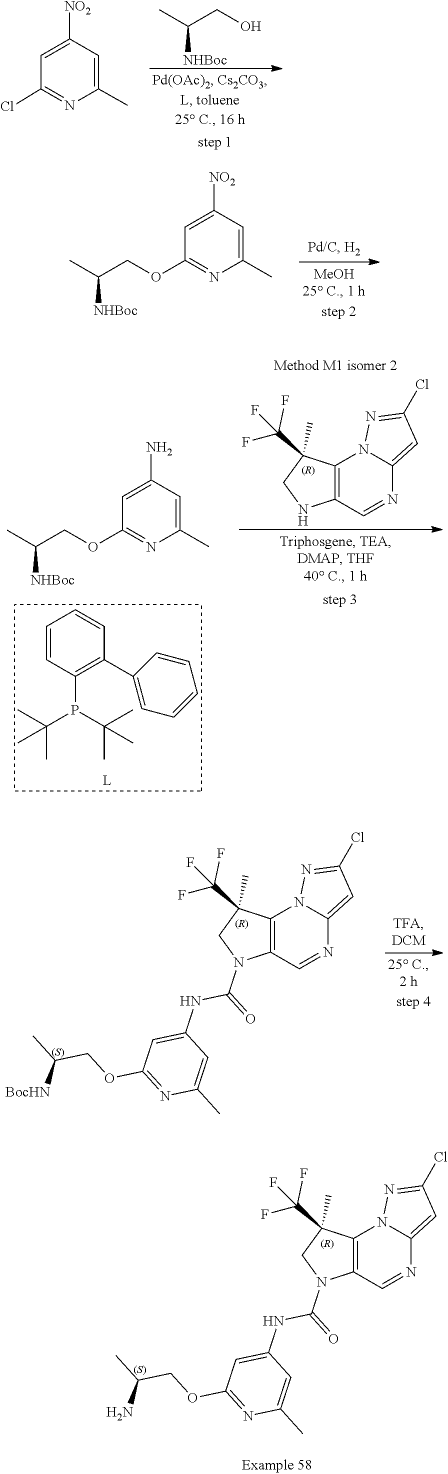

### Example 58: (R)—N-(2-((S)-2-aminopropoxy)-6-methylpyridin-4-yl)-2-chloro-8-methyl-8-(trifluoromethyl)-7,8-dihydro-6H-pyrazolo[1,5-a]pyrrolo[2,3-e]pyrimidine-6-carboxamide

### Step 1: tert-butyl (S)-(1-((6-methyl-4-nitropyridin-2-yl)oxy)propan-2-yl)carbamate

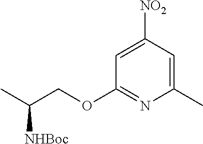 [SMILES: Cc1cc([N+](=O)[O-])cc(OC[C@H](C)NC(=O)OC(C)(C)C)n1]

To a stirred mixture of 2-chloro-6-methyl-4-nitropyridine (5 g, 29.0 mmol) in dioxane (50 mL) were added tert-butyl (S)-(1-hydroxypropan-2-yl)carbamate (10.2 g, 58.0 mmol), Pd(OAc)₂ (1.3 g, 5.8 mmol), [1,1′-biphenyl]-2-yldi-tert-butylphosphane (2.2 g, 7.2 mmol) and Cs₂CO₃ (18.9 g, 57.8 mmol). The reaction mixture was stirred at 25° C. for 16 h under nitrogen. The resulting mixture was concentrated under vacuum. The residue was purified by column chromatography on silica gel using 85% petroleum ether and 15% ethyl acetate as eluent to afford tert-butyl (S)-(1-((6-methyl-4-nitropyridin-2-yl)oxy)propan-2-yl)carbamate (4.6 g, 50% yield) as a yellow solid. ¹H NMR (300 MHz, DMSO-d₆) δ: 7.58 (d, J=1.8 Hz, 1H), 7.25 (d, J=1.8 Hz, 1H), 6.86 (d, J=8.3 Hz, 1H), 4.10-4.23 (m, 2H), 3.81-3.91 (m, 1H), 2.52 (d, J=0.6 Hz, 3H), 1.36 (s, 9H), 1.11 (d, J=6.8 Hz, 3H). LC-MS: m/z 312 [M+H]⁺

### Step 2: tert-butyl (S)-(1-((4-amino-6-methylpyridin-2-yl)oxy)propan-2-yl)carbamate

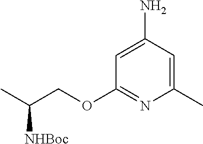 [SMILES: Cc1cc(N)cc(OC[C@H](C)NC(=O)OC(C)(C)C)n1]

To a stirred solution of tert-butyl (S)-(1-((6-methyl-4-nitropyridin-2-yl)oxy)propan-2-yl)carbamate (1 g, 3.2 mmol) in methanol (200 mL) was added Pd/C (171 mg, 10%). The reaction mixture was stirred at 25° C. under one atmosphere of hydrogen for 1 h. The reaction mixture was filtered, and the filtrate was concentrated under vacuum. The residue was purified by column chromatography on silica gel using 50% petroleum ether and 50% ethyl acetate as eluent to afford tert-butyl (S)-(1-((4-amino-6-methylpyridin-2-yl)oxy)propan-2-yl)carbamate (700 mg, 75% yield) as a yellow oil. ¹H NMR (300 MHz, DMSO-d) 6:6.88 (d, J=9 Hz, 1H), 6.01 (d, J=3 Hz, 1H), 5.81 (s, 2H), 5.62 (d, J=3 Hz, 1H), 3.91-3.95 (m, 2H), 3.67-3.82 (m, 1H), 2.14 (s, 3H), 1.38 (s, 9H), 1.06 (d, J=6 Hz, 3H). LC-MS: m/z 282 [M+H]⁺.

### Step 3: tert-butyl ((S)-1-((4-((R)-2-chloro-8-methyl-8-(trifluoromethyl)-7,8-dihydro-6H-pyrazolo[1,5-a]pyrrolo[2,3-e]pyrimidine-6-carboxamido)-6-methylpyridin-2-yl)oxy)propan-2-yl)carbamate

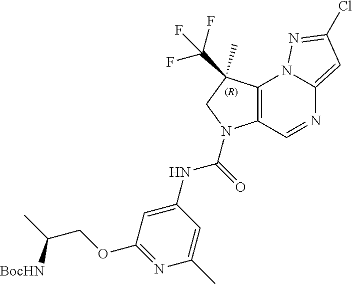

To a stirred solution of tert-butyl (S)-(1-((4-amino-6-methylpyridin-2-yl)oxy)propan-2-yl)carbamate (62 mg, 216.9 μmol) in tetrahydrofuran (8 mL) were added triphosgene (26 mg, 86.8 μmol) and TEA (22 mg, 21.8 μmol). The resulting mixture was stirred for 0.5 h at 28° C. and then filtered. The filtrate was added to a solution of Method M1 isomer 2 (40 mg, 145.4 μmol) in tetrahydrofuran (1 mL). To this solution were added N,N-dimethylpyridin-4-amine (36 mg, 290.9 μmol) and TEA (147 mg, 1.5 mmol). The mixture was stirred at 40° C. for 1 h. The mixture was quenched with water (20 mL). The resulting solution was extracted with ethyl acetate (3×40 mL). The combined organic layers were washed with brine (50 mL) and dried over anhydrous sodium sulfate and concentrated under vacuum. The residue was purified by column chromatography using 50% petroleum ether and 50% ethyl acetate as eluent to afford tert-butyl ((S)-1-((4-((R)-2-chloro-8-methyl-8-(trifluoromethyl)-7,8-dihydro-6H-pyrazolo[1,5-a]pyrrolo[2,3-e]pyrimidine-6-carboxamido)-6-methylpyridin-2-yl)oxy)propan-2-yl)carbamate (50 mg, 47% yield) as a white solid. LC-MS: m/z 584 [M+H]⁺

### Step 4: (R)—N-(2-((S)-2-aminopropoxy)-6-methylpyridin-4-yl)-2-chloro-8-methyl-8-(trifluoromethyl)-7,8-dihydro-6H-pyrazolo[1,5-a]pyrrolo[2,3-e]pyrimidine-6-carboxamide

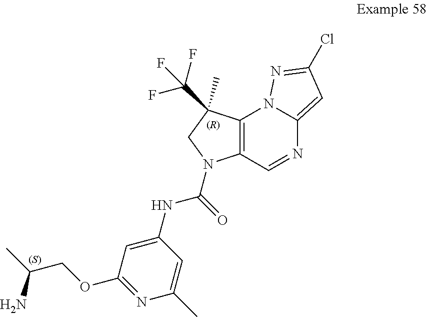

To a stirred solution of tert-butyl ((S)-1-((4-((R)-2-chloro-8-methyl-8-(trifluoromethyl)-7,8-dihydro-6H-pyrazolo[1,5-a]pyrrolo[2,3-e]pyrimidine-6-carboxamido)-6-methylpyridin-2-yl)oxy)propan-2-yl)carbamate (40 mg, 68.6 μmol) in dichloromethane (4.8 mL) was added TFA (1.2 mL). The mixture was stirred at 25° C. for 2 h. The resulting mixture was concentrated under vacuum. The residue was added saturated aqueous NaHCO₃ (40 mL). The resulting solution was extracted with ethyl acetate (3×40 mL). The combined organic layers were washed with brine (50 mL) and dried over anhydrous sodium sulfate and concentrated under vacuum. The residue was purified by Prep-HPLC and the collected fractions were lyophilized to give (R)—N-(2-((S)-2-aminopropoxy)-6-methylpyridin-4-yl)-2-chloro-8-methyl-8-(trifluoromethyl)-7,8-dihydro-6H-pyrazolo[1,5-a]pyrrolo[2,3-e]pyrimidine-6-carboxamide (12.5 mg, 37% yield) as a white solid. The epimer of Example 58 ((S)—N-(2-((S)-2-aminopropoxy)-6-methylpyridin-4-yl)-2-chloro-8-methyl-8-(trifluoromethyl)-7,8-dihydro-6H-pyrazolo[1,5-a]pyrrolo[2,3-e]pyrimidine-6-carboxamide) can be prepared analogously using Method M1 isomer 1.

Example 58: ¹H NMR (400 MHz, DMSO-d₆) δ: 9.32 (s, 1H), 7.06 (s, 1H), 7.01 (d, J=0.8 Hz, 1H), 6.94 (d, J=1.2 Hz, 1H), 4.82-4.85 (m, 1H), 4.21-4.24 (m, 1H), 3.94-3.96 (m, 2H), 3.10-3.12 (m, 1H), 2.33 (s, 3H), 1.96 (s, 3H), 1.04 (d, J=6.4 Hz, 3H). LC-MS: m/z 484 [M+H]⁺.

### Method M2

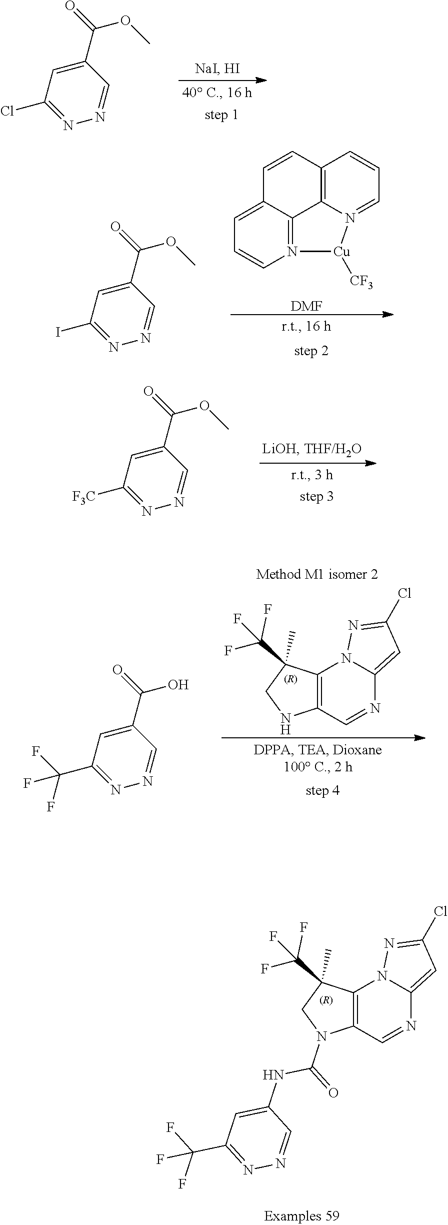

### Examples 59: (R)-11-chloro-3-methyl-3-(trifluoromethyl)-N-[6-(trifluoromethyl) pyridazin-4-yl]-1,5,8,12-tetrazatricyclo[7.3.0.02,6]dodeca-2(6),7,9,11-tetraene-5-carboxamide

### Step 1: methyl 6-iodopyridazine-4-carboxylate

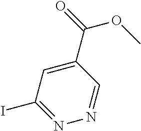

To a stirred solution of methyl 6-chloropyridazine-4-carboxylate (3.0 g, 17.4 mmol) in hydriodic acid (30 mL, 57%) was added NaI (3.4 g, 23.0 mmol). The reaction was stirred at 40° C. for 16 h. The mixture was cooled to room temperature. The pH was adjusted to 7 with saturated aqueous NaHCO₃ solution. The resulting solution was extracted with ethyl acetate (3×150 mL). The combined organic layers were washed with saturated aqueous NH₄Cl solution (150 mL), brine (150 mL), dried over anhydrous sodium sulfate and concentrated under vacuum to afford methyl 6-iodopyridazine-4-carboxylate (2.5 g, 54% yield) as a yellow solid. ¹H NMR (300 MHz, Chloroform-d) δ: 9.61 (d, J=1.8 Hz, 1H), 8.39 (d, J=1.8 Hz, 1H), 4.04 (s, 3H). LC-MS: m/z 265 [M+H]⁺.

### Step 2: methyl 6-(trifluoromethyl)pyridazine-4-carboxylate

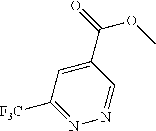

To a stirred solution of methyl 6-iodopyridazine-4-carboxylate (200 mg, 758 μmol) in N,N-dimethylformamide (4 mL) was added (1,10-Phenanthroline)(trifluoromethyl)copper (308 mg, 985 μmol) in N,N-dimethylformamide (5 mL) under nitrogen. The resulting solution was stirred for 16 h at 25° C. (exclusion of light). The reaction mixture was diluted with ethyl acetate (50 mL) and filtered through the celite. The organic layer was washed with water (20 mL), brine (20 mL), dried over anhydrous sodium sulfate and concentrated under vacuum. The residue was purified by column chromatography using 70% petroleum ether and 30% ethyl acetate to afford methyl 6-(trifluoromethyl) pyridazine-4-carboxylate (60 mg, 38% yield) as a white solid. ¹H NMR (300 MHz, Chloroform-d) δ: 9.87 (d, J=1.8 Hz, 1H), 8.35 (d, J=1.8 Hz, 1H), 4.09 (s, 3H). LC-MS: m/z 207 [M+H]⁺.

### Step 3: 6-(trifluoromethyl)pyridazine-4-carboxylic acid

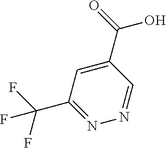 [SMILES: O=C(O)c1cnnc(C(F)(F)F)c1]

To a stirred solution of methyl 6-(trifluoromethyl)pyridazine-4-carboxylate (70 mg, 340 μmol) in tetrahydrofuran (1 mL) and water (1 mL) was added LiOH (20 mg, 849 μmol). The reaction mixture was stirred at 25° C. for 3 h. The mixture was concentrated. The residue was diluted with water (20 mL). The pH was adjusted to 2 with HCl (6 N). The mixture was extracted with ethyl acetate (3×20 mL). The combined organic layers were dried over anhydrous sodium sulfate and concentrated under vacuum to afford 6-(trifluoromethyl)pyridazine-4-carboxylic acid (35 mg, 53% yield) as a yellow solid. ¹H NMR (300 MHz, Chloroform-d) δ: 9.95 (d, J=1.8 Hz, 1H), 8.42 (d, J=1.8 Hz, 1H). LC-MS: m/z 193 [M+H]⁺.

### Step 4: (R)-2-chloro-8-methyl-8-(trifluoromethyl)-N-(6-(trifluoromethyl)pyridazin-4-yl)-7,8-dihydro-6H-pyrazolo[1,5-a]pyrrolo[2,3-e]pyrimidine-6-carboxamide

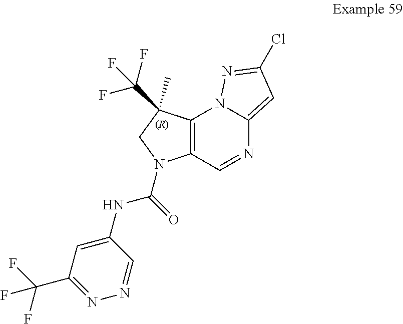

To a stirred solution of 6-(trifluoromethyl)pyridazine-4-carboxylic acid (12 mg, 62 μmol) in dioxane (1 mL) was added DPPA (21 mg, 75 μmol) and TEA (32 mg, 312 μmol). The solution was stirred at 25° C. for 30 min. Method M1 isomer 2 (10 mg, 37 μmol) was added and the mixture was stirred for 2 h at 100° C. The reaction mixture was cooled to 25° C. The reaction mixture was quenched with water (10 mL). The resulting solution was extracted with ethyl acetate (3×10 mL). The combined organic layers were dried over anhydrous sodium sulfate and concentrated under vacuum. The afforded crude was purified by Prep-HPLC purification and the collected fractions were lyophilized to give (R)-2-chloro-8-methyl-8-(trifluoromethyl)-N-(6-(trifluoromethyl)pyridazin-4-yl)-7,8-dihydro-6H-pyrazolo[1,5-a]pyrrolo[2,3-e]pyrimidine-6-carboxamide (7.1 mg, 24% yield) as a white solid. The enantiomer of Example 59 can be prepared analogously using Method M1 isomer 1.

Example 59: ¹H NMR (300 MHz, Methanol-d₄) δ: 9.58 (d, J=2.4 Hz, 1H), 9.43 (s, 1H), 8.48 (d, J=2.4 Hz, 1H), 6.83 (s, 1H), 4.86-4.92 (m, 1H), 4.25-4.29 (m, 1H), 2.07 (s, 3H). LC-MS: m/z 466 [M+H]⁺.

### Example 60: (R)-2-chloro-N-(2-(difluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

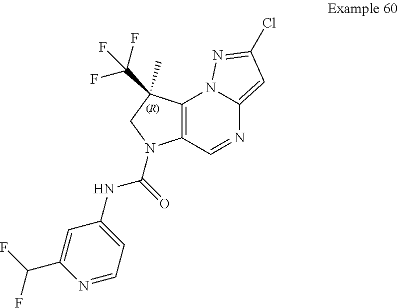

The title compound was prepared according to Method M1 Step 2 by using 2-(difluoromethyl)pyridin-4-amine and Method M1 Isomer 2. The enantiomer of Example 60 can be prepared analogously using Method M1 isomer 1.

Example 60: ¹H NMR (400 MHz, Chloroform-d) δ: 9.41 (s, 1H), 8.59 (d, J=5.6 Hz, 1H), 7.69-7.72 (m, 2H), 6.80-6.49 (m, 3H), 4.57 (d, J=10.4 Hz, 1H), 4.06 (d, J=10.4 Hz, 1H), 2.08 (s, 3H). LC-MS: m/z 447 [M+H]⁺.

### Method N2

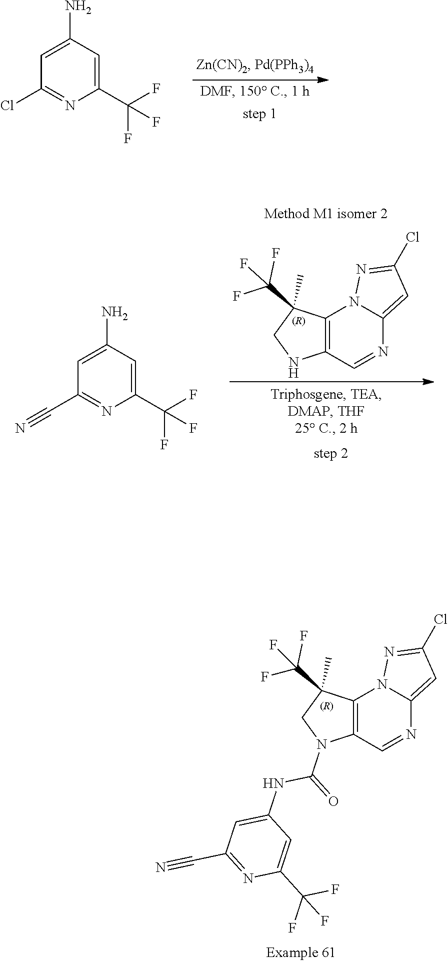

### Example 61: (R)-2-chloro-N-(2-cyano-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

### Step 1: 4-amino-6-(trifluoromethyl)picolinonitrile

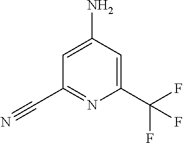 [SMILES: N#Cc1cc(N)cc(C(F)(F)F)n1]

To a stirred solution of 2-chloro-6-(trifluoromethyl)pyridin-4-amine (250 mg, 1.3 mmol) in N,N-dimethylformamide (2 mL) were added Zn(CN)₂ (149 mg, 1.3 mmol) and Pd(PPh₃)₄ (73 mg, 63.6 μmol). The reaction mixture was stirred at 150° C. for 1 h under nitrogen. The reaction mixture was quenched with water (10 mL). The resulting solution was extracted with ethyl acetate (3×10 mL). The combined organic layers were dried over anhydrous sodium sulfate and concentrated under vacuum. The residue was purified by column chromatography on silica gel using 20% petroleum ether and 80% ethyl acetate as eluent to afford 4-amino-6-(trifluoromethyl)picolinonitrile (170 mg, 71% yield) as a white solid. ¹H NMR (400 MHz, DMSO-d) δ: 7.21 (br, 2H), 7.14 (d, J=2.0 Hz, 1H), 7.09 (d, J=2.0 Hz, 1H). LC-MS: m/z 188 [M+H]⁺.

### Step 2: (R)-2-chloro-N-(2-cyano-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

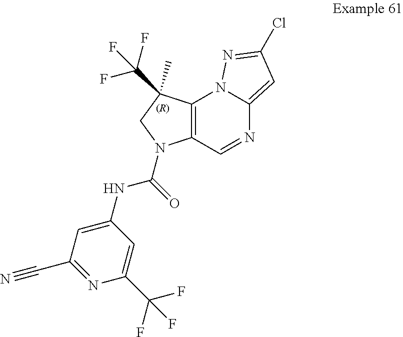

To a stirred solution of Method M1 isomer 2 (20 mg, 72.3 μmol) in tetrahydrofuran (2 mL) were added triphosgene (13 mg, 43.4 μmol) and TEA (11 mg, 108.4 μmol). The resulting mixture was stirred for 0.5 h at 25° C. and then filtered. The filtrate was added to a solution of 4-amino-6-(trifluoromethyl)picolinonitrile (27 mg, 144.6 μmol) in tetrahydrofuran (1 mL). To this solution were added N,N-dimethylpyridin-4-amine (18 mg, 144.6 μmol) and TEA (73 mg, 723.0 μmol). The mixture was stirred at 25° C. for 2 h. The mixture was concentrated under vacuum. The residue was purified by Prep-HPLC and the collected fractions were lyophilized to afford (R)-2-chloro-N-(2-cyano-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide (5.2 mg, 14% yield) as a white solid. The enantiomer of Example 61 can be prepared analogously using Method M1 isomer 1.

Example 61: ¹H NMR (300 MHz, DMSO-d₆) δ: 10.12 (s, 1H), 9.34 (s, 1H), 8.39 (d, J=1.8 Hz, 1H), 8.31 (d, J=1.8 Hz, 1H), 7.11 (s, 1H), 4.86 (d, J=11.7 Hz, 1H), 4.30 (d, J=11.7 Hz, 1H), 1.98 (s, 3H). LC-MS: m/z 490 [M+H]⁺.

### Method O2

### Method P2

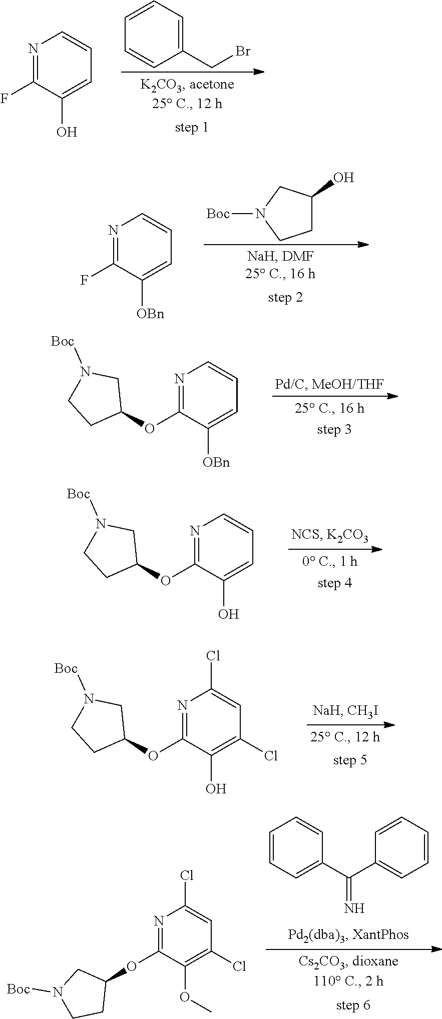

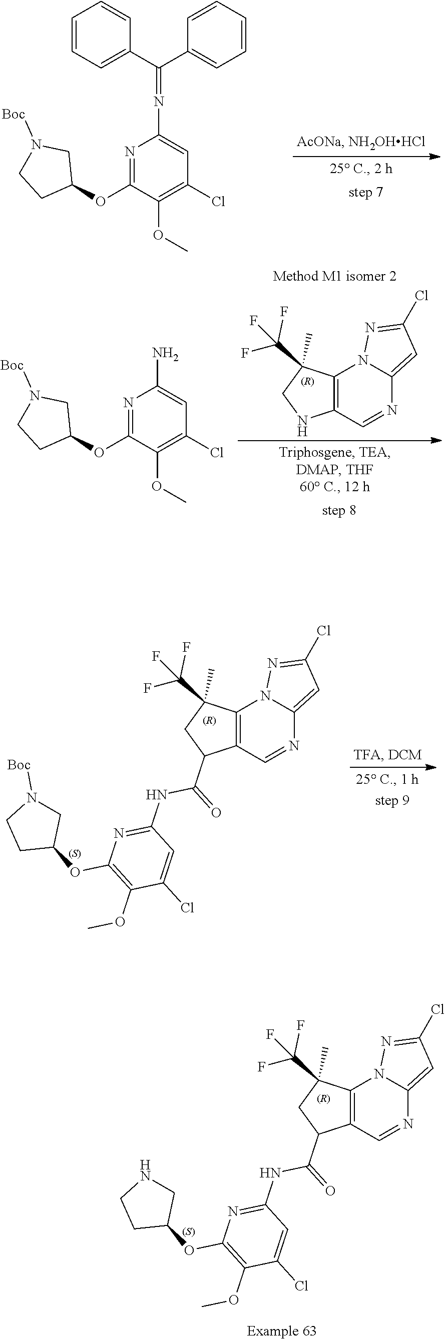

### Examples 63: (R)-2-chloro-N-(4-chloro-5-methoxy-6-(((S)-pyrrolidin-3-yl)oxy)pyridin-2-yl)-8-methyl-8-(trifluoromethyl)-7,8-dihydro-6H-pyrazolo[1,5-a]pyrrolo[2,3-e]pyrimidine-6-carboxamide

### Step 1: 3-(benzyloxy)-2-fluoropyridine

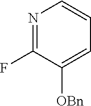 [SMILES: Fc1ncccc1OCc1ccccc1]

Into a 250 mL round bottom flask was placed acetone (100 mL) and 2-fluoropyridin-3-ol (10.0 g, 88.4 mmol). (bromomethyl)benzene (22.7 g, 132.6 mmol) and K₂CO₃ (24.4 g, 176.9 mmol) were added into the solution at 25° C. The reaction mixture was stirred at 25° C. for 12 h. The reaction mixture was concentrated under vacuum. The residue was diluted with water (300 mL). The resulting solution was extracted with ethyl acetate (3×200 mL). The combined organic layers were concentrated under vacuum. The residue was purified by column chromatography using 70% petroleum ether and 30% ethyl acetate to afford 3-(benzyloxy)-2-fluoropyridine (15.0 g, 81% yield) as a white solid. ¹H NMR (300 MHz, Chloroform-d) δ: 7.77-7.79 (m, 1H), 7.35-7.48 (m, 5H), 7.29-7.33 (m, 1H), 7.07-7.12 (m, 1H), 5.18 (s, 2H). LC-MS: m/z 204 [M+H]⁺.

### Step 2: tert-butyl (S)-3-((3-(benzyloxy)pyridin-2-yl)oxy)pyrrolidine-1-carboxylate

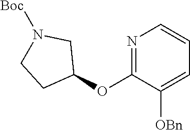 [SMILES: CC(C)(C)OC(=O)N1CC[C@H](Oc2ncccc2OCc2ccccc2)C1]

To a stirred solution of tert-butyl (S)-3-hydroxypyrrolidine-1-carboxylate (4.1 g, 21.7 mmol) in N,N-dimethylformamide (10 mL) was added NaH (1.1 g, 29.5 mmol, 60% in mineral oil) in batches at 0° C. The reaction was stirred for 0.5 h and 3-(benzyloxy)-2-fluoropyridine (4.0 g, 19.7 mmol) was added at 0° C. The reaction mixture was stirred at 25° C. for 16 h. The mixture was diluted with water (200 mL). The resulting solution was extracted with ethyl acetate (3×150 mL). The combined organic layers were dried over anhydrous sodium sulfate and concentrated under vacuum. The residue was purified by column chromatography using 50% petroleum ether and 50% ethyl acetate as eluent to afford tert-butyl (S)-3-((3-(benzyloxy)pyridin-2-yl)oxy)pyrrolidine-1-carboxylate (7.0 g, 91% yield) as a white solid. ¹H NMR (400 MHz, DMSO-d₆) δ 7.70 (dd, J=5.0, 1.5 Hz, 1H), 7.43-7.25 (m, 6H), 6.89 (dd, J=7.8, 5.0 Hz, 1H), 5.44-5.53 (m, 1H), 5.12 (s, 2H), 3.58-3.62 (m, 1H), 3.33-3.47 (m, 3H), 2.00-2.22 ((m, 2H), 1.37 (d, J=5.3 Hz, 9H). LC-MS: m/z 371 [M+H]⁺

### Step 3: tert-butyl (S)-3-((3-hydroxypyridin-2-yl)oxy)pyrrolidine-1-carboxylate

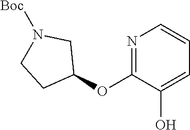 [SMILES: CC(C)(C)OC(=O)N1CC[C@H](Oc2ncccc2O)C1]

Into a 500 mL round bottom flask were placed tert-butyl (S)-3-((3-(benzyloxy)pyridin-2-yl)oxy)pyrrolidine-1-carboxylate (18.0 g, 48.6 mmol), tetrahydrofuran (140 mL), methanol (140 mL) and Pd/C (1.8 g, 10%). The flask was evacuated and flushed with nitrogen, followed by flushing with hydrogen. The mixture was stirred for 16 h at 25° C. under an atmosphere of hydrogen. The solid was filtered out. The filtrate was concentrated under vacuum. This resulted in crude tert-butyl (S)-3-((3-hydroxypyridin-2-yl)oxy)pyrrolidine-1-carboxylate (10.0 g, 72% yield) as an off-white solid. ¹H NMR (400 MHz, DMSO-d₄) S 9.42 (s, 1H), 7.56 (dd, J=4.9, 1.6 Hz, 1H), 7.08 (dd, J=7.7, 1.6 Hz, 1H), 6.79 (dd, J=7.6, 4.9 Hz, 1H), 5.40-5.45 (m, 1H), 3.57-3.60 (m, 1H), 3.30-3.46 (m, 3H), 2.01-2.15 (m, 2H), 1.37 (d, J=8.2 Hz, 9H). LC-MS: m/z 281 [M+H]⁺

### Step 4: tert-butyl (S)-3-((4,6-dichloro-3-hydroxypyridin-2-yl)oxy)pyrrolidine-1-carboxylate

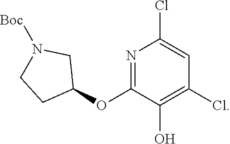 [SMILES: CC(C)(C)OC(=O)N1CC[C@H](Oc2nc(Cl)cc(Cl)c2O)C1]

To a stirred mixture of tert-butyl (S)-3-((3-hydroxypyridin-2-yl)oxy)pyrrolidine-1-carboxylate (6.0 g, 19.1 mmol) in water (10 mL) and tetrahydrofuran (18 mL) were added K₂CO₃ (13.2 g, 95.3 mmol) and 1-chloropyrrolidine-2,5-dione (12.7 g, 95.3 mmol) at 0° C. The reaction mixture was stirred at 0° C. for 1 h. The reaction mixture was diluted with water (50 mL) The pH was adjusted to 6-7 with HCl (2 N). The resulting mixture was extracted with ethyl acetate (3×100 mL). The combined organic layers were dried over anhydrous sodium sulfate and concentrated under vacuum to afford tert-butyl (S)-3-((4,6-dichloro-3-hydroxypyridin-2-yl)oxy)pyrrolidine-1-carboxylate (3.0 g, crude) as a white solid. LC-MS: m/z 349 [M+H]⁺

### Step 5: tert-butyl (S)-3-((4,6-dichloro-3-methoxypyridin-2-yl)oxy)pyrrolidine-1-carboxylate

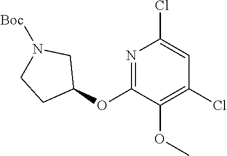 [SMILES: COc1c(Cl)cc(Cl)nc1O[C@H]1CCN(C(=O)OC(C)(C)C)C1]

To a stirred solution of tert-butyl (S)-3-((4,6-dichloro-3-hydroxypyridin-2-yl)oxy)pyrrolidine-1-carboxylate (2.0 g, 5.7 mmol) in N,N-dimethylformamide (40 mL) was added NaH (0.4 g, 11.5 mmol, 60% in mineral oil) in portions at 0° C. MeI (1.6 g, 11.5 mmol) was added into the mixture. The reaction mixture was warmed up to 25° C. and stirred for 12 h. The mixture was poured into ice/water (80 mL). The resulting solution was extracted with ethyl acetate (3×100 mL). The combined organic layers were dried over anhydrous sodium sulfate and concentrated under vacuum. The residue was purified by column chromatography using 80% petroleum ether and 20% ethyl acetate as eluent to afford tert-butyl (S)-3-((4,6-dichloro-3-methoxypyridin-2-yl)oxy)pyrrolidine-1-carboxylate (1.0 g, 38% yield) as a white solid. LC-MS: m/z 363 [M+H]⁺.

### Step 6: tert-butyl (S)-3-((4-chloro-6-((diphenylmethylene)amino)-3-methoxypyridin-2-yl)oxy)pyrrolidine-1-carboxylate

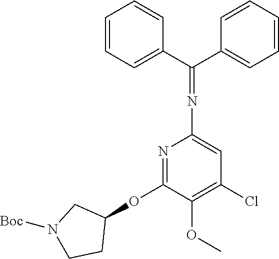 [SMILES: COc1c(Cl)cc(N=C(c2ccccc2)c2ccccc2)nc1O[C@H]1CCN(C(=O)OC(C)(C)C)C1]

Into a 40 mL vial were placed tert-butyl (S)-3-((4,6-dichloro-3-methoxypyridin-2-yl)oxy)pyrrolidine-1-carboxylate (0.4 g, 1.1 mmol), diphenylmethanimine (0.2 g, 1.1 mmol) and dioxane (20 mL) were added Xantphos (0.2 g, 330.4 μmol), Pd₂(dba)₃ (0.2 g, 220.2 μmol) and Cs₂CO₃ (1.1 g, 3.3 mmol) under nitrogen atmosphere. The resulting mixture was stirred at 110° C. for 2 h. The solid was filtered out. The filtrate was concentrated under vacuum. The residue was purified by column chromatography using 60% petroleum ether and 40% ethyl acetate as eluent to afford tert-butyl (S)-3-((4-chloro-6-((diphenylmethylene)amino)-3-methoxypyridin-2-yl)oxy)pyrrolidine-1-carboxylate (0.4 g, 78.8% yield) as a yellow solid. LC-MS: m/z 508 [M+H]⁺

### Step 7: tert-butyl (S)-3-((6-amino-4-chloro-3-methoxypyridin-2-yl)oxy)pyrrolidine-1-carboxylate

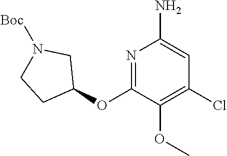 [SMILES: COc1c(Cl)cc(N)nc1O[C@H]1CCN(C(=O)OC(C)(C)C)C1]

Into a 100 mL round bottom flask were placed tert-butyl (S)-3-((4-chloro-6-((diphenylmethylene)amino)-3-methoxypyridin-2-yl)oxy)pyrrolidine-1-carboxylate (0.4 g, 826.8 μmol), hydroxylamine hydrochloride (0.1 g, 1.7 mmol), sodium acetate (0.3 g, 2.1 mmol) and methanol (20 mL). The mixture was stirred for 2 h at 25° C. The mixture was concentrated under vacuum. The residue was purified by column chromatography using 60% petroleum ether and 40% ethyl acetate as eluent to afford tert-butyl (S)-3-((6-amino-4-chloro-3-methoxypyridin-2-yl)oxy)pyrrolidine-1-carboxylate (0.3 g, 88% yield) as a yellow solid. LC-MS: m/z 344 [M+H]⁺.

### Step 8: tert-butyl (S)-3-((4-chloro-6-((R)-2-chloro-8-methyl-8-(trifluoromethyl)-7,8-dihydro-6H-pyrazolo[1,5-a]pyrrolo[2,3-e]pyrimidine-6-carboxamido)-3-methoxypyridin-2-yl)oxy)pyrrolidine-1-carboxylate

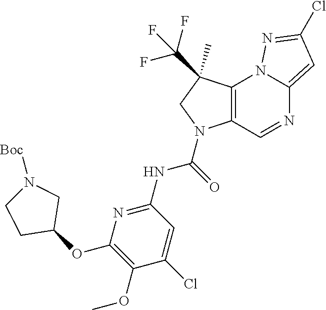 [SMILES: COc1c(Cl)cc(NC(=O)N2C[C@@](C)(C(F)(F)F)c3c2cnc2cc(Cl)nn32)nc1O[C@H]1CCN(C(=O)OC(C)(C)C)C1]

To a stirred solution of Method M1 isomer 2 (10 mg, 36.2 μmol) in tetrahydrofuran (4 mL) were added triphosgene (6 mg, 21.7 μmol) and TEA (6 mg, 54.2 μmol) at 0° C. The resulting mixture was stirred for 0.5 h at 25° C. and then filtered. The filtrate was added to a solution of tert-butyl (S)-3-((6-amino-4-chloro-3-methoxypyridin-2-yl)oxy)pyrrolidine-1-carboxylate (15 mg, 43.4 μmol) in tetrahydrofuran (2 mL). To this solution was then added N,N-dimethylpyridin-4-amine (9 mg, 72.3 μmol) and TEA (37 mg, 361.5 μmol). The mixture was stirred at 60° C. for 12 h. The mixture was poured into water (10 mL) and extracted with ethyl acetate (3×10 mL). The combined organic layers were washed with brine (20 mL), dried over anhydrous sodium sulfate and concentrated under vacuum. The residue was purified by Prep-TLC using 60% dichloromethane and 40% methanol as eluent to afford tert-butyl (S)-3-((4-chloro-6-((R)-2-chloro-8-methyl-8-(trifluoromethyl)-7,8-dihydro-6H-pyrazolo[1,5-a]pyrrolo[2,3-e]pyrimidine-6-carboxamido)-3-methoxypyridin-2-yl)oxy)pyrrolidine-1-carboxylate (20 mg, 42% yield) as a yellow oil. LC-MS: m/z 646 [M+H]⁺.

### Step 9: (R)-2-chloro-N-(4-chloro-5-methoxy-6-(((S)-pyrrolidin-3-yl)oxy)pyridin-2-yl)-8-methyl-8-(trifluoromethyl)-7,8-dihydro-6H-pyrazolo[1,5-a]pyrrolo[2,3-e]pyrimidine-6-carboxamide

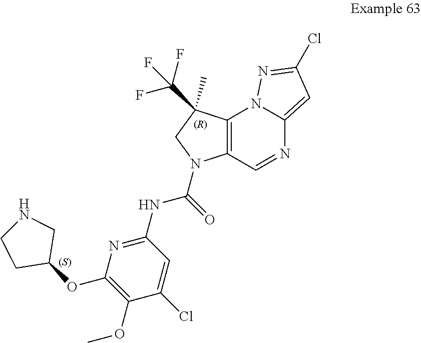

To a stirred solution of tert-butyl (S)-3-((4-chloro-6-((R)-2-chloro-8-methyl-8-(trifluoromethyl)-7,8-dihydro-6H-pyrazolo[1,5-a]pyrrolo[2,3-e]pyrimidine-6-carboxamido)-3-methoxypyridin-2-yl)oxy)pyrrolidine-1-carboxylate (20 mg, 30.9 μmol) in dichloromethane (9 mL) was added TFA (1.9 g, 1.3 mL). The mixture was stirred at 25° C. for 1 h. The resulting mixture was concentrated under reduced pressure. The residue was purified by Prep-HPLC and the collected fractions were lyophilized to give (R)-2-chloro-N-(4-chloro-5-methoxy-6-(((S)-pyrrolidin-3-yl)oxy)pyridin-2-yl)-8-methyl-8-(trifluoromethyl)-7,8-dihydro-6H-pyrazolo[1,5-a]pyrrolo[2,3-e]pyrimidine-6-carboxamide (6 mg, 14.15% yield) as a white solid. The epimer of Example 63 ((S)-2-chloro-N-(4-chloro-5-methoxy-6-(((S)-pyrrolidin-3-yl)oxy)pyridin-2-yl)-8-methyl-8-(trifluoromethyl)-7,8-dihydro-6H-pyrazolo[1,5-a]pyrrolo[2,3-e]pyrimidine-6-carboxamide) can be prepared analogously using Method M1 isomer 1.

Example 63: ¹H NMR (400 MHz, DMSO-d₆) δ: 9.29 (s, 1H), 8.27 (s, 1H), 7.52 (s, 1H), 7.04 (s, 1H), 5.51 (s, 1H), 4.93 (d, J=11.6 Hz, 1H), 4.22 (d, J=11.6 Hz, 1H), 3.75 (s, 3H), 2.92-3.07 (m, 4H), 2.06-2.17 (m, 2H), 1.93 (s, 3H). LC-MS: m/z 546 [M+H]⁺

### Example 64: (R)-2-chloro-N-(1-(difluoromethyl)-1H-pyrazol-4-yl)-8-methyl-8-(trifluoromethyl)-7,8-dihydro-6H-pyrazolo[1,5-a]pyrrolo[2,3-e]pyrimidine-6-carboxamide

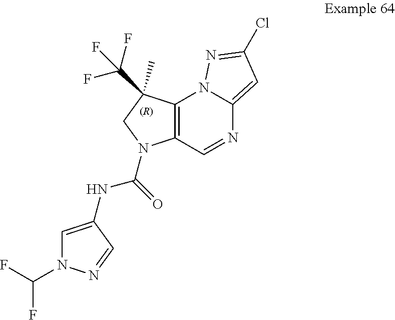

The title compound was prepared according to Method M1 Step 2 by using 1-(difluoromethyl)-1H-pyrazol-4-amine and Method M1 Isomer 2. The enantiomer of Example 64 can be prepared analogously using Method M1 isomer 1.

Example 64: ¹H NMR (300 MHz, DMSO-d₆) δ: 9.46 (s, 1H), 9.36 (s, 1H), 8.28 (s, 1H), 7.85 (s, 1H), 7.79 (t, J=58.8 Hz, 1H), 7.05 (s, 1H), 4.70 (d, J=11.4 Hz, 1H), 4.20 (d, J=11.4 Hz, 1H), 1.97 (s, 3H). LC-MS: m/z 436 [M+H]⁺

### Method Q2

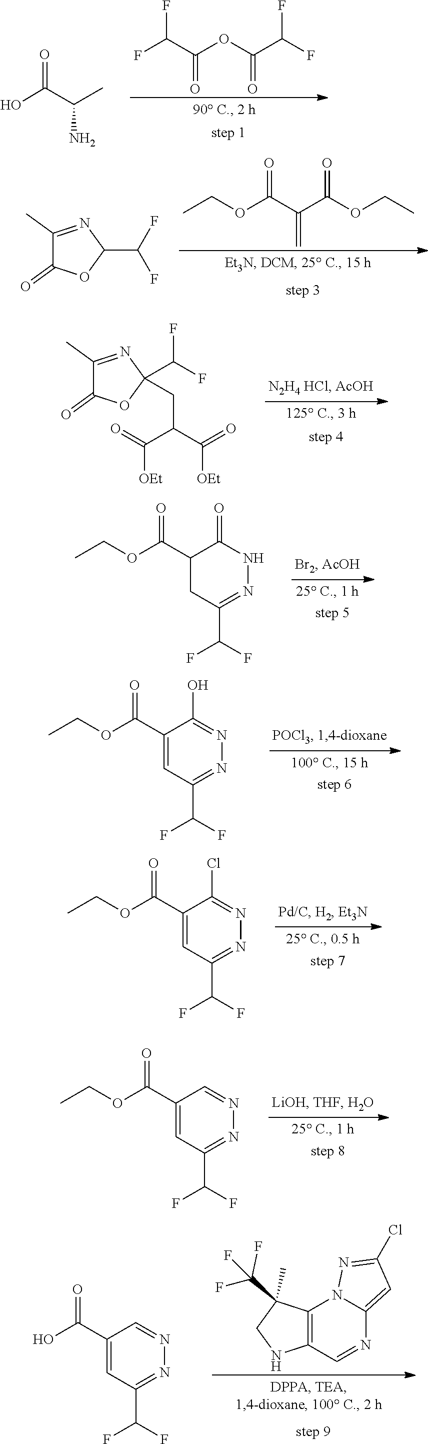

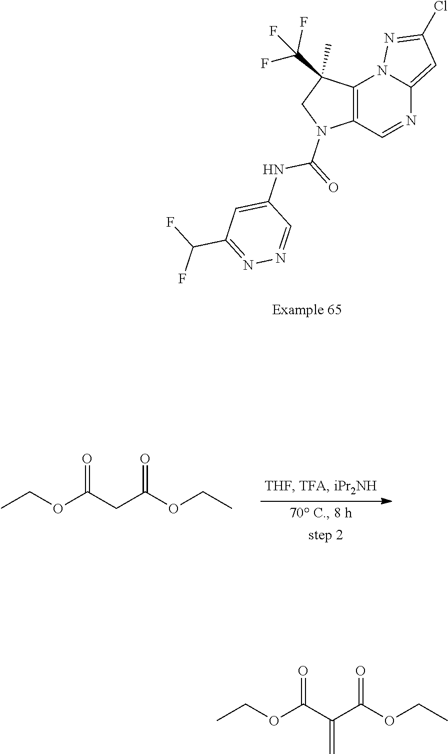 [SMILES: C[C@@]1(C(F)(F)F)CN(C(=O)Nc2cnnc(C(F)F)c2)c2cnc3cc(Cl)nn3c21 ; CCOC(=O)CC(=O)OCC ; C=C(C(=O)OCC)C(=O)OCC]

### Example 65: (R)-2-choro-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide

### Step 1: 2-(difluoromethyl)-4-methyloxazol-5(2H)-one

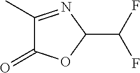

2,2-difluoroacetic anhydride (62.5 g, 359.2 mmol) was added slowly over 10 min to L-alanine (12.5 g, 140.4 mmol) at 0° C. After 0.5 h of additional stirring, the clear mixture was stirred for 2 h at 90° C. The reaction mixture was concentrated under vacuum. The residue was diluted with dichloromethane (200 mL), and the pH was adjusted to 7-8 with saturated aqueous NaHCO₃ solution. The resulting solution was extracted with dichloromethane (2×500 mL). The organic layers were combined, dried over anhydrous sodium sulfate and concentrated under vacuum to give 2-(difluoromethyl)-4-methyloxazol-5(2H)-one (7.1 g, crude) as a black oil. The product was used in the next step directly without further purification. ¹H NMR (400 MHz, Chloroform-d) δ: 5.80-6.09 (m, 2H), 2.35 (d, J=2.3 Hz, 3H). LC-MS: m/z 150 [M+H]⁺.

### Step 2: diethyl 2-methylenemalonate

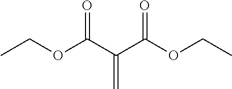

A flame-dried 500 mL round-bottom flask was charged with dry tetrahydrofuran (150 mL), diisopropylamine (10.1 g, 99.9 mmol), TFA (12.5 g, 109.9 mmol), diethyl malonate (16.0 g, 99.9 mmol) and paraformaldehyde (6.0 g, 199.8 mmol). A condenser was added and the suspension was stirred for 2 h at 70° C. A second batch of paraformaldehyde (6.0 g, 199.8 mmol) was added at 70° C. for another 6 h. The reaction was concentrated under vacuum. The crude mixture was dissolved in diethyl ether (80 mL). The solid was filtered out. The filtrate was washed with 1 N aqueous hydrochloric acid solution (2×80 mL). The aqueous layers were combined and extracted with diethyl ether (80 mL). The organic layers were combined, dried over anhydrous sodium sulfate and concentrated under vacuum to give diethyl 2-methylenepropanedioate (19 g, crude) as a yellow liquid. ¹H NMR (300 MHz, Chloroform-d) δ: 6.52 (s, 2H), 4.14-4.30 (m, 4H), 1.18-1.34 (m, 6H). LC-MS: m/z 173 [M+H]⁺.

### Step 3: diethyl 2-((2-(difluoromethyl)-4-methyl-5-oxo-2,5-dihydrooxazol-2-yl)methyl)malonate

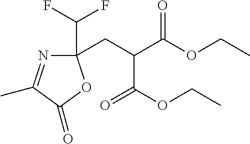 [SMILES: CCOC(=O)C(CC1(C(F)F)N=C(C)C(=O)O1)C(=O)OCC]

To a stirred solution of 2-(difluoromethyl)-4-methyloxazol-5(2H)-one (7.1 g, crude) in dichloromethane (70 mL) were added diethyl 2-methylenepropanedioate (9.8 g, 57.2 mmol) and TEA (7.2 g, 71.5 mmol) at 0° C. The reaction mixture was stirred at h 23° C. for 15. The reaction mixture was diluted with water (80 mL) and extracted with dichloromethane (2×80 mL). The organic layers were combined, dried over anhydrous sodium sulfate and concentrated under vacuum to give diethyl 2-((2-(difluoromethyl)-4-methyl-5-oxo-2,5-dihydrooxazol-2-yl)methyl)malonate (12.4 g, crude) as a black oil. The crude material was used in the next step without further purification. LC-MS: m/z 322 [M+H]⁺.

### Step 4: ethyl 6-(difluoromethyl)-3-oxo-2,3,4,5-tetrahydropyridazine-4-carboxylate

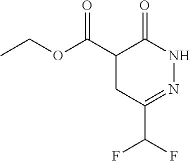

To a stirred solution of diethyl 2-((2-(difluoromethyl)-4-methyl-5-oxo-2,5-dihydrooxazol-2-yl)methyl)malonate (12.4 g, crude) in acetic acid (150 mL) was added dihydrogen hydrochloride (13.8 g, 201.8 mmol) at 23° C. The reaction mixture was stirred at 125° C. for 3 h. The reaction mixture was concentrated under vacuum. The residue was diluted with ethyl acetate (200 mL) and the pH was adjusted to 7-8 with saturated aqueous NaHCO₃ solution. The resulting solution was extracted with ethyl acetate (3×300 mL). The organic layers were combined, dried over anhydrous sodium sulfate and concentrated under vacuum. The residue was purified by column chromatography on silica gel using 65% petroleum ether and 35% ethyl acetate as eluent to afford ethyl 6-(difluoromethyl)-3-oxo-2,3,4,5-tetrahydropyridazine-4-carboxylate (2.9 g, 9% yield) as a yellow oil. ¹H NMR (300 MHz, Chloroform-d) δ: 8.89-8.96 (m, 1H), 5.96-6.32 (m, 1H), 4.25-4.34 (m, 2H), 3.58 (t, J=8.0 Hz, 1H), 2.48-2.95 (m, 2H), 1.30-1.37 (m, 3H). LC-MS: m/z 221 [M+H]⁺.

### Step 5: ethyl 6-(difluoromethyl)-3-hydroxypyridazine-4-carboxylate

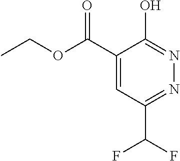

To a stirred solution of ethyl 6-(difluoromethyl)-3-oxo-2,3,4,5-tetrahydropyridazine-4-carboxylate (2.9 g, 13.2 mmol) in acetic acid (25 mL) was added a solution of bromine (2.1 g, 13.2 mmol) in acetic acid (15 mL) at 0° C. The reaction mixture was stirred at 23° C. for 1 h. The reaction mixture was concentrated under vacuum. The residue was diluted with ethyl acetate (100 mL) and the pH was adjusted to 7-8 with saturated aqueous NaHCO₃ solution. The resulting solution was extracted with ethyl acetate (2×200 mL). The organic layers were combined, dried over anhydrous sodium sulfate and concentrated under vacuum. The residue was purified by column chromatography on silica gel using 60% petroleum ether and 40% ethyl acetate as eluent to afford ethyl 6-(difluoromethyl)-3-hydroxypyridazine-4-carboxylate (1.6 g, 34% yield) as a light yellow solid. ¹H NMR (400 MHz, DMSO-d₆) δ: 13.89 (s, 1H), 7.98 (s, 1H), 6.78-7.04 (m, 1H), 4.27 (q, J=7.1 Hz, 2H), 1.27 (t, J=7.1 Hz, 3H). LC-MS: m/z 219 [M+H]⁺.

### Step 6: ethyl 3-chloro-6-(difluoromethyl)pyridazine-4-carboxylate

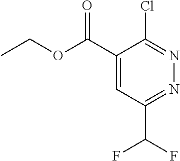

To a stirred solution of ethyl 6-(difluoromethyl-3-hydroxypyridazine-4-carboxylate (1.6 g, 7.3 mmol) in dioxane (20 mL) was added phosphoryl trichloride (11.3 g, 73.3 mmol) at 0° C. The reaction mixture was stirred at 100° C. for 15 h. The reaction mixture was concentrated under vacuum. The residue was diluted with ethyl acetate (100 mL) and the pH was adjusted to 7-8 with saturated aqueous NaHCO₃ solution. The resulting solution was extracted with ethyl acetate (2×200 mL). The organic layers were combined, dried over anhydrous sodium sulfate and concentrated under vacuum. The residue was purified by column chromatography on silica gel using 84% petroleum ether and 16% ethyl acetate as eluent to afford ethyl 3-chloro-6-(difluoromethyl)pyridazine-4-carboxylate (1.4 g, 82% yield) as a yellow liquid. ¹H NMR (300 MHz, DMSO-d₆) δ: 8.42 (s, 1H), 7.20-7.56 (m, 1H), 4.42 (q, J=7.1 Hz, 2H), 1.36 (t, J=7.1 Hz, 3H). LC-MS: m/z 237 [M+H]⁺.

### Step 7: ethyl 6-(difluoromethyl)pyridazine-4-carboxylate

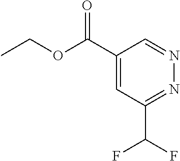

To a stirred solution of ethyl 3-chloro-6-(difluoromethyl)pyridazine-4-carboxylate (600 mg, 2.5 mmol) in ethyl acetate (10 mL) was added TEA (0.6 mL) and Pd/C (120 mg, 10%) at 23° C. The reaction mixture was stirred at 23° C. under a hydrogen atmosphere (balloon pressure) for 0.5 h. The solid was filtered out. The filtrate was concentrated under vacuum. The residue was purified by column chromatography on silica gel using 90% petroleum ether and 10% ethyl acetate as eluent to afford ethyl 6-(difluoromethyl)pyridazine-4-carboxylate (230 mg, 44% yield) as a yellow liquid. ¹H NMR (300 MHz, DMSO-d₆) δ: 9.75 (d, J=1.9 Hz, 1H), 8.29 (d, J=2.0 Hz, 1H), 7.23-7.59 (m, 1H), 4.43 (q, J=7.1 Hz, 2H), 1.38 (t, J=7.1 Hz, 3H). LC-MS: m/z 203 [M+H]⁺.

### Step 8: 6-(difluoromethyl)pyridazine-4-carboxylic acid

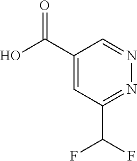 [SMILES: O=C(O)c1cnnc(C(F)F)c1]

To a stirred solution of ethyl 6-(difluoromethyl)pyridazine-4-carboxylate (230 mg, 1.1 mmol) in a mixture of tetrahydrofuran (6 mL) and water (1.5 mL) was added lithium hydroxide (81.7 mg, 3.4 mmol) at 0° C. The reaction mixture was gradually warmed to 23° C. and stirred for 1 h. The mixture was concentrated under reduced pressure then diluted with water (30 mL). The pH was adjusted to 2-3 with HCl (1 N) and extracted with ethyl acetate (3×35 mL). The organic layers were combined, dried over anhydrous sodium sulfate and concentrated under vacuum. The obtained solid was washed with n-pentane (20 mL) to afford 6-(difluoromethyl)pyridazine-4-carboxylic acid (160 mg, 74% yield) as an off white solid. ¹H NMR (400 MHz, DMSO-d₆) δ: 9.68 (d, J=1.9 Hz, 1H), 8.21 (d, J=1.9 Hz, 1H), 7.23-7.50 (m, 1H). LC-MS: m/z 175 [M+H]⁺.

### Step 9: (R)-2-chloro-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide

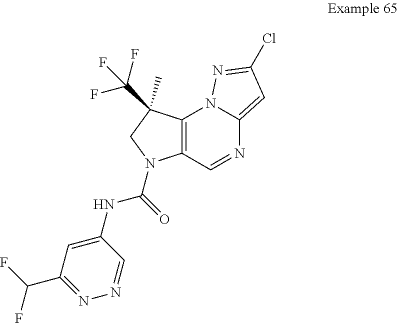 [SMILES: C[C@@]1(C(F)(F)F)CN(C(=O)Nc2cnnc(C(F)F)c2)c2cnc3cc(Cl)nn3c21]

To a stirred solution of 6-(difluoromethyl)pyridazine-4-carboxylic acid (60 mg, 344.8 umol) in dioxane (8 mL) was added Method M1 isomer 2 (95.2 mg, 344.8 μmol), TEA (174.1 mg, 1.7 mmol) and DPPA (106.3 mg, 413.8 μmol). The mixture was stirred at 100° C. for 2 h. The reaction mixture was concentrated under vacuum. The residue was purified by Prep-TLC using 50% petroleum ether and 50% ethyl acetate as eluent to afford 110 mg of crude product. The afforded crude was purified by Prep-HPLC purification and the collected fractions were lyophilized to give (R)-2-chloro-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide (31.8 mg, 20.6% yield) as a white solid. The enantiomer of Example 65 can be prepared analogously using Method M1 isomer 1.

Example 65: ¹H NMR (400 MHz, DMSO-d₆) δ: 9.93 (s, 1H), 9.51 (d, J=2.5 Hz, 1H), 9.35 (s, 1H), 8.21 (d, J=2.5 Hz, 1H), 7.07-7.40 (m, 2H), 4.88 (d, J=11.6 Hz, 1H), 4.31 (d, J=11.6 Hz, 1H), 1.98 (s, 3H). LC-MS: m/z 448 [M+H]⁺.

### Method R2

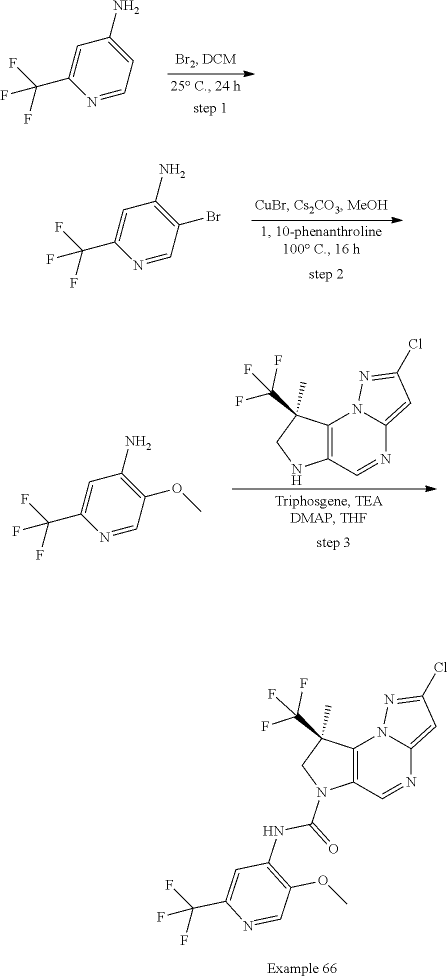 [SMILES: Nc1ccnc(C(F)(F)F)c1 ; Nc1cc(C(F)(F)F)ncc1Br ; COc1cnc(C(F)(F)F)cc1N ; C[C@@]1(C(F)(F)F)CNc2cnc3cc(Cl)nn3c21 ; COc1cnc(C(F)(F)F)cc1NC(=O)N1C[C@@](C)(C(F)(F)F)c2c1cnc1cc(Cl)nn21]

### Examples 66: (R)-2-chloro-N-(5-methoxy-2-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

### Step 1: 5-bromo-2-(trifluoromethyl)pyridin-4-amine

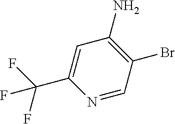 [SMILES: Nc1cc(C(F)(F)F)ncc1Br]

To a solution of 2-(trifluoromethyl)pyridin-4-amine (3.0 g, 18.5 mmol) in dichloromethane (50 mL) was added a solution of bromine (3.0 g, 18.5 mmol) in dichloromethane (50 mL) dropwise at 0° C. The resulting mixture was stirred at 25° C. for 24 h. The organic solution was washed with saturated aqueous NH₄Cl solution (100 mL), water (50 mL), dried over anhydrous sodium sulfate and concentrated under vacuum to afford 5-bromo-2-(trifluoromethyl)pyridin-4-amine (4.0 g, 81% yield) as a yellow solid. ¹H NMR (400 MHz, DMSO-d₆) δ: 8.40 (s, 1H), 7.10 (s, 1H), 6.88 (s, 2H). LC-MS: m/z 241 [M+H]⁺.

### Step 2: 5-methoxy-2-(trifluoromethyl)pyridin-4-amine

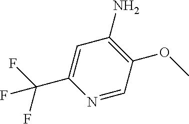

To a mixture of 5-bromo-2-(trifluoromethyl)pyridin-4-amine (200 mg, 829.8 μmol) in methanol (5 mL) were added copper(I) bromide (71.4 mg, 497.9 μmol), Cs₂CO₃ (541 mg, 1.7 mmol) and 1,10-phenanthroline (45 mg, 248.9 μmol) under nitrogen atmosphere. The resulting mixture was stirred at 100° C. for 16 h. The solid was filtered out. The filtrate was concentrated under vacuum. The residue was purified by column chromatography on silica gel using 75% petroleum ether and 25% ethyl acetate as eluent to afford 5-methoxy-2-(trifluoromethyl)pyridin-4-amine (60 mg, 37% yield) as a pink solid. ¹H NMR (400 MHz, DMSO-d₆) δ: 7.99 (s, 1H), 6.96 (s, 1H), 6.20 (s, 2H), 3.90 (s, 3H). LC-MS: m/z 193 [M+H]⁺.

### Step 3: (R)-2-chloro-N-(5-methoxy-2-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

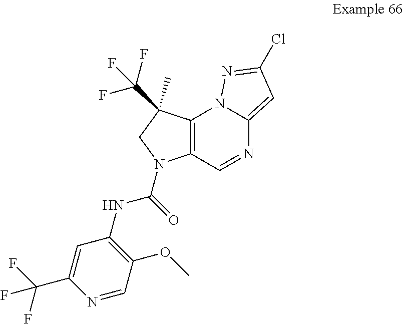 [SMILES: COc1cnc(C(F)(F)F)cc1NC(=O)N1C[C@@](C)(C(F)(F)F)c2c1cnc1cc(Cl)nn21]

To a stirred solution of Method M1 isomer 2 (20 mg, 72.3 μmol) in tetrahydrofuran (2 mL) were added triphosgene (13 mg, 43.4 μmol) and TEA (11 mg, 108.4 μmol). The resulting mixture was stirred at 25° C. for 0.5 h and then filtered. The filtrate was added to a solution of 5-methoxy-2-(trifluoromethyl)pyridin-4-amine (25 mg, 130.1 μmol) in tetrahydrofuran (1 mL). To this solution were added N,N-dimethylpyridin-4-amine (18 mg, 144.6 μmol) and TEA (73 mg, 723.0 μmol). The mixture was stirred at 40° C. for 1 h. The mixture was concentrated under vacuum. The residue was purified by Prep-HPLC and the collected fractions were lyophilized to afford (R)-2-chloro-N-(5-methoxy-2-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide (20.4 mg, 57% yield) as a white solid. The enantiomer of Example 66 can be prepared analogously using Method M1 isomer 1.

Example 66: ¹H NMR (400 MHz, DMSO-d₆) δ: 9.31 (s, 1H), 8.86 (s, 1H), 8.52 (s, 1H), 8.40 (s, 1H), 7.07 (s, 1H), 4.96 (d, J=11.2 Hz, 1H), 4.32 (d, J=11.6 Hz, 1H), 4.09 (s, 3H), 1.96 (s, 3H). LC-MS: m/z 495 [M+H]⁺.

### Method S2

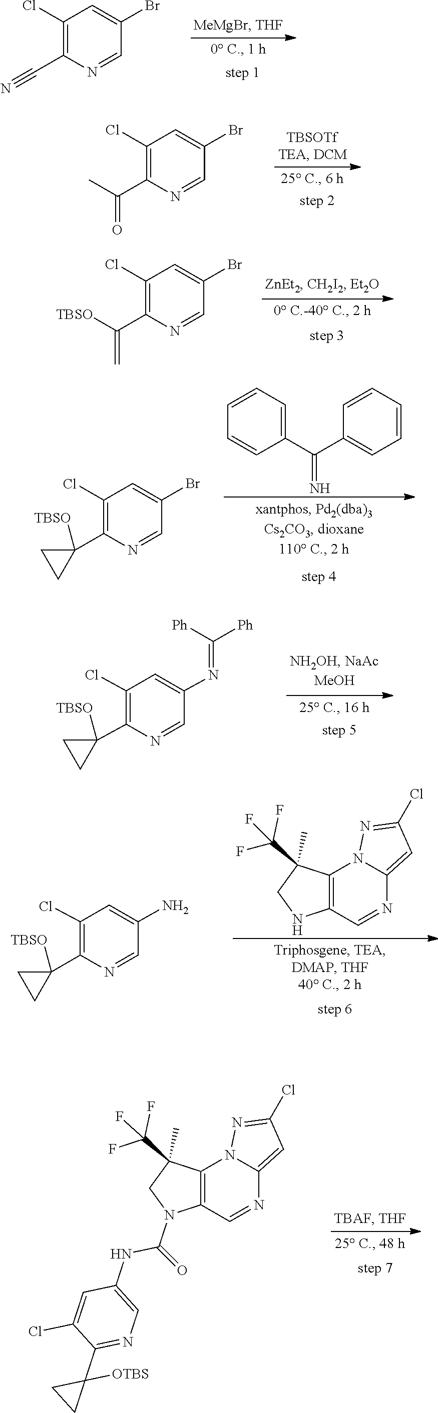

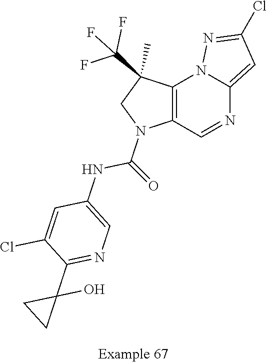 [SMILES: C[C@@]1(C(F)(F)F)CN(C(=O)Nc2cnc(C3(O)CC3)c(Cl)c2)c2cnc3cc(Cl)nn3c21]

### Examples 67: (R)-2-chloro-N-(5-chloro-6-(1-hydroxycyclopropyl)pyridin-3-yl)-8-methyl-(trifluormethyl)-7,8-dihydro-6H-pyrazolo[1,5-a]pyrrolo[2,3-e]pyrimidine-6-carboxamide

### Step 1: 1-(5-bromo-3-chloropyridin-2-yl)ethan-1-one

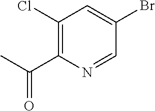

To a stirred solution of 5-bromo-3-chloro-pyridine-2-carbonitrile (15.0 g, 68.9 mmol) in tetrahydrofuran (150 mL) was added methylmagnesium bromide (34.8 mL, 308.4 mmol, 1M in tetrahydrofuran) dropwise at 0° C. The mixture was stirred at 0° C. for 1 h. The reaction mixture was quenched by the addition of saturated aqueous NH₄Cl solution (200 mL) at 0° C. The resulting mixture was extracted with ethyl acetate (3×200 mL). The combined organic layers were dried over anhydrous sodium sulfate and concentrated under vacuum. The residue was purified by column chromatography on silica gel using 50% petroleum ether and 50% ethyl acetate as eluent to afford 1-(5-bromo-3-chloropyridin-2-yl)ethan-1-one (6 g, 37% yield) as a yellow solid. ¹H NMR (400 MHz, Chloroform-d) δ 8.59 (d, J=2.0 Hz, 1H), 7.97 (d, J=2.0 Hz, 1H), 2.66 (s, 3H). LC-MS: m/z 234 [M+H]⁺.

### Step 2: 5-bromo-2-(1-((tert-butyldimethylsilyl)oxy)vinyl)-3-chloropyridine

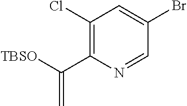 [SMILES: C=C(O[Si](C)(C)C(C)(C)C)c1ncc(Br)cc1Cl]

To a stirred solution of 1-(5-bromo-3-chloropyridin-2-yl)ethan-1-one (2.0 g, 8.4 mmol) indichloromethane (100 mL) were added TEA (2.5 g, 25.3 mmol) and tert-Butyldimethylsilyl trifluoromethanesulfonate (2.9 g, 10.9 mmol). The mixture was stirred at 25° C. for 6 h. The resulting mixture was diluted with water (100 mL). The resulting mixture was extracted with dichloromethane (3×200 mL). The combined organic layers were dried over anhydrous sodium sulfate and concentrated under vacuum. The residue was purified by column chromatography on silica gel using 90% petroleum ether and 10% ethyl acetate as eluent to afford 5-bromo-2-(1-((tert-butyldimethylsilyl)oxy)vinyl)-3-chloropyridine (2.5 g, 84% yield) as a yellow solid. ¹H NMR (300 MHz, Chloroform-d) δ 8.52 (d, J=2.1 Hz, 1H), 7.87 (d, J=2.1 Hz, 1H), 4.82 (d, J=1.8 Hz, 1H), 4.77 (d, J=1.8 Hz, 1H), 0.92 (s, 9H), 0.17 (s, 6H). LC-MS: m/z 348 [M+H]⁺.

### Step 3: 5-bromo-2-(1-((tert-butyldimethylsilyl)oxy)cyclopropyl)-3-chloropyridine

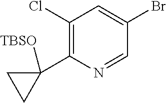 [SMILES: CC(C)(C)[Si](C)(C)OC1(c2ncc(Br)cc2Cl)CC1]

To a stirred solution of 5-bromo-2-(1-((tert-butyldimethylsilyl)oxy)vinyl)-3-chloropyridine (1.0 g, 2.8 mmol) and diethylzinc (8.5 mL, 8.6 mmol, 1 M in hexane) in diethyl ether (16 mL) was added dropwise a solution of diiodomethane (2.4 g, 8.9 mmol) in diethyl ether (16 mL at 0° C.) over 1 h. The mixture was stirred at 40° C. for 2 h. The reaction was quenched by the addition of methanol (50 mL). The solid was filtered out. The filtrate was concentrated under vacuum. The residue was purified by column chromatography on silica gel using 90% petroleum ether and 10% ethyl acetate as eluent to afford to afford 5-bromo-2-(1-((tert-butyldimethylsilyl)oxy)cyclopropyl)-3-chloropyridine (280 mg, 27% yield) as yellow oil. ¹H NMR (400 MHz, Chloroform-d) δ 8.47 (d, J=2.0 Hz, 1H), 7.89 (d, J=2.0 Hz, 1H), 1.11-1.14 (m, 4H), 0.78 (s, 9H), 0.10 (s, 6H). LC-MS: m/z 362 [M+H]⁺.

### Step 4: N-(6-(1-((tert-butyldimethylsilyl)oxy)cyclopropyl)-5-chloropyridin-3-yl)-1,1-diphenylmethanimine

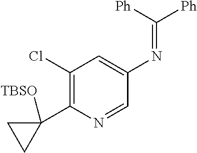 [SMILES: CC(C)(C)[Si](C)(C)OC1(c2ncc(N=C(c3ccccc3)c3ccccc3)cc2Cl)CC1]

To a stirred solution of 5-bromo-2-(1-((tert-butyldimethylsilyl)oxy)cyclopropyl)-3-chloropyridine (300 mg, 826.9 μmol) and diphenylmethanimine (80 mg, 992.3 μmol) in dioxane (4 mL) were added Xantphos (143 mg, 248.0 μmol), Pd₂(dba)₃ (95 mg, 165.4 μmol) and Cs₂CO₃ (808 mg, 2.4 mmol) under nitrogen atmosphere. The resulting mixture was stirred at 110° C. for 2 h. The solid was filtered out. The filtrate was concentrated under vacuum. The residue was purified by column chromatography on silica gel using 90% petroleum ether and 10% ethyl acetate as eluent to afford N-(6-(1-((tert-butyldimethylsilyl)oxy)cyclopropyl)-5-chloropyridin-3-yl)-1,1-diphenylmethanimine (170 mg, 44% yield) as a yellow oil. ¹H NMR (300 MHz, Chloroform-d) δ 7.72-7.83 (m, 4H), 7.38-7.54 (m, 5H), 7.06-7.14 (m, 3H), 1.01-1.07 (m, 4H), 0.77 (s, 9H), 0.21 (s, 6H). LC-MS: m/z 463 [M+H]⁺.

### Step 5: 6-(1-((tert-butyldimethylsilyl)oxy)cyclopropyl)-5-chloropyridin-3-amine

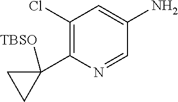 [SMILES: CC(C)(C)[Si](C)(C)OC1(c2ncc(N)cc2Cl)CC1]

Into a 50 mL round bottom flask were placed N-(6-(1-((tert-butyldimethylsilyl)oxy)cyclopropyl)-5-chloropyridin-3-yl)-1,1-diphenylmethanimine (200 mg, 431.8 μmol), hydroxylamine hydrochloride (60 mg, 863.4 μmol), sodium acetate (141 mg, 1.7 mmol) and methanol (10 mL). The mixture was stirred at 25° C. for 16 h. The mixture was concentrated under vacuum. The residue was purified by column chromatography on silica gel using 50% petroleum ether and 50% ethyl acetate as eluent to afford 6-(1-((tert-butyldimethylsilyl)oxy)cyclopropyl)-5-chloropyridin-3-amine (70 mg, 54% yield) as a yellow solid. ¹H NMR (400 MHz, Chloroform-d) δ 7.92 (s, 1H), 7.04 (s, 1H), 3.87 (s, 2H), 1.03-1.11 (m, 4H), 0.78 (s, 9H), 0.10 (s, 6H). LC-MS: m/z 299 [M+H]⁺.

### Step 6: (R)—N-(6-(1-((tert-butyldimethylsilyl)oxy)cyclopropyl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide

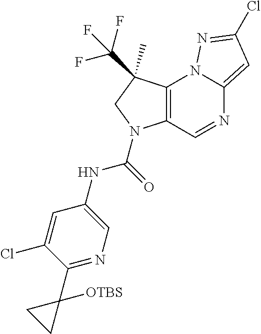 [SMILES: CC(C)(C)[Si](C)(C)OC1(c2ncc(NC(=O)N3C[C@@](C)(C(F)(F)F)c4c3cnc3cc(Cl)nn43)cc2Cl)CC1]

To a stirred solution of Method M1 isomer 2 (17 mg, 61.4 μmol) in tetrahydrofuran (4 mL) were added triphosgene (10 mg, 36.8 μmol) and TEA (9 mg, 92.1 μmol) at 0° C. The resulting mixture was stirred at 25° C. for 0.5 h and then filtered. The filtrate was added to a solution of 6-(1-((tert-butyldimethylsilyl)oxy)cyclopropyl)-5-chloropyridin-3-amine (18 mg, 61.4 μmol) in tetrahydrofuran (2 mL). To this solution was then added N,N-dimethylpyridin-4-amine (15 mg, 122.9 μmol) and TEA (62 mg, 614.5 μmol). The mixture was stirred at 40° C. for 2 h. The mixture was poured into water (10 mL) and extracted with ethyl acetate (3×10 mL). The combined organic layers were washed with brine (20 mL), dried over anhydrous sodium sulfate and concentrated under vacuum. The residue was purified by Prep-TLC using 95% dichloromethane and 5% methanol as eluent to afford (R)—N-(6-(1-((tert-butyldimethylsilyl)oxy)cyclopropyl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide (16 mg, 43% yield) as a yellow oil. ¹H NMR (400 MHz, DMSO-d6) δ 9.37 (s, 1H), 9.32 (s, 1H), 8.59 (d, J=2.4 Hz, 1H), 8.17 (d, J=2.4 Hz, 1H), 7.04 (s, 1H), 4.79 (d, J=11.6 Hz, 1H), 4.24 (d, J=11.6 Hz, 1H), 1.94 (s, 3H), 1.00-1.06 (m, 2H), 0.80-0.86 (m, 2H), 0.74 (s, 9H), 0.15 (s, 6H). LC-MS: m/z 601 [M+H]⁺

### Step 7: (R)-2-chloro-N-(5-chloro-6-(1-hydroxycyclopropyl)pyridin-3-yl)-8-methyl-8-(trifluoromethyl)-7,8-dihydro-6H-pyrazolo[1,5-a]pyrrolo[2,3-e]pyrimidine-6-carboxamide

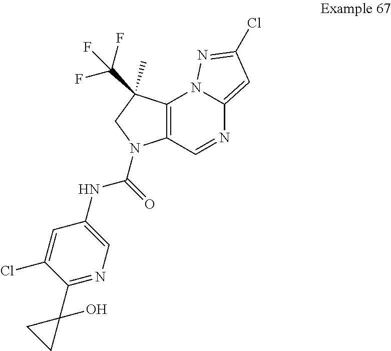 [SMILES: C[C@@]1(C(F)(F)F)CN(C(=O)Nc2cnc(C3(O)CC3)c(Cl)c2)c2cnc3cc(Cl)nn3c21]

To a stirred solution of (R)—N-(6-(1-((tert-butyldimethylsilyl)oxy)cyclopropyl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide (10 mg, 16.6 μmol) in tetrahydrofuran (2 mL) was added TBAF (166 uL, 166 μmol, 1 M in tetrahydrofuran) at 25° C. The resulting mixture was stirred at 25° C. for 48 h. The resulting mixture was concentrated under vacuum. The residue was purified by Prep-TLC using ethyl acetate as eluent to afford crude product (20 mg). The residue was purified by Prep-HPLC and the collected fractions were lyophilized to give (R)-2-chloro-N-(5-chloro-6-(1-hydroxycyclopropyl)pyridin-3-yl)-8-methyl-8-(trifluoromethyl)-7,8-dihydro-6H-pyrazolo[1,5-a]pyrrolo[2,3-e]pyrimidine-6-carboxamide (1.9 mg, 23% yield) as a white solid. The enantiomer of Example 67 can be prepared analogously using Method M1 isomer 1.

Example 67: ¹H NMR (400 MHz, DMSO-d₆) δ 9.37 (s, 1H), 9.33 (s, 1H), 8.58 (d, J=2.0 Hz, 1H), 8.15 (d, J=2.0 Hz, 1H), 7.06 (s, 1H), 5.95 (s, 1H), 4.81 (d, J=11.6 Hz, 1H), 4.25 (d, J=11.6 Hz, 1H), 1.97 (s, 3H), 0.96-1.02 (m, 4H). LC-MS: m/z 487 [M+H]⁺

### Method T2

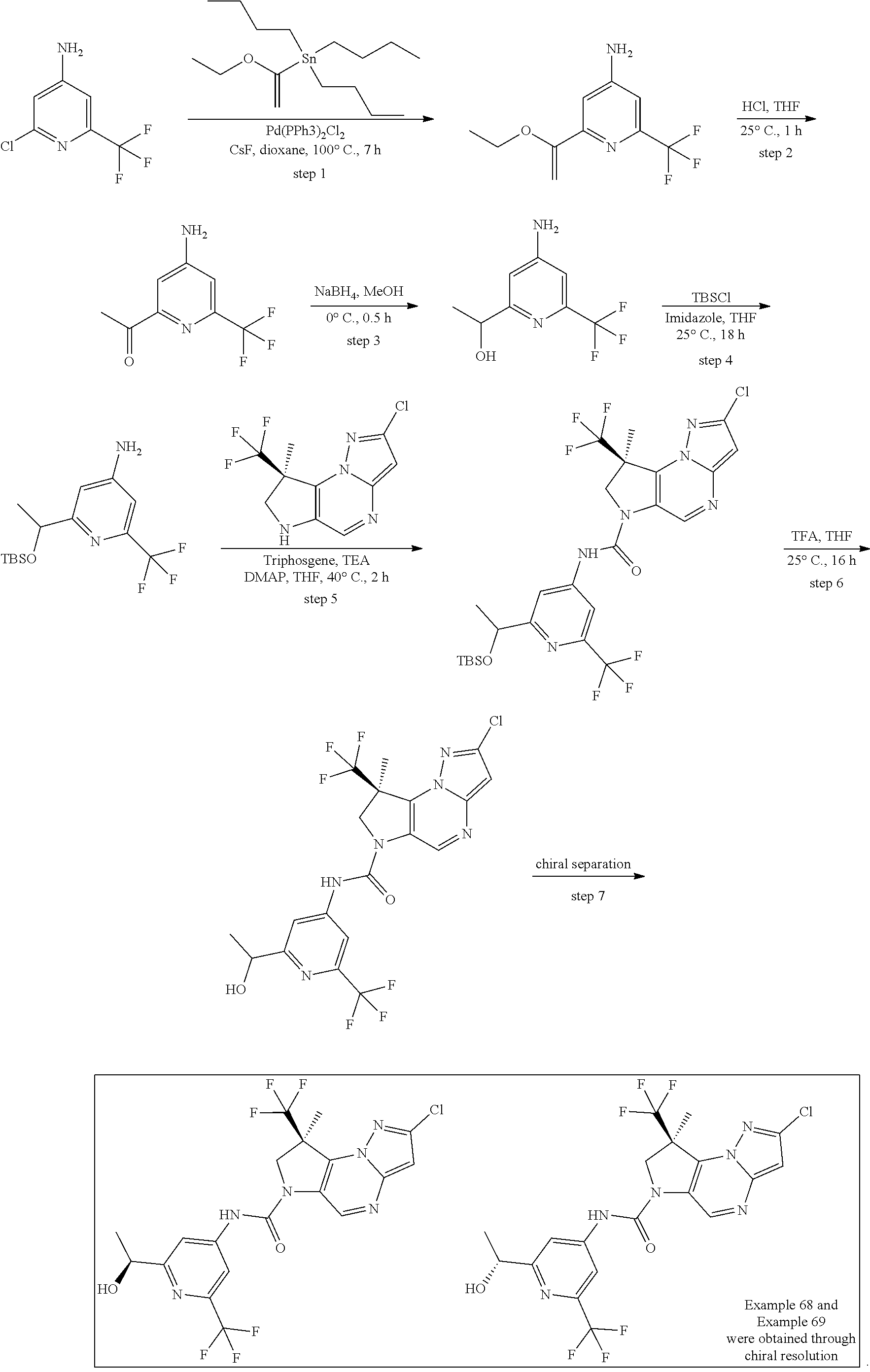

### Examples 68 and 69: Single enantiomers obtained from a racemic mixture containing (R)-2-chloro-N-(2-((S)-1-hydroxyethyl)-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(2-((R)-1-hydroxyethyl)-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

### Step 1: 2-(1-ethoxyvinyl)-6-(trifluoromethyl)pyridin-4-amine

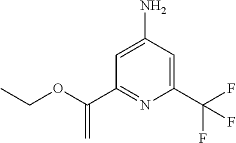 [SMILES: C=C(OCC)c1cc(N)cc(C(F)(F)F)n1]

To a mixture of 2-chloro-6-(trifluoromethyl)pyridin-4-amine (1 g, 5.1 mmol) in Dioxane (10 mL) were added tributyl(1-ethoxyvinyl)stannane (2.2 g, 6.1 mmol), CsF (1.7 g, 11.2 mmol) and Pd(PPh₃)₂Cl₂ (178 mg, 254.4 μmol) under nitrogen atmosphere. The resulting mixture was stirred at 100° C. for 7 h. The reaction mixture was quenched with water (100 mL). The resulting solution was extracted with ethyl acetate (3×100 mL). The combined organic layers were dried over anhydrous sodium sulfate and concentrated under vacuum. The residue was purified by column chromatography on silica gel using 75% petroleum ether and 25% ethyl acetate as eluent to afford 2-(1-ethoxyvinyl)-6-(trifluoromethyl)pyridin-4-amine (0.8 g, 68% yield) as a yellow solid. LC-MS: m/z 233 [M+H]⁺.

### Step 2: 1-(4-amino-6-(trifluoromethyl)pyridin-2-yl)ethan-1-one

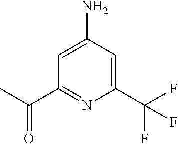

To a solution of 2-(1-ethoxyvinyl)-6-(trifluoromethyl)pyridin-4-amine (700 mg, 3.0 mmol) in tetrahydrofuran (15 mL) was added HCl(aq) (7.5 mL, 1 M) at 0° C. The reaction mixture was stirred at 25° C. for 1 h. The pH was adjusted to 7 with saturated aqueous NaHCO₃ solution. The resulting solution was extracted with ethyl acetate (3×50 mL). The combined organic solution was washed with brine (150 mL), dried over anhydrous sodium sulfate and concentrated under vacuum to afford 1-(4-amino-6-(trifluoromethyl)pyridin-2-yl)ethan-1-one (600 mg, 92% yield) as a yellow solid. ¹H NMR (400 MHz, Chloroform-d) δ: 7.37 (d, J=2.4 Hz, 1H), 7.02 (d, J=2.4 Hz, 1H), 4.60 (s, 2H), 2.70 (s, 3H). LC-MS: m/z 205 [M+H]⁺.

### Step 3: 1-(4-amino-6-(trifluoromethyl)pyridin-2-yl)ethan-1-ol

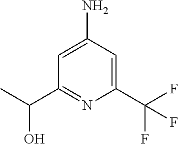

To a solution of 1-(4-amino-6-(trifluoromethyl)pyridin-2-yl)ethan-1-one (600 mg, 2.9 mmol) in methanol (10 mL) was added sodium tetrahydroborate (133 mg, 3.5 mmol) at 0° C. The reaction mixture was stirred at 0° C. for 1 h. The reaction mixture was concentrated under vacuum. Water (10 mL) was added and the resulting mixture was extracted with ethyl acetate (3×20 mL). The combined organic layers were dried over anhydrous sodium sulfate and concentrated under vacuum. The residue was purified by column chromatography on silica gel using 50% petroleum ether and 50% ethyl acetate to afford 1-(4-amino-6-(trifluoromethyl)pyridin-2-yl)ethan-1-ol (450 mg, 74% yield) as a white solid. ¹H NMR (300 MHz, Methanol-d₄) δ: 6.87 (d, J=2.1 Hz, 1H), 6.80 (d, J=2.1 Hz, 1H), 4.71 (q, J=6.6 Hz, 1H), 1.40 (d, J=6.6 Hz, 3H). LC-MS: m/z 207 [M+H]⁺.

### Step 4: 2-(1-((tert-butyldimethylsilyl)oxy)ethyl)-6-(trifluoromethyl)pyridin-4-amine

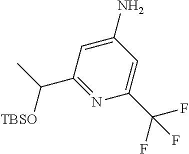 [SMILES: CC(O[Si](C)(C)C(C)(C)C)c1cc(N)cc(C(F)(F)F)n1]

To a solution of 1-(4-amino-6-(trifluoromethyl)pyridin-2-yl)ethan-1-ol (450 mg, 2.2 mmol) in tetrahydrofuran (10 mL) were added imidazole (446 mg, 6.5 mmol) and tert-butylchlorodimethylsilane (395 mg, 2.6 mmol). The reaction mixture was stirred at 25° C. for 18 h. The mixture was concentrated under vacuum. The residue was purified by column chromatography on silica gel using 75% petroleum ether and 25% ethyl acetate as eluent to afford 2-(1-((tert-butyldimethylsilyl)oxy)ethyl)-6-(trifluoromethyl)pyridin-4-amine (500 mg, 71% yield) as an off-white solid. ¹H NMR (400 MHz, DMSO-d₆) δ: 6.80 (d, J=2.0 Hz, 1H), 6.75 (d, J=2.4 Hz, 1H), 6.58 (s, 2H), 4.71 (q, J=6.4 Hz, 1H), 1.31 (d, J=6.4 Hz, 3H), 0.89 (s, 9H), 0.06 (s, 3H), 0.02 (s, 3H). LC-MS: m/z 321 [M+H]⁺.

### Step 5: (8R)—N-(2-(1-((tert-butyldimethylsilyl)oxy)ethyl)-6-(trifluoromethyl)pyridin-4-yl)-2-chloro-8-methyl-8-(trifluoromethyl)-7,8-dihydro-6H-pyrazolo[1,5-a]pyrrolo[2,3-e]pyrimidine-6-carboxamide

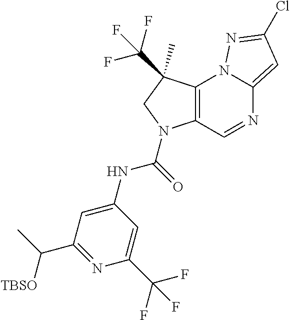 [SMILES: CC(O[Si](C)(C)C(C)(C)C)c1cc(NC(=O)N2C[C@@](C)(C(F)(F)F)c3c2cnc2cc(Cl)nn32)cc(C(F)(F)F)n1]

To a stirred solution of 2-(1-((tert-butyldimethylsilyl)oxy)ethyl)-6-(trifluoromethyl)pyridin-4-amine (139 mg, 433.8 μmol) in tetrahydrofuran (10 mL) were added triphosgene (64 mg, 216.9 μmol) and TEA (55 mg, 542.2 μmol). The resulting mixture was stirred at 40° C. for 2 h and then filtered. The filtrate was added to a solution of Method M1 isomer 2 (100 mg, 361.5 μmol) in tetrahydrofuran (5 mL). To this solution were added N,N-dimethylpyridin-4-amine (88 mg, 722.9 μmol) and TEA (366 mg, 3.6 mmol). The mixture was stirred at 40° C. for 2 h. The mixture was concentrated under vacuum. The residue was purified by Prep-HPLC and the collected fractions were lyophilized to afford (8R)—N-(2-(1-((tert-butyldimethylsilyl)oxy)ethyl)-6-(trifluoromethyl)pyridin-4-yl)-2-chloro-8-methyl-8-(trifluoromethyl)-7,8-dihydro-6H-pyrazolo[1,5-a]pyrrolo[2,3-e]pyrimidine-6-carboxamide (145 mg, 64% yield) as a white solid. LC-MS: m/z 623 [M+H]⁺.

### Step 6: (8R)-2-chloro-N-(2-(1-hydroxyethyl)-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

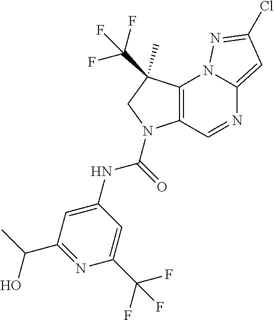 [SMILES: CC(O)c1cc(NC(=O)N2C[C@@](C)(C(F)(F)F)c3c2cnc2cc(Cl)nn32)cc(C(F)(F)F)n1]

To a mixture of (8R)—N-(2-(1-((tert-butyldimethylsilyl)oxy)ethyl)-6-(trifluoromethyl)pyridin-4-yl)-2-chloro-8-methyl-8-(trifluoromethyl)-7,8-dihydro-6H-pyrazolo[1,5-a]pyrrolo[2,3-e]pyrimidine-6-carboxamide (140 mg, 222.4 μmol) in tetrahydrofuran (2.0 mL) was added TFA (2.0 mL). The resulting mixture was stirred at 25° C. for 16 h. The resulting mixture was concentrated under vacuum. The residue was purified by Prep-HPLC and the collected fractions were lyophilized to afford (8R)-2-chloro-N-(2-(1-hydroxyethyl)-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide (80 mg, 71% yield) as a white solid. ¹H NMR (400 MHz, Methanol-d₄) δ: 9.37 (s, 1H), 8.04-7.99 (m, 2H), 6.78-6.77 (m, 1H), 4.90-4.82 (m, 2H), 4.21 (d, J=11.6 Hz, 1H), 2.03 (s, 3H), 1.48 (d, J=6.8 Hz, 3H). LC-MS: m/z 509 [M+H]⁺.

### Step 7: Separation of enantiomers to obtain (R)-2-chloro-N-(2-((S)-1-hydroxyethyl)-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(2-((R)-1-hydroxyethyl)-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

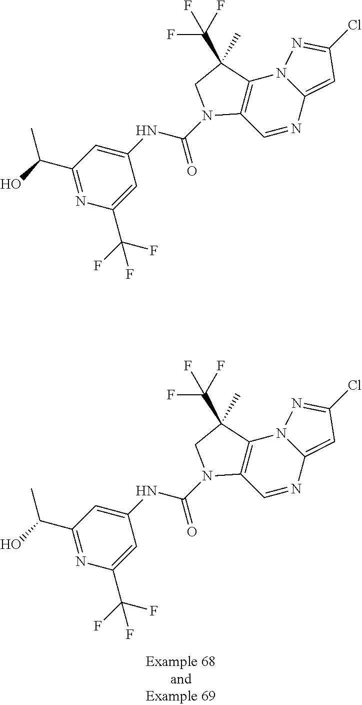 [SMILES: C[C@H](O)c1cc(NC(=O)N2C[C@@](C)(C(F)(F)F)c3c2cnc2cc(Cl)nn32)cc(C(F)(F)F)n1 ; C[C@@H](O)c1cc(NC(=O)N2C[C@@](C)(C(F)(F)F)c3c2cnc2cc(Cl)nn32)cc(C(F)(F)F)n1]

80 mg of (8R)-2-chloro-N-(2-(1-hydroxyethyl)-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide was submitted to chiral HPLC purification (Column: CHIRALCEL OD-H, 2*25 mm, 5 um; Mobile PhaseA:Hex(0.3% IPA)-HPLC, Mobile Phase B:IPA-HPLC; Flow rate: 20 mL/min; Gradient: 10 B to 10 B in 29 min; 220/254 nm; RT1:18.215; RT2:22.902; Injection Volume: 0.3 ml; Number Of Runs: 15). The first eluting isomer was concentrated and lyophilized to afford Example 68 (14.4 mg, 18% yield) as a white solid. The second eluting isomer was concentrated and lyophilized to afford Example 69 (13.5 mg, 17% yield) as a white solid. The enantiomers of Examples 68 and 69 can be prepared analogously using Method M1 isomer 1 in step 5.

Example 68: ¹H NMR (400 MHz, DMSO-d₆) δ 9.73 (s, 1H), 9.36 (s, 1H), 8.05 (d, J=1.6 Hz, 1H), 8.01 (d, J=1.6 Hz, 1H), 7.08 (s, 1H), 5.65 (d, J=4.0 Hz, 1H), 4.90 (d, J=11.6 Hz, 1H), 4.74-4.76 (m, 1H), 4.28 (d, J=11.6 Hz, 1H), 1.96 (s, 3H), 1.37 (d, J=6.4 Hz, 3H). LC-MS: m/z 509 [M+H]⁺.

Example 69: ¹H NMR (400 MHz, DMSO-d₆) δ 9.74 (s, 1H), 9.36 (s, 1H), 8.04 (d, J=2.0 Hz, 1H), 8.01 (d, J=2.0 Hz, 1H), 7.08 (s, 1H), 5.65 (d, J=4.2 Hz, 1H), 4.89 (d, J=11.6 Hz, 1H), 4.72-4.76 (m, 1H), 4.28 (d, J=11.6 Hz, 1H), 1.97 (s, 3H), 1.37 (d, J=6.8 Hz, 3H). LC-MS: m/z 509 [M+H]⁺.

### Method U2

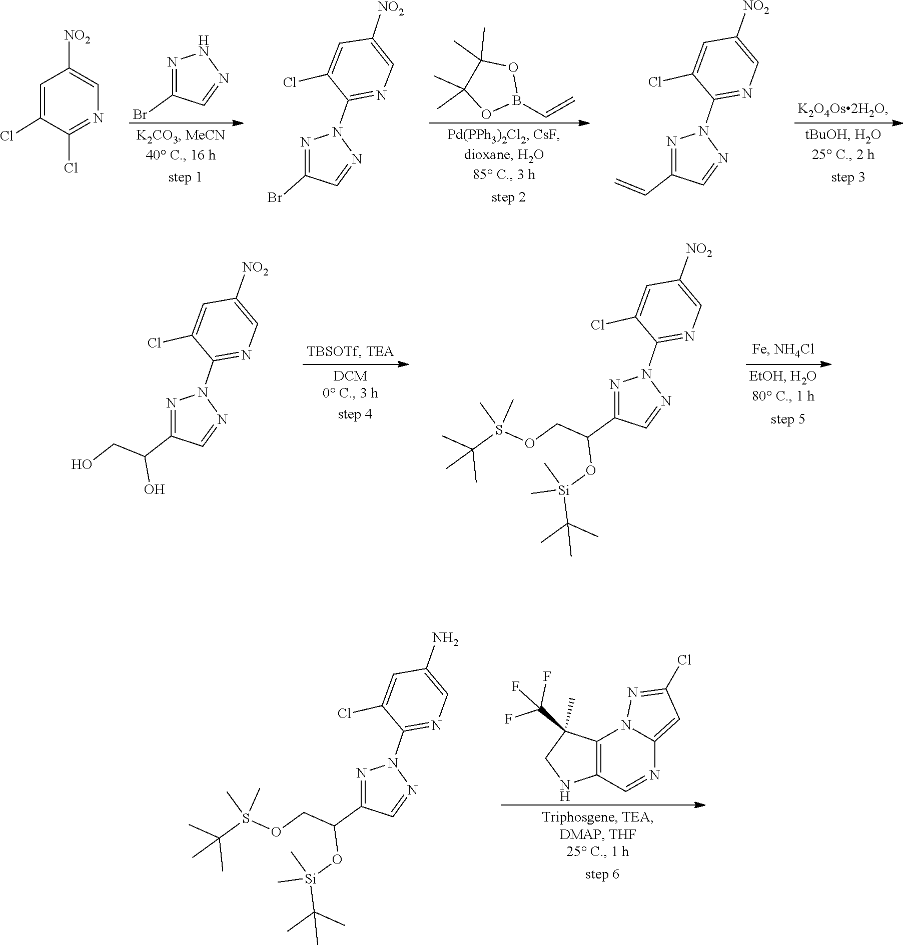

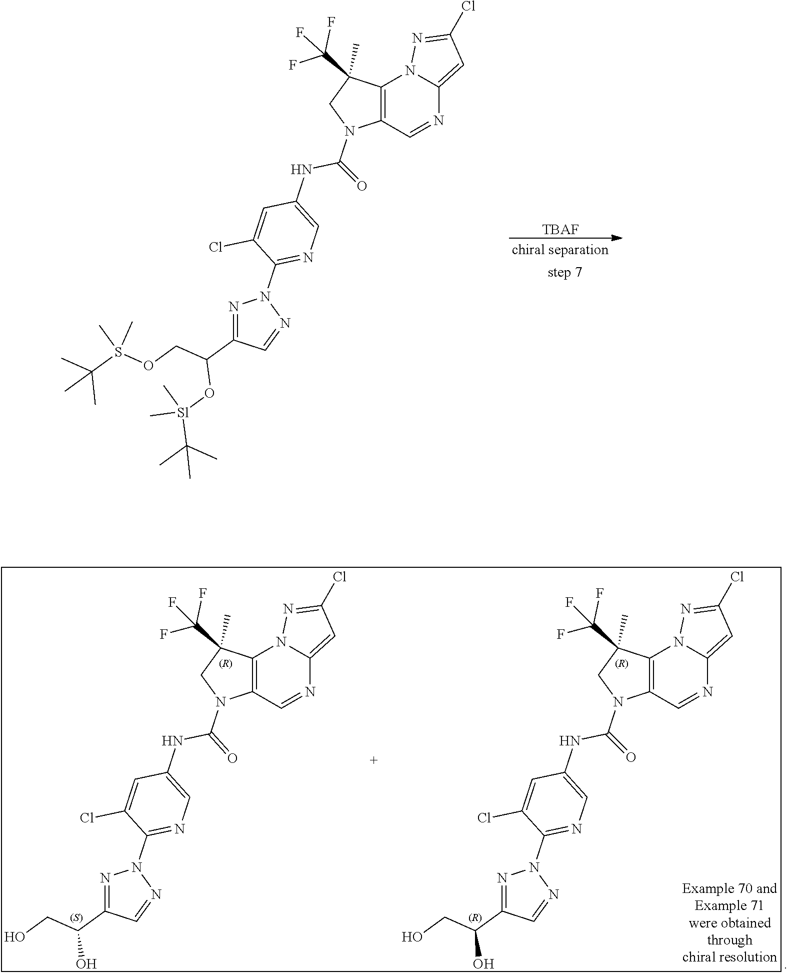

### Examples 70 and 71: Single enantiomers obtained from a racemic mixture containing (R)-2-chloro-N-(5-chloro-6-(4-((S)-1,2-dihydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyridine-6-carboxamide and (R)-2-chloro-N-(5-chloro-6-(4-((R)-1,2-dihydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyridine-6-carboxamide

### Step 1: 2-(4-bromo-2H-1,2,3-triazol-2-yl)-3-chloro-5-nitropyridine

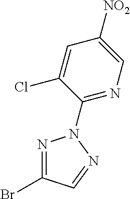 [SMILES: O=[N+]([O-])c1cnc(-n2ncc(Br)n2)c(Cl)c1]

To a stirred solution of 2,3-dichloro-5-nitropyridine (5 g, 25.9 mmol) in acetonitrile (100 mL) were added 4-bromo-2H-1,2,3-triazole (4.2 g, 28.5 mmol) and K₂CO₃ (7.2 g, 51.8 mmol). The resulting mixture was stirred at 40° C. for 16 h. The reaction mixture was filtered and washed with ethyl acetate (3×50 mL). The combined organic layers were dried over anhydrous sodium sulfate and concentrated under vacuum. The residue was purified by column chromatography on silica gel using 83% petroleum ether and 17% ethyl acetate as eluent to afford 2-(4-bromo-2H-1,2,3-triazol-2-yl)-3-chloro-5-nitropyridine (3.6 g, 62% yield) as a white solid. ¹H NMR (300 MHz, DMSO-d) S 9.39 (d, J=3 Hz, 1H), 9.16 (d, J=3 Hz, 1H), 8.55 (s, 1H); LC-MS: m/z 304 [M+H]⁺

### Step 2: 3-chloro-5-nitro-2-(4-vinyl-2H-1,2,3-triazol-2-yl)pyridine

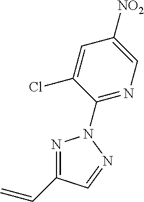 [SMILES: C=Cc1cnn(-c2ncc([N+](=O)[O-])cc2Cl)n1]

To a solution of 2-(4-bromo-2H-1,2,3-triazol-2-yl)-3-chloro-5-nitropyridine (3.0 g, 9.9 mmol) in dioxane (60 mL) and water (6 mL) were added CsF (4.5 g, 29.7 mmol), 4,4,5,5-tetramethyl-2-vinyl-1,3,2-dioxaborolane (2.3 g, 14.9 mmol), Pd(PPh₃)₂Cl₂ (0.7 g, 1.0 mmol) under nitrogen atmosphere. The resulting mixture was stirred at 85° C. for 3 h. The reaction mixture was quenched with water (200 mL). The resulting solution was extracted with ethyl acetate (3×200 mL). The combined organic layers were dried over anhydrous sodium sulfate and concentrated under vacuum. The residue was purified by column chromatography on silica gel using 83% petroleum ether and 17% ethyl acetate as eluent to afford 3-chloro-5-nitro-2-(4-vinyl-2H-1,2,3-triazol-2-yl)pyridine (690 mg, 28% yield) as a yellow solid. H NMR (400 MHz, DMSO-d₆) δ 9.39 (d, J=4 Hz, 1H), 9.11 (d, J=4 Hz, 1H), 8.56 (s, 1H), 6.83-7.39 (m, 1H), 6.13-6.18 (m, 1H), 5.63-5.74 (m, 1H); LC-MS: m/z 252 [M+H]⁺.

### Step 3: 1-(2-(3-chloro-5-nitropyridin-2-yl)-2H-1,2,3-triazol-4-yl)ethane-1,2-diol

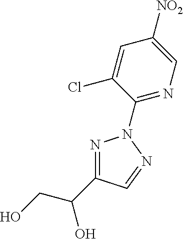 [SMILES: O=[N+]([O-])c1cnc(-n2ncc(C(O)CO)n2)c(Cl)c1]

A solution of 3-chloro-5-nitro-2-(4-vinyl-2H-1,2,3-triazol-2-yl)pyridine (690 mg, 2.7 mmol) in tert-Butanol (12 mL) and water (3 mL) were added K₂O₄Os·2H₂O (354 mg, 1 mmol) and 4-methylmorpholine 4-oxide (632 mg, 5.4 mmol). The reaction solution was stirred at 25° C. for 2 h. The reaction mixture was quenched with water (50 mL). The resulting solution was extracted with ethyl acetate (3×50 mL). The combined organic layers were dried over anhydrous sodium sulfate and concentrated under vacuum. The residue was purified by column chromatography on silica gel using 80% methanol and 20% dichloromethane as eluent to afford 1-(2-(3-chloro-5-nitropyridin-2-yl)-2H-1,2,3-triazol-4-yl)ethane-1,2-diol (500 mg, 61%) as an off-white solid. ¹H NMR (300 MHz, DMSO-d₆) δ 9.38 (d, J=3 Hz, 1H), 9.11 (d, J=3 Hz, 1H), 8.2 (s, 1H), 5.71-5.73 (m, 1H), 4.80-4.95 (m, 2H), 3.61-3.69 (m, 2H); LC-MS: m/z 286 [M+H]⁺.

### Step 4: 3-chloro-5-nitro-2-(4-(2,2,3,3,8,8,9,9-octamethyl-4,7-dioxa-3,8-disiladecan-5-yl)-2H-1,2,3-triazol-2-yl)pyridine

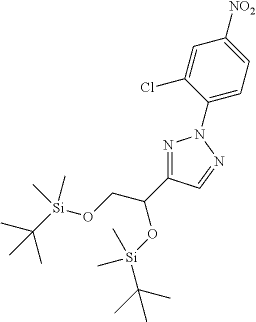 [SMILES: CC(C)(C)[Si](C)(C)OCC(O[Si](C)(C)C(C)(C)C)c1cnn(-c2ccc([N+](=O)[O-])cc2Cl)n1]

To a solution of 1-(2-(3-chloro-5-nitropyridin-2-yl)-2H-1,2,3-triazol-4-yl)ethane-1,2-diol (500 mg, 1.8 mmol) in dichloromethane (34 mL) were added tert-butyldimethylsilyl trifluoromethanesulfonate (2.1 g, 8.1 mmol) and DIEA (1.6 g, 12.3 mmol) at 0° C. The reaction mixture was stirred at 0° C. for 3 h. The mixture was concentrated under vacuum. The residue was diluted with water (50 mL) and then extracted with ethyl acetate (3×50 mL). The combined organic layers were washed with saturated aqueous NaHCO₃ (50 mL). The resulted solution was dried over anhydrous sodium sulfate and concentrated under vacuum. The residue was purified by column chromatography on silica gel using 83% petroleum ether and 17% ethyl acetate as eluent to afford 3-chloro-5-nitro-2-(4-(2,2,3,3,8,8,9,9-octamethyl-4,7-dioxa-3,8-disiladecan-5-yl)-2H-1,2,3-triazol-2-yl)pyridine (700 mg, 79% yield) as a yellow oil. LC-MS: m/z 513 [M+H]⁺.

### Step 5: 5-chloro-6-(4-(2,2,3,3,8,8,9,9-octamethyl-4,7-dioxa-3,8-disiladecan-5-yl)-2H-1,2,3-triazol-2-yl)pyridin-3-amine

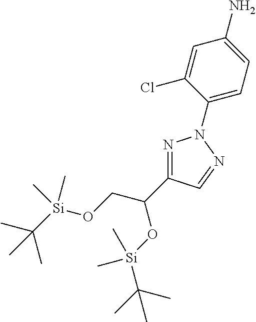 [SMILES: CC(C)(C)[Si](C)(C)OCC(O[Si](C)(C)C(C)(C)C)c1cnn(-c2ccc(N)cc2Cl)n1]

To a solution of 3-chloro-5-nitro-2-(2,2,3,3,8,8,9,9-octamethyl-4,7-dioxa-3,8-disiladecan-5-yl)pyridine (200 mg, 390.0 umol) in ethanol (9 mL) and water (3 mL) were added Fe (108 mg, 2.0 mmol) and NH₄Cl (83 mg, 1.6 mmol). The resulting mixture was stirred at 80° C. for 1 h. The reaction mixture was quenched by the addition of water (50 mL). The resulting solution was extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate as eluent to afford 5-chloro-6-(4-(2,2,3,3,8,8,9,9-octamethyl-4,7-dioxa-3,8-disiladecan-5-yl)-2H-1,2,3-triazol-2-yl)pyridin-3-amine (120 mg, 64% yield) as a white solid. ¹H NMR (300 MHz, DMSO-d₆) δ 7.91 (s, 1H), 7.82 (d, J=3 Hz, 1H), 7.19 (d, J=3 Hz, 1H), 6.17 (s, 1H), 4.95-4.98 (m, 1H), 3.77-3.79 (m, 2H), 0.85 (s, 18H), 0.08 (s, 12H); LC-MS: m/z 483[M+H]⁺.

### Step 6: (8R)-2-chloro-N-(5-chloro-6-(4-(2,2,3,3,8,8,9,9-octamethyl-4,7-dioxa-3,8-disiladecan-5-yl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyridine-6-carboxamide

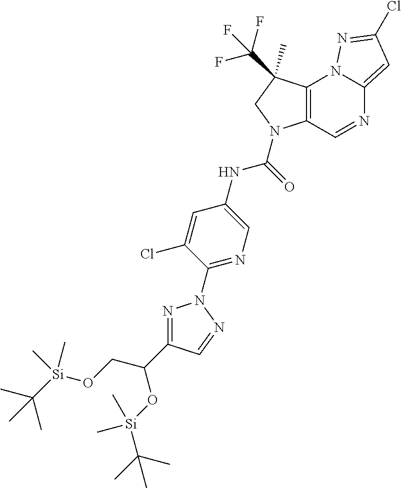 [SMILES: CC(C)(C)[Si](C)(C)OCC(O[Si](C)(C)C(C)(C)C)c1cnn(-c2ncc(NC(=O)N3C[C@@](C)(C(F)(F)F)c4c3cnc3cc(Cl)nn43)cc2Cl)n1]

To a mixture of 5-chloro-6-(4-(2,2,3,3,8,8,9,9-octamethyl-4,7-dioxa-3,8-disiladecan-5-yl)-2H-1,2,3-triazol-2-yl)pyridin-3-amine (53 mg, 109.7 μmol) in tetrahydrofuran (4 mL) were added triphosgene (13 mg, 43.5 μmol) and TEA (11 mg, 180.7 μmol) at 25° C. The resulting mixture was stirred at 25° C. for 1 h. and then filtered. The filtrate was added to a solution of Method M1 isomer 2 (20 mg, 72.5 μmol) in tetrahydrofuran (1 mL). To this solution was then added TEA (121 mg, 725 μmol) and N, N-Dimethylpyridin-4-amine (18 mg, 144.9 μmol). The reaction mixture was stirred for 1 h at 40° C. The residue was diluted with water (50 mL) and then extracted with ethyl acetate (3×50 mL). The combined organic layers were washed with brine (50 mL), dried over anhydrous sodium sulfate and concentrated under vacuum. The residue was purified by column chromatography on silica gel using 50% petroleum ether and 50% ethyl acetate as eluent to afford (8R)-2-chloro-N-(5-chloro-6-(4-(2,2,3,3,8,8,9,9-octamethyl-4,7-dioxa-3,8-disiladecan-5-yl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyridine-6-carboxamides (40 mg, 70% yield) an off-white solid. LC-MS: m/z 786 [M+H]⁺.

### Step 7: (8R)-2-chloro-N-(5-chloro-6-(4-(1,2-dihydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyridine-6-carboxamide

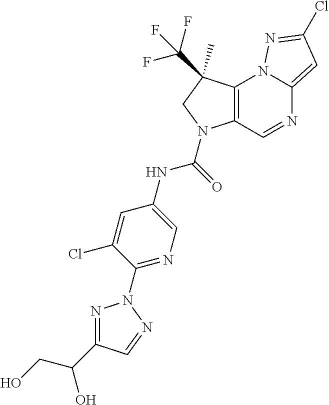 [SMILES: C[C@@]1(C(F)(F)F)CN(C(=O)Nc2cnc(-n3ncc(C(O)CO)n3)c(Cl)c2)c2cnc3cc(Cl)nn3c21]

To a solution of (8R)-2-chloro-N-(5-chloro-6-(4-(2,2,3,3,8,8,9,9-octamethyl-4,7-dioxa-3,8-disiladecan-5-yl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyridine-6-carboxamides (40 mg, 51.0 μmol) in tetrahydrofuran (5 mL) was added TBAF (0.5 mL, 510 μmol, 1 M in tetrahydrofuran) at 25° C. The resulting mixture was stirred at 25° C. for 4 h. The reaction mixture was concentrated under reduced pressure. The residue was diluted with water (50 mL). The resulting solution was extracted with ethyl acetate (3×50 mL). The combined organic layers were washed with saturated aqueous NH₄Cl (3×50 mL). The resulting solution was dried over anhydrous sodium sulfate and concentrated under vacuum. The residue was purified by Prep-TLC with ethyl acetate to afford 30 mg of crude product (90% purity). The crude product was submitted to Prep-HPLC purification and the collected fractions were lyophilized to afford (8R)-2-chloro-N-(5-chloro-6-(4-(1,2-dihydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyridine-6-carboxamide (20 mg, 71% yield) as a white solid. LC-MS: m/z 558 [M+H]⁺

### Step 8: Separation of enantiomers to obtain (R)-2-chloro-N-(5-chloro-6-(4-((S)-1,2-dihydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyridine-6-carboxamide and (R)-2-chloro-N-(5-chloro-6-(4-((R)-1,2-dihydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyridine-6-carboxamide

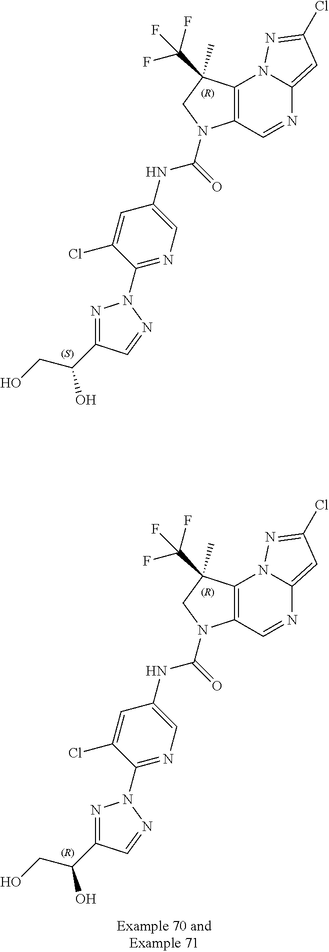

20 mg of (8R)-2-chloro-N-(5-chloro-6-(4-(1,2-dihydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyridine-6-carboxamide was submitted to chiral HPLC purification (Column: CHIRAL ART Cellulose-SB, 2*25 cm, 5 um; Mobile Phase A:Hex(0.5% 2M NH₃-MeOH)—HPLC, Mobile Phase B:EtOH—HPLC; Flow rate: 20 mL/L/min; Gradient: 30 B to 30 B in 30 min; 220/254 nm; RT1:20.983; RT2: 25.151; Injection Volume: 0.7 ml; Number Of Runs: 4). The first eluting isomer was concentrated and lyophilized to afford Example 70 (4.3 mg, 10% yield) as a white solid. The second eluting isomer was concentrated and lyophilized to afford Example 71 (5 mg, 10% yield) as a white solid. The enantiomers of Examples 70 and 71 can be prepared analogously using Method M1 isomer 1 in step 6.

Example 70: ¹H NMR (400 MHz, DMSO-d₆) δ 9.66 (s, 1H), 9.36 (s, 1H), 8.73 (d, J=2.4 Hz, 1H), 8.49 (d, J=2.4 Hz, 1H), 8.02 (s, 1H), 7.07 (s, 1H), 5.59 (d, J=5.2 Hz, 1H), 4.83-4.89 (m, 2H), 4.77-4.78 (m, 1H), 4.30 (d, J=12 Hz, 1H), 3.61-3.67 (m, 2H), 1.98 (s, 3H); LC-MS: m/z 558 [M+H]⁺.

Example 71: ¹H NMR (400 MHz, DMSO-d₆) δ 9.66 (s, 1H), 9.36 (s, 1H), 8.73 (d, J=2.4 Hz, 1H), 8.49 (d, J=2.4 Hz, 1H), 8.02 (s, 1H), 7.07 (s, 1H), 5.59 (d, J=5.2 Hz, 1H), 4.83-4.89 (m, 2H), 4.77-4.78 (m, 1H), 4.30 (d, J=12 Hz, 1H), 3.63-3.67 (m, 2H), 1.98 (s, 3H); LC-MS: m/z 558 [M+H]⁺.

### Method V2

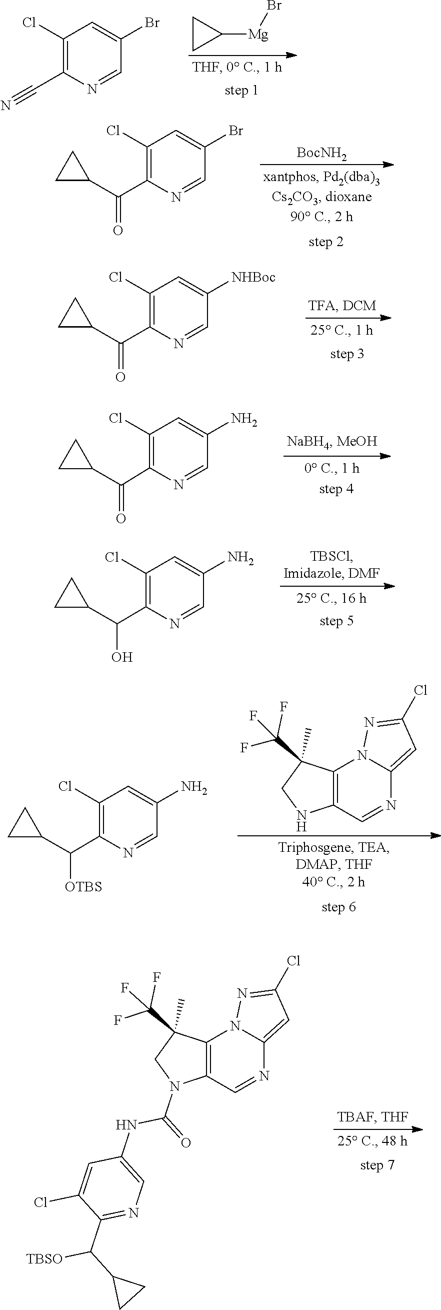

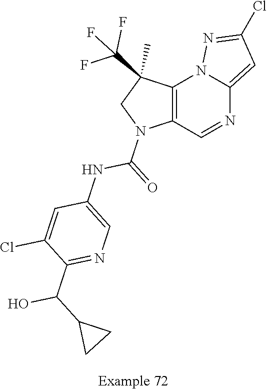 [SMILES: C[C@@]1(C(F)(F)F)CN(C(=O)Nc2cnc(C(O)C3CC3)c(Cl)c2)c2cnc3cc(Cl)nn3c21]

### Examples 72: (8R)-2-chloro-N-(5-chloro-6-(cyclopropyl(hydroxy)methyl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: (5-bromo-3-chloropyridin-2-yl)(cyclopropyl)methanone

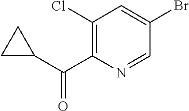 [SMILES: O=C(c1ncc(Br)cc1Cl)C1CC1]

To a stirred solution of 5-bromo-3-chloropicolinonitrile (2.0 g, 9.2 mmol) in tetrahydrofuran (20 mL) was added cyclopropylmagnesium bromide (2.5 g, 18.4 mmol) dropwise at 0° C. The mixture was stirred for 1 h at 0° C. The reaction mixture was quenched by the addition of saturated aqueous NH₄Cl solution (40 mL) at 0° C. The resulting solution was extracted with ethyl acetate (3×50 mL). The combined organic layers were dried over anhydrous sodium sulfate and concentrated under vacuum. The residue was purified by column chromatography on silica gel using 80% petroleum ether and 20% ethyl acetate as eluent to afford (5-bromo-3-chloropyridin-2-yl)(cyclopropyl)methanone (1.7 g, 68% yield) as a yellow solid. ¹H NMR (400 MHz, Chloroform-d) δ8.63 (d, J=2.0 Hz, 1H), 7.98 (d, J=2.0 Hz, 1H), 2.88-2.97 (m, 1H), 1.22-1.34 (m, 2H), 1.06-1.19 (in, 2H). LC-MS: m/z 260, 262 [M+H]⁺.

### Step 2: tert-butyl (5-chloro-6-(cyclopropanecarbonyl)pyridin-3-yl)carbamate

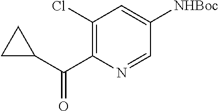 [SMILES: CC(C)(C)OC(=O)Nc1cnc(C(=O)C2CC2)c(Cl)c1]

To a stirred solution of (5-bromo-3-chloropyridin-2-yl)(cyclopropyl)methanone (1.4 g, 5.3 mmol) and tert-butyl carbamate (944 mg, 8.0 mmol) in dioxane (30 mL) were added Xantphos (932 mg, 1.6 mmol), Pd₂(dba)₃ (618 mg, 1.1 mmol) and Cs₂CO₃ (5.2 g, 16.1 mmol) under nitrogen atmosphere. The resulting mixture was stirred at 90° C. for 2 h. The solid was filtered out. The filtrate was concentrated under vacuum. The residue was purified by column chromatography on silica gel using 50% petroleum ether and 50% ethyl acetate as eluent to afford tert-butyl (5-chloro-6-(cyclopropanecarbonyl)pyridin-3-yl)carbamate (1 g, 62% yield) as a yellow solid. ¹H NMR (400 MHz, Chloroform-d) δ 8.40 (d, J=2.4 Hz, 1H), 8.21 (d, J=2.4 Hz, 1H), 3.00-3.10 (m, 1H), 1.52 (s, 9H), 1.22-1.27 (m, 2H), 1.02-1.09 (m, 2H). LC-MS: m/z 297 [M+H]⁺.

### Step 3: (5-amino-3-chloropyridin-2-yl)cyclopropyl)methanone

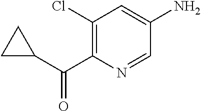 [SMILES: Nc1cnc(C(=O)C2CC2)c(Cl)c1]

To a stirred solution of tert-butyl (5-chloro-6-(cyclopropanecarbonyl)pyridin-3-yl)carbamate (400 mg, 1.3 mmol) in dichloromethane (20 mL) was added TFA (4 mL). The mixture was stirred at 25° C. for 1 h. The resulting mixture was concentrated under vacuum. The residue was added saturated aqueous NaHCO₃ (40 mL). The resulting solution was extracted with ethyl acetate (3×40 mL). The combined organic layers were washed with brine (50 mL), dried over anhydrous sodium sulfate and concentrated under vacuum. The residue was purified by column chromatography on silica gel using 50% petroleum ether and 50% ethyl acetate as eluent to afford (5-amino-3-chloropyridin-2-yl)(cyclopropyl)methanone (170 mg, 64% yield) as a yellow solid. ¹H NMR (300 MHz, Chloroform-d) δ 8.02 (d, J=2.4 Hz, 1H), 7.00 (d, J=2.4 Hz, 1H), 3.53-4.26 (m, 2H), 3.04-3.26 (m, 1H), 1.16-1.26 (m, 2H), 0.96-1.06 (m, 2H). LC-MS: m/z 197 [M+H]⁺.

### Step 4: (5-amino-3-chloropyridin-2-yl)(cyclopropyl)methanol

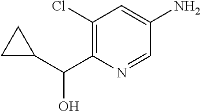 [SMILES: Nc1cnc(C(O)C2CC2)c(Cl)c1]

To a stirred solution of (5-amino-3-chloropyridin-2-yl)(cyclopropyl)methanone (210 mg, 1.1 mmol) in methanol (4 mL) was added sodium tetrahydroborate (48 mg, 1.3 mmol) at 0° C. under nitrogen atmosphere. The mixture was stirred at 0° C. for 1 h. The reaction mixture was quenched with water/ice (10 mL). The resulting mixture was concentrated under reduced pressure. The resulting solution was extracted with ethyl acetate (3×10 mL). The combined organic layers were dried over anhydrous sodium sulfate and concentrated under vacuum. The residue was purified by column chromatography on silica gel using 95% dichloromethane and 5% methanol as eluent to afford (5-amino-3-chloropyridin-2-yl)(cyclopropyl)methanol (140 mg, 65% yield) as a white solid. ¹H NMR (400 MHz, Methanol-d₄) δ 7.91 (d, J=2.4 Hz, 1H), 7.05 (d, J=2.4 Hz, 1H), 4.38 (d, J=8.0 Hz, 1H), 1.30-1.44 (m, 1H), 0.94-1.09 (m, 1H), 0.27-0.64 (m, 3H). LC-MS: m/z 199 [M+H]⁺.

### Step 5: 6-(((tert-butyldimethylsilyl)oxy)(cyclopropyl)methyl)-5-chloropyridin-3-amine

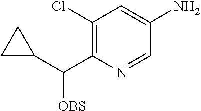

To a solution of (5-amino-3-chloropyridin-2-yl)(cyclopropyl)methanol (130 mg, 654.4 μmol) in N,N-dimethylformamide (5 mL) were added tert-Butyldimethylsilyl trifluoromethanesulfonate (542 mg, 3.6 mmol) and imidazole (300 mg, 4.4 mmol) at 0° C. The reaction mixture was stirred at 25° C. for 16 h. The mixture was concentrated under vacuum. The residue was diluted with water (50 mL). The resulting solution was then extracted with ethyl acetate (3×50 mL). The combined organic layers were washed with saturated aqueous NaHCO₃ (50 mL), dried over anhydrous sodium sulfate and concentrated under vacuum. The residue was purified by column chromatography on silica gel using 50% petroleum ether and 50% ethyl acetate as eluent to afford 6-(((tert-butyldimethylsilyl)oxy)(cyclopropyl)methyl)-5-chloropyridin-3-amine (40 mg, 19% yield) as a colorless oil. LC-MS: m/z 313 [M+H]⁺

### Step 6: (8R)—N-(6-(((tert-butyldimethylsilyl)oxy)(cyclopropyl)methyl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide

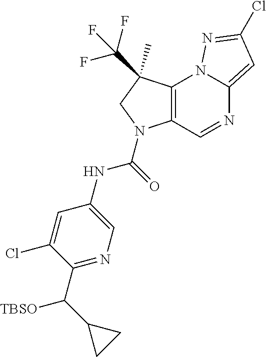 [SMILES: CC(C)(C)[Si](C)(C)OC(c1ncc(NC(=O)N2C[C@@](C)(C(F)(F)F)c3c2cnc2cc(Cl)nn32)cc1Cl)C1CC1]

To a stirred solution of Method M1 isomer 2 (28 mg, 101.2 μmol) in tetrahydrofuran (4 mL) were added triphosgene (18 mg, 60.7 μmol) and TEA (11 mg, 107.7 μmol) at 0° C. The resulting mixture was stirred at 25° C. for 0.5 h and then filtered. The filtrate was added to a solution of 6-(((tert-butyldimethylsilyl)oxy)(cyclopropyl)methyl)-5-chloropyridin-3-amine (38 mg, 121.1 μmol) in tetrahydrofuran (2 mL). To this solution was then added N,N-dimethylpyridin-4-amine (24 mg, 202.3 μmol) and TEA (102 mg, 1.0 mmol). The mixture was stirred at 40° C. for 2 h. The mixture was poured into water (10 mL) and extracted with ethyl acetate (3×10 mL). The combined organic layers were washed with brine (20 mL), dried over anhydrous sodium sulfate and concentrated under vacuum. The residue was purified by Prep-TLC using 95% dichloromethane and 5% methanol as eluent to afford (8R)—N-(6-(((tert-butyldimethylsilyl)oxy)(cyclopropyl)methyl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide (20 mg, 32% yield) as a white solid. LC-MS: m/z 615 [M+H]⁺.

### Step 7: (8R)-2-chloro-N-(5-chloro-6-(cyclopropyl(hydroxy)methyl)pyridin-3-yl)-8-methyl-8-(trifluoromethyl)-7,8-dihydro-6H-pyrazolo[1,5-a]pyrrolo[2,3-e]pyrimidine-6-carboxamide

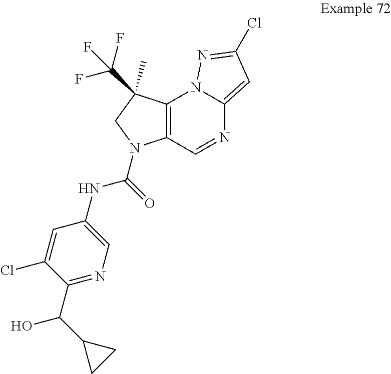 [SMILES: C[C@@]1(C(F)(F)F)CN(C(=O)Nc2cnc(C(O)C3CC3)c(Cl)c2)c2cnc3cc(Cl)nn3c21]

To a stirred solution of (8R)—N-(6-(((tert-butyldimethylsilyl)oxy)(cyclopropyl)methyl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide (15 mg, 24.3 μmol) in tetrahydrofuran (4 mL) was added TBAF (244 μL, 244 μmol, 1 M in tetrahydrofuran) at 25° C. The resulting mixture was stirred at 25° C. for 48 h. The resulting mixture was concentrated under reduced pressure. The residue was purified by Prep-TLC using ethyl acetate (100%) as eluent to afford 10 mg of crude product. The crude product was purified by Prep-HPLC and the collected fractions were lyophilized to give (8R)-2-chloro-N-(5-chloro-6-(cyclopropyl(hydroxy)methyl)pyridin-3-yl)-8-methyl-8-(trifluoromethyl)-7,8-dihydro-6H-pyrazolo[1,5-a]pyrrolo[2,3-e]pyrimidine-6-carboxamide (2.8 mg, 22% yield) as white solid. The enantiomer of Example 72 can be prepared analogously using Method M1 isomer 1.

Example 72: ¹H NMR (400 MHz, DMSO-d₆) δ 9.38 (s, 1H), 9.33 (s, 1H), 8.69 (t, J=2.0 Hz, 1H), 8.14 (t, J=2.0 Hz, 1H), 7.06 (s, 1H), 5.16 (d, J=6.4 Hz, 1H), 4.77-4.85 (m, 1H), 4.20-4.35 (m, 2H), 1.98 (s, 3H), 1.31-1.44 (m, 1H), 0.19-0.53 (m, 4H). LC-MS: m/z 501 [M+H]⁺

### Method W2

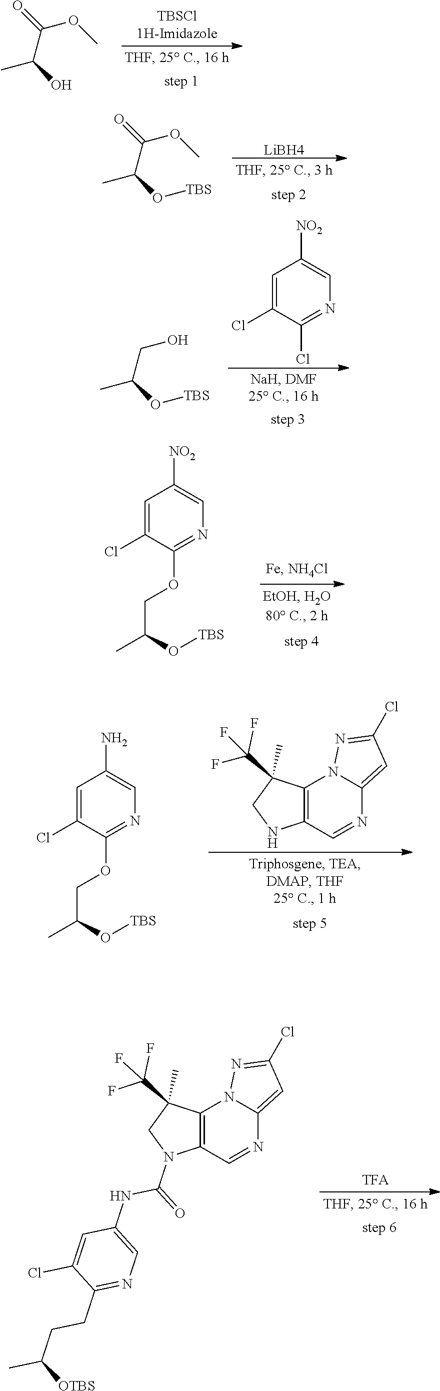

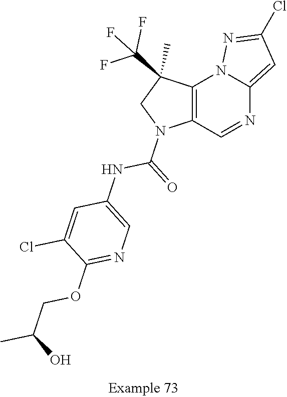 [SMILES: C[C@H](O)COc1ncc(NC(=O)N2C[C@@](C)(C(F)(F)F)c3c2cnc2cc(Cl)nn32)cc1Cl]

### Example 73: (R)-2-chloro-N-(5-chloro-6-((S)-2-hydroxypropoxy)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: methyl (S)-2-((tert-butyldimethylsilyl)oxy)propanoate

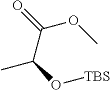

To a stirred solution of methyl (S)-2-hydroxypropanoate (2.0 g, 19.2 mmol) and imidazole (3.9 g, 57.6 mmol) in tetrahydrofuran (50 mL) were added tert-butylchlorodimethylsilane (3.5 g, 23.0 mmol) at 0° C. The reaction mixture was stirred at 25° C. for 16 h. The reaction mixture was quenched with water (50 mL). The resulting solution was extracted with ethyl acetate (2×100 mL). The combined organic layers were dried over anhydrous sodium sulfate and concentrated under vacuum to give methyl (S)-2-((tert-butyldimethylsilyl)oxy)propanoate (4.0 g, 95% yield) as a colorless oil which was used to the next step without purification. ¹H NMR (400 MHz, Chloroform-d) δ: 4.34 (q, J=6.8 Hz, 1H), 3.72 (s, 3H), 1.39 (d, J=6.8 Hz, 3H), 0.91 (s, 9H), 0.11 (s, 3H), 0.09 (s, 3H).

### Step 2: (S)-2-((tert-butyldimethylsilyl)oxy)propan-1-ol

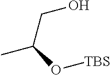

To a stirred solution of methyl (S)-2-((tert-butyldimethylsilyl)oxy)propanoate (2.0 g, 9.2 mmol) in tetrahydrofuran (20 mL) was added lithium tetrahydroborate (399 mg, 18.3 mmol) at 0° C. The resulting mixture was stirred at 25° C. for 3 h. The reaction mixture was quenched with water (10 mL). The resulting solution was extracted with dichloromethane (2×10 mL). The combined organic layers were dried over anhydrous sodium sulfate and concentrated under vacuum. The residue was purified by column chromatography on silica gel using 80% petroleum ether and 20% ethyl acetate as eluent to afford (S)-2-((tert-butyldimethylsilyl)oxy)propan-1-ol (1.3 g, 44% yield) as a yellow oil. ¹H NMR (400 MHz, DMSO-d₆) δ: 4.61 (t, J=5.6 Hz, 1H), 3.70-3.75 (m, 1H), 3.28-3.35 (m, 1H), 3.14-3.23 (m, 1H), 1.09 (d, J=6.4 Hz, 3H), 0.90 (s, 9H), 0.05 (s, 6H).

### Step 3: (S)-2-(2-((tert-butyldimethylsilyl)oxy)propoxy)-3-chloro-5-nitropyridine

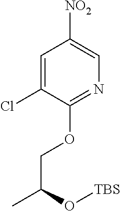

To a stirred solution of (S)-2-((tert-butyldimethylsilyl)oxy)propan-1-ol (1.5 g, 7.9 mmol) in N,N-dimethylformamide (20 mL) was added NaH (473 mg, 11.8 mmol, 60% in mineral oil) at 0° C. The reaction mixture was stirred at 0° C. for 1 h. Then 2,3-dichloro-5-nitropyridine (1.5 g, 7.9 mmol) was added and the reaction mixture was stirred for 16 h at 25° C. The reaction mixture was quenched with water (20 mL). The resulting solution was extracted with ethyl acetate (2×100 mL). The combined organic layers were dried over anhydrous sodium sulfate and concentrated under vacuum. The residue was purified by column chromatography on silica gel using 95% petroleum ether and 5% ethyl acetate as eluent to afford (S)-2-(2-((tert-butyldimethylsilyl)oxy)propoxy)-3-chloro-5-nitropyridine (600 mg, 16% yield) as a yellow oil. ¹H NMR (400 MHz, Chloroform-d) δ: 8.93-8.96 (m, 1H), 8.47-8.44 (m, 1H), 4.44 (dd, J=10.8, 7.2 Hz, 1H), 4.31 (dd, J=10.8, 4.4 Hz, 1H), 4.21-4.25 (m, 1H), 1.26 (d, J=6.4 Hz, 3H), 0.87 (s, 9H), 0.06 (s, 3H), 0.01 (s, 3H). LC-MS: m/z 347 [M+H]⁺.

### Step 4: (S)-6-(2-((tert-butyldimethylsilyl)oxy)propoxy)-5-chloropyridin-3-amine

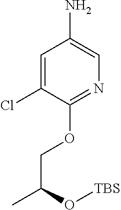

To a stirred mixture of (S)-2-(2-((tert-butyldimethylsilyl)oxy)propoxy)-3-chloro-5-nitropyridine (580 mg, 1.6 mmol) and NH₄Cl (179 mg, 3.3 mmol) in ethanol (3 mL) and water (3 mL) was added Fe (467 mg, 8.4 mmol). The reaction mixture was stirred at 80° C. for 2 h. The resulting mixture was filtered, and the filtrate was extracted with ethyl acetate (2×50 mL). The combined organic layers were dried over anhydrous sodium sulfate and concentrated under vacuum. The residue was purified by Prep-HPLC purification and the collected fractions were concentrated to afford (S)-6-(2-((tert-butyldimethylsilyl)oxy)propoxy)-5-chloropyridin-3-amine (140 mg, 19% yield) as a yellow oil. ¹H NMR (400 MHz, DMSO-d₄) S 7.41 (d, J=2.6 Hz, 1H), 7.13 (d, J=2.8 Hz, 1H), 5.00 (s, 2H), 3.96-4.16 (m, 3H), 1.14 (d, J=6.4 Hz, 3H), 0.84 (s, 9H), 0.05 (s, 3H), 0.02 (s, 3H). LC-MS: m/z 317 [M+H]⁺.

### Step 5: (R)—N-(6-((S)-2-((tert-butyldimethylsilyl)oxy)propoxy)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide

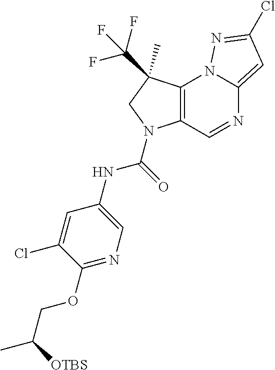 [SMILES: C[C@@H](COc1ncc(NC(=O)N2C[C@@](C)(C(F)(F)F)c3c2cnc2cc(Cl)nn32)cc1Cl)O[Si](C)(C)C(C)(C)C]

To a stirred solution of (S)-6-(2-((tert-butyldimethylsilyl)oxy)propoxy)-5-chloropyridin-3-amine (110 mg, 347 μmol) in tetrahydrofuran (5 mL) were added triphosgene (41 mg, 139 μmol) and TEA (35 mg, 347 μmol). The resulting mixture was stirred at 25° C. for 1 h and then filtered. The filtrate was added to a solution of Method M1 isomer 2 (64 mg, 231 μmol) in tetrahydrofuran (5 mL). To this solution were added N,N-dimethylpyridin-4-amine (57 mg, 463 μmol) and TEA (234 mg, 2.3 mmol). The mixture was stirred at 40° C. for 2 h. The reaction mixture was quenched by the addition of methanol (2 mL) and the mixture was concentrated under vacuum. The residue was diluted with water (10 mL). The resulting mixture was extracted with ethyl acetate (2×20 mL). The combined organic layers were dried over anhydrous sodium sulfate and concentrated under vacuum. The residue was purified by column chromatography on silica gel using 50% petroleum ether and 50% ethyl acetate as eluent to afford (R)—N-(6-((S)-2-((tert-butyldimethylsilyl)oxy)propoxy)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide (80 mg, 45% yield) as a yellow solid. ¹H NMR (400 MHz, Chloroform-d) δ: 9.39 (s, 1H), 7.95-8.04 (m, 2H), 6.75 (s, 1H), 6.33 (s, 1H), 4.54 (d, J=10.0 Hz, 1H), 4.11-4.36 (m, 3H), 4.02 (d, J=10.4 Hz, 1H), 2.07 (s, 3H), 1.25 (d, J=6.0 Hz, 3H), 0.89 (s, 9H), 0.10 (s, 3H), 0.08 (s, 3H). LC-MS: m/z 619 [M+H]⁺.

### Step 6: (R)-2-chloro-N-(5-chloro-6-((S)-2-hydroxypropoxy)pyridin-3-yl)-8-methyl-8-(trifluoromethyl)-7,8-dihydro-6H-pyrazolo[1,5-a]pyrrolo[2,3-e]pyrimidine-6-carboxamide

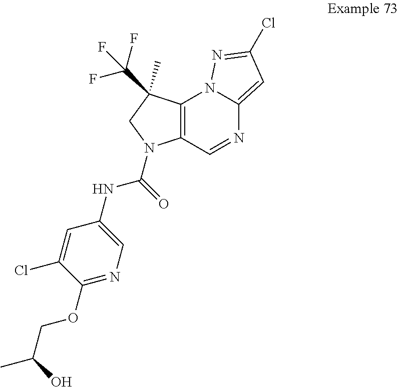 [SMILES: C[C@H](O)COc1ncc(NC(=O)N2C[C@@](C)(C(F)(F)F)c3c2cnc2cc(Cl)nn32)cc1Cl]

To a stirred solution of (R)—N-(6-((S)-2-((tert-butyldimethylsilyl)oxy)propoxy)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide (70 mg, 113 μmol) in tetrahydrofuran (2 mL) was added TFA (0.5 mL). The reaction mixture was stirred at 25° C. for 16 h. The mixture was concentrated under vacuum. The residue was purified by Prep-HPLC purification and the collected fractions were lyophilized to give (R)-2-chloro-N-(5-chloro-6-((S)-2-hydroxypropoxy)pyridin-3-yl)-8-methyl-8-(trifluoromethyl)-7,8-dihydro-6H-pyrazolo[1,5-a]pyrrolo[2,3-e]pyrimidine-6-carboxamide (23.1 mg, 40% yield) as an off-white solid. The epimer of Example 73 can be prepared analogously using Method M1 isomer 1.

Example 73: ¹H NMR (400 MHz, Chloroform-d) δ: 9.38 (s, 1H), 8.04 (d, J=2.4 Hz, 1H), 7.99 (d, J=2.4 Hz, 1H), 6.75 (s, 1H), 6.38 (s, 1H), 4.53 (d, J=10.4 Hz, 1H), 4.38-4.42 (m, 1H), 4.21-4.27 (m, 2H), 4.02 (d, J=10.4 Hz, 1H), 2.07 (s, 3H), 1.30 (d, J=6.0 Hz, 3H). LC-MS: m/z 505 [M+H]⁺.

### Method X2

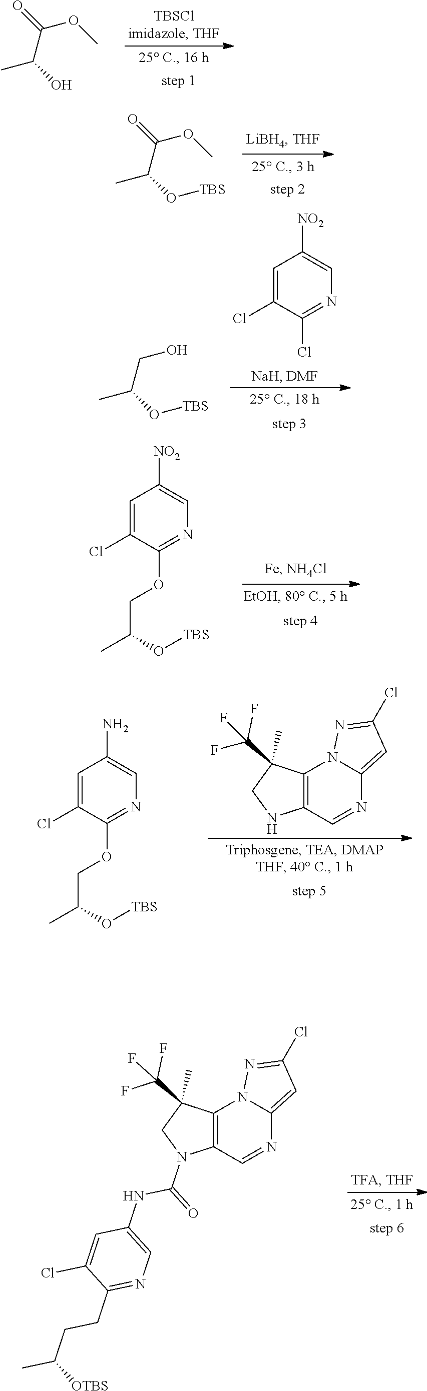

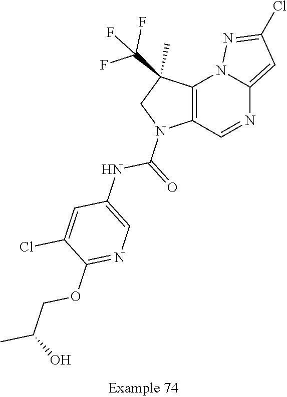 [SMILES: C[C@@H](O)COc1ncc(NC(=O)N2C[C@@](C)(C(F)(F)F)c3c2cnc2cc(Cl)nn32)cc1Cl]

### Example 74: (R)-2-chloro-N-(5-chloro-6-((R)-2-hydroxypropoxy)pyridin-3-yl)-8-methyl-8-(trifluormethyl)-7,8-dihydro-6H-pyrazolo[1,5-a]pyrrolo[2,3-e]pyrimidine-6-carboxamide

### Step 1: methyl (R)-2-((tert-butyldimethylsilyl)oxy)propanoate

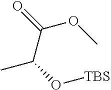

To a stirred solution of methyl (R)-2-hydroxypropanoate (5.0 g, 48.0 mmol) and imidazole (9.8 g, 143.9 mmol) in tetrahydrofuran (100 mL) were added tert-butylchlorodimethylsilane (8.6 g, 57.1 mmol) at 0° C. The reaction mixture was stirred at 25° C. for 16 h. The reaction mixture was quenched with water (50 mL). The resulting solution was extracted with ethyl acetate (2×100 mL). The combined organic layers were dried over anhydrous sodium sulfate and concentrated under vacuum to give methyl (R)-2-((tert-butyldimethylsilyl)oxy)propanoate (7.6 g, 65% yield) as a colorless oil. ¹H NMR (400 MHz, Chloroform-d) δ: 4.33 (q, J=6.8 Hz, 1H), 3.72 (s, 3H), 1.40 (d, J=6.8 Hz, 3H), 0.90 (s, 9H), 0.10 (s, 3H), 0.07 (s, 3H).

### Step 2: (R)-2-((tert-butyldimethylsilyl)oxy)propan-1-ol

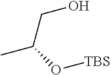

To a stirred solution of methyl (R)-2-((tert-butyldimethylsilyl)oxy)propanoate (5.0 g, 22.9 mmol) in tetrahydrofuran (30 mL) was added lithium tetrahydroborate (1.0 g, 45.9 mmol) at 0° C. The resulting mixture was stirred at 25° C. for 3 h. The reaction mixture was quenched with water (20 mL). The resulting solution was extracted with dichloromethane (3×20 mL). The combined organic layers were dried over anhydrous sodium sulfate and concentrated under vacuum. The residue was purified by column chromatography on silica gel using 80% petroleum ether and 20% ethyl acetate as eluent to afford (R)-2-((tert-butyldimethylsilyl)oxy)propan-1-ol (2.5 g, 57% yield) as a yellow oil. ¹H NMR (400 MHz, DMSO-d₆) δ: 4.60-4.57 (m, 1H), 3.75-3.79 (m, 1H), 3.31-3.35 (m, 1H), 3.15-3.21 (m, 1H), 1.09 (d, J=6.0 Hz, 3H), 0.90 (s, 9H), 0.08 (s, 6H).

### Step 3: (R)-2-(2-((tert-butyldimethylsilyl)oxy)propoxy)-3-chloro-5-nitropyridine

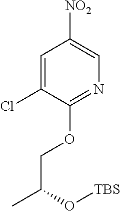

To a stirred solution of (R)-2-((tert-butyldimethylsilyl)oxy)propan-1-ol (1.0 g, 5.2 mmol) in N,N-Dimethylformamide (10 mL) was added NaH (231 mg, 5.9 mmol, 60% in mineral oil) at 0° C. The reaction mixture was stirred at 0° C. for 1 h. Then 2,3-dichloro-5-nitropyridine (1.2 g, 6.3 mmol) was added and the reaction mixture was stirred at 25° C. for 18 h. The reaction mixture was quenched with water (20 mL). The resulting solution was extracted with ethyl acetate (2×50 mL). The combined organic layers were dried over anhydrous sodium sulfate and concentrated under vacuum. The residue was purified by column chromatography on silica gel using 96% petroleum ether and 4% ethyl acetate as eluent to afford (R)-2-(2-((tert-butyldimethylsilyl)oxy)propoxy)-3-chloro-5-nitropyridine (0.8 g, 35% yield) as a yellow oil. ¹H NMR (400 MHz, DMSO-d₆) δ: 9.03 (d, J=2.8 Hz, 1H), 8.73 (d, J=2.4 Hz, 1H), 4.36-4.39 (m, 2H), 4.20-4.25 (m, 1H), 1.18 (d, J=6.4 Hz, 3H), 0.81 (s, 9H), 0.06 (s, 3H), 0.02 (s, 3H). LC-MS: m/z 347 [M+H]⁺.

### Step 4: (R)-6-(2-((tert-butyldimethylsilyl)oxy)propoxy)-5-chloropyridin-3-amine

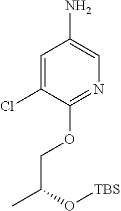

To a stirred mixture of (R)-2-(2-((tert-butyldimethylsilyl)oxy)propoxy)-3-chloro-5-nitropyridine (400 mg, 911.0 μmol) and NH₄Cl (97 mg, 1.8 mmol) in ethanol (15 mL) and water (5 mL) was added Fe (305 mg, 5.5 mmol). The reaction mixture was stirred at 80° C. for 5 h. The resulting mixture was filtered, and the filtrate was extracted with ethyl acetate (2×50 mL). The combined organic layers were dried over anhydrous sodium sulfate and concentrated under vacuum. The residue was purified by Prep-HPLC purification and the collected fractions were concentrated to afford (R)-6-(2-((tert-butyldimethylsilyl)oxy)propoxy)-5-chloropyridin-3-amine (110 mg, 30% yield) as a yellow oil. ¹H NMR (400 MHz, Chloroform-d) δ: 7.58 (d, J=2.4 Hz, 1H), 7.16 (d, J=2.4 Hz, 1H), 4.17-4.24 (m, 2H), 4.07-4.06 (m, 1H), 1.23 (d, J=5.6 Hz, 3H), 0.88 (s, 9H), 0.09 (s, 3H), 0.07 (s, 3H). LC-MS: m/z 317 [M+H]⁺.

### Step 5: (R)—N-(6-((R)-2-((tert-butyldimethylsilyl)oxy)propoxy)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide

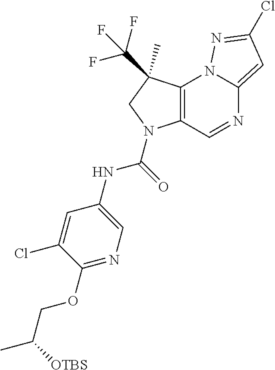 [SMILES: C[C@H](COc1ncc(NC(=O)N2C[C@@](C)(C(F)(F)F)c3c2cnc2cc(Cl)nn32)cc1Cl)O[Si](C)(C)C(C)(C)C]

To a stirred solution of (R)-6-(2-((tert-butyldimethylsilyl)oxy)propoxy)-5-chloropyridin-3-amine (60 mg, 189.3 μmol) in tetrahydrofuran (2 mL) were added triphosgene (42 mg, 141.5 μmol) and TEA (47 mg, 464.5 μmol). The resulting mixture was stirred at 40° C. for 1 h and then filtered. The filtrate was added to a solution of Method M1 isomer 2 (50 mg, 180.7 μmol) in tetrahydrofuran (2 mL). To this solution were added N,N-dimethylpyridin-4-amine (44 mg, 360.2 μmol) and TEA (183 mg, 1.8 mmol). The mixture was stirred at 40° C. for 1 h. The reaction mixture was quenched by the addition of methanol (2 mL) and the mixture was concentrated under vacuum. The residue was diluted with water (10 mL). The resulting mixture was extracted with ethyl acetate (2×20 mL). The combined organic layers were dried over anhydrous sodium sulfate and concentrated under vacuum. The residue was purified by column chromatography on silica gel using 60% petroleum ether and 40% ethyl acetate as eluent to afford (R)—N-(6-((R)-2-((tert-butyldimethylsilyl)oxy)propoxy)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide (33 mg, 28% yield) as an off-white solid. LC-MS: m/z 619 [M+H]⁺.

### Step 6: (R)-2-chloro-N-(5-chloro-6-((R)-2-hydroxypropoxy)pyridin-3-yl)-8-methyl-8-(trifluoromethyl)-7,8-dihydro-6H-pyrazolo[1,5-a]pyrrolo[2,3-e]pyrimidine-6-carboxamide

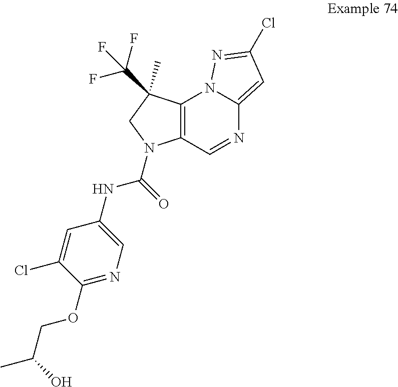 [SMILES: C[C@@H](O)COc1ncc(NC(=O)N2C[C@@](C)(C(F)(F)F)c3c2cnc2cc(Cl)nn32)cc1Cl]

To a stirred solution of (R)—N-(6-((R)-2-((tert-butyldimethylsilyl)oxy)propoxy)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide (32 mg, 51.6 μmol) in tetrahydrofuran (1 mL) was added TFA (0.5 mL). The reaction mixture was stirred at 25° C. for 1 h. The mixture was concentrated under vacuum. The residue was purified by Prep-HPLC purification and the collected fractions were lyophilized to give (R)-2-chloro-N-(5-chloro-6-((S)-2-hydroxypropoxy)pyridin-3-yl)-8-methyl-8-(trifluoromethyl)-7,8-dihydro-6H-pyrazolo[1,5-a]pyrrolo[2,3-e]pyrimidine-6-carboxamide (8.5 mg, 32% yield) as an off-white solid. The epimer of Example 74 can be prepared analogously using Method M1 isomer 1.

Example 74: ¹H NMR (400 MHz, Chloroform-d) δ: 9.38 (s, 1H), 8.06 (d, J=2.0 Hz, 1H), 8.00 (d, J=2.0 Hz, 1H), 6.75 (s, 1H), 6.42 (s, 1H), 4.54 (d, J=10.0 Hz, 1H), 4.38-4.44 (m, 1H), 4.22-4.29 (m, 2H), 4.02 (d, J=10.0 Hz, 1H), 2.07 (s, 3H), 1.30 (d, J=6.0 Hz, 3H). LC-MS: m/z 505 [M+H]⁺.

### Method Y2

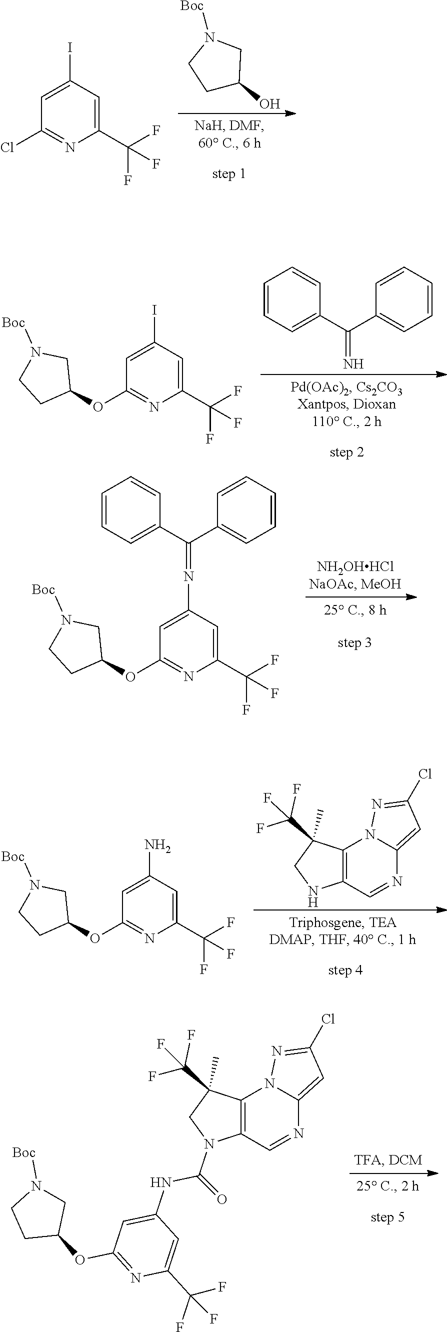

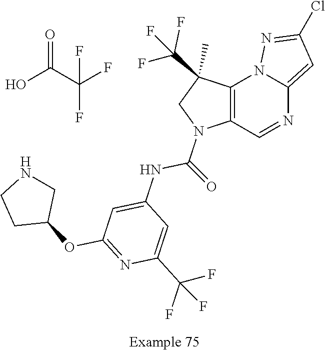

### Example 75: (R)-2-chloro-8-methyl-N-(2-(((S)-pyrrolidin-3-yl)oxy)-6-(trifluormethyl)pyridin-4-yl)-8-(trifluormethyl)-7,8-dihydro-6H-pyrazolo[1,5-a]pyrrolo[2,3-e]pyrimidine-6-carboxamide 2,2,2-trifluoroacetate

### Step 1: tert-butyl (S)-3-((4-iodo-6-(trifluoromethyl)pyridin-2-yl)oxy)pyrrolidine-1-carboxylate

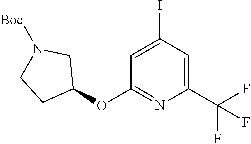 [SMILES: CC(C)(C)OC(=O)N1CC[C@H](Oc2cc(I)cc(C(F)(F)F)n2)C1]

To a stirred solution of tert-butyl (S)-3-hydroxypyrrolidine-1-carboxylate (1.0 g, 5.3 mmol) in N,N-dimethylformnamide (16 mL) was added NaH (256 mg, 6.4 mmol, 60% in mineral oil) at 0° C. in batches. The mixture was stirred at 0° C. for 20 mi. A solution of 2-chloro-4-iodo-6-(trifluoromethyl)pyridine (1.6 g, 5.3 mmol) in N,N-dimethylformamide (18 mL) was added into the mixture. The reaction mixture was stirred at 60° C. for 6 h. The mixture was cooled to 25° C. and quenched by the addition of water (100 mL). The resulting mixture was extracted with ethyl acetate (3×100 mL). The combined organic layers were dried over anhydrous sodium sulfate and concentrated under vacuum. The residue was purified by column chromatography on silica gel using 50% petroleum ether and 50% ethyl acetate as eluent to afford tert-butyl (S)-3-((4-iodo-6-(trifluoromethyl)pyridin-2-yl)oxy)pyrrolidine-1-carboxylate (533 mg, 22% yield) as a white solid. LC-MS: m/z 459 [M+H]⁺.

### Step 2: tert-butyl (S)-3-((4-((diphenylmethylene)amino)-6-(trifluoromethyl)pyridin-2-yl)oxy)pyrrolidine-1-carboxylate

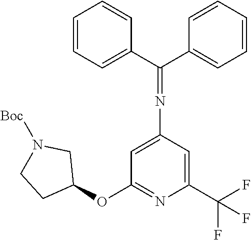 [SMILES: CC(C)(C)OC(=O)N1CC[C@H](Oc2cc(N=C(c3ccccc3)c3ccccc3)cc(C(F)(F)F)n2)C1]

To a stirred solution of tert-butyl (S)-3-((4-iodo-6-(trifluoromethyl)pyridin-2-yl)oxy)pyrrolidine-1-carboxylate (433 mg, 944.9 μmol) and diphenylmethanimine (171 mg, 944.9 μmol) in dioxane (17 mL) were added Pd₂(dba)₃ (197 mg, 190.3 μmol), Cs₂CO₃ (921 mg, 2.8 mmol) and Xantphos (164 mg, 283.5 μmol) under nitrogen atmosphere. The mixture was stirred at 110° C. for 2 h. The solution was then cooled to 25° C. and filtered. The filter cake was washed with ethyl acetate (10 mL). The filtrate was poured into water (50 mL) and extracted with ethyl acetate (3×50 mL). The combined organic layers were dried over anhydrous sodium sulfate and concentrated under vacuum. The residue was purified by column chromatography on silica gel using 80% petroleum ether and 20% ethyl acetate as eluent to afford tert-butyl (S)-3-((4-((diphenylmethylene)amino)-6-(trifluoromethyl)pyridin-2-yl)oxy)pyrrolidine-1-carboxylate (360 mg, 74% yield) as a yellow oil. LC-MS: m/z 512 [M+H]⁺.

### Step 3: tert-butyl (S)-3-((4-amino-6-(trifluoromethyl)pyridin-2-yl)oxy)pyrrolidine-1-carboxylate

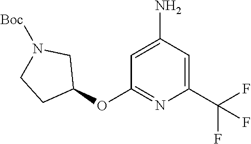 [SMILES: CC(C)(C)OC(=O)N1CC[C@H](Oc2cc(N)cc(C(F)(F)F)n2)C1]

To a stirred solution of tert-butyl (S)-3-((4-((diphenylmethylene)amino)-6-(trifluoromethyl)pyridin-2-yl)oxy)pyrrolidine-1-carboxylate (180 mg, 351.9 μmol) in methanol (10 mL) were added hydroxylamine hydrochloride (49 mg, 703.8 μmol) and sodium acetate (72 mg, 879.7 μmol). The mixture was stirred at 25° C. for 8 h. The resulting mixture was concentrated under vacuum. The residue was purified by Prep-TLC using 85% petroleum ether and 15% ethyl acetate as eluent to afford tert-butyl (S)-3-((4-amino-6-(trifluoromethyl)pyridin-2-yl)oxy)pyrrolidine-1-carboxylate (90 mg, 74% yield) as a colorless oil. LC-MS: m/z 348 [M+H]⁺.

### Step 4: tert-butyl (S)-3-((4-((R)-2-chloro-8-methyl-8-(trifluoromethyl)-7,8-dihydro-6H-pyrazolo[1,5-a]pyrrolo[2,3-e]pyrimidine-6-carboxamido)-6-(trifluoromethyl)pyridin-2-yl)oxy)pyrrolidine-1-carboxylate

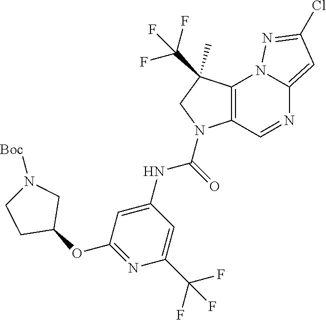 [SMILES: CC(C)(C)OC(=O)N1CC[C@H](Oc2cc(NC(=O)N3C[C@@](C)(C(F)(F)F)c4c3cnc3cc(Cl)nn43)cc(C(F)(F)F)n2)C1]

To a solution of tert-butyl (S)-3-((4-amino-6-(trifluoromethyl)pyridin-2-yl)oxy)pyrrolidine-1-carboxylate (90 mg, 259.3 μmol) in tetrahydrofuran (36 mL) were added triphosgene (46 mg, 155.6 μmol) and TEA (39 mg, 389.0 μmol). The mixture was stirred at 25° C. for 0.5 h and then filtered. The filtrate was added to a solution of Method M1 isomer 2 (50 mg, 181.4 μmol) in tetrahydrofuran (1 mL). To this solution were added TEA (261 mg, 2.6 mmol) and N,N-dimethylpyridin-4-amine (64 mg, 519.2 μmol). The mixture was stirred at 40° C. for 1 h. The mixture was poured into water (10 mL) and extracted with ethyl acetate (3×10 mL). The combined organic layers were washed with brine (20 mL), dried over anhydrous sodium sulfate and concentrated under vacuum. The residue was purified by Prep-TLC using 50% petroleum ether and 50% ethyl acetate as eluent to afford tert-butyl (S)-3-((4-((R)-2-chloro-8-methyl-8-(trifluoromethyl)-7,8-dihydro-6H-pyrazolo[1,5-a]pyrrolo[2,3-e]pyrimidine-6-carboxamido)-6-(trifluoromethyl)pyridin-2-yl)oxy)pyrrolidine-1-carboxylate (90 mg) as an off-white solid. LC-MS: m/z 650 [M+H]⁺.

### Step 5: (R)-2-chloro-8-methyl-N-(2-(((S)-pyrrolidin-3-yl)oxy)-6-(trifluoromethyl)pyridin-4-yl)-8-(trifluoromethyl)-7,8-dihydro-6H-pyrazolo[1,5-a]pyrrolo[2,3-e]pyrimidine-6-carboxamide 2,2,2-trifluoroacetate

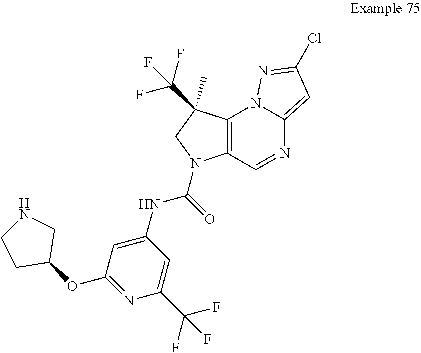 [SMILES: C[C@@]1(C(F)(F)F)CN(C(=O)Nc2cc(O[C@H]3CCNC3)nc(C(F)(F)F)c2)c2cnc3cc(Cl)nn3c21]

To a solution of tert-butyl (S)-3-((4-((R)-2-chloro-8-methyl-8-(trifluoromethyl)-7,8-dihydro-6H-pyrazolo[1,5-a]pyrrolo[2,3-e]pyrimidine-6-carboxamido)-6-(trifluoromethyl)pyridin-2-yl)oxy)pyrrolidine-1-carboxylate (80 mg, 123.1 μmol) in dichloromethane (5 mL) was added TFA (1 mL). The reaction mixture was stirred at 25° C. for 2 h. The mixture was concentrated under vacuum. The residue was submitted to Prep-HPLC purification and the collected fractions were lyophilized to afford Example 75 (21 mg, 25% yield) as a white solid. The epimer of Example 75 can be prepared analogously using Method M1 isomer 1.

Examples 75: ¹H NMR (400 MHz, DMSO-d₆) δ 9.74 (s, 1H), 9.32 (s, 1H), 7.67 (d, J=1.6 Hz, 1H), 7.50 (d, J=1.6 Hz, 1H), 7.08 (s, 1H), 5.57-5.60 (m, 1H), 4.83-4.86 (m, 1H), 4.26-4.28 (m, 1H), 3.40-3.50 (m, 4H), 2.27-2.33 (m, 1H), 2.16-2.18 (m, 1H), 1.97 (s, 3H). LC-MS: m/z 550 [M+H]⁺.

### Method Z2

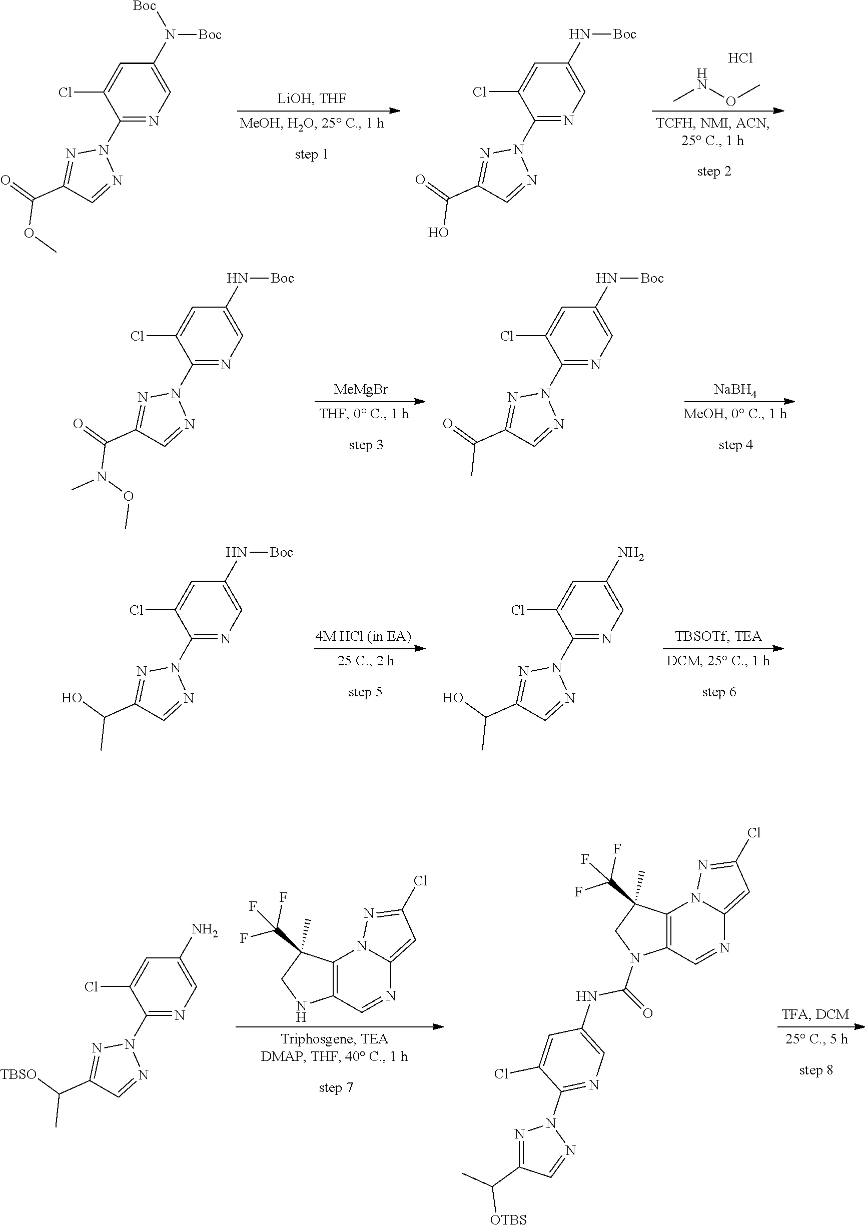

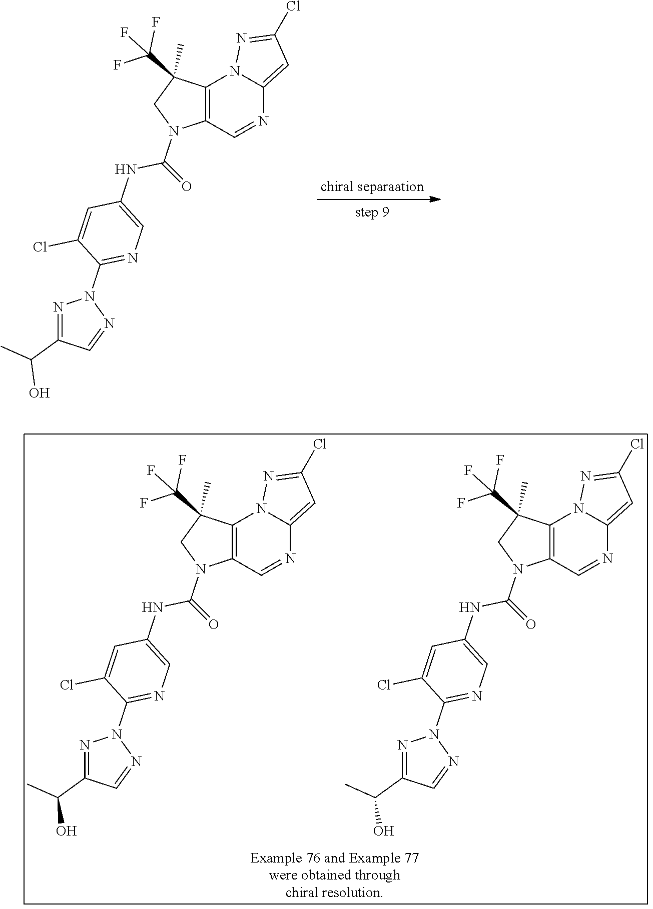 [SMILES: CC(O)c1cnn(-c2ncc(NC(=O)N3C[C@@](C)(C(F)(F)F)c4c3cnc3cc(Cl)nn43)cc2Cl)n1 ; C[C@H](O)c1cnn(-c2ncc(NC(=O)N3C[C@@](C)(C(F)(F)F)c4c3cnc3cc(Cl)nn43)cc2Cl)n1 ; C[C@@H](O)c1cnn(-c2ncc(NC(=O)N3C[C@@](C)(C(F)(F)F)c4c3cnc3cc(Cl)nn43)cc2Cl)n1]

### Examples 76 and 77: Single enantiomers obtained from a racemic mixture containing (R)-2-chloro-N-(5-chloro-6-(4-((S)-1-hydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(5-chloro-6-(4-((R)-1-hydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 2-(5-((tert-butoxycarbonyl)amino)-3-chloropyridin-2-yl)-2H-1,2,3-triazole-4-carboxylic acid

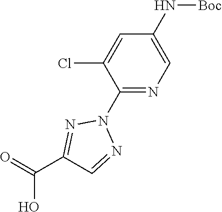 [SMILES: CC(C)(C)OC(=O)Nc1cnc(-n2ncc(C(=O)O)n2)c(Cl)c1]

To a stirred solution of methyl 2-(5-{bis[(tert-butoxy)carbonyl]amino}-3-chloropyridin-2-yl)-2H-1,2,3-triazole-4-carboxylate (1.8 g, 3.9 mmol; Method B3, step 3) in methanol (10 mL) and tetrahydrofran (20 mL) were added LiOH (190 mg, 7.9 mmol) in water (10 mL). The reaction was stirred at 25° C. for 1 h. The reaction was diluted with water (50 mL). The pH was adjusted to 4-5 with HCl (4 M). The mixture was extracted with ethyl acetate (2×100 mL). The organic layers were combined, dried over anhydrous sodium sulfate and concentrated under vacuum to give 2-(5-((tert-butoxycarbonyl)amino)-3-chloropyridin-2-yl)-2H-1,2,3-triazole-4-carboxylic acid (1.5 g, 94% yield) as a white solid. ¹H NMR (300 MHz, DMSO-d₆) δ: 13.02 (s, 1H), 10.26 (s, 1H), 8.59 (d, J=2.3 Hz, 1H), 8.55 (s, 1H), 8.35 (d, J=2.3 Hz, 1H), 1.52 (s, 9H). LC-MS: m/z 340 [M+H]⁺.

### Step 2: tert-butyl (5-chloro-6-(4-(methoxy(methyl)carbamoyl)-2H-1,2,3-triazol-2-yl)pyridin-3-yl)carbamate

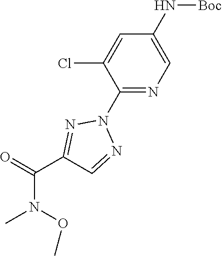 [SMILES: CON(C)C(=O)c1cnn(-c2ncc(NC(=O)OC(C)(C)C)cc2Cl)n1]

To a stirred solution of 2-(5-((tert-butoxycarbonyl)amino)-3-chloropyridin-2-yl)-2H-1,2,3-triazole-4-carboxylic acid (400 mg, 1.2 mmol) in acetonitrile were added N,O-dimethylhydroxylamine; hydrochloride (229 mg, 2.3 mmol), N-(chloro(dimethylamino)methylene)-N-methylmethanaminium hexafluorophosphate (991 mg, 3.5 mmol) and 1-methyl-1H-imidazole (676 mg, 8.2 mmol). The mixture was stirred at 25° C. for 1 h. The mixture was concentrated under vacuum. The residue was purified by column chromatography on silica gel using 75% petroleum ether and 25% ethyl acetate as eluent to afford tert-butyl (5-chloro-6-(4-(methoxy(methyl)carbamoyl)-2H-1,2,3-triazol-2-yl)pyridin-3-yl)carbamate (300 mg, 59% yield) as a white solid. ¹H NMR (400 MHz, DMSO-d₆) δ: 10.25 (s, 1H), 8.57 (d, J=2.3 Hz, 1H), 8.44 (s, 1H), 8.33 (d, J=2.3 Hz, 1H), 3.74 (s, 3H), 2.67 (s, 3H), 1.50 (s, 9H). LC-MS: m/z 383 [M+H]⁺.

### Step 3: tert-butyl (6-(4-acetyl-2H-1,2,3-triazol-2-yl)-5-chloropyridin-3-yl)carbamate

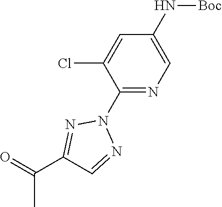 [SMILES: CC(=O)c1cnn(-c2ncc(NC(=O)OC(C)(C)C)cc2Cl)n1]

To a stirred solution of tert-butyl (5-chloro-6-(4-(methoxy(methyl)carbamoyl)-2H-1,2,3-triazol-2-yl)pyridin-3-yl)carbamate (280 mg, 731.1 μmol) in tetrahydrofuran (20 mL) was added methyl magnesium bromide (0.5 mL, 1.5 mmoL, 3 M in diethyl ether) dropwise at 0° C. under nitrogen atmosphere. The reaction was stirred at 0° C. for 1 h. The reaction was quenched with saturated aqueous NH₄Cl solution (50 mL). The resulted mixture was extracted with ethyl acetate (3×50 mL). The organic layers were combined, washed with brine, dried over anhydrous sodium sulfate and concentrated under vacuum. The residue was purified by column chromatography on silica gel using 70% petroleum ether and 30% ethyl acetate as eluent to afford tert-butyl (6-(4-acetyl-2H-1,2,3-triazol-2-yl)-5-chloropyridin-3-yl)carbamate (170 mg, 61% yield) as a white solid. ¹H NMR (400 MHz, DMSO-d₆) δ:10.27 (s, 1H), 8.48-8.67 (m, 2H), 8.34 (d, J=2.3 Hz, 1H), 2.59 (s, 3H), 1.50 (s, 9H). LC-MS: m/z 338 [M+H]⁺.

### Step 4: tert-butyl (5-chloro-6-(4-(1-hydroxyethyl)-2H-1,2,3-triazol-2-yl)pyridin-3-yl)carbamate

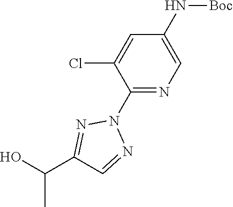 [SMILES: CC(O)c1cnn(-c2ncc(NC(=O)OC(C)(C)C)cc2Cl)n1]

To a stirred solution of tert-butyl (6-(4-acetyl-2H-1,2,3-triazol-2-yl)-5-chloropyridin-3-yl)carbamate (140 mg, 414.5 μmol) in methanol (4 mL) was added NaBH₄ (19 mg, 497.4 μmol) at 0° C. The reaction was stirred at 0° C. for 1 h. The solvent was removed under vacuum. The residue was purified by Prep-TLC using 90% dichloromethane and 10% methanol as eluent to afford tert-butyl (5-chloro-6-(4-(1-hydroxyethyl)-2H-1,2,3-triazol-2-yl)pyridin-3-yl)carbamate (140 mg, 94% yield) as a white solid. ¹H NMR (400 MHz, DMSO-d₆) δ: 10.16 (s, 1H), 8.53 (d, J=2.3 Hz, 1H), 8.28 (d, J=2.3 Hz, 1H), 7.98 (s, 1H), 5.50 (d, J=5.1 Hz, 1H), 4.88-4.94 (m, 1H), 1.49 (s, 9H), 1.43 (d, J=6.5 Hz, 3H). LC-MS: m/z 340 [M+H]⁺.

### Step 5: 1-(2-(5-amino-3-chloropyridin-2-yl)-2H-1,2,3-triazol-4-yl)ethan-1-ol

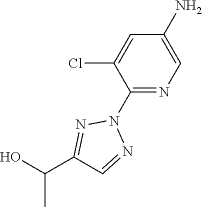 [SMILES: CC(O)c1cnn(-c2ncc(N)cc2Cl)n1]

To a stirred solution of tert-butyl (5-chloro-6-(4-(1-hydroxyethyl)-2H-1,2,3-triazol-2-yl)pyridin-3-yl)carbamate (100 mg, 220.2 μmol) in ethyl acetate (2 mL) was added HCl (2.2 mL, 4 M in ethyl acetate). The reaction was stirred at 25° C. for 2 h. The solvent was removed under vacuum. The residue was diluted with ethyl acetate (50 mL) and quenched by saturated aqueous NaHCO₃ solution (50 mL). The aqueous layer was extracted with ethyl acetate (2×50 mL). The combined organic layers were dried over anhydrous sodium sulfate and concentrated under vacuum. The residue was purified by Prep-TLC using 95% dichloromethane and 5% methanol as eluent to afford 1-(2-(5-amino-3-chloropyridin-2-yl)-2H-1,2,3-triazol-4-yl)ethan-1-ol (38 mg, 54% yield) as a colorless oil. ¹H NMR (300 MHz, DMSO-d₆) δ: 7.87 (s, 1H), 7.79 (d, J=2.5 Hz, 1H), 7.17 (d, J=2.5 Hz, 1H), 6.14 (s, 2H), 5.43 (s, 1H), 4.89 (s, 1H), 1.42 (d, J=6.5 Hz, 3H). LC-MS: m/z 240 [M+H]⁺.

### Step 6: 6-(4-(1-((tert-butyldimethylsilyl)oxy)ethyl)-2H-1,2,3-triazol-2-yl)-5-chloropyridin-3-amine

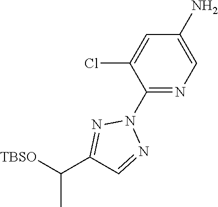 [SMILES: CC(O[Si](C)(C)C(C)(C)C)c1cnn(-c2ncc(N)cc2Cl)n1]

To a stirred solution of 1-(2-(5-amino-3-chloropyridin-2-yl)-2H-1,2,3-triazol-4-yl)ethan-1-ol (50 mg, 208.6 μmol) in dichloromethane (7 mL) was added trifluoromethanesulfonic acid tert-butyldimethylsilyl ester (72 mg, 271.2 μmol) and 2,6-dimethylpyridine (63 mg, 625.9 μmol) at 0° C. The reaction was stirred at 25° C. for 1 h. The solvent was removed under vacuum. The residue was purified by Prep-TLC using 50% petroleum ether and 50% methanol as eluent to afford 6-(4-(1-((tert-butyldimethylsilyl)oxy)ethyl)-2H-1,2,3-triazol-2-yl)-5-chloropyridin-3-amine (40 mg, 51% yield) as a white solid. LC-MS: m/z 354 [M+H]⁺.

### Step 7: (8R)—N-(6-(4-(1-((tert-butyldimethylsilyl)oxy)ethyl)-2H-1,2,3-triazol-2-yl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide

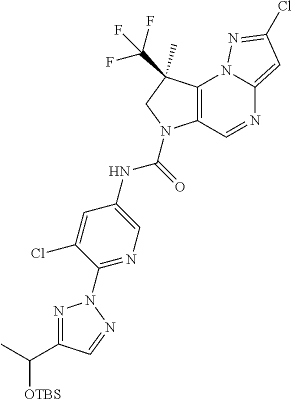 [SMILES: CC(O[Si](C)(C)C(C)(C)C)c1cnn(-c2ncc(NC(=O)N3C[C@@](C)(C(F)(F)F)c4c3cnc3cc(Cl)nn43)cc2Cl)n1]

To a stirred solution of Method M1 isomer 2 (31 mg, 113.0 μmol) in tetrahydrofuran (3 mL) were added TEA (17 mg, 169.5 μmol) and triphosgene (20 mg, 67.8 μmol). The mixture was stirred at 25° C. for 30 min and then filtered. The filtrate was added to a solution of 6-(((tert-butyldimethylsilyl)oxy)(cyclopropyl)methyl)-5-chloropyridin-3-amine (40 mg, 113.0 μmol) in tetrahydrofuran (1 mL). To this solution was then added N,N-dimethylpyridin-4-amine (28 mg, 226.0 μmol) and TEA (114 mg, 1.1 mmol). The reaction was stirred at 40° C. for 1 h. The solvent was removed under vacuum. The residue was purified by Prep-TLC using 97% dichloromethane and 3% methanol as eluent to afford (8R)—N-(6-(4-(1-((tert-butyldimethylsilyl)oxy)ethyl)-2H-1,2,3-triazol-2-yl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide (40 mg, 52% yield) as a white solid. ¹H NMR (300 MHz, Chloroform-d) δ: 9.43 (d, J=0.9 Hz, 1H), 8.49-8.63 (m, 1H), 8.42 (d, J=2.2 Hz, 1H), 7.90 (s, 1H), 6.88 (s, 1H), 6.79 (d, J=0.9 Hz, 1H), 5.21 (q, J=6.4 Hz, 1H), 4.62 (d, J=10.3 Hz, 1H), 4.08 (d, J=10.3 Hz, 1H), 2.09 (s, 3H), 1.59 (dd, J=6.4, 0.9 Hz, 3H), 0.94 (d, J=0.9 Hz, 9H), 0.07-0.15 (m, 6H). LC-MS: m/z 656 [M+H]⁺.

### Step 8: (8R)-2-chloro-N-(5-chloro-6-(4-(1-hydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

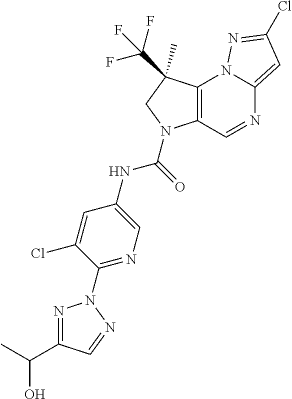 [SMILES: CC(O)c1cnn(-c2ncc(NC(=O)N3C[C@@](C)(C(F)(F)F)c4c3cnc3cc(Cl)nn43)cc2Cl)n1]

To a stirred solution of (8R)—N-(6-(4-(1-((tert-butyldimethylsilyl)oxy)ethyl)-2H-1,2,3-triazol-2-yl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide (30 mg, 45.7 μmol) in dichloromethane (12 mL) was added TFA (6 mL) at 25° C. The reaction was stirred at 25° C. for 5 h. The solvent was removed under vacuum. The residue was purified by Prep-TLC using 97% dichloromethane and 3% methanol as eluent to afford 24 mg of crude product. The crude product was purified by Prep-HPLC and the collected fractions were lyophilized to give (8R)-2-chloro-N-(5-chloro-6-(4-(1-hydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide (9.6 mg, 37% yield) as a light yellow solid. LC-MS: m/z 542 [M+H]⁺.

### Step 9: Separation of enantiomers to obtain (R)-2-chloro-N-(5-chloro-6-(4-((S)-1-hydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(5-chloro-6-(4-((R)-1-hydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

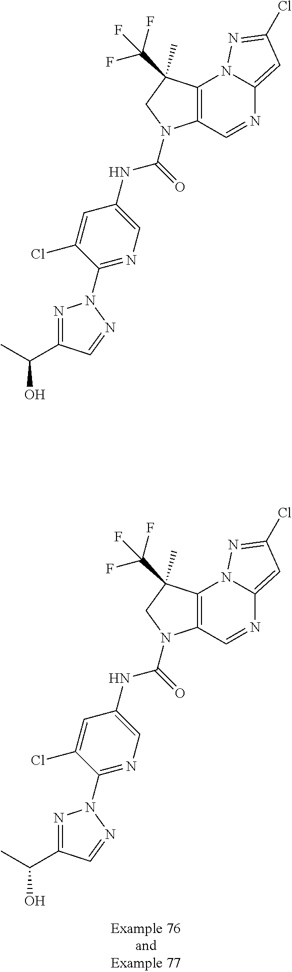 [SMILES: C[C@H](O)c1cnn(-c2ncc(NC(=O)N3C[C@@](C)(C(F)(F)F)c4c3cnc3cc(Cl)nn43)cc2Cl)n1 ; C[C@@H](O)c1cnn(-c2ncc(NC(=O)N3C[C@@](C)(C(F)(F)F)c4c3cnc3cc(Cl)nn43)cc2Cl)n1]

(8R)-2-chloro-N-(5-chloro-6-(4-(1-hydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide (9 mg, 16.6 μmol) was submitted to chiral HPLC purification Column: CHIRAL ART Cellulose-SB, 2×25 cm, 5 um; Mobile Phase A: MTBE (0.5% 2M NH₃-methanol)-HPLC, Mobile Phase B: IPA-HPLC; Flow rate: 20 mL/min; Gradient: 10 B to 10 B in 35 min; 220/254 nm; RT1: 25.605; RT2: 28.879; Injection Volume: 0.5 ml; Number Of Runs: 5. The first eluting isomer was concentrated and lyophilized to afford Example 76 (3.0 mg, 33% yield) as a white solid. The second eluting isomer was concentrated and lyophilized to afford Example 77 (3.0 mg, 33% yield) as a white solid. The corresponding enantiomers of Example 76 and Example 77 can be prepared analogously using Method M1 isomer 1. The enantiomers of Examples 76 and 77 can be prepared analogously using Method M1 isomer 1 in step 7.

Example 76: ¹H NMR (400 MHz, DMSO-d₆) δ: 9.67 (s, 1H), 9.34 (s, 1H), 8.72 (t, J=2.2 Hz, 1H), 8.48 (d, J=2.4 Hz, 1H), 8.01 (s, 1H), 7.06 (s, 1H), 5.51 (d, J=5.1 Hz, 1H), 4.90-5.04 (m, 1H), 4.84 (d, J=11.5 Hz, 1H), 4.29 (d, J=11.5 Hz, 1H), 1.97 (s, 3H), 1.45 (d, J=6.5 Hz, 3H). LC-MS: m/z 542 [M+H]⁺

Example 77: ¹H NMR (400 MHz, DMSO-d₆) δ: 9.67 (s, 1H), 9.34 (s, 1H), 8.72 (d, J=2.2 Hz, 1H), 8.48 (d, J=2.3 Hz, 1H), 8.01 (s, 1H), 7.06 (s, 1H), 5.51 (d, J=5.1 Hz, 1H), 4.88-4.98 (m, 1H), 4.84 (d, J=11.5 Hz, 1H), 4.29 (d, J=11.5 Hz, 1H), 1.97 (s, 3H), 1.45 (d, J=6.5 Hz, 3H). LC-MS: m/z 542 [M+H]⁺

### Method A3

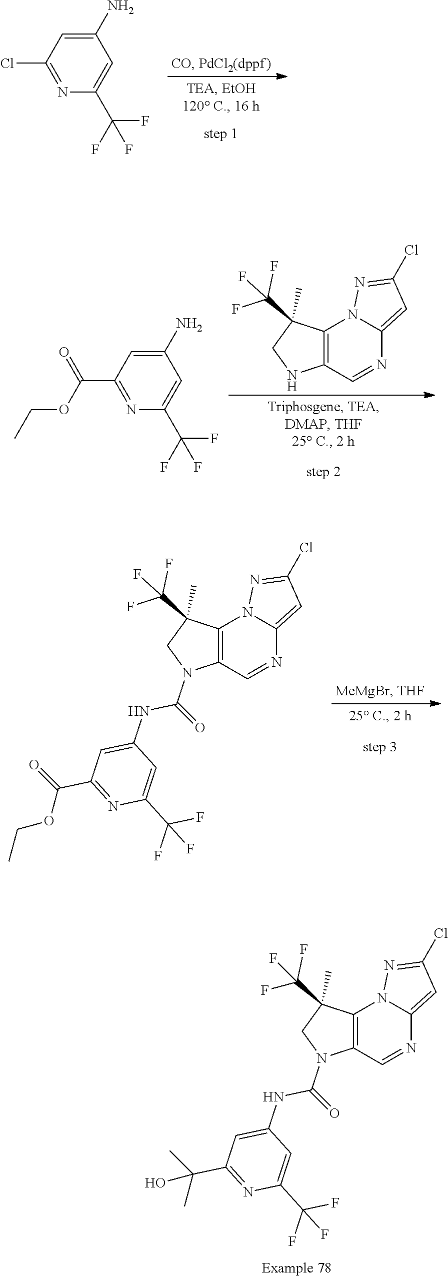 [SMILES: Nc1cc(Cl)nc(C(F)(F)F)c1 ; CCOC(=O)c1cc(N)cc(C(F)(F)F)n1 ; C[C@@]1(C(F)(F)F)CNc2cnc3cc(Cl)nn3c21 ; CCOC(=O)c1cc(NC(=O)N2C[C@@](C)(C(F)(F)F)c3c2cnc2cc(Cl)nn32)cc(C(F)(F)F)n1 ; CC(C)(O)c1cc(NC(=O)N2C[C@@](C)(C(F)(F)F)c3c2cnc2cc(Cl)nn32)cc(C(F)(F)F)n1]

### Example 78: (R)-2-chloro-N-(2-(2-hydroxypropan-2-yl)-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

### Step 1: ethyl 4-amino-6-(trifluoromethyl)picolinate

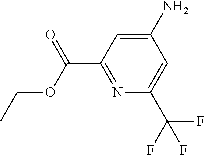 [SMILES: CCOC(=O)c1cc(N)cc(C(F)(F)F)n1]

To a mixture of 2-chloro-6-(trifluoromethyl) pyridin-4-amine (2.0 g, 10.2 mmol) in ethanol (30 mL) was added PdCl₂(dppf) (744 mg, 1.0 mmol) and TEA (3.1 g, 30.5 mmol). The resulting mixture was stirred at 120° C. for 16 h under carbon monoxide atmosphere. The reaction mixture was concentrated under vacuum. The residue was diluted with water (20 mL). The resulting mixture was extracted with ethyl acetate (3×10 mL). The combined organic layers were dried over anhydrous sodium sulfate and concentrated under vacuum. The residue was purified by column chromatography on silica gel using 50% petroleum ether and 50% ethyl acetate as eluent to afford ethyl 4-amino-6-(trifluoromethyl)picolinate (1.2 g, 48% yield) as a light-yellow solid. ¹H NMR (300 MHz, DMSO-d₆) δ: 7.41 (d, J=2.1 Hz, 1H), 7.07 (d, J=2.1 Hz, 1H), 6.96 (s, 2H), 4.33 (q, J=7.2 Hz, 2H), 1.32 (t, J=7.2 Hz, 3H). LC-MS: m/z 235 [M+H]⁺.

### Step 2: ethyl (R)-4-(2-chloro-8-methyl-8-(trifluoromethyl)-7,8-dihydro-6H-pyrazolo[1,5-a]pyrrolo[2,3-e]pyrimidine-6-carboxamido)-6-(trifluoromethyl)picolinate

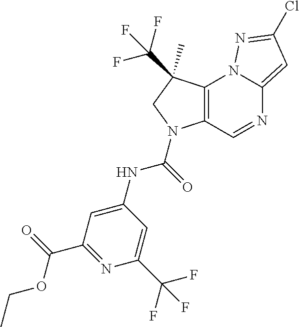 [SMILES: CCOC(=O)c1cc(NC(=O)N2C[C@@](C)(C(F)(F)F)c3c2cnc2cc(Cl)nn32)cc(C(F)(F)F)n1]

To a stirred solution of ethyl 4-amino-6-(trifluoromethyl)picolinate (44 mg, 180.7 μmol) in tetrahydrofuran (2 mL) were added triphosgene (32 mg, 108.4 μmol) and TEA (27 mg, 271.1 μmol). The resulting mixture was stirred at 25° C. for 0.5 h and then filtered. The filtrate was added to a solution of Method M1 isomer 2 (50 mg, 180.7 μmol) in tetrahydrofuran (2 mL). To this solution were added N,N-dimethylpyridin-4-amine (44 mg, 361.5 μmol) and TEA (183 mg, 1.8 mmol). The mixture was stirred at 25° C. for 2 h. The mixture was concentrated under vacuum. The residue was purified by Prep-HPLC and the collected fractions were lyophilized to afford ethyl (R)-4-(2-chloro-8-methyl-8-(trifluoromethyl)-7,8-dihydro-6H-pyrazolo[1,5-a]pyrrolo[2,3-e]pyrimidine-6-carboxamido)-6-(trifluoromethyl)picolinate (30 mg, 30% yield) as a white solid. ¹H NMR (400 MHz, DMSO-d₆) δ 9.96 (s, 1H), 9.34 (s, 1H), 8.51 (d, J=2.0 Hz, 1H), 8.36 (d, J=2.0 Hz, 1H), 7.07 (s, 1H), 4.87 (d, J=11.6 Hz, 1H), 4.40 (q, J=7.2 Hz, 2H), 4.29 (d, J=11.6 Hz, 1H), 1.97 (s, 3H), 1.35 (t, J=7.2 Hz, 3H). LC-MS: m/z 537 [M+H]⁺.

### Step 3: (R)-2-chloro-N-(2-(2-hydroxypropan-2-yl)-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

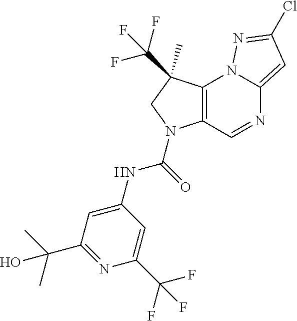 [SMILES: CC(C)(O)c1cc(NC(=O)N2C[C@@](C)(C(F)(F)F)c3c2cnc2cc(Cl)nn32)cc(C(F)(F)F)n1]

To a solution of ethyl (R)-4-(2-chloro-8-methyl-8-(trifluoromethyl)-7,8-dihydro-6H-pyrazolo[1,5-a]pyrrolo[2,3-e]pyrimidine-6-carboxamido)-6-(trifluoromethyl)picolinate (20 mg, 36.5 μmol) in THF (2 mL) was added methylmagnesium bromide (0.03 mL, 90 μmol, 3M in diethyl ether) at 0° C. The resulting mixture was stirred at 25° C. for 2 h. The reaction mixture was quenched with water (3 mL). The resulting solution was extracted with ethyl acetate (3×5 mL). The combined organic layers were dried over anhydrous sodium sulfate and concentrated under vacuum. The residue was purified by Prep-HPLC and the collected fractions were lyophilized to afford ethyl (R)-4-(2-chloro-8-methyl-8-(trifluoromethyl)-7,8-dihydro-6H-pyrazolo[1,5-a]pyrrolo[2,3-e]pyrimidine-6-carboxamido)-6-(trifluoromethyl)picolinate (1.5 mg, 6% yield) as an off-white solid. The corresponding enantiomer of Example 78 can be prepared analogously using Method M1 isomer 1.

Example 78: ¹H NMR (300 MHz, DMSO-d₆) δ: 9.75 (s, 1H), 9.36 (s, 1H), 8.15 (d, J=1.8 Hz, 1H), 8.05 (d, J=1.8 Hz, 1H), 7.09 (s, 1H), 5.46 (s, 1H), 4.91 (d, J=11.7 Hz, 1H), 4.29 (d, J=11.7 Hz, 1H), 1.97 (s, 3H), 1.46 (s, 6H). LC-MS: m/z 523 [M+H]⁺.

### Method B3

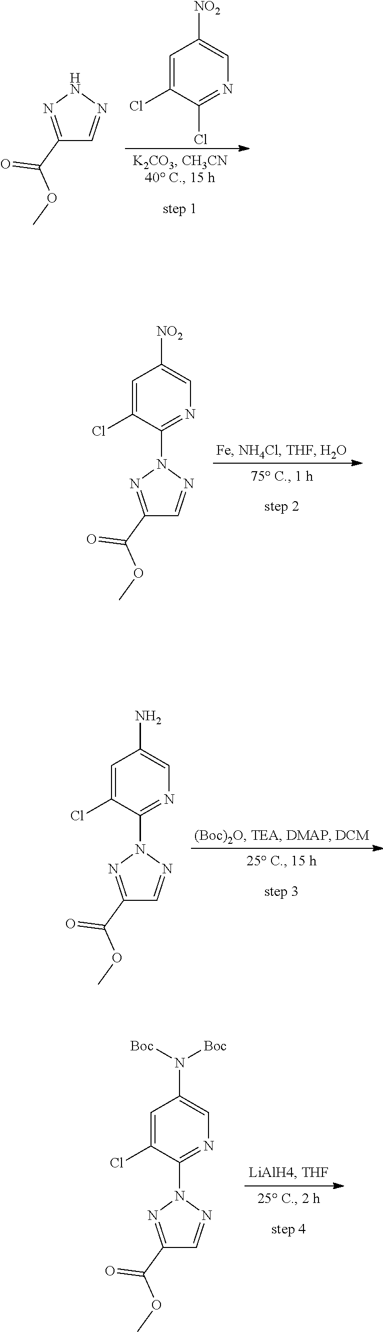

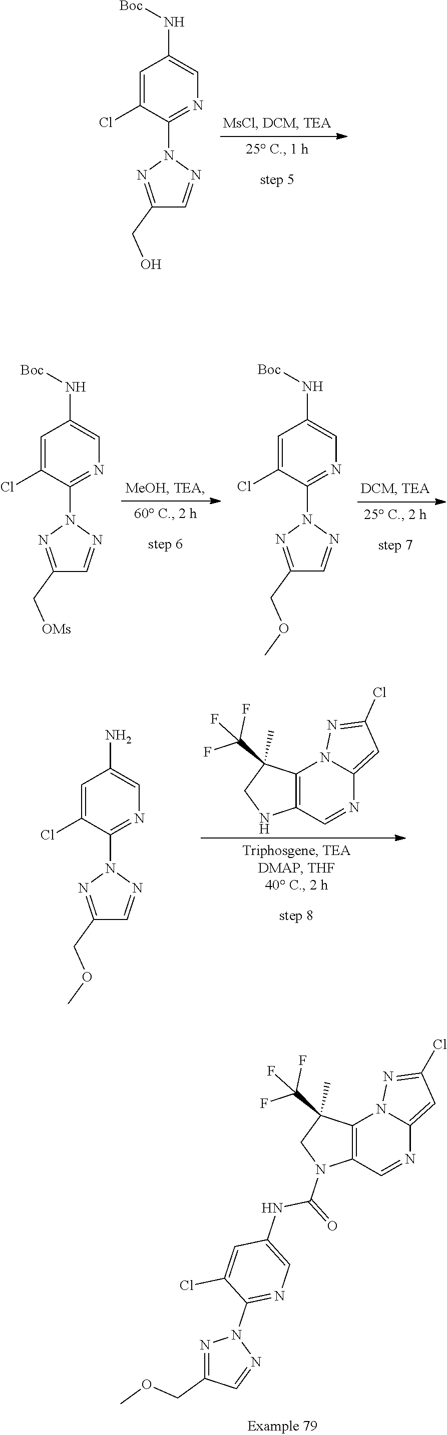 [SMILES: CC(C)(C)OC(=O)Nc1cnc(-n2ncc(CO)n2)c(Cl)c1 ; CC(C)(C)OC(=O)Nc1cnc(-n2ncc(COS(C)(=O)=O)n2)c(Cl)c1 ; COCc1cnn(-c2ncc(NC(=O)OC(C)(C)C)cc2Cl)n1 ; COCc1cnn(-c2ncc(N)cc2Cl)n1 ; C[C@@]1(C(F)(F)F)CNc2cnc3cc(Cl)nn3c21 ; COCc1cnn(-c2ncc(NC(=O)N3C[C@@](C)(C(F)(F)F)c4c3cnc3cc(Cl)nn43)cc2Cl)n1]

### Example 79: (R)-2-chloro-N-(5-chloro-6-(4-(methoxymethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: methyl 2-(3-chloro-5-nitropyridin-2-yl)-2H-1,2,3-triazole-4-carboxylate

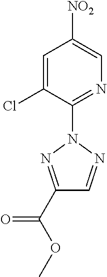 [SMILES: COC(=O)c1cnn(-c2ncc([N+](=O)[O-])cc2Cl)n1]

Into a 500 mL flask were placed methyl 2H-1,2,3-triazole-4-carboxylate (6.5 g, 51.5 mmol), acetonitrile (150 mL), 2,3-dichloro-5-nitropyridine (9.0 g, 46.9 mmol) and K₂CO₃ (8.4 g, 60.9 mmol). The reaction mixture was stirred at 40° C. for 15 h. The solid was filtered out. The filtrate was concentrated under vacuum. The residue was purified by Prep-HPLC purification and the collected fractions were concentrated to afford methyl 2-(3-chloro-5-nitropyridin-2-yl)-2H-1,2,3-triazole-4-carboxylate (4.9 g, 33.8% yield) as a white solid. ¹H NMR (300 MHz, Chloroform-d) δ: 9.36 (d, J=2.3 Hz, 1H), 8.83 (d, J=2.3 Hz, 1H), 8.44 (s, 1H), 4.05 (s, 3H). LC-MS: m/z 284 [M+H]⁺.

### Step 2: methyl 2-(5-amino-3-chloropyridin-2-yl)-2H-1,2,3-triazole-4-carboxylate

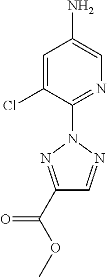 [SMILES: COC(=O)c1cnn(-c2ncc(N)cc2Cl)n1]

Into a 250 mL flask was placed methyl 2-(3-chloro-5-nitropyridin-2-yl)-2H-1,2,3-triazole-4-carboxylate (1.3 g, 4.6 mmol), tetrahydrofuran (20 mL), water (10 mL), NH₄Cl (1.2 g, 22.9 mmol), and Fe (1.3 g, 22.9 mmol). The mixture was stirred at 75° C. for 1 h. The reaction was cooled to 25° C. The solid was filtered out. The filtrate was concentrated under vacuum. The resulting mixture was diluted with water (100 mL). The resulting mixture was extracted with ethyl acetate (3×100 mL). The combined organic layers were dried over anhydrous sodium sulfate and concentrated under vacuum. The residue was purified by column chromatography on silica gel using 97% dichloromethane and 3% methanol as eluent to afford methyl 2-(5-amino-3-chloropyridin-2-yl)-2H-1,2,3-triazole-4-carboxylate (844 mg, 72.8% yield) as a white solid. ¹H NMR (400 MHz, Chloroform-d) δ: 8.30 (s, 1H), 7.90 (d, J=2.6 Hz, 1H), 7.16 (d, J=2.5 Hz, 1H), 3.98 (s, 3H). LC-MS: m/z 254 [M+H]⁺.

### Step 3: methyl 2-[5-[bis(tert-butoxycarbonyl)amino]-3-chloro-2-pyridyl]triazole-4-carboxylate

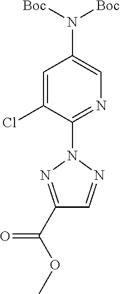 [SMILES: COC(=O)c1cnn(-c2ncc(N(C(=O)OC(C)(C)C)C(=O)OC(C)(C)C)cc2Cl)n1]

Into a 100 mL flask was placed methyl 2-(5-amino-3-chloropyridin-2-yl)-2H-1,2,3-triazole-4-carboxylate (844 mg, 3.3 mmol), dichloromethane (20 mL), TEA (673.4 mg, 6.7 mmol), and N,N-dimethylpyridin-4-amine (40.7 mg, 332.8 μmol). The reaction was cooled to 0° C. Then di-tert-butyl dicarbonate (1.5 g, 6.7 mmol) was added. The reaction was warmed to room temperature and stirred for 15 h. The resulting mixture was diluted with water (100 mL). The resulting mixture was extracted with dichloromethane (3×100 mL). The combined organic layers were dried over anhydrous sodium sulfate and concentrated under vacuum. The residue was purified by column chromatography on silica gel using 65% petroleum ether and 35% ethyl acetate as eluent to afford methyl 2-[5-[bis(tert-butoxycarbonyl)amino]-3-chloro-2-pyridyl]triazole-4-carboxylate (912 mg, 60.4% yield) as a white solid. ¹H NMR (400 MHz, Chloroform-d) δ: 8.36 (s, 1H), 8.35 (d, J=2.2 Hz, 1H), 7.83 (d, J=2.3 Hz, 1H), 4.00 (s, 3H), 1.45 (s, 18H). LC-MS: m/z 454 [M+H]⁺.

### Step 4: tert-butyl (5-chloro-6-(4-(hydroxymethyl)-2H-1,2,3-triazol-2-yl)pyridin-3-yl)carbamate

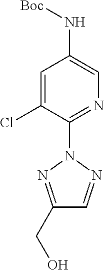 [SMILES: CC(C)(C)OC(=O)Nc1cnc(-n2ncc(CO)n2)c(Cl)c1]

Into a 100 mL flask was placed methyl 2-[5-[bis(tert-butoxycarbonyl)amino]-3-chloro-2-pyridyl]triazole-4-carboxylate (600 mg, 1.3 mmol) and tetrahydrofuran (20 mL). LiAlH₄ (75.3 mg, 1.9 mmol) was added at 0° C. in several portions. The mixture was stirred at 25° C. for 2 h. The reaction mixture was diluted with tetrahydrofuran (30 mL). The mixture was cooled to −30° C. and quenched by addition of water (75.3 mg), aqueous sodium hydroxide solution (75.3 mg, 10%) and water (75.3 mg). The solid was filtered out. The filtrate was concentrated under vacuum. The residue was purified by column chromatography on silica gel using 97% dichloromethane and 3% methanol as eluent to afford tert-butyl (5-chloro-6-(4-(hydroxymethyl)-2H-1,2,3-triazol-2-yl)pyridin-3-yl)carbamate (350 mg, 75.1% yield) as a white solid. ¹H NMR (400 MHz, DMSO-d₆) δ: 10.18 (s, 1H), 8.53 (d, J=2.3 Hz, 1H), 8.28 (d, J=2.3 Hz, 1H), 8.00 (s, 1H), 5.44 (t, J=5.8 Hz, 1H), 4.61 (d, J=5.8 Hz, 2H), 1.49 (s, 9H). LC-MS: m/z 326 [M+H]⁺.

### Step 5: (2-(5-((tert-butoxycarbonyl)amino)-3-chloropyridin-2-yl)-2H-1,2,3-triazol-4-yl)methyl methanesulfonate

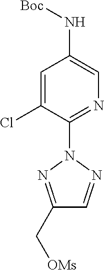 [SMILES: CC(C)(C)OC(=O)Nc1cnc(-n2ncc(COS(C)(=O)=O)n2)c(Cl)c1]

Into a 100 mL flask was placed tert-butyl (5-chloro-6-(4-(hydroxymethyl)-2H-1,2,3-triazol-2-yl)pyridin-3-yl)carbamate (260 mg, 798.2 μmol) and dichloromethane (10 mL). The reaction was cooled to 0° C. and methanesulfonyl chloride (137.1 mg, 1.20 mmol) and TEA (242.3 mg, 2.4 mmol) were added. The mixture was stirred at 25° C. for 1 h. The reaction mixture was concentrated under vacuum to give (2-(5-((tert-butoxycarbonyl)amino)-3-chloropyridin-2-yl)-2H-1,2,3-triazol-4-yl)methyl methanesulfonate (320 mg, crude) as a white oil. The product was used in the next step directly without further purification. LC-MS: m/z 404 [M+H]⁺.

### Step 6: tert-butyl (5-chloro-6-(4-(methoxymethyl)-2H-1,2,3-triazol-2-yl)pyridin-3-yl)carbamate

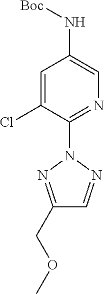 [SMILES: COCc1cnn(-c2ncc(NC(=O)OC(C)(C)C)cc2Cl)n1]

Into a 100 mL flask was placed (2-(5-((tert-butoxycarbonyl)amino)-3-chloropyridin-2-yl)-2H-1,2,3-triazol-4-yl)methyl (322 mg, 797 μmol), methanol (15 mL), and TEA (322.7 mg, 3.2 mmol). The mixture was stirred at 60° C. for 2 h. The reaction mixture was concentrated under vacuum. The residue was purified by column chromatography on silica gel using 80% petroleum ether and 20% ethyl acetate as eluent to afford tert-butyl (5-chloro-6-(4-(methoxymethyl)-2H-1,2,3-triazol-2-yl)pyridin-3-yl)carbamate (85 mg, 31.3% yield) as a colorless oil. ¹H NMR (400 MHz, DMSO-d₆) δ: ¹H NMR (400 MHz, DMSO-d₆) δ:10.17 (d, J=7.7 Hz, 1H), 8.55 (d, J=2.3 Hz, 1H), 8.30 (d, J=2.4 Hz, 1H), 8.09 (s, 1H), 4.57 (s, 2H), 3.32 (s, 3H), 1.50 (s, 9H). LC-MS: m/z 340 [M+H]⁺.

### Step 7: 5-chloro-6-(4-(methoxymethyl)-2H-1,2,3-triazol-2-yl)pyridin-3-amine

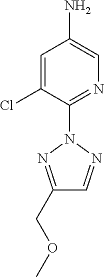 [SMILES: COCc1cnn(-c2ncc(N)cc2Cl)n1]

Into a 100 mL flask was placed tert-butyl (5-chloro-6-(4-(methoxymethyl)-2H-1,2,3-triazol-2-yl)pyridin-3-yl)carbamate (85 mg, 250.2 μmol), dichloromethane (4 mL), and TFA (1 mL). The reaction mixture was stirred at 25° C. for 2 h. The resulting solution was concentrated under vacuum. The residue was diluted with ethyl acetate (50 mL). The pH was adjusted to 7-8 with saturated aqueous NaHCO₃ solution. The resulting solution was extracted with ethyl acetate (2×50 mL). The combined organic layers were dried over anhydrous sodium sulfate and concentrated under vacuum. The residue was purified by column chromatography on silica gel using 98% dichloromethane and 2% methanol as eluent to afford 5-chloro-6-(4-(methoxymethyl)-2H-1,2,3-triazol-2-yl)pyridin-3-amine (25 mg, 41.2% yield) as a yellow solid. ¹H NMR (300 MHz, DMSO-d₆) δ: 8.00 (s, 1H), 7.81 (d, J=2.5 Hz, 1H), 7.20 (d, J=2.5 Hz, 1H), 6.17 (s, 2H), 4.55 (s, 2H), 3.31 (s, 3H). LC-MS: m/z 240 [M+H]⁺.

### Step 8: (R)-2-chloro-N-(5-chloro-6-(4-(methoxymethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

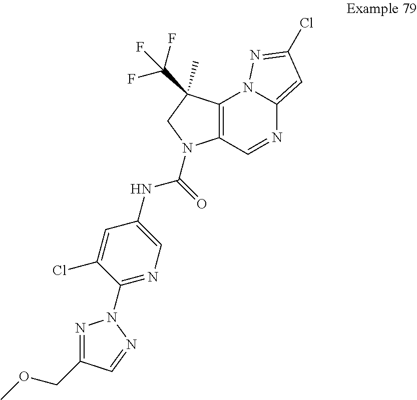 [SMILES: COCc1cnn(-c2ncc(NC(=O)N3C[C@@](C)(C(F)(F)F)c4c3cnc3cc(Cl)nn43)cc2Cl)n1]

To a stirred solution of Method isomer 2 (16.5 mg, 59.6 μmol) in tetrahydrofuran (3 mL) was added triphosgene (10.6 mg, 35.8 μmol) and TEA (9.1 mg, 89.4 μmol). The resulting mixture was stirred for 0.5 h at 28° C. and then filtered. The resulting filtrate was added to a solution of 5-chloro-6-(4-(methoxymethyl)-2H-1,2,3-triazol-2-yl)pyridin-3-amine (10 mg, 41.7 μmol) in tetrahydrofuran (1 mL). To this solution was then added TEA (60.3 mg, 596.1 μmol) and N,N-dimethylpyridin-4-amine (728.2 ug, 5.9 μmol). The mixture was stirred at 40° C. for 2 h. The solvent was concentrated under vacuum. The residue was purified by Prep-TLC using 96% dichloromethane and 4% methanol as eluent to afford 30 mg of crude product. The afforded crude was purified by Prep-HPLC purification and the collected fractions were lyophilized to give (R)-2-chloro-N-(5-chloro-6-(4-(methoxymethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide (3 mg, 15.0% yield) as a white solid. The enantiomer of Example 79 can be prepared analogously using Method M1 isomer 1.

Example 79: ¹H NMR (400 MHz, DMSO-d₆) δ: 9.70 (s, 1H), 9.35 (s, 1H), 8.72 (s, 1H), 8.49 (d, J=2.3 Hz, 1H), 8.12 (s, 1H), 7.06 (s, 1H), 4.84 (d, J=11.5 Hz, 1H), 4.58 (s, 2H), 4.28 (d, J=11.5 Hz, 1H), 3.32 (s, 3H), 1.97 (s, 3H). LC-MS: m/z 542 [M+H]⁺.

### Method C3

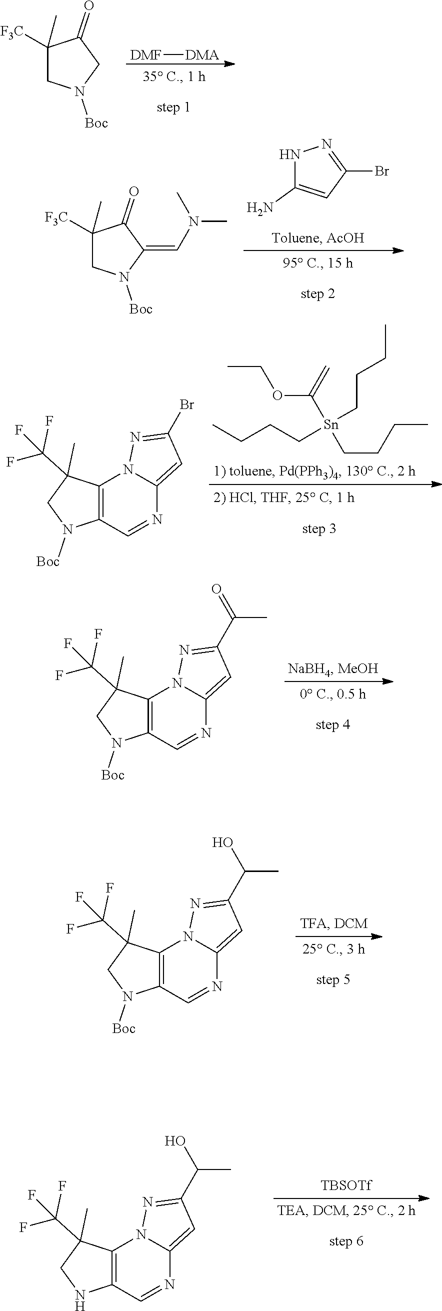

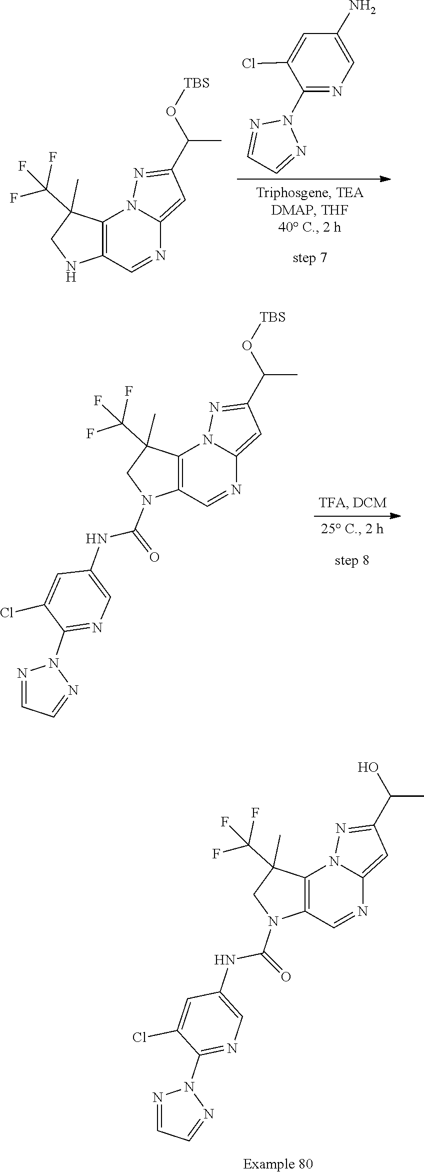

### Example 80: N-(5-chloro-6-(2H-1,2,3-triazol-2-yl)pyridin-3-yl)-2-(1-hydroxyethyl)-8-methyl-8-(trifluoromethyl)-7,8-dihydro-6H-pyrazolo[1,5-a]pyrrolo[2,3-e]pyrimidine-6-carboxamide

### Step 1: tert-butyl (E)-2-((dimethylamino)methylene)-4-methyl-3-oxo-4-(trifluoromethyl)pyrrolidine-1-carboxylate

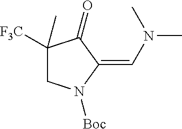 [SMILES: CN(C)/C=C1\C(=O)C(C)(C(F)(F)F)CN1C(=O)OC(C)(C)C]

Into a 100 ml flask was placed tert-butyl 3-methyl-4-oxo-3-(trifluoromethyl)pyrrolidine-1-carboxylate (1.0 g, 3.7 mmol; Method K1, step 7), DMF-DMA (10 mL). The reaction was stirred at 35° C. for 1 h. The reaction mixture was concentrated under vacuum to give tert-butyl (E)-2-((dimethylamino)methylene)-4-methyl-3-oxo-4-(trifluoromethyl)pyrrolidine-1-carboxylate (1.3 g, crude) as a yellow oil. The product was used in the next step directly without further purification. LC-MS: m/z 323 [M+H]⁺.

### Step 2: tert-butyl 2-bromo-8-methyl-8-(trifluoromethyl)-7,8-dihydro-6H-pyrazolo[1,5-a]pyrrolo[2,3-e]pyrimidine-6-carboxylate

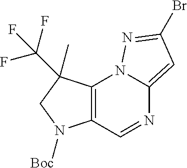 [SMILES: CC(C)(C)OC(=O)N1CC(C)(C(F)(F)F)c2c1cnc1cc(Br)nn21]

Into a 100 mL flask was placed tert-butyl (E)-2-((dimethylamino)methylene)-4-methyl-3-oxo-4-(trifluoromethyl)pyrrolidine-1-carboxylate (1.1 g, 3.4 mmol), toluene (15 mL), 3-bromo-1H-pyrazol-5-amine (527.2 mg, 3.3 mmol) and acetic acid (1.5 mL). The mixture was stirred at 95° C. for 15 h. The resulting solution was concentrated under vacuum. The residue was diluted with ethyl acetate (100 mL). The pH was adjusted to 7-8 with saturated aqueous NaHCO₃ solution. The resulting solution was extracted with ethyl acetate (2×100 mL). The organic layers were combined, dried over anhydrous sodium sulfate and concentrated under vacuum. The residue was purified by column chromatography on silica gel using 80% petroleum ether and 20% ethyl acetate as eluent to afford tert-butyl 2-bromo-8-methyl-8-(trifluoromethyl)-7,8-dihydro-6H-pyrazolo[1,5-a]pyrrolo[2,3-e]pyrimidine-6-carboxylate (408 mg, 28.6% yield) as a yellow solid. ¹H NMR (400 MHz, DMSO-d₆) δ: 8.82-9.13 (m, 1H), 7.09 (s, 1H), 4.32-4.35 (m, 1H), 3.99-4.03 (m, 1H), 1.90 (s, 3H), 1.52 (s, 9H). LC-MS: m/z 421 [M+H]⁺.

### Step 3: tert-butyl 2-acetyl-8-methyl-8-(trifluoromethyl)-7,8-dihydro-6H-pyrazolo[1,5-a]pyrrolo[2,3-e]pyrimidine-6-carboxylate

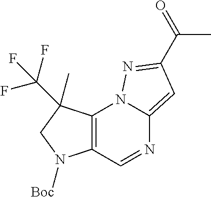 [SMILES: CC(=O)c1cc2ncc3c(n2n1)C(C)(C(F)(F)F)CN3C(=O)OC(C)(C)C]

Into a 100 mL flask was placed tert-butyl 2-bromo-8-methyl-8-(trifluoromethyl)-7,8-dihydro-6H-pyrazolo[1,5-a]pyrrolo[2,3-e]pyrimidine-6-carboxylate (364 mg, 864.2 μmol), toluene (10 mL), tributyl(1-ethoxyvinyl)stannane (343.3 mg, 950.6 μmol), Pd(PPh₃)₄ (99.9 mg, 86.4 μmol) under nitrogen atmosphere. The mixture was stirred at 130° C. for 2 h. The reaction mixture was concentrated under vacuum. The resulting residue was dissolved in tetrahydrofuran (5 mL) and HCl (5 mL, 2 M in H₂O) was added. The mixture was stirred at 25° C. for 1 h and then concentrated under vacuum. The residue was diluted with ethyl acetate (100 mL). The pH was adjusted to 7-8 with saturated aqueous NaHCO₃ solution. The resulting solution was extracted with ethyl acetate (2×100 mL). The organic layers were combined, dried over anhydrous sodium sulfate and concentrated under vacuum. The residue was purified by column chromatography on silica gel using 87% petroleum ether and 13% ethyl acetate as eluent to afford tert-butyl 2-acetyl-8-methyl-8-(trifluoromethyl)-7,8-dihydro-6H-pyrazolo[1,5-a]pyrrolo[2,3-e]pyrimidine-6-carboxylate (240 mg, 72.1% yield) as a white solid. ¹H NMR (400 MHz, DMSO-d₆) δ: 8.91-9.21 (m, 1H), 7.29 (s, 1H), 4.37 (d, J=12.4 Hz, 1H), 4.07 (d, J=12.4 Hz, 1H), 2.61 (s, 3H), 1.97 (s, 3H), 1.53 (s, 9H). LC-MS: m/z 385 [M+H]⁺.

### Step 4: tert-butyl 2-(1-hydroxyethyl)-8-methyl-8-(trifluoromethyl)-7,8-dihydro-6H-pyrazolo[1,5-a]pyrrolo[2,3-e]pyrimidine-6-carboxylate

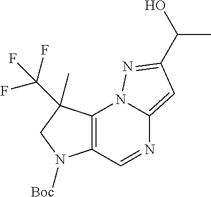 [SMILES: CC(O)c1cc2ncc3c(n2n1)C(C)(C(F)(F)F)CN3C(=O)OC(C)(C)C]

Into a 100 mL flask was placed tert-butyl 2-acetyl-8-methyl-8-(trifluoromethyl)-7,8-dihydro-6H-pyrazolo[1,5-a]pyrrolo[2,3-e]pyrimidine-6-carboxylate (197 mg, 512.6 μmol), methanol (10 mL). NaBH₄ (23.3 mg, 615.1 μmol) was added 0° C. The mixture was stirred at 0° C. for 0.5 h. The reaction was quenched by the addition of ice water (5 mL). The reaction mixture was concentrated under vacuum. The residue was purified by column chromatography on silica gel using 50% petroleum ether and 50% ethyl acetate as eluent to afford tert-butyl 2-(1-hydroxyethyl)-8-methyl-8-(trifluoromethyl)-7,8-dihydro-6H-pyrazolo[1,5-a]pyrrolo[2,3-e]pyrimidine-6-carboxylate (197 mg, 98.4% yield) as a yellow solid. ¹H NMR (400 MHz, DMSO-d) δ: 8.74-9.06 (m, 1H), 6.74 (d, J=1.4 Hz, 1H), 5.41 (t, J=5.0 Hz, 1H), 4.83-4.93 (m, 1H), 4.32 (d, J=12.0 Hz, 1H), 4.01 (q, J=7.1 Hz, 1H), 1.92 (s, 3H), 1.52 (s, 9H), 1.41-1.44 (m, 3H). LC-MS: m/z 387 [M+H]⁺.

### Step 5: 1-(8-methyl-8-(trifluoromethyl)-7,8-dihydro-6H-pyrazolo[1,5-a]pyrrolo[2,3-e]pyrimidin-2-yl)ethan-1-ol

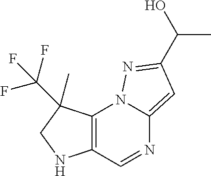 [SMILES: CC(O)c1cc2ncc3c(n2n1)C(C)(C(F)(F)F)CN3]

Into a 50 mL flask was placed tert-butyl 2-(1-hydroxyethyl)-8-methyl-8-(trifluoromethyl)-7,8-dihydro-6H-pyrazolo[1,5-a]pyrrolo[2,3-e]pyrimidine-6-carboxylate (50 mg, 129.4 μmol), dichloromethane (4 mL), and TFA (1 mL). The mixture was stirred at 25° C. for 3.0 h and then concentrated under vacuum. The residue was diluted with ethyl acetate (50 mL). The pH was adjusted to 7-8 with saturated aqueous NaHCO₃ solution. The resulting solution was extracted with ethyl acetate (2×50 mL). The combined organic layers were dried over anhydrous sodium sulfate and concentrated under vacuum. The residue was purified by column chromatography on silica gel using 96% dichloromethane and 4% methanol as eluent to afford 1-(8-methyl-8-(trifluoromethyl)-7,8-dihydro-6H-pyrazolo[1,5-a]pyrrolo[2,3-e]pyrimidin-2-yl)ethan-1-ol (23 mg, 62.1% yield) as a yellow solid. ¹H NMR (300 MHz, DMSO-d₆) δ: 8.26 (s, 1H), 6.60 (s, 1H), 5.76 (t, J=3.0 Hz, 1H), 5.23-5.27 (m, 1H), 4.82-4.90 (m, 1H), 3.88 (dd, J=11.4, 2.4 Hz, 1H), 3.55 (dd, J=11.4, 4.1 Hz, 1H), 1.83 (s, 3H), 1.44 (dd, J=6.5, 1.5 Hz, 3H). LC-MS: m/z 287 [M+H]⁺.

### Step 6: 2-(1-((tert-butyldimethylsilyl)oxy)ethyl)-8-methyl-8-(trifluoromethyl)-7,8-dihydro-6H-pyrazolo[1,5-a]pyrrolo[2,3-e]pyrimidine

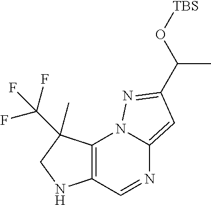

Into a 50 mL flask was placed 1-(8-methyl-8-(trifluoromethyl)-7,8-dihydro-6H-pyrazolo[1,5-a]pyrrolo[2,3-e]pyrimidin-2-yl)ethan-1-ol (112 mg, 391.3 μmol), dichloromethane (7 mL), TEA (118.8 mg, 1.2 mmol) and tert-butyldimethylsilyl trifluoromethanesulfonate (134.5 mg, 508.6 μmol). The mixture was stirred at 25° C. for 2 h and then concentrated under vacuum. The residue was purified by column chromatography on silica gel using 95% dichloromethane and 5% methanol as eluent to afford 2-(1-((tert-butyldimethylsilyl)oxy)ethyl)-8-methyl-8-(trifluoromethyl)-7,8-dihydro-6H-pyrazolo[1,5-a]pyrrolo[2,3-e]pyrimidine (108 mg, 68.7% yield) as a yellow oil. LC-MS: m/z 401 [M+H]⁺.

### Step 7: 2-(1-((tert-butyldimethylsilyl)oxy)ethyl)-N-(5-chloro-6-(2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

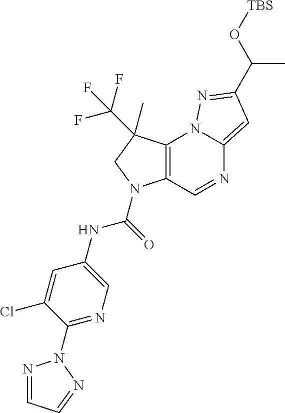

To a stirred solution of 5-chloro-6-(2H-1,2,3-triazol-2-yl)pyridin-3-amine (26.4 mg, 134.8 μmol) in tetrahydrofuran (5 mL) was added triphosgene (20.0 mg, 67.4 μmol) and TEA (17.1 mg, 168.5 μmol). The resulting mixture was stirred at 28° C. for 0.5 h and then filtered. The resulting filtrate was added to a solution of 2-(1-((tert-butyldimethylsilyl)oxy)ethyl)-8-methyl-8-(trifluoromethyl)-7,8-dihydro-6H-pyrazolo[1,5-a]pyrrolo[2,3-e]pyrimidine (45 mg, 112.4 μmol) in tetrahydrofuran (1.5 mL). To this solution was then added TEA (113.7 mg, 1.1 mmol) and N,N-dimethylpyridin-4-amine (27.45 mg, 224.71 μmol). The mixture was stirred at 40° C. for 2 h. The solvent was concentrated under vacuum. The residue was purified by Prep-TLC using 97% dichloromethane and 3% methanol as eluent to afford 2-(1-((tert-butyldimethylsilyl)oxy)ethyl)-N-(5-chloro-6-(2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide (40 mg, 57.1% yield) as a yellow solid. LC-MS: m/z 622 [M+H]⁺.

### Step 8: N-(5-chloro-6-(2H-1,2,3-triazol-2-yl)pyridin-3-yl)-2-(1-hydroxyethyl)-8-methyl-8-(trifluoromethyl)-7,8-dihydro-6H-pyrazolo[1,5-a]pyrrolo[2,3-e]pyrimidine-6-carboxamide

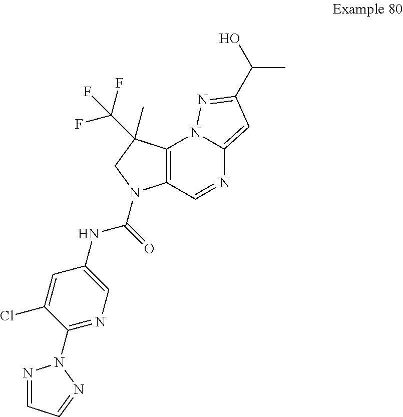 [SMILES: CC(O)c1cc2ncc3c(n2n1)C(C)(C(F)(F)F)CN3C(=O)Nc1cnc(-n2nccn2)c(Cl)c1]

Into a 100 mL flask was placed 2-(1-((tert-butyldimethylsilyl)oxy)ethyl)-N-(5-chloro-6-(2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide (40 mg, 64.3 μmol), dichloromethane (1.2 mL), and TFA (0.4 mL). The reaction mixture was stirred at 25° C. for 2 h and then concentrated under vacuum. The residue was diluted with ethyl acetate (30 mL). The pH was adjusted to 7-8 with saturated aqueous NaHCO₃ solution. The resulting solution was extracted with ethyl acetate (2×30 mL). The organic layers were combined, dried over anhydrous sodium sulfate and concentrated under vacuum. The residue was purified by Prep-TLC using 97% dichloromethane and 3% methanol as eluent to afford 28 mg crude. The afforded crude was purified by Prep-HPLC purification and the collected fractions were lyophilized to give N-(5-chloro-6-(2H-1,2,3-triazol-2-yl)pyridin-3-yl)-2-(1-hydroxyethyl)-8-methyl-8-(trifluoromethyl)-7,8-dihydro-6H-pyrazolo[1,5-a]pyrrolo[2,3-e]pyrimidine-6-carboxamide (8.9 mg, 26.9% yield) as a white solid.

Example 80: ¹H NMR (400 MHz, DMSO-d₆) δ: ¹H NMR (400 MHz, DMSO-d₆) δ: 9.27 (s, 1H), 8.78 (s, 1H), 8.54 (s, 1H), 8.18 (s, 2H), 6.79 (s, 1H), 5.42-5.44 (m, 1H), 4.84-4.94 (m, 2H), 4.31 (d, J=10.8 Hz, 1H), 2.02 (s, 3H), 1.48 (d, J=6.0 Hz, 3H). LC-MS: m/z 508 [M+H]⁺.

### Method D3

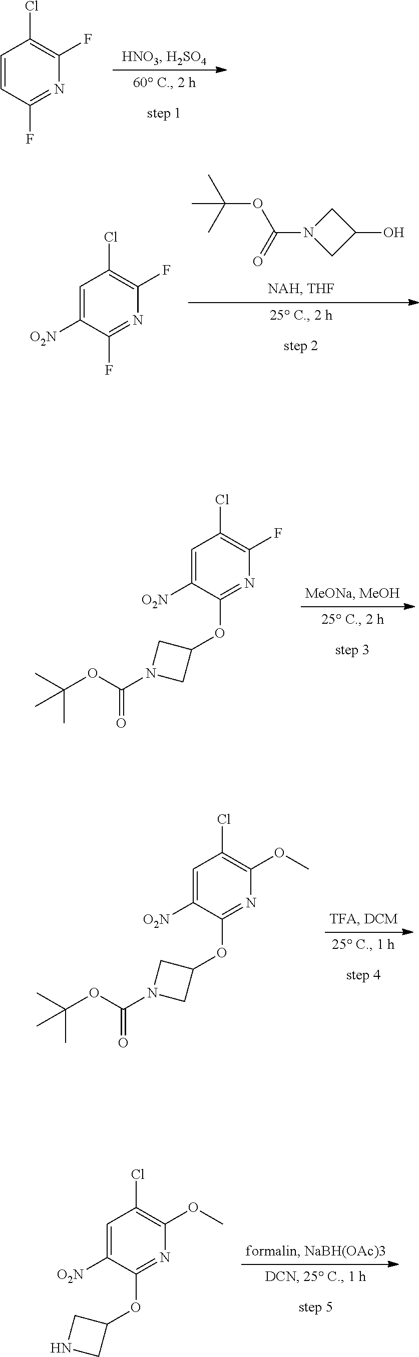 [SMILES: Fc1ccc(Cl)c(F)n1 ; O=[N+]([O-])c1cc(Cl)c(F)nc1F ; CC(C)(C)OC(=O)N1CC(O)C1 ; CC(C)(C)OC(=O)N1CC(Oc2nc(F)c(Cl)cc2[N+](=O)[O-])C1 ; COc1nc(OC2CN(C(=O)OC(C)(C)C)C2)c([N+](=O)[O-])cc1Cl ; COc1nc(OC2CNC2)c([N+](=O)[O-])cc1Cl]

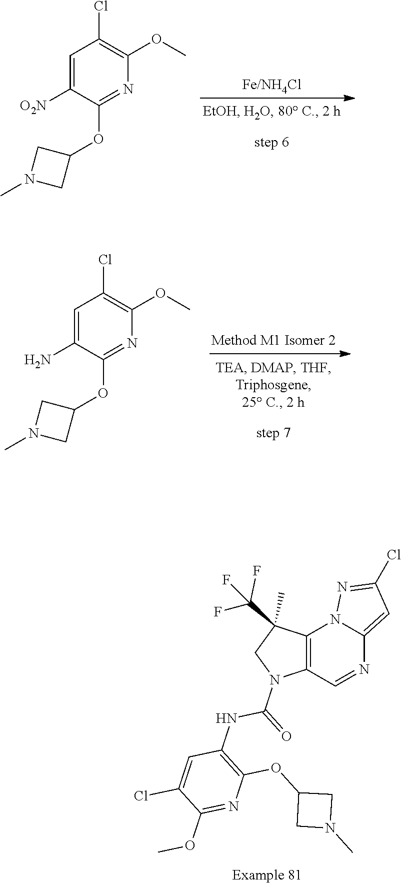

### Examples 81: (R)-2-chloro-N-(5-chloro-6-methoxy-2-((1-methylazetidin-3-yl)oxy)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 3-chloro-2,6-difluoro-5-nitropyridine

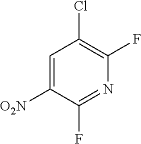 [SMILES: O=[N+]([O-])c1cc(Cl)c(F)nc1F]

To a solution of 3-chloro-2,6-difluoro-pyridine (4.9 g, 32.7 mmol) in fuming nitric acid (40 mL) was added sulfuric acid (30 mL) dropwise. The resulting mixture was stirred at 60° C. for 2 h. The reaction mixture was cooled to 25° C., and poured into crushed ice (200 g). The solution was extracted with n-hexane (200 mL). The mixture was concentrated under vacuum. The residue was purified by column chromatography on silica gel using 70% petroleum ether and 30% ethyl acetate as eluent to afford 3-chloro-2,6-difluoro-5-nitropyridine (5.0 g, 74% yield) as an off-white solid. H NMR (300 MHz, Chloroform-d) δ 8.75 (s, 1H); LC-MS: m/z 195 [M+H]⁺.

### Step 2: tert-butyl 3-((5-chloro-6-fluoro-3-nitropyridin-2-yl)oxy)azetidine-1-carboxylate

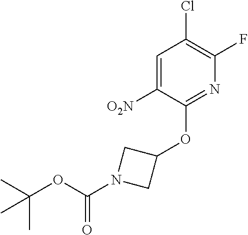 [SMILES: CC(C)(C)OC(=O)N1CC(Oc2nc(F)c(Cl)cc2[N+](=O)[O-])C1]

To a solution of tert-butyl 3-hydroxyazetidine-1-carboxylate (2.2 g, 12.7 mmol) in tetrahydrofuran (40 mL) was added NaH (728 mg, 19.0 mmol, 60% in mineral oil) at 0° C. in several portions. The mixture was stirred at 0° C. for 15 min. 3-chloro-2,6-difluoro-5-nitropyridine (3.1 g, 15.8 mmol) was added and the mixture was allowed to warm to 25° C. and stirred for 2 h. The reaction mixture was poured into crushed ice (200 g). The resulting solution was extracted with ethyl acetate (3×200 mL). The combined organic layers were dried over anhydrous sodium sulfate and concentrated under vacuum. The residue was purified by column chromatography on silica gel using 65% petroleum ether and 35% ethyl acetate as eluent to afford tert-butyl 3-((5-chloro-6-fluoro-3-nitropyridin-2-yl)oxy)azetidine-1-carboxylate (3.0 g, 54% yield) as an off-white solid. ¹H NMR (300 MHz, Chloroform-d) δ 8.58 (s, 1H), 5.40-5.46 (m, 1H), 4.34-4.48 (m, 2H), 4.04-4.15 (m, 2H), 1.48 (s, 9H). LC-MS: m/z 348 [M+H]⁺.

### Step 3: tert-butyl 3-((5-chloro-6-methoxy-3-nitropyridin-2-yl)oxy)azetidine-1-carboxylate

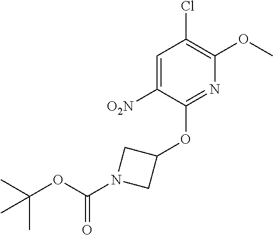 [SMILES: COc1nc(OC2CN(C(=O)OC(C)(C)C)C2)c([N+](=O)[O-])cc1Cl]

To a stirred solution of tert-butyl 3-((5-chloro-6-fluoro-3-nitropyridin-2-yl)oxy)azetidine-1-carboxylate (3.0 g, 8.6 mmol) in methanol (200 mL) was added sodium methanolate (483 mg, 8.6 mmol) in methanol (9 mL) at 25° C. by dropwise. The resulting mixture was stirred at 25° C. for 30 min under nitrogen atmosphere. The resulting mixture was concentrated under vacuum. The residue was purified by column chromatography on silica gel using 60% petroleum ether and 40% ethyl acetate as eluent to afford tert-butyl 3-((5-chloro-6-methoxy-3-nitropyridin-2-yl)oxy)azetidine-1-carboxylate (2.0 g, 64% yield) as a yellow solid. ¹H NMR (300 MHz, Chloroform-d) δ 8.48 (s, 1H), 5.48-5.35 (m, 1H), 4.39-4.45 (m, 2H), 4.22-4.08 (m, 2H), 4.08 (s, 3H), 1.48 (s, 9H). LC-MS: m/z 360 [M+H]⁺.

### Step 4: 2-(azetidin-3-yloxy)-5-chloro-6-methoxy-3-nitropyridine

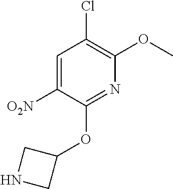 [SMILES: COc1nc(OC2CNC2)c([N+](=O)[O-])cc1Cl]

To a stirred solution of tert-butyl 3-((5-chloro-6-methoxy-3-nitropyridin-2-yl)oxy)azetidine-1-carboxylate (1.0 g, 2.7 mmol) in dichloromethane (25 mL) was added TFA (5 mL) at 25° C. The resulting mixture was stirred at 25° C. for 1 h and then concentrated under reduced pressure. The residue was purified by column chromatography on silica gel using 93% dichloromethane and 7% methanol as eluent to afford 2-(azetidin-3-yloxy)-5-chloro-6-methoxy-3-nitropyridine (700 mg, 66% yield) as a brown solid. LC-MS: m/z 260 [M+H]⁺.

### Step 5: 3-chloro-2-methoxy-6-((1-methylazetidin-3-yl)oxy)-5-nitropyridine

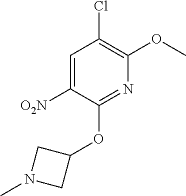

To a stirred solution of 2-(azetidin-3-yloxy)-5-chloro-6-methoxy-3-nitropyridine (700 mg, 2.7 mmol) in dichloromethane (25 mL) was added formaldehyde (323 mg, 3.2 mmol, 30% in water) and sodium triacetoxyborohydride (857 mg, 4.1 mmol) at 25° C. The resulting mixture was stirred at 25° C. for 2 h under nitrogen atmosphere. The reaction was quenched by the addition of water (100 mL), and the aqueous layer was extracted with dichloromethane (3×100 mL). The combined organic layers were dried over anhydrous sodium sulfate and concentrated under vacuum. The residue was purified by column chromatography using 50% petroleum ether and 50% ethyl acetate as eluent to give 3-chloro-2-methoxy-6-((1-methylazetidin-3-yl)oxy)-5-nitropyridine (500 mg, 68% yield) as an off white solid. LC-MS: m/z 274 [M+H]⁺.

### Step 6: 5-chloro-6-methoxy-2-((1-methylazetidin-3-yl)oxy)pyridin-3-amine

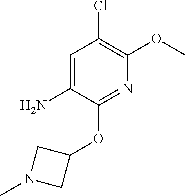

To a solution of 3-chloro-2-methoxy-6-((1-methylazetidin-3-yl)oxy)-5-nitropyridine (500 mg, 1.8 mmol) and NH₄Cl (293 mg, 5.4 mmol) in ethanol (40 mL) and water (12 mL) was added Fe (510 mg, 9.1 mmol). The reaction mixture was stirred at 80° C. for 2 h. The mixture was filtered through a pad of diatomaceous earth and the pad was washed with ethyl acetate (50 mL). The filtrate was concentrated under vacuum. The residue was diluted with water (80 mL) and extracted with ethyl acetate (3×80 mL). The combined organic extracts were washed with brine (300 mL), dried over anhydrous sodium sulfate and concentrated under vacuum. The residue was purified by column chromatography on silica gel using 90% dichloromethane and 10% methanol as eluent to afford 5-chloro-6-methoxy-2-((1-methylazetidin-3-yl)oxy)pyridin-3-amine (180 mg, 37% yield) as a brown solid. LC-MS: m/z 244 [M+H]⁺

### Step 7: (R)-2-chloro-N-(5-chloro-6-methoxy-2-((1-methylazetidin-3-yl)oxy)pyridin-3-yl)-8-methyl-8-(trifluoromethyl)-7,8-dihydro-6H-pyrazolo[1,5-a]pyrrolo[2,3-e]pyrimidine-6-carboxamide

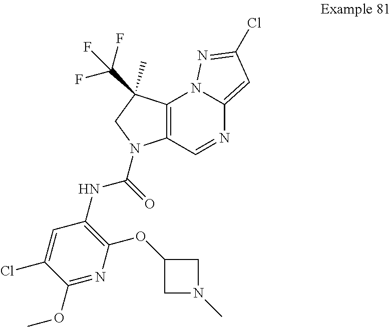 [SMILES: COc1nc(OC2CN(C)C2)c(NC(=O)N2C[C@@](C)(C(F)(F)F)c3c2cnc2cc(Cl)nn32)cc1Cl]

The title compound was prepared according to Method M1 Step 2 by using 5-chloro-6-methoxy-2-((1-methylazetidin-3-yl)oxy)pyridin-3-amine and Method M1 Isomer 2. The enantiomer of Example 81 can be prepared analogously using Method M1 isomer 1.

Example 81: ¹H NMR (300 MHz, Methanol-d₄) δ 9.30 (s, 1H), 8.01 (s, 1H), 6.79 (s, 1H), 5.30-5.38 (m, 1H), 4.73 (d, J=11.2 Hz, 1H), 4.16-4.22 (m, 3H), 3.99 (s, 3H), 3.69 (dd, J=10.8, 5.2 Hz, 2H), 2.67 (s, 3H), 2.05 (s, 3H). LC-MS: m/z 546 [M+H]⁺.

### Method E3

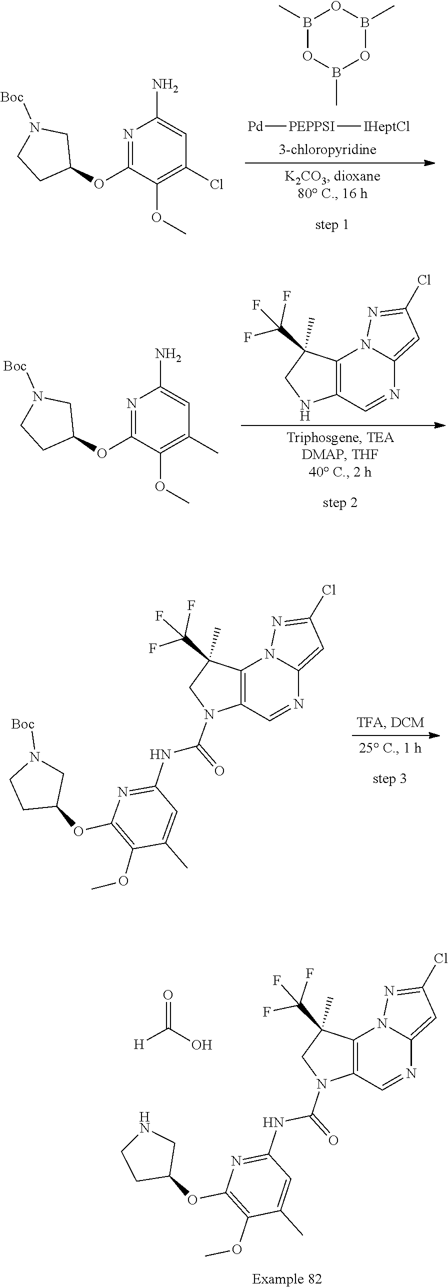

### Example 82: (R)-2-chloro-N-(5-methoxy-4-methyl-6-(((S)-pyrrolidin-3-yl)oxy)pyridin-2-yl)-8-methyl-8-(trifluoromethyl)-7,8-dihydro-6H-pyrazolo[1,5-a]pyrrolo[2,3-e]pyrimidine-6-carboxamide formate

### Step 1: tert-butyl (S)-3-((6-amino-3-methoxy-4-methylpyridin-2-yl)oxy)pyrrolidine-1-carboxylate

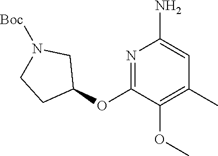 [SMILES: COc1c(C)cc(N)nc1O[C@H]1CCN(C(=O)OC(C)(C)C)C1]

To a stirred solution of tert-butyl (S)-3-((6-amino-4-chloro-3-methoxypyridin-2-yl)oxy)pyrrolidine-1-carboxylate (100 mg, 290.8 μmol; Method P2, step 7) in dioxane (10 mL) were added 2,4,6-trimethyl-1,3,5,2,4,6-trioxatriborinane (219 mg, 1.8 mmol), K₂CO₃ (80 mg, 581.7 μmol) and Pd-PEPPSI-IHeptCl 3-chloropyridine (28 mg, 29.1 μmol) in portions at 25° C. under nitrogen atmosphere. The mixture was stirred at 80° C. for 16 h. After cooled to 25° C., the resulting mixture was concentrated under vacuum. The residue was purified by Prep-TLC using 50% petroleum ether and 50% ethyl acetate as eluent to afford tert-butyl (S)-3-((6-amino-3-methoxy-4-methylpyridin-2-yl)oxy)pyrrolidine-1-carboxylate (50 mg, 16% yield) as a yellow oil. ¹H NMR (300 MHz, Methanol-d₄) δ 6.24 (s, 1H), 5.50-5.52 (m, 1H), 3.69 (s, 3H), 350-3.56 (m, 4H), 2.17-2.21 (m, 5H), 1.47 (s, 9H). LC-MS: m/z 324[M+H]⁺.

### Step 2: tert-butyl (S)-3-((6-((R)-2-chloro-8-methyl-8-(trifluoromethyl)-7,8-dihydro-6H-pyrazolo[1,5-a]pyrrolo[2,3-e]pyrimidine-6-carboxamido)-3-methoxy-4-methylpyridin-2-yl)oxy)pyrrolidine-1-carboxylate

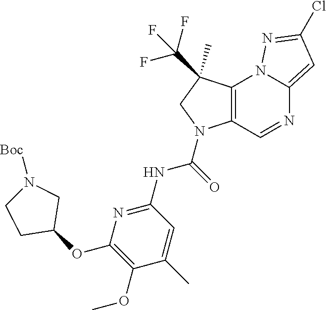 [SMILES: COc1c(C)cc(NC(=O)N2C[C@@](C)(C(F)(F)F)c3c2cnc2cc(Cl)nn32)nc1O[C@H]1CCN(C(=O)OC(C)(C)C)C1]

To a stirred mixture of tert-butyl (S)-3-((6-amino-3-methoxy-4-methylpyridin-2-yl)oxy)pyrrolidine-1-carboxylate (40 mg, 123.7 μmol) in tetrahydrofuran (2 mL) were added triphosgene (22 mg, 74.2 μmol) and TEA (18.7 mg, 185.5 μmol) at 25° C. The resulting mixture was stirred at 25° C. for 0.5 h and then filtered. The filtrate was added to a solution of Method M1 isomer 2 (34 mg, 123.7 μmol) in tetrahydrofuran (5 mL). To this solution were added N,N-dimethylpyridin-4-amine (30 mg, 247.4 μmol) and TEA (125 mg, 1.2 mmol). The mixture was stirred at 40° C. for 2 h. The mixture was concentrated under vacuum. The residue was purified by Prep-TLC using 97% dichloromethane and 3% methanol as eluent to afford tert-butyl (S)-3-((6-((R)-2-chloro-8-methyl-8-(trifluoromethyl)-7,8-dihydro-6H-pyrazolo[1,5-a]pyrrolo[2,3-e]pyrimidine-6-carboxamido)-3-methoxy-4-methylpyridin-2-yl)oxy)pyrrolidine-1-carboxylate (30 mg, 38% yield) as a white solid. LC-MS: m/z 626 [M+H]⁺

### Step 3: (R)-2-chloro-N-(5-methoxy-4-methyl-6-(((S)-pyrrolidin-3-yl)oxy)pyridin-2-yl)-8-methyl-8-(trifluoromethyl)-7,8-dihydro-6H-pyrazolo[1,5-a]pyrrolo[2,3-e]pyrimidine-6-carboxamide formate

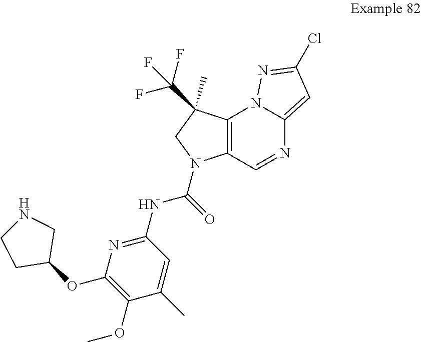 [SMILES: COc1c(C)cc(NC(=O)N2C[C@@](C)(C(F)(F)F)c3c2cnc2cc(Cl)nn32)nc1O[C@H]1CCNC1]

To a solution of tert-butyl (S)-3-((6-((R)-2-chloro-8-methyl-8-(trifluoromethyl)-7,8-dihydro-6H-pyrazolo[1,5-a]pyrrolo[2,3-e]pyrimidine-6-carboxamido)-3-methoxy-4-methylpyridin-2-yl)oxy)pyrrolidine-1-carboxylate (30 mg, 47.9 μmol) in dichloromethane (10 mL) was added TFA (1 mL) dropwise at 25° C. The reaction mixture was stirred at 25° C. for 1 h. The mixture was concentrated under vacuum. The crude product (30 mg) was purified by Prep-HPLC and the collected fractions were lyophilized to give Example 82 (8.6 mg, 29% yield) as a white solid. The epimer of Example 82 can be prepared analogously using Method M1 isomer 1.

Example 82: ¹H NMR (400 MHz, DMSO-d₆) δ 9.31 (s, 1H), 9.20-9.22 (m, 1H), 7.29 (s, 1H), 7.06 (d, J=1.2 Hz, 1H), 5.56-5.58 (m, 1H), 4.87-4.93 (m, 1H), 4.21-4.25 (m, 1H), 3.72 (s, 3H), 3.25-3.32 (m, 4H), 2.22 (s, 3H), 2.05-2.17 (m, 2H), 1.96 (s, 3H). LC-MS: m/z 526 [M+H]⁺.

### Method F3

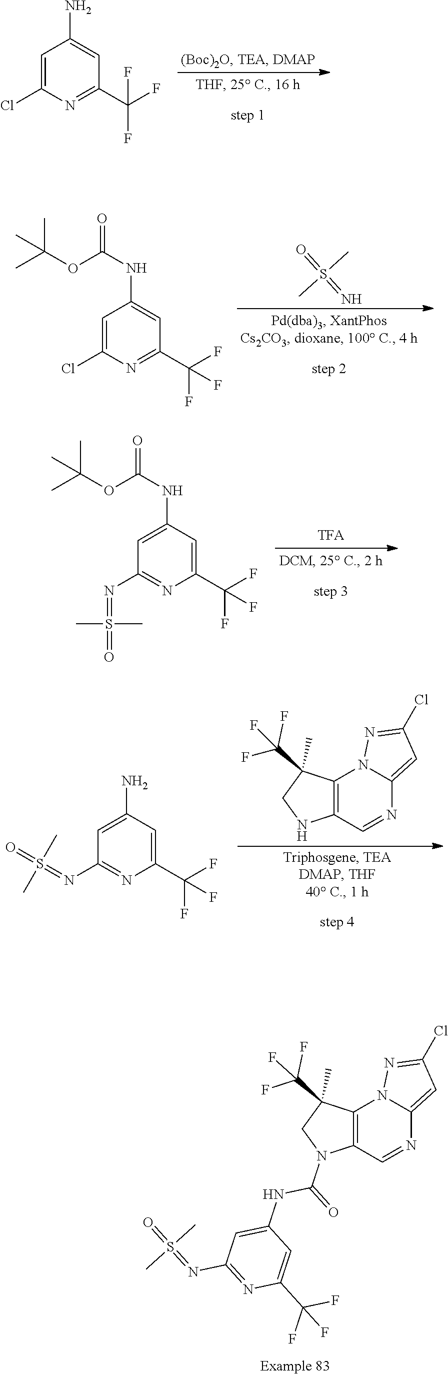

### Example 83: (R)-2-chloro-N-(2-((dimethyl(oxo)-λ⁶-sulfaneylidene)amino)-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

### Step 1: tert-butyl (2-chloro-6-(trifluoromethyl)pyridin-4-yl)carbamate

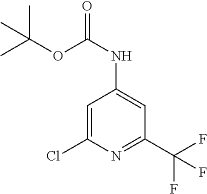 [SMILES: CC(C)(C)OC(=O)Nc1cc(Cl)nc(C(F)(F)F)c1]

To a stirred solution of 2-chloro-6-(trifluoromethyl)pyridin-4-amine (10 g, 50.9 mmol) in tetrahydrofuran (250 mL) were added di-tert-butyl dicarbonate (16.7 g, 76.3 mmol), TEA (12.9 g, 127.2 mmol) and N,N-dimethylpyridin-4-amine (0.6 g, 5.1 mmol). The resulting mixture was stirred at 25° C. for 16 h. The reaction mixture was concentrated under vacuum. The residue was purified by column chromatography on silica gel using 83% petroleum ether and 17% ethyl acetate as eluent to afford tert-butyl (2-chloro-6-(trifluoromethyl)pyridin-4-yl)carbamate (5.2 g, 33% yield) as a white solid. ¹H NMR (300 MHz, Chloroform-d) δ: 7.65 (s, 2H), 1.53 (s, 9H); LC-MS: m/z 297 [M+H]⁺

### Step 2: tert-butyl (2-((dimethyl(oxo)-λ⁶-sulfaneylidene)amino)-6-(trifluoromethyl)pyridin-4-yl)carbamate

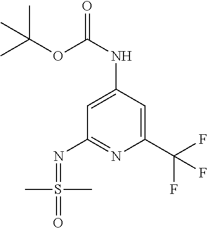 [SMILES: CC(C)(C)OC(=O)Nc1cc(N=S(C)(C)=O)nc(C(F)(F)F)c1]

To a stirred solution of tert-butyl (2-chloro-6-(trifluoromethyl)pyridin-4-yl)carbamate (600 mg, 2.0 mmol) in dioxane (12 mL) were added iminodimethyl-λ⁶-sulfanone (226 mg, 2.4 mmol), Pd₂(dba)₃ (186 mg, 0.2 mmol), XantPhos (235 mg, 0.4 mmol), Cs₂CO₃ (989 mg, 0.4 mmol) under nitrogen atmosphere. The resulting mixture was stirred at 100° C. for 3 h. The mixture was allowed to cool down to room temperature and was concentrated under reduced pressure. The residue was purified by column chromatography on silica gel using 50% petroleum ether and 50% ethyl acetate as eluent to afford tert-butyl (2-((dimethyl(oxo)-λ⁶-sulfaneylidene)amino)-6-(trifluoromethyl)pyridin-4-yl)carbamate (600 mg, 82% yield) as a yellow solid. ¹H NMR (300 MHz, Chloroform-d) δ: 9.96 (s, 1H), 7.45 (d, J=3 Hz, 1H), 6.88 (d, J=3 Hz, 1H), 3.38 (s, 6H), 1.49 (s, 9H); LC-MS: m/z 354 [M+H]⁺.

### Step 3: ((4-amino-6-(trifluoromethyl)pyridin-2-yl)imino)dimethyl-λ⁶-sulfanone

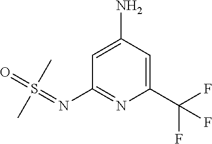 [SMILES: CS(C)(=O)=Nc1cc(N)cc(C(F)(F)F)n1]

To a stirred solution of tert-butyl (2-((dimethyl(oxo)-λ⁶-sulfaneylidene)amino)-6-(trifluoromethyl)pyridin-4-yl)carbamate (100 mg, 282.5 μmol) in dichloromethane (9.6 mL) was added TFA (2.4 mL). The mixture was stirred at 25° C. for 2 h. The resulting mixture was concentrated under vacuum. The residue was added saturated aqueous NaHCO₃ solution (40 mL) and extracted with ethyl acetate (3×40 mL). The combined organic layers were washed with brine (50 mL), dried over anhydrous Na₂SO₄ and concentrated under vacuum. The residue was purified by column chromatography on silica gel using 50% petroleum ether and 50% ethyl acetate as eluent to afford ((4-amino-6-(trifluoromethyl)pyridin-2-yl)imino)dimethyl-λ⁶-sulfanone (60 mg, 83% yield) as a white solid. LC-MS: m/z 254 [M+H]⁺.

### Step 4: (R)-2-chloro-N-(2-((dimethyl(oxo)-)⁶-sulfaneylidene)amino)-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

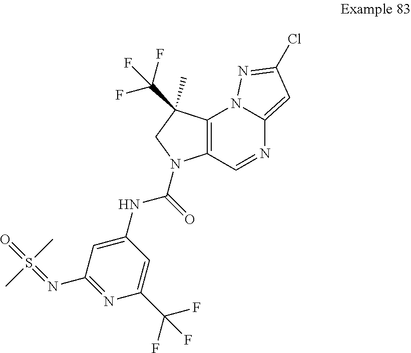 [SMILES: C[C@@]1(C(F)(F)F)CN(C(=O)Nc2cc(N=S(C)(C)=O)nc(C(F)(F)F)c2)c2cnc3cc(Cl)nn3c21]

To a stirred solution of ((4-amino-6-(trifluoromethyl)pyridin-2-yl)imino)dimethyl-λ⁶-sulfanone (42 mg, 166 μmol) in tetrahydrofuran (4 mL) were added triphosgene (20 mg, 65.2 μmol) and TEA (17 mg, 163 μmol). The resulting mixture was stirred at 25° C. for 0.5 h and then filtered. The filtrate was added to a solution of Method M1 isomer 2 (30 mg, 109 μmol) in THF (1 mL). To this solution was then added TEA (111 mg, 1.1 mmol) and N,N-dimethylpyridin-4-amine (27 mg, 218 μmol). The mixture was stirred at 40° C. for 12 h. The mixture was poured into water (40 mL) and extracted with ethyl acetate (3×40 mL). The combined organic layers were washed with brine (50 mL), dried over anhydrous Na₂SO₄ and concentrated under vacuum. The residue was purified by Prep-HPLC and the collected fractions were lyophilized to give (R)-2-chloro-N-(2-((dimethyl(oxo)-λ⁶-sulfaneylidene)amino)-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide (26 mg, 42% yield) as a white solid. The enantiomer of Example 83 can be prepared analogously using Method M1 isomer 1.

Example 83: ¹H NMR (400 MHz, DMSO-d₆) δ: 9.35 (s, 1H), 9.31 (s, 1H), 7.50 (d, J=1.6 Hz, 1H), 7.20 (d, J=1.6 Hz, 1H), 7.06 (s, 1H), 4.84 (d, J=12 Hz, 1H), 4.25 (d, J=12 Hz, 1H), 3.41 (s, 6H), 1.96 (s, 3H). LC-MS: m/z 556 [M+H]⁺.

### Method G3

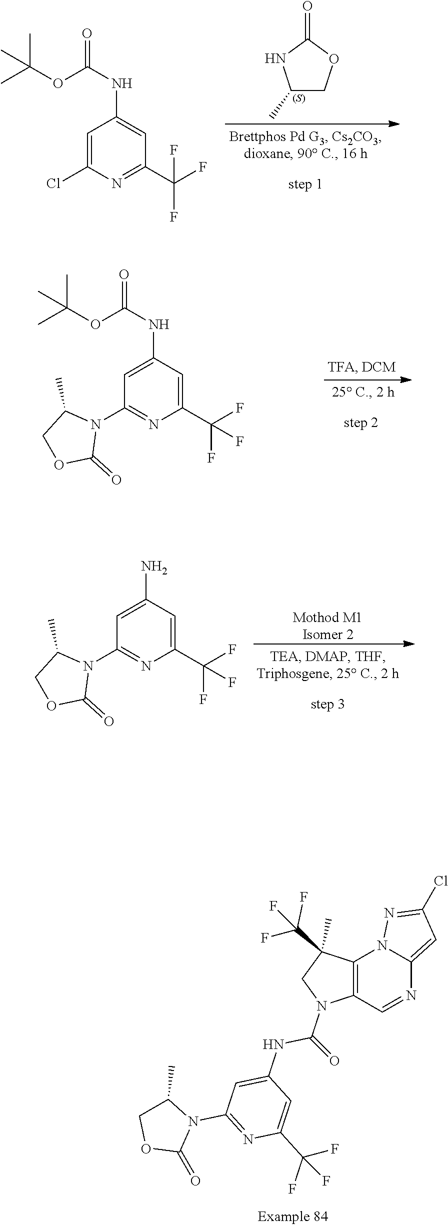

### Example 84: (R)-2-chloro-8-methyl-N-(2-((S)-4-methyl-2-oxooxazolidin-3-yl)-6-(trifluoromethyl)pyridine-4-yl)-8-(trifluoromethyl)-7,8-dihydro-6H-pyrazolo[1,5-a]pyrrolo[2,3-e]pyrimidine-6-carboxamide

### Step 1: tert-butyl (S)-(2-(4-methyl-2-oxooxazolidin-3-yl)-6-(trifluoromethyl)pyridin-4-yl)carbamate

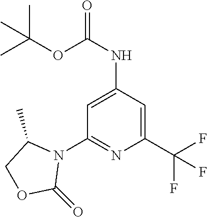 [SMILES: C[C@H]1COC(=O)N1c1cc(NC(=O)OC(C)(C)C)cc(C(F)(F)F)n1]

To a solution of tert-butyl (2-chloro-6-(trifluoromethyl)pyridin-4-yl)carbamate (200 mg, 675.6 μmol; Method F3, step 1), (S)-4-methyloxazolidin-2-one (69 mg, 675.6 μmol) in dioxane (10 mL) were added Cs₂CO₃ (439 mg, 1.3 mmol), Brettphos Pd G3 (13 mg, 135.1 μmol) under nitrogen atmosphere. The resulting mixture was stirred at 90° C. for 16 h. The reaction mixture was quenched with water (50 mL). The resulting solution was extracted with ethyl acetate (3×60 mL). The combined organic layers were dried over anhydrous sodium sulfate and concentrated under vacuum. The residue was purified by column chromatography on silica gel using 50% petroleum ether and 50% ethyl acetate as eluent to afford tert-butyl (S)-(2-(4-methyl-2-oxooxazolidin-3-yl)-6-(trifluoromethyl)pyridin-4-yl)carbamate (120 mg, 50% yield) as a yellow oil. ¹H NMR (300 MHz, DMSO-d₆) δ: 10.34 (s, 1H), 8.45 (d, J=3 Hz, 1H), 7.69 (d, J=3 Hz, 1H), 4.79-4.84 (m, 1H), 4.51-4.57 (m, 1H), 4.02-4.09 (m, 1H), 1.50 (s, 9H), 1.37 (d, J=6 Hz, 3H); LC-MS: m/z 362[M+H]⁺.

### Step 2: (S)-3-(4-amino-6-(trifluoromethyl)pyridin-2-yl)-4-methyloxazolidin-2-one

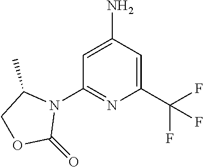 [SMILES: C[C@H]1COC(=O)N1c1cc(N)cc(C(F)(F)F)n1]

To a stirred solution of tert-butyl (S)-(2-(4-methyl-2-oxooxazolidin-3-yl)-6-(trifluoro methyl)pyridin-4-yl)carbamate (120 mg, 332.1 μmol) in dichloromethane (10 mL) was added TFA (2 mL) in portions at 25° C. The resulting mixture was stirred at 25° C. for 3 h. The resulting mixture was concentrated under reduced pressure. The residue was purified by column chromatography on silica gel using 93% dichloromethane and 7% methanol as eluent to afford (S)-3-(4-amino-6-(trifluoromethyl)pyridin-2-yl)-4-methyloxazolidin-2-one (80 mg, 60% yield) as a yellow oil. LC-MS: m/z 262 [M+H]⁺.

### Step 3: (R)-2-chloro-8-methyl-N-(2-((S)-4-methyl-2-oxooxazolidin-3-yl)-6-(trifluoromethyl)pyridin-4-yl)-8-(trifluoromethyl)-7,8-dihydro-6H-pyrazolo[1,5-a]pyrrolo[2,3-e]pyrimidine-6-carboxamide

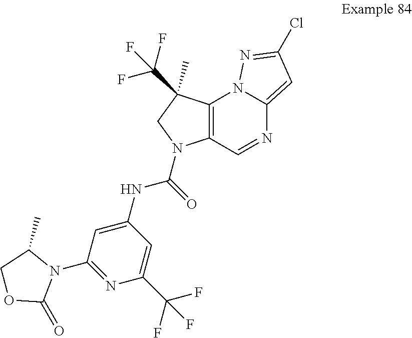 [SMILES: C[C@H]1COC(=O)N1c1cc(NC(=O)N2C[C@@](C)(C(F)(F)F)c3c2cnc2cc(Cl)nn32)cc(C(F)(F)F)n1]

The title compound was prepared according to Method M1 Step 2 by using (S)-3-(4-amino-6-(trifluoromethyl)pyridin-2-yl)-4-methyloxazolidin-2-one and Method M1 Isomer 2. The epimer of Example 84 can be prepared analogously using Method M1 isomer 1.

Example 84: ¹H NMR (400 MHz, DMSO-d₆) δ 9.89 (s, 1H), 9.36 (s, 1H), 8.54 (s, 1H), 8.01 (s, 1H), 7.08 (s, 1H), 4.90-4.79 (m, 2H), 4.56-4.59 (m, 1H), 4.31 (d, J=11.6 Hz, 1H), 4.11-4.14 (m, 1H), 1.97 (s, 3H), 1.40 (d, J=6.0 Hz, 3H). LC-MS: m/z 564 [M+H]⁺.

### Method H3

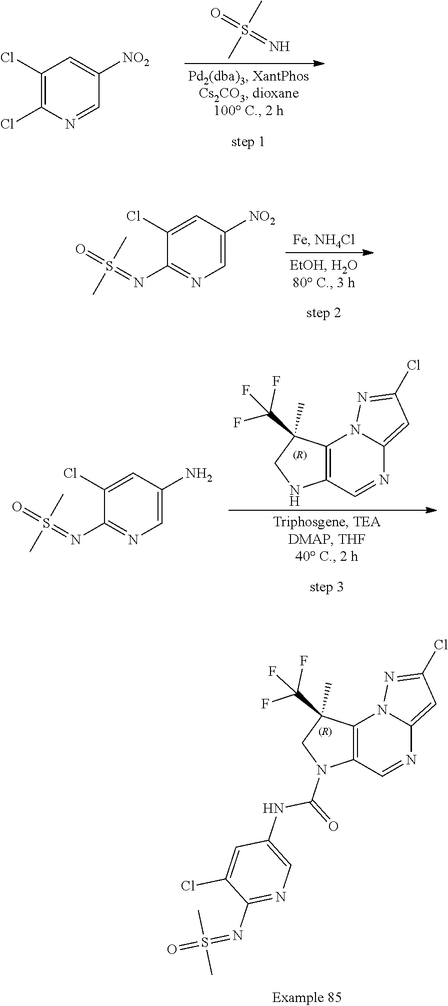

### Example 85: (R)-2-chloro-N-(5-chloro-6-((dimethyl(oxo)-λ⁶-sulfaneylidene)amino)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: ((3-chloro-5-nitropyridin-2-yl)imino)dimethyl-λ⁶-sulfanone

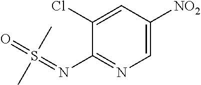

To a stirred solution of methyl 2,3-dichloro-5-nitropyridine (1.0 g, 5.2 mmol) and iminodimethyl-λ⁶-sulfanone (579.2 mg, 6.2 mmol) in dioxane (10 mL) were added Pd₂(dba)₃ (474.6 mg, 518.1 μmol), XantPhos (599.0 mg, 1.1 mmol) and Cs₂CO₃ (2.5 g, 7.7 mmol) under nitrogen atmosphere. The resulting mixture was stirred at 100° C. for 2 h. The mixture was cooled to 25° C. The reaction mixture was quenched by the addition of water (50 mL). The resulting solution was extracted with ethyl acetate (3×50 mL). The combined organic layers were dried over anhydrous sodium sulfate and concentrated under vacuum. The residue was purified by column chromatography on silica gel using 60% petroleum ether and 40% ethyl acetate as eluent to afford ((3-chloro-5-nitropyridin-2-yl)imino)dimethyl-λ⁶-sulfanone (700 mg, 48% yield) as a yellow solid. ¹H NMR (400 MHz, Chloroform-d) δ 8.96 (d, J=2.4 Hz, 1H), 8.38 (d, J=2.4 Hz, 1H), 3.47 (s, 6H). LC-MS: m/z 250 [M+H]⁺.

### Step 2: ((5-amino-3-chloropyridin-2-yl)imino)dimethyl-λ⁶-sulfanone

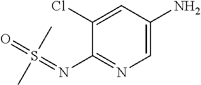

To a solution of ((3-chloro-5-nitropyridin-2-yl)imino)dimethyl-λ⁶-sulfanone (300 mg, 1.2 mmol) in ethanol (9 mL) and water (3 mL) were added Fe (335 mg, 6.0 mmol) and NH₄Cl (194 mg, 3.6 mmol). The resulting mixture was stirred at 80° C. for 3 h. The reaction mixture was quenched by the addition of water (50 mL). The resulting mixture was extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate as eluent to afford ((5-amino-3-chloropyridin-2-yl)imino)dimethyl-λ⁶-sulfanone (200 mg, 74% yield) as a yellow solid. ¹H NMR (400 MHz, Chloroform-d) δ 7.65 (d, J=2.4 Hz, 1H), 7.07 (d, J=2.4 Hz, 1H), 3.36 (s, 6H). LC-MS: m/z 220 [M+H]⁺.

### Step 3: (R)-2-chloro-N-(5-chloro-6-((dimethyl(oxo)-λ⁶-sulfaneylidene)amino)pyridin-3-yl)-8-methyl-8-(trifluoromethyl)-7,8-dihydro-6H-pyrazolo[1,5-a]pyrrolo[2,3-e]pyrimidine-6-carboxamide

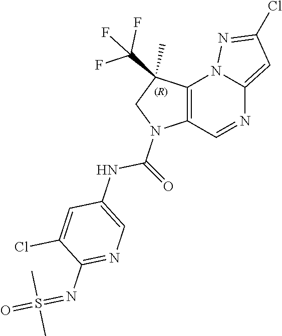

To a stirred solution of Method M1 isomer 2 (20 mg, 72.3 μmol) in tetrahydrofuran (2 mL) were added triphosgene (13 mg, 43.4 μmol) and TEA (11 mg, 108.4 μmol). The resulting mixture was stirred at 25° C. for 0.5 h and then filtered. The filtrate was added to a solution of ((5-amino-3-chloropyridin-2-yl)imino)dimethyl-λ⁶-sulfanone (16 mg, 72.3 μmol) in tetrahydrofuran (1 mL). To this solution were added N,N-dimethylpyridin-4-amine (18 mg, 144.6 μmol) and TEA (73 mg, 723.0 μmol). The mixture was stirred at 40° C. for 1 h. The mixture was concentrated under vacuum. The residue was purified by Prep-HPLC and the collected fractions were lyophilized to afford (R)-2-chloro-N-(5-chloro-6-((dimethyl(oxo)-λ⁶-sulfaneylidene)amino) pyridine-3-yl)-8-methyl-8-(trifluoromethyl)-7,8-dihydro-6H-pyrazolo[1,5-a]pyrrolo[2,3-e]pyrimidine-6-carboxamide (1.6 mg, 3% yield) as a white solid. The enantiomer of Example 85 can be prepared analogously using Method M1 isomer 1.

Example 85: ¹H NMR (400 MHz, Methanol-d₄) δ 9.34 (s, 1H), 8.18 (d, J=2.4 Hz, 1H), 7.94 (d, J=2.4 Hz, 1H), 6.77 (s, 1H), 4.72 (d, J=11.4 Hz, 1H), 4.16 (d, J=11.4 Hz, 1H), 3.43 (s, 6H), 2.03 (s, 3H). LC-MS: m/z 522 [M+H]⁺.

### Method 13

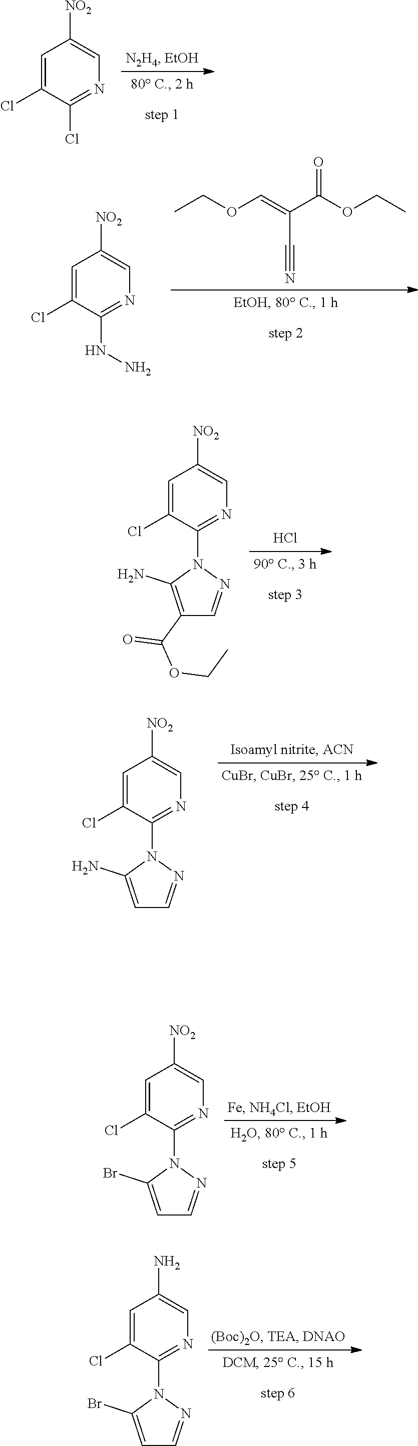

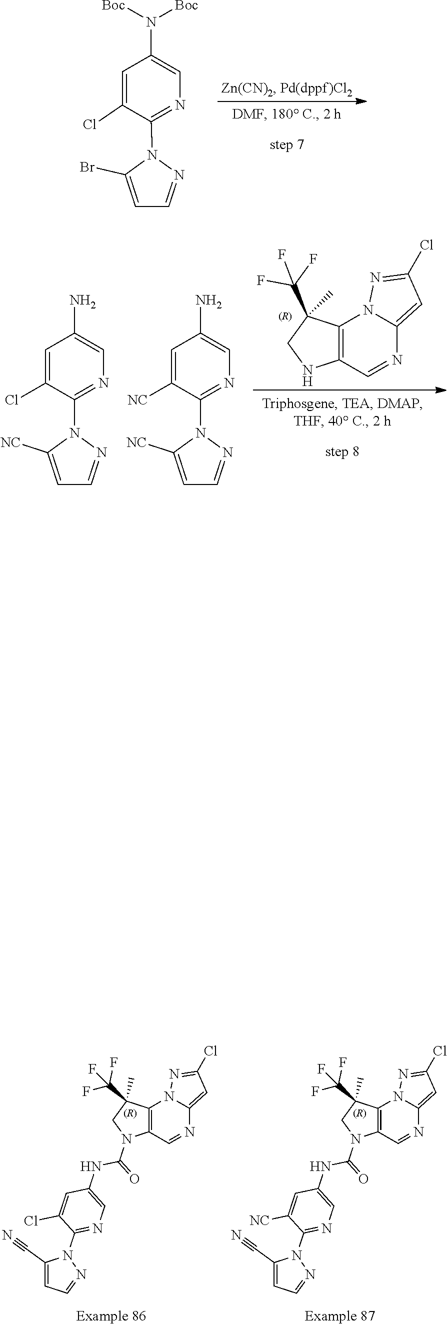

### Examples 86 and 87: (R)-2-chloro-N-(5-chloro-6-(5-cyano-1H-pyrazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(5-cyano-6-(5-cyano-1H-pyrazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 3-chloro-2-hydrazinyl-5-nitropyridine

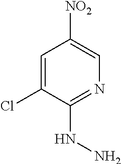 [SMILES: NNc1ncc([N+](=O)[O-])cc1Cl]

To a stirred mixture of 2,3-dichloro-5-nitropyridine (15.0 g, 77.7 mmol) in ethanol (200 mL) was added hydrazine hydrate (48.6 g, 777.2 mmol, 80%) at 25° C. The resulting mixture was stirred at 80° C. for 2 h. The precipitated product was collected by filtration and then re-crystallized from ethanol (200 mL) to afford 3-chloro-2-hydrazineyl-5-nitropyridine (12.0 g, 81% yield) as a light-yellow solid. LC-MS: m/z 189 [M+H]⁺.

### Step 2: ethyl 5-amino-1-(3-chloro-5-nitropyridin-2-yl)-1H-pyrazole-4-carboxylate

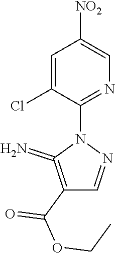

A solution of 3-chloro-2-hydrazinyl-5-nitropyridine (12.0 g, 63.6 mmol) and ethyl (E)-2-cyano-3-ethoxyacrylate (10.7 g, 63.6 mmol) in ethanol (80 mL) was stirred at 80° C. for 1 h. The mixture was cooled down to 25° C. and concentrated under vacuum. The residue was purified by column chromatography on silica gel using 20% petroleum ether and 80% ethyl acetate as eluent to afford ethyl 5-amino-1-(3-chloro-5-nitropyridin-2-yl)-1H-pyrazole-4-carboxylate (14 g, 70% yield) as a yellow solid. ¹H NMR (400 MHz, DMSO-d) 9.31 (d, J=2.4 Hz, 1H), 9.05 (d, J=2.4 Hz, 1H), 7.78 (s, 1H), 6.78 (br, 2H), 4.21 (d, J=7.2 Hz, 2H), 1.26 (d, J=7.2 Hz, 3H). LC-MS: m/z 312 [M+H]⁺.

### Step 3: 1-(3-chloro-5-nitropyridin-2-yl)-1H-pyrazol-5-amine

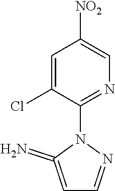

A solution of ethyl 5-amino-1-(3-chloro-5-nitropyridin-2-yl)-1H-pyrazole-4-carboxylate (3.1 g, 9.9 mmol) in concentrated hydrochloric acid (80 mL) was stirred at 90° C. for 3 h. The mixture was cooled to down to 25° C. The pH was adjusted to 8 with saturated aqueous NaHCO₃ solution. The mixture was extracted with ethyl acetate (3×55 mL). The combined organic layers were washed with brine (60 mL), dried over anhydrous sodium sulfate, and concentrated under vacuum. The residue was purified by column chromatography on silica gel using 50% petroleum ether and 50% ethyl acetate as eluent to afford 1-(3-chloro-5-nitropyridin-2-yl)-1H-pyrazol-5-amine (1.1 g, 46% yield) as a red solid. ¹H NMR (400 MHz, DMSO-d₆) δ 9.25 (d, J=3.2 Hz, 1H), 8.96 (d, J=3.6 Hz, 1H) 7.43 (d, J=2.4 Hz, 1H) 5.91 (br, 2H), 5.46 (d, J=2.4 Hz, 1H). LC-MS: m/z 240 [M+H]⁺.

### Step 4: 2-(5-bromo-1H-pyrazol-1-yl)-3-chloro-5-nitropyridine

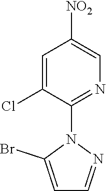 [SMILES: O=[N+]([O-])c1cnc(-n2nccc2Br)c(Cl)c1]

To a stirred mixture of 1-(3-chloro-5-nitropyridin-2-yl)-1H-pyrazol-5-amine (640 mg, 2.6 mmol) and isopentyl nitrite (312 mg, 2.6 mmol) in acetonitrile (10 mL) was added copper(II) bromide (596 mg, 2.6 mmol) and copper(I) bromide (383 mg, 2.6 mmol) at 0° C. under nitrogen atmosphere. The resulting mixture was stirred at 25° C. for 1 h. The resulting mixture was poured into water (30 mL) and extracted with ethyl acetate (3×35 mL). The combined organic layers were washed with brine (60 mL), dried over anhydrous sodium sulfate and concentrated under vacuum. The residue was purified by column chromatography on silica gel using 30% petroleum ether and 70% ethyl acetate as eluent to afford 2-(5-bromo-1H-pyrazol-1-yl)-3-chloro-5-nitropyridine (360 mg, 44% yield) as a yellow oil. LC-MS: m/z 303 [M+H]⁺.

### Step 5: 6-(5-bromo-1H-pyrazol-1-yl)-5-chloropyridin-3-amine

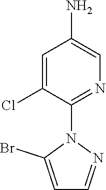 [SMILES: Nc1cnc(-n2nccc2Br)c(Cl)c1]

To a stirred solution of 2-(5-bromo-1H-pyrazol-1-yl)-3-chloro-5-nitropyridine (1.2 g, 3.9 mmol) in ethanol (24 mL) and water (8 mL) were added Fe (656 mg, 11.7 mmol) and NH₄Cl (1.0 g, 19.6 mmol). The mixture was stirred at 80° C. for 1 h. The mixture was allowed to cool down to 25° C. The mixture was diluted with water (50 mL). The resulting solution was extracted with ethyl acetate (3×50 mL). The organic layers were combined, dried over anhydrous sodium sulfate and concentrated under vacuum. The residue was purified by column chromatography on silica gel using 30% petroleum ether and 70% ethyl acetate as eluent to afford 6-(5-bromo-1H-pyrazol-1-yl)-5-chloropyridin-3-amine (820 mg, 72% yield) as a yellow solid. LC-MS: m/z 273 [M+H]⁺

### Step 6: tert-butyl N-[6-(5-bromo-1H-pyrazol-1-yl)-5-chloropyridin-3-yl]-N-[(tert-butoxy)carbonyl]carbamate

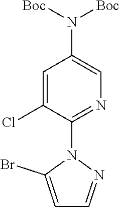 [SMILES: CC(C)(C)OC(=O)N(C(=O)OC(C)(C)C)c1cnc(-n2nccc2Br)c(Cl)c1]

To a stirred solution of 6-(5-bromopyrazol-1-yl)-5-chloro-pyridin-3-amine (820 mg, 3.0 mmol), TEA (910 mg, 8.9 mmol) and N,N-dimethylpyridin-4-amine (73 mg, 599.6 μmol) in dichloromethane (20 mL) was added tert-butoxycarbonyl tert-butyl carbonate (2.6 g, 11.9 mmol) at 0° C. The resulting mixture was stirred at 25° C. for 15 h. The resulting solution was concentrated under vacuum. The residue was purified by column chromatography on silica gel using 40% petroleum ether and 60% ethyl acetate as eluent to afford tert-butyl N-[6-(5-bromo-1H-pyrazol-1-yl)-5-chloropyridin-3-yl]-N-[(tert-butoxy)carbonyl]carbamate (980 mg, 68% yield) as a yellow solid. ¹H NMR (400 MHz, Chloroform-d) δ 8.35 (d, J=2.4 Hz, 1H), 7.77 (dd, J=8.4, 2.4 Hz, 2H), 6.52 (d, J=2.0 Hz, 1H), 1.46 (s, 18H). LC-MS: m/z 473 [M+H]⁺.

### Step 7: A mixture of 1-(5-amino-3-chloropyridin-2-yl)-1H-pyrazole-5-carbonitrile and 5-amino-2-(5-cyano-1H-pyrazol-1-yl)nicotinonitrile

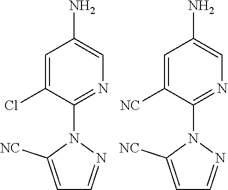

To a stirred solution of tert-butyl N-[6-(5-bromopyrazol-1-yl)-5-chloro-3-pyridyl]-N-tert-butoxycarbonyl-carbamate (800 mg, 1.7 mmol) in N,N-Dimethylformamide (12 mL) were added Zn(CN)₂ (396 mg, 3.4 mmol) and Pd(dppf)Cl₂ (276 mg, 337.7 μmol) under nitrogen atmosphere. The reaction mixture was heated in a microwave reactor at 180° C. for 2 h. The mixture was quenched by the addition of water (100 mL). The resulting mixture was extracted with ethyl acetate (3×100 mL). The combined organic layers were dried over anhydrous sodium sulfate and concentrated under vacuum. The residue was purified by column chromatography on silica gel using 60% petroleum ether and 40% ethyl acetate as eluent to afford a mixture of 1-(5-amino-3-chloropyridin-2-yl)-1H-pyrazole-5-carbonitrile and 5-amino-2-(5-cyano-1H-pyrazol-1-yl)nicotinonitrile (170 mg, 24% yield) as a yellow solid. LC-MS: m/z 220 and 211 [M+H]⁺.

### Step 8: (R)-2-chloro-N-(5-chloro-6-(5-cyano-1H-pyrazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(5-cyano-6-(5-cyano-1H-pyrazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide

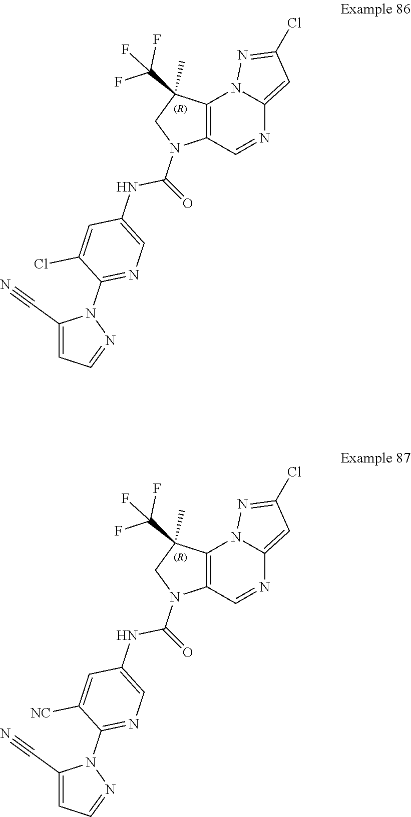

To a mixture of 2-(5-amino-3-chloro-2-pyridyl)pyrazole-3-carbonitrile and 5-amino-2-(5-cyanopyrazol-1-yl)pyridine-3-carbonitrile (170 mg, 819.6 μmol) in tetrahydrofuran (2 mL) were added TEA (62 mg, 614.7 μmol) and triphosgene (73 mg, 245.9 μmol). The mixture was stirred for 30 min at 25° C. and then filtered. The filtrate was added to a solution of Method M1 isomer 2 (91 mg, 327.8 μmol) in tetrahydrofuran (2 mL). To this solution was then added TEA (415 mg, 4.1 mmol) and N,N-dimethylpyridin-4-amine (100 mg, 819.6 μmol). The reaction mixture was stirred at 40° C. for 15 h. The solvent was removed under vacuum. The residue was purified by Prep-TLC using 50% petroleum ether and 50% ethyl acetate as eluent to afford 60 mg of crude product. The crude product was submitted to Prep-HPLC purification. The first eluting isomer was concentrated and lyophilized to afford (R)-2-chloro-N-(5-cyano-6-(5-cyano-1H-pyrazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide (Example 87; 5.6 mg, 0.5% yield) as a white solid. The second eluting isomer was concentrated and lyophilized to afford (R)-2-chloro-N-(5-chloro-6-(5-cyano-1H-pyrazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide (Example 86; 22.0 mg, 2.1% yield) as a white solid. The enantiomers of Example 86 and Example 87 can be prepared analogously using Method M1 isomer 1.

Example 86: ¹H NMR (300 MHz, DMSO-d₆) δ 9.75 (br, 1H), 9.36 (s, 1H), 8.75 (s, 1H), 8.53 (d, J=2.1 Hz, 1H), 8.10 (d, J=2.1 Hz, 1H), 7.52 (d, J=2.1 Hz, 1H), 7.07 (s, 1H), 4.85 (d, J=11.7 Hz, 1H), 4.30 (d, J=11.4 Hz, 1H), 1.99 (s, 3H). LC-MS: m/z 522 [M+H]⁺.

Example 87: ¹H NMR (300 MHz, DMSO-d₆) δ 9.76 (br, 1H), 9.36 (s, 1H), 8.94 (d, J=2.4 Hz, 1H), 8.70 (d, J=2.4 Hz, 1H), 8.18 (d, J=1.8 Hz, 1H), 7.61 (d, J=2.1 Hz, 1H), 7.08 (s, 1H), 4.83 (d, J=11.1 Hz, 1H), 4.30 (d, J=11.4 Hz, 1H), 1.99 (s, 3H). LC-MS: m/z 513 [M+H]⁺.

### Method J3

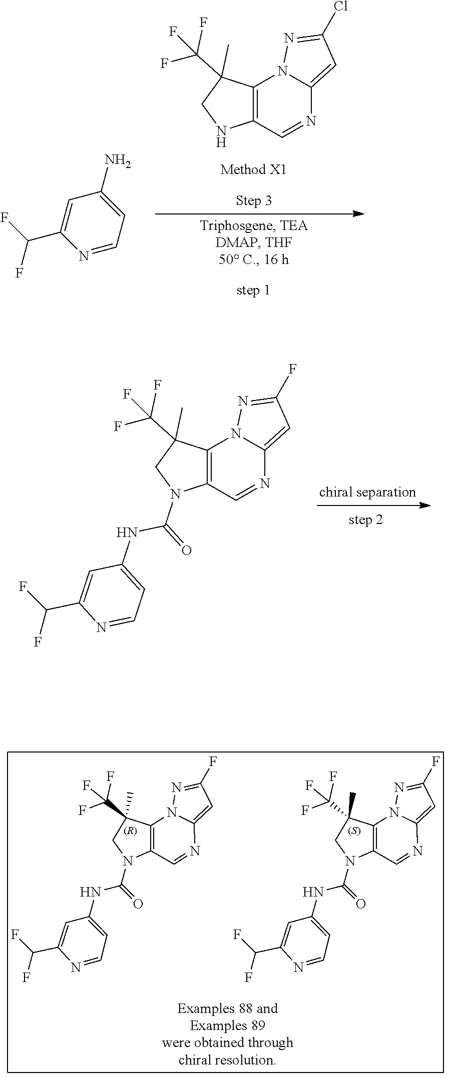

### Example 88 and 89: Single enantiomers obtained from a racemic mixture containing (R)—N-(2-(difluoromethyl)pyridin-4-yl)-2-fluoro-8-methyl-8-(trifluoromethyl)-7,8-dihydro-6H-pyrazolo[1,5-a]pyrrolo[2,3-e]pyrimidine-6-carboxamide and (S)—N-(2-(difluoromethyl)pyridin-4-yl)-2-fluoro-8-methyl-8-(trifluoromethyl)-7,8-dihydro-6H-pyrazolo[1,5-a]pyrrolo[2,3-e]pyrimidine-6-carboxamide

### Step 1: N-(2-(difluoromethyl)pyridin-4-yl)-2-fluoro-8-methyl-8-(trifluoromethyl)-7,8-dihydro-6H-pyrazolo[1,5-a]pyrrolo[2,3-e]pyrimidine-6-carboxamide

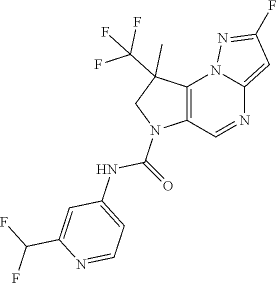 [SMILES: CC1(C(F)(F)F)CN(C(=O)Nc2ccnc(C(F)F)c2)c2cnc3cc(F)nn3c21]

To a stirred solution of 2-fluoro-8-methyl-8-(trifluoromethyl)-7,8-dihydro-6H-pyrazolo[1,5-a]pyrrolo[2,3-e]pyrimidine (Method X1, Step 3) (40 mg, 154 μmol) in THF (1 mL) were added triphosgene (30 mg, 102 μmol) and TEA (26 mg, 256 μmol) at 0° C. The resulting mixture was stirred at 25° C. for 1 h and then filtered. The filtrate was added to a solution of 2-(difluoromethyl)pyridin-4-amine (25 mg, 171 μmol) in THF (1 mL). To this solution was then added TEA (173 mg, 1.7 mmol) and N,N-dimethylpyridin-4-amine (31 mg, 256 μmol). The mixture was stirred at 50° C. for 16 h. The mixture was concentrated under vacuum. The residue was purified by Prep-HPLC and the collected fractions were lyophilized to give N-(2-(difluoromethyl)pyridin-4-yl)-2-fluoro-8-methyl-8-(trifluoromethyl)-7,8-dihydro-6H-pyrazolo[1,5-a]pyrrolo[2,3-e]pyrimidine-6-carboxamide (20 mg, 30% yield) as an off-white solid. LC-MS: m/z 431 [M+H]⁺.

### Step 2: Separation of enantiomers to obtain (R)—N-(2-(difluoromethyl)pyridin-4-yl)-2-fluoro-8-methyl-8-(trifluoromethyl)-7,8-dihydro-6H-pyrazolo[1,5-a]pyrrolo[2,3-e]pyrimidine-6-carboxamide and (S)—N-(2-(difluoromethyl)pyridin-4-yl)-2-fluoro-8-methyl-8-(trifluoromethyl)-7,8-dihydro-6H-pyrazolo[1,5-a]pyrrolo[2,3-e]pyrimidine-6-carboxamide

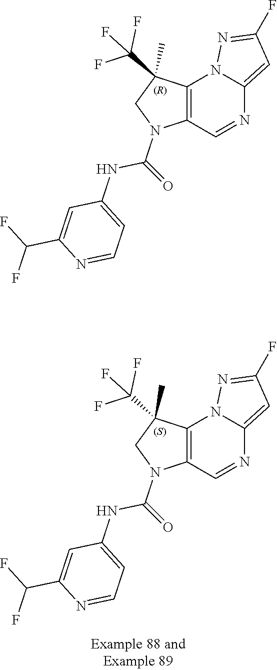

A sample containing 20 mg of N-(2-(difluoromethyl)pyridin-4-yl)-2-fluoro-8-methyl-8-(trifluoromethyl)-7,8-dihydro-6H-pyrazolo[1,5-a]pyrrolo[2,3-e]pyrimidine-6-carboxamide was submitted to chiral-HPLC (Column: Lux 5u Cellulose-4, 2.12*25 cm, 5 um; Mobile Phase A: Hex(0.5% 2M NH₃-MeOH)—HPLC, Mobile Phase B:EtOH-HPLC; Flow rate: 20 mL/min; Gradient: 10 B to 10 B in 21 min; 220/254 nm; RT1:12.058; RT2:17.004; Injection Volume: 1.5 ml; Number of Runs: 4). The first eluting isomer was concentrated and lyophilized to afford Example 88 (9.8 mg, 13% yield) as an off-white solid. The second eluting isomer was concentrated and lyophilized to afford Example 89 (6.5 mg, 9% yield) as an off-white solid.

Example 88: ¹H NMR (400 MHz, Chloroform-d) δ 9.38 (s, 1H), 8.53 (d, J=6.0 Hz, 1H), 8.10 (s, 1H), 8.03 (s, 1H), 6.76 (t, J=54.8 Hz, 1H), 6.36 (d, J=5.2 Hz, 1H), 4.77 (d, J=10.8 Hz, 1H), 4.18 (d, J=10.8 Hz, 1H), 2.05 (s, 3H). LC-MS: m/z 431 [M+H]⁺.

Example 89: ¹H NMR (400 MHz, Chloroform-d) δ 9.39 (s, 1H), 8.56 (d, J=5.6 Hz, 1H), 7.83 (s, 1H), 7.79 (d, J=5.6 Hz, 1H), 6.67 (t, J=55.2 Hz, 1H), 6.35 (d, J=5.2 Hz, 1H), 4.63 (d, J=10.4 Hz, 1H), 4.09 (d, J=10.4 Hz, 1H), 2.05 (s, 3H). LC-MS: m/z 431 [M+H]⁺.

### Method K3

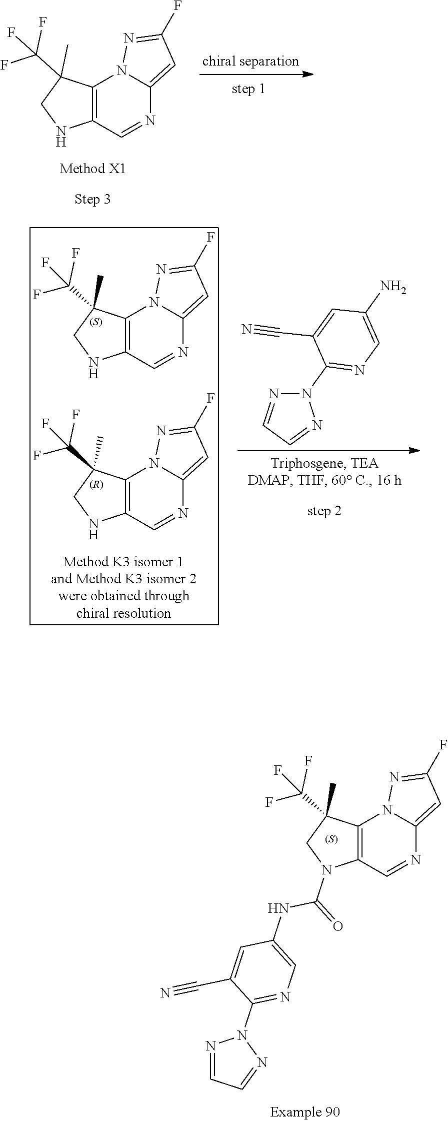

### Example 90: (S)—N-(5-cyano-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

### Step 1: Separation of enantiomers to obtain (S)-2-fluoro-8-methyl-8-(trifluoromethyl)-7,8-dihydro-6H-pyrazolo[1,5-a]pyrrolo[2,3-e]pyrimidine and (R)-2-fluoro-8-methyl-8-(trifluoromethyl)-7,8-dihydro-6H-pyrazolo[1,5-a]pyrrolo[2,3 e]pyrimidine

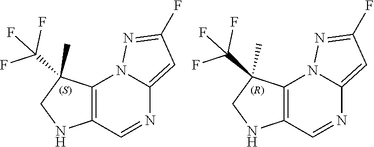

A racemic mixture of 2-fluoro-8-methyl-8-(trifluoromethyl)-7,8-dihydro-6H-pyrazolo[1,5-a]pyrrolo[2,3-e]pyrimidine (Method X1, Step 3; 500 mg) was submitted to chiral HPLC purification (Column: CHIRALPAK IG, 3*25 cm, 5 um; Mobile Phase A:CO₂, Mobile Phase B:MEOH(0.1% 2M NH₃-MEOH); Flow rate: 100 mL/min; Gradient: 20% B; 220 nm; RT1:2.13; RT2:3.52; Injection Volume: 4.8 ml; Number of Runs: 5). The first eluting isomer (RT 2.13 min) was concentrated and lyophilized to afford Method K3 isomer 1 (150 mg, 30% yield) as a yellow solid. The second eluting isomer (RT 3.52 min) was concentrated and lyophilized to afford Method K3 isomer 2 (100 mg, 20% yield) as a yellow solid. Both isomers were then individually subjected to Method X1, Step 4 for conversion to Example 38 and Example 39, respectively. Example 39 was derived from Method K3 isomer 2.

### Step 2: (S)—N-(5-cyano-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

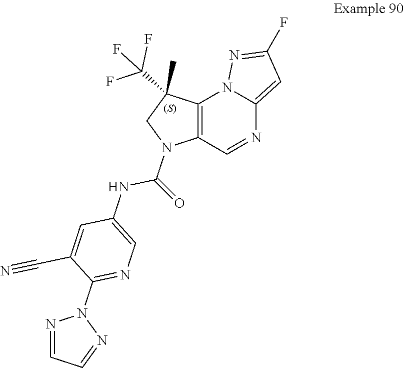

To a stirred solution of Method K3 Isomer 1 (30 mg, 115.3 μmol) in tetrahydrofuran (1 mL) were added triphosgene (20 mg, 69.2 μmol) and TEA (17 mg, 173.0 μmol). The resulting mixture was stirred at 25° C. for 0.5 h and then filtered. The filtrate was added to a solution of 5-amino-2-(2H-1,2,3-triazol-2-yl)nicotinonitrile (43 mg, 230.6 μmol) in tetrahydrofuran (1 mL). To this solution were added N,N-dimethylpyridin-4-amine (21 mg, 173.0 μmol) and TEA (117 mg, 1.2 mmol). The mixture was stirred at 60° C. for 16 h. The mixture was concentrated under vacuum. The residue was purified by Prep-HPLC and the collected fractions were lyophilized to afford (S)—N-(5-cyano-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide (3.6 mg, 6% yield) as a white solid.

Example 90: ¹H NMR (400 MHz, DMSO-d₆) δ 9.79 (s, 1H), 9.36 (s, 1H), 8.96 (s, 1H), 8.72 (d, J=2.4 Hz, 1H), 8.29 (s, 2H), 6.69 (d, J=4.8 Hz, 1H), 4.83 (d, J=11.6 Hz, 1H), 4.28 (d, J=11.6 Hz, 1H), 1.97 (s, 3H). LC-MS: m/z 473 [M+H]⁺.

### Example 91: (R)—N-(5-cyano-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

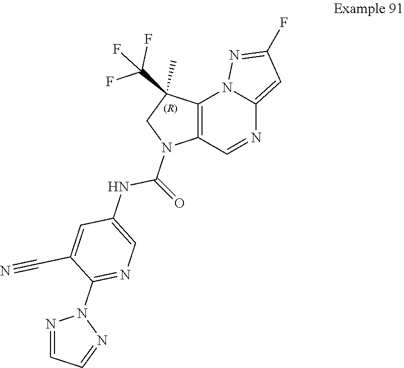

The title compound was prepared according to Method K3, Step 2 by using 5-amino-2-(2H-1,2,3-triazol-2-yl)nicotinonitrile and Method K3 Isomer 2.

Example 91: ¹H NMR (400 MHz, DMSO-d₆) δ: 9.76 (s, 1H), 9.35 (s, 1H), 8.98 (d, J=2.4 Hz, 1H), 8.72 (d, J=2.8 Hz, 1H), 8.30 (s, 2H), 6.70 (d, J=4.8 Hz, 1H), 4.84 (d, J=11.6 Hz, 1H), 4.30 (d, J=11.6 Hz, 1H), 1.98 (s, 3H). LC-MS: m/z 473 [M+H]⁺.

### Method L3

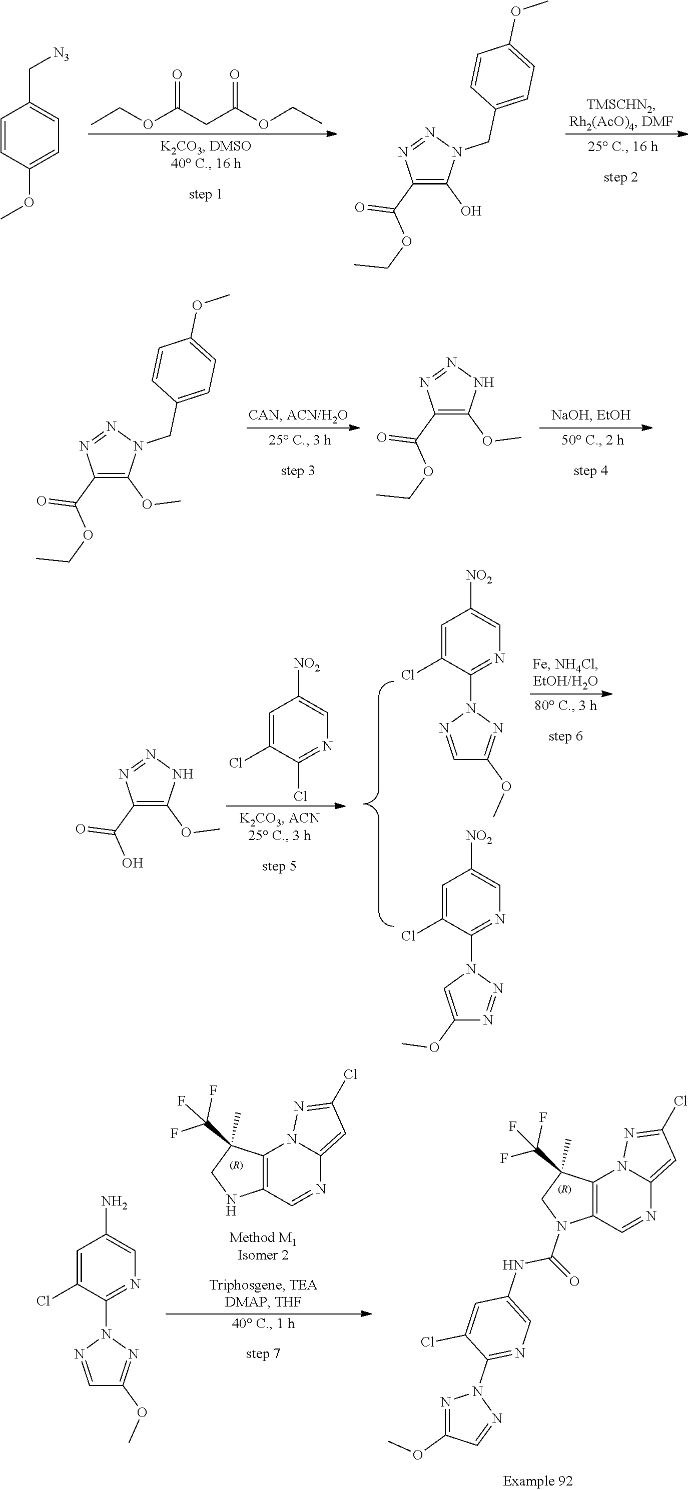

### Examples 92: (R)-2-chloro-N-(5-chloro-6(4-methoxy-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: ethyl 5-hydroxy-1-(4-methoxybenzyl)-1H-1,2,3-triazole-4-carboxylate

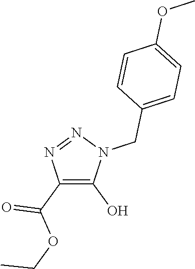 [SMILES: CCOC(=O)c1nnn(Cc2ccc(OC)cc2)c1O]

To a stirred mixture of 1-(azidomethyl)-4-methoxybenzene (4.0 g, 24.5 mmol) in dimethyl sulfoxide (30 mL) were added K₂CO₃ (13.5 g, 98.1 mmol) and diethyl malonate (5.5 g, 34.3 mmol) at 25° C. The reaction mixture was stirred at 40° C. for 16 h. The reaction mixture was cooled to 0° C. and quenched by addition of HCl (70 mL, 5 M). The mixture was stirred at 25° C. for 2 h. The solid was collected by filtration to give ethyl 5-hydroxy-1-(4-methoxybenzyl)-1H-1,2,3-triazole-4-carboxylate (4.7 g, 69% yield) as an off-white solid. ¹H NMR (300 MHz, Chloroform-d) δ 7.94 (s, 1H), 7.24-7.34 (m, 2H), 6.86-6.91 (m, 2H), 4.44-4.51 (m, 2H), 4.30 (q, J=7.2 Hz, 2H), 3.82 (s, 3H), 1.32 (t, J=7.2 Hz, 3H). LC-MS: m/z 278 [M+H]⁺.

### Step 2: ethyl 5-methoxy-1-(4-methoxybenzyl)-1H-1,2,3-triazole-4-carboxylate

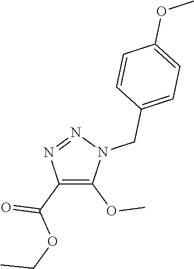 [SMILES: CCOC(=O)c1nnn(Cc2ccc(OC)cc2)c1OC]

To a stirred mixture of ethyl 5-hydroxy-1-(4-methoxybenzyl)-1H-1,2,3-triazole-4-carboxylate (6.0 g, 21.6 mmol) in N,N-Dimethylformamide (120 mL) were added Rh₂(AcO)₄ (68 mg, 246.7 μmol) and (trimethylsilyl)diazomethane solution (741 mg, 6.5 mmol). The reaction mixture stirred at 25° C. for 16 h. The mixture was cooled to 0° C. and quenched by addition of methanol (10 mL), glacial acetic acid (1 mL) and water (100 mL). The mixture was extracted with ethyl acetate (3×100 mL). The combine organic extracts were washed with brine (3×200 mL), dried over anhydrous sodium sulfate, filtered and concentrated. The residue was purified by column chromatography on silica gel using 50% petroleum ether and 50% ethyl acetate as eluent to afford ethyl 5-methoxy-1-(4-methoxybenzyl)-1H-1,2,3-triazole-4-carboxylate (3.4 g, 54% yield) as a colorless oil. ¹H NMR (300 MHz, Chloroform-d) δ 7.24-7.29 (m, 2H), 6.86-6.91 (m, 2H), 5.31 (s, 2H), 4.42 (q, J=7.2 Hz, 2H), 4.12 (s, 3H), 3.81 (s, 3H), 1.43 (t, J=7.2 Hz, 3H). LC-MS: m/z 292 [M+H]⁺.

### Step 3: ethyl 5-methoxy-1H-1,2,3-triazole-4-carboxylate

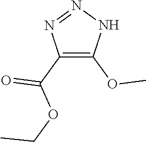

To a solution of ethyl 5-methoxy-1-(4-methoxybenzyl)-1H-1,2,3-triazole-4-carboxylate (3.0 g, 10.3 mmol) in acetonitrile (70 mL) and water (7 mL) was added CAN (5.6 g, 10.3 mmol). The resulting solution was stirred at 25° C. for 3 h. The reaction mixture was concentrated under reduced pressure. The residue was dissolved in water (100 mL) and extracted with ethyl acetate (2×100 mL). The combined organic layers were washed with brine (200 mL), dried over anhydrous sodium sulfate, and concentrated under vacuum. The residue was purified by column chromatography on silica gel using 50% petroleum ether and 50% ethyl acetate as eluent to afford ethyl 5-methoxy-1H-triazole-4-carboxylate (1.3 g, 74% yield) as a white solid. ¹H NMR (300 MHz, Chloroform-d) δ 12.69 (br, 1H), 4.49 (q, J=7.2 Hz, 2H), 4.12 (s, 3H), 1.43 (t, J=7.2 Hz, 3H). LC-MS: m/z 172 [M+H]⁺.

### Step 4: 5-methoxy-1H-1,2,3-triazole-4-carboxylic acid

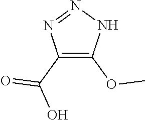

To a stirred solution of ethyl 5-methoxy-1H-triazole-4-carboxylate (560 mg, 3.3 mmol) in ethanol (10 mL) was added NaOH (785 mg, 19.2 mmol) in H₂O (3 mL). The resulting solution was stirred at 50° C. for 2 h. The pH was adjusted to 3 with HCl (6M). The mixture was extracted with ethyl acetate (3×50 mL). The combined organic layers were washed with brine (100 mL), dried with anhydrous sodium sulfate, and concentrated to afford 5-methoxy-1H-1,2,3-triazole-4-carboxylic acid (460 mg, 98% yield) as a colorless oil. ¹H NMR (300 MHz, Methanol-d₄) δ 4.02 (s, 3H). LC-MS: m/z 144 [M+H]⁺.

### Step 5: 3-chloro-2-(4-methoxy-2H-1,2,3-triazol-2-yl)-5-nitropyridine and 3-chloro-2-(4-methoxy-1H-1,2,3-triazol-2-yl)-5-nitropyridine

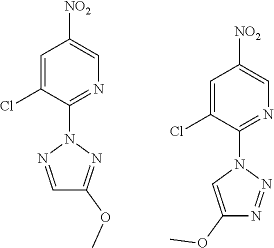 [SMILES: COc1cnn(-c2ncc([N+](=O)[O-])cc2Cl)n1 ; COc1cn(-c2ncc([N+](=O)[O-])cc2Cl)nn1]

A mixture of 5-methoxy-1H-1,2,3-triazole-4-carboxylic acid (400 mg, 2.8 mmol) in N,N-Dimethylformamide (8 mL) was stirred at 130° C. for 4 h. K₂CO₃ (2.4 g, 17.4 mmol) and 2,3-dichloro-5-nitro-pyridine (600 mg, 3.1 mmol) were added into the solution. The mixture was stirred at 25° C. for 3 h. The reaction mixture was quenched by water (100 mL), extracted with ethyl acetate (3×100 mL). The combined organic layers were washed with brine (100 mL), dried over anhydrous sodium sulfate and concentrated under vacuum. The residue was purified by column chromatography on silica gel using 70% petroleum ether and 30% ethyl acetate as eluent to afford 3-chloro-2-(4-methoxy-2H-1,2,3-triazol-2-yl)-5-nitropyridine (100 mg, 14% yield) as a white solid. ¹H NMR (300 MHz, Chloroform-d) δ 9.31 (d, J=2.4 Hz, 1H), 8.75 (d, J=2.4 Hz, 1H), 7.58 (s, 1H), 4.14 (s, 3H). LC-MS: m/z 256 [M+H]⁺ and 3-chloro-2-(4-methoxy-1H-1,2,3-triazol-2-yl)-5-nitropyridine (60 mg, 8% yield) as a white solid. ¹H NMR (300 MHz, Chloroform-d) δ 9.29 (d, J=2.4 Hz, 1H), 8.81 (d, J=2.4 Hz, 1H), 7.84 (s, 1H), 4.11 (s, 3H). LC-MS: m/z 256 [M+H]⁺.

### Step 6: 5-chloro-6-(4-methoxy-2H-1,2,3-triazol-2-yl)pyridin-3-amine

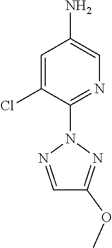

To a solution of 3-chloro-2-(4-methoxy-2H-1,2,3-triazol-2-yl)-5-nitropyridine (100 mg, 391.2 μmol) in ethanol (2 mL) and water (0.5 mL) were added Fe (65 mg, 1.3 mmol) and NH₄Cl (104 mg, 2.3 mmol). The resulting mixture was stirred at 80° C. for 3 h. The mixture was filtered. The filtrate was concentrated under vacuum, diluted with water (10 mL) and extracted with ethyl acetate (3×10 mL). The combined organic layers were washed with brine (2×20 mL), dried over anhydrous sodium sulfate and concentrated under vacuum. The residue was purified by column chromatography on silica gel using 40% petroleum ether and 60% ethyl acetate as eluent to afford 5-chloro-6-(4-methoxy-2H-1,2,3-triazol-2-yl) pyridin-3-amine (60 mg, 68% yield) as a white solid. ¹H NMR (300 MHz, Chloroform-d) δ 7.93 (d, J=2.7 Hz, 1H), 7.37 (s, 1H), 7.19 (d, J=2.7 Hz, 1H), 4.06 (s, 3H). LC-MS: m/z 226 [M+H]⁺.

### Step 7: (R)-2-chloro-N-(5-chloro-6-(4-methoxy-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

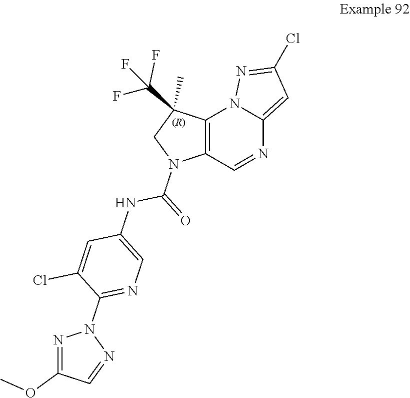

The title compound was prepared according to Method A2, Step 2 by using 5-chloro-6-(4-methoxytriazol-2-yl)pyridin-3-amine and Method M1 isomer 2. The enantiomer of Example 92 can be prepared analogously using Method M1 isomer 1.

Example 92: ¹H NMR (300 MHz, DMSO-d₆) δ 9.68 (s, 1H), 9.37 (s, 1H), 8.72 (d, J=2.4 Hz, 1H), 8.48 (d, J=2.4 Hz, 1H), 7.79 (s, 1H), 7.09 (s, 1H), 4.86 (d, J=11.4 Hz, 1H), 4.31 (d, J=11.4 Hz, 1H), 3.97 (s, 3H), 1.99 (s, 3H). LC-MS: m/z 528 [M+H]⁺.

### Method M3

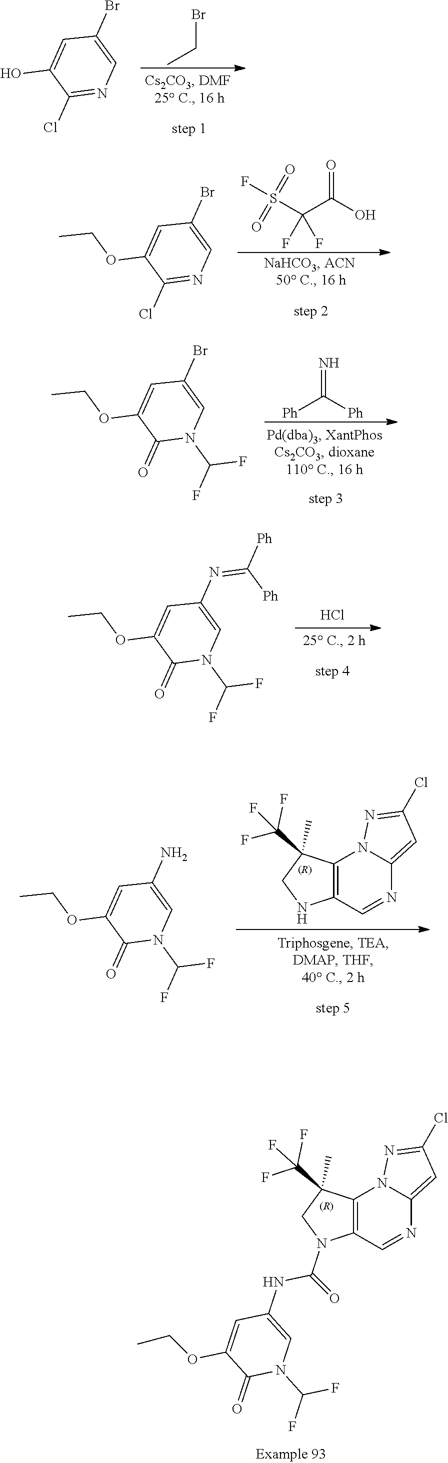

### Example 93: (R)-2-chloro-N-(1-(difluoromethyl-5-ethoxy-6-oxo-1,6-dihydropyridin-3-yl)-8-methyl-8-(trifluoromethyl)-7,8-dihydro-6H-pyrazolo[1,5-a]pyrrolo[2,3-e]pyrimidine-6-carboxamide

### Step 1: 5-bromo-2-chloro-3-ethoxypyridine

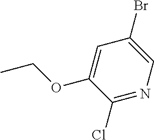

To a stirred mixture of 5-bromo-2-chloropyridin-3-ol (1.7 g, 8.2 mmol) and Cs₂CO₃ (4.0 g, 12.2 mmol) in N,N-dimethylformamide (20 mL) was added dropwise bromoethane (1.1 g, 9.8 mmol). The reaction mixture was stirred at 25° C. for 16 h. The reaction mixture was quenched with water (20 mL). The resulting solution was extracted with ethyl acetate (3×50 mL). The combined organic layers were washed with brine (50 mL), dried over anhydrous sodium sulfate and concentrated under vacuum. The residue was purified by column chromatography on silica gel using 70% petroleum ether and 30% ethyl acetate as eluent to afford 5-bromo-2-chloro-3-ethoxypyridine (1.7 g, 86% yield) as a white solid. ¹H NMR (400 MHz, Chloroform-d) δ 8.04 (d, J=2.0 Hz, 1H), 7.30 (d, J=2.0 Hz, 1H), 4.11 (q, J=7.2 Hz, 2H), 1.51 (t, J=7.2 Hz, 3H). LC-MS: m/z 236 [M+H]⁺.

### Step 2: 5-bromo-1-(difluoromethyl)-3-ethoxypyridin-2(1H)-one

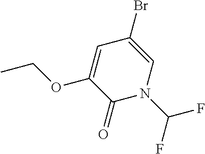

To a stirred solution of 5-bromo-2-chloro-3-ethoxypyridine (1.0 g, 4.1 mmol) in acetonitrile (10 mL) was added NaHCO₃ (418 mg, 5.0 mmol) and 2,2-difluoro-2-(fluorosulfonyl)acetic acid (2.2 g, 12.4 mmol). The reaction mixture was stirred at 50° C. for 16 h. The mixture was concentrated under vacuum. The residue was purified by column chromatography on silica gel using 70% petroleum ether and 30% ethyl acetate as eluent to afford 5-bromo-1-(difluoromethyl)-3-ethoxypyridin-2(1H)-one (690 mg, 61% yield) as a yellow oil. ¹H NMR (300 MHz, Chloroform-d) δ 7.69 (t, J=60.0 Hz, 1H), 7.21 (d, J=2.4 Hz, 1H), 6.61 (d, J=2.4 Hz, 1H), 4.02 (q, J=6.9 Hz, 2H), 1.49 (t, J=7.2 Hz, 3H). LC-MS: m/z 268 [M+H]⁺.

### Step 3: 1-(difluoromethyl)-5-((diphenylmethylene)amino)-3-ethoxypyridin-2(1H)-one

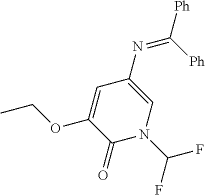 [SMILES: CCOc1cc(N=C(c2ccccc2)c2ccccc2)cn(C(F)F)c1=O]

To a mixture of 5-bromo-1-(difluoromethyl)-3-ethoxypyridin-2(1H)-one (500 mg, 1.8 mmol) in dioxane (20 mL) were added diphenylmethanimine (662 mg, 3.7 mmol), Pd₂(dba)₃ (568 mg, 548 μmol), Xantphos (317 mg, 548 μmol) and Cs₂CO₃ (1.8 g, 5.5 mmol) under nitrogen atmosphere. The resulting mixture was stirred at 110° C. for 16 h. The solid was filtered out. The filtrate was concentrated under vacuum. The residue was purified by column chromatography on silica gel using 90% petroleum ether and 10% ethyl acetate as eluent to afford 1-(difluoromethyl)-5-((diphenylmethylene)amino)-3-ethoxypyridin-2(1H)-one (330 mg, 49% yield) as a red solid. LC-MS: m/z 369 [M+H]⁺.

### Step 4: 5-amino-1-(difluoromethyl)-3-ethoxypyridin-2(1H)-one

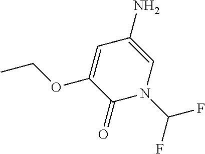

To a solution of 1-(difluoromethyl)-5-((diphenylmethylene)amino)-3-ethoxypyridin-2(1H)-one (330 mg, 895.8 μmol) in tetrahydrofuran (10 mL) was added hydrochloride (5 mL, 1 M). The resulting mixture was stirred at 25° C. for 2 h. The mixture was concentrated under vacuum. The residue was diluted with water (5 mL). The pH was adjusted to 7-8 with saturated aqueous NaHCO₃ solution. The resulting solution was extracted with ethyl acetate (3×5 mL). The organic layers were combined, dried over anhydrous sodium sulfate and concentrated under vacuum. The residue was purified by column chromatography on silica gel using 50% petroleum ether and 50% ethyl acetate as eluent to afford 5-amino-1-(difluoromethyl)-3-ethoxypyridin-2(1H)-one (180 mg, 81% yield) as a red solid. ¹H NMR (300 MHz, Chloroform-d) δ 7.74 (t, J=60.0 Hz, 1H), 6.54 (d, J=2.4 Hz, 1H), 6.32 (d, J=2.4 Hz, 1H), 4.00 (q, J=7.2 Hz, 2H), 1.48 (t, J=7.2 Hz, 3H). LC-MS: m/z 205 [M+H]⁺.

### Step 6: (R)-2-chloro-N-(1-(difluoromethyl)-5-ethoxy-6-oxo-1,6-dihydropyridin-3-yl)-8-methyl-8-(trifluoromethyl)-7,8-dihydro-6H-pyrazolo[1,5-a]pyrrolo[2,3-e]pyrimidine-6-carboxamide

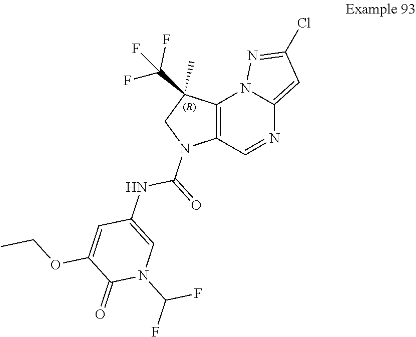

To a stirred solution of Method M1 isomer 2 (56 mg, 204.1 μmol) in tetrahydrofuran (5 mL) were added triphosgene (30 mg, 102.0 μmol) and TEA (31 mg, 306.1 μmol). The resulting mixture was stirred at 25° C. for 0.5 h and then filtered. The filtrate was added to a solution of 5-amino-1-(difluoromethyl)-3-ethoxypyridin-2(1H)-one (50 mg, 244.9 μmol) in tetrahydrofuran (5 mL). To this solution were added N,N-dimethylpyridin-4-amine (50 mg, 408.1 μmol) and TEA (206 mg, 2.0 mmol). The mixture was stirred at 40° C. for 2 h. The mixture was concentrated under vacuum. The residue was purified by Prep-HPLC and the collected fractions were lyophilized to afford (R)-2-chloro-N-(1-(difluoromethyl)-5-ethoxy-6-oxo-1,6-dihydropyridin-3-yl)-8-methyl-8-(trifluoromethyl)-7,8-dihydro-6H-pyrazolo[1,5-a]pyrrolo[2,3-e]pyrimidine-6-carboxamide (5.0 mg, 5% yield) as a yellow solid. The enantiomer of Example 93 can be prepared analogously using Method M1 isomer 1.

Example 93: ¹H NMR (300 MHz, DMSO-d₆) S 9.30 (s, 1H), 8.84 (s, 1H), 7.94 (t, J=60.0 Hz, 1H), 7.68 (d, J=2.4 Hz, 1H), 7.05 (d, J=2.4 Hz, 1H), 7.03 (s, 1H), 4.70 (d, J=11.4 Hz, 1H), 4.18 (d, J=11.7 Hz, 1H), 3.99 (q, J=6.9 Hz, 2H), 1.95 (s, 3H), 1.36 (t, J=6.9 Hz, 3H). LC-MS: m/z 507 [M+H]⁺.

### Method N3

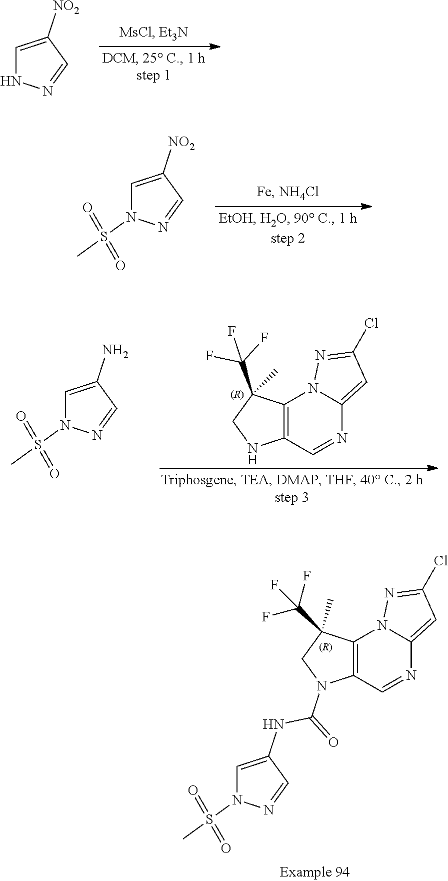

### Example 94: (R)-2-chlor-8-methyl-N-(1-(methylsulfonyl)-1H-pyrazol-4-yl)-8-(trifluoromethyl)-7,8-dihydro-6H-pyrazolo[1,5-a]pyrrolo[2,3-e]pyrimidine-6-carboxamide

### Step 1: 1-(methylsulfonyl)-4-nitro-1H-pyrazole

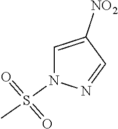

To a stirred solution of 4-nitro-1H-pyrazole (1.0 g, 8.8 mmol) in dichloromethane (20 mL) were added TEA (1.3 g, 13.3 mmol) and methanesulfonyl chloride (1.2 g, 10.6 mmol) at 25° C. The mixture was stirred at 25° C. for 1 h. The mixture was quenched by the addition of water (100 mL). The resulting mixture was extracted with dichloromethane (3×100 mL). The combined organic layers were dried over anhydrous sodium sulfate and concentrated under vacuum. The residue was purified by column chromatography on silica gel using 70% petroleum ether and 30% ethyl acetate as eluent to afford 1-(methylsulfonyl)-4-nitro-1H-pyrazole (1.4 g, 57% yield) as a white solid. ¹H NMR (300 MHz, DMSO-d) δ 9.37 (s, 1H), 8.71 (s, 1H), 3.76 (s, 3H). LC-MS: m/z 192 [M+H]⁺.

### Step 2: 1-(methylsulfonyl)-1H-pyrazol-4-amine

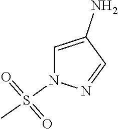

To a solution of 1-(methylsulfonyl)-4-nitro-1H-pyrazole (300 mg, 1.6 mmol) in ethanol (9 mL) and water (3 mL) were added NH₄Cl (420 mg, 7.9 mmol) and Fe (263 mg, 4.7 mmol). The resulting mixture was stirred at 90° C. for 1 h. After cooled to 25° C., the mixture was filtered. The filtrate was poured into water (50 mL) and extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate as eluent to afford 1-(methylsulfonyl)-1H-pyrazol-4-amine (190 mg, 47% yield) as a yellow solid. ¹H NMR (300 MHz, DMSO-d₆) δ 7.52 (s, 1H), 7.32 (s, 1H), 4.49 (s, 2H), 3.31 (m, 3H) LC-MS: m/z 162 [M+H]⁺.

### Step 3: (R)-2-chloro-8-methyl-N-(1-(methylsulfonyl)-1H-pyrazol-4-yl)-8-(trifluoromethyl)-7,8-dihydro-6H-pyrazolo[1,5-a]pyrrolo[2,3-e]pyrimidine-6-carboxamide

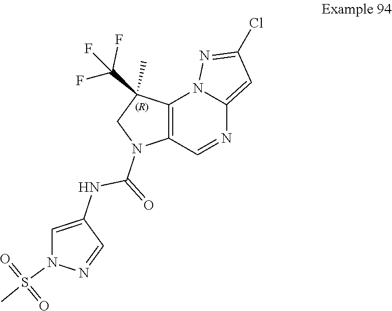

The title compound was prepared according to Method O1, Step 2 by using 1-(methylsulfonyl)-1H-pyrazol-4-amine and Method M1 isomer 2. The enantiomer of Example 94 can be prepared analogously using Method M1 isomer 1.

Example 94: ¹H NMR (300 MHz, DMSO-d₆) δ 9.58 (br, 1H), 9.36 (s, 1H), 8.28 (s, 1H), 8.02 (s, 1H), 7.06 (s, 1H), 4.69 (d, J=11.4 Hz, 1H), 4.21 (d, J=11.1 Hz, 1H), 3.52 (s, 3H), 1.97 (s, 3H). LC-MS: m/z 464 [M+H]⁺.

### Example 95: (R)-2-chloro-N-(5-chloro-6-(4-methoxy-1H-1,2,3-triazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide

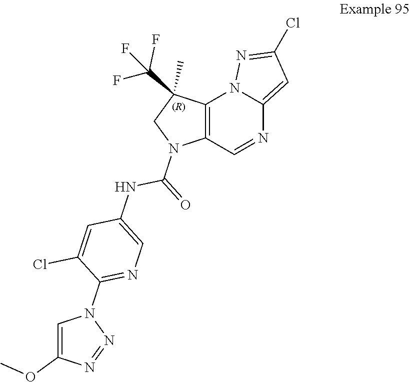

The title compound was prepared according to Method L3, Steps 6 and 7 by using 3-chloro-2-(4-methoxy-1H-1,2,3-triazol-2-yl)-5-nitropyridine and Method M1 isomer 2. The enantiomer of Example 95 can be prepared analogously using Method M1 isomer 1.

Example 95: ¹H NMR (300 MHz, DMSO-d₆) δ 9.72 (s, 1H), 9.36 (s, 1H), 8.76 (d, J=2.4 Hz, 1H), 8.51 (d, J=2.4 Hz, 1H), 8.20 (s, 1H), 7.09 (s, 1H), 4.86 (d, J=11.7 Hz, 1H), 4.31 (d, J=11.7 Hz, 1H), 3.93 (s, 3H), 2.00 (s, 3H). LC-MS: m/z 528 [M+H]⁺.

### Method O3

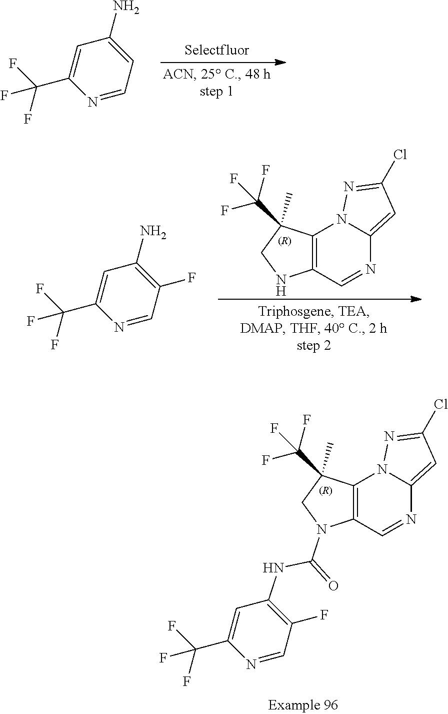

### Examples 96: (R)-2-chloro-N-(5-fluoro-2-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

### Step 1: 5-fluoro-2-(trifluoromethyl)pyridin-4-amine

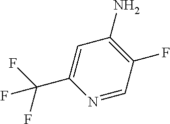 [SMILES: Nc1cc(C(F)(F)F)ncc1F]

To a stirred solution of 2-(trifluoromethyl)pyridin-4-amine (3 g, 18.5 mmol) in acetonitrile (50 mL) was added 1-(chloromethyl)-4-fluoro-1,4-diazabicyclo[2.2.2]octane-1,4-diium tetrafluoroborate (14 g, 40.7 mmol). The mixture was stirred at 25° C. for 48 h. The solvent was removed under vacuum. To the residue were added ethyl acetate (50 mL) and saturated aqueous NaHCO₃ solution (50 mL). The layers were separated, and the aqueous layer was extracted with ethyl acetate (2×50 mL). The combined organic layers were concentrated under vacuum. The residue was purified by column chromatography on silica gel using 97% dichloromethane and 3% methanol as eluent to afford 5-fluoro-2-(trifluoromethyl)pyridin-4-amine (310 mg, 8% yield) as a yellow solid. ¹H NMR (300 MHz, Chloroform-d) δ: 8.26 (d, J=2.7 Hz, 1H), 7.06 (d, J=6.8 Hz, 1H), 4.66 (s, 2H). LC-MS: m/z 181 [M+H]⁺.

### Step 2: (R)-2-chloro-N-(5-fluoro-2-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

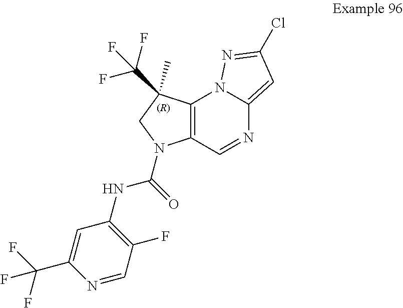

The title compound was prepared according to Method O1, Step 3 by using 5-fluoro-2-(trifluoromethyl)pyridin-4-amine and Method M1 isomer 2. The enantiomer of Example 96 can be prepared analogously using Method M1 isomer 1.

Example 96: ¹H NMR (400 MHz, DMSO-d₆) δ: 9.70 (s, 1H), 9.27 (s, 1H), 8.76 (d, J=2.6 Hz, 1H), 8.36 (d, J=6.0 Hz, 1H), 7.06 (s, 1H), 4.96 (d, J=11.6 Hz, 1H), 4.29 (d, J=11.5 Hz, 1H), 1.94 (s, 3H). LC-MS: m/z 483 [M+H]⁺.

### Method P3

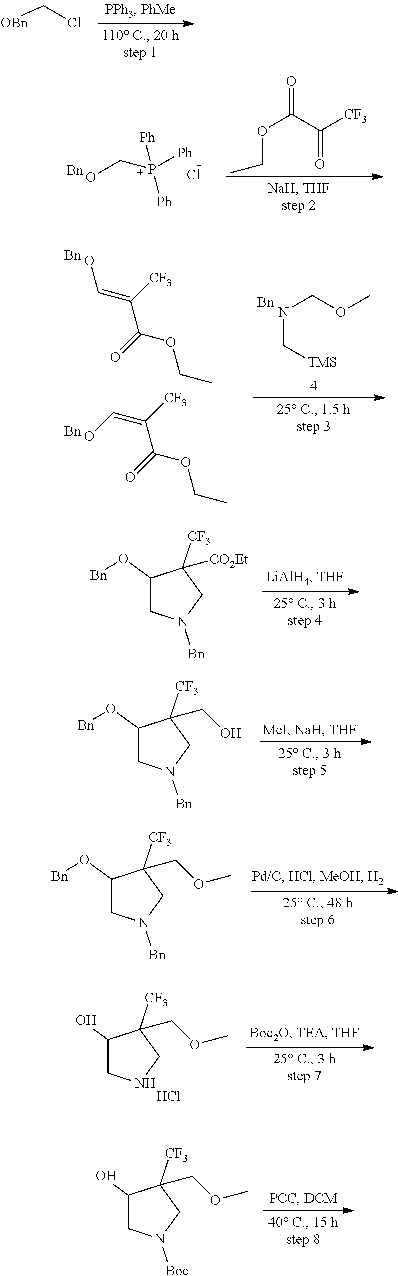

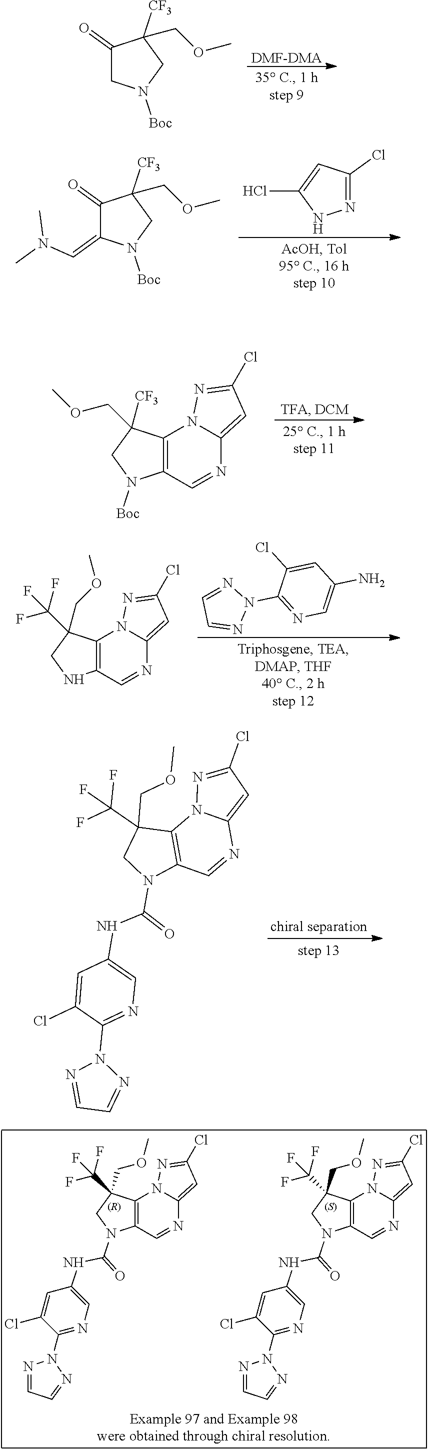

### Example 97 an 98: Single enantiomers obtained from a racemic mixture containing (R)-2-chloro-N-(5-chloro-6-(2H-1,2,3-triazol-2-yl)pyridin-3-yl)-8-(methoxymethyl)-8-(trifluoromethyl)-7,8-dihydro-6H-pyrazolo[1,5-a]pyrrolo[2,3-e]pyrimidine-6-carboxamide and (S)-2-chloro-N-(5-chloro-6-(2H-1,2,3-triazol-2-yl)pyridin-3-yl)-8-(methoxymethyl)-8-(trifluoromethyl)-7,8-dihydro-6H-pyrazolo[1,5-a]pyrrolo[2,3-e]pyrimidine-6-carboxamide

### Step 1: ((benzyloxy)methyl)triphenylphosphonium chloride

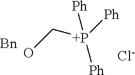 [SMILES: OC[P+](c1ccccc1)(c1ccccc1)c1ccccc1 ; [Cl-]]

To a stirred solution of ((chloromethoxy)methyl)benzene (50 g, 319.3 mmol) in toluene (100 mL) was slowly added triphenylphosphane (92.1 g, 351.2 mmol). The resulting mixture was stirred at 110° C. for 20 h. The mixture was filtrated, and the solid was washed with 50 mL petroleum ether to afford ((benzyloxy)methyl)triphenylphosphonium chloride (90 g, 66% yield) as a white solid. LC-MS: m/z 383 [M-Cl]⁺.

### Step 2: ethyl (Z)-3-(benzyloxy)-2-(trifluoromethyl)acrylate and ethyl (E)-3-(benzyloxy)-2-(trifluoromethyl)acrylate

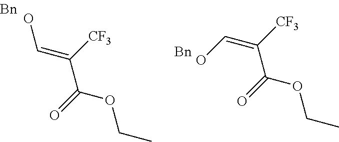

To a mixture of ((benzyloxy)methyl)triphenylphosphonium chloride (20.0 g, 47.7 mmol) in tetrahydrofuran (200 mL) was added NaH (1.9 g, 47.7 mmol, 60% in mineral oil) in batches at 0° C. The resulting mixture was stirred at 0° C. for 0.5 h. Then, a solution of ethyl 3,3,3-trifluoro-2-oxopropanoate (6.50 g, 38.2 mmol) in THF (50 mL) was added dropwise. The resulting mixture was stirred at 50° C. for 15 h. The reaction mixture was quenched with water (50 mL) and extracted with ethyl acetate (3×200 mL). The combined organic layers were dried over anhydrous sodium sulfate and concentrated under vacuum. The residue was purified by column chromatography on silica gel using 90% petroleum ether and 10% ethyl acetate as eluent to afford a mixture of ethyl (Z)-3-(benzyloxy)-2-(trifluoromethyl)acrylate and ethyl (E)-3-(benzyloxy)-2-(trifluoromethyl)acrylate (2.5 g, 10% yield) as a light-yellow oil. LC-MS: m/z 275 [M+H]⁺.

### Step 3: ethyl 1-benzyl-4-(benzyloxy)-3-(trifluoromethyl)pyrrolidine-3-carboxylate

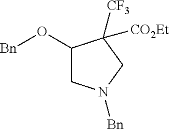 [SMILES: CCOC(=O)C1(C(F)(F)F)CN(Cc2ccccc2)CC1OCc1ccccc1]

To a stirred mixture of ethyl (Z)-3-(benzyloxy)-2-(trifluoromethyl)acrylate and ethyl (E)-3-(benzyloxy)-2-(trifluoromethyl)acrylate (3.6 g, 13.1 mmol) in N-(methoxymethyl)-1-phenyl-N-(trimethylsilylmethyl)methanamine (4.7 g, 19.7 mmol) was added TFA (150 mg, 1.3 mmol) dropwise at 0° C. The resulting mixture was stirred at 25° C. for 2 h. The reaction mixture was quenched with water (100 mL) and extracted with ethyl acetate (3×100 mL). The combined organic layers were dried over anhydrous sodium sulfate and concentrated under vacuum. The residue was purified by column chromatography on silica gel using 90% petroleum ether and 10% ethyl acetate as eluent to afford ethyl 1-benzyl-4-benzyloxy-3-(trifluoromethyl)pyrrolidine-3-carboxylate (3.6 g, 68% yield) as a light yellow oil. LC-MS: m/z 408 [M+H]⁺.

### Step 4: (1-benzyl-4-(benzyloxy)-3-(trifluoromethyl)pyrrolidin-3-yl)methanol

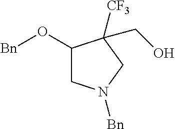 [SMILES: OCC1(C(F)(F)F)CN(Cc2ccccc2)CC1OCc1ccccc1]

To a stirred solution of ethyl 1-benzyl-4-(benzyloxy)-3-(trifluoromethyl)pyrrolidine-3-carboxylate (2.8 g, 6.8 mmol) in tetrahydrofuran (20 mL) was added LiAlH₄ (313 mg, 8.2 mmol) at 0° C. The reaction mixture was stirred at 25° C. for 3 h. The reaction mixture was quenched with water (10 mL) and the solid was filtered out. The filtrate was extracted with ethyl acetate (3×20 mL). The combined organic layers were washed with brine (20 mL), dried over anhydrous sodium sulfate and concentrated under vacuum. The residue was purified by column chromatography on silica gel using 75% petroleum ether and 25% ethyl acetate as eluent to afford (1-benzyl-4-(benzyloxy)-3-(trifluoromethyl)pyrrolidin-3-yl)methanol (1.9 g, 75% yield) as a yellow oil. ¹H NMR (400 MHz, methanol-d₄) δ 7.22-7.33 (m, 10H), 4.62 (d, J=12.0 Hz, 1H), 4.53 (d, J=11.6 Hz, 1H), 4.11 (t, J=6.8 Hz, 1H), 3.77 (d, J=12.0 Hz, 1H), 3.54-3.63 (m, 3H), 3.04-3.08 (m, 1H), 2.64-2.73 (m, 2H), 2.45-2.49 (m, 1H). LC-MS: m/z 366 [M+H]⁺.

### Step 5: 1-benzyl-4-(benzyloxy)-3-(methoxymethyl)-3-(trifluoromethyl)pyrrolidine

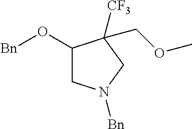 [SMILES: COCC1(C(F)(F)F)CN(Cc2ccccc2)CC1OCc1ccccc1]

To a solution of (1-benzyl-4-(benzyloxy)-3-(trifluoromethyl)pyrrolidin-3-yl)methanol (1.9 g, 5.4 mmol) in tetrahydrofuran (20 mL) was added NaH (260 mg, 6.5 mmol, 60% in mineral oil) in batches at 0° C. The reaction mixture was stirred at 0° C. for 0.5 h. Then iodomethane (1.2 g, 8.1 mmol) was added dropwise at 0° C. and the reaction mixture was stirred at 25° C. for 3 h. The reaction mixture was quenched with water (20 mL) and extracted with ethyl acetate (3×20 mL). The combined organic layers were washed with brine (50 mL), dried over anhydrous sodium sulfate and concentrated under vacuum. The residue was purified by column chromatography on silica gel using 75% petroleum ether and 25% ethyl acetate as eluent to afford 1-benzyl-4-(benzyloxy)-3-(methoxymethyl)-3-(trifluoromethyl)pyrrolidine (1.7 g, 83% yield) as a yellow oil. ¹H NMR (400 MHz, Chloroform-d) δ 7.23-7.36 (m, 10H), 4.68 (d, J=12.4 Hz, 1H), 4.54 (d, J=12.4 Hz, 1H), 4.08-4.14 (m, 1H), 3.67 (d, J=12.8 Hz, 1H), 3.57 (d, J=10.0 Hz, 1H), 3.52 (d, J=12.8 Hz, 1H), 3.35-3.38 (m, 4H), 3.12-3.16 (m, 1H), 2.71 (d, J=10.0 Hz, 1H), 2.64 (d, J=10.0 Hz, 1H), 2.46-2.51 (m, 1H). LC-MS: m/z 380 [M+H]⁺.

### Step 6: 4-(methoxymethyl)-4-(trifluoromethyl)pyrrolidin-3-ol hydrochloride

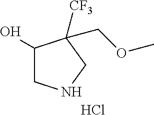

To a solution of 1-benzyl-4-benzyloxy-3-(methoxymethyl)-3-(trifluoromethyl)pyrrolidine (1.8 g, 4.7 mmol) and HCl (4.7 mL, 1M (MeOH)) in methanol (50 mL) was added Pd/C (1.8 g, 10%). The resulting mixture was stirred at 25° C. for 48 h under an atmosphere of hydrogen. The solid was filtered out. The filtrate was concentrated under vacuum to afford 4-(methoxymethyl)-4-(trifluoromethyl)pyrrolidin-3-ol hydrochloride (1.1 g, 99% yield) as a yellow solid. ¹H NMR (400 MHz, DMSO-d₆) δ 9.57 (s, 2H), 6.15 (d, J=5.2 Hz, 1H), 4.37-4.43 (m, 1H), 3.51 (s, 2H), 3.40-3.48 (m, 2H), 3.31-3.34 (m, 1H), 3.30 (s, 3H), 3.05 (dd, J=5.2, 12.0 Hz, 1H). LC-MS: m/z 200 [M+H-HCl]⁺.

### Step 7: tert-butyl 4-hydroxy-3-(methoxymethyl)-3-(trifluoromethyl)pyrrolidine-1-carboxylate

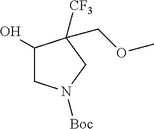 [SMILES: COCC1(C(F)(F)F)CN(C(=O)OC(C)(C)C)CC1O]

To a solution of 4-(methoxymethyl)-4-(trifluoromethyl)pyrrolidin-3-ol hydrochloride (1.1 g, 4.7 mmol) and di-tert-butyl dicarbonate (1.5 g, 7.0 mmol) in tetrahydrofuran (20 mL) was added triethylamine (2.4 g, 23.3 mmol). The resulting mixture was stirred for 3 h at 25° C. The reaction mixture was quenched with water (20 mL). The resulting solution was extracted with ethyl acetate (2×20 mL). The combined organic layers were dried over anhydrous sodium sulfate and concentrated under vacuum. The residue was purified by column chromatography on silica gel using 75% petroleum ether and 25% ethyl acetate as eluent to afford tert-butyl 4-hydroxy-3-(methoxymethyl)-3-(trifluoromethyl)pyrrolidine-1-carboxylate (1.2 g, 86% yield) as a colorless oil. LC-MS: m/z 300 [M+H]⁺.

### Step 8: tert-butyl 3-(methoxymethyl)-4-oxo-3-(trifluoromethyl)pyrrolidine-1-carboxylate

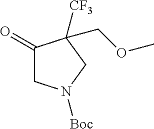 [SMILES: COCC1(C(F)(F)F)CN(C(=O)OC(C)(C)C)CC1=O]

To a mixture of tert-butyl 4-hydroxy-3-(methoxymethyl)-3-(trifluoromethyl)pyrrolidine-1-carboxylate (1.1 g, 3.7 mmol) and silica gel (360 mg) in dichloromethane (10 mL) was added PCC (360 mg, 1.7 mmol). The resulting mixture was stirred at 40° C. for 15 h. The reaction mixture was quenched with water (30 mL) and extracted with dichloromethane (3×30 mL). The combined organic layers were washed with brine (60 mL), dried over anhydrous sodium sulfate and concentrated under vacuum. The residue was purified by column chromatography on silica gel using 75% petroleum ether and 25% ethyl acetate as eluent to afford tert-butyl 3-(methoxymethyl)-4-oxo-3-(trifluoromethyl)pyrrolidine-1-carboxylate (515 mg, 31% yield) as a colorless oil. ¹H NMR (400 MHz, Chloroform-d) δ 3.79-4.14 (m, 5H), 3.57 (d, J=8.8 Hz, 1H), 3.34 (s, 3H), 1.49 (s, 9H). LC-MS: m/z 242 [M+H—C₄H₈]⁺.

### Step 9: tert-butyl (E)-2-((dimethylamino)methylene)-4-(methoxymethyl)-3-oxo-4-(trifluoromethyl)pyrrolidine-1-carboxylate

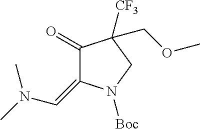

A solution of tert-butyl 3-(methoxymethyl)-4-oxo-3-(trifluoromethyl)pyrrolidine-1-carboxylate (515 mg, 1.7 mmol) in DMF-DMA (10 mL) was stirred at 35° C. for 1 h. The reaction mixture was concentrated under vacuum to afford tert-butyl (E)-2-((dimethylamino)methylene)-4-(methoxymethyl)-3-oxo-4-(trifluoromethyl)pyrrolidine-1-carboxylate (610 mg, crude) as a yellow oil. LC-MS: m/z 353 [M+H]⁺.

### Step 10: tert-butyl 2-chloro-8-(methoxymethyl)-8-(trifluoromethyl)-7,8-dihydro-6H-pyrazolo[1,5-a]pyrrolo[2,3-e]pyrimidine-6-carboxylate

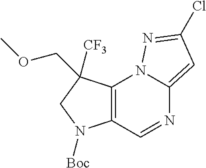 [SMILES: COCC1(C(F)(F)F)CN(C(=O)OC(C)(C)C)c2cnc3cc(Cl)nn3c21]

To a stirred solution of tert-butyl (E)-2-((dimethylamino)methylene)-4-(methoxymethyl)-3-oxo-4-(trifluoromethyl)pyrrolidine-1-carboxylate (610 mg, 1.7 mmol) in toluene (20 mL) and acetic acid (2 mL) was added 3-chloro-1H-pyrazol-5-amine (203 mg, 1.7 mmol). The resulting mixture was stirred at 95° C. for 15 h. The mixture was concentrated under vacuum. The residue was diluted with saturated aqueous NaHCO₃ solution (10 mL). The resulting mixture was extracted with ethyl acetate (3×30 mL). The combined organic layers were washed with brine (50 mL), dried over anhydrous sodium sulfate and concentrated under vacuum. The residue was purified by column chromatography on silica gel using 75% petroleum ether and 25% ethyl acetate as eluent to afford tert-butyl 2-chloro-8-(methoxymethyl)-8-(trifluoromethyl)-7,8-dihydro-6H-pyrazolo[1,5-a]pyrrolo[2,3-e]pyrimidine-6-carboxylate (45 mg, 6% yield) as a yellow oil. LC-MS: m/z 407 [M+H]⁺.

### Step 11: 2-chloro-8-(methoxymethyl)-8-(trifluoromethyl)-7,8-dihydro-6H-pyrazolo[1,5-a]pyrrolo[2,3-e]pyrimidine

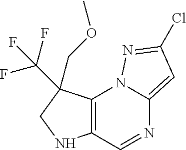 [SMILES: COCC1(C(F)(F)F)CNc2cnc3cc(Cl)nn3c21]

To a stirred solution of tert-butyl 2-chloro-8-(methoxymethyl)-8-(trifluoromethyl)-7,8-dihydro-6H-pyrazolo[1,5-a]pyrrolo[2,3-e]pyrimidine-6-carboxylate (45 mg, 110.6 μmol) in dichloromethane (4 mL) was added TFA (1 mL). The resulting mixture was stirred at 25° C. for 1 h. The mixture was concentrated under vacuum. The residue was diluted with saturated aqueous NaHCO₃ solution (5 mL). The resulting mixture was extracted with dichloromethane (3×20 mL). The combined organic layers were washed with brine (20 mL), dried over anhydrous sodium sulfate and concentrated under vacuum. The residue was purified by column chromatography on silica gel using 50% petroleum ether and 50% ethyl acetate as eluent to afford 2-chloro-8-(methoxymethyl)-8-(trifluoromethyl)-7,8-dihydro-6H-pyrazolo[1,5-a]pyrrolo[2,3-e]pyrimidine (32 mg, 90% yield) as a yellow oil. LC-MS: m/z 307 [M+H]⁺.

### Step 12: 2-chloro-N-(5-chloro-6-(2H-1,2,3-triazol-2-yl)pyridin-3-yl)-8-(methoxymethyl)-8-(trifluoromethyl)-7,8-dihydro-6H-pyrazolo[1,5-a]pyrrolo[2,3-e]pyrimidine-6-carboxamide

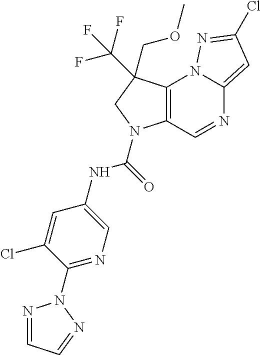 [SMILES: COCC1(C(F)(F)F)CN(C(=O)Nc2cnc(-n3nccn3)c(Cl)c2)c2cnc3cc(Cl)nn3c21]

To a stirred solution of 5-chloro-6-(2H-1,2,3-triazol-2-yl)pyridin-3-amine (27 mg, 88.0 μmol) in tetrahydrofuran (2 mL) were added triphosgene (18 mg, 60.7 μmol) and TEA (20 mg, 197.6 μmol). The resulting mixture was stirred at 40° C. for 1 h and then filtered. The filtrate was added to a solution of 2-chloro-8-(methoxymethyl)-8-(trifluoromethyl)-7,8-dihydro-6H-pyrazolo[1,5-a]pyrrolo[2,3-e]pyrimidine (34 mg, 176.1 μmol) in tetrahydrofuran (2 mL). To this solution were added N,N-dimethylpyridin-4-amine (50 mg, 409.3 μmol) and TEA (200 mg, 1.9 mmol). The mixture was stirred at 40° C. for 1 h. The mixture was concentrated under vacuum. The residue was purified by Prep-HPLC and the collected fractions were lyophilized to afford 2-chloro-N-(5-chloro-6-(2H-1,2,3-triazol-2-yl)pyridin-3-yl)-8-(methoxymethyl)-8-(trifluoromethyl)-7,8-dihydro-6H-pyrazolo[1,5-a]pyrrolo[2,3-e]pyrimidine-6-carboxamide (20 mg, 34% yield) as an off-white solid. ¹H NMR (400 MHz, methanol-d₄) δ 9.38 (s, 1H), 8.71 (d, J=2.4 Hz, 1H), 8.55 (d, J=2.4 Hz, 1H), 8.02 (s, 2H), 6.80 (s, 1H), 4.76 (d, J=11.6 Hz, 1H), 4.64 (d, J=9.2 Hz, 1H), 4.59 (d, J=11.6 Hz, 1H), 4.07 (d, J=9.2 Hz, 1H), 3.38 (s, 3H). LC-MS: m/z 528 [M+H]⁺.

### Step 13: Separation of enantiomers to obtain (R)-2-chloro-N-(5-chloro-6-(2H-1,2,3-triazol-2-yl)pyridin-3-yl)-8-(methoxymethyl)-8-(trifluoromethyl)-7,8-dihydro-6H-pyrazolo[1,5-a]pyrrolo[2,3-e]pyrimidine-6-carboxamide and (S)-2-chloro-N-(5-chloro-6-(2H-1,2,3-triazol-2-yl)pyridin-3-yl)-8-(methoxymethyl)-8-(trifluoromethyl)-7,8-dihydro-6H-pyrazolo[1,5-a]pyrrolo[2,3-e]pyrimidine-6-carboxamide

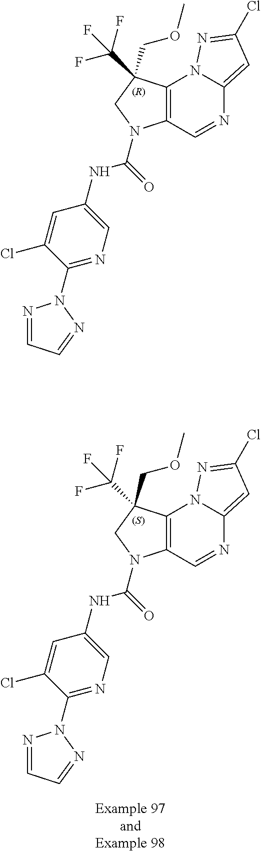

A sample containing 2-chloro-N-(5-chloro-6-(2H-1,2,3-triazol-2-yl)pyridin-3-yl)-8-(methoxymethyl)-8-(trifluoromethyl)-7,8-dihydro-6H-pyrazolo[1,5-a]pyrrolo[2,3-e]pyrimidine-6-carboxamide (20 mg) was submitted to chiral-HPLC: Column: CHIRALPAK IC, 2×25 cm, 5 um; Mobile Phase A: Hex (0.5% 2M NH₃-MeOH)-HPLC, Mobile Phase B:EtOH-HPLC; Flow rate: 20 mL/min; Gradient: 30 B to 30 B in 13 min; 220/254 nm; RT1:9.734; RT2:10.518; Injection Volume: 0.3 ml; Number Of Runs: 10. The first eluting isomer was concentrated and lyophilized to afford Example 97 (3.8 mg, 19% yield) as an off-white solid. The second eluting isomer was concentrated and lyophilized to afford Example 98 (3.8 mg, 19% yield) as an off-white solid.

Example 97: ¹HNMR (400 MHz, methanol-d₄) δ 9.38 (s, 1H), 8.71 (d, J=2.4 Hz, 1H), 8.55 (d, J=2.4 Hz, 1H), 8.02 (s, 2H), 6.80 (s, 1H), 4.76 (d, J=11.2 Hz, 1H), 4.64 (d, J=9.2 Hz, 1H), 4.59 (d, J=11.6 Hz, 1H), 4.07 (d, J=9.2 Hz, 1H), 3.38 (s, 3H). LC-MS: m/z 528 [M+H]⁺.

Example 98: ¹HNMR (400 MHz, methanol-d₄) δ 9.38 (s, 1H), 8.71 (d, J=2.4 Hz, 1H), 8.55 (d, J=2.4 Hz, 1H), 8.02 (s, 2H), 6.80 (s, 1H), 4.76 (d, J=11.2 Hz, 1H), 4.64 (d, J=9.2 Hz, 1H), 4.59 (d, J=11.6 Hz, 1H), 4.07 (d, J=9.2 Hz, 1H), 3.38 (s, 3H). LC-MS: m/z 528 [M+H]⁺.

### Method Q3

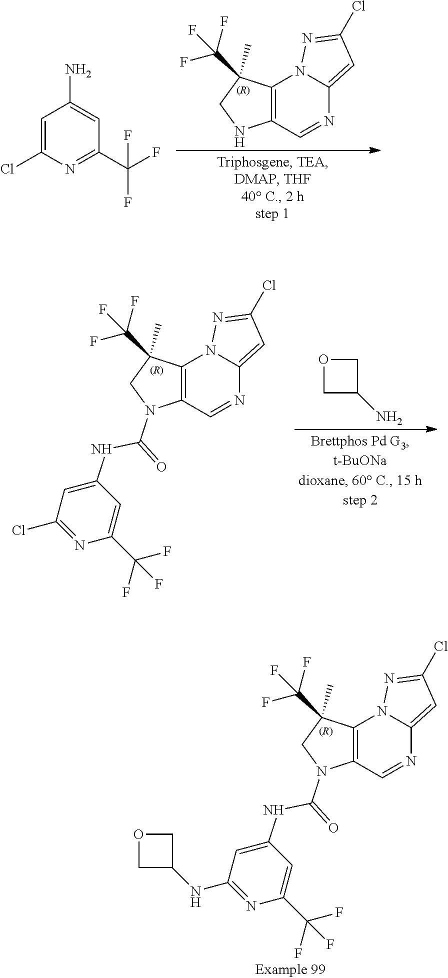

### Examples 99: (R)-2-chloro-8-methyl-N-(2-(oxetan-3-ylamino)-6-(trifluoromethyl)pyridin-4-yl)-8-(trifluoromethyl)-7,8-dihydro-6H-pyrazolo[1,5-a]pyrrolo[2,3-e]pyrimidine-6-carboxamide

### Step 1: (R)-2-chloro-N-(2-chloro-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

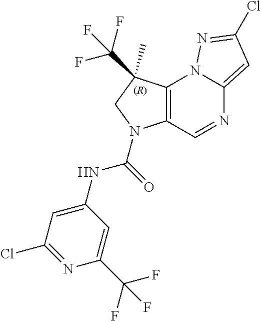

To a stirred solution of 2-chloro-6-(trifluoromethyl)pyridin-4-amine (200 mg, 1.0 mmol) in tetrahydrofuran (10 mL) were added triphosgene (181 mg, 610.5 μmol) and TEA (154 mg, 1.5 mmol). The resulting mixture was stirred at 25° C. for 0.5 h and then filtered. The filtrate was added to a solution of Method M1 isomer 2 (225 mg, 814.0 μmol). To this solution were then added TEA (1.0 g, 10.2 mmol) and N,N-dimethylpyridin-4-amine (248 mg, 2.0 mmol). The mixture was stirred at 40° C. for 2 h. The reaction mixture was concentrated. The residue was purified by column chromatography on silica gel using 90% dichloromethane and 10% methanol as eluent to afford (R)-2-chloro-N-(2-chloro-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide (350 mg, 69% yield) as a yellow solid. ¹H NMR (400 MHz, DMSO-d₆) δ 9.93 (s, 1H), 9.31 (s, 1H), 7.99-8.05 (m, 2H), 7.08 (s, 1H), 4.82-4.85 (m, 1H), 4.25-4.28 (m, 1H), 1.95 (s, 3H). LC-MS: m/z 499 [M+H]⁺.

### Step 2: (R)-2-chloro-8-methyl-N-(2-(oxetan-3-ylamino)-6-(trifluoromethyl)pyridin-4-yl)-8-(trifluoromethyl)-7,8-dihydro-6H-pyrazolo[1,5-a]pyrrolo[2,3-e]pyrimidine-6-carboxamide

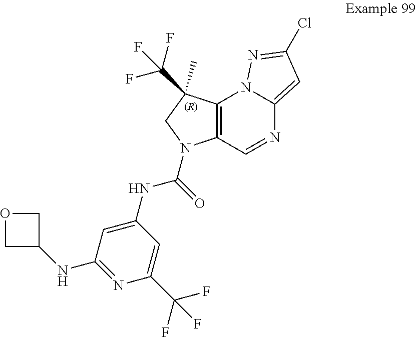

To a stirred mixture of (R)-2-chloro-N-(2-chloro-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide (350 mg, 721.1 μmol) and t-BuONa (208 mg, 2.1 mmol) in dioxane (40 mL) were added oxetan-3-amine (53 mg, 721.1 μmol) and Brettphos Pd G3 (130.7 mg, 144.2 μmol) at 25° C. under nitrogen. The reaction mixture was stirred at 60° C. for 15 h. The reaction mixture was cooled to 25° C. and concentrated. The residue was purified by column chromatography on silica gel using 40% petroleum ether and 60% ethyl acetate as eluent to afford a crude product. The crude product was submitted to Prep-HPLC purification and the collected fractions were lyophilized to give (R)-2-chloro-8-methyl-N-(2-(oxetan-3-ylamino)-6-(trifluoromethyl)pyridin-4-yl)-8-(trifluoromethyl)-7,8-dihydro-6H-pyrazolo[1,5-a]pyrrolo[2,3-e]pyrimidine-6-carboxamide (11.8 mg, 3% yield) as a yellow solid. The enantiomer of Example 99 can be prepared analogously using Method M1 isomer 1 in Method Q3, Step 1.

Examples 99: ¹H NMR (300 MHz, DMSO-d₆) δ 9.31-9.32 (m, 2H), 7.80 (d, J=5.7 Hz, 1H), 7.19 (s, 1H), 7.12 (s, 1H), 7.04 (s, 1H), 4.75-4.88 (m, 4H), 4.42-4.45 (m, 2H), 4.21-4.24 (m, 1H), 1.94 (s, 3H). LC-MS: m/z 536 [M+H]⁺.

### Example 100: (R)-2-chloro-N-(3-fluoro-2-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

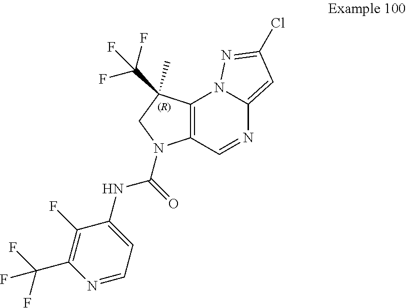

The title compound was prepared according to Method M2, Step 4 by using (3-fluoro-2-(trifluoromethyl)isonicotinic acid and Method M1 isomer 2. The enantiomer of Example 100 can be prepared analogously using Method M1 isomer 1.

Example 100: ¹H NMR (400 MHz, Methanol-d₄) δ: 9.34 (s, 1H), 8.42 (d, J=5.6 Hz, 1H), 8.32-8.35 (m, 1H), 6.83 (s, 1H), 4.89-4.98 (m, 1H), 4.29 (d, J=11.6 Hz, 1H), 2.05 (s, 3H). LC-MS: m/z 483 [M+H]⁺.

### Method R3

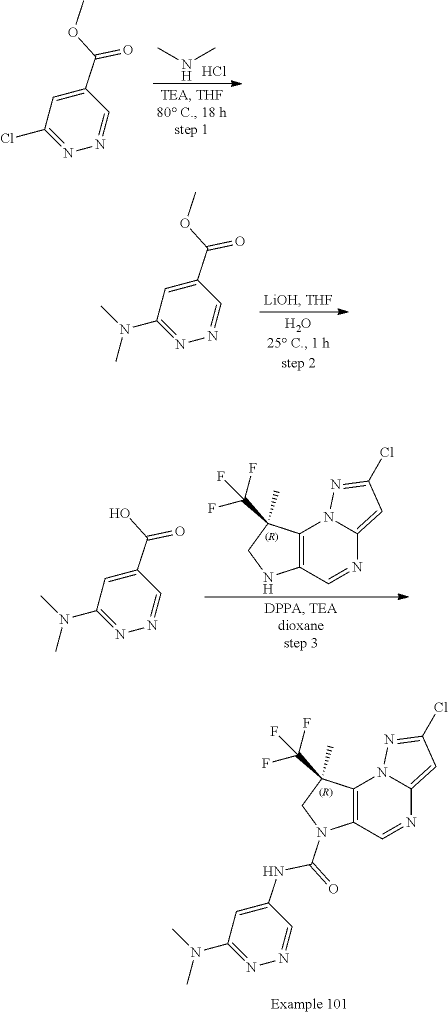

### Example 101: (R)-2-chloro-N-(6-(dimethylamino)pyridazin-4-yl)-8-methyl-8-(trifluoromethyl)-7,8-dihydro-6H-pyrazolo[1,5-a]pyrrolo[2,3-e]pyrimidine-6-carboxamide

### Step 1: methyl 6-(dimethylamino)pyridazine-4-carboxylate

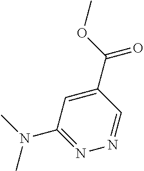

To a solution of methyl 6-chloropyridazine-4-carboxylate (5.0 g, 28.0 mmol) in tetrahydrofuran (100 mL) were added dimethylamine hydrogen chloride salt (2.84 g, 34.8 mmol) and TEA (5.9 g, 57.9 mmol). The reaction mixture was stirred at 80° C. for 18 h. The reaction mixture was concentrated under vacuum. The resulting mixture was diluted with water (100 mL) and extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate to afford methyl 6-(dimethylamino)pyridazine-4-carboxylate (800 mg, 15% yield) as a light-yellow solid. ¹H NMR (300 MHz, DMSO-d₆) δ: 8.81 (s, 1H), 7.34 (s, 1H), 3.91 (s, 3H), 3.16 (s, 6H). LC-MS: m/z 182 [M+H]⁺.

### Step 2: 6-(dimethylamino)pyridazine-4-carboxylic acid

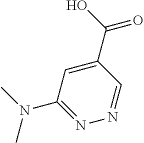

To a mixture of methyl 6-(dimethylamino)pyridazine-4-carboxylate (300 mg, 1.6 mmol) in tetrahydrofuran (2.5 mL) and water (2.5 mL) was added LiOH (79 mg, 3.3 mmol). The reaction mixture was stirred at 25° C. for 1 h. The pH was adjusted to 3 with hydrochloric acid (1 M). The resulting solution was extracted with ethyl acetate (3×10 mL). The combined organic layers were dried over anhydrous sodium sulfate and concentrated under vacuum to afford 6-(dimethylamino)pyridazine-4-carboxylic acid (400 mg) as a light-yellow solid. LC-MS: m/z 168 [M+H]⁺.

### Step 3: (R)-2-chloro-N-(6-(dimethylamino)pyridazin-4-yl)-8-methyl-8-(trifluoromethyl)-7,8-dihydro-6H-pyrazolo[1,5-a]pyrrolo[2,3-e]pyrimidine-6-carboxamide

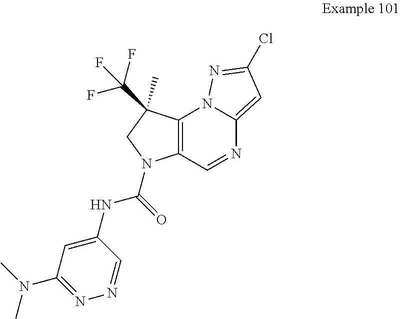 [SMILES: CN(C)c1cc(NC(=O)N2C[C@@](C)(C(F)(F)F)c3c2cnc2cc(Cl)nn32)cnn1]

To a solution of 6-(dimethylamino)pyridazine-4-carboxylic acid (100 mg, 598.2 μmol) in dioxane (2 mL) were added DPPA (197 mg, 717.8 μmol), triethylamine (302 mg, 3.0 mmol) and Method M1 isomer 2 (164 mg, 598.2 μmol). The mixture was stirred at 100° C. for 2 h. The mixture was concentrated under vacuum. The residue was purified by Prep-HPLC and the collected fractions were lyophilized to afford (R)-2-chloro-N-(6-(dimethylamino)pyridazin-4-yl)-8-methyl-8-(trifluoromethyl)-7,8-dihydro-6H-pyrazolo[1,5-a]pyrrolo[2,3-e]pyrimidine-6-carboxamide (17 mg, 6% yield) as a white solid. The enantiomer of Example 101 can be prepared analogously using Method M1 isomer 1.

Example 101: ¹H NMR (300 MHz, DMSO-d₆) δ: 9.34 (s, 2H), 8.75 (d, J=1.5 Hz, 1H), 7.27 (d, J=1.8 Hz, 1H), 7.08 (s, 1H), 4.86 (d, J=11.7 Hz, 1H), 4.29 (d, J=11.4 Hz, 1H), 3.09 (s, 6H), 1.97 (s, 3H). LC-MS: m/z 441 [M+H]⁺.

### Method S3

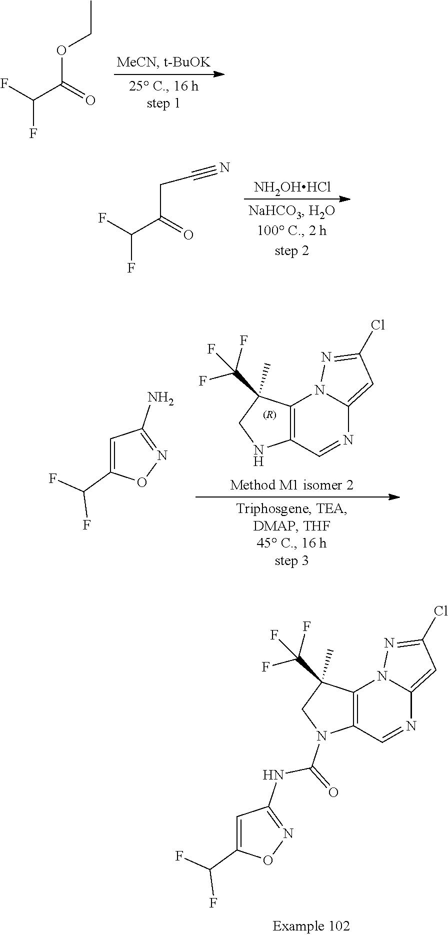

### Example 102: (R)-2-chloro-N-(5-(difluoromethyl)isoxazol-3-yl)-8-methyl-8-(trifluoro methyl)-7,8-dihydro-6H-pyrazolo[1,5-a]pyrrolo[2,3-e]pyrimidine-6-carboxamide

### Step 1: 4,4-difluoro-3-oxobutanenitrile

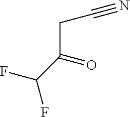

To a stirred solution of methyl 2,2-difluoroacetate (10.0 g, 90.8 mmol) in tetrahydrofuran (50 mL) were added t-BuOK (20.2 g, 181.9 mmol) and acetonitrile (3.6 g, 90.8 mmol). The reaction mixture was stirred at 25° C. for 16 h. The reaction mixture was quenched by the addition of water (500 mL). The resulting solution was extracted with diethyl ether (3×300 mL). The combined organic layers were dried over anhydrous sodium sulfate and concentrated under vacuum. This resulted in 4,4-difluoro-3-oxobutanenitrile (5.0 g, 45% yield) as a colorless oil. LC-MS: m/z 120 [M+H]⁺.

### Step 2: 5-(difluoromethyl)isoxazol-3-amine

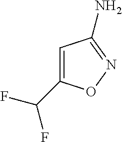

To a stirred solution of NaHCO₃ (7.0 g, 83.9 mmol) in water (30 mL) was added hydroxylamine hydrochloride (8.7 g, 125.9 mmol). The mixture was stirred at 25° C. for 1 h. The reaction solution was added 4,4-difluoro-3-oxobutanenitrile (10.0 g, 83.9 mmol). The reaction mixture was stirred at 100° C. for 2 h under nitrogen. The mixture was allowed to cool down to 25° C. The reaction mixture was quenched by the addition of water (200 mL). The resulting solution was extracted with ethyl acetate (3×200 mL), dried over anhydrous sodium sulfate and concentrated under vacuum. The residue was purified by column chromatography using 50% petroleum ether and 50% ethyl acetate as eluent to afford 5-(difluoromethyl)isoxazol-3-amine (250 mg, 2% yield) as a colorless oil. ¹H NMR (400 MHz, Chloroform-d) δ: 6.57 (t, J=56.0 Hz, 1H), 5.33 (s, 1H), 4.63 (br, 2H). LC-MS: m/z 135 [M+H]⁺.

### Step 3: (R)-2-chloro-N-(5-(difluoromethyl)isoxazol-3-yl)-8-methyl-8-(trifluoromethyl)-7,8-dihydro-6H-pyrazolo[1,5-a]pyrrolo[2,3-e]pyrimidine-6-carboxamide

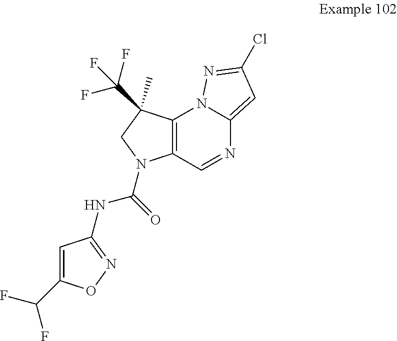 [SMILES: C[C@@]1(C(F)(F)F)CN(C(=O)Nc2cc(C(F)F)on2)c2cnc3cc(Cl)nn3c21]

To a stirred solution of Method M1 isomer 2 (50 mg, 204.2 μmol) in tetrahydrofuran (5 mL) were added triphosgene (36 mg, 102.1 μmol) and TEA (22 mg, 205.4 μmol) at 0° C. The resulting mixture was stirred at 25° C. for 0.5 h and then filtered. The filtrate was added to a solution of 5-(difluoromethyl)isoxazol-3-amine (54 mg, 408.2 μmol) in tetrahydrofuran (2 mL). To this solution was then added N,N-dimethylpyridin-4-amine (48 mg, 404.6 μmol) and TEA (204 mg, 2.0 mmol). The mixture was stirred at 45° C. for 16 h. The mixture was poured into water (20 mL) and extracted with ethyl acetate (3×20 mL). The combined organic layers were washed with brine (20 mL), dried over anhydrous sodium sulfate and concentrated under vacuum. The residue was purified by Prep-HPLC and the collected fractions were lyophilized to give (R)-2-chloro-N-(5-(difluoromethyl)isoxazol-3-yl)-8-methyl-8-(trifluoromethyl)-7,8-dihydro-6H-pyrazolo[1,5-a]pyrrolo[2,3-e]pyrimidine-6-carboxamide (20 mg, 11% yield) as a white solid. The enantiomer of Example 102 can be prepared analogously using Method M1 isomer 1.

Example 102: ¹H NMR (400 MHz, DMSO-d₆) δ:11.33 (s, 1H), 9.32 (s, 1H), 7.19 (t, J=53.2 Hz, 1H), 7.06 (s, 1H), 6.40 (s, 1H), 4.81 (d, J=11.6 Hz, 1H), 4.22 (d, J=11.6 Hz, 1H), 1.94 (s, 3H). LC-MS: m/z 437 [M+H]⁺.

### Method T3

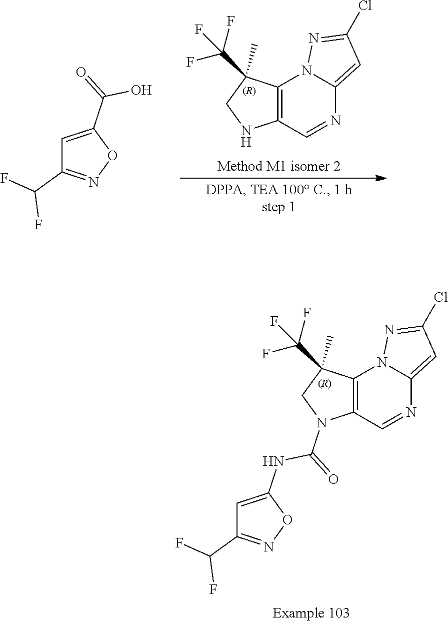

### Example 103: (R)-2-chloro-N-(3-(difluoromethyl)isoxazol-5-yl)-8-methyl-8-(trifluoromethyl)-7,8-dihydro-6H-pyrazolo[1,5-a]pyrrolo[2,3-e]pyrimidine-6-carboxamide

### Step 1: (R)-2-chloro-N-(3-(difluoromethyl)isoxazol-5-yl)-8-methyl-8-(trifluoromethyl)-7,8-dihydro-6H-pyrazolo[1,5-a]pyrrolo[2,3-e]pyrimidine-6-carboxamide

To a solution of 3-(difluoromethyl)isoxazole-5-carboxylic acid (100 mg, 613 μmol) in dioxane (2 mL) were added DPPA (179 mg, 736 μmol), TEA (310 mg, 3.1 mmol) and Method M1 isomer 2 (170 mg, 613 μmol). The mixture was stirred at 100° C. for 2 h. The mixture was concentrated under vacuum. The residue was purified by Prep-HPLC and the collected fractions were lyophilized to afford (R)-2-chloro-N-(3-(difluoromethyl)isoxazol-5-yl)-8-methyl-8-(trifluoromethyl)-7,8-dihydro-6H-pyrazolo[1,5-a]pyrrolo[2,3-e]pyrimidine-6-carboxamide (17.5 mg, 6.5% yield) as a white solid. The enantiomer of Example 103 can be prepared analogously using Method M1 isomer 1.

Example 103: ¹H NMR (400 MHz, DMSO-d₆) δ: 11.34 (s, 1H), 9.33 (s, 1H), 7.19 (t, J=53.2 Hz, 1H), 7.07 (s, 1H), 6.40 (s, 1H), 4.82 (d, J=11.6 Hz, 1H), 4.23 (d, J=12.0 Hz, 1H), 1.95 (s, 3H). LC-MS: m/z 437 [M+H]⁺.

### Method U3

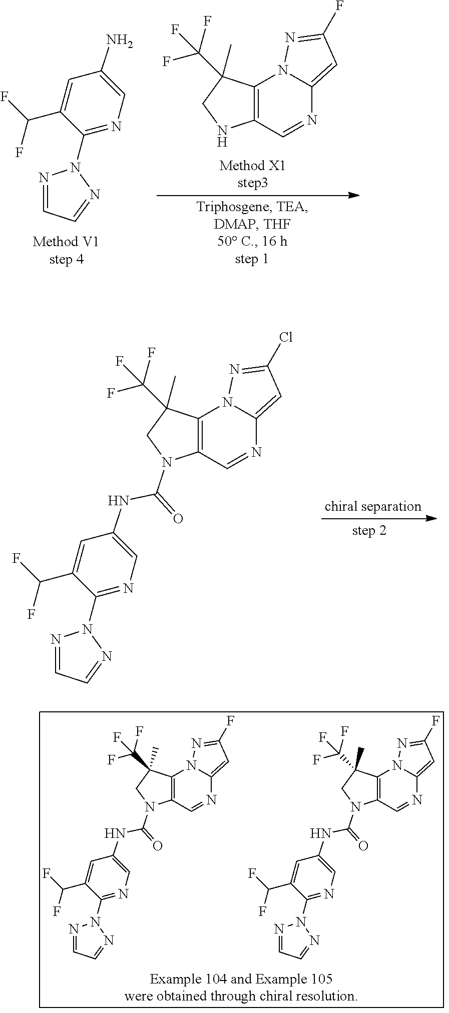 [SMILES: Nc1cnc(-n2nccn2)c(C(F)F)c1 ; CC1(C(F)(F)F)CNc2cnc3cc(F)nn3c21 ; CC1(C(F)(F)F)CN(C(=O)Nc2cnc(-n3nccn3)c(C(F)F)c2)c2cnc3cc(Cl)nn3c21 ; C[C@@]1(C(F)(F)F)CN(C(=O)Nc2cnc(-n3nccn3)c(C(F)F)c2)c2cnc3cc(F)nn3c21 ; C[C@]1(C(F)(F)F)CN(C(=O)Nc2cnc(-n3nccn3)c(C(F)F)c2)c2cnc3cc(F)nn3c21]

### Example 104 and 105: Single enantiomers obtained from a racemic mixture containing (R)—N-(5-(difluoromethyl)-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide and (S)—N-(5-(difluoromethyl)-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

### Step 1: N-(5-(difluoromethyl)-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

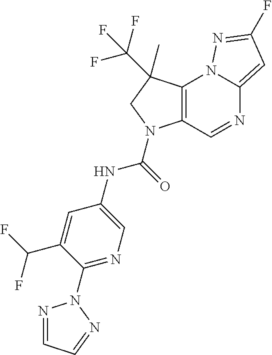 [SMILES: CC1(C(F)(F)F)CN(C(=O)Nc2cnc(-n3nccn3)c(C(F)F)c2)c2cnc3cc(F)nn3c21]

To a stirred solution of 2-fluoro-8-methyl-8-(trifluoromethyl)-7,8-dihydro-6H-pyrazolo[1,5-a]pyrrolo[2,3-e]pyrimidine (Method X1 step 3; 111 mg, 426.2 μmol) in tetrahydrofuran (2 mL) were added triphosgene (84 mg, 284.1 μmol) and TEA (72 mg, 710.3 μmol). The resulting mixture was stirred at 25° C. for 0.5 h and then filtered. The filtrate was added to a solution of 5-(difluoromethyl)-6-(2H-1,2,3-triazol-2-yl)pyridin-3-amine (Method V1 step 4; 100 mg, 473.6 μmol) in tetrahydrofuran (1 mL). To this solution were added N,N-dimethylpyridin-4-amine (87 mg, 710.3 μmol) and TEA (479 mg, 4.7 mmol). The mixture was stirred at 50° C. for 16 h. The mixture was concentrated under vacuum. The residue was purified by Prep-HPLC and the collected fractions were lyophilized to afford N-(5-(difluoromethyl)-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide (100 mg, 47% yield) as a white solid. LC-MS: m/z 498 [M+H]⁺.

### Step 2: Separation of enantiomers to obtain (R)—N-(5-(difluoromethyl)-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide and (S)—N-(5-(difluoromethyl)-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

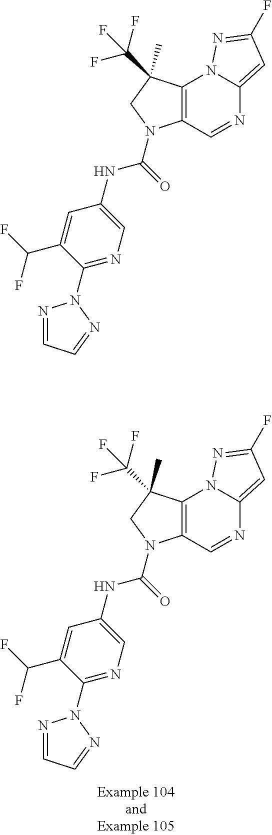 [SMILES: C[C@@]1(C(F)(F)F)CN(C(=O)Nc2cnc(-n3nccn3)c(C(F)F)c2)c2cnc3cc(F)nn3c21 ; C[C@]1(C(F)(F)F)CN(C(=O)Nc2cnc(-n3nccn3)c(C(F)F)c2)c2cnc3cc(F)nn3c21]

100 mg of N-(5-(difluoromethyl)-6-(2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide was submitted to chiral-HPLC: Column: Lux 5u Cellulose-4, 2.12*25 cm, 5 um; Mobile Phase A: Hex(0.5% 2M NH₃-MeOH)—HPLC, Mobile Phase B:EtOH—HPLC; Flow rate: 20 mL/min; Gradient: 10 B to 10 B in 21 min; 220/254 nm; RT1:12.058; RT2:17.004; Injection Volume: 1.5 ml; Number Of Runs: 4. The first eluting isomer was concentrated and lyophilized to afford Example 105 (26.9 mg, 11% yield) as a white solid. The second eluting isomer was concentrated and lyophilized to afford Example 104 (21.8 mg, 9% yield) as a white solid. Examples 104 and 105 are enantiomers, but their absolute stereochemistry is not yet known.

Example 104: ¹H NMR (400 MHz, Chloroform-d) δ: 9.41 (s, 1H), 8.76 (s, 1H), 8.61 (s, 1H), 7.96 (s, 2H), 7.55 (t, J=54.8 Hz, 1H), 6.90 (s, 1H), 6.35 (d, J=5.2 Hz, 1H), 4.63 (d, J=10.4 Hz, 1H), 4.09 (d, J=10.4 Hz, 1H), 2.05 (s, 3H). LC-MS: m/z 498 [M+H]⁺.

Example 105: ¹H NMR (400 MHz, Chloroform-d) δ: 9.42 (s, 1H), 8.77 (s, 1H), 8.61 (s, 1H), 7.96 (s, 2H), 7.56 (t, J=54.8 Hz, 1H), 6.78 (s, 1H), 6.36 (d, J=5.2 Hz, 1H), 4.62 (d, J=10.4 Hz, 1H), 4.09 (d, J=10.4 Hz, 1H), 2.06 (s, 3H). LC-MS: m/z 498 [M+H]⁺.

### Method V3

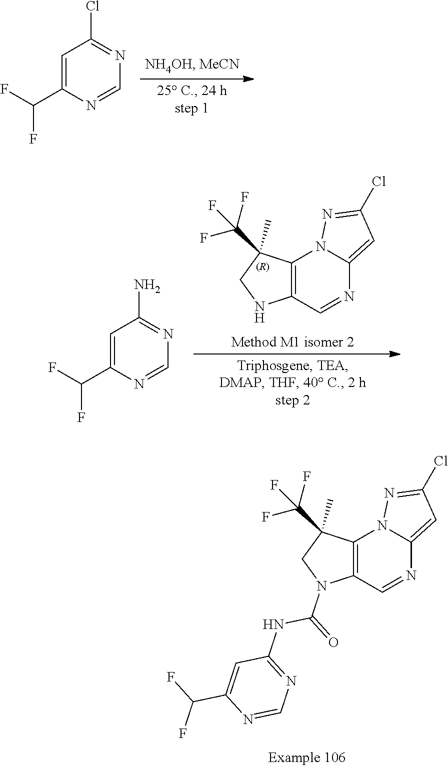

### Example 106: (R)-2-chloro-N-(6-(difluoromethyl)pyrimidin-4-yl)-8-methyl-8-(trifluoromethyl)-7,8-dihydro-6H-pyrazolo[1,5-a]pyrrolo[2,3-e]pyrimidine-6-carboxamide

### Step 1: 6-(difluoromethyl)pyrimidin-4-amine

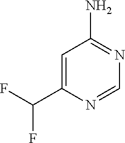

To a stirred solution of 4-chloro-6-(difluoromethyl)pyrimidine (200 mg, 1.2 mmol) in acetonitrile (2 mL) was added ammonium hydroxide (1 mL). The reaction mixture was stirred at 25° C. for 24 h. The reaction mixture was concentrated under vacuum. The resulting mixture was diluted with water (10 mL) and extracted with ethyl acetate (3×10 mL). The combined organic layers were dried over anhydrous sodium sulfate and concentrated under vacuum to afford 6-(difluoromethyl)pyrimidin-4-amine (160 mg, 90% yield) as a light-yellow solid. ¹HNMR (400 MHz, DMSO-d₆) δ: 8.42 (s, 1H), 7.28 (s, 2H), 6.71 (t, J=54.8 Hz, 1H), 6.64 (s, 1H). LC-MS: m/z 146 [M+H]⁺.

### Step 2: (R)-2-chloro-N-(6-(difluoromethyl)pyrimidin-4-yl)-8-methyl-8-(trifluoromethyl)-7,8-dihydro-6H-pyrazolo[1,5-a]pyrrolo[2,3-e]pyrimidine-6-carboxamide

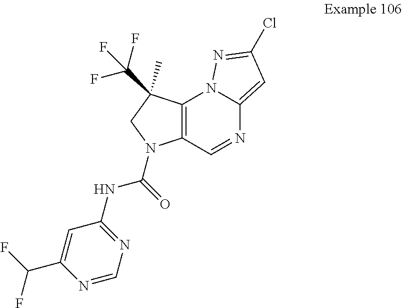 [SMILES: C[C@@]1(C(F)(F)F)CN(C(=O)Nc2cc(C(F)F)ncn2)c2cnc3cc(Cl)nn3c21]

To a stirred solution of 6-(difluoromethyl)pyrimidin-4-amine (63 mg, 434 μmol) in tetrahydrofuran (5 mL) were added triphosgene (86 mg, 289 μmol) and TEA (58 mg, 578 μmol) at 0° C. The resulting mixture was stirred at 25° C. for 1 h and then filtered. The filtrate was added to a solution of Method M1 isomer 2 (80 mg, 289 μmol) in tetrahydrofuran (1 mL). To this solution was then added TEA (293 mg, 2.9 mmol) and N,N-dimethylpyridin-4-amine (71 mg, 578 μmol). The mixture was stirred at 40° C. for 2 h. The mixture was concentrated under vacuum. The residue was purified by Prep-HPLC and the collected fractions were lyophilized to give (R)-2-chloro-N-(6-(difluoromethyl)pyrimidin-4-yl)-8-methyl-8-(trifluoromethyl)-7,8-dihydro-6H-pyrazolo[1,5-a]pyrrolo[2,3-e]pyrimidine-6-carboxamide (15.3 mg, 12% yield) as a light-yellow solid. The enantiomer of Example 106 can be prepared analogously using Method M1 isomer 1.

Example 106: ¹H NMR (300 MHz, DMSO-d₆): 10.71 (s, 1H), 9.32 (s, 1H), 9.01 (s, 1H), 8.16 (s, 1H), 7.07 (s, 1H), 6.98 (t, J=54.6 Hz, 1H), 5.01 (d, J=11.7 Hz, 1H), 4.28 (d, J=11.7 Hz, 1H), 1.94 (s, 3H). LC-MS: m/z 448 [M+H]⁺.

### Method W3

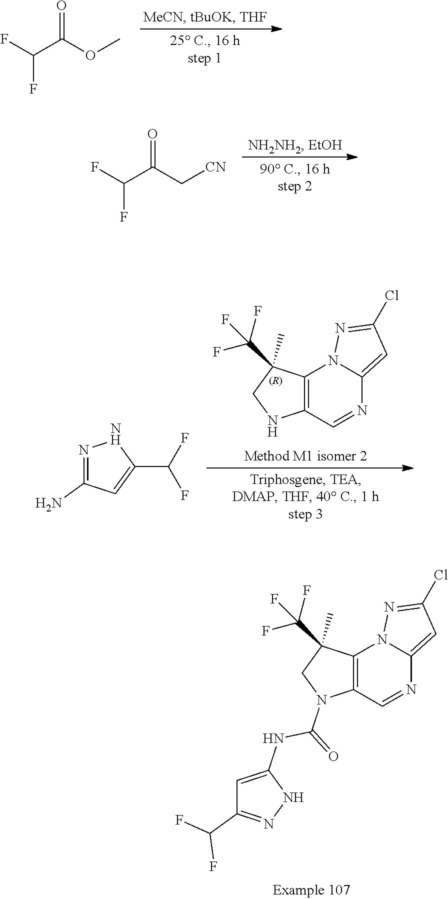

### Examples 107: (R)-2-chloro-N-(3-(difluoromethyl)-1H-pyrazol-5-yl)-8-methyl-8-(trifluoromethyl)-7,8-dihydro-6H-pyrazolo[1,5-a]pyrrolo[2,3-e]pyrimidine-6-carboxamide

### Step 1: 4,4-difluoro-3-oxobutanenitrile

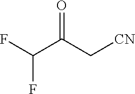

To a stirred solution of methyl 2,2-difluoroacetate (5.0 g, 45.4 mmol) in tetrahydrofuran (50 mL) was added t-BuOK (10.2 g, 90.9 mmol) and acetonitrile (1.8 g, 45.4 mmol). The reaction mixture was stirred at 25° C. for 16 h. The reaction mixture was quenched with water (200 mL). The resulting solution was extracted with diethyl ether (3×300 mL). The combined organic layers were dried over anhydrous sodium sulfate and concentrated under vacuum. The combined organic layers were concentrated under vacuum to give 4,4-difluoro-3-oxobutanenitrile (2.5 g, 45% yield) as a colorless oil. LC-MS: m/z 120 [M+H]⁺.

### Step 2: 5-(difluoromethyl)-1H-pyrazol-3-amine

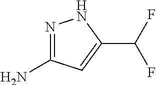

To a stirred solution of 4,4-difluoro-3-oxobutanenitrile (2.5 g, 21.0 mmol) in ethanol (20 mL) was added hydrazine hydrate (21 g, 42.0 mmol). The reaction mixture was stirred at 90° C. for 16 h under nitrogen. The mixture was allowed to cool down to 25° C. The reaction mixture was quenched with water (200 mL). The resulting solution was extracted with ethyl acetate (3×200 mL). The combined organic layers were dried over anhydrous sodium sulfate and concentrated under vacuum. The residue was purified by column chromatography on silica gel using 50% petroleum ether and 50% ethyl acetate as eluent to afford 5-(difluoromethyl)-1H-pyrazol-3-amine (700 mg, 25% yield) as a white solid. ¹H NMR (400 MHz, Chloroform-d) δ: 11.9 (br, 1H), 6.55 (t, d=56.0 Hz, 1H), 5.78 (s, 1H), 4.90 (br, 2H). LC-MS: m/z 134 [M+H]⁺.

### Step 3: (R)-2-chloro-N-(3-(difluoromethyl)-1H-pyrazol-5-yl)-8-methyl-8-(trifluoromethyl)-7,8-dihydro-6H-pyrazolo[1,5-a]pyrrolo[2,3-e]pyrimidine-6-carboxamide

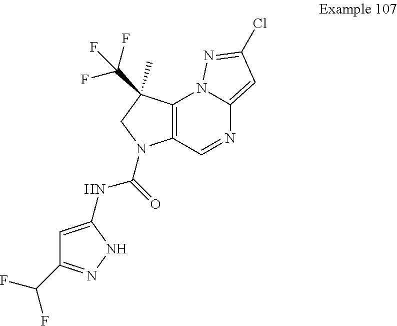 [SMILES: C[C@@]1(C(F)(F)F)CN(C(=O)Nc2cc(C(F)F)n[nH]2)c2cnc3cc(Cl)nn3c21]

To a stirred solution of 5-(difluoromethyl)-1H-pyrazol-3-amine (40 mg, 225.6 μmol) in tetrahydrofuran (8 mL) were added triphosgene (54 mg, 180.5 μmol) and TEA (22 mg, 217.4 μmol) at 25° C. The resulting mixture was stirred for 0.5 h at 28° C. and then filtered. The resulting filtrate was added to a solution of Method M1 isomer 2 (40 mg, 144.9 μmol) in tetrahydrofuran (1 mL). To this solution was then added TEA (146 mg, 1.4 mmol) and N,N-Dimethylpyridin-4-amine (36 mg, 289.9 μmol). The reaction mixture was stirred at 40° C. for 1 h. The mixture was allowed to cool down to 25° C. The reaction mixture was quenched with water (50 mL). The resulting solution was extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate as eluents to afford the crude product which was submitted to Prep-HPLC purification and the collected fractions were lyophilized to give (R)-2-chloro-N-(3-(difluoromethyl)-1H-pyrazol-5-yl)-8-methyl-8-(trifluoromethyl)-7,8-dihydro-6H-pyrazolo[1,5-a]pyrrolo[2,3-e]pyrimidine-6-carboxamide (23.2 mg, 36.8% yield) as a white solid. The enantiomer of Example 107 can be prepared analogously using Method M1 isomer 1.

Example 107: ¹H NMR (400 MHz, DMSO-d₆) δ:13.06 (br, 1H), 9.81 (br, 1H), 9.33 (s, 1H), 7.05 (s, 1H), 6.68 (t, d=56 Hz, 1H), 6.30 (s, 1H), 4.77 (d, J=8.8 Hz, 1H), 4.20 (d, J=11.6 Hz, 1H), 1.96 (s, 3H). LC-MS: m/z 436 [M+H]⁺.

### Method X3

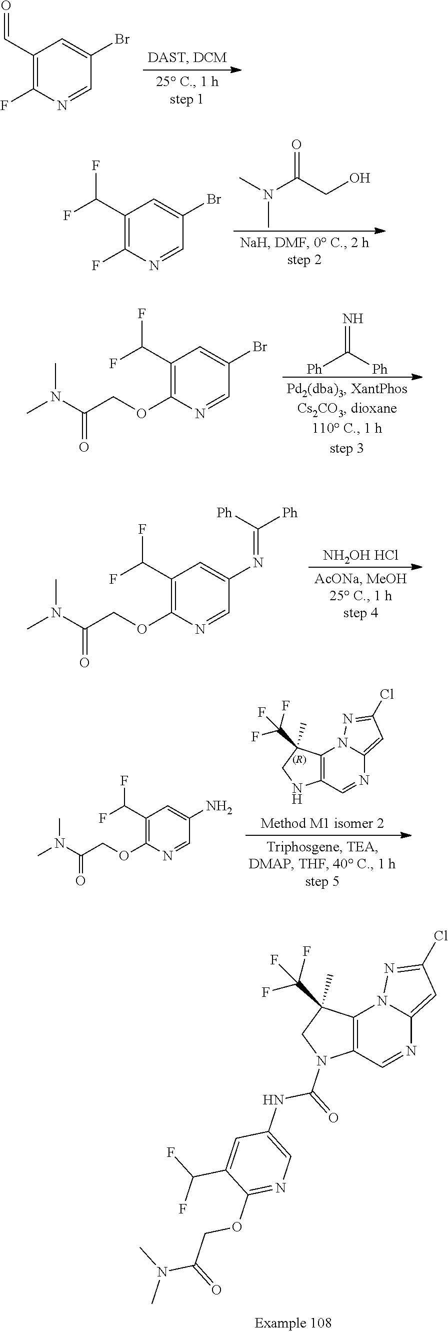

### Example 108: (R)-2-chloro-N-(5-(difluoromethyl)-6-(2-(dimethylamino)-2-oxoethoxy)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 5-bromo-3-(difluoromethyl)-2-fluoropyridine

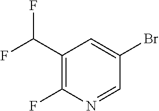 [SMILES: Fc1ncc(Br)cc1C(F)F]

To a stirred solution of 5-bromo-2-fluoronicotinaldehyde (9.5 g, 46.6 mmol) in dichloromethane (200 mL) was added DAST (15.0 g, 93.1 mmol) at −20° C. The reaction mixture was stirred at 25° C. for 1 h. The reaction solution was quenched by saturated aqueous NaHCO₃ solution (500 mL). The resulting mixture was extracted with dichloromethane (3×500 mL). The combined organic layers were dried over anhydrous sodium sulfate and concentrated under vacuum. The residue was purified by column chromatography on silica gel using 90% petroleum ether and 10% ethyl acetate as eluents to afford 5-bromo-3-(difluoromethyl)-2-fluoropyridine (8.5 g, 73% yield) as a colorless oil. ¹H NMR (400 MHz, Chloroform-d) δ: 8.42 (s, 1H), 8.16 (s, 1H), 6.83 (t, J=54.4 Hz, 1H). LC-MS: m/z 226 [M+H]⁺.

### Step 2: 2-((5-bromo-3-(difluoromethyl)pyridin-2-yl)oxy)-N,N-dimethylacetamide

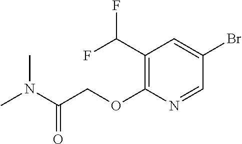 [SMILES: CN(C)C(=O)COc1ncc(Br)cc1C(F)F]

To a stirred solution of 2-hydroxy-N,N-dimethylacetamide (2.1 g, 20.1 mmol) in N,N-dimethylformamide (100 mL) was added NaH (2.2 g, 92.9 mmol, 60% in mineral oil) in batches at 0° C. The reaction mixture was stirred at 0° C. for 15 min and the solution of 5-bromo-3-(difluoromethyl)-2-fluoropyridine (3.5 g, 15.5 mmol) in N,N-dimethylformamide (10 mL) was added dropwise. The mixture was stirred at 0° C. for 2 h. The reaction mixture was quenched by water (500 mL). The resulting solution was extracted with ethyl acetate (3×500 mL). The combined organic layers were washed with brine (3×1000 mL), dried over anhydrous sodium sulfate and concentrated under vacuum. The residue was purified by column chromatography on silica gel using 70% petroleum ether and 30% ethyl acetate as eluents to afford 2-((5-bromo-3-(difluoromethyl)pyridin-2-yl)oxy)-N,N-dimethylacetamide (4.5 g, 85% yield) as a white solid. ¹H NMR (400 MHz, DMSO-d₆) δ: 8.40 (s, 1H), 8.14 (s, 1H), 7.04 (t, J=54.4 Hz, 1H), 5.15 (s, 2H), 2.96 (s, 3H), 2.79 (s, 3H). LC-MS: m/z 309 [M+H]⁺.

### Step 3: 2-((3-(difluoromethyl)-5-((diphenylmethylene)amino)pyridin-2-yl)oxy)-N,N-dimethylacetamide

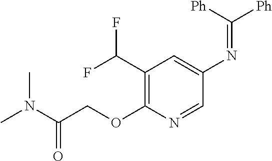 [SMILES: CN(C)C(=O)COc1ncc(N=C(c2ccccc2)c2ccccc2)cc1C(F)F]

To a stirred solution of 2-((5-bromo-3-(difluoromethyl)pyridin-2-yl)oxy)-N,N-dimethylacetamide (200 mg, 647.0 μmol) in dioxane (6 mL) were added diphenylmethanimine (234 mg, 1.3 mmol), Cs₂CO₃ (632 mg, 1.9 mmol), Xantphos (112 mg, 194.1 μmol) and Pd₂(dba)₃ (178 mg, 194.1 μmol) under nitrogen atmosphere. The reaction mixture was stirred at 110° C. for 1 h under nitrogen atmosphere. The mixture was cooled to 25° C. The solvent was removed under vacuum. The residue was purified by column chromatography on silica gel using 80% petroleum ether and 20% ethyl acetate as eluents to afford 2-((3-(difluoromethyl)-5-((diphenylmethylene)amino)pyridin-2-yl)oxy)-N,N-dimethylacetamide (200 mg, 68% yield) as a brown oil. LC-MS: m/z 410 [M+H]⁺.

### Step 4: 2-((5-amino-3-(difluoromethyl)pyridin-2-yl)oxy)-N,N-dimethylacetamide

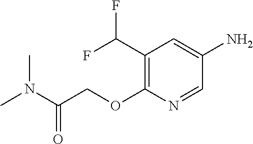 [SMILES: CN(C)C(=O)COc1ncc(N)cc1C(F)F]

To a stirred solution of 2-((3-(difluoromethyl)-5-((diphenylmethylene)amino)pyridin-2-yl)oxy)-N,N-dimethylacetamide (180 mg, 439.6 μmol) in methanol (5 mL) were added hydroxylamine hydrochloride (64 mg, 923.2 μmol) and sodium acetate (90 mg, 1.1 mmol). The reaction was stirred at 25° C. for 1 h. The solvent was removed under vacuum. The residue was purified by Prep-TLC using 90% dichloromethane and 10% methanol as eluent to afford 2-((5-amino-3-(difluoromethyl)pyridin-2-yl)oxy)-N,N-dimethylacetamide (80 mg, 67% yield) as a white solid. ¹H NMR (300 MHz, DMSO-d₆) δ: 7.56 (s, 1H), 7.22 (s, 1H), 6.98 (t, J=55.2 Hz, 1H), 5.04 (s, 2H), 4.96 (br, 2H), 2.94 (s, 3H), 2.78 (s, 3H). LC-MS: m/z 246 [M+H]⁺.

### Step 5: (R)-2-chloro-N-(5-(difluoromethyl)-6-(2-(dimethylamino)-2-oxoethoxy)pyridin-3-yl)-8-methyl-8-(trifluoromethyl)-7,8-dihydro-6H-pyrazolo[1,5-a]pyrrolo[2,3-e]pyrimidine-6-carboxamide

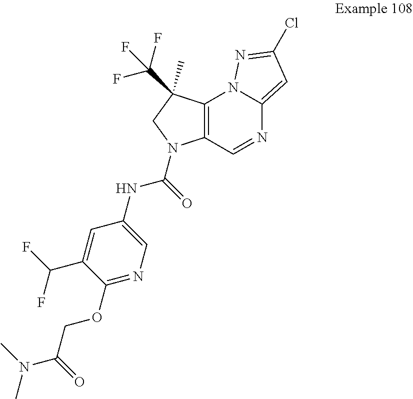 [SMILES: CN(C)C(=O)COc1ncc(NC(=O)N2C[C@@](C)(C(F)(F)F)c3c2cnc2cc(Cl)nn32)cc1C(F)F]

To a stirred solution of 2-((5-amino-3-(difluoromethyl)pyridin-2-yl)oxy)-N,N-dimethylacetamide (51 mg, 207.9 μmol) in tetrahydrofuran (5 mL) were added triphosgene (37 mg, 124.8 μmol) and TEA (32 mg, 311.9 μmol). The resulting mixture was stirred at 25° C. for 0.5 h and then filtered. The filtrate was added to a solution of Method M1 isomer 2 (40 mg, 145.6 μmol) in tetrahydrofuran (1 mL). To this solution were added N,N-dimethylpyridin-4-amine (50 mg, 415.9 μmol) and TEA (210 mg, 2.1 mmol). The mixture was stirred at 40° C. for 1 h. The mixture was concentrated under vacuum. The residue was purified by Prep-HPLC and the collected fractions were lyophilized to afford (R)-2-chloro-N-(5-(difluoromethyl)-6-(2-(dimethylamino)-2-oxoethoxy)pyridin-3-yl)-8-methyl-8-(trifluoromethyl)-7,8-dihydro-6H-pyrazolo[1,5-a]pyrrolo[2,3-e]pyrimidine-6-carboxamide (40 mg, 35% yield) as a white solid. The enantiomer of Example 108 can be prepared analogously using Method M1 isomer 1.

Example 108: ¹H NMR (300 MHz, DMSO-d₆) δ: 9.34 (s, 1H), 9.21 (br, 1H), 8.39 (s, 1H), 8.17 (s, 1H), 7.12 (t, J=54.9 Hz, 1H), 7.05 (s, 1H), 5.16 (s, 2H), 4.78 (d, J=11.4 Hz, 1H), 4.25 (d, J=11.4 Hz, 1H), 3.00 (s, 3H), 2.83 (s, 3H), 1.98 (s, 3H). LC-MS: m/z 548 [M+H]⁺.

### Method Y3

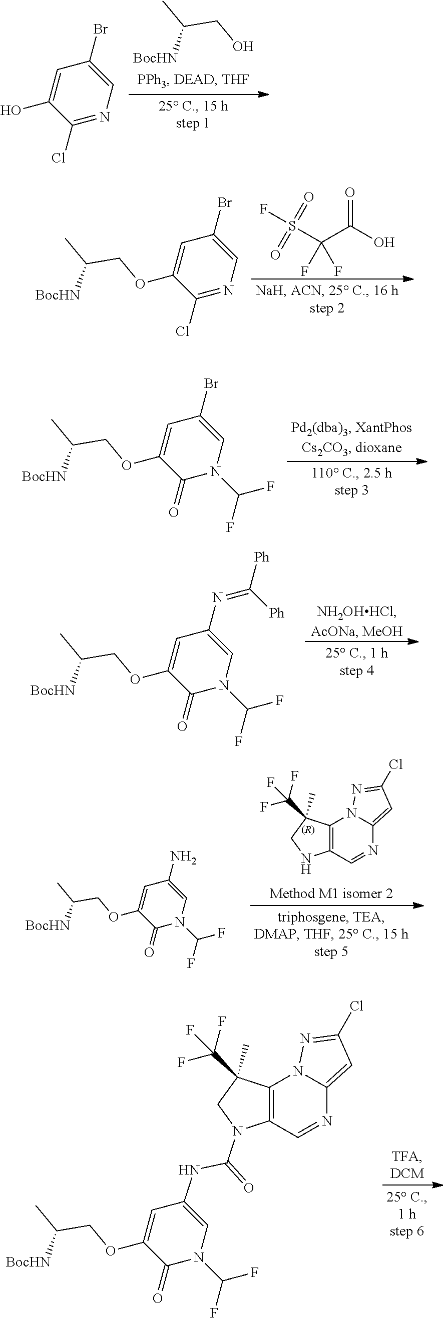

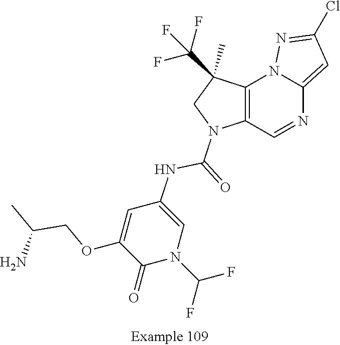 [SMILES: C[C@@H](N)COc1cc(NC(=O)N2C[C@@](C)(C(F)(F)F)c3c2cnc2cc(Cl)nn32)cn(C(F)F)c1=O]

### Example 109: (R)—N-(5-((R)-2-aminopropoxy)-1-(difluoromethyl)-6-oxo-1,6-dihydropyridin-3-yl)-2-chloro-8-methyl-8-(trifluoromethyl)-7,8-dihydro-6H-pyrazolo[1,5-a]pyrrolo[2,3-e]pyrimidine-6-carboxamide

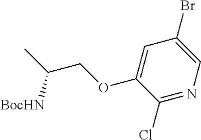 [SMILES: C[C@H](COc1cc(Br)cnc1Cl)NC(=O)OC(C)(C)C]

To a stirred solution of 5-bromo-2-chloropyridin-3-ol (10 g, 47.9 mmol), tert-butyl (R)-(1-hydroxypropan-2-yl)carbamate (16.8 g, 95.9 mmol) and triphenylphosphane (18.9 g, 71.9 mmol) in tetrahydrofuran (100 mL) was added DEAD (12.5 g, 71.9 mmol) dropwise at 0° C. The reaction mixture was stirred at 25° C. for 15 h. The reaction mixture was concentrated under vacuum. The resulting mixture was diluted with water (200 mL), and extracted with ethyl acetate (3×200 mL). The combined organic layers were dried over anhydrous sodium sulfate and concentrated under vacuum. The residue was purified by column chromatography on silica gel using 80% petroleum ether and 20% ethyl acetate as eluents to afford tert-butyl (R)-(1-((5-bromo-2-chloropyridin-3-yl)oxy)propan-2-yl)carbamate (15 g, 85% yield) as a white solid. LC-MS: m/z 365 [M+H]⁺.

### Step 2: tert-butyl (R)-(1-((5-bromo-1-(difluoromethyl)-2-oxo-1,2-dihydropyridin-3-yl)oxy)propan-2-yl)carbamate

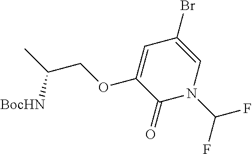 [SMILES: C[C@H](COc1cc(Br)cn(C(F)F)c1=O)NC(=O)OC(C)(C)C]

To a mixture of tert-butyl (R)-(1-((5-bromo-2-chloropyridin-3-yl)oxy)propan-2-yl)carbamate (8.0 g, 21.9 mmol) in acetonitrile (100 mL) was added NaH (1.5 g, 37.2 mmol, 60% in mineral oil) in batches at 0° C. The resulting mixture was stirred at 0° C. for 0.5 h. Then 2,2-difluoro-2-(fluorosulfonyl)acetic acid (6.6 g, 37.2 mmol) was added dropwise. The resulting mixture was stirred at 25° C. for 16 h. The reaction mixture was quenched with water (100 mL). The resulting solution was extracted with ethyl acetate (3×100 mL). The combined organic layers were dried over anhydrous sodium sulfate and concentrated under vacuum. The residue was purified by column chromatography on silica gel using 75% petroleum ether and 25% ethyl acetate as eluents to afford tert-butyl (R)-(1-((5-bromo-1-(difluoromethyl)-2-oxo-1,2-dihydropyridin-3-yl)oxy)propan-2-yl)carbamate (1.1 g, 10% yield) as a yellow oil. LC-MS: m/z 397 [M+H]⁺.

### Step 3: tert-butyl (R)-(1-((1-(difluoromethyl)-5-((diphenylmethylene)amino)-2-oxo-1,2-dihydropyridin-3-yl)oxy)propan-2-yl)carbamate

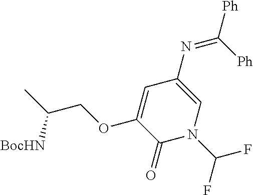 [SMILES: C[C@H](COc1cc(N=C(c2ccccc2)c2ccccc2)cn(C(F)F)c1=O)NC(=O)OC(C)(C)C]

To a mixture of tert-butyl (R)-(1-((5-bromo-1-(difluoromethyl)-2-oxo-1,2-dihydropyridin-3-yl)oxy)propan-2-yl)carbamate (2.1 g, 5.3 mmol) in dioxane (20 mL) were added diphenylmethanimine (1.1 g, 5.8 mmol), Pd₂(dba)₃ (1.6 g, 1.6 mmol), Xantphos (917 mg, 1.6 mmol) and Cs₂CO₃ (5.2 g, 15.8 mmol) under nitrogen atmosphere. The resulting mixture was stirred at 110° C. for 2.5 h. The reaction mixture was cooled to 25° C., and the solid was filtered out. The filtrate was concentrated under vacuum. The residue was purified by column chromatography on silica gel using 75% petroleum ether and 25% ethyl acetate as eluents to afford tert-butyl (R)-(1-((1-(difluoromethyl)-5-((diphenylmethylene)amino)-2-oxo-1,2-dihydropyridin-3-yl)oxy)propan-2-yl)carbamate (840 mg, 32% yield) as a yellow solid. LC-MS: m/z 498 [M+H]⁺.

### Step 4: tert-butyl (R)-(1-((5-amino-1-(difluoromethyl)-2-oxo-1,2-dihydropyridin-3-yl)oxy)propan-2-yl)carbamate

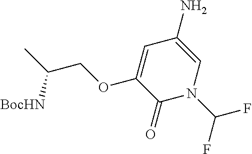 [SMILES: C[C@H](COc1cc(N)cn(C(F)F)c1=O)NC(=O)OC(C)(C)C]

To a stirred solution of tert-butyl (R)-(1-((1-(difluoromethyl)-5-((diphenylmethylene)amino)-2-oxo-1,2-dihydropyridin-3-yl)oxy)propan-2-yl)carbamate (400 mg, 627 μmol) in methanol (5 mL) were added hydroxylamine hydrochloride (87 mg, 1.3 mmol), and sodium acetate (213 mg, 1.6 mmol). The reaction mixture was stirred at 25° C. for 1 h. The reaction mixture was concentrated under vacuum. The residue was diluted with water (20 mL). The resulting solution was extracted with ethyl acetate (3×20 mL). The organic layers were combined, dried over anhydrous sodium sulfate and concentrated under vacuum. The residue was purified by column chromatography on silica gel using 50% petroleum ether and 50% ethyl acetate as eluents to afford tert-butyl (R)-(1-((5-amino-1-(difluoromethyl)-2-oxo-1,2-dihydropyridin-3-yl)oxy)propan-2-yl)carbamate (120 mg, 33% yield) as a yellow solid. LC-MS: m/z 334 [M+H]⁺.

### Step 5: tert-butyl ((R)-1-((5-((R)-2-chloro-8-methyl-8-(trifluoromethyl)-7,8-dihydro-6H-pyrazolo[1,5-a]pyrrolo[2,3-e]pyrimidine-6-carboxamido)-1-(difluoromethyl)-2-oxo-1,2-dihydropyridin-3-yl)oxy)propan-2-yl)carbamate

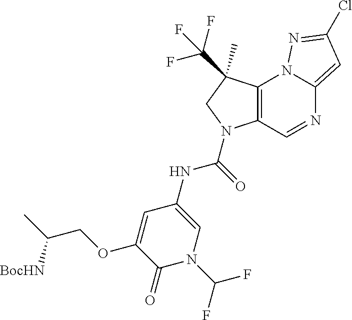 [SMILES: C[C@H](COc1cc(NC(=O)N2C[C@@](C)(C(F)(F)F)c3c2cnc2cc(Cl)nn32)cn(C(F)F)c1=O)NC(=O)OC(C)(C)C]

To a stirred solution of Method M1 isomer 2 (49 mg, 179 μmol) in tetrahydrofuran (1 mL) were added triphosgene (32 mg, 106 μmol) and TEA (36 mg, 356 μmol) at 0° C. The resulting mixture was stirred at 25° C. for 1 h and then filtered. The filtrate was added to a solution of tert-butyl (R)-(1-((5-amino-1-(difluoromethyl)-2-oxo-1,2-dihydropyridin-3-yl)oxy)propan-2-yl)carbamate (50 mg, 98 μmol) in tetrahydrofuran (1 mL). To this solution was then added TEA (36 mg, 299 μmol) and N,N-dimethylpyridin-4-amine (150 mg, 1.5 mmol). The mixture was stirred at 25° C. for 15 h. The reaction mixture was quenched with water (20 mL). The resulting solution was extracted with ethyl acetate (3×20 mL). The combined organic layers were dried over anhydrous sodium sulfate and concentrated under vacuum. The residue was purified by column chromatography on silica gel using 75% petroleum ether and 25% ethyl acetate as eluents to afford tert-butyl ((R)-1-((5-((R)-2-chloro-8-methyl-8-(trifluoromethyl)-7,8-dihydro-6H-pyrazolo[1,5-a]pyrrolo[2,3-e]pyrimidine-6-carboxamido)-1-(difluoromethyl)-2-oxo-1,2-dihydropyridin-3-yl)oxy)propan-2-yl)carbamate (32 mg, 50% yield) as a yellow oil. LC-MS: m/z 636 [M+H]⁺.

### Step 6: (R)—N-(5-((R)-2-aminopropoxy)-1-(difluoromethyl)-6-oxo-1,6-dihydropyridin-3-yl)-2-chloro-8-methyl-8-(trifluoromethyl)-7,8-dihydro-6H-pyrazolo[1,5-a]pyrrolo[2,3-e]pyrimidine-6-carboxamide

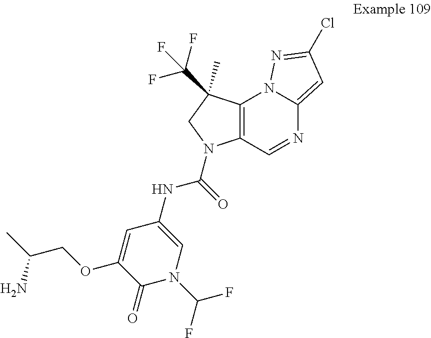 [SMILES: C[C@@H](N)COc1cc(NC(=O)N2C[C@@](C)(C(F)(F)F)c3c2cnc2cc(Cl)nn32)cn(C(F)F)c1=O]

To a stirred solution of tert-butyl ((R)-1-((5-((R)-2-chloro-8-methyl-8-(trifluoromethyl)-7,8-dihydro-6H-pyrazolo[1,5-a]pyrrolo[2,3-e]pyrimidine-6-carboxamido)-1-(difluoromethyl)-2-oxo-1,2-dihydropyridin-3-yl)oxy)propan-2-yl)carbamate (32 mg, 50 μmol) in dichloromethane (3 mL) was added TFA (1 mL). The resulting mixture was stirred at 25° C. for 1 h. The mixture was concentrated under vacuum. The residue was purified by Prep-HPLC and the collected fractions were lyophilized to afford (R)—N-(5-((R)-2-aminopropoxy)-1-(difluoromethyl)-6-oxo-1,6-dihydropyridin-3-yl)-2-chloro-8-methyl-8-(trifluoromethyl)-7,8-dihydro-6H-pyrazolo[1,5-a]pyrrolo[2,3-e]pyrimidine-6-carboxamide (10.2 mg, 60% yield) as a yellow solid. The enantiomer of Example 109 can be prepared analogously using Method M1 isomer 1.

Example 109: ¹H NMR (400 MHz, DMSO-d₆) δ: 9.32 (s, 1H), 8.85 (br, 1H), 7.95 (t, J=59.8 Hz, 1H), 7.71 (d, J=2.4 Hz, 1H), 7.11 (d, J=2.4 Hz, 1H), 7.04 (s, 1H), 4.71 (d, J=11.2 Hz, 1H), 4.20 (d, J=11.6 Hz, 1H), 3.62-3.72 (m, 2H), 3.15-3.21 (m, 3H), 1.97 (s, 3H), 1.07 (d, J=6.4 Hz, 3H). LC-MS: m/z 536 [M+H]⁺.

### Method Z3

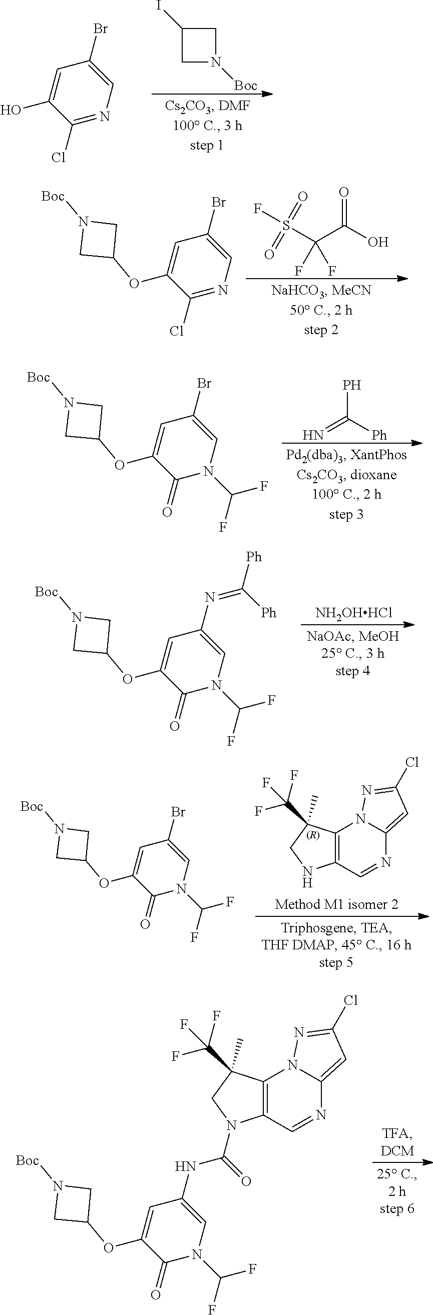

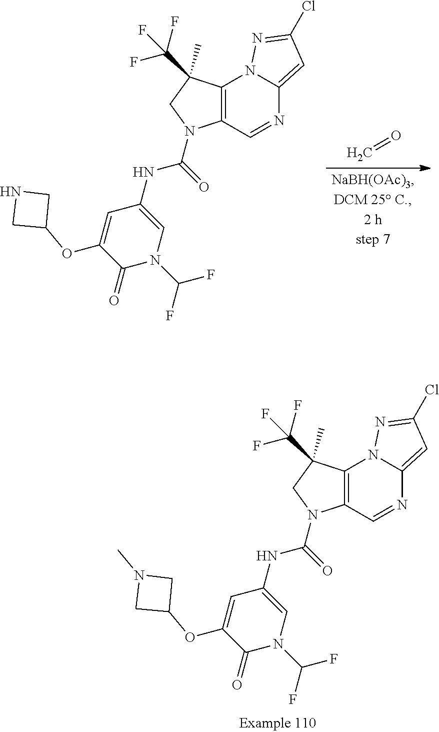

### Example 110: (R)-2-chloro-N-(1-(difluoromethyl)-5-((1-methylazetidin-3-yl)oxy)-6-oxo-1,6-dihydropyridin-3-yl)-8-methyl-8-(trifluoromethyl)-7,8-dihydro-6H-pyrazolo[1,5-a]pyrrolo[2,3-e]pyrimidine-6-carboxamide

### Step 1: tert-butyl 3-((5-bromo-2-chloropyridin-3-yl)oxy)azetidine-1-carboxylate

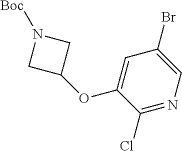 [SMILES: CC(C)(C)OC(=O)N1CC(Oc2cc(Br)cnc2Cl)C1]

To a stirred solution of 5-bromo-2-chloropyridin-3-ol (5.0 g, 24.0 mmol) and tert-butyl 3-iodoazetidine-1-carboxylate (6.8 g, 24.0 mmol) in DMF (50 mL) was added Cs₂CO₃ (15.6 g, 48.0 mmol). The resulting mixture was stirred at 100° C. for 3 h. The reaction was cooled to 25° C. The reaction mixture was quenched with water (150 mL). The resulting solution was extracted with ethyl acetate (3×250 mL). The combined organic layers were dried over anhydrous sodium sulfate and concentrated under vacuum. The residue was purified by column chromatography on silica gel using 60% petroleum ether and 40% ethyl acetate as eluents to afford tert-butyl 3-((5-bromo-2-chloropyridin-3-yl)oxy)azetidine-1-carboxylate (8.0 g, 90% yield) as a white solid. ¹HNMR (400 MHz, Chloroform-d) δ: 8.11 (d, J=2.0 Hz, 1H), 6.98 (d, J=2.0 Hz, 1H), 4.90-4.92 (m, 1H), 4.35-4.39 (m, 2H), 4.09-4.11 (m, 2H), 1.46 (s, 9H). LC-MS: m/z 363 [M+H]⁺.

### Step 2: tert-butyl 3-((5-bromo-1-(difluoromethyl)-2-oxo-1,2-dihydropyridin-3-yl)oxy)azetidine-1-carboxylate

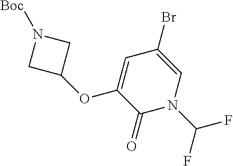 [SMILES: CC(C)(C)OC(=O)N1CC(Oc2cc(Br)cn(C(F)F)c2=O)C1]

To a solution of tert-butyl 3-((5-bromo-2-chloropyridin-3-yl)oxy)azetidine-1-carboxylate (3.0 g, 8.2 mmol) in acetonitrile (30 mL) were added 2,2-difluoro-2-(fluorosulfonyl)acetic acid (4.4 g, 24.7 mmol) and NaHCO₃ (1.5 g, 8.6 mmol). The resulting mixture was stirred at 50° C. for 2 h. The reaction mixture was cooled to 25° C. The reaction mixture was quenched by the addition of water (150 mL). The resulting solution was extracted with ethyl acetate (3×150 mL). The combined organic layers were dried over anhydrous sodium sulfate and concentrated under vacuum. The residue was purified by column chromatography on silica gel using 50% petroleum ether and 50% ethyl acetate as eluents to afford tert-butyl 3-((5-bromo-1-(difluoromethyl)-2-oxo-1,2-dihydropyridin-3-yl)oxy)azetidine-1-carboxylate (1.0 g, 54% yield) as a white solid. ¹H NMR (400 MHz, Chloroform-d) δ: 8.10 (d, J=2.0 Hz, 1H), 7.03 (d, J=2.0 Hz, 1H), 5.06-5.08 (m, 1H), 4.36 (s, 1H), 4.09 (d, J=8.2 Hz, 2H), 3.98 (t, J=7.8 Hz, 2H), 1.46 (s, 9H). LC-MS: m/z 395 [M+H]⁺.

### Step 3: tert-butyl 3-((1-(difluoromethyl)-5-((diphenylmethylene)amino)-2-oxo-1,2-dihydropyridin-3-yl)oxy)azetidine-1-carboxylate

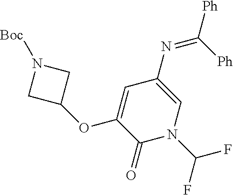 [SMILES: CC(C)(C)OC(=O)N1CC(Oc2cc(N=C(c3ccccc3)c3ccccc3)cn(C(F)F)c2=O)C1]

To a stirred solution of tert-butyl 3-((5-bromo-1-(difluoromethyl)-2-oxo-1,2-dihydropyridin-3-yl)oxy)azetidine-1-carboxylate (600 mg, 1.5 mmol) and diphenylmethanimine (550 mg, 3.3 mmol) in dioxane (10 mL) were added XantPhos (175 mg, 302.8 mmol), Pd₂(dba)₃ (157 mg, 302.8 μmol) and Cs₂CO₃ (975 mg, 3.0 mmol) under nitrogen atmosphere. The resulting mixture was stirred at 110° C. for 2 h. The reaction mixture was cooled to 25° C. The solid was filtered out. The filtrate was concentrated under vacuum. The residue was purified by column chromatography on silica gel using 70% petroleum ether and 30% ethyl acetate as eluents to afford tert-butyl 3-((1-(difluoromethyl)-5-((diphenylmethylene)amino)-2-oxo-1,2-dihydropyridin-3-yl)oxy)azetidine-1-carboxylate (400 mg, 37% yield) as a white solid. ¹H NMR (400 MHz, Chloroform-d) δ: 8.10-8.90 (m, 11H), 7.71 (t, J=60.3 Hz, 1H), 5.06-5.08 (m, 1H), 4.36 (s, 1H), 4.09 (d, J=8.2 Hz, 2H), 3.98 (t, J=7.8 Hz, 2H), 1.46 (s, 9H). LC-MS: m/z 496 [M+H]⁺.

### Step 4: tert-butyl 3-((5-amino-1-(difluoromethyl)-2-oxo-1,2-dihydropyridin-3-yl)oxy)azetidine-1-carboxylate

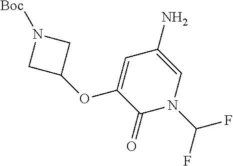 [SMILES: CC(C)(C)OC(=O)N1CC(Oc2cc(N)cn(C(F)F)c2=O)C1]

Into a 50 mL round bottom flask were placed tert-butyl 3-((1-(difluoromethyl)-5-((diphenylmethylene)amino)-2-oxo-1,2-dihydropyridin-3-yl)oxy)azetidine-1-carboxylate (400 mg, 807.8 μmol), hydroxylamine hydrochloride (112 mg, 1.6 mmol), sodium acetate (283 mg, 3.4 mmol) and methanol (10 mL). The mixture was stirred at 25° C. for 3 h. The mixture was concentrated under vacuum. The residue was purified by column chromatography on silica gel using 50% petroleum ether and 50% ethyl acetate as eluents to afford tert-butyl 3-((5-amino-1-(difluoromethyl)-2-oxo-1,2-dihydropyridin-3-yl)oxy)azetidine-1-carboxylate (150 mg, 30% yield) as a yellow solid. ¹H NMR (400 MHz, Chloroform-d) δ: 7.71 (t, J=60.3 Hz, 1H), 6.58 (d, J=2.4 Hz, 1H), 6.10 (d, J=2.4 Hz, 1H), 4.22-4.34 (m, 2H), 4.80-4.82 (m, 1H), 4.09-4.17 (m, 2H), 1.44 (s, 9H). LC-MS: m/z 332 [M+H]⁺.

### Step 5: tert-butyl (R)-3-((5-(2-chloro-8-methyl-8-(trifluoromethyl)-7,8-dihydro-6H-pyrazolo[1,5-a]pyrrolo[2,3-e]pyrimidine-6-carboxamido)-1-(difluoromethyl)-2-oxo-1,2-dihydropyridin-3-yl)oxy)azetidine-1-carboxylate

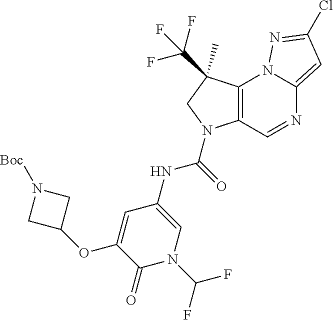 [SMILES: CC(C)(C)OC(=O)N1CC(Oc2cc(NC(=O)N3C[C@@](C)(C(F)(F)F)c4c3cnc3cc(Cl)nn43)cn(C(F)F)c2=O)C1]

To a stirred solution of Method M1 isomer 2 (100 mg, 362.3 μmol) in tetrahydrofuran (4 mL) were added triphosgene (64 mg, 217.4 μmol) and TEA (55 mg, 543.4 μmol) at 0° C. The resulting mixture was stirred at 25° C. for 0.5 h and then filtered. The filtrate was added to a solution of tert-butyl 3-((5-amino-1-(difluoromethyl)-2-oxo-1,2-dihydropyridin-3-yl)oxy)azetidine-1-carboxylate (120 mg, 362.3 μmol) in tetrahydrofuran (2 mL). To this solution was then added N,N-dimethylpyridin-4-amine (53 mg, 434.8 μmol) and TEA (363 mg, 3.6 mmol). The mixture was stirred at 40° C. for 2 h. The mixture was poured into water (30 mL) and extracted with ethyl acetate (3×30 mL). The combined organic layers were washed with brine (50 mL), dried over anhydrous sodium sulfate and concentrated under vacuum. The residue was purified by Prep-TLC using 95% dichloromethane and 5% methanol as eluents to afford tert-butyl (R)-3-((5-(2-chloro-8-methyl-8-(trifluoromethyl)-7,8-dihydro-6H-pyrazolo[1,5-a]pyrrolo[2,3-e]pyrimidine-6-carboxamido)-1-(difluoromethyl)-2-oxo-1,2-dihydropyridin-3-yl)oxy)azetidine-1-carboxylate (80 mg, 30% yield) as a white solid. LC-MS: m/z 634 [M+H]⁺.

### Step 6: (R)—N-(5-(azetidin-3-yloxy)-1-(difluoromethyl)-6-oxo-1,6-dihydropyridin-3-yl)-2-chloro-8-methyl-8-(trifluoromethyl)-7,8-dihydro-6H-pyrazolo[1,5-a]pyrrolo[2,3-e]pyrimidine-6-carboxamide

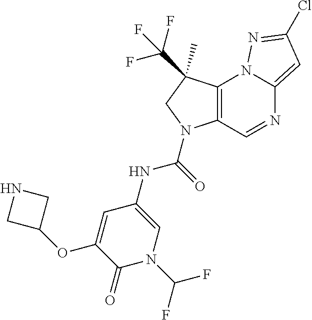 [SMILES: C[C@@]1(C(F)(F)F)CN(C(=O)Nc2cc(OC3CNC3)c(=O)n(C(F)F)c2)c2cnc3cc(Cl)nn3c21]

To a stirred solution of tert-butyl (R)-3-((5-(2-chloro-8-methyl-8-(trifluoromethyl)-7,8-dihydro-6H-pyrazolo[1,5-a]pyrrolo[2,3-e]pyrimidine-6-carboxamido)-1-(difluoromethyl)-2-oxo-1,2-dihydropyridin-3-yl)oxy)azetidine-1-carboxylate (80 mg, 126.3 μmol) in dichloromethane (10 mL) was added TFA (2 mL). The mixture was stirred at 25° C. for 2 h. The resulting mixture was concentrated under vacuum. The residue was added saturated aqueous NaHCO₃ solution (40 mL). The resulting solution was extracted with ethyl acetate (3×40 mL). The combined organic layers were washed with brine (50 mL) and dried over anhydrous sodium sulfate and concentrated under vacuum. The residue was purified by column chromatography on silica gel using 30% petroleum ether and 70% ethyl acetate as eluents to afford (R)—N-(5-(azetidin-3-yloxy)-1-(difluoromethyl)-6-oxo-1,6-dihydropyridin-3-yl)-2-chloro-8-methyl-8-(trifluoromethyl)-7,8-dihydro-6H-pyrazolo[1,5-a]pyrrolo[2,3-e]pyrimidine-6-carboxamide (50 mg, 76% yield) as a yellow solid. LC-MS: m/z 534 [M+H]⁺.

### Step 6: (R)-2-chloro-N-(1-(difluoromethyl)-5-((1-methylazetidin-3-yl)oxy)-6-oxo-1,6-dihydropyridin-3-yl)-8-methyl-8-(trifluoromethyl)-7,8-dihydro-6H-pyrazolo[1,5-a]pyrrolo[2,3-e]pyrimidine-6-carboxamide

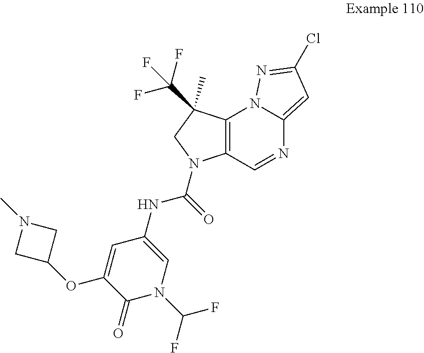 [SMILES: CN1CC(Oc2cc(NC(=O)N3C[C@@](C)(C(F)(F)F)c4c3cnc3cc(Cl)nn43)cn(C(F)F)c2=O)C1]

To a stirred solution of (R)—N-(5-(azetidin-3-yloxy)-1-(difluoromethyl)-6-oxo-1,6-dihydropyridin-3-yl)-2-chloro-8-methyl-8-(trifluoromethyl)-7,8-dihydro-6H-pyrazolo[1,5-a]pyrrolo[2,3-e]pyrimidine-6-carboxamide (50 mg, 93.8 μmol) in dichloromethane (5 mL) was added formaldehyde (0.1 mL, 469 μmol, 40% in water) and sodium triacetoxyborohydride (29 mg, 140.7 μmol). The mixture was stirred at 25° C. for 2 h. The mixture was concentrated under vacuum. The residue was purified by Prep-HPLC purification and the collected fractions were lyophilized to give (R)-2-chloro-N-(1-(difluoromethyl)-5-((1-methylazetidin-3-yl)oxy)-6-oxo-1,6-dihydropyridin-3-yl)-8-methyl-8-(trifluoromethyl)-7,8-dihydro-6H-pyrazolo[1,5-a]pyrrolo[2,3-e]pyrimidine-6-carboxamide (9 mg, 28% yield) as a white solid. The enantiomer of Example 110 can be prepared analogously using Method M1 isomer 1.

Example 110: ¹H NMR (300 MHz, Methanol-d₄) δ: 9.32 (s, 1H), 7.86 (t, J=60.0 Hz, 1H), 7.66 (s, 1H), 7.12 (s, 1H), 6.78 (s, 1H), 5.03-5.05 (m, 1H), 4.67 (d, J=10.8 Hz, 1H), 4.43-4.53 (m, 2H), 4.10-4.15 (m, 3H), 2.90 (s, 3H), 2.03 (s, 3H). LC-MS: m/z 548 [M+H]⁺.

### Method A4

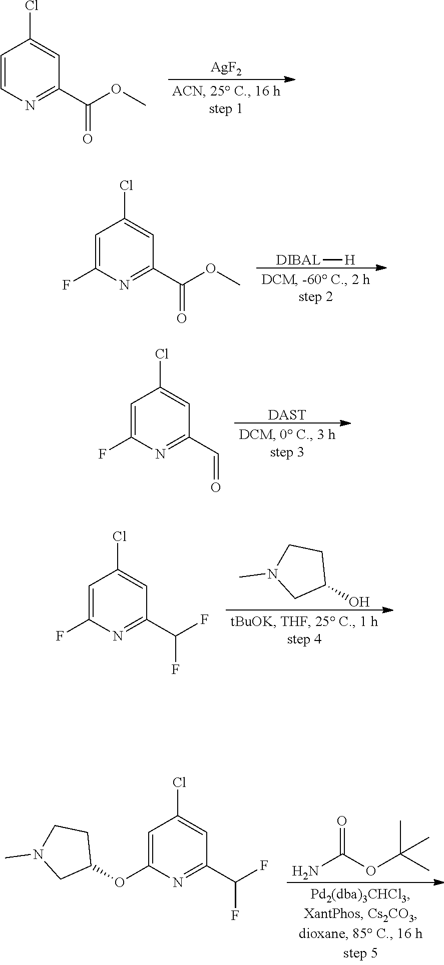

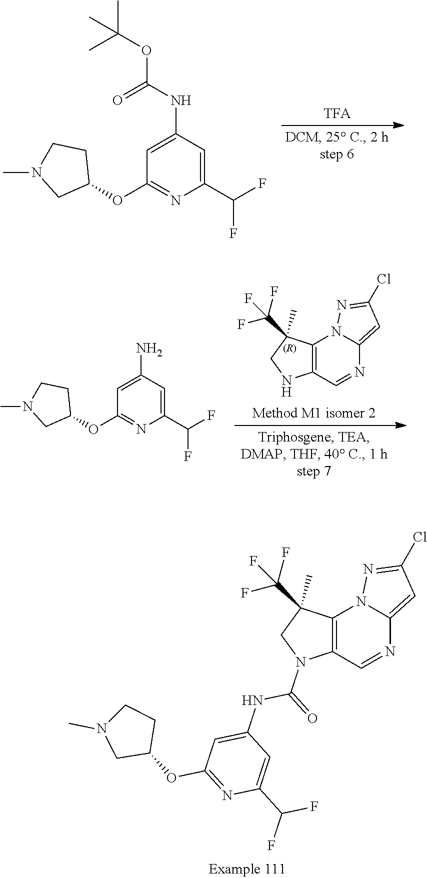

### Examples 111: (R)-2-chloro-N-(2-(difluoromethyl)-6-(((S)-1-methylpyrrolidin-3-yl)oxy)pyridin-4-yl)-8-methyl-8-(trifluoromethyl)-7,8-dihydro-6H-pyrazolo[1,5-a]pyrrolo[2,3-e]pyrimidine-6-carboxamide

### Step 1: methyl 4-chloro-6-fluoropicolinate

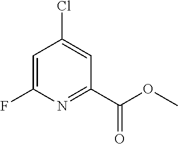

To a stirred solution of methyl 4-chloropicolinate (40 g, 233.9 mmol) in acetonitrile (1200 mL) was added AgF₂ (101.7 g, 701.7 mmol) under nitrogen atmosphere. The resulting mixture was stirred at 25° C. for 16 h. The reaction mixture was filtered and the collected solid was washed with ethyl acetate (3×200 mL). The resulting solution was concentrated under vacuum. The residue was purified by column chromatography on silica gel using 75% petroleum ether and 25% ethyl acetate as eluents to afford methyl 4-chloro-6-fluoropicolinate (11.8 g, 26% yield) as a white solid. ¹H NMR (300 MHz, DMSO-d₆) δ: 8.01-8.08 (m, 1H), 7.20-7.28 (m, 1H), 4.03 (s, 3H). LC-MS: m/z 190 [M+H]⁺.

### Step 2: 4-chloro-6-fluoropicolinaldehyde

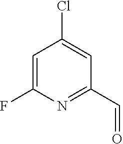

To a solution of methyl 4-chloro-6-fluoropicolinate (8.0 g, 42.3 mmol) in dichloromethane (200 mL) was added diisobutylaluminium hydride (80 mL, 80 mmol, 1M in dichloromethane) at −60° C. under nitrogen. The resulting mixture was stirred at −60° C. for 2 h. The reaction mixture was quenched with saturated aqueous potassium sodium tartrate tetrahydrate solution (200 mL). The solid was filtrated and washed with dichloromethane (3×100 mL). The resulting solution was extracted with dichloromethane (3×200 mL). The combined organic layers were dried over anhydrous sodium sulfate and concentrated under vacuum. The residue was purified by column chromatography on silica gel using 75% petroleum ether and 25% ethyl acetate as eluents to afford 4-chloro-6-fluoropicolinaldehyde (4.9 g, 72% yield) as a yellow solid. ¹H NMR (400 MHz, Chloroform-d) δ: 9.92 (s, 1H), 7.84-7.85 (m, 1H), 7.22-7.23 (m, 1H). LC-MS: m/z 160 [M+H]⁺.

### Step 3: 4-chloro-2-(difluoromethyl)-6-fluoropyridine

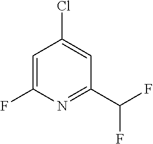 [SMILES: Fc1cc(Cl)cc(C(F)F)n1]

To a stirred solution of 4-chloro-6-fluoropicolinaldehyde (4.9 g, 30.8 mmol) in dichloromethane (163 mL) was added DAST (14.9 g, 92.4 mmol) at −30° C. The reaction mixture was stirred at 0° C. for 2 h. The reaction mixture was quenched with water (200 mL). The resulting solution was extracted with dichloromethane (3×200 mL). The combined organic layers were washed by saturated aqueous NaHCO₃ solution (200 mL) and dried over anhydrous sodium sulfate. The resulting solution was concentrated under vacuum to give 4-chloro-2-(difluoromethyl)-6-fluoropyridine (4 g, 71% yield) as a yellow oil which was used without further purification. ¹H NMR (400 MHz, Chloroform-d) δ: 7.59 (d, J=28 Hz, 1H), 7.09-7.11 (m, 1H), 6.52 (t, J=56 Hz, 1H).

### Step 4: (S)-4-chloro-2-(difluoromethyl)-6-((1-methylpyrrolidin-3-yl)oxy)pyridine

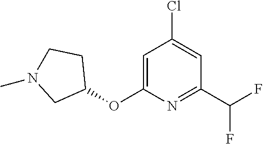

To a stirred solution of 4-chloro-2-(difluoromethyl)-6-fluoropyridine (3.0 g, 16.3 mmol) in tetrahydrofuran (90 mL) were added (S)-1-methylpyrrolidin-3-ol (1.5 g, 14.9 mmol) and t-BuOK (3.3 g, 29.8 mmol). The resulting mixture was stirred at 0° C. for 1 h. The reaction mixture was quenched with water (50 mL). The resulting solution was extracted with ethyl acetate (3×200 mL). The combined organic layers were dried over anhydrous sodium sulfate and concentrated under vacuum. The residue was purified by column chromatography on silica gel using 10% methanol and 90% dichloromethane as eluents to afford (S)-4-chloro-2-(difluoromethyl)-6-((1-methylpyrrolidin-3-yl)oxy)pyridine (1.5 g, 71% yield) as a yellow oil. LC-MS: m/z 263 [M+H]⁺.

### Step 5: tert-butyl (S)-(2-(difluoromethyl)-6-((1-methylpyrrolidin-3-yl)oxy)pyridin-4-yl)carbamate

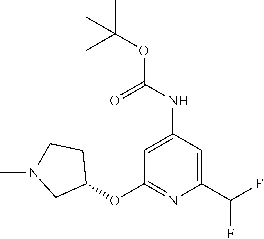 [SMILES: CN1CC[C@H](Oc2cc(NC(=O)OC(C)(C)C)cc(C(F)F)n2)C1]

To a stirred solution of (S)-4-chloro-2-(difluoromethyl)-6-((1-methylpyrrolidin-3-yl)oxy)pyridine (500 mg, 1.9 mmol) in dioxane (15 mL) were added tert-butyl carbamate (664 mg, 5.7 mmol), Pd₂(dba)₃CHCl₃ (198 mg, 0.2 mmol), XantPhos (232 mg, 0.4 mmol) and Cs₂CO₃ (1.3 g, 3.8 mmol) under nitrogen atmosphere. The resulting mixture was stirred at 85° C. for 16 h. The mixture was allowed to cool down to 25° C. The mixture was concentrated under reduced pressure. The residue was purified by column chromatography on silica gel using 10% methanol and 90% dichloromethane as eluents to afford tert-butyl (S)-(2-(difluoromethyl)-6-((1-methylpyrrolidin-3-yl)oxy)pyridin-4-yl)carbamate (270 mg, 41% yield) as a yellow solid. LC-MS: m/z 344 [M+H]⁺.

### Step 6: (S)-2-(difluoromethyl)-6-((1-methylpyrrolidin-3-yl)oxy)pyridin-4-amine

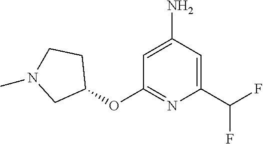 [SMILES: CN1CC[C@H](Oc2cc(N)cc(C(F)F)n2)C1]

To a solution of tert-butyl (S)-(2-(difluoromethyl)-6-((1-methylpyrrolidin-3-yl)oxy)pyridin-4-yl)carbamate (270 mg, 787 μmol) in dichloromethane (10 mL) was added TFA (2 mL). The resulting mixture was stirred at 25° C. for 2 h. The mixture was concentrated under vacuum. The pH was adjusted to 8 with saturated aqueous NaHCO₃ solution. The resulting mixture was extracted with ethyl acetate (3×50 mL). The combined organic layers were dried over anhydrous sodium sulfate and concentrated under vacuum. The residue was purified by column chromatography on silica gel using 10% methanol and 90% dichloromethane as eluents to afford (S)-2-(difluoromethyl)-6-((1-methylpyrrolidin-3-yl)oxy)pyridin-4-amine (170 mg, 81% yield) as a yellow oil. LC-MS: m/z 244 [M+H]⁺.

### Step 7: (R)-2-chloro-N-(2-(difluoromethyl)-6-(((S)-1-methylpyrrolidin-3-yl)oxy)pyridin-4-yl)-8-methyl-8-(trifluoromethyl)-7,8-dihydro-6H-pyrazolo[1,5-a]pyrrolo[2,3-e]pyrimidine-6-carboxamide

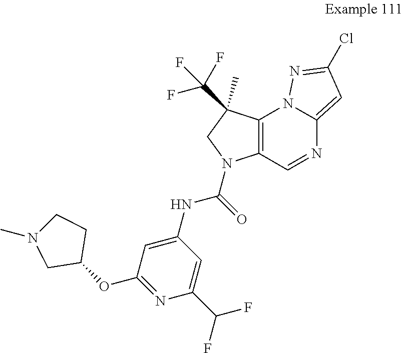 [SMILES: CN1CC[C@H](Oc2cc(NC(=O)N3C[C@@](C)(C(F)(F)F)c4c3cnc3cc(Cl)nn43)cc(C(F)F)n2)C1]

To a stirred solution of (S)-2-(difluoromethyl)-6-((1-methylpyrrolidin-3-yl)oxy)pyridin-4-amine (62 mg, 255.1 μmol) in tetrahydrofuran (10 mL) were added triphosgene (23 mg, 77.7 μmol) and TEA (20 mg, 190 μmol) at 0° C. The resulting mixture was stirred at 28° C. for 0.5 h and then filtered. The filtrate was added to a solution of Method M1 isomer 2 (35 mg, 127 μmol) in tetrahydrofuran (1 mL). To this solution was then added TEA (110 mg, 1.1 mmol) and N,N-dimethylpyridin-4-amine (27 mg, 217.4 μmol). The mixture was stirred at 40° C. for 1 h. The reaction mixture was quenched with water (50 mL). The resulting solution was extracted with ethyl acetate (3×50 mL). The combined organic layers were dried over anhydrous sodium sulfate and concentrated under vacuum. The residue was purified by column chromatography on silica gel using 10% methanol and 90% dichloromethane as eluents to afford crude product. The crude product was purified by Prep-HPLC and the collected fractions were lyophilized to give (R)-2-chloro-N-(2-(difluoromethyl)-6-(((S)-1-methylpyrrolidin-3-yl)oxy)pyridin-4-yl)-8-methyl-8-(trifluoromethyl)-7,8-dihydro-6H-pyrazolo[1,5-a]pyrrolo[2,3-e]pyridine-6-carboxamide (15.8 mg, 33% yield) as a white solid. The enantiomer of Example 111 can be prepared analogously using Method M1 isomer 1.

Example 111: ¹H NMR (400 MHz, DMSO-d₆) δ: 9.52 (br, 1H), 9.32 (s, 1H), 7.48 (d, J=1.2 Hz, 1H), 7.23 (s, 1H), 7.07 (s, 1H), 6.81 (t, J=54 Hz, 1H), 5.33 (s, 1H), 4.84-4.87 (m, 1H), 4.25-4.28 (m, 1H), 2.78-2.82 ((m, 1H), 2.60-2.68 (m, 2H), 2.29-2.40 (m, 5H), 2.27 (s, 3H), 1.90-1.96 (m, 1H). LC-MS: m/z 546 [M+H]⁺.

### Method B4

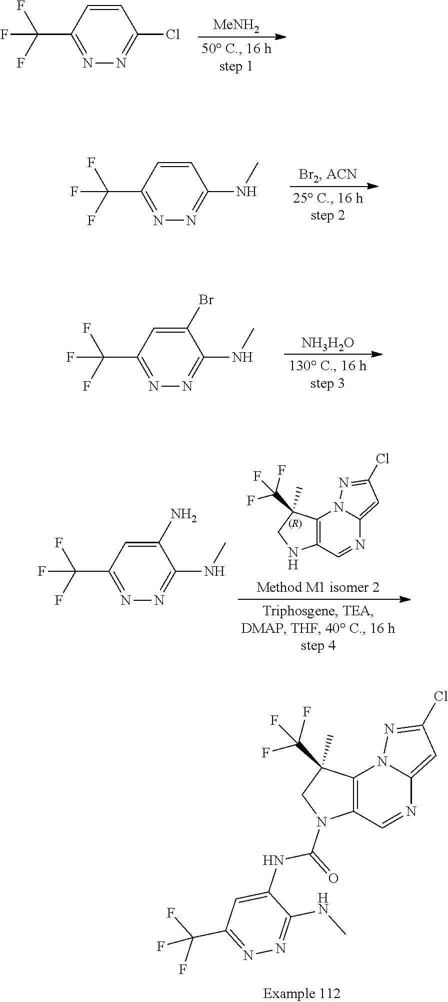

### Example 112: (R)-2-chloro-8-methyl-N-(3-(methylamino)-6-(trifluoromethyl)pyridazin-4-yl)-8-(trifluoromethyl)-7,8-dihydro-6H-pyrazolo[1,5-a]pyrrolo[2,3-e]pyrimidine-6-carboxamide

### Step 1: N-methyl-6-(trifluoromethyl)pyridazin-3-amine

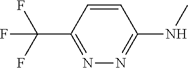

A solution of 3-chloro-6-(trifluoromethyl)pyridazine (5.0 g, 27.4 mmol) in methanamine (50 mL, 100 mmol, 2 M in THF) was stirred at 50° C. for 16 h. The reaction mixture was concentrated under vacuum. The residue was purified by column chromatography on silica gel using 50% petroleum ether and 50% ethyl acetate to afford N-methyl-6-(trifluoromethyl)pyridazin-3-amine (2.5 g, 51% yield) as a white solid. ¹H NMR (300 MHz, DMSO-d₆) δ: 7.64 (d, J=9.6 Hz, 1H), 7.57 (br, 1H), 6.93 (d, J=9.6 Hz, 1H), 2.93 (d, J=4.8 Hz, 3H). LC-MS: m/z 178 [M+H]⁺.

### Step 2: 4-bromo-N-methyl-6-(trifluoromethyl)pyridazin-3-amine

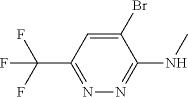

To a stirred solution of N-methyl-6-(trifluoromethyl)pyridazin-3-amine (2.7 g, 15.2 mmol) in acetonitrile (50 mL) was added bromine (4.9 g, 30.5 mmol) dropwise. The reaction mixture was stirred at 25° C. for 16 h. The reaction mixture was quenched with saturated aqueous NaHCO₃ solution (50 mL). The resulting mixture was extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate as eluents to afford 4-bromo-N-methyl-6-(trifluoromethyl) pyridazin-3-amine (1.4 g, 32% yield) as a yellow solid. ¹H NMR (300 MHz, DMSO-d₆) δ: 8.24 (s, 1H), 7.45 (br, 1H), 3.02 (d, J=4.8 Hz, 3H). LC-MS: m/z 256 [M+H]⁺.

### Step 3: N-methyl-6-(trifluoromethyl) pyridazine-3,4-diamine

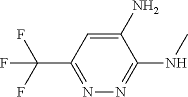

A mixture of 4-bromo-N-methyl-6-(trifluoromethyl) pyridazin-3-amine (500 mg, 1.9 mmol) in ammonia (15 mL) was stirred at 130° C. for 16 h. The resulting mixture was extracted with ethyl acetate (3×10 mL). The combined organic layers were washed with brine (20 mL), dried over anhydrous sodium sulfate and concentrated under vacuum. The residue was purified by Prep-HPLC and the collected fractions were lyophilized to afford N-methyl-6-(trifluoromethyl) pyridazine-3,4-diamine (300 mg, 80% yield) as a white solid. ¹H NMR (400 MHz, DMSO-d₆) S 6.65 (s, 1H), 6.49 (d, J=4.8 Hz, 1H), 6.26 (s, 2H), 2.95 (d, J=4.8 Hz, 3H). LC-MS: m/z 193 [M+H]⁺.

### Step 4: (R)-2-chloro-8-methyl-N-(3-(methylamino)-6-(trifluoromethyl) pyridazin-4-yl)-8-(trifluoromethyl)-7,8-dihydro-6H-pyrazolo[1,5-a]pyrrolo[2,3-e]pyrimidine-6-carboxamide

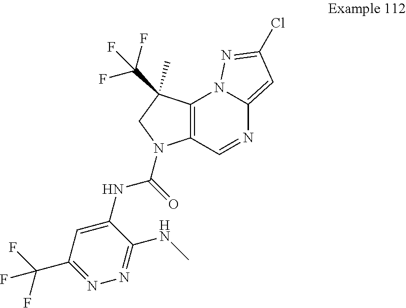 [SMILES: CNc1nnc(C(F)(F)F)cc1NC(=O)N1C[C@@](C)(C(F)(F)F)c2c1cnc1cc(Cl)nn21]

To a stirred solution of Method M1 isomer 2 (100 mg, 361 μmol) in tetrahydrofuran (3 mL) were added triphosgene (32 mg, 108 μmol) and TEA (55 mg, 542 μmol). The resulting mixture was stirred at 25° C. for 1 h and then filtered. The filtrate was added to a solution of N-methyl-6-(trifluoromethyl)pyridazine-3,4-diamine (104 mg, 542 μmol) in tetrahydrofuran (3 mL). To this solution were added N,N-dimethylpyridin-4-amine (88 mg, 723 μmol) and TEA (366 mg, 3.6 mmol). The mixture was stirred at 40° C. for 16 h. The mixture was concentrated under vacuum. The residue was purified by Prep-HPLC and the collected fractions were lyophilized to afford (R)-2-chloro-8-methyl-N-(3-(methylamino)-6-(trifluoromethyl)pyridazin-4-yl)-8-(trifluoromethyl)-7,8-dihydro-6H-pyrazolo[1,5-a]pyrrolo[2,3-e]pyrimidine-6-carboxamide (2 mg, 1% yield) as a yellow solid. The enantiomer of Example 112 can be prepared analogously using Method M1 isomer 1.

Example 112: ¹H NMR (300 MHz, DMSO-d₆) δ: 9.38 (s, 1H), 8.77 (br, 1H), 7.96 (s, 1H), 7.25 (s, 1H), 7.03 (s, 1H), 4.84 (d, J=12.0 Hz, 1H), 4.25 (d, J=12.0 Hz, 1H), 3.06 (d, J=4.8 Hz, 3H), 1.96 (s, 3H). LC-MS: m/z 495 [M+H]⁺.

### Method C4

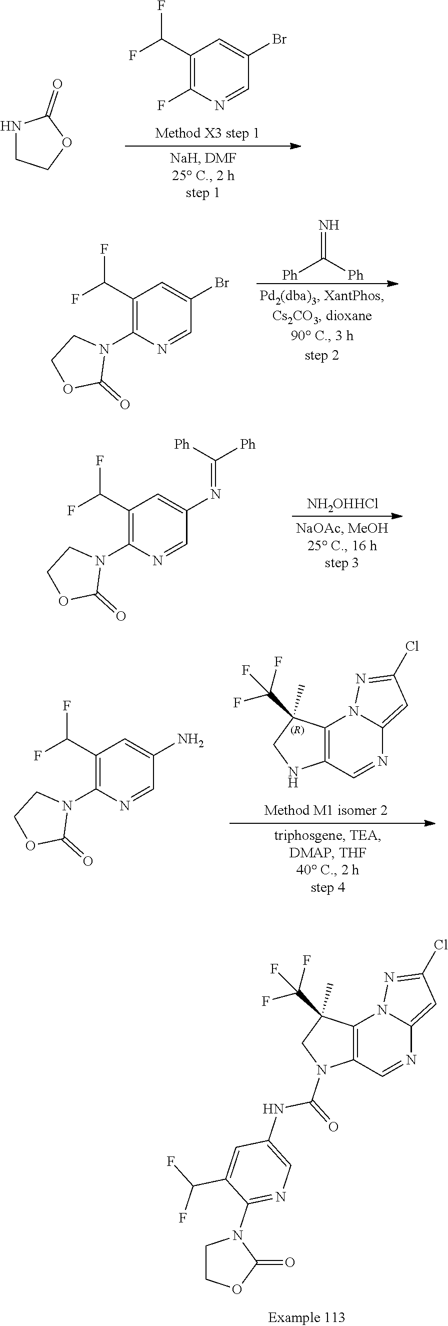

### Example 113: (R)-2-chloro-N-(5-(difluoromethyl)-6-(2-oxooxazolidin-3-yl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 3-(5-bromo-3-(difluoromethyl)pyridin-2-yl)oxazolidin-2-one

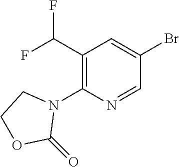 [SMILES: O=C1OCCN1c1ncc(Br)cc1C(F)F]

To a stirred solution of oxazolidin-2-one (539 mg, 6.2 mmol) in N,N-Dimethylformamide (5 mL) was added NaH (283 mg, 7.1 mmol, 60% in mineral oil) in batches at 0° C. The reaction mixture was stirred at 0° C. for 0.5 h. Then 5-bromo-3-(difluoromethyl)-2-fluoro-pyridine (Method X3 step 1; 2.0 g, 8.8 mmol) was added into the mixture. The resulting mixture was stirred at 25° C. for 2 h. The reaction mixture was quenched with water (100 mL). The resulting solution was extracted with ethyl acetate (3×100 mL). The combined organic layers were dried over anhydrous sodium sulfate and concentrated under vacuum. The residue was purified by column chromatography on silica gel using 50% petroleum ether and 50% ethyl acetate as eluents to afford 3-(5-bromo-3-(difluoromethyl)pyridin-2-yl)oxazolidin-2-one (800 mg, 31% yield) as a colorless oil. ¹H NMR (300 MHz, Chloroform-d) δ: 8.55-8.56 (m, 1H), 8.15-8.16 (m, 1H), 7.15 (t, J=55.8 Hz, 1H), 4.54-4.65 (m, 2H), 4.26-4.31 (m, 2H). LC-MS: m/z 293 [M+H]⁺.

### Step 2: 3-(3-(difluoromethyl)-5-((diphenylmethylene)amino)pyridin-2-yl)oxazolidin-2-one

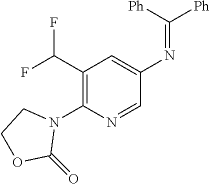 [SMILES: O=C1OCCN1c1ncc(N=C(c2ccccc2)c2ccccc2)cc1C(F)F]

To a mixture of 3-(5-bromo-3-(difluoromethyl)pyridin-2-yl)oxazolidin-2-one (400 mg, 1.4 mmol) in 1,4-dioxane (8 mL) were added XantPhos (59 mg, 102.4 μmol), Pd₂(dba)₃ (62 mg, 68.2 μmol), Cs₂CO₃ (1.1 g, 3.4 mmol) and diphenylmethanimine (247 mg, 1.4 mmol). The resulting mixture was stirred at 90° C. for 3 h under nitrogen atmosphere. After cooled to 25° C., the reaction mixture was quenched with water (50 mL). The resulting solution was extracted with ethyl acetate (3×50 mL). The combined organic layers were dried over anhydrous sodium sulfate and concentrated under vacuum. The residue was purified by column chromatography on silica gel using 60% petroleum ether and 40% ethyl acetate as eluents to afford 3-(3-(difluoromethyl)-5-((diphenylmethylene)amino)pyridin-2-yl)oxazolidin-2-one (500 mg, 93% yield) as a white solid. LC-MS: m/z 394 [M+H]⁺.

### Step 3: 3-(5-amino-3-(difluoromethyl)pyridin-2-yl)oxazolidin-2-one

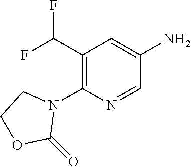 [SMILES: Nc1cnc(N2CCOC2=O)c(C(F)F)c1]

To a stirred solution of 3-(3-(difluoromethyl)-5-((diphenylmethylene)amino)pyridin-2-yl)oxazolidin-2-one (400 mg, 1.2 mmol) and sodium acetate (346 mg, 2.5 mmol) in methanol (8 mL) was added hydroxylamine hydrochloride (141 mg, 2.1 mmol). The resulting mixture was stirred at 25° C. for 16 h. The reaction mixture was quenched with water (50 mL). The resulting solution was extracted with ethyl acetate (3×50 mL). The combined organic layers were dried over anhydrous sodium sulfate and concentrated under vacuum. The residue was purified by column chromatography on silica gel using 40% petroleum ether and 60% ethyl acetate as eluents to afford 3-(5-amino-3-(difluoromethyl)pyridin-2-yl)oxazolidin-2-one (180 mg, 77% yield) as a yellow solid. ¹H NMR (300 MHz, Chloroform-d) δ: 7.96-7.98 (m, 1H), 7.27-7.28 (m, 1H), 7.00 (t, J=55.5 Hz, 1H), 4.53-4.58 (m, 2H), 4.15-4.20 (m, 2H). LC-MS: m/z 230 [M+H]⁺

### Step 4: (R)-2-chloro-N-(5-(difluoromethyl)-6-(2-oxooxazolidin-3-yl)pyridin-3-yl)-8-methyl-8-(trifluoromethyl)-7,8-dihydro-6H-pyrazolo[1,5-a]pyrrolo[2,3-e]pyrimidine-6-carboxamide

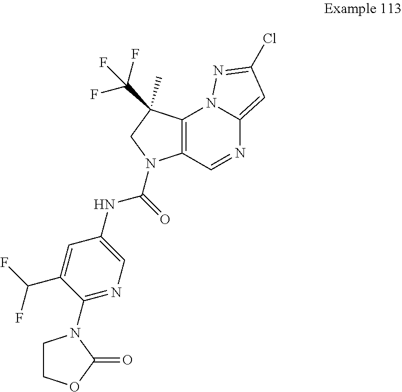 [SMILES: C[C@@]1(C(F)(F)F)CN(C(=O)Nc2cnc(N3CCOC3=O)c(C(F)F)c2)c2cnc3cc(Cl)nn3c21]

To a mixture of Method M1 isomer 2 (70 mg, 254.3 μmol) in tetrahydrofuran (2 mL) were added triphosgene (45 mg, 152.2 μmol) and TEA (38 mg, 382.4 μmol) at 25° C. The reaction mixture was stirred at 25° C. for 0.5 h and then filtered. The filtrate was added to a solution of 3-(5-amino-3-(difluoromethyl)pyridin-2-yl)oxazolidin-2-one (70 mg, 305.1 μmol) in tetrahydrofuran (2 mL). To this solution were added TEA (257 mg, 2.5 mmol) and N,N-dimethylpyridin-4-amine (62 mg, 509.6 μmol). The reaction mixture was stirred at 40° C. for 2 h. The reaction mixture was quenched with water (20 mL). The resulting solution was extracted with ethyl acetate (3×20 mL). The combined organic layers were dried over anhydrous sodium sulfate and concentrated under vacuum. The residue was submitted to Prep-HPLC purification and the collected fractions were lyophilized to afford (R)-2-chloro-N-(5-(difluoromethyl)-6-(2-oxooxazolidin-3-yl)pyridin-3-yl)-8-methyl-8-(trifluoromethyl)-7,8-dihydro-6H-pyrazolo[1,5-a]pyrrolo[2,3-e]pyrimidine-6-carboxamide (6 mg, 7% yield) as a white solid. The enantiomer of Example 113 can be prepared analogously using Method M1 isomer 1.

Example 113: ¹H NMR (300 MHz, DMSO-d₆) δ: 9.50 (br, 1H), 9.36 (s, 1H), 8.82-8.83 (m, 1H), 8.37-8.38 (m, 1H), 7.17 (t, J=54.3 Hz, 1H), 7.07 (s, 1H), 4.84 (d, J=11.4 Hz, 1H), 4.52-4.57 (m, 2H), 4.29 (d, J=11.4 Hz, 1H), 4.14-4.19 (m, 2H), 1.99 (s, 3H). LC-MS: m/z 532 [M+H]⁺.

### Method D4

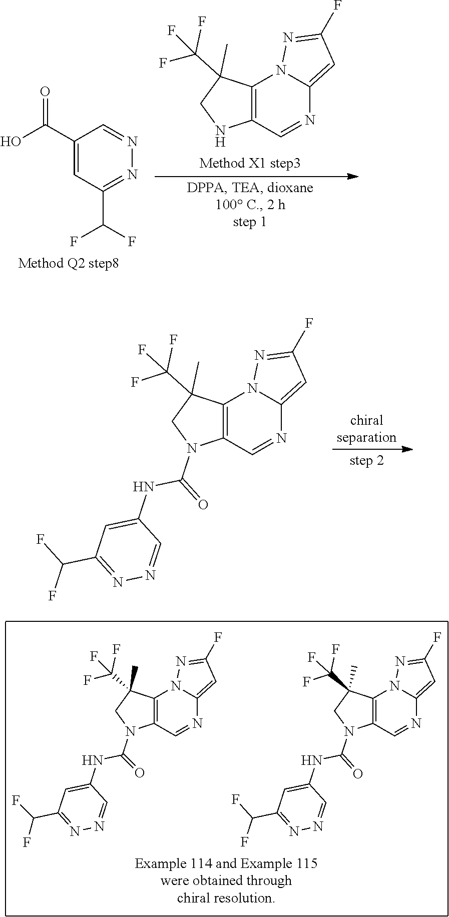 [SMILES: O=C(O)c1cnnc(C(F)F)c1 ; CC1(C(F)(F)F)CNc2cnc3cc(F)nn3c21 ; CC1(C(F)(F)F)CN(C(=O)Nc2cnnc(C(F)F)c2)c2cnc3cc(F)nn3c21 ; C[C@]1(C(F)(F)F)CN(C(=O)Nc2cnnc(C(F)F)c2)c2cnc3cc(F)nn3c21 ; C[C@@]1(C(F)(F)F)CN(C(=O)Nc2cnnc(C(F)F)c2)c2cnc3cc(F)nn3c21]

### Example 114 and 115: Single enantiomers obtained from a racemic mixture containing (S)—N-(6-(difluoromethyl)pyridazin-4-yl)-2-fluoro-8-methyl-8-(trifluoromethyl)-7,8-dihydro-6H-pyrazolo[1,5-a]pyrrolo[2,3-e]pyrimidine-6-carboxamide and (R)—N-(6-(difluoromethyl)pyridazin-4-yl)-2-fluoro-8-methyl-8-(trifluoromethyl)-7,8-dihydro-6H-pyrazolo[1,5-a]pyrrolo[2,3-e]pyrimidine-6-carboxamide

### Step 1: N-(6-(difluoromethyl)pyridazin-4-yl)-2-fluoro-8-methyl-8-(trifluoromethyl)-7,8-dihydro-6H-pyrazolo[1,5-a]pyrrolo[2,3-e]pyrimidine-6-carboxamide

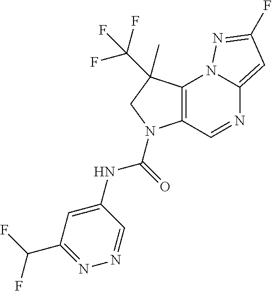 [SMILES: CC1(C(F)(F)F)CN(C(=O)Nc2cnnc(C(F)F)c2)c2cnc3cc(F)nn3c21]

To a stirred solution of 6-(difluoromethyl)pyridazine-4-carboxylic acid (Method Q2 step 8; 40 mg, 229.8 μmol) in dioxane (10 mL) were added DPPA (75.9 mg, 275.7 μmol), TEA (69.7 mg, 689.2 μmol) and 2-fluoro-8-methyl-8-(trifluoromethyl)-7,8-dihydro-6H-pyrazolo[1,5-a]pyrrolo[2,3-e]pyrimidine (Method X1 step 3; 59.8 mg, 229.8 μmol). The reaction mixture was stirred at 100° C. for 2 h. The mixture was cooled to 25° C. The mixture was concentrated under vacuum. The residue was purified by Prep-TLC using 97% dichloromethane and 3% methanol as eluents to afford 80 mg of crude product. The crude product was purified by Prep-HPLC and the collected fractions were lyophilized to afford N-(6-(difluoromethyl)pyridazin-4-yl)-2-fluoro-8-methyl-8-(trifluoromethyl)-7,8-dihydro-6H-pyrazolo[1,5-a]pyrrolo[2,3-e]pyrimidine-6-carboxamide (16 mg, 16.1% yield) as a white solid. ¹H NMR (400 MHz, DMSO-d₆) δ: 9.91 (br, 1H), 9.48 (s, 1H), 9.34 (s, 1H), 8.21 (d, J=2.8 Hz, 1H), 7.23 (t, J=54.2 Hz, 1H), 6.70 (d, J=4.8 Hz, 1H), 4.85 (d, J=11.6 Hz, 1H), 4.30 (d, J=11.6 Hz, 1H), 1.96 (s, 3H). LC-MS: m/z 432 [M+H]⁺.

### Step 2: Separation of enantiomers to obtain (S)—N-(6-(difluoromethyl)pyridazin-4-yl)-2-fluoro-8-methyl-8-(trifluoromethyl)-7,8-dihydro-6H-pyrazolo[1,5-a]pyrrolo[2,3-e]pyrimidine-6-carboxamide and (R)—N-(6-(difluoromethyl)pyridazin-4-yl)-2-fluoro-8-methyl-8-(trifluoromethyl)-7,8-dihydro-6H-pyrazolo[1,5-a]pyrrolo[2,3-e]pyrimidine-6-carboxamide

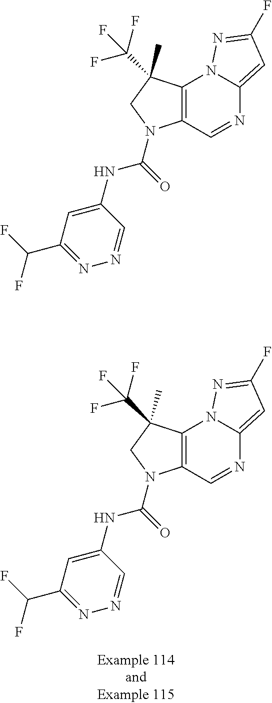 [SMILES: C[C@]1(C(F)(F)F)CN(C(=O)Nc2cnnc(C(F)F)c2)c2cnc3cc(F)nn3c21 ; C[C@@]1(C(F)(F)F)CN(C(=O)Nc2cnnc(C(F)F)c2)c2cnc3cc(F)nn3c21]

N-(6-(difluoromethyl)pyridazin-4-yl)-2-fluoro-8-methyl-8-(trifluoromethyl)-7,8-dihydro-6H-pyrazolo[1,5-a]pyrrolo[2,3-e]pyrimidine-6-carboxamide (16 mg, 37.1 μmol) was submitted to chiral-HPLC (Column: CHIRAL ART Cellulose-SB, 2×25 cm, Sum; Mobile Phase A: Hex (0.5% 2M NH₃-MeOH)—HPLC, Mobile Phase B: EtOH—HPLC; Flow rate: 20 mL/min; Gradient: 15 B to 15 B in 19 min; 220/254 nm; RT1:13.572; RT2:16.226; Injection Volume: 0.8 ml; Number of Runs: 6). The first eluting isomer was concentrated and lyophilized to afford Example 114 (5.9 mg, 36% yield) as a white solid. The second eluting isomer was concentrated and lyophilized to afford Example 115 (5.1 mg, 32% yield) as a white solid. Examples 114 and 115 are enantiomers, but their absolute stereochemistry is not yet known.

Example 114: ¹H NMR (400 MHz, DMSO-d₆) δ: 9.91 (br, 1H), 9.50 (d, J=2.4 Hz, 1H), 9.33 (s, 1H), 8.21 (d, J=2.8 Hz, 1H), 7.24 (t, J=56.0 Hz, 1H), 6.71 (d, J=5.2 Hz, 1H), 4.86 (d, J=11.2 Hz, 1H), 4.30 (d, J=11.2 Hz, 1H), 1.96 (s, 3H). LC-MS: m/z 432 [M+H]⁺.

Example 115: ¹H NMR (400 MHz, DMSO-d₆) δ: 9.91 (br, 1H), 9.50 (d, J=2.4 Hz, 1H), 9.33 (s, 1H), 8.21 (d, J=2.8 Hz, 1H), 7.24 (t, J=54.4 Hz, 1H), 6.71 (d, J=4.8 Hz, 1H), 4.86 (d, J=11.2 Hz, 1H), 4.30 (d, J=11.2 Hz, 1H), 1.96 (s, 3H). LC-MS: m/z 432 [M+H]⁺.

### Method E4

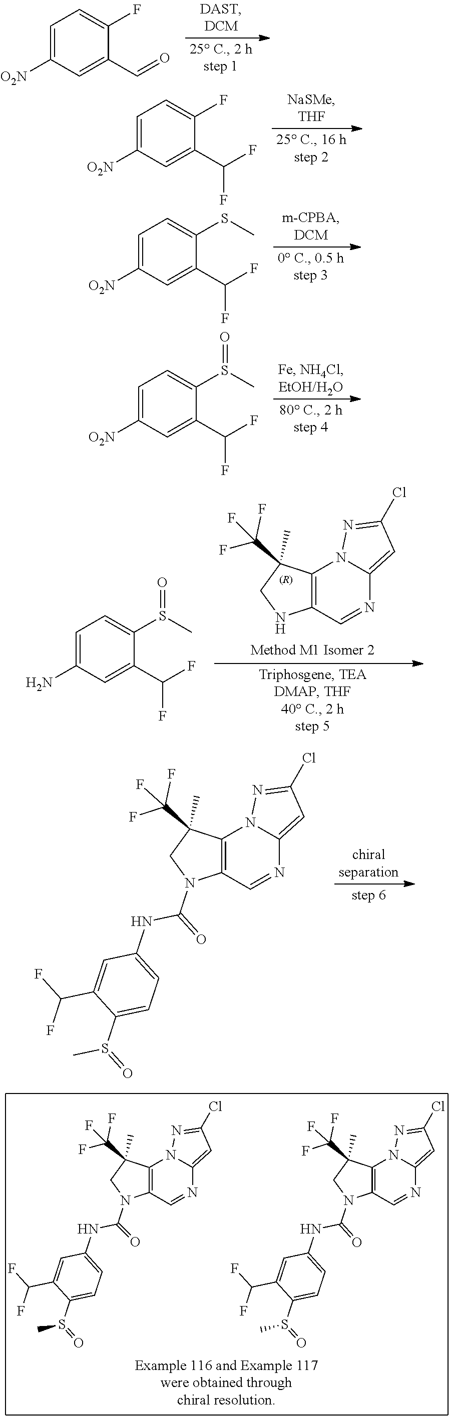

### Examples 116 and 117: (R)-2-chloro-N-(3-(difluoromethyl)-4-((S)-methylsulfanyl)phenyl)-8-methyl-8-(trifluoromethyl)-7,8-dihydro-6H-pyrazolo[1,5-a]pyrrolo[2,3-e]pyrimidine-6-carboxamide and (R)-2-chloro-N-(3-(difluoromethyl)-4-((R)-methylsulfinyl)phenyl)-8-methyl-8-(trifluoromethyl)-7,8-dihydro-6H-pyrazolo[1,5-a]pyrrolo[2,3-e]pyrimidine-6-carboxamide

### Step 1: 2-(difluoromethyl)-1-fluoro-4-nitrobenzene

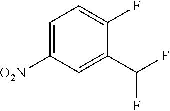 [SMILES: O=[N+]([O-])c1ccc(F)c(C(F)F)c1]

To a stirred solution of 2-fluoro-5-nitrobenzaldehyde (10.0 g, 59.1 mmol) in dichloromethane (100 mL) was added DAST (19.0 g, 118.2 mmol) dropwise at 0° C. The mixture was stirred at 25° C. for 2 h. The pH was adjusted to 7-8 with saturated aqueous NaHCO₃ solution. The resulted mixture was extracted with dichloromethane (3×200 mL). The combined organic layers were dried over anhydrous sodium sulfate and concentrated under vacuum. The residue was purified by column chromatography on silica gel using 70% petroleum ether and 30% ethyl acetate as eluents to afford 2-(difluoromethyl)-1-fluoro-4-nitrobenzene (10.0 g, 88% yield) as a yellow oil. ¹H NMR (400 MHz, Chloroform-d) δ: 8.51-8.56 (m, 1H), 8.40-8.44 (m, 1H), 7.36 (t, J=9.2 Hz, 1H), 6.93 (t, J=54.4 Hz, 1H).

### Step 2: (2-(difluoromethyl)-4-nitrophenyl)(methyl)sulfane

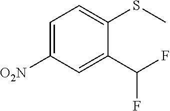

To a stirred solution of 2-(difluoromethyl)-1-fluoro-4-nitrobenzene (4.0 g, 20.9 mmol) in tetrahydrofuran (100 mL) was added sodium thiomethoxide (1.4 g, 20.9 mmol) at 0° C. under nitrogen atmosphere. The mixture was stirred at 25° C. for 16 h. The solid was collected by filtration. The filter cake was washed with ethyl acetate (2×100 mL). The crude product was purified by column chromatography on silica gel using 75% petroleum ether and 25% ethyl acetate as eluents to afford (2-(difluoromethyl)-4-nitrophenyl)(methyl)sulfane (3.2 g, 69% yield) as a yellow solid. H NMR (400 MHz, Chloroform-d) δ: 8.44 (d, J=2.8 Hz, 1H), 8.27 (dd, J=2.8, 8.8 Hz, 1H), 7.41 (d, J=8.8 Hz, 1H), 6.89 (t, J=54.4 Hz, 1H), 2.62 (s, 3H).

### Step 3: 2-(difluoromethyl)-1-(methylsulfinyl)-4-nitrobenzene

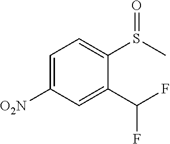

To a stirred solution of (2-(difluoromethyl)-4-nitrophenyl)(methyl)sulfane (1.0 g, 4.5 mmol) in dichloromethane (100 mL) was added 3-chlorobenzoperoxoic acid (787 mg, 4.5 mmol) at 0° C. The mixture was stirred at 0° C. for 0.5 h. The mixture was concentrated under vacuum. The residue was purified by column chromatography on silica gel using 50% petroleum ether and 50% ethyl acetate as eluents to afford 2-(difluoromethyl)-1-(methylsulfinyl)-4-nitrobenzene (850 mg, 79% yield) as a white solid. ¹H NMR (400 MHz, Chloroform-d) δ: 8.55-8.59 (m, 1H), 8.51-8.52 (m, 1H), 8.42 (d, J=8.4 Hz, 1H), 6.99 (t, J=54.8 Hz, 1H), 2.83 (s, 3H). LC-MS: m/z 236 [M+H]⁺.

### Step 4: 3-(difluoromethyl)-4-(methylsulfinyl)aniline

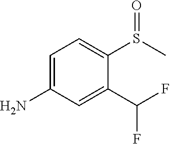

To a stirred solution of 2-(difluoromethyl)-1-(methylsulfinyl)-4-nitrobenzene (850 mg, 3.6 mmol) in ethanol (15 mL) and water (5 mL) were added Fe (605 mg, 10.8 mmol) and NH₄Cl (966 mg, 18.0 mmol). The mixture was stirred at 80° C. for 2 h. After cooled to 25° C., the solid was filtered out. The filtrate was quenched with water (50 mL). The resulting mixture was extracted with ethyl acetate (3×50 mL). The combined organic layers were dried over anhydrous sodium sulfate and concentrated under vacuum. The residue was purified by column chromatography on silica gel using 95% dichloromethane and 5% methanol as eluents to afford 3-(difluoromethyl)-4-(methylsulfinyl)aniline (540 mg, 72% yield) as a yellow solid. ¹H NMR (400 MHz, Chloroform-d) δ: 7.83-7.90 (m, 1H), 6.74-7.09 (m, 3H), 4.16 (br, 2H), 2.71 (s, 3H). LC-MS: m/z 206 [M+H]⁺.

### Step 5: (8R)-2-chloro-N-(3-(difluoromethyl)-4-(methylsulfinyl)phenyl)-8-methyl-8-(trifluoromethyl)-7,8-dihydro-6H-pyrazolo[1,5-a]pyrrolo[2,3-e]pyrimidine-6-carboxamide

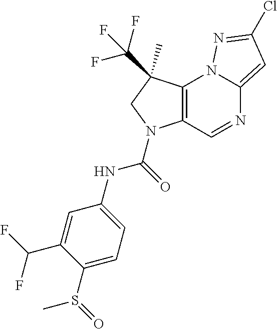 [SMILES: CS(=O)c1ccc(NC(=O)N2C[C@@](C)(C(F)(F)F)c3c2cnc2cc(Cl)nn32)cc1C(F)F]

To a stirred solution of 3-(difluoromethyl)-4-(methylsulfinyl)aniline (100 mg, 487.8 μmol) in tetrahydrofuran (40 mL) were added triphosgene (86 mg, 292.7 μmol) and TEA (73 mg, 722.7 μmol) at 0° C. The resulting mixture was stirred at 25° C. for 0.5 h and then filtered. The filtrate was added to a solution of Method M1 isomer 2 (134 mg, 487.8 μmol) in tetrahydrofuran (2 mL). To this solution were added N,N-dimethylpyridin-4-amine (119 mg, 975.6 μmol) and TEA (493 mg, 4.8 mmol). The mixture was stirred at 40° C. for 2 h. The solvent was concentrated under vacuum. The residue was submitted to Prep-HPLC purification and the collected fractions were lyophilized to afford (8R)-2-chloro-N-(3-(difluoromethyl)-4-(methylsulfinyl)phenyl)-8-methyl-8-(trifluoromethyl)-7,8-dihydro-6H-pyrazolo[1,5-a]pyrrolo[2,3-e]pyrimidine-6-carboxamide (140 mg, 56% yield) as a white solid.

### Step 6: Separation of enantiomers to obtain (R)-2-chloro-N-(3-(difluoromethyl)-4-((S)-methylsulfinyl)phenyl)-8-methyl-8-(trifluoromethyl)-7,8-dihydro-6H-pyrazolo[1,5-a]pyrrolo[2,3-e]pyrimidine-6-carboxamide and (R)-2-chloro-N-(3-(difluoromethyl)-4-((R)-methylsulfinyl)phenyl)-8-methyl-8-(trifluoromethyl)-7,8-dihydro-6H-pyrazolo[1,5-a]pyrrolo[2,3-e]pyrimidine-6-carboxamide

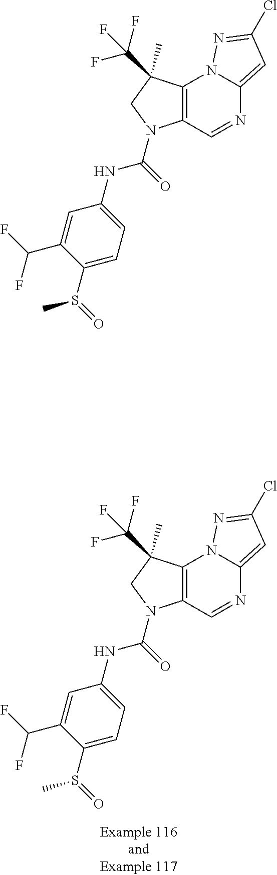 [SMILES: C[S@](=O)c1ccc(NC(=O)N2C[C@@](C)(C(F)(F)F)c3c2cnc2cc(Cl)nn32)cc1C(F)F ; C[S@@](=O)c1ccc(NC(=O)N2C[C@@](C)(C(F)(F)F)c3c2cnc2cc(Cl)nn32)cc1C(F)F]

(8R)-2-chloro-N-(3-(difluoromethyl)-4-(methylsulfinyl)phenyl)-8-methyl-8-(trifluoromethyl)-7,8-dihydro-6H-pyrazolo[1,5-a]pyrrolo[2,3-e]pyrimidine-6-carboxamide (140 mg, 275.5 μmol) was submitted to chiral-HPLC: Column: CHIRAL ART Cellulose-SB, 2×25 cm, 5 um; Mobile Phase A: MTBE (0.5% 2M NH₃-methanol)-HPLC, Mobile Phase B: IPA-HPLC; Flow rate: 20 mL/min; Gradient: 10 B to 10 B in 35 min; 220/254 nm; RT1: 25.605; RT2: 28.879; Injection Volume: 0.5 ml; Number Of Runs: 5. The first eluting isomer was concentrated and lyophilized to afford Example 116 (45.2 mg, 32% yield) as a white solid. The second eluting isomer was concentrated and lyophilized to afford Example 117 (42.5 mg, 30% yield) as a white solid. The corresponding stereoisomers of Example 116 and Example 117 can be prepared analogously using Method M1 isomer 1 in step 5. Examples 116 and 117 are diastereomers, wherein the stereocenter attached to the trifluoromethyl is absolute and the sulfoxide stereocenter is relative (i.e., the sulfoxide stereocenter in one of Example 116 and 117 is (S), and the sulfoxide stereocenter in the other of Example 116 and 117 is (R)).

Example 116: ¹H NMR (400 MHz, DMSO-d₆) δ: 9.47 (s, 1H), 9.34 (s, 1H), 7.99-8.06 (m, 3H), 7.37 (t, J=54.8 Hz, 1H), 7.06 (s, 1H), 4.86 (d, J=11.6 Hz, 1H), 4.27 (d, J=11.6 Hz, 1H), 2.73 (s, 3H), 1.97 (s, 3H). LC-MS: m/z 508 [M+H]⁺.

Example 117: ¹H NMR (400 MHz, DMSO-d₆) δ: 9.47 (s, 1H), 9.34 (s, 1H), 7.98-8.10 (m, 3H), 7.37 (t, J=54.8 Hz, 1H), 7.06 (s, 1H), 4.87 (d, J=11.6 Hz, 1H), 4.27 (d, J=11.6 Hz, 1H), 2.73 (s, 3H), 1.97 (s, 3H). LC-MS: m/z 508 [M+H]⁺.

### Method F4

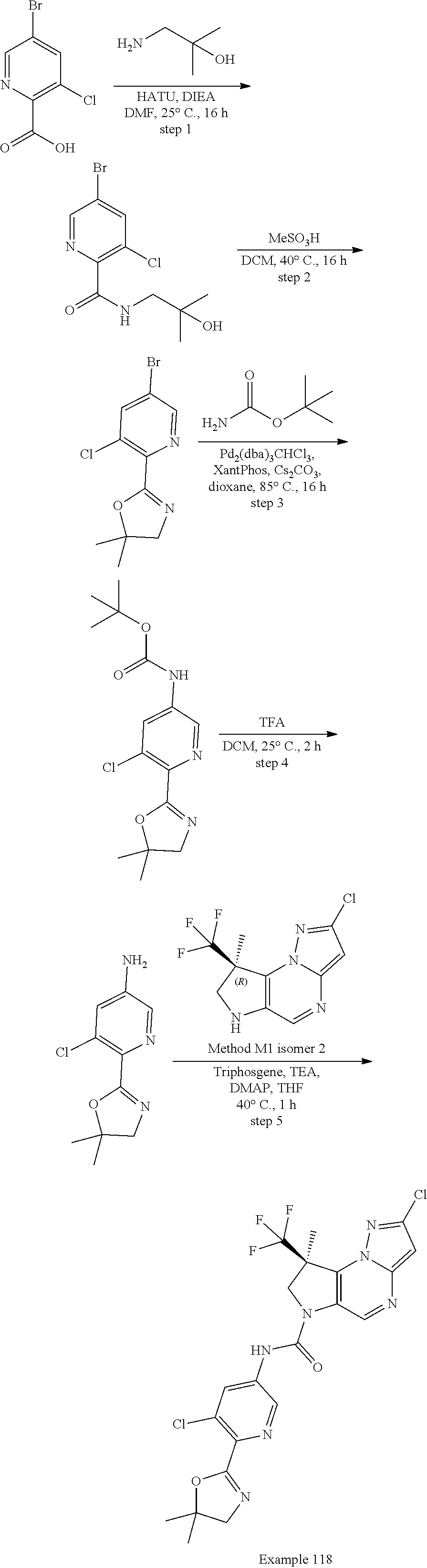

Examples 118: (R)-2-chloro-N-(5-chloro-6-(5,5-dimethyl-4,5-dihydrooxazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide Step 1: 5-bromo-3-chloro-N-(2-hydroxy-2-methylpropyl)picolinamide

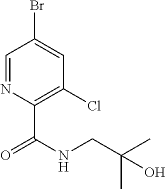 [SMILES: CC(C)(O)CNC(=O)c1ncc(Br)cc1Cl]

To a stirred solution of 5-bromo-3-chloropicolinic acid (10.0 g, 42.6 mmol) in N,N-Dimethylformamide (200 mL) were added 1-amino-2-methylpropan-2-ol (3.8 g, 42.6 mmol), HATU (26.2 g, 63.8 mmol) and DIEA (16.5 g, 127.7 mmol). The reaction mixture was stirred at 25° C. for 16 h. The reaction mixture was concentrated under vacuum. The residue was purified by column chromatography on silica gel using 10% methanol and 90% dichloromethane as eluents to afford 5-bromo-3-chloro-N-(2-hydroxy-2-methylpropyl)picolinamide (1.0 g, 72% yield) as a yellow oil. ¹H NMR (400 MHz, Chloroform-d) δ: 8.52 (d, J=4 Hz, 1H), 8.00 (d, J=4 Hz, 1H), 3.46 (d, J=4 Hz, 2H), 1.29 (s, 6H). LC-MS: m/z 307 [M+H]⁺.

### Step 2: 2-(5-bromo-3-chloropyridin-2-yl)-5,5-dimethyl-4,5-dihydrooxazole

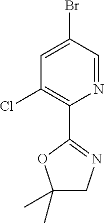

To a stirred solution of 5-bromo-3-chloro-N-(2-hydroxy-2-methylpropyl)picolinamide (5.0 g, 16.3 mmol) in dichloromethane (80 mL) was added methanesulfonic acid (7.8 g, 81.5 mmol). The reaction mixture was stirred at 40° C. for 16 h. The mixture was allowed to cool down to 25° C. The reaction mixture was quenched with saturated NaHCO₃ solution (200 mL). The resulting solution was extracted with ethyl acetate (3×200 mL). The combined organic layers were dried over anhydrous sodium sulfate and concentrated under vacuum. The residue was purified by column chromatography on silica gel using 50% petroleum ether and 50% ethyl acetate as eluent to afford 2-(5-bromo-3-chloropyridin-2-yl)-5,5-dimethyl-4,5-dihydrooxazole (2.0 g, 42% yield) as a yellow oil. ¹H NMR (400 MHz, Chloroform-d) δ: 8.68 (d, J=2 Hz, 1H), 8.01 (d, J=2 Hz, 1H), 3.91 (s, 2H), 1.57 (s, 6H). LC-MS: m/z 289 [M+H]⁺.

### Step 3: tert-butyl (5-chloro-6-(5,5-dimethyl-4,5-dihydrooxazol-2-yl)pyridin-3-yl)carbamate

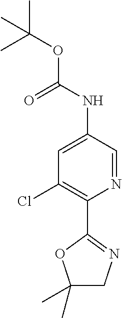 [SMILES: CC(C)(C)OC(=O)Nc1cnc(C2=NCC(C)(C)O2)c(Cl)c1]

To a stirred solution of 2-(5-bromo-3-chloropyridin-2-yl)-5,5-dimethyl-4,5-dihydrooxazole (1 g, 3.5 mmol) in dioxane (30 mL) were added tert-butyl carbamate (1.6 g, 14 mmol), Pd₂(dba)₃CHCl₃ (0.4 g, 0.3 mmol), XantPhos (0.4 g, 0.6 mmol) and Cs₂CO₃ (2.3 g, 7 mmol) under nitrogen atmosphere. The resulting mixture was stirred for 16 h at 85° C. The mixture was allowed to cool down to 25° C. The resulting solution was concentrated under vacuum. The residue was purified by column chromatography on silica gel using 10% methanol and 90% dichloromethane as eluent to afford tert-butyl (5-chloro-6-(5,5-dimethyl-4,5-dihydrooxazol-2-yl)pyridin-3-yl)carbamate (1 g, 72% yield) as a yellow oil. ¹H NMR (400 MHz, Chloroform-d) δ: 8.32-8.33 (m, 2H), 3.87 (s, 2H), 1.53 (s, 6H), 1.52 (s, 9H). LC-MS: m/z 326 [M+H]⁺.

### Step 4: 5-chloro-6-(5,5-dimethyl-4,5-dihydrooxazol-2-yl)pyridin-3-amine

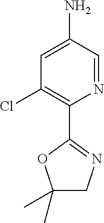

To a solution of 5-chloro-6-(5,5-dimethyl-4,5-dihydrooxazol-2-yl)pyridin-3-amine (500 mg, 1.5 mmol) in dichloromethane (20 mL) was added TFA (4 mL). The resulting mixture was stirred at 25° C. for 2 h. The mixture was concentrated under vacuum. The pH was adjusted to 8 with saturated aqueous NaHCO₃ solution. The resulting mixture was extracted with ethyl acetate (3×50 mL). The combined organic layers were dried over anhydrous sodium sulfate and concentrated under vacuum. The residue was purified by column chromatography on silica gel using 20% methanol and 80% dichloromethane as eluents to afford 5-chloro-6-(5,5-dimethyl-4,5-dihydrooxazol-2-yl)pyridin-3-amine (300 mg, 86% yield) as a yellow solid. ¹H NMR (300 MHz, DMSO-d₆) δ: 7.88 (s, 1H), 6.99 (s, 1H), 6.09 (br, 2H), 3.66 (s, 2H), 1.39 (s, 6H); LC-MS: m/z 226 [M+H]⁺.

### Step 5: (R)-2-chloro-N-(5-chloro-6-(5,5-dimethyl-4,5-dihydrooxazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

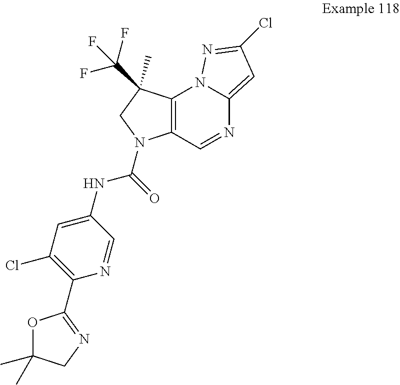 [SMILES: CC1(C)CN=C(c2ncc(NC(=O)N3C[C@@](C)(C(F)(F)F)c4c3cnc3cc(Cl)nn43)cc2Cl)O1]

To a stirred solution of Method M1 isomer 2 (30 mg, 108.6 μmol) in tetrahydrofuran (6 mL) were added triphosgene (20 mg, 65.2 μmol) and TEA (17 mg, 163 μmol) at 0° C. The resulting mixture was stirred at 28° C. for 0.5 h and then filtered. The filtrate was added to a solution of 5-chloro-6-(5,5-dimethyl-4,5-dihydrooxazol-2-yl)pyridin-3-amine (39 mg, 173 μmol) in tetrahydrofuran (1 mL). To this solution was then added TEA (110 mg, 1.1 mmol) and N,N-dimethylpyridin-4-amine (27 mg, 217.4 μmol). The mixture was stirred at 40° C. for 1 h. The reaction mixture was quenched with water (50 mL). The resulting solution was extracted with ethyl acetate (3×50 mL). The combined organic layers were dried over anhydrous sodium sulfate and concentrated under vacuum. The residue was purified by column chromatography on silica gel using 10% methanol and 90% dichloromethane as eluents to afford crude product. The crude product was purified by Prep-HPLC and the collected fractions were lyophilized to give (R)-2-chloro-N-(5-chloro-6-(5,5-dimethyl-4,5-dihydrooxazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide (4 mg, 6% yield) as a white solid. The enantiomer of Example 118 can be prepared analogously using Method M1 isomer 1.

Example 118: ¹H NMR (400 MHz, DMSO-d₆) δ: 9.58 (s, 1H), 9.35 (s, 1H), 8.73 (d, J=8 Hz, 1H), 8.28 (d, J=32 Hz, 1H), 7.07 (s, 1H), 4.84 (d, J=12 Hz, 1H), 4.28 (d, J=12 Hz, 1H), 3.72 (s, 2H), 1.97 (s, 3H), 1.60 (s, 6H). LC-MS: m/z 528 [M+H]⁺.

### Method G4

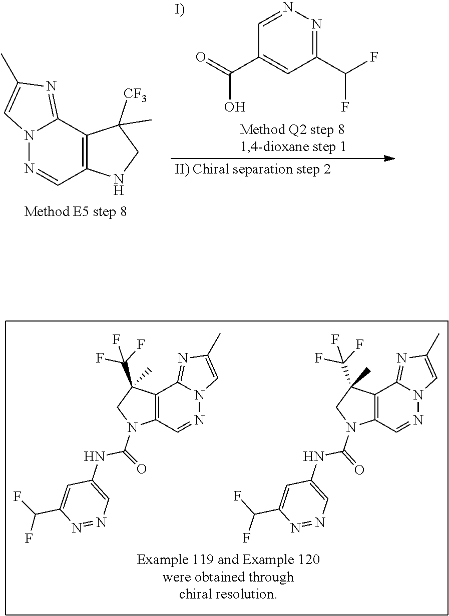

### Example 119 and 120: Single enantiomers obtained from a racemic mixture containing (S)—N-(6-(difluoromethyl)pyridazin-4-yl)-2,9-dimethyl-9-(trifluoromethyl)-8,9-dihydro-7H-imidazo[1,2-b]pyrrolo[3,2-d]pyridazine-7-carboxamide and (R)—N-(6-(difluoromethyl)pyridazin-4-yl)-2,9-dimethyl-9-(trifluoromethyl)-8,9-dihydro-7H-imidazo[1,2-b]pyrrolo[3,2-d]pyridazine-7-carboxamide

### Step 1: N-(6-(difluoromethyl)pyridazin-4-yl)-2,9-dimethyl-9-(trifluoromethyl)-8,9-dihydro-7H-imidazo[1,2-b]pyrrolo[3,2-d]pyridazine-7-carboxamide

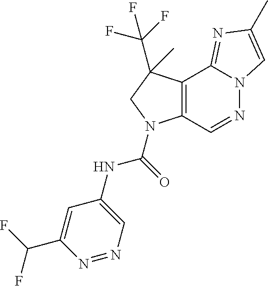 [SMILES: Cc1cn2ncc3c(c2n1)C(C)(C(F)(F)F)CN3C(=O)Nc1cnnc(C(F)F)c1]

To 2,9-dimethyl-9-(trifluoromethyl)-8,9-dihydro-7H-imidazo[1,2-b]pyrrolo[3,2-d]pyridazine (Method E5 step 8; 35 mg, 0.137 mmol) were added 6-(difluoromethyl)pyridazine-4-carboxylic acid (Method Q2 step 8; 23.78 mg, 0.137 mmol) and 1,4-dioxane (extra dry) (3 mL). To the resulting solution were added triethylamine (0.094 mL, 0.673 mmol) and diphenylphosphoryl azide (0.036 mL, 0.164 mmol). The mixture was heated to 100° C. and stirred for 2 hours. The reaction mixture was concentrated under reduced pressure. The residue (140 mg) was dissolved in DMSO and purified by chromatography to obtain N-(6-(difluoromethyl)pyridazin-4-yl)-2,9-dimethyl-9-(trifluoromethyl)-8,9-dihydro-7H-imidazo[1,2-b]pyrrolo[3,2-d]pyridazine-7-carboxamide (41 mg). LC-MS: m/z 428 [M+H]⁺.

### Step 10: Separation of enantiomers to obtain (S)—N-(6-(difluoromethyl)pyridazin-4-yl)-2,9-dimethyl-9-(trifluoromethyl)-8,9-dihydro-7H-imidazo[1,2-b]pyrrolo[3,2-d]pyridazine-7-carboxamide and (R)—N-(6-(difluoromethyl)pyridazin-4-yl)-2,9-dimethyl-9-(trifluoromethyl)-8,9-dihydro-7H-imidazo[1,2-b]pyrrolo[3,2-d]pyridazine-7-carboxamide

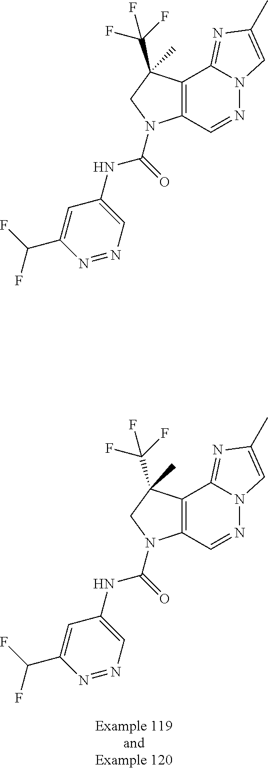 [SMILES: Cc1cn2ncc3c(c2n1)[C@](C)(C(F)(F)F)CN3C(=O)Nc1cnnc(C(F)F)c1 ; Cc1cn2ncc3c(c2n1)[C@@](C)(C(F)(F)F)CN3C(=O)Nc1cnnc(C(F)F)c1]

N-(6-(difluoromethyl)pyridazin-4-yl)-2,9-dimethyl-9-(trifluoromethyl)-8,9-dihydro-7H-imidazo[1,2-b]pyrrolo[3,2-d]pyridazine-7-carboxamide (41 mg) was submitted to chiral-SFC (Column: Phenomenex Cellulose-2, 4.6×100 mm, 5 μm; Mobile Phase A: C02, Mobile Phase B:iPrOH 20 mM Ammonia-HPLC; Flow rate: 2.5 mL/min; Gradient: 5 B to 50% B in 5 min; 210-320 nm; RT1:3.434; RT2:3.822). The first eluting isomer was concentrated and lyophilized to afford Example 119 (15.4 mg, 26% yield) and the second eluting isomer was concentrated and lyophilized to afford Example 120 (15.3 mg, 26.2% yield). Examples 119 and 120 are enantiomers, but their absolute stereochemistry is not yet known.

Example 119: ¹H NMR (400 MHz, CDCl₃) δ: 9.31 (d, J=2.6 Hz, 1H), 9.29 (s, 1H), 8.39 (d, J=2.5 Hz, 1H), 7.79 (s, 2H), 6.88 (t, J=54.6 Hz, 1H), 4.66 (d, J=10.5 Hz, 1H), 4.08 (d, J=10.5 Hz, 1H), 2.51 (s, 3H), 1.98 (s, 3H). LC-MS: m/z 428 [M+H]⁺.

Example 120: ¹H NMR (400 MHz, CDCl₃) δ: 9.32 (d, J=2.6 Hz, 1H), 9.29 (s, 1H), 8.36 (d, J=2.5 Hz, 1H), 7.79 (s, 1H), 7.41 (s, 1H), 6.89 (t, J=54.5 Hz, 1H), 4.62 (d, J=10.5 Hz, 1H), 4.06 (d, J=10.5 Hz, 1H), 2.52 (s, 3H), 2.00 (s, 3H). LC-MS: m/z 428 [M+H]⁺.

### Method H4

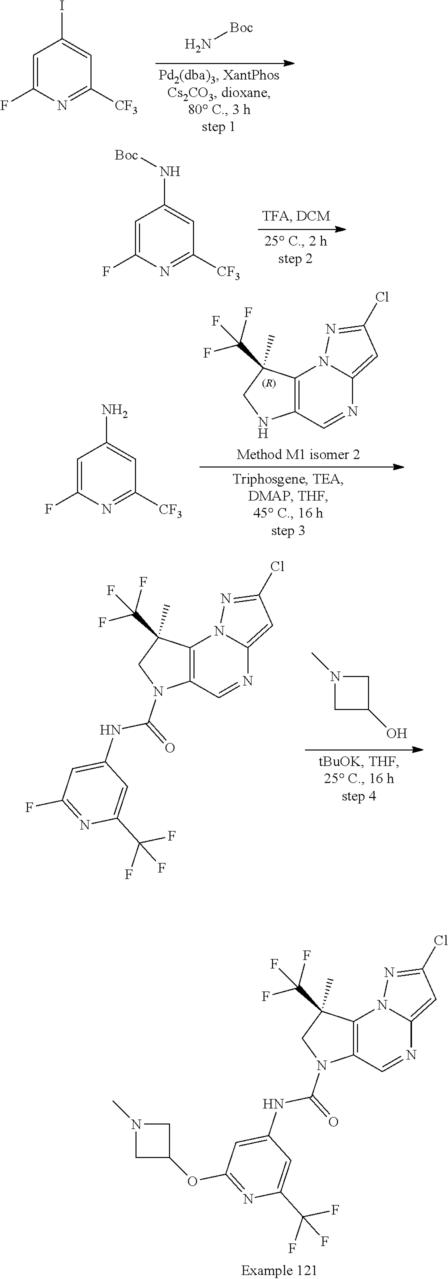

### Example 121: (R)-2-chloro-8-methyl-N-(2-((1-methylazetidin-3-yl)oxy)-6-(trifluoromethyl)pyridin-4-yl)-8-(trifluoromethyl)-7,8-dihydro-6H-pyrazolo[1,5-a]pyrrolo[2,3-e]pyrimidine-6-carboxamide

### Step 1: tert-butyl (2-fluoro-6-(trifluoromethyl)pyridin-4-yl)carbamate

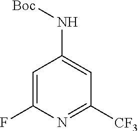 [SMILES: CC(C)(C)OC(=O)Nc1cc(F)nc(C(F)(F)F)c1]

To a stirred solution of 2-fluoro-4-iodo-6-(trifluoromethyl)pyridine (500 mg, 1.7 mmol) and tert-butyl carbamate (302 mg, 2.6 mmol) in dioxane (10 mL) were added Xantphos (198 mg, 342 μmol), Pd₂(dba)₃ (117 mg, 171 μmol) and Cs₂CO₃ (1.1 g, 3.4 mmol) under nitrogen atmosphere. The resulting mixture was stirred for 3 h at 80° C. The reaction was cooled to 25° C. The solid was filtered out. The filtrate was concentrated under vacuum. The residue was purified by column chromatography on silica gel using 80% petroleum ether and 20% ethyl acetate as eluents to afford tert-butyl (2-fluoro-6-(trifluoromethyl)pyridin-4-yl)carbamate (400 mg, 70% yield) as a white solid. ¹H NMR (300 MHz, Chloroform-d) δ: 7.55-7.59 (m, 1H), 6.90 (s, 1H), 1.56 (s, 9H). LC-MS: m/z 281 [M+H]⁺.

### Step 2: 2-fluoro-6-(trifluoromethyl)pyridin-4-amine

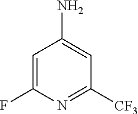 [SMILES: Nc1cc(F)nc(C(F)(F)F)c1]

To a stirred solution of tert-butyl (2-fluoro-6-(trifluoromethyl)pyridin-4-yl)carbamate (400 mg, 1.4 mmol) in dichloromethane (12 mL) was added TFA (3 mL). The mixture was stirred at 25° C. for 2 h. The resulting mixture was concentrated under vacuum. The residue was added saturated aqueous NaHCO₃ solution (40 mL). The resulting solution was extracted with dichloromethane (3×40 mL). The combined organic layers were washed with brine (50 mL) and dried over anhydrous sodium sulfate and concentrated under vacuum. The residue was purified by column chromatography on silica gel using 40% petroleum ether and 60% ethyl acetate as eluents to afford 2-fluoro-6-(trifluoromethyl)pyridin-4-amine (200 mg, 70% yield) as a white solid. ¹H NMR (400 MHz, Chloroform-d) δ: 6.79 (s, 1H), 6.22 (d, J=1.8 Hz, 1H), 4.64 (br, 2H). LC-MS: m/z 181 [M+H]⁺.

### Step 3: (R)-2-chloro-N-(2-fluoro-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide

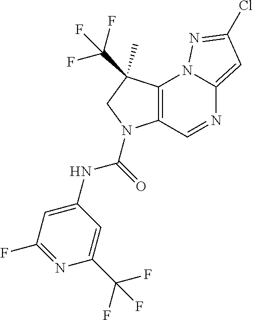 [SMILES: C[C@@]1(C(F)(F)F)CN(C(=O)Nc2cc(F)nc(C(F)(F)F)c2)c2cnc3cc(Cl)nn3c21]

To a stirred solution of Method M1 isomer 2 (50 mg, 181.2 μmol) in tetrahydrofuran (4 mL) were added triphosgene (32 mg, 108.6 μmol) and TEA (27 mg, 271.8 μmol) at 0° C. The resulting mixture was stirred at 25° C. for 0.5 h and then filtered. The filtrate was added to a solution of 2-fluoro-6-(trifluoromethyl)pyridin-4-amine (32 mg, 181.2 μmol) in tetrahydrofuran (2 mL). To this solution was then added N, N-dimethylpyridin-4-amine (20 mg, 181.2 μmol) and TEA (182 mg, 1.8 mmol). The mixture was stirred at 45° C. for 16 h. The reaction was cooled to 25° C. The mixture was poured into water (20 mL) and extracted with ethyl acetate (3×20 mL). The combined organic layers were washed with brine (50 mL), dried over anhydrous sodium sulfate and concentrated under vacuum. The residue was purified by Prep-TLC using 95% dichloromethane and 5% methanol as eluents to afford (R)-2-chloro-N-(2-fluoro-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide (30 mg, 34% yield) as a white solid. ¹H NMR (300 MHz, Methanol-d₄) δ: 7.61 (s, 1H), 7.53 (s, 1H), 6.91 (d, J=1.8 Hz, 1H), 6.79 (s, 1H), 4.61 (d, J=12 Hz, 1H), 4.21 (d, J=12 Hz, 1H), 2.07 (s, 3H). LC-MS: m/z 483 [M+H]⁺.

### Step 4: (R)-2-chloro-8-methyl-N-(2-((1-methylazetidin-3-yl)oxy)-6-(trifluoromethyl)pyridin-4-yl)-8-(trifluoromethyl)-7,8-dihydro-6H-pyrazolo[1,5-a]pyrrolo[2,3-e]pyrimidine-6-carboxamide

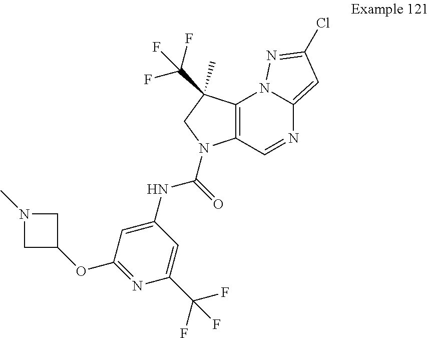 [SMILES: CN1CC(Oc2cc(NC(=O)N3C[C@@](C)(C(F)(F)F)c4c3cnc3cc(Cl)nn43)cc(C(F)(F)F)n2)C1]

To a stirred solution of (R)-2-chloro-N-(2-fluoro-6-(trifluoromethyl)pyridin-4-yl)-8-methyl-8-(trifluoromethyl)-7,8-dihydro-6H-pyrazolo[1,5-a]pyrrolo[2,3-e]pyrimidine-6-carboxamide (30 mg, 62.3 μmol) in tetrahydrofuran (5 mL) was added 1-methylazetidin-3-ol (12 mg, 133.3 μmol) and potassium tert-butoxide (15 mg, 133.3 μmol). The mixture was stirred for 16 h at 25° C. The resulting mixture was concentrated under vacuum. The residue was purified by Prep-HPLC purification and the collected fractions were lyophilized to give (R)-2-chloro-8-methyl-N-(2-((1-methylazetidin-3-yl)oxy)-6-(trifluoromethyl)pyridin-4-yl)-8-(trifluoromethyl)-7,8-dihydro-6H-pyrazolo[1,5-a]pyrrolo[2,3-e]pyrimidine-6-carboxamide (3 mg, 9% yield) as a white solid. The enantiomer of Example 121 can be prepared analogously using Method M1 isomer 1.

Example 121: ¹H NMR (300 MHz, Methanol-d₄) δ 9.35 (s, 1H), 7.66 (s, 1H), 7.35 (s, 1H), 6.79 (s, 1H), 5.21-5.23 (m, 1H), 4.81 (d, J=12 Hz, 1H), 4.20 (d, J=12 Hz, 1H), 3.83-3.85 (m, 2H), 3.13-3.24 (m, 2H), 2.42 (s, 3H), 2.02 (s, 3H). LC-MS: m/z 550 [M+H]⁺.

### Method I4

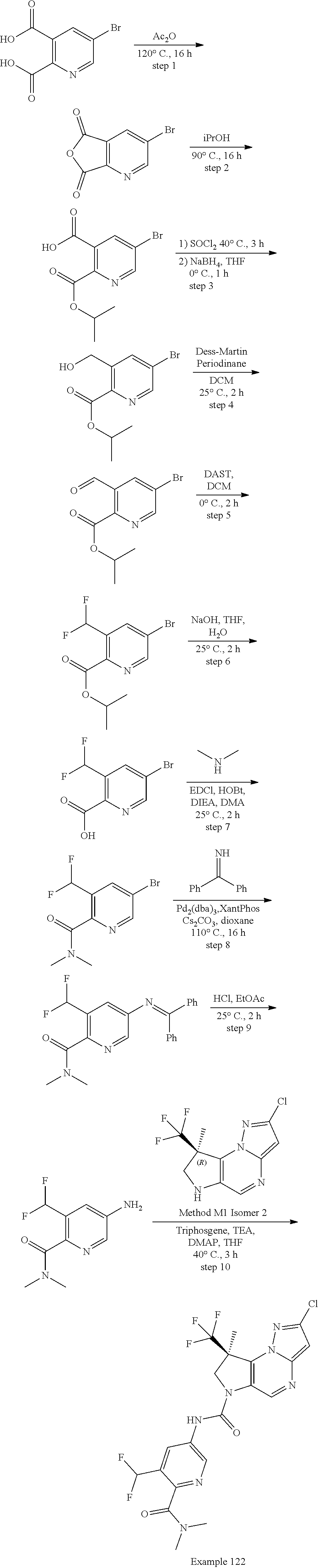

### Example 122: (R)-2-chloro-N-(5-(difluoromethyl)-6-(dimethylcarbamoyl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 3-bromofuro[3,4-b]pyridine-5,7-dione

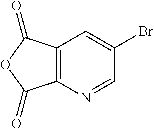 [SMILES: O=C1OC(=O)c2ncc(Br)cc21]

A solution of 5-bromopyridine-2,3-dicarboxylic acid (5.0 g, 20.3 mmol) in acetic anhydride (20 mL) was stirred at 120° C. for 16 h. The reaction mixture was concentrated under vacuum. The residue was triturated with petroleum ether (100 mL) and the solid was filtered to afford 3-bromofuro[3,4-b]pyridine-5,7-dione (4.5 g, 87% yield) as a brown solid. LC-MS: m/z 228 [M+H]⁺.

### Step 2: 5-bromo-2-(isopropoxycarbonyl)nicotinic acid

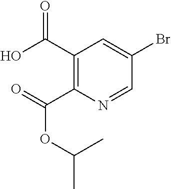 [SMILES: CC(C)OC(=O)c1ncc(Br)cc1C(=O)O]

A mixture of 3-bromofuro[3,4-b]pyridine-5,7-dione (4.4 g, 19.3 mmol) in isopropanol (100 mL) was stirred at 90° C. for 16 h. The reaction mixture was cooled to 25° C. The reaction mixture was concentrated under vacuum to afford 5-bromo-2-(isopropoxycarbonyl) nicotinic acid (4.4 g, 79% yield) as a brown solid. LC-MS: m/z 288 [M+H]⁺.

### Step 3: isopropyl 5-bromo-3-(hydroxymethyl)picolinate

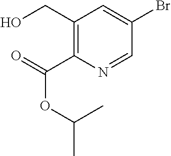

A solution of 5-bromo-2-(isopropoxycarbonyl) nicotinic acid (5.0 g, 17.4 mmol) in thionyl chloride (3.1 g, 26.0 mmol) was stirred at 40° C. for 3 h. The reaction mixture was concentrated under vacuum. The residue was dissolved in tetrahydrofuran (100 mL), and sodium borohydride (985 mg, 26.0 mmol) was added in batches at 0° C. The reaction mixture was stirred at 0° C. for 1 h. The reaction mixture was quenched with water (50 mL). The resulting solution was extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate as eluents to afford isopropyl 5-bromo-3-(hydroxymethyl)picolinate (2.5 g, 38% yield) as a white solid. LC-MS: m/z 274 [M+H]⁺.

### Step 4: isopropyl 5-bromo-3-formylpicolinate

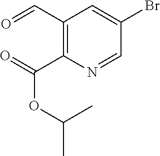

To a stirred solution of isopropyl 5-bromo-3-(hydroxymethyl)picolinate (2.5 g, 9.1 mmol) in dichloromethane (20 mL) was added Dess-Martin Periodinane (4.6 g, 10.9 mmol). The reaction mixture was stirred at 25° C. for 2 h. The reaction mixture was concentrated. The residue was purified by column chromatography on silica gel using 80% petroleum ether and 20% ethyl acetate as eluents to afford isopropyl 5-bromo-3-formylpicolinate (1.1 g, 44% yield) as a white solid. LC-MS: m/z 272 [M+H]⁺.

### Step 5: isopropyl 5-bromo-3-(difluoromethyl)picolinate

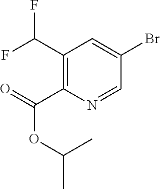 [SMILES: CC(C)OC(=O)c1ncc(Br)cc1C(F)F]

To a stirred solution of isopropyl 5-bromo-3-formylpicolinate (1.1 g, 4.0 mmol) in dichloromethane (20 mL) was added DAST (1.9 g, 12.1 mmol) dropwise at 0° C. The reaction mixture was stirred at 0° C. for 2 h. The reaction mixture was quenched with water (30 mL). The resulting solution was extracted with dichloromethane (3×30 mL). The combined organic layers were washed with brine (60 mL), dried over anhydrous sodium sulfate and concentrated under vacuum. The residue was purified by column chromatography on silica gel using 80% petroleum ether and 20% ethyl acetate as eluents to afford isopropyl 5-bromo-3-(difluoromethyl)picolinate (500 mg, 42% yield) as a yellow oil. ¹H NMR (400 MHz, Chloroform-d) δ: 8.87 (d, J=2.4, 1H), 8.28 (d, J=2.4, 1H), 7.45 (t, J=55.2 Hz, 1H), 5.31-5.38 (m, 1H), 1.44 (d, J=6.4 Hz, 6H). LC-MS: m/z 294 [M+H]⁺.

### Step 6: 5-bromo-3-(difluoromethyl)picolinic acid

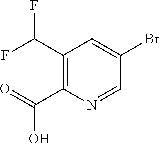 [SMILES: O=C(O)c1ncc(Br)cc1C(F)F]

To a stirred mixture of isopropyl 5-bromo-3-(difluoromethyl)picolinate (500 mg, 1.7 mmol) in tetrahydrofuran (5 mL) and water (5 mL) was added NaOH (748 mg, 18.7 mmol). The reaction mixture was stirred at 25° C. for 2 h. The pH was adjusted to 3 with HCl (1 M). The resulting solution was extracted with ethyl acetate (3×10 mL). The combined organic solution was dried over anhydrous sodium sulfate and concentrated under vacuum to afford 5-bromo-3-(difluoromethyl)picolinic acid (400 mg, 86% yield) as a white solid. LC-MS: m/z 252 [M+H]⁺.

### Step 7: 5-bromo-3-(difluoromethyl)-N,N-dimethylpicolinamide

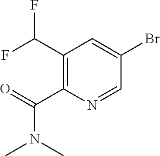

To a stirred solution of 5-bromo-3-(difluoromethyl)picolinic acid (300 mg, 1.2 mmol) in N,N-dimethylacetamide (10 mL) was added dimethylamine hydrochloride (194 mg, 2.4 mmol), EDCI (297 mg, 1.5 mmol), HOBt (209 mg, 1.5 mmol) and DIEA (461 mg, 3.6 mmol). The reaction mixture was stirred at 25° C. for 2 h. The reaction mixture was quenched with water (30 mL). The resulting solution was extracted with ethyl acetate (3×30 mL). The combined organic layers were washed with brine (50 mL), dried over anhydrous sodium sulfate and concentrated under vacuum. The residue was purified by column chromatography on silica gel using 60% petroleum ether and 40% ethyl acetate as eluents to afford 5-bromo-3-(difluoromethyl)-N,N-dimethylpicolinamide (200 mg, 60% yield) as a yellow oil. LC-MS: m/z 279 [M+H]⁺.

### Step 8: 3-(difluoromethyl)-5-((diphenylmethylene)amino)-N,N-dimethylpicolinamide

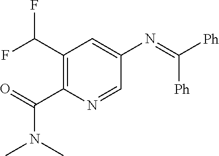 [SMILES: CN(C)C(=O)c1ncc(N=C(c2ccccc2)c2ccccc2)cc1C(F)F]

To a mixture of 5-bromo-3-(difluoromethyl)-N,N-dimethylpicolinamide (200 mg, 717 μmol) in dioxane (10 mL) was added diphenylmethanimine (260 mg, 1.4 mmol), Pd₂(dba)₃ (223 mg, 215 μmol), Xantphos (124 mg, 215 μmol) and Cs₂CO₃ (700 mg, 2.1 mmol) under nitrogen atmosphere. The resulting mixture was stirred at 110° C. for 16 h. The reaction mixture was concentrated under vacuum. The resulting mixture was diluted with water (10 mL) and extracted with ethyl acetate (3×10 mL). The combined organic layers were dried over anhydrous sodium sulfate and concentrated under vacuum. The residue was purified by column chromatography on silica gel using 75% petroleum ether and 25% ethyl acetate to afford 3-(difluoromethyl)-5-((diphenylmethylene)amino)-N,N-dimethylpicolinamide (200 mg, 74% yield) as a yellow solid. LC-MS: m/z 380 [M+H]⁺.

### Step 9: 5-amino-3-(difluoromethyl)-N,N-dimethylpicolinamide

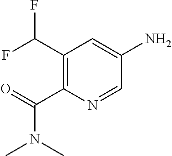

To a solution of 3-(difluoromethyl)-5-((diphenylmethylene)amino)-N,N-dimethylpicolinamide (200 mg, 527 μmol) in ethyl acetate (5 mL) was added HCl (1 mL, 1 M). The resulting mixture was stirred at 25° C. for 2 h. The pH was adjusted to 7-8 with saturated aqueous NaHCO₃ solution. The resulting solution was extracted with ethyl acetate (3×5 mL). The organic layers were combined, dried over anhydrous sodium sulfate and concentrated under vacuum. The residue was purified by column chromatography on silica gel using 90% dichloromethane and 10% methanol as eluent to afford 5-amino-3-(difluoromethyl)-N,N-dimethylpicolinamide (90 mg, 78% yield) as a yellow solid. ¹H NMR (300 MHz, Chloroform-d) S: 8.10 (d, J=2.7 Hz, 1H), 7.28 (d, J=2.7 Hz, 1H), 7.06 (t, J=55.8 Hz, 1H), 3.14 (s, 3H), 2.99 (s, 3H). LC-MS: m/z 216 [M+H]⁺.

### Step 10: (R)-2-chloro-N-(5-(difluoromethyl)-6-(dimethylcarbamoyl)pyridin-3-yl)-8-methyl-8-(trifluoromethyl)-7,8-dihydro-6H-pyrazolo[1,5-a]pyrrolo[2,3-e]pyrimidine-6-carboxamide

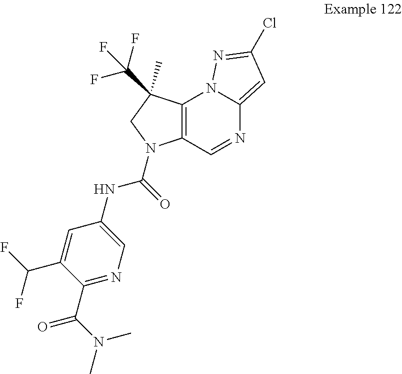 [SMILES: CN(C)C(=O)c1ncc(NC(=O)N2C[C@@](C)(C(F)(F)F)c3c2cnc2cc(Cl)nn32)cc1C(F)F]

To a stirred solution of 5-amino-3-(difluoromethyl)-N,N-dimethylpicolinamide (50 mg, 232 μmol) in tetrahydrofuran (1 mL) were added triphosgene (41 mg, 139 μmol) and TEA (35 mg, 348 μmol). The resulting mixture was stirred at 25° C. for 1 h and then filtered. The filtrate was added to a solution of Method M1 isomer 2 (77 mg, 279 μmol) in tetrahydrofuran (1 mL). To this solution were added N,N-dimethylpyridin-4-amine (57 mg, 465 μmol) and TEA (235 mg, 2.3 mmol). The mixture was stirred at 40° C. for 3 h. The mixture was concentrated under vacuum. The residue was purified by Prep-HPLC and the collected fractions were lyophilized to afford (R)-2-chloro-N-(5-(difluoromethyl)-6-(dimethylcarbamoyl)pyridin-3-yl)-8-methyl-8-(trifluoromethyl)-7,8-dihydro-6H-pyrazolo[1,5-a]pyrrolo[2,3-e]pyrimidine-6-carboxamide (18 mg, 15% yield) as a white solid. The enantiomer of Example 122 can be prepared analogously using Method M1 isomer 1.

Example 122: ¹H NMR (300 MHz, DMSO-d₆) δ: 9.54 (s, 1H), 9.33 (s, 1H), 8.91 (d, J=2.1 Hz, 1H), 8.36 (d, J=2.1 Hz, 1H), 7.10 (t, J=54.9 Hz, 1H), 7.05 (s, 1H), 4.83 (d, J=11.7 Hz, 1H), 4.27 (d, J=11.7 Hz, 1H), 3.01 (s, 3H), 2.83 (s, 3H), 1.96 (s, 3H). LC-MS: m/z 518 [M+H]⁺.

### Method J4

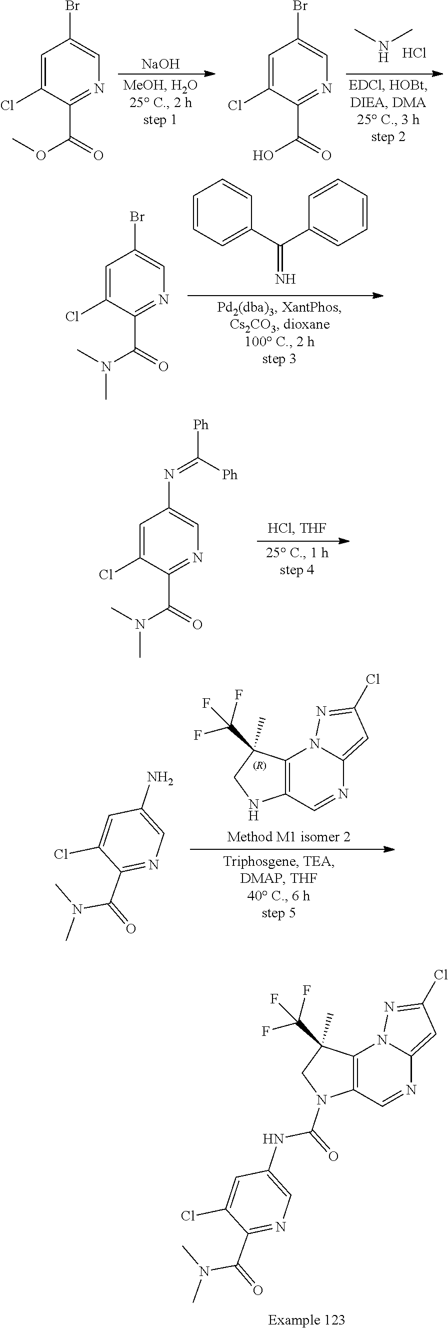

### Example 123: (R)-2-chloro-N-(5-chloro-6-(dimethylcarbamoyl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 5-bromo-3-chloropicolinic acid

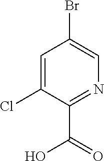 [SMILES: O=C(O)c1ncc(Br)cc1Cl]

To a stirred solution of methyl 5-bromo-3-chloropicolinate (5 g, 20.0 mmol) in methanol (40 mL) and water (20 mL) was added sodium hydroxide (1.6 g, 39.9 mmol). The reaction mixture was stirred at 25° C. for 2 h. The pH was adjusted to 3 with HCl (1 M). The resulting solution was extracted with ethyl acetate (3×50 mL). The combined organic solution was dried over anhydrous sodium sulfate and concentrated under vacuum to afford 5-bromo-3-chloropicolinic acid (3.7 g, 77% yield) as a yellow oil. LC-MS: m/z 236 [M+H]⁺.

### Step 2: 5-bromo-3-chloro-N,N-dimethylpicolinamide

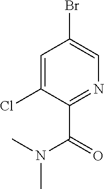

To a stirred solution of 5-bromo-3-chloropicolinic acid (3.7 g, 15.5 mmol) in N,N-dimethylacetamide (40 mL) was added dimethylamine hydrochloride (1.3 g, 15.6 mmol), EDCI (3.9 g, 20.3 mmol), HOBt (2.7 g, 20.3 mmol) and DIEA (6.0 g, 46.7 mmol). The reaction mixture was stirred at 25° C. for 3 h. The reaction mixture was quenched with water (100 mL). The resulting solution was extracted with ethyl acetate (3×100 mL). The combined organic layers were washed with brine (200 mL), dried over anhydrous sodium sulfate and concentrated under vacuum. The residue was purified by column chromatography on silica gel using 50% petroleum ether and 50% ethyl acetate as eluents to afford 5-bromo-3-chloro-N,N-dimethylpicolinamide (3.4 g, 70% yield) as a white solid. LC-MS: m/z 263 [M+H]⁺.

### Step: 3: 3-chloro-5-((diphenylmethylene)amino)-N,N-dimethylpicolinamide

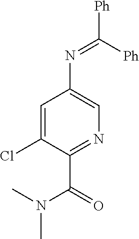 [SMILES: CN(C)C(=O)c1ncc(N=C(c2ccccc2)c2ccccc2)cc1Cl]

To a mixture of 5-bromo-3-chloro-N,N-dimethylpicolinamide (2.0 g, 7.6 mmol) in dioxane (30 mL) was added diphenylmethanimine (1.4 g, 7.5 mmol), Pd₂(dba)₃ (780 mg, 753.5 μmol), Xantphos (440 mg, 760 μmol) and Cs₂CO₃ (7.4 g, 22.8 mmol) under nitrogen atmosphere. The resulting mixture was stirred at 100° C. for 2 h. The reaction mixture was cooled to 25° C. and concentrated under vacuum. The resulting mixture was diluted with water (50 mL), and extracted with ethyl acetate (3×50 mL). The combined organic layers were dried over anhydrous sodium sulfate and concentrated under vacuum. The residue was purified by column chromatography on silica gel using 90% petroleum ether and 10% ethyl acetate to afford 3-chloro-5-((diphenylmethylene)amino)-N,N-dimethylpicolinamide (1.7 g, 61% yield) as a yellow oil. LC-MS: m/z 364 [M+H]⁺.

### Step 4: 5-amino-3-chloro-N,N-dimethylpicolinamide

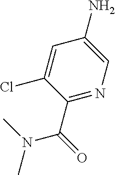

To a solution of 3-chloro-5-((diphenylmethylene)amino)-N,N-dimethylpicolinamide (700 mg, 1.9 mmol) in tetrahydrofuran (10 mL) was added HCl (4 mL, 1 M). The resulting mixture was stirred at 25° C. for 1 h. The pH was adjusted to 7-8 with saturated aqueous NaHCO₃ solution. The resulting solution was extracted with ethyl acetate (3×20 mL). The organic layers were combined, dried over anhydrous sodium sulfate and concentrated under vacuum. The residue was purified by column chromatography on silica gel using 90% dichloromethane and 10% methanol as eluents to afford 5-amino-3-chloro-N,N-dimethylpicolinamide (252 mg, 65% yield) as a white solid. LC-MS: m/z 200 [M+H]⁺.

### Step 5: (R)-2-chloro-N-(5-chloro-6-(dimethylcarbamoyl)pyridin-3-yl)-8-methyl-8-(trifluoromethyl)-7,8-dihydro-6H-pyrazolo[1,5-a]pyrrolo[2,3-e]pyrimidine-6-carboxamide

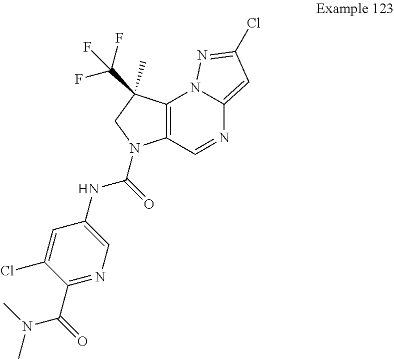 [SMILES: CN(C)C(=O)c1ncc(NC(=O)N2C[C@@](C)(C(F)(F)F)c3c2cnc2cc(Cl)nn32)cc1Cl]

To a stirred solution of Method M1 isomer 2 (100 mg, 361 μmol) in tetrahydrofuran (4 mL) were added triphosgene (64 mg, 217 μmol) and TEA (55 mg, 542 μmol). The resulting mixture was stirred at 25° C. for 1 h and then filtered. The filtrate was added to a solution of 5-amino-3-chloro-N,N-dimethylpicolinamide (108 mg, 542 μmol) in tetrahydrofuran (2 mL). To this solution were added N,N-dimethylpyridin-4-amine (88 mg, 723 μmol) and TEA (366 mg, 3.6 mmol). The mixture was stirred at 40° C. for 6 h. The mixture was concentrated under vacuum. The residue was purified by Prep-HPLC and the collected fractions were lyophilized to afford (R)-2-chloro-N-(5-chloro-6-(dimethylcarbamoyl)pyridin-3-yl)-8-methyl-8-(trifluoromethyl)-7,8-dihydro-6H-pyrazolo[1,5-a]pyrrolo[2,3-e]pyrimidine-6-carboxamide (13.7 mg, 7% yield) as a white solid. The enantiomer of Example 123 can be prepared analogously using Method M1 isomer 1.

Example 123: ¹H NMR (400 MHz, DMSO-d₆) δ: 9.52 (s, 1H), 9.34 (s, 1H), 8.72 (s, 1H), 8.27 (s, 1H), 7.07 (s, 1H), 4.83 (d, J=11.6 Hz, 1H), 4.27 (d, J=11.6 Hz, 1H), 3.02 (s, 3H), 2.78 (s, 3H), 1.98 (s, 3H). LC-MS: m/z 502 [M+H]⁺.

### Method K4

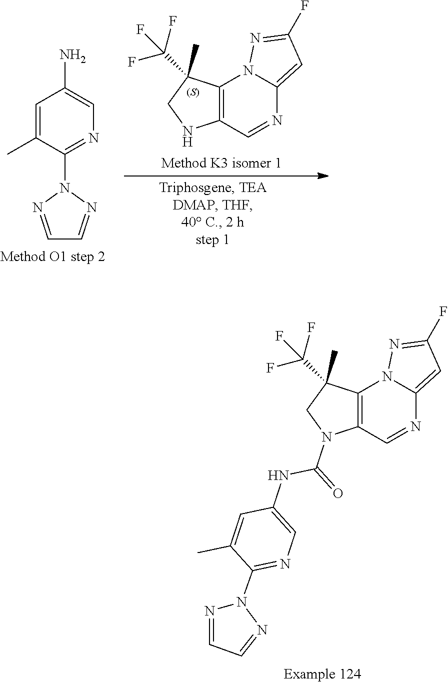

### Example 124: (S)-2-fluoro-8-methyl-N-(5-methyl-6-(2H-1,2,3-triazol-2-yl)pyridin-3-yl)-8-(trifluoromethyl)-7,8-dihydro-6H-pyrazolo[1,5-a]pyrrolo[2,3-e]pyrimidine-6-carboxamide

### Step 1: (S)-2-fluoro-8-methyl-N-(5-methyl-6-(2H-1,2,3-triazol-2-yl)pyridin-3-yl)-8-(trifluoromethyl)-7,8-dihydro-6H-pyrazolo[1,5-a]pyrrolo[2,3-e]pyrimidine-6-carboxamide

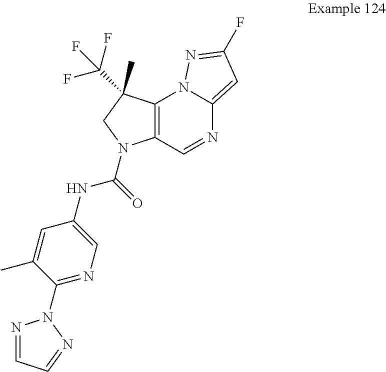 [SMILES: Cc1cc(NC(=O)N2C[C@](C)(C(F)(F)F)c3c2cnc2cc(F)nn32)cnc1-n1nccn1]

To a stirred solution of (S)-2-fluoro-8-methyl-8-(trifluoromethyl)-7,8-dihydro-6H-pyrazolo[1,5-a]pyrrolo[2,3-e]pyrimidine (Method K3 isomer 1; 30 mg, 115.3 μmol) in tetrahydrofuran (1 mL) were added triphosgene (20 mg, 69.1 μmol) and TEA (17 mg, 172.9 μmol). The resulting mixture was stirred at 25° C. for 0.5 h and then filtered. The filtrate was added to a solution of 5-methyl-6-(2H-1,2,3-triazol-2-yl)pyridin-3-amine (Method O1 step 2; 35 mg, 172.9 μmol) in tetrahydrofuran (1 mL). To this solution were added N,N-dimethylpyridin-4-amine (21 mg, 173.0 μmol) and TEA (117 mg, 1.2 mmol). The mixture was stirred at 40° C. for 2 h. The mixture was concentrated under vacuum. The residue was purified by Prep-HPLC and the collected fractions were lyophilized to afford (S)-2-fluoro-8-methyl-N-(5-methyl-6-(2H-1,2,3-triazol-2-yl)pyridin-3-yl)-8-(trifluoromethyl)-7,8-dihydro-6H-pyrazolo[1,5-a]pyrrolo[2,3-e]pyrimidine-6-carboxamide (16.6 mg, 31% yield) as a yellow solid.

Example 124: ¹H NMR (400 MHz, Chloroform-d) δ: 9.41 (s, 1H), 8.35 (s, 1H), 8.27 (s, 1H), 7.89 (s, 2H), 6.94 (s, 1H), 6.34 (d, J=5.2 Hz, 1H), 4.61 (d, J=10.4 Hz, 1H), 4.06 (d, J=10.4 Hz, 1H), 2.50 (s, 3H), 2.04 (s, 3H). LC-MS: m/z 462 [M+H]⁺.

### Method L4

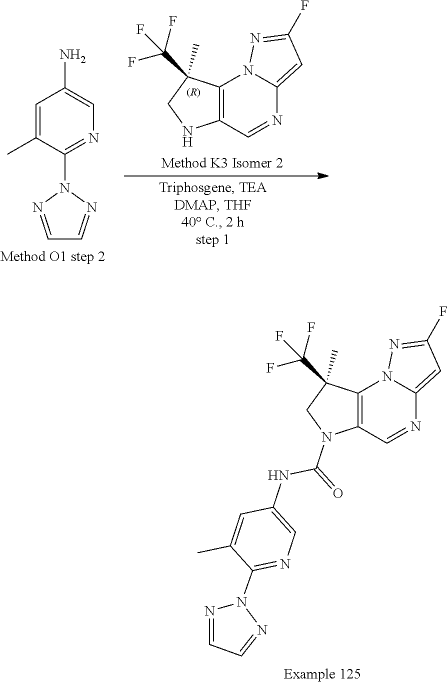

### Example 125: (R)-2-fluoro-8-methyl-N-(5-methyl-6-(2H-1,2,3-triazol-2-yl)pyridin-3-yl)-8-(trifluoromethyl)-7,8-dihydro-6H-pyrazolo[1,5-a]pyrrolo[2,3-e]pyrimidine-6-carboxamide

### Step 1: (R)-2-fluoro-8-methyl-N-(5-methyl-6-(2H-1,2,3-triazol-2-yl)pyridin-3-yl)-8-(trifluoromethyl)-7,8-dihydro-6H-pyrazolo[1,5-a]pyrrolo[2,3-e]pyrimidine-6-carboxamide

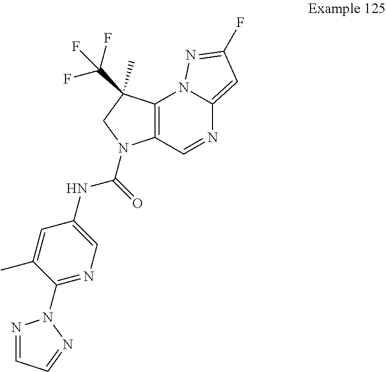 [SMILES: Cc1cc(NC(=O)N2C[C@@](C)(C(F)(F)F)c3c2cnc2cc(F)nn32)cnc1-n1nccn1]

To a stirred solution of (R)-2-fluoro-8-methyl-8-(trifluoromethyl)-7,8-dihydro-6H-pyrazolo[1,5-a]pyrrolo[2,3-e]pyrimidine (Method K3 isomer 2; 30 mg, 115.3 μmol) in tetrahydrofuran (1 mL) were added triphosgene (20 mg, 69.1 μmol) and TEA (17 mg, 172.9 μmol). The resulting mixture was stirred at 25° C. for 0.5 h and then filtered. The filtrate was added to a solution of 5-methyl-6-(2H-1,2,3-triazol-2-yl)pyridin-3-amine (Method O1 step 2; 62 mg, 230.6 μmol) in tetrahydrofuran (1 mL). To this solution were added N,N-dimethylpyridin-4-amine (21 mg, 173.0 μmol) and TEA (117 mg, 1.2 mmol). The mixture was stirred at 40° C. for 2 h. The mixture was concentrated under vacuum. The residue was purified by Prep-HPLC and the collected fractions were lyophilized to afford (R)-2-fluoro-8-methyl-N-(5-methyl-6-(2H-1,2,3-triazol-2-yl)pyridin-3-yl)-8-(trifluoromethyl)-7,8-dihydro-6H-pyrazolo[1,5-a]pyrrolo[2,3-e]pyrimidine-6-carboxamide (10.9 mg, 21% yield) as a white solid.

Example 125: ¹H NMR (400 MHz, Chloroform-d) δ: 9.41 (s, 1H), 8.33 (s, 1H), 8.26 (s, 1H), 7.90 (s, 2H), 6.71 (s, 1H), 6.34 (d, J=5.2 Hz, 1H), 4.59 (d, J=10.4 Hz, 1H), 4.06 (d, J=10.4 Hz, 1H), 2.50 (s, 3H), 2.05 (s, 3H). LC-MS: m/z 462 [M+H]⁺.

### Method M4

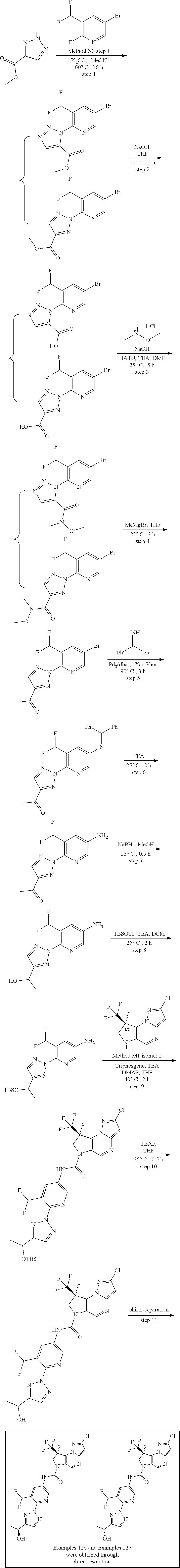

### Example 126 and 127: Single enantiomers obtained from a racemic mixture containing (R)-2-chloro-N-(5-(difluoromethyl)-6-(4-((S)-1-hydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(5-(difluoromethyl)-6-(4-((R)-1-hydroxyethyl)-2H-1,2,3-triazol-2-yl)pyridin-3-yl)-8-methyl-(trifluoromethyl)-7,8-dihydro-6H-pyrazolo[1,5-a]pyrrolo[2,3-e]pyrimidine-6-carboxamide

### Step 1: Mixture of methyl 2-(5-bromo-3-(difluoromethyl)pyridin-2-yl)-2H-1,2,3-triazole-4-carboxylate and methyl 1-(5-bromo-3-(difluoromethyl)pyridin-2-yl)-1H-1,2,3-triazole-5-carboxylate

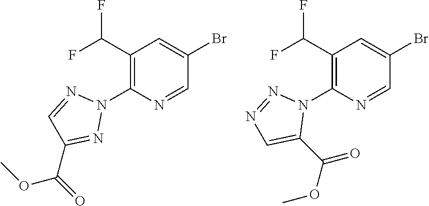 [SMILES: COC(=O)c1cnn(-c2ncc(Br)cc2C(F)F)n1 ; COC(=O)c1cnnn1-c1ncc(Br)cc1C(F)F]

To a solution of methyl 2H-triazole-4-carboxylate (8.0 g, 62.9 mmol) in acetonitrile (130 mL) were added K₂CO₃ (26.1 g, 188.8 mmol) and 5-bromo-3-(difluoromethyl)-2-fluoro-pyridine (Method X3, step 1; 15.6 g, 69.2 mmol). The resulting mixture was stirred at 60° C. for 16 h. After cooled to 25° C., the reaction mixture was quenched with water (100 mL). The resulting solution was extracted with ethyl acetate (3×100 mL). The combined organic layers were dried over anhydrous sodium sulfate and concentrated under vacuum. The residue was purified by column chromatography on silica gel using 30% petroleum ether and 70% ethyl acetate as eluent to afford a mixture of 2-(5-bromo-3-(difluoromethyl)pyridin-2-yl)-2H-1,2,3-triazole-4-carboxylate and methyl 1-(5-bromo-3-(difluoromethyl)pyridin-2-yl)-1H-1,2,3-triazole-5-carboxylate (7.5 g, 35% yield) as a white solid. LC-MS: m/z 333 [M+H]⁺.

### Step 2: Mixture of 2-(5-bromo-3-(difluoromethyl)pyridin-2-yl)-2H-1,2,3-triazole-4-carboxylic acid and 1-(5-bromo-3-(difluoromethyl)pyridin-2-yl)-1H-1,2,3-triazole-5-carboxylic acid

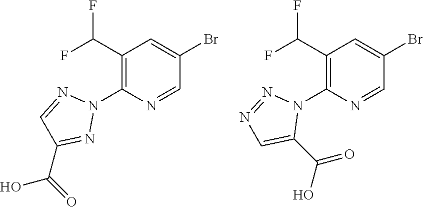 [SMILES: O=C(O)c1cnn(-c2ncc(Br)cc2C(F)F)n1 ; O=C(O)c1cnnn1-c1ncc(Br)cc1C(F)F]

A mixture of 2-(5-bromo-3-(difluoromethyl)pyridin-2-yl)-2H-1,2,3-triazole-4-carboxylate and methyl 1-(5-bromo-3-(difluoromethyl)pyridin-2-yl)-1H-1,2,3-triazole-5-carboxylate (2.0 g, 6.0 mmol) in tetrahydrofuran (40 mL) was added NaOH (480 mg, 12.0 mmol) in water (8 mL). The resulting solution was stirred at 25° C. for 2 h. The pH was adjusted to 3-4 with HCl (1M). The mixture was concentrated to remove tetrahydrofuran. The resulting mixture was extracted with ethyl acetate (3×50 mL). The combined organic layers were dried over anhydrous sodium sulfate and concentrated under vacuum to afford a mixture of 2-(5-bromo-3-(difluoromethyl)pyridin-2-yl)-2H-1,2,3-triazole-4-carboxylic acid and 1-(5-bromo-3-(difluoromethyl)pyridin-2-yl)-1H-1,2,3-triazole-5-carboxylic acid (1.2 g, 62% yield) as a white solid. LC-MS: m/z 319 [M+H]⁺.

### Step 3: Mixture of 2-(5-bromo-3-(difluoromethyl)pyridin-2-yl)-N-methoxy-N-methyl-2H-1,2,3-triazole-4-carboxamide and 1-(5-bromo-3-(difluoromethyl)pyridin-2-yl)-N-methoxy-N-methyl-1H-1,2,3-triazole-5-carboxamide

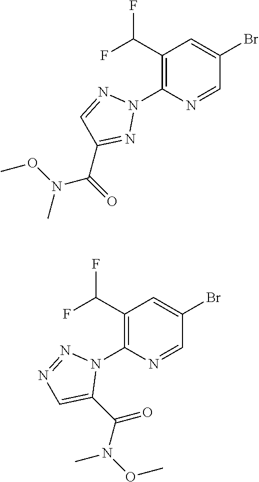 [SMILES: CON(C)C(=O)c1cnn(-c2ncc(Br)cc2C(F)F)n1 ; CON(C)C(=O)c1cnnn1-c1ncc(Br)cc1C(F)F]

A mixture of2-(5-bromo-3-(difluoromethyl)pyridin-2-yl)-2H-1,2,3-triazole-4-carboxylic acid and 1-(5-bromo-3-(difluoromethyl)pyridin-2-yl)-1H-1,2,3-triazole-5-carboxylic acid (1.5 g, 4.7 mmol) and N,O-dimethylhydroxylamine; hydrochloride (700 mg, 7 mmol) in N,N-Dimethylformamide (10 mL) was added HATU (2.7 g, 7.1 mmol) and TEA (1.4 g, 14.1 mmol). The resulting solution was stirred at 25° C. for 5 h. The reaction mixture was quenched with water (100 mL). The resulting solution was extracted with ethyl acetate (3×100 mL). The combined organic layers were dried over anhydrous sodium sulfate and concentrated under vacuum. The residue was purified by column chromatography on silica gel using 50% petroleum ether and 50% ethyl acetate as eluents to afford a mixture of 2-(5-bromo-3-(difluoromethyl)pyridin-2-yl)-N-methoxy-N-methyl-2H-1,2,3-triazole-4-carboxamide and 1-(5-bromo-3-(difluoromethyl)pyridin-2-yl)-N-methoxy-N-methyl-1H-1,2,3-triazole-5-carboxamide (1.3 g, 76% yield) as a white solid. LC-MS: m/z 362 [M+H]⁺.

### Step 4: 1-(2-(5-bromo-3-(difluoromethyl)pyridin-2-yl)-2H-1,2,3-triazol-4-yl)ethan-1-one

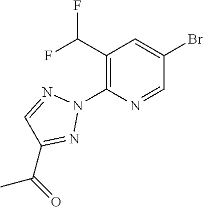 [SMILES: CC(=O)c1cnn(-c2ncc(Br)cc2C(F)F)n1]

A mixture of 2-(5-bromo-3-(difluoromethyl)pyridin-2-yl)-N-methoxy-N-methyl-2H-1,2,3-triazole-4-carboxamide and 1-(5-bromo-3-(difluoromethyl)pyridin-2-yl)-N-methoxy-N-methyl-1H-1,2,3-triazole-5-carboxamide (7.0 g, 19.3 mmol) in tetrahydrofuran (140 mL) was added methylmagnesium bromide (58 mL, 58.0 mmol, 1 M in THF) dropwise at −20° C. under nitrogen. The resulting solution was stirred at −20° C. for 3 h. The reaction mixture was quenched with water (200 mL). The resulting solution was extracted with ethyl acetate (3×200 mL). The combined organic layers were dried over anhydrous sodium sulfate and concentrated under vacuum. The residue was purified by column chromatography on silica gel using 60% petroleum ether and 40% ethyl acetate as eluents to afford 1-(2-(5-bromo-3-(difluoromethyl)pyridin-2-yl)-2H-1,2,3-triazol-4-yl)ethan-1-one (1.8 g, 29% yield) as a white solid. ¹H NMR (300 MHz, Chloroform-d) δ: 8.84 (d, J=2.1 Hz, 1H) 8.43 (d, J=2.1 Hz, 1H), 8.37 (s, 1H), 7.67 (t, J=54.3 Hz, 1H), 2.76 (s, 3H). LC-MS: m/z 317 [M+H]⁺.

### Step 5: 1-(2-(3-(difluoromethyl)-5-((diphenylmethylene)amino)pyridin-2-yl)-2H-1,2,3-triazol-4-yl)ethan-1-one

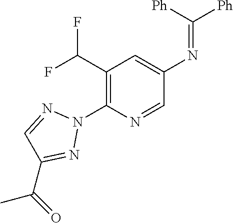 [SMILES: CC(=O)c1cnn(-c2ncc(N=C(c3ccccc3)c3ccccc3)cc2C(F)F)n1]

To a stirred solution of 1-(2-(5-bromo-3-(difluoromethyl)pyridin-2-yl)-2H-1,2,3-triazol-4-yl)ethan-1-one (1.8 g, 5.7 mmol) and diphenylmethanimine (1.1 g, 6.2 mmol) in dioxane (5 mL) were added XantPhos (246 mg, 425.5 μmol), Pd₂(dba)₃ (259 mg, 283.8 μmol) and Cs₂CO₃ (4.6 g, 14.1 mmol). The resulting mixture was stirred at 90° C. for 3 h. After cooled to 25° C., the reaction mixture was quenched with water (30 mL). The resulting solution was extracted with ethyl acetate (3×30 mL). The combined organic layers were dried over anhydrous sodium sulfate and concentrated under vacuum. The residue was purified by column chromatography on silica gel using 50% petroleum ether and 50% ethyl acetate as eluents to afford 1-(2-(3-(difluoromethyl)-5-((diphenylmethylene)amino)pyridin-2-yl)-2H-1,2,3-triazol-4-yl)ethan-1-one (850 mg, 35% yield) as a white solid. LC-MS: m/z 418 [M+H]⁺.

### Step 6: 1-(2-(5-amino-3-(difluoromethyl)pyridin-2-yl)-2H-1,2,3-triazol-4-yl)ethan-1-one

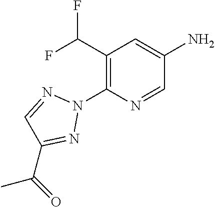 [SMILES: CC(=O)c1cnn(-c2ncc(N)cc2C(F)F)n1]

A solution of 1-(2-(3-(difluoromethyl)-5-((diphenylmethylene)amino)pyridin-2-yl)-2H-1,2,3-triazol-4-yl)ethan-1-one (890 mg, 2.1 mmol) in TFA (20 mL) was stirred at 25° C. for 2 h. The solution was concentrated under vacuum. The pH was adjusted to 7 with saturated aqueous NaHCO₃ solution. The resulting solution was extracted with ethyl acetate (3×20 mL). The combined organic layers were dried over anhydrous sodium sulfate and concentrated under vacuum. The residue was purified by column chromatography on silica gel using 50% petroleum ether and 50% ethyl acetate as eluents to afford 1-(2-(5-amino-3-(difluoromethyl)pyridin-2-yl)-2H-1,2,3-triazol-4-yl)ethan-1-one (320 mg, 59% yield) as a yellow solid. ¹H NMR (300 MHz, Chloroform-d) δ: 8.77 (s, 1H), 8.06 (d, J=2.4 Hz, 1H), 7.49 (d, J=2.4 Hz, 1H), 7.45 (t, J=54.6 Hz, 1H), 2.78 (s, 3H). LC-MS: m/z 254 [M+H]⁺.

### Step 7: 1-(2-(5-amino-3-(difluoromethyl)pyridin-2-yl)-2H-1,2,3-triazol-4-yl)ethan-1-ol

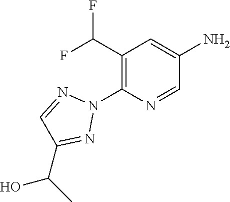 [SMILES: CC(O)c1cnn(-c2ncc(N)cc2C(F)F)n1]

To a stirred mixture of 1-[2-[5-amino-3-(difluoromethyl)-2-pyridyl]triazol-4-yl]ethanone (150 mg, 592.4 μmol) in methanol (10 mL) was added NaBH₄ (27 mg, 710.8 μmol) at 0° C. The reaction mixture was stirred at 25° C. for 0.5 h. The reaction mixture was concentrated under vacuum. The residue was purified by Prep-TLC using 40% petroleum ether and 60% ethyl acetate as eluents to afford 1-(2-(5-amino-3-(difluoromethyl)pyridin-2-yl)-2H-1,2,3-triazol-4-yl)ethan-1-ol (120 mg, 79% yield) as a colorless oil. ¹H NMR (300 MHz, Chloroform-d) δ: 8.13 (d, J=3.0 Hz, 1H), 7.86 (s, 1H), 7.46 (d, J=2.7 Hz, 1H), 7.33 (t, J=54.9 Hz, 1H), 5.21 (q, J=6.6 Hz, 1H), 1.67 (d, J=6.6 Hz, 3H). LC-MS: m/z 256 [M+H]⁺.

### Step 8: 6-(4-(1-((tert-butyldimethylsilyl)oxy)ethyl)-2H-1,2,3-triazol-2-yl)-5-(difluoromethyl)pyridin-3-amine

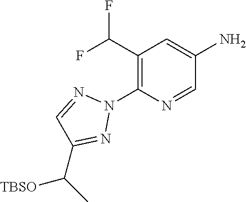 [SMILES: CC(O[Si](C)(C)C(C)(C)C)c1cnn(-c2ncc(N)cc2C(F)F)n1]

To a stirred mixture of 1-(2-(5-amino-3-(difluoromethyl)pyridin-2-yl)-2H-1,2,3-triazol-4-yl)ethan-1-ol (120 mg, 470.2 μmol) in dichloromethane (10 mL) were added TEA (142.7 mg, 1.4 mmol) and tert-butyldimethylsilyl trifluoromethanesulfonate (248.5 mg, 940.3 μmol) at 25° C. The reaction mixture was stirred at 25° C. for 2 h. The reaction mixture was concentrated under vacuum. The residue was purified by Prep-TLC using 60% petroleum ether and 40% ethyl acetate as eluents to afford 6-(4-(1-((tert-butyldimethylsilyl)oxy)ethyl)-2H-1,2,3-triazol-2-yl)-5-(difluoromethyl)pyridin-3-amine (160 mg, 92% yield) as a light-yellow solid. ¹H NMR (300 MHz, Chloroform-d) δ: 8.11 (d, J=3.0 Hz, 1H), 7.81 (s, 1H), 7.43 (d, J=3.0 Hz, 1H), 7.32 (t, J=54.9 Hz, 1H), 5.17 (q, J=6.6 Hz, 1H), 1.55 (d, J=6.6 Hz, 3H), 0.92 (s, 9H), 0.11 (s, 3H), 0.05 (s, 3H). LC-MS: m/z 370 [M+H]⁺.

### Step 9: (8R)—N-(6-(4-(1-((tert-butyldimethylsilyl)oxy)ethyl)-2H-1,2,3-triazol-2-yl)-5-(difluoromethyl)pyridin-3-yl)-2-chloro-8-methyl-8-(trifluoromethyl)-7,8-dihydro-6H-pyrazolo[1,5-a]pyrrolo[2,3-e]pyrimidine-6-carboxamide

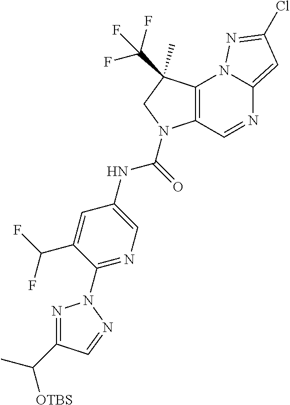 [SMILES: CC(O[Si](C)(C)C(C)(C)C)c1cnn(-c2ncc(NC(=O)N3C[C@@](C)(C(F)(F)F)c4c3cnc3cc(Cl)nn43)cc2C(F)F)n1]

To a stirred mixture of 6-(4-(1-((tert-butyldimethylsilyl)oxy)ethyl)-2H-1,2,3-triazol-2-yl)-5-(difluoromethyl)pyridin-3-amine (150 mg, 405.9 μmol) in tetrahydrofuran (5 mL) were added triphosgene (72. mg, 243.5 μmol) and TEA (62 mg, 608.9 μmol) at 25° C. The resulting mixture was stirred at 25° C. for 0.5 h and then filtered. The resulting filtrate was added to a solution of Method M1 isomer 2 (67 mg, 243.5 μmol) in tetrahydrofuran (1 mL). To this solution were added TEA (411 mg, 4.1 mmol) and N,N-Dimethylpyridin-4-amine (99.20 mg, 811.94 μmol) The mixture was stirred at 40° C. for 2 h. The reaction mixture was concentrated under vacuum. The residue was purified by Prep-TLC using 70% petroleum ether and 30% ethyl acetate as eluent to afford (8R)—N-(6-(4-(1-((tert-butyldimethylsilyl)oxy)ethyl)-2H-1,2,3-triazol-2-yl)-5-(difluoromethyl)pyridin-3-yl)-2-chloro-8-methyl-8-(trifluoromethyl)-7,8-dihydro-6H-pyrazolo[1,5-a]pyrrolo[2,3-e]pyrimidine-6-carboxamide (110 mg, 40% yield) as a white solid. ¹H NMR (400 MHz, DMSO-d₆) δ: 9.66 (br, 1H), 9.35 (s, 1H), 8.93 (d, J=2.4 Hz, 1H), 8.56 (d, J=2.4 Hz, 1H), 8.06 (s, 1H), 7.36 (t, J=54.4 Hz, 1H), 7.06 (s, 1H), 5.16 (q, J=6.4 Hz, 1H), 4.85 (d, J=11.2 Hz, 1H), 4.30 (d, J=11.2 Hz, 1H), 1.97 (s, 3H), 1.50 (d, J=6.4 Hz, 3H), 0.87 (s, 9H), 0.10 (s, 3H), 0.04 (s, 3H). LC-MS: m/z 672 [M+H]⁺.

### Step 10: (8R)-2-chloro-N-(5-(difluoromethyl)-6-(4-(1-hydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

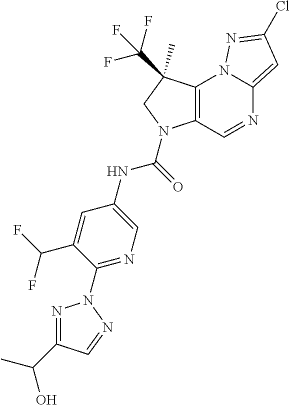 [SMILES: CC(O)c1cnn(-c2ncc(NC(=O)N3C[C@@](C)(C(F)(F)F)c4c3cnc3cc(Cl)nn43)cc2C(F)F)n1]

To a stirred mixture of (8R)—N-(6-(4-(1-((tert-butyldimethylsilyl)oxy)ethyl)-2H-1,2,3-triazol-2-yl)-5-(difluoromethyl)pyridin-3-yl)-2-chloro-8-methyl-8-(trifluoromethyl)-7,8-dihydro-6H-pyrazolo[1,5-a]pyrrolo[2,3-e]pyrimidine-6-carboxamide (110 mg, 163.6 μmol) in tetrahydrofuran (5 mL) was added TBAF (1 M in tetrahydrofuran, 1 mL) at 25° C. The reaction mixture was stirred at 25° C. for 0.5 h. The reaction mixture was concentrated. The residue was purified by Prep-TLC using 50% petroleum ether and 50% ethyl acetate as eluents to afford 70 mg of crude product. The crude product was submitted to Prep-HPLC purification and the collected fractions were lyophilized to afford (8R)-2-chloro-N-(5-(difluoromethyl)-6-(4-(1-hydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide (38 mg, 41% yield) as an off-white solid. ¹H NMR (300 MHz, DMSO-d₆) δ: 9.67 (br, 1H), 9.38 (s, 1H), 8.96 (s, 1H), 8.58 (s, 1H), 8.09 (s, 1H), 7.42 (t, J=54.6 Hz, 1H), 7.08 (s, 1H), 5.56 (d, J=5.1 Hz, 1H), 4.93-5.03 (m, 1H), 4.88 (d, J=11.4 Hz, 1H), 4.32 (d, J=11.4 Hz, 1H), 2.00 (s, 3H), 1.49 (d, J=6.6 Hz, 3H). LC-MS: m/z 558 [M+H]⁺.

### Step 11: Separation of enantiomers to obtain (R)-2-chloro-N-(5-(difluoromethyl)-6-(4-((R)-1-hydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(5-(difluoromethyl)-6-(4-((S)-1-hydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

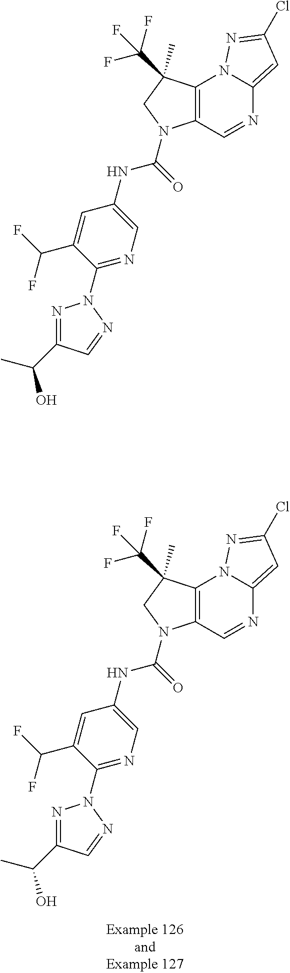 [SMILES: C[C@H](O)c1cnn(-c2ncc(NC(=O)N3C[C@@](C)(C(F)(F)F)c4c3cnc3cc(Cl)nn43)cc2C(F)F)n1 ; C[C@@H](O)c1cnn(-c2ncc(NC(=O)N3C[C@@](C)(C(F)(F)F)c4c3cnc3cc(Cl)nn43)cc2C(F)F)n1]

(8R)-2-chloro-N-(5-(difluoromethyl)-6-(4-(1-hydroxyethyl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide (35 mg, 62.7 μmol) was submitted to chiral HPLC purification Column: CHIRAL ART Cellulose-SB, 3×25 cm, 5 um; Mobile Phase A: Hex:DCM=3:1(0.5% 2M NH₃-MeOH)—HPLC, Mobile Phase B: EtOH—HPLC; Flow rate: 40 mL/min; Gradient: 7 B to 7 B in 37 min; 220/254 nm; RT1: 31.2; RT2: 34.3; Injection Volume: 0.4 ml; Number Of Runs: 7. The first eluting isomer was concentrated and lyophilized to afford Example 126 (9.0 mg, 26% yield) as a white solid. The second eluting isomer was concentrated and lyophilized to Example 127 (6.9 mg, 20% yield) as a white solid. The corresponding stereoisomers of Examples 126 and 127 can be prepared analogously using Method M1 isomer 1. Examples 126 and 127 are diastereomers, wherein the stereocenter attached to the trifluoromethyl is absolute and the carbinol stereocenter is relative (i.e., the carbinol stereocenter in one of Examples 126 and 127 is (S), and the carbinol stereocenter in the other of Examples 126 and 127 is (R)).

Example 126: ¹H NMR (400 MHz, DMSO-d₆) δ: 9.67 (s, 1H), 9.37 (s, 1H), 8.94 (d, J=2.4 Hz, 1H), 8.57 (d, J=2.4 Hz, 1H), 8.08 (s, 1H), 7.41 (t, J=54.4 Hz, 1H), 7.08 (s, 1H), 5.55 (d, J=4.8 Hz, 1H), 4.93-5.00 (m, 1H), 4.86 (d, J=11.6 Hz, 1H), 4.31 (d, J=11.6 Hz, 1H), 1.99 (s, 3H), 1.47 (d, J=6.4 Hz, 3H). LC-MS: m/z 558 [M+H]⁺.

Example 127: ¹H NMR (400 MHz, DMSO-d₆) δ: 9.65 (s, 1H), 9.36 (s, 1H), 8.93 (d, J=2.4 Hz, 1H), 8.56 (d, J=2.4 Hz, 1H), 8.07 (s, 1H), 7.39 (t, J=54.4 Hz, 1H), 7.06 (s, 1H), 5.54 (d, J=5.2 Hz, 1H), 4.89-5.00 (m, 1H), 4.85 (d, J=11.2 Hz, 1H), 4.30 (d, J=11.2 Hz, 1H), 1.97 (s, 3H), 1.47 (d, J=6.4 Hz, 3H). LC-MS: m/z 558 [M+H]⁺.

### Method N4

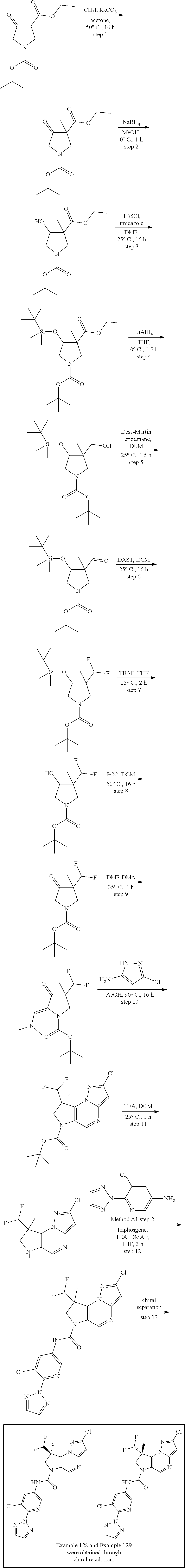

### Examples 128 and 129: Single enantiomers obtained from a racemic mixture containing (R)-2-chloro-N-(5-chlor H-1,2,3-triazol-2-yl)-pyridin-3-yl)-8-(difluoromethyl)-8-methyl-7,8-dihydro-6H-pyrazolo[1,5-a]pyrrolo[2,3-e]pyrimidine-carboxamide and (S)-2-chloro-N-(5-chloro-(2H-1,2,3-triazol-2-yl)-pyridin-3-yl)-8-(difluoromethyl)-8-methyl-7,8-dihydro-6H-pyrazolo[1,5-a]pyrrolo[2,3-e]pyrimidine-6-carboxamide

### Step 1: 1-(tert-butyl) 3-ethyl 3-methyl-4-oxopyrrolidine-1,3-dicarboxylate

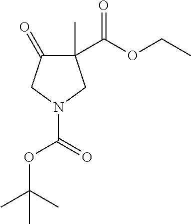 [SMILES: CCOC(=O)C1(C)CN(C(=O)OC(C)(C)C)CC1=O]

To a stirred solution of 1-(tert-butyl) 3-ethyl 4-oxopyrrolidine-1,3-dicarboxylate (400 g, 1.6 mol) in acetone (2000 mL) was added K₂CO₃ (430 g, 3.2 mol) and iodomethane (442 g, 3.2 mol). The resulting mixture was stirred at 50° C. for 16 h. The mixture was allowed to cool down to 25° C. The solid was filtered out. The filtrate was concentrated under vacuum. The residue was purified by column chromatography using 80% petroleum ether and 20% ethyl acetate as eluents to afford 1-(tert-butyl) 3-ethyl 3-methyl-4-oxopyrrolidine-1,3-dicarboxylate (320 g, 75.9% yield) as a yellow oil. ¹H NMR (400 MHz, DMSO-d₆) δ: 4.11-4.14 (m, 1H), 3.95-4.06 (m, 2H), 3.84-3.91 (m, 1H), 3.62-3.67 (m, 1H), 3.25-3.34 (d, J=3.6 Hz, 1H), 1.33 (s, 9H), 1.24 (s, 3H), 1.08-1.11 (m, 3H). LC-MS: m/z 272 [M+H]⁺.

### Step 2: 1-(tert-butyl) 3-ethyl 4-hydroxy-3-methylpyrrolidine-1,3-dicarboxylate

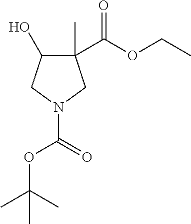 [SMILES: CCOC(=O)C1(C)CN(C(=O)OC(C)(C)C)CC1O]

To a stirred solution of 1-(tert-butyl) 3-ethyl 3-methyl-4-oxopyrrolidine-1,3-dicarboxylate (210 g, 774.0 mmol) in ethanol (1000 mL) was added NaBH₄ (30 g, 774.0 mmol) in portions at 0° C. The reaction was stirred at 0° C. for 1 h under nitrogen. The mixture was poured into water (1000 mL) and concentrated under vacuum. The mixture was extracted with ethyl acetate (3×1000 mL). The combined organic layers were washed with brine (500 mL), dried over anhydrous sodium sulfate and concentrated under vacuum to afford 1-(tert-butyl) 3-ethyl 4-hydroxy-3-methylpyrrolidine-1,3-dicarboxylate (98.7 g, 46.7% yield) as a yellow oil. LC-MS: m/z 274 [M+H]⁺.

### Step 3: 1-(tert-butyl) 3-ethyl 4-((tert-butyldimethylsilyl)oxy)-3-methyl pyrrolidine-1,3-dicarboxylate

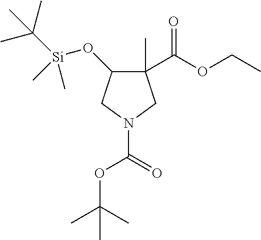 [SMILES: CCOC(=O)C1(C)CN(C(=O)OC(C)(C)C)CC1O[Si](C)(C)C(C)(C)C]

To a stirred solution of 1-(tert-butyl) 3-ethyl 4-hydroxy-3-methylpyrrolidine-1,3-dicarboxylate (98.7 g, 361.1 mmol) in N,N-dimethylformamide (500 mL) were added tert-butylchlorodimethylsilane (108.8 g, 722.2 mmol) and imidazole (98.3 g, 1.5 mol) at 25° C. The resulting mixture was stirred at 25° C. for 16 h. The mixture was poured into water (1000 mL) and extracted with ethyl acetate (3×1000 mL). The combined organic layers were washed with brine (1000 mL), dried over anhydrous sodium sulfate and concentrated under vacuum. The residue was purified by column chromatography on silica gel using 20% petroleum ether and 80% ethyl acetate as eluents to get the crude product. The crude product was purified by Prep-HPLC and the collected fractions were concentrated under vacuum to give 1-(tert-butyl) 3-ethyl 4-((tert-butyldimethylsilyl)oxy)-3-methylpyrrolidine-1,3-dicarboxylate (25.8 g, 16.4% yield) as a yellow oil. ¹H NMR (400 MHz, DMSO-d₆) δ: 4.47-4.50 (m, 1H), 4.07-4.17 (m, 2H), 3.69-3.74 (m, 1H), 3.46-3.58 (m, 1H), 3.10-3.33 (m, 2H), 1.43 (s, 9H), 1.23 (s, 6H), 0.85 (s, 9H), 0.07 (s, 6H). LC-MS: m/z 388 [M+H]⁺.

### Step 4: tert-butyl 4-((tert-butyldimethylsilyl)oxy)-3-(hydroxymethyl)-3-methylpyrrolidine-1-carboxylate

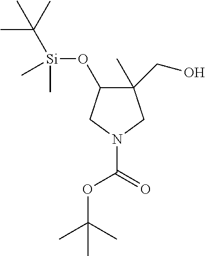 [SMILES: CC(C)(C)OC(=O)N1CC(O[Si](C)(C)C(C)(C)C)C(C)(CO)C1]

To a stirred solution of 1-(tert-butyl) 3-ethyl 4-((tert-butyldimethylsilyl)oxy)-3-methylpyrrolidine-1,3-dicarboxylate (25.8 g, 66.6 mmol) in tetrahydrofuran (200 mL) was added LiAlH₄ (2.6 g, 66.6 mmol) at 0° C. The resulting mixture was stirred for 0.5 h at 0° C. The mixture was added water (2.6 g) and 10% NaOH aqueous solution (2.6 g). The resulting mixture was filtered and concentrated under vacuum to afford tert-butyl 4-((tert-butyldimethylsilyl)oxy)-3-(hydroxymethyl)-3-methylpyrrolidine-1-carboxylate (21.2 g, 92.2% yield) as a yellow oil. LC-MS: m/z 346 [M+H]⁺.

### Step 5: tert-butyl 4-((tert-butyldimethylsilyl)oxy)-3-formyl-3-methylpyrrolidine-1-carboxylate

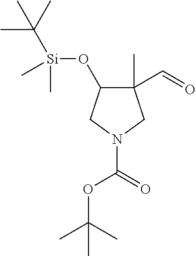 [SMILES: CC(C)(C)OC(=O)N1CC(O[Si](C)(C)C(C)(C)C)C(C)(C=O)C1]

To a stirred mixture of tert-butyl 4-((tert-butyldimethylsilyl)oxy)-3-(hydroxymethyl)-3-methylpyrrolidine-1-carboxylate (21.2 g, 61.3 mmol) in dichloromethane (500 mL) was added Dess-Martin Periodinane (52.1 g, 122.7 mmol) at 25° C. The reaction mixture was stirred at 25° C. for 1.5 h. The reaction mixture was quenched with water (800 mL). The resulting solution was extracted with ethyl acetate (3×800 mL). The combined organic layers were washed with brine (600 mL), and dried over anhydrous sodium sulfate and concentrated under vacuum. The residue was purified by column chromatography on silica gel using 50% petroleum ether and 50% ethyl acetate as eluents to afford tert-butyl 4-((tert-butyldimethylsilyl)oxy)-3-formyl-3-methylpyrrolidine-1-carboxylate (11.1 g, 48.4% yield) as a yellow oil. LC-MS: m/z 344 [M+H]⁺.

### Step 6: tert-butyl 4-((tert-butyldimethylsilyl)oxy)-3-(difluoromethyl)-3-methylpyrrolidine-1-carboxylate

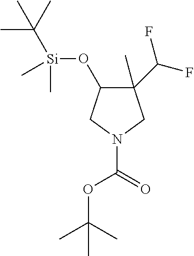 [SMILES: CC(C)(C)OC(=O)N1CC(O[Si](C)(C)C(C)(C)C)C(C)(C(F)F)C1]

To a stirred mixture of tert-butyl 4-((tert-butyldimethylsilyl)oxy)-3-formyl-3-methylpyrrolidine-1-carboxylate (11.1 g, 32.3 mmol) in dichloromethane (200 mL) was added DAST (15.6 g, 96.9 mmol) at 0° C. The reaction mixture was stirred at 0° C. for 2 h. The reaction mixture was warmed up to 25° C. and stirred for 16 h. The reaction mixture was quenched with water (500 mL). The resulting solution was extracted with ethyl acetate (3×500 mL). The combined organic layers were dried over anhydrous sodium sulfate and concentrated under vacuum. The residue was purified by column chromatography using 80% petroleum ether and 20% ethyl acetate as eluents to afford tert-butyl 4-((tert-butyldimethylsilyl)oxy)-3-(difluoromethyl)-3-methylpyrrolidine-1-carboxylate (5.5 g, 46.6% yield) as a yellow oil. LC-MS: m/z 366 [M+H]⁺.

### Step 7: tert-butyl 3-(difluoromethyl)-4-hydroxy-3-methylpyrrolidine-1-carboxylate

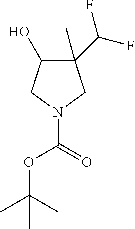 [SMILES: CC(C)(C)OC(=O)N1CC(O)C(C)(C(F)F)C1]

To a stirred mixture of tert-butyl 4-((tert-butyldimethylsilyl)oxy)-3-(difluoromethyl)-3-methylpyrrolidine-1-carboxylate (5.5 g, 15.0 mmol) in tetrahydrofran (100 mL) was added TBAF (62 mL, 1 M in tetrahydrofuran) at 25° C. The reaction mixture was stirred at 25° C. for 2 h. The reaction mixture was quenched with water (200 mL). The resulting solution was extracted with ethyl acetate (3×200 mL). The combined organic layers were dried over anhydrous sodium sulfate and concentrated under vacuum. The residue was purified by column chromatography using 80% petroleum ether and 20% ethyl acetate as eluents to afford tert-butyl 3-(difluoromethyl)-4-hydroxy-3-methylpyrrolidine-1-carboxylate (1.1 g, 28.3% yield) as a yellow oil. LC-MS: m/z 252 [M+H]⁺.

### Step 8. tert-butyl 3-(difluoromethyl)-3-methyl-4-oxopyrrolidine-1-carboxylate

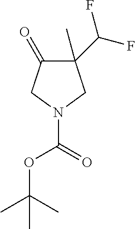 [SMILES: CC(C)(C)OC(=O)N1CC(=O)C(C)(C(F)F)C1]

To a stirred mixture of tert-butyl 3-(difluoromethyl)-4-hydroxy-3-methylpyrrolidine-1-carboxylate (1.1 g, 4.2 mmol) in dichloromethane (100 mL) was added PCC (4.6 g, 21.3 mmol) and SiO₂ (4.6 g) at 25° C. The reaction mixture was stirred at 50° C. for 16 h. The solid was filtered out. The filtrate was concentrated under vacuum. The residue was purified by column chromatography using 60% petroleum ether and 40% ethyl acetate as eluents to afford tert-butyl 3-(difluoromethyl)-3-methyl-4-oxopyrrolidine-1-carboxylate (500 mg, 47.1% yield) as a yellow oil. LC-MS: m/z 250 [M+H]⁺.

### Step 9. tert-butyl (Z)-4-(difluoromethyl)-2-((dimethylamino)methylene)-4-methyl-3-oxopyrrolidine-1-carboxylate

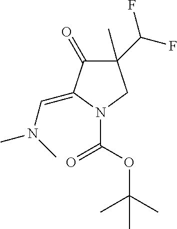 [SMILES: CN(C)/C=C1/C(=O)C(C)(C(F)F)CN1C(=O)OC(C)(C)C]

A mixture of tert-butyl 3-(difluoromethyl)-3-methyl-4-oxopyrrolidine-1-carboxylate (500 mg, 2.0 mmol) in DMF-DMA (25 mL) was stirred at 35° C. for 1 h. The reaction mixture was concentrated under vacuum to afford tert-butyl (Z)-4-(difluoromethyl)-2-((dimethylamino)methylene)-4-methyl-3-oxopyrrolidine-1-carboxylate (700 mg, crude) as a brown solid. LC-MS: m/z 305 [M+H]⁺.

### Step 10. tert-butyl 2-chloro-8-(difluoromethyl)-8-methyl-7,8-dihydro-6H-pyrazolo[1,5-a]pyrrolo[2,3-e]pyrimidine-6-carboxylate

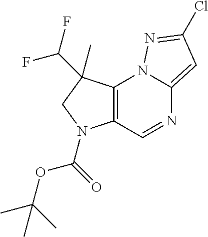 [SMILES: CC(C)(C)OC(=O)N1CC(C)(C(F)F)c2c1cnc1cc(Cl)nn21]

To a stirred solution of tert-butyl (Z)-4-(difluoromethyl)-2-((dimethylamino)methylene)-4-methyl-3-oxopyrrolidine-1-carboxylate (700 mg, 2.3 mmol) in toluene (20 mL) was added acetic acid (2 mL) and 3-chloro-1H-pyrazol-5-amine (324.4 mg, 2.7 mmol). The reaction mixture was stirred at 95° C. for 16 h. The mixture was concentrated under vacuum. The residue was purified by column chromatography using 80% petroleum ether and 20% ethyl acetate as eluents to afford tert-butyl 2-chloro-8-(difluoromethyl)-8-methyl-7,8-dihydro-6H-pyrazolo[1,5-a]pyrrolo[2,3-e]pyrimidine-6-carboxylate (220 mg, 26.1% yield) as a yellow oil. LC-MS: m/z 359 [M+H]⁺.

### Step 11. 2-chloro-8-(difluoromethyl)-8-methyl-7,8-dihydro-6H-pyrazolo[1,5-a]pyrrolo[2,3-e]pyrimidine

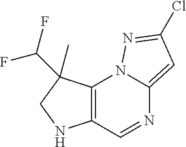 [SMILES: CC1(C(F)F)CNc2cnc3cc(Cl)nn3c21]

To a solution of tert-butyl 2-chloro-8-(difluoromethyl)-8-methyl-7,8-dihydro-6H-pyrazolo[1,5-a]pyrrolo[2,3-e]pyrimidine-6-carboxylate (220 mg, 613.2 umol) in dichloromethane (15 mL) were added trifluoroacetic acid (3 mL) at 25° C. The reaction was stirred at 25° C. for 1 h. The residue was quenched by saturated aqueous NaHCO₃ solution (40 mL). The resulting mixture was extracted with ethyl acetate (3×50 mL). The combined organic layers were concentrated under vacuum. The residue was purified by column chromatography using 30% petroleum ether and 70% ethyl acetate as eluents to afford 2-chloro-8-(difluoromethyl)-8-methyl-7,8-dihydro-6H-pyrazolo[1,5-a]pyrrolo[2,3-e]pyrimidine (120 mg, 72.6% yield) as a brown solid. LC-MS: m/z 259 [M+H]⁺.

### Step 12: 2-chloro-N-(5-chloro-6-(2H-1,2,3-triazol-2-yl)pyridin-3-yl)-8-(difluoromethyl)-8-methyl-7,8-dihydro-6H-pyrazolo[1,5-a]pyrrolo[2,3-e]pyrimidine-6-carboxamide

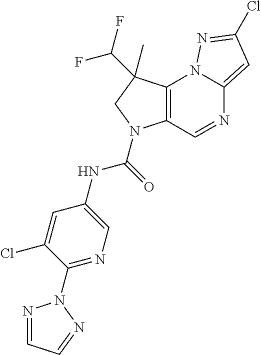 [SMILES: CC1(C(F)F)CN(C(=O)Nc2cnc(-n3nccn3)c(Cl)c2)c2cnc3cc(Cl)nn3c21]

To a stirred solution of 5-chloro-6-(2H-1,2,3-triazol-2-yl)pyridin-3-amine (Method A1 step 2; 137 mg, 698.6 μmol) in tetrahydrofuran (3 mL) were added triphosgene (83 mg, 279.4 μmol) and TEA (142 mg, 1.4 mmol). The resulting mixture was stirred at 25° C. for 0.5 h and then filtered. The filtrate was added to a solution of 2-chloro-8-(difluoromethyl)-8-methyl-7,8-dihydro-6H-pyrazolo[1,5-a]pyrrolo[2,3-e]pyrimidine (120 mg, 465.7 μmol) in tetrahydrofuran (1 mL). The mixture was stirred at 25° C. for 2 h. The mixture was concentrated under vacuum. The reaction mixture was quenched by the addition of water (50 mL). The resulting solution was extracted with ethyl acetate (3×50 mL). The combined organic layers were dried over anhydrous sodium sulfate and concentrated under vacuum. The residue was purified by column chromatography on silica gel using 10% methanol and 90% dichloromethane as eluents to afford crude product. The residue was purified by Prep-HPLC and the collected fractions were lyophilized to give 2-chloro-N-(5-chloro-6-(2H-1,2,3-triazol-2-yl)pyridin-3-yl)-8-(difluoromethyl)-8-methyl-7,8-dihydro-6H-pyrazolo[1,5-a]pyrrolo[2,3-e]pyrimidine-6-carboxamide (57 mg, 117.5 umol) as an off-white solid. LC-MS: m/z 480 [M+H]⁺.

### Step 13: Separation of enantiomers to obtain (R)-2-chloro-N-(5-chloro-6-(2H-1,2,3-triazol-2-yl)pyridin-3-yl)-8-(difluoromethyl)-8-methyl-7,8-dihydro-6H-pyrazolo[1,5-a]pyrrolo[2,3-e]pyrimidine-6-carboxamide and (S)-2-chloro-N-(5-chloro-6-(2H-1,2,3-triazol-2-yl)pyridin-3-yl)-8-(difluoromethyl)-8-methyl-7,8-dihydro-6H-pyrazolo[1,5-a]pyrrolo[2,3-e]pyrimidine-6-carboxamide

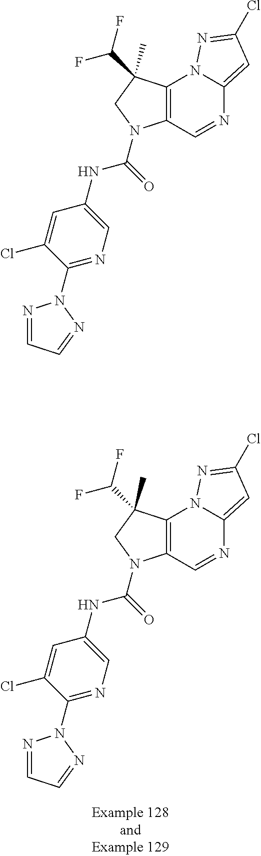 [SMILES: C[C@@]1(C(F)F)CN(C(=O)Nc2cnc(-n3nccn3)c(Cl)c2)c2cnc3cc(Cl)nn3c21 ; C[C@]1(C(F)F)CN(C(=O)Nc2cnc(-n3nccn3)c(Cl)c2)c2cnc3cc(Cl)nn3c21]

2-chloro-N-(5-chloro-6-(2H-1,2,3-triazol-2-yl)pyridin-3-yl)-8-(difluoromethyl)-8-methyl-7,8-dihydro-6H-pyrazolo[1,5-a]pyrrolo[2,3-e]pyrimidine-6-carboxamide (57 mg, 117.5 μmol) was submitted to chiral-HPLC: Column: CHIRALPAK IA, 2×25 cm, 5 um; Mobile Phase A: Hex(0.5% 2M NH₃-MeOH)-HPLC, Mobile Phase B:EtOH—HPLC; Flow rate: 20 mL/min; Gradient: 20 B to 20 B in 12 min; 254/220 nm; RT1:7.818; RT2:9.92; Injection Volume: 0.8 ml; Number Of Runs: 5. The first eluting isomer was concentrated and lyophilized to afford Example 129 (10 mg, 4.4% yield) as an off-white solid and the second eluting isomer was concentrated and lyophilized to afford Example 128 (10 mg, 4.4% yield) as an off-white solid. Examples 128 and 129 are enantiomers, but their absolute stereochemistry is not yet known.

Example 128: ¹H NMR (400 MHz, DMSO-d₆) δ: 9.64 (br, 1H), 9.31 (s, 1H), 8.77 (d, J=2.4 Hz, 1H), 8.53 (d, J=2.4 Hz, 1H), 8.17 (s, 2H), 7.03 (s, 1H), 6.80 (t, J=55.6 Hz, 1H), 4.70 (d, J=10.8 Hz, 1H), 4.20 (d, J=10.8 Hz, 1H), 1.80 (s, 3H). LC-MS: m/z 480 [M+H]⁺.

Example 129: ¹H NMR (400 MHz, DMSO-d₆) δ: 9.64 (br, 1H), 9.31 (s, 1H), 8.77 (d, J=2.4 Hz, 1H), 8.53 (d, J=2.4 Hz, 1H), 8.17 (s, 2H), 7.03 (s, 1H), 6.79 (t, J=55.2 Hz, 1H), 4.70 (d, J=10.8 Hz, 1H), 4.20 (d, J=10.8 Hz, 1H), 1.80 (s, 3H). LC-MS: m/z 480 [M+H]⁺.

### Method O4

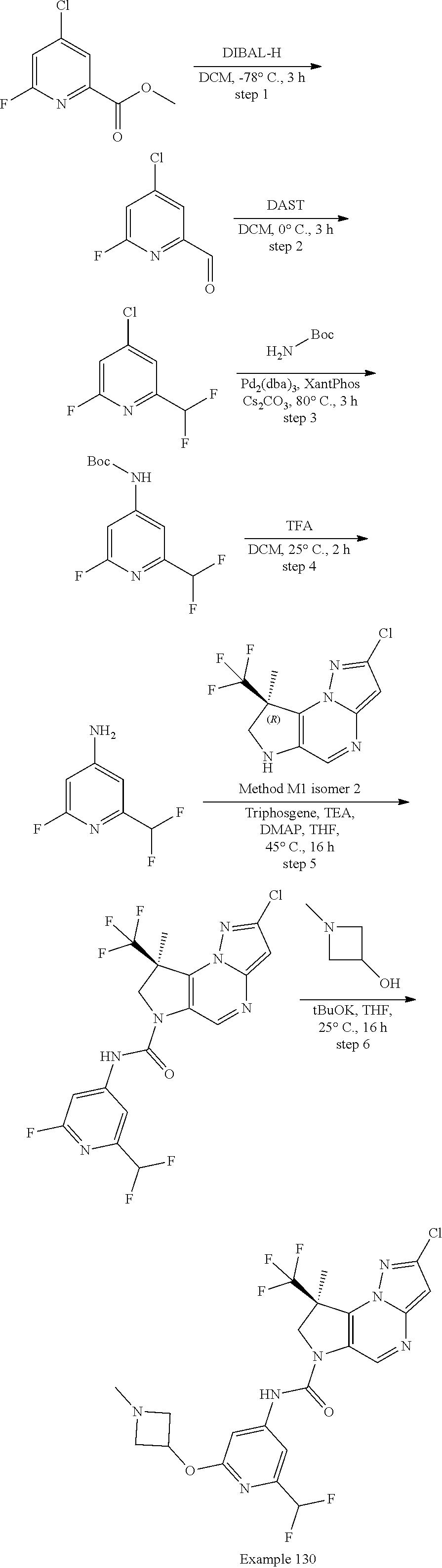

### Example 130: (R)-2-chloro-N-(2-(difluoromethyl)-6-((1-methylazetidin-3-yl)oxy)pyridin-4-yl)-8-methyl-8-(trifluoromethyl)-7,8-dihydro-6H-pyrazolo[1,5-a]pyrrolo[2,3-e]pyrimidine-6-carboxamide

### Step 1: 4-chloro-6-fluoropicolinaldehyde

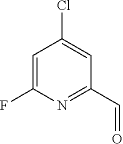

To a stirred solution of methyl 4-chloro-6-fluoropicolinate (4.0 g, 21.1 mmol) in dichloromethane (50 mL) was added diisobutylaluminum hydride (21 mL, 21.1 mmol, 1M in dichloromethane) at −78° C. The resulting mixture was stirred for 3 h at −78° C. The reaction mixture was quenched with saturated aqueous potassium sodium tartrate (100 mL) at −78° C. The mixture was allowed to warm up to 25° C. and filtered. The filtrate was washed with dichloromethane (3×100 mL). The combined organic layers were dried over anhydrous sodium sulfate and concentrated under vacuum. The residue was purified by column chromatography on silica gel using 85% petroleum ether and 15% ethyl acetate as eluents to afford 4-chloro-6-fluoropicolinaldehyde (1.5 g, 45% yield) as a white solid. ¹H NMR (400 MHz, Chloroform-d) δ: 9.91 (s, 1H), 7.84-7.86 (m, 1H), 7.22-7.26 (m, 1H). LC-MS: m/z 160 [M+H]⁺.

### Step 2: 4-chloro-2-(difluoromethyl)-6-fluoropyridine

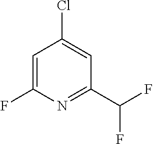 [SMILES: Fc1cc(Cl)cc(C(F)F)n1]

To a solution of 4-chloro-6-fluoropicolinaldehyde (1.5 g, 9.4 mmol) in dichloromethane (20 mL) were added DAST (3 g, 18.8 mmol) at −30° C. The resulting mixture was stirred at 0° C. for 3 h. The reaction mixture was quenched by the addition of water (150 mL). The resulting solution was extracted with dichloromethane (3×150 mL). The combined organic layers were dried over anhydrous sodium sulfate and concentrated under vacuum to give 4-chloro-2-(difluoromethyl)-6-fluoropyridine (1.0 g, 54% yield) as a colorless oil. ¹H NMR (400 MHz, Chloroform-d) δ: 7.55 (s, 1H), 7.10-7.12 ((m, 1H), 6.52 (t, J=56 Hz, 1H). LC-MS: m/z 182 [M+H]⁺.

### Step 3: tert-butyl (2-(difluoromethyl)-6-fluoropyridin-4-yl)carbamate

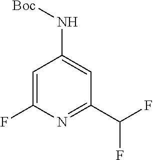 [SMILES: CC(C)(C)OC(=O)Nc1cc(F)nc(C(F)F)c1]

To a stirred solution of 4-chloro-2-(difluoromethyl)-6-fluoropyridine (1.0 g, 5.5 mmol) and tert-butyl carbamate (1.3 g, 11.1 mmol) in dioxane (20 mL) were added XantPhos (637 mg, 1.1 mmol), Pd₂(dba)₃ (570 mg, 550.8 μmol) and Cs₂CO₃ (3.6 g, 11.1 mmol) under nitrogen atmosphere. The resulting mixture was stirred at 80° C. for 3 h. The reaction mixture was cooled to 25° C. The solid was filtered out. The filtrate was concentrated under vacuum. The residue was purified by column chromatography on silica gel using 70% petroleum ether and 30% ethyl acetate as eluents to afford tert-butyl (2-(difluoromethyl)-6-fluoropyridin-4-yl)carbamate (800 mg, 55% yield) as a white solid. ¹H NMR (400 MHz, Chloroform-d) δ: 7.75 (br, 1H), 7.55 (s, 1H), 7.10-7.12 (m, 1H), 6.45 (t, J=56 Hz, 1H), 1.51 (s, 9H). LC-MS: m/z 263 [M+H]⁺.

### Step 4: 2-(difluoromethyl)-6-fluoropyridin-4-amine

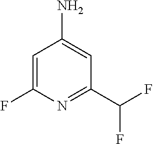 [SMILES: Nc1cc(F)nc(C(F)F)c1]

To a stirred solution of tert-butyl (2-(difluoromethyl)-6-fluoropyridin-4-yl) carbamate (800 mg, 3.1 mmol) in dichloromethane (12 mL) was added TFA (3 mL). The mixture was stirred at 25° C. for 2 h. The resulting mixture was concentrated under vacuum. The residue was added saturated aqueous NaHCO₃ solution (40 mL). The resulting solution was extracted with dichloromethane (3×40 mL). The combined organic layers were washed with brine (50 mL) and dried over anhydrous sodium sulfate and concentrated under vacuum. The residue was purified by column chromatography on silica gel using 40% petroleum ether and 60% ethyl acetate as eluents to afford 2-(difluoromethyl)-6-fluoropyridin-4-amine (400 mg, 79% yield) as a white solid. ¹H NMR (400 MHz, DMSO-d₆) δ: 7.90 (br, 2H), 6.73 (s, 1H), 6.45 (t, J=56 Hz, 1H), 6.14-6.16 (m, 1H). LC-MS: m/z 163 [M+H]⁺.

### Step 5: (R)-2-chloro-N-(2-(difluoromethyl)-6-fluoropyridin-4-yl)-8-methyl-8-(trifluoromethyl)-7,8-dihydro-6H-pyrazolo[1,5-a]pyrrolo[2,3-e]pyrimidine-6-carboxamide

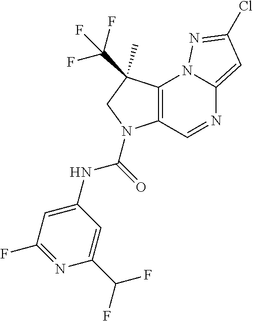 [SMILES: C[C@@]1(C(F)(F)F)CN(C(=O)Nc2cc(F)nc(C(F)F)c2)c2cnc3cc(Cl)nn3c21]

To a stirred solution of Method M1 isomer 2 (100 mg, 362.3 μmol) in tetrahydrofuran (4 mL) were added triphosgene (64 mg, 217.4 μmol) and TEA (49 mg, 489.4 μmol) at 0° C. The resulting mixture was stirred at 25° C. for 0.5 h and then filtered. The filtrate was added to a solution of tert-butyl 3-((5-amino-1-(difluoromethyl)-2-oxo-1,2-dihydropyridin-3-yl)oxy)azetidine-1-carboxylate (118 mg, 724.6 μmol) in tetrahydrofuran (2 mL). To this solution was then added N,N-dimethylpyridin-4-amine (88 mg, 724.6 μmol) and TEA (364 mg, 3.6 mmol). The mixture was stirred at 45° C. for 16 h. The reaction was cooled to 25° C. The mixture was poured into water (30 mL) and extracted with ethyl acetate (3×30 mL). The combined organic layers were washed with brine (50 mL), dried over anhydrous sodium sulfate and concentrated under vacuum. The residue was purified by Prep-TLC using 95% dichloromethane and 5% methanol as eluents to afford (R)-2-chloro-N-(2-(difluoromethyl)-6-fluoropyridin-4-yl)-8-methyl-8-(trifluoromethyl)-7,8-dihydro-6H-pyrazolo[1,5-a]pyrrolo[2,3-e]pyrimidine-6-carboxamide (80 mg, 47% yield) as a white solid. ¹H NMR (300 MHz, Methanol-d₄) δ: 9.39 (s, 1H), 7.43-7.52 (m, 1H), 6.79 (s, 1H), 6.52 (t, J=56 Hz, 1H), 5.35 (s, 1H), 4.43-4.45 (m, 2H), 2.03 (s, 3H). LC-MS: m/z 465 [M+H]⁺.

### Step 6: (R)-2-chloro-N-(2-(difluoromethyl)-6-((1-methylazetidin-3-yl)oxy)pyridin-4-yl)-8-methyl-8-(trifluoromethyl)-7,8-dihydro-6H-pyrazolo[1,5-a]pyrrolo[2,3-e]pyrimidine-6-carboxamide

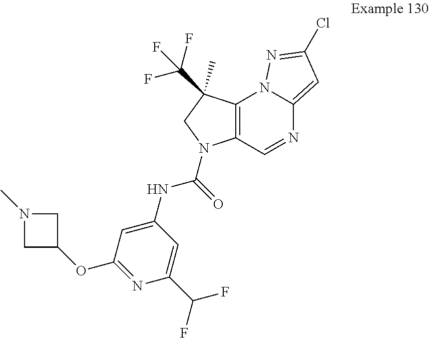 [SMILES: CN1CC(Oc2cc(NC(=O)N3C[C@@](C)(C(F)(F)F)c4c3cnc3cc(Cl)nn43)cc(C(F)F)n2)C1]

To a stirred solution of (R)-2-chloro-N-(2-(difluoromethyl)-6-fluoropyridin-4-yl)-8-methyl-8-(trifluoromethyl)-7,8-dihydro-6H-pyrazolo[1,5-a]pyrrolo[2,3-e]pyrimidine-6-carboxamide (30 mg, 172.0 μmol) in tetrahydrofuran (6 mL) was added 1-methylazetidin-3-ol (30 mg, 344.0 μmol) and potassium tert-butoxide (38 mg, 344.0 μmol). The mixture was stirred at 25° C. for 16 h. The resulting mixture was concentrated under vacuum. The residue was purified by Prep-HPLC purification and the collected fractions were lyophilized to give (R)-2-chloro-N-(2-(difluoromethyl)-6-((1-methylazetidin-3-yl)oxy)pyridin-4-yl)-8-methyl-8-(trifluoromethyl)-7,8-dihydro-6H-pyrazolo[1,5-a]pyrrolo[2,3-e]pyrimidine-6-carboxamide (2 mg, 2% yield) as a white solid. The enantiomer of Example 130 can be prepared analogously using Method M1 isomer 1.

Example 130: ¹H NMR (300 MHz, Methanol-d₄) δ: 9.35 (s, 1H), 7.52 (d, J=1.2 Hz, 1H), 7.43 (s, 1H), 6.79 (s, 1H), 6.53 (t, J=56 Hz, 1H), 5.38-5.42 (m, 1H), 4.78 (d, J=11.4 Hz, 1H), 4.43-4.45 (m, 2H), 4.17 (d, J=11.6 Hz, 1H), 4.03-4.05 (m, 2H), 2.86 (s, 3H), 2.03 (s, 3H). LC-MS: m/z 532 [M+H]⁺.

### Method P4

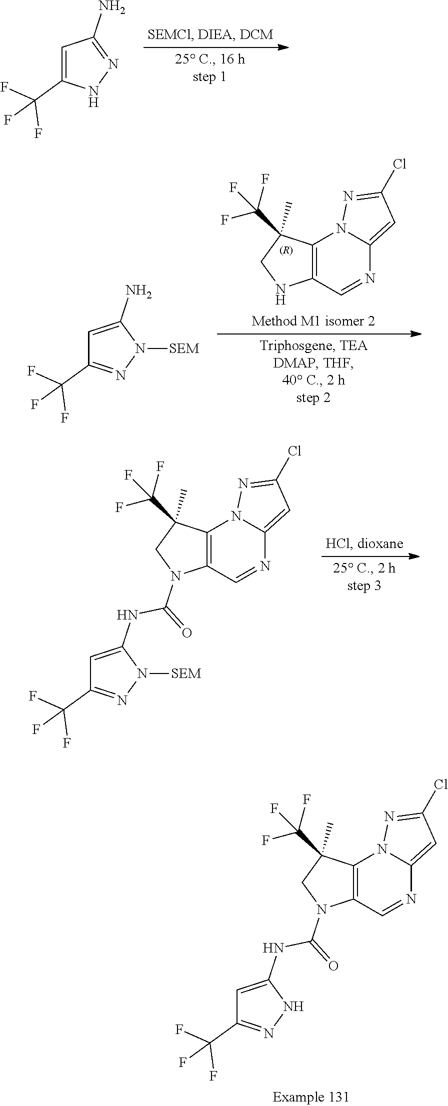

### Example 131: (R)-2-chloro-8-methyl-8-(trifluoromethyl)-N-(3-(trifluoromethyl)-1H-pyrazol-5-yl)-7,8-dihydro-6H-pyrazolo[1,5-a]pyrrolo[2,3-e]pyrimidine-6-carboxamide

### Step 1: 3-(trifluoromethyl)-1-((2-(trimethylsilyl)ethoxy)methyl)-1H-pyrazol-5-amine

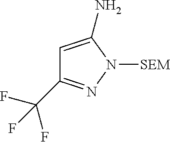 [SMILES: C[Si](C)(C)CCOCn1nc(C(F)(F)F)cc1N]

To a stirred mixture of 5-(trifluoromethyl)-1H-pyrazol-3-amine (1.0 g, 6.6 mmol) and DIEA (1.7 g, 13.2 mmol) in dichloromethane (20 mL) were added (2-(chloromethoxy)ethyl)trimethylsilane (1.1 g, 6.6 mmol) at 0° C. The reaction mixture was stirred at 25° C. for 16 h. The mixture was concentrated under vacuum. The residue was purified by column chromatography on silica gel using 88% petroleum ether and 12% ethyl acetate as eluents to afford 3-(trifluoromethyl)-1-((2-(trimethylsilyl)ethoxy)methyl)-1H-pyrazol-5-amine (870 mg, 37% yield) as a yellow oil. ¹H NMR (400 MHz, DMSO-d₆) δ: 6.05 (s, 1H), 5.23 (s, 2H), 5.12 (s, 2H), 3.49-3.54 (m, 2H), 0.76-0.83 (m, 2H), 0.03 (s, 9H). LC-MS: m/z 282 [M+H]⁺.

### Step 2: (R)-2-chloro-8-methyl-8-(trifluoromethyl)-N-(3-(trifluoromethyl)-1-((2-(trimethylsilyl)ethoxy)methyl)-1H-pyrazol-5-yl)-7,8-dihydro-6H-pyrazolo[1,5-a]pyrrolo[2,3-e]pyrimidine-6-carboxamide

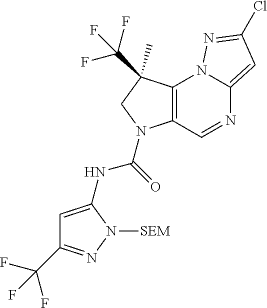 [SMILES: C[C@@]1(C(F)(F)F)CN(C(=O)Nc2cc(C(F)(F)F)nn2COCC[Si](C)(C)C)c2cnc3cc(Cl)nn3c21]

To a stirred solution of 3-(trifluoromethyl)-1-((2-(trimethylsilyl)ethoxy)methyl)-1H-pyrazol-5-amine (203 mg, 722.9 μmol) in tetrahydrofuran (2 mL) were added triphosgene (64 mg, 217 μmol) and TEA (55 mg, 542.2 μmol). The resulting mixture was stirred at 25° C. for 1 h and then filtered. The filtrate was added to a solution of Method M1 isomer 2 (100 mg, 361.5 μmol) in tetrahydrofuran (2 mL). To this solution were added N,N-dimethylpyridin-4-amine (66 mg, 542.2 μmol) and TEA (366 mg, 3.6 mmol). The mixture was stirred at 40° C. for 2 h. The mixture was concentrated under vacuum. The residue was purified by Prep-HPLC and the collected fractions were lyophilized to afford (R)-2-chloro-8-methyl-8-(trifluoromethyl)-N-(3-(trifluoromethyl)-1-((2-(trimethylsilyl)ethoxy)methyl)-1H-pyrazol-5-yl)-7,8-dihydro-6H-pyrazolo[1,5-a]pyrrolo[2,3-e]pyrimidine-6-carboxamide (200 mg, 89% yield) as a yellow solid. ¹H NMR (400 MHz, DMSO-d₆) δ: 10.22 (s, 1H), 9.31 (s, 1H), 7.08 (s, 1H), 7.05 (s, 1H), 5.48 (s, 2H), 4.86 (d, J=12.0 Hz, 1H), 4.19 (d, J=12.0 Hz, 1H), 3.59 (t, J=8.0 Hz, 2H), 1.94 (s, 3H), 0.85 (t, J=8.0 Hz, 2H), 0.03 (s, 9H). LC-MS: m/z 584 [M+H]⁺.

### Step 3: (R)-2-chloro-8-methyl-8-(trifluoromethyl)-N-(3-(trifluoromethyl)-1H-pyrazol-5-yl)-7,8-dihydro-6H-pyrazolo[1,5-a]pyrrolo[2,3-e]pyrimidine-6-carboxamide

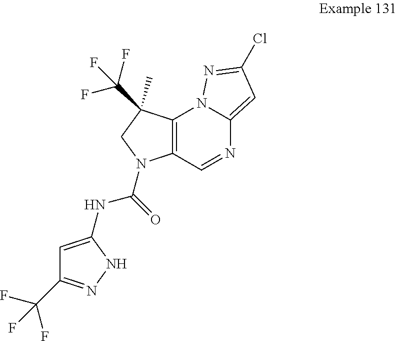 [SMILES: C[C@@]1(C(F)(F)F)CN(C(=O)Nc2cc(C(F)(F)F)n[nH]2)c2cnc3cc(Cl)nn3c21]

To a stirred mixture of (R)-2-chloro-8-methyl-8-(trifluoromethyl)-N-(3-(trifluoromethyl)-1-((2-(trimethylsilyl)ethoxy)methyl)-1H-pyrazol-5-yl)-7,8-dihydro-6H-pyrazolo[1,5-a]pyrrolo[2,3-e]pyrimidine-6-carboxamide (100 mg, 171.2 μmol) in dioxane (1 mL) were added HCl (10 mL, 40.0 mmol, 4M in dioxane). The resulting mixture was stirred at 25° C. for 2 h. The reaction mixture was concentrated under reduced pressure. The residue was purified by Prep-HPLC and the collected fractions were lyophilized to afford (R)-2-chloro-8-methyl-8-(trifluoromethyl)-N-(3-(trifluoromethyl)-1H-pyrazol-5-yl)-7,8-dihydro-6H-pyrazolo[1,5-a]pyrrolo[2,3-e]pyrimidine-6-carboxamide (16.6 mg, 20% yield) as a white solid. The enantiomer of Example 131 can be prepared analogously using Method M1 isomer 1.

Example 131: ¹H NMR (400 MHz, Chloroform-d) δ: 9.43 (s, 1H), 7.40 (s, 1H), 6.80 (s, 1H), 6.36 (s, 1H), 4.54 (d, J=10.4 Hz, 1H), 4.02 (d, J=10.4 Hz, 1H), 2.07 (s, 3H). LC-MS: m/z 454 [M+H]⁺.

### Method Q4

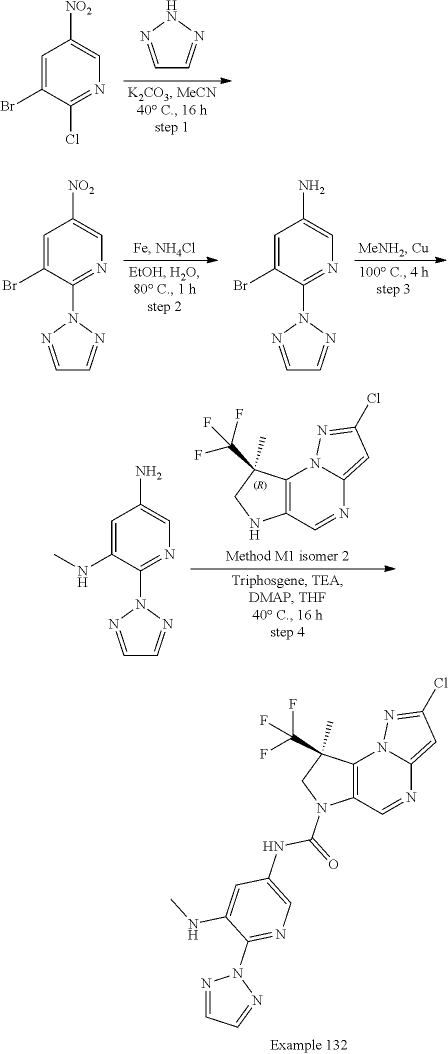

### Example 132: (R)-2-chloro-8-methyl-N-(5-(methylamino)-6-(2H-1,2,3-triazol-2-yl)pyridin-3-yl)-8-(trifluoromethyl)-7,8-dihydro-6H-pyrazolo[1,5-a]pyrrolo[2,3-e]pyrimidine-6-carboxamide

### Step 1: 3-bromo-5-nitro-2-(2H-1,2,3-triazol-2-yl)pyridine

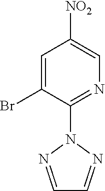 [SMILES: O=[N+]([O-])c1cnc(-n2nccn2)c(Br)c1]

To a stirred solution of 3-bromo-2-chloro-5-nitropyridine (10.0 g, 42.4 mmol) in acetonitrile (200 mL) were added 2H-1,2,3-triazole (3.2 g, 46.6 mmol) and K₂CO₃ (11.7 g, 84.7 mmol). The resulting mixture was stirred for 16 h at 40° C. The mixture was allowed to cool down to 25° C. The reaction mixture was filtered and the collected solid was washed with ethyl acetate (3×200 mL). The combined organic layers were concentrated under vacuum. The residue was purified by column chromatography on silica gel using 75% petroleum ether and 25% ethyl acetate as eluents to afford 3-bromo-5-nitro-2-(2H-1,2,3-triazol-2-yl)pyridine (2.5 g, 22% yield) as a yellow solid. ¹H NMR (300 MHz, DMSO-d₆) δ: 9.42 (d, J=2.4 Hz, 1H), 9.22 (d, J=2.4 Hz, 1H), 8.31 (s, 2H). LC-MS: m/z 270 [M+H]⁺.

### Step 2: 5-bromo-6-(2H-1,2,3-triazol-2-yl)pyridin-3-amine

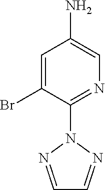 [SMILES: Nc1cnc(-n2nccn2)c(Br)c1]

To a solution of 3-bromo-5-nitro-2-(2H-1,2,3-triazol-2-yl)pyridine (1.0 g, 3.7 mmol) in ethanol (45 mL) and water (15 mL) were added Fe (1.0 g, 18.6 mmol) and NH₄Cl (0.8 g, 14.8 mmol). The resulting mixture was stirred at 80° C. for 1 h. The mixture was allowed to cool down to 25° C. The reaction mixture was filtered and the solid was washed with ethyl acetate (3×50 mL). The combined organic layers were concentrated under vacuum. The residue was purified by column chromatography on silica gel using 50% petroleum ether and 50% ethyl acetate as eluents to afford 5-bromo-6-(2H-1,2,3-triazol-2-yl)pyridin-3-amine (0.8 g, 89% yield) as a yellow oil. ¹H NMR (400 MHz, DMSO-d₆) δ: 8.01 (br, 2H) 7.82 (d, J=4 Hz, 1H), 7.34 (d, J=4 Hz, 1H), 6.14 (s, 2H); LC-MS: m/z 240 [M+H]⁺

### Step 3: N³-methyl-2-(2H-1,2,3-triazol-2-yl)pyridine-3,5-diamine

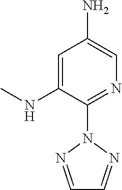

To a stirred solution of 5-bromo-6-(2H-1,2,3-triazol-2-yl)pyridin-3-amine (500 mg, 3.3 mmol) in methylamine (4 mL, 40% in water) was added copper (8 mg, 0.1 mmol). The reaction mixture was stirred at 100° C. for 4 h. The mixture was allowed to cool down to 25° C. The reaction mixture was quenched with water (20 mL). The resulting solution was extracted with ethyl acetate (3×50 mL). The combined organic layers were dried over anhydrous sodium sulfate and concentrated under vacuum. The residue was purified by column chromatography on silica gel using 10% methanol and 90% dichloromethane as eluents to afford N³-methyl-2-(2H-1,2,3-triazol-2-yl)pyridine-3,5-diamine (280 mg, 71% yield) as a yellow solid. ¹H NMR (400 MHz, DMSO-&) S: 7.98 (s, 2H), 7.11 (d, J=4 Hz, 1H), 6.32 (s, J=4 Hz, 1H), 5.57-5.59 (m, 1H), 5.49 (br, 2H), 2.67 (d, J=4 Hz, 3H). LC-MS: m/z 191 [M+H]⁺.

### Step 3: (R)-2-chloro-8-methyl-N-(5-(methylamino)-6-(2H-1,2,3-triazol-2-yl)pyridin-3-yl)-8-(trifluoromethyl)-7,8-dihydro-6H-pyrazolo[1,5-a]pyrrolo[2,3-e]pyrimidine-6-carboxamide

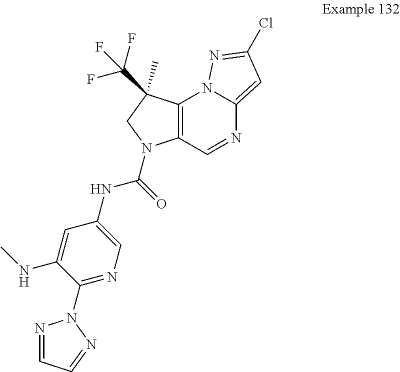 [SMILES: CNc1cc(NC(=O)N2C[C@@](C)(C(F)(F)F)c3c2cnc2cc(Cl)nn32)cnc1-n1nccn1]

To a stirred solution of N³-methyl-2-(2H-1,2,3-triazol-2-yl)pyridine-3,5-diamine (42 mg, 217 μmol) in tetrahydrofuran (8 mL) were added triphosgene (26 mg, 87 μmol) and TEA (22 mg, 217.4 μmol) at 25° C. The resulting mixture was stirred at 25° C. for 0.5 h and then filtered. The resulting filtrate was added to a solution of Method M1 isomer 2 (40 mg, 144.9 μmol) in tetrahydrofuran (1 mL). To this solution was then added TEA (146 mg, 1.4 mmol) and N,N-Dimethylpyridin-4-amine (2 mg, 14.5 μmol). The reaction mixture was stirred at 25° C. for 1 h. The reaction mixture was quenched with water (20 mL). The resulting solution was extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate as eluents to afford crude product. The crude product was submitted to Prep-HPLC purification and the collected fractions were lyophilized to give (R)-2-chloro-8-methyl-N-(5-(methylamino)-6-(2H-1,2,3-triazol-2-yl)pyridin-3-yl)-8-(trifluoromethyl)-7,8-dihydro-6H-pyrazolo[1,5-a]pyrrolo[2,3-e]pyrimidine-6-carboxamide (38.6 mg, 53% yield) as a white solid. The enantiomer of Example 132 can be prepared analogously using Method M1 isomer 1.

Example 132: ¹H NMR (400 MHz, DMSO-d₆) δ: 9.36 (s, 1H), 9.24 (s, 1H), 8.12 (s, 2H), 8.05 (d, J=2 Hz, 1H), 7.53 (d, J=2 Hz, 1H), 7.06 (s, 1H), 6.24-6.27 (m, 1H), 4.87 (d, J=12 Hz, 1H), 4.29 (d, J=12 Hz, 1H), 2.81 (d, J=4 Hz, 3H), 1.98 (s, 3H). LC-MS: m/z 493 [M+H]⁺.

### Method R4

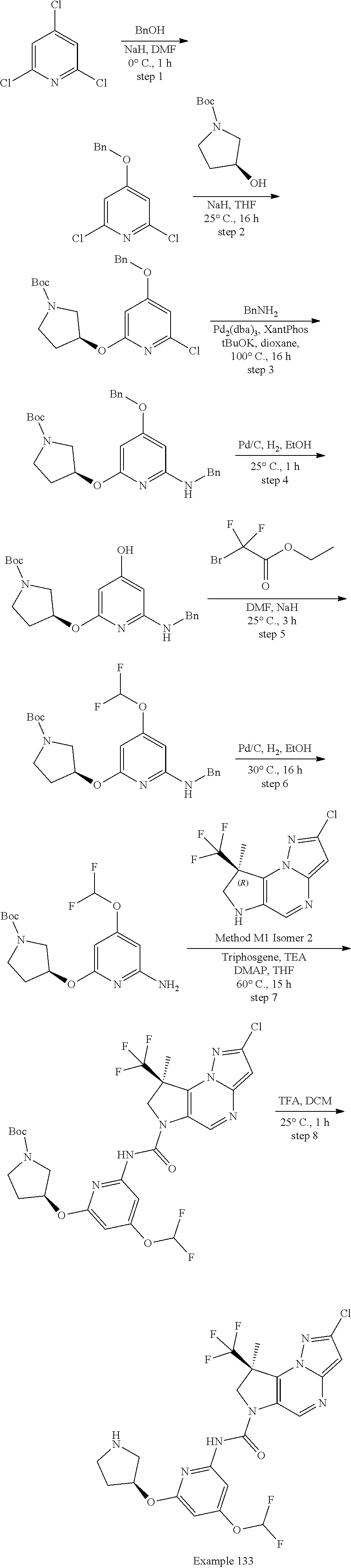

### Example 133: (R)-2-chloro-N-(4-(difluoromethoxy)-6-(((S)-pyrrolidin-3-yl)oxy)pyridin-2-yl)-8-methyl-8-(trifluoromethyl)-7,8-dihydro-6H-pyrazolo[1,5-a]pyrrolo[2,3-e]pyrimidine-6-carboxamide

### Step 1: 4-(benzyloxy)-2,6-dichloropyridine

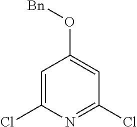 [SMILES: Clc1cc(OCc2ccccc2)cc(Cl)n1]

To a stirred solution of phenylmethanol (5.9 g, 54.8 mmol) in DMF (50 mL) was added NaH (2.4 g, 54.8 mmol, 60% in mineral oil) in batches at 0° C. The reaction mixture was stirred at 0° C. for 10 min. Then 2,4,6-trichloropyridine (10 g, 54.8 mmol) was added at 0° C. and the reaction mixture was stirred at 0° C. for 1 h. The reaction mixture was quenched with water (50 mL). The resulting solution was extracted with ethyl acetate (3×50 mL). The combined organic layers were dried over anhydrous sodium sulfate and concentrated under vacuum. The residue was purified by column chromatography on silica gel using 80% petroleum ether and 20% ethyl acetate as eluents to afford 4-(benzyloxy)-2,6-dichloropyridine (7 g, 45% yield) as a white solid. LC-MS: m/z 254 [M+H]⁺.

### Step 2: tert-butyl (S)-3-((4-(benzyloxy)-6-chloropyridin-2-yl)oxy)pyrrolidine-1-carboxylate

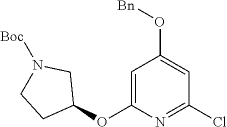 [SMILES: CC(C)(C)OC(=O)N1CC[C@H](Oc2cc(OCc3ccccc3)cc(Cl)n2)C1]

To a stirred solution of tert-butyl (S)-3-hydroxypyrrolidine-1-carboxylate (5.2 g, 27.5 mmol) in tetrahydrofuran (50 mL) was added NaH (1.1 g, 26.4 mmol, 60% in mineral oil) in batches at 0° C. The reaction mixture was stirred at 0° C. for 10 min. Then 4-(benzyloxy)-2,6-dichloropyridine (7 g, 27.5 mmol) was added at 0° C. and the reaction mixture was stirred at 25° C. for 16 h. The reaction mixture was quenched with water (100 mL). The resulting solution was extracted with ethyl acetate (3×100 mL). The combined organic layers were dried over anhydrous sodium sulfate and concentrated under vacuum. The residue was purified by column chromatography on silica gel using 85% petroleum ether and 15% ethyl acetate as eluents to afford tert-butyl (S)-3-((4-(benzyloxy)-6-chloropyridin-2-yl)oxy)pyrrolidine-1-carboxylate (3.7 g, 33% yield) as a colorless oil. LC-MS: m/z 405 [M+H]⁺.

### Step 3: tert-butyl (S)-3-((6-(benzylamino)-4-(benzyloxy)pyridin-2-yl)oxy)pyrrolidine-1-carboxylate

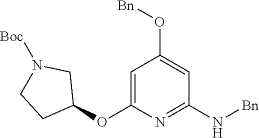 [SMILES: CC(C)(C)OC(=O)N1CC[C@H](Oc2cc(OCc3ccccc3)cc(NCc3ccccc3)n2)C1]

To a mixture of tert-butyl (3S)-3-[(4-benzyloxy-6-chloro-2-pyridyl)oxy]pyrrolidine-1-carboxylate (2.0 g, 4.9 mmol) in dioxane (160 mL) was added benzylamine (582 mg, 5.4 mmol), Pd₂(dba)₃ (1.5 g, 1.5 mmol), Xantphos (857 mg, 1.5 mmol) and t-BuOK (1.7 g, 14.8 mmol) under nitrogen atmosphere. The resulting mixture was stirred at 100° C. for 16 h. The reaction mixture was cooled to 25° C. and concentrated under vacuum. The resulting mixture was diluted with water (100 mL) and extracted with ethyl acetate (3×100 mL). The combined organic layers were dried over anhydrous sodium sulfate and concentrated under vacuum. The residue was purified by column chromatography on silica gel using 75% petroleum ether and 25% ethyl acetate to afford tert-butyl (S)-3-((6-(benzylamino)-4-(benzyloxy)pyridin-2-yl)oxy)pyrrolidine-1-carboxylate (1.0 g, 28% yield) as a yellow oil. LC-MS: m/z 476 [M+H]⁺.

### Step 4: tert-butyl (S)-3-((6-(benzylamino)-4-hydroxypyridin-2-yl)oxy)pyrrolidine-1-carboxylate

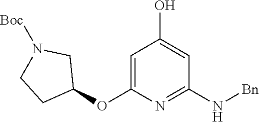 [SMILES: CC(C)(C)OC(=O)N1CC[C@H](Oc2cc(O)cc(NCc3ccccc3)n2)C1]

To a stirred solution of tert-butyl (3S)-3-[[6-(benzylamino)-4-benzyloxy-2-pyridyl]oxy]pyrrolidine-1-carboxylate (3.5 g, 3.8 mmol) in ethanol (30 mL) was added Pd/C (924 mg, 10%). The reaction mixture was stirred at 25° C. for 1 h under hydrogen. The solid was filtered out. The filtrate was concentrated under vacuum. The residue was purified by column chromatography on silica gel using 75% petroleum ether and 25% ethyl acetate as eluents to afford tert-butyl (S)-3-((6-(benzylamino)-4-hydroxypyridin-2-yl)oxy)pyrrolidine-1-carboxylate (450 mg, 31% yield) as a yellow solid. LC-MS: m/z 386 [M+H]⁺.

### Step 5: tert-butyl (S)-3-((6-(benzylamino)-4-(difluoromethoxy)pyridin-2-yl)oxy)pyrrolidine-1-carboxylate

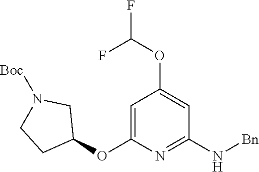 [SMILES: CC(C)(C)OC(=O)N1CC[C@H](Oc2cc(OC(F)F)cc(NCc3ccccc3)n2)C1]

To a stirred solution of tert-butyl (S)-3-((6-(benzylamino)-4-hydroxypyridin-2-yl)oxy)pyrrolidine-1-carboxylate (450 mg, 1.2 mmol) in DMF (5 mL) was added NaH (93 mg, 2.3 mmol, 60% in mineral oil) in batches at 0° C. The reaction mixture was stirred at 0° C. for 10 min. Then ethyl 2-bromo-2,2-difluoro-acetate (355 mg, 1.7 mmol) was added at 0° C. and the reaction mixture was stirred at 25° C. for 3 h. The reaction mixture was quenched with water (20 mL). The resulting solution was extracted with ethyl acetate (3×30 mL). The combined organic layers were dried over anhydrous sodium sulfate and concentrated under vacuum. The residue was purified by column chromatography on silica gel using 75% petroleum ether and 25% ethyl acetate as eluents to afford tert-butyl (S)-3-((6-(benzylamino)-4-(difluoromethoxy)pyridin-2-yl)oxy)pyrrolidine-1-carboxylate (68 mg, 13% yield) as a colorless oil. LC-MS: m/z 436 [M+H]⁺.

### Step 6: tert-butyl (S)-3-((6-amino-4-(difluoromethoxy)pyridin-2-yl)oxy)pyrrolidine-1-carboxylate

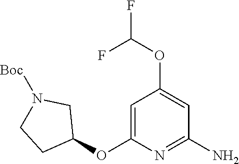 [SMILES: CC(C)(C)OC(=O)N1CC[C@H](Oc2cc(OC(F)F)cc(N)n2)C1]

To a stirred solution of tert-butyl (S)-3-((6-(benzylamino)-4-(difluoromethoxy)pyridin-2-yl)oxy)pyrrolidine-1-carboxylate (68 mg, 148.4 μmol) in ethanol (5 mL) was added Pd/C (50 mg, 10%). The reaction mixture was stirred at 30° C. for 16 h under hydrogen. The solid was filtered out. The filtrate was concentrated under vacuum. The residue was purified by column chromatography on silica gel using 70% petroleum ether and 30% ethyl acetate as eluents to afford tert-butyl (S)-3-((6-amino-4-(difluoromethoxy)pyridin-2-yl)oxy)pyrrolidine-1-carboxylate (30 mg, 44% yield) as a colorless oil. LC-MS: m/z 346 [M+H]⁺.

### Step 7: tert-butyl (S)-3-((6-((R)-2-chloro-8-methyl-8-(trifluoromethyl)-7,8-dihydro-6H-pyrazolo[1,5-a]pyrrolo[2,3-e]pyrimidine-6-carboxamido)-4-(difluoromethoxy)pyridin-2-yl)oxy)pyrrolidine-1-carboxylate

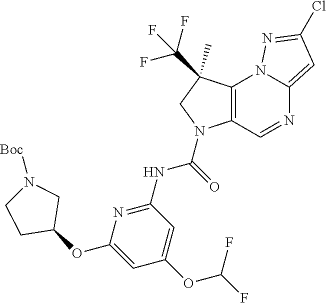 [SMILES: CC(C)(C)OC(=O)N1CC[C@H](Oc2cc(OC(F)F)cc(NC(=O)N3C[C@@](C)(C(F)(F)F)c4c3cnc3cc(Cl)nn43)n2)C1]

To a stirred solution of Method M1 isomer 2 (22 mg, 79.5 μmol) in tetrahydrofran (1 mL) were added triphosgene (14 mg, 47.4 μmol) and TEA (16 mg, 158.1 μmol). The resulting mixture was stirred at 25° C. for 1 h and then filtered. The filtrate was added to a solution of tert-butyl (S)-3-((6-amino-4-(difluoromethoxy)pyridin-2-yl)oxy)pyrrolidine-1-carboxylate (30 mg, 87.5 μmol) in tetrahydrofuran (1 mL). To this solution were added N,N-dimethylpyridin-4-amine (21 mg, 172.9 μmol) and TEA (26 mg, 256.9 μmol). The mixture was stirred at 60° C. for 15 h. The mixture was concentrated under vacuum. The residue was diluted with water (10 mL). The resulting solution was extracted with ethyl acetate (3×10 mL). The organic layers were combined, dried over anhydrous sodium sulfate and concentrated under vacuum. The residue was purified by column chromatography on silica gel using 70% petroleum ether and 30% ethyl acetate as eluents to afford tert-butyl (S)-3-((6-((R)-2-chloro-8-methyl-8-(trifluoromethyl)-7,8-dihydro-6H-pyrazolo[1,5-a]pyrrolo[2,3-e]pyrimidine-6-carboxamido)-4-(difluoromethoxy)pyridin-2-yl)oxy)pyrrolidine-1-carboxylate (55 mg, 45% yield) as a yellow solid. LC-MS: m/z 648 [M+H]⁺.

### Step 8: (R)-2-chloro-N-(4-(difluoromethoxy)-6-(((S)-pyrrolidin-3-yl)oxy)pyridin-2-yl)-8-methyl-8-(trifluoromethyl)-7,8-dihydro-6H-pyrazolo[1,5-a]pyrrolo[2,3-e]pyrimidine-6-carboxamide

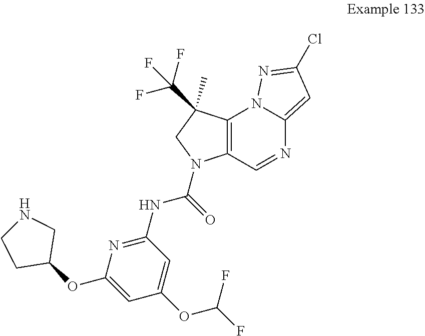 [SMILES: C[C@@]1(C(F)(F)F)CN(C(=O)Nc2cc(OC(F)F)cc(O[C@H]3CCNC3)n2)c2cnc3cc(Cl)nn3c21]

To a stirred solution of tert-butyl (S)-3-((6-((R)-2-chloro-8-methyl-8-(trifluoromethyl)-7,8-dihydro-6H-pyrazolo[1,5-a]pyrrolo[2,3-e]pyrimidine-6-carboxamido)-4-(difluoromethoxy)pyridin-2-yl)oxy)pyrrolidine-1-carboxylate (53 mg, 34.4 μmol) in dichloromethane (3 mL) was added TFA (1 mL). The reaction mixture was stirred at 25° C. for 1 h. The mixture was concentrated under vacuum. The residue was purified by Prep-HPLC and the collected fractions were lyophilized to afford (R)-2-chloro-N-(4-(difluoromethoxy)-6-(((S)-pyrrolidin-3-yl)oxy)pyridin-2-yl)-8-methyl-8-(trifluoromethyl)-7,8-dihydro-6H-pyrazolo[1,5-a]pyrrolo[2,3-e]pyrimidine-6-carboxamide (11 mg, 58% yield) as an off-white solid. The enantiomer of Example 133 can be prepared analogously using Method M1 isomer 1.

Example 133: ¹H NMR (400 MHz, DMSO-d₆) δ: 9.30 (s, 1H), 7.43 (t, J=73.2 Hz, 1H), 7.29 (s, 1H), 7.06 (s, 1H), 6.28 (s, 1H), 5.43 (s, 1H), 4.97 (d, J=11.6 Hz, 1H), 4.25 (d, J=12 Hz, 1H), 3.20-3.28 (m, 2H), 2.83-3.02 (m, 3H), 2.07-2.11 (m, 1H), 1.94 (s, 3H), 1.80-1.89 (m, 1H). LC-MS: m/z 548 [M+H]⁺.

### Method S4

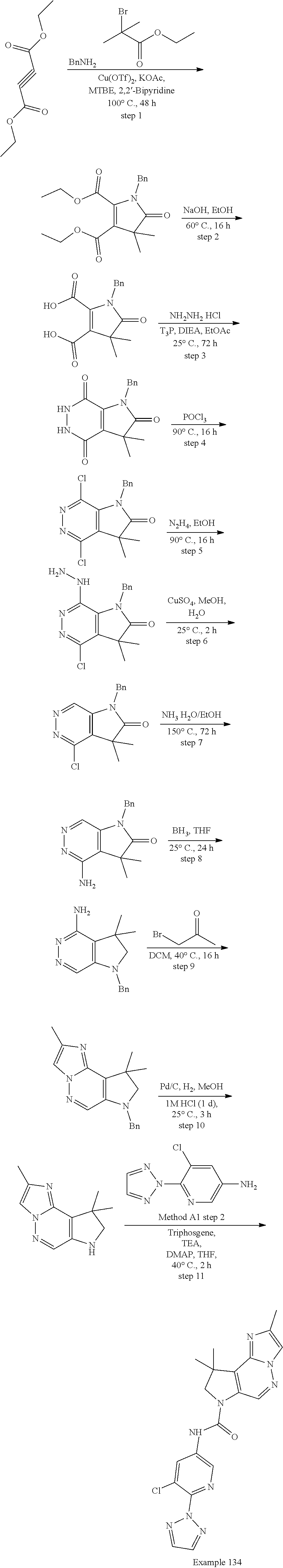

### Examples 134: N-(5-chloro-6-(2H-1,2,3-triazol-2-yl)pyridin-3-yl)-2,9,9-trimethyl-8,9-dihydro-7H-imidazo[1,2-b]pyrrolo[3,2-d]pyridazine-7-carboxamide

### Step 1: diethyl 1-benzyl-4,4-dimethyl-5-oxo-4,5-dihydro-1H-pyrrole-2,3-dicarboxylate

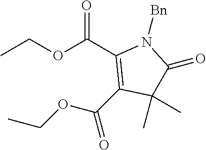 [SMILES: CCOC(=O)C1=C(C(=O)OCC)C(C)(C)C(=O)N1Cc1ccccc1]

To a stirred solution of phenylmethanamine (16.5 g, 154.3 mmol) in tert-Butyl methyl ether (300 mL) were added diethyl but-2-ynedioate (26.2 g, 154.3 mnmol) and ethyl 2-bromo-2-methylpropanoate (60.2 g, 308.5 mnmol) dropwised at 0° C. under nitrogen atmosphere. Then Cu(OTf)₂ (5.6 g, 15.4 mmol), 2,2′-Bipyridine (2.4 g, 15.4 mmol) and KOAc (15.1 g, 154.3 mmol) were added into the solution at 25° C. under nitrogen atmosphere. The reaction mixture was stirred at 100° C. for 48 h. The mixture was cooled to 25° C. The solvent was removed under vacuum. The residue was diluted with dichloromethane (500 mL). The solid was filtered out. The filtrate was concentrated under vacuum. The residue was purified by column chromatography on silica gel using 90% petroleum ether and 10% ethyl acetate as eluent to afford diethyl 1-benzyl-4,4-dimethyl-5-oxo-4,5-dihydro-1H-pyrrole-2,3-dicarboxylate (50 g, 80% yield) as a light-yellow oil. ¹H NMR (300 MHz, Chloroform-d) δ: 7.28-7.35 (m, 3H), 7.15-7.18 (m, 2H), 4.78 (s, 2H), 4.08-4.24 (m, 4H), 1.47 (s, 6H), 1.27 (t, J=7.2 Hz, 3H), 1.11 (t, J=7.2 Hz, 3H). LC-MS: m/z 346 [M+H]⁺.

### Step 2: 1-benzyl-4,4-dimethyl-5-oxo-4,5-dihydro-1H-pyrrole-2,3-dicarboxylic acid

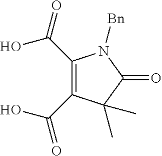 [SMILES: CC1(C)C(=O)N(Cc2ccccc2)C(C(=O)O)=C1C(=O)O]

To a stirred solution of diethyl 1-benzyl-4,4-dimethyl-5-oxo-4,5-dihydro-1H-pyrrole-2,3-dicarboxylate (36.0 g, 104.2 mmol) in methanol (240 mL) and water (110 mL) was added NaOH (12.5 g, 312.7 mmol) at 25° C. The reaction mixture was stirred at 60° C. for 16 h. The mixture was allowed to cool down to 25° C. The resulting mixture was concentrated under vacuum. The residue was diluted with water (500 mL). The pH was adjusted to 1-2 with HCl (4 M). The mixture was extracted with ethyl acetate (2×500 mL). The combined organic layers were dried over anhydrous sodium sulfate and concentrated under vacuum to give 1-benzyl-4,4-dimethyl-5-oxo-4,5-dihydro-1H-pyrrole-2,3-dicarboxylic acid (24.5 g, 77% yield) as a white solid. ¹H NMR (400 MHz, DMSO-d₆) δ: 7.20-7.40 (m, 3H), 7.12-7.14 (m, 2H), 4.70 (s, 2H), 1.28 (s, 6H). LC-MS: m/z 290 [M+H]⁺.

### Step 3: 1-benzyl-3,3-dimethyl-5,6-dihydro-1H-pyrrolo[2,3-d]pyridazine-2,4,7(3H)-trione

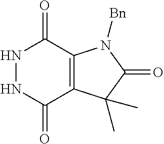 [SMILES: CC1(C)C(=O)N(Cc2ccccc2)c2c1c(=O)[nH][nH]c2=O]

To a stirred solution of 1-benzyl-4,4-dimethyl-5-oxo-4,5-dihydro-1H-pyrrole-2,3-dicarboxylic acid (5.0 g, 17.3 mmol) in ethyl acetate (300 mL) were added hydrazine monohydrochloride (2.4 g, 34.6 mmol), 1-propylphosphonic acid cyclic anhydride (22.0 g, 69.1 mmol, 50% in ethyl acetate) and DIEA (11.2 g, 86.4 mmol). The reaction mixture was stirred at 25° C. for 72 h. The mixture was quenched by the addition of water (500 mL). The resulting mixture was extracted with ethyl acetate (3×500 mL). The combined organic layers were dried over anhydrous sodium sulfate and concentrated under vacuum. This resulted in 1-benzyl-3,3-dimethyl-5,6-dihydro-1H-pyrrolo[2,3-d]pyridazine-2,4,7(3H)-trione (4.5 g, crude) as a white solid. ¹H NMR (400 MHz, DMSO-d₆) δ: 7.20-7.40 (m, 5H), 5.23 (s, 2H), 1.52 (s, 6H). LC-MS: m/z 286 [M+H]⁺.

### Step 4: 1-benzyl-4,7-dichloro-3,3-dimethyl-1,3-dihydro-2H-pyrrolo[2,3-d]pyridazin-2-one

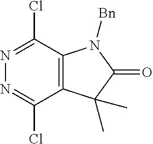 [SMILES: CC1(C)C(=O)N(Cc2ccccc2)c2c(Cl)nnc(Cl)c21]

A solution of 1-benzyl-3,3-dimethyl-5,6-dihydro-1H-pyrrolo[2,3-d]pyridazine-2,4,7(3H)-trione (11 g, 38.56 mmol) in phosphorus oxychloride (59.1 g, 385.6 mmol) was stirred at 90° C. for 16 h. The mixture was allowed to cool down to 25° C. The resulting mixture was concentrated under vacuum. The residue was diluted with ethyl acetate (500 mL). The organic layer was washed with saturated aqueous NaHCO₃ solution (500 mL), dried over anhydrous sodium sulfate and concentrated under vacuum. The residue was purified by column chromatography on silica gel using 85% petroleum ether and 15% ethyl acetate as eluent to afford 1-benzyl-4,7-dichloro-3,3-dimethyl-1,3-dihydro-2H-pyrrolo[2,3-d]pyridazin-2-one (8 g, 58% yield) as a white solid. ¹H NMR (400 MHz, DMSO-d₆) δ: 7.01-7.48 (m, 5H), 5.21 (s, 2H), 1.50 (s, 6H). LC-MS: m/z 322 [M+H]⁺.

### Step 5: 1-benzyl-4-chloro-7-hydrazineyl-3,3-dimethyl-1,3-dihydro-2H-pyrrolo[2,3-d]pyridazin-2-one

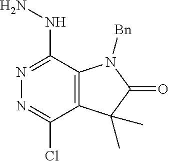 [SMILES: CC1(C)C(=O)N(Cc2ccccc2)c2c(NN)nnc(Cl)c21]

To a stirred solution of 1-benzyl-4,7-dichloro-3,3-dimethyl-1,3-dihydro-2H-pyrrolo[2,3-d]pyridazin-2-one (2.6 g, 8.1 mmol) in ethanol (20 mL) was added hydrazine hydrate (6.1 g, 121.1 mmol, 80%). The reaction mixture was stirred at 90° C. for 16 h. The mixture was allowed to cool down to 25° C. The resulting mixture was concentrated under vacuum. The residue was purified by column chromatography on silica gel using 90% dichloromethane and 10% methanol as eluents to afford 1-benzyl-4-chloro-7-hydrazineyl-3,3-dimethyl-1,3-dihydro-2H-pyrrolo[2,3-d]pyridazin-2-one (1.5 g, 53% yield) as a light-yellow solid. ¹H NMR (400 MHz, DMSO-d₆) δ: 7.41 (br, 1H), 7.07-7.34 (m, 5H), 5.22 (s, 2H), 4.39 (br, 2H), 1.43 (s, 6H). LC-MS: m/z 318 [M+H]⁺.

### Step 6: 1-benzyl-4-chloro-3,3-dimethyl-1,3-dihydro-2H-pyrrolo[2,3-d]pyridazin-2-one

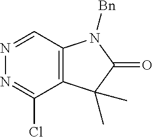 [SMILES: CC1(C)C(=O)N(Cc2ccccc2)c2cnnc(Cl)c21]

To a stirred solution of 1-benzyl-4-chloro-7-hydrazineyl-3,3-dimethyl-1,3-dihydro-2H-pyrrolo[2,3-d]pyridazin-2-one (800 mg, 2.5 mmol) in methanol (10 mL) and water (10 mL) was added CuSO₄ (2.0 g, 12.6 mmol). The reaction mixture was stirred at 25° C. for 2 h. The resulting mixture was concentrated under vacuum. The residue was diluted with ethyl acetate (50 mL). The organic layer was washed with brine (50 mL), dried over anhydrous sodium sulfate and concentrated under vacuum. The residue was purified by column chromatography on silica gel using 70% petroleum ether and 30% ethyl acetate as eluents to afford 1-benzyl-4-chloro-3,3-dimethyl-1,3-dihydro-2H-pyrrolo[2,3-d]pyridazin-2-one (500 mg, 62% yield) as a brown oil. ¹H NMR (300 MHz, DMSO-d₆) δ: 9.24 (s, 1H), 7.02-7.59 (m, 5H), 5.00 (s, 2H), 1.47 (s, 6H). LC-MS: m/z 288 [M+H]⁺.

### Step 7: 4-amino-1-benzyl-3,3-dimethyl-1,3-dihydro-2H-pyrrolo[2,3-d]pyridazin-2-one

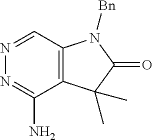 [SMILES: CC1(C)C(=O)N(Cc2ccccc2)c2cnnc(N)c21]

To a stirred solution of 1-benzyl-4-chloro-3,3-dimethyl-1,3-dihydro-2H-pyrrolo[2,3-d]pyridazin-2-one (1 g, 3.48 mmol) in ethanol (0.5 mL) was added ammonium hydroxide (30 mL). The reaction mixture was stirred at 150° C. for 72 h. The mixture was allowed to cool down to 25° C. The resulting mixture was concentrated under vacuum. The residue was purified by column chromatography on silica gel using 97% dichloromethane and 3% methanol as eluents to afford 4-amino-1-benzyl-3,3-dimethyl-1,3-dihydro-2H-pyrrolo[2,3-d]pyridazin-2-one (500 mg, 48% yield) as a light-yellow solid. ¹H NMR (400 MHz, DMSO-d₆) δ: 8.47 (d, J=1.6 Hz, 1H), 7.23-7.39 (m, 5H), 6.37 (s, 2H), 4.89 (s, 2H), 1.39 (s, 6H). LC-MS: m/z 269 [M+H]⁺.

### Step 8: 1-benzyl-3,3-dimethyl-2,3-dihydro-1H-pyrrolo[2,3-d]pyridazin-4-amine

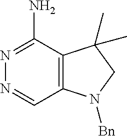 [SMILES: CC1(C)CN(Cc2ccccc2)c2cnnc(N)c21]

To a stirred solution of 4-amino-1-benzyl-3,3-dimethyl-1,3-dihydro-2H-pyrrolo[2,3-d]pyridazin-2-one (100 mg, 372.7 μmol) in tetrahydrofuran (3 mL) was added borane (3 mL, 3 mmol, 1 M in tetrahydrofuran). The reaction mixture was stirred at 25° C. for 24 h. The resulting mixture was concentrated under vacuum. The residue was purified by Prep-TLC using 95% dichloromethane and 5% methanol as eluents to afford 1-benzyl-3,3-dimethyl-2,3-dihydro-1H-pyrrolo[2,3-d]pyridazin-4-amine (28 mg, 26% yield) as a colorless oil. LC-MS: m/z 255 [M+H]⁺.

### Step 9: 7-benzyl-2,9,9-trimethyl-8,9-dihydro-7H-imidazo[1,2-b]pyrrolo[3,2-d]pyridazine

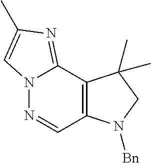

To a stirred solution of 1-benzyl-3,3-dimethyl-2,3-dihydro-1H-pyrrolo[2,3-d]pyridazin-4-amine (50 mg, 196.6 μmol) in dichloromethane (4 mL) was added bromoacetone (269 mg, 2.0 mmol) at 0° C. under nitrogen atmosphere. The reaction mixture was stirred at 40° C. for 16 h. The resulting mixture was concentrated under vacuum. The residue was purified by Prep-TLC using 90% dichloromethane and 10% methanol as eluents to afford 7-benzyl-2,9,9-trimethyl-8,9-dihydro-7H-imidazo[1,2-b]pyrrolo[3,2-d]pyridazine (14 mg, 22% yield) as a brown oil. ¹H NMR (300 MHz, Chloroform-d) δ: 7.90 (s, 1H), 7.56 (q, J=0.9 Hz, 1H), 7.29-7.45 (m, 5H), 4.37 (s, 2H), 3.25 (s, 2H), 2.49 (d, J=0.9 Hz, 3H), 1.60 (s, 6H). LC-MS: m/z 293 [M+H]⁺.

### Step 10: 2,9,9-trimethyl-8,9-dihydro-7H-imidazo[1,2-b]pyrrolo[3,2-d]pyridazine

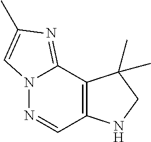

To a stirred solution of 7-benzyl-2,9,9-trimethyl-8,9-dihydro-7H-imidazo[1,2-b]pyrrolo[3,2-d]pyridazine (30 mg, 102.6 μmol) in methanol (5 mL) was added Pd/C (100 mg) and HCl (240 μL, 1 M). The reaction mixture was stirred at 25° C. for 3 h under hydrogen atmosphere. The solid was filtered out. The filtrate was concentrated under vacuum. The residue was purified by Prep-TLC using 90% dichloromethane and 10% methanol as eluents to afford 2,9,9-trimethyl-8,9-dihydro-7H-imidazo[1,2-b]pyrrolo[3,2-d]pyridazine (15 mg, 65% yield) as a brown oil. LC-MS: m/z 203 [M+H]⁺.

### Step 11: N-(5-chloro-6-(2H-1,2,3-triazol-2-yl)pyridin-3-yl)-2,9,9-trimethyl-8,9-dihydro-7H-imidazo[1,2-b]pyrrolo[3,2-d]pyridazine-7-carboxamide

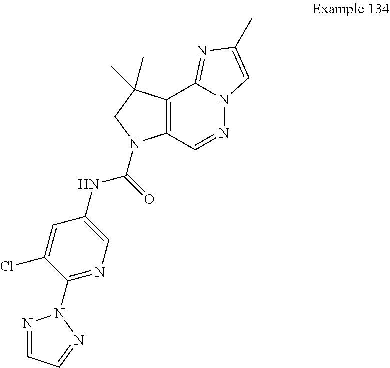 [SMILES: Cc1cn2ncc3c(c2n1)C(C)(C)CN3C(=O)Nc1cnc(-n2nccn2)c(Cl)c1]

To a solution of 5-chloro-6-(2H-1,2,3-triazol-2-yl)pyridin-3-amine (Method A1 step 2; 10 mg, 51.9 μmol) in tetrahydrofuran (2 mL) were added triphosgene (13 mg, 44.5 μmol) and TEA (11 mg, 111.2 μmol). The mixture was stirred at 25° C. for 0.5 h and then filtered. The filtrate was added to a solution of 2,9,9-trimethyl-8,9-dihydro-7H-imidazo[1,2-b]pyrrolo[3,2-d]pyridazine (15 mg, 74.2 μmol) in tetrahydrofuran (1 mL). To this solution were added TEA (75 mg, 741.6 μmol) and N,N-dimethylpyridin-4-amine (18 mg, 148.3 μmol). The reaction mixture was stirred at 40° C. for 2 h. The resulting mixture was concentrated under vacuum. The residue was purified by Prep-TLC using 95% dichloromethane and 5% methanol as eluents to afford 15 mg of crude product. The afforded crude product was purified by Prep-HPLC and the collected fractions were lyophilized to afford N-(5-chloro-6-(2H-1,2,3-triazol-2-yl)pyridin-3-yl)-2,9,9-trimethyl-8,9-dihydro-7H-imidazo[1,2-b]pyrrolo[3,2-d]pyridazine-7-carboxamide (6.4 mg, 20% yield) as a white solid.

Example 134: ¹H NMR (300 MHz, DMSO-d₆) δ: 9.47 (s, 1H), 9.16 (s, 1H), 8.79 (d, J=2.3 Hz, 1H), 8.55 (d, J=2.4 Hz, 1H), 8.18 (s, 2H), 7.99 (d, J=0.9 Hz, 1H), 4.13 (s, 2H), 2.39 (d, J=0.9 Hz, 3H), 1.63 (s, 6H). LC-MS: m/z 424 [M+H]⁺.

### Method T4

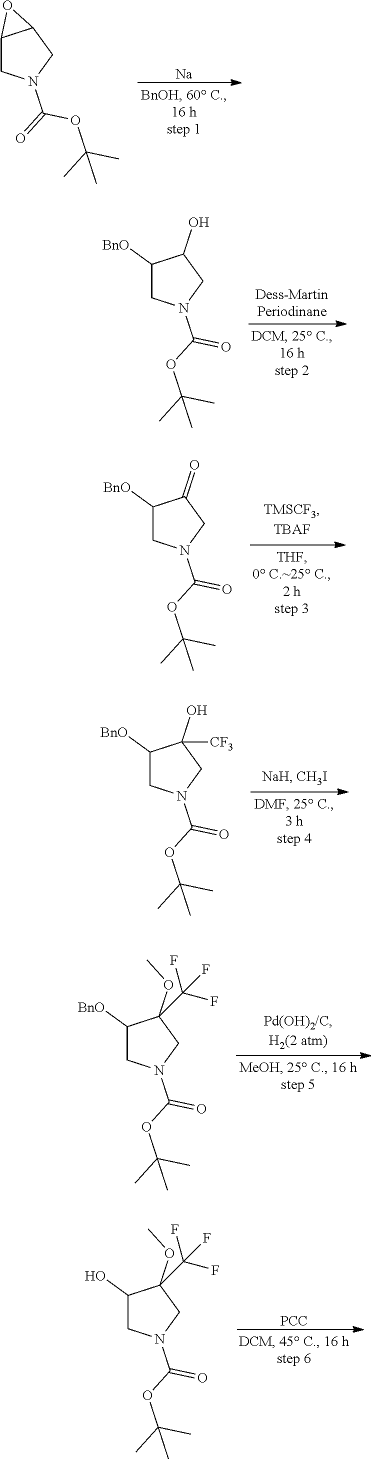

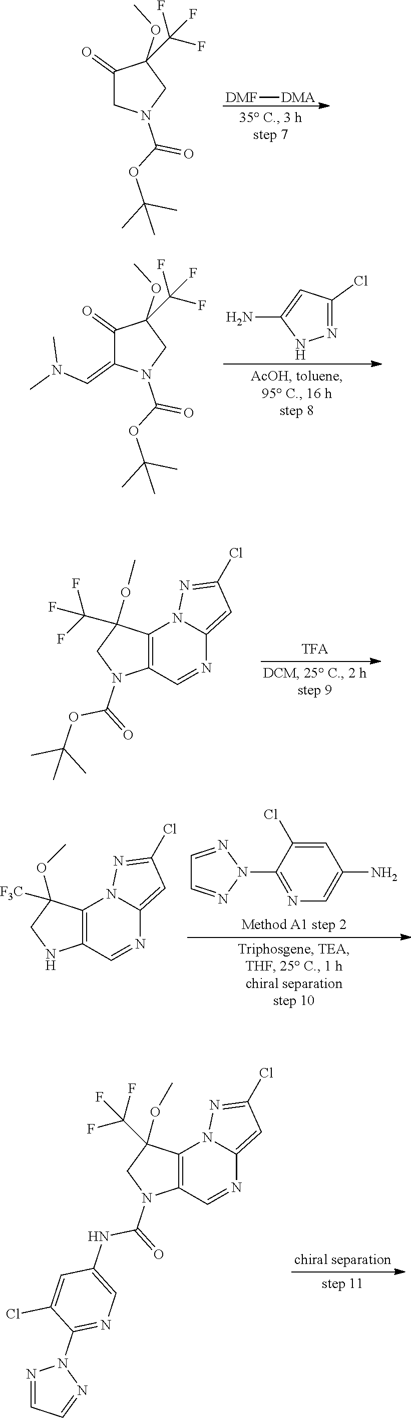

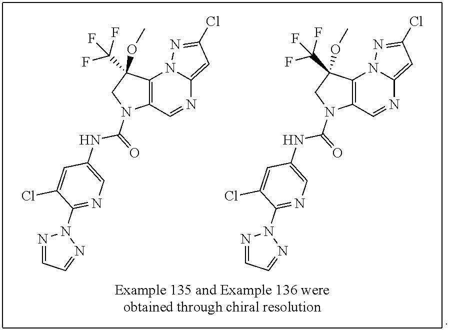 [SMILES: CO[C@]1(C(F)(F)F)CN(C(=O)Nc2cnc(-n3nccn3)c(Cl)c2)c2cnc3cc(Cl)nn3c21 ; CO[C@@]1(C(F)(F)F)CN(C(=O)Nc2cnc(-n3nccn3)c(Cl)c2)c2cnc3cc(Cl)nn3c21]

### Examples 135 and 136: Single enantiomers obtained from a racemic mixture containing (R)-2-chloro-N-(5-chloro-6-(2H-1,2,3-triazol-2-yl)pyridin-3-yl)-8-methoxy-8-(trifluoromethyl)-7,8-dihydro-6H-pyrazolo[1,5-a]pyrrolo[2,3-e]pyrimidine-6-carboxamide and (S)-2-chloro-N-(5-chloro 2H-1,2,3-triazol-2-yl)pyridin-3-yl)-8-methoxy-8-(trifluoromethyl)-7,8-dihydro-6H-pyrazolo[1,5-a]pyrrolo[2,3-e]pyrimidine-6-carboxamide

### Step 1: tert-butyl 3-(benzyloxy)-4-hydroxypyrrolidine-1-carboxylate

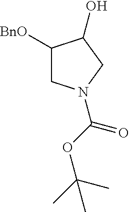 [SMILES: CC(C)(C)OC(=O)N1CC(O)C(OCc2ccccc2)C1]

Sodium (9.3 g, 404.9 mmol) was added in phenylmethanol (146.0 g, 1.4 mol) at 0° C. in portions. The reaction was stirred at 60° C. for 40 min. Then the reaction was added tert-butyl 6-oxa-3-azabicyclo[3.1.0]hexane-3-carboxylate (50.0 g, 270.0 mmol). The resulting mixture was stirred at 60° C. for 16 h. The mixture was allowed to cool down to 25° C. The reaction mixture was quenched by the addition of water (500 mL). The resulting solution was extracted with ethyl acetate (3×500 mL). The combined organic layers were dried over anhydrous sodium sulfate and concentrated under vacuum. The residue was purified by column chromatography on silica gel using 70% petroleum ether and 30% ethyl acetate as eluents to afford tert-butyl 3-(benzyloxy)-4-hydroxypyrrolidine-1-carboxylate (40 g, 40% yield) as a yellow oil. ¹H NMR (300 MHz, DMSO-d₆) δ: 7.26-7.39 (m, 5H), 5.22 (d, J=3 Hz, 1H), 4.55 (d, J=3 Hz, 2H), 4.14 (s, 1H), 3.82 (s, 1H), 3.37-3.45 (m, 2H), 3.16-3.20 (m, 2H), 1.40 (s, 9H); LC-MS: m/z 294 [M+H]⁺.

### Step 2: tert-butyl 3-(benzyloxy)-4-oxopyrrolidine-1-carboxylate

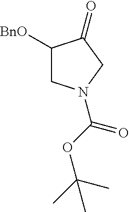 [SMILES: CC(C)(C)OC(=O)N1CC(=O)C(OCc2ccccc2)C1]

To a solution of tert-butyl 3-(benzyloxy)-4-hydroxypyrrolidine-1-carboxylate (40.0 g, 136.5 mmol) in dichloromethane (800 mL) was added Dess-Martin Periodinane (86.8 g, 240.8 mmol). The resulting mixture was stirred at 25° C. for 16 h. The solid was filtered out. The filtrate was added dichloromethane (400 mL), washed with saturated aqueous NaHSO₃ solution (400 mL) and saturated aqueous NaHCO₃ solution (400 mL). The combined organic layers were dried over anhydrous sodium sulfate and concentrated under vacuum. The residue was purified by column chromatography using 80% petroleum ether and 20% ethyl acetate as eluents to afford tert-butyl 3-(benzyloxy)-4-oxopyrrolidine-1-carboxylate (30 g, 75% yield) as a yellow oil. LC-MS: m/z 292 [M+H]⁺.

### Step 3: tert-butyl 4-(benzyloxy)-3-hydroxy-3-(trifluoromethyl)pyrrolidine-1-carboxylate

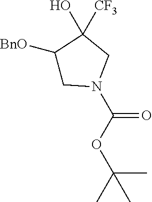 [SMILES: CC(C)(C)OC(=O)N1CC(OCc2ccccc2)C(O)(C(F)(F)F)C1]

To a solution of tert-butyl 3-(benzyloxy)-4-oxopyrrolidine-1-carboxylate (30 g, 102.7 mmol) in tetrahydrofuran (800 mL) was added (trifluoromethyl)trimethylsilane (72.9 g, 513.5 mmol). To this solution was added tetrabutylammonium fluoride (205 mL, 205 mmol, 1 M in THF) at 0° C. The resulting mixture was stirred at 25° C. for 2 h. The reaction mixture was quenched by the addition of water (500 mL). The resulting solution was extracted with ethyl acetate (3×500 mL). The combined organic layers were washed by saturated aqueous NH₄Cl solution (3×500 mL), dried over anhydrous sodium sulfate and concentrated under vacuum. The residue was purified by column chromatography on silica gel using 75% petroleum ether and 25% ethyl acetate as eluents to afford tert-butyl 4-(benzyloxy)-3-hydroxy-3-(trifluoromethyl)pyrrolidine-1-carboxylate (20 g, 54% yield) as a yellow solid. ¹H NMR (300 MHz, DMSO-d₆) δ: 7.27-7.37 (m, 5H), 6.45 (s, 1H), 4.55-4.65 (m, 2H), 4.18-4.23 (m, 1H), 3.67-3.73 (m, 1H), 3.47-3.51 (m, 1H), 3.36 (d, J=12 Hz, 1H), 3.19-3.22 (m, 1H), 1.39 (s, 9H); LC-MS: m/z 362 [M+H]⁺.

### Step 4: tert-butyl 4-(benzyloxy)-3-methoxy-3-(trifluoromethyl)pyrrolidine-1-carboxylate

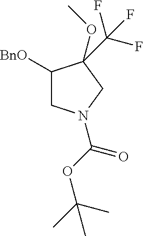 [SMILES: COC1(C(F)(F)F)CN(C(=O)OC(C)(C)C)CC1OCc1ccccc1]

To a solution of tert-butyl 4-(benzyloxy)-3-hydroxy-3-(trifluoromethyl)pyrrolidine-1-carboxylate (20.0 g, 55.4 mmol) in N,N-Dimethylformamide (320 mL) were added NaH (4.4 g, 110.8 mmol, 60% in mineral oil) at 0° C. The reaction mixture was stirred at 0° C. for 0.5 h. To the mixture was then added iodomethane (23.6 g, 166.2 mmol) at 0° C. The resulting mixture was stirred at 25° C. for 3 h. The reaction mixture was quenched by the addition of water (500 mL). The resulting solution was extracted with ethyl acetate (500 mL) and washed by brine (3×500 mL). The organic layer was dried over anhydrous sodium sulfate and concentrated under vacuum. The residue was purified by column chromatography on silica gel using 75% petroleum ether and 25% ethyl acetate as eluents to afford tert-butyl 4-(benzyloxy)-3-methoxy-3-(trifluoromethyl)pyrrolidine-1-carboxylate (15.6 g, 75% yield) as a yellow oil. ¹H NMR (300 MHz, DMSO-d₆) δ: 6.48-6.56 (m, 5H), 4.03 (s, 2H), 3.77-3.89 (m, 2H), 3.53-3.58 (m, 1H), 2.86-3.01 (m, 2H), 2.74 (s, 3H), 0.66 (s, 9H); LC-MS: m/z 376 [M+H]⁺.

### Step 5: tert-butyl 4-hydroxy-3-methoxy-3-(trifluoromethyl)pyrrolidine-1-carboxylate

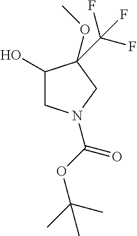 [SMILES: COC1(C(F)(F)F)CN(C(=O)OC(C)(C)C)CC1O]

To a solution of tert-butyl 4-(benzyloxy)-3-methoxy-3-(trifluoromethyl)pyrrolidine-1-carboxylate (15.6 g, 41.6 mmol) in methanol (300 mL) was added Pd(OH)₂/C (7.8 g, 50%). The flask was evacuated and flushed three times with nitrogen, followed by flushing with hydrogen. The mixture was stirred for 16 h at 25° C. under an atmosphere of hydrogen balloon. The solid was filtered out. The filtrate was concentrated under vacuum. The residue was purified by column chromatography on silica gel using 75% petroleum ether and 25% ethyl acetate as eluents to afford tert-butyl 4-hydroxy-3-methoxy-3-(trifluoromethyl)pyrrolidine-1-carboxylate (8.0 g, 67% yield) as a white solid. ¹H NMR (300 MHz, DMSO-d₆) δ: 5.55 (s, 1H), 4.36 (d, J=8 Hz, 1H), 3.68-3.73 (m, 1H), 3.52-3.58 (m, 1H), 3.42 (s, 3H), 3.36-3.39 (m, 1H), 3.06-3.12 (m, 1H), 1.38 (s, 9H); LC-MS: m/z 286 [M+H]⁺.

### Step 6: tert-butyl 3-methoxy-4-oxo-3-(trifluoromethyl)pyrrolidine-1-carboxylate

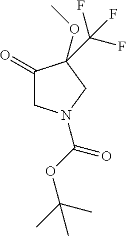 [SMILES: COC1(C(F)(F)F)CN(C(=O)OC(C)(C)C)CC1=O]

To a solution of tert-butyl 4-hydroxy-3-methoxy-3-(trifluoromethyl)pyrrolidine-1-carboxylate (5 g, 17.5 mmol) in dichloromethane (200 mL) were added PCC (37.6 g, 175 mmol) and SiO₂ (37.6 g). The resulting mixture was stirred at 45° C. for 16 h. The mixture was allowed to cool down to 25° C. The solid was filtered out. The resulting solution was concentrated under vacuum. The residue was purified by column chromatography on silica gel using 75% petroleum ether and 25% ethyl acetate as eluents to afford tert-butyl 3-methoxy-4-oxo-3-(trifluoromethyl)pyrrolidine-1-carboxylate (1.5 g, 50% yield) as a white solid. ¹H NMR (300 MHz, DMSO-d₆) δ: 4.02-4.06 (m, 4H), 3.39 (s, 3H), 1.42 (s, 9H); LC-MS: m/z 284 [M+H]⁺.

### Step 7: tert-butyl (E)-2-((dimethylamino)methylene)-4-methoxy-3-oxo-4-(trifluoromethyl)pyrrolidine-1-carboxylate

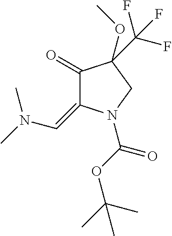 [SMILES: COC1(C(F)(F)F)CN(C(=O)OC(C)(C)C)/C(=C/N(C)C)C1=O]

A solution of tert-butyl 3-methoxy-4-oxo-3-(trifluoromethyl)pyrrolidine-1-carboxylate (1.5 g, 5.2 mmol) in DMF-DMA (30 mL) was stirred at 35° C. for 3 h. The mixture was allowed to cool down to 25° C. The resulting solution was concentrated under vacuum to give tert-butyl (E)-2-((dimethylamino)methylene)-4-methoxy-3-oxo-4-(trifluoromethyl)pyrrolidine-1-carboxylate (1.6 g, crude) as a yellow oil which was used to the next step directly. LC-MS: m/z 339 [M+H]⁺.

### Step 8: tert-butyl 2-chloro-8-methoxy-8-(trifluoromethyl)-7,8-dihydro-6H-pyrazolo[1,5-a]pyrrolo[2,3-e]pyrimidine-6-carboxylate

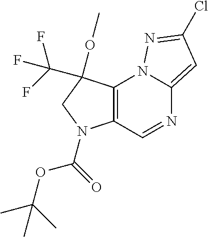 [SMILES: COC1(C(F)(F)F)CN(C(=O)OC(C)(C)C)c2cnc3cc(Cl)nn3c21]

To a solution of tert-butyl (E)-2-((dimethylamino)methylene)-4-methoxy-3-oxo-4-(trifluoromethyl)pyrrolidine-1-carboxylate (1.6 g, 4.7 mmol) in toluene (30 mL) were added 3-chloro-1H-pyrazol-5-amine (550 mg, 4.7 mmol) and acetic acid (3 mL). The resulting mixture was stirred at 95° C. for 16 h. The mixture was allowed to cool down to 25° C. The resulting solution was concentrated under vacuum. The residue was purified by column chromatography on silica gel using 75% petroleum ether and 25% ethyl acetate as eluents to afford tert-butyl 2-chloro-8-methoxy-8-(trifluoromethyl)-7,8-dihydro-6H-pyrazolo[1,5-a]pyrrolo[2,3-e]pyrimidine-6-carboxylate (265 mg, 14% yield) as a yellow oil. ¹H NMR (300 MHz, DMSO-d₆) δ: 9.16 (s, 1H), 7.15 (s, 1H), 4.20-4.41 (m, 2H), 3.26 (s, 3H), 1.57 (s, 9H); LC-MS: m/z 393 [M+H]⁺.

### Step 9: 2-chloro-8-methoxy-8-(trifluoromethyl)-7,8-dihydro-6H-pyrazolo[1,5-a]pyrrolo[2,3-e]pyrimidine

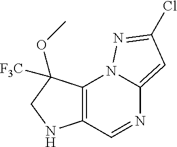 [SMILES: COC1(C(F)(F)F)CNc2cnc3cc(Cl)nn3c21]

To a solution of tert-butyl 2-chloro-8-methoxy-8-(trifluoromethyl)-7,8-dihydro-6H-pyrazolo[1,5-a]pyrrolo[2,3-e]pyrimidine-6-carboxylate (265 mg, 676.0 μmol) in dichloromethane (20 mL) was added TFA (5 mL). The resulting mixture was stirred for 2 h at 25° C. The mixture was concentrated under vacuum. The pH was adjusted to 7-8 with saturated aqueous NaHCO₃ solution. The resulting mixture was extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate as eluents to afford 2-chloro-8-methoxy-8-(trifluoromethyl)-7,8-dihydro-6H-pyrazolo[1,5-a]pyrrolo[2,3-e]pyrimidine (100 mg, 50% yield) as a yellow oil. LC-MS: m/z 293 [M+H]⁺.

### Step 10: 2-chloro-N-(5-chloro-6-(2H-1,2,3-triazol-2-yl)pyridin-3-yl)-8-methoxy-8-(trifluoromethyl)-7,8-dihydro-6H-pyrazolo[1,5-a]pyrrolo[2,3-e]pyrimidine-6-carboxamide

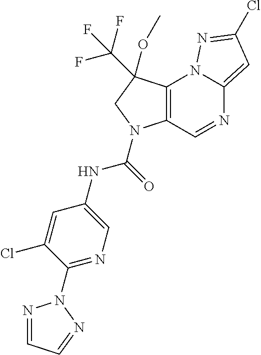 [SMILES: COC1(C(F)(F)F)CN(C(=O)Nc2cnc(-n3nccn3)c(Cl)c2)c2cnc3cc(Cl)nn3c21]

To a stirred mixture of 5-chloro-6-(triazol-2-yl)pyridin-3-amine (Method A1 step 2; 100 mg, 512.8 umol) in tetrahydrofuran (20 mL) was added triphosgene (61 mg, 205.4 umol) and TEA (104 mg, 1.0 mmol). The resulting mixture was stirred at 25° C. for 1 h and then filtered. The resulting filtrate was added to a solution of 2-chloro-8-methoxy-8-(trifluoromethyl)-7,8-dihydro-6H-pyrazolo[1,5-a]pyrrolo[2,3-e]pyrimidine (100 mg, 342.4 umol) in tetrahydrofuran (1 mL). The mixture was stirred at 25° C. for 1 h. The reaction mixture was quenched by the addition of water (50 mL). The resulting solution was extracted with ethyl acetate (3×50 mL). The combined organic layers were dried over anhydrous sodium sulfate and concentrated under vacuum. The residue was purified by column chromatography on silica gel using 10% methanol and 90% dichloromethane as eluents to afford the crude product which was purified by Prep-HPLC and the collected fractions were lyophilized to give 2-chloro-N-(5-chloro-6-(2H-1,2,3-triazol-2-yl)pyridin-3-yl)-8-methoxy-8-(trifluoromethyl)-7,8-dihydro-6H-pyrazolo[1,5-a]pyrrolo[2,3-e]pyrimidine-6-carboxamide (70 mg, 40% yield) as a white solid. LC-MS: m/z 514 [M+H]⁺.

### Step 11: Separation of enantiomers to obtain (S)-2-chloro-N-(5-chloro-6-(2H-1,2,3-triazol-2-yl)pyridin-3-yl)-8-methoxy-8-(trifluoromethyl)-7,8-dihydro-6H-pyrazolo[1,5-a]pyrrolo[2,3-e]pyrimidine-6-carboxamide and (R)-2-chloro-N-(5-chloro-6-(2H-1,2,3-triazol-2-yl)pyridin-3-yl)-8-methoxy-8-(trifluoromethyl)-7,8-dihydro-6H-pyrazolo[1,5-a]pyrrolo[2,3-e]pyrimidine-6-carboxamide

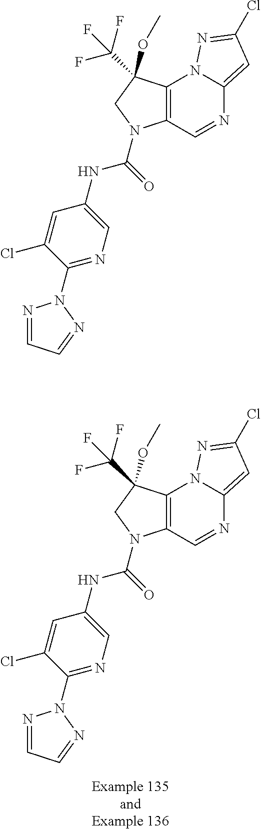 [SMILES: CO[C@]1(C(F)(F)F)CN(C(=O)Nc2cnc(-n3nccn3)c(Cl)c2)c2cnc3cc(Cl)nn3c21 ; CO[C@@]1(C(F)(F)F)CN(C(=O)Nc2cnc(-n3nccn3)c(Cl)c2)c2cnc3cc(Cl)nn3c21]

70 mg of 2-chloro-N-(5-chloro-6-(2H-1,2,3-triazol-2-yl)pyridin-3-yl)-8-methoxy-8-(trifluoromethyl)-7,8-dihydro-6H-pyrazolo[1,5-a]pyrrolo[2,3-e]pyrimidine-6-carboxamide was submitted to chiral HPLC purification (Column: CHIRAL ART Cellulose-SC, 2×25 cm, 5 um; Mobile Phase A:Hex:DCM=3:1(0.5% 2M NH₃-MeOH)—HPLC, Mobile Phase B:EtOH-HPLC; Flow rate: 20 mL/min; Gradient: 50 B to 50 B in 14 min; 220/254 nm; RT1:7.66; RT2:11.725; Injection Volume: 3 ml; Number Of Runs: 3; The first eluting isomer was concentrated and lyophilized to afford Example 135 (24.5 mg, 13% yield) as an off-white solid. The second eluting isomer was concentrated and lyophilized to afford Example 136 (26 mg, 14% yield) as an off-white solid. Examples 135 and 136 are enantiomers, but their absolute stereochemistry is not yet known.

Example 135: ¹H NMR (400 MHz, DMSO-d₆) δ: 9.82 (s, 1H), 9.55 (s, 1H), 8.82 (d, J=2.4 Hz, 1H), 8.58 (d, J=2.4 Hz, 1H), 8.24 (s, 2H), 7.23 (s, 1H), 4.74-4.82 (m, 2H), 3.37 (s, 3H); LC-MS: m/z 514 [M+H]⁺.

Example 136: ¹H NMR (400 MHz, DMSO-d₆) δ: 9.75 (s, 1H), 9.49 (s, 1H), 8.76 (d, J=2.4 Hz, 1H), 8.52 (d, J=2.4 Hz, 1H), 8.18 (s, 2H), 7.16 (s, 1H), 4.72-4.73 (m, 2H), 3.31 (s, 3H); LC-MS: m/z 514 [M+H]⁺.

### Method U4

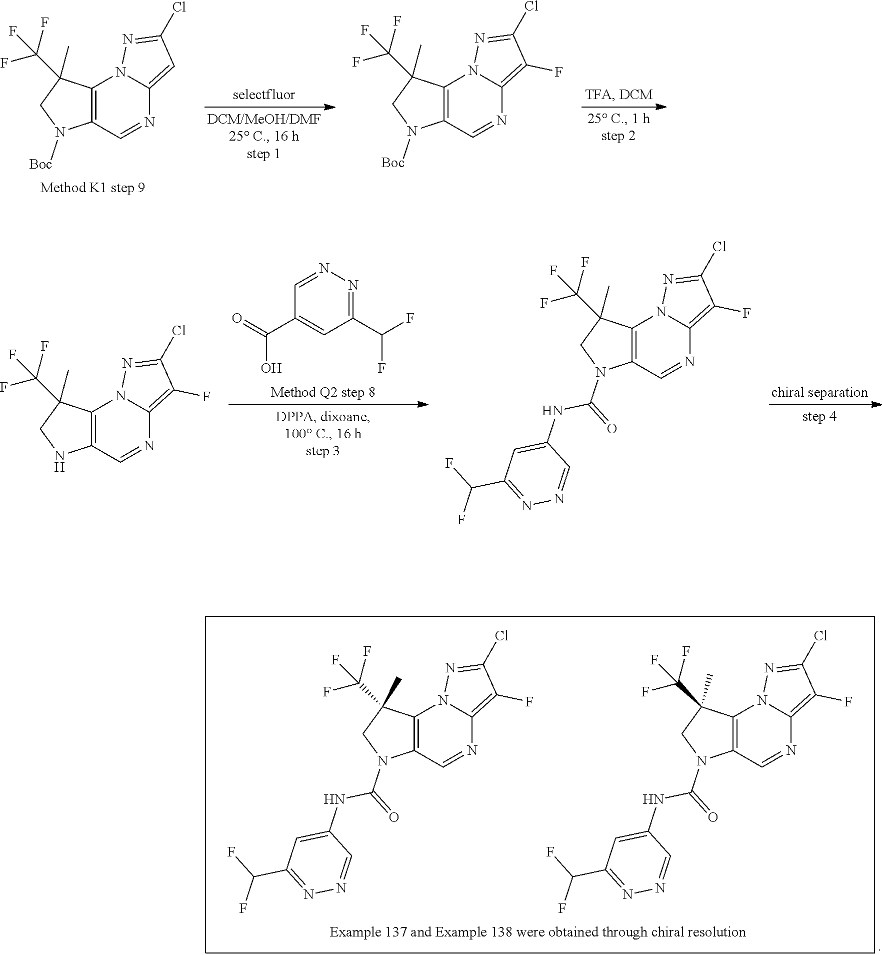

### Examples 136 and 137: Single enantiomers obtained from a racemic mixture containing (S)-2-chloro-N-(6-(difluoromethyl)pyridazin-4-yl)-3-fluoro-8-methyl-8-(trifluoromethyl)-7,8-dihydro-6H-pyrazolo[1,5-a]pyrrolo[2,3-e]pyrimidine-6-carboxamide and (R)-2-chloro-N-(6-(difluoromethyl)pyridazin-4-yl)-3-fluoro-8-methyl-8-(trifluoromethyl)-7,8-dihydro-6H-pyrazolo[1,5-a]pyrrolo[2,3-e]pyrimidine-6-carboxamide

### Step 1: tert-butyl 2-chloro-3-fluoro-8-methyl-8-(trifluoromethyl)-7,8-dihydro-6H-pyrazolo[1,5-a]pyrrolo[2,3-e]pyrimidine-6-carboxylate

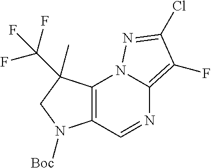 [SMILES: CC(C)(C)OC(=O)N1CC(C)(C(F)(F)F)c2c1cnc1c(F)c(Cl)nn21]

To a stirred solution of tert-butyl 2-chloro-8-methyl-8-(trifluoromethyl)-7,8-dihydro-6H-pyrazolo[1,5-a]pyrrolo[2,3-e]pyrimidine-6-carboxylate (Method K1 step 9; 300 mg, 797.4 μmol) in methanol (6 mL) and dichloromethane (6 mL) was added 1-Chloromethyl-4-fluoro-1,4-diazoniabicyclo[2.2.2]octane bis(tetrafluoroborate) (562 mg, 1.6 mmol) in N,N-dimethylformamide (2 mL) under nitrogen atmosphere at −20° C. The reaction mixture was stirred at 25° C. for 16 h. The reaction mixture was quenched with water (50 mL). The resulting mixture was extracted with ethyl acetate (3×50 mL). The combined organic layers were dried over anhydrous sodium sulfate and concentrated under vacuum. The residue was purified by column chromatography on silica gel using 80% petroleum ether and 20% ethyl acetate as eluents to afford tert-butyl 2-chloro-3-fluoro-8-methyl-8-(trifluoromethyl)-7,8-dihydro-6H-pyrazolo[1,5-a]pyrrolo[2,3-e]pyrimidine-6-carboxylate (110 mg, 35% yield) as a yellow solid. ¹H NMR (400 MHz, DMSO-d₆) δ: 9.21 (s, 1H), 4.01-4.37 (m, 2H), 1.52 (s, 9H), 1.23 (s, 3H). LC-MS: m/z 395 [M+H]⁺.

### Step 2: 2-chloro-3-fluoro-8-methyl-8-(trifluoromethyl)-7,8-dihydro-6H-pyrazolo[1,5-a]pyrrolo[2,3-e]pyrimidine

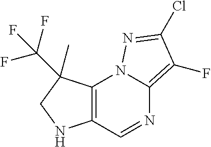 [SMILES: CC1(C(F)(F)F)CNc2cnc3c(F)c(Cl)nn3c21]

To a stirred solution of tert-butyl 2-chloro-3-fluoro-8-methyl-8-(trifluoromethyl)-7,8-dihydro-6H-pyrazolo[1,5-a]pyrrolo[2,3-e]pyrimidine-6-carboxylate (110 mg, 279.2 μmol) in dichloromethane (8 mL) was added TFA (2 mL). The reaction mixture was stirred at 25° C. for 1 h. The resulting mixture was concentrated under vacuum. The residue was diluted with saturated aqueous NaHCO₃ solution (40 mL). The resulting solution was extracted with dichloromethane (3×30 mL). The combined organic layers were washed with brine (30 mL), dried over anhydrous sodium sulfate and concentrated under vacuum. The residue was purified by column chromatography on silica gel using 50% petroleum ether and 50% ethyl acetate as eluents to afford 2-chloro-3-fluoro-8-methyl-8-(trifluoromethyl)-7,8-dihydro-6H-pyrazolo[1,5-a]pyrrolo[2,3-e]pyrimidine (50 mg, 61% yield) as a white solid. ¹H NMR (400 MHz, DMSO-d₆) δ: 8.39 (s, 1H), 6.12 (br, 1H), 3.85-3.95 (m, 1H), 3.52-3.62 (m, 1H), 1.77 (s, 3H). LC-MS: m/z 295 [M+H]⁺.

### Step 3: 2-chloro-N-(6-(difluoromethyl)pyridazin-4-yl)-3-fluoro-8-methyl-8-(trifluoromethyl)-7,8-dihydro-6H-pyrazolo[1,5-a]pyrrolo[2,3-e]pyrimidine-6-carboxamide

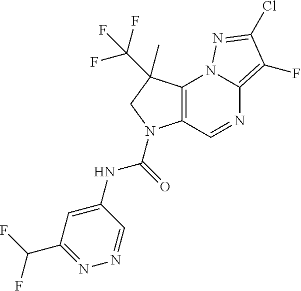 [SMILES: CC1(C(F)(F)F)CN(C(=O)Nc2cnnc(C(F)F)c2)c2cnc3c(F)c(Cl)nn3c21]

To a stirred solution of 2-chloro-3-fluoro-8-methyl-8-(trifluoromethyl)-7,8-dihydro-6H-pyrazolo[1,5-a]pyrrolo[2,3-e]pyrimidine (50 mg, 170.0 μmol) and 6-(difluoromethyl)pyridazine-4-carboxylic acid (Method Q2 step 8; 30 mg, 170.0 μmol) in dioxane (10 mL) was added DPPA (56 mg, 204.0 μmol) and TEA (86 mg, 850 μmol) at 25° C. The resulting mixture was stirred at 100° C. for 16 h. The reaction was cooled to 25° C. The resulting mixture was concentrated under vacuum. The residue was purified by Prep-TLC using 50% petroleum ether and 50% ethyl acetate as eluents to afford 40 mg of the crude product which was submitted to Prep-HPLC purification and the collected fractions were lyophilized to afford 2-chloro-N-(6-(difluoromethyl)pyridazin-4-yl)-3-fluoro-8-methyl-8-(trifluoromethyl)-7,8-dihydro-6H-pyrazolo[1,5-a]pyrrolo[2,3-e]pyrimidine-6-carboxamide (18 mg, 18% yield) as a yellow solid. ¹H NMR (400 MHz, DMSO-d₆) δ): 9.94 (br, 1H), 9.48 (d, J=2.4 Hz, 1H), 9.36 (s, 1H), 8.19 (d, J=2.4 Hz, 1H), 7.24 (t, J=54.2 Hz, 1H), 4.86 (d, J=11.6 Hz, 1H), 4.30 (d, J=11.6 Hz, 1H), 1.95 (s, 3H). LC-MS: m/z 466 [M+H]⁺.

### Step 4: Separation of enantiomers to obtain (S)-2-chloro-N-(6-(difluoromethyl)pyridazin-4-yl)-3-fluoro-8-methyl-8-(trifluoromethyl)-7,8-dihydro-6H-pyrazolo[1,5-a]pyrrolo[2,3-e]pyrimidine-6-carboxamide and (R)-2-chloro-N-(6-(difluoromethyl)pyridazin-4-yl)-3-fluoro-8-methyl-8-(trifluoromethyl)-7,8-dihydro-6H-pyrazolo[1,5-a]pyrrolo[2,3-e]pyrimidine-6-carboxamide

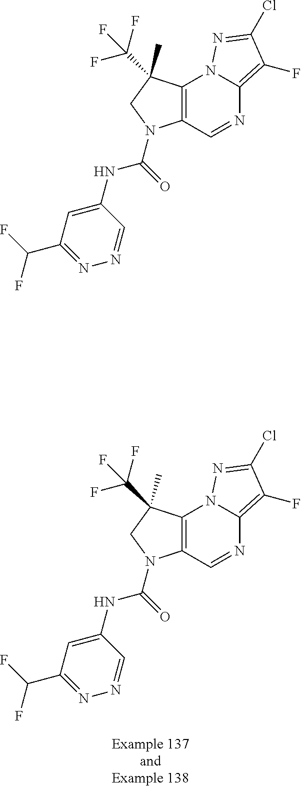 [SMILES: C[C@]1(C(F)(F)F)CN(C(=O)Nc2cnnc(C(F)F)c2)c2cnc3c(F)c(Cl)nn3c21 ; C[C@@]1(C(F)(F)F)CN(C(=O)Nc2cnnc(C(F)F)c2)c2cnc3c(F)c(Cl)nn3c21]

2-chloro-N-(6-(difluoromethyl)pyridazin-4-yl)-3-fluoro-8-methyl-8-(trifluoromethyl)-7,8-dihydro-6H-pyrazolo[1,5-a]pyrrolo[2,3-e]pyrimidine-6-carboxamide (16 mg, 34.5 μmol) was submitted to chiral-HPLC: Column: CHIRALPAK IE-3, 4.6×50 mm 3 um; Mobile Phase A: Hex (0.1% DEA):Ethanol=80: 20, Mobile Phase B: IPA-HPLC; Flow rate: 1 mL/min; Gradient: 0 B to 40 B in 10 min; 220/254 nm; RT1: 2.77; RT2: 3.06; Number Of Runs: 5. The first eluting isomer was concentrated and lyophilized to afford Example 137 (6 mg, 75% yield) as a white solid. The second eluting isomer was concentrated and lyophilized to afford Example 138 (4 mg, 44% yield) as a white solid. Examples 137 and 138 are enantiomers, but their absolute stereochemistry is not yet known.

Example 137: ¹H NMR (400 MHz, DMSO-d₆) δ: 9.94 (br, 1H), 9.49 (d, J=2.4 Hz, 1H), 9.36 (s, 1H), 8.20 (d, J=2.4 Hz, 1H), 7.24 (t, J=54.2 Hz, 1H), 4.86 (d, J=11.6 Hz, 1H), 4.30 (d, J=11.6 Hz, 1H), 1.95 (s, 3H). LC-MS: m/z 466 [M+H]⁺.

Example 138: ¹H NMR (400 MHz, DMSO-d₆) δ: 9.94 (br, 1H), 9.49 (d, J=2.4 Hz, 1H), 9.36 (s, 1H), 8.20 (d, J=2.4 Hz, 1H), 7.24 (t, J=54.2 Hz, 1H), 4.86 (d, J=11.6 Hz, 1H), 4.30 (d, J=11.6 Hz, 1H), 1.95 (s, 3H). LC-MS: m/z 466 [M+H]⁺.

### Method V4

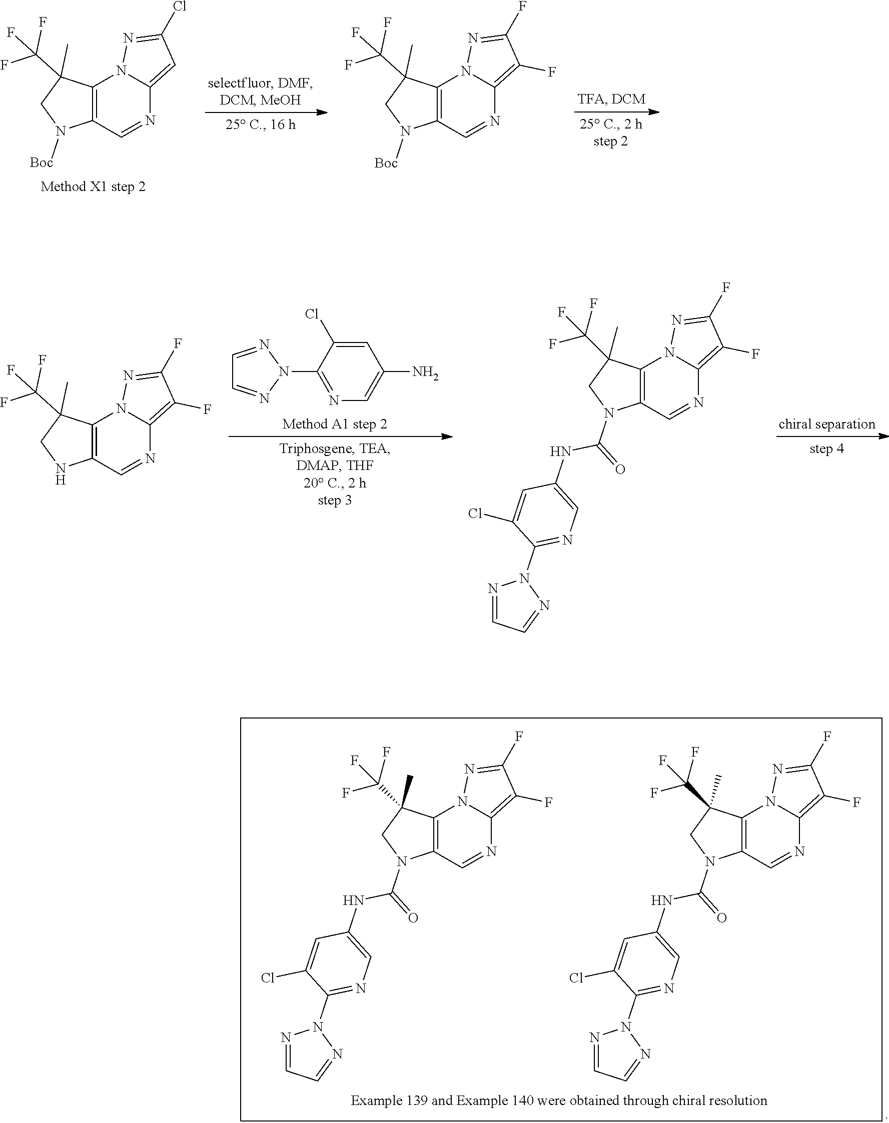

### Examples 139 and 140: Single enantiomers obtained from a racemic mixture containing (S)—N-(5-chloro-6-(2H-1,2,3-triazol-2-yl)pyridin-3-yl)-2,3-difluoro-1-methyl-8-(trifluoromethyl)-7,8-dihydro-6H-pyrazolo[1,5-a]pyrrolo[2,3-e]pyrimidine-6-carboxamide and (R)—N-(5-chloro-6-(2H-1,2,3-triazol-2-yl)pyridin-3-yl)-2,3-difluoro-8-methyl-8-(trifluoromethyl)-7,8-dihydro-6H-pyrazolo[1,5-a]pyrrolo[2,3-e]pyrimidine-6-carboxamide

### Step 1: tert-butyl 2,3-difluoro-8-methyl-8-(trifluoromethyl)-7,8-dihydro-6H-pyrazolo[1,5-a]pyrrolo[2,3-e]pyrimidine-6-carboxylate

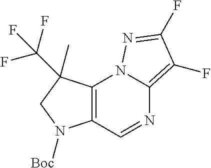 [SMILES: CC(C)(C)OC(=O)N1CC(C)(C(F)(F)F)c2c1cnc1c(F)c(F)nn21]

To a stirred solution of tert-butyl 2-fluoro-8-methyl-8-(trifluoromethyl)-7,8-dihydro-6H-pyrazolo[1,5-a]pyrrolo[2,3-e]pyrimidine-6-carboxylate (Method X1 step 2; 500 mg, 1.4 mmol) in dichloromethane (15 mL) and methanol (15 mL) was added 1-Chloromethyl-4-fluoro-1,4-diazoniabicyclo[2.2.2]octane bis(tetrafluoroborate) (983 mg, 2.8 mmol) in N,N-dimethylformamide (5 mL) at −20° C. The mixture was stirred at 25° C. for 16 h. The reaction mixture was quenched with water (100 mL). The resulting mixture was extracted with ethyl acetate (3×100 mL). The combined organic layers were dried over anhydrous sodium sulfate and concentrated under vacuum. The residue was purified by column chromatography using 90% petroleum ether and 10% ethyl acetate as eluents to afford tert-butyl 2,3-difluoro-8-methyl-8-(trifluoromethyl)-7,8-dihydro-6H-pyrazolo[1,5-a]pyrrolo[2,3-e]pyrimidine-6-carboxylate (110 mg, 20% yield) as an off-white solid. ¹H NMR (400 MHz, Chloroform-d) δ: 9.05 (s, 1H), 4.48 (d, J=12.4 Hz, 1H), 3.80 (d, J=12.4 Hz, 1H), 1.93 (s, 3H), 1.59 (s, 9H). LC-MS: m/z 379 [M+H]⁺.

### Step 2: 2,3-difluoro-8-methyl-8-(trifluoromethyl)-7,8-dihydro-6H-pyrazolo[1,5-a]pyrrolo[2,3-e]pyrimidine

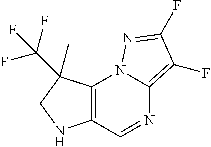 [SMILES: CC1(C(F)(F)F)CNc2cnc3c(F)c(F)nn3c21]

To a stirred solution of tert-butyl 2,3-difluoro-8-methyl-8-(trifluoromethyl)-7,8-dihydro-6H-pyrazolo[1,5-a]pyrrolo[2,3-e]pyrimidine-6-carboxylate (110 mg, 290 μmol) in dichloromethane (4 mL) was added TFA (1 mL) at 0° C. The reaction mixture was stirred at 25° C. for 2 h. The resulting mixture was concentrated under vacuum. The residue was purified by column chromatography using 90% petroleum ether and 10% ethyl acetate as eluents to afford 2,3-difluoro-8-methyl-8-(trifluoromethyl)-7,8-dihydro-6H-pyrazolo[1,5-a]pyrrolo[2,3-e]pyrimidine (80 mg, 98% yield) as a yellow solid. ¹H NMR (400 MHz, Chloroform-d) δ: 8.27 (s, 1H), 4.08 (d, J=11.6 Hz, 1H), 3.56 (d, J=11.6 Hz, 1H), 1.89 (s, 3H). LC-MS: m/z 279 [M+H]⁺.

### Step 3: N-(5-chloro-6-(2H-1,2,3-triazol-2-yl)pyridin-3-yl)-2,3-difluoro-8-methyl-8-(trifluoromethyl)-7,8-dihydro-6H-pyrazolo[1,5-a]pyrrolo[2,3-e]pyrimidine-6-carboxamide

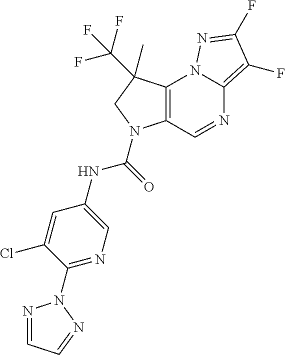 [SMILES: CC1(C(F)(F)F)CN(C(=O)Nc2cnc(-n3nccn3)c(Cl)c2)c2cnc3c(F)c(F)nn3c21]

To a stirred solution of 5-chloro-6-(2H-1,2,3-triazol-2-yl)pyridin-3-amine (Method A1 step 2; 52 mg, 265.3 μmol) in tetrahydrofuran (4 mL) were added triphosgene (32 mg, 108.1 μmol) and TEA (27 mg, 267.3 μmol) at 0° C. The resulting mixture was stirred at 25° C. for 0.5 h and then filtered. The filtrate was added to a solution of 2,3-difluoro-8-methyl-8-(trifluoromethyl)-7,8-dihydro-6H-pyrazolo[1,5-a]pyrrolo[2,3-e]pyrimidine (50 mg, 179.2 μmol) in tetrahydrofuran (2 mL). To this solution was added N,N-dimethylpyridin-4-amine (43 mg, 349.6 μmol) and TEA (181 mg, 1.8 mmol). The mixture was stirred at 40° C. for 2 h. The reaction mixture was quenched with water (10 mL), and extracted with ethyl acetate (3×10 mL). The combined organic layers were washed with brine (20 mL), dried over anhydrous sodium sulfate and concentrated under vacuum. The residue was submitted to Prep-HPLC purification and the collected fractions were lyophilized to give N-(5-chloro-6-(2H-1,2,3-triazol-2-yl)pyridin-3-yl)-2,3-difluoro-8-methyl-8-(trifluoromethyl)-7,8-dihydro-6H-pyrazolo[1,5-a]pyrrolo[2,3-e]pyrimidine-6-carboxamide (42.8 mg, 47% yield) as white solid. ¹H NMR (400 MHz, DMSO-d₆) δ: 9.72 (s, 1H), 9.37 (s, 1H), 8.75 (d, J=2.4 Hz, 1H), 8.50 (d, J=2.4 Hz, 1H), 8.17 (s, 2H), 4.86 (d, J=11.6 Hz, 1H), 4.30 (d, J=11.6 Hz, 1H), 1.95 (s, 3H). LC-MS: m/z 500 [M+H]⁺.

### Step 4: Separation of enantiomers to obtain (S)—N-(5-chloro-6-(2H-1,2,3-triazol-2-yl)pyridin-3-yl)-2,3-difluoro-8-methyl-8-(trifluoromethyl)-7,8-dihydro-6H-pyrazolo[1,5-a]pyrrolo[2,3-e]pyrimidine-6-carboxamide and (R)—N-(5-chloro-6-(2H-1,2,3-triazol-2-yl)pyridin-3-yl)-2,3-difluoro-8-methyl-8-(trifluoromethyl)-7,8-dihydro-6H-pyrazolo[1,5-a]pyrrolo[2,3-e]pyrimidine-6-carboxamide

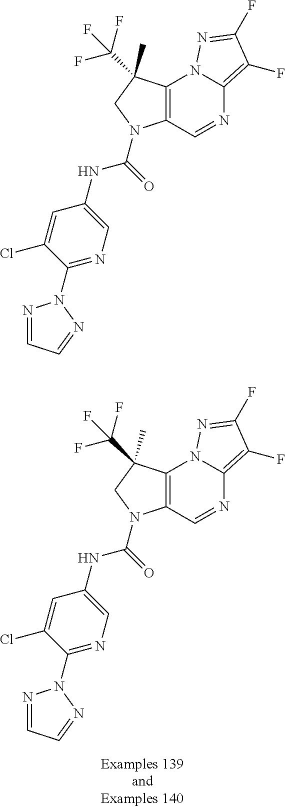 [SMILES: C[C@]1(C(F)(F)F)CN(C(=O)Nc2cnc(-n3nccn3)c(Cl)c2)c2cnc3c(F)c(F)nn3c21 ; C[C@@]1(C(F)(F)F)CN(C(=O)Nc2cnc(-n3nccn3)c(Cl)c2)c2cnc3c(F)c(F)nn3c21]

N-(5-chloro-6-(2H-1,2,3-triazol-2-yl)pyridin-3-yl)-2,3-difluoro-8-methyl-8-(trifluoromethyl)-7,8-dihydro-6H-pyrazolo[1,5-a]pyrrolo[2,3-e]pyrimidine-6-carboxamide (40 mg, 80 μmol) was submitted to chiral HPLC purification: Column: CHIRAL ART Cellulose-SC, 2×25 cm, 5 um; Mobile Phase A: Hex:DCM=3:1 (0.5% 2M NH₃-MeOH)—HPLC, Mobile Phase B: EtOH—HPLC; Flow rate: 20 mL/min; Gradient: 15 B to 15 B in 11 min; 254/220 nm; RT1: 8.430; RT2: 9.412; Injection Volume: 0.6 ml; Number Of Runs: 6; The first eluting isomer was concentrated and lyophilized to afford Example 140 as a white solid (7.1 mg, 17% yield). The second eluting isomer was concentrated and lyophilized to afford Example 139 as a white solid (5.9 mg, 14% yield). Examples 139 and 140 are enantiomers, but their absolute stereochemistry is not yet known.

Example 139: ¹H NMR (300 MHz, DMSO-d₆) δ: 9.72 (s, 1H), 9.37 (s, 1H), 8.74 (s, 1H), 8.50 (s, 1H), 8.17 (s, 2H), 4.86 (d, J=11.4 Hz, 1H), 4.30 (d, J=11.4 Hz, 1H), 1.95 (s, 3H) LC-MS: m/z 500 [M+H]⁺.

Example 140: ¹H NMR (300 MHz, DMSO-d₆) δ: 9.72 (s, 1H), 9.37 (s, 1H), 8.75 (d, J=2.4 Hz, 1H), 8.50 (d, J=2.4 Hz, 1H), 8.17 (s, 2H), 4.86 (d, J=11.4 Hz, 1H), 4.30 (d, J=11.4 Hz, 1H), 1.95 (s, 3H) LC-MS: m/z 500 [M+H]⁺.

### Method W4

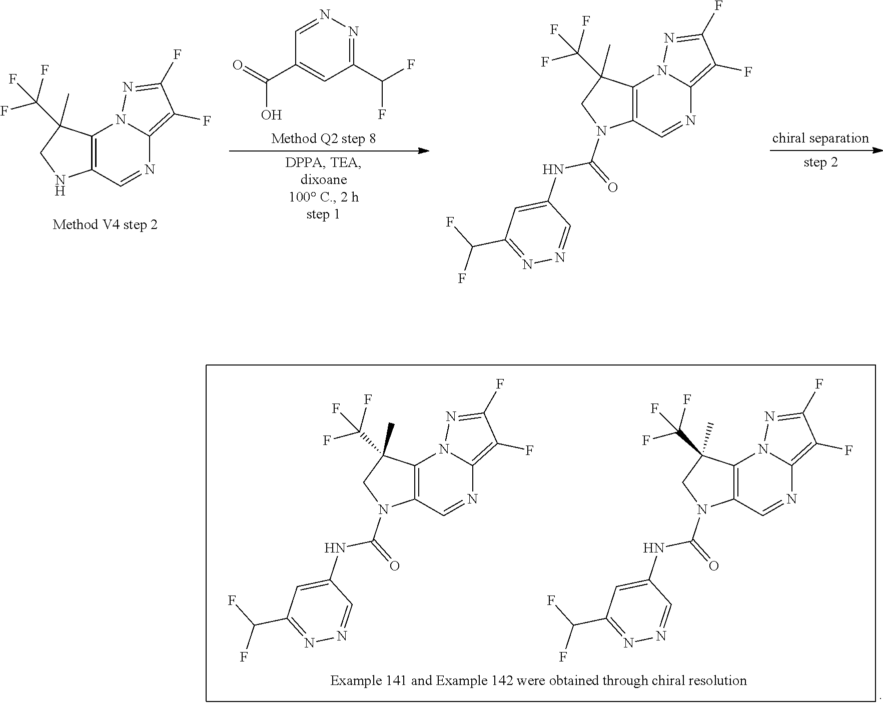 [SMILES: CC1(C(F)(F)F)CNc2cnc3c(F)c(F)nn3c21 ; O=C(O)c1cnnc(C(F)F)c1 ; CC1(C(F)(F)F)CN(C(=O)Nc2cnnc(C(F)F)c2)c2cnc3c(F)c(F)nn3c21 ; C[C@]1(C(F)(F)F)CN(C(=O)Nc2cnnc(C(F)F)c2)c2cnc3c(F)c(F)nn3c21 ; C[C@@]1(C(F)(F)F)CN(C(=O)Nc2cnnc(C(F)F)c2)c2cnc3c(F)c(F)nn3c21]

### Examples 141 and 142: Single enantiomers obtained from a racemic mixture containing (S)—N-(6-(difluoromethyl)pyridazin-4-yl)-2,3-difluoro-8-methyl-8-(trifluoromethyl)-7,8-dihydro-6H-pyrazolo[1,5-a]pyrrolo[2,3-e]pyrimidine-6-carboxamide and (R)—N-(6-(difluoromethyl)pyridazin-4-yl)-2,3-difluoro-8-methyl-8-(trifluoromethyl)-7,8-dihydro-6H-pyrazolo[1,5-a]pyrrolo[2,3-e]pyrimidine-6-carboxamide

### Step 1: N-(6-(difluoromethyl)pyridazin-4-yl)-2,3-difluoro-8-methyl-8-(trifluoromethyl)-7,8-dihydro-6H-pyrazolo[1,5-a]pyrrolo[2,3-e]pyrimidine-6-carboxamide

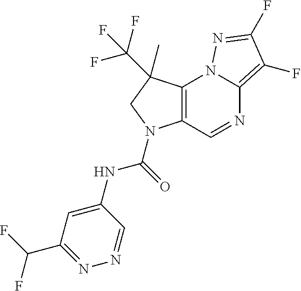 [SMILES: CC1(C(F)(F)F)CN(C(=O)Nc2cnnc(C(F)F)c2)c2cnc3c(F)c(F)nn3c21]

To a stirred solution of 2,3-difluoro-8-methyl-8-(trifluoromethyl)-7,8-dihydro-6H-pyrazolo[1,5-a]pyrrolo[2,3-e]pyrimidine (Method V4 step 2; 64 mg, 230.2 μmol) and 6-(difluoromethyl)pyridazine-4-carboxylic acid (Method Q2 step 8; 40 mg, 229.9 μmol) in dioxane (5 mL) were added DPPA (75 mg, 276.2 μmol) and TEA (116 mg, 1.1 mmol). The mixture was stirred at 100° C. for 2 h. After cooled to 25° C., the mixture was concentrated under vacuum. The residue was submitted to Prep-HPLC purification and the collected fractions were lyophilized to give N-(6-(difluoromethyl)pyridazin-4-yl)-2,3-difluoro-8-methyl-8-(trifluoromethyl)-7,8-dihydro-6H-pyrazolo[1,5-a]pyrrolo[2,3-e]pyrimidine-6-carboxamide (62.3 mg, 60% yield) as a light-yellow solid. ¹H NMR (400 MHz, DMSO-d₆) δ: 9.93 (s, 1H), 9.50 (d, J=2.4 Hz, 1H), 9.35 (s, 1H), 8.21 (d, J=2.8 Hz, 1H), 7.24 (t, J=55.4 Hz, 1H), 4.87 (d, J=11.6 Hz, 1H), 4.30 (d, J=11.6 Hz, 1H), 1.94 (s, 3H). LC-MS: m/z 450 [M+H]⁺.

### Step 2: Separation of enantiomers to obtain (S)—N-(6-(difluoromethyl)pyridazin-4-yl)-2,3-difluoro-8-methyl-8-(trifluoromethyl)-7,8-dihydro-6H-pyrazolo[1,5-a]pyrrolo[2,3-e]pyrimidine-6-carboxamide and (R)—N-(6-(difluoromethyl)pyridazin-4-yl)-2,3-difluoro-8-methyl-8-(trifluoromethyl)-7,8-dihydro-6H-pyrazolo[1,5-a]pyrrolo[2,3-e]pyrimidine-6-carboxamide

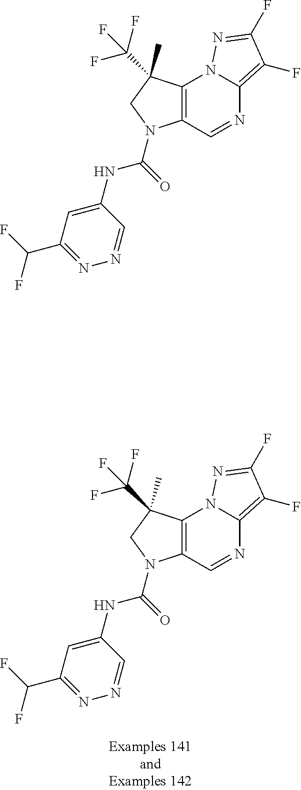 [SMILES: C[C@]1(C(F)(F)F)CN(C(=O)Nc2cnnc(C(F)F)c2)c2cnc3c(F)c(F)nn3c21 ; C[C@@]1(C(F)(F)F)CN(C(=O)Nc2cnnc(C(F)F)c2)c2cnc3c(F)c(F)nn3c21]

N-(6-(difluoromethyl)pyridazin-4-yl)-2,3-difluoro-8-methyl-8-(trifluoromethyl)-7,8-dihydro-6H-pyrazolo[1,5-a]pyrrolo[2,3-e]pyrimidine-6-carboxamide (60 mg, 133 μmol) was submitted to chiral HPLC purification: Column: CHIRAL ART Cellulose-SB, 2×25 cm, 5 um; Mobile Phase A: Hex (0.5% 2M NH₃-MeOH)-HPLC, Mobile Phase B: EtOH-HPLC; Flow rate: 20 mL/L/min; Gradient: 20 B to 20 B in 16 min; 220/254 nm; RT1: 6.342; RT2: 11.264; Injection Volume: 4 ml; Number Of Runs: 3; The first eluting isomer was concentrated and lyophilized to afford Example 141 (9.4 mg, 15% yield) as a white solid. The second eluting isomer was concentrated and lyophilized to afford Example 142 (7.6 mg, 12% yield) as a white solid. Examples 141 and 142 are enantiomers, but their absolute stereochemistry is not yet known.

Example 141: ¹H NMR (300 MHz, DMSO-d₆) δ: 9.93 (s, 1H), 9.50 (d, J=2.4 Hz, 1H), 9.36 (s, 1H), 8.21 (d, J=2.7 Hz, 1H), 7.24 (t, J=54.3 Hz, 1H), 4.87 (d, J=11.7 Hz, 1H), 4.30 (d, J=11.7 Hz, 1H), 1.93 (s, 3H). LC-MS: m/z 450 [M+H]⁺.

Example 142: ¹H NMR (300 MHz, DMSO-d₆) δ: 9.93 (s, 1H), 9.50 (d, J=2.1 Hz, 1H), 9.36 (s, 1H), 8.21 (d, J=2.4 Hz, 1H), 7.24 (t, J=54.3 Hz, 1H), 4.87 (d, J=8.7 Hz, 1H), 4.31 (d, J=8.7 Hz, 1H), 1.94 (s, 3H). LC-MS: m/z 450 [M+H]⁺.

### Method X4

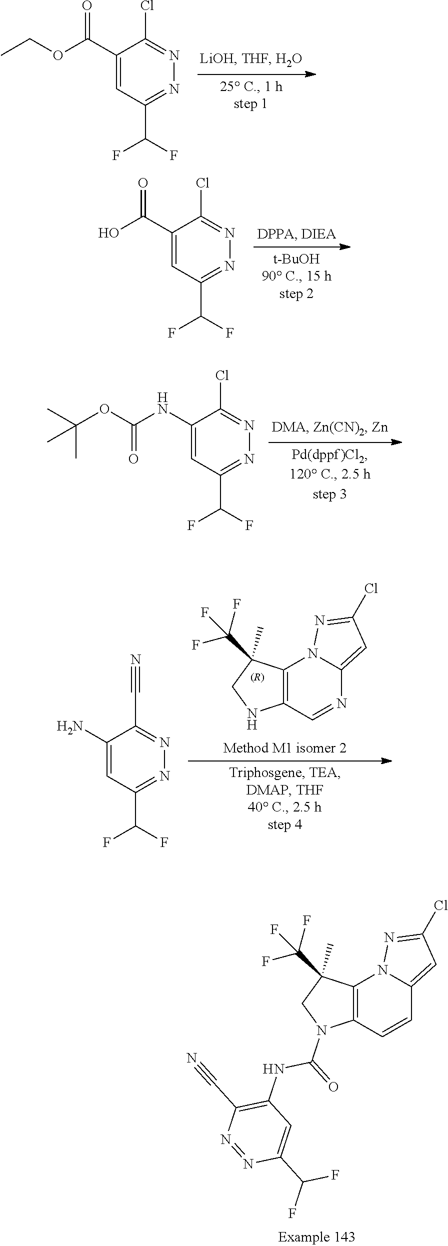

### Example 143: (R)-2-chloro-N-(3-cyano-6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide

### Step 1: 3-chloro-6-(difluoromethyl)pyridazine-4-carboxylic acid

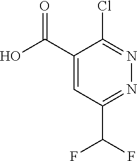 [SMILES: O=C(O)c1cc(C(F)F)nnc1Cl]

To a stirred solution of ethyl 3-chloro-6-(difluoromethyl)pyridazine-4-carboxylate (800 mg, 3.4 mmol) in tetrahydrofuran (8 mL) and water (2 mL) was added lithium hydroxide (242.9 mg, 10.1 mmol) at 0° C. The reaction mixture was stirred at 25° C. for 1 h. The mixture was concentrated under reduced pressure. Then the resulting solution was diluted with water (30 mL). The pH was adjusted to 3 with HCl (1 M). The resulting solution was extracted with ethyl acetate (3×30 mL). The combined organic extracts were dried over anhydrous sodium sulfate and concentrated under reduced pressure. The obtained solid was washed with n-pentane (20 mL) to afford 3-chloro-6-(difluoromethyl)pyridazine-4-carboxylic acid (590 mg, 83% yield) as an off-white solid. ¹H NMR (400 MHz, DMSO-d₆) δ: 8.32 (s, 1H), 7.35 (t, J=53.6 Hz, 1H). LC-MS: m/z 209 [M+H]⁺.

### Step 2: tert-butyl (3-chloro-6-(difluoromethyl)pyridazin-4-yl)carbamate

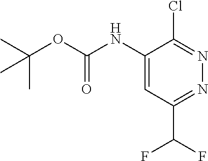 [SMILES: CC(C)(C)OC(=O)Nc1cc(C(F)F)nnc1Cl]

To a stirred solution of 3-chloro-6-(difluoromethyl)pyridazine-4-carboxylic acid (200 mg, 959.0 μmol) in 2-methylpropan-2-ol (10 mL) was added DPPA (527.8 mg, 1.9 mmol) and DIEA (247.9 mg, 1.9 mmol). The resulting mixture was stirred at 90° C. for 15 h under nitrogen atmosphere. The mixture was allowed to cool down to 25° C. and concentrated under vacuum. The residue was diluted with water (50 mL) and extracted with ethyl acetate (3×50 mL). The combined organic layers were dried over anhydrous sodium sulfate and concentrated under vacuum. The residue was purified by column chromatography on silica gel using 90% petroleum ether and 10% ethyl acetate as eluents to afford tert-butyl (3-chloro-6-(difluoromethyl)pyridazin-4-yl)carbamate (170 mg, 63.4% yield) as a white solid. LC-MS: m/z 280 [M+H]⁺.

### Step 3: 4-amino-6-(difluoromethyl)pyridazine-3-carbonitrile

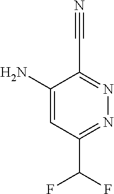 [SMILES: N#Cc1nnc(C(F)F)cc1N]

To a stirred solution of tert-butyl (3-chloro-6-(difluoromethyl)pyridazin-4-yl)carbamate (120 mg, 429.1 μmol) in N,N-dimethylacetamide (6 mL) under nitrogen atmosphere was added zinc cyanide (50.4 mg, 429.1 μmol), zinc (2.8 mg, 42.9 μmol) and Pd(dppf)Cl₂ (31.4 mg, 42.9 μmol). The resulting mixture was stirred at 120° C. for 2.5 h under nitrogen atmosphere. The mixture was allowed to cool down to 25° C. The reaction mixture was quenched with water (50 mL) and extracted with ethyl acetate (3×50 mL). The combined organic layers were washed with brine (3×50 mL), dried over anhydrous sodium sulfate and concentrated under vacuum. The residue was purified by column chromatography on silica gel using 70% petroleum ether and 30% ethyl acetate as eluents to afford 4-amino-6-(difluoromethyl)pyridazine-3-carbonitrile (54 mg, 70% yield) as a white solid. LC-MS: m/z 171 [M+H]⁺.

### Step 4: (R)-2-chloro-N-(3-cyano-6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide

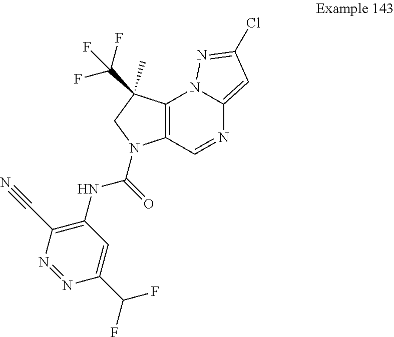 [SMILES: C[C@@]1(C(F)(F)F)CN(C(=O)Nc2cc(C(F)F)nnc2C#N)c2cnc3cc(Cl)nn3c21]

To a stirred solution of 4-amino-6-(difluoromethyl)pyridazine-3-carbonitrile (25 mg, 146.9 μmol) in tetrahydrofuran (8 mL) was added triphosgene (34.9 mg, 117.6 μmol) and TEA (22.3 mg, 220.4 μmol). The resulting mixture was stirred at 40° C. for 0.5 h and then filtered. The resulting filtrate was added to a solution of Method M1 isomer 2 (40.7 mg, 146.9 μmol) in tetrahydrofuran (1.5 mL). To this solution was then added TEA (148.7 mg, 1.8 mmol) and N,N-dimethylpyridin-4-amine (35.9 mg, 293.9 μmol). The mixture was stirred at 40° C. for 2 h. The solvent was concentrated under vacuum. The residue was purified by Prep-TLC using 97% dichloromethane and 3% methanol as eluents to afford 30 mg of the crude product which was purified by Prep-HPLC purification and the collected fractions were lyophilized to give (R)-2-chloro-N-(3-cyano-6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide (2 mg, 2.80% yield) as a light-yellow solid. The enantiomer of Example 143 can be prepared analogously using Method M1 isomer 1.

Example 143: ¹H NMR (400 MHz, DMSO-d₆) δ: 10.27 (br, 1H), 9.32 (s, 1H), 8.33 (s, 1H), 7.38 (t, J=54.0 Hz, 1H), 7.10 (s, 1H), 4.87 (d, J=10.8 Hz, 1H), 4.32 (d, J=10.8 Hz, 1H), 1.99 (s, 3H). LC-MS: m/z 473 [M+H]⁺.

### Method Y4

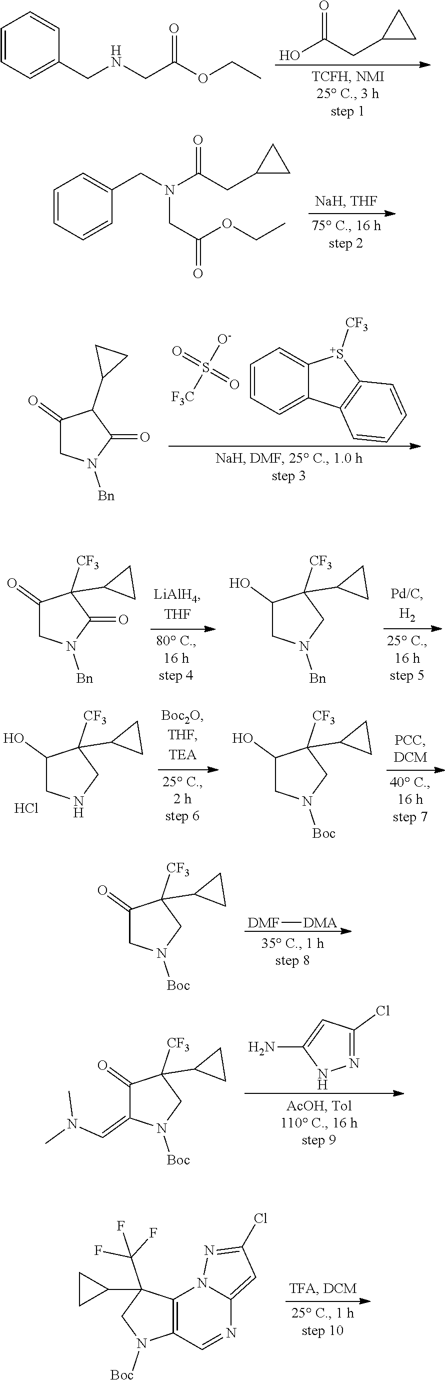

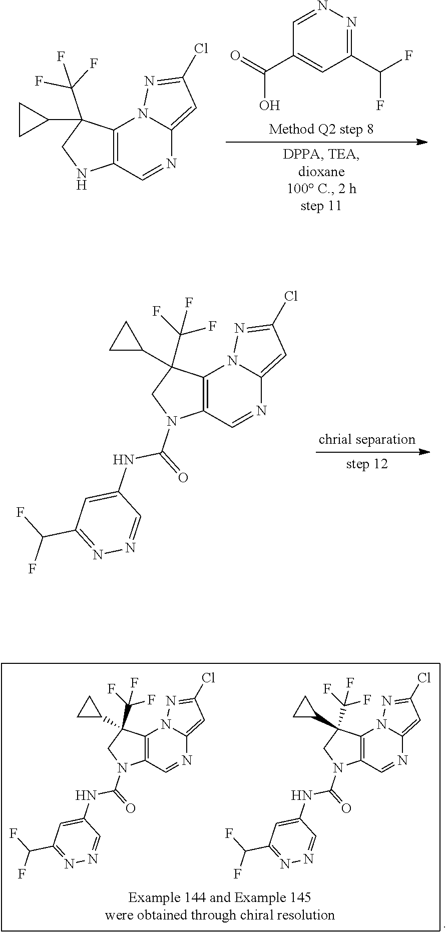 [SMILES: FC(F)(F)C1(C2CC2)CNc2cnc3cc(Cl)nn3c21 ; O=C(O)c1cnnc(C(F)F)c1 ; O=C(Nc1cnnc(C(F)F)c1)N1CC(C2CC2)(C(F)(F)F)c2c1cnc1cc(Cl)nn21 ; O=C(Nc1cnnc(C(F)F)c1)N1C[C@@](C2CC2)(C(F)(F)F)c2c1cnc1cc(Cl)nn21 ; O=C(Nc1cnnc(C(F)F)c1)N1C[C@](C2CC2)(C(F)(F)F)c2c1cnc1cc(Cl)nn21]

### Examples 144 and 145: Single enantiomers obtained from a racemic mixture containing (R)-2-chloro-8-cyclopropyl-N-(6-(difluoromethyl)pyridazin-4-yl)-8-(trifluoromethyl)-7,8-dihydro-6H-pyrazolo[1,5-a]pyrrolo[2,3-e]pyrimidine-6-carboxamide

### (S)-2-chloro-8-cyclopropyl-N-(6-(difluoromethyl)pyridazin-4-yl)-8-(trifluoromethyl)-7,8-dihydro-6H-pyrazolo[1,5-a]pyrrolo[2,3-e]pyrimidine-6-carboxamide

### Step 1: ethyl N-benzyl-N-(2-cyclopropylacetyl)glycinate

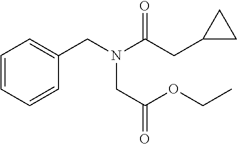 [SMILES: CCOC(=O)CN(Cc1ccccc1)C(=O)CC1CC1]

To a stirred mixture of ethyl benzylglycinate (15.0 g, 77.6 mmol) in acetonitrile (150 mL) was added 2-cyclopropylacetic acid (9.3 g, 93.2 mmol), 1-methylimidazole (19.1 g, 232.9 mmol) and N,N,N,N-tetramethylchloroformamidinium hexafluorophosphate (65.5 g, 232.9 mmol). The reaction mixture was stirred at 25° C. for 3 h. The mixture was concentrated under vacuum. The residue was purified by column chromatography on silica gel using 80% petroleum ether and 20% ethyl acetate as eluents to afford ethyl N-benzyl-N-(2-cyclopropylacetyl)glycinate (19.6 g, 91% yield) as a yellow oil. ¹H NMR (400 MHz, DMSO-d₆) δ: 7.23-7.38 (m, 5H), 4.62 (s, 1H), 4.51 (s, 1H), 4.10 (s, 1H), 4.04-4.08 (m, 2H), 4.00 (s, 1H), 2.31 (d, J=6.4 Hz, 1H), 2.24 (d, J=6.8 Hz, 1H), 1.13-1.19 (m, 3H), 0.90-1.04 (m, 1H), 0.40-0.47 (m, 2H), 0.03-0.11 (m, 2H). LC-MS: m/z 276 [M+H]⁺.

### Step 2: 1-benzyl-3-cyclopropylpyrrolidine-2,4-dione

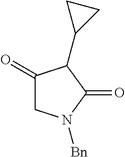 [SMILES: O=C1CN(Cc2ccccc2)C(=O)C1C1CC1]

To a stirred solution of tetrahydrofuran (100 mL) was added NaH (4.0 g, 98.4 mmol, 60% in mineral oil) in batches at 0° C. The reaction mixture was stirred at 75° C. and the solution of ethyl N-benzyl-N-(2-cyclopropylacetyl)glycinate (22.5 g, 81.72 mmol) in tetrahydrofuran (100 mL) was added dropwise. The reaction mixture was stirred at 75° C. for 16 h. The reaction mixture was quenched with water (100 mL). The pH was adjusted to 3-4 with HCl (1 M). The resulting solution was extracted with ethyl acetate (3×100 mL). The combined organic layers were dried over anhydrous sodium sulfate and concentrated under vacuum. The residue was purified by column chromatography on silica gel using 98% dichloromethane and 2% methanol as eluents to afford 1-benzyl-3-cyclopropylpyrrolidine-2,4-dione (12.9 g, 68.8% yield) as a light yellow solid. ¹H NMR (400 MHz, DMSO-d₆) δ: 10.55 (s, 1H), 7.15-7.35 (m, 5H), 4.43 (s, 2H), 3.60 (s, 2H), 1.41-1.48 (m, 1H), 0.89-0.92 (m, 2H), 0.57-0.62 (m, 2H). LC-MS: m/z 230 [M+H]⁺.

### Step 3: 1-benzyl-3-cyclopropyl-3-(trifluoromethyl)pyrrolidine-2,4-dione

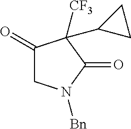 [SMILES: O=C1CN(Cc2ccccc2)C(=O)C1(C1CC1)C(F)(F)F]

To a stirred solution of 1-benzyl-3-cyclopropylpyrrolidine-2,4-dione (7.0 g, 30.5 mmol) in N,N-dimethylacetamide (100 mL) was added NaH (1.3 g, 33.6 mmol, 60% in mineral oil) in batches at 0° C. The reaction mixture was stirred at 25° C. for 0.5 h. 5-(trifluoromethyl)-5H-dibenzo[b,d]thiophen-5-ium trifluoromethanesulfonate (12.1 g, 30.5 mmol) was added at −55° C. The reaction mixture was stirred at −55° C. for 1 h and stirred at 25° C. for another 1 h. The reaction mixture was quenched with water (400 mL). The resulting solution was extracted with ethyl acetate (3×400 mL). The combined organic layers were washed with brine (3×800 mL), dried over anhydrous sodium sulfate and concentrated under vacuum. The residue was purified by column chromatography on silica gel using 90% petroleum ether and 10% ethyl acetate as eluents to afford 1-benzyl-3-cyclopropyl-3-(trifluoromethyl)pyrrolidine-2,4-dione (4.9 g, 55.1% yield) as a yellow solid. ¹H NMR (400 MHz, DMSO-d₆) δ: 7.23-7.41 (m, 5H), 4.63 (s, 2H), 4.04-4.15 (m, 2H), 1.35-1.41 (m, 1H), 0.67-0.73 (m, 1H), 0.57-0.65 (m, 2H), 0.31-0.39 (m, 1H). LC-MS: m/z 298 [M+H]⁺.

### Step 4: 1-benzyl-4-cyclopropyl-4-(trifluoromethyl)pyrrolidin-3-ol

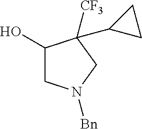 [SMILES: OC1CN(Cc2ccccc2)CC1(C1CC1)C(F)(F)F]

To a stirred solution of 1-benzyl-3-cyclopropyl-3-(trifluoromethyl)pyrrolidine-2,4-dione (4.3 g, 14.5 mmol) in tetrahydrofuran (60 mL) was added LiAlH₄ (2.2 g, 57.9 mmol) at 0° C. The reaction mixture was stirred at 80° C. for 16 h. The reaction mixture was cooled to 0° C. While stirring, H₂O (2.2 g) and an aqueous solution of NaOH (10%, 2.2 g) were added, followed by the addition of H₂O (2.2 g). The resulting mixture was filtered and concentrated under vacuum. The residue was purified by column chromatography on silica gel using 98% dichloromethane and 2% methanol as eluents to afford 1-benzyl-4-cyclopropyl-4-(trifluoromethyl)pyrrolidin-3-ol (2.2 g, 7.71 mmol, 74.79% yield) as a light yellow solid. LC-MS: m/z 286 [M+H]⁺.

### Step 5: 4-cyclopropyl-4-(trifluoromethyl)pyrrolidin-3-ol hydrochloride

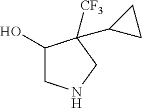 [SMILES: OC1CNCC1(C1CC1)C(F)(F)F]

To a stirred solution of 1-benzyl-4-cyclopropyl-4-(trifluoromethyl)pyrrolidin-3-ol (2.2 g, 7.7 mmol) in ethanol (60 mL) was added Pd/C (1.0 g, 10%) and HCl (1 M, 7.7 mL). The reaction mixture was stirred at 25° C. for 16 h under hydrogen. Then HCl (1 M, 7.7 mL) was added at 25° C. and the reaction mixture was stirred at 25° C. for another 0.5 h. The solid was filtered out. The filtrate was concentrated under vacuum to afford 4-cyclopropyl-4-(trifluoromethyl)pyrrolidin-3-ol hydrochloride (1.5 g, crude) as a brown solid. LC-MS: m/z 196 [M+H]⁺.

### Step 6: tert-butyl 3-cyclopropyl-4-hydroxy-3-(trifluoromethyl)pyrrolidine-1-carboxylate

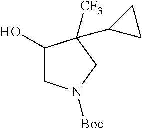 [SMILES: CC(C)(C)OC(=O)N1CC(O)C(C2CC2)(C(F)(F)F)C1]

To a stirred solution of 4-cyclopropyl-4-(trifluoromethyl)pyrrolidin-3-ol hydrochloride (1.5 g, 7.7 mmol) in tetrahydrofuran (20 mL) was added (Boc)₂O (2.5 g, 11.6 mmol) and TEA (3.9 g, 38.5 mmol). The reaction mixture was stirred at 25° C. for 2 h. The mixture was concentrated under vacuum. The residue was purified by column chromatography on silica gel using 80% petroleum ether and 20% ethyl acetate as eluents to afford tert-butyl 3-cyclopropyl-4-hydroxy-3-(trifluoromethyl)pyrrolidine-1-carboxylate (2.1 g, 94.1% yield) as a light yellow oil. LC-MS: m/z 296 [M+H]⁺.

### Step 7: tert-butyl 3-cyclopropyl-4-oxo-3-(trifluoromethyl)pyrrolidine-1-carboxylate

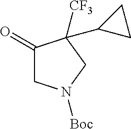 [SMILES: CC(C)(C)OC(=O)N1CC(=O)C(C2CC2)(C(F)(F)F)C1]

To a stirred mixture of tert-butyl 3-cyclopropyl-4-hydroxy-3-(trifluoromethyl)pyrrolidine-1-carboxylate (2.2 g, 7.3 mmol) in dichloromethane (50 mL) was added PCC (7.8 g, 36.3 mmol) and silica gel (16.0 g) at 25° C. The reaction mixture was stirred at 40° C. for 16 h. The solid was filtered out. The filtrate was concentrated under vacuum. The residue was purified by column chromatography using 90% petroleum ether and 10% ethyl acetate as eluents to afford tert-butyl 3-cyclopropyl-4-oxo-3-(trifluoromethyl)pyrrolidine-1-carboxylate (1.4 g, 63.1% yield) as a yellow oil. ¹H NMR (400 MHz, DMSO-d₆) δ: 3.89-4.08 (m, 2H), 3.65-3.75 (m, 1H), 3.46 (d, J=12.4 Hz, 1H), 1.43 (s, 9H), 1.18-1.26 (m, 1H), 0.54-0.64 (m, 2H), 0.34-0.44 (m, 2H). LC-MS: m/z 294 [M+H]⁺.

### Step 8: tert-butyl (E)-4-cyclopropyl-2-((dimethylamino)methylene)-3-oxo-4-(trifluoromethyl)pyrrolidine-1-carboxylate

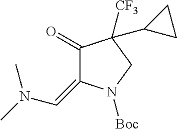 [SMILES: CN(C)/C=C1\C(=O)C(C2CC2)(C(F)(F)F)CN1C(=O)OC(C)(C)C]

A mixture of tert-butyl 3-cyclopropyl-4-oxo-3-(trifluoromethyl)pyrrolidine-1-carboxylate (1.3 g, 4.5 mmol) in DMF-DMA (15 mL) was stirred at 35° C. for 1 h. The reaction mixture was concentrated under vacuum to afford tert-butyl (E)-4-cyclopropyl-2-((dimethylamino)methylene)-3-oxo-4-(trifluoromethyl)pyrrolidine-1-carboxylate (1.5 g, crude) as a yellow solid. LC-MS: m/z 349 [M+H]⁺.

### Step 9: tert-butyl 2-chloro-8-cyclopropyl-8-(trifluoromethyl)-7,8-dihydro-6H-pyrazolo[1,5-a]pyrrolo[2,3-e]pyrimidine-6-carboxylate

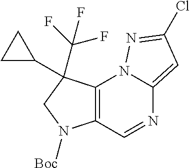 [SMILES: CC(C)(C)OC(=O)N1CC(C2CC2)(C(F)(F)F)c2c1cnc1cc(Cl)nn21]

To a stirred solution of tert-butyl (E)-4-cyclopropyl-2-((dimethylamino)methylene)-3-oxo-4-(trifluoromethyl)pyrrolidine-1-carboxylate (1.5 g, 4.2 mmol) in toluene (20 mL) was added acetic acid (2 mL) and 3-chloro-1H-pyrazol-5-amine (489 mg, 4.2 mmol). The reaction mixture was stirred at 110° C. for 16 h. The mixture was concentrated under vacuum. The residue was diluted with saturated aqueous NaHCO₃ solution (40 mL). The resulting solution was extracted with ethyl acetate (3×40 mL). The combined organic layers were dried over anhydrous sodium sulfate and concentrated under vacuum. The residue was purified by column chromatography using 90% petroleum ether and 10% ethyl acetate as eluents to afford tert-butyl 2-chloro-8-cyclopropyl-8-(trifluoromethyl)-7,8-dihydro-6H-pyrazolo[1,5-a]pyrrolo[2,3-e]pyrimidine-6-carboxylate (567 mg, 33.8% yield) as a yellow oil. LC-MS: m/z 403 [M+H]⁺.

### Step 10: 2-chloro-8-cyclopropyl-8-(trifluoromethyl)-7,8-dihydro-6H-pyrazolo[1,5-a]pyrrolo[2,3-e]pyrimidine

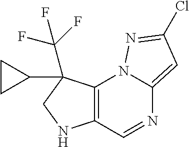 [SMILES: FC(F)(F)C1(C2CC2)CNc2cnc3cc(Cl)nn3c21]

To a solution of tert-butyl 2-chloro-8-cyclopropyl-8-(trifluoromethyl)-7,8-dihydro-6H-pyrazolo[1,5-a]pyrrolo[2,3-e]pyrimidine-6-carboxylate (550 mg, 1.4 mmol) in dichloromethane (9 mL) was added TFA (3 mL) at 25° C. The reaction mixture was stirred at 25° C. for 1 h and concentrated under vacuum. The residue was diluted with saturated aqueous NaHCO₃ solution (30 mL). The resulting mixture was extracted with ethyl acetate (3×30 mL). The combined organic layers were dried over anhydrous sodium sulfate and concentrated under vacuum. The residue was purified by column chromatography using 99% dichloromethane and 1% methanol as eluents to afford 2-chloro-8-cyclopropyl-8-(trifluoromethyl)-7,8-dihydro-6H-pyrazolo[1,5-a]pyrrolo[2,3-e]pyrimidine (197 mg, 72.6% yield) as a light-yellow solid. ¹H NMR (400 MHz, DMSO-d₆) δ: 8.39 (s, 1H), 6.89 (s, 1H), 5.89 (s, 1H), 3.56 (d, J=12.0 Hz, 1H), 3.18 (d, J=12.0 Hz, 1H), 2.00-2.07 (m, 1H), 0.67-0.74 (m, 1H), 0.42-0.51 (m, 3H). LC-MS: m/z 303 [M+H]⁺.

### Step 11: 2-chloro-8-cyclopropyl-N-(6-(difluoromethyl)pyridazin-4-yl)-8-(trifluoromethyl)-7,8-dihydro-6H-pyrazolo[1,5-a]pyrrolo[2,3-e]pyrimidine-6-carboxamide

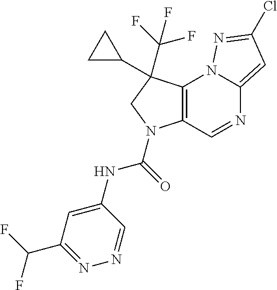 [SMILES: O=C(Nc1cnnc(C(F)F)c1)N1CC(C2CC2)(C(F)(F)F)c2c1cnc1cc(Cl)nn21]

To a stirred solution of 2-chloro-8-cyclopropyl-8-(trifluoromethyl)-7,8-dihydro-6H-pyrazolo[1,5-a]pyrrolo[2,3-e]pyrimidine (70 mg, 231.3 μmol) in dioxane (10 mL) was added 6-(difluoromethyl)pyridazine-4-carboxylic acid (Method Q2 step 8; 40 mg, 231.3 μmol), DPPA (71 mg, 277.5 μmol) and TEA (116 mg, 1.2 mmol). The resulting mixture was stirred at 100° C. for 2 h. The mixture was allowed to cool down to 25° C. and concentrated under vacuum. The residue was purified by Prep-TLC using 4% methanol and 96% dichloromethane as eluents to afford 80 mg of the crude product which was submitted to Prep-HPLC purification and the collected fractions were lyophilized to afford 2-chloro-8-cyclopropyl-N-(6-(difluoromethyl)pyridazin-4-yl)-8-(trifluoromethyl)-7,8-dihydro-6H-pyrazolo[1,5-a]pyrrolo[2,3-e]pyrimidine-6-carboxamide (50 mg, 45.9% yield) as a white solid. ¹H NMR (400 MHz, DMSO-d₆) δ: 9.79 (s, 1H), 9.49 (d, J=2.4 Hz, 1H), 9.37 (s, 1H), 8.19 (d, J=2.4 Hz, 1H), 7.25 (t, J=53.8 Hz, 1H), 7.12 (s, 1H), 4.43 (d, J=11.6 Hz, 1H), 3.81 (d, J=12.0 Hz, 1H), 2.26-2.29 (m, 1H), 0.83-0.92 (m, 1H), 0.59-0.67 (m, 1H), 0.48-0.56 (m, 2H). LC-MS: m/z 474 [M+H]⁺.

### Step 12: Separation of enantiomers to obtain (R)-2-chloro-8-cyclopropyl-N-(6-(difluoromethyl)pyridazin-4-yl)-8-(trifluoromethyl)-7,8-dihydro-6H-pyrazolo[1,5-a]pyrrolo[2,3-e]pyrimidine-6-carboxamide and (S)-2-chloro-8-cyclopropyl-N-(6-(difluoromethyl)pyridazin-4-yl)-8-(trifluoromethyl)-7,8-dihydro-6H-pyrazolo[1,5-a]pyrrolo[2,3-e]pyrimidine-6-carboxamide

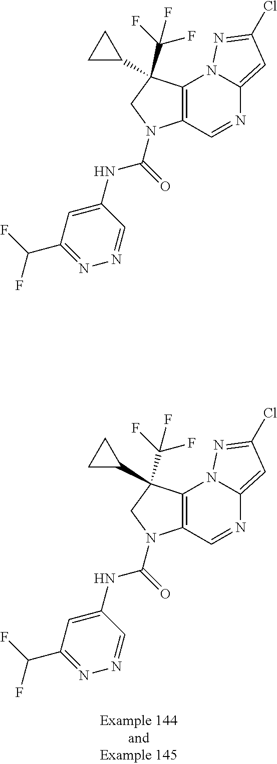 [SMILES: O=C(Nc1cnnc(C(F)F)c1)N1C[C@@](C2CC2)(C(F)(F)F)c2c1cnc1cc(Cl)nn21 ; O=C(Nc1cnnc(C(F)F)c1)N1C[C@](C2CC2)(C(F)(F)F)c2c1cnc1cc(Cl)nn21]

2-chloro-8-cyclopropyl-N-(6-(difluoromethyl)pyridazin-4-yl)-8-(trifluoromethyl)-7,8-dihydro-6H-pyrazolo[1,5-a]pyrrolo[2,3-e]pyrimidine-6-carboxamide (50 mg, 105.5 μmol) was submitted to chiral-HPLC: Column: CHIRALPAK IA, 2×25 cm, 5 um; Mobile Phase A: Hex (0.5% 2M NH₃-MeOH)-HPLC, Mobile Phase B: EtOH—HPLC; Flow rate: 20 mL/min; Gradient: 20 B to 20 B in 12 min; 254/220 nm; RT1: 7.818; RT2: 9.92; Injection Volume: 0.8 ml; Number Of Runs: 5. The first eluting isomer was concentrated and lyophilized to afford Example 144 (18.8 mg, 37.6% yield) as a white solid. The second eluting isomer was concentrated and lyophilized to afford Example 145 (16.7 mg, 33.4% yield) as a white solid. Examples 144 and 145 are enantiomers, but their absolute stereochemistry is not yet known.

Example 144: ¹H NMR (400 MHz, DMSO-d₆) δ: 9.79 (s, 1H), 9.50 (d, J=2.4 Hz, 1H), 9.37 (s, 1H), 8.19 (d, J=2.8 Hz, 1H), 7.25 (t, J=53.8 Hz, 1H), 7.12 (s, 1H), 4.43 (d, J=12.0 Hz, 1H), 3.81 (d, J=12.4 Hz, 1H), 2.23-2.29 (m, 1H), 0.83-0.92 (m, 1H), 0.60-0.67 (m, 1H), 0.48-0.56 (m, 2H). LC-MS: m/z 474 [M+H]⁺.

Example 145: ¹H NMR (400 MHz, DMSO-d₆) δ: 9.77 (s, 1H), 9.47 (d, J=2.8 Hz, 1H), 9.35 (s, 1H), 8.17 (d, J=2.4 Hz, 1H), 7.23 (t, J=53.6 Hz, 1H), 7.10 (s, 1H), 4.41 (d, J=12.4 Hz, 1H), 3.79 (d, J=12.0 Hz, 1H), 2.21-2.27 (m, 1H), 0.81-0.90 (m, 1H), 0.58-0.65 (m, 1H), 0.46-0.54 (m, 2H). LC-MS: m/z 474 [M+H]⁺.

### Method Z4

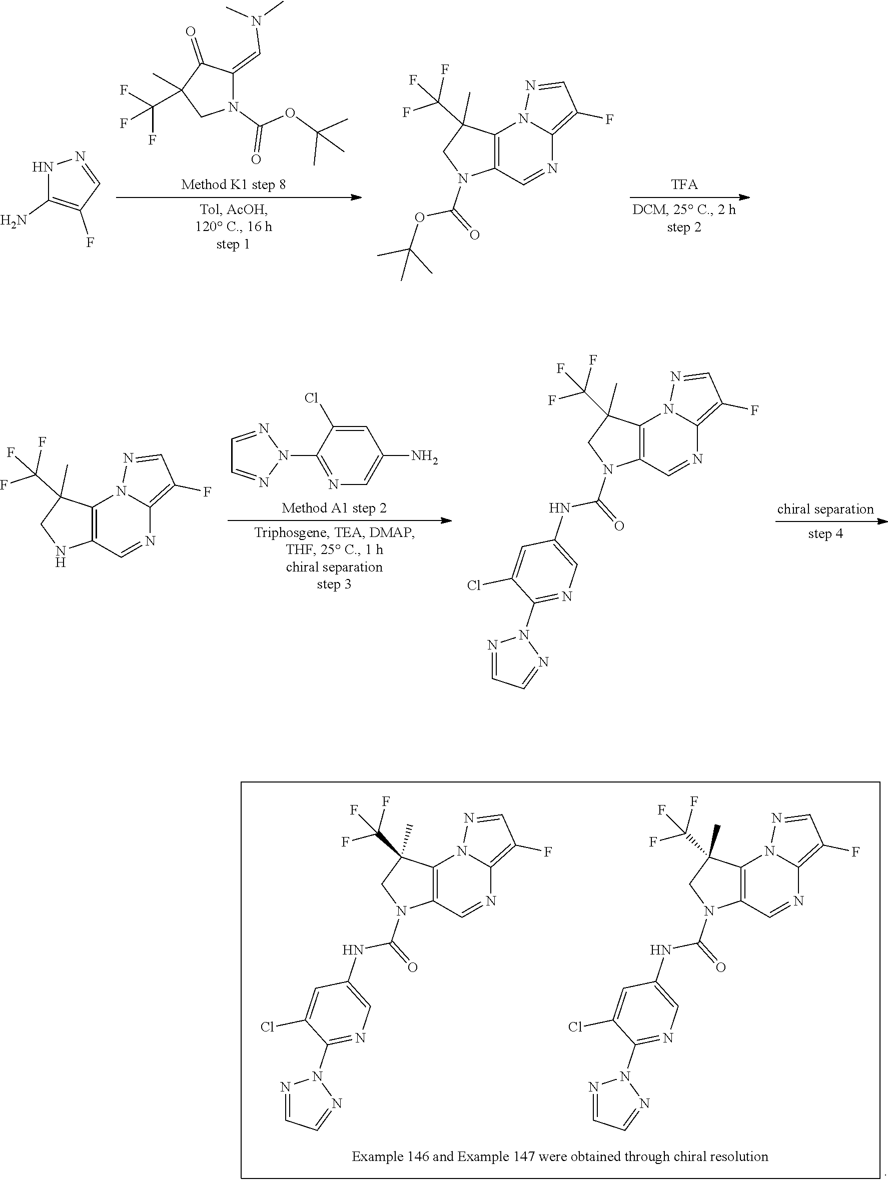

### Examples 146 and 147: Single enantiomers obtained from a racemic mixture containing (R)—N-(5-chloro-6-(2H-1,2,3-triazol-2-yl)pyridin-3-yl)-3-fluoro-8-methyl-8-(trifluoromethyl)-7,8-dihydro-6H-pyrazolo[1,5-a]pyrrolo[2,3-e]pyrimidine-6-carboxamide and (S)—N-(5-chloro-6(2H-1,2,3-triazol-2-yl)pyridin-3-yl)-3-fluoro-1-methyl-8-(trifluoromethyl)-7,8-dihydro-6H-pyrazolo[1,5-a]pyrrolo[2,3-e]pyrimidine-6-carboxamide

### Step 1: tert-butyl 3-fluoro-8-methyl-8-(trifluoromethyl)-7,8-dihydro-6H-pyrazolo[1,5-a]pyrrolo[2,3-e]pyrimidine-6-carboxylate

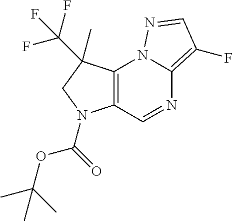 [SMILES: CC(C)(C)OC(=O)N1CC(C)(C(F)(F)F)c2c1cnc1c(F)cnn21]

To a solution of 4-fluoro-1H-pyrazol-5-amine (256 mg, 2.5 mmol) in toluene (10 mL) were added tert-butyl (E)-2-((dimethylamino)methylene)-4-methyl-3-oxo-4-(trifluoromethyl)pyrrolidine-1-carboxylate (Method K1 step 8; 817 mg, 2.5 mmol) and acetic acid (1 mL). The resulting mixture was stirred at 120° C. for 16 h. The resulting solution was concentrated under vacuum. The residue was purified by column chromatography on silica gel using 75% petroleum ether and 25% ethyl acetate as eluents to afford tert-butyl 3-fluoro-8-methyl-8-(trifluoromethyl)-7,8-dihydro-6H-pyrazolo[1,5-a]pyrrolo[2,3-e]pyrimidine-6-carboxylate (350 mg, 38% yield) as a yellow solid. LC-MS: m/z 361 [M+H]⁺.

### Step 2: 3-fluoro-8-methyl-8-(trifluoromethyl)-7,8-dihydro-6H-pyrazolo[1,5-a]pyrrolo[2,3-e]pyrimidine

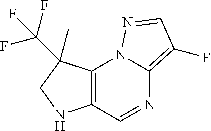 [SMILES: CC1(C(F)(F)F)CNc2cnc3c(F)cnn3c21]

To a solution of tert-butyl 3-fluoro-8-methyl-8-(trifluoromethyl)-7,8-dihydro-6H-pyrazolo[1,5-a]pyrrolo[2,3-e]pyrimidine-6-carboxylate (350 mg, 1.0 mmol) in dichloromethane (5 mL) was added TFA (1 mL). The resulting mixture was stirred for 2 h at 25° C. The mixture was concentrated under vacuum. The reaction mixture was quenched with water (10 mL). The pH was adjusted to 7-8 with saturated aqueous NaHCO₃ solution. The resulting mixture was extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate as eluents to afford 3-fluoro-8-methyl-8-(trifluoromethyl)-7,8-dihydro-6H-pyrazolo[1,5-a]pyrrolo[2,3-e]pyrimidine (100 mg, 40% yield) as a yellow solid. ¹H NMR (300 MHz, DMSO-d₆) δ: 8.35 (s, 1H), 8.15 (d, J=3.3 Hz, 1H), 3.89 (d, J=12.6 Hz, 1H), 3.57 (d, J=12.6 Hz, 1H), 1.81 (s, 3H); LC-MS: m/z 261[M+H]⁺.

### Step 3: N-(5-chloro-6-(2H-1,2,3-triazol-2-yl)pyridin-3-yl)-3-fluoro-8-methyl-8-(trifluoromethyl)-7,8-dihydro-6H-pyrazolo[1,5-a]pyrrolo[2,3-e]pyrimidine-6-carboxamide

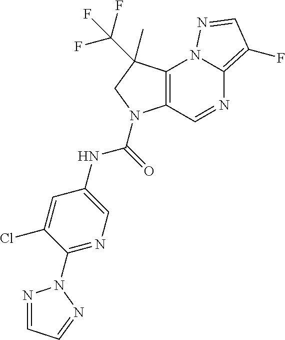 [SMILES: CC1(C(F)(F)F)CN(C(=O)Nc2cnc(-n3nccn3)c(Cl)c2)c2cnc3c(F)cnn3c21]

To a stirred mixture of 5-chloro-6-(triazol-2-yl)pyridin-3-amine (Method A1 step 2; 113 mg, 579.5 μmol) in tetrahydrofuran (8 mL) was added triphosgene (69 mg, 233.1 μmol) and TEA (117 mg, 1.2 mmol). The resulting mixture was stirred for 1 h at 25° C. and then filtered. The resulting filtrate was added to a solution of 3-fluoro-8-methyl-8-(trifluoromethyl)-7,8-dihydro-6H-pyrazolo[1,5-a]pyrrolo[2,3-e]pyrimidine (100 mg, 383.1 μmol) in tetrahydrofuran (1 mL). The mixture was stirred for 1 h at 25° C. The reaction mixture was quenched with water (50 mL). The resulting solution was extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate as eluents to afford crude product. The crude product was purified by Prep-HPLC and the collected fractions were lyophilized to give N-(5-chloro-6-(2H-1,2,3-triazol-2-yl)pyridin-3-yl)-3-fluoro-8-methyl-8-(trifluoromethyl)-7,8-dihydro-6H-pyrazolo[1,5-a]pyrrolo[2,3-e]pyrimidine-6-carboxamide (30 mg, 16% yield) as a yellow solid. LC-MS: m/z 482 [M+H]⁺.

### Step 4: Separation of enantiomers to obtain (S)—N-(5-chloro-6-(2H-1,2,3-triazol-2-yl)pyridin-3-yl)-3-fluoro-8-methyl-8-(trifluoromethyl)-7,8-dihydro-6H-pyrazolo[1,5-a]pyrrolo[2,3-e]pyrimidine-6-carboxamide and (R)—N-(5-chloro-6-(2H-1,2,3-triazol-2-yl)pyridin-3-yl)-3-fluoro-8-methyl-8-(trifluoromethyl)-7,8-dihydro-6H-pyrazolo[1,5-a]pyrrolo[2,3-e]pyrimidine-6-carboxamide

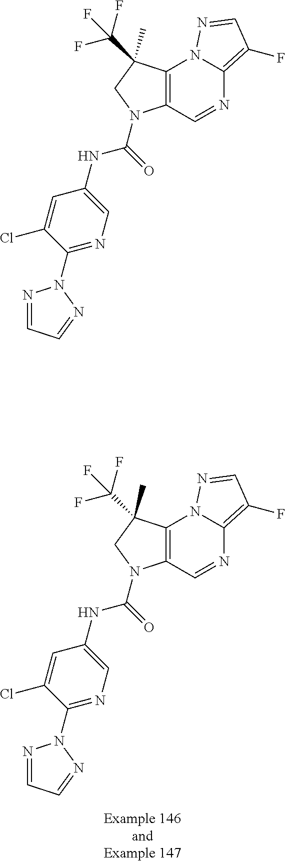 [SMILES: C[C@@]1(C(F)(F)F)CN(C(=O)Nc2cnc(-n3nccn3)c(Cl)c2)c2cnc3c(F)cnn3c21 ; C[C@]1(C(F)(F)F)CN(C(=O)Nc2cnc(-n3nccn3)c(Cl)c2)c2cnc3c(F)cnn3c21]

28 mg of N-(5-chloro-6-(2H-1,2,3-triazol-2-yl)pyridin-3-yl)-3-fluoro-8-methyl-8-(trifluoromethyl)-7,8-dihydro-6H-pyrazolo[1,5-a]pyrrolo[2,3-e]pyrimidine-6-carboxamide was submitted to chiral HPLC purification: Column: CHIRALPAK IA, 2×25 cm, 5 um; Mobile Phase A: Hex(0.5% 2M NH₃-MeOH)—HPLC, Mobile Phase B:EtOH—HPLC; Flow rate: 20 mL/min; Gradient: 10 B to 10 B in 30 min; 220/254 nm; RTL: 20.819; RT2: 25.766; Injection Volume: 0.8 ml; Number Of Runs: 5; The first eluting isomer was concentrated and lyophilized to afford Example 147 (8.8 mg, 5% yield) as a yellow solid. The second eluting isomer was concentrated and lyophilized to afford Example 146 (8.9 mg, 5% yield) a yellow solid. Examples 146 and 147 are enantiomers, but their absolute stereochemistry is not yet known.

Example 146: ¹H NMR (400 MHz, DMSO-d₆) δ 9.71 (br, 1H), 9.33 (s, 1H), 8.75 (d, J=2.4 Hz, 1H), 8.51 (d, J=2.4 Hz, 1H), 8.42 (d, J=3.2 Hz, 1H), 8.18 (s, 2H), 4.86 (d, J=11.6 Hz, 1H), 4.32 (d, J=11.6 Hz, 1H), 2.00 (s, 3H); LC-MS: m/z 482 [M+H]⁺.

Example 147: ¹H NMR (400 MHz, DMSO-d₆) δ 9.71 (br, 1H), 9.33 (s, 1H), 8.75 (d, J=2.4 Hz, 1H), 8.51 (d, J=2.4 Hz, 1H), 8.42 (d, J=3.2 Hz, 1H), 8.18 (s, 2H), 4.86 (d, J=11.6 Hz, 1H), 4.32 (d, J=11.6 Hz, 1H), 2.00 (s, 3H); LC-MS: m/z 482 [M+H]⁺.

### Method A5

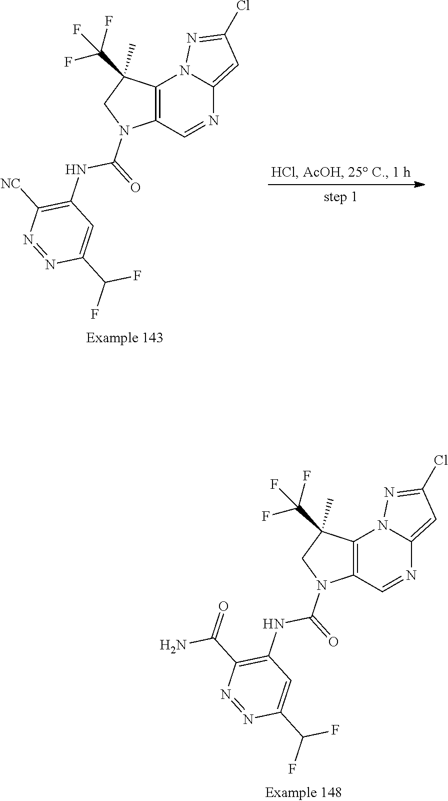 [SMILES: C[C@@]1(C(F)(F)F)CN(C(=O)Nc2cc(C(F)F)nnc2C#N)c2cnc3cc(Cl)nn3c21 ; C[C@@]1(C(F)(F)F)CN(C(=O)Nc2cc(C(F)F)nnc2C(N)=O)c2cnc3cc(Cl)nn3c21]

### Example 148: (R)—N-(3-carbamoyl-6-(difluoromethyl)pyridazin-4-yl)-2-chloro-8-methyl-8-(trifluoromethyl)-7,8-dihydro-6H-pyrazolo[1,5-a]pyrrolo[2,3-e]pyrimidine-6-carboxamide

### Step 1: (R)—N-(3-carbamoyl-6-(difluoromethyl)pyridazin-4-yl)-2-chloro-8-methyl-8-(trifluoromethyl)-7,8-dihydro-6H-pyrazolo[1,5-a]pyrrolo[2,3-e]pyrimidine-6-carboxamide

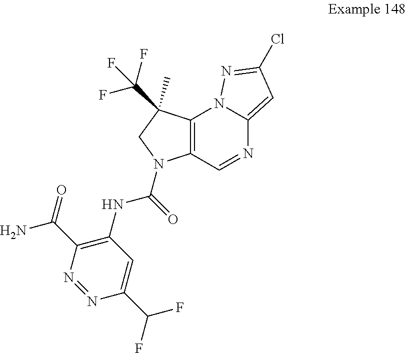 [SMILES: C[C@@]1(C(F)(F)F)CN(C(=O)Nc2cc(C(F)F)nnc2C(N)=O)c2cnc3cc(Cl)nn3c21]

To a stirred solution of (R)-2-chloro-N-(3-cyano-6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide (54 mg, 114.2 μmol) in conc. HCl solution (4 mL) was added acetic acid (4 mL). The reaction mixture was stirred at 25° C. for 1 h. The reaction mixture was concentrated under vacuum. The residue was purified by Prep-HPLC purification and the collected fractions were lyophilized to give (R)—N-(3-carbamoyl-6-(difluoromethyl)pyridazin-4-yl)-2-chloro-8-methyl-8-(trifluoromethyl)-7,8-dihydro-6H-pyrazolo[1,5-a]pyrrolo[2,3-e]pyrimidine-6-carboxamide (13.5 mg, 23.8% yield) as a light-yellow solid.

Example 148: ¹H NMR (300 MHz, DMSO-d₆) δ: 12.73 (br, 1H), 9.40 (s, 1H), 9.22 (br, 1H), 8.83 (s, 1H), 8.51 (br, 1H), 7.35 (t, J=54.0 Hz, 1H), 7.11 (s, 1H), 4.63 (d, J=11.1 Hz, 1H), 4.39 (d, J=11.1 Hz, 1H), 2.01 (s, 3H). LC-MS: m/z 491 [M+H]⁺.

### Method B5

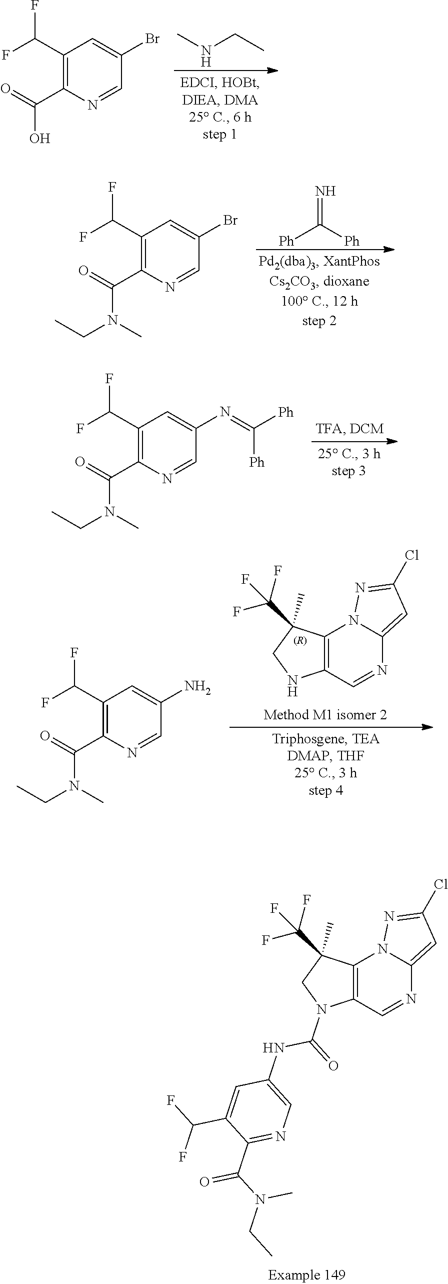

### Example 149: (R)-2-chloro-N-(5-(difluoromethyl)-6-(ethyl(methyl)carbamoyl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 5-bromo-3-(difluoromethyl)-N-ethyl-N-methylpicolinamide

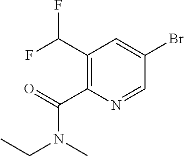 [SMILES: CCN(C)C(=O)c1ncc(Br)cc1C(F)F]

To a stirred solution of 5-bromo-3-(difluoromethyl)picolinic acid (330 mg, 1.3 mmol) in N,N-dimethylacetamide (6 mL) was added N-methylethanamine (93 mg, 1.6 mmol), EDCI (326 mg, 1.7 mmol), HOBt (230 mg, 1.7 mmol) and DIEA (508 mg, 3.9 mmol). The reaction mixture was stirred at 25° C. for 6 h. The reaction mixture was quenched with water (30 mL). The resulting solution was extracted with ethyl acetate (3×30 mL). The combined organic layers were washed with brine (50 mL), dried over anhydrous sodium sulfate and concentrated under vacuum. The residue was purified by column chromatography on silica gel using 60% petroleum ether and 40% ethyl acetate as eluents to afford 5-bromo-3-(difluoromethyl)-N-ethyl-N-methylpicolinamide (240 mg, 61% yield) as a colorless oil. LC-MS: m/z 293 [M+H]⁺.

### Step 2: 3-(difluoromethyl)-5-((diphenylmethylene)amino)-N-ethyl-N-methylpicolinamide

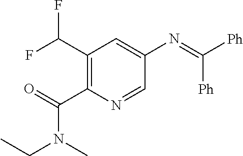 [SMILES: CCN(C)C(=O)c1ncc(N=C(c2ccccc2)c2ccccc2)cc1C(F)F]

To a mixture of 5-bromo-3-(difluoromethyl)-N-ethyl-N-methylpicolinamide (200 mg, 682 μmol) in dioxane (10 mL) was added diphenylmethanimine (185 mg, 1.0 mmol), Pd₂(dba)₃ (62 mg, 68 μmol), Xantphos (39 mg, 68 μmol) and Cs₂CO₃ (667 mg, 2.1 mmol) under nitrogen atmosphere. The resulting mixture was stirred at 100° C. for 12 h. The reaction mixture was concentrated under vacuum. The resulting mixture was diluted with water (10 mL) and extracted with ethyl acetate (3×10 mL). The combined organic layers were dried over anhydrous sodium sulfate and concentrated under vacuum. The residue was purified by column chromatography on silica gel using 75% petroleum ether and 25% ethyl acetate to afford 3-(difluoromethyl)-5-((diphenylmethylene)amino)-N-ethyl-N-methylpicolinamide (250 mg, 77% yield) as a yellow solid. LC-MS: m/z 394 [M+H]⁺.

### Step 3: 5-amino-3-(difluoromethyl)-N-ethyl-N-methylpicolinamide

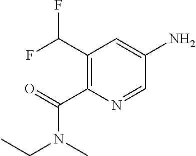 [SMILES: CCN(C)C(=O)c1ncc(N)cc1C(F)F]

To a stirred mixture of 3-(difluoromethyl)-5-((diphenylmethylene)amino)-N-ethyl-N-methylpicolinamide (250 mg, 635 μmol) in dichloromethane (5 mL) was added TFA (1 mL). The resulting mixture was stirred at 25° C. for 3 h. The mixture was concentrated under vacuum. The residue was diluted with saturated aqueous NaHCO₃ solution (10 mL). The resulting solution was extracted with dichloromethane (3×10 mL). The combined organic layers were dried over anhydrous sodium sulfate and concentrated under vacuum. The residue was purified by column chromatography on silica gel using 90% dichloromethane and 10% methanol as eluents to afford 5-amino-3-(difluoromethyl)-N-ethyl-N-methylpicolinamide (100 mg, 67% yield) as a white solid. LC-MS: m/z 230 [M+H]⁺.

### Step 4: (R)-2-chloro-N-(5-(difluoromethyl)-6-(ethyl(methyl)carbamoyl)pyridin-3-yl)-8-methyl-8-(trifluoromethyl)-7,8-dihydro-6H-pyrazolo[1,5-a]pyrrolo[2,3-e]pyrimidine-6-carboxamide

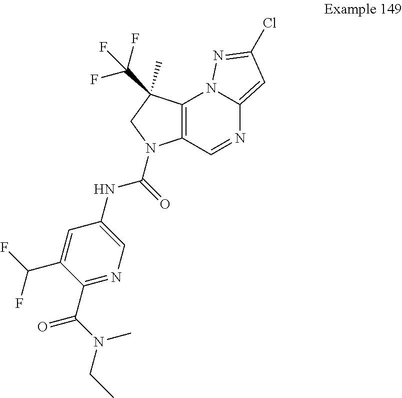 [SMILES: CCN(C)C(=O)c1ncc(NC(=O)N2C[C@@](C)(C(F)(F)F)c3c2cnc2cc(Cl)nn32)cc1C(F)F]

To a stirred solution of 5-amino-3-(difluoromethyl)-N-ethyl-N-methylpicolinamide (20 mg, 87 μmol) in tetrahydrofuran (1 mL) were added triphosgene (16 mg, 52 μmol) and TEA (18 mg, 174 μmol). The resulting mixture was stirred at 25° C. for 1 h and then filtered. The filtrate was added to a solution of Method M1 isomer 2 (24 mg, 87 μmol) in tetrahydrofuran (1 mL). To this solution were added N,N-dimethylpyridin-4-amine (11 mg, 87 μmol) and TEA (18 mg, 174 μmol). The mixture was stirred at 25° C. for 3 h. The mixture was concentrated under vacuum. The residue was purified by Prep-HPLC and the collected fractions were lyophilized to afford (R)-2-chloro-N-(5-(difluoromethyl)-6-(ethyl(methyl)carbamoyl)pyridin-3-yl)-8-methyl-8-(trifluoromethyl)-7,8-dihydro-6H-pyrazolo[1,5-a]pyrrolo[2,3-e]pyrimidine-6-carboxamide (13.6 mg, 29% yield) as a white solid. The enantiomer of Example 149 can be prepared analogously using Method M1 isomer 1.

Example 149: ¹H NMR (400 MHz, DMSO-d₆) δ: 9.56 (s, 1H), 9.35 (s, 1H), 8.92 (d, J=2.0 Hz, 1H), 8.37 (d, J=2.0 Hz, 1H), 7.11 (t, J=55.2 Hz, 1H), 7.07 (s, 1H), 4.87 (d, J=11.2 Hz, 1H), 4.28 (d, J=11.6 Hz, 1H), 3.50 (m, 1H), 3.17 (m, 1H), 2.81-3.00 (m, 3H), 1.99 (s, 3H), 1.05-1.16 (m, 3H). LC-MS: m/z 532 [M+H]⁺.

### Method C5

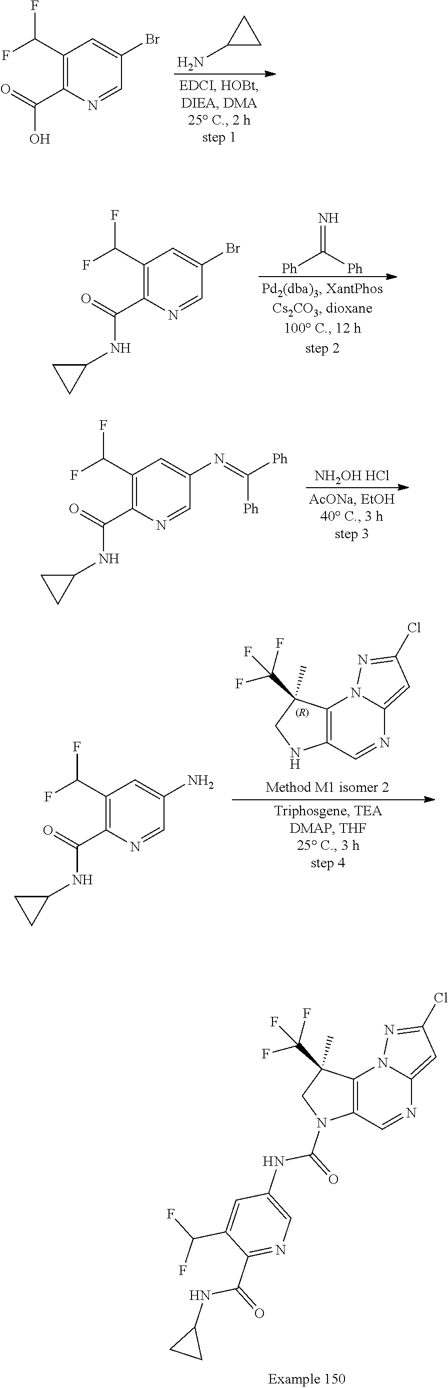

### Example 150: (R)-2-chloro-N-(6-(cyclopropylcarbamoyl)-5-(difluoromethyl)pyridin-3-yl)-8-methyl-S-(trifluoromethyl)-7,8-dihydro-6H-pyrazolo[1,5-a]pyrrolo[2,3-e]pyrimidine-6-carboxamide

### Step 1: 5-bromo-N-cyclopropyl-3-(difluoromethyl)picolinamide

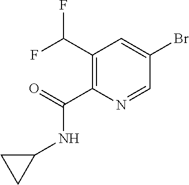 [SMILES: O=C(NC1CC1)c1ncc(Br)cc1C(F)F]

To a stirred solution of 5-bromo-3-(difluoromethyl)picolinic acid (220 mg, 873 μmol) in N,N-dimethylacetamide (4 mL) was added cyclopropanamine (60 mg, 1.1 mmol), EDCI (218 mg, 1.1 mmol), HOBt (153 mg, 1.1 mmol) and DIEA (338 mg, 2.6 mmol). The reaction mixture was stirred at 25° C. for 2 h. The reaction mixture was quenched with water (20 mL). The resulting solution was extracted with ethyl acetate (3×20 mL). The combined organic layers were washed with brine (30 mL), dried over anhydrous sodium sulfate and concentrated under vacuum. The residue was purified by column chromatography on silica gel using 60% petroleum ether and 40% ethyl acetate as eluents to afford 5-bromo-N-cyclopropyl-3-(difluoromethyl)picolinamide (100 mg, 39% yield) as a white solid. LC-MS: m/z 291 [M+H]⁺.

### Step 2: N-cyclopropyl-3-(difluoromethyl)-5-((diphenylmethylene)amino)picolinamide

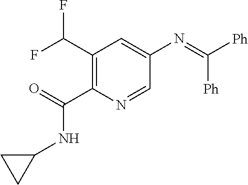 [SMILES: O=C(NC1CC1)c1ncc(N=C(c2ccccc2)c2ccccc2)cc1C(F)F]

To a mixture of 5-bromo-N-cyclopropyl-3-(difluoromethyl)picolinamide (95 mg, 326 μmol) in dioxane (10 mL) was added diphenylmethanimine (89 mg, 490 μmol), Pd₂(dba)₃ (30 mg, 33 μmol), Xantphos (19 mg, 33 μmol) and Cs₂CO₃ (319 mg, 979 μmol) under nitrogen atmosphere. The resulting mixture was stirred at 100° C. for 12 h. The reaction mixture was concentrated under vacuum. The residue was diluted with water (10 mL), and the resulting solution was extracted with ethyl acetate (3×10 mL). The combined organic layers were dried over anhydrous sodium sulfate and concentrated under vacuum. The residue was purified by column chromatography on silica gel using 75% petroleum ether and 25% ethyl acetate to afford N-cyclopropyl-3-(difluoromethyl)-5-((diphenylmethylene)amino)picolinamide (93 mg, 73% yield) as a yellow solid. LC-MS: m/z 392 [M+H]⁺.

### Step 3: 5-amino-N-cyclopropyl-3-(difluoromethyl)picolinamide

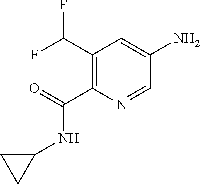 [SMILES: Nc1cnc(C(=O)NC2CC2)c(C(F)F)c1]

To a stirred mixture of N-cyclopropyl-3-(difluoromethyl)-5-((diphenylmethylene)amino)picolinamide (90 mg, 230 μmol) in ethanol (3 mL) were added hydroxylamine hydrochloride (32 mg, 460 μmol) and sodium acetate (47 mg, 575 μmol). The mixture was stirred at 40° C. for 3 h. The resulting mixture was concentrated under vacuum. The residue was diluted with water (10 mL), and the resulting solution was extracted with ethyl acetate (3×10 mL). The combined organic layers were dried over anhydrous sodium sulfate and concentrated under vacuum. The residue was purified by column chromatography on silica gel using 50% petroleum ether and 50% ethyl acetate as eluents to afford 5-amino-N-cyclopropyl-3-(difluoromethyl)picolinamide (30 mg, 50% yield) as a white solid. LC-MS: m/z 228 [M+H]⁺.

### Step 4: (R)-2-chloro-N-(6-(cyclopropylcarbamoyl)-5-(difluoromethyl)pyridin-3-yl)-8-methyl-8-(trifluoromethyl)-7,8-dihydro-6H-pyrazolo[1,5-a]pyrrolo[2,3-e]pyrimidine-6-carboxamide

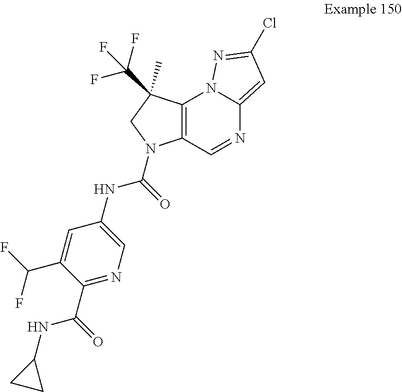 [SMILES: C[C@@]1(C(F)(F)F)CN(C(=O)Nc2cnc(C(=O)NC3CC3)c(C(F)F)c2)c2cnc3cc(Cl)nn3c21]

To a stirred solution of 5-amino-N-cyclopropyl-3-(difluoromethyl)picolinamide (20 mg, 88 μmol) in tetrahydrofuran (1 mL) were added triphosgene (16 mg, 52 μmol) and TEA (18 mg, 176 μmol). The resulting mixture was stirred at 25° C. for 1 h and then filtered. The filtrate was added to a solution of Method M1 isomer 2 (24 mg, 87 μmol) in tetrahydrofuran (1 mL). To this solution were added N,N-dimethylpyridin-4-amine (11 mg, 88 μmol) and TEA (18 mg, 176 μmol). The mixture was stirred at 25° C. for 3 h. The mixture was concentrated under vacuum. The residue was purified by Prep-HPLC and the collected fractions were lyophilized to afford (R)-2-chloro-N-(6-(cyclopropylcarbamoyl)-5-(difluoromethyl)pyridin-3-yl)-8-methyl-8-(trifluoromethyl)-7,8-dihydro-6H-pyrazolo[1,5-a]pyrrolo[2,3-e]pyrimidine-6-carboxamide (17 mg, 36% yield) as a white solid. The enantiomer of Example 150 can be prepared analogously using Method M1 isomer 1.

Example 150: ¹H NMR (300 MHz, DMSO-d₆) δ: 9.61 (s, 1H), 9.34 (s, 1H), 8.96 (d, J=2.1 Hz, 1H), 8.83 (d, J=5.1 Hz, 1H), 8.46 (d, J=2.1 Hz, 1H), 7.89 (t, J=55.5 Hz, 1H), 7.06 (s, 1H), 4.84 (d, J=11.7 Hz, 1H), 4.28 (d, J=11.7 Hz, 1H), 2.85-2.90 (m, 1H), 1.96 (s, 3H), 0.65-0.71 (m, 4H). LC-MS: m/z 530 [M+H]⁺.

### Method D5

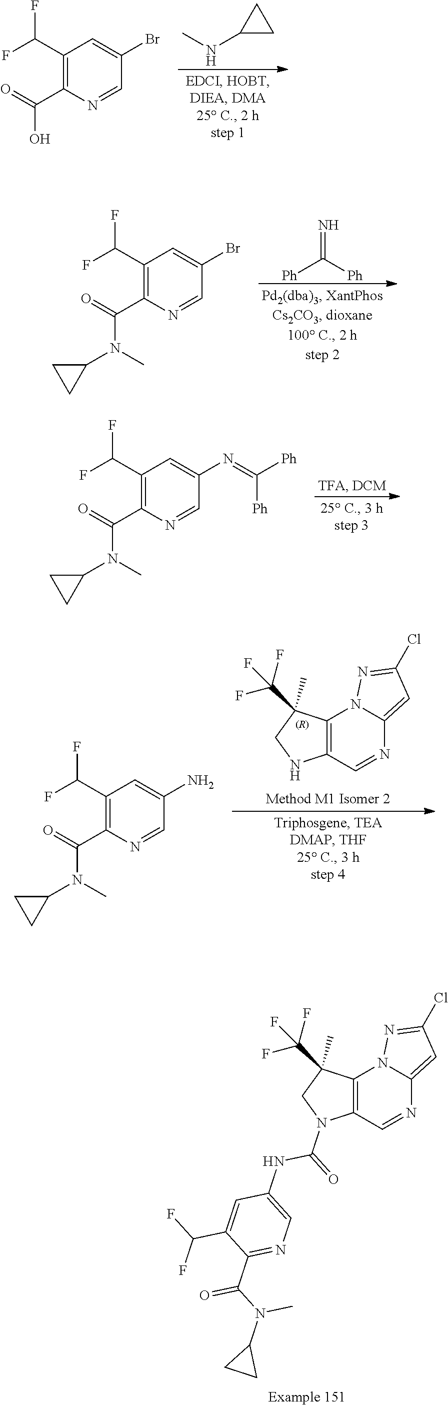

### Example 151 (R)-2-chloro-N-(6-(cyclopropyl(methyl)carbamoyl)-5-(difluoromethyl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 5-bromo-N-cyclopropyl-3-(difluoromethyl)-N-methylpicolinamide

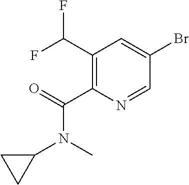 [SMILES: CN(C(=O)c1ncc(Br)cc1C(F)F)C1CC1]

To a stirred solution of 5-bromo-3-(difluoromethyl)picolinic acid (220 mg, 873 μmol) in N,N-dimethylacetamide (5 mL) were added N-methylcyclopropanamine (75 mg, 1.1 mmol), EDCI (218 mg, 1.1 mmol), HOBt (153 mg, 1.1 mmol) and DIEA (338 mg, 2.6 mmol). The reaction mixture was stirred at 25° C. for 2 h. The reaction mixture was quenched with water (20 mL). The resulting solution was extracted with ethyl acetate (3×20 mL). The combined organic layers were washed with brine (50 mL), dried over anhydrous sodium sulfate and concentrated under vacuum. The residue was purified by column chromatography on silica gel using 50% petroleum ether and 50% ethyl acetate as eluents to afford 5-bromo-N-cyclopropyl-3-(difluoromethyl)-N-methylpicolinamide (81 mg, 30% yield) as a white solid. LC-MS: m/z 305 [M+H]⁺.

### Step 2: N-cyclopropyl-3-(difluoromethyl)-5-((diphenylmethylene)amino)-N-methylpicolinamide

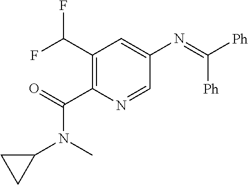 [SMILES: CN(C(=O)c1ncc(N=C(c2ccccc2)c2ccccc2)cc1C(F)F)C1CC1]

To a mixture of 5-bromo-N-cyclopropyl-3-(difluoromethyl)-N-methylpicolinamide (81 mg, 265 μmol) in dioxane (4 mL) was added diphenylmethanimine (48 mg, 265 μmol), Pd₂(dba)₃ (24 mg, 27 μmol), Xantphos (15 mg, 27 μmol) and Cs₂CO₃ (259 mg, 796 μmol) under nitrogen atmosphere. The resulting mixture was stirred at 100° C. for 2 h. The reaction mixture was concentrated under vacuum. The residue was diluted with water (10 mL), and resulting solution was extracted with ethyl acetate (3×10 mL). The combined organic layers were dried over anhydrous sodium sulfate and concentrated under vacuum. The residue was purified by column chromatography on silica gel using 70% petroleum ether and 30% ethyl acetate to afford N-cyclopropyl-3-(difluoromethyl)-5-((diphenylmethylene)amino)-N-methylpicolinamide (81 mg, 30% yield) as a white solid. LC-MS: m/z 406 [M+H]⁺.

### Step 3: 5-amino-N-cyclopropyl-3-(difluoromethyl)-N-methylpicolinamide

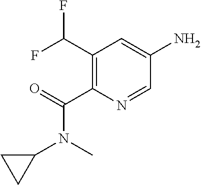 [SMILES: CN(C(=O)c1ncc(N)cc1C(F)F)C1CC1]

To a stirred mixture of N-cyclopropyl-3-(difluoromethyl)-5-((diphenylmethylene)amino)-N-methylpicolinamide (83 mg, 205 μmol) in dichloromethane (5 mL) were added TFA (1 mL). The resulting mixture was stirred at 25° C. for 3 h. The mixture was concentrated under vacuum. The residue was diluted with saturated aqueous NaHCO₃ solution (10 mL). The resulting solution was extracted with dichloromethane (3×10 mL). The combined organic layers were dried over anhydrous sodium sulfate and concentrated under vacuum. The residue was purified by column chromatography on silica gel using 90% dichloromethane and 10% methanol as eluents to afford 5-amino-N-cyclopropyl-3-(difluoromethyl)-N-methylpicolinamide (35 mg, 71% yield) as a white solid. LC-MS: m/z 242 [M+H]⁺.

### Step 4: (R)-2-chloro-N-(6-(cyclopropyl(methyl)carbamoyl)-5-(difluoromethyl)pyridin-3-yl)-8-methyl-8-(trifluoromethyl)-7,8-dihydro-6H-pyrazolo[1,5-a]pyrrolo[2,3-e]pyrimidine-6-carboxamide

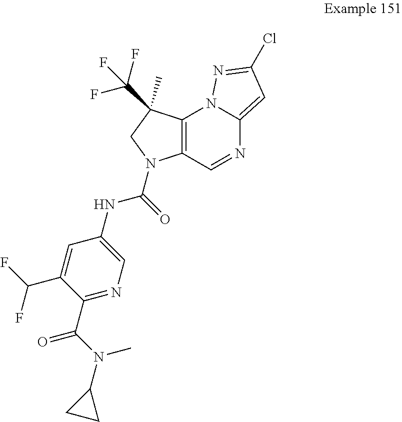 [SMILES: CN(C(=O)c1ncc(NC(=O)N2C[C@@](C)(C(F)(F)F)c3c2cnc2cc(Cl)nn32)cc1C(F)F)C1CC1]

To a stirred solution of 5-amino-N-cyclopropyl-3-(difluoromethyl)-N-methylpicolinamide (35 mg, 145 μmol) in tetrahydrofuran (2 mL) were added triphosgene (26 mg, 87 μmol) and TEA (29 mg, 290 μmol). The resulting mixture was stirred at 25° C. for 1 h and then filtered. The filtrate was added to a solution of Method M1 isomer 2 (40 mg, 145 μmol) in tetrahydrofuran (2 mL). To this solution were added N,N-dimethylpyridin-4-amine (18 mg, 145 μmol) and TEA (29 mg, 290 μmol). The mixture was stirred at 25° C. for 3 h. The mixture was concentrated under vacuum. The residue was purified by Prep-HPLC and the collected fractions were lyophilized to afford (R)-2-chloro-N-(6-(cyclopropyl(methyl)carbamoyl)-5-(difluoromethyl)pyridin-3-yl)-8-methyl-8-(trifluoromethyl)-7,8-dihydro-6H-pyrazolo[1,5-a]pyrrolo[2,3-e]pyrimidine-6-carboxamide (27.2 mg, 42% yield) as a white solid. The enantiomer of Example 151 can be prepared analogously using Method M1 isomer 1.

Example 151: ¹H NMR (300 MHz, DMSO-d₆) δ: 9.60 (s, 1H), 9.36 (s, 1H), 8.94 (s, 1H), 8.38 (s, 1H), 7.14 (t, J=55.2 Hz, 1H), 7.07 (s, 1H), 4.85 (d, J=11.1 Hz, 1H), 4.29 (d, J=11.1 Hz, 1H), 3.03 (s, 3H), 2.75-2.80 (m, 1H), 1.98 (s, 3H), 0.37-0.52 (m, 4H). LC-MS: m/z 544 [M+H]⁺.

### Method E5

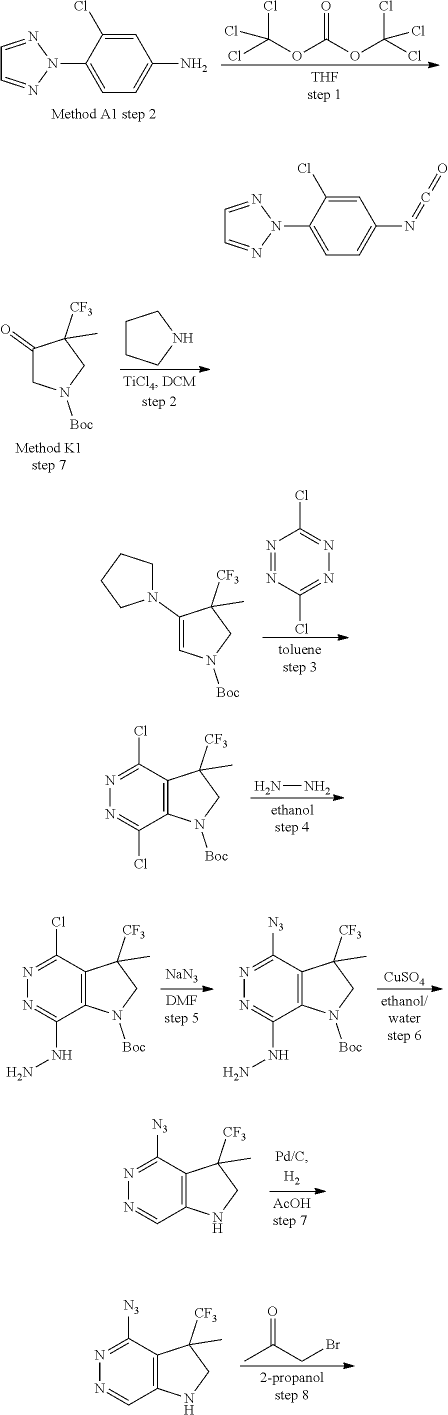

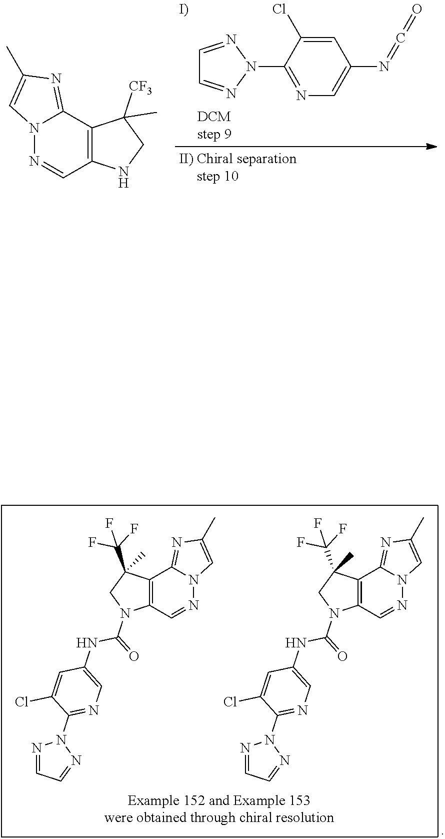

### Examples 152 and 153: Single enantiomers obtained from a racemic mixture containing (S)—N-(5-chloro-6-(2H-1,2,3-triazol-2-yl)pyridin-3-yl)-2,9-dimethyl-9-(trifluoromethyl)-8,9-dihydro-7H-imidazo[1,2-b]pyrrolo[3,2-d]pyridazine-7-carboxamide and (R)—N-(5-chloro-6-(2H-1,2,3-triazol-2-yl)pyridin-3-yl)-2,9-dimethyl-9-(trifluoromethyl)-8,9-dihydro-7H-imidazo[1,2-b]pyrrolo[3,2-d]pyridazine-7-carboxamide

### Step 1: 3-chloro-5-isocyanato-2-(2H-1,2,3-triazol-2-yl)pyridine

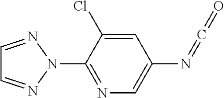 [SMILES: O=C=Nc1cnc(-n2nccn2)c(Cl)c1]

To a solution of 5-chloro-6-(2H-1,2,3-triazol-2-yl)pyridin-3-amine (Method A1 step 2; 100 mg, 0.511 mmol) in tetrahydrofuran (dry) (4.5 mL) was added triethylamine (0.178 mL, 1.278 mmol). After cooling to 0° C. triphosgene (77 mg, 0.261 mmol) was added. The resulting suspension was stirred at 70° C. for 1.5 h. The reaction mixture was cooled down to room temperature and the white precipitate was filtered off. The filter cake was washed with EtOAc (5 mL), and the filtrate was concentrated under reduced pressure to give 3-chloro-5-isocyanato-2-(2H-1,2,3-triazol-2-yl)pyridine (110 mg) as a yellow solid, which was directly used in step 9.

### Step 2: tert-butyl 3-methyl-4-(pyrrolidin-1-yl)-3-(trifluoromethyl)-2,3-dihydro-1H-pyrrole-1-carboxylate

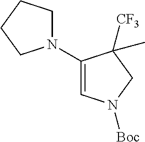 [SMILES: CC(C)(C)OC(=O)N1C=C(N2CCCC2)C(C)(C(F)(F)F)C1]

Under nitrogen atmosphere, tert-butyl 3-methyl-4-oxo-3-(trifluoromethyl)pyrrolidine-1-carboxylate (Method K1 step 7; 1.75 g, 6.55 mmol) was dissolved in pyrrolidine (17.5 mL, 213 mmol) under nitrogen atmosphere, and the reaction solution was cooled to 0° C. Titanium(IV) chloride, 1M in DCM (3.27 mL, 3.27 mmol) was added to the reaction mixture dropwise via a syringe within 30 minutes. The first half the TiCl4 was added at such a rate that the reaction mixture decolourised before the next drop was added. A dark red mixture was obtained. The reaction mixture was allowed to warm to room temperature overnight. The reaction mixture was cooled in an ice-water bath and diluted with diethyl ether (25 mL). This homogeneous mixture was added dropwise to an ice-cold sat. aq. NaHCO₃ solution (150 mL) while stirring. The resulting mixture was extracted with diethyl ether (2×150 mL). The combined organics were washed with water (2×150 mL) and brine (150 mL), dried over Na2SO4, filtered, and concentrated under reduced pressure, dissolved in diethyl ether, transferred into a 100 mL round bottomed flask containing a stirring bar and concentrated in vacuo for 2 hours at 45° C., using an oil pump. This afforded tert-butyl 3-methyl-4-(pyrrolidin-1-yl)-3-(trifluoromethyl)-2,3-dihydro-1H-pyrrole-1-carboxylate (1.97 g) as a yellow oil, which was used directly in the next step.

### Step 3: tert-butyl 4,7-dichloro-3-methyl-3-(trifluoromethyl)-2,3-dihydro-1H-pyrrolo[2,3-d]pyridazine-1-carboxylate

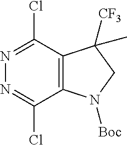 [SMILES: CC(C)(C)OC(=O)N1CC(C)(C(F)(F)F)c2c(Cl)nnc(Cl)c21]

A solution of tert-butyl 3-methyl-4-(pyrrolidin-1-yl)-3-(trifluoromethyl)-2,3-dihydrolH-pyrrole-1-carboxylate (1.97 g, 6.15 mmol) in dry toluene (35 mL) was cooled in an ice-water bath. 3,6-Dichloro-1,2,4,5-tetrazine (0.42 g, 2.78 mmol) was added at once and the resulting red solution was stirred at 0° C. for 30 min. The solvent was removed under reduced pressure and the residue was co-evaporated with heptane three times to remove the excess of tetrazine and purified by flash column chromatography (220 g, 0%-10% ethyl acetate in heptane). This afforded tert-butyl 4,7-dichloro-3-methyl-3-(trifluoromethyl)-2,3-dihydro-1H-pyrrolo[2,3-d]pyridazine-1-carboxylate (800 mg 35% yield) as an orange oil which solidified on standing. ¹H NMR (400 MHz, CDCl₃) δ: 4.64 (d, J=12.6 Hz, 1H), 3.77 (d, J=12.6 Hz, 1H), 1.74 (s, 3H), 1.54 (s, 9H); GC-MS: m/z 271 [M-Boc]⁺.

### Step 4: tert-butyl 4-chloro-7-hydrazineyl-3-methyl-3-(trifluoromethyl)-2,3-dihydro-1H-pyrrolo[2,3-d]pyridazine-1-carboxylate

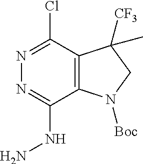 [SMILES: CC(C)(C)OC(=O)N1CC(C)(C(F)(F)F)c2c(Cl)nnc(NN)c21]

tert-butyl 4,7-dichloro-3-methyl-3-(trifluoromethyl)-2,3-dihydro-1H-pyrrolo[2,3-d]pyridazine-1-carboxylate (240 mg, 0.645 mmol) was dissolved in Ethanol (96%, 10 mL) and hydrazine monohydrate (0.491 mL, 6.45 mmol) was added. The reaction mixture was heated to 70° C. for two hours before cooling to ambient temperature overnight. The precipitate was collected by filtration to obtain tert-butyl 4-chloro-7-hydrazineyl-3-methyl-3-(trifluoromethyl)-2,3-dihydrolH-pyrrolo[2,3-d]pyridazine-1-carboxylate (170 mg, 0.462 mmol, 71.7% yield) as a white solid. LC-MS: m/z 368 [M+H]⁺.

### Step 5: tert-butyl 4-azido-7-hydrazineyl-3-methyl-3-(trifluoromethyl)-2,3-dihydro-1H-pyrrolo[2,3-d]pyridazine-1-carboxylate

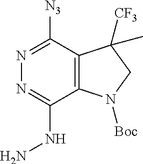 [SMILES: CC(C)(C)OC(=O)N1CC(C)(C(F)(F)F)c2c(N=[N+]=[N-])nnc(NN)c21]

Under nitrogen atmosphere, sodium azide (0.457 g, 7.04 mmol) was added to a solution of tert-butyl 4-chloro-7-hydrazineyl-3-methyl-3-(trifluoromethyl)-2,3-dihydro-1H-pyrrolo[2,3-d]pyridazine-1-carboxylate (1.294 g, 3.52 mmol) in DMF (35 mL). The resulting mixture was stirred at 80° C. overnight. The reaction mixture was concentrated under reduced pressure, dissolved in DMSO and purified by preperative HPLC. The pure fractions were pooled and lyophilised to give tert-butyl 4-azido-7-hydrazineyl-3-methyl-3-(trifluoromethyl)-2,3-dihydro-1H-pyrrolo[2,3-d]pyridazine-1-carboxylate (790 mg, 60% yield). LC-MS: m/z 375 [M+H]⁺.

### Step 6: 4-azido-3-methyl-3-(trifluoromethyl)-2,3-dihydro-1H-pyrrolo[2,3-d]pyridazine

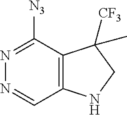 [SMILES: CC1(C(F)(F)F)CNc2cnnc(N=[N+]=[N-])c21]

To a stirred solution of tert-butyl 4-azido-7-hydrazineyl-3-methyl-3-(trifluoromethyl)-2,3-dihydro-1H-pyrrolo[2,3-d]pyridazine-1-carboxylate (374 mg, 0.999 mmol) in ethanol (35 mL) was added water (35 mL) and copper(II) sulfate pentahydrate (1247 mg, 5.00 mmol). The mixture was stirred at 70° C. for 30 hours. The reaction mixture was cooled down to room temperature and filtered over celite. The filter cake was washed with ethanol and the filtrate was concentrated under reduced pressure. The residue was purified by chromatography to obtain 4-azido-3-methyl-3-(trifluoromethyl)-2,3-dihydro-1H-pyrrolo[2,3-d]pyridazine (151 mg, 62% yield). LC-MS: m/z 245 [M+H]⁺.

### Step 7: 3-methyl-3-(trifluoromethyl)-2,3-dihydro-1H-pyrrolo[2,3-d]pyridazin-4-amine

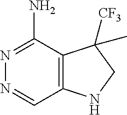

To a solution of 4-azido-3-methyl-3-(trifluoromethyl)-2,3-dihydro-1H-pyrrolo[2,3-d]pyridazine (151 mg, 0.619 mmol) in acetic acid (10 mL) was added 10% palladium on activated carbon (57 mg, 0.054 mmol). The resulting suspension was purged with hydrogen and stirred vigorously for 16 hours at 70° C. under hydrogen atmosphere. The reaction mixture was filtered, the filter was rinsed with ethyl acetate and the filtrate was concentrated under reduced pressure and co-evaporated with toluene and ethyl acetate, to afford 3-methyl-3-(trifluoromethyl)-2,3-dihydro-1H-pyrrolo[2,3-d]pyridazin-4-amine (135 mg, 97% yield) as a yellow solid. LC-MS: m/z 219 [M+H]⁺.

### Step 8: 2,9-dimethyl-9-(trifluoromethyl)-8,9-dihydro-7H-imidazo[1,2-b]pyrrolo[3,2-d]pyridazine

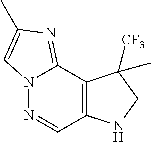

To a solution of 3-methyl-3-(trifluoromethyl)-2,3-dihydro-1H-pyrrolo[2,3-d]pyridazin-4-amine (129 mg, 0.591 mmol) in 2-propanol (30 mL) were subsequently added bromoacetone (0.052 mL, 0.621 mmol) and DIPEA (0.206 mL, 1.182 mmol). The flask was capped with a septum and the reaction mixture was stirred at 50° C. for 6 hours. The reaction mixture was concentrated under reduced pressure and the residue was purified by flash column chromatography to give 2,9-dimethyl-9-(trifluoromethyl)-8,9-dihydro-7H-imidazo[1,2-b]pyrrolo[3,2-d]pyridazine (61 mg, 39% yield) as a yellow solid. H NMR (400 MHz, CDCl₃) δ: 7.97 (s, 1H), 7.59 (s, 1H), 4.07 (d, J=10.9 Hz, 1H), 3.89 (bs, 1H), 3.54 (d, J=10.9 Hz, 1H), 2.46 (s, 3H), 1.86 (s, 3H); LC-MS: m/z 257 [M+H]⁺.

### Step 9: N-(5-chloro-6-(2H-1,2,3-triazol-2-yl)pyridin-3-yl)-2,9-dimethyl-9-(trifluoromethyl)-8,9-dihydro-7H-imidazo[1,2-b]pyrrolo[3,2-d]pyridazine-7-carboxamide

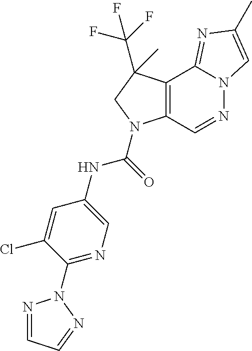 [SMILES: Cc1cn2ncc3c(c2n1)C(C)(C(F)(F)F)CN3C(=O)Nc1cnc(-n2nccn2)c(Cl)c1]

A solution of 2,9-dimethyl-9-(trifluoromethyl)-8,9-dihydro-7H-imidazo[1,2-b]pyrrolo[3,2-d]pyridazine (30 mg, 0.117 mmol) in dichloromethane (1.25 mL) was added to 3-chloro-5-isocyanato-2-(2H-1,2,3-triazol-2-yl)pyridine (40 mg, 0.154 mmol) and the mixture was stirred for 10 minutes. Triethylamine (0.016 mL, 0.117 mmol) was added, and the mixture was stirred for 14 hours. The reaction mixture was concentrated under reduced pressure, dissolved in DMSO, and purified by chromatography to give N-(5-chloro-6-(2H-1,2,3-triazol-2-yl)pyridin-3-yl)-2,9-dimethyl-9-(trifluoromethyl)-8,9-dihydro-7H-imidazo[1,2-b]pyrrolo[3,2-d]pyridazine-7-carboxamide (38 mg) LC-MS: m/z 478 [M+H]⁺.

### Step 10: Separation of enantiomers to obtain (S)—N-(5-chloro-6-(2H-1,2,3-triazol-2-yl)pyridin-3-yl)-2,9-dimethyl-9-(trifluoromethyl)-8,9-dihydro-7H-imidazo[1,2-b]pyrrolo[3,2-d]pyridazine-7-carboxamide and (R)—N-(5-chloro-6-(2H-1,2,3-triazol-2-yl)pyridin-3-yl)-2,9-dimethyl-9-(trifluoromethyl)-8,9-dihydro-7H-imidazo[1,2-b]pyrrolo[3,2-d]pyridazine-7-carboxamide

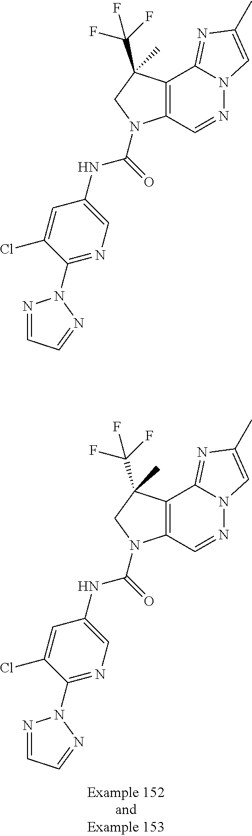 [SMILES: Cc1cn2ncc3c(c2n1)[C@](C)(C(F)(F)F)CN3C(=O)Nc1cnc(-n2nccn2)c(Cl)c1 ; Cc1cn2ncc3c(c2n1)[C@@](C)(C(F)(F)F)CN3C(=O)Nc1cnc(-n2nccn2)c(Cl)c1]

N-(5-chloro-6-(2H-1,2,3-triazol-2-yl)pyridin-3-yl)-2,9-dimethyl-9-(trifluoromethyl)-8,9-dihydro-7H-imidazo[1,2-b]pyrrolo[3,2-d]pyridazine-7-carboxamide (38 mg) was submitted to chiral-SFC (Column: Chiralpak IC, 4.6×100 mm, 5 μm; Mobile Phase A: C02, Mobile Phase B:iPrOH-HPLC; Flow rate: 2.5 mL/min; Gradient: 30 B to 50% B in 5 min; 210-320 nm; RT1:3.572; RT2:3.907). The first eluting isomer was concentrated and lyophilized to afford Example 152 (10.6 mg, 19% yield) and the second eluting isomer was concentrated and lyophilized to afford Example 153 (9.3 mg, 16.6% yield). Examples 152 and 153 are enantiomers, but their absolute stereochemistry is not yet known.

Example 152: ¹H NMR (400 MHz, CDCl₃) δ: 9.31 (s, 1H), 8.59 (d, J=2.4 Hz, 1H), 8.44 (d, J=2.4 Hz, 1H), 7.95 (s, 2H), 7.78 (s, 1H), 6.81 (s, 1H), 4.56 (d, J=10.1 Hz, 1H), 4.04 (d, J=10.1 Hz, 1H), 2.52 (s, 3H), 2.01 (s, 3H). LC-MS: m/z 478 [M+H]⁺.

Example 153: ¹H NMR (400 MHz, CDCl₃) δ: 9.30 (s, 1H), 8.59 (d, J=2.4 Hz, 1H), 8.44 (d, J=2.4 Hz, 1H), 7.95 (s, 2H), 7.78 (s, 1H), 6.80 (s, 1H), 4.56 (d, J=10.1 Hz, 1H), 4.02 (d, J=10.1 Hz, 1H), 2.52 (s, 3H), 2.01 (s, 3H). LC-MS: m/z 478 [M+H]⁺.

### Method F5

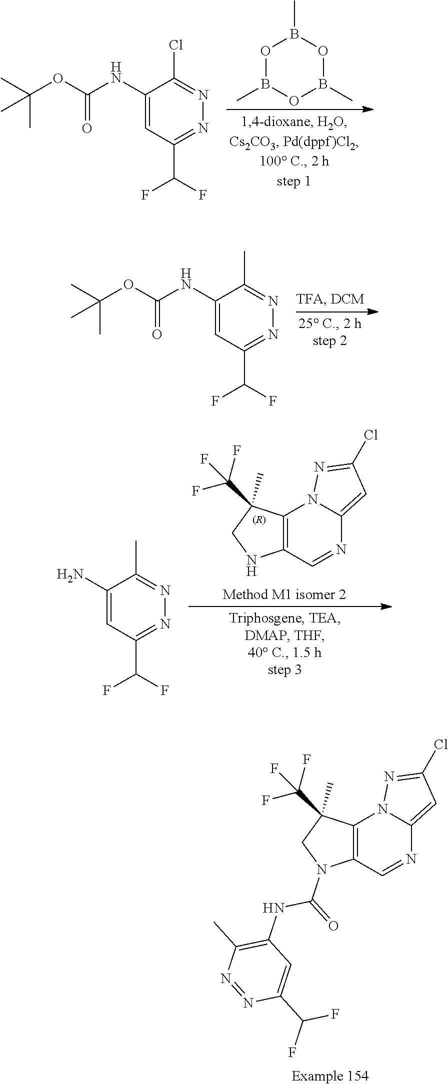

### Example 154: (R)-2-chloro-N-(6-(difluoromethyl)-3-methylpyridazin-4-yl)-8-methyl-8-(trifluoromethyl)-7,8-dihydro-6H-pyrazolo[1,5-a]pyrrolo[2,3-]pyrimidine-6-carboxamide

### Step 1: tert-butyl (6-(difluoromethyl)-3-methylpyridazin-4-yl)carbamate

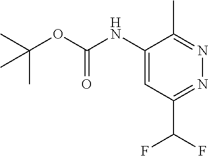 [SMILES: Cc1nnc(C(F)F)cc1NC(=O)OC(C)(C)C]

To a stirred solution of tert-butyl (3-chloro-6-(difluoromethyl)pyridazin-4-yl)carbamate (Method X4, step 2; 520 mg, 1.9 mmol) in dioxane (16 mL) and H₂O (4 mL) were added 2,4,6-trimethyl-1,3,5,2,4,6-trioxatriborinane (26.9 mg, 214.5 μmol), Pd(dppf)Cl₂ (8.8 mg, 10.7 pol) and Cs₂CO₃ (104.9 mg, 321.8 μmol). The mixture was stirred at 100° C. for 2 h under nitrogen atmosphere. The reaction mixture was cooled to 25° C. and concentrated under vacuum. The residue was purified by column chromatography on silica gel using 50% petroleum ether and 50% ethyl acetate as eluents to afford tert-butyl (6-(difluoromethyl)-3-methylpyridazin-4-yl)carbamate (300 mg, 62% yield) as a yellow solid. ¹H NMR (300 MHz, DMSO-d₆) δ: 9.38 (br, 1H), 8.26 (s, 1H), 7.17 (t, J=54.0 Hz, 1H), 2.68 (s, 3H), 1.54 (s, 9H). LC-MS: m/z 260 [M+H]⁺.

### Step 2: 6-(difluoromethyl)-3-methylpyridazin-4-amine

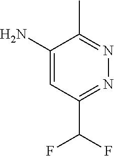

To a stirred solution of tert-butyl (6-(difluoromethyl)-3-methylpyridazin-4-yl)carbamate (270 mg, 1.0 mmol) in dichloromethane (9 mL) was added TFA (3 mL). The reaction mixture was stirred at 25° C. for 2 h and concentrated under vacuum. The residue was diluted with saturated aqueous NaHCO₃ solution (40 mL). The resulting solution was extracted with ethyl acetate (3×50 mL). The combined organic layers were dried over anhydrous sodium sulfate and concentrated under vacuum. The residue was purified by column chromatography on silica gel using 95% dichloromethane and 5% methanol as eluents to afford 6-(difluoromethyl)-3-methylpyridazin-4-amine (130 mg, 78.8% yield) as an off-white solid. ¹H NMR (400 MHz, DMSO-d₆) δ: 6.92 (t, J=54.8 Hz, 1H), 6.81 (s, 1H), 6.58 (br, 2H), 2.42 (s, 3H). LC-MS: m/z 160 [M+H]⁺.

### Step 3: (R)-2-chloro-N-(6-(difluoromethyl)-3-methylpyridazin-4-yl)-8-methyl-8-(trifluoromethyl)-7,8-dihydro-6H-pyrazolo[1,5-a]pyrrolo[2,3-e]pyrimidine-6-carboxamide

To a stirred solution of 6-(difluoromethyl)-3-methylpyridazin-4-amine (40 mg, 251.4 μmol) in tetrahydrofuran (8 mL) were added bis(trichloromethyl) carbonate (44.8 mg, 150.8 μmol) and TEA (38.2 mg, 377.0 μmol). The resulting mixture was stirred at 40° C. for 0.5 h and then filtered. The resulting filtrate was added to a solution of Method M1 isomer 2 (55.6 mg, 201.1 μmol) in tetrahydrofuran (2 mL). To this solution were then added TEA (254.4 mg, 2.5 mmol) and N,N-dimethylpyridin-4-amine (61.4 mg, 502.7 μmol). The mixture was stirred at 40° C. for 1 h. The solvent was concentrated under vacuum. The residue was purified by Prep-TLC using 96% dichloromethane and 4% methanol as eluents to afford 90 mg of the crude product which was purified by Prep-HPLC purification and the collected fractions were lyophilized to give (R)-2-chloro-N-(6-(difluoromethyl)-3-methylpyridazin-4-yl)-8-methyl-8-(trifluoromethyl)-7,8-dihydro-6H-pyrazolo[1,5-a]pyrrolo[2,3-e]pyrimidine-6-carboxamide (25.6 mg, 21.8% yield) as a

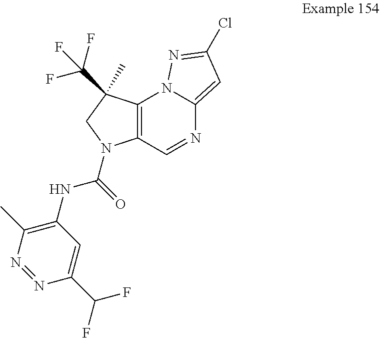 [SMILES: Cc1nnc(C(F)F)cc1NC(=O)N1C[C@@](C)(C(F)(F)F)c2c1cnc1cc(Cl)nn21]

white solid. The enantiomer of Example 154 can be prepared analogously using Method M isomer 1.

Example 154: ¹H NMR (300 MHz, DMSO-d₆) δ: 9.32 (s, 1H), 8.88 (br, 1H), 8.24 (s, 1H), 7.24 (t, J=54.1 Hz, 1H), 7.09 (s, 1H), 4.96 (d, J=11.7 Hz, 1H), 4.39 (d, J=11.7 Hz, 1H), 2.76 (s, 3H), 1.99 (s, 3H). LC-MS: m/z 462 [M+H]⁺.

### Method G5

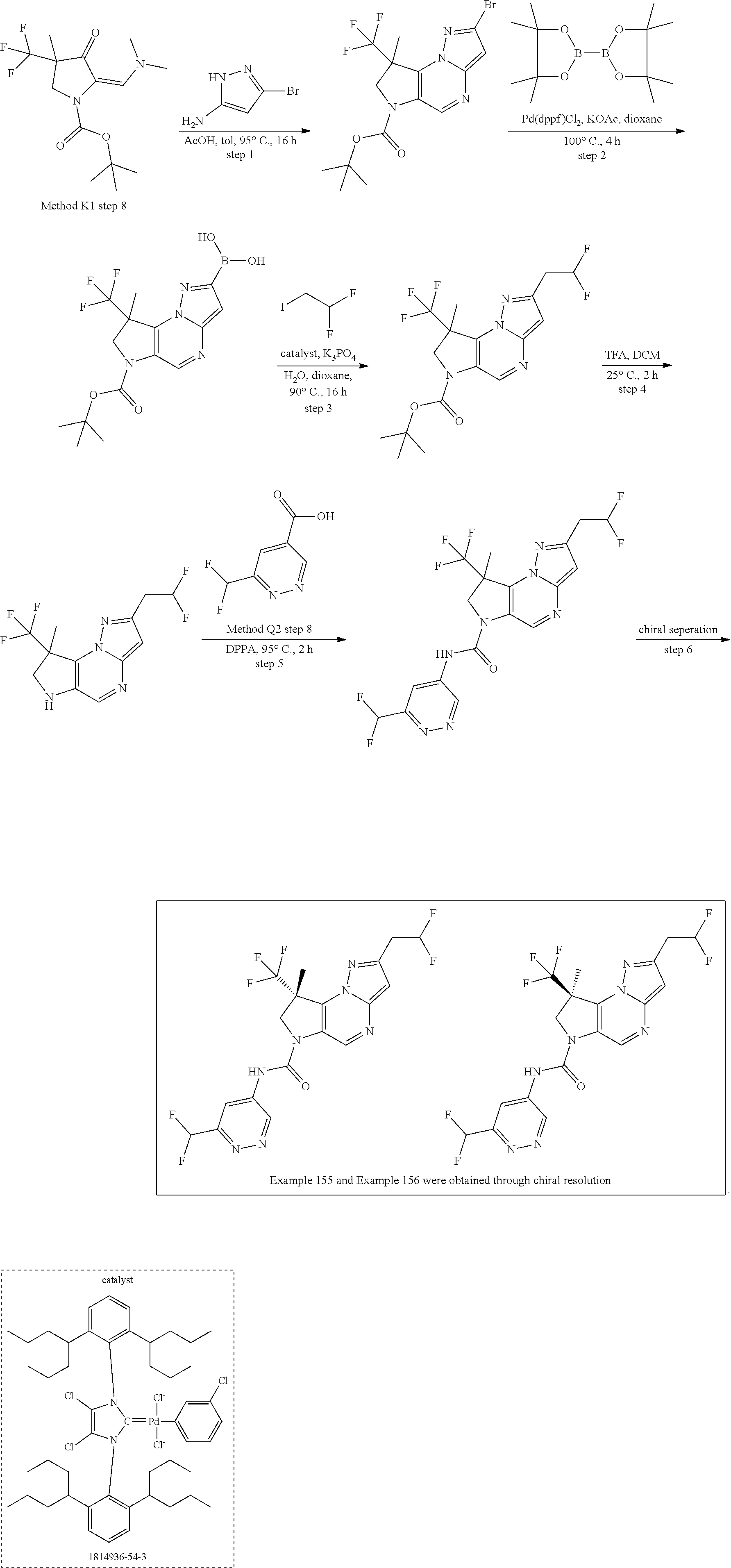

### Examples 155 and 156: Single enantiomers obtained from a racemic mixture containing (S)-2-(2,2-difluoroethyl)-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide and (R)-2-(2,2-difluoroethyl)-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide

### Step 1: tert-butyl 2-bromo-8-methyl-8-(trifluoromethyl)-7,8-dihydro-6H-pyrazolo[1,5-a]pyrrolo[2,3-e]pyrimidine-6-carboxylate

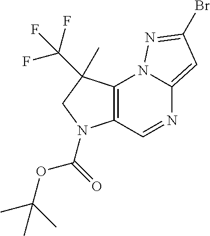 [SMILES: CC(C)(C)OC(=O)N1CC(C)(C(F)(F)F)c2c1cnc1cc(Br)nn21]

To a stirred solution of tert-butyl (E)-2-((dimethylamino)methylene)-4-methyl-3-oxo-4-(trifluoromethyl)pyrrolidine-1-carboxylate (Method K1 step 8; 7 g, 21.7 mmol) in toluene (100 mL) were added 3-bromo-1H-pyrazol-5-amine (3.5 g, 21.7 mmol) and acetic acid (10 mL). The mixture was stirred at 95° C. for 16 h. The reaction mixture was cooled to 25° C. and concentrated under vacuum. The residue was diluted with saturated aqueous NaHCO₃ solution (100 mL). The resulting mixture was extracted with ethyl acetate (3×100 mL). The combined organic layers were dried over anhydrous sodium sulfate and concentrated under vacuum. The residue was purified by column chromatography using 75% petroleum ether and 25% ethyl acetate as eluents to afford tert-butyl 2-bromo-8-methyl-8-(trifluoromethyl)-7,8-dihydro-6H-pyrazolo[1,5-a]pyrrolo[2,3-e]pyrimidine-6-carboxylate (2.6 g, 28% yield) as a yellow oil. LC-MS: m/z 421 [M+H]⁺.

### Step 2: (6-(tert-butoxycarbonyl)-8-methyl-8-(trifluoromethyl)-7,8-dihydro-6H-pyrazolo[1,5-a]pyrrolo[2,3-e]pyrimidin-2-yl)boronic acid

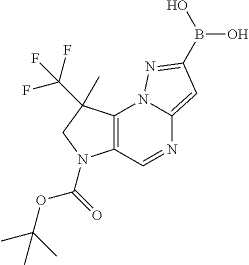 [SMILES: CC(C)(C)OC(=O)N1CC(C)(C(F)(F)F)c2c1cnc1cc(B(O)O)nn21]

To a stirred solution of tert-butyl 2-bromo-8-methyl-8-(trifluoromethyl)-7,8-dihydro-6H-pyrazolo[1,5-a]pyrrolo[2,3-e]pyrimidine-6-carboxylate (2.6 g, 6.2 mmol) and 4,4,5,5-tetramethyl-2-(4,4,5,5-tetramethyl-1,3,2-dioxaborolan-2-yl)-1,3,2-dioxaborolane (1.9 g, 7.4 mmol) in dioxane (100 mL) were added Pd(dppf)Cl₂ (903 mg, 1.2 mmol) and potassium acetate (1.8 g, 18.5 mmol) under nitrogen atmosphere. The mixture was stirred at 100° C. for 16 h. The mixture was cooled to 25° C. The resulting mixture was diluted with ethyl acetate (200 mL). The resulting mixture was filtered. The filter cake was washed with ethyl acetate (3×200 ML). The filtrate was concentrated under vacuum. The residue was purified by column chromatography on silica gel using 75% petroleum ether and 25% ethyl acetate to afford (6-(tert-butoxycarbonyl)-8-methyl-8-(trifluoromethyl)-7,8-dihydro-6H-pyrazolo[1,5-a]pyrrolo[2,3-e]pyrimidin-2-yl)boronic acid (2 g, 67.1% yield) as a yellow oil. LC-MS: m/z 387 [M+H]⁺.

### Step 3: tert-butyl 2-(2,2-difluoroethyl)-8-methyl-8-(trifluoromethyl)-7,8-dihydro-6H-pyrazolo[1,5-a]pyrrolo[2,3-e]pyrimidine-6-carboxylate

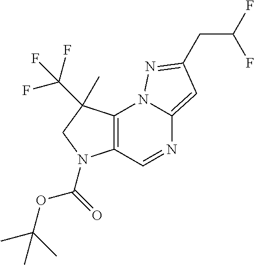 [SMILES: CC(C)(C)OC(=O)N1CC(C)(C(F)(F)F)c2c1cnc1cc(CC(F)F)nn21]

To a stirred solution of (6-(tert-butoxycarbonyl)-8-methyl-8-(trifluoromethyl)-7,8-dihydro-6H-pyrazolo[1,5-a]pyrrolo[2,3-e]pyrimidin-2-yl)boronic acid (1 g, 2.6 mmol) in dioxane (20 mL) and water (4 mL) were added Palladium, [1,3-bis[2,6-bis(1-propylbutyl)phenyl]-4,5-dichloro-1,3-dihydro-2H-imidazol-2-ylidene]dichloro(3-chloropyridine-κN)—, (SP-4-1)-(239 mg, 258.9 μmol), 1,1-difluoro-2-iodo-ethane (5 g, 26.0 mmol) and potassium phosphate tribasic (1.1 g, 5.2 mmol). The reaction mixture was stirred at 90° C. for 16 h under nitrogen atmosphere. The reaction was cooled to 25° C. and concentrated under vacuum. The residue was diluted with water (100 mL), and the resulting mixture was extracted with ethyl acetate (3×200 mL). The combined organic layers were dried over anhydrous sodium sulfate and concentrated under vacuum. The residue was purified by column chromatography using 75% petroleum ether and 25% ethyl acetate as eluents to afford tert-butyl 2-(2,2-difluoroethyl)-8-methyl-8-(trifluoromethyl)-7,8-dihydro-6H-pyrazolo[1,5-a]pyrrolo[2,3-e]pyrimidine-6-carboxylate (400 mg, 28.9% yield) as a yellow oil. LC-MS: m/z 407 [M+H]⁺.

### Step 4: 2-(2,2-difluoroethyl)-8-methyl-8-(trifluoromethyl)-7,8-dihydro-6H-pyrazolo[1,5-a]pyrrolo[2,3-e]pyrimidine

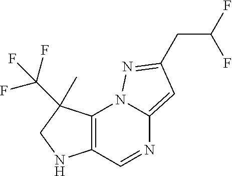 [SMILES: CC1(C(F)(F)F)CNc2cnc3cc(CC(F)F)nn3c21]

To a mixture of tert-butyl 2-(2,2-difluoroethyl)-8-methyl-8-(trifluoromethyl)-7,8-dihydro-6H-pyrazolo[1,5-a]pyrrolo[2,3-e]pyrimidine-6-carboxylate (400 mg, 984.3 μmol) in dichloromethane (25 mL) was added TFA (5 mL). The mixture was stirred at 25° C. for 2 h. The mixture was concentrated under vacuum. The residue was diluted with saturated aqueous NaHCO₃ solution (20 mL), and the resulting mixture was extracted with dichloromethane (3×100 mL). The combined organic layers were dried over anhydrous sodium sulfate and concentrated under vacuum. The residue was purified by Prep-TLC using 97% dichloromethane and 3% methanol as eluents to give 2-(2,2-difluoroethyl)-8-methyl-8-(trifluoromethyl)-7,8-dihydro-6H-pyrazolo[1,5-a]pyrrolo[2,3-e]pyrimidine (230 mg, 63.3% yield) as a brown solid. LC-MS: m/z 307 [M+H]⁺.

### Step 5: 2-(2,2-difluoroethyl)-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide

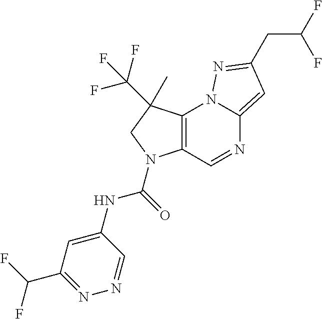 [SMILES: CC1(C(F)(F)F)CN(C(=O)Nc2cnnc(C(F)F)c2)c2cnc3cc(CC(F)F)nn3c21]

To a stirred solution of 2-(2,2-difluoroethyl)-8-methyl-8-(trifluoromethyl)-7,8-dihydro-6H-pyrazolo[1,5-a]pyrrolo[2,3-e]pyrimidine (200 mg, 653.1 μmol) and 6-(difluoromethyl)pyridazine-4-carboxylic acid (Method Q2 step 8; 114 mg, 653.1 μmol) in dioxane (10 mL) were added DPPA (191 mg, 783.7 μmol) and TEA (331 mg, 3.3 mmol). The resulting mixture was stirred at 100° C. for 2 h. After cooled to 25° C., the reaction mixture was concentrated under vacuum. The residue was purified by column chromatography on silica gel using 70% petroleum ether and 30% ethyl acetate as eluents to afford the crude product which was submitted to Prep-HPLC purification and the collected fractions were lyophilized to give 2-(2,2-difluoroethyl)-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide (60 mg 19.1% yield) as a white solid. LC-MS: m/z 478 [M+H]⁺.

### Step 6: Separation of enantiomers to obtain (S)-2-(2,2-difluoroethyl)-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide and (R)-2-(2,2-difluoroethyl)-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide

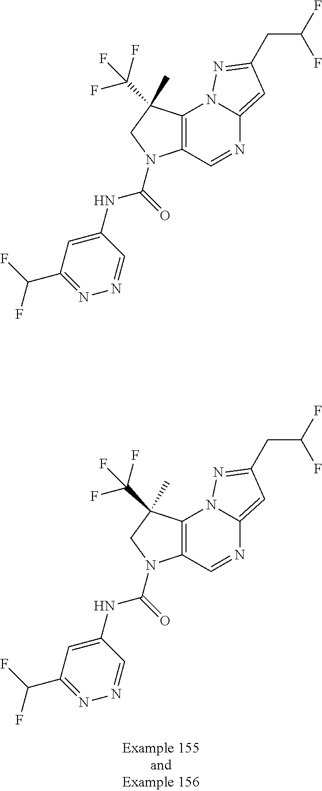 [SMILES: C[C@]1(C(F)(F)F)CN(C(=O)Nc2cnnc(C(F)F)c2)c2cnc3cc(CC(F)F)nn3c21 ; C[C@@]1(C(F)(F)F)CN(C(=O)Nc2cnnc(C(F)F)c2)c2cnc3cc(CC(F)F)nn3c21]

2-(2,2-difluoroethyl)-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide (55 mg, 117.5 μmol) was submitted to chiral-HPLC: Column: Lux Sum Cellulose-2, 2.12×25 cm, 5 μm; Mobile Phase A:Hex(0.5% 2M NH₃-MeOH)—HPLC, Mobile Phase B:EtOH—HPLC; Flow rate: 20 mL/min; Gradient: 50 B to 50 B in 19 min; 220/254 nm; RT1:8.682; RT2:17.225; Injection Volume: 3 ml; Number Of Runs: 3. The first eluting isomer was concentrated and lyophilized to afford Example 155 (17.5 mg, 7.42% yield) as an off-white solid and the second eluting isomer was concentrated and lyophilized to afford Example 156 (15.5 mg, 6% yield) as an off-white solid. Examples 155 and 156 are enantiomers, but their absolute stereochemistry is not yet known.

Example 155: ¹H NMR (400 MHz, DMSO-d₆) δ: 9.90 (br, 1H), 9.51 (d, J=2.4 Hz, 1H), 9.29 (s, 1H), 8.21 (d, J=2.4 Hz, 1H), 7.24 (t, J=54.4 Hz, 1H), 6.86 (s, 1H), 6.44 (t, J=56.0 Hz, 1H), 4.86 (d, J=11.2 Hz, 1H), 4.30 (d, J=11.2 Hz, 1H), 3.51-3.41 (m, 2H), 2.01 (s, 3H). LC-MS: m/z 478 [M+H]⁺.

Example 156: ¹H NMR (400 MHz, DMSO-d₆) δ: 9.91 (br, 1H), 9.52 (d, J=2.8 Hz, 1H), 9.29 (s, 1H), 8.22 (d, J=2.4 Hz, 1H), 7.24 (t, J=54.4 Hz, 1H), 6.86 (s, 1H), 6.44 (t, J=56.0 Hz, 1H), 4.86 (d, J=11.2 Hz, 1H), 4.31 (d, J=11.6 Hz, 1H), 3.51-3.41 (m, 2H), 2.01 (s, 3H). LC-MS: m/z 478 [M+H]⁺.

### Method H5

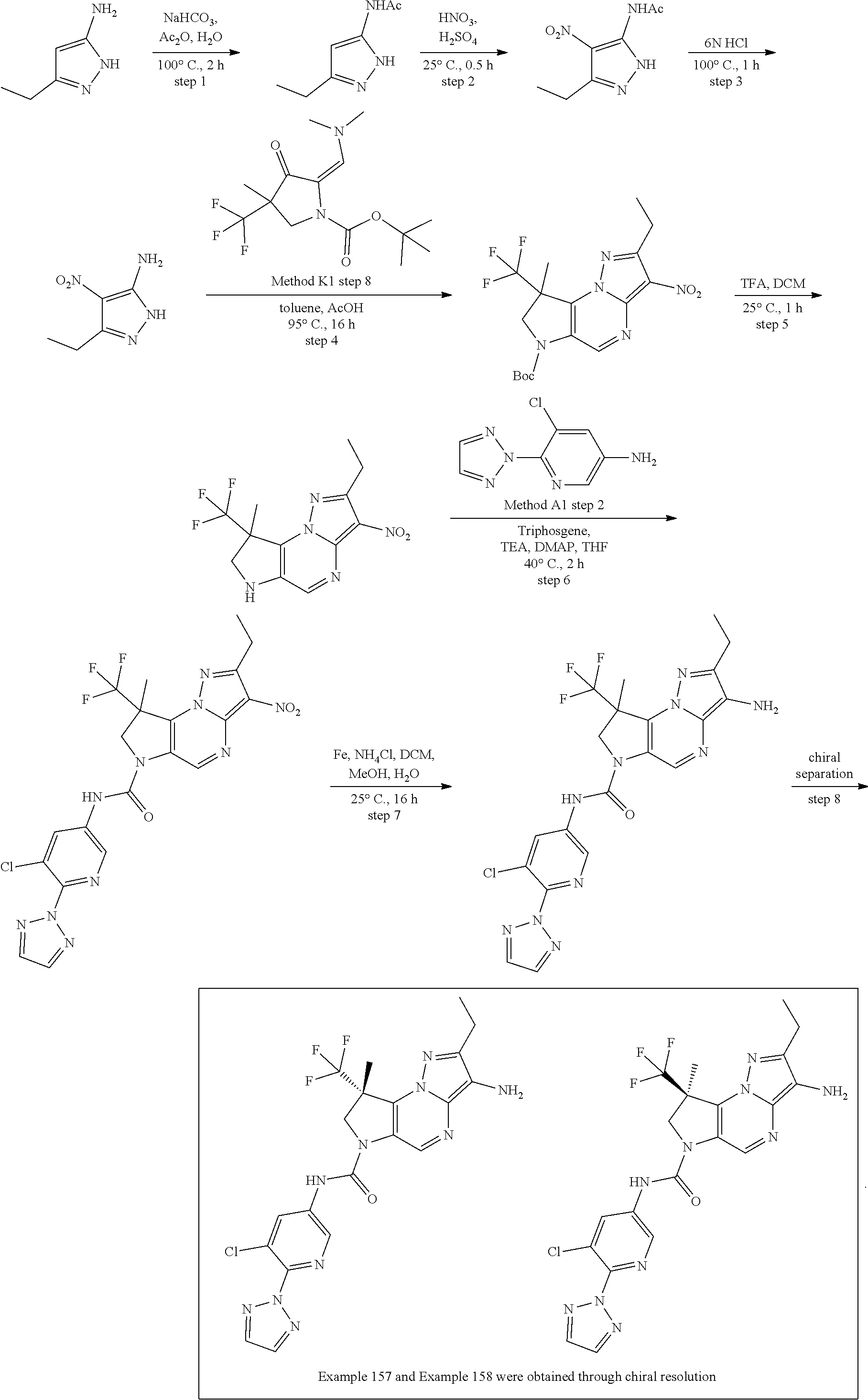

### Examples 157 and 158: Single enantiomers obtained from a racemic mixture containing (S)-3-amino-N-(5-chloro-6-(2H-1,2,3-triazol-2-yl)pyridin-3-yl)-2-ethyl-8-methyl-8-(trifluoromethyl)-7,8-dihydro-6H-pyrazolo[1,5-a]pyrrolo[2,3-e]pyrimidine-6-carboxamide and (R)-3-amino-N-(5-chloro-6-(2H-1,2,3-triazol-2-yl)pyridin-3-yl)-2-ethyl-1-methyl-8-(trifluoromethyl)-7,8-dihydro-6H-pyrazolo[1,5-a]pyrrolo[2,3-e]pyrimidine-6-carboxamide

### Step 1: N-(3-ethyl-1H-pyrazol-5-yl)acetamide

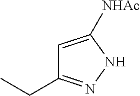

To a stirred solution of 3-ethyl-1H-pyrazol-5-amine (10.0 g, 90.0 mmol) in water (100 mL) were added NaHCO₃ (22.7 g, 270.2 mmol) and acetic anhydride (18.3 g, 179.4 mmol) slowly at 25° C. The reaction mixture was stirred at 100° C. for 2 h. The reaction mixture was stirred at 25° C. for 16 h. The solid was collected to afford N-(3-ethyl-1H-pyrazol-5-yl)acetamide (5.3 g, 38% yield) as a white solid. LC-MS: m/z 154 [M+H]⁺.

### Step 2: N-(3-ethyl-4-nitro-1H-pyrazol-5-yl)acetamide

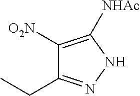

To a stirred solution of N-(3-ethyl-1H-pyrazol-5-yl)acetamide (7.37 g, 48.1 mmol) in concentrated sulfuric acid (17 mL) was added fuming nitric acid (3.03 g, 48.1 mmol) dropwise at 0° C. The mixture was stirred at 25° C. for 30 min. The resulting mixture was diluted with ice water (50 mL). The precipitated solids were collected by filtration, and the filter cake was washed with water (2×20 mL). This resulted in N-(3-ethyl-4-nitro-1H-pyrazol-5-yl)acetamide (5.4 g, 56% yield) as a white solid. LC-MS: m/z 199 [M+H]⁺.

### Step 3: 3-ethyl-4-nitro-1H-pyrazol-5-amine

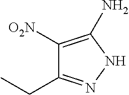

N-(3-ethyl-4-nitro-1H-pyrazol-5-yl)acetamide (5.40 g, 27.2 mmol) was dissolved in water (10 mL) and concentrated HCl (10 mL). The mixture was stirred at 100° C. for 1 h. After cooled to 25° C., the reaction mixture was concentrated. The residue was diluted with tert-Butyl methyl ether (100 mL). The precipitated solids were collected by filtration, and the filter cake washed with tert-Butyl methyl ether (2×50 mL). This resulted in 3-ethyl-4-nitro-1H-pyrazol-5-amine (3.8 g, 89% yield) as a yellow solid. LC-MS: m/z 157 [M+H]⁺.

### Step 4: tert-butyl 2-ethyl-8-methyl-3-nitro-8-(trifluoromethyl)-7,8-dihydro-6H-pyrazolo[1,5-a]pyrrolo[2,3-e]pyrimidine-6-carboxylate

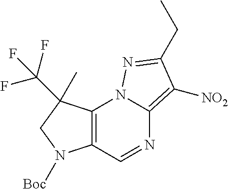 [SMILES: CCc1nn2c3c(cnc2c1[N+](=O)[O-])N(C(=O)OC(C)(C)C)CC3(C)C(F)(F)F]

To a stirred solution of 3-ethyl-4-nitro-1H-pyrazol-5-amine (500 mg, 3.2 mmol) and tert-butyl (E)-2-((dimethylamino)methylene)-4-methyl-3-oxo-4-(trifluoromethyl)pyrrolidine-1-carboxylate (Method K1 step 8; 1.03 g, 3.2 mmol) in toluene (20 mL) was added acetic acid (2 mL). The mixture was stirred at 95° C. for 16 h. After cooled to 25° C., the reaction mixture was concentrated under vacuum. The residue was purified by column chromatography using 50% petroleum ether and 50% ethyl acetate as eluents to afford tert-butyl 2-ethyl-8-methyl-3-nitro-8-(trifluoromethyl)-7,8-dihydro-6H-pyrazolo[1,5-a]pyrrolo[2,3-e]pyrimidine-6-carboxylate (240 mg, 18% yield) as a yellow solid. ¹H NMR (400 MHz, DMSO-d₆) δ: 9.39 (s, 1H), 4.38 (d, J=12.0 Hz, 1H), 4.06 (d, J=12.0 Hz, 1H), 3.16 (q, J=7.6 Hz, 2H), 1.93 (s, 3H), 1.54 (s, 9H), 1.30 (t, J=8.0 Hz, 3H). LC-MS: m/z 416 [M+H]⁺.

### Step 5: 2-ethyl-8-methyl-3-nitro-8-(trifluoromethyl)-7,8-dihydro-6H-pyrazolo[1,5-a]pyrrolo[2,3-e]pyrimidine

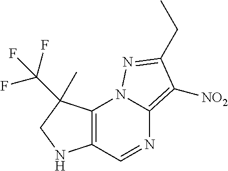 [SMILES: CCc1nn2c3c(cnc2c1[N+](=O)[O-])NCC3(C)C(F)(F)F]

To a stirred solution of tert-butyl 2-ethyl-8-methyl-3-nitro-8-(trifluoromethyl)-7,8-dihydro-6H-pyrazolo[1,5-a]pyrrolo[2,3-e]pyrimidine-6-carboxylate (220 mg, 534.2 μmol) in dichloromethane (5 mL) was added TFA (1 mL) at 0° C. The mixture was stirred at 25° C. for 1 h. The resulting mixture was concentrated under vacuum. The residue was diluted with saturated aqueous NaHCO₃ solution (40 mL). The resulting solution was extracted with dichloromethane (3×30 mL). The combined organic layers were washed with brine (30 mL), dried over anhydrous sodium sulfate and concentrated under vacuum. The residue was purified by column chromatography using 60% petroleum ether and 40% ethyl acetate as eluents to afford 2-ethyl-8-methyl-3-nitro-8-(trifluoromethyl)-7,8-dihydro-6H-pyrazolo[1,5-a]pyrrolo[2,3-e]pyrimidine (160 mg, 95% yield) as a yellow solid. ¹H NMR (400 MHz, DMSO-d₆) δ: 8.62 (s, 1H), 6.45 (br, 1H), 3.94-3.99 (m, 1H), 3.60-3.69 (m, 1H), 3.09 (q, J=7.6 Hz, 2H), 1.83 (s, 3H), 1.28 (t, J=7.2 Hz, 3H). LC-MS: m/z 316 [M+H]⁺.

### Step 6: N-(5-chloro-6-(2H-1,2,3-triazol-2-yl)pyridin-3-yl)-2-ethyl-8-methyl-3-nitro-8-(trifluoromethyl)-7,8-dihydro-6H-pyrazolo[1,5-a]pyrrolo[2,3-e]pyrimidine-6-carboxamide

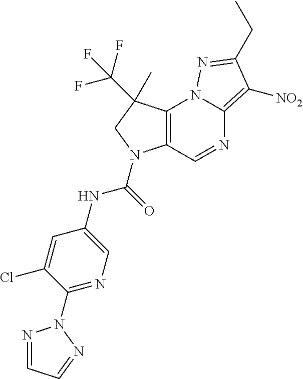 [SMILES: CCc1nn2c3c(cnc2c1[N+](=O)[O-])N(C(=O)Nc1cnc(-n2nccn2)c(Cl)c1)CC3(C)C(F)(F)F]

To a stirred solution of 5-chloro-6-(2H-1,2,3-triazol-2-yl)pyridin-3-amine (Method A1 step 2; 100 mg, 511.1 μmol) in tetrahydrofuran (30 mL) were added triphosgene (91 mg, 307.4 μmol) and TEA (77 mg, 762.3 μmol) at 0° C. The resulting mixture was stirred at 25° C. for 0.5 h and then filtered. The filtrate was added to a solution of 2-ethyl-8-methyl-3-nitro-8-(trifluoromethyl)-7,8-dihydro-6H-pyrazolo[1,5-a]pyrrolo[2,3-e]pyrimidine (160 mg, 511.1 μmol) in tetrahydrofuran (2 mL). To this solution were then added TEA (517 mg, 5.1 mmol) and N,N-dimethylpyridin-4-amine (124 mg, 1.0 mmol). The mixture was stirred at 40° C. for 2 h. The mixture was quenched with water (10 mL), and the resulting solution was extracted with ethyl acetate (3×10 mL). The combined organic layers were washed with brine (20 mL), dried over anhydrous sodium sulfate and concentrated under vacuum. The residue was purified by Prep-HPLC and the collected fractions were concentrated under vacuum to afford N-(5-chloro-6-(2H-1,2,3-triazol-2-yl)pyridin-3-yl)-2-ethyl-8-methyl-3-nitro-8-(trifluoromethyl)-7,8-dihydro-6H-pyrazolo[1,5-a]pyrrolo[2,3-e]pyrimidine-6-carboxamide (180 mg, 65% yield) as a yellow solid. ¹H NMR (400 MHz, DMSO-d₆) δ: 9.78 (s, 1H), 9.60 (s, 1H), 8.74 (d, J=2.4 Hz, 1H), 8.51 (d, J=2.4 Hz, 1H), 8.16 (s, 2H), 4.91 (d, J=11.6 Hz, 1H), 4.34 (d, J=11.6 Hz, 1H), 3.16 (q, J=7.6 Hz, 2H), 2.01 (s, 3H), 1.32 (t, J=7.6 Hz, 3H). LC-MS: m/z 537 [M+H]⁺.

### Step 7: 3-amino-N-(5-chloro-6-(2H-1,2,3-triazol-2-yl)pyridin-3-yl)-2-ethyl-8-methyl-8-(trifluoromethyl)-7,8-dihydro-6H-pyrazolo[1,5-a]pyrrolo[2,3-e]pyrimidine-6-carboxamide

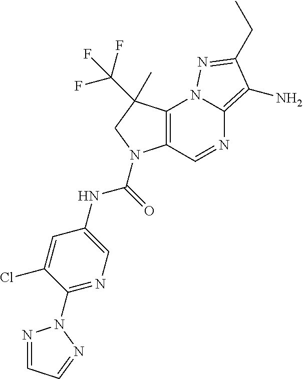 [SMILES: CCc1nn2c3c(cnc2c1N)N(C(=O)Nc1cnc(-n2nccn2)c(Cl)c1)CC3(C)C(F)(F)F]

To a stirred solution of N-(5-chloro-6-(2H-1,2,3-triazol-2-yl)pyridin-3-yl)-2-ethyl-8-methyl-3-nitro-8-(trifluoromethyl)-7,8-dihydro-6H-pyrazolo[1,5-a]pyrrolo[2,3-e]pyrimidine-6-carboxamide (80 mg, 149.0 μmol) in dichloromethane (2 mL) and methanol (2 mL) were added saturated aqueous NH₄Cl solution (2 mL) and Fe (83 mg, 1.5 mmol). The mixture was stirred at 25° C. for 16 h. The reaction mixture was quenched with water (30 mL). The resulting solution was then extracted with ethyl acetate (3×30 mL). The combined organic layers were washed with brine (50 mL) and dried over anhydrous sodium sulfate and concentrated under vacuum. The residue was submitted to Prep-HPLC purification and the collected fractions were lyophilized to give 3-amino-N-(5-chloro-6-(2H-1,2,3-triazol-2-yl)pyridin-3-yl)-2-ethyl-8-methyl-8-(trifluoromethyl)-7,8-dihydro-6H-pyrazolo[1,5-a]pyrrolo[2,3-e]pyrimidine-6-carboxamide (44 mg, 57% yield) as a yellow solid. ¹H NMR (300 MHz, DMSO-d₆) δ: 9.47 (br, 1H), 8.94 (s, 1H), 8.72 (d, J=2.1 Hz, 1H), 8.50 (d, J=2.1 Hz, 1H), 8.15 (s, 2H), 4.74 (d, J=11.4 Hz, 1H), 4.11-4.43 (m, 3H), 2.72 (q, J=7.6 Hz, 2H), 1.95 (s, 3H), 1.21 (t, J=7.6 Hz, 3H). LC-MS: m/z 507 [M+H]⁺.

### Step 8: Separation of enantiomers to obtain (S)-3-amino-N-(5-chloro-6-(2H-1,2,3-triazol-2-yl)pyridin-3-yl)-2-ethyl-8-methyl-8-(trifluoromethyl)-7,8-dihydro-6H-pyrazolo[1,5-a]pyrrolo[2,3-e]pyrimidine-6-carboxamide and (R)-3-amino-N-(5-chloro-6-(2H-1,2,3-triazol-2-yl)pyridin-3-yl)-2-ethyl-8-methyl-8-(trifluoromethyl)-7,8-dihydro-6H-pyrazolo[1,5-a]pyrrolo[2,3-e]pyrimidine-6-carboxamide

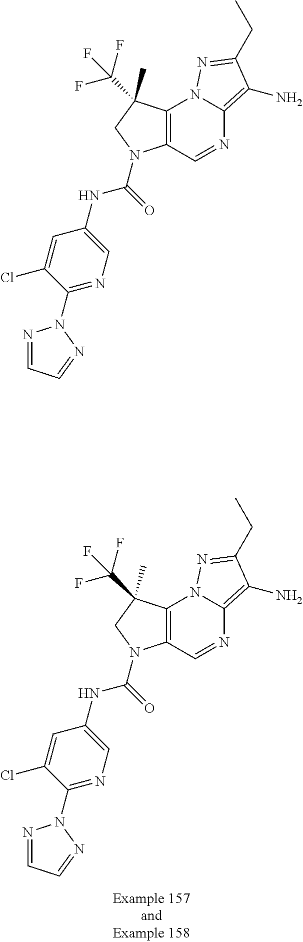 [SMILES: CCc1nn2c3c(cnc2c1N)N(C(=O)Nc1cnc(-n2nccn2)c(Cl)c1)C[C@]3(C)C(F)(F)F ; CCc1nn2c3c(cnc2c1N)N(C(=O)Nc1cnc(-n2nccn2)c(Cl)c1)C[C@@]3(C)C(F)(F)F]

3-amino-N-(5-chloro-6-(2H-1,2,3-triazol-2-yl)pyridin-3-yl)-2-ethyl-8-methyl-8-(trifluoromethyl)-7,8-dihydro-6H-pyrazolo[1,5-a]pyrrolo[2,3-e]pyrimidine-6-carboxamide (38 mg, 74.9 μmol) was submitted to chiral-HPLC: Column: CHIRAL ART Cellulose-SB, 2×25 cm, 5 um; Mobile Phase A: Hex (0.5% 2M NH₃-MeOH)—HPLC, Mobile Phase B: EtOH—HPLC; Flow rate: 20 mL/min; Gradient: 50 B to 50 B in 18 min; 254/220 nm; RT1: 11.603; RT2: 15.848; Injection Volume: 1 ml; Number Of Runs: 9; The first eluting isomer was concentrated and lyophilized to afford Example 157 (10.8 mg, 28% yield) as a white solid. The second eluting isomer was concentrated and lyophilized to afford Example 158 (9.9 mg, 25% yield) as a white solid. Examples 157 and 158 are enantiomers, but their absolute stereochemistry is not yet known.

Example 157: ¹H NMR (300 MHz, DMSO-d₆) δ: 9.47 (br, 1H), 8.94 (s, 1H), 8.72 (d, J=2.1 Hz, 1H), 8.50 (d, J=2.1 Hz, 1H), 8.15 (s, 2H), 4.74 (d, J=11.4 Hz, 1H), 4.11-4.43 (m, 3H), 2.72 (q, J=7.6 Hz, 2H), 1.96 (s, 3H), 1.21 (t, J=7.6 Hz, 3H). LC-MS: m/z 507 [M+H]⁺.

Example 158: ¹H NMR (300 MHz, DMSO-d₆) δ: 9.58 (s, 1H), 8.97 (s, 1H), 8.75 (d, J=2.1 Hz, 1H), 8.52 (d, J=2.1 Hz, 1H), 8.17 (s, 2H), 4.77 (d, J=11.7 Hz, 1H), 4.23-4.38 (m, 3H), 2.74 (q, J=7.6 Hz, 2H), 1.98 (s, 3H), 1.24 (t, J=7.6 Hz, 3H). LC-MS: m/z 507 [M+H]⁺.

### Method I5

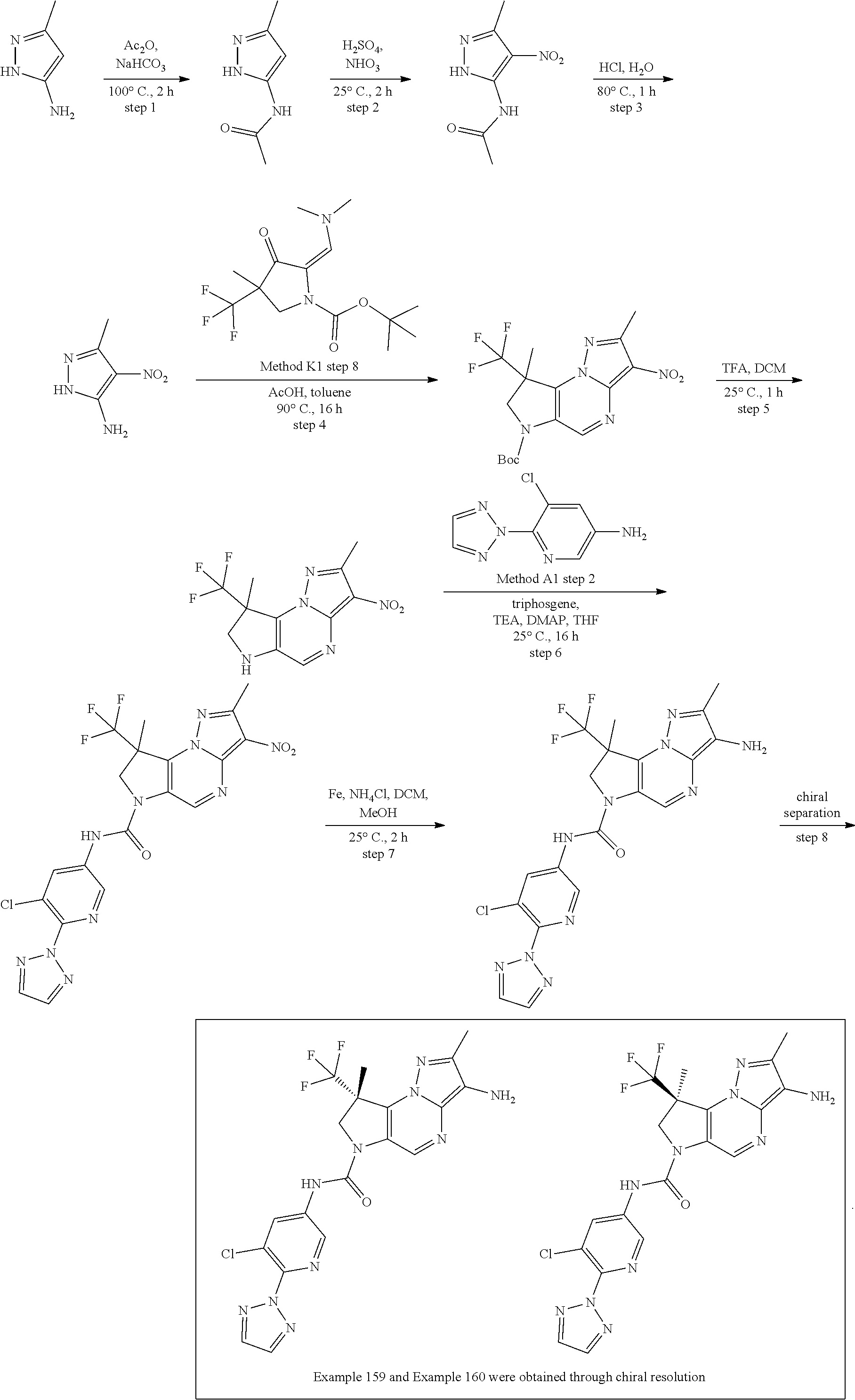

### Example 159 and 160: Single enantiomers obtained from a racemic mixture containing (S)-3-amino-N-(5-chloro-6-(2H-1,2,3-triazol-2-yl)pyridin-3-yl)-2,8-dimethyl-8-(trifluoromethyl)-7,8-dihydro-6H-pyrazolo[1,5-a]pyrrolo[2,3-e]pyrimidine-6-carboxamide and (R)-3-amino-N-(5-chloro-6-(2H-1,2,3-triazol-2-yl)pyridin-3-yl)-2,8-dimethyl-8-(trifluoromethyl)-7,8-dihydro-6H-pyrazolo[1,5-a]pyrrolo[2,3-e]pyrimidine-6-carboxamide

### Step 1: N-(3-methyl-1H-pyrazol-5-yl)acetamide

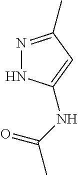

Into a solution of 3-methyl-1H-pyrazol-5-amine (10.00 g, 102.9 mmol) in water (100 mL) were added NaHCO₃ (25.95 g, 308.9 mmol) and acetic anhydride (21.02 g, 205.9 mmol) at 25° C. The reaction mixture was stirred at 100° C. for 2 h, and then the reaction mixture was stirred at 25° C. for 16 h. The solid was collected to afford N-(3-methyl-1H-pyrazol-5-yl)acetamide (7 g, 49% yield) as a white solid. ¹H NMR (400 MHz, DMSO-d₆) δ: 11.89 (br, 1H), 10.17 (br, 1H), 6.21 (s, 1H), 2.15 (s, 3H), 1.94 (s, 3H). LC-MS: m/z 140 [M+H]⁺.

### Step 2: N-(3-methyl-4-nitro-1H-pyrazol-5-yl)acetamide

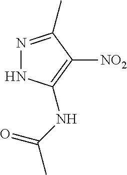

Into a solution of N-(3-methyl-1H-pyrazol-5-yl)acetamide (7 g, 50.3 mmol) in concentrated sulfuric acid (20 mL) was added fuming nitric acid (2.5 mL) at 0° C. The reaction solution was stirred at 25° C. for 2 h. The reaction solution was poured onto ice-water (100 mL). The solid was collected to afford N-(3-methyl-4-nitro-1H-pyrazol-5-yl)acetamide (5 g, 37% yield) as a yellow solid. ¹H NMR (400 MHz, DMSO-d₆) δ: 13.40 (br, 1H), 10.21 (br, 1H), 2.43 (s, 3H), 2.12 (s, 3H). LC-MS: m/z 185 [M+H]⁺.

### Step 3: 3-methyl-4-nitro-1H-pyrazol-5-amine

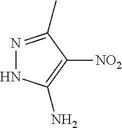

A mixture of N-(3-methyl-4-nitro-1H-pyrazol-5-yl)acetamide (5 g, 27.15 mmol) in water (20 mL) and concentrated HCl (20 mL) were stirred at 80° C. for 1 h. The reaction mixture was cooled to 25° C. The reaction mixture was concentrated. The solid was washed by diethyl ether (50 mL). The solid was collected to afford 3-methyl-4-nitro-1H-pyrazol-5-amine (3 g, 71% yield) as a yellow solid. ¹H NMR (400 MHz, DMSO-d₆) δ: 9.77 (br, 2H), 2.29 (s, 3H). LC-MS: m/z 143 [M+H]⁺.

### Step 4: tert-butyl 2,8-dimethyl-3-nitro-8-(trifluoromethyl)-7,8-dihydro-6H-pyrazolo[1,5-a]pyrrolo[2,3-e]pyrimidine-6-carboxylate

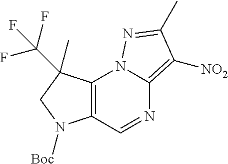 [SMILES: Cc1nn2c3c(cnc2c1[N+](=O)[O-])N(C(=O)OC(C)(C)C)CC3(C)C(F)(F)F]

To a stirred solution of 3-methyl-4-nitro-1H-pyrazol-5-amine (532 mg, 3.7 mmol) in toluene (10 mL) were added tert-butyl (E)-2-((dimethylamino)methylene)-4-methyl-3-oxo-4-(trifluoromethyl)pyrrolidine-1-carboxylate (Method K1 step 8; 1.20 g, 3.7 mmol) and acetic acid (1 mL) at 25° C. The reaction mixture was stirred at 90° C. for 16 h. The reaction solution was cooled to 25° C. The reaction solution was concentrated. The residue was diluted with saturated aqueous NaHCO₃ solution (30 mL). The resulted mixture was extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate as eluents to afford tert-butyl 2,8-dimethyl-3-nitro-8-(trifluoromethyl)-7,8-dihydro-6H-pyrazolo[1,5-a]pyrrolo[2,3-e]pyrimidine-6-carboxylate (380 mg, 25% yield) as a yellow solid. LC-MS: m/z 402 [M+H]⁺.

### Step 5: 2,8-dimethyl-3-nitro-8-(trifluoromethyl)-7,8-dihydro-6H-pyrazolo[1,5-a]pyrrolo[2,3-e]pyrimidine

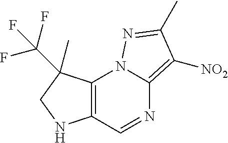 [SMILES: Cc1nn2c3c(cnc2c1[N+](=O)[O-])NCC3(C)C(F)(F)F]

To a stirred solution of tert-butyl 2,8-dimethyl-3-nitro-8-(trifluoromethyl)-7,8-dihydro-6H-pyrazolo[1,5-a]pyrrolo[2,3-e]pyrimidine-6-carboxylate (228 mg, 568.1 μmol) in dichloromethane (10 mL) was added TFA (3 mL) at 25° C. The reaction solution was stirred at 25° C. for 1 h. The pH was adjusted to 8 with saturated aqueous NaHCO₃ solution. The resulted solution was extracted with dichloromethane (3×100 mL). The combined organic layers were dried over anhydrous sodium sulfate and concentrated. The residue was purified by column chromatography on silica gel using 25% petroleum ether and 75% ethyl acetate as eluents to afford 2,8-dimethyl-3-nitro-8-(trifluoromethyl)-7,8-dihydro-6H-pyrazolo[1,5-a]pyrrolo[2,3-e]pyrimidine (114 mg, 63% yield) as a yellow solid. ¹H NMR (400 MHz, DMSO-d₆) δ: 8.62 (s, 1H), 6.45 (s, 1H), 3.97 (dd, J=11.6, 1.6 Hz, 1H), 3.64 (dd, J=11.6, 2.0 Hz, 1H), 2.66 (s, 3H), 1.83 (s, 3H). LC-MS: m/z 302 [M+H]⁺.

### Step 6: N-(5-chloro-6-(2H-1,2,3-triazol-2-yl)pyridin-3-yl)-2,8-dimethyl-3-nitro-8-(trifluoromethyl)-7,8-dihydro-6H-pyrazolo[1,5-a]pyrrolo[2,3-e]pyrimidine-6-carboxamide

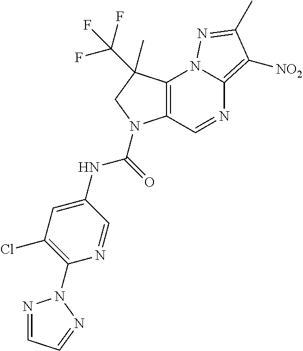 [SMILES: Cc1nn2c3c(cnc2c1[N+](=O)[O-])N(C(=O)Nc1cnc(-n2nccn2)c(Cl)c1)CC3(C)C(F)(F)F]

To a stirred solution of 5-chloro-6-(triazol-2-yl)pyridin-3-amine (Method A1 step 2; 40 mg, 208.1 μmol) in tetrahydrofuran (5 mL) were added triphosgene (34 mg, 113.5 μmol) and TEA (28 mg, 283.8 μmol) at 0° C. The resulting mixture was stirred at 25° C. for 1 h and then filtered. The filtrate was added to a solution of 2,8-dimethyl-3-nitro-8-(trifluoromethyl)-7,8-dihydro-6H-pyrazolo[1,5-a]pyrrolo[2,3-e]pyrimidine (54 mg, 178.8 μmol) in tetrahydrofuran (5 mL). To this solution was then added N,N-dimethylpyridin-4-amine (46 mg, 378.4 μmol). The mixture was stirred at 25° C. for 16 h. The reaction mixture was concentrated under vacuum. The residue was purified by Prep-HPLC and the collected fractions were lyophilized to give N-(5-chloro-6-(2H-1,2,3-triazol-2-yl)pyridin-3-yl)-2,8-dimethyl-3-nitro-8-(trifluoromethyl)-7,8-dihydro-6H-pyrazolo[1,5-a]pyrrolo[2,3-e]pyrimidine-6-carboxamide (52 mg, 48% yield) as a yellow solid. LC-MS: m/z 523 [M+H]⁺.

### Step 7: 3-amino-N-(5-chloro-6-(2H-1,2,3-triazol-2-yl)pyridin-3-yl)-2,8-dimethyl-8-(trifluoromethyl)-7,8-dihydro-6H-pyrazolo[1,5-a]pyrrolo[2,3-e]pyrimidine-6-carboxamide

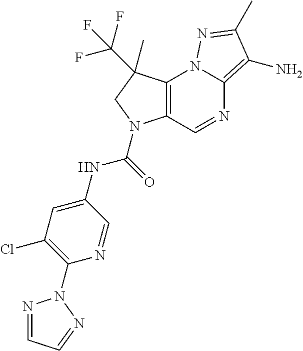 [SMILES: Cc1nn2c3c(cnc2c1N)N(C(=O)Nc1cnc(-n2nccn2)c(Cl)c1)CC3(C)C(F)(F)F]

To a stirred solution of N-(5-chloro-6-(2H-1,2,3-triazol-2-yl)pyridin-3-yl)-2,8-dimethyl-3-nitro-8-(trifluoromethyl)-7,8-dihydro-6H-pyrazolo[1,5-a]pyrrolo[2,3-e]pyrimidine-6-carboxamide (46 mg, 87.98 μmol) in methanol (25 mL) and dichloromethane (25 mL) were added saturated aqueous NH₄Cl solution (25 mL) and Fe (49 mg, 879.8 μmol) under nitrogen. The reaction mixture was stirred at 25° C. for 2 h. The reaction mixture was extracted with dichloromethane (3×30 mL). The combined organic layers were dried over anhydrous sodium sulfate and concentrated. The residue was purified by Prep-HPLC and the collected fractions were lyophilized to give 3-amino-N-(5-chloro-6-(2H-1,2,3-triazol-2-yl)pyridin-3-yl)-2,8-dimethyl-8-(trifluoromethyl)-7,8-dihydro-6H-pyrazolo[1,5-a]pyrrolo[2,3-e]pyrimidine-6-carboxamide (28.6 mg, 66% yield) as a yellow solid. ¹H NMR (300 MHz, DMSO-d₆) δ: 9.58 (br, 1H), 8.98 (s, 1H), 8.74 (d, J=2.4 Hz, 1H), 8.52 (d, J=2.1 Hz, 1H), 8.18 (s, 2H), 4.77 (d, J=11.7 Hz, 1H), 4.30 (br, 2H), 4.25 (d, J=11.7 Hz, 1H), 2.35 (s, 3H), 1.97 (s, 3H). LC-MS: m/z 493 [M+H]⁺.

### Step 8: Separation of enantiomers to obtain (S)-3-amino-N-(5-chloro-6-(2H-1,2,3-triazol-2-yl)pyridin-3-yl)-2,8-dimethyl-8-(trifluoromethyl)-7,8-dihydro-6H-pyrazolo[1,5-a]pyrrolo[2,3-e]pyrimidine-6-carboxamide and (R)-3-amino-N-(5-chloro-6-(2H-1,2,3-triazol-2-yl)pyridin-3-yl)-2,8-dimethyl-8-(trifluoromethyl)-7,8-dihydro-6H-pyrazolo[1,5-a]pyrrolo[2,3-e]pyrimidine-6-carboxamide

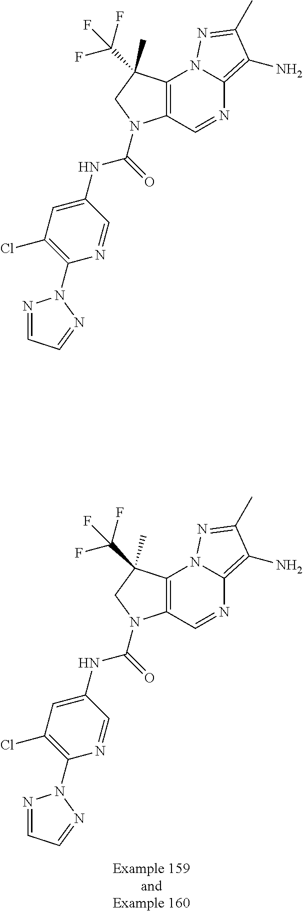 [SMILES: Cc1nn2c3c(cnc2c1N)N(C(=O)Nc1cnc(-n2nccn2)c(Cl)c1)C[C@]3(C)C(F)(F)F ; Cc1nn2c3c(cnc2c1N)N(C(=O)Nc1cnc(-n2nccn2)c(Cl)c1)C[C@@]3(C)C(F)(F)F]

25 mg of 3-amino-N-(5-chloro-6-(2H-1,2,3-triazol-2-yl)pyridin-3-yl)-2,8-dimethyl-8-(trifluoromethyl)-7,8-dihydro-6H-pyrazolo[1,5-a]pyrrolo[2,3-e]pyrimidine-6-carboxamide was submitted to chiral-HPLC: CHIRAL ART Cellulose-SB, 2×25 cm, 5 um; Mobile Phase A: Hex(0.5% 2M NH₃-MeOH)—HPLC, Mobile Phase B: EtOH—HPLC; Flow rate: 20 mL/min; Gradient: 50 B to 50 B in 21 min; 220/254 nm; RT1: 14.363; RT2: 19.752; Injection Volume: 0.7 ml; Number Of Runs: 10. The first eluting isomer was concentrated and lyophilized to afford Example 159 (3.1 mg, 12% yield) as a yellow solid. The second eluting isomer was concentrated and lyophilized to afford Example 160 (3.5 mg, 14% yield) as a yellow solid. Examples 159 and 160 are enantiomers, but their absolute stereochemistry is not yet known.

Example 159: ¹H NMR (300 MHz, DMSO-d₆) δ: 9.57 (br, 1H), 8.96 (s, 1H), 8.74 (d, J=2.3 Hz, 1H), 8.51 (d, J=2.3 Hz, 1H), 8.17 (s, 2H), 4.76 (d, J=11.5 Hz, 1H), 4.30 (br, 2H), 4.24 (d, J=11.5 Hz, 1H), 2.34 (s, 3H), 1.96 (s, 3H). LC-MS: m/z 493 [M+H]⁺.

Example 160: ¹H NMR (400 MHz, DMSO-d₆) δ: 9.57 (br, 1H), 8.96 (s, 1H), 8.73 (d, J=2.3 Hz, 1H), 8.51 (d, J=2.3 Hz, 1H), 8.17 (s, 2H), 4.76 (d, J=11.5 Hz, 1H), 4.30 (br, 2H), 4.24 (d, J=11.5 Hz, 1H), 2.34 (s, 3H), 1.96 (s, 3H). LC-MS: m/z 493 [M+H]⁺.

### Method J5

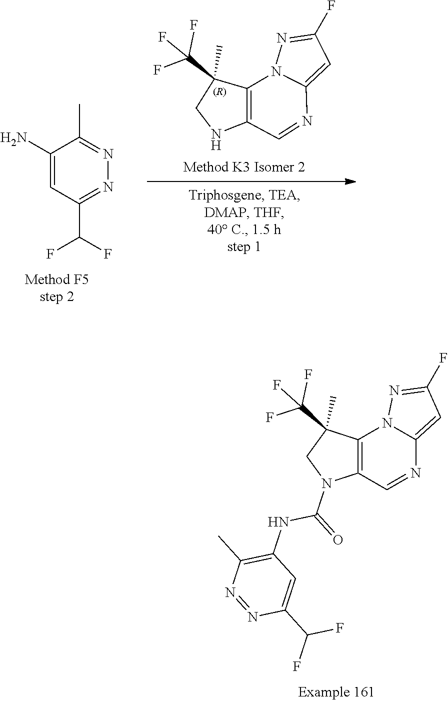

### Example 161: (R)—N-(6-(difluoromethyl)-3-methylpyridazin-4-yl)-2-fluoro-8-methyl-8-(trifluoromethyl)-7,8-dihydro-6H-pyrazolo[1,5-a]pyrrolo[2,3-e]pyrimidine-6-carboxamide

### Step 1: (R)—N-(6-(difluoromethyl)-3-methylpyridazin-4-yl)-2-fluoro-8-methyl-8-(trifluoromethyl)-7,8-dihydro-6H-pyrazolo[1,5-a]pyrrolo[2,3-e]pyrimidine-6-carboxamide

To a stirred solution of 6-(difluoromethyl)-3-methylpyridazin-4-amine (Method F5 step 2; 40 mg, 251.4 μmol) in tetrahydrofuran (8 mL) were added triphosgene (45 mg, 150.8 μmol) and TEA (38 mg, 377 μmol). The resulting mixture was stirred at 40° C. for 0.5 h and then filtered. The filtrate was added to a solution of (R)-2-fluoro-8-methyl-8-(trifluoromethyl)-7,8-dihydro-6H-pyrazolo[1,5-a]pyrrolo[2,3-e]pyrimidine (Method K3 isomer 2; 52 mg, 200 μmol) in tetrahydrofuran (2 mL). To this solution was then added TEA (255 mg, 2.5 mmol) and N,N-dimethylpyridin-4-amine (61 mg, 502.7 μmol). The mixture was stirred at 40° C. for 1.5 h. The mixture was concentrated under vacuum. The residue was purified by Prep-TLC using 96% dichloromethane and 4% methanol as eluents to afford 90 mg of the crude product which was purified by Prep-HPLC purification and the collected fractions were lyophilized to give (R)—N-(6-(difluoromethyl)-3-methylpyridazin-4-yl)-2-fluoro-8-methyl-8-(trifluoromethyl)-7,8-dihydro-6H-pyrazolo[1,5-a]pyrrolo[2,3-e]pyrimidine-6-carboxamide (29.2 mg, 33% yield) as a white solid. The enantiomer of Example 161 can be prepared analogously using Method K3 isomer 1.

Example 161: ¹H NMR (300 MHz, DMSO-d₆) δ: 9.31 (s, 1H), 8.87 (br, 1H), 8.25 (s, 1H), 7.30 (t, J=54.3 Hz, 1H), 6.71 (d, J=5.1 Hz, 1H), 4.96 (d, J=11.4 Hz, 1H), 4.38 (d, J=11.4 Hz, 1H), 2.78 (s, 3H), 1.98 (s, 3H). LC-MS: m/z 446 [M+H]⁺.

### Method K5

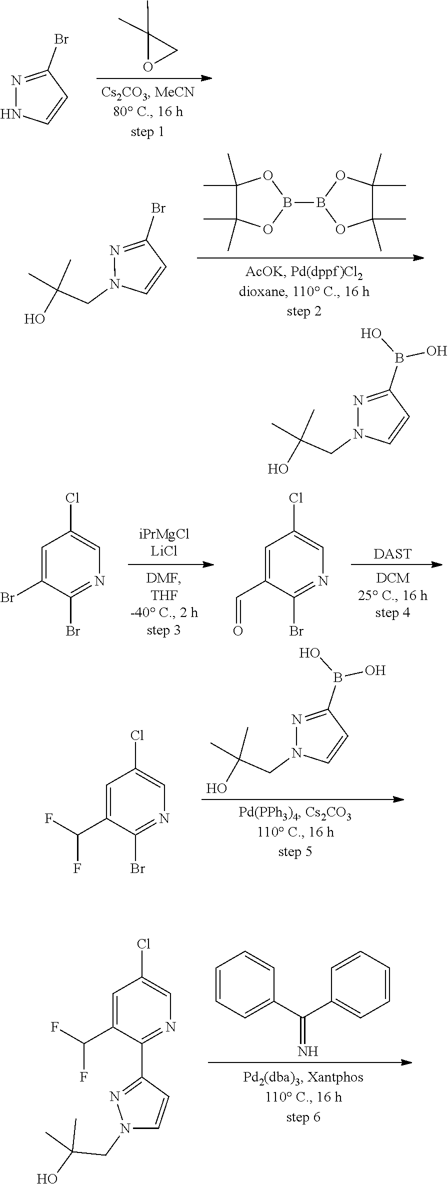

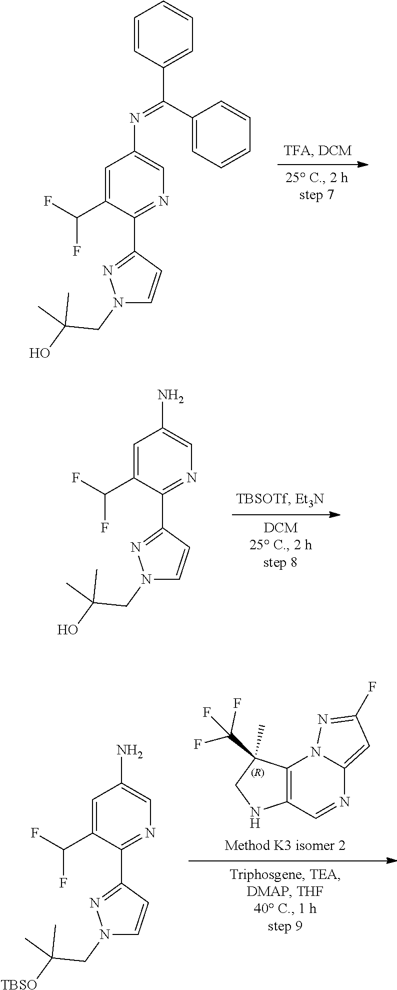

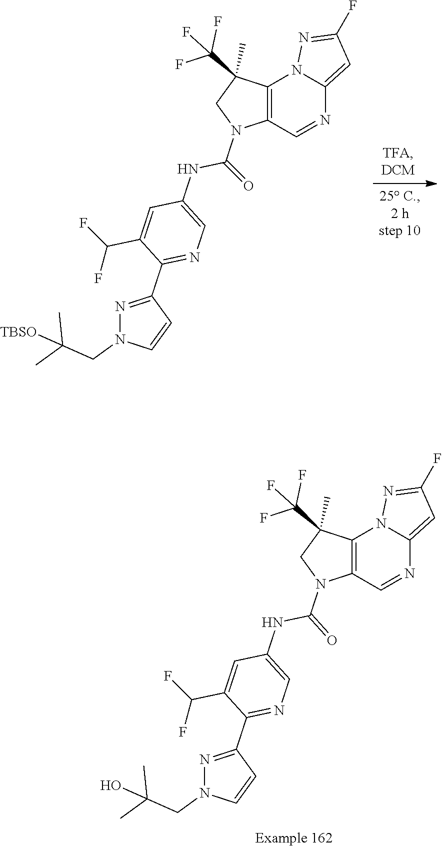 [SMILES: CC(C)(Cn1ccc(-c2ncc(NC(=O)N3C[C@@](C)(C(F)(F)F)c4c3cnc3cc(F)nn43)cc2C(F)F)n1)O[Si](C)(C)C(C)(C)C ; CC(C)(O)Cn1ccc(-c2ncc(NC(=O)N3C[C@@](C)(C(F)(F)F)c4c3cnc3cc(F)nn43)cc2C(F)F)n1]

### Example 162: (R)—N-(5-(difluoromethyl)-6-(1-(2-hydroxy-2-methylpropyl)-1H-pyrazol-3-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

### Step 1: 1-(3-bromo-1H-pyrazol-1-yl)-2-methylpropan-2-ol

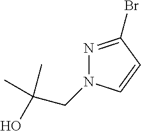

To a stirred mixture of 3-bromo-1H-pyrazole (10.0 g, 68.0 mmol) in acetonitrile (100 mL) were added cesium carbonate (44.3 g, 136.1 mmol) and 2,2-dimethyloxirane (24.5 g, 340.2 mmol). The mixture was stirred at 80° C. for 16 h. The resulting mixture was concentrated under vacuum. The residue was diluted with water (100 mL). The resulting solution was extracted with ethyl acetate (3×100 mL). The combined organic layers were dried over anhydrous sodium sulfate and concentrated under vacuum. The residue was purified by column chromatography on silica gel using 95% petroleum ether and 5% ethyl acetate as eluents to afford 1-(3-bromo-1H-pyrazol-1-yl)-2-methylpropan-2-ol (18.5 g, 37% yield) as a yellow oil. LC-MS: m/z 219 [M+H]⁺.

### Step 2: (1-(2-hydroxy-2-methylpropyl)-1H-pyrazol-3-yl)boronic acid

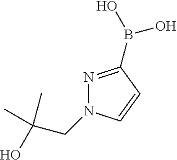

To a stirred mixture of 1-(3-bromo-1H-pyrazol-1-yl)-2-methylpropan-2-ol (8.0 g, 36.5 mmol) in dioxane (80 mL) were added 4,4,4′,4′,5,5,5′,5′-octamethyl-2,2′-bi(1,3,2-dioxaborolane) (11.1 g, 43.8 mmol), potassium acetate (10.8 g, 109.6 mmol) and Pd(dppf)Cl₂ (5.3 g, 7.3 mmol) under nitrogen atmosphere. The reaction mixture was stirred at 110° C. for 16 h. The resulting mixture was concentrated under vacuum. The residue was purified by Prep-HPLC and the collected fractions were concentrated to afford (1-(2-hydroxy-2-methylpropyl)-1H-pyrazol-3-yl)boronic acid (8.5 g, 38% yield) as a colorless oil. LC-MS: m/z 185 [M+H]⁺.

### Step 3: 2-bromo-5-chloronicotinaldehyde

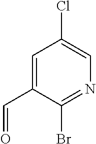

To a stirred solution of 2,3-dibromo-5-chloropyridine (10.0 g, 36.9 mmol) in tetrahydrofuran (50 mL) was added isopropylmagnesium(II)lithium chloride (31.2 mL, 40.6 mmol, 1.3 M in tetrahydrofuran) dropwise at −40° C. under nitrogen atmosphere. The reaction mixture was stirred at −40° C. for 1 h. N,N-dimethylformamide (20 mL) was added dropwise at −40° C. The reaction mixture was stirred at 25° C. for 1 h. The reaction mixture was quenched with HCl (50 mL, 1M). The resulting mixture was extracted with ethyl acetate (3×100 mL). The combined organic layers were dried over anhydrous sodium sulfate and concentrated under vacuum. The residue was purified by column chromatography on silica gel using 90% petroleum ether and 10% ethyl acetate as eluents to afford 2-bromo-5-chloronicotinaldehyde (5.4 g, 66% yield) as a yellow solid. LC-MS: m/z 220 [M+H]⁺.

### Step 4: 2-bromo-5-chloro-3-(difluoromethyl)pyridine

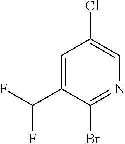 [SMILES: FC(F)c1cc(Cl)cnc1Br]

To a stirred solution of 2-bromo-5-chloronicotinaldehyde (5.4 g, 25.0 mmol) in dichloromethane (100 mL) was added DAST (12.1 g, 74.9 mmol) dropwise at 0° C. The reaction mixture was stirred at 25° C. for 16 h. The reaction mixture was quenched with water (100 mL). The resulting solution was extracted with dichloromethane (3×100 mL). The combined organic layers were dried over anhydrous sodium sulfate and concentrated under vacuum. The residue was purified by column chromatography on silica gel using 90% petroleum ether and 10% ethyl acetate as eluents to afford 2-bromo-5-chloro-3-(difluoromethyl)pyridine (1.3 g, 21% yield) as a yellow oil. ¹H NMR (300 MHz, DMSO-d₆) δ: 8.68-8.70 (m, 1H), 8.26 (d, J=2.4 Hz, 1H), 7.15 (t, J=53.4 Hz, 1H). LC-MS: m/z 242 [M+H]⁺.

### Step 5: 1-(3-(5-chloro-3-(difluoromethyl)pyridin-2-yl)-1H-pyrazol-1-yl)-2-methylpropan-2-ol

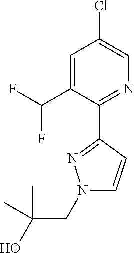 [SMILES: CC(C)(O)Cn1ccc(-c2ncc(Cl)cc2C(F)F)n1]

To a stirred mixture of 2-bromo-5-chloro-3-(difluoromethyl)pyridine (1.2 g, 5.0 mmol) in dioxane (30 mL) and water (20 mL) were added (1-(2-hydroxy-2-methylpropyl)-1H-pyrazol-3-yl)boronic acid (9.1 g, 49.5 mmol), cesium carbonate (4.0 g, 12.4 mmol) and Pd(PPh₃)₄ (362.2 mg, 495.0 μmol) under nitrogen atmosphere. The reaction mixture was stirred at 110° C. for 16 h. The resulting mixture was concentrated under vacuum. The residue was purified by column chromatography on silica gel using 50% petroleum ether and 50% ethyl acetate as eluents to afford 1-(3-(5-chloro-3-(difluoromethyl)pyridin-2-yl)-1H-pyrazol-1-yl)-2-methylpropan-2-ol (1.8 g, 47% yield) as a colorless oil. ¹H NMR (400 MHz, DMSO-d₆) δ: 8.82-8.83 (m, 1H), 8.22 (d, J=2.4 Hz, 1H), 7.94 (t, J=54.8 Hz, 1H), 7.81 (d, J=2.4 Hz, 1H), 6.89 (d, J=2.4 Hz, 1H), 4.75 (s, 1H), 4.12 (s, 2H), 1.12 (s, 6H). LC-MS: m/z 302 [M+H]⁺.

### Step 6: 1-(3-(3-(difluoromethyl)-5-((diphenylmethylene)amino)pyridin-2-yl)-1H-pyrazol-1-yl)-2-methylpropan-2-ol

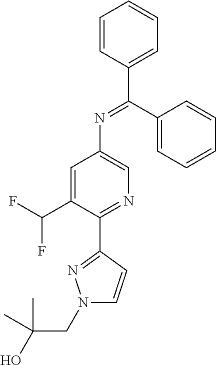 [SMILES: CC(C)(O)Cn1ccc(-c2ncc(N=C(c3ccccc3)c3ccccc3)cc2C(F)F)n1]

To a stirred mixture of 1-(3-(5-chloro-3-(difluoromethyl)pyridin-2-yl)-1H-pyrazol-1-yl)-2-methylpropan-2-ol (1.8 g, 2.3 mmol) in dioxane (20 mL) were added diphenylmethanimine (846 mg, 4.7 mmol), Pd₂(dba)₃ (427 mg, 466.7 μmol) and XantPhos (270 mg, 466.7 μmol) under nitrogen atmosphere. The reaction mixture was stirred at 110° C. for 16 h. The reaction mixture was concentrated under reduced pressure. The residue was purified by column chromatography on silica gel using 90% dichloromethane and 10% methanol as eluents to afford 1-(3-(3-(difluoromethyl)-5-((diphenylmethylene)amino)pyridin-2-yl)-1H-pyrazol-1-yl)-2-methylpropan-2-ol (2.0 g, 92% yield) as a yellow oil. LC-MS: m/z 447 [M+H]⁺.

### Step 7: 1-(3-(5-amino-3-(difluoromethyl)pyridin-2-yl)-1H-pyrazol-1-yl)-2-methylpropan-2-ol

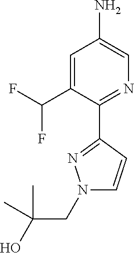 [SMILES: CC(C)(O)Cn1ccc(-c2ncc(N)cc2C(F)F)n1]

To a stirred mixture of 1-(3-(3-(difluoromethyl)-5-((diphenylmethylene)amino)pyridin-2-yl)-1H-pyrazol-1-yl)-2-methylpropan-2-ol (2.0 g, 2.2 mmol) in dichloromethane (15 mL) were added TFA (5 mL). The reaction mixture was stirred at 25° C. for 2 h. The mixture was concentrated under vacuum. The residue was diluted with saturated aqueous NaHCO₃ solution (50 mL). The resulting solution was extracted with ethyl acetate (3×50 mL). The combined organic layers were dried over anhydrous sodium sulfate and concentrated under vacuum. The residue was purified by column chromatography on silica gel using 80% dichloromethane and 20% methanol as eluents to afford 1-(3-(5-amino-3-(difluoromethyl)pyridin-2-yl)-1H-pyrazol-1-yl)-2-methylpropan-2-ol (470 mg, 69% yield) as a yellow oil. LC-MS: m/z 283 [M+H]⁺.

### Step 8: 6-(1-(2-((tert-butyldimethylsilyl)oxy)-2-methylpropyl)-1H-pyrazol-3-yl)-5-(difluoromethyl)pyridin-3-amine

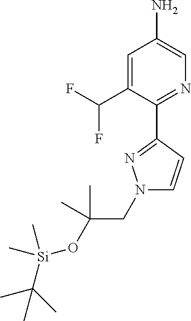 [SMILES: CC(C)(Cn1ccc(-c2ncc(N)cc2C(F)F)n1)O[Si](C)(C)C(C)(C)C]

To a stirred mixture of 1-(3-(5-amino-3-(difluoromethyl)pyridin-2-yl)-1H-pyrazol-1-yl)-2-methylpropan-2-ol (500 mg, 1.8 mmol) in dichloromethane (5 mL) were added TEA (538 mg, 5.3 mmol) and tert-butyldimethylsilyl trifluoromethanesulfonate (936 mg, 3.5 mmol). The reaction mixture was stirred at 25° C. for 2 h. The reaction mixture was concentrated under reduced pressure. The residue was diluted with water (10 mL). The resulting solution was extracted with ethyl acetate (2×10 mL). The combined organic layers were dried over anhydrous sodium sulfate and concentrated under vacuum. The residue was purified by column chromatography on silica gel using 40% petroleum ether and 60% ethyl acetate as eluents to afford 6-(1-(2-((tert-butyldimethylsilyl)oxy)-2-methylpropyl)-1H-pyrazol-3-yl)-5-(difluoromethyl)pyridin-3-amine (330 mg, 42% yield) as a yellow solid. LC-MS: m/z 397 [M+H]⁺.

### Step 9: (R)—N-(6-(1-(2-((tert-butyldimethylsilyl)oxy)-2-methylpropyl)-1H-pyrazol-3-yl)-5-(difluoromethyl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

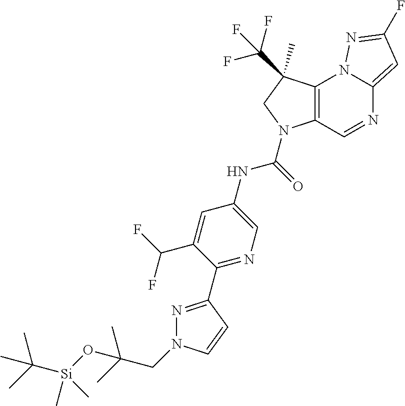 [SMILES: CC(C)(Cn1ccc(-c2ncc(NC(=O)N3C[C@@](C)(C(F)(F)F)c4c3cnc3cc(F)nn43)cc2C(F)F)n1)O[Si](C)(C)C(C)(C)C]

To a stirred solution of 6-(1-(2-((tert-butyldimethylsilyl)oxy)-2-methylpropyl)-1H-pyrazol-3-yl)-5-(difluoromethyl)pyridin-3-amine (70 mg, 176.5 μmol) in tetrahydrofuran (1 mL) were added triphosgene (21 mg, 70.6 μmol) and TEA (17.9 mg, 176.5 μmol). The resulting mixture was stirred at 25° C. for 1 h and then filtered. The filtrate was added to a solution of (R)-2-fluoro-8-methyl-8-(trifluoromethyl)-7,8-dihydro-6H-pyrazolo[1,5-a]pyrrolo[2,3-e]pyrimidine (Method K3 isomer 2; 31 mg, 117.7 μmol) in tetrahydrofuran (1 mL). To this solution were added TEA (119 mg, 1.2 mmol) and N,N-dimethylpyridin-4-amine (22 mg, 176.5 μmol). The mixture was stirred at 40° C. for 1 h. The reaction mixture was quenched with water (10 mL). The resulting solution was extracted with ethyl acetate (3×10 mL). The combined organic layers were dried over anhydrous sodium sulfate and concentrated under vacuum. The residue was purified by column chromatography on silica gel using 80% petroleum ether and 20% ethyl acetate as eluents to afford (R)—N-(6-(1-(2-((tert-butyldimethylsilyl)oxy)-2-methylpropyl)-1H-pyrazol-3-yl)-5-(difluoromethyl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide (44 mg, 21% yield) as a yellow solid. LC-MS: m/z 683 [M+H]⁺.

### Step 10: (R)—N-(5-(difluoromethyl)-6-(1-(2-hydroxy-2-methylpropyl)-1H-pyrazol-3-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

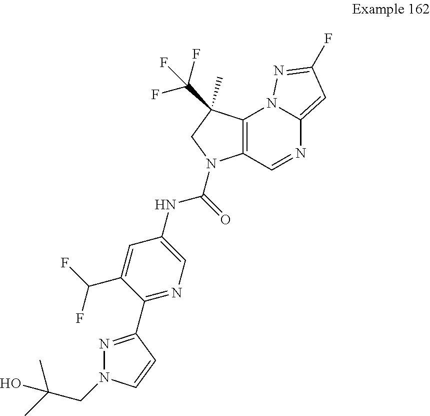 [SMILES: CC(C)(O)Cn1ccc(-c2ncc(NC(=O)N3C[C@@](C)(C(F)(F)F)c4c3cnc3cc(F)nn43)cc2C(F)F)n1]

To a stirred solution of (R)—N-(6-(1-(2-((tert-butyldimethylsilyl)oxy)-2-methylpropyl)-1H-pyrazol-3-yl)-5-(difluoromethyl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide (44 mg, 64.5 μmol) in dichloromethane (2 mL) were added 2,2,2-trifluoroacetic acid (2 mL). The mixture was stirred for 2 h at 25° C. The resulting mixture was concentrated under reduced pressure. The residue was purified by Prep-HPLC and the collected fractions were lyophilized to afford (R)—N-(5-(difluoromethyl)-6-(1-(2-hydroxy-2-methylpropyl)-1H-pyrazol-3-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide (12 mg, 33% yield) as a white solid. The corresponding enantiomers of Example 162 can be prepared analogously using Method K3 isomer 1.

Example 162: ¹H NMR (400 MHz, DMSO-d₆) δ: 9.36 (s, 1H), 8.95 (d, J=2.4 Hz, 1H), 8.41 (d, J=2.4 Hz, 1H), 7.97 (t, J=55.2 Hz, 1H), 7.78 (d, J=2.4 Hz, 1H), 6.84 (d, J=2.4 Hz, 1H), 6.67 (d, J=4.8 Hz, 1H), 4.84 (d, J=11.6 Hz, 1H), 4.73 (s, 1H), 4.29 (d, J=11.6 Hz, 1H), 4.11 (s, 2H), 1.97 (s, 3H), 1.12 (s, 6H). LC-MS: m/z 569 [M+H]⁺.

### Method L5

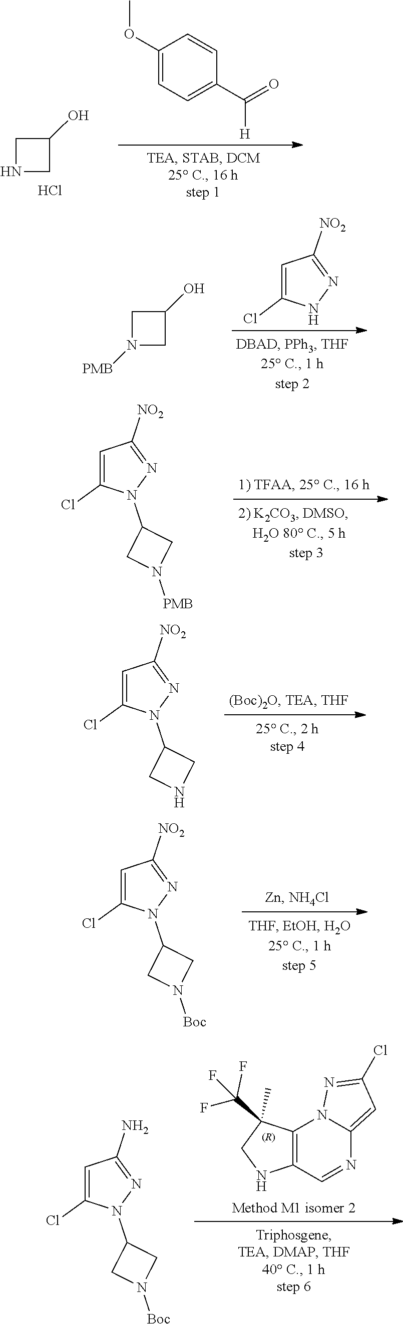

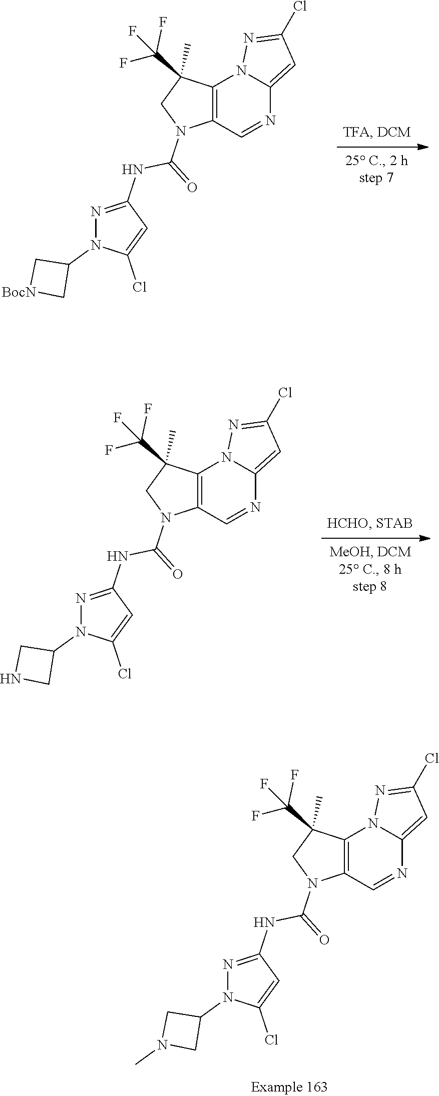 [SMILES: CC(C)(C)OC(=O)N1CC(n2nc(NC(=O)N3C[C@@](C)(C(F)(F)F)c4c3cnc3cc(Cl)nn43)cc2Cl)C1 ; C[C@@]1(C(F)(F)F)CN(C(=O)Nc2cc(Cl)n(C3CNC3)n2)c2cnc3cc(Cl)nn3c21 ; CN1CC(n2nc(NC(=O)N3C[C@@](C)(C(F)(F)F)c4c3cnc3cc(Cl)nn43)cc2Cl)C1]

### Example 163: (R)-2-chloro-N-(5-chloro-1-(1-methylazetidin-3-yl)-1H-pyrazol-3-yl)-8-methyl-8-(trifluoromethyl)-7,8-dihydro-6H-pyrazolo[1,5-a]pyrrolo[2,3-e]pyrimidine-6-carboxamide

### Step 1: 1-(4-methoxybenzyl)azetidin-3-ol

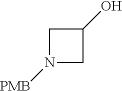

To a mixture of 4-methoxybenzaldehyde (1.00 g, 7.3 mmol) in dichloromethane (10 mL) were added azetidin-3-ol hydrochloride (933 mg, 8.5 mmol) and TEA (862 mg, 8.5 mmol). The mixture was stirred at 25° C. for 1 h, and then sodium triacetoxyhydroborate (3.11 g, 14.7 mmol) was added in portions. The reaction mixture was stirred at 25° C. for 16 h. The reaction mixture was quenched by water (100 mL), and the resulting solution was extracted with dichloromethane (3×100 mL). The combined organic layers were washed with brine (100 mL), dried over sodium sulfate and concentrated under vacuum. The residue was purified by column chromatography on silica gel using 10% petroleum ether and 90% ethyl acetate (1% TEA) as eluents to afford 1-(4-methoxybenzyl)azetidin-3-ol (886 mg, 62% yield) as a colorless oil. ¹H NMR (400 MHz, DMSO-d₆) δ: 7.10-7.16 (m, 2H), 6.80-6.85 (m, 2H), 5.23 (d, J=6.4 Hz, 1H), 3.90-4.20 (m, 1H), 3.70 (s, 3H), 3.37-3.45 (m, 4H), 2.65-2.71 (m, 2H). LC-MS: m/z 194 [M+H]⁺.

### Step 2: 5-chloro-1-(1-(4-methoxybenzyl)azetidin-3-yl)-3-nitro-1H-pyrazole

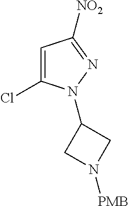

To a stirred solution of 5-chloro-3-nitro-1H-pyrazole (1.0 g, 6.8 mmol) in tetrahydrofuran (100 mL) was added 1-(4-methoxybenzyl)azetidin-3-ol (2.0 g, 10.1 mmol), dibenzyl azodicarboxylate (3.1 g, 13.5 mmol) and triphenylphosphine (3.6 g, 13.6 mmol). The mixture was stirred at 25° C. for 1 h. The mixture was concentrated under vacuum. The residue was purified by column chromatography on silica gel using 50% petroleum ether and 50% ethyl acetate as eluents to afford 5-chloro-1-(1-(4-methoxybenzyl)azetidin-3-yl)-3-nitro-1H-pyrazole (1 g, 60% yield) as a white solid. LC-MS: m/z 323 [M+H]⁺.

### Step 3: 1-(azetidin-3-yl)-5-chloro-3-nitro-1H-pyrazole

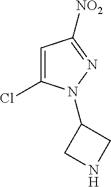 [SMILES: O=[N+]([O-])c1cc(Cl)n(C2CNC2)n1]

A mixture of 5-chloro-1-(1-(4-methoxybenzyl)azetidin-3-yl)-3-nitro-1H-pyrazole (1.0 g, 3.1 mmol) in trifluoroacetic anhydride (20 mL) was stirred at 25° C. for 16 h. The mixture was concentrated, and the residue was diluted in dimethyl sulfoxide (8 mL) and water (4 mL). To this mixture was added K₂CO₃ (1.71 g, 12.4 mmol). The mixture was stirred at 80° C. for 5 h. After cooled to 25° C., the reaction mixture was quenched with water (50 mL). The resulting solution was extracted with ethyl acetate (3×50 mL). The combined organic layers were dried over anhydrous sodium sulfate and concentrated under vacuum to afford 1-(azetidin-3-yl)-5-chloro-3-nitro-1H-pyrazole (620 mg, crude) as a yellow solid, which was used directly without further purification. LC-MS: m/z 203 [M+H]⁺.

### Step 4: tert-butyl 3-(5-chloro-3-nitro-1H-pyrazol-1-yl)azetidine-1-carboxylate

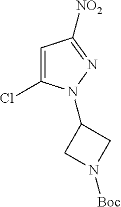 [SMILES: CC(C)(C)OC(=O)N1CC(n2nc([N+](=O)[O-])cc2Cl)C1]

To a stirred solution of 1-(azetidin-3-yl)-5-chloro-3-nitro-1H-pyrazole (200 mg, 987.1 μmol) in tetrahydrofuran (10 mL) was added (Boc)₂O (323 mg, 1.5 mmol) and TEA (300 mg, 3.0 mmol). The mixture was stirred at 25° C. for 2 h. The reaction mixture was concentrated under vacuum. The residue was purified by column chromatography on silica gel using 85% petroleum ether and 15% ethyl acetate as eluents to afford tert-butyl 3-(5-chloro-3-nitro-1H-pyrazol-1-yl)azetidine-1-carboxylate (120 mg, 40% yield) as a white solid. ¹H NMR (300 MHz, DMSO-d₆) δ: 7.44 (s, 1H), 5.39-5.59 (m, 1H), 4.36-4.38 (m, 2H), 4.10-4.23 (m, 2H), 1.42 (s, 9H). LC-MS: m/z 303 [M+H]⁺.

### Step 5: tert-butyl 3-(3-amino-5-chloro-1H-pyrazol-1-yl)azetidine-1-carboxylate

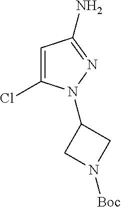 [SMILES: CC(C)(C)OC(=O)N1CC(n2nc(N)cc2Cl)C1]

To a stirred mixture of tert-butyl 3-(5-chloro-3-nitro-1H-pyrazol-1-yl)azetidine-1-carboxylate (150 mg, 495.5 μmol) in tetrahydrofuran (4 mL) were added ethanol (1 mL), water (1 mL), NH₄Cl (398 mg, 7.4 mmol) and Zn (324 mg, 5.0 mmol). The reaction mixture was stirred at 25° C. for 1 h. The solid was filtered out. The filtrate was concentrated. The residue was purified by column chromatography on silica gel using 50% petroleum ether and 50% ethyl acetate as eluents to afford tert-butyl 3-(3-amino-5-chloro-1H-pyrazol-1-yl)azetidine-1-carboxylate (100 mg, 74% yield) as a colorless oil. LC-MS: m/z 273 [M+H]⁺.

### Step 6: tert-butyl (R)-3-(5-chloro-3-(2-chloro-8-methyl-8-(trifluoromethyl)-7,8-dihydro-6H-pyrazolo[1,5-a]pyrrolo[2,3-e]pyrimidine-6-carboxamido)-1H-pyrazol-1-yl)azetidine-1-carboxylate

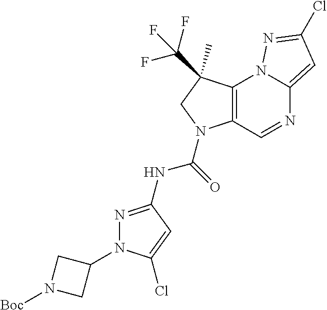 [SMILES: CC(C)(C)OC(=O)N1CC(n2nc(NC(=O)N3C[C@@](C)(C(F)(F)F)c4c3cnc3cc(Cl)nn43)cc2Cl)C1]

To a mixture of Method M1 isomer 2 (40 mg, 144.6 μmol) in tetrahydrofuran (2 mL) was added triphosgene (26 mg, 86.8 μmol) and TEA (22 mg, 216.9 μmol). The mixture was stirred at 25° C. for 0.5 h and then filtered. The filtrate was added to a solution of tert-butyl 3-(3-amino-5-chloro-pyrazol-1-yl)azetidine-1-carboxylate (79 mg, 289.2 μmol) in tetrahydrofuran (2 mL). To this solution were then added TEA (146 mg, 1.5 mmol) and N,N-dimethylpyridin-4-amine (35 mg, 289.1 μmol). The mixture was stirred at 40° C. for 1 h. The reaction mixture was concentrated. The residue was purified by column chromatography on silica gel using 90% dichloromethane and 10% methanol as eluents to afford tert-butyl (R)-3-(5-chloro-3-(2-chloro-8-methyl-8-(trifluoromethyl)-7,8-dihydro-6H-pyrazolo[1,5-a]pyrrolo[2,3-e]pyrimidine-6-carboxamido)-1H-pyrazol-1-yl)azetidine-1-carboxylate (40 mg, 48% yield) as a yellow solid. LC-MS: m/z 575 [M+H]⁺.

### Step 7: (R)—N-(1-(azetidin-3-yl)-5-chloro-1H-pyrazol-3-yl)-2-chloro-8-methyl-8-(trifluoromethyl)-7,8-dihydro-6H-pyrazolo[1,5-a]pyrrolo[2,3-e]pyrimidine-6-carboxamide

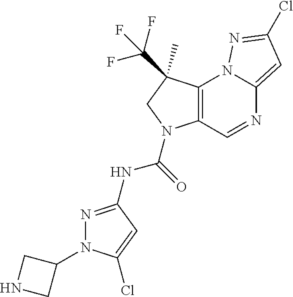 [SMILES: C[C@@]1(C(F)(F)F)CN(C(=O)Nc2cc(Cl)n(C3CNC3)n2)c2cnc3cc(Cl)nn3c21]

To a stirred solution of tert-butyl (R)-3-(5-chloro-3-(2-chloro-8-methyl-8-(trifluoromethyl)-7,8-dihydro-6H-pyrazolo[1,5-a]pyrrolo[2,3-e]pyrimidine-6-carboxamido)-1H-pyrazol-1-yl)azetidine-1-carboxylate (40 mg, 69.5 μmol) in dichloromethane (4 mL) was added TFA (2 mL). The reaction mixture was stirred at 25° C. for 2 h. The reaction mixture was concentrated to afford (R)—N-(1-(azetidin-3-yl)-5-chloro-1H-pyrazol-3-yl)-2-chloro-8-methyl-8-(trifluoromethyl)-7,8-dihydro-6H-pyrazolo[1,5-a]pyrrolo[2,3-e]pyrimidine-6-carboxamide (40 mg, crude) as a yellow solid, which was used directly without further purification. LC-MS: m/z 475 [M+H]⁺.

### Step 8: (R)-2-chloro-N-(5-chloro-1-(1-methylazetidin-3-yl)-1H-pyrazol-3-yl)-8-methyl-8-(trifluoromethyl)-7,8-dihydro-6H-pyrazolo[1,5-a]pyrrolo[2,3-e]pyrimidine-6-carboxamide

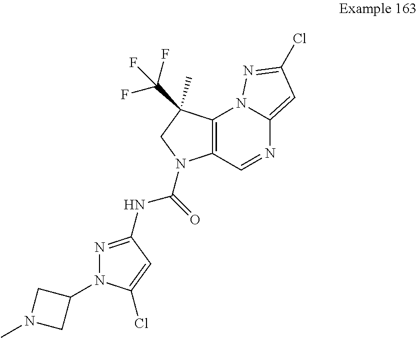 [SMILES: CN1CC(n2nc(NC(=O)N3C[C@@](C)(C(F)(F)F)c4c3cnc3cc(Cl)nn43)cc2Cl)C1]

To a stirred mixture of (R)—N-(1-(azetidin-3-yl)-5-chloro-1H-pyrazol-3-yl)-2-chloro-8-methyl-8-(trifluoromethyl)-7,8-dihydro-6H-pyrazolo[1,5-a]pyrrolo[2,3-e]pyrimidine-6-carboxamide (50 mg, 105.2 μmol) in dichloromethane (2 mL) and methanol (2 mL) were added formaldehyde (34 mg, 420.8 μmol, 37% in water) and sodium triacetoxyborohydride (89 mg, 420.8 μmol). The mixture was stirred at 25° C. for 8 h. The reaction mixture was concentrated under vacuum. The residue was submitted to Prep-HPLC purification and the collected fractions were lyophilized to give (R)-2-chloro-N-(5-chloro-1-(1-methylazetidin-3-yl)-1H-pyrazol-3-yl)-8-methyl-8-(trifluoromethyl)-7,8-dihydro-6H-pyrazolo[1,5-a]pyrrolo[2,3-e]pyrimidine-6-carboxamide (15.0 mg, 29% yield) as a white solid. The enantiomer of Example 163 can be prepared analogously using Method M1 isomer 1.

Example 163: ¹H NMR (400 MHz, DMSO-d₆) δ: 10.01 (s, 1H), 9.30 (s, 1H), 7.04 (s, 1H), 6.56 (s, 1H), 4.94-5.05 (m, 1H), 4.89 (d, J=11.6 Hz, 1H), 4.18 (d, J=11.6 Hz, 1H), 3.74-3.84 (m, 2H), 3.38-3.47 (m, 2H), 2.35 (s, 3H), 1.94 (s, 3H). LC-MS: m/z 489 [M+H]⁺.

### Method M5

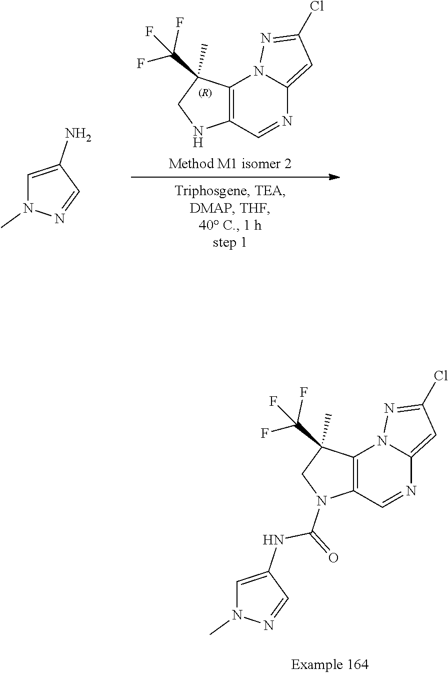

### Examples 164: (R)-2-chloro-8-methyl-N-(1-methyl-1H-pyrazol-4-yl)-8-(trifluoromethyl)-7,8-dihydro-6H-pyrazolo[1,5-a]pyrrolo[2,3-e]pyrimidine-6-carboxamide

### Step 1: (R)-2-chloro-8-methyl-N-(1-methyl-1H-pyrazol-4-yl)-8-(trifluoromethyl)-7,8-dihydro-6H-pyrazolo[1,5-a]pyrrolo[2,3-e]pyrimidine-6-carboxamide

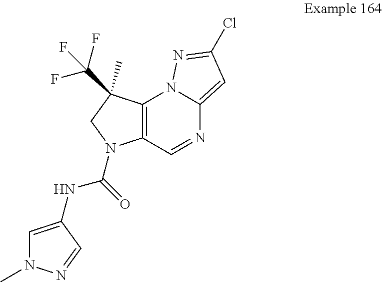 [SMILES: Cn1cc(NC(=O)N2C[C@@](C)(C(F)(F)F)c3c2cnc2cc(Cl)nn32)cn1]

To a stirred solution of 1-methyl-1H-pyrazol-4-amine (21 mg, 217.4 μmol) in tetrahydrofuran (6 mL) were added triphosgene (20 mg, 65.2 μmol) and TEA (17 mg, 168.3 μmol). The resulting mixture was stirred at 28° C. for 0.5 h and then filtered. The resulting filtrate was added to a solution of Method M1 isomer 2 (30 mg, 108.7 μmol) in tetrahydrofuran (1 mL). To this solution was then added N, N-dimethylpyridin-4-amine (27 mg, 217.4 μmol) and TEA (110 mg, 1.1 mmol). The mixture was stirred at 40° C. for 1 h. The mixture was allowed to cool down to 25° C. The reaction mixture was quenched with water (50 mL). The resulting solution was extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate as eluents to afford crude product. The crude product was submitted to Prep-HPLC purification and the collected fractions were lyophilized to give (R)-2-chloro-8-methyl-N-(1-methyl-1H-pyrazol-4-yl)-8-(trifluoromethyl)-7,8-dihydro-6H-pyrazolo[1,5-a]pyrrolo[2,3-e]pyrimidine-6-carboxamide (17.6 mg, 28% yield) as an off-white solid. The enantiomer of Example 164 can be prepared analogously using Method M1 isomer 1.

Example 164: ¹H NMR (400 MHz, DMSO-d₆) δ: 9.35 (s, 1H), 9.13 (br, 1H), 7.81 (d, J=0.8 Hz, 1H), 7.45 (d, J=0.8 Hz, 1H), 7.03 (s, 1H), 4.67 (d, J=11.2 Hz, 1H), 4.16 (d, J=11.6 Hz, 1H), 3.81 (s, 3H), 1.96 (s, 3H). LC-MS: m/z 400 [M+H]⁺.

### Method N5

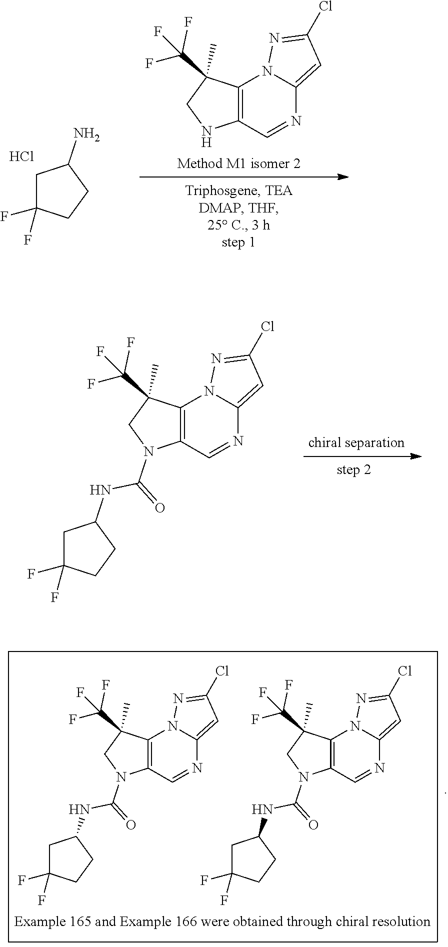

### Examples 165 and 166: Single enantiomers obtained from a racemic mixture containing (R)-2-chloro-N—((R)-3,3-difluorocyclopentyl)-8-methyl-8-(trifluoromethyl)-7,8-dihydro-6H-pyrazolo[1,5-a]pyrrolo[2,3-e]pyrimidine-6-carboxamide and (R)-2-chloro-N—((S)-3,3-difluorocyclopentyl)-8-methyl-8-(trifluoromethyl)-7,8-dihydro-6H-pyrazolo[1,5-a]pyrrolo[2,3-e]pyrimidine-6-carboxamide

### Step 1: (8R)-2-chloro-N-(3,3-difluorocyclopentyl)-8-methyl-8-(trifluoromethyl)-7,8-dihydro-6H-pyrazolo[1,5-a]pyrrolo[2,3-e]pyrimidine-6-carboxamide

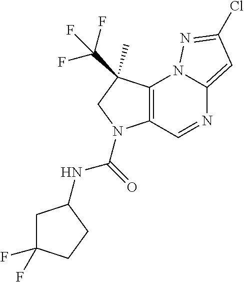 [SMILES: C[C@@]1(C(F)(F)F)CN(C(=O)NC2CCC(F)(F)C2)c2cnc3cc(Cl)nn3c21]

To a stirred solution of Method M1 isomer 2 (40 mg, 144.6 μmol) in tetrahydrofuran (2 mL) were added triphosgene (26 mg, 86.8 μmol) and TEA (22 mg, 216.9 μmol). The resulting mixture was stirred at 25° C. for 1 h and then filtered. The filtrate was added to a solution of 3,3-difluorocyclopentan-1-amine hydrochloride (23 mg, 144.6 μmol) in tetrahydrofuran (1 mL). To this solution were added N,N-dimethylpyridin-4-amine (35 mg, 289.2 μmol) and TEA (146 mg, 1.5 mmol). The mixture was stirred at 25° C. for 3 h. The mixture was concentrated under vacuum. The residue was purified by Prep-HPLC and the collected fractions were lyophilized to afford (8R)-2-chloro-N-(3,3-difluorocyclopentyl)-8-methyl-8-(trifluoromethyl)-7,8-dihydro-6H-pyrazolo[1,5-a]pyrrolo[2,3-e]pyrimidine-6-carboxamide (28 ng, 46% yield) as a white solid. LC-MS: m/z 424 [M+H]⁺.

### Step 2: Separation of enantiomers to obtain (R)-2-chloro-N—((R)-3,3-difluorocyclopentyl)-8-methyl-8-(trifluoromethyl)-7,8-dihydro-6H-pyrazolo[1,5-a]pyrrolo[2,3-e]pyrimidine-6-carboxamide and (R)-2-choro-N—((S)-3,3-difluorocyclopentyl)-8-methyl-8-(trifluoromethyl)-7,8-dihydro-6H-pyrazolo[1,5-a]pyrrolo[2,3-e]pyrimidine-6-carboxamide

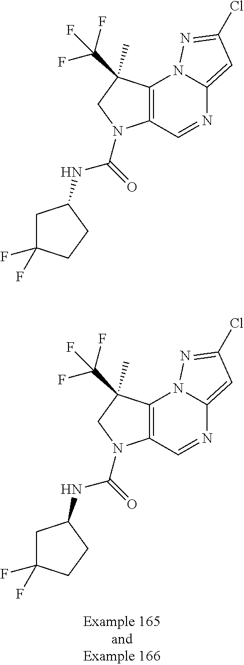 [SMILES: C[C@@]1(C(F)(F)F)CN(C(=O)N[C@@H]2CCC(F)(F)C2)c2cnc3cc(Cl)nn3c21 ; C[C@@]1(C(F)(F)F)CN(C(=O)N[C@H]2CCC(F)(F)C2)c2cnc3cc(Cl)nn3c21]

(8R)-2-chloro-N-(3,3-difluorocyclopentyl)-8-methyl-8-(trifluoromethyl)-7,8-dihydro-6H-pyrazolo[1,5-a]pyrrolo[2,3-e]pyrimidine-6-carboxamide (28 mg, 66 μmol) was submitted to chiral HPLC purification: Column: CHIRAL ART Cellulose-SB, 2*25 cm, 5 um; Mobile Phase A:Hex(0.5% 2M NH₃-MeOH)—HPLC, Mobile Phase B:EtOH—HPLC; Flow rate: 20 mL/min; Gradient: 8 B to 8 B in 15 min; 220/254 nm; RT1:10.681; RT2:12.396; Injection Volume: 0.5 ml; Number Of Runs: 3; The first eluting isomer was concentrated and lyophilized to afford Example 165 as a white solid (6 mg, 10% yield). The second eluting isomer was concentrated and lyophilized to afford Example 166 as a white solid (3 mg, 5% yield). The corresponding stereoisomers of Examples 165 and 166 can be prepared analogously using Method M1 isomer 1. Examples 165 and 166 are diastereomers, wherein the stereocenter attached to the trifluoromethyl is absolute and the cyclopentyl stereocenter is relative (i.e., the cyclopentyl stereocenter in one of Examples 165 and 166 is (S), and the cyclopentyl stereocenter in the other of Examples 165 and 166 is (R)).

Example 165: ¹H NMR (400 MHz, DMSO-d₆) δ: 9.29 (s, 1H), 7.20 (d, J=6.8 Hz, 1H), 7.01 (s, 1H), 4.56 (d, J=11.6 Hz, 1H), 4.18-4.27 (m, 1H), 4.00 (d, J=11.6 Hz, 1H), 2.43-2.52 (m, 1H), 1.99-2.32 (m, 4H), 1.92 (s, 3H), 1.78-1.85 (m, 1H). LC-MS: m/z 424 [M+H]⁺.

Example 166: ¹H NMR (400 MHz, DMSO-d₆) δ: 9.29 (s, 1H), 7.20 (d, J=6.8 Hz, 1H), 7.01 (s, 1H), 4.56 (d, J=11.6 Hz, 1H), 4.20-4.25 (m, 1H), 4.00 (d, J=11.6 Hz, 1H), 2.44-2.51 (m, 1H), 1.98-2.32 (m, 4H), 1.93 (s, 3H), 1.78-1.86 (m, 1H). LC-MS: m/z 424 [M+H]⁺.

### Method O5

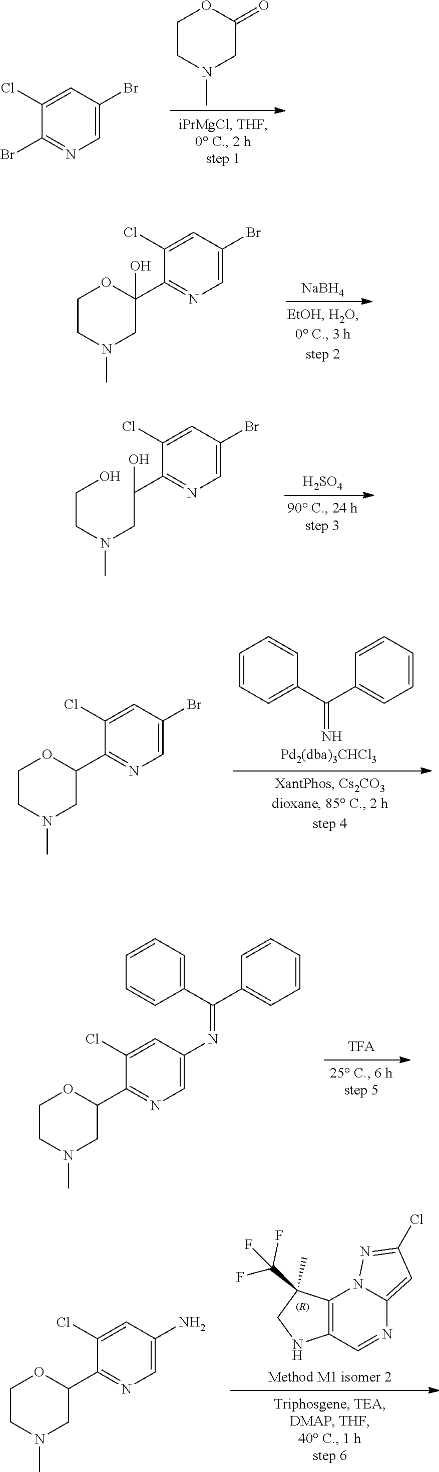

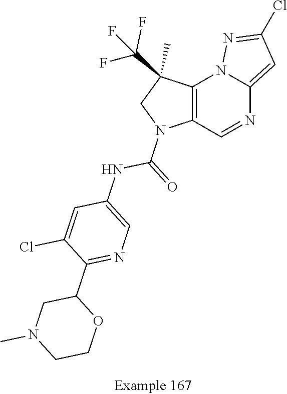 [SMILES: CN1CCOC(c2ncc(NC(=O)N3C[C@@](C)(C(F)(F)F)c4c3cnc3cc(Cl)nn43)cc2Cl)C1]

### Example 167: (8R)-2-chloro-N-(5-chloro-6-(4-methylmorpholin-2-yl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 2-(5-bromo-3-chloropyridin-2-yl)-4-methylmorpholin-2-ol

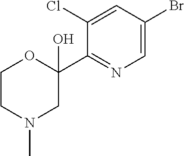 [SMILES: CN1CCOC(O)(c2ncc(Br)cc2Cl)C1]

To a stirred solution of 2,5-dibromo-3-chloropyridine (18.8 g, 69.3 mmol) in tetrahydrofuran (400 mL) was added isopropylmagnesium chloride (35 mL, 69.3 mmol, 2 M in tetrahydrofuran) at 0° C. under nitrogen atmosphere. The reaction mixture was stirred at 0° C. for 0.5 h, and then 4-methylmorpholin-2-one (8 g, 69.3 mmol) was added dropwise at 0° C. The reaction mixture was stirred at 0° C. for 2 h. The reaction mixture was quenched with saturated aqueous NH₄Cl solution (200 mL) and extracted with ethyl acetate (3×200 mL). The combined organic layers were washed with brine (100 mL), dried over anhydrous sodium sulfate and concentrated under vacuum. The residue was purified by column chromatography on silica gel using 90% dichloromethane and 10% methanol as eluents to afford 2-(5-bromo-3-chloropyridin-2-yl)-4-methylmorpholin-2-ol (12 g, 50% yield) as a yellow oil. ¹H NMR (300 MHz, DMSO-d₆) δ: 8.45 (d, J=2.1 Hz, 1H), 8.05 (d, J=2.1 Hz, 1H), 6.84 (br, 1H), 3.97-4.12 (m, 2H), 2.60-2.79 (m, 2H), 2.12-2.28 (m, 2H), 2.19 (s, 3H). LC-MS: m/z 307 [M+H]⁺.

### Step 2: 1-(5-bromo-3-chloropyridin-2-yl)-2-((2-hydroxyethyl)(methyl) amino)ethan-1-ol

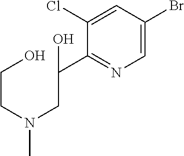 [SMILES: CN(CCO)CC(O)c1ncc(Br)cc1Cl]

To a stirred solution of 2-(5-bromo-3-chloropyridin-2-yl)-4-methylmorpholin-2-ol (6 g, 19.5 mmol) in ethanol (100 mL) and water (40 mL) was added NaBH₄ (2.9 g, 78 mmol) under 0° C. The reaction mixture was stirred at 0° C. for 3 h. The resulting mixture was concentrated under vacuum. The residue was diluted with water (100 mL). The resulting solution was extracted with ethyl acetate (3×100 mL). The combined organic layers were dried over anhydrous sodium sulfate and concentrated under vacuum to afford 1-(5-bromo-3-chloropyridin-2-yl)-2-((2-hydroxyethyl)(methyl) amino)ethan-1-ol (5 g, 83% yield) as a yellow oil. LC-MS: m/z 309 [M+H]⁺.

### Step 3: 2-(5-bromo-3-chloropyridin-2-yl)-4-methylmorpholine

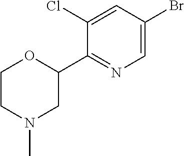

A solution of 1-(5-bromo-3-chloropyridin-2-yl)-2-((2-hydroxyethyl)(methyl) amino)ethan-1-ol (4 g, 12.9 mmol) in concentrated sulfuric acid (40 mL) was stirred at 90° C. for 24 h. The reaction mixture was cooled to 25° C. The reaction solution was poured into ice water (200 mL), and the pH was adjusted to 6-7 with aqueous NaOH solution (4 M) and extracted with ethyl acetate (3×200 mL). The combined organic layers were washed with brine (100 mL), dried over anhydrous sodium sulfate and concentrated under vacuum. The residue was purified by column chromatography on silica gel using 90% dichloromethane and 10% methanol as eluents to afford 2-(5-bromo-3-chloropyridin-2-yl)-4-methylmorpholine (2 g, 52% yield) as a yellow oil. ¹H NMR (400 MHz, Chloroform-d) δ: 8.26 (d, J=2.0 Hz, 1H), 7.79 (d, J=2.0 Hz, 1H), 4.60-4.62 (m, 1H), 4.03-4.07 (m, 1H), 3.82-3.88 (m, 1H), 2.92 (d, J=11.6 Hz, 1H), 2.78 (d, J=11.6 Hz, 1H), 2.37 (s, 3H), 2.21-2.32 (m, 1H), 1.99-2.05 (m, 1H). LC-MS: m/z 291 [M+H]⁺.

### Step 4: N-(5-chloro-6-(4-methylmorpholin-2-yl)pyridin-3-yl)-1,1-diphenylmethanimine

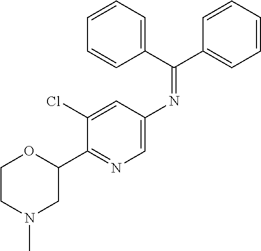 [SMILES: CN1CCOC(c2ncc(N=C(c3ccccc3)c3ccccc3)cc2Cl)C1]

To a stirred solution of 2-(5-bromo-3-chloropyridin-2-yl)-4-methylmorpholine (1 g, 3.4 mmol) in dioxane (20 mL) was added diphenylmethanimine (621 mg, 3.4 mmol), Xantphos (396 mg, 685 μmol), Pd₂(dba)₃CHCl₃ (355 mg, 343 μmol) and Cs₂CO₃ (2.2 g, 6.8 mmol). The reaction mixture was stirred at 85° C. for 2 h under nitrogen. The mixture was cooled to 25° C. The resulting mixture was filtered. The filter cake was washed with ethyl acetate (3×20 mL). The filtrate was concentrated under reduced pressure. The residue was purified by column chromatography on silica gel using 90% dichloromethane and 10% methanol as eluents to afford N-(5-chloro-6-(4-methylmorpholin-2-yl)pyridin-3-yl)-1,1-diphenylmethanimine (1 g, 66% yield) as a yellow oil. LC-MS: m/z 392 [M+H]⁺.

### Step 5: 5-chloro-6-(4-methylmorpholin-2-yl)pyridin-3-amine

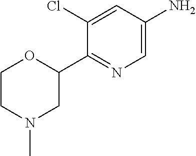

A solution of N-(5-chloro-6-(4-methylmorpholin-2-yl)pyridin-3-yl)-1,1-diphenylmethanimine (400 mg, 1.0 mmol) in TFA (20 mL) was stirred at 25° C. for 6 h. The mixture was concentrated under vacuum. The residue was diluted with water (20 mL), and the pH was adjusted to 6-7 with saturated aqueous NaHCO₃ solution. The resulting solution was extracted with ethyl acetate (3×100 mL). The combined organic layers were washed with brine (100 mL), dried over anhydrous sodium sulfate and concentrated under vacuum. The residue was purified by column chromatography on silica gel using 90% dichloromethane and 10% methanol as eluents to afford 5-chloro-6-(4-methylmorpholin-2-yl)pyridin-3-amine (100 mg, 30% yield) as a brown oil. LC-MS: m/z 228 [M+H]⁺.

### Step 6: (8R)-2-chloro-N-(5-chloro-6-(4-methylmorpholin-2-yl)pyridin-3-yl)-8-methyl-8-(trifluoromethyl)-7,8-dihydro-6H-pyrazolo[1,5-a]pyrrolo[2,3-e]pyrimidine-6-carboxamide

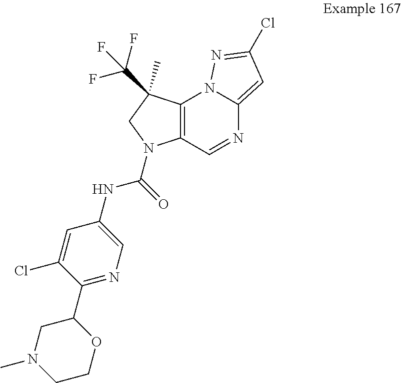 [SMILES: CN1CCOC(c2ncc(NC(=O)N3C[C@@](C)(C(F)(F)F)c4c3cnc3cc(Cl)nn43)cc2Cl)C1]

To a stirred solution of 5-chloro-6-(4-methylmorpholin-2-yl)pyridin-3-amine (82 mg, 360 μmol) in tetrahydrofuran (8 mL) were added triphosgene (32 mg, 108 μmol) and TEA (36 mg, 360 μmol) at 25° C. The resulting mixture was stirred at 25° C. for 0.5 h and then filtered. The filtrate was added to a solution of Method M1 isomer 2 (50 mg, 180 μmol) in tetrahydrofuran (2 mL). To this solution were then added N, N-dimethylpyridin-4-amine (44 mg, 360 μmol) and TEA (182 mg, 1.8 mmol). The mixture was stirred at 40° C. for 1 h. The reaction mixture was quenched with water (50 mL), and the resulting solution was extracted with ethyl acetate (3×50 mL). The combined organic layers were washed with brine (20 mL), dried over anhydrous sodium sulfate and concentrated under vacuum. The residue was submitted to Prep-HPLC purification and the collected fractions were lyophilized to give (8R)-2-chloro-N-(5-chloro-6-(4-methylmorpholin-2-yl)pyridin-3-yl)-8-methyl-8-(trifluoromethyl)-7,8-dihydro-6H-pyrazolo[1,5-a]pyrrolo[2,3-e]pyrimidine-6-carboxamide (12 mg, 12% yield) as a white solid. The enantiomer of Example 167 can be prepared analogously using Method M1 isomer 1.

Example 167: ¹H NMR (400 MHz, Methanol-d₄) δ: 9.33 (s, 1H), 8.34 (s, 1H), 8.00 (d, J=2.0 Hz, 1H), 6.80 (s, 1H), 4.77 (d, J=11.7 Hz, 1H), 4.62-4.65 (m, 1H), 4.22 (d, J=11.7 Hz, 1H), 4.05-4.09 (m, 1H), 3.79-3.87 (m, 1H), 3.02 (d, J=11.7 Hz, 1H), 2.81 (d, J=11.7 Hz, 1H), 2.37 (s, 3H), 2.25-2.33 (m, 1H), 2.03-2.06 (m, 1H), 2.05 (s, 3H). LC-MS: m/z 530 [M+H]⁺.

### Method P5

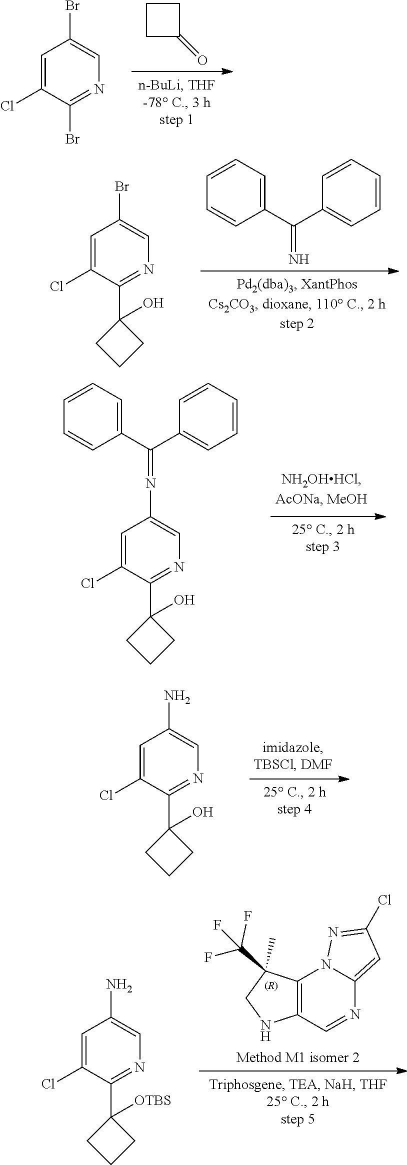

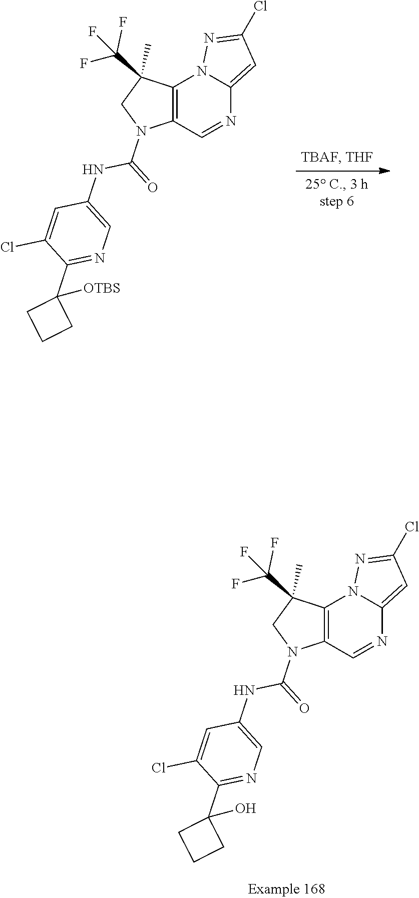 [SMILES: CC(C)(C)[Si](C)(C)OC1(c2ncc(NC(=O)N3C[C@@](C)(C(F)(F)F)c4c3cnc3cc(Cl)nn43)cc2Cl)CCC1 ; C[C@@]1(C(F)(F)F)CN(C(=O)Nc2cnc(C3(O)CCC3)c(Cl)c2)c2cnc3cc(Cl)nn3c21]

### Example 168: (R)-2-chloro-N-(5-chloro-(1-hydroxycyclobutyl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 1-(5-bromo-3-chloropyridin-2-yl)cyclobutan-1-ol

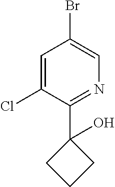 [SMILES: OC1(c2ncc(Br)cc2Cl)CCC1]

To a stirred solution of 2,5-dibromo-3-chloropyridine (10.00 g, 36.8 mmol) in tetrahydrofuran (60 mL) was added n-BuLi (17.7 mL, 44.2 mmol, 2.5 M in hexanes) dropwise at −78° C. The reaction mixture was stirred at −78° C. for 1 h. and then cyclobutanone (2.58 g, 36.8 mmol) was added into the mixture at −78° C. The reaction mixture was stirred at −78° C. for 3 h. The reaction mixture was quenched by brine (200 mL) and extracted with ethyl acetate (3×200 mL). The combined organic layers were dried over anhydrous sodium sulfate and concentrated under vacuum. The residue was purified by column chromatography on silica gel using 80% petroleum ether and 20% ethyl acetate as eluents to afford 1-(5-bromo-3-chloropyridin-2-yl)cyclobutan-1-ol (1 g, 10% yield) as a yellow oil. LC-MS: m/z 262 [M+H]⁺.

### Step 2: 1-(3-chloro-5-((diphenylmethylene)amino)pyridin-2-yl)cyclobutan-1-ol

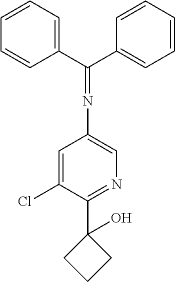 [SMILES: OC1(c2ncc(N=C(c3ccccc3)c3ccccc3)cc2Cl)CCC1]

To a stirred solution of 1-(5-bromo-3-chloropyridin-2-yl)cyclobutan-1-ol (500 mg, 1.9 mmol) and diphenylmethanimine (345 mg, 1.9 mmol) in dioxane (3 mL) were added Pd₂(dba)₃ (394 mg, 380.9 μmol), XantPhos (330 mg, 571.3 μmol) and Cs₂CO₃ (1.86 g, 5.7 mmol). The resulting mixture was stirred at 110° C. for 2 h under nitrogen atmosphere. The reaction mixture was concentrated under vacuum. The residue was diluted with water (50 mL), and the resulting solution was extracted with ethyl acetate (3×50 mL). The combined organic layers were dried over anhydrous sodium sulfate and concentrated under vacuum. The residue was purified by column chromatography on silica gel using 75% petroleum ether and 25% ethyl acetate to afford 1-(3-chloro-5-((diphenylmethylene)amino)pyridin-2-yl)cyclobutan-1-ol (375 mg, 86% yield) as a yellow solid. LC-MS: m/z 363 [M+H]⁺.

### Step 3: 1-(5-amino-3-chloropyridin-2-yl)cyclobutan-1-ol

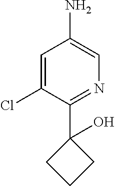 [SMILES: Nc1cnc(C2(O)CCC2)c(Cl)c1]

To a stirred solution of 1-(3-chloro-5-((diphenylmethylene)amino)pyridin-2-yl)cyclobutan-1-ol (400 mg, 1.1 mmol) in methanol (10 mL) were added hydroxylamine hydrochloride (153 mg, 2.2 mmol) and sodium acetate (375 mg, 2.7 mmol). The reaction mixture was stirred at 25° C. for 2 h. The reaction mixture was concentrated under vacuum. The residue was purified by column chromatography on silica gel using 90% dichloromethane and 10% methanol as eluents to afford 1-(5-amino-3-chloropyridin-2-yl)cyclobutan-1-ol (200 mg, 91% yield) as a yellow solid. LC-MS: m/z 199 [M+H]⁺.

### Step 4: 6-(1-((tert-butyldimethylsilyl)oxy)cyclobutyl)-5-chloropyridin-3-amine

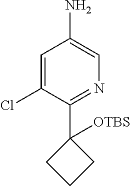 [SMILES: CC(C)(C)[Si](C)(C)OC1(c2ncc(N)cc2Cl)CCC1]

To a stirred solution of 1-(5-amino-3-chloropyridin-2-yl)cyclobutan-1-ol (100 mg, 503.4 μmol) in N,N-Dimethylformamide (10 mL) were added imidazole (51 mg, 755.1 μmol) and TBSCl (91 mg, 604.1 μmol) at 0° C. under nitrogen atmosphere. The resulting mixture was stirred at 25° C. for 2 h. The reaction mixture was concentrated under vacuum. The residue was quenched with water (80 mL). The resulting solution was extracted with ethyl acetate (2×80 mL). The combined organic layers were dried over anhydrous sodium sulfate and concentrated under vacuum. The residue was purified by column chromatography on silica gel using 60% petroleum ether and 40% ethyl acetate as eluents to afford 6-(1-((tert-butyldimethylsilyl)oxy)cyclobutyl)-5-chloropyridin-3-amine (150 mg, 95% yield) as a yellow oil. LC-MS: m/z 313 [M+H]⁺.

### Step 5: (R)—N-(6-(1-((tert-butyldimethylsilyl)oxy)cyclobutyl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide

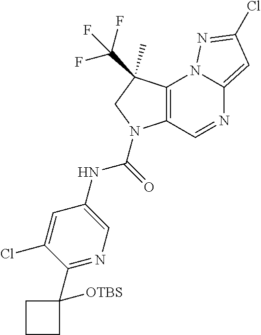 [SMILES: CC(C)(C)[Si](C)(C)OC1(c2ncc(NC(=O)N3C[C@@](C)(C(F)(F)F)c4c3cnc3cc(Cl)nn43)cc2Cl)CCC1]

To a stirred mixture of Method M1 isomer 2 (22 mg, 79.9 μmol) in tetrahydrofuran (5 mL) was added TEA (24 mg, 239.7 μmol) and triphosgene (24 mg, 79.9 μmol). The resulting mixture was stirred at 25° C. for 1 h and then filtered. The filtrate was added to a solution of 6-(1-((tert-butyldimethylsilyl)oxy)cyclobutyl)-5-chloropyridin-3-amine (50 mg, 159.1 μmol) in tetrahydrofuran (5 mL). To this solution was added NaH (12 mg, 319.5 μmol, 60% in mineral oil). The mixture was stirred at 25° C. for 2 h. The reaction mixture was quenched with water (10 mL). The resulting solution was extracted with ethyl acetate (3×10 mL). The combined organic layers were dried over anhydrous sodium sulfate and concentrated under vacuum. The residue was purified by Prep-TLC using 80% petroleum ether and 20% ethyl acetate as eluents to afford (R)—N-(6-(1-((tert-butyldimethylsilyl)oxy)cyclobutyl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide (13 mg, 13% yield) as a white solid. LC-MS: m/z 615 [M+H]⁺.

### Step 6: (R)-2-chloro-N-(5-chloro-6-(1-hydroxycyclobutyl)pyridin-3-yl)-8-methyl-8-(trifluoromethyl)-7,8-dihydro-6H-pyrazolo[1,5-a]pyrrolo[2,3-e]pyrimidine-6-carboxamide

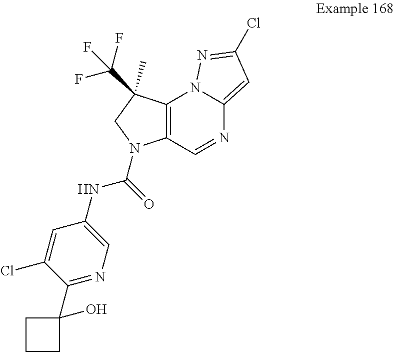 [SMILES: C[C@@]1(C(F)(F)F)CN(C(=O)Nc2cnc(C3(O)CCC3)c(Cl)c2)c2cnc3cc(Cl)nn3c21]

To a stirred mixture of (R)—N-(6-(1-((tert-butyldimethylsilyl)oxy)cyclobutyl)-5-chloropyridin-3-yl)-2-chloro-8-methyl-8-(trifluoromethyl)-7,8-dihydro-6H-pyrazolo[1,5-a]pyrrolo[2,3-e]pyrimidine-6-carboxamide (13 mg, 21.1 μmol) in tetrahydrofuran (1 mL) was added TBAF (1 mL, 1 M in THF). The reaction mixture was stirred at 25° C. for 3 h. The mixture was concentrated under vacuum. The residue was purified by Prep-TLC using 95% dichloromethane and 5% methanol as eluents to afford a crude product which was purified by Prep-HPLC and the collected fractions were lyophilized to afford (R)-2-chloro-N-(5-chloro-6-(1-hydroxycyclobutyl)pyridin-3-yl)-8-methyl-8-(trifluoromethyl)-7,8-dihydro-6H-pyrazolo[1,5-a]pyrrolo[2,3-e]pyrimidine-6-carboxamide (2.6 mg, 24% yield) as a light-yellow solid. The enantiomer of Example 168 can be prepared analogously using Method M1 isomer 1.

Example 168: ¹H NMR (300 MHz, DMSO-d₆) δ: 9.27 (s, 1H), 8.47 (s, 1H), 8.01 (d, J=1.8 Hz 1H) 7.04 (s, 1H), 5.87-5.91 (m, 1H), 4.79 (d, J=12.0 Hz, 1H), 4.21 (d, J=12.0 Hz, 1H), 2.28-2.35 (m, 4H), 1.91-1.97 (m, 4H), 1.69-1.75 (m, 1H). LC-MS: m/z 501 [M+H]⁺.

### Method Q5

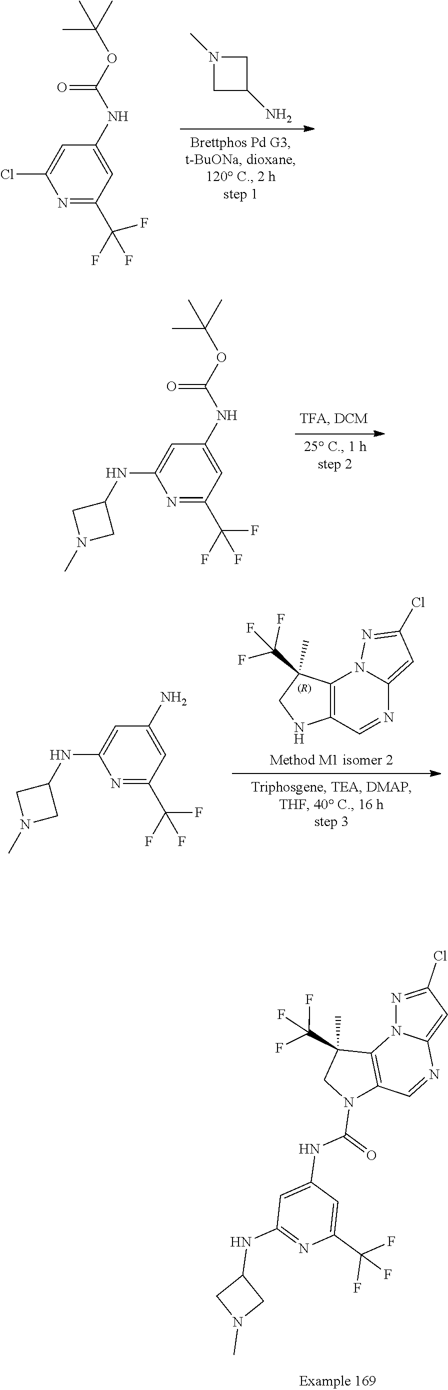

### Example 169: (R)-2-chloro-8-methyl-N-(2-((1-methylazetidin-3-yl)amino)-6-(trifluoromethyl)pyridin-4-yl)-8-(trifluoromethyl)-7,8-dihydro-6H-pyrazolo[1,5-a]pyrrolo[2,3-e]pyrimidine-6-carboxamide

### Step 1: tert-butyl (2-((1-methylazetidin-3-yl)amino)-6-(trifluoromethyl)pyridin-4-yl)carbamate

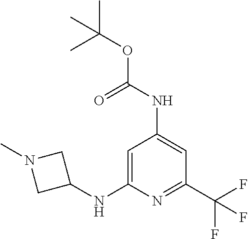 [SMILES: CN1CC(Nc2cc(NC(=O)OC(C)(C)C)cc(C(F)(F)F)n2)C1]

To a stirred solution of tert-butyl (2-chloro-6-(trifluoromethyl)pyridin-4-yl)carbamate (300 mg, 1.0 mmol) in dioxane (10 mL) was added sodium tert-butoxide (480 mg, 5.0 mmol), 1-methylazetidin-3-amine (610 mg, 7.1 mmol) and Brettphos Pd G3 (91 mg, 101.4 μmol) at 25° C. under nitrogen atmosphere. The resulting mixture was stirred at 120° C. for 2 h. The reaction mixture was cooled to 25° C. The solid was filtered out. The filtrate was concentrated under vacuum. The residue was purified by column chromatography on silica gel using 90% dichloromethane and 10% methanol as eluents to afford tert-butyl (2-((1-methylazetidin-3-yl)amino)-6-(trifluoromethyl)pyridin-4-yl)carbamate (130 mg, 37% yield) as a white solid. LC-MS: m/z 347 [M+H]⁺.

### Step 2: N²-(1-methylazetidin-3-yl)-6-(trifluoromethyl)pyridine-2,4-diamine

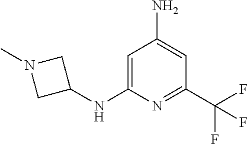 [SMILES: CN1CC(Nc2cc(N)cc(C(F)(F)F)n2)C1]

To a stirred solution of tert-butyl (2-((1-methylazetidin-3-yl)amino)-6-(trifluoromethyl)pyridin-4-yl)carbamate (130 mg, 375.7 μmol) in dichloromethane (12 mL) was added TFA (3 mL). The mixture was stirred at 25° C. for 1 h. The resulting mixture was concentrated under vacuum. The residue was diluted with saturated aqueous NaHCO₃ solution (30 mL). The resulting solution was extracted with ethyl acetate (3×50 mL). The combined organic layers were dried over anhydrous sodium sulfate and concentrated under vacuum. The residue was purified by column chromatography on silica gel using 80% dichloromethane and 20% methanol as eluents to afford N²-(1-methylazetidin-3-yl)-6-(trifluoromethyl)pyridine-2,4-diamine (80 mg, 87% yield) as a white solid. LC-MS: m/z 247 [M+H]⁺.

### Step 3: (R)-2-chloro-8-methyl-N-(2-((1-methylazetidin-3-yl)amino)-6-(trifluoromethyl)pyridin-4-yl)-8-(trifluoromethyl)-7,8-dihydro-6H-pyrazolo[1,5-a]pyrrolo[2,3-e]pyrimidine-6-carboxamide

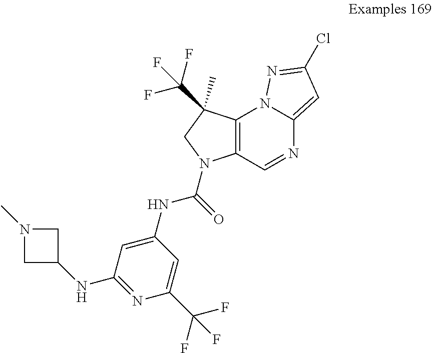 [SMILES: CN1CC(Nc2cc(NC(=O)N3C[C@@](C)(C(F)(F)F)c4c3cnc3cc(Cl)nn43)cc(C(F)(F)F)n2)C1]

To a stirred solution of N²-(1-methylazetidin-3-yl)-6-(trifluoromethyl)pyridine-2,4-diamine (30 mg, 121.9 μmol) in tetrahydrofuran (8 mL) were added triphosgene (21 mg, 73.2 μmol) and TEA (31 mg, 304.7 μmol). The resulting mixture was stirred at 40° C. for 0.5 h and then filtered. The resulting filtrate was added to a solution of Method M1 isomer 2 (34 mg, 121.9 μmol) in tetrahydrofuran (2 mL). To this solution were then added TEA (121 mg, 1.2 mmol) and N, N-dimethylpyridin-4-amine (18 mg, 146.3 μmol). The mixture was stirred at 40° C. for 16 h. The solvent was concentrated under vacuum. The residue was purified by Prep-TLC using 90% dichloromethane and 10% methanol as eluents to afford 30 mg of crude product which was purified by Prep-HPLC purification and the collected fractions were lyophilized to give (R)-2-chloro-8-methyl-N-(2-((1-methylazetidin-3-yl)amino)-6-(trifluoromethyl)pyridin-4-yl)-8-(trifluoromethyl)-7,8-dihydro-6H-pyrazolo[1,5-a]pyrrolo[2,3-e]pyrimidine-6-carboxamide (12 mg 18% yield) as a white solid. The enantiomer of Example 169 can be prepared analogously using Method M1 isomer 1.

Example 169: ¹H NMR (400 MHz, Methanol-d₄) δ: 8.62 (d, J=4.4 Hz, 1H), 8.55 (br s, 1H), 6.74-6.77 (m, 2H), 5.99-6.02 (m, 1H), 4.58-4.61 (m, 2H), 4.34-4.41 (m, 2H), 4.15-4.25 (m, 2H), 3.75-3.88 (m, 1H), 3.42-3.55 (m, 1H), 3.17 (s, 3H), 1.96 (s, 3H). LC-MS: m/z 549 [M+H]⁺.

### Method R5

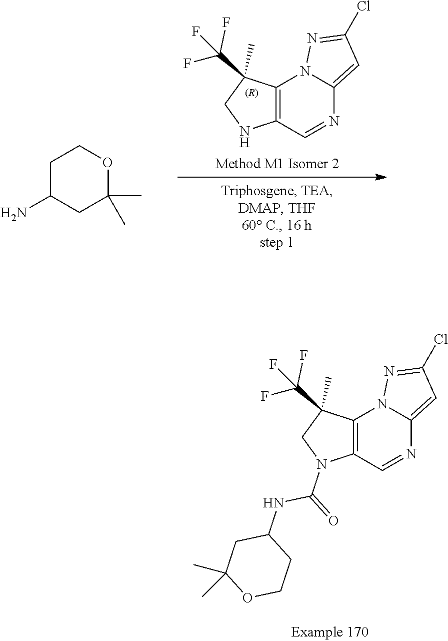

### Example 170: (8R)-2-chloro-N-(2,2-dimethyltetrahydro-2H-pyran-4-yl)-8-methyl-8 (trifluoromethyl)-7,8-dihydro-6H-pyrazolo[1,5-a]pyrrolo[2,3-e]pyrimidine-6-carboxamide

### Step 1: (8R)-2-chloro-N-(2,2-dimethyltetrahydro-2H-pyran-4-yl)-8-methyl-8-(trifluoromethyl)-7,8-dihydro-6H-pyrazolo[1,5-a]pyrrolo[2,3-e]pyrimidine-6-carboxamide

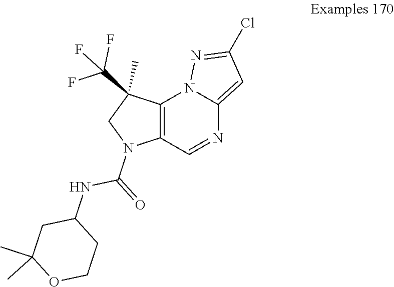 [SMILES: CC1(C)CC(NC(=O)N2C[C@@](C)(C(F)(F)F)c3c2cnc2cc(Cl)nn32)CCO1]

To a stirred solution of 2,2-dimethyltetrahydro-2H-pyran-4-amine (56 mg, 434.1 μmol) in tetrahydrofuran (8 mL) were added triphosgene (77 mg, 260.1 μmol) and TEA (65 mg, 650.1 μmol). The resulting mixture was stirred at 25° C. for 0.5 h and then filtered. The filtrate was added to a solution of Method M1 isomer 2 (39 mg, 140.8 μmol) in tetrahydrofuran (2 mL). To this solution were then added TEA (438 mg, 4.3 mmol) and N,N-dimethylpyridin-4-amine (105 mg, 853.6 μmol). The mixture was stirred at 60° C. for 16 h. The mixture was quenched with water (10 mL), and the resulting solution was extracted with ethyl acetate (3×10 mL). The combined organic layers were washed with brine (20 mL), dried over anhydrous sodium sulfate and concentrated under vacuum. The residue was submitted to Prep-HPLC purification and the collected fractions were lyophilized to give (8R)-2-chloro-N-(2,2-dimethyltetrahydro-2H-pyran-4-yl)-8-methyl-8-(trifluoromethyl)-7,8-dihydro-6H-pyrazolo[1,5-a]pyrrolo[2,3-e]pyrimidine-6-carboxamide (25 mg, 13% yield) as a white solid. The enantiomer of Example 170 can be prepared analogously using Method M1 isomer 1.

Example 170: ¹H NMR (400 MHz, DMSO-d₆) δ: 9.29 (s, 1H), 6.98 (s, 1H), 6.85 (br, 1H), 4.54-4.57 (m, 1H), 3.85-4.01 (m, 2H), 3.64-3.70 (m, 2H), 1.92 (s, 3H), 1.65-1.80 (m, 2H), 1.26-1.47 (m, 2H), 1.18 (s, 3H), 1.15 (s, 3H). LC-MS: m/z 432 [M+H]⁺.

### Method S5

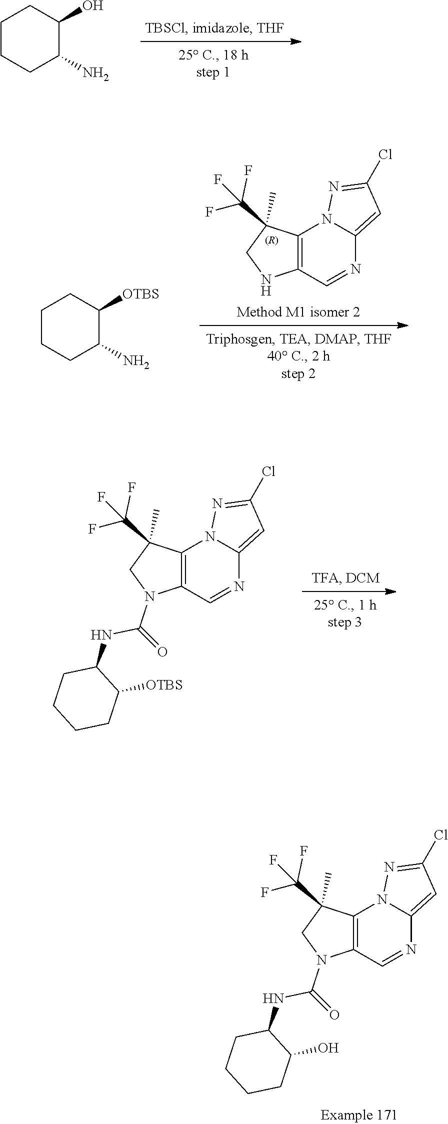

### Example 171: (R)-2-chloro-N-((1R,2R)-2-hydroxycyclohexyl)-8-methyl-8-(trifluoromethyl)-7,8-dihydro-6H-pyrazolo[1,5-a]pyrrolo[2,3-e]pyrimidine-6-carboxamide

### Step 1: (1R,2R)-2-((tert-butyldimethylsilyl)oxy)cyclohexan-1-amine

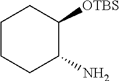 [SMILES: CC(C)(C)[Si](C)(C)O[C@@H]1CCCC[C@H]1N]

To a stirred solution of (1R,2R)-2-aminocyclohexan-1-ol (500 mg, 4.3 mmol) in tetrahydrofuran (10 mL) were added imidazole (880 mg, 12.9 mmol) and tert-butylchlorodimethylsilane (780 mg, 5.2 mmol) at 0° C. The reaction mixture was stirred at 25° C. for 18 h. The mixture was concentrated under vacuum. The residue was purified by Prep-HPLC and the collected fractions were concentrated to afford (1R,2R)-2-((tert-butyldimethylsilyl)oxy)cyclohexan-1-amine (190 mg, 11% yield) as a yellow oil. LC-MS: m/z 230 [M+H]⁺.

### Step 2: (R)—N-((1R,2R)-2-((tert-butyldimethylsilyl)oxy)cyclohexyl)-2-chloro-8-methyl-8-(trifluoromethyl)-7,8-dihydro-6H-pyrazolo[1,5-a]pyrrolo[2,3-e]pyrimidine-6-carboxamide

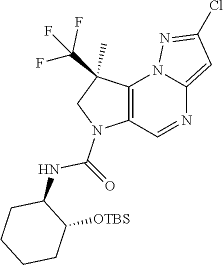 [SMILES: CC(C)(C)[Si](C)(C)O[C@@H]1CCCC[C@H]1NC(=O)N1C[C@@](C)(C(F)(F)F)c2c1cnc1cc(Cl)nn21]

To a stirred solution of (1R,2R)-2-((tert-butyldimethylsilyl)oxy)cyclohexan-1-amine (62 mg, 162.6 μmol) in tetrahydrofuran (3 mL) were added triphosgene (29 mg, 97.7 μmol) and TEA (33 mg, 326.1 μmol). The resulting mixture was stirred at 40° C. for 1 h and then filtered. The filtrate was added to a solution of Method M1 isomer 2 (30 mg, 108.4 μmol) in tetrahydrofuran (3 mL). To this solution were added N,N-dimethylpyridin-4-amine (26 mg, 212.8 μmol) and TEA (110 mg, 1.09 mmol). The mixture was stirred at 40° C. for 2 h. The resulting mixture was filtered. The filtrate was concentrated under vacuum. The residue was purified by column chromatography on silica gel using 75% petroleum ether and 25% ethyl acetate to afford (R)—N-((1R,2R)-2-((tert-butyldimethylsilyl)oxy)cyclohexyl)-2-chloro-8-methyl-8-(trifluoromethyl)-7,8-dihydro-6H-pyrazolo[1,5-a]pyrrolo[2,3-e]pyrimidine-6-carboxamide (45 mg, 78% yield) as a light-yellow solid. LC-MS: m/z 532 [M+H]⁺.

### Step 3: (R)-2-chloro-N-((1R,2R)-2-hydroxycyclohexyl)-8-methyl-8-(trifluoromethyl)-7,8-dihydro-6H-pyrazolo[1,5-a]pyrrolo[2,3-e]pyrimidine-6-carboxamide

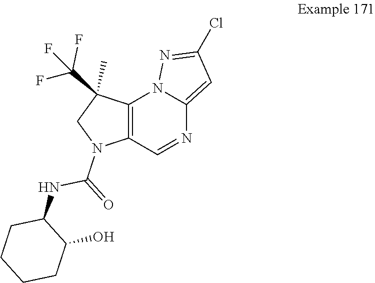 [SMILES: C[C@@]1(C(F)(F)F)CN(C(=O)N[C@@H]2CCCC[C@H]2O)c2cnc3cc(Cl)nn3c21]

To a mixture of (R)—N-((1R,2R)-2-((tert-butyldimethylsilyl)oxy)cyclohexyl)-2-chloro-8-methyl-8-(trifluoromethyl)-7,8-dihydro-6H-pyrazolo[1,5-a]pyrrolo[2,3-e]pyrimidine-6-carboxamide (30 mg, 56 μmol) in dichloromethane (2 mL) was added TFA (1 mL). The mixture was stirred at 25° C. for 1 h. The mixture was concentrated under vacuum. The residue was diluted with saturated aqueous NaHCO₃ solution (5 mL), and the resulting mixture was extracted with dichloromethane (3×5 mL). The combined organic layers were dried over anhydrous sodium sulfate and concentrated under vacuum. The residue was purified by Prep-HPLC and the collected fractions were lyophilized to afford (R)-2-chloro-N-((1R,2R)-2-hydroxycyclohexyl)-8-methyl-8-(trifluoromethyl)-7,8-dihydro-6H-pyrazolo[1,5-a]pyrrolo[2,3-e]pyrimidine-6-carboxamide (18.3 mg, 77% yield) as an off-white solid. The enantiomer of Example 171 can be prepared analogously using Method M1 isomer 1.

Example 171: ¹H NMR (400 MHz, DMSO-d₆) δ: 9.30 (s, 1H), 6.98 (s, 1H), 6.76 (d, J=7.6 Hz, 1H), 4.71 (d, J=4.4 Hz, 1H), 4.58 (d, J=11.6 Hz, 1H), 3.99 (d, J=11.6 Hz, 1H), 3.32-3.38 (m, 2H), 1.92 (s, 3H), 1.79-1.90 (m, 2H), 1.60-1.67 (m, 2H), 1.18-1.24 (m, 4H). LC-MS: m/z 418 [M+H]⁺.

### Method T5

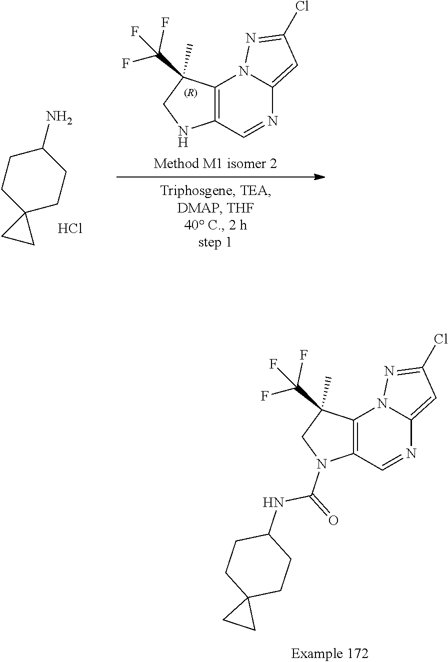

### Example 172: (R)-2-chloro-8-methyl-N-(spiro[2.5]octan-6-yl)-8-(trifluoromethyl)-7,8-dihydro-6H-pyrazolo[1,5-a]pyrrolo[2,3-e]pyrimidine-6-carboxamide

### Step 1: (R)-2-chloro-8-methyl-N-(spiro[2.5]octan-6-yl)-8-(trifluoromethyl)-7,8-dihydro-6H-pyrazolo[1,5-a]pyrrolo[2,3-e]pyrimidine-6-carboxamide

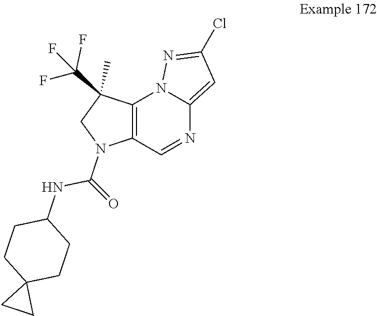 [SMILES: C[C@@]1(C(F)(F)F)CN(C(=O)NC2CCC3(CC2)CC3)c2cnc3cc(Cl)nn3c21]

To a stirred solution of Method M1 isomer 2 (34 mg, 123.7 μmol) in tetrahydrofuran (2 mL) were added triphosgene (21 mg, 73.8 μmol) and TEA (25 mg, 247.4 μmol). The resulting mixture was stirred at 40° C. for 1 h and then filtered. The filtrate was added to a solution of spiro[2.5]octan-6-amine hydrogen chloride salt (20 mg, 123.7 μmol) in tetrahydrofuran (2 mL). To this solution were added N,N-dimethylpyridin-4-amine (30 mg, 246.5 μmol) and TEA (123 mg, 1.2 mmol). The mixture was stirred at 40° C. for 2 h. The mixture was concentrated under vacuum. The residue was purified by Prep-HPLC and the collected fractions were lyophilized to afford (R)-2-chloro-8-methyl-N-(spiro[2.5]octan-6-yl)-8-(trifluoromethyl)-7,8-dihydro-6H-pyrazolo[1,5-a]pyrrolo[2,3-e]pyrimidine-6-carboxamide (13.7 mg, 25% yield) as an off-white solid. The enantiomer of Example 172 can be prepared analogously using Method M1 isomer 1.

Example 172: ¹H NMR (400 MHz, DMSO-d₆) δ: 9.30 (s, 1H), 6.99 (s, 1H), 6.87 (d, J=7.6 Hz, 1H), 4.59 (d, J=12.0 Hz, 1H), 3.97 (d, J=12.0 Hz, 1H), 3.54-3.65 (m, 1H), 1.92 (s, 3H), 1.68-1.85 (m, 4H), 1.42-1.51 (m, 2H), 0.88-0.99 (m, 2H), 0.20-0.36 (m, 4H). LC-MS: m/z 428 [M+H]⁺.

### Method U5

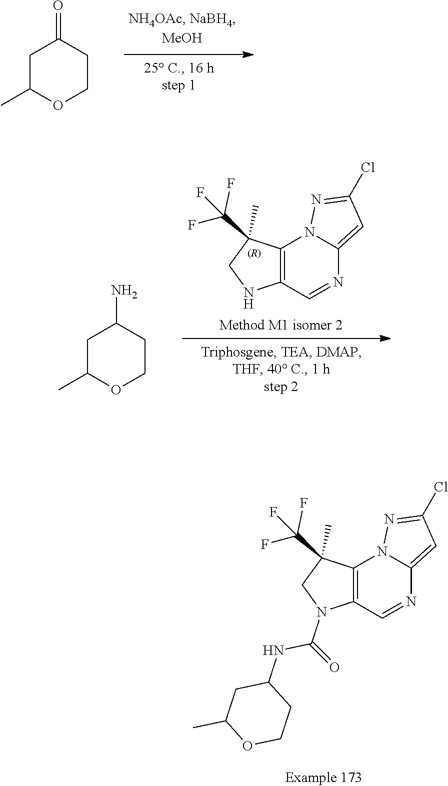

### Examples 173: (8R)-2-chloro-8-methyl-N-(2-methyltetrahydro-2H-pyran-4-yl)-8-(trifluoromethyl)-7,8-dihydro-6H-pyrazolo[1,5-a]pyrrolo[2,3-e]pyrimidine-6-carboxamide

### Step 1: 2-methyltetrahydro-2H-pyran-4-amine

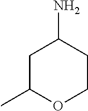

To a stirred solution of 2-methyltetrahydro-4H-pyran-4-one (2.0 g, 17.5 mmol) in methanol (94 mL) was added ammonium acetate (13.5 g, 175.0 mmol). The reaction mixture was stirred at 25° C. for 0.5 h. To this solution was then added NaBH₄ (13.5 g, 19.3 mmol). The reaction mixture was stirred at 25° C. for 16 h. The reaction mixture was quenched with water (500 mL). The resulting solution was extracted with dichloromethane (3×300 mL). The combined organic layers were dried over anhydrous sodium sulfate and concentrated under vacuum. The combined organic layers were concentrated under vacuum to give 2-methyltetrahydro-2H-pyran-4-amine (400 mg, crude) as a yellow oil, which was used directly without further purification. LC-MS: m/z 116 [M+H]⁺.

### Step 2: (8R)-2-chloro-8-methyl-N-(2-methyltetrahydro-2H-pyran-4-yl)-8-(trifluoromethyl)-7,8-dihydro-6H-pyrazolo[1,5-a]pyrrolo[2,3-e]pyrimidine-6-carboxamide

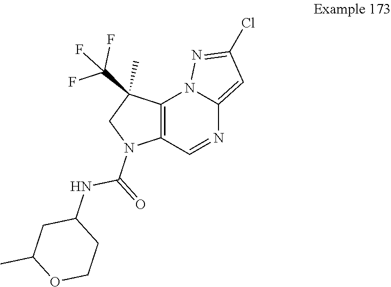 [SMILES: CC1CC(NC(=O)N2C[C@@](C)(C(F)(F)F)c3c2cnc2cc(Cl)nn32)CCO1]

To a stirred solution of Method M1 isomer 2 (50 mg, 181.1 μmol) in tetrahydrofuran (10 mL) were added triphosgene (33 mg, 108.7 μmol) and TEA (28 mg, 271.7 μmol) at 25° C. The resulting mixture was stirred at 28° C. for 0.5 h and then filtered. The resulting filtrate was added to a solution of 2-methyltetrahydro-2H-pyran-4-amine (300 mg, 2.6 mmol) in tetrahydrofuran (1 mL). To this solution were then added TEA (183 mg, 1.8 mmol) and N, N-dimethylpyridin-4-amine (44 mg, 362.3 μmol). The reaction mixture was stirred at 40° C. for 1 h. The mixture was cooled to 25° C. The reaction mixture was quenched with water (50 mL). The resulting solution was extracted with ethyl acetate (3×50 mL). The combined organic layers were dried over anhydrous sodium sulfate and concentrated under vacuum. The residue was purified by column chromatography on silica gel using 80% petroleum ether and 20% ethyl acetate as eluents to afford the crude product which was submitted to Prep-HPLC purification and the collected fractions were lyophilized to give (8R)-2-chloro-8-methyl-N-(2-methyltetrahydro-2H-pyran-4-yl)-8-(trifluoromethyl)-7,8-dihydro-6H-pyrazolo[1,5-a]pyrrolo[2,3-e]pyrimidine-6-carboxamide (3 mg, 4% yield) as a white solid. The stereoisomers of Example 173 can be prepared analogously using Method M1 isomer 1.

Example 173: ¹H NMR (400 MHz, DMSO-d₆) δ: 9.29 (s, 1H), 6.99 (s, 1H), 6.93 (br, 1H), 4.56 (d, J=11.6 Hz, 1H), 3.98 (d, J=12.0 Hz, 1H), 3.86-3.87 (m, 1H), 3.75-3.78 (m, 1H), 3.40-3.44 (m, 2H), 1.92 (s, 3H), 1.80-1.85 (m, 2H), 1.38-1.48 (m, 1H), 1.05-1.22 (m, 4H). LC-MS: m/z 418 [M+H]⁺.

### Method V5

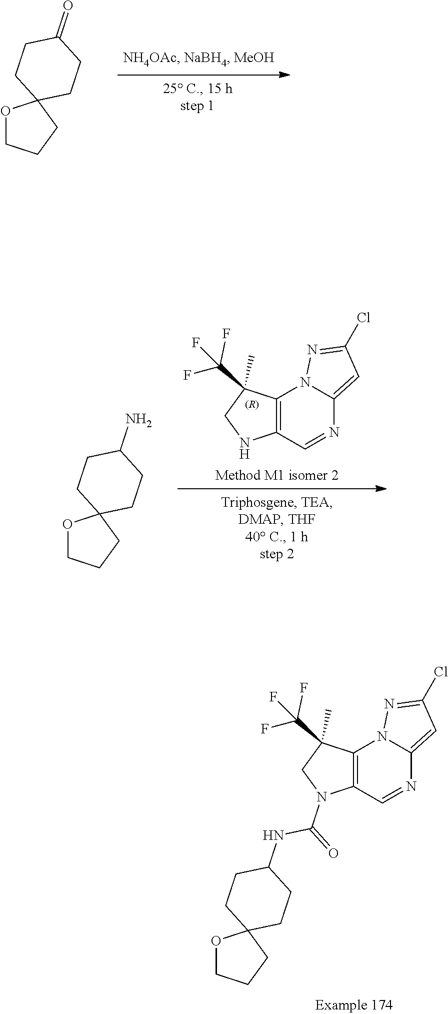

### Example 174: (R)-2-chloro-8-methyl-N-(1-oxaspiro[4.5]decan-8-yl)-8-(trifluoromethyl)-7,8-dihydro-6H-pyrazolo[1,5-a]pyrrolo[2,3-e]pyrimidine-6-carboxamide

### Step 1: 1-oxaspiro[4.5]decan-8-amine

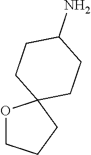 [SMILES: NC1CCC2(CCCO2)CC1]

To a stirred mixture of 1-oxaspiro[4.5]decan-8-one (500 mg, 3.2 mmol) in methanol (2 mL) was added ammonium acetate (2.50 g, 32.4 mmol) at 25° C. The reaction mixture was stirred at 25° C. for 0.5 h. To the mixture was added NaBH₄ (135 mg, 3.6 mmol) in batches at 0° C. The reaction mixture was stirred at 25° C. for 15 h. The reaction mixture was concentrated. The residue was purified by Prep-HPLC and the collected fractions were combined and concentrated under vacuum to afford 1-oxaspiro[4.5]decan-8-amine (60 mg, 12% yield) as yellow oil. LC-MS: m/z 156 [M+H]⁺.

### Step 2: (R)-2-chloro-8-methyl-N-(1-oxaspiro[4.5]decan-8-yl)-8-(trifluoromethyl)-7,8-dihydro-6H-pyrazolo[1,5-a]pyrrolo[2,3-e]pyrimidine-6-carboxamide

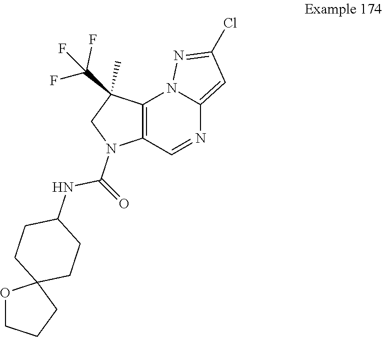 [SMILES: C[C@@]1(C(F)(F)F)CN(C(=O)NC2CCC3(CCCO3)CC2)c2cnc3cc(Cl)nn3c21]

To a stirred solution of Method M1 isomer 2 (50 mg, 180.7 μmol) in tetrahydrofuran (3 mL) were added triphosgene (32 mg, 108.4 μmol) and TEA (27 mg, 271.1 μmol) at 25° C. The resulting mixture was stirred at 40° C. for 0.5 h and then filtered. The resulting filtrate was added to a solution of 1-oxaspiro[4.5]decan-8-amine (28 mg, 180.7 μmol) in tetrahydrofuran (1 mL). To this solution were then added TEA (183 mg, 1.8 mmol) and N,N-dimethylpyridin-4-amine (44 mg, 361.5 μmol). The mixture was stirred at 40° C. for 1 h and concentrated under vacuum. The residue was purified by Prep-HPLC purification and the collected fractions were lyophilized to give (R)-2-chloro-8-methyl-N-(1-oxaspiro[4.5]decan-8-yl)-8-(trifluoromethyl)-7,8-dihydro-6H-pyrazolo[1,5-a]pyrrolo[2,3-e]pyrimidine-6-carboxamide (14.9 mg, 18% yield) as a white solid. The enantiomer of Example 174 can be prepared analogously using Method M1 isomer 1.

Example 174: ¹H NMR (400 MHz, DMSO-d₆) δ: 9.29 (s, 1H), 6.98 (s, 1H), 6.87 (d, J=7.6 Hz, 1H), 4.58 (d, J=11.6 Hz, 1H), 3.95 (d, J=11.6 Hz, 1H), 3.71 (t, J=6.4 Hz, 2H), 3.50-3.54 (m, 1H), 1.91 (s, 3H), 1.81-1.89 (m, 2H), 1.62-1.70 (m, 8H), 1.51-1.55 (m, 2H). LC-MS: m/z 458 [M+H]⁺.

### Method W5

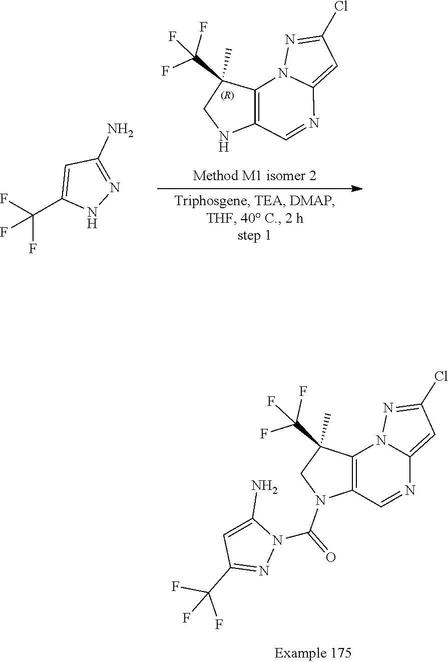

### Example 175: (R)-(5-amino-3-(trifluoromethyl)-1H-pyrazol-1-yl)(2-chloro-8-methyl-8-(trifluoromethyl)-7,8-dihydro-6H-pyrazolo[1,5-a]pyrrolo[2,3-e]pyrimidin-6-yl)methanone

### Step 1: (R)-(5-amino-3-(trifluoromethyl)-1H-pyrazol-1-yl)(2-chloro-8-methyl-8-(trifluoromethyl)-7,8-dihydro-6H-pyrazolo[1,5-a]pyrrolo[2,3-e]pyrimidin-6-yl)methanone

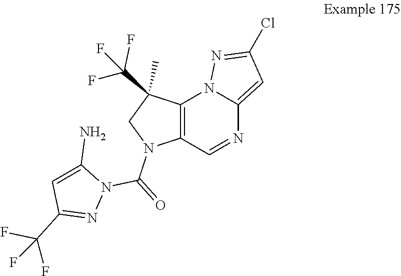 [SMILES: C[C@@]1(C(F)(F)F)CN(C(=O)n2nc(C(F)(F)F)cc2N)c2cnc3cc(Cl)nn3c21]

To a stirred solution of Method M1 isomer 2 (183 mg, 661.8 μmol) in tetrahydrofuran (5 mL) were added triphosgene (118 mg, 397.1 μmol) and TEA (100 mg, 992.8 μmol). The resulting mixture was stirred at 25° C. for 1 h and then filtered. The filtrate was added to a solution of 5-(trifluoromethyl)-1H-pyrazol-3-amine (100 mg, 661.8 μmol) in tetrahydrofuran (2 mL). To this solution were added N,N-dimethylpyridin-4-amine (162 mg, 1.3 mmol) and TEA (670 mg, 6.6 mmol). The mixture was stirred at 40° C. for 2 h. The mixture was concentrated under vacuum. The residue was purified by Prep-HPLC and the collected fractions were lyophilized to afford (R)-(5-amino-3-(trifluoromethyl)-1H-pyrazol-1-yl)(2-chloro-8-methyl-8-(trifluoromethyl)-7,8-dihydro-6H-pyrazolo[1,5-a]pyrrolo[2,3-e]pyrimidin-6-yl)methanone (23.4 mg, 8% yield) as a white solid. The enantiomer of Example 175 can be prepared analogously using Method M1 isomer 1.

Example 175: H NMR (400 MHz, DMSO-d₆) δ: 9.15 (s, 1H), 7.14 (s, 1H), 6.82 (br, 2H), 5.74 (s, 1H), 5.00 (d, J=12.8 Hz, 1H), 4.50 (d, J=12.8 Hz, 1H), 1.95 (s, 3H). LC-MS: m/z 454 [M+H]⁺.

### Method X5

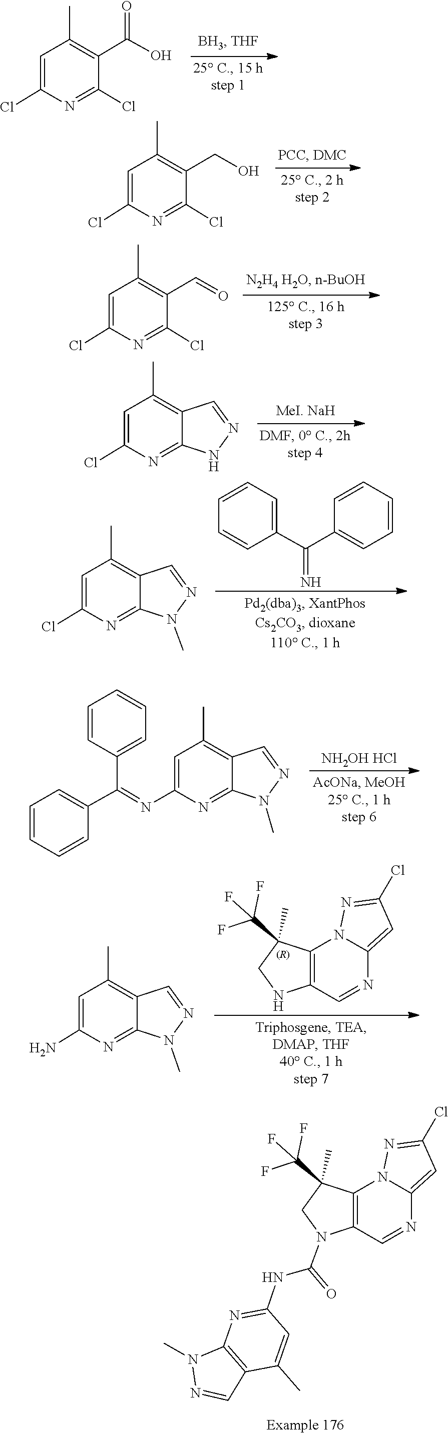

### Example 176: (R)-2-chloro-N-(1,4-dimethyl-1H-pyrazolo[3,4-b]pyridin-6-yl)-8-methyl-8-(trifluoromethyl)-7,8-dihydro-6H-pyrazolo[1,5-a]pyrrolo[2,3-e]pyrimidine-6-carboxamide

### Step 1: (2,6-dichloro-4-methylpyridin-3-yl)methanol

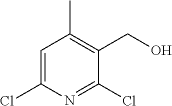

To a stirred solution of 2,6-dichloro-4-methylnicotinic acid (5.0 g, 24.3 mmol) in tetrahydrofuran (50 mL) was added borane (44.9 mL, 44.9 mmol, 1 M in tetrahydrofuran) at 0° C. The reaction mixture was stirred at 25° C. for 15 h. The reaction mixture was quenched with saturated aqueous NaHCO₃ solution (200 mL). The resulting solution was extracted with dichloromethane (3×200 mL). The combined organic layers were dried over anhydrous sodium sulfate and concentrated under vacuum. The residue was purified by column chromatography on silica gel using 95% dichloromethane and 5% methanol as eluents to afford (2,6-dichloro-4-methylpyridin-3-yl)methanol (4.4 g, 84% yield) as a white solid. ¹H NMR (300 MHz, Chloroform-d) δ: 7.15 (s, 1H), 4.83 (s, 2H), 2.50 (s, 3H), 2.08 (br, 1H). LC-MS: m/z 192 [M+H]⁺.

### Step 2: 2,6-dichloro-4-methylnicotinaldehyde

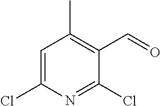

To a stirred solution of (2,6-dichloro-4-methylpyridin-3-yl)methanol (4.2 g, 21.9 mmol) in dichloromethane (150 mL) were added PCC (14.1 g, 65.6 mmol) and silica gel (14.0 g). The reaction mixture was stirred at 25° C. for 2 h. The solid was filtered out. The filtrate was concentrated under vacuum to give 2,6-dichloro-4-methylnicotinaldehyde (3.7 g, 85% yield) as a white solid. ¹H NMR (400 MHz, Chloroform-d) δ: 10.53 (br, 1H), 7.22 (s, 1H), 2.61 (s, 3H). LC-MS: m/z 190 [M+H]⁺

### Step 3: 6-chloro-4-methyl-1H-pyrazolo[3,4-b]pyridine

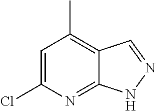

To a stirred solution of 2,6-dichloro-4-methylnicotinaldehyde (3.5 g, 18.4 mmol) in butan-1-ol (60 mL) was added hydrazine hydrate (3.4 g, 55.3 mmol) at 25° C. The reaction mixture was stirred at 125° C. for 16 h. The mixture was cooled to 25° C. The solvent was removed under vacuum. The residue was purified by column chromatography on silica gel using 98% dichloromethane and 2% methanol as eluents to afford 6-chloro-4-methyl-1H-pyrazolo[3,4-b]pyridine (1.7 g, 49% yield) as a white solid. ¹H NMR (300 MHz, DMSO-d₆) δ: 13.73 (br, 1H), 8.24 (s, 1H), 7.08 (s, 1H), 2.58 (s, 3H). LC-MS: m/z 168 [M+H]⁺

### Step 4: 6-chloro-1,4-dimethyl-1H-pyrazolo[3,4-b]pyridine

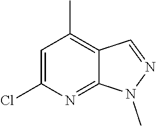

To a stirred solution of 6-chloro-4-methyl-1H-pyrazolo[3,4-b]pyridine (1.6 g, 9.6 mmol) in N,N-dimethylformamide (20 mL) was added NaH (572 mg, 14.3 mmol, 60% in mineral oil) in batches at 0° C. The reaction mixture was stirred at 25° C. for 0.5 h. The iodomethane (2.0 g, 14.3 mmol) was added dropwise at 0° C. The reaction mixture was stirred at 0° C. for 2 h. The reaction mixture was quenched with water (200 mL). The resulting solution was extracted with ethyl acetate (3×200 mL). The combined organic layers were washed with brine (3×500 mL), dried over anhydrous sodium sulfate and concentrated under vacuum. The residue was purified by column chromatography on silica gel using 75% petroleum ether and 25% ethyl acetate as eluents to afford 6-chloro-1,4-dimethyl-1H-pyrazolo[3,4-b]pyridine (1.2 g, 66% yield) as a white solid. ¹H NMR (300 MHz, DMSO-d₆) δ: 8.20 (s, 1H), 7.05 (s, 1H), 3.96 (s, 3H), 2.55 (s, 3H). LC-MS: m/z 182 [M+H]⁺.

### Step 5: N-(1,4-dimethyl-1H-pyrazolo[3,4-b]pyridin-6-yl)-1,1-diphenylmethanimine

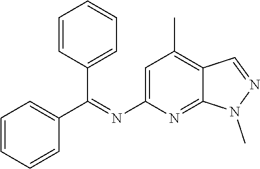 [SMILES: Cc1cc(N=C(c2ccccc2)c2ccccc2)nc2c1cnn2C]

To a stirred solution of 6-chloro-1,4-dimethyl-1H-pyrazolo[3,4-b]pyridine (200 mg, 1.1 mmol) in dioxane (10 mL) were added diphenylmethanimine (399 mg, 2.2 mmol), Xantphos (191 mg, 330.4 μmol), Cs₂CO₃ (1.1 g, 3.3 mmol) and Pd₂(dba)₃ (302 mg, 330.4 μmol) under nitrogen atmosphere. The resulting mixture was stirred at 110° C. for 1 h. The reaction mixture was cooled to 25° C. The solvent was removed under vacuum. The residue was purified by column chromatography on silica gel using 90% petroleum ether and 10% ethyl acetate as eluents to afford N-(1,4-dimethyl-1H-pyrazolo[3,4-b]pyridin-6-yl)-1,1-diphenylmethanimine (320 mg, 62% yield) as a white solid. LC-MS: m/z 327 [M+H]⁺.

### Step 6: 1,4-dimethyl-1H-pyrazolo[3,4-b]pyridin-6-amine

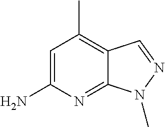

To a stirred solution of N-(1,4-dimethyl-1H-pyrazolo[3,4-b]pyridin-6-yl)-1,1-diphenylmethanimine (320 mg, 980.4 μmol) in methanol (6 mL) were added hydroxylamine hydrochloride (136 mg, 2.0 mmol) and sodium acetate (201 mg, 2.5 mmol). The reaction mixture was stirred at 25° C. for 1 h. The solvent was removed under vacuum. The residue was purified by column chromatography on silica gel using 90% dichloromethane and 10% methanol as eluents to afford 1,4-dimethyl-1H-pyrazolo[3,4-b]pyridin-6-amine (160 mg, 96% yield) as a white solid. ¹H NMR (400 MHz, DMSO-d₆) δ: 7.73 (s, 1H), 6.29 (br, 2H), 6.12 (s, 1H), 3.77 (s, 3H), 2.33 (s, 3H). LC-MS: m/z 163 [M+H]⁺.

### Step 7: (R)-2-chloro-N-(1,4-dimethyl-1H-pyrazolo[3,4-b]pyridin-6-yl)-8-methyl-8-(trifluoromethyl)-7,8-dihydro-6H-pyrazolo[1,5-a]pyrrolo[2,3-e]pyrimidine-6-carboxamide

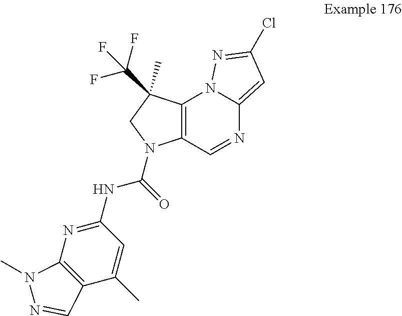 [SMILES: Cc1cc(NC(=O)N2C[C@@](C)(C(F)(F)F)c3c2cnc2cc(Cl)nn32)nc2c1cnn2C]

To a stirred solution of 1,4-dimethyl-1H-pyrazolo[3,4-b]pyridin-6-amine (50 mg, 308.3 μmol) in tetrahydrofuran (5 mL) were added triphosgene (55 mg, 185.0 μmol) and TEA (47 mg, 462.4 μmol). The resulting mixture was stirred at 25° C. for 0.5 h and then filtered. The filtrate was added to a solution of Method M1 isomer 2 (60 mg, 215.8 μmol) in tetrahydrofuran (2 mL). To this solution were added N,N-dimethylpyridin-4-amine (75 mg, 616.6 μmol) and TEA (312 mg, 3.1 mmol). The mixture was stirred at 40° C. for 1 h. The mixture was concentrated under vacuum. The residue was purified by Prep-HPLC and the collected fractions were lyophilized to afford (R)-2-chloro-N-(1,4-dimethyl-1H-pyrazolo[3,4-b]pyridin-6-yl)-8-methyl-8-(trifluoromethyl)-7,8-dihydro-6H-pyrazolo[1,5-a]pyrrolo[2,3-e]pyrimidine-6-carboxamide (30 mg, 21% yield) as a white solid. The enantiomer of Example 176 can be prepared analogously using Method M1 isomer 1.

Example 176: ¹H NMR (400 MHz, DMSO-d₆) δ: 9.95 (br, 1H), 9.33 (s, 1H), 8.12 (s, 1H), 7.59 (s, 1H), 7.05 (s, 1H), 5.04 (d, J=11.6 Hz, 1H), 4.28 (d, J=11.6 Hz, 1H), 3.99 (s, 3H), 2.58 (s, 3H), 1.95 (s, 3H). LC-MS: m/z 465 [M+H]⁺.

### Method Y5

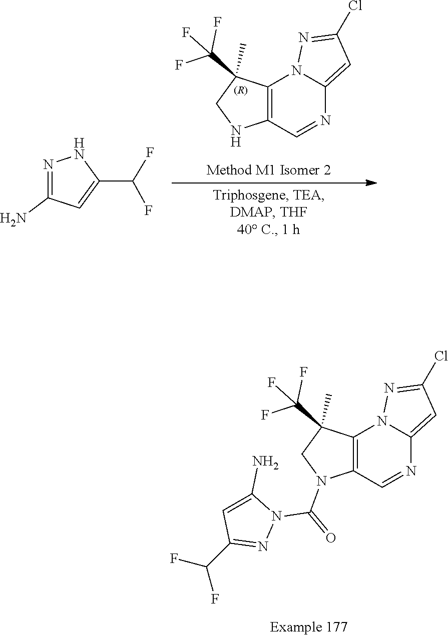

### Examples 177: (R)-(3-amino-5-(difluoromethyl)-1H-pyrazol-1-yl)(2-chloro-8-methyl-8-(trifluoromethyl)-7,8-dihydro-6H-pyrazolo[1,5-a]pyrrolo[2,3-e]pyrimidin-6-yl)methanone

### Step 1: (R)-(3-amino-5-(difluoromethyl)-1H-pyrazol-1-yl)(2-chloro-8-methyl-8-(trifluoromethyl)-7,8-dihydro-6H-pyrazolo[1,5-a]pyrrolo[2,3-e]pyrimidin-6-yl)methanone

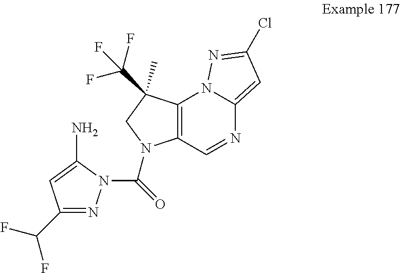 [SMILES: C[C@@]1(C(F)(F)F)CN(C(=O)n2nc(C(F)F)cc2N)c2cnc3cc(Cl)nn3c21]

To a stirred solution of Method M1 isomer 2 (50 mg, 181.2 μmol) in tetrahydrofuran (8 mL) were added triphosgene (33 mg, 180.7 μmol) and TEA (28 mg, 271.7 μmol) at 25° C. The resulting mixture was stirred at 25° C. for 0.5 h and then filtered. The resulting filtrate was added to a solution of 5-(difluoromethyl)-1H-pyrazol-3-amine (48 mg, 362.3 μmol) in tetrahydrofuran (1 mL). To this solution were then added TEA (183 mg, 1.8 mmol) and N, N-Dimethylpyridin-4-amine (45 mg, 362.3 μmol). The reaction mixture was stirred at 40° C. for 1 h. The mixture was cooled to 25° C. The reaction mixture was quenched with water (50 mL). The resulting solution was extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate as eluents to afford the crude product which was submitted to Prep-HPLC purification and the collected fractions were lyophilized to give (R)-(3-amino-5-(difluoromethyl)-1H-pyrazol-1-yl)(2-chloro-8-methyl-8-(trifluoromethyl)-7,8-dihydro-6H-pyrazolo[1,5-a]pyrrolo[2,3-e]pyrimidin-6-yl)methanone (12.6 mg, 16% yield) as a yellow solid. The enantiomer of Example 177 can be prepared analogously using Method M1 isomer 1.

Example 177: ¹H NMR (400 MHz, DMSO-d₆) δ: 9.14 (s, 1H), 7.13 (s, 1H), 6.89 (t, J=56.0 Hz, 1H), 6.66 (br, 2H), 5.59 (s, 1H), 5.00 (d, J=12.8 Hz, 1H), 4.52 (d, J=12.8 Hz, 1H), 1.94 (s, 3H). LC-MS: m/z 436 [M+H]⁺.

### Method Z5

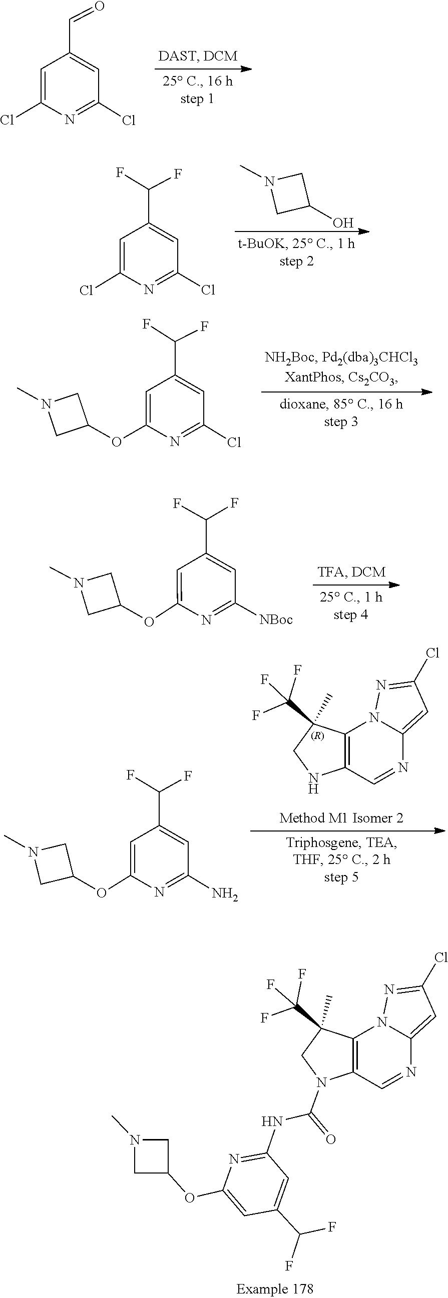

### Examples 178: (R)-2-chloro-N-(4-(difluoromethyl)-6-((1-methylazetidin-3-yl)oxy)pyridin-2-yl)-8-methyl-8-(trifluoromethyl)-7,8-dihydro-6H-pyrazolo[1,5-a]pyrrolo[2,3-e]pyrimidine-6-carboxamide

### Step 1: 2,6-dichloro-4-(difluoromethyl)pyridine

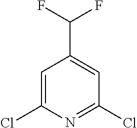 [SMILES: FC(F)c1cc(Cl)nc(Cl)c1]

To a stirred solution of 2,6-dichloropyridine-4-carbaldehyde (10 g, 56.8 mmol) in dichloromethane (500 mL) under nitrogen atmosphere at −78° C. was added DAST (27.5 g, 170.4 mmol). The resulting mixture was warmed to 25° C. The reaction mixture was stirred at 25° C. for 16 h. The reaction mixture was quenched with water (500 mL) and extracted with ethyl acetate (3×500 mL). The combined organic layers were washed with brine (500 mL), dried over anhydrous sodium sulfate and concentrated under vacuum. The residue was purified by column chromatography on silica gel using 80% petroleum ether and 20% ethyl acetate as eluents to afford 2,6-dichloro-4-(difluoromethyl)pyridine (10 g, 86% yield) as a yellow oil. LC-MS: m/z 198 [M+H]⁺.

### Step 2: 2-chloro-4-(difluoromethyl)-6-((1-methylazetidin-3-yl)oxy)pyridine

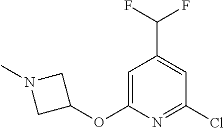 [SMILES: CN1CC(Oc2cc(C(F)F)cc(Cl)n2)C1]

To a stirred solution of 1-methylazetidin-3-ol (3.7 g, 42.1 mmol) in tetrahydrofuran (100 mL) were added 2,6-dichloro-4-(difluoromethyl)pyridine (10 g, 50.5 mmol) and potassium tert-butoxide (9.5 g, 84.2 mmol). The resulting mixture was stirred at 25° C. for 1 h. The reaction mixture was quenched with water (500 mL) and extracted with ethyl acetate (3×500 mL). The combined organic layers were washed with brine (500 mL), dried over anhydrous sodium sulfate and concentrated under vacuum. The residue was purified by column chromatography on silica gel using 75% petroleum ether and 25% ethyl acetate as eluents to afford 2-chloro-4-(difluoromethyl)-6-((1-methylazetidin-3-yl)oxy)pyridine (9.9 g, 73% yield) as a light-yellow oil. LC-MS: m/z 249 [M+H]⁺.

### Step 3: tert-butyl (4-(difluoromethyl)-6-((1-methylazetidin-3-yl)oxy)pyridin-2-yl)carbamate

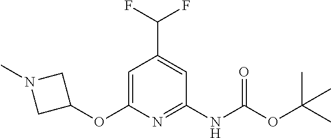 [SMILES: CN1CC(Oc2cc(C(F)F)cc(NC(=O)OC(C)(C)C)n2)C1]

To a stirred solution of 2-chloro-4-(difluoromethyl)-6-(1-methylazetidin-3-yl)oxy-pyridine (560 mg, 2.2 mmol) in dioxane (10 mL) were added tert-butyl carbamate (1.1 g, 9.0 mmol), Pd₂(dba)₃CHCl₃ (233 mg, 225.2 μmol), Xantphos (260 mg, 450.4 μmol) and K₂CO₃ (1.5 g, 4.5 mmol). The reaction mixture was stirred at 85° C. for 16 h under nitrogen atmosphere. The reaction mixture was quenched with water (100 mL), and the resulting solution was extracted with ethyl acetate (3×100 mL). The combined organic layers were washed with brine (200 mL), dried over anhydrous sodium sulfate and concentrated under vacuum. The residue was purified by column chromatography on silica gel using 90% petroleum ether and 10% ethyl acetate as eluents to afford tert-butyl (4-(difluoromethyl)-6-((1-methylazetidin-3-yl)oxy)pyridin-2-yl)carbamate (185 mg, 24% yield) as a yellow oil. LC-MS: m/z 330 [M+H]⁺.

### Step 4: 4-(difluoromethyl)-6-((1-methylazetidin-3-yl)oxy)pyridin-2-amine

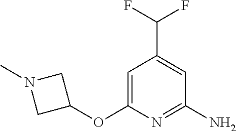 [SMILES: CN1CC(Oc2cc(C(F)F)cc(N)n2)C1]

To a stirred solution tert-butyl (4-(difluoromethyl)-6-((1-methylazetidin-3-yl)oxy)pyridin-2-yl)carbamate (1.3 g, 3.9 mmol) in dichloromethane (30 mL) was added TFA (6 mL). The resulting mixture was stirred at 25° C. for 1 h. The mixture was concentrated under vacuum. The residue was diluted with saturated aqueous NaHCO₃ solution (100 mL). The resulting solution was extracted with dichloromethane (3×100 mL). The combined organic layers were dried over anhydrous sodium sulfate and concentrated under vacuum. The residue was purified by column chromatography on silica gel using 90% dichloromethane and 10% methanol as eluents to afford 4-(difluoromethyl)-6-((1-methylazetidin-3-yl)oxy)pyridin-2-amine (500 mg, 47% yield) as a yellow oil. LC-MS: m/z 230 [M+H]⁺.

### Step 5: (R)-2-chloro-N-(4-(difluoromethyl)-6-((1-methylazetidin-3-yl)oxy)pyridin-2-yl)-8-methyl-8-(trifluoromethyl)-7,8-dihydro-6H-pyrazolo[1,5-a]pyrrolo[2,3-e]pyrimidine-6-carboxamide

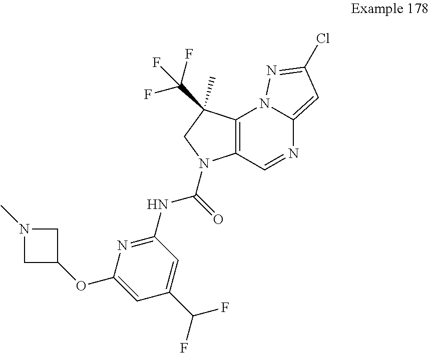 [SMILES: CN1CC(Oc2cc(C(F)F)cc(NC(=O)N3C[C@@](C)(C(F)(F)F)c4c3cnc3cc(Cl)nn43)n2)C1]

To a stirred solution of Method M1 isomer 2 (80 mg, 290.8 μmol) in tetrahydrofuran (2 mL) were added triphosgene (52 mg, 174.5 μmol) and N, N-diethylethanamine (88 mg, 872.5 μmol). The resulting mixture was stirred at 25° C. for 0.5 h and then filtered. The filtrate was added to a solution of 4-(difluoromethyl)-6-((1-methylazetidin-3-yl)oxy)pyridin-2-amine (100 mg, 436.2 μmol) in tetrahydrofuran (1 mL). The mixture was stirred at 25° C. for 2 h. The reaction mixture was quenched with water (50 mL) and extracted with ethyl acetate (3×50 mL). The combined organic layers were washed with brine (50 mL), dried over anhydrous sodium sulfate and concentrated under vacuum. The residue was purified by column chromatography on silica gel using 70% petroleum ether and 30% ethyl acetate as eluents to afford 100 mg of crude product. The afforded crude product was purified by Prep-HPLC purification and the collected fractions were lyophilized to give Example 178 (28 mg, 17% yield) as a yellow solid. The enantiomer of Example 178 can be prepared analogously using Method M1 isomer 1.

Example 178: ¹H NMR (400 MHz, DMSO-d₆) δ: 8.65 (br, 1H), 7.02 (s, 1H), 6.68 (t, J=52.0 Hz, 1H), 6.15 (s, 1H), 5.26-5.29 (m, 2H), 4.32 (d, J=12.0 Hz, 1H), 4.07-4.15 (m, 2H), 3.66-3.84 (m, 3H), 3.07 (s, 3H), 1.86 (s, 3H). LC-MS: m/z 532 [M+H]⁺.

### Method A6

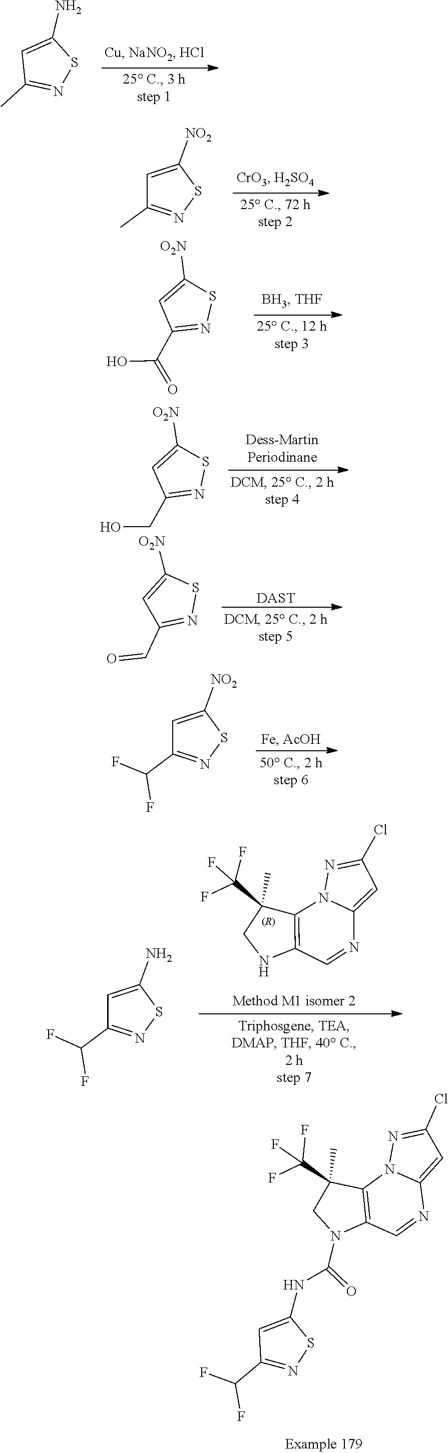

### Example 179: (R)-2-chloro-N-(3-(difluoromethyl)isothiazol-5-yl)-8-methyl-8-(trifluoromethyl)-7,8-dihydro-6H-pyrazolo[1,5-a]pyrrolo[2,3-e]pyrimidine-6-carboxamide

### Step 1: 3-methyl-5-nitroisothiazole

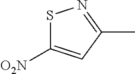

To a stirred mixture of copper (16.7 g, 262.8 mmol) in water (150 mL) were added sodium nitrite (18.1 g, 262.8 mmol) and HCl (0.3 mL, 12 M). The reaction mixture was stirred at 25° C. for 20 min. Then 3-methylisothiazol-5-amine (10 g, 87.6 mmol) in water (100 mL) and HCl (10.9 mL, 12 M) was added dropwise at 25° C. The reaction mixture was stirred at 25° C. for 3 h. The solid was filtered out. The filtrate was extracted with ethyl acetate (3×200 mL). The combined organic layers were dried over anhydrous sodium sulfate and concentrated under vacuum to afford 3-methyl-5-nitroisothiazole (3.4 g, 27% yield) as a red solid. ¹H NMR (300 MHz, DMSO-d₆) δ: 8.11 (s, 1H), 2.50 (s, 3H).

### Step 2: 5-nitroisothiazole-3-carboxylic acid

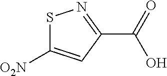 [SMILES: O=C(O)c1cc([N+](=O)[O-])sn1]

To a stirred solution of 3-methyl-5-nitroisothiazole (2.4 g, 16.6 mmol) in sulfuric acid (30 mL) was added chromium(VI) oxide (5.0 g, 49.9 mmol) in portions. The reaction mixture was stirred at 25° C. for 72 h. The reaction mixture was quenched with ice water (200 mL). The resulting mixture was extracted with ethyl acetate (3×100 mL). The combined organic layers were dried over anhydrous sodium sulfate and concentrated under vacuum. The residue was purified by column chromatography on silica gel using 50% petroleum ether and 50% ethyl acetate as eluents to afford 5-nitroisothiazole-3-carboxylic acid (550 mg, 19% yield) as a white solid. LC-MS: m/z 175 [M+H]⁺.

### Step 3: (5-nitroisothiazol-3-yl) methanol

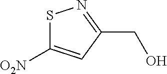

To a stirred solution of 5-nitroisothiazole-3-carboxylic acid (550 mg, 3.2 mmol) in tetrahydrofuran (10 mL) was added borane (4.7 mL, 4.7 mmol, 1 M in tetrahydrofuran). The reaction mixture was stirred at 25° C. for 12 h. The reaction mixture was quenched with methanol (5 mL) at 0° C. The resulting mixture was concentrated under vacuum. The residue was diluted with water (10 mL). The resulting mixture was extracted with ethyl acetate (3×10 mL). The combined organic layers were dried over anhydrous sodium sulfate and concentrated under vacuum. The residue was purified by column chromatography on silica gel using 80% petroleum ether and 20% ethyl acetate as eluents to afford (5-nitroisothiazol-3-yl) methanol (280 mg, 55% yield) as a yellow oil. 1H NMR (300 MHz, DMSO-d₆) δ: 8.09 (s, 1H), 5.75 (t, J=6.3 Hz, 1H), 4.57 (d, J=6.3 Hz, 2H).

### Step 4: 5-nitroisothiazole-3-carbaldehyde

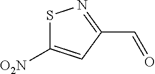

To a stirred solution of (5-nitroisothiazol-3-yl) methanol (300 mg, 1.9 mmol) in dichloromethane (10 mL) was added Dess-Martin Periodinane (953 mg, 2.3 mmol) in portions. The reaction mixture was stirred at 25° C. for 2 h. The reaction mixture was quenched with saturated aqueous NaHCO₃ solution (10 mL). The resulting solution was extracted with dichloromethane (3×10 mL). The combined organic layers were washed with brine (30 mL), dried over anhydrous sodium sulfate and concentrated under vacuum to afford 5-nitroisothiazole-3-carbaldehyde (160 mg, 54% yield) as a yellow oil. LC-MS: m/z 157 [M−H]⁻.

### Step 5: 3-(difluoromethyl)-5-nitroisothiazole

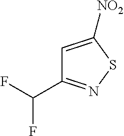 [SMILES: O=[N+]([O-])c1cc(C(F)F)ns1]

To a stirred solution of 5-nitroisothiazole-3-carbaldehyde (300 mg, 1.9 mmol) in dichloromethane (10 mL) was added DAST (917 mg, 5.7 mmol) dropwise at 0° C. The mixture was stirred at 25° C. for 2 h. The reaction mixture was quenched with saturated aqueous NaHCO₃ solution (10 mL). The resulting solution was extracted with dichloromethane (3×10 mL). The combined organic layers were washed with brine (30 mL), dried over anhydrous sodium sulfate and concentrated under vacuum to afford 3-(difluoromethyl)-5-nitroisothiazole (200 mg, 58% yield) as a yellow oil, which was used directly used in next step without further purification. ¹H NMR (300 MHz, DMSO-d₆) δ: 8.54 (s, 1H), 7.16 (t, J=53.7 Hz, 1H).

### Step 6: 3-(difluoromethyl)isothiazol-5-amine

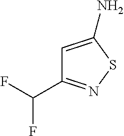

To a stirred mixture of 3-(difluoromethyl)-5-nitroisothiazole (200 mg, 1.1 mmol) in acetic acid (5 mL) was added Fe (186 mg, 3.3 mmol). The mixture was stirred at 50° C. for 2 h. The mixture was diluted with ethyl acetate (10 mL), and the resulting mixture was quenched by 30% ammonium hydroxide solution (20 mL). The resulting mixture was extracted with ethyl acetate (3×20 mL). The combined organic layers were dried over anhydrous sodium sulfate and concentrated under vacuum. The residue was purified by column chromatography on silica gel using 40% petroleum ether and 60% ethyl acetate as eluents to afford 3-(difluoromethyl)isothiazol-5-amine (60 mg, 36% yield) as a yellow oil. ¹H NMR (300 MHz, DMSO-d₆) δ: 6.99 (s, 2H), 6.72 (t, J=54.6 Hz, 1H), 6.31 (s, 1H). LC-MS: m/z 151 [M+H]⁺.

### Step 7: (R)-2-chloro-N-(3-(difluoromethyl)isothiazol-5-yl)-8-methyl-8-(trifluoromethyl)-7,8-dihydro-6H-pyrazolo[1,5-a]pyrrolo[2,3-e]pyrimidine-6-carboxamide

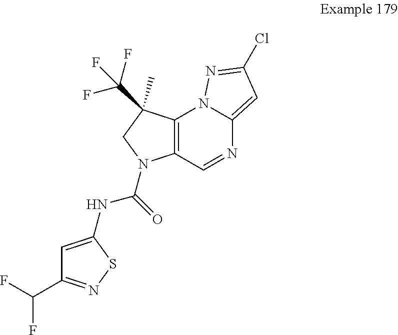 [SMILES: C[C@@]1(C(F)(F)F)CN(C(=O)Nc2cc(C(F)F)ns2)c2cnc3cc(Cl)nn3c21]

To a stirred solution of Method M1 isomer 2 (74 mg, 266.4 μmol) in tetrahydrofuran (2 mL) were added triphosgene (40 mg, 133.2 μmol) and TEA (40 mg, 399.6 μmol). The resulting mixture was stirred at 25° C. for 1 h and then filtered. The filtrate was added to a solution of 3-(difluoromethyl)isothiazol-5-amine (40 mg, 266.4 μmol) in tetrahydrofuran (2 mL). To this solution were added N,N-dimethylpyridin-4-amine (65 mg, 532.8 μmol) and TEA (270 mg, 2.7 mmol). The mixture was stirred at 40° C. for 2 h. The mixture was concentrated under vacuum. The residue was purified by Prep-HPLC and the collected fractions were lyophilized to afford (R)-2-chloro-N-(3-(difluoromethyl)isothiazol-5-yl)-8-methyl-8-(trifluoromethyl)-7,8-dihydro-6H-pyrazolo[1,5-a]pyrrolo[2,3-e]pyrimidine-6-carboxamide (30 mg, 24% yield) as a yellow solid. The enantiomer of Example 179 can be prepared analogously using Method M1 isomer 1.

Example 179: ¹H NMR (300 MHz, DMSO-d₆) δ: 11.34 (s, 1H), 9.34 (s, 1H), 7.12 (s, 1H), 7.08 (s, 1H), 6.97 (t, J=54.6 Hz, 1H), 4.75 (d, J=11.4 Hz, 1H), 4.28 (d, J=11.4 Hz, 1H), 1.96 (s, 3H). LC-MS: m/z 453 [M+H]⁺.

### Method B6

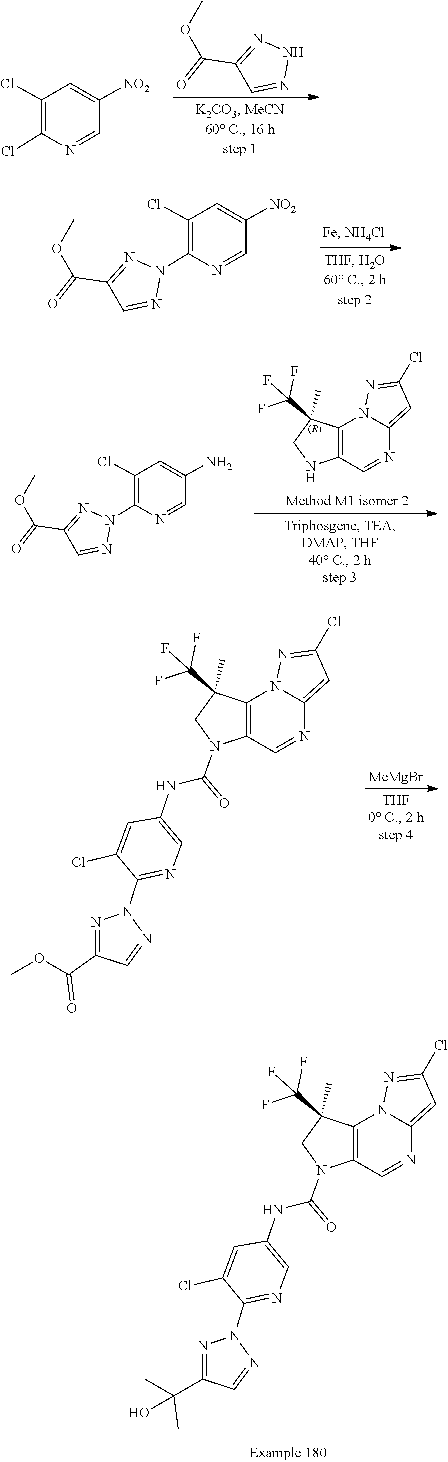

### Example 180: (R)-2-chloro-N-(5-chloro-6-(4-(2-hydroxypropan-2-yl)-2H-1,2,3-triazol-2-yl) pyridine-3-yl)-8-methyl-8-(trifluoromethyl)-7,8-dihydro-6H-pyrazolo[1,5-a]pyrrolo[2,3-e]pyrimidine-6-carboxamide

### Step 1: methyl 2-(3-chloro-5-nitropyridin-2-yl)-2H-1,2,3-triazole-4-carboxylate

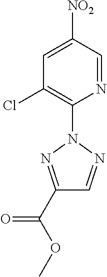 [SMILES: COC(=O)c1cnn(-c2ncc([N+](=O)[O-])cc2Cl)n1]

To a stirred solution of 2,3-dichloro-5-nitropyridine (5 g, 25.9 mmol) in acetonitrile (60 mL) were added potassium carbonate (9.8 g, 70.7 mmol) and methyl 1H-1,2,3-triazole-5-carboxylate (3.0 g, 23.6 mmol). The reaction mixture was stirred at 60° C. for 16 h. The solid was filtered out. The filtrate was concentrated under vacuum. The residue was purified by column chromatography using 80% petroleum ether and 20% ethyl acetate as eluents to afford methyl 2-(3-chloro-5-nitropyridin-2-yl)-2H-1,2,3-triazole-4-carboxylate (4.8 g, 66% yield) as a white solid. LC-MS: m/z 284 [M+H]⁺.

### Step 2: methyl 2-(5-amino-3-chloropyridin-2-yl)-2H-1,2,3-triazole-4-carboxylate

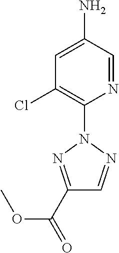 [SMILES: COC(=O)c1cnn(-c2ncc(N)cc2Cl)n1]

To a stirred solution of methyl 2-(3-chloro-5-nitropyridin-2-yl)-2H-1,2,3-triazole-4-carboxylate (3 g, 10.1 mmol) in tetrahydrofuran (20 mL) and water (10 mL) were added Fe (2.8 g, 50.2 mmol) and NH₄Cl (2.7 g, 50.2 mmol). The mixture was stirred at 60° C. for 2 h. After cooled to 25° C., the solid was filtered out. The filtrate was extracted with ethyl acetate (3×20 mL). The combined organic layers were dried over anhydrous sodium sulfate and concentrated under vacuum. The residue was purified by column chromatography on silica gel using 80% petroleum ether and 20% ethyl acetate as eluents to afford methyl 2-(5-amino-3-chloropyridin-2-yl)-2H-1,2,3-triazole-4-carboxylate (1.1 g, 41% yield) as a yellow solid. ¹H NMR (300 MHz, Chloroform-d) δ: 8.30 (s, 1H), 7.92 (d, J=2.7 Hz, 1H), 7.19 (d, J=2.4 Hz, 1H), 3.99 (s, 3H). LC-MS: m/z 254 [M+H]⁺.

### Step 3: methyl (R)-2-(3-chloro-5-(2-chloro-8-methyl-8-(trifluoromethyl)-7,8-dihydro-6H-pyrazolo[1,5-a]pyrrolo[2,3-e]pyrimidine-6-carboxamido)pyridin-2-yl)-2H-1,2,3-triazole-4-carboxylate

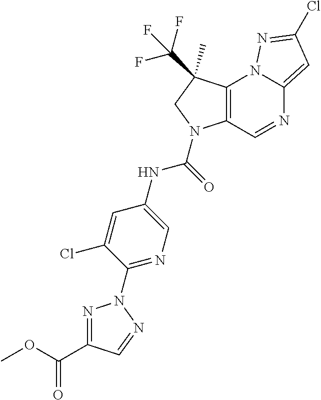 [SMILES: COC(=O)c1cnn(-c2ncc(NC(=O)N3C[C@@](C)(C(F)(F)F)c4c3cnc3cc(Cl)nn43)cc2Cl)n1]

To a stirred solution of methyl 2-(5-amino-3-chloropyridin-2-yl)-2H-1,2,3-triazole-4-carboxylate (55 mg, 216.9 μmol) in tetrahydrofuran (5 mL) were added triphosgene (80 mg, 271.1 μmol) and TEA (28 mg, 271.1 μmol). The resulting mixture was stirred at 25° C. for 1 h and then filtered. The filtrate was added to a solution of Method M1 isomer 2 (50 mg, 180.7 μmol) in tetrahydrofuran (5 mL). To this solution were added N,N-dimethylpyridin-4-amine (44 mg, 361.5 μmol) and TEA (183 mg, 1.8 mmol). The mixture was stirred at 40° C. for 2 h. The reaction mixture was diluted with water (15 mL). The resulting solution was extracted with ethyl acetate (3×15 mL). The combined organic layers were washed with brine (30 mL), dried over anhydrous sodium sulfate and concentrated under vacuum. The residue was purified by column chromatography on silica gel using 90% dichloromethane and 10% methanol as eluents to afford methyl (R)-2-(3-chloro-5-(2-chloro-8-methyl-8-(trifluoromethyl)-7,8-dihydro-6H-pyrazolo[1,5-a]pyrrolo[2,3-e]pyrimidine-6-carboxamido)pyridin-2-yl)-2H-1,2,3-triazole-4-carboxylate (70 mg, 35% yield) as a white solid. LC-MS: m/z 556 [M+H]⁺.

### Step 4: (R)-2-chloro-N-(5-chloro-6-(4-(2-hydroxypropan-2-yl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide

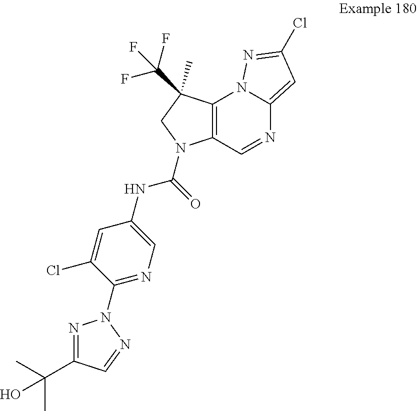 [SMILES: CC(C)(O)c1cnn(-c2ncc(NC(=O)N3C[C@@](C)(C(F)(F)F)c4c3cnc3cc(Cl)nn43)cc2Cl)n1]

To a mixture of methyl (R)-2-(3-chloro-5-(2-chloro-8-methyl-8-(trifluoromethyl)-7,8-dihydro-6H-pyrazolo[1,5-a]pyrrolo[2,3-e]pyrimidine-6-carboxamido)pyridin-2-yl)-2H-1,2,3-triazole-4-carboxylate (30 mg, 53.9 μmol) in tetrahydrofuran (5 mL) was added methylmagnesium bromide (43.1 μL, 129.3 μmol, 3 M in THF) dropwise at 0° C. under nitrogen. The resulting solution was stirred at 0° C. for 2 h. The reaction mixture was quenched with saturated aqueous NH₄Cl solution (2 mL). The resulting mixture was extracted with ethyl acetate (3×5 mL). The combined organic layers were dried over anhydrous sodium sulfate and concentrated under vacuum. The residue was purified by Prep-HPLC and the collected fractions were lyophilized to afford (R)-2-chloro-N-(5-chloro-6-(4-(2-hydroxypropan-2-yl)-2H-1,2,3-triazol-2-yl)pyridin-3-yl)-8-methyl-8-(trifluoromethyl)-7,8-dihydro-6H-pyrazolo[1,5-a]pyrrolo[2,3-e]pyrimidine-6-carboxamide (1.7 mg, 5% yield) as a white solid. The enantiomer of Example 180 can be prepared analogously using Method M1 isomer 1.

Example 180: ¹H NMR (300 MHz, DMSO-d₆) δ: 9.68 (s, 1H), 9.36 (s, 1H), 8.75 (d, J=2.1 Hz, 1H), 8.49 (d, J=2.1 Hz, 1H), 8.32 (s, 1H), 7.08 (s, 1H), 5.26 (s, 1H), 4.85 (d, J=11.4 Hz, 1H), 4.30 (d, J=11.4 Hz, 1H), 1.99 (s, 3H), 1.55 (s, 6H). LC-MS: m/z 556 [M+H]⁺.

### Method C6

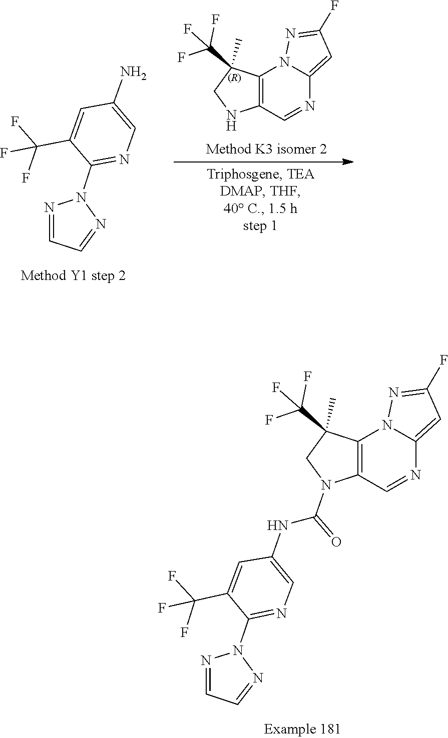

### Example 181: (R)—N-(6-(2H-1,2,3-triazol-2-yl)-5-(trifluoromethyl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

### Step 1: (R)—N-(6-(2H-1,2,3-triazol-2-yl)-5-(trifluoromethyl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

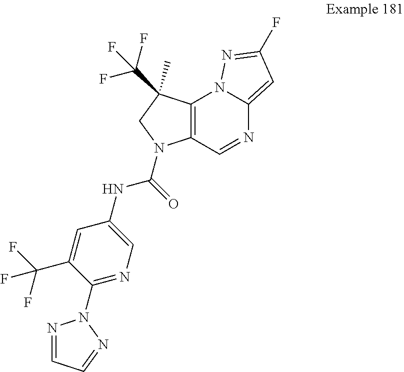 [SMILES: C[C@@]1(C(F)(F)F)CN(C(=O)Nc2cnc(-n3nccn3)c(C(F)(F)F)c2)c2cnc3cc(F)nn3c21]

To a stirred solution of 6-(2H-1,2,3-triazol-2-yl)-5-(trifluoromethyl)pyridin-3-amine (Method Y1 step 2; 26 mg, 115.3 μmol) in tetrahydrofuran (1 mL) were added triphosgene (21 mg, 69.2 μmol) and TEA (23 mg, 226.9 μmol). The resulting mixture was stirred at 25° C. for 0.5 h and then filtered. The filtrate was added to a solution of (R)-2-fluoro-8-methyl-8-(trifluoromethyl)-7,8-dihydro-6H-pyrazolo[1,5-a]pyrrolo[2,3-e]pyrimidine (Method K3 isomer 2; 30 mg, 115.3 μmol) in tetrahydrofuran (1 mL). To this solution were added N,N-dimethylpyridin-4-amine (21 mg, 173.0 μmol) and TEA (57 mg, 567.3 μmol). The mixture was stirred at 40° C. for 1.5 h. The mixture was concentrated under vacuum. The residue was purified by Prep-HPLC and the collected fractions were lyophilized to afford (R)—N-(6-(2H-1,2,3-triazol-2-yl)-5-(trifluoromethyl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide (31.7 mg, 53% yield) as a white solid.

Example 181: ¹H NMR (400 MHz, Chloroform-d) δ: 9.40 (s, 1H), 8.71-8.74 (m, 2H), 7.96 (s, 2H), 7.07 (s, 1H), 6.36 (d, J=5.2 Hz, 1H), 4.62 (d, J=10.4 Hz, 1H), 4.07 (d, J=10.4 Hz, 1H), 2.05 (s, 3H). LC-MS: m/z 516 [M+H]⁺.

### Method D6

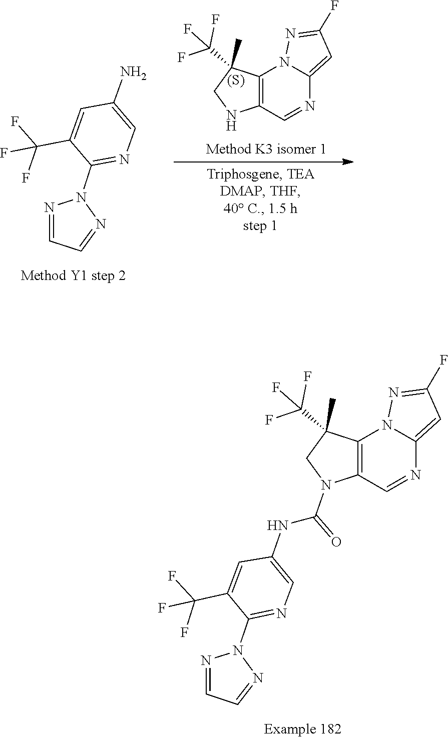

### Example 182: (S)—N-(6-(2H-1,2,3-triazol-2-yl)-5-(trifluoromethyl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

### Step 1: (S)—N-(6-(2H-1,2,3-triazol-2-yl)-5-(trifluoromethyl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

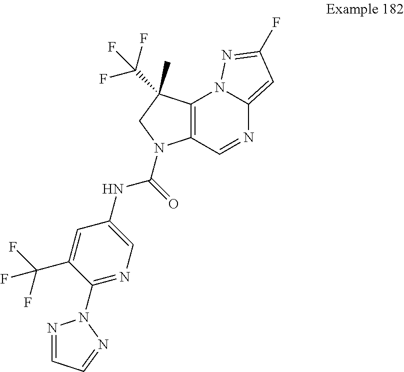 [SMILES: C[C@]1(C(F)(F)F)CN(C(=O)Nc2cnc(-n3nccn3)c(C(F)(F)F)c2)c2cnc3cc(F)nn3c21]

To a stirred solution of 6-(2H-1,2,3-triazol-2-yl)-5-(trifluoromethyl)pyridin-3-amine (Method Y1 step 2; 26 mg, 115.3 μmol) in tetrahydrofuran (1 mL) were added triphosgene (21 mg, 69.2 μmol) and TEA (23 mg, 226.9 μmol). The resulting mixture was stirred at 25° C. for 0.5 h and then filtered. The filtrate was added to a solution of (S)-2-fluoro-8-methyl-8-(trifluoromethyl)-7,8-dihydro-6H-pyrazolo[1,5-a]pyrrolo[2,3-e]pyrimiidine (Method K3 isomer 1; 30 mg, 115.3 μmol) in tetrahydrofuran (1 mL). To this solution were added N,N-dimethylpyridin-4-amine (21 mg, 173.0 μmol) and TEA (57 mg, 567.3 μmol). The mixture was stirred at 40° C. for 1.5 h. The mixture was concentrated under vacuum. The residue was purified by Prep-HPLC and the collected fractions were lyophilized to afford (S)—N-(6-(2H-1,2,3-triazol-2-yl)-5-(trifluoromethyl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide (32.3 mg, 54% yield) as a white solid.

Example 182: ¹HNMR (400 MHz, Chloroform-d) δ: 9.40 (s, 1H), 8.69-8.73 (m, 2H), 7.96 (s, 2H), 7.21 (s, 1H), 6.36 (d, J=5.2 Hz, 1H), 4.62 (d, J=10.4 Hz, 1H), 4.06 (d, J=10.4 Hz, 1H), 2.04 (s, 3H). LC-MS: m/z 516 [M+H]⁺.

### Method E6

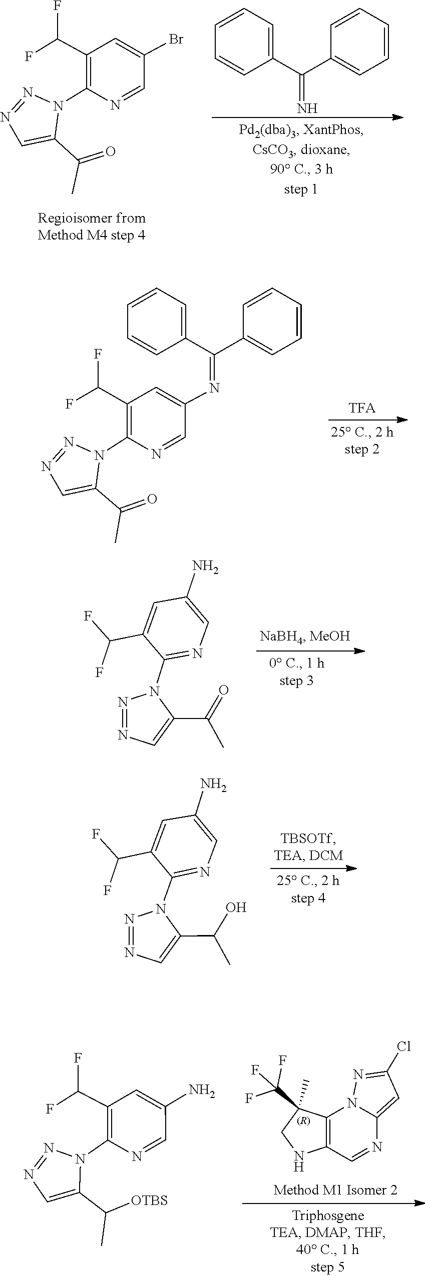

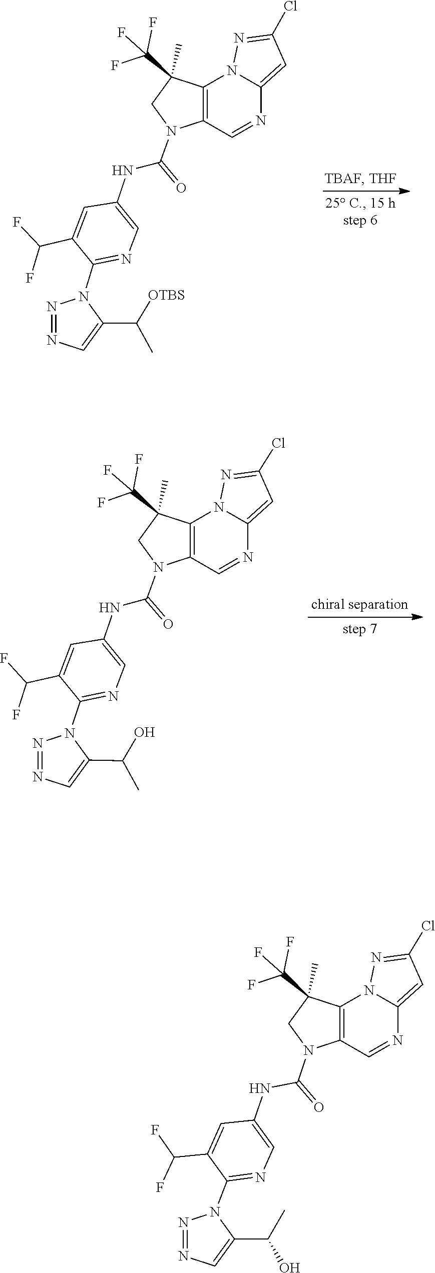 [SMILES: CC(O[Si](C)(C)C(C)(C)C)c1cnnn1-c1ncc(NC(=O)N2C[C@@](C)(C(F)(F)F)c3c2cnc2cc(Cl)nn32)cc1C(F)F ; CC(O)c1cnnn1-c1ncc(NC(=O)N2C[C@@](C)(C(F)(F)F)c3c2cnc2cc(Cl)nn32)cc1C(F)F ; C[C@H](O)c1cnnn1-c1ncc(NC(=O)N2C[C@@](C)(C(F)(F)F)c3c2cnc2cc(Cl)nn32)cc1C(F)F]

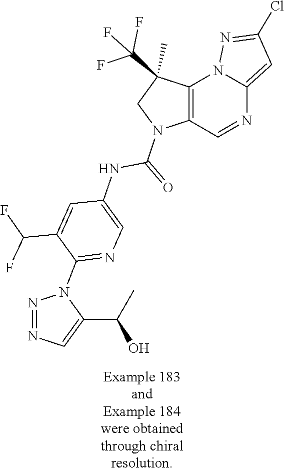 [SMILES: C[C@@H](O)c1cnnn1-c1ncc(NC(=O)N2C[C@@](C)(C(F)(F)F)c3c2cnc2cc(Cl)nn32)cc1C(F)F]

### Examples 183 and 184: Single enantiomers obtained from a racemic mixture containing (R)-2-chloro-N-(5-(difluoromethyl)-6-(5-((R)-1-hydroxyethyl)-1H-1,2,3-triazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(5-(difluoromethyl)-6-(5-((S)-1-hydroxyethyl)-1H-1,2,3-triazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide

### Step 1: 1-(1-(3-(difluoromethyl)-5-((diphenylmethylene)amino)pyridin-2-yl)-1H-1,2,3-triazol-5-yl)ethan-1-one

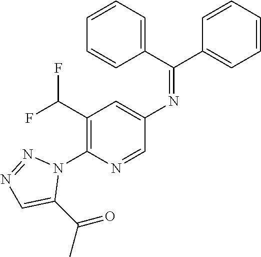 [SMILES: CC(=O)c1cnnn1-c1ncc(N=C(c2ccccc2)c2ccccc2)cc1C(F)F]

To a stirred solution of 1-(1-(5-bromo-3-(difluoromethyl)pyridin-2-yl)-1H-1,2,3-triazol-5-yl)ethan-1-one (Regioisomer from Method M4 step 4; 1.8 g, 5.7 mmol) and diphenylmethanimine (1.1 g, 6.2 mmol) in dioxane (20 mL) were added Pd₂(dba)₃ (259 mg, 283.8 μmol), Cs₂CO₃ (4.6 g, 14.1 mmol) and XantPhos (246 mg, 425.0 μmol) under nitrogen atmosphere. The reaction mixture was stirred at 90° C. for 3 h. After cooled to 25° C., the reaction mixture was filtered. The filter cake was washed with ethyl acetate (10 mL). The filtrate was poured into water (50 mL), and the resulting solution was extracted with ethyl acetate (3×80 mL). The combined organic layers were dried over anhydrous sodium sulfate and concentrated under vacuum. The residue was purified by column chromatography on silica gel using 60% petroleum ether and 40% ethyl acetate as eluents to afford 1-(1-(3-(difluoromethyl)-5-((diphenylmethylene)amino)pyridin-2-yl)-1H-1,2,3-triazol-5-yl)ethan-1-one (850 mg, 36% yield) as a yellow solid. LC-MS: m/z 418 [M+H]⁺.

### Step 2: 1-(1-(5-amino-3-(difluoromethyl)pyridin-2-yl)-1H-1,2,3-triazol-5-yl)ethan-1-one

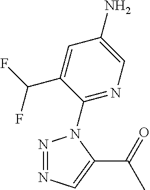 [SMILES: CC(=O)c1cnnn1-c1ncc(N)cc1C(F)F]

A solution of 1-(1-(3-(difluoromethyl)-5-((diphenylmethylene)amino)pyridin-2-yl)-1H-1,2,3-triazol-5-yl)ethan-1-one (700 mg, 1.7 mmol) in TFA (20 mL) was stirred at 25° C. for 2 h. The reaction mixture was concentrated under vacuum. The residue was diluted with saturated aqueous NaHCO₃ solution (40 mL). The resulting solution was extracted with ethyl acetate (3×50 mL). The combined organic layers were dried over anhydrous sodium sulfate and concentrated under vacuum. The residue was purified by column chromatography on silica gel using 70% petroleum ether and 30% ethyl acetate as eluents to afford 1-(1-(5-amino-3-(difluoromethyl)pyridin-2-yl)-1H-1,2,3-triazol-5-yl)ethan-1-one (260 mg, 59% yield) as a yellow solid. LC-MS: m/z 254 [M+H]⁺.

### Step 3: 1-(1-(5-amino-3-(difluoromethyl)pyridin-2-yl)-1H-1,2,3-triazol-5-yl)ethan-1-ol

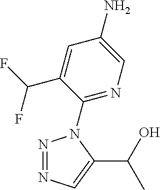 [SMILES: CC(O)c1cnnn1-c1ncc(N)cc1C(F)F]

To a stirred mixture of 1-(1-(5-amino-3-(difluoromethyl)pyridin-2-yl)-1H-1,2,3-triazol-5-yl)ethan-1-one (165 mg, 651.6 μmol) in methanol (30 mL) was added NaBH₄ (30 mg, 782.0 μmol) at 0° C. The resulting mixture was stirred at 0° C. for 1 h. The resulting mixture was concentrated under vacuum. The residue was purified by column chromatography on silica gel using 30% petroleum ether and 70% ethyl acetate as eluents to afford 1-(1-(5-amino-3-(difluoromethyl)pyridin-2-yl)-1H-1,2,3-triazol-5-yl)ethan-1-ol (140 mg, 85% yield) as a white solid. ¹H NMR (300 MHz, DMSO-d₆) δ: 8.28 (s, 1H), 7.98 (d, J=2.7 Hz, 1H), 7.37 (d, J=2.7 Hz, 1H), 7.12 (t, J=53.4 Hz, 1H), 6.12 (br, 2H), 5.34 (d, J=5.7 Hz, 1H), 4.85-4.93 (m, 1H), 1.45 (d, J=6.6 Hz, 3H). LC-MS: m/z 256 [M+H]⁺.

### Step 4: 6-(5-(1-((tert-butyldimethylsilyl)oxy)ethyl)-1H-1,2,3-triazol-1-yl)-5-(difluoromethyl)pyridin-3-amine

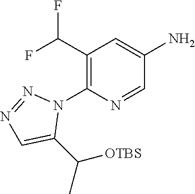 [SMILES: CC(O[Si](C)(C)C(C)(C)C)c1cnnn1-c1ncc(N)cc1C(F)F]

To a stirred solution of 1-(1-(5-amino-3-(difluoromethyl)pyridin-2-yl)-1H-1,2,3-triazol-5-yl)ethan-1-ol (140 mg, 548.5 μmol) in dichloromethane (10 mL) were added tert-butyldimethylsilyl trifluoromethanesulfonate (290 mg, 1.1 mmol) and TEA (167 mg, 1.7 mmol) at 0° C. The reaction mixture was stirred at 25° C. for 2 h. The reaction mixture was concentrated under vacuum. The residue was purified by Prep-TLC using 80% petroleum ether and 20% ethyl acetate as eluents to afford 6-(5-(1-((tert-butyldimethylsilyl)oxy)ethyl)-1H-1,2,3-triazol-1-yl)-5-(difluoromethyl)pyridin-3-amine (170 mg, 79% yield) as a white solid. ¹H NMR (300 MHz, DMSO-d₆) δ: 8.29 (s, 1H), 7.98 (d, J=2.7 Hz, 1H), 7.37 (d, J=2.7 Hz, 1H), 7.10 (t, J=54.6 Hz, 1H), 6.13 (br, 2H), 5.09 (q, J=6.6 Hz, 1H), 1.49 (d, J=6.3 Hz, 3H), 0.85 (s, 9H), 0.09 (s, 3H), 0.01 (s, 3H). LC-MS: m/z 370 [M+H]⁺.

### Step 5: (8R)—N-(6-(5-(1-((tert-butyldimethylsilyl)oxy)ethyl)-1H-1,2,3-triazol-1-yl)-5-(difluoromethyl)pyridin-3-yl)-2-chloro-8-methyl-8-(trifluoromethyl)-7,8-dihydro-6H-pyrazolo[1,5-a]pyrrolo[2,3-e]pyrimidine-6-carboxamide

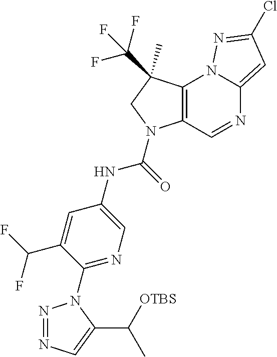 [SMILES: CC(O[Si](C)(C)C(C)(C)C)c1cnnn1-c1ncc(NC(=O)N2C[C@@](C)(C(F)(F)F)c3c2cnc2cc(Cl)nn32)cc1C(F)F]

To a stirred solution of 6-(5-(1-((tert-butyldimethylsilyl)oxy)ethyl)-1H-1,2,3-triazol-1-yl)-5-(difluoromethyl)pyridin-3-amine (90 mg, 243.6 μmol) in tetrahydrofuran (3 mL) were added TEA (37 mg, 365.4 μmol) and triphosgene (43 mg, 146.2 μmol). The resulting mixture was stirred at 25° C. for 0.5 h and then filtered. The filtrate was added to a solution of Method M1 isomer 2 (47 mg, 170.5 μmol) in tetrahydrofuran (1 mL). To this solution were added N,N-dimethylpyridin-4-amine (60 mg, 487.2 μmol) and TEA (246 mg, 2.4 mmol). The reaction mixture was stirred at 40° C. for 1 h. The reaction mixture was concentrated under vacuum. The residue was purified by Prep-TLC using 97% dichloromethane and 3% methanol as eluents to afford (8R)—N-(6-(5-(1-((tert-butyldimethylsilyl)oxy)ethyl)-1H-1,2,3-triazol-1-yl)-5-(difluoromethyl)pyridin-3-yl)-2-chloro-8-methyl-8-(trifluoromethyl)-7,8-dihydro-6H-pyrazolo[1,5-a]pyrrolo[2,3-e]pyrimidine-6-carboxamide (150 mg, 79% yield) as a white solid. LC-MS: m/z 672 [M+H]⁺.

### Step 6: (8R)-2-chloro-N-(5-(difluoromethyl)-6-(5-(1-hydroxyethyl)-1H-1,2,3-triazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide

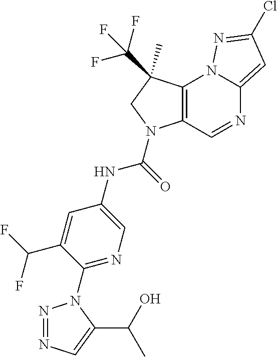 [SMILES: CC(O)c1cnnn1-c1ncc(NC(=O)N2C[C@@](C)(C(F)(F)F)c3c2cnc2cc(Cl)nn32)cc1C(F)F]

To a stirred solution of (8R)—N-(6-(5-(1-((tert-butyldimethylsilyl)oxy)ethyl)-1H-1,2,3-triazol-1-yl)-5-(difluoromethyl)pyridin-3-yl)-2-chloro-8-methyl-8-(trifluoromethyl)-7,8-dihydro-6H-pyrazolo[1,5-a]pyrrolo[2,3-e]pyrimidine-6-carboxamide (150 mg, 223.3 μmol) in tetrahydrofuran (5 mL) was added tetrabutylammonium fluoride (1 mL, 1 M in tetrahydrofuran) at 25° C. The reaction mixture was stirred at 25° C. for 15 h. The reaction mixture was concentrated under vacuum. The residue was purified by Prep-TLC using 93% dichloromethane and 7% methanol as eluents to afford (8R)-2-chloro-N-(5-(difluoromethyl)-6-(5-(1-hydroxyethyl)-1H-1,2,3-triazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide (60 mg, 48% yield) as a white solid. ¹H NMR (300 MHz, DMSO-d₆) δ: 9.67 (s, 1H), 9.35 (s, 1H), 8.96 (d, J=2.1 Hz, 1H), 8.58 (d, J=2.4 Hz, 1H), 8.52 (s, 1H), 7.44 (t, J=54.3 Hz, 1H), 7.06 (s, 1H), 4.89-4.96 (m, 1H), 4.85 (d, J=11.4 Hz, 1H), 4.30 (d, J=11.4 Hz, 1H), 1.98 (s, 3H), 1.48 (d, J=6.6 Hz, 3H). LC-MS: m/z 558 [M+H]⁺.

### Step 7: Separation of enantiomers to obtain (R)-2-chloro-N-(5-(difluoromethyl)-6-(5-((R)-1-hydroxyethyl)-1H-1,2,3-triazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide and (R)-2-chloro-N-(5-(difluoromethyl)-6-(5-((S)-1-hydroxyethyl)-1H-1,2,3-triazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide

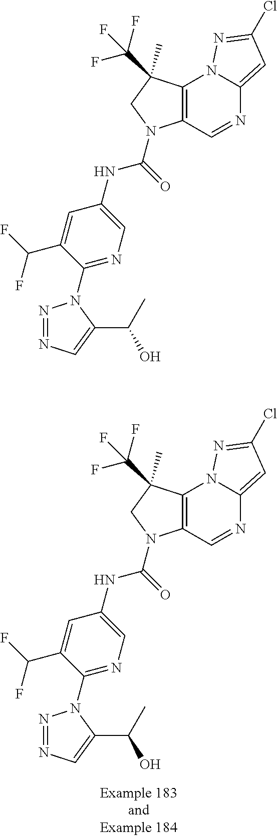 [SMILES: C[C@H](O)c1cnnn1-c1ncc(NC(=O)N2C[C@@](C)(C(F)(F)F)c3c2cnc2cc(Cl)nn32)cc1C(F)F ; C[C@@H](O)c1cnnn1-c1ncc(NC(=O)N2C[C@@](C)(C(F)(F)F)c3c2cnc2cc(Cl)nn32)cc1C(F)F]

(8R)-2-chloro-N-(5-(difluoromethyl)-6-(5-(1-hydroxyethyl)-1H-1,2,3-triazol-1-yl)pyridin-3-yl)-8-methyl-8-(trifluoromethyl)-7,8-dihydro-6H-pyrazolo[1,5-a]pyrrolo[2,3-e]pyrimidine-6-carboxamide (50 mg, 89.6 μmol) was submitted to chiral HPLC purification: Column: CHIRALPAK IA, 3×25 cm, 5 μm; Mobile Phase A: Hex (0.5% 2M NH₃-MeOH)—HPLC, Mobile Phase B: EtOH—HPLC; Flow rate: 40 mL/min; Gradient: 15% B to 15% B in 28 min; Wave Length: 220/254 nm; RT1 (min): 19.3; RT2 (min): 23.7; Sample Solvent: EtOH-HPLC; Injection Volume: 0.5 mL; Number Of Runs: 5. The first eluting isomer was concentrated and lyophilized to afford Example 183 (9.1 mg, 18% yield) as a white solid. The second eluting isomer was concentrated and lyophilized to afford Example 184 (13.2 mg, 26% yield) as a white solid. The corresponding stereoisomers of Examples 183 and 184 can be prepared analogously using Method M1 isomer 1. Examples 183 and 184 are diastereomers, wherein the stereocenter attached to the trifluoromethyl is absolute and the carbinol stereocenter is relative (i.e., the carbinol stereocenter in one of Examples 183 and 184 is (S), and the carbinol stereocenter in the other of Examples 183 and 184 is (R)).

Example 183: ¹H NMR (400 MHz, DMSO-d₆) δ: 9.70 (s, 1H), 9.37 (s, 1H), 8.98 (d, J=2.0 Hz, 1H), 8.60 (d, J=2.0 Hz, 1H), 8.53 (s, 1H), 7.46 (t, J=54.4 Hz, 1H), 7.08 (s, 1H), 5.44 (d, J=4.8 Hz, 1H), 4.92-4.96 (m, 1H), 4.87 (d, J=11.6 Hz, 1H), 4.33 (d, J=11.6 Hz, 1H), 1.99 (s, 3H), 1.49 (d, J=6.8 Hz, 3H). LC-MS: m/z 558 [M+H]⁺.

Example 184: ¹H NMR (300 MHz, DMSO-d₆) δ: 9.67 (s, 1H), 9.35 (s, 1H), 8.97 (d, J=2.4 Hz, 1H), 8.58 (d, J=2.1 Hz, 1H), 8.51 (s, 1H), 7.44 (t, J=54.0 Hz, 1H), 7.06 (s, 1H), 5.40 (d, J=5.1 Hz, 1H), 4.91-4.95 (m, 1H), 4.85 (d, J=11.4 Hz, 1H), 4.30 (d, J=11.4 Hz, 1H), 1.97 (s, 3H), 1.47 (d, J=6.6 Hz, 3H). LC-MS: m/z 558 [M+H]⁺.

### Method F6

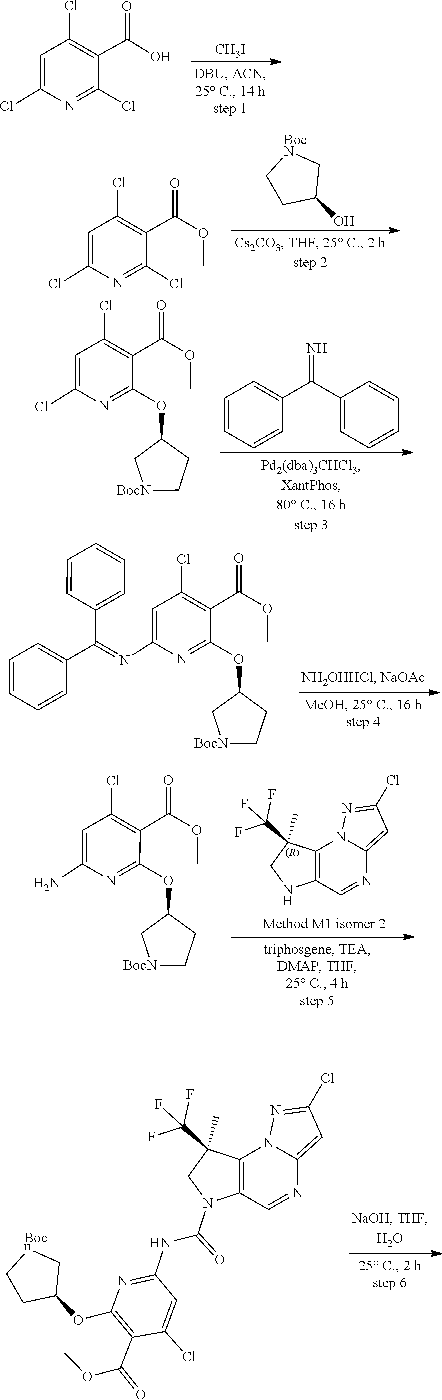

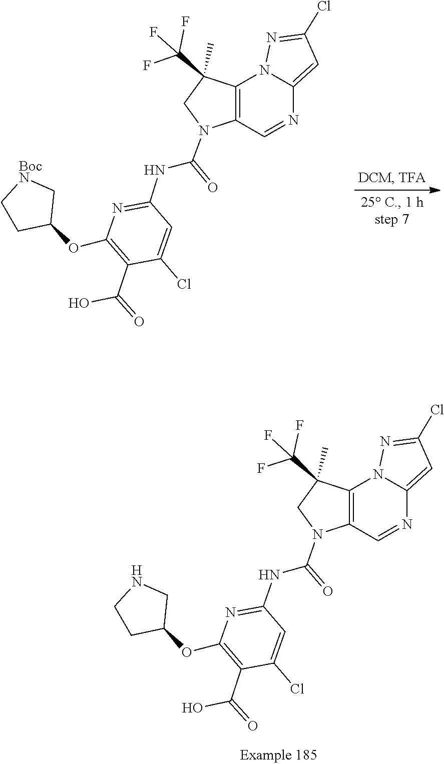 [SMILES: CC(C)(C)OC(=O)N1CC[C@H](Oc2nc(NC(=O)N3C[C@@](C)(C(F)(F)F)c4c3cnc3cc(Cl)nn43)cc(Cl)c2C(=O)O)C1 ; C[C@@]1(C(F)(F)F)CN(C(=O)Nc2cc(Cl)c(C(=O)O)c(O[C@H]3CCNC3)n2)c2cnc3cc(Cl)nn3c21]

### Example 185: 4-chloro-6-((R)-2-chloro-1-methyl-8-(trifluoromethyl)-7,8-dihydro-6H-pyrazolo[1,5-a]pyrrolo[2,3-e]pyrimidine-6-carboxamido)-2-(((S)-pyrrolidin-3-yl)oxy)nicotinic acid

### Step 1: methyl 2,4,6-trichloronicotinate

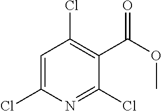

To a stirred mixture of 2,4,6-trichloronicotinic acid (20 g, 88.3 mmol) in acetonitrile (400 mL) were added iodomethane (62.6 g, 441.6 mmol) and DBU (40.3 g, 291.8 mmol) at 0° C. The reaction mixture was stirred at 25° C. for 14 h. The reaction mixture was concentrated under vacuum. The residue was diluted with water (800 mL), and the resulting solution was extracted with ethyl acetate (3×1000 mL). The combined organic layers were dried over anhydrous sodium sulfate and concentrated under vacuum. The residue was purified by column chromatography using 80% petroleum ether and 20% ethyl acetate as eluents to afford methyl 2,4,6-trichloronicotinate (18.6 g, 84% yield) as a yellow oil. LC-MS: m/z 240 [M+H]⁺.

### Step 2: methyl (S)-2-((1-(tert-butoxycarbonyl)pyrrolidin-3-yl)oxy)-4,6-dichloronicotinate

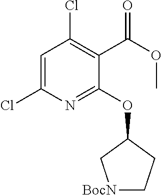 [SMILES: COC(=O)c1c(Cl)cc(Cl)nc1O[C@H]1CCN(C(=O)OC(C)(C)C)C1]

To a stirred mixture of tert-butyl (S)-3-hydroxypyrrolidine-1-carboxylate (7 g, 37.4 mmol) in tetrahydrofuran (200 mL) were added methyl 2,4,6-trichloronicotinate (9 g, 37.4 mmol) and cesium carbonate (24.4 g, 74.8 mmol) at 25° C. The reaction mixture was stirred at 25° C. for 2 h. The solid was filtered out. The filtrate was concentrated under vacuum. The residue was purified by column chromatography using 80% petroleum ether and 20% ethyl acetate as eluents to afford methyl (S)-2-((1-(tert-butoxycarbonyl)pyrrolidin-3-yl)oxy)-4,6-dichloronicotinate (3.9 g, 26% yield) as a yellow oil. LC-MS: m/z 391[M+H]⁺.

### Step 3: methyl (S)-2-((1-(tert-butoxycarbonyl)pyrrolidin-3-yl)oxy)-4-chloro-6-((diphenylmethylene)amino)nicotinate

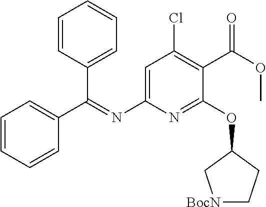 [SMILES: COC(=O)c1c(Cl)cc(N=C(c2ccccc2)c2ccccc2)nc1O[C@H]1CCN(C(=O)OC(C)(C)C)C1]

To a stirred solution of methyl (S)-2-((1-(tert-butoxycarbonyl)pyrrolidin-3-yl)oxy)-4,6-dichloronicotinate (3.9 g, 9.9 mmol) in dioxane (200 mL) were added diphenylmethanimine (2.7 g, 14.9 mmol), Pd₂(dba)₃CHCl₃ (1.0 g, 996.8 μmol), XantPhos (1.1 g, 1.9 mmol), and Cs₂CO₃ (6.5 g, 19.9 mmol). The resulting mixture was stirred at 80° C. for 16 h. The mixture was cooled to 25° C. The mixture was concentrated under vacuum. The residue was diluted with water (200 mL), and resulting solution was extracted with ethyl acetate (3×200 mL). The combined organic layers were washed with brine (200 mL), dried over anhydrous sodium sulfate and concentrated under vacuum. The residue was submitted to Prep-HPLC purification and the collected fractions were lyophilized to give methyl (S)-2-((1-(tert-butoxycarbonyl)pyrrolidin-3-yl)oxy)-4-chloro-6-((diphenylmethylene)amino)nicotinate (3 g, 13% yield) as a yellow oil. LC-MS: m/z 536 [M+H]⁺.

### Step 4: methyl (S)-6-amino-2-((1-(tert-butoxycarbonyl)pyrrolidin-3-yl)oxy)-4-chloronicotinate

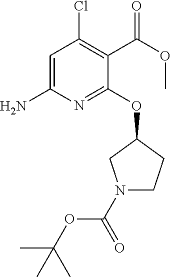 [SMILES: COC(=O)c1c(Cl)cc(N)nc1O[C@H]1CCN(C(=O)OC(C)(C)C)C1]

To a stirred solution of methyl (S)-2-((1-(tert-butoxycarbonyl)pyrrolidin-3-yl)oxy)-4-chloro-6-((diphenylmethylene)amino)nicotinate (3 g, 5.6 mmol) in methanol (100 mL) were added hydroxylamine hydrochloride (583 mg, 8.4 mmol) and sodium acetate (918 mg, 11.1 mmol). The mixture was stirred at 25° C. for 16 h. The mixture was concentrated under vacuum. The residue was diluted with water (200 mL), and the resulting solution was extracted with ethyl acetate (3×200 mL). The combined organic layers were washed with brine (200 mL), dried over anhydrous sodium sulfate and concentrated under vacuum. The residue was purified by column chromatography on silica gel using 75% petroleum ether and 25% ethyl acetate to afford methyl (S)-6-amino-2-((1-(tert-butoxycarbonyl)pyrrolidin-3-yl)oxy)-4-chloronicotinate (1 g, 44% yield) as a yellow oil. LC-MS: m/z 372 [M+H]⁺.

### Step 5: methyl 2-(((S)-1-(tert-butoxycarbonyl)pyrrolidin-3-yl)oxy)-4-chloro-6-((R)-2-chloro-8-methyl-8-(trifluoromethyl)-7,8-dihydro-6H-pyrazolo[1,5-a]pyrrolo[2,3-e]pyrimidine-6-carboxamido)nicotinate

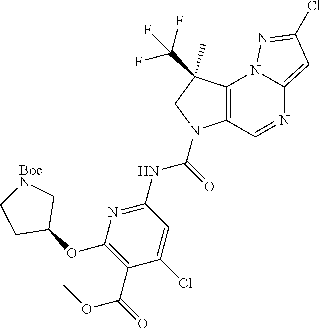 [SMILES: COC(=O)c1c(Cl)cc(NC(=O)N2C[C@@](C)(C(F)(F)F)c3c2cnc2cc(Cl)nn32)nc1O[C@H]1CCN(C(=O)OC(C)(C)C)C1]

To a stirred solution of Method M1 isomer 2 (49 mg, 179.3 μmol) in tetrahydrofuran (2 mL) were added triphosgene (31 mg, 107.5 μmol) and TEA (54 mg, 537.9 μmol). The resulting mixture was stirred at 25° C. for 1 h and then filtered. The filtrate was added to a solution of methyl (S)-6-amino-2-((1-(tert-butoxycarbonyl)pyrrolidin-3-yl)oxy)-4-chloronicotinate (100 mg, 268.9 μmol) in tetrahydrofuran (1 mL). To this solution were added N,N-dimethylpyridin-4-amine (43 mg, 358.6 μmol) and TEA (108 mg, 1.1 mmol). The mixture was stirred at 25° C. for 3 h. The mixture was concentrated under vacuum. The residue was purified by Prep-HPLC and the collected fractions were lyophilized to afford methyl 2-(((S)-1-(tert-butoxycarbonyl)pyrrolidin-3-yl)oxy)-4-chloro-6-((R)-2-chloro-8-methyl-8-(trifluoromethyl)-7,8-dihydro-6H-pyrazolo[1,5-a]pyrrolo[2,3-e]pyrimidine-6-carboxamido)nicotinate (100 mg, 66% yield) as a white solid. LC-MS: m/z 674 [M+H]⁺.

### Step 6: 2-(((S)-1-(tert-butoxycarbonyl)pyrrolidin-3-yl)oxy)-4-chloro-6-((R)-2-chloro-8-methyl-8-(trifluoromethyl)-7,8-dihydro-6H-pyrazolo[1,5-a]pyrrolo[2,3-e]pyrimidine-6-carboxamido)nicotinic acid

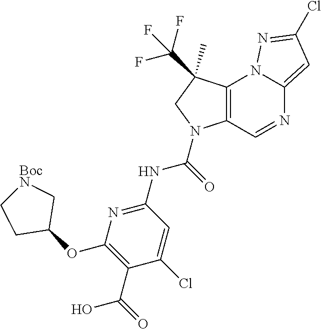 [SMILES: CC(C)(C)OC(=O)N1CC[C@H](Oc2nc(NC(=O)N3C[C@@](C)(C(F)(F)F)c4c3cnc3cc(Cl)nn43)cc(Cl)c2C(=O)O)C1]

To a stirred solution of methyl 2-(((S)-1-(tert-butoxycarbonyl)pyrrolidin-3-yl)oxy)-4-chloro-6-((R)-2-chloro-8-methyl-8-(trifluoromethyl)-7,8-dihydro-6H-pyrazolo[1,5-a]pyrrolo[2,3-e]pyrimidine-6-carboxamido)nicotinate (50 mg, 74 μmol) in tetrahydrofuran (2 mL) was added sodium hydroxide (6 mg, 148.27 μmol) and water (1 mL). The reaction mixture was stirred at 25° C. for 2 h. The pH was adjusted to 3 with HCl (1 M). The resulting solution was extracted with ethyl acetate (3×50 mL). The combined organic layers were washed with brine (100 mL), dried over anhydrous sodium sulfate and concentrated under vacuum. The reaction mixture was concentrated to afford 2-(((S)-1-(tert-butoxycarbonyl)pyrrolidin-3-yl)oxy)-4-chloro-6-((R)-2-chloro-8-methyl-8-(trifluoromethyl)-7,8-dihydro-6H-pyrazolo[1,5-a]pyrrolo[2,3-e]pyrimidine-6-carboxamido)nicotinic acid (30 mg, 61% yield) as a white solid. LC-MS: m/z 660 [M+H]⁺.

### Step 7: 4-chloro-6-((R)-2-chloro-8-methyl-8-(trifluoromethyl)-7,8-dihydro-6H-pyrazolo[1,5-a]pyrrolo[2,3-e]pyrimidine-6-carboxamido)-2-(((S)-pyrrolidin-3-yl)oxy)nicotinic acid

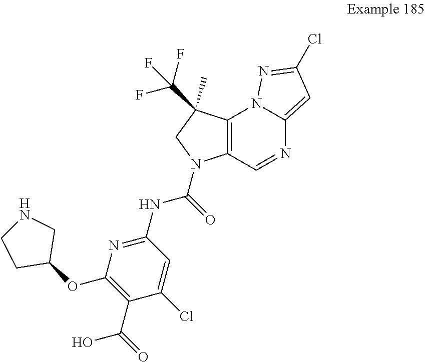 [SMILES: C[C@@]1(C(F)(F)F)CN(C(=O)Nc2cc(Cl)c(C(=O)O)c(O[C@H]3CCNC3)n2)c2cnc3cc(Cl)nn3c21]

To a stirred mixture of 2-(((S)-1-(tert-butoxycarbonyl)pyrrolidin-3-yl)oxy)-4-chloro-6-((R)-2-chloro-8-methyl-8-(trifluoromethyl)-7,8-dihydro-6H-pyrazolo[1,5-a]pyrrolo[2,3-e]pyrimidine-6-carboxamido)nicotinic acid (30 mg, 45 μmol) in dichloromethane (2 mL) was added TFA (0.4 mL). The mixture was stirred at 25° C. for 1 h. The reaction mixture was concentrated under vacuum. The residue was submitted to Prep-HPLC purification and the collected fractions were lyophilized to give 4-chloro-6-((R)-2-chloro-8-methyl-8-(trifluoromethyl)-7,8-dihydro-6H-pyrazolo[1,5-a]pyrrolo[2,3-e]pyrimidine-6-carboxamido)-2-(((S)-pyrrolidin-3-yl)oxy)nicotinic acid (4.4 mg, 17% yield) as a white solid. The enantiomer of Example 185 can be prepared analogously using Method M1 isomer 1.

Example 185: ¹H NMR (400 MHz, DMSO-d₆) δ:14.88 (br, 1H), 9.38 (s, 1H), 7.95 (br, 2H), 7.01 (s, 1H), 6.46 (s, 1H), 5.41-5.43 (m, 1H), 4.53 (d, J=11.6 Hz, 1H), 4.23 (d, J=11.6 Hz, 1H), 3.55-3.65 (m, 1H), 3.28 (s, 3H), 2.28-2.32 (m, 1H), 2.15-2.16 (m, 1H), 1.99 (s, 3H). LC-MS: m/z 560 [M+H]⁺.

### Method G6

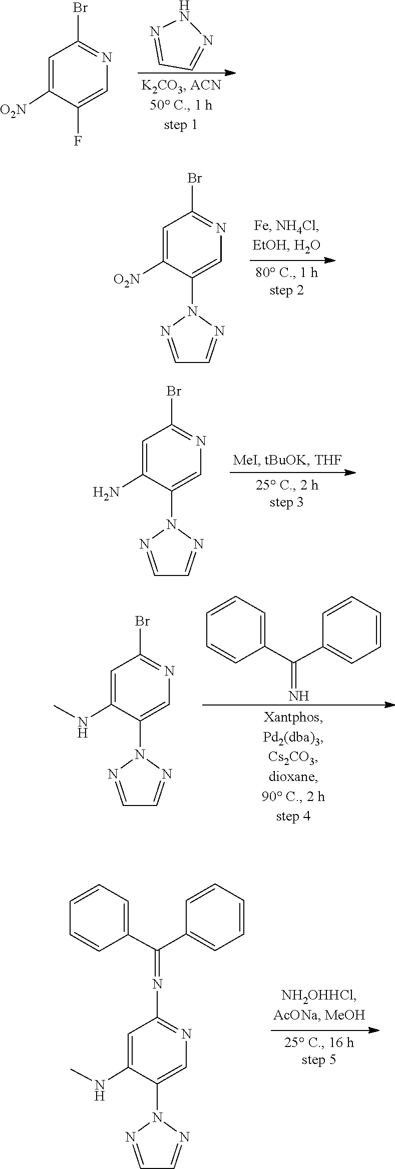

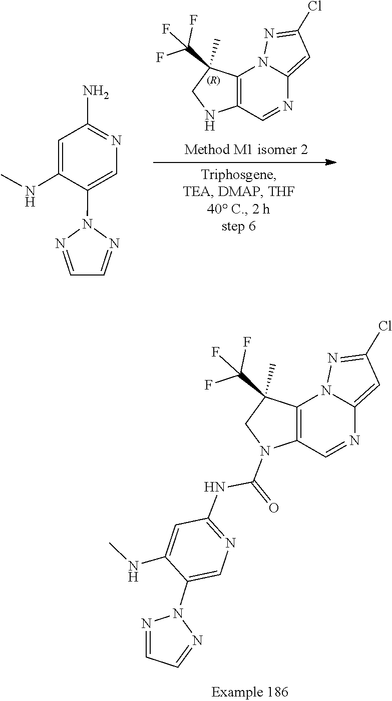

### Example 186: (R)-2-chloro-8-methyl-N-(4-(methylamino)-5-(2H-1,2,3-triazol-2-yl)pyridin-2-yl)-8-(trifluoromethyl)-7,8-dihydro-6H-pyrazolo[1,5-a]pyrrolo[2,3-e]pyrimidine-6-carboxamide

### Step 1: 2-bromo-4-nitro-5-(2H-1,2,3-triazol-2-yl)pyridine

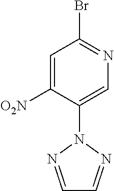 [SMILES: O=[N+]([O-])c1cc(Br)ncc1-n1nccn1]

To a stirred solution of 2-bromo-5-fluoro-4-nitropyridine (2.80 g, 12.6 mmol) in acetonitrile (20 mL) were added 2H-1,2,3-triazole (875 mg, 12.6 mmol) and K₂CO₃ (3.50 g, 25.2 mmol). The reaction mixture was stirred at 50° C. for 1 h. After cooled to 25° C., the reaction mixture was quenched with water (100 mL). The resulting mixture was extracted with ethyl acetate (3×100 mL). The combined organic layers were washed with brine (100 mL), dried over anhydrous sodium sulfate and concentrated under vacuum. The residue was purified by column chromatography using 60% petroleum ether and 40% ethyl acetate as eluent to afford 2-bromo-4-nitro-5-(2H-1,2,3-triazol-2-yl)pyridine (1.20 g, 35.% yield) as a white solid. ¹H NMR (400 MHz, Chloroform-d) δ: 9.14 (s, 1H), 7.93 (s, 2H), 7.81 (s, 1H). LC-MS: m/z 270 [M+H]⁺.

### Step 2: 2-bromo-5-(2H-1,2,3-triazol-2-yl)pyridin-4-amine

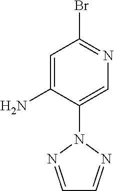 [SMILES: Nc1cc(Br)ncc1-n1nccn1]

To a stirred solution of 2-bromo-4-nitro-5-(2H-1,2,3-triazol-2-yl)pyridine (1.20 g, 4.4 mmol) in ethanol (60 mL) and water (20 mL) were added Fe (740 mg, 13.2 mmol) and NH₄Cl (1.17 g, 22.0 mmol) at 25° C. The reaction mixture was stirred at 80° C. for 1 h. After cooled to 25° C., the solid was filtered out. The filtrate was concentrated under vacuum to remove ethanol. The resulting solution was extracted with ethyl acetate (3×50 mL). The combined organic layers were washed with brine (100 mL), dried over anhydrous sodium sulfate and concentrated under vacuum. The residue was purified by column chromatography using 60% petroleum ether and 40% ethyl acetate as eluent to afford 2-bromo-5-(2H-1,2,3-triazol-2-yl)pyridin-4-amine (880 mg, 82% yield) as a white solid. ¹H NMR (400 MHz, Methanol-d₄) δ: 8.56 (s, 1H), 7.99 (s, 2H), 7.05 (s, 1H). LC-MS: m/z 240 [M+H]⁺.

### Step 3: 2-bromo-N-methyl-5-(2H-1,2,3-triazol-2-yl)pyridin-4-amine

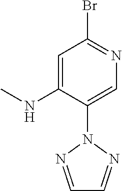

To a stirred solution of 2-bromo-5-(2H-1,2,3-triazol-2-yl)pyridin-4-amine (880 mg, 3.6 mmol) in tetrahydrofuran (30 mL) were added iodomethane (484 mg, 3.4 mmol) and potassium tert-butoxide (822 mg, 7.2 mmol). The mixture was stirred at 25° C. for 2 h. The resulting mixture was concentrated under vacuum. The residue was purified by Prep-HPLC and the collected fractions were concentrated to give 2-bromo-N-methyl-5-(2H-1,2,3-triazol-2-yl)pyridin-4-amine (520 mg, 55% yield) as a white solid. ¹H NMR (400 MHz, Chloroform-d) δ: 8.76 (s, 1H), 7.85 (s, 2H), 7.67 (s, 1H), 6.83 (br, 1H), 2.97 (d, J=5.2 Hz, 3H), 0.07 (s, 1H). LC-MS: m/z 254 [M+H]⁺.

### Step 4: 2-((diphenylmethylene)amino)-N-methyl-5-(2H-1,2,3-triazol-2-yl)pyridin-4-amine

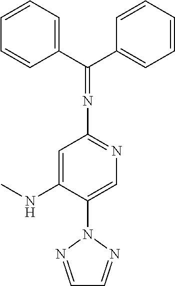 [SMILES: CNc1cc(N=C(c2ccccc2)c2ccccc2)ncc1-n1nccn1]

To a stirred solution of 2-bromo-N-methyl-5-(2H-1,2,3-triazol-2-yl)pyridin-4-amine (520 mg, 2.0 mmol) and diphenylmethanimine (370 mg, 2.0 mmol) in dioxane (40 mL) were added XantPhos (355 mg, 613.9 μmol), Pd₂(dba)₃ (235 mg, 409.3 μmol) and Cs₂CO₃ (2.00 g, 6.1 mmol). The reaction mixture was stirred at 90° C. for 2 h under nitrogen atmosphere. After cooled to 25° C., the solid was filtered out. The filtrate was concentrated under vacuum. The residue was purified by column chromatography using 90% petroleum ether and 10% ethyl acetate as eluent to afford 2-((diphenylmethylene)amino)-N-methyl-5-(2H-1,2,3-triazol-2-yl)pyridin-4-amine (160 mg, 22% yield) as a yellow solid. LC-MS: m/z 355 [M+H]⁺.

### Step 5: N⁴-methyl-5-(2H-1,2,3-triazol-2-yl)pyridine-2,4-diamine

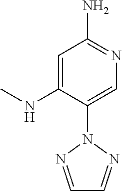

To a stirred solution of 2-((diphenylmethylene)amino)-N-methyl-5-(2H-1,2,3-triazol-2-yl)pyridin-4-amine (160 mg, 451.4 μmol) in methanol (10 mL) were added hydroxylamine hydrochloride (62 mg, 892.2 μmol) and sodium acetate (92 mg, 1.1 mmol). The reaction mixture was stirred at 25° C. for 16 h. The resulting mixture was concentrated under vacuum. The residue was purified by column chromatography using 60% petroleum ether and 40% ethyl acetate as eluent to afford N⁴-methyl-5-(2H-1,2,3-triazol-2-yl)pyridine-2,4-diamine (80 mg, 93% yield) as a yellow solid. ¹H NMR (400 MHz, Methanol-d₄) δ: 8.41 (s, 1H), 7.93 (s, 2H), 5.99 (s, 1H), 2.74 (s, 3H). LC-MS: m/z 191 [M+H]⁺.

### Step 6: (R)-2-chloro-8-methyl-N-(4-(methylamino)-5-(2H-1,2,3-triazol-2-yl)pyridin-2-yl)-8-(trifluoromethyl)-7,8-dihydro-6H-pyrazolo[1,5-a]pyrrolo[2,3-e]pyrimidine-6-carboxamide

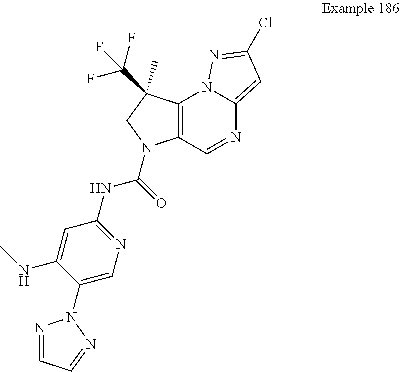 [SMILES: CNc1cc(NC(=O)N2C[C@@](C)(C(F)(F)F)c3c2cnc2cc(Cl)nn32)ncc1-n1nccn1]

To a stirred solution of Method M1 isomer 2 (40 mg, 144.5 μmol) in tetrahydrofuran (5 mL) were added triphosgene (25 mg, 86.7 μmol) and TEA (22 mg, 216 μmol). The resulting mixture was stirred at 25° C. for 0.5 h and then filtered. The resulting filtrate was added to a solution of N⁴-methyl-5-(2H-1,2,3-triazol-2-yl)pyridine-2,4-diamine (27 mg, 144.5 μmol) in tetrahydrofuran (2 mL). To this solution was then added TEA (146 mg, 1.4 mmol) and N,N-dimethylpyridin-4-amine (35 mg, 289 μmol). The mixture was stirred at 40° C. for 2 h. The reaction mixture was quenched with water (10 mL), and the resulting solution was extracted with ethyl acetate (3×10 mL). The combined organic layers were washed with brine (20 mL), dried over anhydrous sodium sulfate and concentrated under vacuum. The residue was submitted to Prep-HPLC purification and the collected fractions were lyophilized to give (R)-2-chloro-8-methyl-N-(4-(methylamino)-5-(2H-1,2,3-triazol-2-yl)pyridin-2-yl)-8-(trifluoromethyl)-7,8-dihydro-6H-pyrazolo[1,5-a]pyrrolo[2,3-e]pyrimidine-6-carboxamide (10.7 mg, 14% yield) as a white solid. The enantiomer of Example 186 can be prepared analogously using Method M1 isomer 1.

Example 186: ¹H NMR (400 MHz, DMSO-d₆) δ: 9.85 (br, 1H), 9.35 (s, 1H), 8.33 (s, 1H), 8.15 (s, 2H), 7.37 (s, 1H), 7.00-7.05 (m, 2H), 4.98 (d, J=11.6 Hz, 1H), 4.24 (d, J=11.6 Hz, 1H), 2.86 (d, J=4.8 Hz, 3H), 1.94 (s, 3H). LC-MS: m/z 493 [M+H]⁺.

### Method H6

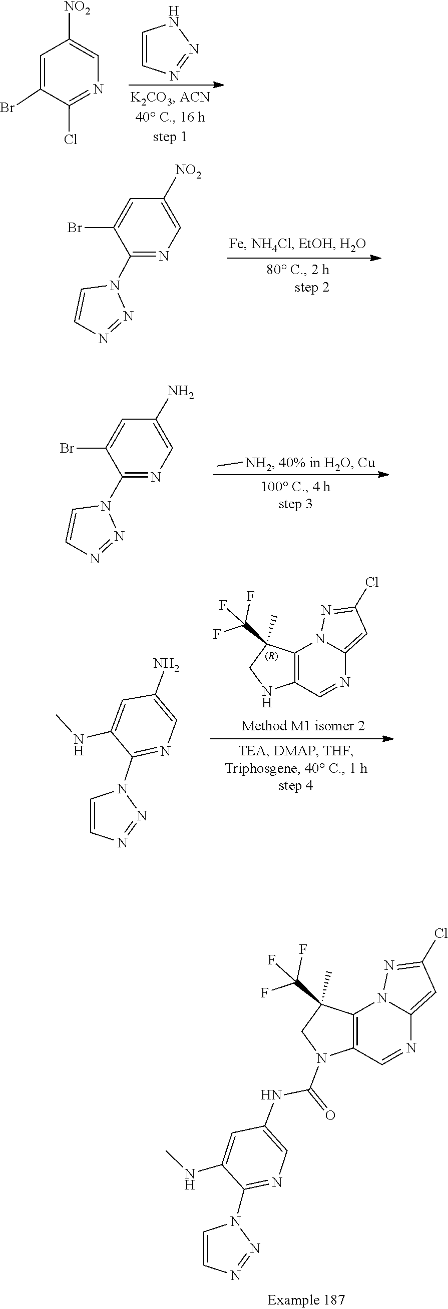

### Examples 187: (R)-2-chloro-8-methyl-N-(5-(methylamino)-6-(1H-1,2,3-triazol-1-yl)pyridin-3-yl)-8-(trifluoromethyl)-7,8-dihydro-6H-pyrazolo[1,5-a]pyrrolo[2,3-e]pyrimidine-6-carboxamide

### Step 1: 3-bromo-5-nitro-2-(1H-1,2,3-triazol-1-yl)pyridine

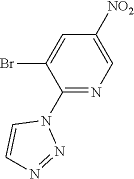 [SMILES: O=[N+]([O-])c1cnc(-n2ccnn2)c(Br)c1]

To a stirred solution of 3-bromo-2-chloro-5-nitropyridine (10.0 g, 42.4 mmol) in acetonitrile (200 mL) was added 2H-1,2,3-triazole (3.2 g, 46.6 mmol) and K₂CO₃ (11.7 g, 84.7 mmol). The resulting mixture was stirred at 40° C. for 16 h. The mixture was cooled to 25° C. The reaction mixture was filtered and the collected solid was washed with ethyl acetate (3×200 mL). The combined organic layers were dried over anhydrous sodium sulfate and concentrated under vacuum. The residue was purified by column chromatography on silica gel using 75% petroleum ether and 25% ethyl acetate as eluent to afford 3-bromo-5-nitro-2-(1H-1,2,3-triazol-1-yl)pyridine (2.0 g, 17% yield) as a yellow solid. LC-MS: m/z 270 [M+H]⁺.

### Step 2: 5-bromo-6-(1H-1,2,3-triazol-1-yl)pyridin-3-amine

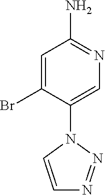 [SMILES: Nc1cc(Br)c(-n2ccnn2)cn1]

To a solution of 3-bromo-5-nitro-2-(1H-1,2,3-triazol-1-yl)pyridine (1.0 g, 3.7 mmol) in ethanol (45 mL) and water (15 mL) were added Fe (1.0 g, 18.6 mmol), NH₄Cl (0.8 g, 14.8 mmol). The resulting mixture was stirred at 80° C. for 2 h. After cooled to 25° C., the solid was filtered out. The filtrate was concentrated under vacuum to remove ethanol. The resulting solution was diluted with water (50 mL), extracted with ethyl acetate (3×50 mL). The combined organic layers were washed with brine (100 mL), dried over anhydrous sodium sulfate and concentrated under vacuum. The residue was purified by column chromatography on silica gel using 50% petroleum ether and 50% ethyl acetate as eluent to afford 5-bromo-6-(1H-1,2,3-triazol-1-yl)pyridin-3-amine (0.8 g, 89% yield) as a yellow oil. LC-MS: m/z 240 [M+H]⁺.

### Step 3: N³-methyl-2-(1H-1,2,3-triazol-1-yl)pyridine-3,5-diamine

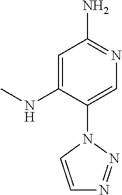

To a stirred solution of 5-(difluoromethyl)-1H-pyrazol-3-amine (500 mg, 3.3 mmol) in methylamine (4 mL, 40% in water) was added copper (8 mg, 0.1 mmol). The reaction mixture was stirred at 100° C. for 4 h. The mixture was cooled to 25° C. The reaction mixture was quenched with water (20 mL). The resulting solution was extracted with ethyl acetate (3×50 mL). The combined organic layers were dried over anhydrous sodium sulfate and concentrated under vacuum. The residue was purified by column chromatography on silica gel using 90% dichloromethane and 10% methanol as eluent to afford N³-methyl-2-(1H-1,2,3-triazol-1-yl)pyridine-3,5-diamine (280 mg, 71% yield) as a yellow solid. LC-MS: m/z 191 [M+H]⁺.

### Step 4: (R)-2-chloro-8-methyl-N-(5-(methylamino)-6-(1H-1,2,3-triazol-1-yl)pyridin-3-yl)-8-(trifluoromethyl)-7,8-dihydro-6H-pyrazolo[1,5-a]pyrrolo[2,3-e]pyrimidine-6-carboxamide

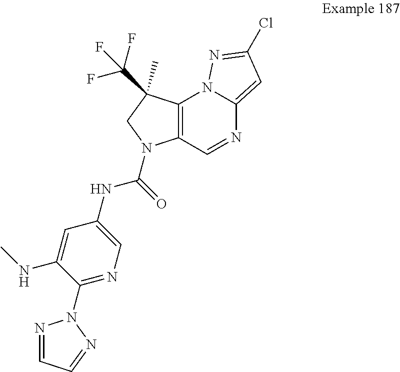 [SMILES: CNc1cc(NC(=O)N2C[C@@](C)(C(F)(F)F)c3c2cnc2cc(Cl)nn32)cnc1-n1nccn1]

To a stirred solution of N³-methyl-2-(1H-1,2,3-triazol-1-yl)pyridine-3,5-diamine (42 mg, 217 μmol) in tetrahydrofran (8 mL) were added triphosgene (32 mg, 108 μmol) and TEA (22 mg, 217.4 μmol) at 25° C. The resulting mixture was stirred at 25° C. for 0.5 h and then filtered. The resulting filtrate was added to a solution of Method M1 isomer 2 (48 mg, 173 μmol) in tetrahydrofuran (1 mL). To this solution was then added TEA (220 mg, 2.2 mmol) and N,N-dimethylpyridin-4-amine (53 mg, 434 μmol). The reaction mixture was stirred at 40° C. for 1 h. The reaction mixture was quenched with water (20 mL). The resulting solution was extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate as eluent to afford crude product. The crude product was submitted to Prep-HPLC purification and the collected fractions were lyophilized to give (R)-2-chloro-8-methyl-N-(5-(methylamino)-6-(1H-1,2,3-triazol-1-yl)pyridin-3-yl)-8-(trifluoromethyl)-7,8-dihydro-6H-pyrazolo[1,5-a]pyrrolo[2,3-e]pyrimidine-6-carboxamide (23 mg, 31% yield) as a white solid. The enantiomer of Example 187 can be prepared analogously using Method M1 isomer 1.

Example 187: ¹H NMR (400 MHz, DMSO-d₆) δ: 9.37 (s, 1H), 9.26 (br, 1H), 8.70 (d, J=1.2 Hz, 1H), 8.10 (d, J=2.0 Hz, 1H), 7.98 (d, J=1.2 Hz, 1H), 7.57 (d, J=2.0 Hz, 1H), 7.06 (s, 1H), 6.76 (br, 1H), 4.86 (d, J=11.2 Hz, 1H), 4.28 (d, J=11.2 Hz, 1H), 2.88 (d, J=4.0 Hz, 3H), 1.99 (s, 3H). LC-MS: m/z 493 [M+H]⁺.

### Method I6

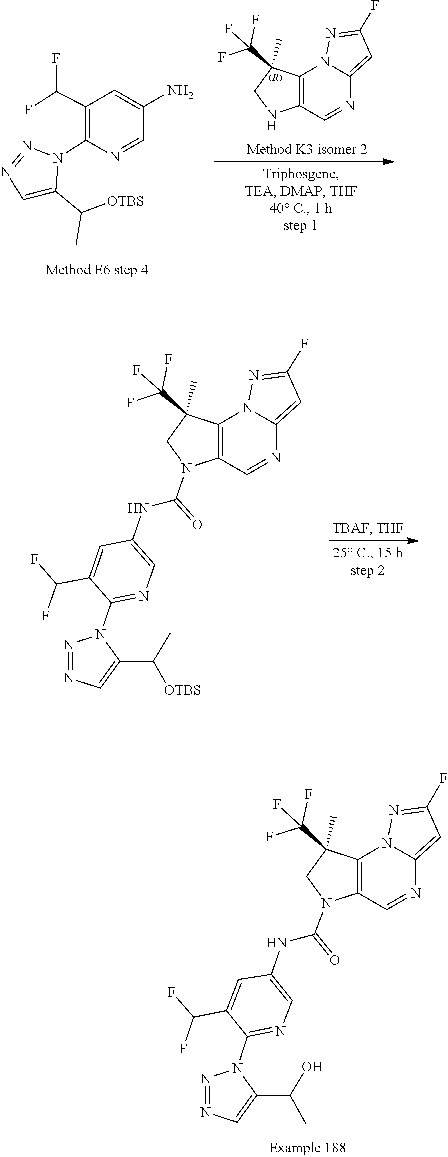

### Example 188: (8R)—N-(5-(difluoromethyl)-6-(5-(1-hydroxyethyl)-1H-1,2,3-triazol-1-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

### Step 1: (8R)—N-(6-(5-(1-((tert-butyldimethylsilyl)oxy)ethyl)-1H-1,2,3-triazol-1-yl)-5-(difluoromethyl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

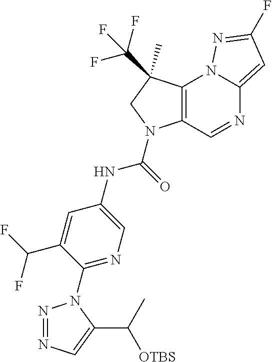 [SMILES: CC(O[Si](C)(C)C(C)(C)C)c1cnnn1-c1ncc(NC(=O)N2C[C@@](C)(C(F)(F)F)c3c2cnc2cc(F)nn32)cc1C(F)F]

To a stirred solution of 6-(5-(1-((tert-butyldimethylsilyl)oxy)ethyl)-1H-1,2,3-triazol-1-yl)-5-(difluoromethyl)pyridin-3-amine (prepared in analogy to Method M4 step 8; 30 mg, 81.2 μmol) in tetrahydrofuran (3 mL) were added TEA (12 mg, 121.8 μmol) and triphosgene (14 mg, 48.7 μmol). The resulting mixture was stirred at 25° C. for 0.5 h and then filtered. The filtrate was added to a solution of (R)-2-fluoro-8-methyl-8-(trifluoromethyl)-7,8-dihydro-6H-pyrazolo[1,5-a]pyrrolo[2,3-e]pyrimidine (Method K3 isomer 2; 15 mg, 57.7 μmol) in tetrahydrofuran (1 mL). To this solution were added N,N-dimethylpyridin-4-amine (20 mg, 162.4 μmol) and TEA (82 mg, 800 μmol). The mixture was stirred at 40° C. for 1 h. The reaction mixture was concentrated under vacuum. The residue was purified by Prep-TLC using 50% petroleum ether and 50% ethyl acetate as eluent to afford (8R)—N-(6-(5-(1-((tert-butyldimethylsilyl)oxy)ethyl)-1H-1,2,3-triazol-1-yl)-5-(difluoromethyl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide (25 mg, 45% yield) as a white solid. LC-MS: m/z 656 [M+H]⁺.

### Step 2: (8R)—N-(5-(difluoromethyl)-6-(5-(1-hydroxyethyl)-1H-1,2,3-triazol-1-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

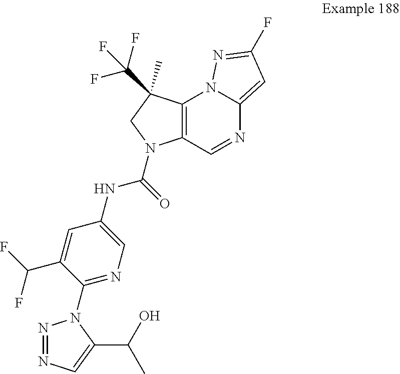 [SMILES: CC(O)c1cnnn1-c1ncc(NC(=O)N2C[C@@](C)(C(F)(F)F)c3c2cnc2cc(F)nn32)cc1C(F)F]

To a stirred solution of (8R)—N-(6-(5-(1-((tert-butyldimethylsilyl)oxy)ethyl)-1H-1,2,3-triazol-1-yl)-5-(difluoromethyl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide (25 mg, 38.1 μmol) in tetrahydrofuran (5 mL) was added tetrabutylammonium fluoride (1 mL, 1 M in tetrahydrofuran) at 25° C. The reaction was stirred at 25° C. for 15 h. The solvent was removed under vacuum. The mixture was concentrated under vacuum. The residue was submitted to Prep-HPLC purification and the collected fractions were lyophilized to afford (8R)—N-(5-(difluoromethyl)-6-(5-(1-hydroxyethyl)-1H-1,2,3-triazol-1-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide (16.8 mg, 81% yield) as a white solid. The corresponding stereoisomers of Example 188 with respect to the chiral center attached to the trifluoromethyl group can be prepared analogously using Method K3 isomer 1.

Example 188: ¹H NMR (400 MHz, DMSO-d₆) δ: 9.67 (s, 1H), 9.36 (s, 1H), 8.98 (d, J=2.4 Hz, 1H), 8.60 (d, J=2.4 Hz, 1H), 8.53 (s, 1H), 7.45 (t, J=54.0 Hz, 1H), 6.70 (d, J=4.8 Hz, 1H), 5.43 (d, J=5.2 Hz, 1H), 4.92-4.98 (m, 1H), 4.85 (d, J=11.6 Hz, 1H), 4.31 (d, J=11.6 Hz, 1H), 1.98 (s, 3H), 1.49 (d, J=6.4 Hz, 3H). LC-MS: m/z 542 [M+H]⁺.

### Method J6

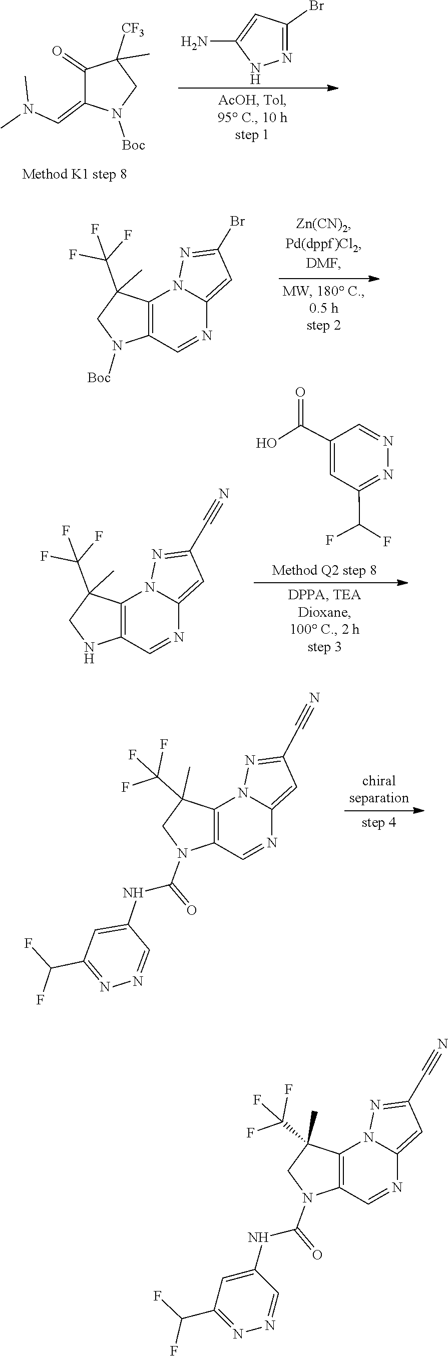

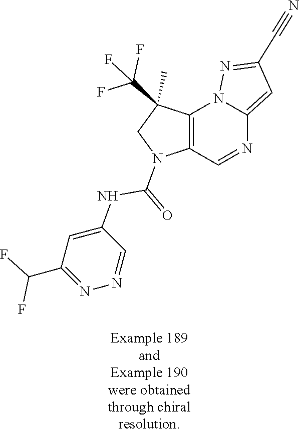 [SMILES: C[C@@]1(C(F)(F)F)CN(C(=O)Nc2cnnc(C(F)F)c2)c2cnc3cc(C#N)nn3c21]

### Examples 189 and 190: Single enantiomers obtained from a racemic mixture containing (S)-2-cyano-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide and (R)-2-cyano-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide

### Step 1: tert-butyl 2-bromo-8-methyl-8-(trifluoromethyl)-7,8-dihydro-6H-pyrazolo[1,5-a]pyrrolo[2,3-e]pyrimidine-6-carboxylate

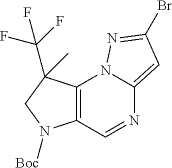 [SMILES: CC(C)(C)OC(=O)N1CC(C)(C(F)(F)F)c2c1cnc1cc(Br)nn21]

To a stirred solution of 3-bromo-1H-pyrazol-5-amine (280 mg, 1.7 mmol) in toluene (10 mL) was added acetic acid (1 mL) and tert-butyl (E)-2-((dimethylamino)methylene)-4-methyl-3-oxo-4-(trifluoromethyl)pyrrolidine-1-carboxylate (Method K1 step 8; 557 mg, 1.7 mmol). The reaction mixture was stirred at 95° C. for 10 h. The mixture was concentrated under vacuum. The residue was diluted with saturated aqueous NaHCO₃ solution (10 mL). The resulting solution was extracted with ethyl acetate (3×10 mL). The combined organic layers were dried over anhydrous sodium sulfate and concentrated under vacuum. The residue was purified by column chromatography using 80% petroleum ether and 20% ethyl acetate as eluent to afford tert-butyl 2-bromo-8-methyl-8-(trifluoromethyl)-7,8-dihydro-6H-pyrazolo[1,5-a]pyrrolo[2,3-e]pyrimidine-6-carboxylate (420 mg, 57% yield) as a yellow solid. LC-MS: m/z 421 [M+H]⁺.

### Step 2: 8-methyl-8-(trifluoromethyl)-7,8-dihydro-6H-pyrazolo[1,5-a]pyrrolo[2,3-e]pyrimidine-2-carbonitrile

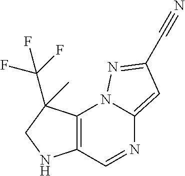 [SMILES: CC1(C(F)(F)F)CNc2cnc3cc(C#N)nn3c21]

To a stirred solution of tert-butyl 2-bromo-8-methyl-8-(trifluoromethyl)-7,8-dihydro-6H-pyrazolo[1,5-a]pyrrolo[2,3-e]pyrimidine-6-carboxylate (200 mg, 474.8 μmol) in N,N-Dimethylformamide (5 mL) were added Zn(CN)₂ (112 mg, 949.6 μmol) and PdCl₂(dppf) (52 mg, 71.2 μmol) under nitrogen atmosphere. The final reaction mixture was irradiated with microwave radiation at 180° C. for 0.5 h. After cooled to 25° C., the solid was filtered out. The filtrate was concentrated under vacuum. The residue was purified by column chromatography on silica gel using 90% dichloromethane and 10% methanol as eluent to afford 8-methyl-8-(trifluoromethyl)-7,8-dihydro-6H-pyrazolo[1,5-a]pyrrolo[2,3-e]pyrimidine-2-carbonitrile (100 mg, 76% yield) as a yellow solid. LC-MS: m/z 268 [M+H]⁺.

### Step 3: 2-cyano-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide

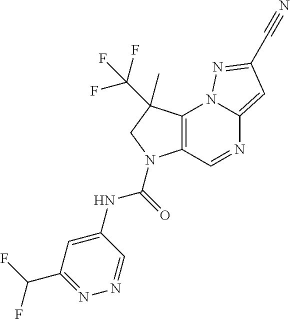 [SMILES: CC1(C(F)(F)F)CN(C(=O)Nc2cnnc(C(F)F)c2)c2cnc3cc(C#N)nn3c21]

To a stirred solution of 8-methyl-8-(trifluoromethyl)-7,8-dihydro-6H-pyrazolo[1,5-a]pyrrolo[2,3-e]pyrimidine-2-carbonitrile (50 mg, 187.1 μmol) and 6-(difluoromethyl)pyridazine-4-carboxylic acid (Method Q2 step 8; 33 mg, 187.1 μmol) in dioxane (5 mL) was added DPPA (55 mg, 224.5 μmol) and TEA (95 mg, 935.6 μmol) at 25° C. The resulting mixture was stirred at 100° C. for 2 h. The reaction was cooled to 25° C. The resulting mixture was concentrated under vacuum. The residue was diluted with water (10 mL), and the resulting solution was extracted with ethyl acetate (3×10 mL). The combined organic layers were dried over anhydrous sodium sulfate and concentrated under vacuum. The residue was purified by Prep-HPLC and the collected fractions were lyophilized to afford 2-cyano-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide (15 mg, 18% yield) as a white solid. ¹H NMR (400 MHz, DMSO-d₆) δ: 9.99 (s, 1H), 9.51 (s, 2H), 8.21 (d, J=2.8 Hz, 1H), 7.78 (s, 1H), 7.25 (t, J=54.4 Hz, 1H), 4.91 (d, J=11.6 Hz, 1H), 4.35 (d, J=11.6 Hz, 1H), 1.99 (s, 3H). LC-MS: m/z 439 [M+H]⁺.

### Step 4: Separation of enantiomers to obtain (S)-2-cyano-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide and (R)-2-cyano-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide

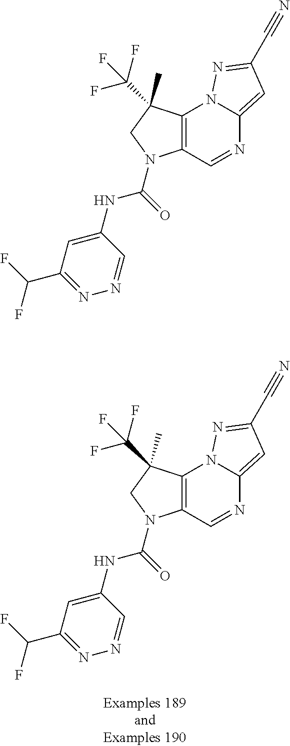 [SMILES: C[C@]1(C(F)(F)F)CN(C(=O)Nc2cnnc(C(F)F)c2)c2cnc3cc(C#N)nn3c21 ; C[C@@]1(C(F)(F)F)CN(C(=O)Nc2cnnc(C(F)F)c2)c2cnc3cc(C#N)nn3c21]

2-cyano-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide (15 mg, 34.2 μmol) was submitted to chiral-HPLC: Column: Lux Sum Cellulose-2, 2.12*25 cm, 5 μm; Mobile Phase A:Hex(0.5% 2M NH3-MeOH)-HPLC, Mobile Phase B:EtOH—HPLC; Flow rate: 20 mL/min; Gradient: 40 B to 40 B in 25 min; 254/220 nm; RT1:13.242; RT2:19.844; Injection Volume: 1 ml; Number Of Runs: 2. The first eluting isomer was concentrated and lyophilized to afford Example 189 (6.7 mg, 44% yield) as a light yellow solid. The second eluting isomer was concentrated and lyophilized to afford Example 190 (6.4 mg, 42% yield) as a light-yellow solid. Examples 189 and 190 are enantiomers, but their absolute stereochemistry is not yet known.

Example 189: ¹H NMR (300 MHz, Methanol-d₄) δ: 9.57 (s, 1H), 9.48 (d, J=2.4 Hz, 1H), 8.34 (d, J=2.4 Hz, 1H), 7.40 (s, 1H), 6.96 (t, J=54.3 Hz, 1H), 4.85 (d, J=10.5 Hz, 1H), 4.28 (d, J=10.5 Hz, 1H), 2.06 (s, 3H). LC-MS: m/z 439 [M+H]⁺.

Example 190: ¹H NMR (300 MHz, Methanol-d₄) δ: 9.57 (s, 1H), 9.49 (d, J=2.4 Hz, 1H), 8.34 (d, J=2.4 Hz, 1H), 7.40 (s, 1H), 6.97 (t, J=54.3 Hz, 1H), 4.85 (d, J=11.7 Hz, 1H), 4.29 (d, J=11.7 Hz, 1H), 2.06 (s, 3H). LC-MS: m/z 439 [M+H]⁺.

### Method K6

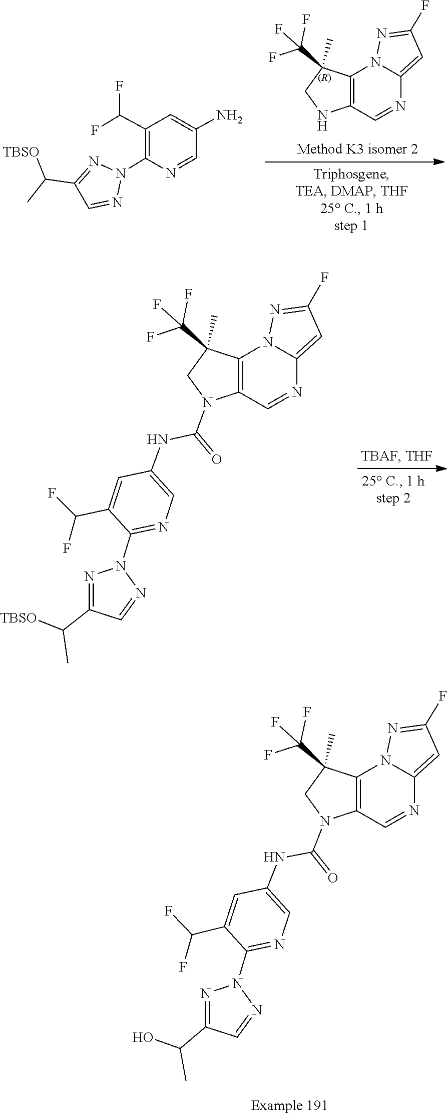

### Example 191: (8R)—N-(5-(difluoromethyl)-6-(4-(1-hydroxyethyl)-2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

### Step 1: (8R)—N-(6-(4-(1-((tert-butyldimethylsilyl)oxy)ethyl)-2H-1,2,3-triazol-2-yl)-5-(difluoromethyl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

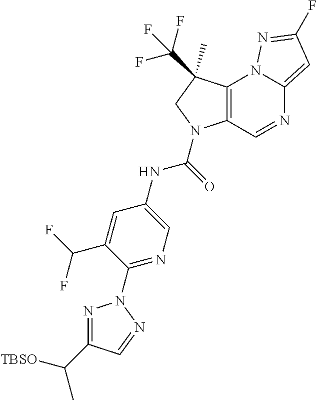 [SMILES: CC(O[Si](C)(C)C(C)(C)C)c1cnn(-c2ncc(NC(=O)N3C[C@@](C)(C(F)(F)F)c4c3cnc3cc(F)nn43)cc2C(F)F)n1]

To a stirred mixture of 6-(4-(1-((tert-butyldimethylsilyl)oxy)ethyl)-2H-1,2,3-triazol-2-yl)-5-(difluoromethyl)pyridin-3-amine (40 mg, 108.2 μmol) in tetrahydrofuran (5 mL) were added triphosgene (19 mg, 64.96 μmol) and TEA (16 mg, 162.4 μmol) at 25° C. The resulting mixture was stirred at 25° C. for 0.5 h and then filtered. The resulting filtrate was added to a solution of (R)-2-fluoro-8-methyl-8-(trifluoromethyl)-7,8-dihydro-6H-pyrazolo[1,5-a]pyrrolo[2,3-e]pyrimidine (20 mg, 75.8 μmol) in tetrahydrofuran (1 mL). To this solution was then added TEA (109 mg, 1.1 mmol) and N,N-dimethylpyridin-4-amine (26 mg, 216.5 μmol). The mixture was stirred at 25° C. for 1 h. The reaction mixture was concentrated under vacuum. The residue was purified by Prep-TLC using 50% petroleum ether and 50% ethyl acetate as eluent to afford (8R)—N-(6-(4-(1-((tert-butyldimethylsilyl)oxy)ethyl)-2H-1,2,3-triazol-2-yl)-5-(difluoromethyl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide (15 mg, 21% yield) as a white solid. LC-MS: m/z 656 [M+H]⁺.

### Step 2: (8R)—N-(5-(difluoromethyl)-6-(4-(1-hydroxyethyl)-2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

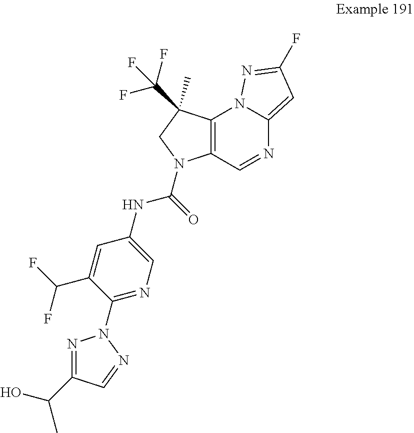 [SMILES: CC(O)c1cnn(-c2ncc(NC(=O)N3C[C@@](C)(C(F)(F)F)c4c3cnc3cc(F)nn43)cc2C(F)F)n1]

To a stirred mixture of (8R)—N-(6-(4-(1-((tert-butyldimethylsilyl)oxy)ethyl)-2H-1,2,3-triazol-2-yl)-5-(difluoromethyl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide (15 mg, 22.88 umol) in tetrahydrofuran (1 mL) was added tetrabutylammonium fluoride (1 mL, 1 M in tetrahydrofuran). The reaction mixture was stirred at 25° C. for 1 h. The reaction mixture was concentrated. The residue was purified by Prep-TLC using 100% ethyl acetate as eluent to afford crude product. The crude product was submitted to Prep-HPLC purification and the collected fractions were lyophilized to afford (8R)—N-(5-(difluoromethyl)-6-(4-(1-hydroxyethyl)-2H-1,2,3-triazol-2-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide (4.5 mg, 36% yield) as an off-white solid. The corresponding stereoisomers of Example 191 with respect to the chiral center attached to the trifluoromethyl group can be prepared analogously using Method K3 isomer 1.

Example 191: ¹H NMR (400 MHz, DMSO-d₆) δ: 9.65 (s, 1H), 9.36 (s, 1H), 8.95 (d, J=2.4 Hz, 1H), 8.57 (d, J=2.4 Hz, 1H), 8.09 (s, 1H), 7.41 (t, J=54.4 Hz, 1H), 6.69 (d, J=4.8 Hz, 1H), 5.55 (d, J=5.2 Hz, 1H), 4.93-4.99 (m, 1H), 4.86 (d, J=11.6 Hz, 1H), 4.31 (d, J=11.6 Hz, 1H), 1.97 (s, 3H), 1.48 (d, J=6.4 Hz, 3H). LC-MS: m/z 542 [M+H]⁺.

### Method L6

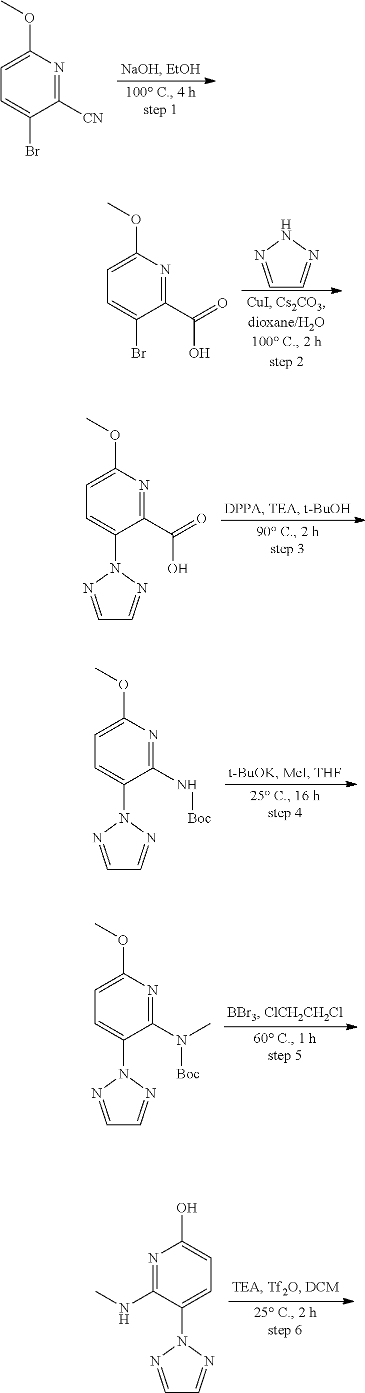

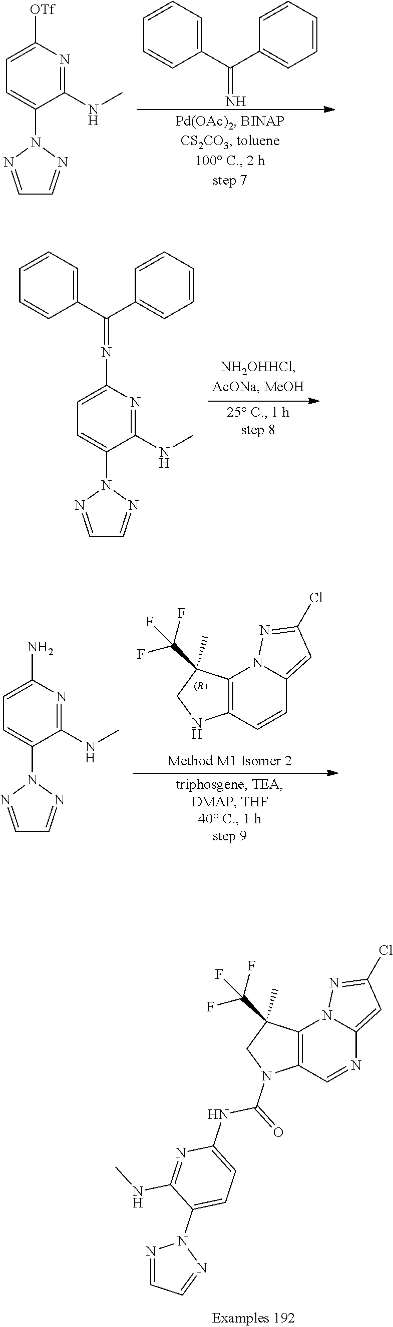

### Examples 192: (R)-2-chloro-8-methyl-N-(6-(methylamino)-5-(2H-1,2,3-triazol-2-yl)pyridin-2-yl)-8-(trifluoromethyl)-7,8-dihydro-6H-pyrazolo[1,5-a]pyrrolo[2,3-e]pyrimidine-6-carboxamide

### Step 1: 3-bromo-6-methoxypicolinic acid

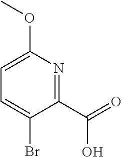

To a stirred solution of 3-bromo-6-methoxypicolinonitrile (850 mg, 3.9 mml) in ethanol (15 mL) was added sodium hydroxide (1.60 g, 39.9 mmol) at 25° C. The resulting mixture was stirred at 100° C. for 4 h. After cooled to 25° C., the resulting solution was quenched with water (50 mL). The pH was adjusted to 3 with HCl (1 M). The mixture was extracted with ethyl acetate (3×50 mL). The combined organic layers were washed with brine (60 mL), dried over anhydrous sodium sulfate and concentrated under vacuum to afford 3-bromo-6-methoxypicolinic acid (850 mg, 75% yield) as a yellow solid. LC-MS: m/z 232 [M+H]⁺.

### Step 2: 6-methoxy-3-(2H-1,2,3-triazol-2-yl)picolinic acid

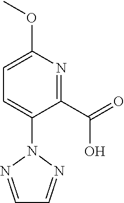 [SMILES: COc1ccc(-n2nccn2)c(C(=O)O)n1]

To a stirred mixture of 3-bromo-6-methoxypicolinic acid (500 mg, 2.1 mmol) and 2H-1,2,3-triazole (297 mg, 4.3 mmol) in dioxane (10 mL) and water (0.05 mL) were added (1S,2R)-cyclohexane-1,2-diamine (49 mg, 430.9 μmol), copper(I) iodide (82 mg, 430.9 μmol) and Cs₂CO₃ (1.4 g, 4.3 mmol) at 25° C. The resulting mixture was stirred at 100° C. for 2 h. After cooled to 25° C., the resulting solution was quenched with water (50 mL). The pH was adjusted to 5 with HCl (1 M). The resulting mixture was extracted with ethyl acetate (4×80 mL). The combined organic layers were washed with brine (100 mL), dried over anhydrous sodium sulfate and concentrated under vacuum. The residue was purified by Prep-HPLC purification and the collected fractions were lyophilized to give 6-methoxy-3-(2H-1,2,3-triazol-2-yl)picolinic acid (250 mg, 52% yield) as a yellow solid. ¹H NMR (400 MHz, DMSO-d₆) δ:13.43 (s, 1H), 8.17-8.20 (m, 1H), 8.11 (s, 2H), 7.14-7.16 (m, 1H), 3.93 (s, 3H). LC-MS: m/z 221 [M+H]⁺.

### Step 3: tert-butyl (6-methoxy-3-(2H-1,2,3-triazol-2-yl)pyridin-2-yl)carbamate

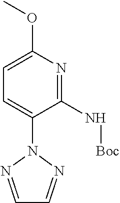 [SMILES: COc1ccc(-n2nccn2)c(NC(=O)OC(C)(C)C)n1]

To a stirred solution of 6-methoxy-3-(2H-1,2,3-triazol-2-yl)picolinic acid (500 mg, 2.2 mmol) in 2-methylpropan-2-ol (10 mL) were added TEA (758 mg, 7.4 mmol) and diphenylphosphoryl azide (2.1 g, 7.4 mmol) at 25° C. The resulting mixture was stirred at 90° C. for 2 h. After cooled to 25° C., the reaction mixture was quenched with water (100 mL). The resulting mixture was extracted with ethyl acetate (3×150 mL). The combined organic layers were washed with brine (100 mL), dried over anhydrous sodium sulfate and concentrated under vacuum. The residue was purified by column chromatography on silica gel using 80% petroleum ether and 20% ethyl acetate as eluent to afford tert-butyl (6-methoxy-3-(2H-1,2,3-triazol-2-yl)pyridin-2-yl)carbamate (450 mg, 68% yield) as a yellow solid. ¹H NMR (400 MHz, DMSO-d₆) δ: 9.59 (br, 1H), 8.03 (s, 2H), 8.01 (d, J=8.8 Hz, 1H), 6.74 (d, J=8.8 Hz, 1H), 3.88 (s, 3H), 1.28 (s, 9H). LC-MS: m/z 292 [M+H]⁺.

### Step 4: tert-butyl (6-methoxy-3-(2H-1,2,3-triazol-2-yl)pyridin-2-yl)(methyl)carbamate

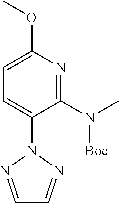 [SMILES: COc1ccc(-n2nccn2)c(N(C)C(=O)OC(C)(C)C)n1]

To a stirred solution of tert-butyl (6-methoxy-3-(2H-1,2,3-triazol-2-yl)pyridin-2-yl)(methyl)carbamate (2.3 g, 7.9 mmol) in tetrahydrofuran (120 mL) were added potassium tert-butoxide (1.7 g, 15.7 mmol) and iodomethane (3.3 g, 23.6 mmol) at 25° C. The resulting mixture was stirred at 25° C. for 16 h. The reaction mixture was quenched with water (300 mL). The resulting solution was extracted with ethyl acetate (3×300 mL). The combined organic layers were washed with brine (300 mL), dried over anhydrous sodium sulfate and concentrated under vacuum. The residue was purified by column chromatography on silica gel using 70% petroleum ether and 30% ethyl acetate as eluent to afford tert-butyl (6-methoxy-3-(2H-1,2,3-triazol-2-yl)pyridin-2-yl)(methyl)carbamate (2 g, 82% yield) as a yellow oil. ¹H NMR (300 MHz, DMSO-d₆) δ: 8.12 (s, 2H), 8.09 (d, J=8.7 Hz, 1H), 6.94 (d, J=8.7 Hz, 1H), 3.93 (s, 3H), 3.33 (s, 3H), 1.08 (s, 9H). LC-MS: m/z 306 [M+H]⁺.

### Step 5: 6-(methylamino)-5-(2H-1,2,3-triazol-2-yl)pyridin-2-ol

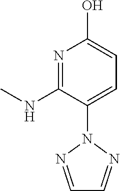

To a stirred solution of tert-butyl (6-methoxy-3-(2H-1,2,3-triazol-2-yl)pyridin-2-yl)(methyl)carbamate (1.2 g, 3.9 mmol) in 1,2-dichloroethane (15 mL) was added tribromoborane (11.7 mL, 11.7 mmol, 1 M in dichloromethane) dropwise at 0° C. under nitrogen atmosphere. The resulting mixture was stirred at 60° C. for 1 h. The reaction was quenched with methanol (10 mL) at 0° C. The resulting mixture was concentrated under vacuum. The residue was purified by Prep-HPLC purification and the collected fractions were concentrated under vacuum to afford 6-(methylamino)-5-(2H-1,2,3-triazol-2-yl)pyridin-2-ol (400 mg, 53% yield) as a yellow oil. H NMR (400 MHz, DMSO-d₆) δ: 8.03 (s, 2H), 7.75 (d, J=8.4 Hz, 1H), 5.86 (d, J=8.4 Hz, 1H), 2.88 (s, 3H). LC-MS: m/z 192 [M+H]⁺.

### Step 6: 6-(methylamino)-5-(2H-1,2,3-triazol-2-yl)pyridin-2-yl trifluoromethanesulfonate

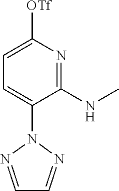 [SMILES: CNc1nc(OS(=O)(=O)C(F)(F)F)ccc1-n1nccn1]

To a stirred solution of 6-(methylamino)-5-(2H-1,2,3-triazol-2-yl)pyridin-2-ol (430 mg, 2.2 mmol) in dichloromethane (15 mL) was added TEA (682 mg, 6.7 mmol). Trifluoromethanesulfonic anhydride (951 mg, 3.3 mmol) was added at 0° C. under nitrogen atmosphere. The resulting mixture was stirred at 25° C. for 2 h. The reaction mixture was quenched with water (100 mL). The resulting solution was extracted with ethyl acetate (3×150 mL). The combined organic layers were washed with brine (200 mL), dried over anhydrous sodium sulfate and concentrated under vacuum. The residue was purified by column chromatography on silica gel using 75% petroleum ether and 25% ethyl acetate as eluent to afford to afford 6-(methylamino)-5-(2H-1,2,3-triazol-2-yl)pyridin-2-yl trifluoromethanesulfonate (500 mg, 68% yield) as a yellow solid. ¹H NMR (300 MHz, DMSO-d₆) δ: 8.25 (d, J=8.1 Hz, 1H), 8.22 (m, 2H), 7.87 (br, 1H), 6.74 (d, J=8.4 Hz, 1H), 2.92 (d, J=4.5 Hz, 3H). LC-MS: m/z 324[M+H]⁺.

### Step 7: 6-((diphenylmethylene)amino)-N-methyl-3-(2H-1,2,3-triazol-2-yl)pyridin-2-amine

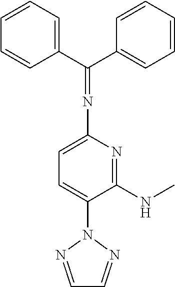 [SMILES: CNc1nc(N=C(c2ccccc2)c2ccccc2)ccc1-n1nccn1]

To a stirred mixture of 6-(methylamino)-5-(2H-1,2,3-triazol-2-yl)pyridin-2-yl trifluoromethanesulfonate (650 mg, 2.0 mmol) and diphenylmethanimine (728 mg, 4.0 mmol) in toluene (30 mL) were added Pd(OAc)₂ (135 mg, 603 μmol), BINAP (392 mg, 603 μmol) and Cs₂CO₃ (1.3 g, 4.0 mmol) under nitrogen atmosphere. The resulting mixture was stirred at 100° C. for 2 h under nitrogen atmosphere. After cooled to 25° C., the reaction mixture was quenched with water (50 mL). The resulting mixture was extracted with ethyl acetate (3×80 mL). The combined organic layers were washed with brine (100 mL), dried over anhydrous sodium sulfate and concentrated under vacuum. The residue was purified by column chromatography on silica gel using 20% petroleum ether and 80% ethyl acetate as eluent to afford 6-((diphenylmethylene)amino)-N-methyl-3-(2H-1,2,3-triazol-2-yl)pyridin-2-amine (610 mg, 68% yield) as a yellow oil. ¹H NMR (300 MHz, DMSO-d₆) δ: 8.09 (s, 2H), 7.81 (d, J=8.1 Hz, 1H), 7.66-7.74 (m, 3H), 7.48-7.60 (m, 3H), 7.34-7.36 (m, 3H), 7.18-7.22 (m, 2H), 6.06 (d, J=8.1 Hz, 1H), 2.75 (d, J=4.8 Hz, 3H). LC-MS: m/z 355[M+H]⁺.

### Step 8: N²-methyl-3-(2H-1,2,3-triazol-2-yl)pyridine-2,6-diamine

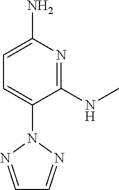

To a stirred mixture of 6-((diphenylmethylene)amino)-N-methyl-3-(2H-1,2,3-triazol-2-yl)pyridin-2-amine (300 mg, 846.5 μmol) in methanol (8 mL) were added hydroxylamine hydrochloride (117 mg, 1.6 mmol) and sodium acetate (173 mg, 2.1 mmol) at 25° C. The resulting mixture was stirred at 25° C. for 1 h. The reaction mixture was concentrated under vacuum. The residue was purified by Prep-TLC using 50% petroleum ether and 50% ethyl acetate as eluent to afford N²-methyl-3-(2H-1,2,3-triazol-2-yl)pyridine-2,6-diamine (120 mg, 74% yield) as a yellow solid. ¹H NMR (400 MHz, DMSO-d₆) δ: 7.97 (s, 2H), 7.57 (d, J=8.4 Hz, 1H), 6.74-6.78 (m, 1H), 5.95 (br, 2H), 5.75 (d, J=8.4 Hz, 1H), 2.85 (d, J=4.4 Hz, 3H). LC-MS: m/z 191 [M+H]⁺.

### Step 9: (R)-2-chloro-8-methyl-N-(6-(methylamino)-5-(2H-1,2,3-triazol-2-yl)pyridin-2-yl)-8-(trifluoromethyl)-7,8-dihydro-6H-pyrazolo[1,5-a]pyrrolo[2,3-e]pyrimidine-6-carboxamide

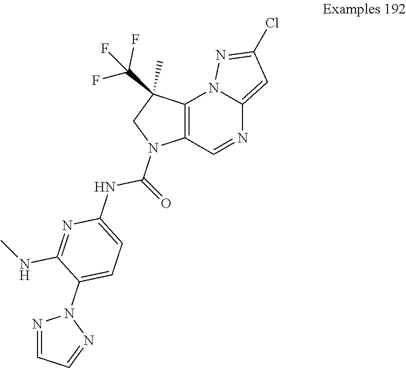 [SMILES: CNc1nc(NC(=O)N2C[C@@](C)(C(F)(F)F)c3c2cnc2cc(Cl)nn32)ccc1-n1nccn1]

To a stirred mixture of N²-methyl-3-(2H-1,2,3-triazol-2-yl)pyridine-2,6-diamine (60 mg, 315.4 μmol) in tetrahydrofuran (5 mL) were added triphosgene (56 mg, 189.2 μmol) and TEA (47 mg, 473.7 μmol). The resulting mixture was stirred at 25° C. for 0.5 h and then filtered. The filtrate was added to a solution of Method M1 isomer 2 (80 mg, 252.3 μmol) in tetrahydrofuran (1 mL). To this solution were added TEA (319 mg, 3.1 mmol) and N,N-dimethylpyridin-4-amine (77 mg, 630.9 μmol). The mixture was stirred at 40° C. for 1 h. The mixture was concentrated under vacuum. The residue was purified by Prep-HPLC and the collected fractions were lyophilized to afford (R)-2-chloro-8-methyl-N-(6-(methylamino)-5-(2H-1,2,3-triazol-2-yl)pyridin-2-yl)-8-(trifluoromethyl)-7,8-dihydro-6H-pyrazolo[1,5-a]pyrrolo[2,3-e]pyrimidine-6-carboxamide (27.8 mg, 17% yield) as a light-yellow solid. The enantiomer of Example 192 can be prepared analogously using Method M1 isomer 1.

Examples 192: ¹H NMR (400 MHz, DMSO-d₆) δ: 9.39 (br, 1H), 9.34 (s, 1H), 8.13 (s, 2H), 8.00 (d, J=8.4 Hz, 1H), 7.26-7.29 (m, 2H), 7.05 (s, 1H), 5.05 (d, J=11.6 Hz, 1H), 4.27 (d, J=11.6 Hz, 1H), 3.01 (d, J=4.8 Hz, 3H), 1.96 (s, 3H). LC-MS: m/z 493 [M+H]⁺.

### Method M6

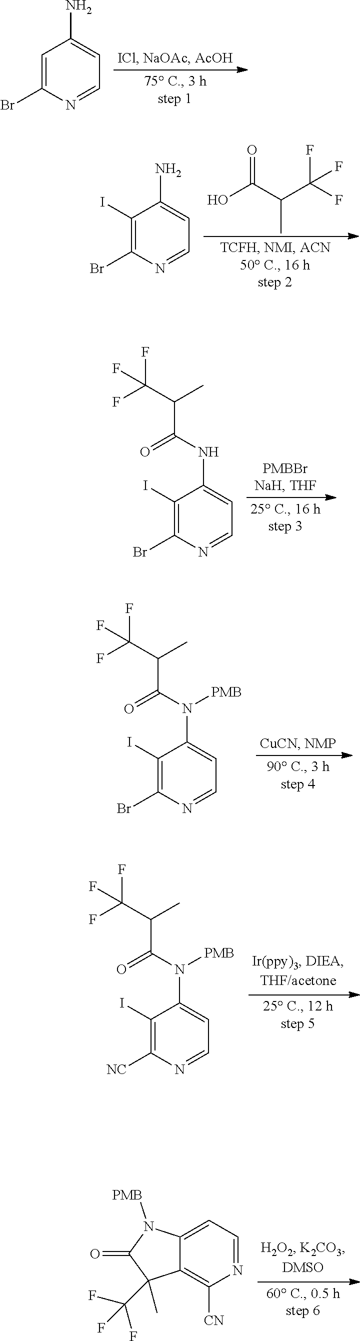

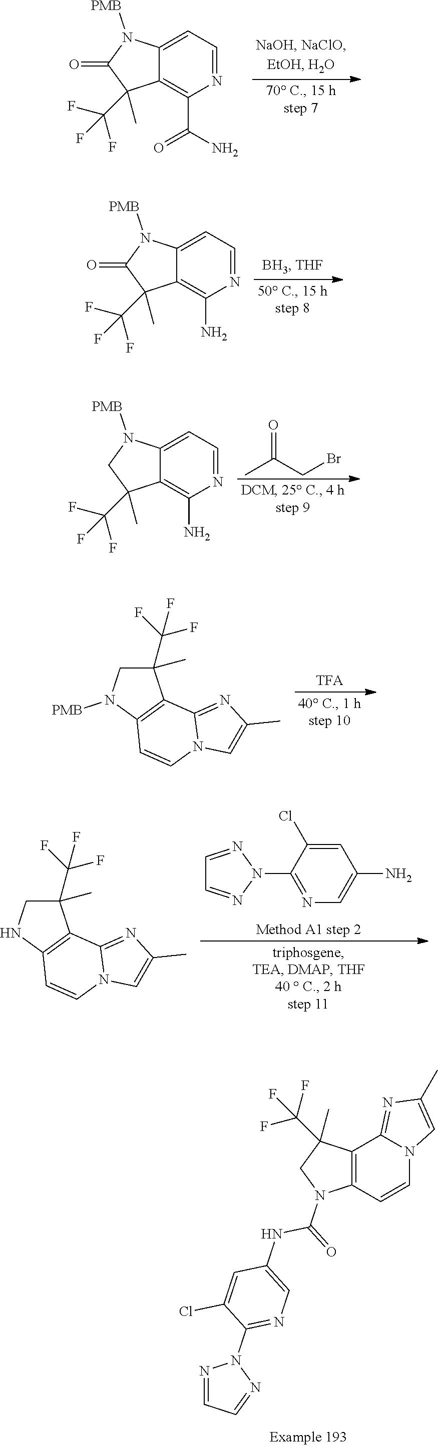

### Example 193: N-(5-chloro-6(2H-1,2,3-triazol-2-yl)pyridin-3-yl)-2,9-dimethyl-9-(trifluoromethyl)-8,9-dihydro-7H-imidazo[1,2-a]pyrrolo[3,2-c]pyridine-7-carboxamide

### Step 1: 2-bromo-3-iodopyridin-4-amine

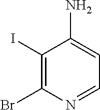

To a stirred solution of 2-bromopyridin-4-amine (50.0 g, 289.0 mmol) and sodium acetate (78.6 g, 578.0 mmol) in acetic acid (160 mL) was added in iodine monochloride (46.9 g, 289.0 mmol). The reaction mixture was stirred at 75° C. for 3 h. The reaction mixture was quenched with water (500 mL). The resulting solution was extracted with ethyl acetate (3×500 mL). The combined organic layers were washed with brine (2×1000 mL), dried over anhydrous sodium sulfate and concentrated under vacuum. The residue was purified by column chromatography on silica gel using 50% petroleum ether and 50% ethyl acetate as eluent to afford 2-bromo-3-iodopyridin-4-amine (28.0 g, 32% yield) as an off-white solid. ¹H NMR (300 MHz, Chloroform-d) δ: 7.90 (d, J=5.4 Hz, 1H), 6.50 (d, J=5.4 Hz, 1H), 4.94 (br, 2H). LC-MS: m/z 299 [M+H]⁺.

### Step 2: N-(2-bromo-3-iodopyridin-4-yl)-3,3,3-trifluoro-2-methylpropanamide

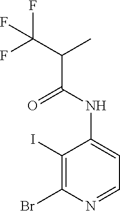 [SMILES: CC(C(=O)Nc1ccnc(Br)c1I)C(F)(F)F]

To a solution of 2-bromo-3-iodo-pyridin-4-amine (20.0 g, 66.9 mmol), N,N,N′,N′-Tetramethylchloroformamidinium hexafluorophosphate (56.3 g, 200.7 mmol) and 1-methylimidazole (27.4 g, 334.5 mmol) in acetonitrile (190 mL) was added 3,3,3-trifluoro-2-methylpropanoic acid (9.5 g, 66.9 mmol). The reaction mixture was stirred at 50° C. for 16 h. The reaction solution was concentrated under vacuum. The residue was quenched with water (500 mL). The resulting solution was extracted with dichloromethane (3×500 mL). The combined organic layers were washed with brine (1000 mL), dried over anhydrous sodium sulfate and concentrated under vacuum. The residue was purified by column chromatography on silica gel using 60% petroleum ether and 40% ethyl acetate as eluent to afford N-(2-bromo-3-iodopyridin-4-yl)-3,3,3-trifluoro-2-methylpropanamide (5.5 g, 19% yield) as a white solid. ¹H NMR (300 MHz, Chloroform-d) δ: 8.22-8.32 (m, 2H), 8.17 (br, 1H), 3.25-3.41 (m, 1H), 1.57 (d, J=7.2 Hz, 3H). LC-MS: m/z 423 [M+H]⁺.

### Step 3: N-(2-bromo-3-iodopyridin-4-yl)-3,3,3-trifluoro-N-(4-methoxybenzyl)-2-methylpropanamide

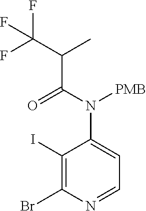

To a stirred solution of N-(2-bromo-3-iodopyridin-4-yl)-3,3,3-trifluoro-2-methylpropanamide (5.0 g, 12.1 mmol) in N,N-Dimethylformamide (100 mL) was added in sodium hydride (520 mg, 13.4 mmol, 60% in mineral oil) in batches at 0° C. The mixture was stirred at 0° C. for 0.5 h. Then 1-(bromomethyl)-4-methoxybenzene (2.8 g, 14 mmol) was added into the reaction mixture. The reaction mixture was stirred at 25° C. for 16 h. The reaction mixture was quenched with ice/water (200 mL). The resulting solution was extracted with ethyl acetate (3×200 mL). The combined organic layers were washed with brine (500 mL), dried over anhydrous sodium sulfate and concentrated under vacuum. The residue was purified by column chromatography on silica gel using 60% petroleum ether and 40% ethyl acetate as eluent to afford N-(2-bromo-3-iodopyridin-4-yl)-3,3,3-trifluoro-N-(4-methoxybenzyl)-2-methylpropanamide (5.2 g, 77% yield) as colorless oil. LC-MS: m/z 543 [M+H]⁺.

### Step 4: N-(2-cyano-3-iodopyridin-4-yl)-3,3,3-trifluoro-N-(4-methoxybenzyl)-2-methylpropanamide

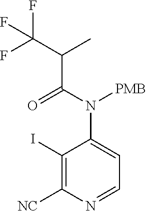

To a stirred solution of N-(2-bromo-3-iodopyridin-4-yl)-3,3,3-trifluoro-N-(4-methoxybenzyl)-2-methylpropanamide (1.5 g, 2.7 mmol) in 1-Methyl-2-pyrrolidinone (30 mL) was added Copper(I) Cyanide (517 mg, 5.5 mmol). The reaction mixture was stirred at 90° C. for 3 h. The reaction mixture was quenched with water (200 mL). The resulting solution was extracted with ethyl acetate (3×200 mL). The combined organic layers were washed with brine (600 mL), dried over anhydrous sodium sulfate and concentrated under vacuum. The residue was purified by column chromatography on silica gel using 60% petroleum ether and 40% ethyl acetate as eluent to afford N-(2-cyano-3-iodopyridin-4-yl)-3,3,3-trifluoro-N-(4-methoxybenzyl)-2-methylpropanamide (1.0 g, 74% yield) as colorless oil. ¹H NMR (300 MHz, Chloroform-d) δ: 8.55-8.57 (m, 1H), 7.03-7.08 (m, 2H), 6.80-6.88 (m, 3H), 5.67 (d, J=14.1 Hz, 1H), 3.96 (d, J=14.4 Hz, 1H), 3.82 (s, 3H), 2.63-2.73 (m, 1H), 1.50 (d, J=6.6 Hz, 3H). LC-MS: m/z 490 [M+H]⁺.

### Step 5: 1-(4-methoxybenzyl)-3-methyl-2-oxo-3-(trifluoromethyl)-2,3-dihydro-1H-pyrrolo[3,2-c]pyridine-4-carbonitrile

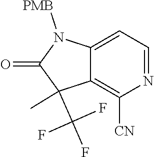

To a stirred solution of N-(2-cyano-3-iodopyridin-4-yl)-3,3,3-trifluoro-N-(4-methoxybenzyl)-2-methylpropanamide (500 mg, 1.1 mmol) in tetrahydrofuran (5 mL) and acetone (5 mL) was added N,N-Diisopropylethylamine (660 mg, 5.1 mmol) and tris(2-phenylpyridine)iridium (6 mg, 10.2 μmol). The reaction mixture was stirred at 25° C. for 12 h irradiated with 450 nm LED. The reaction mixture was concentrated. The residue was purified by column chromatography on silica gel using 50% petroleum ether and 50% ethyl acetate as eluent to afford 1-(4-methoxybenzyl)-3-methyl-2-oxo-3-(trifluoromethyl)-2,3-dihydro-1H-pyrrolo[3,2-c]pyridine-4-carbonitrile (60 mg, 16% yield) as a yellow oil. ¹H NMR (300 MHz, Chloroform-d) S: 8.54 (d, J=5.4 Hz, 1H), 7.18 (d, J=5.7 Hz, 2H), 6.84-6.93 (m, 3H), 5.09 (d, J=15.6 Hz, 1H), 4.74 (d, J=15.3 Hz, 1H), 3.81 (s, 3H), 1.97 (s, 3H). LC-MS: m/z 362 [M+H]⁺.

### Step 6: 1-(4-methoxybenzyl)-3-methyl-2-oxo-3-(trifluoromethyl)-2,3-dihydro-1H-pyrrolo[3,2-c]pyridine-4-carboxamide

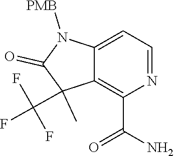

To a stirred mixture of 1-(4-methoxybenzyl)-3-methyl-2-oxo-3-(trifluoromethyl)-2,3-dihydro-1H-pyrrolo[3,2-c]pyridine-4-carbonitrile (670 mg, 1.8 mmol) and K₂CO₃ (512 mg, 3.7 mmol) in dimethyl sulfoxide (10 mL) was added hydrogen peroxide (630 mg, 5.5 mmol, 30% in water). The reaction mixture was stirred at 60° C. for 0.5 h. The reaction mixture was quenched with ice/water (100 mL). The resulting solution was extracted with ethyl acetate (3×100 mL). The combined organic layers were washed with brine (200 mL), dried over anhydrous sodium sulfate and concentrated under vacuum to afford 1-(4-methoxybenzyl)-3-methyl-2-oxo-3-(trifluoromethyl)-2,3-dihydro-1H-pyrrolo[3,2-c]pyridine-4-carboxamide (600 mg, 85% yield) as yellow solid. LC-MS: m/z 380 [M+H]⁺.

### Step 7: 4-amino-1-(4-methoxybenzyl)-3-methyl-3-(trifluoromethyl)-1,3-dihydro-2H-pyrrolo[3,2-c]pyridin-2-one

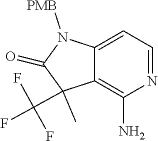

To a stirred mixture of 1-(4-methoxybenzyl)-3-methyl-2-oxo-3-(trifluoromethyl)-2,3-dihydro-1H-pyrrolo[3,2-c]pyridine-4-carboxamide (600 mg, 1.9 mmol) in ethanol (20 mL) and water (10 mL) was added sodium hydroxide (253 mg, 6.3 mmol), and sodium hypochlorite (6.05 g, 7.91 mmol, 10% in water). The reaction mixture was stirred at 70° C. for 15 h. The reaction mixture was concentrated under vacuum to remove ethanol. The resulting solution was extracted with ethyl acetate (3×30 mL). The combined organic layers were washed with brine (50 mL), dried over anhydrous sodium sulfate and concentrated under vacuum. The residue was purified by column chromatography on silica gel using 50% petroleum ether and 50% ethyl acetate as eluent to afford 4-amino-1-(4-methoxybenzyl)-3-methyl-3-(trifluoromethyl)-1,3-dihydro-2H-pyrrolo[3,2-c]pyridin-2-one (400 mg, 72% yield) as yellow solid. ¹H NMR (300 MHz, Chloroform-d) δ: 7.97 (d, J=5.7 Hz, 1H), 7.18 (d, J=8.7 Hz, 2H), 6.88 (d, J=8.7 Hz, 2H), 6.29 (d, J=5.4 Hz, 1H), 5.00 (d, J=15.6 Hz, 1H), 4.97 (br, 2H), 4.69 (d, J=15.6 Hz, 1H), 3.80 (s, 3H), 1.77 (s, 3H). LC-MS: m/z 352 [M+H]⁺.

### Step 8: 1-(4-methoxybenzyl)-3-methyl-3-(trifluoromethyl)-2,3-dihydro-1H-pyrrolo[3,2-c]pyridin-4-amine

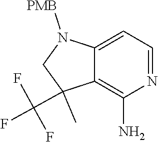

To a stirred solution of 4-amino-1-(4-methoxybenzyl)-3-methyl-3-(trifluoromethyl)-1,3-dihydro-2H-pyrrolo[3,2-c]pyridin-2-one (400 mg, 1.1 mmol) in tetrahydrofuran (10 mL) was added borane (1 N in tetrahydrofuran, 20 mL) at 25° C. The mixture was stirred at 50° C. for 15 h. The reaction mixture was quenched with methanol (50 mL). The resulting solution was concentrated. The residue was dissolved in HCl (20 mL, 1 M). The resulting solution was extracted with ethyl acetate (3×30 mL). The combined organic layers were washed with brine (60 mL), dried over anhydrous sodium sulfate and concentrated under vacuum. The residue was purified by prep-HPLC to afford 1-(4-methoxybenzyl)-3-methyl-3-(trifluoromethyl)-2,3-dihydro-1H-pyrrolo[3,2-c]pyridin-4-amine (160 mg, 41% yield) as yellow oil. LC-MS: m/z 338 [M+H]⁺.

### Step 9: 7-(4-methoxybenzyl)-2,9-dimethyl-9-(trifluoromethyl)-8,9-dihydro-7H-imidazo[1,2-a]pyrrolo[3,2-c]pyridine

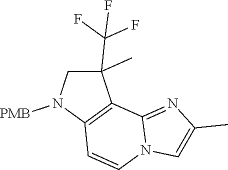

To a stirred solution of 1-(4-methoxybenzyl)-3-methyl-3-(trifluoromethyl)-2,3-dihydro-1H-pyrrolo[3,2-c]pyridin-4-amine (160 mg, 474.3 μmol) in dichloromethane (30 mL) was added 1-bromopropan-2-one (1.95 g, 14.2 mmol) at 25° C. The mixture was stirred at 25° C. for 4 h. The reaction mixture was concentrated under vacuum. The residue was purified by column chromatography on silica gel using 90% ethyl acetate and 10% methanol as eluent to afford 7-(4-methoxybenzyl)-2,9-dimethyl-9-(trifluoromethyl)-8,9-dihydro-7H-imidazo[1,2-a]pyrrolo[3,2-c]pyridine (70 mg, 39% yield) as yellow oil. LC-MS: m/z 376 [M+H]⁺.

### Step 10: 2,9-dimethyl-9-(trifluoromethyl)-8,9-dihydro-7H-imidazo[1,2-a]pyrrolo[3,2-c]pyridine

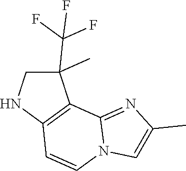 [SMILES: Cc1cn2ccc3c(c2n1)C(C)(C(F)(F)F)CN3]

A mixture of 7-(4-methoxybenzyl)-2,9-dimethyl-9-(trifluoromethyl)-8,9-dihydro-7H-imidazo[1,2-a]pyrrolo[3,2-c]pyridine (70 mg, 186.4 μmol) in TFA (5 mL) was stirred at 40° C. for 1 h. The reaction mixture was concentrated. The residue was purified by prep-HPLC. The collected fractions were combined and concentrated under vacuum to afford 2,9-dimethyl-9-(trifluoromethyl)-8,9-dihydro-7H-imidazo[1,2-a]pyrrolo[3,2-c]pyridine (15 mg, 31% yield) as off-white solid. LC-MS: m/z 256 [M+H]⁺.

### Step 11: N-(5-chloro-6-(2H-1,2,3-triazol-2-yl)pyridin-3-yl)-2,9-dimethyl-9-(trifluoromethyl)-8,9-dihydro-7H-imidazo[1,2-a]pyrrolo[3,2-c]pyridine-7-carboxamide

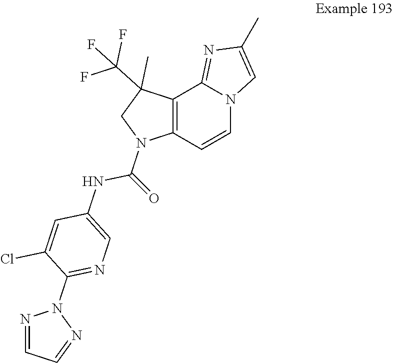 [SMILES: Cc1cn2ccc3c(c2n1)C(C)(C(F)(F)F)CN3C(=O)Nc1cnc(-n2nccn2)c(Cl)c1]

To a stirred solution of 5-chloro-6-(triazol-2-yl)pyridin-3-amine (Method A1 step 2; 14 mg, 70.5 μmol) in tetrahydrofuran (5 mL) were added triphosgene (10 mg, 35.2 μmol) and TEA (9 mg, 88.1 μmol) at 0° C. The resulting mixture was stirred at 25° C. for 0.5 h and then filtered. The filtrate was added to a solution of 2,9-dimethyl-9-(trifluoromethyl)-8,9-dihydro-7H-imidazo[1,2-a]pyrrolo[3,2-c]pyridine (15 mg, 58.7 μmol) in tetrahydrofuran (2 mL). To this solution was then added TEA (59 mg, 587.7 μmol) and N,N-dimethylpyridin-4-amine (14 mg, 117.5 μmol). The mixture was stirred at 40° C. for 2 h. The reaction mixture was quenched with water (10 mL), and extracted with ethyl acetate (3×10 mL). The combined organic layers were washed with brine (20 mL), dried over anhydrous sodium sulfate and concentrated under vacuum. The residue was submitted to Prep-HPLC purification and the collected fractions were lyophilized to give N-(5-chloro-6-(2H-1,2,3-triazol-2-yl)pyridin-3-yl)-2,9-dimethyl-9-(trifluoromethyl)-8,9-dihydro-7H-imidazo[1,2-a]pyrrolo[3,2-c]pyridine-7-carboxamide (11.4 mg, 40% yield) as white solid.

Example 193: ¹H NMR (400 MHz, DMSO-d₆) δ: 9.50 (br, 1H), 8.76 (d, J=2.4 Hz, 1H), 8.49 (d, J=2.4 Hz, 1H), 8.48 (s, 1H), 8.17 (s, 2H), 7.66-7.71 (m, 2H), 4.70 (d, J=11.6 Hz, 1H), 4.19 (d, J=11.6 Hz, 1H), 2.32 (s, 3H), 1.90 (s, 3H). LC-MS: m/z 477 [M+H]⁺.

### Method N6

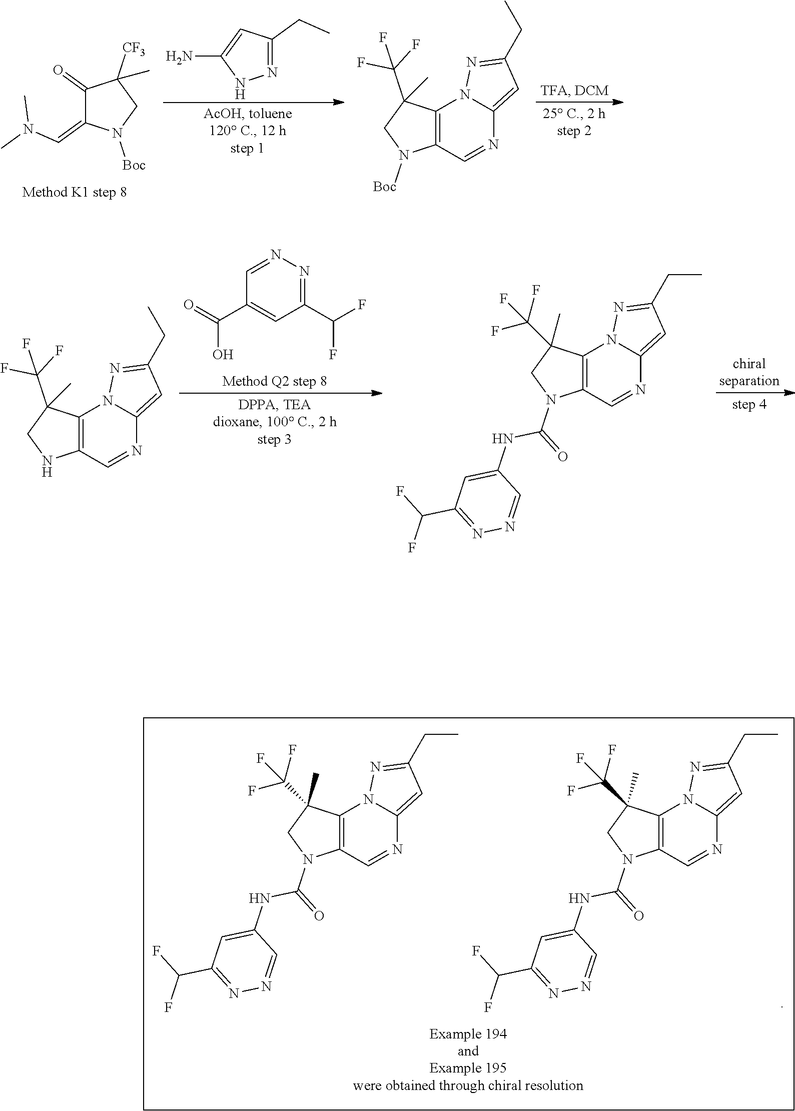

### Examples 194 and 195: Single enantiomers obtained from a racemic mixture containing (S)—N-(6-(difluoromethyl)pyridazin-4-yl)-2-ethyl-8-methyl-8-(trifluoromethyl)-7,8-dihydro-6H-pyrazolo[1,5-a]pyrrolo[2,3-e]pyrimidine-6-carboxamide and (R)—N-(6-(difluoromethyl)pyridazin-4-yl)-2-ethyl-8-methyl-8-(trifluoromethyl)-7,8-dihydro-6H-pyrazolo[1,5-a]pyrrolo[2,3-e]pyrimidine-6-carboxamide

### Step 1: tert-butyl 2-ethyl-8-methyl-8-(trifluoromethyl)-7,8-dihydro-6H-pyrazolo[1,5-a]pyrrolo[2,3-e]pyrimidine-6-carboxylate

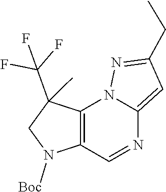 [SMILES: CCc1cc2ncc3c(n2n1)C(C)(C(F)(F)F)CN3C(=O)OC(C)(C)C]

To a stirred solution of 3-ethyl-1H-pyrazol-5-amine (56 mg, 511.9 μmol) in toluene (10 mL) was added acetic acid (1 mL) and tert-butyl (E)-2-((dimethylamino)methylene)-4-methyl-3-oxo-4-(trifluoromethyl)pyrrolidine-1-carboxylate (Method K1 step 8; 165 mg, 511.9 μmol). The reaction mixture was stirred at 120° C. for 12 h. The mixture was concentrated under vacuum. The residue was diluted with saturated aqueous NaHCO₃ solution (10 mL). The resulting solution was extracted with ethyl acetate (3×10 mL). The combined organic layers were dried over anhydrous sodium sulfate and concentrated under vacuum. The residue was purified by column chromatography using 70% petroleum ether and 30% ethyl acetate as eluent to afford tert-butyl 2-ethyl-8-methyl-8-(trifluoromethyl)-7,8-dihydro-6H-pyrazolo[1,5-a]pyrrolo[2,3-e]pyrimidine-6-carboxylate (109 mg, 57% yield) as a white solid. LC-MS: m/z 371 [M+H]⁺.

### Step 2: 2-ethyl-8-methyl-8-(trifluoromethyl)-7,8-dihydro-6H-pyrazolo[1,5-a]pyrrolo[2,3-e]pyrimidine

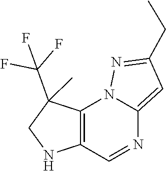 [SMILES: CCc1cc2ncc3c(n2n1)C(C)(C(F)(F)F)CN3]

To a stirred solution of tert-butyl 2-ethyl-8-methyl-8-(trifluoromethyl)-7,8-dihydro-6H-pyrazolo[1,5-a]pyrrolo[2,3-e]pyrimidine-6-carboxylate (102 mg, 275.4 μmol) in dichloromethane (5 mL) was added TFA (1 mL). The reaction mixture was stirred at 25° C. for 2 h. The resulting mixture was concentrated under vacuum. The residue was diluted with saturated aqueous NaHCO₃ solution (10 mL). The resulting solution was extracted with dichloromethane (3×10 mL). The combined organic layers were washed with brine (30 mL), dried over anhydrous sodium sulfate and concentrated under vacuum. The residue was purified by column chromatography on silica gel using 50% petroleum ether and 50% ethyl acetate as eluent to afford 2-ethyl-8-methyl-8-(trifluoromethyl)-7,8-dihydro-6H-pyrazolo[1,5-a]pyrrolo[2,3-e]pyrimidine (60 mg, 81% yield) as a white solid. LC-MS: m/z 271 [M+H]⁺.

### Step 3: N-(6-(difluoromethyl)pyridazin-4-yl)-2-ethyl-8-methyl-8-(trifluoromethyl)-7,8-dihydro-6H-pyrazolo[1,5-a]pyrrolo[2,3-e]pyrimidine-6-carboxamide

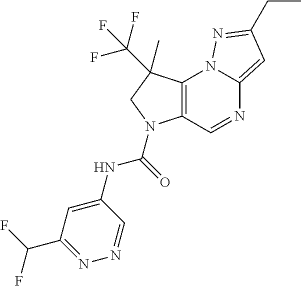 [SMILES: CCc1cc2ncc3c(n2n1)C(C)(C(F)(F)F)CN3C(=O)Nc1cnnc(C(F)F)c1]

To a stirred solution of 2-ethyl-8-methyl-8-(trifluoromethyl)-7,8-dihydro-6H-pyrazolo[1,5-a]pyrrolo[2,3-e]pyrimidine (50 mg, 185 μmol) and 6-(difluoromethyl)pyridazine-4-carboxylic acid (Method Q2 step 8; 48 mg, 277.5 μmol) in dioxane (5 mL) was added DPPA (101 mg, 370 μmol) and TEA (94 mg, 925.1 μmol) at 25° C. The resulting mixture was stirred at 100° C. for 2 h. The reaction was cooled to 25° C. The resulting mixture was concentrated under vacuum. The residue was diluted with water (10 mL), and the resulting solution was extracted with ethyl acetate (3×10 mL). The combined organic layers were dried over anhydrous sodium sulfate and concentrated under vacuum. The residue was purified by Prep-HPLC and the collected fractions were lyophilized to afford N-(6-(difluoromethyl)pyridazin-4-yl)-2-ethyl-8-methyl-8-(trifluoromethyl)-7,8-dihydro-6H-pyrazolo[1,5-a]pyrrolo[2,3-e]pyrimidine-6-carboxamide (32 mg, 35% yield) as a white solid. ¹H NMR (300 MHz, DMSO-d₆) δ: 9.86 (br, 1H), 9.51 (d, J=2.4 Hz, 1H), 9.23 (s, 1H), 8.22 (d, J=2.4 Hz, 1H), 7.24 (t, J=54.3 Hz, 1H), 6.74 (s, 1H), 4.84 (d, J=11.4 Hz, 1H), 4.29 (d, J=11.4 Hz, 1H), 2.84 (q, J=7.8 Hz, 2H), 2.01 (s, 3H), 1.30 (t, J=7.5 Hz, 3H). LC-MS: m/z 442 [M+H]⁺.

### Step 4: Separation of enantiomers to obtain (S)—N-(6-(difluoromethyl)pyridazin-4-yl)-2-ethyl-8-methyl-8-(trifluoromethyl)-7,8-dihydro-6H-pyrazolo[1,5-a]pyrrolo[2,3-e]pyrimidine-6-carboxamide and (R)—N-(6-(difluoromethyl)pyridazin-4-yl)-2-ethyl-8-methyl-8-(trifluoromethyl)-7,8-dihydro-6H-pyrazolo[1,5-a]pyrrolo[2,3-e]pyrimidine-6-carboxamide

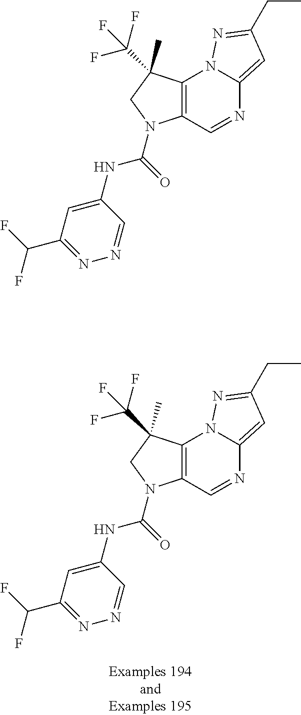 [SMILES: CCc1cc2ncc3c(n2n1)[C@@](C)(C(F)(F)F)CN3C(=O)Nc1cnnc(C(F)F)c1 ; CCc1cc2ncc3c(n2n1)[C@](C)(C(F)(F)F)CN3C(=O)Nc1cnnc(C(F)F)c1]

N-(6-(difluoromethyl)pyridazin-4-yl)-2-ethyl-8-methyl-8-(trifluoromethyl)-7,8-dihydro-6H-pyrazolo[1,5-a]pyrrolo[2,3-e]pyrimidine-6-carboxamide (30 mg, 68.0 μmol) was submitted to chiral-HPLC: Column: CHIRALPAK IA, 2×25 cm, 5 μm; Mobile Phase A:Hex(0.1% FA)-HPLC, Mobile Phase B:EtOH-HPLC; Flow rate: 20 mL/min; Gradient: 5 B to 5 B in 36 min; 220/254 nm; RT1:35.529; RT2:43.483; Injection Volume: 0.7 ml; Number Of Runs: 4. The first eluting isomer was concentrated and lyophilized to afford Example 194 (2.3 mg, 3% yield) as a white solid. The second eluting isomer was concentrated and lyophilized to afford Example 195 (2.4 mg, 3% yield) as a white solid. Examples 194 and 195 are enantiomers, but their absolute stereochemistry is not yet known.

Example 194: ¹H NMR (400 MHz, DMSO-d₆) δ: 9.43 (s, 1H), 9.26 (s, 1H), 8.20 (s, 1H), 7.20 (t, J=54.4 Hz, 1H), 6.72 (s, 1H), 4.82 (d, J=11.6 Hz, 1H), 4.27 (d, J=11.6 Hz, 1H), 2.83 (q, J=7.6 Hz, 2H), 2.00 (s, 3H), 1.30 (t, J=7.6 Hz, 3H). LC-MS: m/z 442 [M+H]⁺.

Example 195: ¹H NMR (400 MHz, DMSO-d₆) δ: 9.42 (s, 1H), 9.26 (s, 1H), 8.20 (s, 1H), 7.19 (t, J=54.8 Hz, 1H), 6.72 (s, 1H), 4.82 (d, J=11.6 Hz, 1H), 4.27 (d, J=11.6 Hz, 1H), 2.81 (q, J=7.2 Hz, 2H), 2.00 (s, 3H), 1.30 (t, J=7.6 Hz, 3H). LC-MS: m/z 442 [M+H]⁺.

### Method O6

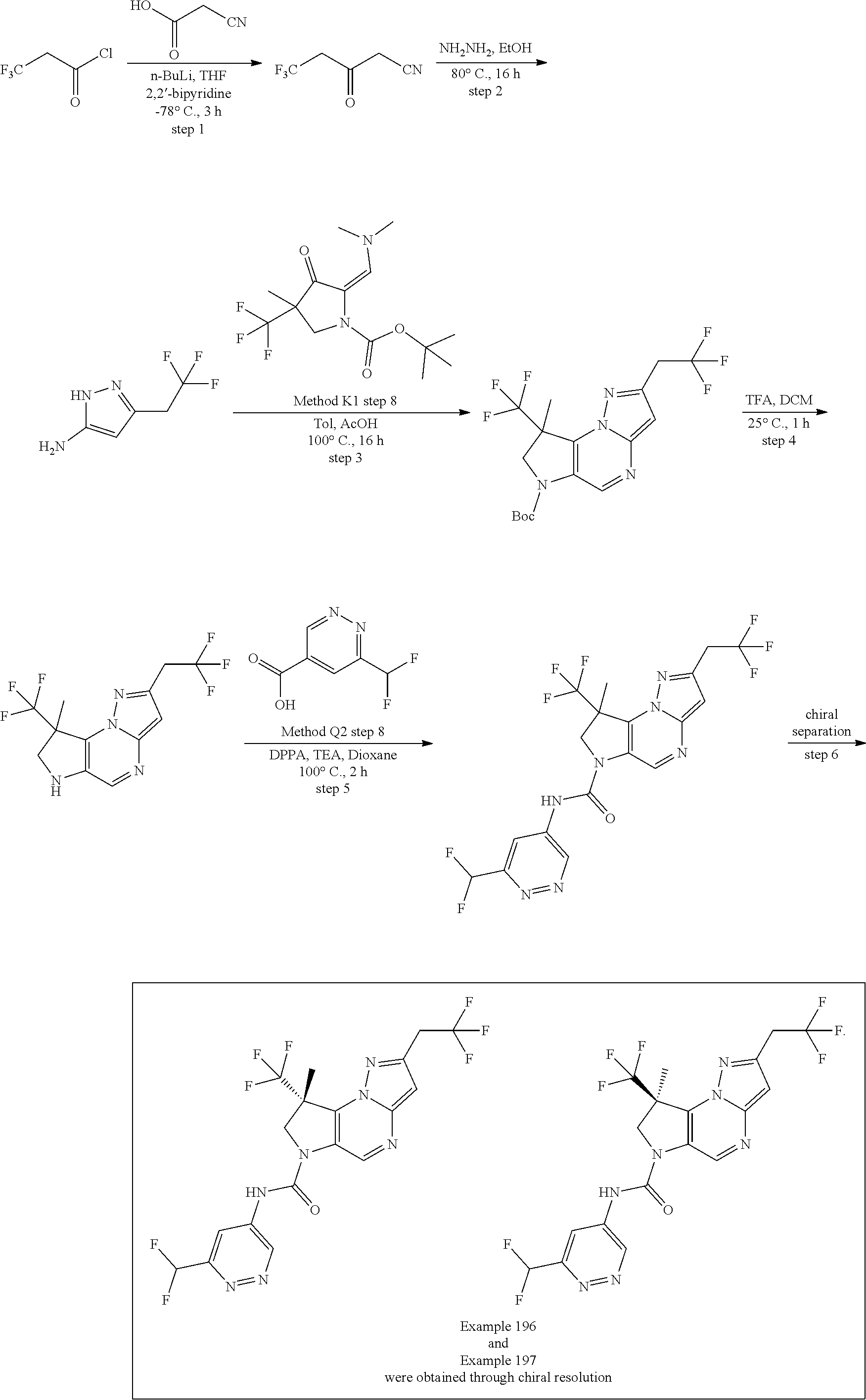

### Examples 1% and 197: Single enantiomers obtained from a racemic mixture containing (S)—N-(6-(difluoromethyl)pyridazin-4-yl)-8-methyl-2-(2,2,2-trifluoroethyl)-8-(trifluoromethyl)-7,8-dihydro-6H-pyrazolo[1,5-a]pyrrolo[2,3-e]pyrimidine-6-carboxamide and (R)—N-(6-(difluoromethyl)pyridazin-4-yl)-8-methyl-2-(2,2,2-trifluoroethyl)-8-(trifluoromethyl)-7,8-dihydro-6H-pyrazolo[1,5-a]pyrrolo[2,3-e]pyrimidine-6-carboxamide

### Step 1: 5,5,5-trifluoro-3-oxopentanenitrile

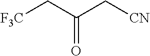

To a stirred solution of 2-cyanoacetic acid (3.5 g, 40.9 mmol) and 2,2′-bipyridine (32 mg, 204.8 μmol) in tetrahydrofuran (120 mL) was added n-BuLi (32.8 mL, 82.0 mmol, 2.5 M in tetrahydrofuran) dropwise at −78° C. under nitrogen atmosphere. The reaction mixture was stirred at −10° C. for 15 min. To this mixture was then added 3,3,3-trifluoropropanoyl chloride (3.1 g, 20.4 mmol) dropwise at −78° C. The reaction mixture was stirred at −78° C. for 3 h. The reaction mixture was quenched with water (400 mL) at −78° C. To this solution was added HCl (50 mL, 1 M). The resulting solution was extracted with ethyl acetate (3×500 mL). The combined organic layers were dried over anhydrous sodium sulfate and concentrated under vacuum. The residue was purified by column chromatography on silica gel using 83% petroleum ether and 17% ethyl acetate as eluent to afford 5,5,5-trifluoro-3-oxopentanenitrile (2.2 g, 67% yield) as a red solid. ¹H NMR (300 MHz, Chloroform-d) δ: 3.66 (s, 2H), 3.48 (q, J=9.9 Hz, 2H). LC-MS: m/z 152 [M+H]⁺.

### Step 2: 3-(2,2,2-trifluoroethyl)-1H-pyrazol-5-amine

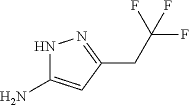 [SMILES: Nc1cc(CC(F)(F)F)n[nH]1]

To a stirred solution of 5,5,5-trifluoro-3-oxopentanenitrile (500 mg, 3.3 mmol) in ethanol (10 mL) was added hydrazine hydrate (497 mg, 9.9 mmol). The reaction mixture was stirred at 80° C. for 16 h. The mixture was cooled to 25° C. The resulting mixture was concentrated under vacuum. The residue was purified by column chromatography on silica gel using 95% dichloromethane and 5% methanol as eluent to afford 3-(2,2,2-trifluoroethyl)-1H-pyrazol-5-amine (260 mg, 43% yield) as a light yellow solid. LC-MS: m/z 166 [M+H]⁺.

### Step 3: tert-butyl 8-methyl-2-(2,2,2-trifluoroethyl)-8-(trifluoromethyl)-7,8-dihydro-6H-pyrazolo[1,5-a]pyrrolo[2,3-e]pyrimidine-6-carboxylate

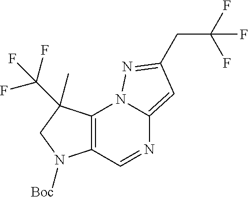 [SMILES: CC(C)(C)OC(=O)N1CC(C)(C(F)(F)F)c2c1cnc1cc(CC(F)(F)F)nn21]

To a stirred solution of 3-(2,2,2-trifluoroethyl)-1H-pyrazol-5-amine (200 mg, 1.2 mmol) in toluene (10 mL) were added acetic acid (1 mL) and tert-butyl (E)-2-((dimethylamino)methylene)-4-methyl-3-oxo-4-(trifluoromethyl)pyrrolidine-1-carboxylate (Method K1 step 8; 558 mg, 1.2 mmol). The reaction mixture was stirred at 100° C. for 16 h. The mixture was cooled to 25° C. The resulting mixture was concentrated under vacuum. The residue was diluted with saturated aqueous NaHCO₃ solution (40 mL). The resulting solution was extracted with ethyl acetate (3×40 mL). The combined organic layers were dried over anhydrous sodium sulfate and concentrated under vacuum. The residue was purified by column chromatography on silica gel using 90% petroleum ether and 10% ethyl acetate as eluent to afford tert-butyl 8-methyl-2-(2,2,2-trifluoroethyl)-8-(trifluoromethyl)-7,8-dihydro-6H-pyrazolo[1,5-a]pyrrolo[2,3-e]pyrimidine-6-carboxylate (380 mg, 70% yield) as a light yellow solid. LC-MS: m/z 425 [M+H]⁺.

### Step 4: 8-methyl-2-(2,2,2-trifluoroethyl)-8-(trifluoromethyl)-7,8-dihydro-6H-pyrazolo[1,5-a]pyrrolo[2,3-e]pyrimidine

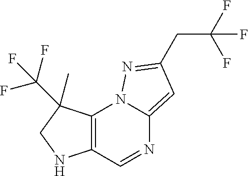 [SMILES: CC1(C(F)(F)F)CNc2cnc3cc(CC(F)(F)F)nn3c21]

To a stirred solution of tert-butyl 8-methyl-2-(2,2,2-trifluoroethyl)-8-(trifluoromethyl)-7,8-dihydro-6H-pyrazolo[1,5-a]pyrrolo[2,3-e]pyrimidine-6-carboxylate (300 mg, 706.9 μmol) in dichloromethane (6 mL) was added TFA (2.5 mL). The reaction mixture was stirred at 25° C. for 1 h. The resulting solution was concentrated under vacuum. The residue was diluted with saturated aqueous NaHCO₃ solution (40 mL). The resulting solution was extracted with dichloromethane (3×40 mL). The combined organic layers were dried over anhydrous sodium sulfate and concentrated under vacuum. The residue was purified by column chromatography on silica gel using 99% dichloromethane and 1% methanol as eluent to afford 8-methyl-2-(2,2,2-trifluoroethyl)-8-(trifluoromethyl)-7,8-dihydro-6H-pyrazolo[1,5-a]pyrrolo[2,3-e]pyrimidine (220 mg, 91% yield) as a yellow oil. ¹H NMR (300 MHz, DMSO-d₆) δ: 8.35 (s, 1H), 6.72 (s, 1H), 5.91 (br, 1H), 3.81-3.92 (m, 3H), 3.58 (d, J=11.7 Hz, 1H), 1.84 (s, 3H). LC-MS: m/z 325 [M+H]⁺.

### Step 5: N-(6-(difluoromethyl)pyridazin-4-yl)-8-methyl-2-(2,2,2-trifluoroethyl)-8-(trifluoromethyl)-7,8-dihydro-6H-pyrazolo[1,5-a]pyrrolo[2,3-e]pyrimidine-6-carboxamide

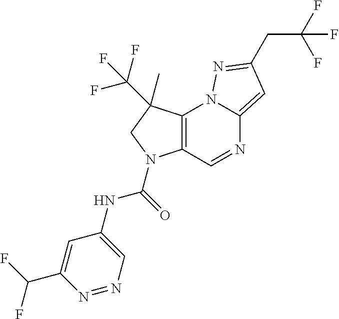 [SMILES: CC1(C(F)(F)F)CN(C(=O)Nc2cnnc(C(F)F)c2)c2cnc3cc(CC(F)(F)F)nn3c21]

To a stirred solution of 8-methyl-2-(2,2,2-trifluoroethyl)-8-(trifluoromethyl)-7,8-dihydro-6H-pyrazolo[1,5-a]pyrrolo[2,3-e]pyrimidine in dioxane (6 ML) was added 6-(difluoromethyl)pyridazine-4-carboxylic acid (Method Q2 step 8; 54 mg, 308.4 μmol), TEA (156 mg, 1.5 mmol) and DPPA (102 mg, 370 μmol) at 25° C. The reaction mixture was stirred at 100° C. for 2 h. The reaction mixture was cooled to 25° C., and the mixture was concentrated under vacuum. The residue was purified by Prep-TLC using 95% dichloromethane and 5% methanol as eluent to afford the crude product. The crude product was submitted to Prep-HPLC purification and the collected fractions were lyophilized to afford N-(6-(difluoromethyl)pyridazin-4-yl)-8-methyl-2-(2,2,2-trifluoroethyl)-8-(trifluoromethyl)-7,8-dihydro-6H-pyrazolo[1,5-a]pyrrolo[2,3-e]pyrimidine-6-carboxamide (60 mg, 39% yield) as a white solid. ¹H NMR (400 MHz, DMSO-&) δ: 9.90 (br, 1H), 9.50 (d, J=2.4 Hz, 1H), 9.33 (s, 1H), 8.22 (d, J=2.4 Hz, 1H), 7.24 (t, J=54.4 Hz, 1H), 6.92 (s, 1H), 4.86 (d, J=11.6 Hz, 1H), 4.31 (d, J=11.6 Hz, 1H), 3.97 (q, J=11.2 Hz, 2H), 2.01 (s, 3H). LC-MS: m/z 496 [M+H]⁺.

### Step 6: Separation of enantiomers to obtain (S)—N-(6-(difluoromethyl)pyridazin-4-yl)-8-methyl-2-(2,2,2-trifluoroethyl)-8-(trifluoromethyl)-7,8-dihydro-6H-pyrazolo[1,5-a]pyrrolo[2,3-e]pyrimidine-6-carboxamide and (R)—N-(6-(difluoromethyl)pyridazin-4-yl)-8-methyl-2-(2,2,2-trifluoroethyl)-8-(trifluoromethyl)-7,8-dihydro-6H-pyrazolo[1,5-a]pyrrolo[2,3-e]pyrimidine-6-carboxamide

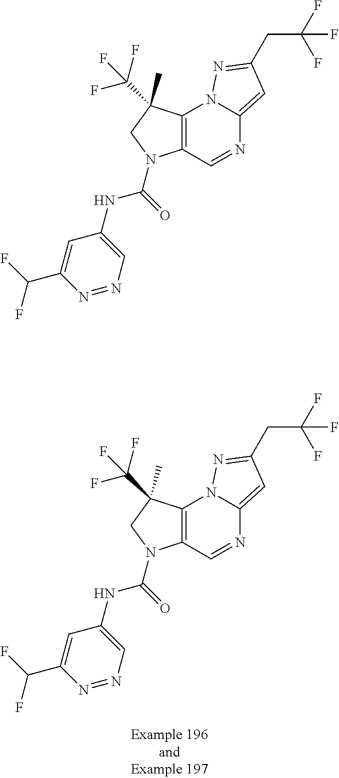 [SMILES: C[C@]1(C(F)(F)F)CN(C(=O)Nc2cnnc(C(F)F)c2)c2cnc3cc(CC(F)(F)F)nn3c21 ; C[C@@]1(C(F)(F)F)CN(C(=O)Nc2cnnc(C(F)F)c2)c2cnc3cc(CC(F)(F)F)nn3c21]

N-(6-(difluoromethyl)pyridazin-4-yl)-8-methyl-2-(2,2,2-trifluoroethyl)-8-(trifluoromethyl)-7,8-dihydro-6H-pyrazolo[1,5-a]pyrrolo[2,3-e]pyrimidine-6-carboxamide (50 mg, 100.9 μmol) was submitted to chiral-HPLC purification: Column: CHIRALPAK IH, 2×25 cm, 5 μm; Mobile Phase A: Hex (0.5% 2M NH₃-MeOH)—HPLC, Mobile Phase B: EtOH—HPLC; Flow rate: 20 mL/min; Gradient: 20 B to 20 B in 19 min; 220/254 nm; RT1: 10.496; RT2: 12.863; Injection Volume: 0.5 ml; Number Of Runs: 5. The first eluting isomer was concentrated and lyophilized to afford Example 1% (11 mg, 23% yield) as a white solid. The second eluting isomer was concentrated and lyophilized to afford Example 197 (15 mg, 30% yield) as a white solid. Examples 196 and 197 are enantiomers, but their absolute stereochemistry is not yet known.

Example 196: ¹H NMR (300 MHz, DMSO-d₆) δ: 9.91 (br, 1H), 9.52 (d, J=2.4 Hz, 1H), 9.33 (s, 1H), 8.23 (d, J=2.4 Hz, 1H), 7.25 (t, J=54.3 Hz, 1H), 6.92 (s, 1H), 4.87 (d, J=11.7 Hz, 1H), 4.32 (d, J=11.7 Hz, 1H), 3.97 (q, J=11.4 Hz, 2H), 2.02 (s, 3H). LC-MS: m/z 4% [M+H]⁺.

Example 197: ¹H NMR (300 MHz, DMSO-d₆) δ: 9.91 (br, 1H), 9.52 (d, J=2.4 Hz, 1H), 9.33 (s, 1H), 8.23 (d, J=2.4 Hz, 1H), 7.25 (t, J=54.3 Hz, 1H), 6.92 (s, 1H), 4.87 (d, J=11.4 Hz, 1H), 4.32 (d, J=11.4 Hz, 1H), 3.97 (q, J=11.4 Hz, 2H), 2.02 (s, 3H). LC-MS: m/z 4% [M+H]⁺.

### Method P6

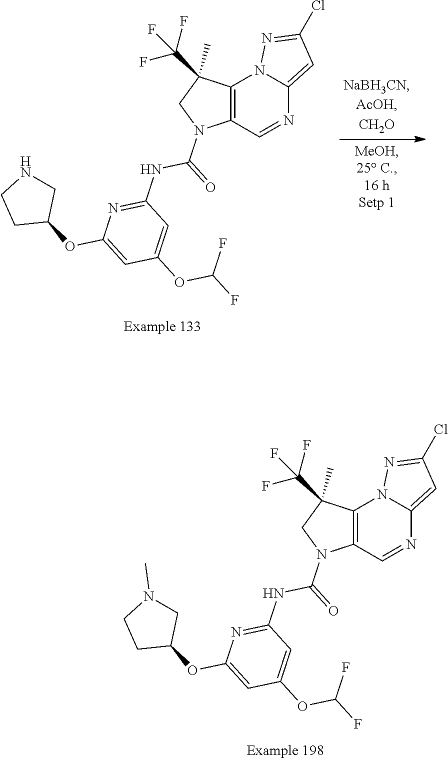 [SMILES: C[C@@]1(C(F)(F)F)CN(C(=O)Nc2cc(OC(F)F)cc(O[C@H]3CCNC3)n2)c2cnc3cc(Cl)nn3c21 ; CN1CC[C@H](Oc2cc(OC(F)F)cc(NC(=O)N3C[C@@](C)(C(F)(F)F)c4c3cnc3cc(Cl)nn43)n2)C1]

### Example 198: (R)-2-chloro-N-(4-(difluoromethoxy)-6-(((S)-1-methylpyrrolidin-3-yl)oxy)pyridin-2-yl)-8-methyl-8-(trifluoromethyl)-7,8-dihydro-6H-pyrazolo[1,5-a]pyrrolo[2,3-e]pyrimidine-6-carboxamide

### Step 1: (R)-2-chloro-N-(4-(difluoromethoxy)-6-(((S)-1-methylpyrrolidin-3-yl)oxy)pyridin-2-yl)-8-methyl-8-(trifluoromethyl)-7,8-dihydro-6H-pyrazolo[1,5-a]pyrrolo[2,3-e]pyrimidine-6-carboxamide

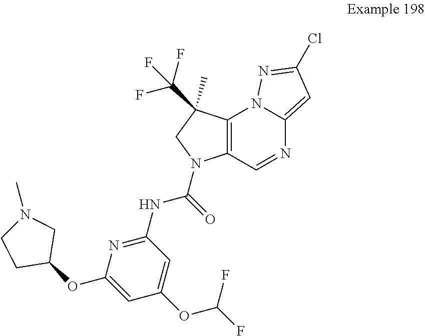 [SMILES: CN1CC[C@H](Oc2cc(OC(F)F)cc(NC(=O)N3C[C@@](C)(C(F)(F)F)c4c3cnc3cc(Cl)nn43)n2)C1]

To a stirred solution of (R)-2-chloro-N-(4-(difluoromethoxy)-6-(((S)-pyrrolidin-3-yl)oxy)pyridin-2-yl)-8-methyl-8-(trifluoromethyl)-7,8-dihydro-6H-pyrazolo[1,5-a]pyrrolo[2,3-e]pyrimidine-6-carboxamide (Example 133; 9 mg, 16.4 μmol) in methanol (0.5 mL) was added acetic acid (5 μL) and formaldehyde (4 μL, 19 μmol, 40% in water) at 0° C. The mixture was stirred at 0° C. for 10 min. Then sodium cyanotrihydroborate (3 mg, 41 μmol) was added at 0° C. The mixture was stirred at 25° C. for 16 h. The mixture was concentrated under vacuum. The residue was purified by Prep-HPLC purification and the collected fractions were lyophilized to give (R)-2-chloro-N-(4-(difluoromethoxy)-6-(((S)-1-methylpyrrolidin-3-yl)oxy)pyridin-2-yl)-8-methyl-8-(trifluoromethyl)-7,8-dihydro-6H-pyrazolo[1,5-a]pyrrolo[2,3-e]pyrimidine-6-carboxamide (4.1 mg, 44% yield) as a white solid. The corresponding epimer of Example 198 with respect to the chiral center attached to the trifluoromethyl group can be prepared analogously using the epimer of Example 133.

Example 198: ¹H NMR (400 MHz, Chloroform-d) δ: 9.41 (s, 1H), 7.51 (d, J=1.2 Hz, 1H), 6.76 (s, 1H), 6.66 (t, J=72.4 Hz, 1H), 6.21 (d, J=1.6 Hz, 1H), 5.53 (s, 1H), 4.67 (d, J=10.8 Hz, 1H), 4.11 (d, J=10.8 Hz, 1H), 3.30-3.77 (m, 1H), 2.91-3.32 (m, 3H), 2.71 (s, 3H), 2.43-2.54 (m, 1H), 2.06-2.23 (m, 1H), 2.08 (s, 3H). LC-MS: m/z 562 [M+H]⁺.

### Method Q6

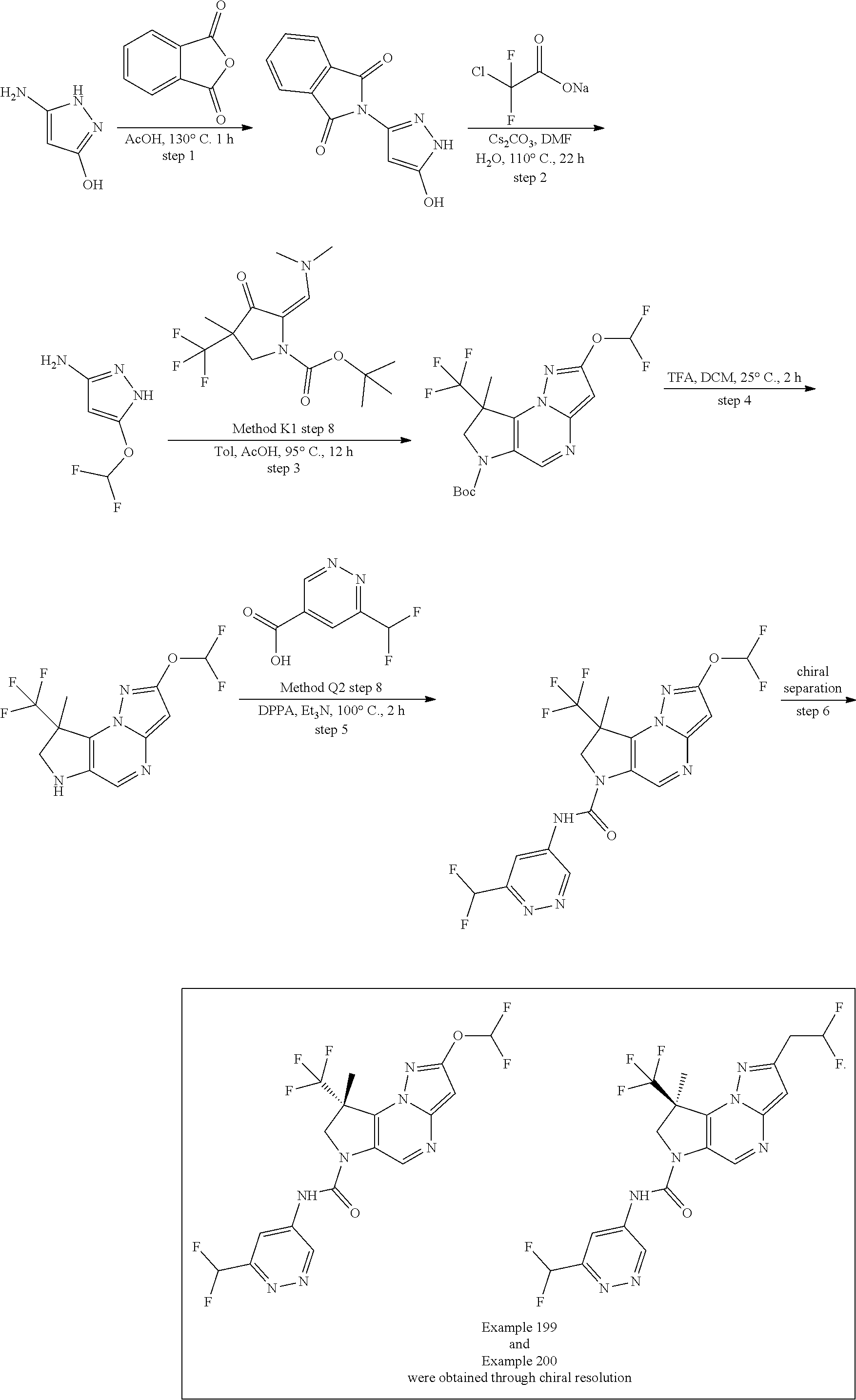

### Examples 199 and 200: Single enantiomers obtained from a racemic mixture containing (S)-2-(difluoromethoxy)-N-(6-(difluoromethyl)pyridazin-4-yl)-8-methyl-8-(trifluoromethyl)-7,8-dihyd O-6H-pyrazolo[1,5-a]pyrrolo[2,3-e]pyrimidine-6-carboxamide and (R)-2-(difluoromethoxy)-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide

### Step 1: 2-(5-hydroxy-1H-pyrazol-3-yl)isoindoline-1,3-dione

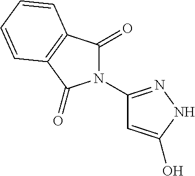 [SMILES: O=C1c2ccccc2C(=O)N1c1cc(O)[nH]n1]

To a stirred mixture of 5-amino-1H-pyrazol-3-ol (10.0 g, 100.9 mmol) in acetic acid (250 mL) was added isobenzofuran-1,3-dione (15.0 g, 101.3 mmol) in portions at 25° C. The resulting mixture was stirred at 130° C. for 1 h. The reaction was cooled to 25° C. The precipitated solid was collected to give 2-(5-hydroxy-1H-pyrazol-3-yl)isoindoline-1,3-dione (18.6 g, 82% yield) as a yellow solid. LC-MS: m/z 230 [M+H]⁺.

### Step 2: 5-(difluoromethoxy)-1H-pyrazol-3-amine

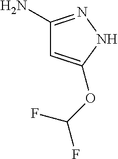

To a stirred solution of 2-(5-hydroxy-1H-pyrazol-3-yl)isoindoline-1,3-dione (9 g, 39.3 mmol) in N,N-Dimethylformamide (90 mL) and water (20 mL) were added Cs₂CO₃ (25.6 g, 78.5 mmol) and sodium 2-chloro-2,2-difluoroacetate (14.1 g, 92.5 mmol) at 25° C. The resulting mixture was stirred at 110° C. for 22 h. The reaction mixture was quenched with saturated aqueous NaHCO₃ solution (100 mL). The resulting mixture was extracted with dichloromethane (3×100 mL). The combined organic layers were washed with brine (100 mL), dried over anhydrous sodium sulfate and concentrated under vacuum. The residue was purified by column chromatography on silica gel using 65% petroleum ether and 35% ethyl acetate as eluent to afford 5-(difluoromethoxy)-1H-pyrazol-3-amine (1.0 g, 6% yield) as a yellow solid. LC-MS: m/z 150 [M+H]⁺.

### Step 3: tert-butyl 2-(difluoromethoxy)-8-methyl-8-(trifluoromethyl)-7,8-dihydro-6H-pyrazolo[1,5-a]pyrrolo[2,3-e]pyrimidine-6-carboxylate

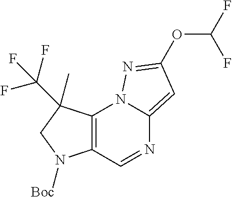 [SMILES: CC(C)(C)OC(=O)N1CC(C)(C(F)(F)F)c2c1cnc1cc(OC(F)F)nn21]

To a stirred solution of 5-(difluoromethoxy)-1H-pyrazol-3-amine (200 mg, 1.3 mmol) in toluene (20 mL) was added acetic acid (2 mL) and tert-butyl (E)-2-((dimethylamino)methylene)-4-methyl-3-oxo-4-(trifluoromethyl)pyrrolidine-1-carboxylate (Method K1 step 8; 418 mg, 1.3 mmol). The reaction mixture was stirred at 95° C. for 12 h. The mixture was concentrated under vacuum. The residue was diluted with saturated aqueous NaHCO₃ solution (20 mL). The resulting solution was extracted with dichloromethane (3×20 mL). The combined organic layers were dried over anhydrous sodium sulfate and concentrated under vacuum. The residue was purified by column chromatography using 65% petroleum ether and 35% ethyl acetate as eluent to afford tert-butyl 2-(difluoromethoxy)-8-methyl-8-(trifluoromethyl)-7,8-dihydro-6H-pyrazolo[1,5-a]pyrrolo[2,3-e]pyrimidine-6-carboxylate (247 mg, 46% yield) as a yellow solid. LC-MS: m/z 409 [M+H]⁺.

### Step 4: 2-(difluoromethoxy)-8-methyl-8-(trifluoromethyl)-7,8-dihydro-6H-pyrazolo[1,5-a]pyrrolo[2,3-e]pyrimidine

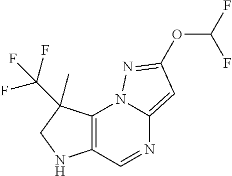 [SMILES: CC1(C(F)(F)F)CNc2cnc3cc(OC(F)F)nn3c21]

To a stirred solution of tert-butyl 2-(difluoromethoxy)-8-methyl-8-(trifluoromethyl)-7,8-dihydro-6H-pyrazolo[1,5-a]pyrrolo[2,3-e]pyrimidine-6-carboxylate (247 mg, 604.9 μmol) in dichloromethane (5 mL) was added TFA (1 mL). The reaction mixture was stirred at 25° C. for 2 h. The resulting mixture was concentrated under vacuum. The residue was diluted with saturated aqueous NaHCO₃ solution (10 mL). The resulting solution was extracted with dichloromethane (3×10 mL). The combined organic layers were washed with brine (30 mL), dried over anhydrous sodium sulfate and concentrated under vacuum to afford 2-(difluoromethoxy)-8-methyl-8-(trifluoromethyl)-7,8-dihydro-6H-pyrazolo[1,5-a]pyrrolo[2,3-e]pyrimidine (187 mg, 92% yield) as a yellow solid. LC-MS: m/z 309 [M+H]⁺.

### Step 5: 2-(difluoromethoxy)-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide

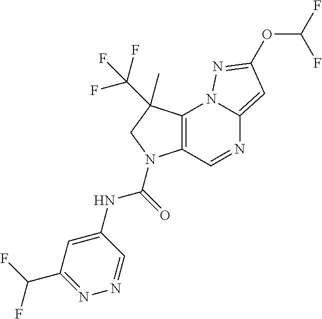 [SMILES: CC1(C(F)(F)F)CN(C(=O)Nc2cnnc(C(F)F)c2)c2cnc3cc(OC(F)F)nn3c21]

To a stirred solution of 2-(difluoromethoxy)-8-methyl-8-(trifluoromethyl)-7,8-dihydro-6H-pyrazolo[1,5-a]pyrrolo[2,3-e]pyrimidine (103 mg, 334.2 μmol) and 6-(difluoromethyl)pyridazine-4-carboxylic acid (Method Q2 step 8; 70 mg, 401.0 μmol) in dioxane (5 mL) was added DPPA (138 mg, 501.3 μmol) and TEA (169 mg, 1.7 mmol) at 25° C. The resulting mixture was stirred at 100° C. for 2 h. The reaction was cooled to 25° C. The resulting mixture was concentrated under vacuum. The residue was diluted with water (10 mL) and extracted with ethyl acetate (3×10 mL). The combined organic layers were dried over anhydrous sodium sulfate and concentrated under vacuum. The residue was purified by Prep-HPLC and the collected fractions were lyophilized to afford 2-(difluoromethoxy)-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide (24 mg, 15% yield) as a white solid. LC-MS: m/z 480 [M+H]⁺.

### Step 6: Separation of enantiomers to obtain (S)-2-(difluoromethoxy)-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide and (R)-2-(difluoromethoxy)-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide

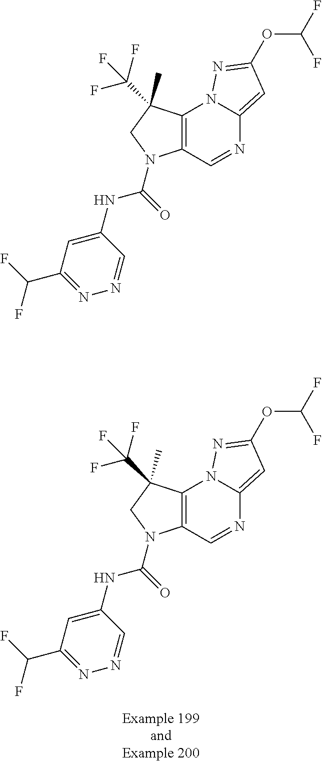 [SMILES: C[C@]1(C(F)(F)F)CN(C(=O)Nc2cnnc(C(F)F)c2)c2cnc3cc(OC(F)F)nn3c21 ; C[C@@]1(C(F)(F)F)CN(C(=O)Nc2cnnc(C(F)F)c2)c2cnc3cc(OC(F)F)nn3c21]

2-(difluoromethoxy)-N-(6-(difluoromethyl)pyridazin-4-yl)-8-methyl-8-(trifluoromethyl)-7,8-dihydro-6H-pyrazolo[1,5-a]pyrrolo[2,3-e]pyrimidine-6-carboxamide (24 mg, 50.0 μmol) was submitted to chiral-HPLC: Column: CHIRAL ART Cellulose-SB, 2*25 cm, 5 μm; Mobile Phase A: Hex(0.5% 2M NH₃-MeOH)-HPLC, Mobile Phase B: EtOH-HPLC; Flow rate: 20 mL/min; Gradient: 20% B to 20% B in 15 min; Wave Length: 220/254 nm; RT1 (min): 10.623; RT2 (min): 13.043; Sample Solvent: EtOH-HPLC; Injection Volume: 1 mL; Number Of Runs: 2. The first eluting isomer was concentrated and lyophilized to afford Example 199 (6.4 mg, 4% yield) as a white solid. The second eluting isomer was concentrated and lyophilized to afford Example 200 (6.1 mg, 4% yield) as a white solid. Examples 199 and 200 are enantiomers, but their absolute stereochemistry is not yet known.

Example 199: ¹H NMR (300 MHz, DMSO-d₆) δ: 9.91 (s, 1H), 9.50 (s, 1H), 9.31 (s, 1H), 8.21 (s, 1H), 7.54 (t, J=72.6 Hz, 1H), 7.24 (t, J=54.0 Hz, 1H), 6.67 (s, 1H), 4.86 (d, J=11.4 Hz, 1H), 4.30 (d, J=11.4 Hz, 1H), 1.98 (s, 3H). LC-MS: m/z 480 [M+H]⁺.

Example 200: ¹H NMR (300 MHz, DMSO-d₆) δ: 9.90 (s, 1H), 9.50 (s, 1H), 9.30 (s, 1H), 8.21 (s, 1H), 7.54 (t, J=72.9 Hz, 1H), 7.24 (t, J=54.0 Hz, 1H), 6.65 (s, 1H), 4.86 (d, J=11.4 Hz, 1H), 4.30 (d, J=11.4 Hz, 1H), 1.97 (s, 3H). LC-MS: m/z 480 [M+H]⁺.

### Method R6

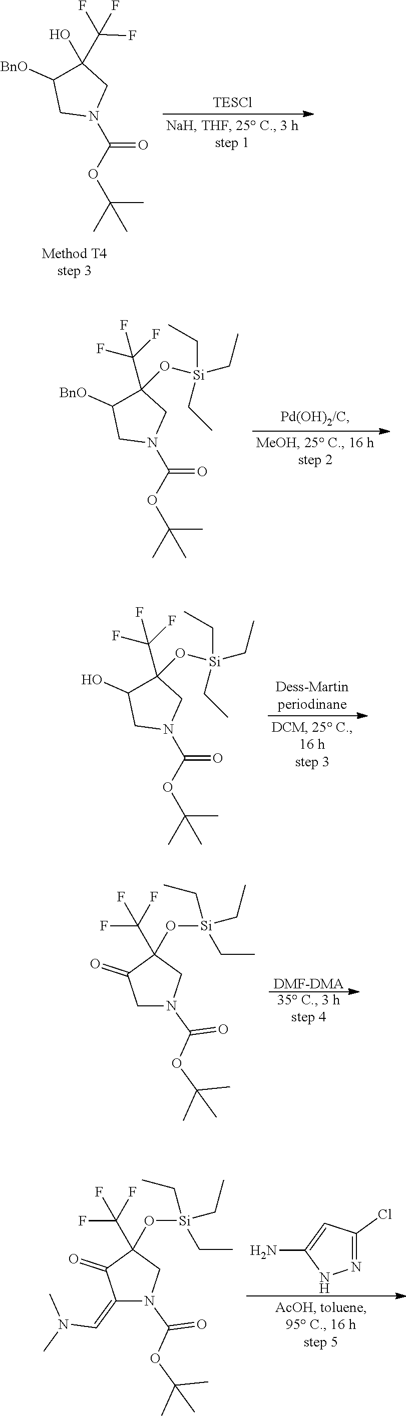

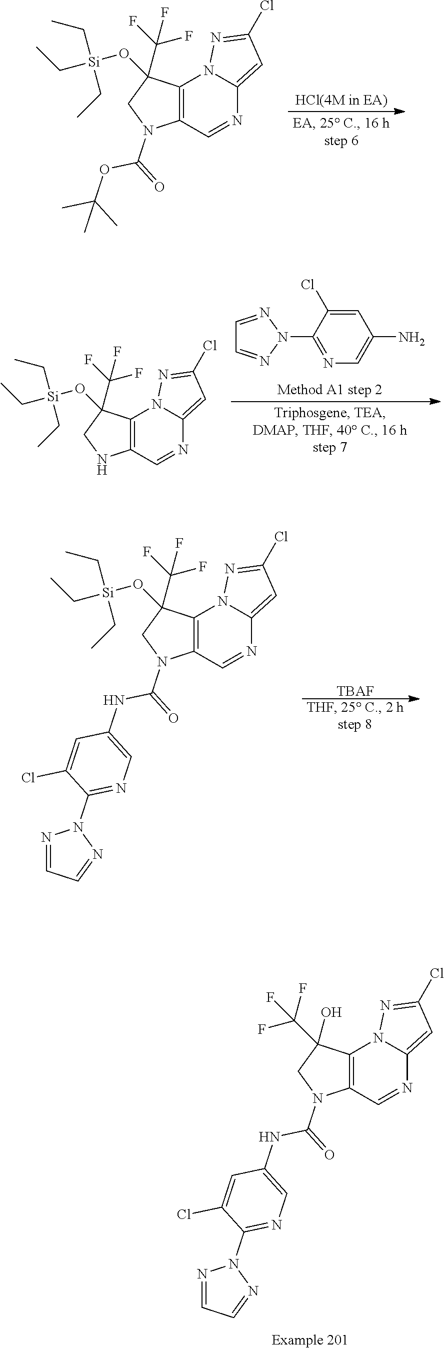

### Examples 201: 2-chloro-N-(5-chloro-6-(2H-1,2,3-triazol-2-yl)pyridin-3-yl)-8-hydroxy-8-(trifluoromethyl)-7,8-dihydro-6H-pyrazolo[1,5-a]pyrrolo[2,3-e]pyrimidine-6-carboxamide

### Step 1: tert-butyl 4-(benzyloxy)-3-((triethylsilyl)oxy)-3-(trifluoromethyl) pyrrolidine-1-carboxylate

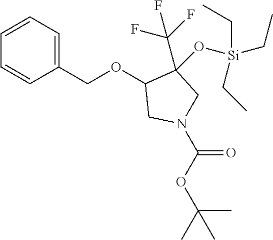 [SMILES: CC[Si](CC)(CC)OC1(C(F)(F)F)CN(C(=O)OC(C)(C)C)CC1OCc1ccccc1]

To a solution of tert-butyl 4-(benzyloxy)-3-hydroxy-3-(trifluoromethyl)pyrrolidine-1-carboxylate (Method T4 step 3; 7.1 g, 19 mmol) in tetrahydrofuran (140 mL) were added NaH (1.5 g, 39 mmol, 60% in mineral oil) at 0° C. The reaction mixture was stirred at 0° C. for 0.5 h. To the mixture was added chlorotriethylsilane (4.4 g, 29 mmol) at 0° C. The resulting mixture was stirred at 25° C. for 3 h. The reaction mixture was quenched by the addition of water (200 mL). The resulting solution was extracted with ethyl acetate (3×200 mL). The combined organic layers were washed with brine (200 mL), dried over anhydrous sodium sulfate and concentrated under vacuum. The residue was purified by column chromatography on silica gel using 70% petroleum ether and 30% ethyl acetate as eluent to afford tert-butyl 4-(benzyloxy)-3-((triethylsilyl)oxy)-3-(trifluoromethyl) pyrrolidine-1-carboxylate (7 g, 59% yield) as a colorless oil. ¹H NMR (400 MHz, DMSO-d₆) δ: 7.24-7.39 (m, 5H), 4.57 (s, 2H), 4.22-4.28 (m, 1H), 3.64-3.69 (m, 1H), 3.55-3.59 (m, 1H), 3.38-3.41 (m, 1H), 3.18-3.23 (m, 1H), 1.37 (s, 9H), 0.86 (t, J=8.0 Hz, 9H), 0.50-0.64 (m, 6H). LC-MS: m/z 476 [M+H]⁺.

### Step 2: tert-butyl 4-hydroxy-3-((triethylsilyl)oxy)-3-(trifluoromethyl) pyrrolidine-1-carboxylate

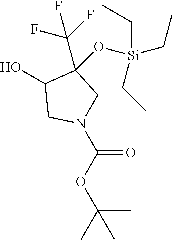 [SMILES: CC[Si](CC)(CC)OC1(C(F)(F)F)CN(C(=O)OC(C)(C)C)CC1O]

To a solution of tert-butyl 4-(benzyloxy)-3-((triethylsilyl)oxy)-3-(trifluoromethyl) pyrrolidine-1-carboxylate (12 g, 25.2 mmol) in methanol (240 mL) was added Pd(OH)₂/C (6 g, 20%). The reaction mixture was stirred at 25° C. for 16 h under hydrogen atmosphere. The solid was filtered out. The filtrate was concentrated under vacuum. The residue was purified by column chromatography on silica gel using 80% petroleum ether and 20% ethyl acetate as eluent to afford 4-hydroxy-3-((triethylsilyl)oxy)-3-(trifluoromethyl) pyrrolidine-1-carboxylate (7 g, 72% yield) as a white solid. LC-MS: m/z 386[M+H]⁺.

### Step 3: tert-butyl 4-oxo-3-((triethylsilyl)oxy)-3-(trifluoromethyl)pyrrolidine-1-carboxylate

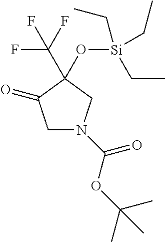 [SMILES: CC[Si](CC)(CC)OC1(C(F)(F)F)CN(C(=O)OC(C)(C)C)CC1=O]

To a solution of tert-butyl 4-hydroxy-3-((triethylsilyl)oxy)-3-(trifluoromethyl) pyrrolidine-1-carboxylate (6 g, 15.5 mmol) in dichloromethane (100 mL) were added Dess-Martin periodinane (33 g, 77.8 mmol). The resulting mixture was stirred at 25° C. for 16 h. The reaction mixture was diluted with saturated aqueous NaHCO₃ solution (200 mL). The resulting solution was extracted with dichloromethane (3×200 mL). The combined organic layers were dried over anhydrous sodium sulfate and concentrated under vacuum. The residue was purified by column chromatography on silica gel using 80% petroleum ether and 20% ethyl acetate as eluent to afford tert-butyl 4-oxo-3-((triethylsilyl)oxy)-3-(trifluoromethyl)pyrrolidine-1-carboxylate (1.6 g, 26% yield) as a yellow oil. LC-MS: m/z 384 [M+H]⁺.

### Step 4: tert-butyl (E)-2-((dimethylamino)methylene)-3-oxo-4-((triethylsilyl)oxy)-4-(trifluoromethyl)pyrrolidine-1-carboxylate

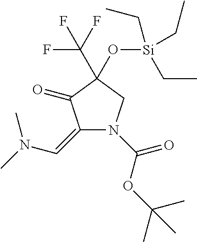 [SMILES: CC[Si](CC)(CC)OC1(C(F)(F)F)CN(C(=O)OC(C)(C)C)/C(=C/N(C)C)C1=O]

A solution of tert-butyl 4-oxo-3-((triethylsilyl)oxy)-3-(trifluoromethyl) pyrrolidine-1-carboxylate (1.6 g, 4.2 mmol) in DMF-DMA (30 mL) was stirred at 35° C. for 3 h. The mixture was cooled to 25° C. The resulting solution was concentrated under vacuum to give tert-butyl (E)-2-((dimethylamino)methylene)-3-oxo-4-((triethylsilyl)oxy)-4-(trifluoromethyl)pyrrolidine-1-carboxylate (1.6 g, crude) as a yellow oil, which was used directly without further purification. LC-MS: m/z 439 [M+H]⁺.

### Step 5: tert-butyl 2-chloro-8-((triethylsilyl)oxy)-8-(trifluoromethyl)-7,8-dihydro-6H-pyrazolo[1,5-a]pyrrolo[2,3-e]pyrimidine-6-carboxylate

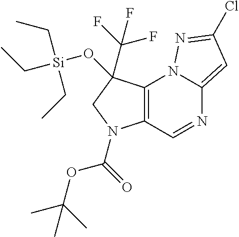 [SMILES: CC[Si](CC)(CC)OC1(C(F)(F)F)CN(C(=O)OC(C)(C)C)c2cnc3cc(Cl)nn3c21]

To a solution of tert-butyl (E)-2-((dimethylamino)methylene)-3-oxo-4-((triethylsilyl)oxy)-4-(trifluoromethyl)pyrrolidine-1-carboxylate (1.6 g, 3.6 mmol) in toluene (30 mL) were added 3-chloro-1H-pyrazol-5-amine (420 mg, 3.6 mmol) and acetic acid (3 mL). The resulting mixture was stirred at 95° C. for 16 h. The mixture was cooled to 25° C. The resulting solution was concentrated under vacuum. The residue was diluted with saturated aqueous NaHCO₃ solution (100 mL). The resulting solution was extracted with ethyl acetate (3×100 mL). The combined organic layers were dried over anhydrous sodium sulfate and concentrated under vacuum. The residue was purified by column chromatography on silica gel using 80% petroleum ether and 20% ethyl acetate as eluent to afford tert-butyl 2-chloro-8-((triethylsilyl)oxy)-8-(trifluoromethyl)-7,8-dihydro-6H-pyrazolo[1,5-a]pyrrolo[2,3-e]pyrimidine-6-carboxylate (200 mg, 11% yield) as a yellow oil. LC-MS: m/z 493 [M+H]⁺.

### Step 6: 2-chloro-8-((triethylsilyl)oxy)-8-(trifluoromethyl)-7,8-dihydro-6H-pyrazolo[1,5-a]pyrrolo[2,3-e]pyrimidine

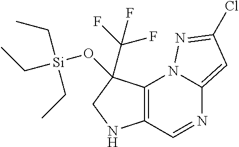 [SMILES: CC[Si](CC)(CC)OC1(C(F)(F)F)CNc2cnc3cc(Cl)nn3c21]

To a solution of tert-butyl 2-chloro-8-((triethylsilyl)oxy)-8-(trifluoromethyl)-7,8-dihydro-6H-pyrazolo[1,5-a]pyrrolo[2,3-e]pyrimidine-6-carboxylate (200 mg, 405 μmol) in ethyl acetate (4 mL) was added HCl (2 mL, 4.0 M in ethyl acetate). The resulting mixture was stirred at 25° C. for 16 h. The mixture was concentrated under vacuum. The residue was diluted with saturated aqueous NaHCO₃ solution (50 mL). The resulting mixture was extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate as eluent to afford 2-chloro-8-((triethylsilyl)oxy)-8-(trifluoromethyl)-7,8-dihydro-6H-pyrazolo[1,5-a]pyrrolo[2,3-e]pyrimidine (66 mg, 41% yield) as a yellow oil. ¹H NMR (300 MHz, Chloroform-d) δ: 8.33 (s, 1H), 6.73 (s, 1H), 4.06-4.14 (m, 1H), 3.70-3.75 (m, 1H), 0.86 (t, J=7.8 Hz, 9H), 0.37-0.46 (m, 6H). LC-MS: m/z 393[M+H]⁺.

### Step 7: 2-chloro-N-(5-chloro-6-(2H-1,2,3-triazol-2-yl)pyridin-3-yl)-8-((triethylsilyl)oxy)-8-(trifluoromethyl)-7,8-dihydro-6H-pyrrazolo[1,5-a]pyrrolo[2,3-e]pyrimidine-6-carboxamide

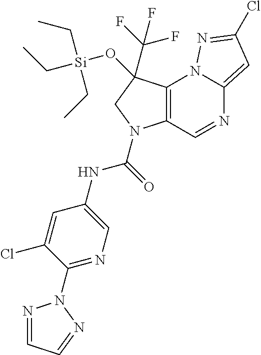 [SMILES: CC[Si](CC)(CC)OC1(C(F)(F)F)CN(C(=O)Nc2cnc(-n3nccn3)c(Cl)c2)c2cnc3cc(Cl)nn3c21]

To a solution of 5-chloro-6-(triazol-2-yl)pyridin-3-amine (Method A1 step 2; 34 mg, 175 μmol) in tetrahydrofuran (3 mL) was added triphosgene (21 mg, 70 μmol) and TEA (18 mg, 175 μmol). The resulting mixture was stirred at 25° C. for 1 h and then filtered. The resulting filtrate was added to a solution of 2-chloro-8-((triethylsilyl)oxy)-8-(trifluoromethyl)-7,8-dihydro-6H-pyrazolo[1,5-a]pyrrolo[2,3-e]pyrimidine (46 mg, 117 μmol) in tetrahydrofuran (1 mL). To this solution was then added TEA (118 mg, 1.2 mmol) and N,N-dimethylpyridin-4-amine (29 mg, 234 μmol). The mixture was stirred at 40° C. for 16 h. The reaction mixture was quenched by the addition of water (20 mL). The resulting solution was extracted with ethyl acetate (3×20 mL). The combined organic layers were dried over anhydrous sodium sulfate and concentrated under vacuum. The residue was purified by column chromatography on silica gel using 10% methanol and 90% dichloromethane as eluent to afford 2-chloro-N-(5-chloro-6-(2H-1,2,3-triazol-2-yl)pyridin-3-yl)-8-((triethylsilyl)oxy)-8-(trifluoromethyl)-7,8-dihydro-6H-pyrazolo[1,5-a]pyrrolo[2,3-e]pyrimidine-6-carboxamide (36 mg, 40% yield) as a white solid. LC-MS: m/z 614 [M+H]⁺.

### Step 8: 2-chloro-N-(5-chloro-6-(2H-1,2,3-triazol-2-yl)pyridin-3-yl)-8-hydroxy-8-(trifluoromethyl)-7,8-dihydro-6H-pyrazolo[1,5-a]pyrrolo[2,3-e]pyrimidine-6-carboxamide

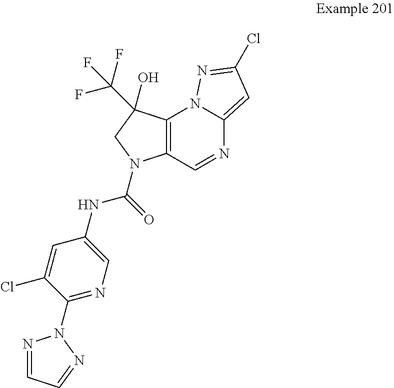 [SMILES: O=C(Nc1cnc(-n2nccn2)c(Cl)c1)N1CC(O)(C(F)(F)F)c2c1cnc1cc(Cl)nn21]

To a stirred mixture of 2-chloro-N-(5-chloro-6-(2H-1,2,3-triazol-2-yl)pyridin-3-yl)-8-((triethylsilyl)oxy)-8-(trifluoromethyl)-7,8-dihydro-6H-pyrazolo[1,5-a]pyrrolo[2,3-e]pyrimidine-6-carboxamide (36 mg, 58 μmol) in tetrahydrofuran (3 mL) was added tetrabutylammonium fluoride (0.29 mL, 290 μmol, 1 M in tetrahydrofuran). The resulting mixture was stirred at 25° C. for 2 h. The reaction mixture was quenched by the addition of water (20 mL). The resulting solution was extracted with ethyl acetate (3×20 mL). The combined organic layers were washed with saturated aqueous NH₄Cl solution (3×20 mL), dried over anhydrous sodium sulfate and concentrated under vacuum. The residue was purified by Prep-HPLC and the collected fractions were lyophilized to afford 2-chloro-N-(5-chloro-6-(2H-1,2,3-triazol-2-yl)pyridin-3-yl)-8-hydroxy-8-(trifluoromethyl)-7,8-dihydro-6H-pyrazolo[1,5-a]pyrrolo[2,3-e]pyrimidine-6-carboxamide (5.3 mg, 18% yield) as a white solid.

Example 201: ¹H NMR (400 MHz, Methanol-d₄) δ: 9.48 (s, 1H), 8.71 (d, J=2.4 Hz, 1H), 8.55 (d, J=2.4 Hz, 1H), 8.02 (s, 2H), 6.84 (s, 1H), 4.81 (d, J=12 Hz, 1H), 4.34-4.38 (m, 1H); LC-MS: m/z 500 [M+H]⁺.

### Method S6

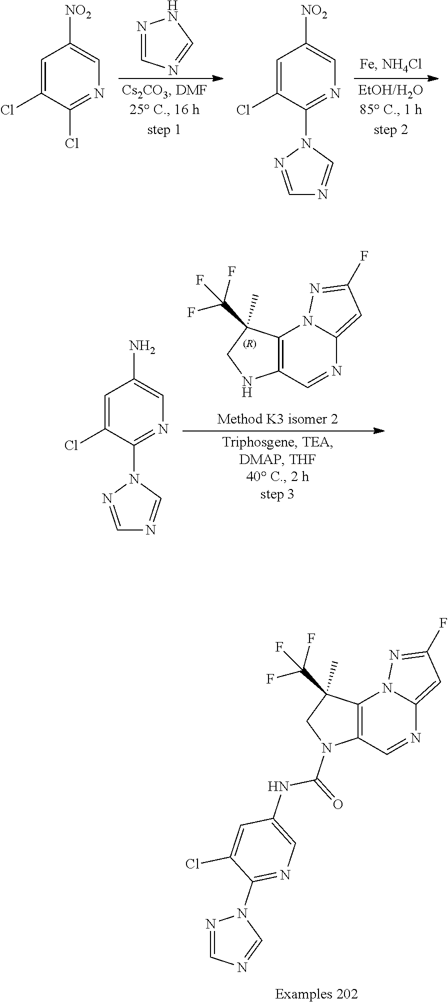

### Examples 202: (R)—N-(5-chloro-6-(1H-1,2,4-triazol-1-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

### Step 1: 3-chloro-5-nitro-2-(1H-1,2,4-triazol-1-yl)pyridine

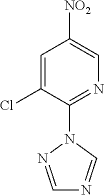 [SMILES: O=[N+]([O-])c1cnc(-n2cncn2)c(Cl)c1]

To a stirred solution of 2,3-dichloro-5-nitropyridine (1.0 g, 5.1 mmol) in N,N-Dimethylformamide (10 mL) was added 1H-1,2,4-triazole (465 mg, 6.7 mmol) and Cs₂CO₃ (3.4 g, 10.4 mmol). The reaction mixture was stirred at 25° C. for 16 h. The reaction mixture was quenched with water (100 mL). The resulting solution was extracted with dichloromethane (3×100 mL). The combined organic layers were washed with brine (300 mL), dried over anhydrous sodium sulfate and concentrated under vacuum. The residue was purified by column chromatography on silica gel using 60% petroleum ether and 40% ethyl acetate as eluent to afford 3-chloro-5-nitro-2-(1H-1,2,4-triazol-1-yl)pyridine (800 mg, 68% yield) as a white solid. ¹H NMR (400 MHz, DMSO-d₆) δ: 9.37 (d, J=2.0 Hz, 1H), 9.28 (s, 1H), 9.13 (d, J=2.0 Hz, 1H), 8.42 (s, 1H).

### Step 2: 5-chloro-6-(1H-1,2,4-triazol-1-yl)pyridin-3-amine

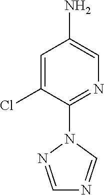 [SMILES: Nc1cnc(-n2cncn2)c(Cl)c1]

To a stirred mixture of 3-chloro-5-nitro-2-(1H-1,2,4-triazol-1-yl)pyridine (300 mg, 1.3 mmol) in ethanol (3 mL) and water (1 mL) were added Fe (148 mg, 2.6 mmol) and NH₄Cl (142 mg, 2.6 mmol) at 25° C. The resulting mixture was stirred at 85° C. for 1 h. The reaction mixture was cooled to 25° C. The reaction mixture was filtered, and the filtrate was concentrated under vacuum. The residue was purified by column chromatography on silica gel using 40% petroleum ether and 60% ethyl acetate as eluent to afford 5-chloro-6-(1H-1,2,4-triazol-1-yl)pyridin-3-amine (120 mg, 46% yield) as a yellow solid. LC-MS: m/z 196 [M+H]⁺.

### Step 3: (R)—N-(5-chloro-6-(1H-1,2,4-triazol-1-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide

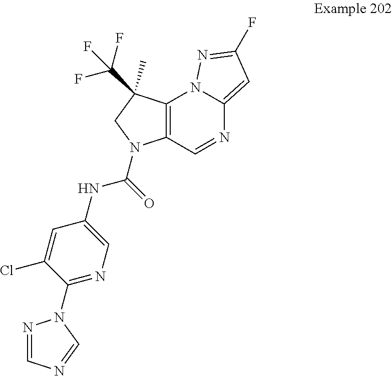 [SMILES: C[C@@]1(C(F)(F)F)CN(C(=O)Nc2cnc(-n3cncn3)c(Cl)c2)c2cnc3cc(F)nn3c21]

To a stirred mixture of 5-chloro-6-(1H-1,2,4-triazol-1-yl)pyridin-3-amine (18 mg, 92.4 μmol) in tetrahydrofuran (2 mL) was added triphosgene (13 mg, 46.1 μmol) and TEA (15 mg, 153.7 μmol) at 25° C. The resulting mixture was stirred at 25° C. for 1 h and then filtered. The filtrate was added to a solution of (R)-2-fluoro-8-methyl-8-(trifluoromethyl)-7,8-dihydro-6H-pyrazolo[1,5-a]pyrrolo[2,3-e]pyrimidine (Method K3 isomer 2; 220 mg, 76.8 μmol) in tetrahydrofuran (2 mL). To this solution were added TEA (77 mg, 768.6 μmol) and N,N-dimethylpyridin-4-amine (18 mg, 153.7 μmol). The reaction mixture was stirred at 40° C. for 2 h. The reaction mixture was concentrated under vacuum. The residue was purified by Prep-HPLC and the collected fractions were lyophilized to afford (R)—N-(5-chloro-6-(1H-1,2,4-triazol-1-yl)pyridin-3-yl)-2-fluoro-8-methyl-8-(trifluoromethyl)-7,8-dihydro-6H-pyrazolo[1,5-a]pyrrolo[2,3-e]pyrimidine-6-carboxamide (14 mg, 38% yield) as a white solid. The corresponding enantiomers of Example 202 can be prepared analogously using Method K3 isomer 1.

Example 202: ¹H NMR (300 MHz, DMSO-d₆) δ: 9.66 (br, 1H), 9.33 (s, 1H), 9.05 (s, 1H), 8.70 (s, 1H), 8.47 (d, J=2.1 Hz, 1H), 8.28 (s, 1H), 6.68 (d, J=5.7 Hz, 1H), 4.82 (d, J=11.4 Hz, 1H), 4.27 (d, J=11.4 Hz, 1H), 1.95 (s, 3H). LC-MS: m/z 482 [M+H]⁺.

### Method T6

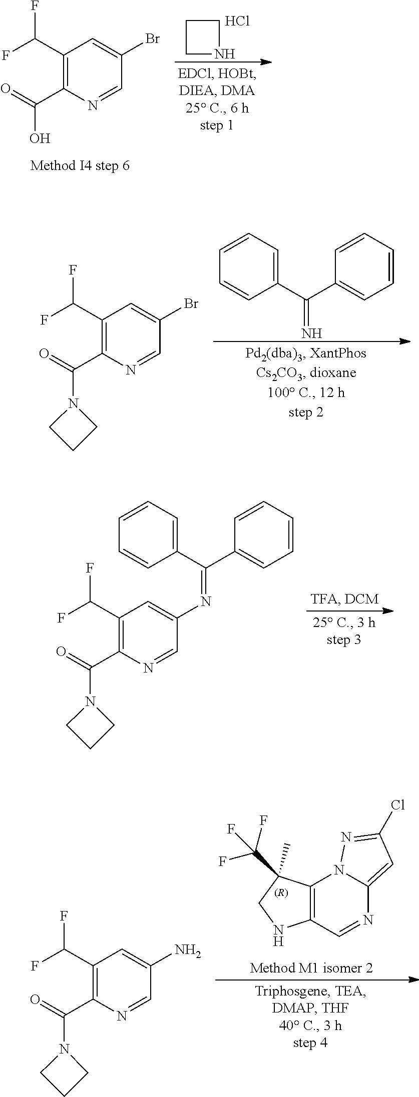

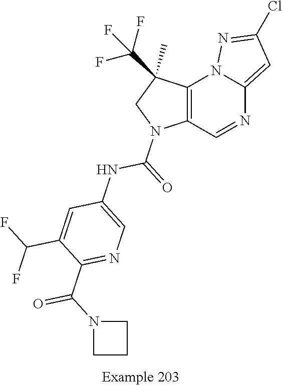 [SMILES: C[C@@]1(C(F)(F)F)CN(C(=O)Nc2cnc(C(=O)N3CCC3)c(C(F)F)c2)c2cnc3cc(Cl)nn3c21]

### Example 203: (R)—N-(6-(azetidine-1-carbonyl)-5-(difluoromethyl)pyridin-3-yl)-2-chloro-8-methyl-8-(trifluoromethyl)-7,8-dihydro-6H-pyrazolo[1,5-a]pyrrolo[2,3-e]pyrimidine-6-carboxamide

### Step 1: azetidin-1-yl(5-bromo-3-(difluoromethyl)pyridin-2-yl)methanone

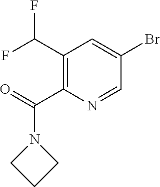 [SMILES: O=C(c1ncc(Br)cc1C(F)F)N1CCC1]

To a stirred solution of 5-bromo-3-(difluoromethyl)picolinic acid (Method 14 step 6; 300 mg, 1.2 mmol) in N,N-dimethylacetamide (2 mL) was added azetidine hydrogen chloride salt (145 mg, 1.6 mmol), EDCI (297 mg, 1.6 mmol), HOBt (209 mg, 1.6 mmol) and DIEA (615 mg, 4.8 mmol). The reaction mixture was stirred at 25° C. for 6 h. The reaction mixture was quenched with water (30 mL). The resulting solution was extracted with ethyl acetate (3×30 mL). The combined organic layers were washed with brine (50 mL), dried over anhydrous sodium sulfate and concentrated under vacuum. The residue was purified by column chromatography on silica gel using 50% petroleum ether and 50% ethyl acetate as eluent to afford azetidin-1-yl(5-bromo-3-(difluoromethyl)pyridin-2-yl)methanone (160 mg, 46% yield) as a yellow oil. LC-MS: m/z 291 [M+H]⁺.

### Step 2: azetidin-1-yl(3-(difluoromethyl)-5-((diphenylmethylene)amino)pyridin-2-yl)methanone

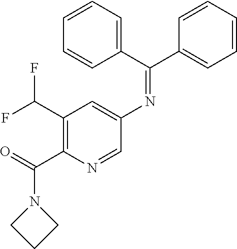 [SMILES: O=C(c1ncc(N=C(c2ccccc2)c2ccccc2)cc1C(F)F)N1CCC1]

To a mixture of azetidin-1-yl(5-bromo-3-(difluoromethyl)pyridin-2-yl)methanone (80 mg, 274.8 μmol) in dioxane (10 mL) was added diphenylmethanimine (100 mg, 549.6 μmol), Pd₂(dba)₃ (85 mg, 82.4 μmol), Xantphos (48 mg, 82.4 μmol) and Cs₂CO₃ (269 mg, 824.4 μmol) under nitrogen atmosphere. The resulting mixture was stirred at 100° C. for 12 h. The reaction mixture was concentrated under vacuum. The residue was diluted with water (10 mL), and the resulting solution was extracted with ethyl acetate (3×10 mL). The combined organic layers were dried over anhydrous sodium sulfate and concentrated under vacuum. The residue was purified by column chromatography on silica gel using 60% petroleum ether and 40% ethyl acetate to afford azetidin-1-yl(3-(difluoromethyl)-5-((diphenylmethylene)amino)pyridin-2-yl)methanone (80 mg, 74% yield) as a yellow solid. LC-MS: m/z 392 [M+H]⁺.

### Step 3: (5-amino-3-(difluoromethyl)pyridin-2-yl)(azetidin-1-yl)methanone

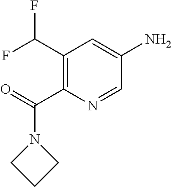 [SMILES: Nc1cnc(C(=O)N2CCC2)c(C(F)F)c1]

To a stirred mixture of azetidin-1-yl(3-(difluoromethyl)-5-((diphenylmethylene)amino)pyridin-2-yl)methanone (80 mg, 204.4 μmol) in dichloromethane (5 mL) were added TFA (1 mL). The resulting mixture was stirred at 25° C. for 3 h. The mixture was concentrated under vacuum. The residue was diluted with saturated aqueous NaHCO₃ solution (10 mL). The resulting solution was extracted with dichloromethane (3×10 mL). The combined organic layers were dried over anhydrous sodium sulfate and concentrated under vacuum. The residue was purified by column chromatography on silica gel using 90% dichloromethane and 10% methanol as eluent to afford (5-amino-3-(difluoromethyl)pyridin-2-yl)(azetidin-1-yl)methanone (40 mg, 86% yield) as a yellow solid. ¹H NMR (400 MHz, DMSO-d₆) δ 7.95 (d, J=2.4 Hz, 1H), 7.54 (t, J=56.0 Hz, 1H), 7.19 (d, J=2.4 Hz, 1H), 6.16 (br, 2H), 4.32-4.40 (m, 2H), 3.94-4.01 (m, 2H), 2.13-2.23 (m, 2H). LC-MS: m/z 228 [M+H]⁺.

### Step 4: (R)—N-(6-(azetidine-1-carbonyl)-5-(difluoromethyl)pyridin-3-yl)-2-chloro-8-methyl-8-(trifluoromethyl)-7,8-dihydro-6H-pyrazolo[1,5-a]pyrrolo[2,3-e]pyrimidine-6-carboxamide

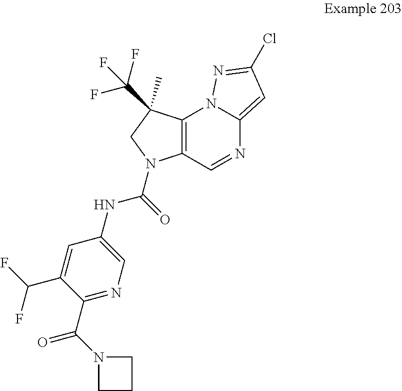 [SMILES: C[C@@]1(C(F)(F)F)CN(C(=O)Nc2cnc(C(=O)N3CCC3)c(C(F)F)c2)c2cnc3cc(Cl)nn3c21]

To a stirred solution of (5-amino-3-(difluoromethyl)pyridin-2-yl)(azetidin-1-yl)methanone (40 mg, 176.1 μmol) in tetrahydrofuran (2 mL) were added triphosgene (31 mg, 105.6 μmol) and TEA (27 mg, 264.1 μmol). The resulting mixture was stirred at 25° C. for 1 h and then filtered. The filtrate was added to a solution of Method M1 isomer 2 (58 mg, 211.3 μmol) in tetrahydrofuran (1 mL). To this solution were added N,N-dimethylpyridin-4-amine (43 mg, 352.1 μmol) and TEA (178 mg, 1.8 mmol). The mixture was stirred at 40° C. for 3 h. The mixture was concentrated under vacuum. The residue was purified by Prep-HPLC and the collected fractions were lyophilized to afford (R)—N-(6-(azetidine-1-carbonyl)-5-(difluoromethyl)pyridin-3-yl)-2-chloro-8-methyl-8-(trifluoromethyl)-7,8-dihydro-6H-pyrazolo[1,5-a]pyrrolo[2,3-e]pyrimidine-6-carboxamide (6 mg, 6% yield) as a white solid. The enantiomer of Example 203 can be prepared analogously using Method M1 isomer 1.

Example 203: ¹H NMR (400 MHz, DMSO-d₆) δ: 9.59 (s, 1H), 9.35 (s, 1H), 8.95 (d, J=2.4 Hz, 1H), 8.40 (d, J=2.4 Hz, 1H), 7.57 (t, J=56.0 Hz, 1H), 7.07 (s, 1H), 4.85 (d, J=11.6 Hz, 1H), 4.37-4.42 (m, 2H), 4.28 (d, J=11.6 Hz, 1H), 4.06-4.10 (m, 2H), 2.23-2.30 (m, 2H), 1.98 (s, 3H). LC-MS: m/z 530 [M+H]⁺.

### Method U6

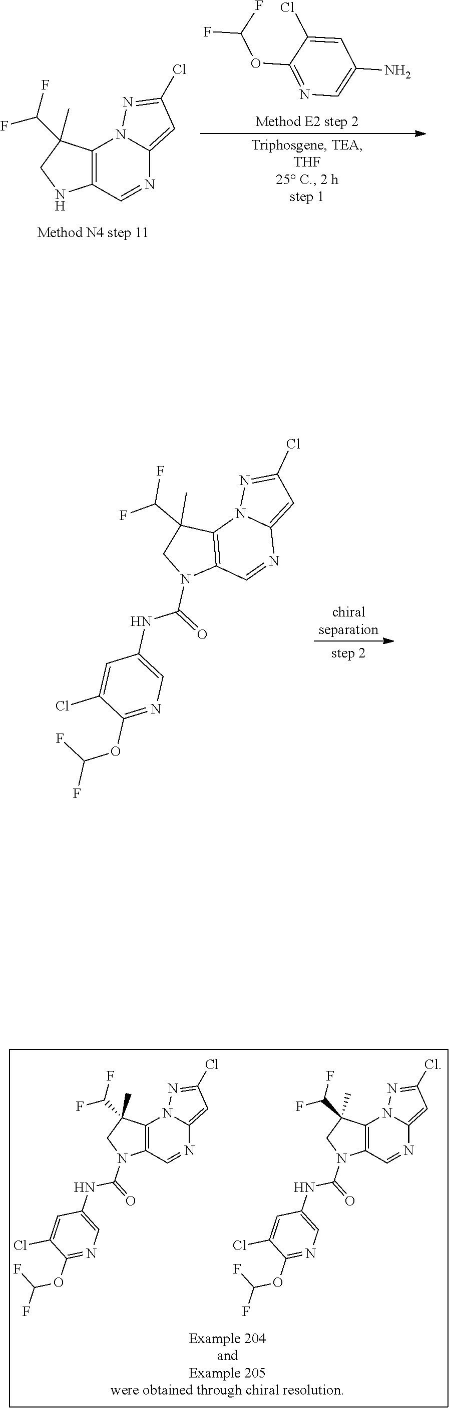 [SMILES: CC1(C(F)F)CNc2cnc3cc(Cl)nn3c21 ; Nc1cnc(OC(F)F)c(Cl)c1 ; CC1(C(F)F)CN(C(=O)Nc2cnc(OC(F)F)c(Cl)c2)c2cnc3cc(Cl)nn3c21 ; C[C@]1(C(F)F)CN(C(=O)Nc2cnc(OC(F)F)c(Cl)c2)c2cnc3cc(Cl)nn3c21 ; C[C@@]1(C(F)F)CN(C(=O)Nc2cnc(OC(F)F)c(Cl)c2)c2cnc3cc(Cl)nn3c21]

### Examples 204 and 205: (S)-2-chloro-N-(5-chloro-6-(difluoromethoxy)pyridin-3-yl)-8-(difluoromethyl)-8-methyl-7,8-dihydro-6H-pyrazolo[1,5-a]pyrrolo[2,3-e]pyrimidine-6-carboxamide and (R)-2-chloro-N-(5-chloro-6-(difluoromethoxy)pyridin-3-yl)-8-(difluoromethyl)-8-methyl-7,8-dihydro-6H-pyrazolo[1,5-a]pyrrolo[2,3-e]pyrimidine-6-carboxamide

### Step 1: 2-chloro-N-(5-chloro-6-(difluoromethoxy)pyridin-3-yl)-8-(difluoromethyl)-8-methyl-7,8-dihydro-6H-pyrazolo[1,5-a]pyrrolo[2,3-e]pyrimidine-6-carboxamide

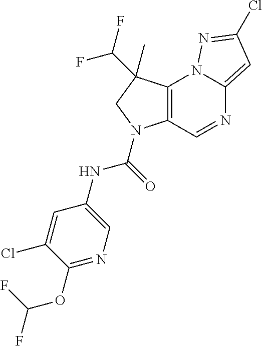 [SMILES: CC1(C(F)F)CN(C(=O)Nc2cnc(OC(F)F)c(Cl)c2)c2cnc3cc(Cl)nn3c21]

To a stirred solution of 5-chloro-6-(difluoromethoxy)pyridin-3-amine (Method E2 step 2; 137 mg, 698.6 μmol) in tetrahydrofuran (3 mL) were added triphosgene (82 mg, 278.3 μmol) and TEA (140 mg, 1.3 mmol). The resulting mixture was stirred at 25° C. for 0.5 h and then filtered. The filtrate was added to a solution of 2-chloro-8-(difluoromethyl)-8-methyl-7,8-dihydro-6H-pyrazolo[1,5-a]pyrrolo[2,3-e]pyrimidine (Method N4 step 11; 120 mg, 465.7 μmol) in tetrahydrofuran (1 mL). The mixture was stirred at 25° C. for 2 h. The mixture was concentrated under vacuum. The reaction mixture was quenched by the addition of water (50 mL). The resulting solution was extracted with ethyl acetate (3×50 mL). The combined organic layers were washed with brine (50 mL), dried over anhydrous sodium sulfate and concentrated under vacuum. The residue was purified by column chromatography on silica gel using 10% methanol and 90% dichloromethane as eluent to afford crude product. The crude product was purified by Prep-HPLC and the collected fractions were lyophilized to give 2-chloro-N-(5-chloro-6-(difluoromethoxy)pyridin-3-yl)-8-(difluoromethyl)-8-methyl-7,8-dihydro-6H-pyrazolo[1,5-a]pyrrolo[2,3-e]pyrimidine-6-carboxamide (60 mg, 26% yield) as an off-white solid. LC-MS: m/z 479 [M+H]⁺.

### Step 2: Separation of enantiomers to obtain (S)-2-chloro-N-(5-chloro-6-(difluoromethoxy)pyridin-3-yl)-8-(difluoromethyl)-8-methyl-7,8-dihydro-6H-pyrazolo[1,5-a]pyrrolo[2,3-e]pyrimidine-6-carboxamide and (R)-2-chloro-N-(5-chloro-6-(difluoromethoxy)pyridin-3-yl)-8-(difluoromethyl)-8-methyl-7,8-dihydro-6H-pyrazolo[1,5-a]pyrrolo[2,3-e]pyrimidine-6-carboxamide

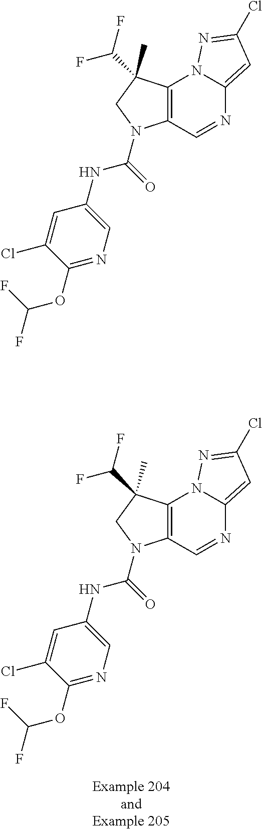 [SMILES: C[C@]1(C(F)F)CN(C(=O)Nc2cnc(OC(F)F)c(Cl)c2)c2cnc3cc(Cl)nn3c21 ; C[C@@]1(C(F)F)CN(C(=O)Nc2cnc(OC(F)F)c(Cl)c2)c2cnc3cc(Cl)nn3c21]

2-chloro-N-(5-chloro-6-(difluoromethoxy)pyridin-3-yl)-8-(difluoromethyl)-8-methyl-7,8-dihydro-6H-pyrazolo[1,5-a]pyrrolo[2,3-e]pyrimidine-6-carboxamide (50 mg, 104.3 μmol) was submitted to chiral-HPLC: Column: CHIRALPAK IA, 5×25 cm, 5 um; Mobile Phase A:Hex(0.5% 2M NH₃-MeOH)—HPLC, Mobile Phase B:EtOH-HPLC; Flow rate: 20 mL/min; Gradient: 10 B to 10 B in 18 min; 220/254 nm; RT1:15.075; RT2:23.483; Injection Volume: 0.5 ml; Number Of Runs: 16. The first eluting isomer was concentrated and lyophilized to afford Example 204 (14.2 mg, 28% yield) as an off-white solid and the second eluting isomer was concentrated and lyophilized to afford Example 205 (15.4 mg, 30% yield) as an off-white solid. Examples 204 and 205 are enantiomers, but their absolute stereochemistry is not yet known.

Example 204: ¹H NMR (400 MHz, DMSO-d₆) δ: 9.32 (s, 1H), 9.28 (br, 1H), 8.37 (d, J=2.4 Hz, 1H), 8.33 (d, J=2.4 Hz, 1H), 7.70 (t, J=72.0 Hz, 1H), 7.01 (s, 1H), 6.78 (t, J=56.0 Hz, 1H), 4.62 (d, J=10.8 Hz, 1H), 4.12 (d, J=10.8 Hz, 1H), 1.78 (s, 3H). LC-MS: m/z 479 [M+H]⁺.

Example 205: ¹H NMR (400 MHz, DMSO-d₆) δ: 9.32 (s, 1H), 9.28 (br, 1H), 8.37 (d, J=2.4 Hz, 1H), 8.33 (d, J=2.4 Hz, 1H), 7.70 (t, J=72.0 Hz, 1H), 7.01 (s, 1H), 6.78 (t, J=56.0 Hz, 1H), 4.62 (d, J=10.8 Hz, 1H), 4.12 (d, J=10.8 Hz, 1H), 1.78 (s, 3H). LC-MS: m/z 479 [M+H]⁺.

### Method V6

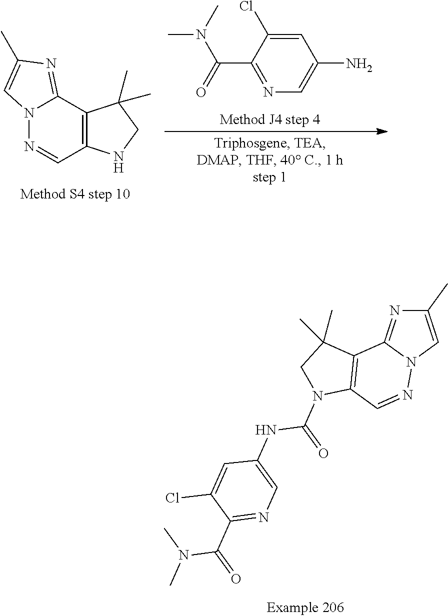

### Example 206: N-(5-chloro-6-(dimethylcarbamoyl)pyridin-3-yl)-2,9,9-timethyl-8,9-dihydro-7H-imidazo[1,2-b]pyrrolo[3,2-d]pyridazine-7-carboxamide

### Step 1: N-(5-chloro-6-(dimethylcarbamoyl)pyridin-3-yl)-2,9,9-trimethyl-8,9-dihydro-7H-imidazo[1,2-b]pyrrolo[3,2-d]pyridazine-7-carboxamide

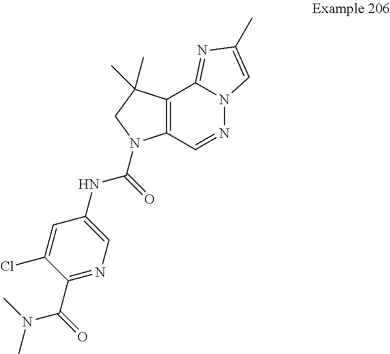

To a stirred solution of 5-amino-3-chloro-N,N-dimethylpicolinamide (Method J4 step 4; 7 mg, 34.6 μmol) in tetrahydrofuran (1 mL) were added triphosgene (6 mg, 20.7 μmol) and TEA (5 mg, 51.9 μmol). The resulting mixture was stirred at 25° C. for 0.5 h and then filtered. The filtrate was added to a solution of 2,9,9-trimethyl-8,9-dihydro-7H-imidazo[1,2-b]pyrrolo[3,2-d]pyridazine (Method S4 step 10; 7 mg, 34.6 μmol) in tetrahydrofuran (1 mL). To this solution was then added N,N-dimethylpyridin-4-amine (8 mg, 69.2 μmol) and TEA (35 mg, 346.1 μmol). The reaction mixture was stirred at 40° C. for 1 h. The reaction mixture was concentrated under vacuum. The residue was purified by Prep-HPLC and the collected fractions were lyophilized to afford N-(5-chloro-6-(dimethylcarbamoyl)pyridin-3-yl)-2,9,9-trimethyl-8,9-dihydro-7H-imidazo[1,2-b]pyrrolo[3,2-d]pyridazine-7-carboxamide (7 mg, 51% yield) as a white solid.

Example 206: ¹H NMR (400 MHz, DMSO-d₆) δ: 9.24 (br, 1H), 9.13 (s, 1H), 8.74 (d, J=2.0 Hz, 1H), 8.29 (d, J=2.0 Hz, 1H), 7.97 (s, 1H), 4.08 (s, 2H), 3.02 (s, 3H), 2.78 (s, 3H), 2.37 (s, 3H), 1.60 (s, 6H). LC-MS: m/z 428 [M+H]⁺.

### Method W6

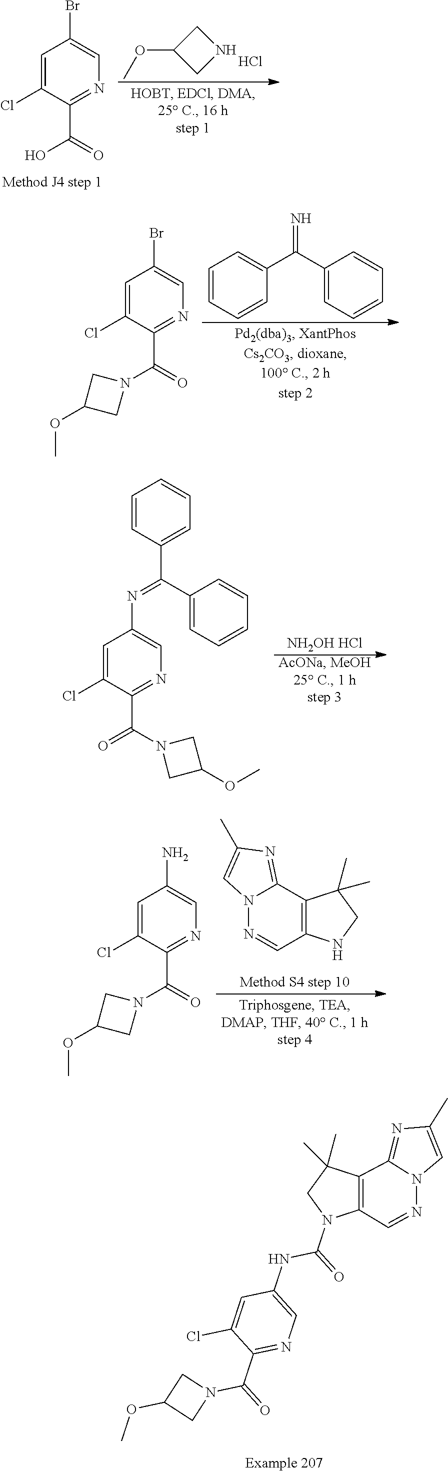

### Example 207: N-(5-chloro-6-(3-methoxyazetidine-1-carbonyl)pyridin-3-yl)-2,9,9-trimethyl-8,9-dihydro-7H-imidazo[1,2-b]pyrrolo[3,2-d]pyridazine-7-carboxamide

### Step 1: (5-bromo-3-chloropyridin-2-yl)(3-methoxyazetidin-1-yl)methanone

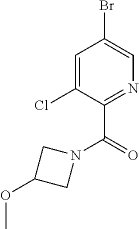 [SMILES: COC1CN(C(=O)c2ncc(Br)cc2Cl)C1]

To a stirred solution of 5-bromo-3-chloropicolinic acid (Method J4 step 1; 900 mg, 3.8 mmol) in N,N-dimethylacetamide (25 mL) was added 3-methoxyazetidine hydrochloride (949 mg, 4.9 mmol), HOBt (669 mg, 4.9 mmol), EDCI (950 mg, 4.9 mmol) and DIEA (1.5 g, 11.4 mmol). The reaction mixture was stirred at 25° C. for 16 h. The reaction mixture was quenched with water (200 mL). The resulting solution was extracted with ethyl acetate (3×200 mL). The combined organic layers were washed with brine (3×500 mL), dried over anhydrous sodium sulfate and concentrated under vacuum. The residue was purified by column chromatography on silica gel using 97% dichloromethane and 3% methanol as eluent to afford (5-bromo-3-chloropyridin-2-yl)(3-methoxyazetidin-1-yl)methanone (900 mg, 73% yield) as a colorless oil. ¹H NMR (400 MHz, DMSO-d₆) δ: 8.72 (d, J=2.0 Hz, 1H), 8.50 (d, J=2.0 Hz, 1H), 4.23-4.29 (m, 2H), 4.12-4.17 (m, 1H), 3.77-3.93 (m, 2H), 3.20 (s, 3H). LC-MS: m/z 305 [M+H]⁺.

### Step: 2: (3-chloro-5-((diphenylmethylene)amino)pyridin-2-yl)(3-methoxyazetidin-1-yl)methanone

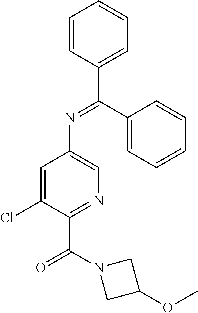 [SMILES: COC1CN(C(=O)c2ncc(N=C(c3ccccc3)c3ccccc3)cc2Cl)C1]

To a stirred solution of (5-bromo-3-chloropyridin-2-yl)(3-methoxyazetidin-1-yl)methanone (600 mg, 2.0 mmol) in dioxane (18 mL) was added diphenylmethanimine (534 mg, 2.9 mmol), Pd₂(dba)₃ (203 mg, 196.1 μmol), Xantphos (114 mg, 196.4 μmol) and Cs₂CO₃ (1.9 g, 5.9 mmol) under nitrogen atmosphere. The reaction mixture was stirred at 100° C. for 2 h. The reaction mixture was cooled to 25° C. The resulting mixture was concentrated under vacuum. The residue was diluted with water (50 mL) and extracted with ethyl acetate (3×50 mL). The combined organic layers were dried over anhydrous sodium sulfate and concentrated under vacuum. The residue was purified by column chromatography using 98% dichloromethane and 2% methanol as eluent to afford (3-chloro-5-((diphenylmethylene)amino)pyridin-2-yl)(3-methoxyazetidin-1-yl)methanone (500 mg, 56% yield) as a brown oil. LC-MS: m/z 406 [M+H]⁺.

### Step 3: (5-amino-3-chloropyridin-2-yl)(3-methoxyazetidin-1-yl)methanone

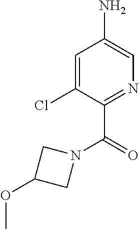 [SMILES: COC1CN(C(=O)c2ncc(N)cc2Cl)C1]

To a stirred solution of (3-chloro-5-((diphenylmethylene)amino)pyridin-2-yl)(3-methoxyazetidin-1-yl)methanone (400 mg, 985.5 μmol) in methanol (12 mL) were added hydroxylamine hydrochloride (171 mg, 2.5 mmol) and sodium acetate (242 mg, 2.9 mmol). The reaction mixture was stirred at 25° C. for 1 h. The resulting mixture was concentrated under vacuum. The residue was purified by column chromatography on silica gel using 95% dichloromethane and 5% methanol as eluent to afford (5-amino-3-chloropyridin-2-yl)(3-methoxyazetidin-1-yl)methanone (200 mg, 75% yield) as a white solid. LC-MS: m/z 242 [M+H]⁺.

### Step 4: N-(5-chloro-6-(3-methoxyazetidine-1-carbonyl)pyridin-3-yl)-2,9,9-trimethyl-8,9-dihydro-7H-imidazo[1,2-b]pyrrolo[3,2-d]pyridazine-7-carboxamide

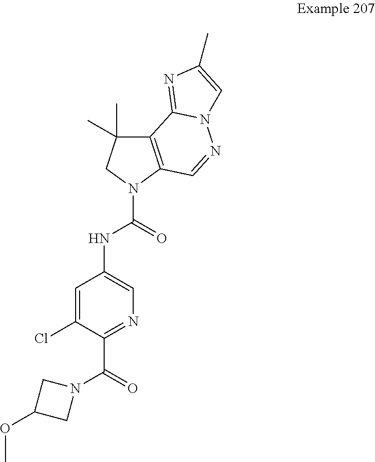 [SMILES: COC1CN(C(=O)c2ncc(NC(=O)N3CC(C)(C)c4c3cnn3cc(C)nc43)cc2Cl)C1]

To a stirred solution of (5-amino-3-chloropyridin-2-yl)(3-methoxyazetidin-1-yl)methanone (11 mg, 44.5 μmol) in tetrahydrofuran (1 mL) were added triphosgene (7 mg, 22.2 μmol) and TEA (6 mg, 55.6 μmol). The resulting mixture was stirred at 25° C. for 0.5 h and then filtered. The filtrate was added to a solution of 2,9,9-trimethyl-8,9-dihydro-7H-imidazo[1,2-b]pyrrolo[3,2-d]pyridazine (Method S4 step 10; 7 mg, 37.1 μmol) in tetrahydrofuran (1 mL). To this solution were added N,N-dimethylpyridin-4-amine (9 mg, 74.2 μmol) and TEA (38 mg, 370.8 μmol). The mixture was stirred at 40° C. for 1 h. The mixture was concentrated under vacuum. The residue was purified by Prep-HPLC and the collected fractions were lyophilized to afford N-(5-chloro-6-(3-methoxyazetidine-1-carbonyl)pyridin-3-yl)-2,9,9-trimethyl-8,9-dihydro-7H-imidazo[1,2-b]pyrrolo[3,2-d]pyridazine-7-carboxamide (6 mg, 36% yield) as a white solid.

Example 207: ¹H NMR (400 MHz, DMSO-d₆) δ: 9.29 (br, 1H), 9.12 (s, 1H), 8.74 (d, J=2.0 Hz, 1H), 8.29 (d, J=2.0 Hz, 1H), 7.97 (s, 1H), 4.18-4.27 (m, 3H), 4.08 (s, 2H), 3.84-3.89 (m, 2H), 3.21 (s, 3H), 2.37 (s, 3H), 1.60 (s, 6H). LC-MS: m/z 470 [M+H]⁺.

### Method X6

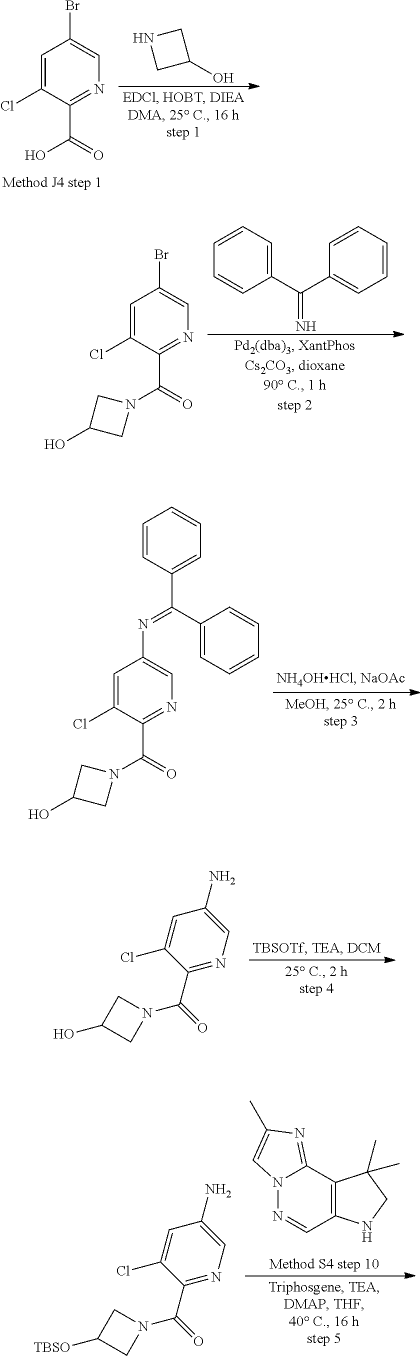

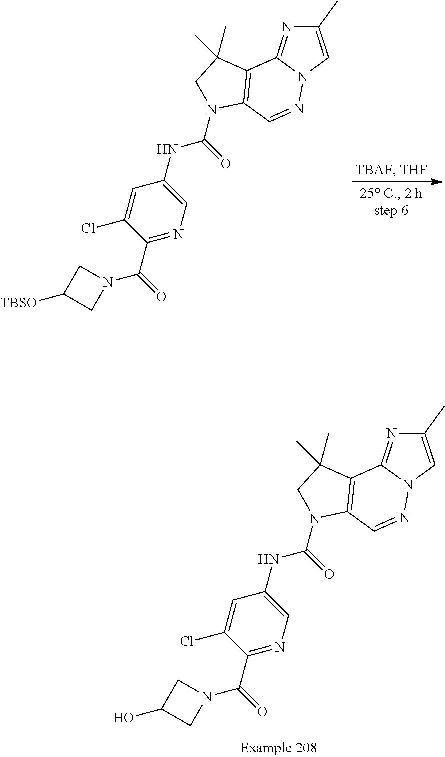 [SMILES: Cc1cn2ncc3c(c2n1)C(C)(C)CN3C(=O)Nc1cnc(C(=O)N2CC(O[Si](C)(C)C(C)(C)C)C2)c(Cl)c1 ; Cc1cn2ncc3c(c2n1)C(C)(C)CN3C(=O)Nc1cnc(C(=O)N2CC(O)C2)c(Cl)c1]

### Examples 208: N-(5-chloro-6-(3-hydroxyazetidine-1-carbonyl)pyridin-3-yl)-2,9,9-trimethyl-8,9-dihydro-7H-imidazo[1,2-b]pyrrolo[3,2-d]pyridazine-7-carboxamide

### Step 1: (5-bromo-3-chloropyridin-2-yl)(3-hydroxyazetidin-1-yl)methanone

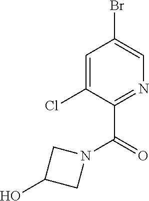 [SMILES: O=C(c1ncc(Br)cc1Cl)N1CC(O)C1]

To a stirred solution of 5-bromo-3-chloropicolinic acid (Method J4 step 1; 1.80 g, 7.6 mmol) in N,N-dimethylacetamide (10 mL) were added azetidin-3-ol (834 mg, 7.6 mmol), EDCI (1.90 g, 9.9 mmol), DIEA (2.95 g, 22.8 mmol) and HOBT (1.34 g, 9.9 mmol). The mixture was stirred at 25° C. for 16 h. The reaction mixture was quenched with water (100 mL). The resulting mixture was extracted with ethyl acetate (3×100 mL). The combined organic layers were washed with brine (100 mL), dried over anhydrous sodium sulfate and concentrated under vacuum. The residue was purified by column chromatography using 50% petroleum ether and 50% ethyl acetate as eluent to afford (5-bromo-3-chloropyridin-2-yl)(3-hydroxyazetidin-1-yl)methanone (800 mg, 36% yield) as yellow oil. LC-MS: m/z 291 [M+H]⁺.

### Step 2: (3-chloro-5-((diphenylmethylene)amino)pyridin-2-yl)(3-hydroxyazetidin-1-yl)methanone

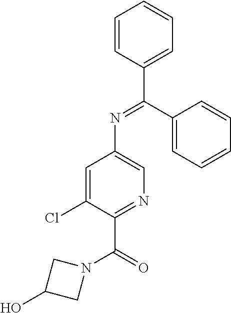 [SMILES: O=C(c1ncc(N=C(c2ccccc2)c2ccccc2)cc1Cl)N1CC(O)C1]

To a stirred solution of (5-bromo-3-chloropyridin-2-yl)(3-hydroxyazetidin-1-yl)methanone (300 mg, 1.0 mmol) in dioxane (20 mL) were added diphenylmethanimine (223 mg, 1.2 mmol), XantPhos (178 mg, 308 μmol), Pd₂(dba)₃ (188 mg, 205 μmol) and Cs₂CO₃ (1.01 g, 3.1 mmol). The mixture was stirred at 90° C. for 1 h under nitrogen atmosphere. After cooled to 25° C., the solid was filtered out. The filtrate was concentrated under vacuum. The residue was purified by Prep-TLC using 90% petroleum ether and 10% ethyl acetate as eluent to afford (3-chloro-5-((diphenylmethylene)amino)pyridin-2-yl)(3-hydroxyazetidin-1-yl)methanone (180 mg, 44% yield) as yellow solid. LC-MS: m/z 392 [M+H]⁺.

### Step 3: (5-amino-3-chloropyridin-2-yl)(3-hydroxyazetidin-1-yl)methanone

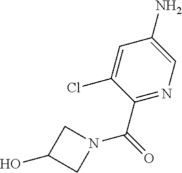 [SMILES: Nc1cnc(C(=O)N2CC(O)C2)c(Cl)c1]

To a stirred solution of (3-chloro-5-((diphenylmethylene)amino)pyridin-2-yl)(3-hydroxyazetidin-1-yl)methanone (180 mg, 459 μmol) in methanol (5 mL) were added hydroxylamine hydrochloride (80 mg, 1.1 mmol) and sodium acetate (113 mg, 1.4 mmol). The mixture was stirred at 25° C. for 2 h. The resulting mixture was concentrated under vacuum. The residue was submitted to Prep-HPLC purification and the collected fractions were concentrated to give (5-amino-3-chloropyridin-2-yl)(3-hydroxyazetidin-1-yl)methanone (90 mg, 86% yield) as yellow solid. LC-MS: m/z 228 [M+H]⁺.

### Step 4: (5-amino-3-chloropyridin-2-yl)(3-((tert-butyldimethylsilyl)oxy)azetidin-1-yl)methanone

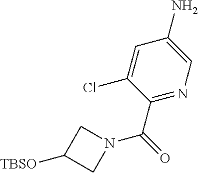 [SMILES: CC(C)(C)[Si](C)(C)OC1CN(C(=O)c2ncc(N)cc2Cl)C1]

To a stirred solution of (5-amino-3-chloropyridin-2-yl)(3-hydroxyazetidin-1-yl)methanone (80 mg, 351 μmol) in dichloromethane (2 mL) were added trifluoromethanesulfonic acid tert-butyldimethylsilyl ester (557 mg, 2.1 mmol) and TEA (213 mg, 2.1 mmol) at 0° C. The mixture was stirred at 25° C. for 2 h. The resulting mixture was concentrated under vacuum. The residue was purified by column chromatography using 50% petroleum ether and 50% ethyl acetate as eluent to afford (5-amino-3-chloropyridin-2-yl)(3-((tert-butyldimethylsilyl)oxy)azetidin-1-yl)methanone (95 mg, 79% yield) as a white solid. ¹H NMR (300 MHz, Methanol-d₄) δ: 7.88 (d, J=2.4 Hz, 1H), 7.08 (d, J=2.4 Hz, 1H), 4.72-4.81 (m, 1H), 4.28-4.43 (m, 2H), 3.88-4.05 (m, 2H), 0.93 (s, 9H), 0.02 (s, 6H). LC-MS: m/z 342 [M+H]⁺.

### Step 5: N-(6-(3-((tert-butyldimethylsilyl)oxy)azetidine-1-carbonyl)-5-chloropyridin-3-yl)-2,9,9-trimethyl-8,9-dihydro-7H-imidazo[1,2-b]pyrrolo[3,2-d]pyridazine-7-carboxamide

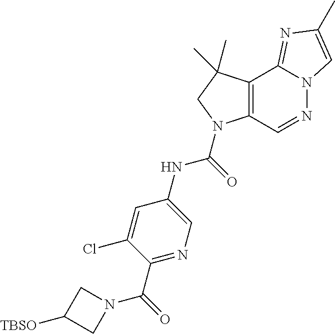 [SMILES: Cc1cn2ncc3c(c2n1)C(C)(C)CN3C(=O)Nc1cnc(C(=O)N2CC(O[Si](C)(C)C(C)(C)C)C2)c(Cl)c1]

To a stirred solution of (5-amino-3-chloropyridin-2-yl)(3-((tert-butyldimethylsilyl)oxy)azetidin-1-yl)methanone (50 mg, 148.3 μmol) in tetrahydrofuran (5 mL) were added triphosgene (17 mg, 57.3 μmol) and TEA (15 mg, 148.33 μmol). The resulting mixture was stirred at 25° C. for 0.5 h and then filtered. The resulting filtrate was added to a solution of 2,9,9-trimethyl-8,9-dihydro-7H-imidazo[1,2-b]pyrrolo[3,2-d]pyridazine (Method S4 step 10; 20 mg, 98 μmol) in tetrahydrofuran (2 mL). To this solution was then added TEA (100 mg, 988 μmol) and N,N-dimethylpyridin-4-amine (24 mg, 197 μmol). The mixture was stirred at 40° C. for 16 h. The mixture was quenched with water (10 mL), and the resulting solution was extracted with ethyl acetate (3×10 mL). The combined organic layers were washed with brine (20 mL), dried over anhydrous sodium sulfate and concentrated under vacuum. The residue was purified by Prep-TLC using 50% petroleum ether and 50% ethyl acetate as eluent to afford N-(6-(3-((tert-butyldimethylsilyl)oxy)azetidine-1-carbonyl)-5-chloropyridin-3-yl)-2,9,9-trimethyl-8,9-dihydro-7H-imidazo[1,2-b]pyrrolo[3,2-d]pyridazine-7-carboxamide (20 mg, 35% yield) as a yellow solid. LC-MS: m/z 570 [M+H]⁺.

### Step 6: N-(5-chloro-6-(3-hydroxyazetidine-1-carbonyl)pyridin-3-yl)-2,9,9-trimethyl-8,9-dihydro-7H-imidazo[1,2-b]pyrrolo[3,2-d]pyridazine-7-carboxamide

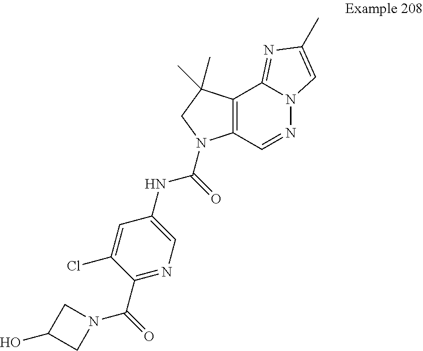 [SMILES: Cc1cn2ncc3c(c2n1)C(C)(C)CN3C(=O)Nc1cnc(C(=O)N2CC(O)C2)c(Cl)c1]

To a solution of N-(6-(3-((tert-butyldimethylsilyl)oxy)azetidine-1-carbonyl)-5-chloropyridin-3-yl)-2,9,9-trimethyl-8,9-dihydro-7H-imidazo[1,2-b]pyrrolo[3,2-d]pyridazine-7-carboxamide (18 mg, 31.57 μmol) in tetrahydrofuran (2 mL) was tetrabutylammonium fluoride (1 mL, 1 M in tetrahydrofuran). The mixture was stirred at 25° C. for 2 h. The resulting mixture was concentrated under vacuum. The residue was purified by Prep-TLC using 90% dichloromethane ether and 10% methanol as eluent to afford a crude product (30 mg). The crude product was submitted to Prep-HPLC purification and the collected fractions were lyophilized to give N-(5-chloro-6-(3-hydroxyazetidine-1-carbonyl)pyridin-3-yl)-2,9,9-trimethyl-8,9-dihydro-7H-imidazo[1,2-b]pyrrolo[3,2-d]pyridazine-7-carboxamide (3.7 mg, 25% yield) as a white solid.

Example 208: ¹H NMR (400 MHz, DMSO-d₆) δ: 9.28 (br, 1H), 9.12 (s, 1H), 8.74 (d, J=2.4 Hz, 1H), 8.29 (d, J=2.4 Hz, 1H), 7.98 (s, 1H), 5.82 (s, 1H), 4.51 (s, 1H), 4.20-4.29 (m, 1H), 4.12-4.19 (m, 1H), 4.08 (s, 2H), 3.74-3.83 (m, 2H), 2.37 (s, 3H), 1.60 (s, 6H). LC-MS: m/z 456 [M+H]⁺.

### Method Y6

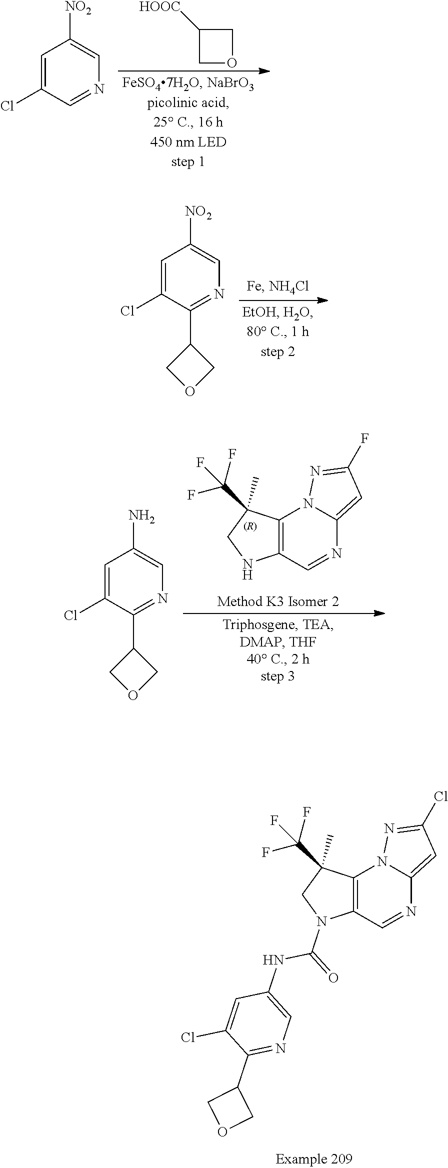

### Example 209: (R)-2-chloro-N-(5-chloro-6-(oxetan-3-yl)pyridin-3-yl)-8-methyl-8 (trifluoromethyl)-7,8-dihydro-6H-pyrazolo[1,5-a]pyrrolo[2,3-e]pyrimidine-6-carboxamide

### Step 1: 3-chloro-5-nitro-2-(oxetan-3-yl)pyridine

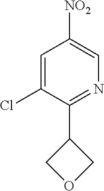 [SMILES: O=[N+]([O-])c1cnc(C2COC2)c(Cl)c1]

To a stirred mixture of 3-chloro-5-nitropyridine (158 mg, 1.0 mmol) in dimethyl sulfoxide (2 mL) were added oxetane-3-carboxylic acid (1.0 g, 10.0 mmol), picolinic acid (13 mg, 0.1 mmol), sodium bromate (300 mg, 2.0 mmol) and ferrous sulfate heptahydrate (14 mg, 50 μmol). The reaction mixture was stirred at 25° C. for 16 h under nitrogen irradiated with 450 nm LED. The residue was diluted with sodium hydroxide aqueous solution (50 mL, 1 M), and the resulting solution was extracted with ethyl acetate (3×50 mL). The combined organic layers were dried over anhydrous sodium sulfate and concentrated under vacuum. The residue was purified by column chromatography on silica gel using 50% petroleum ether and 50% ethyl acetate as eluent to afford 3-chloro-5-nitro-2-(oxetan-3-yl)pyridine (17 mg, 8% yield) as a yellow oil. ¹H NMR (300 MHz, Chloroform-d) δ: 9.39 (d, J=2.4 Hz, 1H), 8.49 (d, J=2.4 Hz, 1H), 4.95-5.15 (m, 4H), 4.67-4.91 (m, 1H). LC-MS: m/z 215 [M+H]⁺.

### Step 2: 5-chloro-6-(oxetan-3-yl)pyridin-3-amine

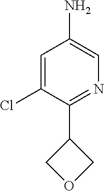 [SMILES: Nc1cnc(C2COC2)c(Cl)c1]

To a solution of 3-chloro-5-nitro-2-(oxetan-3-yl)pyridine (70 mg, 326 μmol) in ethanol (12 mL) and water (4 mL) were added Fe (91 mg, 1.6 mmol), NH₄Cl (52 mg, 978 μmol). The resulting mixture was stirred at 80° C. for 1 h. The mixture was cooled down to 25° C. The reaction mixture was filtered, and the collected solid was washed with ethyl acetate (3×25 mL). The combined organic layers were dried over anhydrous sodium sulfate and concentrated under vacuum. The residue was purified by column chromatography on silica gel using 25% petroleum ether and 75% ethyl acetate as eluent to afford 5-chloro-6-(oxetan-3-yl)pyridin-3-amine (30 mg, 49% yield) as a yellow oil. ¹H NMR (300 MHz, DMSO-d₆) δ: 7.91 (d, J=3.0 Hz, 1H), 6.98 (d, J=3.0 Hz, 1H), 5.54 (br, 2H), 4.71-4.83 (m, 4H), 4.384.54 (m, 1H). LC-MS: m/z 185 [M+H]⁺.

### Step 3: (R)-2-chloro-N-(5-chloro-6-(oxetan-3-yl)pyridin-3-yl)-8-methyl-8-(trifluoromethyl)-7,8-dihydro-6H-pyrazolo[1,5-a]pyrrolo[2,3-e]pyrimidine-6-carboxamide

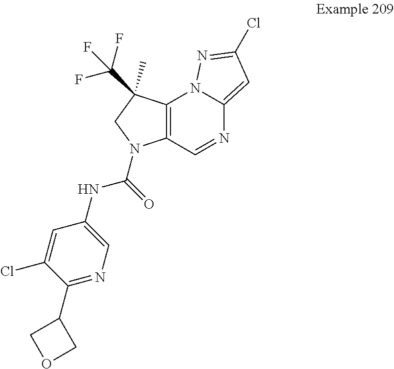 [SMILES: C[C@@]1(C(F)(F)F)CN(C(=O)Nc2cnc(C3COC3)c(Cl)c2)c2cnc3cc(Cl)nn3c21]

To a stirred solution of 5-chloro-6-(oxetan-3-yl)pyridin-3-amine (32 mg, 173 μmol) in tetrahydrofuran (8 mL) was added triphosgene (26 mg, 87 μmol) and TEA (26 mg, 260 μmol). The resulting mixture was stirred at 40° C. for 0.5 h and then filtered. The resulting filtrate was added to a solution of Method M1 isomer 2 (48 mg, 173 μmol) in tetrahydrofuran (1.5 mL). To this solution was then added TEA (175 mg, 1.7 mmol) and N,N-dimethylpyridin-4-amine (42 mg, 346 μmol). The mixture was stirred at 40° C. for 2 h. The solvent was concentrated under vacuum. The residue was purified by Prep-TLC using 97% dichloromethane and 3% methanol as eluent to afford 20 mg of crude product. The afforded crude product was purified by Prep-HPLC purification and the collected fractions were lyophilized to give (R)-2-chloro-N-(5-chloro-6-(oxetan-3-yl)pyridin-3-yl)-8-methyl-8-(trifluoromethyl)-7,8-dihydro-6H-pyrazolo[1,5-a]pyrrolo[2,3-e]pyrimidine-6-carboxamide (6 mg, 7% yield) as an off-white solid. The enantiomer of Example 209 can be prepared analogously using Method M1 isomer 1.

Example 209: ¹H NMR (400 MHz, DMSO-d₆) δ: 9.41 (br, 1H), 9.34 (s, 1H), 8.73 (d, J=2.0 Hz, 1H), 8.19 (d, J=2.0 Hz, 1H), 7.06 (s, 1H), 4.80-4.95 (m, 5H), 4.60-4.69 (m, 1H), 4.28 (d, J=12.0 Hz, 1H), 1.98 (s, 3H). LC-MS: m/z 487 [M+H]⁺.

### Method Z6

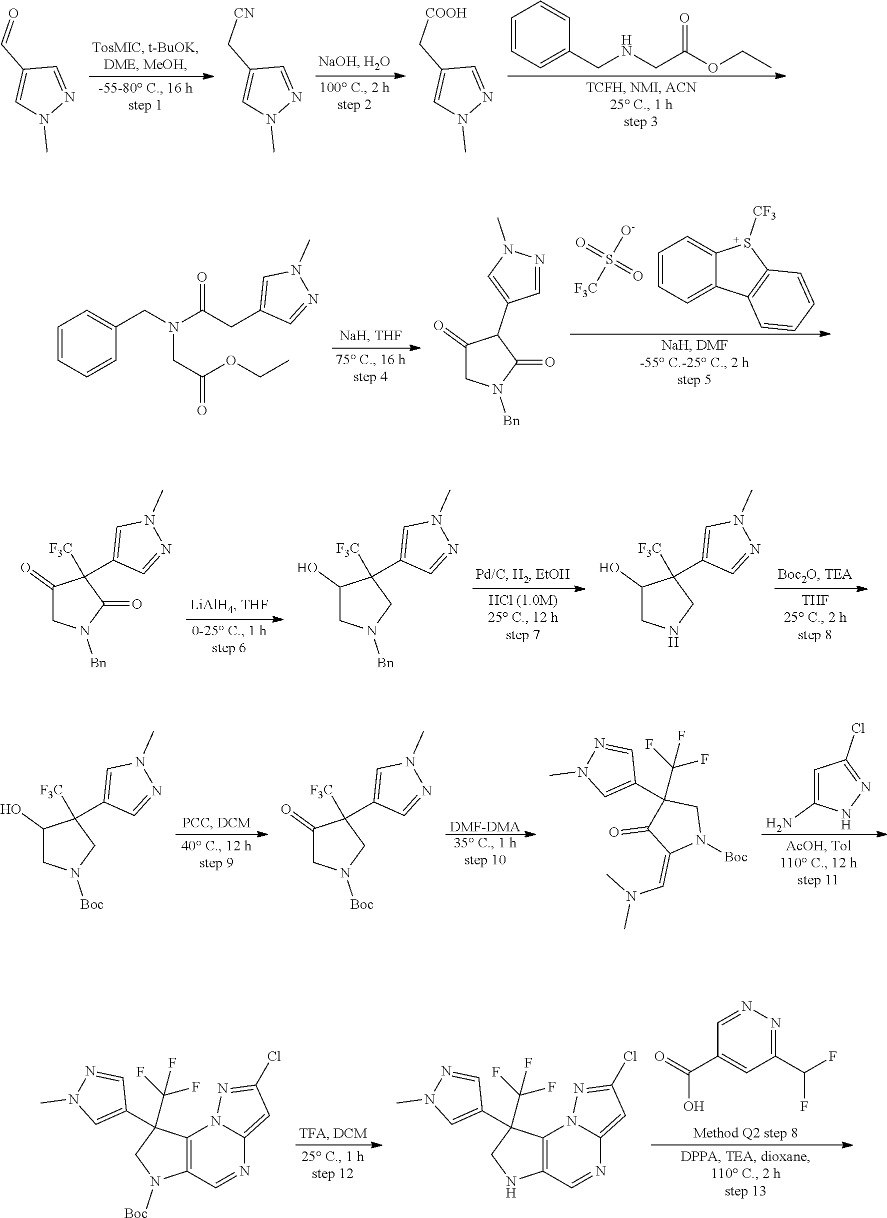

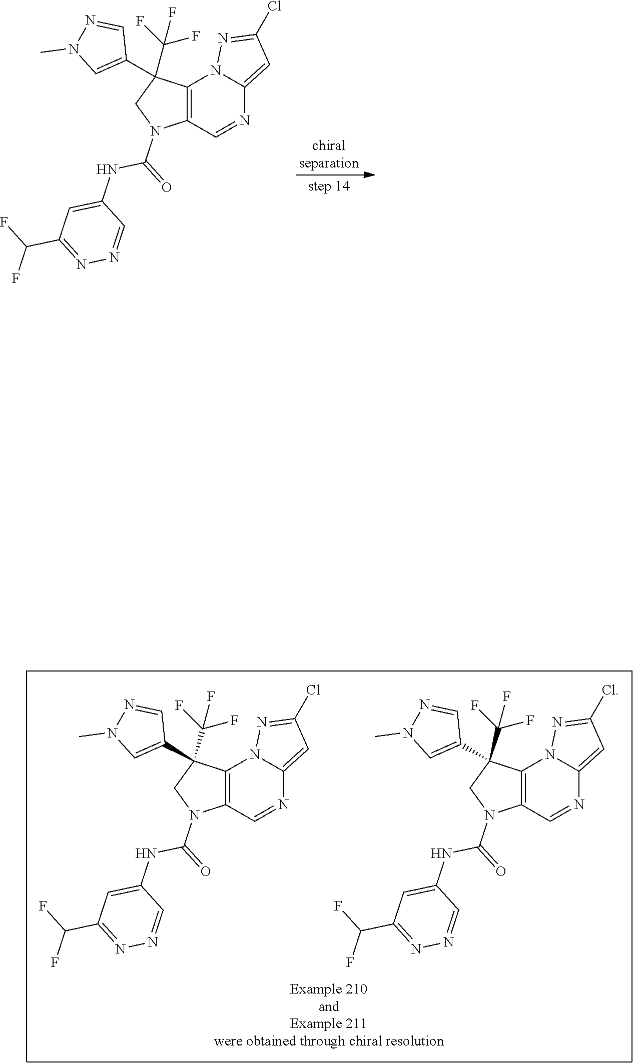 [SMILES: Cn1cc(C2(C(F)(F)F)CN(C(=O)Nc3cnnc(C(F)F)c3)c3cnc4cc(Cl)nn4c32)cn1 ; Cn1cc([C@]2(C(F)(F)F)CN(C(=O)Nc3cnnc(C(F)F)c3)c3cnc4cc(Cl)nn4c32)cn1 ; Cn1cc([C@@]2(C(F)(F)F)CN(C(=O)Nc3cnnc(C(F)F)c3)c3cnc4cc(Cl)nn4c32)cn1]

### Examples 210 and 211: Single enantiomers obtained from a racemic mixture containing (S)-2-chloro-N-(6-(difluoromethyl)pyridazin-4-yl)-8-(1-methyl-1H-pyrazol-4-yl)-8-(trifluoromethyl)-7,8-dihydro-6H-pyrazolo[1,5-a]pyrrolo[2,3-e]pyrimidine-&-carboxamide and (R)?chloro-N-(6-(difluoromethyl)pyridazin-4-yl-8(1-methyl-H-pyrazol-4-yl)-8-(trifluoromethyl)-7,8-dihydro-6H-pyrazolo[1,5-a]pyrrolo[2,3-e]pyrimidine-&-carboxamide

### Step 1: 2-(1-methyl-1H-pyrazol-4-yl)acetonitrile

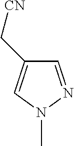

To a solution of potassium tert-butoxide (203.81 g, 1.8 mol) in 1,2-dimethoxy-ethan (600 mL) was added sosylmethyl isocyanide (186.17 g, 953.5 mmol) in 1,2-dimethoxy-ethan (800 mL) and 1-methylpyrazole-4-carbaldehyde (100 g, 908.1 mmol) in 1,2-dimethoxy-ethan (600 mL). The reaction mixture was stirred at −55° C. for 1 h. Then methanol (1000 mL) was added into the mixture. The resulting solution was stirred at 80° C. for 16 h. After cooled to 25° C., the reaction mixture was concentrated and then quenched with water (1000 mL). The resulting solution was extracted with ethyl acetate (3×1000 mL). The combined organic layers were dried over anhydrous sodium sulfate and concentrated under vacuum to afford 2-(1-methyl-1H-pyrazol-4-yl)acetonitrile (60 g, 54% yield) as a yellow oil. ¹H NMR (300 MHz, Chloroform-d) δ: 7.40 (s, 1H), 7.37 (s, 1H), 3.86 (s, 3H), 3.56 (s, 2H). LC-MS: m/z 122 [M+H]⁺.

### Step 2: 2-(1-methyl-1H-pyrazol-4-yl)acetic acid

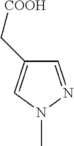

To a solution of 2-(1-methyl-1H-pyrazol-4-yl)acetonitrile (70 g, 577.8 mmol) in water (200 mL) was added sodium hydroxide (115.56 g, 2.9 mol) in water (200 mL). The resulting solution was stirred at 100° C. for 2 h. After cooled to 25° C., the reaction mixture was washed with ethyl acetate (2×300 mL). The pH of the aqueous layer was adjusted to 3-4 with HCl (1 M). The resulting solution was extracted with ethyl acetate (6×500 mL). The combined organic layers were dried over anhydrous sodium sulfate and concentrated under vacuum to afford 2-(1-methyl-1H-pyrazol-4-yl)acetic acid (55 g, 68% yield) as a yellow solid. ¹H NMR (300 MHz, DMSO-d₆) δ: 12.25 (br, 1H), 7.55 (s, 1H), 7.29 (s, 1H), 3.78 (s, 3H), 3.39 (s, 2H). LC-MS: m/z 141 [M+H]⁺.

### Step 3: ethyl N-benzyl-N-(2-(1-methyl-1H-pyrazol-4-yl)acetyl)glycinate

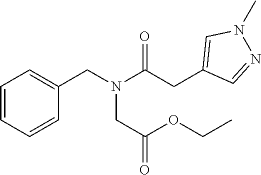 [SMILES: CCOC(=O)CN(Cc1ccccc1)C(=O)Cc1cnn(C)c1]

To a solution of 2-(1-methyl-1H-pyrazol-4-yl)acetic acid (50 g, 356.8 mmol) in acetonitrile (1000 mL) was added ethyl benzylglycinate (68.95 g, 356.8 mmol), TCFH (150.16 g, 535.2 mmol) and NMI (87.88 g, 1.0 mol). The resulting solution was stirred at 25° C. for 1 h. The reaction mixture was concentrated and then quenched with water (1000 mL). The resulting solution was extracted with ethyl acetate (3×1000 mL). The combined organic layers were dried over anhydrous sodium sulfate and concentrated under vacuum. The residue was purified by prep-HPLC and the collected fractions were concentrated to afford ethyl N-benzyl-N-(2-(1-methyl-1H-pyrazol-4-yl)acetyl)glycinate (100 g, 88% yield) as a yellow oil. LC-MS: m/z 316 [M+H]⁺.

### Step 4: 1-benzyl-3-(1-methyl-1H-pyrazol-4-yl)pyrrolidine-2,4-dione

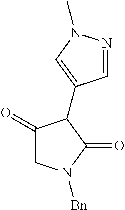 [SMILES: Cn1cc(C2C(=O)CN(Cc3ccccc3)C2=O)cn1]

To a solution of sodium hydride (4.57 g, 114.15 mmol, 60% purity) in tetrahydrofuran (600 mL) was added ethyl N-benzyl-N-(2-(1-methyl-1H-pyrazol-4-yl)acetyl)glycinate (30 g, 95.13 mmol) in tetrahydrofuran (100 mL) dropwise at 0° C. The resulting mixture was stirred at 75° C. for 16 h. After cooled to 25° C., the reaction mixture was quenched with water (500 mL). The resulting solution was concentrated under vacuum to remove tetrahydrofuran. The pH was adjusted to 6 with HCl (1 M). The solids were collected by filtration to afford 1-benzyl-3-(1-methyl-1H-pyrazol-4-yl)pyrrolidine-2,4-dione (16.5 g, 64% yield) as a white solid. ¹H NMR (300 MHz, DMSO-d₆) δ: 11.43 (br, 1H), 8.01 (s, 1H), 7.83 (s, 1H), 7.21-7.38 (m, 5H), 4.54 (s, 2H), 3.85 (s, 3H), 3.81 (s, 2H). LC-MS: m/z 270 [M+H]⁺.

### Step 5: 1-benzyl-3-(1-methyl-1H-pyrazol-4-yl)-3-(trifluoromethyl)pyrrolidine-2,4-dione

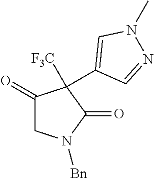 [SMILES: Cn1cc(C2(C(F)(F)F)C(=O)CN(Cc3ccccc3)C2=O)cn1]

To a stirred solution of 1-benzyl-3-(1-methyl-1H-pyrazol-4-yl)pyrrolidine-2,4-dione (75 g, 278.5 mmol) in N,N-Dimethylformamide (1000 mL) was added sodium hydride (12.25 g, 306.3 mmol, 60% in mineral oil) in batches at 0° C. The reaction mixture was stirred at 25° C. for 0.5 h. 5-(trifluoromethyl)-5H-dibenzo[b,d]thiophen-5-ium trifluoromethanesulfonate (112.33 g, 278.5 mmol) was added in batches at −55° C. The reaction mixture was stirred at −55° C. for 1 h and stirred at 25° C. for another 1 h. The reaction mixture was quenched with water (5000 mL). The resulting solution was extracted with ethyl acetate (3×5000 mL). The combined organic layers were washed with brine (3×5000 mL), dried over anhydrous sodium sulfate and concentrated under vacuum. The residue was purified by column chromatography on silica gel using 10% petroleum ether and 90% ethyl acetate as eluent to afford 1-benzyl-3-(1-methyl-1H-pyrazol-4-yl)-3-(trifluoromethyl)pyrrolidine-2,4-dione (2.2 g, 2% yield) as light-yellow oil. ¹H NMR (400 MHz, Chloroform-d) δ: 7.78 (s, 1H), 7.76 (s, 1H), 7.33-7.45 (m, 3H), 7.25-7.27 (m, 2H), 4.85 (d, J=14.8 Hz, 1H), 4.63 (d, J=14.8 Hz, 1H), 3.94 (s, 3H), 3.91 (d, J=18.0 Hz, 0.5H), 3.79 (d, J=18.0 Hz, 0.5H). LC-MS: m/z 338 [M+H]⁺.

### Step 6: 1-benzyl-4-(1-methyl-1H-pyrazol-4-yl)-4-(trifluoromethyl)pyrrolidin-3-ol

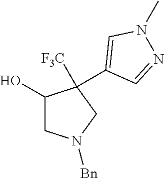 [SMILES: Cn1cc(C2(C(F)(F)F)CN(Cc3ccccc3)CC2O)cn1]

To a stirred mixture of 1-benzyl-3-(1-methyl-1H-pyrazol-4-yl)-3-(trifluoromethyl)pyrrolidine-2,4-dione (2.5 g, 7.4 mmol) in tetrahydrofuran (120 mL) was added LiAlH₄ (898 mg, 23.7 mmol) in batches at 0° C. The reaction mixture was stirred at 25° C. for 1 h. The reaction mixture was cooled to 0° C. While stirring, water (900 mg) and an aqueous solution of NaOH (10%, 900 mg) were added, followed by the addition of water (900 mg). The resulting mixture was filtered and concentrated under vacuum. to afford 1-benzyl-4-(1-methyl-1H-pyrazol-4-yl)-4-(trifluoromethyl)pyrrolidin-3-ol (2 g, crude) as yellow oil. LC-MS: m/z 326 [M+H]⁺.

### Step 7: 4-(1-methyl-1H-pyrazol-4-yl)-4-(trifluoromethyl)pyrrolidin-3-ol

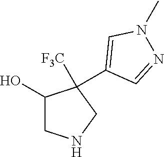 [SMILES: Cn1cc(C2(C(F)(F)F)CNCC2O)cn1]

To a stirred mixture of 1-benzyl-4-(1-methyl-1H-pyrazol-4-yl)-4-(trifluoromethyl)pyrrolidin-3-ol (2 g, 6.15 mmol) in ethanol (100 mL) was added HCl (1 N, 2 mL) and Pd/C (2 g, 10%). The reaction mixture was stirred at 25° C. for 12 h under hydrogen. The solid was filtered out. The filtrate was concentrated under vacuum to afford 4-(1-methyl-1H-pyrazol-4-yl)-4-(trifluoromethyl)pyrrolidin-3-ol (1.7 g, crude) as a yellow solid. LC-MS: m/z 236 [M+H]⁺.

### Step 8: tert-butyl 4-hydroxy-3-(1-methyl-1H-pyrazol-4-yl)-3-(trifluoromethyl)pyrrolidine-1-carboxylate

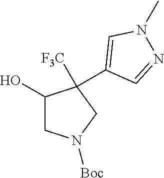 [SMILES: Cn1cc(C2(C(F)(F)F)CN(C(=O)OC(C)(C)C)CC2O)cn1]

To a stirred mixture of 4-(1-methyl-1H-pyrazol-4-yl)-4-(trifluoromethyl)pyrrolidin-3-ol (1.7 g, 7.2 mmol) in tetrahydrofuran (100 mL) were added TEA (3.66 g, 36.1 mmol) and (Boc)₂O (2.37 g, 10.8 mmol). The reaction mixture was stirred at 25° C. for 2 h. The mixture was concentrated under vacuum. The residue was purified by column chromatography on silica gel using 10% petroleum ether and 90% ethyl acetate as eluent to afford tert-butyl 4-hydroxy-3-(1-methyl-1H-pyrazol-4-yl)-3-(trifluoromethyl)pyrrolidine-1-carboxylate (1.2 g, 50% three steps yield) as yellow oil. LC-MS: m/z 336 [M+H]⁺.

### Step 9: tert-butyl 3-(1-methyl-1H-pyrazol-4-yl)-4-oxo-3-(trifluoromethyl)pyrrolidine-1-carboxylate

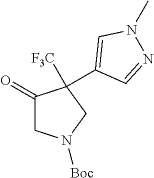 [SMILES: Cn1cc(C2(C(F)(F)F)CN(C(=O)OC(C)(C)C)CC2=O)cn1]

To a stirred mixture of tert-butyl 4-hydroxy-3-(1-methyl-1H-pyrazol-4-yl)-3-(trifluoromethyl)pyrrolidine-1-carboxylate (1.2 g, 3.6 mmol) in dichloromethane (150 mL) were added silica gel (770 mg) and PCC (771 mg, 3.6 mmol). The reaction mixture was stirred at 40° C. for 12 h. The solid was filtered out. The filtrate was concentrated under vacuum. The residue was purified by column chromatography using 60% petroleum ether and 40% ethyl acetate as eluent to afford tert-butyl 3-(1-methyl-1H-pyrazol-4-yl)-4-oxo-3-(trifluoromethyl)pyrrolidine-1-carboxylate (420 mg, 35% yield) as colorless oil. LC-MS: m/z 334 [M+H]⁺.

### Step 10: tert-butyl (E)-2-((dimethylamino)methylene)-4-(1-methyl-1H-pyrazol-4-yl)-3-oxo-4-(trifluoromethyl)pyrrolidine-1-carboxylate

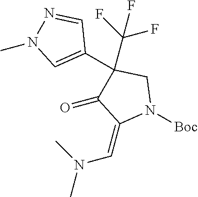 [SMILES: CN(C)/C=C1\C(=O)C(c2cnn(C)c2)(C(F)(F)F)CN1C(=O)OC(C)(C)C]

A mixture of tert-butyl 3-(1-methyl-1H-pyrazol-4-yl)-4-oxo-3-(trifluoromethyl)pyrrolidine-1-carboxylate (300 mg, 900.0 μmol) in DMF-DMA (1 mL) was stirred at 35° C. for 1 h. The reaction mixture was concentrated under vacuum to afford tert-butyl (E)-2-((dimethylamino)methylene)-4-(1-methyl-1H-pyrazol-4-yl)-3-oxo-4 (trifluoromethyl)pyrrolidine-1-carboxylate (400 mg, crude) as yellow oil. LC-MS: m/z 389 [M+H]⁺.

### Step 11: tert-butyl 2-chloro-8-(1-methyl-1H-pyrazol-4-yl)-8-(trifluoromethyl)-7,8-dihydro-6H-pyrazolo[1,5-a]pyrrolo[2,3-e]pyrimidine-6-carboxylate

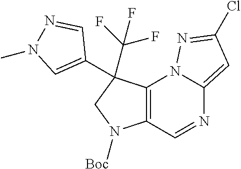 [SMILES: Cn1cc(C2(C(F)(F)F)CN(C(=O)OC(C)(C)C)c3cnc4cc(Cl)nn4c32)cn1]

To a stirred solution of tert-butyl (E)-2-((dimethylamino)methylene)-4-(1-methyl-1H-pyrazol-4-yl)-3-oxo-4-(trifluoromethyl)pyrrolidine-1-carboxylate (400 mg, 1.0 mmol) in toluene (5 mL) was added acetic acid (0.5 mL) and 3-chloro-1H-pyrazol-5-amine (121 mg, 1.0 mmol). The reaction mixture was stirred at 110° C. for 12 h. After cooled to 25° C., the mixture was concentrated under vacuum. The residue was diluted with saturated aqueous NaHCO₃ solution (80 mL). The resulting solution was extracted with ethyl acetate (3×100 mL). The combined organic layers were dried over anhydrous sodium sulfate and concentrated under vacuum. The residue was purified by column chromatography using 50% petroleum ether and 50% ethyl acetate as eluent to afford tert-butyl 2-chloro-8-(1-methyl-1H-pyrazol-4-yl)-8-(trifluoromethyl)-7,8-dihydro-6H-pyrazolo[1,5-a]pyrrolo[2,3-e]pyrimidine-6-carboxylate (80 mg, 20% two steps yield) as a yellow solid. LC-MS: m/z 443 [M+H]⁺.

### Step 12: 2-chloro-8-(1-methyl-1H-pyrazol-4-yl)-8-(trifluoromethyl)-7,8-dihydro-6H-pyrazolo[1,5-a]pyrrolo[2,3-e]pyrimidine

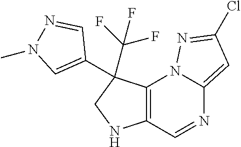 [SMILES: Cn1cc(C2(C(F)(F)F)CNc3cnc4cc(Cl)nn4c32)cn1]

To a stirred solution of tert-butyl 2-chloro-8-(1-methyl-1H-pyrazol-4-yl)-8-(trifluoromethyl)-7,8-dihydro-6H-pyrazolo[1,5-a]pyrrolo[2,3-e]pyrimidine-6-carboxylate (80 mg, 180.6 μmol) in dichloromethane (5 mL) was added TFA (2 mL). The reaction mixture was stirred at 25° C. for 1 h. The reaction mixture was concentrated under vacuum. The residue was diluted with saturated aqueous NaHCO₃ solution (40 mL). The resulting mixture was extracted with ethyl acetate (3×40 mL). The combined organic layers were dried over anhydrous sodium sulfate and concentrated under vacuum. The residue was purified by column chromatography using 95% dichloromethane and 5% methanol as eluent to afford 2-chloro-8-(1-methyl-1H-pyrazol-4-yl)-8-(trifluoromethyl)-7,8-dihydro-6H-pyrazolo[1,5-a]pyrrolo[2,3-e]pyrimidine (50 mg, 80% yield) as yellow solid. ¹H NMR (300 MHz, Chloroform-d) δ: 8.32 (s, 1H), 7.82 (s, 1H), 7.56 (s, 1H), 6.71 (s, 1H), 4.31 (d, J=11.4 Hz, 1H), 4.10 (d, J=11.4 Hz, 1H), 3.91 (s, 3H). LC-MS: m/z 343 [M+H]⁺.

### Step 13: 2-chloro-N-(6-(difluoromethyl)pyridazin-4-yl)-8-(1-methyl-1H-pyrazol-4-yl)-8-(trifluoromethyl)-7,8-dihydro-6H-pyrazolo[1,5-a]pyrrolo[2,3-e]pyrimidine-6-carboxamide

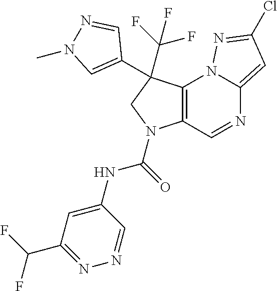 [SMILES: Cn1cc(C2(C(F)(F)F)CN(C(=O)Nc3cnnc(C(F)F)c3)c3cnc4cc(Cl)nn4c32)cn1]

To a stirred solution of 2-chloro-8-(1-methyl-1H-pyrazol-4-yl)-8-(trifluoromethyl)-7,8-dihydro-6H-pyrazolo[1,5-a]pyrrolo[2,3-e]pyrimidine (60 mg, 175.1 μmol) and 6-(difluoromethyl)pyridazine-4-carboxylic acid (Method Q2 step 8; 45 mg, 262.6 μmol) in dioxane (1 mL) was added TEA (53 mg, 525.2 μmol) and DPPA (51 mg, 210.1 μmol). The reaction mixture was stirred at 110° C. for 2 h. After cooled to 25° C., the mixture was concentrated under vacuum. The residue was purified by prep-TLC using 50% petroleum ether and 50% ethyl acetate as eluent to afford 80 mg of the crude product. The crude product was submitted to Prep-HPLC purification and the collected fractions were lyophilized to give 2-chloro-N-(6-(difluoromethyl)pyridazin-4-yl)-8-(1-methyl-1H-pyrazol-4-yl)-8-(trifluoromethyl)-7,8-dihydro-6H-pyrazolo[1,5-a]pyrrolo[2,3-e]pyrimidine-6-carboxamide (53 mg, 58% yield) as white solid. ¹H NMR (300 MHz, DMSO-d₆) δ: 9.93 (br, 1H), 9.50 (d, J=2.1 Hz, 1H), 9.41 (s, 1H), 8.20 (d, J=2.1 Hz, 1H), 7.99 (s, 1H), 7.66 (s, 1H), 7.25 (t, J=54.3 Hz, 1H), 7.11 (s, 1H), 5.00 (d, J=11.7 Hz, 1H), 4.79 (d, J=11.1 Hz, 1H), 3.62 (s, 3H). LC-MS: m/z 514 [M+H]⁺.

### Step 14: Separation of enantiomers to obtain (S)-2-chloro-N-(6-(difluoromethyl)pyridazin-4-yl)-8-(1-methyl-1H-pyrazol-4-yl)-8-(trifluoromethyl)-7,8-dihydro-6H-pyrazolo[1,5-a]pyrrolo[2,3-e]pyrimidine-6-carboxamide and (R)-2-chloro-N-(6-(difluoromethyl)pyridazin-4-yl)-8-(1-methyl-1H-pyrazol-4-yl)-8-(trifluoromethyl)-7,8-dihydro-6H-pyrazolo[1,5-a]pyrrolo[2,3-e]pyrimidine-6-carboxamide

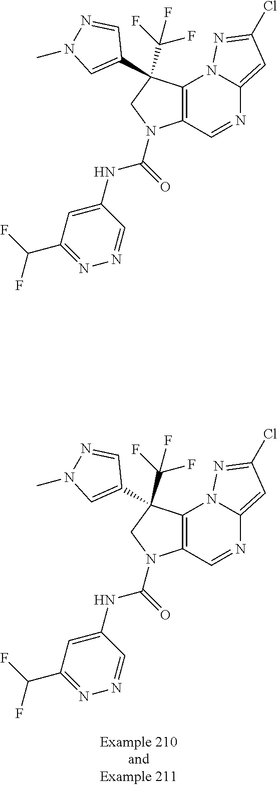 [SMILES: Cn1cc([C@]2(C(F)(F)F)CN(C(=O)Nc3cnnc(C(F)F)c3)c3cnc4cc(Cl)nn4c32)cn1 ; Cn1cc([C@@]2(C(F)(F)F)CN(C(=O)Nc3cnnc(C(F)F)c3)c3cnc4cc(Cl)nn4c32)cn1]

2-chloro-N-(6-(difluoromethyl)pyridazin-4-yl)-8-(1-methyl-1H-pyrazol-4-yl)-8-(trifluoromethyl)-7,8-dihydro-6H-pyrazolo[1,5-a]pyrrolo[2,3-e]pyrimidine-6-carboxamide (50 mg, 97.3 μmol) was submitted to chiral-HPLC: Column: CHIRAL ART Cellulose-SC, 2×25 cm, 5 μm; Mobile Phase A: Hex(0.1% FA)-HPLC, Mobile Phase B: EtOH-HPLC; Flow rate: 20 mL/min; Gradient: 30% B to 30% B in 13.25 min; Wave Length: 220/254 nm; RT1 (min): 9.79; RT2 (min): 12.16; Sample Solvent: MeOH:DCM=1:1; Injection Volume: 1 mL; Number Of Runs: 3. The first eluting isomer was concentrated and lyophilized to afford Example 210 (18.0 mg, 36% yield) as a white solid. The second eluting isomer was concentrated and lyophilized to afford Example 211 (17.3 mg, 34% yield) as a white solid. Examples 210 and 211 are enantiomers, but their absolute stereochemistry is not yet known.

Example 210: ¹H NMR (300 MHz, DMSO-d₆) δ: 10.01 (br, 1H), 9.42-9.44 (m, 2H), 8.19 (d, J=2.4 Hz, 1H), 7.98 (s, 1H), 7.66 (s, 1H), 7.22 (t, J=54.3 Hz, 1H), 7.09 (s, 1H), 5.00 (d, J=11.7 Hz, 1H), 4.75 (d, J=11.4 Hz, 1H), 3.82 (s, 3H). LC-MS: m/z 514 [M+H]⁺.

Example 211: ¹H NMR (300 MHz, DMSO-d₆) δ: 10.01 (br, 1H), 9.42-9.45 (m, 2H), 8.19 (d, J=2.4 Hz, 1H), 7.99 (s, 1H), 7.66 (s, 1H), 7.22 (t, J=54.3 Hz, 1H), 7.09 (s, 1H), 5.00 (d, J=11.7 Hz, 1H), 4.75 (d, J=11.7 Hz, 1H), 3.82 (s, 3H). LC-MS: m/z 514 [M+H]⁺.

### Biological Assays

### MALT1 Protease Assays

MALT1 protease activity was assessed in an in vitro assay using a tetrapeptide as substrate and full-length MALT1 protein His-MALT1(1-824) purified from baculovirus-infected insect cells. The tetrapeptide substrate is Ac-LRSR-AMC (SM Biochemicals) with Kₘ measured at around 100 μM.

The final assay buffer includes 1 nM (Assay 2) or 2 nM (Assay 1) of MALT1 full-length protein, 50 μM Ac-LRSR-AMC substrate, 50 mM Tris pH 7.5, 600 mM Sodium Citrate, 1 mM DTT, 1 mM EDTA, and 0.05% BSA in 384-well plate format using black microtiter square well plates (Optiplate 384-F, Perkin Elmer).

Test compounds were dissolved in 100% DMSO at stock of 10 mM, with final DMSO concentration 0.1%. Test compounds were pre-incubated with MALT1 protein for 2 h at room temperature. Substrate was added after the pre-incubation and fluorescence signal was measured using Envision at excitation 355 nm and emission 460 nm after 8 hr incubation at RT. Increase in the assay signal was linear over this period and proportional with increase in the enzyme content.

The fluorescence units were transformed to percentage of remaining activity by using the high control (HC, median of fluorescence signal from wells containing MALT1 protein, substrate, and DMSO) and low control (LC, median of fluorescence signal from wells with substrate only) with the formula below:

$$\% Remaining Activity = 100 \% \times \frac{{Signal}_{Sample} - {Signal}_{LC}}{{Signal}_{HC} - {Signal}_{LC}}$$

IC₅₀ and Hill coefficient were obtained using Graph Pad Prism (Graph Pad software, Inc, USA) with non-linear regression analysis. MALT1 inhibition IC₅₀ values of certain compounds described herein are provided in Table A below.

### Human IL10 Secretion Assays

IL10 is one of the cytokines that are regulated via activation of NF-kB signaling. For example, in ABC-DLBCL cell lines, the activated NF-kB signaling leads to increased IL10 secretion. Inhibition of NF-kB signaling has been shown leading to decreased IL10 secretion.

Assay 1: OCI-LY10 cells were seeded in IMEM supplemented with 20% fetal bovine serum at 3×10⁵ cells per well in 96-well round bottom plates (Corning 3799, Corning), treated with 100 nL of 3-fold serial compound dilutions, starting at 10 mM. The final vehicle concentration was 0.1% DMSO in all wells. After 24 h incubation, the cells were transferred to 96-PCR plates (Axygen: PCR-96-FLT-C) and centrifuged, then 16 μL of cell culture media was transferred to HTRF plates and IL10 levels were measured using human IL-10 Assay Kits (Cisbio) using HTRF format. The signals were transformed to percentage of remaining activity by using the high control (HC, median of signal from wells containing cells treated with DMSO) and low control (LC, median of signal from wells with no cells). IC50 values (nM) were determined using 4-parametric curve-fitting and Hill coefficient were obtained using Graph Pad Prism (Graph Pad software, Inc, USA) with non-linear regression analysis.

Assay 2: OCI-LY10 cells were seeded in IMEM supplemented with 20% fetal bovine serum at 4.8×10⁵ cells per 160 μL per well in 96-well V-bottom cell culture plates (coming, 3894), treated with 120 nL of 3-fold serial compound dilutions, starting at 4 mM. The final vehicle concentration was 0.075% DMSO in all wells. After a 24 h incubation, human IL-10 pre-coated plates (Meso Scale Discovery) were washed 3 times with PBST, and 50 μL culture medium was aspirated into the MSD plate and incubated at 4° C. overnight. The supernatant was then discarded and the wells were washed 3 times with PBST. SULFO-TAG Anti-human IL-10 Antibody (50×) was diluted 50 fold according to the Meso Scale Protocol, then 25 μL of SULFO-TAG Anti-human IL-10 Antibody (1×) was added. After 2 h of incubation at RT, the supernatant was discarded and the well was washed 3 times with PBST. 2× read buffer was added and the signal was read on an MSD Sector S600. The effect of a particular compound on IL10 secretion is shown relative to the effect of DMSO; set as 100%. IC₅₀ values (nM) were determined using 4-parametric curve-fitting.

The biological activity of certain compounds using the assays described above is shown in Table A. The MALT1 IC₅₀ and IL10 secretion cell assay IC₅₀ ranges are as follows: A denotes IC₅₀<10 nM; B denotes 10 nM≤IC₅₀<100 nM; C denotes 100 nM≤IC₅₀<1000 nM; D denotes IC₅₀≥1000 nM. NA denotes value not determined with that assay for the specified compound.

TABLE A
IC₅₀ Values for Selected Compounds of Formula (I)

| | MALT1 | MALT1 | IL-10 Secretion | IL-10 Secretion |
|---|---|---|---|---|
| Ex. | IC₅₀ [nM] | IC₅₀ [nM] | IC₅₀ [nM] | IC₅₀ [nM] |
| No. | Assay 1 | Assay 2 | Assay 1 | Assay 2 |
| 1 | B | B | B | B |
| 2 | A | NA | C | NA |
| 3 | B | NA | C | NA |
| 4 | A | B | B | NA |
| 5 | A | NA | C | NA |
| 6 | B | NA | C | NA |
| 7 | A | NA | B | NA |
| 8 | B | NA | C | NA |
| 9 | B | NA | D | NA |
| 10 | A | NA | C | NA |
| 11 | B | NA | NA | NA |
| 12 | A | NA | B | NA |
| 13 | B | NA | C | NA |
| 14 | C | NA | NA | NA |
| 15 | B | NA | NA | NA |
| 16 | A | A | B | B |
| 17 | B | NA | NA | NA |
| 18 | A | A | B | NA |
| 19 | A | NA | C | NA |
| 20 | B | NA | C | NA |
| 21 | A | A | B | NA |
| 22 | B | NA | NA | NA |
| 23 | B | NA | NA | NA |
| 24 | A | A | B | NA |
| 25 | A | A | B | NA |
| 26 | A | A | B | NA |
| 27 | A | A | B | NA |
| 28 | B | NA | NA | NA |
| 29 | A | B | B | NA |
| 30 | B | NA | NA | NA |
| 31 | B | NA | NA | NA |
| 32 | A | A | B | NA |
| 33 | B | NA | C | NA |
| 34 | A | NA | B | NA |
| 35 | A | A | B | B |
| 36 | A | A | B | NA |
| 37 | C | C | NA | NA |
| 38 | B | NA | C | NA |
| 39 | A | A | B | B |
| 40 | A | A | B | B |
| 41 | A | A | B | NA |
| 42 | B | B | C | NA |
| 43 | B | NA | C | NA |
| 44 | A | NA | B | NA |
| 45 | B | NA | B | NA |
| 46 | B | NA | NA | NA |
| 47 | B | NA | C | NA |
| 48 | A | NA | B | NA |
| 49 | A | NA | B | B |
| 50 | B | NA | NA | NA |
| 51 | B | NA | NA | NA |
| 52 | A | NA | B | NA |
| 53 | NA | C | NA | NA |
| 54 | D | C | NA | NA |
| 55 | NA | B | NA | NA |
| 56 | NA | C | NA | NA |
| 57 | NA | A | B | NA |
| 58 | NA | B | NA | NA |
| 59 | NA | B | B | NA |
| 60 | NA | A | B | NA |
| 61 | NA | B | C | NA |
| 62 | NA | B | B | NA |
| 63 | NA | A | B | NA |
| 64 | NA | B | B | NA |
| 65 | NA | B | NA | B |
| 66 | NA | A | C | NA |
| 67 | NA | C | NA | NA |
| 68 | NA | B | NA | C |
| 69 | NA | B | NA | B |
| 70 | NA | A | C | NA |
| 71 | NA | A | C | NA |
| 72 | NA | B | C | NA |
| 73 | NA | B | B | NA |
| 74 | NA | B | B | NA |
| 75 | NA | B | C | NA |
| 76 | NA | A | B | NA |
| 77 | NA | A | B | NA |
| 78 | NA | C | NA | NA |
| 79 | NA | A | B | NA |
| 80 | NA | C | NA | NA |
| 81 | NA | C | NA | NA |
| 82 | NA | B | C | NA |
| 83 | NA | A | C | NA |
| 84 | NA | B | C | NA |
| 85 | NA | A | C | NA |
| 86 | NA | A | NA | B |
| 87 | NA | A | NA | B |
| 88 | NA | B | NA | B |
| 89 | NA | C | NA | NA |
| 90 | NA | B | NA | NA |
| 91 | NA | A | NA | B |
| 92 | NA | A | NA | B |
| 93 | NA | B | NA | B |
| 94 | NA | C | NA | NA |
| 95 | NA | B | NA | B |
| 96 | NA | B | NA | C |
| 97 | NA | A | NA | B |
| 98 | NA | B | NA | C |
| 99 | NA | C | NA | NA |
| 100 | NA | C | NA | NA |
| 101 | NA | C | NA | NA |
| 102 | NA | C | NA | NA |
| 103 | NA | C | NA | NA |
| 104 | NA | A | B | B |
| 105 | NA | C | NA | NA |
| 106 | NA | B | NA | NA |
| 107 | NA | B | NA | NA |
| 108 | NA | D | NA | NA |
| 109 | NA | C | NA | NA |
| 110 | NA | C | NA | NA |
| 111 | NA | B | B | B |
| 112 | NA | C | NA | NA |
| 113 | NA | B | C | B |
| 114 | NA | D | NA | NA |
| 115 | NA | B | B | B |
| 116 | NA | A | B | B |
| 117 | NA | C | NA | NA |
| 118 | NA | B | NA | NA |
| 119 | NA | B | NA | NA |
| 120 | NA | D | NA | NA |
| 121 | NA | B | NA | NA |
| 122 | NA | A | B | B |
| 123 | NA | B | B | B |
| 124 | NA | D | NA | NA |
| 125 | NA | A | B | B |
| 126 | NA | A | B | B |
| 127 | NA | A | B | B |
| 128 | NA | A | B | B |
| 129 | NA | B | NA | NA |
| 130 | NA | C | NA | NA |
| 131 | NA | B | NA | NA |
| 132 | NA | C | NA | NA |
| 133 | NA | B | C | B |
| 134 | NA | B | C | B |
| 135 | NA | C | NA | NA |
| 136 | NA | A | B | B |
| 137 | NA | D | NA | NA |
| 138 | NA | B | NA | NA |
| 139 | NA | D | NA | NA |
| 140 | NA | A | C | B |
| 141 | NA | D | NA | NA |
| 142 | NA | B | NA | NA |
| 143 | NA | C | NA | NA |
| 144 | NA | B | C | B |
| 145 | NA | C | NA | NA |
| 146 | NA | A | C | C |
| 147 | NA | D | NA | NA |
| 148 | NA | C | NA | NA |
| 149 | NA | A | B | B |
| 150 | NA | A | B | B |
| 151 | NA | A | B | B |
| 152 | NA | A | B | B |
| 153 | NA | B | D | C |
| 154 | NA | C | NA | NA |
| 155 | NA | C | NA | NA |
| 156 | NA | D | NA | NA |
| 157 | NA | C | NA | NA |
| 158 | NA | D | NA | NA |
| 159 | NA | B | NA | NA |
| 160 | NA | D | NA | NA |
| 161 | NA | C | NA | NA |
| 162 | NA | B | NA | NA |
| 163 | NA | D | NA | NA |
| 164 | NA | C | NA | NA |
| 165 | NA | D | NA | NA |
| 166 | NA | D | NA | NA |
| 167 | NA | D | NA | NA |
| 168 | NA | D | NA | NA |
| 169 | NA | D | NA | NA |
| 170 | NA | D | NA | NA |
| 171 | NA | D | NA | NA |
| 172 | NA | C | NA | NA |
| 173 | NA | D | NA | NA |
| 174 | NA | D | NA | NA |
| 175 | NA | D | NA | NA |
| 176 | NA | D | NA | NA |
| 177 | NA | D | NA | NA |
| 178 | NA | D | NA | NA |
| 179 | NA | D | NA | NA |
| 180 | NA | C | NA | NA |
| 181 | NA | A | B | B |
| 182 | NA | D | NA | NA |
| 183 | NA | B | B | B |
| 184 | NA | B | B | B |
| 185 | NA | D | NA | NA |
| 186 | NA | D | NA | NA |
| 187 | NA | D | NA | NA |
| 188 | NA | B | C | B |
| 189 | NA | C | NA | NA |
| 190 | NA | D | NA | NA |
| 191 | NA | A | B | B |
| 192 | NA | D | NA | NA |
| 193 | NA | B | NA | NA |
| 194 | NA | D | NA | NA |
| 195 | NA | B | NA | NA |
| 196 | NA | D | NA | NA |
| 197 | NA | D | NA | NA |
| 198 | NA | B | NA | NA |
| 199 | NA | D | NA | NA |
| 200 | NA | D | NA | NA |
| 201 | NA | A | B | B |
| 202 | NA | B | C | B |
| 203 | NA | C | NA | NA |
| 204 | NA | B | B | B |
| 205 | NA | A | B | B |
| 206 | NA | D | NA | NA |
| 207 | NA | D | NA | NA |
| 208 | NA | D | NA | NA |
| 209 | NA | D | NA | NA |
| 210 | NA | D | NA | NA |
| 211 | NA | B | NA | NA |

What is claimed is:

1. A compound of Formula (I), or a pharmaceutically acceptable salt thereof: 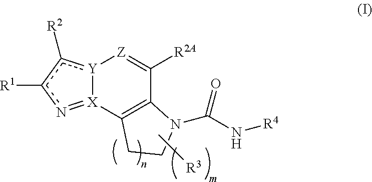
wherein:
each [symbol] is a single or double bond;
X is N or C;
Y is N or C;
Z is N or CR⁵;
wherein when one of X and Y is N, the other of X and Y is C;
n is 1, 2, or 3;
R¹ is hydrogen, halogen, cyano, hydroxyl, C1-C3 alkoxy, C1-C3 haloalkoxy, C1-C3 haloalkyl, —NR^{A}R^{B}, or C1-C3 alkyl optionally substituted with 1-3 substituents independently selected from hydroxyl and C1-C3 alkoxy;
R² is hydrogen, amino, or halogen;
R^{2A} is hydrogen, halogen, or C1-C6 alkyl;
each R³ is independently halogen, hydroxyl, cyano, C3-C6 cycloalkyl, —NR^{A}R^{B}, 5-6 membered heteroaryl optionally substituted with C1-C3 alkyl; C1-C3 alkyl optionally substituted with C1-C3 alkoxy or cyano, C1-C3 alkoxy, C1-C3 haloalkoxy, or C1-C3 haloalkyl; or two R³ together with the carbon atom to which they are attached come together to form an oxo group or a C3-C8 cycloalkyl;
m is 0, 1, 2, or 3;
R⁴ is phenyl, napthyl, 5-10 membered heteroaryl, 3-10 membered heterocyclyl, or C3-C8 cycloalkyl; wherein each R group is optionally substituted with 1-3 substituents independently selected from R⁶;
R⁵ is hydrogen, halogen, cyano, hydroxyl, C1-C3 alkoxy, C1-C3 haloalkoxy, C1-C3 haloalkyl, —NR^{C}R^{D}, or C1-C3 alkyl; and
each R⁶ is independently selected from halogen; cyano; hydroxyl; —CO₂H; —N═(S═O)(C1-C3 alkyl)₂, —S(═O)ₚ(C1-C3 alkyl), —NR^{E}R^{F}; —(C═O)NR^{E}R^{F}; C1-C3 alkoxy optionally substituted with amino, hydroxyl, or —(C═O)NR^{E}R^{F}; C1-C3 haloalkyl; C1-C3 haloalkoxy; 5-6 membered heteroaryl optionally substituted with 1-3 independently selected R^{X}; C1-C3 alkyl optionally substituted with 1-2 substituents independently selected from hydroxyl, —NR^{E}R^{F}, C1-C3 alkoxy, and C3-C6 cycloalkyl; C3-C6 cycloalkyl optionally substituted with hydroxyl; and -(Q)_{q}-3-8 membered heterocyclyl optionally substituted with 1-3 independently selected C1-C3 alkyl;
p is 1 or 2;
Q is —O— or —NH—;
q is 0 or 1;
each R^{X} is independently selected from halogen, cyano, hydroxyl, amino, C1-C3 alkoxy, C1-C3 haloalkoxy, C1-C3 haloalkyl, or C1-C6 alkyl optionally substituted with 1-3 substituents independently selected from hydroxyl, C1-C3 alkoxy, and —NR^{G}R^{H};
R^{A}, R^{B}, R^{C}, R^{D} are independently hydrogen, C1-C3 alkyl, or R^{A} and R^{B}, or R^{C} and R^{D}, together with the nitrogen atom to which they are attached come together to form a 4-6 membered heterocyclyl; and
R^{E}, R^{F}, R^{G}, and R^{H} are independently hydrogen, C1-C3 alkyl, or C3-C6 cycloalkyl, or R^{E} and R^{F}, or R^{G} and R^{H}, together with the nitrogen atom to which they are attached come together to form a 4-6 membered heterocyclyl optionally substituted with C1-C3 alkyl or C1-C3 alkoxy.

2. The compound of claim 1, wherein X is C and Y is C.

3. The compound of claim 1, wherein X is N and Y is C.

4. The compound of claim 1, wherein X is C and Y is N.

5. The compound of any one of claims 1-4, wherein Z is N.

6. The compound of any one of claims 1-4, wherein Z is CR⁵.

7. The compound of any one of claims 1-6, wherein R¹ is hydrogen.

8. The compound of any one of claims 1-6, wherein R¹ is halogen.

9. The compound of any one of claims 1-6 and 8, wherein R¹ is chloro.

10. The compound of any one of claims 1-6 and 8, wherein R¹ is fluoro.

11. The compound of any one of claims 1-6, wherein R¹ is cyano.

12. The compound of any one of claims 1-6, wherein R¹ is hydroxyl.

13. The compound of any one of claims 1-6, wherein R¹ is C1-C3 alkoxy.

14. The compound of any one of claims 1-6, wherein R¹ is C1-C3 haloalkoxy.

15. The compound of any one of claims 1-6, wherein R¹ is C1-C3 haloalkyl.

16. The compound of any one of claims 1-6, wherein R¹ is —NR^{A}R^{B}.

17. The compound of any one of claim 1 or 16, wherein R^{A} and R are independently hydrogen or C1-C3 alkyl.

18. The compound of any one of claim 1 or 16-17, wherein one of R^{A} and R^{B} is hydrogen and the other of R^{A} and R^{B} is C1-C3 alkyl.

19. The compound of any one of claim 1 or 16-17, wherein R^{A} and R are both hydrogen.

20. The compound of any one of claim 1 or 16-17, wherein R^{A} and R^{B} are both C1-C3 alkyl.

21. The compound of any one of claim 1 or 16-17, wherein R^{A} and R^{B} together with the nitrogen atom to which they are attached come together to form a 4-6 membered heterocyclyl.

22. The compound of any one of claims 1-6, wherein R¹ is C1-C3 alkyl optionally substituted with 1-3 substituents independently selected from hydroxyl and C1-C3 alkoxy.

23. The compound of any one of claims 1-6 and 22, wherein R¹ is unsubstituted C1-C3 alkyl.

24. The compound of any one of claims 1-6 and 22, wherein R¹ is C1-C3 alkyl substituted with 1-3 substituents independently selected from hydroxyl and C1-C3 alkoxy.

25. The compound of any one of claims 1-24, wherein R² is hydrogen.

26. The compound of any one of claims 1-24, wherein R² is halogen.

27. The compound of any one of claims 1-24, wherein R² is amino.

28. The compound of any one of claims 1-27, wherein R^{2A} is hydrogen.

29. The compound of any one of claims 1-27, wherein R^{2A} is halogen.

30. The compound of any one of claims 1-27, wherein R^{2A} is C1-C6 alkyl.

31. The compound of any one of claims 1-30, wherein n is 1.

32. The compound of any one of claims 1-30, wherein n is 2.

33. The compound of any one of claims 1-30, wherein n is 3.

34. The compound of any one of claims 1-33, wherein m is 1.

35. The compound of any one of claims 1-33, wherein m is 2.

36. The compound of any one of claims 1-33, wherein m is 3.

37. The compound of any one of claims 1-36, wherein each R³ is independently halogen, cyano, C3-C6 cycloalkyl, C1-C3 alkyl optionally substituted with C1-C3 alkoxy or cyano, C1-C3 haloalkyl, C1-C3 alkoxy, or C1-C3 haloalkoxy.

38. The compound of any one of claims 1-37, wherein each R³ is independently C3-C6 cycloalkyl, C1-C3 alkyl optionally substituted with C1-C3 alkoxy or cyano, C1-C3 haloalkyl, C1-C3 alkoxy, or C1-C3 haloalkoxy.

39. The compound of any one of claims 1-38, wherein each R³ is independently unsubstituted C1-C3 alkyl or C1-C3 haloalkyl.

40. The compound of any one of claims 1-38, wherein each R³ is independently cyclopropyl, methyl optionally substituted with methoxy, trifluoromethyl, methoxy, or trifluoromethoxy.

41. The compound of any one of claims 1-38, wherein each R³ is independently cyclopropyl, methyl, methoxymethyl, or trifluoromethyl.

42. The compound of any one of claims 1-33, wherein m is 1 and R³ is methyl, methoxymethyl, trifluoromethyl, or cyclopropyl.

43. The compound of any one of claims 1-33, wherein m is 2 and each R³ is methyl.

44. The compound of any one of claims 1-33, wherein m is 2 and each R³ is trifluoromethyl.

45. The compound of any one of claims 1-33, wherein m is 2 and one R³ is methyl and the other R³ is trifluoromethyl.

46. The compound of any one of claims 1-33, wherein m is 2 and one R³ is methoxymethyl and the other R³ is trifluoromethyl.

47. The compound of any one of claims 1-33, wherein m is 2 and one R³ is methyl and the other R³ is cyclopropyl.

48. The compound of any one of claims 1-33, wherein m is 2 and one R³ is methoxymethyl and the other R³ is cyclopropyl.

49. The compound of any one of claims 1-33, wherein m is 2 and one R³ is trifluoromethyl and the other R³ is cyclopropyl.

50. The compound of any one of claims 1-33, wherein m is 2 and one R³ is methyl and the other R³ is methoxy.

51. The compound of any one of claims 1-33, wherein m is 2 and one R³ is cyclopropyl and the other R³ is methoxy.

52. The compound of any one of claims 1-33, 35-41, or 43-51, wherein the R³ groups are geminal.

53. The compound of any one of claims 1-33, wherein m is 2 and the two R³ together with the carbon atom to which they are attached come together to form an oxo group.

54. The compound of any one of claims 1-33, wherein m is 2 and the two R³ together to form a C3-C8 cycloalkyl.

55. The compound of any one of claims 1-33, wherein m is 0.

56. The compound of any one of claims 1-55, wherein R is phenyl optionally substituted with 1-3 independently selected R⁶.

57. The compound of any one of claims 1-55, wherein R⁴ is unsubstituted phenyl.

58. The compound of any one of claims 1-55, wherein R⁴ is phenyl substituted with 1-2 independently selected R⁶.

59. The compound of any one of claims 1-55, wherein R⁴ is naphthyl optionally substituted with 1-3 independently selected R⁶.

60. The compound of any one of claims 1-55, wherein R⁴ is unsubstituted naphthyl.

61. The compound of any one of claims 1-55, wherein R⁴ is naphthyl substituted with 1-3 independently selected R⁶.

62. The compound of any one of claims 1-55, wherein R⁴ is 5-6 membered heteroaryl optionally substituted with 1-3 independently selected R⁶.

63. The compound of any one of claims 1-55, wherein R⁴ is unsubstituted 5-6 membered heteroaryl.

64. The compound of any one of claims 1-55 and 62, wherein R⁴ is 5-6 membered heteroaryl substituted with 1-3 independently selected R⁶.

65. The compound of any one of claims 1-55 or 62, wherein R⁴ is 6 membered heteroaryl substituted with 1-2 independently selected R⁶.

66. The compound of any one of claims 63-65, wherein the 5-6 membered heteroaryl is 3-pyridyl, 4-pyridyl, or 4-pyridazinyl.

67. The compound of any one of claims 63-65, wherein the 5-6 membered heteroaryl is 3-pyridyl or 4-pyridyl.

68. The compound of any one of claims 1-55, wherein R⁴ is C3-C8 cycloalkyl optionally substituted with 1-3 independently selected R⁶.

69. The compound of any one of claims 1-55, wherein R⁴ is 3-10 membered heterocyclyl optionally substituted with 1-3 independently selected R⁶.

70. The compound of any one of claims 1-55 or 69, wherein R⁴ is 3-10 membered heterocyclyl optionally substituted with 1-2 independently selected R⁶.

71. The compound of any one of claims 1-56, 58-59, 61-62, or 64-69, wherein at least one of R⁶ is halogen.

72. The compound of claim 71, wherein at least one of R⁶ is chloro.

73. The compound of any one of claims 1-56, 58-59, 61-62, or 64-69, wherein at least one of R⁶ is cyano.

74. The compound of any one of claims 1-56, 58-59, 61-62, or 64-69, wherein at least one of R⁶ is hydroxyl.

75. The compound of any one of claims 1-56, 58-59, 61-62, or 64-69, wherein at least one of R⁶ is —CO₂H.

76. The compound of any one of claims 1-56, 58-59, 61-62, or 64-69, wherein at least one of R⁶ is —N═(S═O)(C1-C3 alkyl)₂ or —S(═O)ₚ(C1-C3 alkyl).

77. The compound of any one of claims 1-56, 58-59, 61-62, 64-69, or 76, wherein p is 1.

78. The compound of any one of claims 1-56, 58-59, 61-62, 64-69, or 76, wherein p is 2.

79. The compound of any one of claims 1-56, 58-59, 61-62, or 64-69, wherein at least one of R⁶ is —NR^{E}R^{F}.

80. The compound of any one of claims 1-56, 58-59, 61-62, or 64-69, wherein at least one of R⁶ is —(C═O)NR^{E}R^{F}.

81. The compound of any one of claims 1-56, 58-59, 61-62, 64-69, or 79-80, wherein R^{E} and R^{F} are independently hydrogen or C1-C3 alkyl.

82. The compound of any one of claims 1-56, 58-59, 61-62, 64-69, or 79-81, wherein one of R^{E} and R^{F} is hydrogen and the other of R^{E} and R^{F} is C1-C3 alkyl.

83. The compound of any one of claims 1-56, 58-59, 61-62, 64-69, or 79-81, wherein R^{E} and R^{F} are both hydrogen.

84. The compound of any one of claims 1-56, 58-59, 61-62, 64-69, or 79-81, wherein R^{E} and R^{F} are both C1-C3 alkyl.

85. The compound of any one of claims 1-56, 58-59, 61-62, 64-69, or 79-80, wherein R^{E} and R^{F} together with the nitrogen atom to which they are attached come together to form a 4-6 membered heterocyclyl optionally substituted with C1-C3 alkyl or C1-C3 alkoxy.

86. The compound of any one of claims 1-56, 58-59, 61-62, or 64-69, wherein at least one of R⁶ is C1-C3 alkoxy optionally substituted with amino, hydroxyl, or —(C═O)NR^{E}R^{F}.

87. The compound of any one of claims 1-56, 58-59, 61-62, 64-69, or 86, wherein at least one of R⁶ is unsubstituted C1-C3 alkoxy.

88. The compound of any one of claims 1-56, 58-59, 61-62, 64-69, or 86, wherein at least one of R⁶ is C1-C3 alkoxy substituted with amino or hydroxyl.

89. The compound of any one of claims 1-56, 58-59, 61-62, or 64-69, wherein at least one of R⁶ is C1-C3 haloalkyl.

90. The compound of any one of claims 1-56, 58-59, 61-62, or 64-69, wherein at least one of R⁶ is difluoromethyl.

91. The compound of any one of claims 1-56, 58-59, 61-62, or 64-69, wherein at least one of R⁶ is C1-C3 haloalkoxy.

92. The compound of any one of claims 1-56, 58-59, 61-62, or 64-69, wherein at least one of R⁶ is 5-6 membered heteroaryl optionally substituted with 1-3 independently selected R^{X}.

93. The compound of any one of claims 1-56, 58-59, 61-62, 64-69, or 92, wherein each R^{X} is independently selected from cyano, hydroxyl, C1-C3 alkoxy, or C1-C6 alkyl optionally substituted with 1-3 substituents independently selected from hydroxyl, C1-C3 alkoxy, and —NR^{G}R^{H}.

94. The compound of any one of claims 1-56, 58-59, 61-62, 64-69, or 92, wherein at least one of R⁶ is unsubstituted 5-6 membered heteroaryl.

95. The compound of any one of claims 1-56, 58-59, 61-62, 64-69, or 92, wherein at least one of R⁶ is 1,2,3-triazol-2-yl.

96. The compound of any one of claims 1-56, 58-59, 61-62, or 64-69, wherein at least one of R⁶ is C1-C3 alkyl optionally substituted with 1-2 substituents independently selected from hydroxyl, —NR^{E}R^{F}, C1-C3 alkoxy, and C3-C6 cycloalkyl.

97. The compound of any one of claims 1-56, 58-59, 61-62, or 64-69, wherein at least one of R⁶ is C3-C6 cycloalkyl optionally substituted with hydroxyl.

98. The compound of any one of claims 1-56, 58-59, 61-62, or 64-69, wherein at least one of R⁶ is -(Q)_{q}-3-8 membered heterocyclyl optionally substituted with 1-3 independently selected C1-C3 alkyl.

99. The compound of any one of claims 1-56, 58-59, 61-62, 64-69, or 98, wherein q is 0.

100. The compound of any one of claims 1-56, 58-59, 61-62, 64-69, or 98, wherein q is 1.

101. The compound of any one of claims 1-56, 58-59, 61-62, 64-69, 98, or 100, wherein Q is —O—.

102. The compound of any one of claims 1-56, 58-59, 61-62, 64-69, 98, or 100, wherein Q is-NH—.

103. The compound of any one of claims 1-55, wherein R is 3-pyridyl or 4-pyridyl substituted with 1-3 independently selected R⁶.

104. The compound of any one of claims 1-55 or 103, wherein R⁴ is 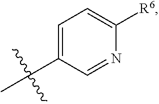
wherein the wavy line crosses the bond that connects to the —C(═O)NH— moiety of Formula (I).

105. The compound of any one of claims 1-55 or 103, wherein R⁴ is 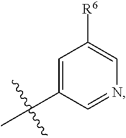
wherein the wavy line crosses the bond that connects to the —C(═O)NH— moiety of Formula (I).

106. The compound of any one of claims 1-55 or 103, wherein R⁴ is 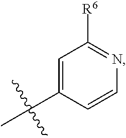
wherein the wavy line crosses the bond that connects to the —C(═O)NH— moiety of Formula (I).

107. The compound of any one of claims 103-106, wherein R⁶ is selected from the group consisting of cyano, halogen, C1-C3 haloalkyl, and C1-C3 alkoxy.

108. The compound of any one of claims 103-107, wherein R⁶ is selected from the group consisting of cyano, chloro, difluoromethyl, trifluoromethyl, and methoxy.

109. The compound of any one of claims 1-55 or 103, wherein R⁴ is 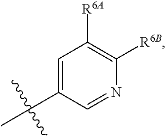
wherein the the wavy line crosses the bond that connects to the —C(═O)NH— moiety of Formula (I).

110. The compound of any one of claims 1-55 or 103, wherein R⁴ is 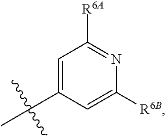
wherein the the wavy line crosses the bond that connects to the —C(═O)NH— moiety of Formula (I).

111. The compound of claim 109 or 110, wherein
R^{6A} is selected from the group consisting of: cyano, halogen, unsubstituted C1-C3 alkyl, C1-C3 alkoxy, and C1-C3 haloalkyl; and
R^{6B} is selected from the group consisting of: 5-6 membered heteroaryl optionally substituted with cyano, hydroxyl, —N═(S═O)(C1-C3 alkyl)₂, C1-C3 alkoxy, C1-C3 alkyl optionally substituted with 1-2 substituents independently selected from hydroxyl, C1-C3 alkoxy, and —NR^{G}R^{H}, or amino; —(C═O)NR^{E}R^{F}; C1-C3 alkoxy; C1-C3 haloalkyl; C1-C3 haloalkoxy; cyano; C1-C3 alkyl; and -(Q)_{q}-3-8 membered heterocyclyl optionally substituted with 1-3 independently selected C1-C3 alkyl.

112. The compound of any one of claims 109-111, wherein
R^{6A} is selected from the group consisting of: cyano, fluoro, chloro, methyl, ethyl, methoxy, difluoromethyl, trifluoromethyl; and
R^{6B} is selected from the group consisting of: 1,2,3-triazol-2-yl, 4-methyl-1,2,3-triazol-2-yl, 4-hydroxymethyl-1,2,3-triazol-2-yl, 4-(1,2-dihydroxyethyl)-1,2,3-triazol-2-yl, 4-(1-hydroxyethyl)-1,2,3-triazol-2-yl, 4-methoxymethyl-1,2,3-triazol-2-yl, 4-methyl-1,2,3-triazol-1-yl, 4-methoxy-1,2,3-triazol-2-yl, 4-amino-1,2,3-triazol-2-yl, 4-dimethylaminomethyl-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, 1,2,4-triazol-4-on-2-yl, tetrazol-5-yl, 2-methyl-tetrazol-5-yl, 1-methyl-tetrazol-5-yl, imidazol-1-yl, pyrazol-1-yl, 5-cyano-pyrazol-1-yl, 1-methyl-imidazol-3-yl, 1-methyl-5-amino-imidazol-3-yl, 3-methylimidazol-2-on-1-yl, 1-methyl-pyrazol-3-yl, 1-methyl-pyrazol-5-yl, pyrrol-1-yl, thiazol-2-yl, isothiazolidin-2-yl-1,1-dioxide, pyrrolidin-2-on-1-yl, oxazol-2-yl, oxadiazol-2-yl, 2-amino-pyrimidin-4-yl, 2-tetrahydrofuranyl, —(C═O)4-methylpiperazin-1-yl, —(C═O)N(CH₃)₂, —(C═O)NHCH₃, —N═(S═O)(methyl)₂, methoxy, ethoxy, difluoromethoxy, methyl, cyano.

113. The compound of any one of claims 109-112, wherein
R^{6A} is selected from the group consisting of: cyano, chloro, and trifluoromethyl; and
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, and 1,2,4-triazol-4-on-2-yl.

114. The compound of any one of claims 1-55 or 103, wherein R⁴ is 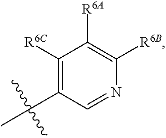
wherein the the wavy line crosses the bond that connects to the —C(═O)NH— moiety of Formula (I).

115. The compound of any one of claims 1-55 or 103, wherein R⁴ is 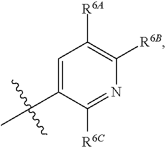
wherein the the wavy line crosses the bond that connects to the —C(═O)NH— moiety of Formula (I).

116. The compound of claim 114 or 115, wherein
R^{6A} is selected from the group consisting of: cyano, halogen, C1-C3 alkyl, C1-C3 alkoxy, and C1-C3 haloalkyl;
R^{6B} is selected from the group consisting of: 5-6 membered heteroaryl optionally substituted with cyano, C1-C3 alkyl, or amino; —(C═O)NR^{E}R^{F}; C1-C3 alkoxy; C1-C3 haloalkyl; C1-C3 haloalkoxy; cyano; and C1-C3 alkyl; and
R^{6C} is selected from the group consisting of: cyano, halogen, C1-C3 alkyl, C1-C3 alkoxy, and C1-C3 haloalkyl.

117. The compound of any one of claims 114-116, wherein
R^{6A} is selected from the group consisting of: cyano, fluoro, chloro, methyl, ethyl, methoxy, trifluoromethyl;
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, 1,2,4-triazol-4-on-2-yl, tetrazol-5-yl, 2-methyl-tetrazol-5-yl, 1-methyl-tetrazol-5-yl, imidazol-1-yl, 1-methyl-imidazol-3-yl, 1-methyl-5-amino-imidazol-3-yl, 3-methylimidazol-2-on-1-yl, 1-methyl-pyrazol-3-yl, 1-methyl-pyrazol-5-yl, pyrrol-1-yl, thiazol-2-yl, isothiazolidin-2-yl-1,1-dioxide, pyrrolidin-2-on-1-yl, oxazol-2-yl, oxadiazol-2-yl, 2-amino-pyrimidin-4-yl, —(C═O)4-methylpiperazin-1-yl, —(C═O)N(CH₃)₂, —(C═O)NHCH₃, methoxy, ethoxy, difluoromethoxy, methyl, cyano; and
R^{6C} is selected from the group consisting of: cyano, fluoro, chloro, methyl, ethyl, methoxy, methyl, and trifluoromethyl.

118. The compound of any one of claims 114-117, wherein
R^{6A} is selected from the group consisting of: cyano, chloro, and trifluoromethyl;
R^{6B} is selected from the group consisting of: 1,2,3-triazol-2-yl, 4-methyl-1,2,3-triazol-2-yl, 4-methyl-1,2,3-triazol-1-yl, 4-amino-1,2,3-triazol-2-yl, 5-cyano-1,2,3-triazol-1-yl, 1,2,3-triazol-1-yl, 3-methyl-1,2,4-triazol-1-yl, 5-methyl-1,2,4-triazol-1-yl, 5-amino-1,2,4-triazol-1-yl, 1-methyl-5-amino-1,2,4-triazol-3-yl, and 1,2,4-triazol-4-on-2-yl; and
R^{6C} is selected from the group consisting of: cyano, chloro, methyl, and trifluoromethyl.

119. The compound of any one of claims 1-4 or 6-118, wherein R⁵ is hydrogen.

120. The compound of any one of claims 1-4 or 6-118, wherein R⁵ is halogen.

121. The compound of any one of claims 1-4, 6-118, or 120, wherein the halogen is fluoro.

122. The compound of any one of claims 1-4 or 6-118, wherein R⁵ is cyano.

123. The compound of any one of claims 1-4 or 6-118, wherein R⁵ is hydroxyl.

124. The compound of any one of claims 1-4 or 6-118, wherein R⁵ is C1-C3 alkoxy.

125. The compound of any one of claims 1-4 or 6-118, wherein R⁵ is C1-C3 haloalkoxy.

126. The compound of any one of claims 1-4 or 6-118, wherein R⁵ is C1-C3 haloalkyl.

127. The compound of any one of claims 1-4 or 6-118, wherein R⁵ is —NR^{C}R^{D}.

128. The compound of any one of claims 1-4, 6-118, or 127, wherein R^{C} and R^{D} a independently hydrogen or C1-C3 alkyl.

129. The compound of any one of claims 1-4, 6-118, or 127-128, wherein one of R^{C} and R^{D} is hydrogen and the other of R^{C} and R^{D} is C1-C3 alkyl.

130. The compound of any one of claims 1-4, 6-118, or 127-128, wherein R^{C} and R^{D} are both hydrogen.

131. The compound of any one of claims 1-4, 6-118, or 127-128, wherein R^{C} and R^{D} are both C1-C3 alkyl.

132. The compound of any one of claims 1-4, 6-118, or 127-128, wherein R^{C} and R^{D} together with the nitrogen atom to which they are attached come together to form a 4-6 membered heterocyclyl.

133. The compound of any one of claims 1-4 or 6-118, wherein R⁵ is C1-C3 alkyl.

134. The compound of claim 1, wherein:
X is N;
Y is C;
Z is N;
R¹ is halogen;
R² is hydrogen;
R^{2A} is hydrogen;
m is 2 and R³ is independently unsubstituted C1-C3 alkyl or C1-C3 haloalkyl;
n is 1; and
R⁴ is 5-6 membered heteroaryl optionally substituted with 1-2 substituents independently selected from C1-C3 haloalkyl and 5-6 membered heteroaryl optionally substituted with 1-3 independently selected R^{X}.

135. The compound of claim 134, wherein R¹ is chloro or fluoro.

136. The compound of any one of claims 134-135, wherein R² is hydrogen.

137. The compound of any one of claims 134-136, wherein R^{2A} is hydrogen.

138. The compound of any one of claims 134-137, wherein each R³ is geminal.

139. The compound of any one of claims 134-138, wherein one R³ is unsubstituted C1-C3 alkyl and the other R³ is C1-C3 haloalkyl.

140. The compound of any one of claims 134-139, wherein one R³ is methyl and the other R³ is trifluoromethyl.

141. The compound of any one of claims 134-140, wherein R⁴ is substituted 6 membered heteroaryl.

142. The compound of any one of claims 134-141, wherein R⁴ is 6 membered heteroaryl substituted with 1,2,3-triazolyl.

143. The compound of claim 1, wherein the compound is selected from the group consisting of the compounds in Table 1, or a pharmaceutically acceptable salt thereof.

144. A pharmaceutical composition comprising a compound of any one of claims 1-143, or a pharmaceutically acceptable salt thereof, and at least one pharmaceutically acceptable excipient.

145. A process of preparing a compound of any one of claims 1-143, comprising:
reacting a compound of Formula (I-A) 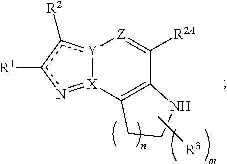
with R⁴—NH₂;
to form the compound of any one of claims 1-143.

146. The process of claim 145, wherein reacting the compound of Formula (I-A) with R⁴—NH₂ comprises reacting one of the compounds of Formula (I-A) and R⁴—NH₂ with a carbonyl equivalent selected from triphosgene and bis(trichloromethyl)carbonate to form an intermediate, then reacting the other of the compound of Formula (I-A) and R⁴—NH₂ with the intermediate.

147. The process of claim 146, comprising reacting R⁴—NH₂ with a carbonyl equivalent selected from triphosgene and bis(trichloromethyl)carbonate to form the intermediate, then reacting the compound of Formula (I-A) with the intermediate.

148. The process of any one of claims 145-147, wherein the carbonyl equivalent is triphosgene.

149. The process of any one of claims 145-147, wherein the carbonyl equivalent is bis(trichloromethyl)carbonate.

150. The process of any one of claims 145-149, wherein the compound of Formula (I-A) is a compound of Formula (I-A-N):

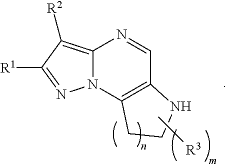

151. The process of claim 150, comprising reacting a compound of Formula (I-A-N-i) 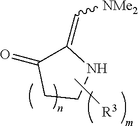
with a compound of Formula (I-A-N-ii) 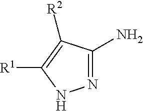
to form the compound of Formula (I-A-N).

152. The process of claim 151, wherein reacting the compound of Formula (I-A-N-i) with the compound of Formula (I-A-N-ii) is performed in the presence of acid.

153. The process of claim 152, wherein the acid is hydrochloric acid or acetic acid.

154. The process of any one of claims 145-149, wherein the compound of Formula (I-A) is a compound of Formula (I-A-M):

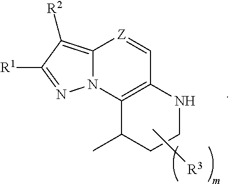

155. The process of claim 154, comprising reacting a compound of Formula (I-A-M-i) 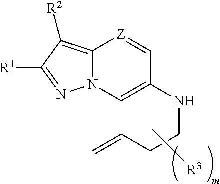
to form the compound of Formula (I-A-M).

156. The process of claim 155, wherein the compound of Formula (I-A-M-i) is reacted with an iron salt, a silane, a peroxide, and an acid to form the compound of Formula (I-A-M).

157. The process of claim 156, wherein the iron salt is ferric (Z)-4-oxopent-2-en-2-olate.

158. The process of any one of claims 155-157, wherein the silane is phenylsilane.

159. The process of any one of claims 155-158, wherein the peroxide is 2-tert-butylperoxy-2-methyl-propane.

160. The process of any one of claims 155-159, wherein the acid is 2,2,2-trifluoroacetic acid.

161. A method for treating cancer in a subject in need thereof, comprising administering to the subject an effective amount of a compound of any one of claims 1-143 or a pharmaceutically acceptable salt thereof, or a pharmaceutical composition of claim 145.

162. A method for treating a CBM complex pathway-associated cancer in a subject in need thereof, comprising administering to the subject an effective amount of a compound of any one of claims 1-143 or a pharmaceutically acceptable salt thereof, or a pharmaceutical composition of claim 145.

163. A method for treating a cancer in a subject in need thereof, comprising:
(a) identifying the cancer as being a CBM complex pathway-associated cancer; and
(b) administering to the subject an effective amount of a compound of any one of claims 1-143 or a pharmaceutically acceptable salt thereof, or a pharmaceutical composition of claim 145.

164. The method of claim 163, wherein the step of identifying the cancer in the subject as a CBM complex pathway-associated cancer includes performing an assay to detect dysregulation in a CBM complex pathway-associated gene, a CBM complex pathway-associated protease protein, or expression or activity or level of any of the same in a sample from the subject.

165. The method of claim 163 or 164, further comprising obtaining a sample from the subject.

166. The method of claim 165, wherein the sample is a biopsy sample.

167. The method of any one of claims 164-166, wherein the assay is selected from the group consisting of sequencing, immunohistochemistry, enzyme-linked immunosorbent assay, and fluorescence in situ hybridization (FISH).

168. The method of claim 167, wherein the sequencing is pyrosequencing or next generation sequencing.

169. A method for treating a cancer in a subject in need thereof, comprising:
administering to the subject an effective amount of a compound of any one of claims 1-143 or a pharmaceutically acceptable salt thereof, or a pharmaceutical composition of claim 145 to a subject identified as having a CBM complex pathway-associated cancer.

170. The method of any one of claims 162-169, wherein the CBM complex pathway-associated cancer is selected from the group consisting of a CBM complex pathway cell surface receptor-associated cancer, a cancer associated with a signal transducer between a cell surface receptor and a CBM complex, a component of a CBM complex-associated cancer, a MALT1 protease substrate-associated cancer, a cancer associated with a component of the NF-κB pathway downstream of a CBM complex, a cancer associated with a component of the JNK pathway downstream of a CBM complex, and a combination thereof.

171. The method of claim 170, wherein the CBM complex pathway cell surface receptor-associated cancer is selected from the group consisting of a CD28-associated cancer, a BCR-associated cancer, a HER1-associated cancer, a HER2-associated cancer, and combinations thereof.

172. The method of claim 170, wherein the cancer associated with a signal transducer between a cell surface receptor and a CBM complex is a protein kinase C beta (PKCβ)-associated cancer, a protein kinase C theta (PCKθ)-associated cancer, or a combination thereof.

173. The method of claim 170, wherein the component of a CBM complex-associated cancer is selected from the group consisting of a MALT1-associated cancer, a CARD11-associated cancer, a CARD14-associated cancer, a CARD10-associated cancer, a CARD9-associated cancer, a BCL10-associated cancer, and combinations thereof.

174. The method of claim 170, wherein the component of a CBM complex-associated cancer is selected from the group consisting of a MALT1-associated cancer, a CARD11-associated cancer, a BCL10-associated cancer, and combinations thereof.

175. The method of claim 170, wherein the MALT1 protease substrate-associated cancer is selected from the group consisting of a BCL10-associated cancer, an A20-associated cancer, a CYLD-associated cancer, a RelB-associated cancer, a Regnase 1-associated cancer, a roquin-1-associated cancer, a HOIL1-associated cancer, a NIK associated cancer, a LIMA1α-associated cancer, and a combination thereof.

176. The method of claim 170, wherein the MALT1 protease substrate-associated cancer is selected from the group consisting of a BCL10-associated cancer, an A20-associated cancer, a CYLD-associated cancer, and combinations thereof.

177. The method of claim 170, wherein the cancer associated with a component of the NF-κB pathway downstream of a CBM complex is selected from the group consisting of a TAK1-associated cancer, a TRAF6-associated cancer, a TAB1-associated cancer, a TAB2-associated cancer, a TAB3-associated cancer, a MKK7-associated cancer, an IKKα-associated cancer, an IKKβ-associated cancer, an IKKγ-associated cancer, an IkBα-associated cancer, a p50-associated cancer, a p65 (RelA)-associated cancer, a c-Rel-associated cancer, and combinations thereof.

178. The method of claim 170, wherein the cancer associated with a component of the NF-κB pathway downstream of a CBM complex is an IKKγ-associated cancer.

179. The method of claim 170, wherein the cancer associated with a component of the JNK pathway downstream of a CBM complex is selected from the group consisting of a JNK1-associated cancer, a JNK2-associated cancer, a JNK3-associated cancer, a MYD88 transcription factor-associated cancer, an AP-1 transcription factor-associated cancer, and combinations thereof.

180. The method of any one of claims 162-169, wherein the CBM complex pathway-associated cancer is a MALT1-associated cancer.

181. The method of claim 180, wherein the MALT1-associated cancer comprises an IAP2-MALT1 fusion.

182. The method of claim 180, wherein the MALT1-associated cancer comprises an IGH-MALT1 fusion.

183. A method of treating a MALT1-associated cancer in a subject, comprising administering to a subject identified or diagnosed as having a MALT1-associated cancer an effective amount of a compound of any one of claims 1-143 or a pharmaceutically acceptable salt thereof, or a pharmaceutical composition of claim 145.

184. A method for treating cancer in a subject in need thereof, comprising:
(a) determining that the cancer is associated with a dysregulation of a MALT1 gene, a MALT1 protease, or expression or activity or level of any of the same; and
(b) administering to the subject an effective amount of a compound of any one of claims 1-143 or a pharmaceutically acceptable salt thereof, or a pharmaceutical composition of claim 145.

185. The method of claim 184, wherein the step of determining that the cancer in the subject is a MALT1-associated cancer includes performing an assay to detect dysregulation in a MALT1 gene, a MALT1 protease protein, or expression or activity or level of any of the same in a sample from the subject.

186. The method of claim 184 or 185, further comprising obtaining a sample from the subject.

187. The method of claim 186, wherein the sample is a biopsy sample.

188. The method of any one of claims 185-187, wherein the assay is selected from the group consisting of sequencing, immunohistochemistry, enzyme-linked immunosorbent assay, and fluorescence in situ hybridization (FISH).

189. The method of claim 188, wherein the sequencing is pyrosequencing or next generation sequencing.

190. A method for inhibiting metastasis in a subject having a cancer in need of such treatment, comprising administering to the subject an effective amount of a compound of any one of claims 1-143 or a pharmaceutically acceptable salt thereof, or a pharmaceutical composition of claim 145.

191. The method of claim 190, wherein the cancer is a CBM complex pathway-associated cancer.

192. The method of claim 191, wherein the CBM complex pathway-associated cancer is selected from the group consisting of a CBM complex pathway cell surface receptor-associated cancer, a cancer associated with a signal transducer between a cell surface receptor and a CBM complex, a component of a CBM complex-associated cancer, a MALT1 protease substrate-associated cancer, a cancer associated with a component of the NF-κB pathway downstream of a CBM complex, a cancer associated with a component of the JNK pathway downstream of a CBM complex, and a combination thereof.

193. The method of claim 192, wherein the CBM complex pathway cell surface receptor-associated cancer is selected from the group consisting of a CD28-associated cancer, a BCR-associated cancer, a HER1-associated cancer, a HER2-associated cancer, and combinations thereof.

194. The method of claim 192, wherein the cancer associated with a signal transducer between a cell surface receptor and a CBM complex is a protein kinase C beta (PKCβ)-associated cancer, a protein kinase C theta (PCKθ)-associated cancer, or a combination thereof.

195. The method of claim 192, wherein the component of a CBM complex-associated cancer is selected from the group consisting of a MALT1-associated cancer, a CARD11-associated cancer, a CARD14-associated cancer, a CARD10-associated cancer, a CARD9-associated cancer, a BCL10-associated cancer, and combinations thereof.

196. The method of claim 192, wherein the component of a CBM complex-associated cancer is selected from the group consisting of a MALT1-associated cancer, a CARD11-associated cancer, a BCL10-associated cancer, and combinations thereof.

197. The method of claim 192, wherein the MALT1 protease substrate-associated cancer is selected from the group consisting of a BCL10-associated cancer, an A20-associated cancer, a CYLD-associated cancer, a RelB-associated cancer, a Regnase 1-associated cancer, a roquin-1-associated cancer, a HOIL1-associated cancer, a NIK associated cancer, a LIMA1α-associated cancer, and a combination thereof.

198. The method of claim 192, wherein the MALT1 protease substrate-associated cancer is selected from the group consisting of a BCL10-associated cancer, an A20-associated cancer, a CYLD-associated cancer, and combinations thereof.

199. The method of claim 192, wherein the cancer associated with a component of the NF-κB pathway downstream of a CBM complex is selected from the group consisting of a TAK1-associated cancer, a TRAF6-associated cancer, a TAB1-associated cancer, a TAB2-associated cancer, a TAB3-associated cancer, a MKK7-associated cancer, an IKKα-associated cancer, an IKKβ-associated cancer, an IKKγ-associated cancer, an IkBα-associated cancer, a p50-associated cancer, a p65 (RelA)-associated cancer, a c-Rel-associated cancer, and combinations thereof.

200. The method of claim 192, wherein the cancer associated with a component of the NF-κB pathway downstream of a CBM complex is an IKKγ-associated cancer.

201. The method of claim 192, wherein the cancer associated with a component of the JNK pathway downstream of a CBM complex is selected from the group consisting of a JNK1-associated cancer, a JNK2-associated cancer, a JNK3-associated cancer, a MYD88 transcription factor-associated cancer, an AP-1 transcription factor-associated cancer, and combinations thereof.

202. The method of claim 191, wherein the CBM complex pathway-associated cancer is a MALT1-associated cancer.

203. The method of claim 202, wherein the MALT1-associated cancer comprises an IAP2-MALT1 fusion.

204. The method of claim 202, wherein the MALT1-associated cancer comprises an IGH-MALT1 fusion.

205. The method of any one of claims 161-204, further comprising administering an additional therapy or therapeutic agent to the subject.

206. The method of claim 205, wherein the additional therapy or therapeutic agent is selected from radiotherapy, cytotoxic chemotherapeutics, protease-targeted therapeutics, kinase-targeted therapeutics, apoptosis modulators, signal transduction inhibitors, immune-targeted therapies, and angiogenesis-targeted therapies.

207. The method of claim 205 or 206, wherein the compound of any one of claims 1-143 or a pharmaceutically acceptable salt thereof, or a pharmaceutical composition of claim 145, and the additional therapeutic agent are administered simultaneously as separate dosages.

208. The method of claim 205 or 206, wherein the compound of any one of claims 1-143 or a pharmaceutically acceptable salt thereof, or a pharmaceutical composition of claim 145, and the additional therapeutic agent are administered as separate dosages sequentially in any order.

209. A method for treating an autoimmune disorder in a subject in need thereof, comprising administering to the subject an effective amount of a compound of any one of claims 1-143 or a pharmaceutically acceptable salt thereof, or a pharmaceutical composition of claim 145.

210. A method for treating a CBM complex pathway-associated disease or disorder in a subject in need thereof, comprising administering to the subject an effective amount of a compound of any one of claims 1-143 or a pharmaceutically acceptable salt thereof, or a pharmaceutical composition of claim 145.

211. A method for treating a disease or disorder in a subject in need thereof, comprising:
(a) identifying the cancer as being a CBM complex pathway-associated disease or disorder;
and
(b) administering to the subject an effective amount of a compound of any one of claims 1-143 or a pharmaceutically acceptable salt thereof, or a pharmaceutical composition of claim 145.

212. A method for treating a disease or disorder in a subject in need thereof, comprising:
administering to the subject an effective amount of a compound of any one of claims 1-143 or a pharmaceutically acceptable salt thereof, or a pharmaceutical composition of claim 145 to a subject identified as having a CBM complex pathway-associated disease or disorder.

213. The method of any one of claims 210-212, wherein the CBM complex pathway-associated disease or disorder is an autoimmune disease.

214. The method of any one of claims 210-212, wherein the CBM complex pathway-associated disease or disorder is an inflammatory disease.

215. The method of any one of claims 210-214, wherein the CBM complex pathway-associated cancer is selected from the group consisting of a CBM complex pathway cell surface receptor-associated cancer, a disease or disorder associated with a signal transducer between a cell surface receptor and a CBM complex, a component of a CBM complex-associated cancer, a MALT1 protease substrate-associated cancer, a disease or disorder associated with a component of the NF-κB pathway downstream of a CBM complex, a disease or disorder associated with a component of the JNK pathway downstream of a CBM complex, and a combination thereof.

216. The method of any one of claims 210-214, wherein the CBM complex pathway-associated disease or disorder is a MALT1-associated disease or disorder.

217. A method of treating a MALT1-associated autoimmune disorder in a subject, comprising administering to a subject identified or diagnosed as having a MALT1-associated autoimmune disorder an effective amount of a compound of any one of claims 1-143 or a pharmaceutically acceptable salt thereof, or a pharmaceutical composition of claim 145.

218. A method of treating a MALT1-associated autoimmune disorder in a subject, comprising administering to a subject identified or diagnosed as having a MALT1-associated autoimmune disorder an effective amount of a compound of any one of claims 1-143 or a pharmaceutically acceptable salt thereof, or a pharmaceutical composition of claim 145.

219. A method for treating an autoimmune disorder in a subject in need thereof, comprising:
(a) determining that the autoimmune disorder is associated with a dysregulation of a MALT1 gene, a MALT1 protease, or expression or activity or level of any of the same; and
(b) administering to the subject an effective amount of a compound of any one of claims 1-143 or a pharmaceutically acceptable salt thereof, or a pharmaceutical composition of claim 145.

220. A method of treating a MALT1-associated autoimmune disorder in a subject, comprising administering an effective amount of a compound of any one of claims 1-143 or a pharmaceutically acceptable salt thereof, or a pharmaceutical composition of claim 145 to a subject determined to have a MALT1-associated autoimmune disorder.

221. A method for treating an inflammatory disorder in a subject in need thereof, comprising administering to the subject an effective amount of a compound of any one of claims 1-143 or a pharmaceutically acceptable salt thereof, or a pharmaceutical composition of claim 145.

222. A method of treating a MALT1-associated inflammatory disorder in a subject, comprising administering to a subject identified or diagnosed as having a MALT1-associated inflammatory disorder an effective amount of a compound of any one of claims 1-143 or a pharmaceutically acceptable salt thereof, or a pharmaceutical composition of claim 145.

223. A method of treating a MALT1-associated inflammatory disorder in a subject, comprising administering to a subject identified or diagnosed as having a MALT1-associated inflammatory disorder an effective amount of a compound of any one of claims 1-143 or a pharmaceutically acceptable salt thereof, or a pharmaceutical composition of claim 145.

224. A method for treating an inflammatory disorder in a subject in need thereof, comprising:
(a) determining that the inflammatory disorder is associated with a dysregulation of a MALT1 gene, a MALT1 protease, or expression or activity or level of any of the same; and
(b) administering to the subject an effective amount of a compound of any one of claims 1-143 or a pharmaceutically acceptable salt thereof, or a pharmaceutical composition of claim 145.

225. A method of treating a MALT1-associated inflammatory disorder in a subject, comprising administering an effective amount of a compound of any one of claims 1-143 or a pharmaceutically acceptable salt thereof, or a pharmaceutical composition of claim 145 to a subject determined to have a MALT1-associated inflammatory disorder.

226. The method of any one of claims 209-225, further comprising administering an additional therapy or therapeutic agent to the subject.

227. The method of claim 226, wherein the additional therapy or therapeutic agent is an immunotherapy.

228. The method of claim 226 or 227, wherein the compound of any one of claims 1-143 or a pharmaceutically acceptable salt thereof, or a pharmaceutical composition according to claim 145, and the additional therapeutic agent are administered simultaneously as separate dosages.

229. The method of claim 226 or 227, wherein the compound of any one of claims 1-143 or a pharmaceutically acceptable salt thereof, or a pharmaceutical composition of claim 145, and the additional therapeutic agent are administered as separate dosages sequentially in any order.

230. A method for inhibiting mammalian cell proliferation, comprising contacting the mammalian cell with a compound of any one of claims 1-143, or a pharmaceutically acceptable salt thereof.

231. A method for inhibiting CBM complex pathway activity in a mammalian cell, comprising contacting the mammalian cell with a compound of any one of claims 1-143, or a pharmaceutically acceptable salt thereof.

232. A method for inhibiting MALT1 protease activity in a mammalian cell, comprising contacting the mammalian cell with a compound of any one of claims 1-143, or a pharmaceutically acceptable salt thereof.

233. The method of any one of claims 230-232, wherein the contacting occurs in vivo.

234. The method of any one of claims 230-232, wherein the contacting occurs in vitro.

235. The method of any one of claims 230-234, wherein the mammalian cell is a mammalian immune cell.

236. The method of any one of claims 230-235, wherein the mammalian cell is a mammalian cancer cell.

237. The method of claim 236, wherein the mammalian cancer cell is a mammalian CBM complex pathway-associated cancer cell.

238. The method of claim 236, wherein the mammalian cancer cell is a mammalian MALT1-associated cancer cell.

239. The method of any one of claims 230-238, wherein the mammalian cell has dysregulation of a MALT1 gene, a MALT1 protease protein, or expression or activity or level of any of the same.

240. The method of claim 239, wherein the dysregulation of a MALT1 gene, a MALT1 protease protein, or expression or activity or level of any of the same is an IAP2-MALT1 fusion, an IGH-MALT1 fusion, or a combination thereof.

241. Use of a compound of any one of claims 1-143 or a pharmaceutically acceptable salt thereof in the manufacture of a medicament for the treatment of a CBM complex pathway-associated disease or disorder.

242. The use of claim 241, wherein the CBM complex pathway-associated disease or disorder is selected from the group consisting of a CBM complex pathway cell surface receptor-associated cancer, a disease or disorder associated with a signal transducer between a cell surface receptor and a CBM complex, a component of a CBM complex-associated cancer, a MALT1 protease substrate-associated cancer, a disease or disorder associated with a component of the NF-?B pathway downstream of a CBM complex, a disease or disorder associated with a component of the JNK pathway downstream of a CBM complex, and a combination thereof.

243. The use of claim 241 or claim 242, wherein the CBM complex pathway-associated disease or disorder is a CBM complex pathway-associated autoimmune disorder.

244. The use of claim 241 or claim 242, wherein the CBM complex pathway-associated disease or disorder is a CBM complex pathway-associated inflammatory disorder.

245. The use of claim 241 or claim 242, wherein the CBM complex pathway-associated disease or disorder is a CBM complex pathway-associated cancer.

246. The use of any one of claims 241-245, wherein the CBM complex pathway-associated disease or disorder is a MALT1-associated disease or disorder.

247. The use of claim 246, wherein the MALT1-associated disease or disorder comprises a dysregulation of a MALT1 gene, a MALT1 protease protein, or expression or activity or level of any of the same.

248. The use of claim 247, wherein the dysregulation of a MALT1 gene, a MALT1 protease protein, or expression or activity or level of any of the same is an IAP2-MALT1 fusion, an IGH-MALT1 fusion, or a combination thereof.


SEQUENCE LISTING (36 sequences)

SEQ ID NO: 1 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 1271 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKNDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSNLYCTLEVDSFGYFVNKAKTRVYRDTAEPNWNEEFEIELEGSQTLRILCYEKCYNKTKIPKEDGESTDRLMGKGQVQLDPQALQDRDWQRTVIAMNGIEVKLSVKFNSREFSLKRMPSRKQTGVFGVKIAVVTKRERSKVPYIVRQCVEEIERRGMEEVGIYRVSGVATDIQALKAAFDVNNKDVSVMMSEMDVNAIAGTLKLYFRELPEPLFTDEFYPNFAEGIALSDPVAKESCMLNLLLSLPEANLLTFLFLLDHLKRVAEKEAVNKMSLHNLATVFGPTLLRPSEKESKLPANPSQPITMTDSWSLEVMSQVQVLLYFLQLEAIPAPDSKRQSILFSTEV

SEQ ID NO: 3 - protein, 1210 aa, Homo sapiens [sequence omitted]

SEQ ID NO: 4 - protein, 1255 aa, Homo sapiens [sequence omitted]

SEQ ID NO: 5 - protein, 671 aa, Homo sapiens
MADPAAGPPPSEGEESTVRFARKGALRQKNVHEVKNHKFTARFFKQPTFCSHCTDFIWGFGKQGFQCQVCCFVVHKRCHEFVTFSCPGADKGPASDDPRSKHKFKIHTYSSPTFCDHCGSLLYGLIHQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWFKLLSQEEGEYFNVPVPPEGSEANEELRQKFERAKISQGTKVPEEKTTNTVSKFDNNGNRDRMKLTDFNFLMVLGKGSFGKVMLSERKGTDELYAVKILKKDVVIQDDDVECTMVEKRVLALPGKPPFLTQLHSCFQTMDRLYFVMEYVNGGDLMYHIQQVGRFKEPHAVFYAAEIAIGLFFLQSKGIIYRDLKLDNVMLDSEGHIKIADFGMCKENIWDGVTTKTFCGTPDYIAPEIIAYQPYGKSVDWWAFGVLLYEMLAGQAPFEGEDEDELFQSIMEHNVAYPKSMSKEAVAICKGLMTKHPGKRLGCGPEGERDIKEHAFFRYIDWEKLERKEIQPPYKPKARDKRDTSNFDKEFTRQPVELTPTDKLFIMNLDQNEFAGFSYTNPEFVINV

SEQ ID NO: 6 - protein, 706 aa, Homo sapiens
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMNEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPPCLPTPGKREPQGISWESPLDEVDKMCHLPEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLIS

SEQ ID NO: 7 - protein, 824 aa, Homo sapiens
MSLLGDPLQALPPSAAPTGPLLAPPAGATLNRLREPLLRRLSELLDQAPEGRGWRRLAELAGSRGRLRLSCLDLEQCSLKVLEPEGSPSLCLLKLMGEKGCTVTELSDFLQAMEHTEVLQLLSPPGIKITVNPESKAVLAGQFVKLCCRATGHPFVQYQWFKMNKEIPNGNTSELIFNAVHVKDAGFYVCRVNNNFTFEFSQWSQLDVCDIPESFQRSVDGVSESKLQICVEPTSQKLMPGSTLVLQCVAVGSPIPHYQWFKNELPLTHETKKLYMVPYVDLEHQGTYWCHVYNDRDSQDSKKVEIIIGRTDEAVECTEDELNNLGHPDNKEQTTDQPLAKDKVALLIGNMNYREHPKLKAPLVDVYELTNLLRQLDFKVVSLLDLTEYEMRNAVDEFLLLLDKGVYGLLYYAGHGYENFGNSFMVPVDAPNPYRSENCLCVQNILKLMQEKETGLNVFLLDMCRKRNDYDDTIPILDALKVTANIVFGYATCQGAEAFEIQHSGLANGIFMKFLKDRLLEDKKITVLLDEVAEDMGKCHLTKGKQALEIRSSLSEKRALTDPIQGTEYSAESLVRNLQWAKAHELPESMCLKFDCGVQIQLGFAAEFSNVMIIYTSIVYKPPEIIMCDAYVTDFPLDLDIDPKDANKGTPEETGSYLVSKDLPKHCLYTRLSSLQKLKEHLVFTVCLSYQYSGLEDTVEDKQEVNVGKPLIAKLDMHRGLGRKTCFQTCLMSNGPYQSSAATSGGAGHYHSLQDPFHGVYHSHPGNPSNVTPADSCHCSRTPDAFISSFAHHASCHFSRSNVPVETTDEIPFSFSDRLRISEK

SEQ ID NO: 8 - protein, 1154 aa, Homo sapiens [sequence omitted]

SEQ ID NO: 9 - protein, 1004 aa, Homo sapiens
MGELCRRDSALTALDEETLWEMMESHRHRIVRCICPSRLTPYLRQAKVLCQLDEEEVLHSPRLTNSAMRAGHLLDLLKTRGKNGAIAFLESLKFHNPDVYTLVTGLQPDVDFSNFSGLMETSKLTECLAGAIGSLQEELNQEKGQKEVLLRRCQQLQEHLGLAETRAEGLHQLEADHSRMKREVSAHFHEVLRLKDEMLSLSLHYSNALQEKELAASRCRSLQEELYLLKQELQRANMVSSCELELQEQSLRTASDQESGDEELNRLKEENEKLRSLTFSLAEKDILEQSLDEARGSRQELVERIHSLRERAVAAERQREQYWEEKEQTLLQFQKSKMACQLYREKVNALQAQVCELQKERDQAYSARDSAQREISQSLVEKDSLRRQVFELTDQVCELRTQLRQLQAEPPGVLKQEARTREPCPREKQRLVRMHAICPRDDSDCSLVSSTESQLLSDLSATSSRELVDSFRSSSPAPPSQQSLYKRVAEDFGEEPWSFSSCLEIPEGDPGALPGAKAGDPHLDYELLDTADLPQLESSLQPVSPGRLDVSESGVLMRRRPARRILSQVTMLAFQGDALLEQISVIGGNLTGIFIHRVTPGSAADQMALRPGTQIVMVDYEASEPLFKAVLEDTTLEEAVGLLRRVDGFCCLSVKVNTDGYKRLLQDLEAKVATSGDSFYIRVNLAMEGRAKGELQVHCNEVLHVTDTMFQGCGCWHAHRVNSYTMKDTAAHGTIPNYSRAQQQLIALIQDMTQQCTVTRKPSSGGPQKLVRIVSMDKAKASPLRLSFDRGQLDPSRMEGSSTCFWAESCLTLVPYTLVRPHRPARPRPVLLVPRAVGKILSEKLCLLQGFKKCLAEYLSQEEYEAWSQRGDIIQEGEVSGGRCWVTRHAVESLMEKNTHALLDVQLDSVCTLHRMDIFPIVIHVSVNEKMAKKLKKGLQRLGTSEEQLLEAARQEEGDLDRAPCLYSSLAPDGWSDLDGLLSCVRQAIADEQKKVVWTEQSPR

SEQ ID NO: 10 - protein, 1032 aa, Homo sapiens
MPGRAEAGEAEEEAGAGSGSEAEEDALWERIEGVRHRLARALNPAKLTPYLRQCRVIDEQDEEEVLSTYRFPCRVNRTGRLMDILRCRGKRGYEAFLEALEFYYPEHFTLLTGQEPAQRCSMILDEEGPEGLTQFLMTEVRRLREARKSQLQREQQLQARGRVLEEERAGLEQRLRDQQQAQERCQRLREDWEAGSLELLRLKDENYMIAMRLAQLSEEKNSAVLRSRDLQLAVDQLKLKVSRLEEECALLRRARGPPPGAEEKEKEKEKEKEPDNVDLVSELRAENQRLTASLRELQEGLQQEASRPGAPGSERILLDILEHDWREAQDSRQELCQKLHAVQGELQWAEELRDQYLQEMEDLRLKHRTLQKDCDLYKHRMATVLAQLEEIEKERDQAIQSRDRIQLQYSQSLIEKDQYRKQVRGLEAERDELLTTLTSLEGTKALLEVQLQRAQGGTCLKACASSHSLCSNLSSTWSLSEFPSPLGGPEATGEAAVMGGPEPHNSEEATDSEKEINRLSILPFPPSAGSILRRQREEDPAPPKRSFSSMSDITGSVTLKPWSPGLSSSSSSDSVWPLGKPEGLLARGCGLDFLNRSLAIRVSGRSPPGGPEPQDKGPDGLSFYGDRWSGAVVRRVLSGPGSARMEPREQRVEAAGLEGACLEAEAQQRTLLWNQGSTLPSLMDSKACQSFHEALEAWAKGPGAEPFYIRANLTLPERADPHALCVKAQEILRLVDSAYKRRQEWFCTRVDPLTLRDLDRGTVPNYQRAQQLLEVQEKCLPSSRHRGPRSNLKKRALDQLRLVRPKPVGAPAGDSPDQLLLEPCAEPERSLRPYSLVRPLLVSALRPVVLLPECLAPRLIRNLLDLPSSRLDFQVCPAESLSGEELCPSSAPGAPKAQPATPGLGSRIRAIQESVGKKHCLLELGARGVRELVQNEIYPIVIHVEVTEKNVREVRGLLGRPGWRDSELLRQCRGSEQVLWGLPCSWVQVPAHEWGHAEELAKVVRGRILQEQARLVWVECGSSRGCPSSSEA

SEQ ID NO: 11 - protein, 536 aa, Homo sapiens
MSDYENDDECWSVLEGFRVTLTSVIDPSRITPYLRQCKVLNPDDEEQVLSDPNLVIRKRKVGVLLDILQRTGHKGYVAFLESLELYYPQLYKKVTGKEPARVFSMIIDASGESGLTQLLMTEVMKLQKKVQDLTALLSSKDDFIKELRVKDSLLRKHQERVQRLKEECEAGSRELKRCKEENYDLAMRLAHQSEEKGAALMRNRDLQLEIDQLKHSLMKAEDDCKVERKHTLKLRHAMEQRPSQELLWELQQEKALLQARVQELEASVQEGKLDRSSPYIQVLEEDWRQALRDHQEQANTIFSLRKDLRQGEARRLRCMEEKEMFELQCLALRKDSKMYKDRIEAILLQMEEVAIERDQAIATREELHAQHARGLQEKDALRKQVRELGEKADELQLQVFQCEAQLLAVEGRLRRQQLETLVLSSDLEDGSPRRSQELSLPQDLEDTQLSDKGCLAGGGSPKQPFAALHQEQVLRNPHDAGLSSGEPPEKERRRLKESFENYRRKRALRKMQKGWRQGEEDRENTTGSDNTDTEGS

SEQ ID NO: 12 - protein, 233 aa, Homo sapiens
MEPTAPSLTEEDLTEVKKDALENLRVYLCEKIIAERHFDHLRAKKILSREDTEEISCRTSSRKRAGKLLDYLQENPKGLDTLVESIRREKTQNFLIQKITDEVLKLRNIKLEHLKGLKCSSCEPFPDGATNNLSRSNSDESNFSEKLRASTVMYHPEGESSTTPFFSTNSSLNLPVLEVGRTENTIFSSTTLPRPGDPGAPPLPPDLQLEEEGTCANSSEMFLPLRSRTVSRQ

SEQ ID NO: 13 - protein, 790 aa, Homo sapiens
MAEQVLPQALYLSNMRKAVKIRERTPEDIFKPTNGIIHHFKTMHRYTLEMFRTCQFCPQFREIIHKALIDRNIQATLESQKKLNWCREVRKLVALKTNGDGNCLMHATSQYMWGVQDTDLVLRKALFSTLKETDTRNFKFRWQLESLKSQEFVETGLCYDTRNWNDEWDNLIKMASTDTPMARSGLQYNSLEEIHIFVLCNILRRPIIVISDKMLRSLESGSNFAPLKVGGIYLPLHWPAQECYRYPIVLGYDSHHFVPLVTLKDSGPEIRAVPLVNRDRGRFEDLKVHFLTDPENEMKEKLLKEYLMVIEIPVQGWDHGTTHLINAAKLDEANLPKEINLVDDYFELVQHEYKKWQENSEQGRREGHAQNPMEPSVPQLSLMDVKCETPNCPFFMSVNTQPLCHECSERRQKNQNKLPKLNSKPGPEGLPGMALGASRGEAYEPLAWNPEESTGGPHSAPPTAPSPFLFSETTAMKCRSPGCPFTLNVQHNGFCERCHNARQLHASHAPDHTRHLDPGKCQACLQDVTRTFNGICSTCFKRTTAEASSSLSTSLPPSCHQRSKSDPSRLVRSPSPHSCHRAGNDAPAGCLSQAARTPGDRTGTSKCRKAGCVYFGTPENKGFCTLCFIEYRENKHFAAASGKVSPTASRFQNTIPCLGRECGTLGSTMFEGYCQKCFIEAQNQRFHEAKRTEEQLRSSQRRDVPRTTQSTSRPKCARASCKNILACRSEELCMECQHPNQRMGPGAHRGEPAPEDPPKQRCRAPACDHFGNAKCNGYCNECFQFKQMYG

SEQ ID NO: 14 - protein, 956 aa, Homo sapiens
MSSGLWSQEKVTSPYWEERIFYLLLQECSVTDKQTQKLLKVPKGSIGQYIQDRSVGHSRIPSAKGKKNQIGLKILEQPHAVLFVDEKDVVEINEKFTELLLAITNCEERFSLFKNRNRLSKGLQIDVGCPVKVQLRSGEEKFPGVVRFRGPLLAERTVSGIFFGVELLEEGRGQGFTDGVYQGKQLFQCDEDCGVFVALDKLELIEDDDTALESDYAGPGDTMQVELPPLEINSRVSLKVGETIESGTVIFCDVLPGKESLGYFVGVDMDNPIGNWDGRFDGVQLCSFACVESTILLHINDIIPALSESVTQERRPPKLAFMSRGVGDKGSSSHNKPKATGSTSDPGNRNRSELFYTLNGSSVDSQPQSKSKNTWYIDEVAEDPAKSLTEISTDFDRSSPPLQPPPVNSLTTENRFHSLPFSLTKMPNTNGSIGHSPLSLSAQSVMEELNTAPVQESPPLAMPPGNSHGLEVGSLAEVKENPPFYGVIRWIGQPPGLNEVLAGLELEDECAGCTDGTFRGTRYFTCALKKALFVKLKSCRPDSRFASLQPVSNQIERCNSLAFGGYLSEVVEENTPPKMEKEGLEIMIGKKKGIQGHYNSCYLDSTLFCLFAFSSVLDTVLLRPKEKNDVEYYSETQELLRTEIVNPLRIYGYVCATKIMKLRKILEKVEAASGFTSEEKDPEEFLNILFHHILRVEPLLKIRSAGQKVQDCYFYQIFMEKNEKVGVPTIQQLLEWSFINSNLKFAEAPSCLIIQMPRFGKDFKLFKKIFPSLELNITDLLEDTPRQCRICGGLAMYECRECYDDPDISAGKIKQFCKTCNTQVHLHPKRLNHKYNPVSLPKDLPDWDWRHGCIPCQNMELFAVLCIETSHYVAFVKYGKDDSAWLFFDSMADRDGGQNGFNIPQVTPCPEVGEYLKMSLEDLHSLDSRRIQGCARRLLCDAYMCMYQSPTMSLYK

SEQ ID NO: 15 - protein, 579 aa, Homo sapiens
MLRSGPASGPSVPTGRAMPSRRVARPPAAPELGALGSPDLSSLSLAVSRSTDELEIIDEYIKENGFGLDGGQPGPGEGLPRLVSRGAASLSTVTLGPVAPPATPPPWGCPLGRLVSPAPGPGPQPHLVITEQPKQRGMRFRYECEGRSAGSILGESSTEASKTLPAIELRDCGGLREVEVTACLVWKDWPHRVHPHSLVGKDCTDGICRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPNHGSGPFLPPSALLPDPDFFSGTVSLPGLEPPGGPDLLDDGFAYDPTAPTLFTMLDLLPPAPPHASAVVCSGGAGAVVGETPGPEPLTLDSYQAPGPGDGGTASLVGSNMFPNHYREAAFGGGLLSPGPEAT

SEQ ID NO: 16 - protein, 599 aa, Homo sapiens
MSGPCGEKPVLEASPTMSLWEFEDSHSRQGTPRPGQELAAEEASALELQMKVDFFRKLGYSSTEIHSVLQKLGVQADTNTVLGELVKHGTATERERQTSPDPCPQLPLVPRGGGTPKAPNLEPPLPEEEKEGSDLRPVVIDGSNVAMSHGNKEVFSCRGILLAVNWFLERGHTDITVFVPSWRKEQPRPDVPITDQHILRELEKKKILVFTPSRRVGGKRVVCYDDRFIVKLAYESDGIVVSNDTYRDLQGERQEWKRFIEERLLMYSFVNDKFMPPDDPLGRHGPSLDNFLRKKPLTLEHRKQPCPYGRKCTYGIKCRFFHPERPSCPQRSVADELRANALLSPPRAPSKDKNGRRPSPSSQSSSLLTESEQCSLDGKKLGAQASPGSRQEGLTQTYAPSGRSLAPSGGSGSSFGPTDWLPQTLDSLPYVSQDCLDSGIGSLESQMSELWGVRGGGPGEPGPPRAPYTGYSPYGSELPATAAFSAFGRAMGAGHFSVPADYPPAPPAFPPREYWSEPYPLPPPTSVLQEPPVQSPGAGRSPWGRAGSLAKEQASVYTKLCGVFPPHLVEAVMGRFPQLLDPQQLAAEILSYKSQHPSE

SEQ ID NO: 17 - protein, 1133 aa, Homo sapiens
MPVQAPQWTDFLSCPICTQTFDETIRKPISLGCGHTVCKMCLNKLHRKACPFDQTTINTDIELLPVNSALLQLVGAQVPEQQPITLCSGVEDTKHYEEAKKCVEELALYLKPLSSARGVGLNSTTQSVLSRPMQRKLVTLVHCQLVEEEGRIRAMRAARSLGERTVTELILQHQNPQQLSSNLWAAVRARGCQFLGPAMQEEALKLVLLALEDGSALSRKVLVLFVVQRLEPRFPQASKTSIGHVVQLLYRASCFKVTKRDEDSSLMQLKEEFRTYEALRREHDSQIVQIAMEAGLRIAPDQWSSLLYGDQSHKSHMQSIIDKLQTPASFAQSVQELTIALQRTGDPANLNRLRPHLELLANIDPSPDAPPPTWEQLENGLVAVRTVVHGLVDYIQNHSKKGADQQQPPQHSKYKTYMCRDMKQRGGCPRGASCTFAHSQEELEKFRKMNKRLVPRRPLSASLGQLNEVGLPSAAILPDEGAVDLPSRKPPALPNGIVSTGNTVTQLIPRGTDPSYDSSLKPGKIDHLSSSAPGSPPDLLESVPKSISALPVNPHSIPPRGPADLPPMPVTKPLQMVPRGSQLYPAQQTDVYYQDPRGAAPPFEPAPYQQGMYYTPPPQCVSRFVRPPPSAPEPAPPYLDHYPPYLQERVVNSQYGTQPQQYPPIYPSHYDGRRVYPAPSYTREEIFRESPIPIEIPPAAVPSYVPESRERYQQIESYYPVAPHPTQIRPSYLREPPYSRLPPPPQPHPSLDELHRRRKEIMAQLEERKVISPPPFAPSPTLPPTFHPEEFLDEDLKVAGKYKGNDYSQYSPWSCDTIGSYIGTKDAKPKDVVAAGSVEMMNVESKGMRDQRLDLQRRAAETSDDDLIPFGDRPTVSRFGAISRTSKTIYQGAGPMQAMAPQGAPTKSINISDYSPYGTHGGWGASPYSPHQNIPSQGHFSERERISMSEVASHGKPLPSAEREQLRLELQQLNHQISQQTQLRGLEAVSNRLVLQREANTLAGQSQPPPPPPPKWPGMISSEQLSLELHQVEREIGKRTRELSMENQCSLDMKSKLNTSKQAENGQPEPQNKVPAEDLTLTFSDVPNGSALTQENISLLSNKTSSLNLSEDPEGGGDNNDSQRSGVTPSSAP

SEQ ID NO: 18 - protein, 510 aa, Homo sapiens
MDEKTKKAEEMALSLTRAVAGGDEQVAMKCAIWLAEQRVPLSVQLKPEVSPTQDIRLWVSVEDAQMHTVTIWLTVRPDMTVASLKDMVFLDYGFPPVLQQWVIGQRLARDQETLHSHGVRQNGDSAYLYLLSARNTSLNPQELQRERQLRMLEDLGFKDLTLQPRGPLEPGPPKPGVPQEPGRGQPDAVPEPPPVGWQCPGCTFINKPTRPGCEMCCRARPEAYQVPASYQPDEEERARLAGEEEALRQYQQRKQQQQEGNYLQHVQLDQRSLVLNTEPAECPVCYSVLAPGEAVVLRECLHTFCRECLQGTIRNSQEAEVSCPFIDNTYSCSGKLLEREIKALLTPEDYQRFLDLGISIAENRSAFSYHCKTPDCKGWCFFEDDVNEFTCPVCFHVNCLLCKAIHEQMNCKEYQEDLALRAQNDVAARQTTEMLKVMLQQGEAMRCPQCQIVVQKKDGCDWIRCTVCHTEICWVTKGPRWGPGGPGDTSGGCRCRVNGIPCHPSCQNCH

SEQ ID NO: 19 - protein, 947 aa, Homo sapiens
MAVMEMACPGAPGSAVGQQKELPKAKEKTPPLGKKQSSVYKLEAVEKSPVFCGKWEILNDVITKGTAKEGSEAGPAAISIIAQAECENSQEFSPTFSERIFIAGSKQYSQSESLDQIPNNVAHATEGKMARVCWKGKRRSKARKKRKKKSSKSLAHAGVALAKPLPRTPEQESCTIPVQEDESPLGAPYVRNTPQFTKPLKEPGLGQLCFKQLGEGLRPALPRSELHKLISPLQCLNHVWKLHHPQDGGPLPLPTHPFPYSRLPHPFPFHPLQPWKPHPLESFLGKLACVDSQKPLPDPHLSKLACVDSPKPLPGPHLEPSCLSRGAHEKFSVEEYLVHALQGSVSSGQAHSLTSLAKTWAARGSRSREPSPKTEDNEGVLLTEKLKPVDYEYREEVHWATHQLRLGRGSFGEVHRMEDKQTGFQCAVKKVRLEVFRAEELMACAGLTSPRIVPLYGAVREGPWVNIFMELLEGGSLGQLVKEQGCLPEDRALYYLGQALEGLEYLHSRRILHGDVKADNVLLSSDGSHAALCDFGHAVCLQPDGLGKSLLTGDYIPGTETHMAPEVVLGRSCDAKVDVWSSCCMMLHMLNGCHPWTQFFRGPLCLKIASEPPPVREIPPSCAPLTAQAIQEGLRKEPIHRVSAAELGGKVNRALQQVGGLKSPWRGEYKEPRHPPPNQANYHQTLHAQPRELSPRAPGPRPAEETTGRAPKLQPPLPPEPPEPNKSPPLTLSKEESGMWEPLPLSSLEPAPARNPSSPERKATVPEQELQQLEIELFLNSLSQPFSLEEQEQILSCLSIDSLSLSDDSEKNPSKASQSSRDTLSSGVHSWSSQAEARSSSWNMVLARGRPTDTPSYFNGVKVQIQSLNGEHLHIREFHRVKVGDIATGISSQIPAAAFSLVTKDGQPVRYDMEVPDSGIDLQCTLAPDGSFAWSWRVKHGQLENRP

SEQ ID NO: 20 - protein, 599 aa, Homo sapiens
MENCLGESRHEVEKSEISENTDASGKIEKYNVPLNRLKMMFEKGEPTQTKILRAQSRSASGRKISENSYSLDDLEIGPGQLSSSTFDSEKNESRRNLELPRLSETSIKDRMAKYQAAVSKQSSSTNYTNELKASGGEIKIHKMEQKENVPPGPEVCITHQEGEKISANENSLAVRSTPAEDDSRDSQVKSEVQQPVHPKPLSPDSRASSLSESSPPKAMKKFQAPARETCVECQKTVYPMERLLANQQVFHISCFRCSYCNNKLSLGTYASLHGRIYCKPHFNQLFKSKGNYDEGFGHRPHKDLWASKNENEEILERPAQLANARETPHSPGVEDAPIAKVGVLAASMEAKASSQQEKEDKPAETKKLRIAWPPPTELGSSGSALEEGIKMSKPKWPPEDEISKPEVPEDVDLDLKKLRRSSSLKERSRPFTVAASFQSTSVKSPKTVSPPIRKGWSMSEQSEESVGGRVAERKQVENAKASKKNGNVGKTTWQNKESKGETGKRSKEGHSLEMENENLVENGADSDEDDNSFLKQQSPQEPKSLNWSSFVDNTFAEEFTTQNQKSQDVELWEGEVVKELSVEEQIKRNRYYDEDEDEE

SEQ ID NO: 21 - protein, 606 aa, Homo sapiens
MSTASAASSSSSSSAGEMIEAPSQVLNFEEIDYKEIEVEEVVGRGAFGVVCKAKWRAKDVAIKQIESESERKAFIVELRQLSRVNHPNIVKLYGACLNPVCLVMEYAEGGSLYNVLHGAEPLPYYTAAHAMSWCLQCSQGVAYLHSMQPKALIHRDLKPPNLLLVAGGTVLKICDFGTACDIQTHMTNNKGSAAWMAPEVFEGSNYSEKCDVFSWGIILWEVITRRKPFDEIGGPAFRIMWAVHNGTRPPLIKNLPKPIESLMTRCWSKDPSQRPSMEEIVKIMTHLMRYFPGADEPLQYPCQYSDEGQSNSATSTGSFMDIASTNTSNKSDTNMEQVPATNDTIKRLESKLLKNQAKQQSESGRLSLGASRGSSVESLPPTSEGKRMSADMSEIEARIAATTAYSKPKRGHRKTASFGNILDVPEIVISGNGQPRRRSIQDLTVTGTEPGQVSSRSSSPSVRMITTSGPTSEKPTRSHPWTPDDSTDTNGSDNSIPMAYLTLDHQLQPLAPCPNSKESMAVFEQHCKMAQEYMKVQTEIALLLQRKQELVAELDQDEKDQQNTSRLVQEHKKLLDENKSLSTYYQQCKKQLEVIRSQQQKRQGTS

SEQ ID NO: 22 - protein, 522 aa, Homo sapiens
MSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAQAVHSLSVIPDSGYISEVRNFQETIHQLEGRLVRQDHQIRELTAKMETQSMYVSELKRTIRTLEDKVAEIEAQQCNGIYIWKIGNFGMHLKCQEEEKPVVIHSPGFYTGKPGYKLCMRLHLQLPTAQRCANYISLFVHTMQGEYDSHLPWPFQGTIRLTILDQSEAPVRQNHEEIMDAKPELLAFQRPTIPRNPKGFGYVTFMHLEALRQRTFIKDDTLLVRCEVSTRFDMGSLRREGFQPRSTDAGV

SEQ ID NO: 23 - protein, 504 aa, Homo sapiens
MAAQRRSLLQSEQQPSWTDDLPLCHLSGVGSASNRSYSADGKGTESHPPEDSWLKFRSENNCFLYGVFNGYDGNRVTNFVAQRLSAELLLGQLNAEHAEADVRRVLLQAFDVVERSFLESIDDALAEKASLQSQLPEGVPQHQLPPQYQKILERLKTLEREISGGAMAVVAVLLNNKLYVANVGTNRALLCKSTVDGLQVTQLNVDHTTENEDELFRLSQLGLDAGKIKQVGIICGQESTRRIGDYKVKYGYTDIDLLSAAKSKPIIAEPEIHGAQPLDGVTGFLVLMSEGLYKALEAAHGPGQANQEIAAMIDTEFAKQTSLDAVAQAVVDRVKRIHSDTFASGGERARFCPRHEDMTLLVRNFGYPLGEMSQPTPSPAPAAGGRVYPVSVPYSSAQSTSKTSVTLSLVMPSQGQMVNGAHSASTLDEATPTLTNQSPTLTLQSTNTHTQSSSSSSDGGLFRSRPAHSLPPGEDGRVEPYVDFAEFYRLWSVDHGEQSVVTAP

SEQ ID NO: 24 - protein, 693 aa, Homo sapiens
MAQGSHQIDFQVLHDLRQKFPEVPEVVVSRCMLQNNNNLDACCAVLSQESTRYLYGEGDLNFSDDSGISGLRNHMTSLNLDLQSQNIYHHGREGSRMNGSRTLTHSISDGQLQGGQSNSELFQQEPQTAPAQVPQGFNVFGMSSSSGASNSAPHLGFHLGSKGTSSLSQQTPRFNPIMVTLAPNIQTGRNTPTSLHIHGVPPPVLNSPQGNSIYIRPYITTPGGTTRQTQQHSGWVSQFNPMNPQQVYQPSQPGPWTTCPASNPLSHTSSQQPNQQGHQTSHVYMPISSPTTSQPPTIHSSGSSQSSAHSQYNIQNISTGPRKNQIEIKLEPPQRNNSSKLRSSGPRTSSTSSSVNSQTLNRNQPTVYIAASPPNTDELMSRSQPKVYISANAATGDEQVMRNQPTLFISTNSGASAASRNMSGQVSMGPAFIHHHPPKSRAIGNNSATSPRVVVTQPNTKYTFKITVSPNKPPAVSPGVVSPTFELTNLLNHPDHYVETENIQHLTDPTLAHVDRISETRKLSMGSDDAAYTQALLVHQKARMERLQRELEIQKKKLDKLKSEVNEMENNLTRRRLKRSNSISQIPSLEEMQQLRSCNRQLQIDIDCLTKEIDLFQARGPHFNPSAIHNFYDNIGFVGPVPPKPKDQRSIIKTPKTQDTEDDEGAQWNCTACTFLNHPALIRCEQCEMPRHF

SEQ ID NO: 25 - protein, 712 aa, Homo sapiens
MAQSSPQLDIQVLHDLRQRFPEIPEGVVSQCMLQNNNNLEACCRALSQESSKYLYMEYHSPDDNRMNRNRLLHINLGIHSPSSYHPGDGAQLNGGRTLVHSSSDGHIDPQHAAGKQLICLVQEPHSAPAVVAATPNYNPFFMNEQNRSAATPPSQPPQQPSSMQTGMNPSAMQGPSPPPPPPSYMHIPRYSTNPITVTVSQNLPSGQTVPRALQILPQIPSNLYGSPGSIYIRQTSQSSSGRQTPQSTPWQSSPQGPVPHYSQRPLPVYPHQQNYQPSQYSPKQQQIPQSAYHSPPPSQCPSPFSSPQHQVQPSQLGHIFMPPSPSTTPPHPYQQGPPSYQKQGSHSVAYLPYTASSLSKGSMKKIEITVEPSQRPGTAINRSPSPISNQPSPRNQHSLYTATTPPSSSPSRGISSQPKPPFSVNPVYITYTQPTGPSCTPSPSPRVIPNPTTVFKITVGRATTENLLNLVDQEERSAAPEPIQPISVIPGSGGEKGSHKYQRSSSSGSDDYAYTQALLLHQRARMERLAKQLKLEKEELERLKSEVNGMEHDLMQRRLRRVSCTTAIPTPEEMTRLRSMNRQLQINVDCTLKEVDLLQSRGNFDPKAMNNFYDNIEPGPVVPPKPSKKDSSDPCTIERKARRISVTSKVQADIHDTQAAAADEHRTGSTQSPRTQPRDEDYEGAPWNCDSCTFLNHPALNRCEQCEMPRYT

SEQ ID NO: 26 - protein, 419 aa, Homo sapiens
MAASSLEQKLSRLEAKLKQENREARRRIDLNLDISPQRPRPTLQLPLANDGGSRSPSSESSPQHPTPPARPRHMLGLPSTLFTPRSMESIEIDQKLQEIMKQTGYLTIGGQRYQAEINDLENLGEMGSGTCGQVWKMRFRKTGHVIAVKQMRRSGNKEENKRILMDLDVVLKSHDCPYIVQCFGTFITNTDVFIAMELMGTCAEKLKKRMQGPIPERILGKMTVAIVKALYYLKEKHGVIHRDVKPSNILLDERGQIKLCDFGISGRLVDSKAKTRSAGCAAYMAPERIDPPDPTKPDYDIRADVWSLGISLVELATGQFPYKNCKTDFEVLTKVLQEEPPLLPGHMGFSGDFQSFVKDCLTKDHRKRPKYNKLLEHSFIKRYETLEVDVASWFKDVMAKTESPRTSGVLSQPHLPFFR

SEQ ID NO: 27 - protein, 745 aa, Homo sapiens
MERPPGLRPGAGGPWEMRERLGTGGFGNVCLYQHRELDLKIAIKSCRLELSTKNRERWCHEIQIMKKLNHANVVKACDVPEELNILIHDVPLLAMEYCSGGDLRKLLNKPENCCGLKESQILSLLSDIGSGIRYLHENKIIHRDLKPENIVLQDVGGKIIHKIIDLGYAKDVDQGSLCTSFVGTLQYLAPELFENKPYTATVDYWSFGTMVFECIAGYRPFLHHLQPFTWHEKIKKKDPKCIFACEEMSGEVRFSSHLPQPNSLCSLVVEPMENWLQLMLNWDPQQRGGPVDLTLKQPRCFVLMDHILNLKIVHILNMTSAKIISFLLPPDESLHSLQSRIERETGINTGSQELLSETGISLDPRKPASQCVLDGVRGCDSYMVYLFDKSKTVYEGPFASRSLSDCVNYIVQDSKIQLPIIQLRKVWAEAVHYVSGLKEDYSRLFQGQRAAMLSLLRYNANLTKMKNTLISASQQLKAKLEFFHKSIQLDLERYSEQMTYGISSEKMLKAWKEMEEKAIHYAEVGVIGYLEDQIMSLHAEIMELQKSPYGRRQGDLMESLEQRAIDLYKQLKHRPSDHSYSDSTEMVKIIVHTVQSQDRVLKELFGHLSKLLGCKQKIIDLLPKVEVALSNIKEADNTVMFMQGKRQKEIWHLLKIACTQSSARSLVGSSLEGAVTPQTSAWLPPTSAEHDHSLSCVVTPQDGETSAQMIEENLNCLGHLSTIIHEANEEQGNSMMNLDWSWLTE

SEQ ID NO: 28 - protein, 756 aa, Homo sapiens
MSWSPSLTTQTCGAWEMKERLGTGGFGNVIRWHNQETGEQIAIKQCRQELSPRNRERWCLEIQIMRRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQWHSKVRQKSEVDIVVSEDLNGTVKFSSSLPYPNNLNSVLAERLEKWLQLMLMWHPRQRGTDPTYGPNGCFKALDDILNLKLVHILNMVTGTIHTYPVTEDESLQSLKARIQQDTGIPEEDQELLQEAGLALIPDKPATQCISDGKLNEGHTLDMDLVFLFDNSKITYETQISPRPQPESVSCILQEPKRNLAFFQLRKVWGQVWHSIQTLKEDCNRLQQGQRAAMMNLLRNNSCLSKMKNSMASMSQQLKAKLDFFKTSIQIDLEKYSEQTEFGITSDKLLLAWREMEQAVELCGRENEVKLLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDEKTVVRLQEKRQKELWNLLKIACSKVRGPVSGSPDSMNASRLSQPGQLMSQPSTASNSLPEPAKKSEELVAEAHNLCTLLENAIQDTVREQDQSFTALDWSWLQTEEEEHSCLEQAS

SEQ ID NO: 29 - protein, 419 aa, Homo sapiens
MNRHLWKSQLCEMVQPSGGPAADQDVLGEESPLGKPAMLHLPSEQGAPETLQRCLEENQELRDAIRQSNQILRERCEELLHFQASQREEKEFLMCKFQEARKLVERLGLEKLDLKRQKEQALREVEHLKRCQQQMAEDKASVKAQVTSLLGELQESQSRLEAATKECQALEGRARAASEQARQLESEREALQQQHSVQVDQLRMQGQSVEAALRMERQAASEEKRKLAQLQVAYHQLFQEYDNHIKSSVVGSERKRGMQLEDLKQQLQQAEEALVAKQEVIDKLKEEAEQHKIVMETVPVLKAQADIYKADFQAERQAREKLAEKKELLQEQLEQLQREYSKLKASCQESARIEDMRKRHVEVSQAPLPPAPAYLSSPLALPSQRRSPPEEPPDFCCPKCQYQAPDMDTLQIHVMECIE

SEQ ID NO: 30 - protein, 317 aa, Homo sapiens
MFQAAERPQEWAMEGPRDGLKKERLLDDRHDSGLDSMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 31 - protein, 968 aa, Homo sapiens
MAEDDPYLGRPEQMFHLDPSLTHTIFNPEVFQPQMALPTDGPYLQILEQPKQRGFRFRYVCEGPSHGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGYNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKLMPNFSDSFGGGSGAGAGGGGMFGSGGGGGGTGSTGPGYSFPHYGFPTYGGITFHPGTTKSNAGMKHGTMDTESKKDPEGCDKSDDKNTVNLFGKVIETTEQDQEPSEATVGNGEVTLTYATGTKEESAGVQDNLFLEKAMQLAKRHANALFDYAVTGDVKMLLAVQRHLTAVQDENGDSVLHLAIIHLHSQLVRDLLEVTSGLISDDIINMRNDLYQTPLHLAVITKQEDVVEDLLRAGADLSLLDRLGNSVLHLAAKEGHDKVLSILLKHKKAALLLDHPNGDGLNAIHLAMMSNSLPCLLLLVAAGADVNAQEQKSGRTALHLAVEHDNISLAGCLLLEGDAHVDSTTYDGTTPLHIAAGRGSTRLAALLKAAGADPLVENFEPLYDLDDSWENAGEDEGVVPGTTPLDMATSWQVFDILNGKPYEPEFTSDDLLAQGDMKQLAEDVKLQLYKLLEIPDPDKNWATLAQKLGLGILNNAFRLSPAPSKTLMDNYEVSGGTVRELVEALRQMGYTEAIEVIQAASSPVKTTSQAHSLPLSPASTRQQIDELRDSDSVCDSGVETSFRKLSFTESLTSGASLLTLNKMPHDYGQEGPLEGKI

SEQ ID NO: 32 - protein, 551 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 33 - protein, 619 aa, Homo sapiens
MASGAYNPYIEIIEQPRQRGMRFRYKCEGRSAGSIPGEHSTDNNRTYPSIQIMNYYGKGKVRITLVTKNDPYKPHPHDLVGKDCRDGYYEAEFGQERRPLFFQNLGIRCVKKKEVKEAIITRIKAGINPFNVPEKQLNDIEDCDLNVVRLCFQVFLPDEHGNLTTALPPVVSNPIYDNRAPNTAELRICRVNKNCGSVRGGDEIFLLCDKVQKDDIEVRFVLNDWEAKGIFSQADVHRQVAIVFKTPPYCKAITEPVTVKMQLRRPSDQEVSESMDFRYLPDEKDTYGNKAKKQKTTLLFQKLCQDHVETGFRHVDQDGLELLTSGDPPTLASQSAGITVNFPERPRPGLLGSIGEGRYFKKEPNLFSHDAVVREMPTGVSSQAESYYPSPGPISSGLSHHASMAPLPSSSWSSVAHPTPRSGNTNPLSSFSTRTLPSNSQGIPPFLRIPVGNDLNASNACIYNNADDIVGMEASSMPSADLYGISDPNMLSNCSVNMMTTSSDSMGETDNPRLLSMNLENPSCNSVLDPRDLRQLHQMSSSSMSAGANSNTTVFVSQSDAFEGSDFSCADNSMINESGPSNSTNPNSHGFVQDSQYSGIGSMQNEQLSDSFPYEFFQV

SEQ ID NO: 34 - protein, 427 aa, Homo sapiens
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDLWSVGCIMGEMVCHKILFPGRDYIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLGAAVINGSQHPSSSSSVNDVSSMSTDPTLASDTDSSLEAAAGPLGCCR

SEQ ID NO: 35 - protein, 424 aa, Homo sapiens
MSDSKCDSQFYSVQVADSTFTVLKRYQQLKPIGSGAQGIVCAAFDTVLGINVAVKKLSRPFQNQTHAKRAYRELVLLKCVNHKNIISLLNVFTPQKTLEEFQDVYLVMELMDANLCQVIHMELDHERMSYLLYQMLCGIKHLHSAGIIHRDLKPSNIVVKSDCTLKILDFGLARTACTNFMMTPYVVTRYYRAPEVILGMGYKENVDIWSVGCIMGELVKGCVIFQGTDHIDQWNKVIEQLGTPSAEFMKKLQPTVRNYVENRPKYPGIKFEELFPDWIFPSESERDKIKTSQARDLLSKMLVIDPDKRISVDEALRHPYITVWYDPAEAEAPPPQIYDAQLEEREHAIEEWKELIYKEVMDWEERSKNGVVKDQPSDAAVSSNATPSQSSSINDISSMSTEQTLASDTDSSLDASTGPLEGCR

SEQ ID NO: 36 - protein, 464 aa, Homo sapiens
MSLHFLYYCSEPTLDVKIAFCQGFDKQVDVSYIAKHYNMSKSKVDNQFYSVEVGDSTFTVLKRYQNLKPIGSGAQGIVCAAYDAVLDRNVAIKKLSRPFQNQTHAKRAYRELVLMKCVNHKNIISLLNVFTPQKTLEEFQDVYLVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDIWSVGCIMGEMVRHKILFPGRDYIDQWNKVIEQLGTPCPEFMKKLQPTVRNYVENRPKYAGLTFPKLFPDSLFPADSEHNKLKASQARDLLSKMLVIDPAKRISVDDALQHPYINVWYDPAEVEAPPPQIYDKQLDEREHTIEEWKELIYKEVMNSEEKTKNGVVKGQPSPSGAAVNSSESLPPSSSVNDISSMSTDQTLASDTDSSLEASAGPLGCCR